(19) United States
(12) Patent Application Publication
(10) Pub. No.: US 2025/0114345 A1
(43) Pub. Date: Apr. 10, 2025

(54) COMPOUNDS AND COMPOSITIONS AS CBP/P300 DEGRADERS AND USES THEREOF

(71) Applicant: Oncopia Therapeutics, Inc. d/b/a/ SK Life Science Labs, King Of Prussia, PA (US)

(72) Inventors: Xuqing ZHANG, King of Prussia, PA (US); Jeremy ROACH, King of Prussia, PA (US); Qiaolin DENG, King of Prussia, PA (US); E. Scott PRIESTLEY, King of Prussia, PA (US)

(21) Appl. No.: 18/821,490

(22) Filed: Aug. 30, 2024

Related U.S. Application Data
(60) Provisional application No. 63/579,540, filed on Aug. 30, 2023.

(51) Int. Cl. A61K 31/4725; A61K 31/437; A61K 31/519; A61K 31/5383; C07D 221/04; C07D 471/04; C07D 498/04

(52) U.S. Cl. CPC A61K 31/4725; A61K 31/437; A61K 31/519; A61K 31/5383; C07D 221/04; C07D 471/04; C07D 498/04

(57) ABSTRACT

Described herein are compounds of Formula I and their pharmaceutically acceptable salts, solvates, or stereoisomers thereof, as well as their uses (e.g., for degrading certain proteins such as p300/CBP proteins).

### RELATED APPLICATIONS

This application claims the benefit of and priority to U.S. Provisional Application No. 63/579,540, filed Aug. 30, 2023, the contents of which are incorporated herein by reference in their entireties.

### BACKGROUND

Bifunctional protein degraders are heterobifunctional compounds that simultaneously bind a target protein and an E3 ligase complex, resulting in the transfer of ubiquitin and initiating a process ultimately causing the proteasomal degradation of the target protein. By catalyzing the formation of a ternary complex involving an E3 ligase receptor, a protein of interest, and a small molecule, Bifunctional protein degraders may yield enhanced substrate specificity. There is an ongoing need for selective Bifunctional protein degraders for treating and/or preventing cancer and other diseases responsive to the inhibition or degradation of CBP/p300 proteins. p300 selective degraders can display strong selectivity for p300 over CBP and demonstrate a time-dependent loss of p300, enhancer acetylation, and transcriptional output in cancer cells both in vitro and in vivo with limited toxicity to untransformed cells. Enhanced dependency on p300 is found across numerous cancer lineages.

### SUMMARY

In certain aspects, the present disclosure provides compounds of Formula I:

T-L-C (I),

and pharmaceutically acceptable salts, solvates, or stereoisomers thereof, wherein: C is of Formula I-1

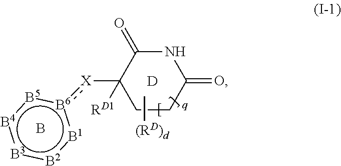

### T is

i) of Formula I-3-i

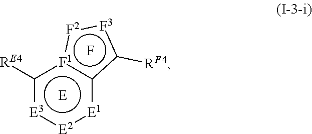

ii) of Formula I-3-ii′

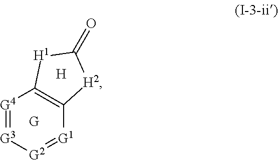

iii) of Formula I-3-iii

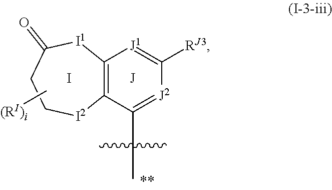

iv) of Formula I-3-iv

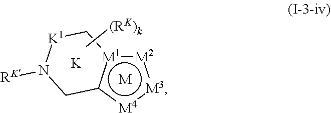

v) of Formula I-3-v

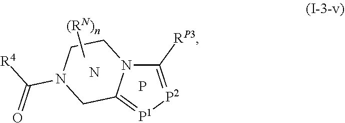

or
vi) of Formula I-3-vi

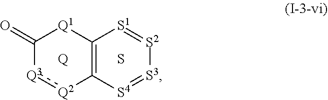

L is of Formula I-2

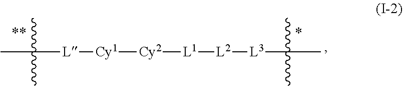

wherein each of the variables in Formulae I-1, I-2, I-3-i, I-3-ii, I-3-iii, I-3-iv, I-3-v, and I-3-vi is described, embodied, and exemplified herein.

In certain aspects, the present disclosure provides pharmaceutical compositions comprising a compound disclosed herein, and a pharmaceutically acceptable excipient.

In certain aspects, the present disclosure provides methods of degrading a protein in a subject or biological sample comprising administering a compound disclosed herein to the subject or contacting the biological sample with the compound disclosed herein.

In certain aspects, the present disclosure provides uses of a compound disclosed herein in the manufacture of a medicament for degrading a protein in a subject or biological sample.

In certain aspects, the present disclosure provides compounds disclosed herein for use in degrading a protein in a subject or biological sample.

In certain aspects, the present disclosure provides methods for treating a disease or disorder.

In certain aspects, the present disclosure provides uses of the compounds disclosed herein in the manufacture of a medicament for treating a disease or disorder.

In certain aspects, the present disclosure provides compounds disclosed herein for treating a disease or disorder.

### DETAILED DESCRIPTION

The present disclosure relates to compounds that show activity in degrading certain proteins (e.g., p300/CBP), and pharmaceutical compositions comprising such compounds.

### Compounds of the Present Disclosure

In certain aspects, the present disclosure provides compounds of Formula I:

T-L-C (I),

and pharmaceutically acceptable salts, solvates, or stereoisomers thereof, wherein:

### C is of Formula I-1

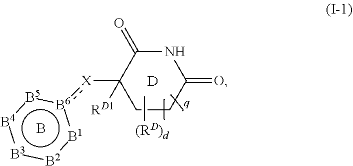

wherein:
[symbol] denotes a single bond or a double bond;
B¹ is CR^{B1}, N, NR^{B1′ 0}, or S;
B² is CR^{B2}, N, NR^{B2′}, O, or S;
B³ is absent, CR^{B3}, or N;
B⁴ is CR^{B4}, N, NR^{B4′}, O, or S;
B⁵ is CR^{B5}, N, NR^{B5′}, O, or S;
B⁶ is C or N;
R^{B1}, R^{B2}, R^{B3}, R^{B4}, and R^{B5} are independently

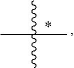

hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}, and one of R^{B1}, R^{B2}, R^{B3}, R^{B4}, and R^{B5} is

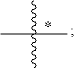

R^{B4} and R^{B5}, together with the carbon atoms to which they are bonded, form 3- to 12-membered heterocyclyl or 5- or 6-membered heteroaryl, wherein the hetercyclyl or heteroaryl is optionally substituted with one or more R^{u}, and is optionally attached to L;
R^{B1′}, R^{B2′}, R^{B4′}, and R^{B5′} are independently hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or—C(═O)NR^{c}R^{d}, wherein the alkyl, carbocyclyl, or heterocyclyl is optionally substituted with one or more R^{u};
X is absent, CR^{X1}, C(R^{X1})₂, NR^{X2} or O, as valency permits;
each R^{X1} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
two R^{X1} together form an oxo; or
two R^{X1}, together with the carbon atom to which they are bonded, form C₃₋₄ carbocyclyl or 3- to 4-membered heterocyclyl, wherein the carbocyclyl or heterocyclyl is optionally substituted with one or more R^{u};
R^{X2} is hydrogen, C₁₋₃ alkyl, C₃₋₄ carbocyclyl, or 3- to 4-membered heterocyclyl, wherein the alkyl, carbocyclyl, or heterocyclyl is optionally substituted with one or more R^{u}; or
R^{X1} and R^{B5}, R^{X1} and R^{B5′}, R^{X2} and R^{B5}, or R^{X2} and R^{B5′}, together with the atoms to which they are bonded, form Ring C, wherein Ring C is optionally substituted C6 aryl, optionally substituted 5- to 6-membered heteroaryl, optionally substituted 5- to 8-membered heterocycle, or optionally substituted C₅₋₈ carbocycle;
R^{D1} is hydrogen, deuterium, halogen, C₁₋₆ alkyl, or C₁₋₆ haloalkyl; or
R^{D1} and one R^{D}, together with the carbon atoms to which they are bonded, form C₃₋₄ carbocycle or 3- to 4-membered heterocycle, wherein the carbocycle or heterocycle is optionally substituted with one or more R;
each R^{D} is independently oxo, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; or
two R^{D}, together with the carbon atom(s) to which they are bonded, form C₃₋₅ carbocyclyl or 3- or 5-membered heterocyclyl, wherein the carbocyclyl or heterocyclyl is optionally substituted with one or more R^{u};
d is an integer from 0 to 6, as valency permits; and
q is an integer from 0 to 2,
wherein when X is NR^{X2} or O, and L″ is absent, then i) R^{X2} and R^{B5}, together with the atoms to which they are bonded, form Ring C; ii) B³ is absent; or iii) R^{D1} and one R^{D}, together with the carbon atoms to which they are bonded, form C₃₋₄ carbocycle or 3- to 4-membered heterocycle, wherein the carbocycle or heterocycle is optionally substituted with one or more R^{u};
T is
i) of Formula I-3-i

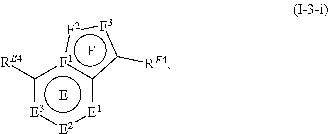

wherein:
E¹ is

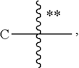

### CR^{E1}, or N;

E² is

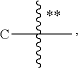

### CR^{E2}, or N;

E³ is

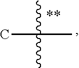

### CR^{E3}, or N;

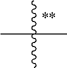

denotes attachment to L;
R^{E1}, R^{E2}, R^{E3}, and R^{E4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
F¹ is C or N;
F² is NR^{F2′} or CR^{F2};
R^{F2′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R^{F2} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—R^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
F³ is CR^{F3}, N, or NR^{F3′};
R^{F3} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —R^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R^{F3′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R^{F4} is —C(═O)NR^{3a}R^{3b} or 5- to 6-membered heteroaryl optionally substituted with one or more R^{u}; and
R^{3a} and R^{3b} are independently hydrogen, C₁₋₆ alkyl, C₂₋₆ alkenyl, C₂₋₆ alkynyl, C₃₋₁₂ carbocyclyl, 3to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R^{3a} and R^{3b}, together with the nitrogen atom to which they are attached, form 3- to 12-membered heterocycle or 5- to 10-membered heteroaryl, wherein the heterocycle or heteroaryl is optionally substituted with one or more R^{u},
ii) of Formula I-3-ii′

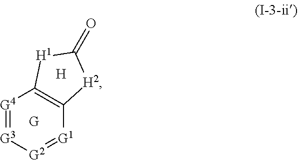

wherein:
G¹ is

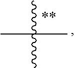

### N, or CR^{G1};

G² is N or CR^{G2};

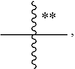

G³ is

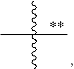

### N, or CR^{G3};

G⁴is

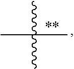

### N, or CR^{G4};

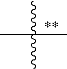

denotes attachment to L;
R^{G1}, R^{G2}, R^{G3}, and R^{G4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
H¹ is C(R^{H1})₂, NR^{H1′} or O;
H² is C(R^{H2})₂, NR^{H2′} or O;
each occurrence of R^{H1} and R^{H2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl wherein the alkyl, alkoxy, alkylamino, alkenyl, alkynyl, carbocyclyl, heterocyclyl, aryl, or heteroaryl is optionally substituted with one or more R^{u}; or
two R^{H1} or two R^{H2}, together with the carbon atom to which they are attached, form C₃₋₆ carbocycle or 3- to 6-membered heterocycle, wherein the carbocycle or heterocycle is optionally substituted with one or more R^{u}; and
R^{H1′} and R^{H2′} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u},
iii) of Formula I-3-iii

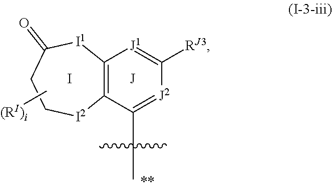

wherein:

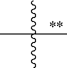

denotes attachment to L;
I¹ is C(R^{I1})₂, NR^{I1′}, or O;
I² is C(R^{I2})₂, NR^{I2′}, or O;
each occurrence of R^{I1} and R^{I2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{I1} or two R^{I2}, together with the carbon atom to which they are attached, form C₃₋₆ carbocycle or 3- to 6-membered heterocycle, wherein the carbocycle or heterocycle is optionally substituted with one or more R^{u};
R^{I1′} and R^{I2′} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
each R is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
i is an integer selected from 0 to 4;
J¹ is N or CR^{J1};
J² is N or CR^{J2}; and
R^{J1}, R^{J2}, and R^{J3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
iv) of Formula I-3-iv

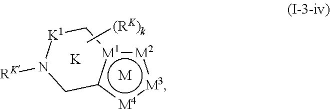

wherein:
M¹ is N or C;
M² is

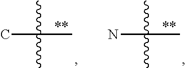

### N, or CR^{M2};

M³ is

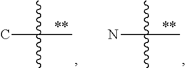

### N, or CR^{M3};

M⁴ is

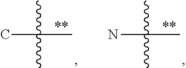

### N, or CR^{M4};

wherein

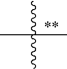

denotes attachment to L; and at least one of M¹, M², M³, and M⁴ is N or

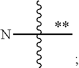 [SMILES: *N]

R^{M2}, R^{M3}, and R^{M4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R^{K′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
K¹ is —C(═O)— or —C(RK¹)₂—;
each R^{K1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
each R^{K} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
k is an integer selected from 0 to 4;
v) of Formula I-3-v

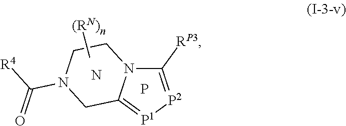

wherein:
P¹ is

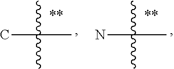

### N, or CR^{P1}

P² is

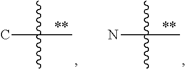

### N or CR^{P2}.

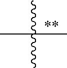

denotes attachment to L;
each R^{N} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
n is an integer selected from 0 to 6;
R^{P1}, R^{P2}, and R^{P3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
R⁴ is —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
or
vi) of Formula I-3-vi

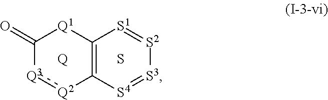

wherein:
Q¹ is NR^{Q1′}, C(R^{Q1})₂, or O;
R^{Q1′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
each R^{Q1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{Q1}, together with the carbon atom to which they are attached, form C₃₋₆ carbocycle or 3- to 6-membered heterocycle, wherein the carbocycle or heterocycle is optionally substituted with one or more R^{u};
when [symbol] is a double bond, then Q² is N or CR^{Q2}; and Q³ is N or CR^{Q3};
when [symbol] is a single bond, then Q² is NR^{Q2′}, C(R^{Q2})₂, or O; Q³ is NR^{Q3′}, C(R^{Q3})₂, or O;
R^{Q2′} and R^{Q3′} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
each occurrence of R^{Q2} and R^{Q3} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{Q2} or two R^{Q3}, together with the carbon atom to which they are attached, form C₃₋₆ carbocycle or 3- to 6-membered heterocycle, wherein the carbocycle or heterocycle is optionally substituted with one or more R^{u};
S¹ is CR^{S1} or N;
S² is

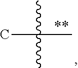

### CR^{S2}, or N;

S³ is

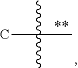

### CR^{S3}, or N;

S⁴ is

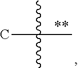

### CR^{S4}, or N;

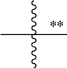

denotes attachment to L; and
R^{S1}, R^{S2}, R^{S3}, and R^{S4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
L is of Formula I-2

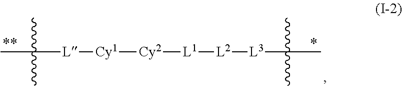

wherein:
* denotes attachment to T and * denotes attachment to C;
L″ is absent; or
L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u};
R^{L} is hydrogen, C₁₋₆ alkyl, C₁₋₆ haloalkyl, C₃₋₆ carbocyclyl, or 3- to 6-membered heterocyclyl, wherein the alkyl, haloalkyl, carbocyclyl, or heterocyclyl is optionally substituted with one or more R^{u};
wherein:
i) when L″ is C₁₋₃ alkylene, —C(═O)—, —C(═O)N(R^{L})—, —C(═O)O—, —N(R^{L})—, —O—, —S—, or —S(═O)₂, wherein the alkylene is optionally substituted with one or more R^{u}, then Cy¹ is optionally substituted C6 arylene or 5- to 6-membered heteroarylene;
ii) when L″ is absent, then
   Cy¹ is

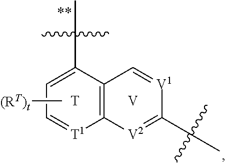

wherein:
* denotes attachment to T;
T¹ is CR^{T1} or N;
V¹ is CR^{V1} or N;
V² is CRV² or N;
R^{T1}, R^{V1}, and RV² are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R;
each R^{T} is independently halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
t is an integer selected from 0 to 6, as valency permits,
wherein R^{T} may be present on either Ring T or Ring V;
or
Cy¹ is

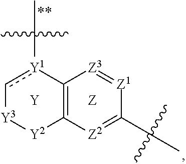

wherein:
when [symbol] is single bond, Y¹ is N or CR^{Y1}; when [symbol] is double bond, Y¹ is C;
* denotes attachment to T;
R^{Y1} is hydrogen, halogen, —CN, —NO₂, —OH, —NH₂, C₁₋₆ alkyl, C₁₋₆ alkoxy, C₁₋₆ alkylamino, C3-4 carbocyclyl, or 3- to 4-membered heterocyclyl, wherein the alkyl, alkoxy, alkylamino, carbocyclyl, or heterocyclyl is optionally substituted with one or more R^{u};
Y² is C(R^{Y2})₂, O, or N(R^{Y2′})
each R^{Y2} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; or
two R^{Y2} together form an oxo;
R^{Y2′} is hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
Y³ is C(R^{Y3})₂ or N(R^{Y3′});
each R^{Y3} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}
R^{Y2′} is hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
Z¹ is CR^{Z1} or N;
Z² is CR^{Z2} or N;
Z³ is CR^{Z3} or N;
R^{Z1}, R^{Z2}, R^{Z3} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
Cy² is 5- to 6-membered heteroarylene optionally substituted with one or more R^{u};
L¹ is —C(═O)NH— or 5- to 6-membered heteroaryl optionally substituted with one or more R^{u};
L² is —C(R^{L2})₂—, wherein
each R^{L2} is independently hydrogen, deuterium, C₁₋₃ alkyl, or C₁₋₃ haloalkyl; or
two geminal R^{L2}, together with the carbon atom to which they are bonded, form C₃₋₅ carbocyclyl or 3- to 5-membered heterocyclyl, wherein the carbocyclyl or heterocyclyl is optionally substituted with one or more R;
and
L³ is E-ethenylene, ethynylene, C₆ arylene, or 5- to 6-membered heteroarylene, wherein the E-ethenylene, arylene, or heteroarylene is optionally substituted with one or more R^{u};
wherein:
each R^{u}; is independently oxo, deuterium, halogen, —CN, —NO₂, —OH, —NH₂, C₁₋₆ alkyl, C₁₋₆ alkoxy, C₁₋₆ alkylamino, C₃₋₄ carbocyclyl, 3- to 4-membered heterocyclyl, —C(═O)R^{a}, or —S(═O)₂R^{a};
each R^{a} is independently C₁₋₆ alkyl, C₃₋₆ carbocyclyl, or 3- to 6-membered heterocyclyl;
each R^{b} is independently hydrogen, C₁₋₆ alkyl, C₃₋₆ carbocyclyl, or 3- to 6-membered heterocyclyl; and
each R^{c} and R^{d} is independently hydrogen, C₁₋₆ alkyl, C₃₋₆ carbocyclyl, or 3- to 6-membered heterocyclyl; or
R^{c} and R^{d}, together with the nitrogen atom to which they are attached, form 3- to 6-membered heterocyclyl,
wherein each occurrence of R^{a}, R^{b}, R^{c}, and R^{d} is independently and optionally substituted with one or more R^{Z}; and
each R^{Z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl.

In certain embodiments, the present disclosure provides compounds of Formula I:

T-L-C (I),

and pharmaceutically acceptable salts, solvates, or stereoisomers thereof, wherein:
C is of Formula I-1

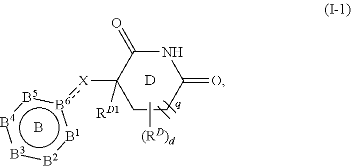

wherein:
[symbol] denotes a single bond or a double bond;
B¹ is CRB, N, NR^{B1′}, O, or S;
B² is CR^{B2}, N, NR^{B2′}, O, or S;
B³ is absent, CR^{B3}, or N;
B⁴ is CR^{B4}, N, NR^{B4′}, O, or S;
B⁵ is CR^{B5}, N, NR^{B5′}, O, or S;
B⁶ is C or N;
R^{B1}, R^{B2}, R^{B3}, R^{B4}, and R^{B5} are independently

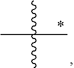

hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}, and one of RB, R^{B2}, R^{B3}, R^{B4}, and R^{B5} is

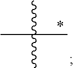

or
R^{B4} and R^{B5}, together with the carbon atoms to which they are bonded, form 3- to 12-membered heterocyclyl optionally substituted with one or more R^{u};
R^{B1′}, R^{B2′}, R^{B4′}, and R^{B5′} are independently hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
X is absent, CR^{X1}, C(R^{X1})₂, NR^{X2} or O, as valency permits;
each R^{X1} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
two R^{X1} together form an oxo; or
two R^{X1}, together with the carbon atom to which they are bonded, form C₃₋₄ carbocyclyl or 3- to 4-membered heterocyclyl, wherein the carbocyclyl or heterocyclyl is optionally substituted with one or more R;
R^{X2} is hydrogen, C₁₋₃ alkyl, C₃₋₄ carbocyclyl, or 3- to 4-membered heterocyclyl, wherein the alkyl, carbocyclyl, or heterocyclyl is optionally substituted with one or more R^{u}; or
R^{X1} and R^{B5}, R^{X1} and R^{B5′}, R^{X2} and R^{B5}, or R^{X2} and R^{B5′}, together with the atoms to which they are bonded, form Ring C, wherein Ring C is optionally substituted C₆ aryl, optionally substituted 5- to 6-membered heteroaryl, optionally substituted 5- to 6-membered heterocycle, or optionally substituted C₅₋₆ carbocycle;
R^{D1} is hydrogen, deuterium, halogen, C₁₋₆ alkyl, or C₁₋₆ haloalkyl; or
R^{D1} and one R^{D}, together with the carbon atoms to which they are bonded, form C₃₋₄ carbocycle or 3- to 4-membered heterocycle, wherein the carbocycle or heterocycle is optionally substituted with one or more R^{u};
each R^{D} is independently halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; or
two R^{D}, together with the carbon atom(s) to which they are bonded, form C₃₋₅ carbocyclyl or 3- or 5-membered heterocyclyl, wherein the carbocyclyl or heterocyclyl is optionally substituted with one or more R^{u};
d is an integer from 0 to 6, as valency permits; and
q is an integer from 0 to 2,
wherein when X is NR^{X2} or O, and L″ is absent, then i) R^{X2} and R^{B5}, together with the atoms to which they are bonded, form Ring C; ii) B³ is absent; or iii) R^{D1} and one R^{D}, together with the carbon atoms to which they are bonded, form C₃₋₄ carbocycle or 3- to 4-membered heterocycle, wherein the carbocyclyl or heterocyclyl is optionally substituted with one or more R^{u};
T is
i) of Formula I-3-i

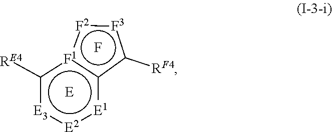

wherein:
E¹ is

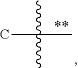

### CR^{E1}, or N;

E²is

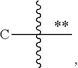

### CR^{E2}, or N;

E³ is

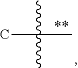

### CR^{E3}, or N;

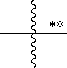

denotes attachment to L;
R^{E1}, R^{E2}, R^{E3}, and R^{E4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
F¹ is C or N;
F² is NR^{F2′} or CR^{F2};
R^{F2′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R^{F2} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —R^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
F³ is CR^{F3}, N, or NR^{F3′};
R^{F3} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R;
R^{F3′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R^{F4} is —C(═O)NR^{3a}R^{3b} or 5- to 6-membered heteroaryl optionally substituted with one or more R^{u}; and
R^{3a} and R^{3b} are independently hydrogen, C₁₋₆ alkyl, C₂₋₆ alkenyl, C₂₋₆ alkynyl, C₃₋₁₂ carbocyclyl, 3to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R; or
R^{3a} and R^{3b}, together with the nitrogen atom to which they are attached, form 3- to 12-membered heterocycle or 5- to 10-membered heteroaryl, wherein the heterocycle or heteroaryl is optionally substituted with one or more R^{u};
ii) of Formula I-3-ii

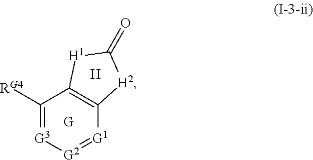

wherein:
G¹ is

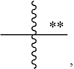

### N, or CR^{G1};

G² is

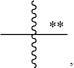

### N, or CR^{G2};

G³ is

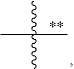

### N, or CR^{G3};

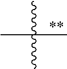

denotes attachment to L;
R^{G1}, R^{G2}, R^{G3}, and R^{G4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
H¹ is C(R^{H1})₂, NR^{H1′} or O;
H² is C(R^{H2})₂, NR^{H2′} or O;
each occurrence of R^{H1} and R^{H2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl wherein the alkyl, alkoxy, alkylamino, alkenyl, alkynyl, carbocyclyl, heterocyclyl, aryl, or heteroaryl is optionally substituted with one or more R^{u}; or
two R^{H1} or two R^{H2}, together with the carbon atom to which they are attached, form C₃₋₆ carbocycle or 3- to 6-membered heterocycle, wherein the carbocycle or heterocycle is optionally substituted with one or more R^{u}; and
RH^{1′} and R^{H2′} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
iii) of Formula I-3-iii

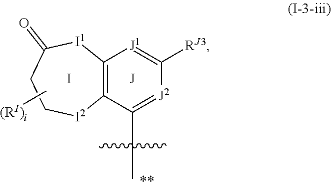

wherein:

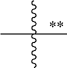

denotes attachment to L;
I¹ is C(R^{I1})₂, NR^{H′}, or O;
I² is C(R^{I2})₂, NR^{I2′}, or O;
each occurrence of R^{I1} and R^{I2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or two R^{I1} or two R^{I2}, together with the carbon atom to which they are attached, form C₃₋₆ carbocycle or 3- to 6-membered heterocycle, wherein the carbocycle or heterocycle is optionally substituted with one or more R;
R^{I1′} and R^{I2′} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R;
each R¹ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
i is an integer selected from 0 to 4;
J¹ is N or CR^{J1};
J² is N or CR^{J2}; and
R^{J1}, R^{J2}, and R^{J3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
iv) of Formula I-3-iv

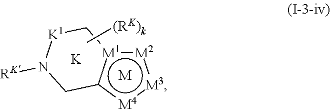

wherein:
M¹ is N or C;
M² is

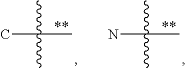

### N, or CR^{M2};

M³ is

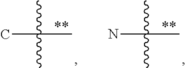

### N, or CR^{M3};

M⁴ is

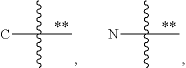

### N, or CR^{M4}.

wherein

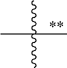

denotes attachment to L; and at least one of M¹, M², M³, and M⁴ is N or

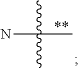 [SMILES: *N]

R^{M2}, R^{M3}, and or R^{M4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R^{K′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
K¹ is —C(═O)— or —C(R^{K1})₂—;
each R^{K1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}
each R^{K} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
k is an integer selected from 0 to 4;
v) of Formula I-3-v

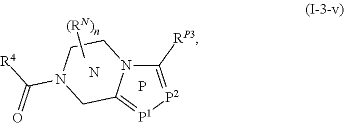

wherein:
P¹ is

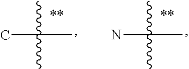

### N, or CR^{P1};

P² is

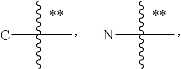

### N or CR^{P2};

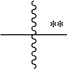

denotes attachment to L;
each R^{N} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
n is an integer selected from 0 to 6;
R^{P1}, R^{P2}, and R^{P3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
R⁴ is —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
or
vi) of Formula I-3-vi

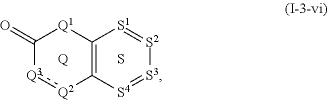

wherein:
Q¹ is NR^{Q1′}, C(R^{Q1})₂, or O;
R^{Q1′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
each R^{Q1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{Q1}, together with the carbon atom to which they are attached, form C₃₋₆ carbocycle or 3- to 6-membered heterocycle, wherein the carbocycle or heterocycle is optionally substituted with one or more Rfu,
when [symbol] is a double bond, then Q² is N or CR^{Q2}; and Q³ is N or CR^{Q3};
when [symbol] is a single bond, then Q² is NR^{Q2′}, C(R^{Q2})₂, or O; Q³ is NR^{3′}, C(R^{Q3})₂, or O;
R^{Q2′} and R^{Q3′} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
each occurrence of R^{Q2} and R^{Q3} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{Q2} or two RQ, together with the carbon atom to which they are attached, form C₃₋₆ carbocycle or 3- to 6-membered heterocycle, wherein the carbocycle or heterocycle is optionally substituted with one or more R;
S¹ is CR^{S1} or N;
S² is

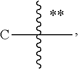

### CR^{S2}, or N;

S³ is

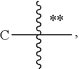

### CR^{S3}, or N;

S⁴ is

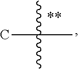

### CR^{S4}, or N;

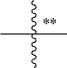

denotes attachment to L; and
R^{S1}, R^{S2}, R^{S3}, and R^{S4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
L is of Formula I-2

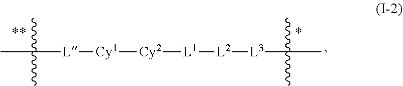

wherein:
* denotes attachment to T and * denotes attachment to C;
L″ is absent; or
L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u};
wherein:
i) when L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u}, then Cy¹ is optionally substituted C₆ arylene or 5- to 6-membered heteroarylene;
ii) when L″ is absent, then
   Cy¹ is

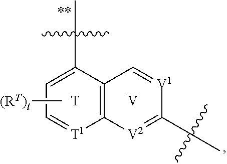

wherein:
** denotes attachment to T;
T¹ is CR^{T1} or N;
V¹ is CR^{V1} or N;
V² is CRV² or N;
R^{T1}, R^{V1}, and RV² are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R;
each R^{T} is independently halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
t is an integer selected from 0 to 6, as valency permits;
wherein R^{T} may be present on either Ring T or Ring V;
or
Cy¹ is

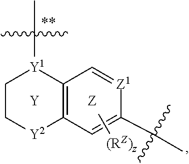

wherein:
Y¹ is N or CR^{Y1};
** denotes attachment to T;
R^{Y1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
Y² is C(R^{Y2})₂, O, or N(R^{Y2′});
each R^{Y2} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}
R^{Y2′} is hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
Z¹ is CR^{Z1} or N;
R^{Z1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, 3- to 4-membered heterocyclyl, wherein the alkyl, alkoxy, alkylamino, carbocyclyl, or heterocyclyl is optionally substituted with one or more R^{u} each R^{Z} is independently oxo, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}z is an integer selected from 0 to 6, as valency permits,
wherein R^{Z} may be present on either Ring Y or Ring Z;
Cy² is 5- to 6-membered heteroarylene optionally substituted with one or more R^{u};
L¹ is —C(═O)NH— or 5- to 6-membered heteroaryl optionally substituted with one or more R^{u};
L² is C(R^{L2})₂, wherein
each R^{L2} is independently hydrogen, C₁₋₃ alkyl, or C₁₋₃ haloalkyl; or
two geminal R^{L2}, together with the carbon atom to which they are bonded, form C₃₋₅ carbocyclyl or 3- to 5-membered heterocyclyl, wherein the carbocyclyl or heterocyclyl is optionally substituted with one or more R^{u}; and
L³ is E-ethenylene, ethynylene, C₆ arylene, or 5- to 6-membered heteroarylene, wherein the E-ethenylene, arylene, or heteroarylene is optionally substituted with one or more R^{u};
wherein:
each R^{u}; is independently oxo, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl,
each R^{a} is independently C₁₋₆ alkyl, C₃₋₆ carbocyclyl, or 3- to 6-membered heterocyclyl;
each R^{b} is independently hydrogen, C₁₋₆ alkyl, C₃₋₆ carbocyclyl, or 3- to 6-membered heterocyclyl; and
each R^{c} and R^{d} is independently hydrogen, C₁₋₆ alkyl, C₃₋₆ carbocyclyl, or 3- to 6-membered heterocyclyl; or
R^{c} and R^{d}, together with the nitrogen atom to which they are attached, form 3- to 6-membered heterocyclyl,
wherein each occurrence of R^{a}, R^{b}, R^{c}, and R^{d} is independently and optionally substituted with one or more R^{Z}; and
each R^{Z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl.

In certain embodiments, C is of Formula I-1

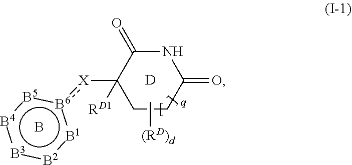

wherein
[symbol] denotes a single bond or a double bond;
B¹ is CR^{B1}, N, NR^{B1′}, O, or S;
B² is CR^{B2}, N, NR^{B2′}, O, or S;
B³ is absent, CR^{B3}, or N;
B⁴ is CR^{B4}, N, NR^{B4′}, O, or S;
B⁵ is CR^{B5}, N, NR^{B5′}, O, or S;
B⁶ is C or N;
R^{B1}, R^{B2}, R^{B3}, R^{B4}, and R^{B5} are independently

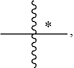

hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}, and one of R^{B1}, R^{B2}, R^{B3}, R^{B4}, and R^{B5} is

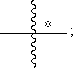

R^{B4} and R^{B5}, together with the carbon atoms to which they are bonded, form 3- to 12-membered heterocyclyl or 5- or 6-membered heteroaryl, wherein the hetercyclyl or heteroaryl is optionally substituted with one or more R^{u}, and is optionally attached to L;
R^{B1′}, R^{B2′}, R^{B4′}, and R^{B5′} are independently hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
X is absent, CR^{X1}, C(R^{X1})₂, NR^{X2} or O, as valency permits;
each R^{X1} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
two R^{X1} together form an oxo; or
two R^{X1}, together with the carbon atom to which they are bonded, form C₃₋₄ carbocyclyl or 3- to 4-membered heterocyclyl, wherein the carbocyclyl or heterocyclyl is optionally substituted with one or more R^{u};
R^{X2} is hydrogen, C₁₋₃ alkyl, C₃₋₄ carbocyclyl, or 3- to 4-membered heterocyclyl, wherein the alkyl, carbocyclyl, or heterocyclyl is optionally substituted with one or more R^{u}; or
R^{X1} and R^{B5}, R^{X1} and R^{B5′}, R^{X2} and R^{B5}, or R^{X2} and R^{B5′}, together with the atoms to which they are bonded, form Ring C, wherein Ring C is optionally substituted C₆ aryl, optionally substituted 5- to 6-membered heteroaryl, optionally substituted 5- to 8-membered heterocycle, or optionally substituted C₅₋₈ carbocycle;
R^{D1} is hydrogen, deuterium, halogen, C₁₋₆ alkyl, or C₁₋₆ haloalkyl; or
R^{D1} and one R^{D}, together with the carbon atoms to which they are bonded, form C₃₋₄ carbocycle or 3- to 4-membered heterocycle, wherein the carbocycle or heterocycle is optionally substituted with one or more R^{u};
each R^{D} is independently oxo, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; or
two R^{D}, together with the carbon atom(s) to which they are bonded, form C₃₋₅ carbocyclyl or 3- or 5-membered heterocyclyl, wherein the carbocyclyl or heterocyclyl is optionally substituted with one or more R;
d is an integer from 0 to 6, as valency permits; and
q is an integer from 0 to 2,
wherein when X is NR^{X2} or O, and L″ is absent, then i) R^{X2} and R^{B5}, together with the atoms to which they are bonded, form Ring C; ii) B³ is absent; or iii) R^{D1} and one R^{D}, together with the carbon atoms to which they are bonded, form C₃₋₄ carbocycle or 3- to 4-membered heterocycle, wherein the carbocycle or heterocycle is optionally substituted with one or more R.

In certain embodiments,

### C is of Formula I-1

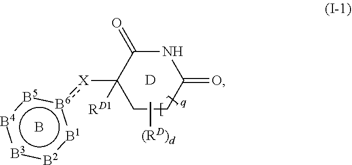

wherein
[symbol] denotes a single bond or a double bond;
B¹ is CRB, N, NR^{B1′} O, or S;
B² is CR^{B2}, N, NR^{B2′} O, or S;
B³ is absent, CR^{B3}, or N;
B⁴ is CR^{B4}, N, NR^{B4′} O, or S;
B⁵ is CR^{B5}, N, NR^{B5′} O, or S;
B⁶ is C or N;
R^{B1}, R^{B2}, R^{B3}, R^{B4}, and R^{B5} are independently

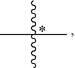

hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted
with one or more R^{u}, and one of R^{B1}, R^{B2}, R^{B3}, R^{B4}, and R^{B5} is

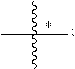

or
R^{B4} and R^{B5}, together with the carbon atoms to which they are bonded, form 3- to 12-membered heterocyclyl optionally substituted with one or more R^{u};
R^{B1′}, R^{B2′}, R^{B4′}, and R^{B5′} are independently hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
X is absent, CR^{X}l, C(R^{X1})₂, NR^{X2} or O, as valency permits;
each R^{X1} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
two R^{X1} together form an oxo; or
two R^{X1}, together with the carbon atom to which they are bonded, form C₃₋₄ carbocyclyl or 3- to 4-membered heterocyclyl, wherein the carbocyclyl or heterocyclyl is optionally substituted with one or more R^{u};
R^{X2} is hydrogen, C₁₋₃ alkyl, C₃₋₄ carbocyclyl, or 3- to 4-membered heterocyclyl, wherein the alkyl, carbocyclyl, or heterocyclyl is optionally substituted with one or more R^{u}; or
R^{X1} and R^{B5}, R^{X1} and R^{B5′}, R^{X2} and R^{B5}, or R^{X2} and R^{B5′}, together with the atoms to which they are bonded, form Ring C, wherein Ring C is optionally substituted C₆ aryl, optionally substituted 5- to 6-membered heteroaryl, optionally substituted 5- to 6-membered heterocycle, or optionally substituted C₅₋₆ carbocycle;
R^{D1} is hydrogen, deuterium, halogen, C₁₋₆ alkyl, or C₁₋₆ haloalkyl; or
R^{D1} and one R^{D}, together with the carbon atoms to which they are bonded, form C₃₋₄ carbocycle or 3- to 4-membered heterocycle, wherein the carbocyclyl or heterocyclyl is optionally substituted with one or more R^{u};
each R^{D} is independently halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; or
two R^{D}, together with the carbon atom(s) to which they are bonded, form C₃₋₅ carbocyclyl or 3- or 5-membered heterocyclyl, wherein the carbocyclyl or heterocyclyl is optionally substituted with one or more R;
d is an integer from 0 to 6, as valency permits; and
q is an integer from 0 to 2,
wherein when X is NR^{X2} or O, and L″ is absent, then i) R^{X2} and R^{B5}, together with the atoms to which they are bonded, form Ring C; ii) B³ is absent; or iii) R^{D1} and one R^{D}, together with the carbon atoms to which they are bonded, form C₃₋₄ carbocycle or 3- to 4-membered heterocycle, wherein the carbocycle or heterocycle is optionally substituted with one or more R^{u}.

In certain embodiments, X is absent, CR^{X1}, C(R^{X1})₂, NR^{X2} or O, as valency permits.

In certain embodiments, X is absent.

In certain embodiments, X is CRXi, as valency permits.

In certain embodiments, X is C(R^{X1})₂.

In certain embodiments, X is NR^{X2}, and L″ is absent, and i) R^{X2} and R^{B5}, together with the atoms to which they are bonded, form Ring C.

In certain embodiments, X is NR^{X2}, and L″ is absent, and ii) B³ is absent.

In certain embodiments, X is NR^{X2}, and L″ is absent, and iii) R^{D1} and one R^{D} together with the carbon atoms to which they are bonded, form C₃₋₄ carbocycle or 3- to 4-membered heterocycle, wherein the carbocycle or heterocycle is optionally substituted with one or more R^{u}.

In certain embodiments, X is O, and L″ is absent, and i) R^{X2} and R^{B5}, together with the atoms to which they are bonded, form Ring C.

In certain embodiments, X is O, and L″ is absent, and ii) B³ is absent.

In certain embodiments, X is O, and L″ is absent, and iii) R^{D1} and one R^{D}, together with the carbon atoms to which they are bonded, form C₃₋₄ carbocycle or 3- to 4-membered heterocycle, wherein the carbocycle or heterocycle is optionally substituted with one or more R^{u}.

In certain embodiments, each R^{X1} is independently hydrogen, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R^{u}.

In certain embodiments, two R^{X1} together form an oxo.

In certain embodiments, two R^{X1}, together with the carbon atom to which they are bonded, form C₃₋₄ carbocyclyl (e.g., cyclopropyl (C₃), cyclopropenyl (C₃), cyclobutyl (C₄), or cyclobutenyl (C₄)) or 3- to 4-membered heterocyclyl (e.g., heterocyclyl comprising one 3- to 4-membered rings and 1 heteroatom selected from N, O, and S), wherein the carbocyclyl or heterocyclyl is optionally substituted with one or more R^{u}.

In certain embodiments, R^{X2} is hydrogen, C₁₋₃ alkyl (e.g., methyl (C₁), ethyl (C₂), n-propyl (C₃), or i-propyl (C₃)), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, carbocyclyl, or heterocyclyl is optionally substituted with one or more R^{u}.

In certain embodiments, B¹ is CR^{B1}, N, NR^{B1′} O, or S. In certain embodiments, B¹ is CR^{B1}. In certain embodiments, B¹ is N. In certain embodiments, B¹ is NR^{B′}. In certain embodiments, B¹ is O.

In certain embodiments, B² is CR^{B2}, N, NR^{B2′} O, or S. In certain embodiments, B² is CR^{B2}. In certain embodiments, B² is N. In certain embodiments, B² is NR^{B2′}. In certain embodiments, B² is O.

In certain embodiments, B³ is absent, CR^{B3}, or N. In certain embodiments, B³ is absent. In certain embodiments, B³ is CR^{B3}. In certain embodiments, B³ is N.

In certain embodiments, B⁴ is CR^{B4}, N, NR^{B4′} O, or S. In certain embodiments, B⁴ is CR^{B4}. In certain embodiments, B⁴ is N. In certain embodiments, B⁴ is NR^{B4′}. In certain embodiments, B⁴ is O.

In certain embodiments, B⁵ is CRB, N, NRBs' O, or S. In certain embodiments, B⁵ is CR^{B5}. In certain embodiments, B⁵ is N. In certain embodiments, B⁵ is NR^{B5′}. In certain embodiments, B⁵ is O.

In certain embodiments, B⁶ is C or N. In certain embodiments, B⁶ is C. In certain embodiments, B⁶ is N.

In certain embodiments, C is of Formula I-1-i or I-1-ii

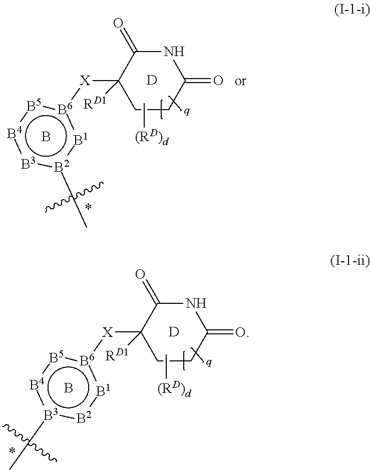

In certain embodiments, C is of Formula I-1-i

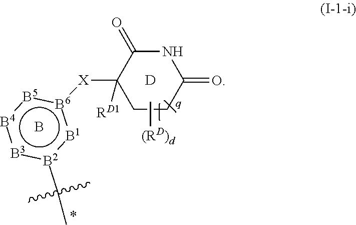

In certain embodiments, C is of Formula I-1-i″

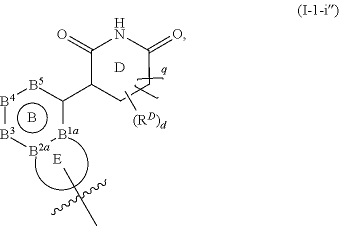

wherein:
Ring E is optionally substituted 5- or 6-membered heteroaryl or optionally substituted 5- or 6-membered heterocycle, and
B^{1a} and B²a are independently C or N.

In certain embodiments, C is of Formula T-1-i″-1

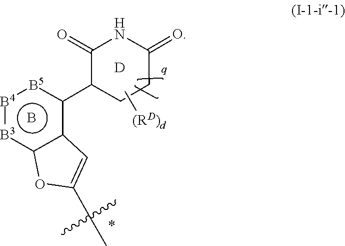

In certain embodiments, none of B¹, B³, B⁴, and B⁵ is N.

In certain embodiments, one of B¹ and B⁵ is N.

In certain embodiments, R^{B}i, R^{B3}, R^{B4}, and R^{B5}, R^{B}i, R^{B3}, and R^{B4}, R^{B3}, R^{B4}, and R^{B5} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R.

In certain embodiments, R^{B1}, R^{B3}, R^{B4}, and R^{B5}, R^{B1}, R^{B3}, and R^{B4}, R^{B3}, R^{B4}, and R^{B5} are independently hydrogen or C₁₋₆ alkyl.

In certain embodiments, R^{B4} and R^{B5}, together with the carbon atoms to which they are bonded, form 3- to 12-membered heterocyclyl (e.g., heterocyclyl comprising one or two rings and 1-5 heteroatoms selected from N, O, and S) optionally substituted with one or more R^{u}.

In certain embodiments, R^{B4} and R^{B5}, together with the carbon atoms to which they are bonded, form 3- to 12-membered heterocyclyl or 5- or 6-membered heteroaryl, wherein the hetercyclyl or heteroaryl is optionally substituted with one or more R^{u}, and is optionally attached to L.

In certain embodiments, R^{X1} and R^{B5}, R^{X1} and R^{B5′}, R^{X2} and R^{B5}, or R^{X2} and RBs' together with the atoms to which they are bonded, form Ring C, wherein Ring C is optionally substituted C₆ aryl or optionally substituted 5-membered heteroaryl.

In certain embodiments, R^{X1} and R^{B5} or R″l and R^{B5′}, together with the atoms to which they are bonded, form Ring C, wherein Ring C is optionally substituted C₅₋₆ carbocyclyl or optionally substituted 5- or 6-membered heterocyclyl.

In certain embodiments, Ring C is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —R^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, and —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, Ring C is optionally substituted with one or more R^{u}.

In certain embodiments, Ring C is optionally substituted with one or more R^{B7}, R^{B8}R^{B7′}, R^{B9}, R^{B10}, or R^{B11}.

In certain embodiments, R^{u}; is R^{B7}, R^{B8}, R^{B7′}, R^{B8′}, R^{B9}, R^{B10}, or R^{B11}

In certain embodiments, C is of Formula I-1-iii

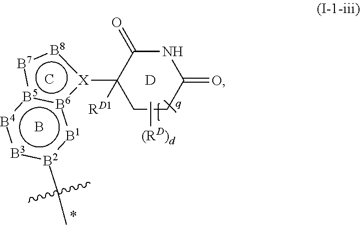

wherein:
B^{5a} is C or N;
B⁷ is CR^{B7}, N, NR^{B7′}, O, or S;
B⁸ is CR^{B8}, N, NR^{B8′}, O, or S;
R^{B7} and R^{B8} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; and R^{B7′} and R^{B8′} are independently hydrogen, C₁₋₆ alkyl, C₁₋₆ alkoxy, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u} wherein at least one of B^{5a}, B⁶, B⁷, B⁸, and X is N, NR^{B7′} NR^{B8′}, O, or S.

In certain embodiments, B^{5a} is C or N.

In certain embodiments, B⁵ is Bsa

In certain embodiments, B⁷ is CR^{B7}, N, NR^{B7′}, O, or S.

In certain embodiments, B⁸ is CR^{B8}, N, NR^{B8′}, O, or S.

In certain embodiments, at least one of B⁷ and B⁸ is N.

In certain embodiments, B⁷ is NR^{B7′}, and B⁸ is N.

In certain embodiments, B⁷ is N and X is N.

In certain embodiments, B⁸ is N and X is N.

In certain embodiments, C is of Formula I-1-iii-1, I-1-iii-2, I-1-iii-3, or I-1-iii-4

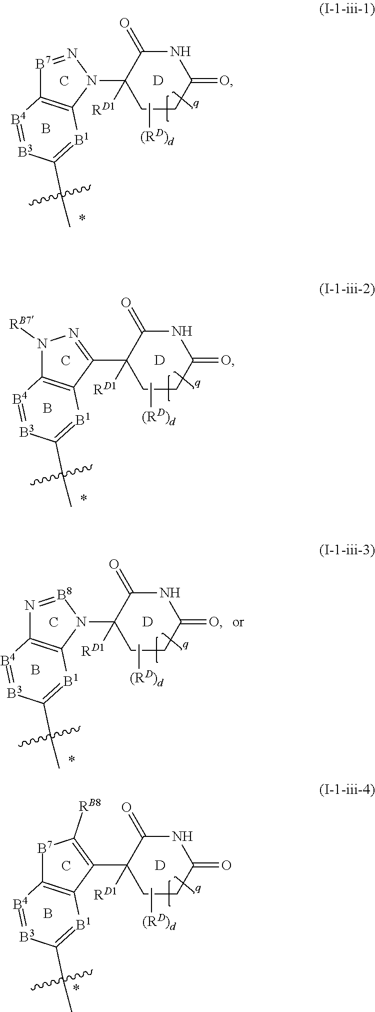

wherein when C is of Formula I-1-iii-4, B⁷ is O or S.

In certain embodiments, R^{B7} and R^{B8} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R^{u}.

In certain embodiments, R^{B8} are independently hydrogen or C₁₋₆ alkyl.

In certain embodiments, R^{B7′} and R^{B8′} are independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₄ carbocyclyl (e.g., cyclopropyl (C₃), cyclopropenyl (C₃), cyclobutyl (C₄), or cyclobutenyl (C₄)), 3- to 4-membered heterocyclyl (e.g., heterocyclyl comprising one 3- to 4-membered rings and 1 heteroatom selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, R^{B7′} and R^{B8′} are independently hydrogen or C₁₋₆ alkyl.

In certain embodiments, R^{B}i, R^{B3}, and R^{B4} are independently hydrogen, halogen (e.g., —F, —Cl, —Br, or —I), —CN, —NO₂, —OH, —NH₂, C₁₋₆ alkyl (e.g., methyl C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R^{u}.

In certain embodiments, each of R^{B}i, R^{B3}, and R^{B4} is hydrogen.

In certain embodiments, C is of Formula I-1-iv

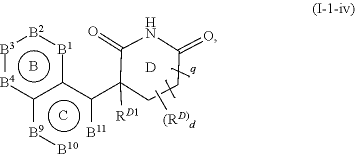

wherein:
B⁹ is CR^{B9} or N;
B¹⁰ is CR^{B10} or N;
B¹¹ is CR^{B11} or N; and
R^{B9}, R^{B10}, and R^{B11} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}.

In certain embodiments, C is of Formula I-1-iv-1, I-1-iv-2, or I-1-iv-3

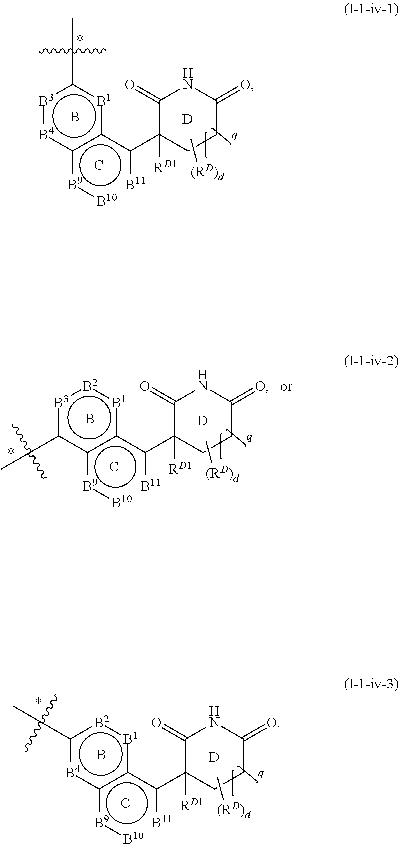

In certain embodiments, none of R^{B9}, R^{B10}, and R^{B11} is N.

In certain embodiments, one of R^{B9}, R^{B10}, and R^{B11} is N.

In certain embodiments, two of R^{B9}, R^{B10}, and R^{B11} are N.

In certain embodiments, three of R^{B9}, R^{B10}, and R^{B11} are N.

In certain embodiments, R^{B9}, R^{B10}, and R^{B11}, when applicable, are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R^{u}.

In certain embodiments, each of R^{B9}, R^{B10}, and R^{B11}, when applicable, is hydrogen.

In certain embodiments, none of R^{B1}, R^{B2}, and R^{B3} or none of R^{B1}, R^{B3}, and R^{B4} is N.

In certain embodiments, one of R^{B1}, R^{B2}, and R^{B3} or one of R^{B1}, R^{B3}, and R^{B4} is N.

In certain embodiments, two of R^{B1}, R^{B2}, and R^{B3} or two of R^{B1}, R^{B3}, and R^{B4} are N.

In certain embodiments, three of R^{B1}, R^{B2}, and R^{B3} or three of R^{B1}, R^{B3}, and R^{B4} are N.

In certain embodiments, R^{B1}, R^{B2}, and R^{B3} or R^{B1}, R^{B3}, and R^{B4}, when applicable, are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R.

In certain embodiments, each of R^{B}i, R^{B2}, and R^{B3}, or each of R^{B}i, R^{B2}, and R^{B3} when applicable, is hydrogen.

In certain embodiments, R^{D1} is hydrogen, deuterium, or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}.

In certain embodiments, R^{D1} is hydrogen.

In certain embodiments, each R^{D} is independently oxo, hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R.

In certain embodiments, two R^{D}, together with the carbon atom(s) to which they are bonded, form C₃₋₅ carbocyclyl (e.g., cyclopropyl (C₃), cyclopropenyl (C₃), cyclobutyl (C₄), cyclobutenyl (C₄), cyclopentyl (C₅), or cyclopentenyl (C₅)) or 3- or 5-membered heterocyclyl (e.g., heterocyclyl comprising one or two 3- to 5-membered rings and 1-3 heteroatoms selected from N, 0, and S), wherein the carbocyclyl or heterocyclyl is optionally substituted with one or more R^{u}.

In certain embodiments, d is an integer from 0 to 6, as valency permits. In certain embodiments, d is 0. In certain embodiments, d is 1. In certain embodiments, d is 2. In certain embodiments, d is 3, as valency permits. In certain embodiments, d is 4, as valency permits. In certain embodiments, d is 5, as valency permits. In certain embodiments, d is 6, as valency permits.

In certain embodiments, d is 0.

In certain embodiments, q is an integer from 0 to 2. In certain embodiments, q is 0.

In certain embodiments, q is 1. In certain embodiments, q is 2.

In certain embodiments, q is 1.

In certain embodiments, T is Formula I-3-i

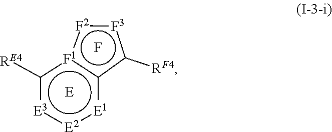

wherein,
E¹ is

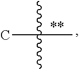

### CR^{E1}, or N;

E² is

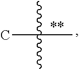

### CR^{E2}, or N;

E³ is

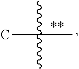

### CR^{E3}, or N;

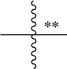

denotes attachment to L;
R^{E1}, R^{E2}, R^{E3}, and R^{E4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
F¹ is C or N;
F² is NR^{F2′} or CR^{F2};
R^{F2′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R^{F2} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —R^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R;
F³ is CR^{F3}, N, or NR^{F3′};
R^{F3} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —R^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R^{F3′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R^{F4} is —C(═O)NR^{3a}R^{3b} or 5- to 6-membered heteroaryl optionally substituted with one or more R; and
R^{3a} and R^{3b} are independently hydrogen, C₁₋₆ alkyl, C₂₋₆ alkenyl, C₂₋₆ alkynyl, C₃₋₁₂ carbocyclyl, 3to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R; or
R^{3a} and R^{3b}, together with the nitrogen atom to which they are attached, form 3- to 12-membered heterocycle or 5- to 10-membered heteroaryl, wherein the heterocycle or heteroaryl is optionally substituted with one or more R.

In certain embodiments, T is of Formula I-3-i-a

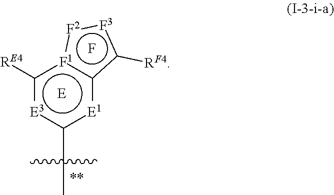

In certain embodiments, T is of Formula I-3-i-b

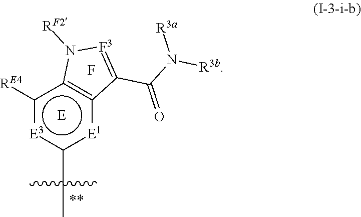

In certain embodiments, E¹ is

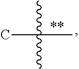

### CR^{E1}, or N.

In certain embodiments, E² is

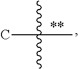

### CR^{E2}, or N.

In certain embodiments, E³ is

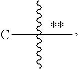

### CR^{E3}, or N.

In certain embodiments, one of E¹, E², and E³ is N. In certain embodiments, two of E¹, E², and E³ are N.

In certain embodiments, R^{E1}, R^{E2}, R^{E3}, and R^{E4} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R.

In certain embodiments, R^{E1}, R^{E2}, R^{E3}, and R^{E4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{E1}, R^{E2}, R^{E3}, and R^{E4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{E1}, R^{E2}, R^{E3}, and R^{E4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{E1}, R^{E2}, R^{E3}, and R^{E4} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, R^{E1} and R^{E3} are independently or R^{E1} or R^{E2} is hydrogen, halogen, or C₁₋₆ alkyl optionally substituted with one or more R.

In certain embodiments, R^{E4} is hydrogen, halogen, —CN, C₁₋₆ alkyl, C₁₋₆ alkoxy, C₁₋₆ alkylamino, C₃₋₁₂ carbocyclyl, 3- to 12-membered heterocyclyl, or —S(═O)₂R^{a}, wherein the alkyl, alkoxy, alkylamino, carbocyclyl, or heterocyclyl is optionally substituted with one or more R^{u}.

In certain embodiments, F¹ is C or N.

In certain embodiments, F² is NR^{F2′} or CR^{F2}.

In certain embodiments, F³ is NR^{F3′}, N, or CR^{F3}

In certain embodiments, R^{F2′} and R^{F3′} are independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{F2′} and R^{F3′} are independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R.

In certain embodiments, R^{F2′} and R^{F3′} are independently hydrogen, C₁₋₆ alkyl, C₂₋₆ alkenyl, C₂₋₆ alkynyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, R^{F2′} and R^{F3′} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R.

In certain embodiments, R^{F2′} is hydrogen or C₁₋₆ alkyl optionally substituted with one or more R.

In certain embodiments, R^{F2} and R^{F3} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —R^{b}C(═O)NR^{c}R^{d}—NR^{b}C(═O)R^{a}, —NR^{b}C(O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R.

In certain embodiments, R^{F2} and R^{F3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{F2} and R^{F3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{F2} and R^{F3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{F2} and R^{F3} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, R^{F3} is hydrogen.

In certain embodiments, R^{F4} is —C(═O)NR^{3a}R^{3b} or 5- to 6-membered heteroaryl (e.g., heteroaryl comprising one 5- or 6-membered ring and 1-3 heteroatoms selected from N, O, and S) optionally substituted with one or more R.

In certain embodiments, R^{3a} and R^{3b} are independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{3a} and R^{3b} are independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R.

In certain embodiments, R^{3a} and R^{3b} are independently hydrogen, C₁₋₆ alkyl, C₂₋₆ alkenyl, C₂₋₆ alkynyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, R^{3a} and R^{3b} are independently hydrogen, C₁₋₆ alkyl, C₃₋₆ carbocyclyl, 3- to 6-membered heterocyclyl, —S(═O)₂R^{a}, —S(═O)₂0R^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R.

In certain embodiments, R^{3a} and R^{3b}, together with the nitrogen atom to which they are attached, form 3- to 12-membered heterocycle (e.g., heterocyclyl comprising one or two 3- to 8-membered rings and 1-5 heteroatoms selected from N, O, and S) or 5- to 10-membered heteroaryl (e.g., heteroaryl comprising one or two 5- or 6-membered rings and 1-5 heteroatoms selected from N, O, and S), wherein the heterocycle or heteroaryl is optionally substituted with one or more R.

In certain embodiments, R^{3a} and R^{3b} are independently hydrogen or C₁₋₆ alkyl optionally substituted with one or more R.

In certain embodiments, T is of Formula I-3-ii

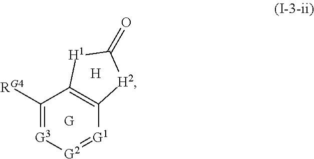

wherein:
G¹ is

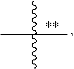

### N, or CR^{G1};

G² is

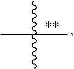

### N or CR^{G2};

G³ is

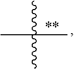

### N, or CR^{G3};

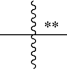

denotes attachment to L;
R^{G1}, R^{G2}, R^{G3}, and R^{G4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
H¹ is C(R^{H1})₂, NR^{H1′} or O;
H² is C(R^{H2})₂, NR^{H2′} or O;
each occurrence of R^{H11} and R^{H2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl wherein the alkyl, alkoxy, alkylamino, alkenyl, alkynyl, carbocyclyl, heterocyclyl, aryl, or heteroaryl is optionally substituted with one or more R^{u}; or
two R^{H1} or two R^{H2}, together with the carbon atom to which they are attached, form C₃₋₆ carbocycle or 3- to 6-membered heterocycle, wherein the carbocycle or heterocycle is optionally substituted with one or more R^{u}; and
R^{H1′} and R^{H2′} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R.

In certain embodiments, T is of Formula I-3-ii-a

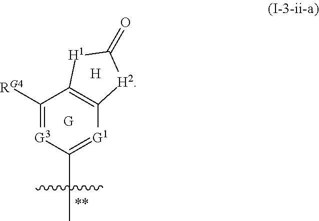

In certain embodiments, G¹ is

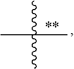

### N, or CR^{G1}.

In certain embodiments G² is

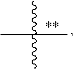

### N, or CR^{G2}.

In certain embodiments, G³ is

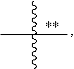

### N, or CR^{G3}.

In certain embodiments, R^{G1}, R^{G2}, R^{G3}, and R^{G4} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —R^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R.

In certain embodiments, R^{G}i, R^{G2}, R^{G3}, and R^{G4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{G}i, R^{G2}, R^{G3}, and R^{G4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{G}i, R^{G2}, R^{G3}, and R^{G4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{G1}, R^{G2}, R^{G3}, and R^{G4} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, R^{G1} and R^{G3} are both hydrogen.

In certain embodiments, R^{G4} is hydrogen, C₁₋₆ alkyl, C₁₋₆ alkoxy, C₁₋₆ alkylamino, C₂₋₆ alkenyl, or 3- to 12-membered heterocyclyl, wherein the alkyl, alkoxy, alkylamino, alkenyl, or heterocyclyl is optionally substituted with one or more R.

In certain embodiments, H¹ is C(R^{H}i)₂, NR^{H11′} or O.

In certain embodiments, H² is C(R^{H2})₂, NR^{H2′} or O.

In certain embodiments, H¹ is NR^{H11′} and H² is NR^{H2}′

In certain embodiments, each occurrence of R^{H1} and R^{H2} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R.

In certain embodiments, each occurrence of R^{H1} and R^{H2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R^{H1} and R^{H2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each occurrence of R^{H1} and R^{H2} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, R^{H1′} and R^{H2′} are independently C₁₋₆ alkyl

In certain embodiments, two R^{H}i or two R^{H2}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S), wherein the carbocycle or heterocycle is optionally substituted with one or more R.

In certain embodiments, R^{H11′} and R^{H2′} are independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, T is of Formula I-3-iii

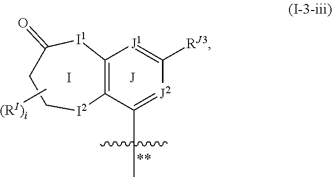

wherein:

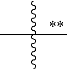

denotes attachment to L;
I¹ is C(Rn)₂, NR^{I1′}, or O;
I² is C(R^{I2})₂, NR^{I2′}, or O;
each occurrence of R¹ and R^{I2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R¹ or two R^{I2}, together with the carbon atom to which they are attached, form C₃₋₆ carbocycle or 3- to 6-membered heterocycle, wherein the carbocycle or heterocycle is optionally substituted with one or more R^{u};
R^{I1′} and R^{I2′} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}
each R¹ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
i is an integer selected from 0 to 4;
J¹ is N or CR^{J1};
J² is N or CR^{J2}; and
R^{J1}, R^{J2}, and R^{J3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, T is of Formula I-3-iii-a

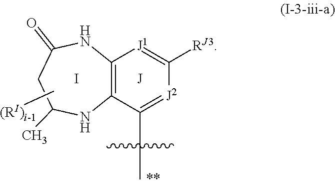

In certain embodiments, I¹ is C(R¹)₂, NR^{I1′}, or O.

In certain embodiments, I² is C(R^{I2})₂, NR^{I2′}, or O.

In certain embodiments, I¹ is NR^{I1′} and I² is NR^{I2′};

In certain embodiments, each occurrence of R¹ and R^{I2} are independently oxo, hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R″ and R^{I2} are independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R″ and R^{I2} are independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each occurrence of R″ and R^{I2} are independently oxo, hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, two R″ or two R^{I2}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3to 6-membered ring and 1-3 heteroatoms selected from N, O, and S), wherein the carbocycle or heterocycle is optionally substituted with one or more R;

In certain embodiments, R^{I1} and R^{I2} are independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, R^{I1′} and R^{I2′} are independently hydrogen or C₁₋₆ alkyl.

In certain embodiments, each R¹ is independently oxo, hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —R^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R.

In certain embodiments, each Ri is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, each R¹ is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, each R¹ is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R¹ is C₁₋₆ alkyl. In certain embodiments, at least one R¹ is C₁₋₆ alkyl.

In certain embodiments, each R¹ is independently oxo, hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, i is an integer selected from 0 to 4. In certain embodiments, i is 0. In certain embodiments, i is 1. In certain embodiments, i is 2. In certain embodiments, i is 3. In certain embodiments, i is 4.

In certain embodiments, J¹ is N or CR.

In certain embodiments, J² is N or CR².

In certain embodiments, R¹, R^{J2}, and R³ are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —R^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R.

In certain embodiments, R^{J1}, R^{J2}, and R³ are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{J1}, R^{J2}, and R³ are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{J1}, R^{J2}, and R^{J3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{J1}, R^{J2}, and R^{J3} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, R^{J3} is hydrogen, halogen, C₁₋₆ alkyl, 3- to 12-membered heterocyclyl, 5- to 10-membered heteroaryl, or —C(═O)NR^{c}R^{d}, wherein the alkyl, heterocyclyl, or heteroaryl is optionally substituted with one or more R.

In certain embodiments, R^{J1} and R^{J2} are independently hydrogen or C₁₋₆ alkyl optionally substituted with one or more R.

In certain embodiments, T is of Formula I-3-iv

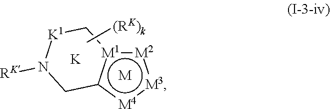

wherein:
M¹ is N or C;
M² is

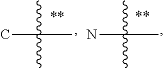

### N, or CR^{M2};

M³ is

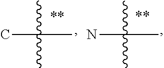

### or CR^{M3};

M⁴ is N

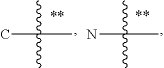

### N, or CR^{M4}

wherein

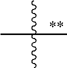

denotes attachment to L; and at least one of M¹, M², M³, and M⁴ is N or

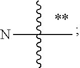 [SMILES: *N]

R^{M2}, R^{M3}, and or R^{M4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R^{K′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
K¹ is —C(═O)— or —C(R^{K1})₂—;
each R^{K1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}
each R^{K} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
k is an integer selected from 0 to 4.

In certain embodiments, T is of Formula I-3-iv-a

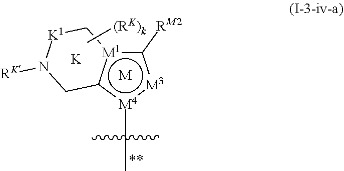

In certain embodiments, T is of Formula I-3-iv-b

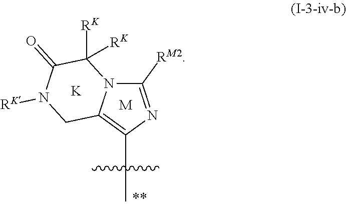

In certain embodiments M¹ is N or C.

In certain embodiments, M² is

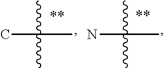

### N, or CR^{M2}.

In certain embodiments, M³ is

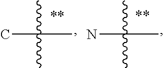

### N, or CR^{M3}

In certain embodiments, M³ is N.

In certain embodiments, M⁴ is

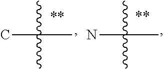

### N, or CR^{M4}.

In certain embodiments, at least one of M¹, M², M³, and M⁴ is N or

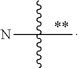 [SMILES: *N]

In certain embodiments, at least two of M¹, M³, and M⁴ are N. In certain embodiments, at least one of M¹, M³, and M⁴ is N.

In certain embodiments, R^{M2}, R^{M3}, and or R^{M4} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —R^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{M2}, R^{M3}, and or R^{M4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{M2}, R^{M3}, and or R^{M4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{M2}, R^{M3}, and or R^{M4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{M2}, R^{M3}, and or R^{M4} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, R^{M2} is C₁₋₆ alkyl, C₁₋₆ alkoxy, or 3- to 12-membered heterocyclyl, wherein the alkyl, alkoxy, or heterocyclyl is optionally substituted with one or more R.

In certain embodiments, R^{M3} is hydrogen.

In certain embodiments, R^{K′} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{K′} is hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{K′} is hydrogen, C₁₋₆ alkyl, C₂₋₆ alkenyl, C₂₋₆ alkynyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, R^{K′} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, R^{K′} is hydrogen or C₁₋₆ alkyl optionally substituted with one or more R.

In certain embodiments, K¹ is —C(═O)— or —C(R^{K1})₂—.

In certain embodiments, each R^{K1} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{K1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, each R^{K1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R.

In certain embodiments, each R^{K1} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, each R^{K} is independently oxo, hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —R^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R.

In certain embodiments, each R^{K} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, each R^{K} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, each R^{K} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{K} is independently oxo, hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, each R^{K} is independently hydrogen or C₁₋₆ alkyl. In certain embodiments, at least one R^{K} is hydrogen. In certain embodiments, at least one R^{K} is C₁₋₆ alkyl.

In certain embodiments, one R^{K} is hydrogen, the other geminal R^{K} is C₁₋₆ alkyl.

In certain embodiments, k is an integer selected from 0 to 4. In certain embodiments, k is 0. In certain embodiments, k is 1. In certain embodiments, k is 2. In certain embodiments, k is 3. In certain embodiments, k is 4.

In certain embodiments, T is of Formula I-3-v

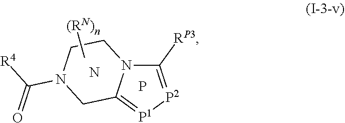

wherein:
P¹ is

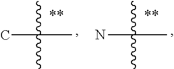

### N or CR^{P1}

P² is

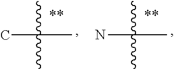

### N, or CR^{P2}.

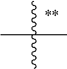

denotes attachment to L;
each R^{N} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
n is an integer selected from 0 to 6;
R^{P1}, R^{P2}, and R^{P3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
R⁴ is —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, T is of Formula I-3-v-a

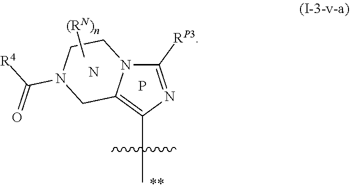

In certain embodiments, P¹ is

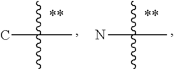

### N, or CR^{P1}.

In certain embodiments, P² is

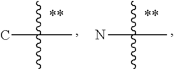

### N, or CR^{P2}.

In certain embodiments, each R^{N} is independently oxo, hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—NR^{b}C(═O)R^{a}, —NR^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R.

In certain embodiments, each R^{N} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, each R^{N} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, each R^{N} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{N} is independently oxo, hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, n is an integer selected from 0 to 6. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6.

In certain embodiments, n is 0.

In certain embodiments, R^{P1}, R^{P2}, and R^{P3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{P1}, R^{P2}, and R^{P3} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R.

In certain embodiments, R^{P1}, R^{P2}, and R^{P3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{P1}, R^{P2}, and R^{P3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{P1}, R^{P2}, and R^{P3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{P1}, R^{P2}, and R^{P3} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, R^{P3} is hydrogen, C₁₋₆ alkyl, C₃₋₁₂ carbocyclyl, 3- to 12-membered heterocyclyl, or —C(═O)NR^{c}R^{d}, wherein the alkyl, carbocyclyl, or heterocyclyl is optionally substituted with one or more R.

In certain embodiments, R⁴ is —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, R⁴ is C₁₋₆ alkyl or C₁₋₆ alkylamino, wherein the alkyl or alkylamino is optionally substituted with one or more R^{u}.

In certain embodiments, T is of Formula I-3-vi

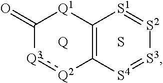

wherein:
Q¹ is NR^{Q1′}, C(R^{Q1})₂, or O;
R^{Q1′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
each R^{Q1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{Q1}, together with the carbon atom to which they are attached, form C₃₋₆ carbocycle or 3- to 6-membered heterocycle, wherein the carbocycle or heterocycle is optionally substituted with one or more R^{u};
when [symbol] is a double bond, then Q² is N or CR^{Q2}; and Q³ is N or CR^{Q3};
when [symbol] is a single bond, then Q² is NR^{Q2′}, C(R^{Q2})₂, or O; Q³ is NR^{Q3′}, C(R^{Q3})₂, or O;
R^{Q2′} and R^{Q3′} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
each occurrence of R^{Q2} and R^{Q3} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{Q2} or two R^{Q3}, together with the carbon atom to which they are attached, form C₃₋₆ carbocycle or 3- to 6-membered heterocycle, wherein the carbocycle or heterocycle is optionally substituted with one or more R;
S¹ is CR^{S1} or N;
S² is

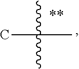

### CR^{S2}, or N;

S³ is

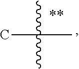

### CR^{S3}, or N;

S⁴ is

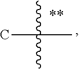

### CR^{S4}, or N;

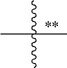

denotes attachment to L; and
R^{S1}, R^{S2}, R^{S3}, and R^{S4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂₀R^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, T is of Formula I-3-vi-a or I-3-vi-b

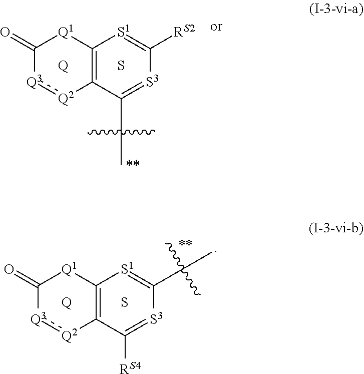

In certain embodiments, Q¹ is NR^{1′}, C(R^{Q1})₂, or O.

In certain embodiments, R^{Q1′} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂0R^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{Q1′} is hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{Q1′} is hydrogen, C₁₋₆ alkyl, C₂₋₆ alkenyl, C₂₋₆ alkynyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, R^{Q1′} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, R^{Q1′} is hydrogen or C₁₋₆ alkyl.

In certain embodiments, each R^{Q1} is independently oxo, hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{Q1} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, each R^{Q1} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{Q1} is independently oxo, hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, two R^{Q1}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered rings and 1-3 heteroatoms selected from N, O, and S), wherein the carbocycle or heterocycle is optionally substituted with one or more R.

In certain embodiments, when is a double bond, then Q² is N or CR^{Q2}.

In certain embodiments, when is a single bond, then Q² is NR^{Q2′}, C(R^{Q2})₂, or O.

In certain embodiments, R^{Q2′} and R^{Q3′} are independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{Q2′} and R^{Q3′} are independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R.

In certain embodiments, R^{Q2′} and R^{Q3′} are independently hydrogen, C₁₋₆ alkyl, C₂₋₆ alkenyl, C₂₋₆ alkynyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, R^{Q2′} and R^{Q3′} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R.

In certain embodiments, each occurrence of R^{Q2} and R^{Q3} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R.

In certain embodiments, each occurrence of R^{Q2} and R^{Q3} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R^{Q2} and R^{Q3} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each occurrence of R^{Q2} and R^{Q3} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, each R^{Q2} is hydrogen.

In certain embodiments, two R^{Q2} or two R^{Q3}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered rings and 1-3 heteroatoms selected from N, O, and S), wherein the carbocycle or heterocycle is optionally substituted with one or more R^{u}.

In certain embodiments, S¹ is CR^{S1} or N.

In certain embodiments, S² is

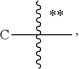

### CR^{S2}, or N.

In certain embodiments, S³ is

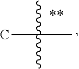

### CR^{S3}, or N.

In certain embodiments, S⁴ is

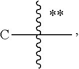

### CR^{S4}, or N.

In certain embodiments, R^{S1}, R^{S2}, R^{S3}, and R^{S4} are independently oxo, hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R.

In certain embodiments, R^{S1}, R^{S2}, R^{S3}, and R^{s4} are independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{S1}, R^{S2}, R^{S3}, and R^{S4} are independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{S1}, R^{S2}, R^{S3}, and R^{S4} are independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{S1}, R^{S2}, R^{S3}, and R^{S4} are independently oxo, hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, Rsi and R^{S3} are both hydrogen.

In certain embodiments, R^{S2} is hydrogen, C₁₋₆ alkyl, C₁₋₆ alkoxy, or 3-12-membered heterocyclyl.

In certain embodiments, R^{S4} is hydrogen or C₁₋₆ alkyl.

In certain embodiments, L is of Formula I-2

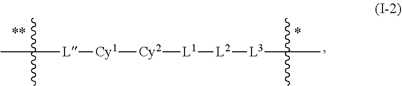

wherein.
* denotes attachment to T and * denotes attachment to C;
L″ is absent; or
L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u};
R^{L} is hydrogen, C₁₋₆ alkyl, C₁₋₆ haloalkyl, C₃₋₆ carbocyclyl, or 3- to 6-membered heterocyclyl, wherein the alkyl, haloalkyl, carbocyclyl, or heterocyclyl is optionally substituted with one or more R^{u};
wherein:
i) when L″ is C₁₋₃ alkylene, —C(═O)—, —C(═O)N(R^{L})—, —C(═O)O—, —N(R^{L})—, —O—, —S—, or —S(═O)₂, wherein the alkylene is optionally substituted with one or more R^{u}, then Cy¹ is optionally substituted C₆ arylene or 5- to 6-membered heteroarylene;
ii) when L″ is absent, then
   Cy¹ is

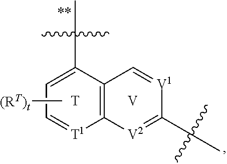

wherein:
** denotes attachment to T;
T¹ is CR^{T1} or N;
V¹ is CRy¹ or N;
V² is CRV² or N;
R^{T1}, R^{V1}, and RV² are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R;
each R^{T} is independently halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
t is an integer selected from 0 to 6, as valency permits,
wherein R^{T} may be present on either Ring T or Ring V;
or
Cy¹ is

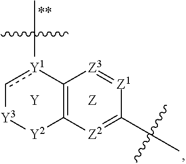

wherein:
when [symbol] is single bond, Y¹ is N or CR^{Y1}; when [symbol] is double bond, Y¹ is C;
* denotes attachment to T;
R^{Y1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
Y² is C(R^{Y2})₂, O, or N(R^{Y2′})
each R^{Y2} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; or
two R^{Y2} together form an oxo;
R^{Y2′} is hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
Y³ is C(R^{Y3})₂ or N(R);
each R^{Y3} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
R^{Y2′} is hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
Z¹ is CR^{Z1} or N;
Z² is CR^{Z2} or N;
Z³ is CR^{Z3} or N;
R^{Z1}, R^{Z2}, R^{Z3} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R;
Cy² is 5- to 6-membered heteroarylene optionally substituted with one or more R^{u};
L¹ is —C(═O)NH— or 5- to 6-membered heteroaryl optionally substituted with one or more R^{u};
L² is —C(R^{L2})₂—, wherein
each R^{L2} is independently hydrogen, deuterium, C₁₋₃ alkyl, or C₁₋₃ haloalkyl; or two geminal R^{L2}, together with the carbon atom to which they are bonded, form C₃₋₅ carbocyclyl or 3- to 5-membered heterocyclyl, wherein the carbocyclyl or heterocyclyl is optionally substituted with one or more R^{u};
and
L³ is E-ethenylene, ethynylene, C₆ arylene, or 5- to 6-membered heteroarylene, wherein the E-ethenylene, arylene, or heteroarylene is optionally substituted with one or more R^{u}.

In certain embodiments, L is of Formula I-2

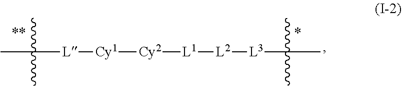

wherein:
* denotes attachment to T and * denotes attachment to C;
L″ is absent; or
L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u};
R^{L} is hydrogen, C₁₋₆ alkyl, C₁₋₆ haloalkyl, C₃₋₆ carbocyclyl, or 3- to 6-membered heterocyclyl, wherein the alkyl, haloalkyl, carbocyclyl, or heterocyclyl is optionally substituted with one or more R^{u};
wherein:
i) when L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u}, then Cy¹ is optionally substituted C₆ arylene or 5- to 6-membered heteroarylene;
ii) when L″ is absent, then
   Cy¹ is

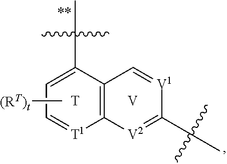

wherein:
** denotes attachment to T;
T¹ is CR^{T1} or N;
V¹ is CR^{V1} or N;
V² is CR^{V2} or N;
R^{T1}, R^{V1}, and R^{V2} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
each R^{T} is independently halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
t is an integer selected from 0 to 6, as valency permits;
wherein R^{T} may be present on either Ring T or Ring V;
or
Cy¹ is

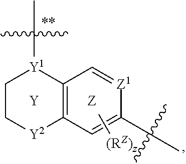

wherein:
Y¹ is N or CR^{Y1};
** denotes attachment to T;
R^{Y1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
Y² is C(R^{Y2})₂, O, or N(R^{Y2′}) each R^{Y2} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
R^{Y2′} is hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
Z¹ is CR^{Z1} or N;
R^{Z1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, 3- to 4-membered heterocyclyl, wherein the alkyl, alkoxy, alkylamino, carbocyclyl, or heterocyclyl is optionally substituted with one or more R^{u};
each R^{Z} is independently oxo, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
z is an integer selected from 0 to 6, as valency permits,
wherein R^{Z} may be present on either Ring Y or Ring Z;
Cy² is 5- to 6-membered heteroarylene optionally substituted with one or more R^{u};
L¹ is —C(═O)NH— or 5- to 6-membered heteroaryl optionally substituted with one or more R^{u};
L² is C(R^{L2})₂, wherein
each R^{L2} is independently hydrogen, C₁₋₃ alkyl, or C₁₋₃ haloalkyl; or
two geminal R^{L2}, together with the carbon atom to which they are bonded, form C₃₋₅ carbocyclyl or 3- to 5-membered heterocyclyl, wherein the carbocyclyl or heterocyclyl is optionally substituted with one or more R^{u}; and
L³ is E-ethenylene, ethynylene, C₆ arylene, or 5- to 6-membered heteroarylene, wherein the E-ethenylene, arylene, or heteroarylene is optionally substituted with one or more R^{u}.

In certain embodiments, L″ is absent.

In certain embodiments, L″ is C₁₋₃ alkylene (e.g., methylene, ethylene, n-propylene, or iso-propylene), —C(═O)—, —C(═O)N(R^{L})—, —C(═O)O—, —N(R^{L})—, —O—, —S—, or —S(═O)₂—, wherein the alkylene is optionally substituted with one or more R^{u}.

In certain embodiments, Cy¹ is optionally substituted C₆ arylene (i.e., phenylene, e.g., 1,2-phenylene, 1,3-phenylene, or 1,4-phenylene) or 5- to 6-membered heteroarylene (e.g., heteroarylene comprising one 5- to 6-membered ring and 1-4 heteroatoms selected from O, N, and S).

In certain embodiments, when L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u}, then Cy¹ is optionally substituted C₆ arylene or 5- to 6-membered heteroarylene.

In certain embodiments, R^{L} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ haloalkyl (e.g., C₁₋₆ alkyl substituted with one or more halogen (e.g., —F, —Cl, —Br, or —I)), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, haloalkyl, carbocyclyl, or heterocyclyl is optionally substituted with one or more R.

In certain embodiments, Cy¹ is optionally substituted with one or more substitutent selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —N^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, and —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, Cy¹ is optionally substituted with one or more R^{u}. In certain embodiments, Cy¹ is optionally substituted with one or more R^{V′1}. In certain embodiments, Cy¹ is optionally substituted with one or more R^{V′}.

In certain embodiments, R^{u}; is R^{V′1}. In certain embodiments, R^{u}; is R^{V′}.

In certain embodimegits, Cy¹ is

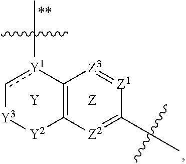

wherein:
when [symbol] is single bond, Y¹ is N or CR^{Y1}; when [symbol] is double bond, Y¹ is C;
** denotes attachment to T;
R^{Y1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
Y² is C(R^{Y2})₂, O, or N(R^{Y2′})
each R^{Y2} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; or
two R^{Y2} together form an oxo;
R^{Y2′} is hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
Y³ is C(R^{Y3})₂ or N(R);
each R^{Y3} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
R^{Y2′} is hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
Z¹ is CR^{Z1} or N;
Z² is CR^{Z2} or N;
Z³ is CR^{Z3} or N;
R^{Z1}, R^{Z2}, R^{Z3} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};

In certain embodiments, Cy¹ is

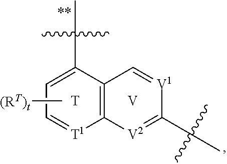

In certain embodiments, when L″ is absent, then Cy¹ is

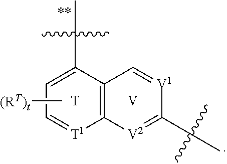

In certain embodiments, Cy¹ is

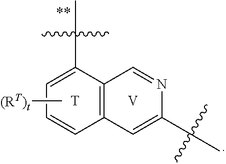

In certain embodiments, when L″ is absent, then Cy¹ is

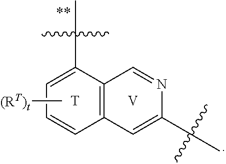

In certain embodiments, T¹ is CR^{T}i or N. In certain embodiments, T¹ is CR^{T}l. In certain embodiments, T¹ is N.

In certain embodiments, V¹ is CRy¹ or N. In certain embodiments, V¹ is CR^{V1}. In certain embodiments, V² is N.

In certain embodiments, V² is CRV² or N. In certain embodiments, V² is CRV². In certain embodiments, V² is N.

In certain embodiments, R^{T1}, R^{V1}, and R^{V2} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R.

In certain embodiments, R^{T1}, R^{V1}, and R^{V2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R.

In certain embodiments, R^{T1}, R^{V}l, and R^{V2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{T1}, R^{V1}, and R^{V2} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R.

In certain embodiments, R^{T}i and R^{V2} are both hydrogen.

In certain embodiments, each R^{T} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R.

In certain embodiments, each RT is independently C₁₋₆ alkyl. In certain embodiments, at least one RT is C₁₋₆ alkyl.

In certain embodiments, t is 0. In certain embodiments, t is 1. In certain embodiments, t is 2. In certain embodiments, t is 3. In certain embodiments, t is 4. In certain embodiments, t is 5. In certain embodiments, t is 6.

In certain embodiments, Cy¹ is

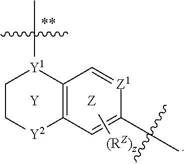

In certain embodiments, Cy¹ is

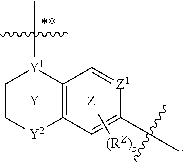

In certain embodiments, when L″ is absent, then Cy¹ is

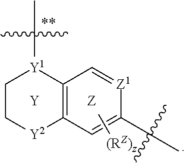

In certain embodiments, Cy¹ is

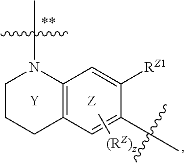

wherein R^{Z1} is C₁₋₆ alkyl substituted with one or more halogen.

In certain embodiments, when L″ is absent, then Cy¹ is

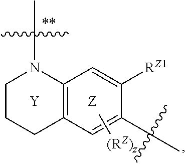

wherein R^{Z1} is C₁₋₆ alkyl substituted with one or more halogen.

In certain embodiments, Cy¹ is

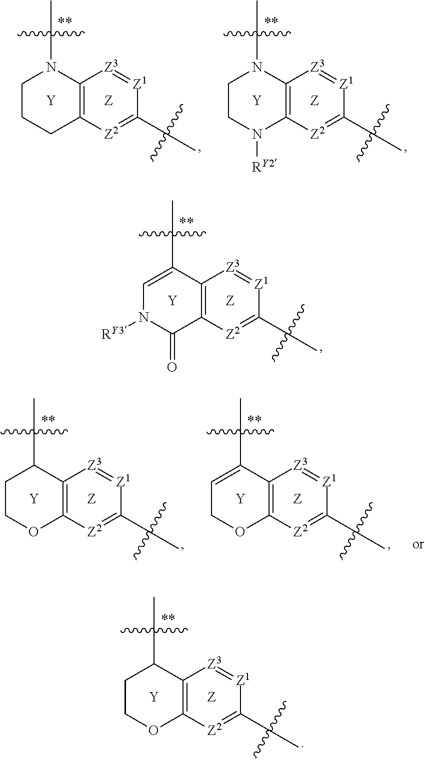

In certain embodiments, Y¹ is N or CR^{Y1}. In certain embodiments, Y¹ is N. In certain embodiments, Y¹ is CR^{Y1}.

In certain embodiments, when [symbol] is single bond, Y¹ is N or CR^{Y1}; when [symbol] is double bond, Y¹ is C.

In certain embodiments, R^{Y1} is hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R^{u}.

In certain embodiments, Y² is C(R^{Y2})₂, O, or N(R^{Y2′}))

In certain embodiments, each R^{Y2} is independently hydrogen, halogen (e.g., —F, —C₁, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R.

In certain embodiments, each R^{I2} is hydrogen.

In certain embodiments, two R^{Y2} together form an oxo.

In certain embodiments, R^{Y2′} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₄ carbocyclyl (e.g., cyclopropyl (C₃), cyclopropenyl (C₃), cyclobutyl (C₄), or cyclobutenyl (C₄)), 3- to 4-membered heterocyclyl (e.g., heterocyclyl comprising one 3- to 4-membered rings and 1 heteroatom selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, Y³ is C(R^{Y3})₂ or N(R^{Y}3′).

In certain embodiments, each R^{Y3} is independently hydrogen, halogen (e.g., —F, —C₁, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R^{u}.

In certain embodiments, R^{Y3′} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₄ carbocyclyl (e.g., cyclopropyl (C₃), cyclopropenyl (C₃), cyclobutyl (C₄), or cyclobutenyl (C₄)), 3- to 4-membered heterocyclyl (e.g., heterocyclyl comprising one 3- to 4-membered rings and 1 heteroatom selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, Z¹ is CR^{Z1} or N. In certain embodiments, Z² is CR^{Z2} or N.

In certain embodiments, Z³ is CR^{Z3} or N.

In certain embodiments, Z¹ is CR^{Z1} and Z² is CR^{Z2}.

In certain embodiments, one of Z¹ and Z² is N or each of Z¹ and Z² is N.

In certain embodiments, Z³ is CH or N.

In certain embodiments, R^{Z1}, R^{Z2}, and R^{Z3} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one 3- to 4-membered rings and 1 heteroatom selected from N, O, and S), wherein the alkyl, alkoxy, alkylamino, carbocyclyl, or heterocyclyl is optionally substituted with one or more R.

In certain embodiments, R^{Z1} or R^{Z2}, when available, is hydrogen, halogen, —CN, C₁₋₆ alkyl, or C₁₋₆ haloalkyl.

In certain embodiments, each R^{Z} is independently oxo, hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one or two 3- to 4-membered rings and 1 heteroatom selected from N, O, and S), wherein the alkyl, alkoxy, alkylamino, carbocyclyl, or heterocyclyl is optionally substituted with one or more R.

In certain embodiments, at least one R^{Z} is oxo.

In certain embodiments, z is 0. In certain embodiments, z is 1. In certain embodiments, z is 2, as valency permits. In certain embodiments, z is 3, as valency permits. In certain embodiments, z is 4, as valency permits. In certain embodiments, z is 5, as valency permits. In certain embodiments, z is 6, as valency permits.

In certain embodiments, Cy¹ is

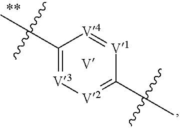

wherein.
** denotes attachment to L″;
V′¹ is CR^{V′1} or N;
V′² is CR^{V′2} or N;
V′³ is CR^{V′3} or N;
V′⁴ is CR^{V′4} or N;
R^{V′1}, R^{V′2}, R^{V′3}, and R^{V′4} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}.

In certain embodiments, when L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u}, then Cy¹ is

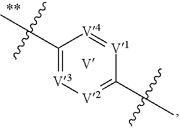

wherein.
** denotes attachment to L″;
V′¹ is CR^{V′1} or N;
V′² is CR^{V′2} or N;
V′³ is CR^{V′3} or N;
V′⁴ is CR^{V′4} or N;
R^{V′1}, R^{V′2}, R^{V′3}, and R^{V′4} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}.

In certain embodiments, Cy¹ is

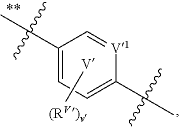

wherein.
** denotes attachment to L″;
V^{′1} is CR^{V′1} or N;
R^{V′1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R;
each R^{V′} is independently halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; and
v′ is an integer selected from 0 to 5.

In certain embodiments, when L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u}, then Cy¹ is

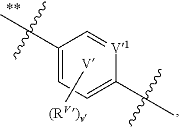

wherein.
** denotes attachment to L″;
V′¹ is CR^{V′1} or N;
R^{V′1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
each R^{V′} is independently halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; and
v′ is an integer selected from 0 to 5.

In certain embodiments, V^{′1} is CR^{V′1} or N. In certain embodiments, V^{′1} is CR^{V′1}.

In certain embodiments, V′¹ is N.

In certain embodiments, V′² is CR^{V′2} or N. In certain embodiments, V′² is CR^{V′2}.

In certain embodiments, V′³ is N.

In certain embodiments, V′³ is CR^{V′3} or N. In certain embodiments, V′³ is CR^{V′3}.

In certain embodiments, V′³ is N.

In certain embodiments, V′³ is CR^{V′3} or N. In certain embodiments, V′³ is CR^{V′3}.

In certain embodiments, V′³ is N.

In certain embodiments, each of V′¹ and V′² is N; or V′¹ is N, and V′² is CR^{V′2}, wherein R^{V′2} is hydrogen, halogen, C₁₋₆ alkyl, or C₁₋₆ haloalkyl.

In certain embodiments, one of V′³ and V′⁴ is N; or V′³ is CR^{V′3} and V′⁴ is CR^{V′4}, wherein R^{V′3} and/or R^{V′4}, when available, are independently hydrogen, halogen, —CN, C₁₋₆ alkyl, C₁₋₆ haloalkyl, C₁₋₆ alkoxy, or C₁₋₆ alkylamino.

In certain embodiments, each R^{V′1}, R^{V′2}, R^{V′3}, and R^{V′4} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one or two 3- to 4-membered rings and 1 heteroatom selected from N, O, and S), wherein the alkyl, alkoxy, alkylamino, carbocyclyl, or heterocyclyl is optionally substituted with one or more R.

In certain embodiments, each Rv′ is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₄ carbocyclyl (e.g., cyclopropyl (C₃), cyclopropenyl (C₃), cyclobutyl (C₄), or cyclobutenyl (C₄)), or 3- to 4-membered heterocyclyl (e.g., heterocyclyl comprising one or two 3- to 4-membered rings and 1 heteroatom selected from N, O, and S), wherein the alkyl, alkoxy, alkylamino, carbocyclyl, or heterocyclyl is optionally substituted with one or more R^{u}.

In certain embodiments, v′ is an integer selected from 0 to 5. In certain embodiments, v′ is 0. In certain embodiments, v′ is 1. In certain embodiments, v′ is 2. In certain embodiments, v′ is 3. In certain embodiments, v′ is 4. In certain embodiments, v′ is 5.

In certain embodiments, Cy² is 5- to 6-membered heteroaryl (e.g., heteroaryl comprising one 5- or 6-membered rings and 1-4 heteroatoms selected from N, O, and S), wherein heteroarylene is optionally substituted with one or more R.

In certain embodiments, Cy² is pyridinylene optionally substituted with one or more halogen, C₁₋₆ alkyl, or C₁₋₆ alkoxy.

In certain embodiments, Cy² is

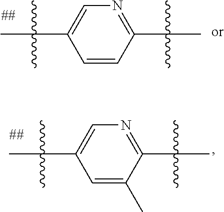

wherein ##denotes attachment to Cy¹.

In certain embodiments, L¹ is —C(═O)NH— or 5- to 6-membered heteroaryl optionally substituted with one or more R.

In certain embodiments, L¹ is —C(═O)NH—.

In certain embodiments, L¹ is —C(═O)NH—, wherein #denotes attachment to Cy².

In certain embodiments, L¹ is 5- to 6-membered heteroaryl (e.g., heteroaryl comprising one 5- or 6-membered ring and 1-4 heteroatoms selected from O, N, or S) optionally substituted with one or more R.

In certain embodiments, L² is C(R^{L2})₂, wherein each R^{L2} is independently hydrogen, deuterium, C₁₋₃ alkyl (e.g., methyl (C₁), ethyl (C₂), n-propyl (C₃), or i-propyl (C₃)), or C₁₋₃ haloalkyl (e.g., methyl (C₁), ethyl (C₂), n-propyl (C₃), or i-propyl (C₃) substituted with one or more halogen (e.g., —F, —Cl, —Br, or —I)).

In certain embodiments, L² is C(R^{L2})₂, wherein each R^{L2} is independently hydrogen, C₁₋₃ alkyl (e.g., methyl (C₁), ethyl (C₂), n-propyl (C₃), or i-propyl (C₃)), or C₁₋₃ haloalkyl (e.g., methyl (C₁), ethyl (C₂), n-propyl (C₃), or i-propyl (C₃) substituted with one or more halogen (e.g., —F, —Cl, —Br, or —I)).

In certain embodiments, two geminal R^{L2}, together with the carbon atom to which they are bonded, C₃₋₅ carbocyclyl (e.g., cyclopropyl (C₃), cyclopropenyl (C₃), cyclobutyl (C₄), cyclobutenyl (C₄), cyclopentyl (C₅), or cyclopentenyl (C₅)) or 3- to 5-membered heterocyclyl (e.g., heterocyclyl comprising one or two 3- to 5-membered rings and 1-3 heteroatoms selected from N, O, and S), wherein the carbocyclyl or heterocyclyl is optionally substituted with one or more R^{u}.

In certain embodiments, L² is CH2.

In certain embodiments, L³ is E-ethenylene, ethynylene, C₆ arylene, or 5- to 6-membered heteroarylene (e.g., heteroaryl comprising one 5- or 6-membered ring and 1-3 heteroatoms selected from N, O, and S), wherein the E-ethenylene, arylene, or heteroarylene is optionally substituted with one or more R.

In certain embodiments, L³ is E-ethenylene optionally substituted with one or more R.

In certain embodiments, L³ is unsubstituted E-ethenylene.

In certain embodiments, L³ is C₆ arylene optionally substituted with one or more R.

In certain embodiments, L³ is -(1,4-phenylene)- optionally substituted with one or more R^{u}.

In certain embodiments, L³ is unsubstituted -(1,4-phenylene)-.

In certain embodiments, L³ is ethynylene.

In certain embodiments, L³ is 5- to 6-membered heteroarylene optionally substituted with one or more R.

In certain embodiments, L³ is unsubstituted 5- to 6-membered heteroarylene.

In certain embodiments, L³ is unsubstituted 5-membered heteroarylene.

In certain embodiments, L³ is unsubstituted 1,3-linked 5-membered heteroarylene.

In certain embodiments, L³ is unsubstituted 6-membered heteroarylene.

In certain embodiments, L³ is unsubstituted 1,4-linked 6-membered heteroarylene.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one or two 3- to 6-membered rings and 1-4 heteroatoms selected from N, O, and S), wherein the alkyl, carbocyclyl, or heterocyclyl is optionally substituted with one or more R.

In certain embodiments, each R^{b} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one or two 3- to 6-membered rings and 1-4 heteroatoms selected from N, O, and S), wherein the alkyl, carbocyclyl, or heterocyclyl is optionally substituted with one or more R.

In certain embodiments, each R^{c} and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one or two 3- to 6-membered rings and 1-4 heteroatoms selected from N, O, and S), wherein the alkyl, carbocyclyl, or heterocyclyl is optionally substituted with one or more R.

In certain embodiments, R^{a}, R^{b}, R^{c}, and R^{d} is independently and optionally substituted with one or more R^{Z}.

In certain embodiments, R^{Z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl.

In certain embodiments, each R^{u}; is independently oxo, deuterium, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NRR^{d}, —NRS(═O)₂R^{a}, —NRS(═O)R^{a}, —N S(═O)₂OR^{b}, —NR^{c}S(═O)₂NR^{c}R^{d}—NR^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u}; is independently oxo, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u}; is independently oxo, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u}; is independently oxo, deuterium, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u}; is independently oxo, deuterium, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, the compound is of Formula I-A

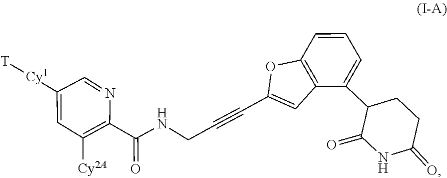

wherein:
Cy^{2A} is hydrogen or C₁₋₆ alkyl;
T is of Formula I-3-ii′ or I-3-vi; and
Formula I-3-i′, Formula I-3-vi, and Cy¹ are as defined herein.

In certain embodiments, Cy^{2A} is hydrogen.

In certain embodiments, Cy^{2A} is C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)).

In certain embodiments, Cy^{2A} is methyl.

In certain embodiments, T of Formula (I-A) is of Formula I-3-ii. In certain embodiments, T of Formula (I-A) is of Formula I-3-ii-a.

In certain embodiments, T of Formula (I-A) is of Formula I-3-ii-a, wherein R^{G4} is hydrogen; G¹ is CR^{G}l; and G³ is CR^{G3}.

In certain embodiments, T of Formula (I-A) is of Formula I-3-ii-a, wherein R^{G4} is hydrogen; and G¹ and G³ are each CH.

In certain embodiments, T of Formula (I-A) is of Formula I-3-ii, wherein H¹ is NR^{H1′}; and H² is NRH²′.

In certain embodiments, T of Formula (I-A) is of Formula I-3-ii, wherein H¹ is NMe; and H² is NMe.

In certain embodiments, T of Formula (I-A) is of Formula I-3-ii, wherein G¹ is N; G² is CR^{G2}; G³ is

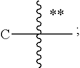

and G⁴ is CR^{G4}.

In certain embodiments, T of Formula (I-A) is of Formula I-3-ii, wherein G¹ is CR^{G1}; G² is

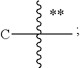

G³ is CR^{G3}; and G⁴ is N.

In certain embodiments, T of Formula (I-A) is of Formula I-3-ii, wherein G¹ is N; G² is CH; G³ is

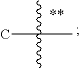

### and G⁴ is CH.

In certain embodiments, T of Formula (I-A) is of Formula I-3-ii, wherein G¹ is CH; G² is

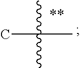

G³ is CH; and G⁴ is N.

In certain embodiments, T of Formula (I-A) is

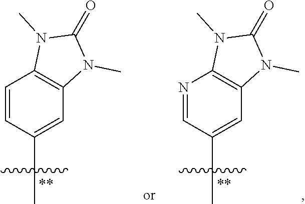 [SMILES: *c1ccc2c(c1)n(C)c(=O)n2C ; *c1cnc2c(c1)n(C)c(=O)n2C]

herein

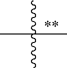

denotes attachment to Cy¹.

In certain embodiments, T of Formula (I-A) is of Formula I-3-vi-a.

In certain embodiments, T of Formula (I-A) is of Formula I-3-vi-a, wherein Q¹ is NR^{Q1}; [symbol] is a double bond; Q² is CR^{Q2}; and Q³ is CR^{Q3}.

In certain embodiments, T of Formula (I-A) is of Formula I-3-vi-a, wherein Q¹ is NMe; [symbol] is a double bond; Q² is CH; and Q³ is CMe.

In certain embodiments, T of Formula (I-A) is of Formula I-3-vi-a, wherein S¹ is N or CR^{S1}; S² is CR^{S2}; S³ is N or CR^{S3}; and S⁴ is

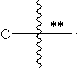

In certain embodiments, T of Formula (I-A) is of Formula I-3-vi-a, wherein S¹ is N; S² is CR^{S2}; S³ is CR^{S3}; and S⁴ is

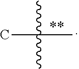

In certain embodiments, T of Formula (I-A) is of Formula I-3-vi-a, wherein S¹ is CR^{S1}; S² is CR^{S2}; S³ is N; and S⁴ is

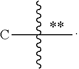

In certain embodiments T of Formula (I-A) is of Formula I-3-vi-a, wherein S¹ is CR^{S1}; S² is CR^{S2}; S³ is CR^{S3}; and S⁴ is

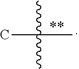

In certain embodiments, T of Formula (I-A) is

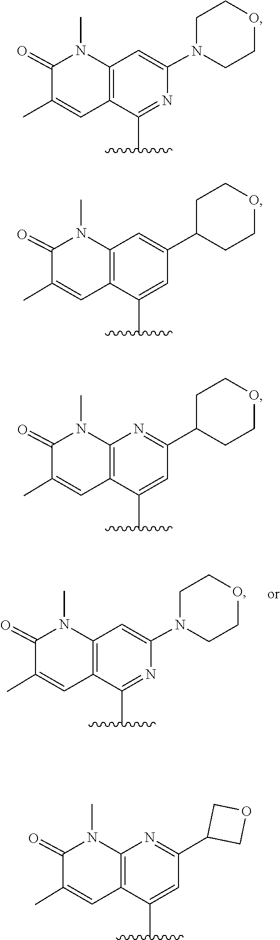 [SMILES: *c1nc(N2CCOCC2)cc2c1cc(C)c(=O)n2C ; *c1cc(C2CCOCC2)cc2c1cc(C)c(=O)n2C ; *c1cc(C2CCOCC2)nc2c1cc(C)c(=O)n2C ; *c1nc(N2CCOCC2)cc2c1cc(C)c(=O)n2C ; *c1cc(C2COC2)nc2c1cc(C)c(=O)n2C]

wherein

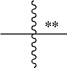

denotes attachment to L″.

In certain embodiments, Cy¹ of Formula (I-A) is

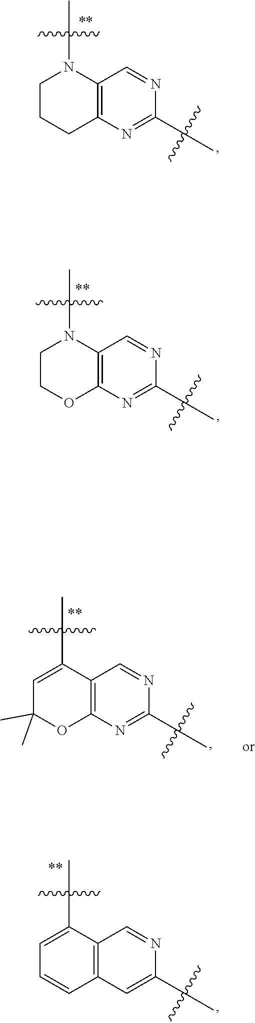 [SMILES: *c1ncc2c(n1)CCCN2* ; *c1ncc2c(n1)OCCN2* ; *C1=CC(C)(C)Oc2nc(*)ncc21 ; *c1cc2cccc(*)c2cn1]

wherein

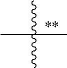

denotes attachment to T.

In certain embodiments, the compound of Formula I-A is of Formula I-A-i or I-A-ii:

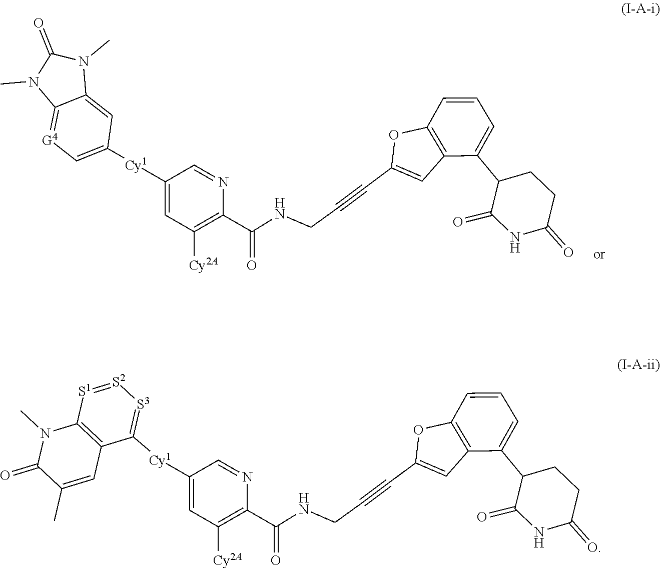

In certain embodiments, the compound of Formula I-A is of Formula I-A′ or IA″

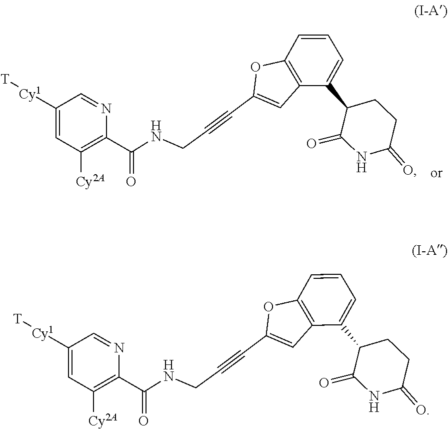

In certain embodiments, the compound of Formula I-A is of Formula I-A′-i, I-A′-ii, or I-A″-ii:

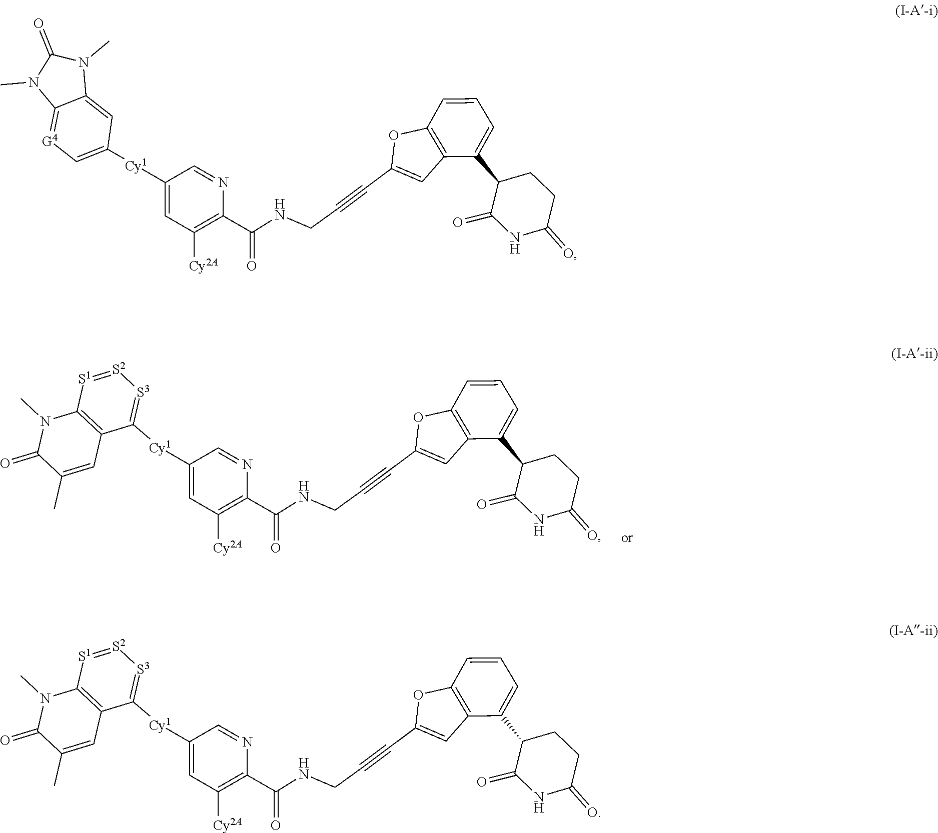

Embodiments of the variables in any of the Formulae described herein, e.g., Formulae I, I-1, I-1-i, I-1-ii, I-1-ii-1, I-1-ii-2, I-1-ii-3, I-1-iii, I-1-iii-1, I-2, I-3-i, I-3-ii, I-3-iii, I-3-iv, I-3-v, and I-3-vi, as applicable, are described below. Any of the variables can be any moiety as described in the embodiments below. In addition, the combination of any moieties described for any of the variables, as applicable, with any moieties described for any of the remaining variables, is also contemplated.

Without wishing to be limited by this statement, while various options for variables are described herein, it is understood that the present disclosure intends to encompass operable embodiments having combinations of the options. The disclosure may be interpreted as excluding the non-operable embodiments caused by certain combinations of the options.

When a range of values is listed, each discrete value and sub-range within the range are also contemplated. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

In certain embodiments, the compound is selected from the compounds in Tables 1 and 2 and pharmaceutically acceptable salts thereof.

In certain embodiments, the compound is selected from the compounds in Tables 1 and 2.

In certain embodiments, the compound is selected from the compounds in Table 1 and pharmaceutically acceptable salts thereof.

In certain embodiments, the compound is selected from the compounds in Table 1.

In certain embodiments, the compound is selected from the compounds in Table 2 and pharmaceutically acceptable salts thereof.

In certain embodiments, the compound is selected from the compounds in Table 2.

TABLE 1

| Com- | | |
|---|---|---|
| pound | | |
| No. | Chemical Structure | Chemical Name |
| A1 | 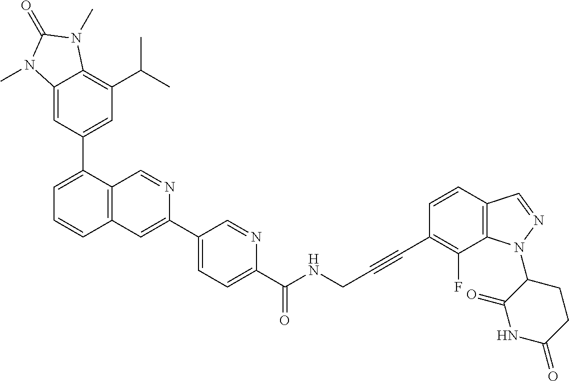 [SMILES: CC(C)c1cc(-c2cccc3cc(-c4ccc(C(=O)NCC#Cc5ccc6cnn(C7CCC(=O)NC7=O)c6c5F)nc4)ncc23)cc2c1n(C)c(=O)n2C] | N-(3-(1-(2,6-dioxo- piperidin-3-yl)-7- fluoro-1H-indazol- 6-yl)prop- 2-yn-1-yl)-5-(8-(7- isopropyl-1,3-dimethyl- 2-oxo-2,3-dihydro-1H- benzo[d]imidazol-5- yl)isoquinolin-3- yl)picolinamide |
| A2 | 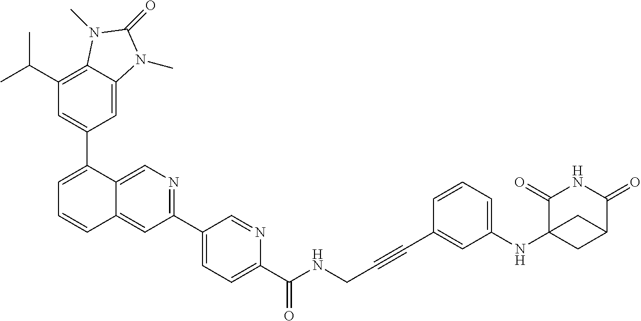 [SMILES: CC(C)c1cc(-c2cccc3cc(-c4ccc(C(=O)NCC#Cc5cccc(NC67CC(C6)C(=O)NC7=O)c5)nc4)ncc23)cc2c1n(C)c(=O)n2C] | N-(3-(3-((2,4-dioxo-3- azabicyclo[3.1.1]heptan-1- yl)amino)phenyl)prop- 2-yn-1-yl)-5-(8-(7- isopropyl-1,3-dimethyl- 2-oxo-2,3-dihydro- 1H-benzo[d]imidazol-5- yl)isoquinolin-3- yl)picolinamide |
| A3 | 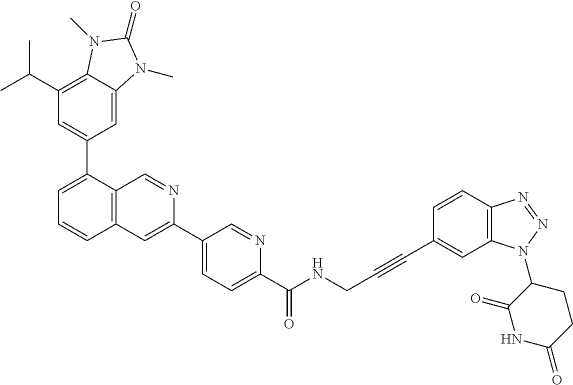 [SMILES: CC(C)c1cc(-c2cccc3cc(-c4ccc(C(=O)NCC#Cc5ccc6nnn(C7CCC(=O)NC7=O)c6c5)nc4)ncc23)cc2c1n(C)c(=O)n2C] | N-(3-(1-(2,6-dioxo- piperidin- 3-yl)-1H-benzo[d] [1,2,3]triazol-6-yl) prop-2-yn-1-yl)-5- (8-(7-isopropyl-1,3- dimethyl-2-oxo- 2,3-dihydro- 1H-benzo[d]imidazol-5- yl)isoquinolin-3- yl)picolinamide |
| A4 | 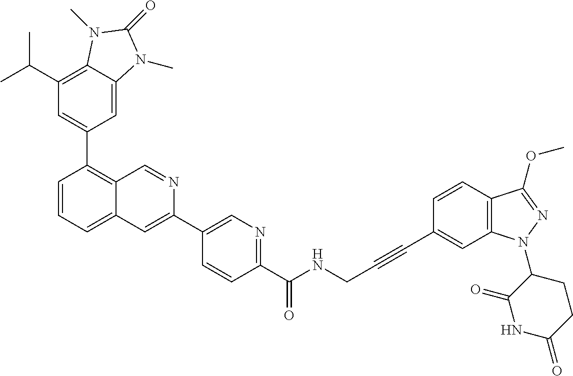 [SMILES: COc1nn(C2CCC(=O)NC2=O)c2cc(C#CCNC(=O)c3ccc(-c4cc5cccc(-c6cc(C(C)C)c7c(c6)n(C)c(=O)n7C)c5cn4)cn3)ccc12] | N-(3-(1-(2,6-dioxo- piperidin-3-yl)- 3-methoxy-1H- indazol-6-yl)prop-2-yn-1- yl)-5-(8-(7-isopropyl-1,3- dimethyl-2-oxo- 2,3-dihydro- 1H-benzo[d]imidazol-5- yl)isoquinolin-3- yl)picolinamide |
| A5 | 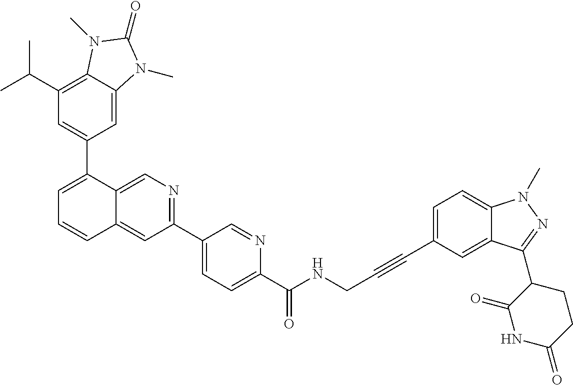 [SMILES: CC(C)c1cc(-c2cccc3cc(-c4ccc(C(=O)NCC#Cc5ccc6c(c5)c(C5CCC(=O)NC5=O)nn6C)nc4)ncc23)cc2c1n(C)c(=O)n2C] | N-(3-(3-(2,6- dioxopiperidin- 3-yl)-1-methyl-1H- indazol-5-yl)prop-2-yn- 1-yl)-5-(8-(7-isopropyl- 1,3-dimethyl- 2-oxo-2,3-dihydro- 1H-benzo[d]imidazol-5- yl)isoquinolin-3- yl)picolinamide |
| A6 | 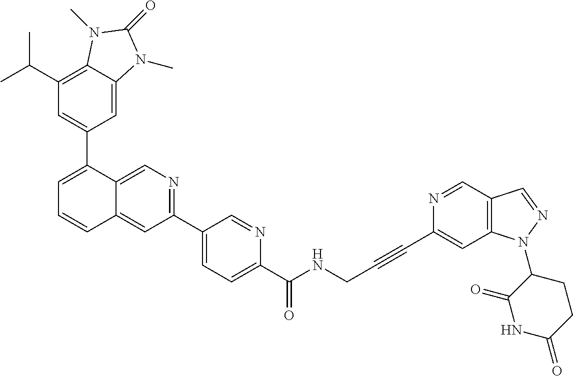 [SMILES: CC(C)c1cc(-c2cccc3cc(-c4ccc(C(=O)NCC#Cc5cc6c(cn5)cnn6C5CCC(=O)NC5=O)nc4)ncc23)cc2c1n(C)c(=O)n2C] | N-(3-(1-(2,6- dioxopiperidin- 3-yl)-1H-pyrazolo[4,3-c] pyridin-6-yl)prop-2-yn-1- yl)-5-(8-(7-isopropyl-1,3- dimethyl-2-oxo- 2,3-dihydro- 1H-benzo[d]imidazol-5- yl)isoquinolin-3- yl)picolinamide |
| A7 | 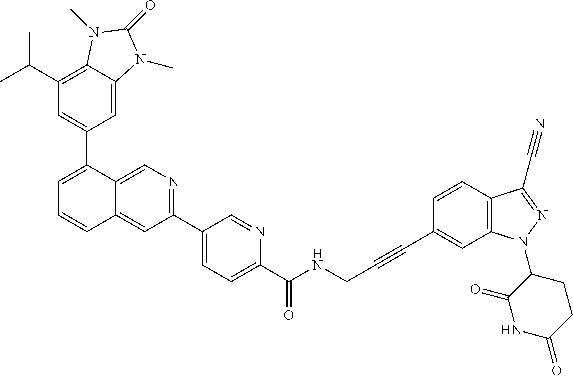 [SMILES: CC(C)c1cc(-c2cccc3cc(-c4ccc(C(=O)NCC#Cc5ccc6c(C#N)nn(C7CCC(=O)NC7=O)c6c5)nc4)ncc23)cc2c1n(C)c(=O)n2C] | N-(3-(3-cyano-1-(2,6- dioxopiperidin-3-yl)-1H- indazol-6-yl)prop-2-yn-1- yl)-5-(8-(7-isopropyl- 1,3-dimethyl-2-oxo-2,3- dihydro-1H-benzo [d]imidazol-5- yl)isoquinolin-3- yl)picolinamide |
| A8 | 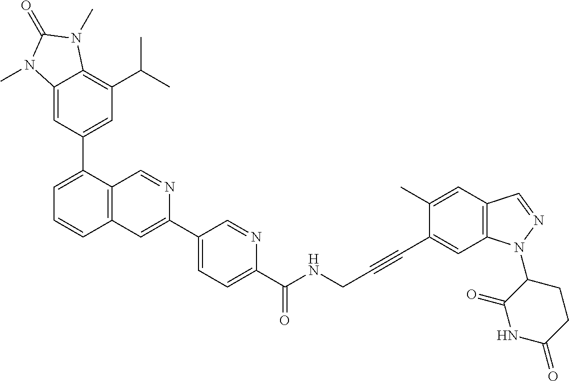 [SMILES: Cc1cc2cnn(C3CCC(=O)NC3=O)c2cc1C#CCNC(=O)c1ccc(-c2cc3cccc(-c4cc(C(C)C)c5c(c4)n(C)c(=O)n5C)c3cn2)cn1] | N-(3-(1-(2,6- dioxopiperidin- 3-yl)-5-methyl-1H- indazol-6-yl) prop-2-yn-1-yl)- 5-(8-(7-isopropyl-1,3- dimethyl-2-oxo-2,3- dihydro-1H-benzo [d]imidazol-5- yl)isoquinolin-3- yl)picolinamide |
| A9 | 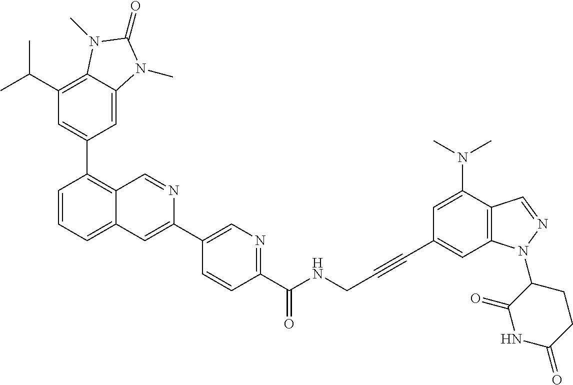 [SMILES: CC(C)c1cc(-c2cccc3cc(-c4ccc(C(=O)NCC#Cc5cc(N(C)C)c6cnn(C7CCC(=O)NC7=O)c6c5)nc4)ncc23)cc2c1n(C)c(=O)n2C] | N-(3-(4-(dimethylamino)- 1-(2,6-dioxopiperidin- 3-yl)-1H-indazol-6- yl)prop-2-yn-1-yl)-5- (8-(7-isopropyl- 1,3-dimethyl- 2-oxo-2,3-dihydro-1H- benzo[d]imidazol-5- yl)isoquinolin-3- yl)picolinamide |
| A10 | 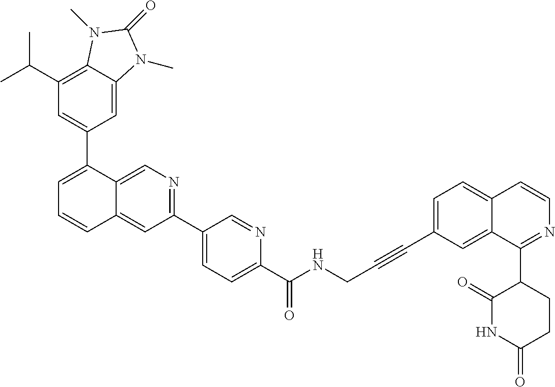 [SMILES: CC(C)c1cc(-c2cccc3cc(-c4ccc(C(=O)NCC#Cc5ccc6ccnc(C7CCC(=O)NC7=O)c6c5)nc4)ncc23)cc2c1n(C)c(=O)n2C] | N-(3-(1-(2,6- dioxopiperidin- 3-yl)isoquinolin-7- yl)prop-2-yn-1-yl)-5-(8- (7-isopropyl-1,3- dimethyl-2-oxo-2,3- dihydro-1H-benzo [d]imidazol-5- yl)isoquinolin-3- yl)picolinamide |
| A11 | 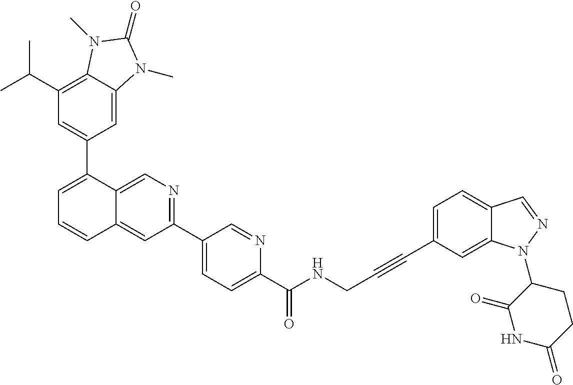 [SMILES: CC(C)c1cc(-c2cccc3cc(-c4ccc(C(=O)NCC#Cc5ccc6cnn(C7CCC(=O)NC7=O)c6c5)nc4)ncc23)cc2c1n(C)c(=O)n2C] | N-(3-(1-(2,6-dioxo- piperidin-3-yl)-1H- indazol-6-yl)prop- 2-yn-1-yl)-5-(8-(7- isopropyl-1,3- dimethyl-2-oxo-2,3- dihydro-1H-benzo [d]imidazol-5- yl)isoquinolin-3- yl)picolinamide |
| A12 | 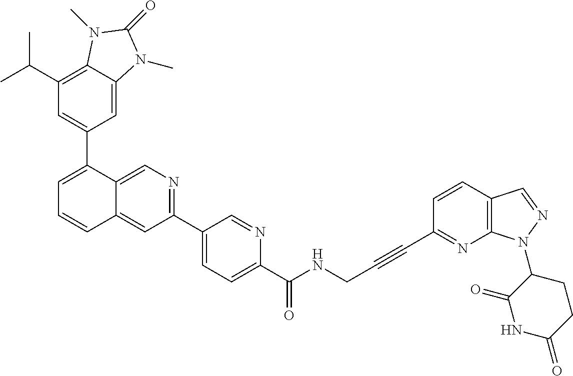 [SMILES: CC(C)c1cc(-c2cccc3cc(-c4ccc(C(=O)NCC#Cc5ccc6cnn(C7CCC(=O)NC7=O)c6n5)nc4)ncc23)cc2c1n(C)c(=O)n2C] | N-(3-(1-(2,6- dioxopiperidin- 3-yl)-1H-pyrazolo[3,4-b] pyridin-6-yl)prop-2-yn-1- yl)-5-(8-(7-isopropyl-1,3- dimethyl-2-oxo- 2,3-dihydro- 1H-benzo[d]imidazol-5- yl)isoquinolin-3- yl)picolinamide |
| A13 | 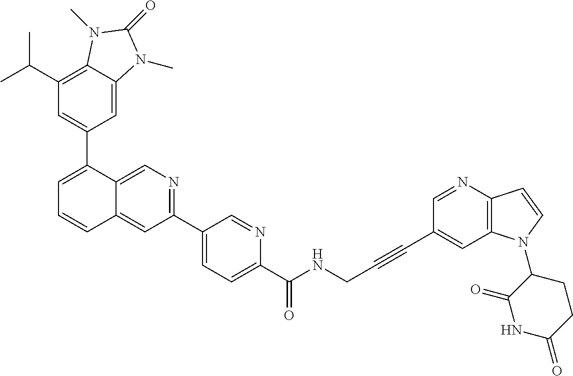 [SMILES: CC(C)c1cc(-c2cccc3cc(-c4ccc(C(=O)NCC#Cc5cnc6ccn(C7CCC(=O)NC7=O)c6c5)nc4)ncc23)cc2c1n(C)c(=O)n2C] | N-(3-(1-(2,6- dioxopiperidin- 3-yl)-1H-pyrrolo[3,2-b] pyridin-6-yl)prop-2-yn-1- yl)-5-(8-(7-isopropyl-1,3- dimethyl-2-oxo- 2,3-dihydro- 1H-benzo[d]imidazol-5- yl)isoquinolin-3- yl)picolinamide |
| A14 | 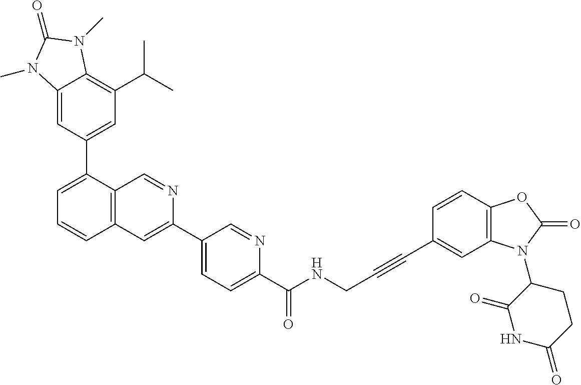 [SMILES: CC(C)c1cc(-c2cccc3cc(-c4ccc(C(=O)NCC#Cc5ccc6oc(=O)n(C7CCC(=O)NC7=O)c6c5)nc4)ncc23)cc2c1n(C)c(=O)n2C] | N-(3-(3-(2,6- dioxopiperidin- 3-yl)-2-oxo-2,3- dihydrobenzo [d]oxazol-5-yl)prop-2- yn-1-yl)-5-(8- (7-isopropyl- 1,3-dimethyl-2- oxo-2,3-dihydro- 1H-benzo[d]imidazol- 5-yl)isoquinolin-3- yl)picolinamide |
| A15 | 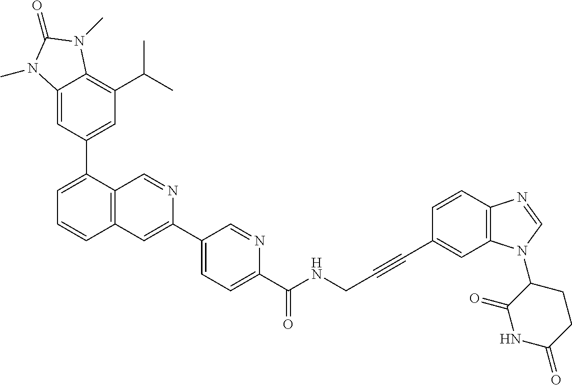 [SMILES: CC(C)c1cc(-c2cccc3cc(-c4ccc(C(=O)NCC#Cc5ccc6ncn(C7CCC(=O)NC7=O)c6c5)nc4)ncc23)cc2c1n(C)c(=O)n2C] | N-(3-(1-(2,6- dioxopiperidin- 3-yl)-1H- benzo[d]imidazol- 6-yl)prop-2-yn-1-yl)-5- (8-(7-isopropyl-1,3- dimethyl-2-oxo- 2,3-dihydro- 1H-benzo[d]imidazol-5- yl)isoquinolin-3- yl)picolinamide |
| A16 | 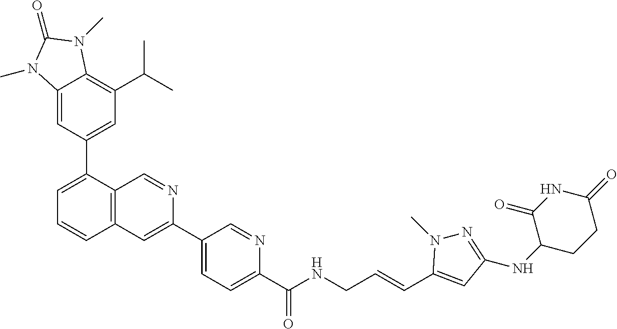 [SMILES: CC(C)c1cc(-c2cccc3cc(-c4ccc(C(=O)NC/C=C/c5cc(NC6CCC(=O)NC6=O)nn5C)nc4)ncc23)cc2c1n(C)c(=O)n2C] | (E)-N-(3-(3-((2,6- dioxopiperidin- 3-yl)amino)-1-methyl-1H- pyrazol-5-yl)allyl)-5- (8-(7-isopropyl-1,3- dimethyl-2-oxo-2,3- dihydro-1H- benzo[d]imidazol-5-yl) isoquinolin-3- yl)picolinamide |
| A17 | 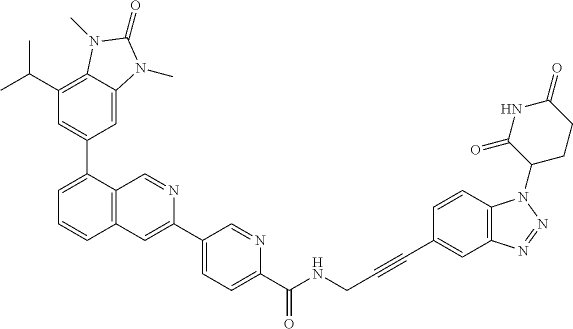 [SMILES: CC(C)c1cc(-c2cccc3cc(-c4ccc(C(=O)NCC#Cc5ccc6c(c5)nnn6C5CCC(=O)NC5=O)nc4)ncc23)cc2c1n(C)c(=O)n2C] | N-(3-(1-(2,6- dioxopiperidin- 3-yl)-1H-benzo[d][1,2,3] triazol-5-yl)prop-2-yn-1- yl)-5-(8-(7-isopropyl-1,3- dimethyl-2-oxo- 2,3-dihydro- 1H-benzo[d]imidazol-5- yl)isoquinolin-3- yl)picolinamide |
| A18 | 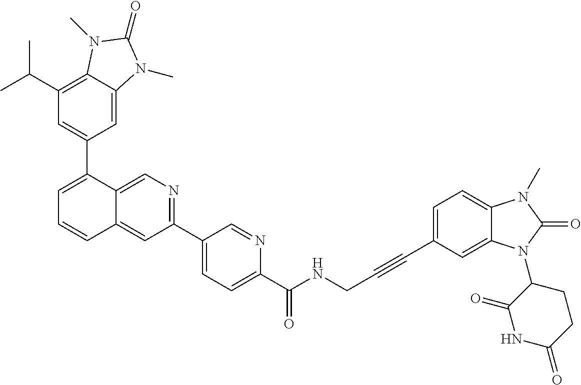 [SMILES: CC(C)c1cc(-c2cccc3cc(-c4ccc(C(=O)NCC#Cc5ccc6c(c5)n(C5CCC(=O)NC5=O)c(=O)n6C)nc4)ncc23)cc2c1n(C)c(=O)n2C] | N-(3-(3-(2,6- dioxopiperidin- 3-yl)-1-methyl-2-oxo- 2,3-dihydro-1H-benzo[d] imidazol-5-yl) prop-2-yn-1- yl)-5-(8-(7-isopropyl- 1,3-dimethyl-2-oxo-2,3- dihydro-1H-benzo [d]imidazol-5- yl)isoquinolin-3- yl)picolinamide |
| A19 | 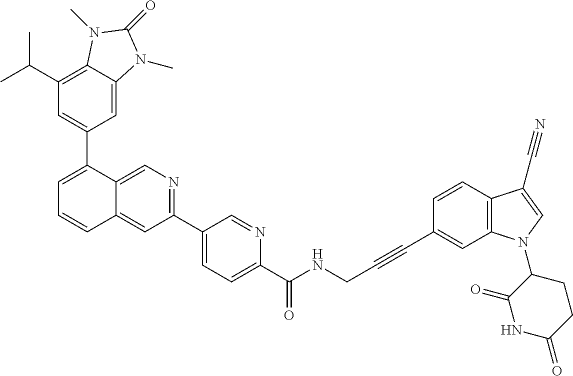 [SMILES: CC(C)c1cc(-c2cccc3cc(-c4ccc(C(=O)NCC#Cc5ccc6c(C#N)cn(C7CCC(=O)NC7=O)c6c5)nc4)ncc23)cc2c1n(C)c(=O)n2C] | N-(3-(3-cyano-1- (2,6-dioxopiperidin- 3-yl)-1H-indol- 6-yl)prop-2-yn-1- yl)-5-(8-(7- isopropyl-1,3- dimethyl-2-oxo-2,3- dihydro-1H-benzo [d]imidazol-5- yl)isoquinolin-3- yl)picolinamide |
| A20 | 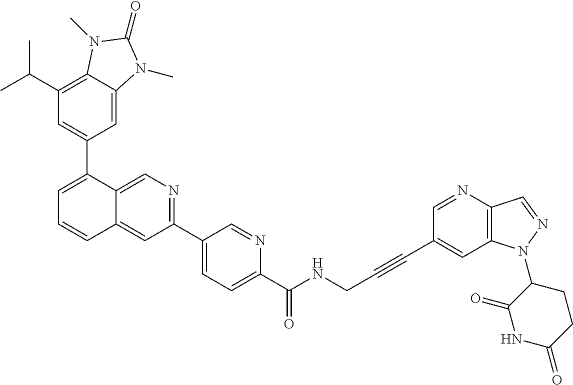 [SMILES: CC(C)c1cc(-c2cccc3cc(-c4ccc(C(=O)NCC#Cc5cnc6cnn(C7CCC(=O)NC7=O)c6c5)nc4)ncc23)cc2c1n(C)c(=O)n2C] | N-(3-(1-(2,6- dioxopiperidin- 3-yl)-1H-pyrazolo[4,3-b] pyridin-6-yl)prop-2-yn-1- yl)-5-(8-(7-isopropyl-1,3- dimethyl-2-oxo- 2,3-dihydro- 1H-benzo[d]imidazol-5- yl)isoquinolin-3- yl)picolinamide |
| A21 | 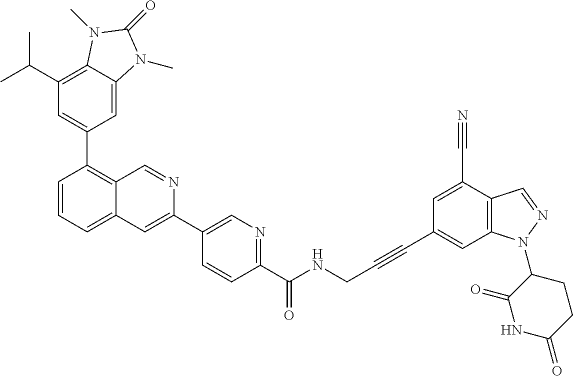 [SMILES: CC(C)c1cc(-c2cccc3cc(-c4ccc(C(=O)NCC#Cc5cc(C#N)c6cnn(C7CCC(=O)NC7=O)c6c5)nc4)ncc23)cc2c1n(C)c(=O)n2C] | N-(3-(4-cyano-1- (2,6-dioxopiperidin- 3-yl)-1H-indazol- 6-yl)prop-2-yn-1- yl)-5-(8-(7- isopropyl-1,3- dimethyl-2-oxo-2,3- dihydro-1H-benzo [d]imidazol-5- yl)isoquinolin-3- yl)picolinamide |
| A22 | 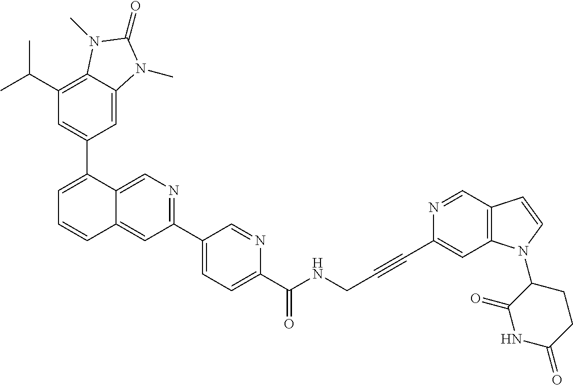 [SMILES: CC(C)c1cc(-c2cccc3cc(-c4ccc(C(=O)NCC#Cc5cc6c(ccn6C6CCC(=O)NC6=O)cn5)nc4)ncc23)cc2c1n(C)c(=O)n2C] | N-(3-(1-(2,6-dioxo- piperidin-3-yl)-1H- pyrrolo[3,2-c] pyridin-6-yl)prop- 2-yn-1-yl)-5-(8- (7-isopropyl-1,3- dimethyl-2-oxo- 2,3-dihydro-1H- benzo[d]imidazol-5- yl)isoquinolin-3- yl)picolinamide |
| A23 | 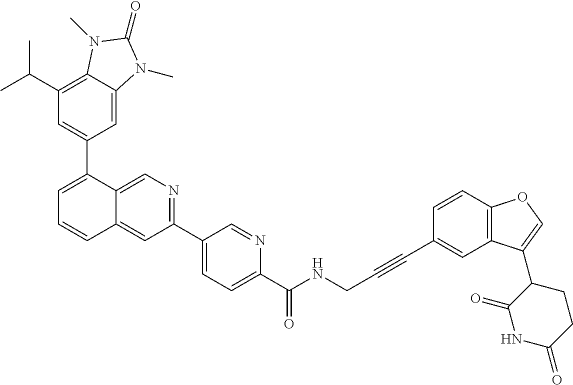 [SMILES: CC(C)c1cc(-c2cccc3cc(-c4ccc(C(=O)NCC#Cc5ccc6occ(C7CCC(=O)NC7=O)c6c5)nc4)ncc23)cc2c1n(C)c(=O)n2C] | N-(3-(3-(2,6-dioxo- piperidin-3-yl)benzo- furan-5-yl)prop- 2-yn-1-yl)-5-(8-(7- isopropyl-1,3- dimethyl-2-oxo-2,3- dihydro-1H-benzo [d]imidazol-5- yl)isoquinolin-3- yl)picolinamide |
| A24 | 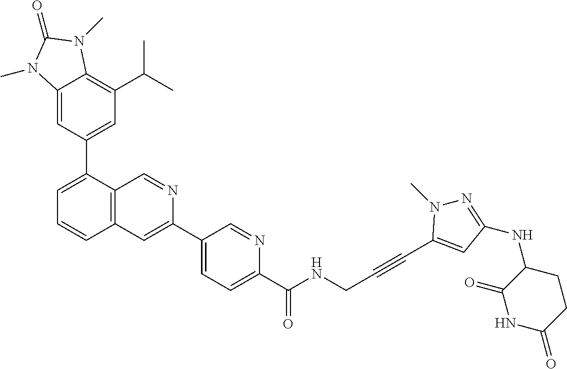 [SMILES: CC(C)c1cc(-c2cccc3cc(-c4ccc(C(=O)NCC#Cc5cc(NC6CCC(=O)NC6=O)nn5C)nc4)ncc23)cc2c1n(C)c(=O)n2C] | N-(3-(3-((2,6- dioxopiperidin- 3-yl)amino)-1-methyl-1H- pyrazol-5-yl)prop-2-yn-1- yl)-5-(8-(7-isopropyl-1,3- dimethyl-2-oxo- 2,3-dihydro- 1H-benzo[d]imidazol-5- yl)isoquinolin-3- yl)picolinamide |
| A25 | 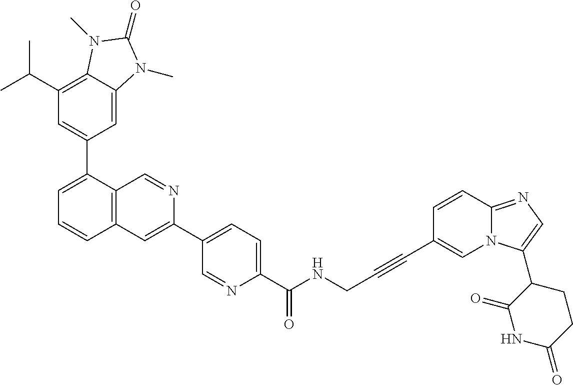 [SMILES: CC(C)c1cc(-c2cccc3cc(-c4ccc(C(=O)NCC#Cc5ccc6ncc(C7CCC(=O)NC7=O)n6c5)nc4)ncc23)cc2c1n(C)c(=O)n2C] | N-(3-(3-(2,6-dioxo- piperidin-3-yl)imidazo [1,2-a]pyridin-6-yl) prop-2-yn-1-yl)-5-(8-(7- isopropyl-1,3-dimethyl-2- oxo-2,3-dihydro-1H- benzo[d]imidazol-5- yl)isoquinolin-3- yl)picolinamide |
| A26 | 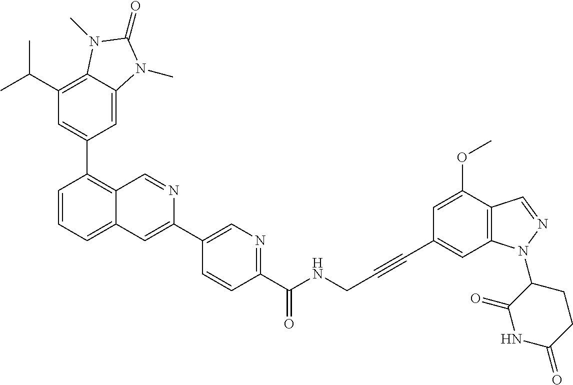 [SMILES: COc1cc(C#CCNC(=O)c2ccc(-c3cc4cccc(-c5cc(C(C)C)c6c(c5)n(C)c(=O)n6C)c4cn3)cn2)cc2c1cnn2C1CCC(=O)NC1=O] | N-(3-(1-(2,6- dioxopiperidin- 3-yl)-4-methoxy-1H- indazol-6-yl)prop-2-yn-1- yl)-5-(8-(7-isopropyl-1,3- dimethyl-2-oxo- 2,3-dihydro-1H- benzo[d]imidazol-5- yl)isoquinolin-3- yl)picolinamide |
| A27 | 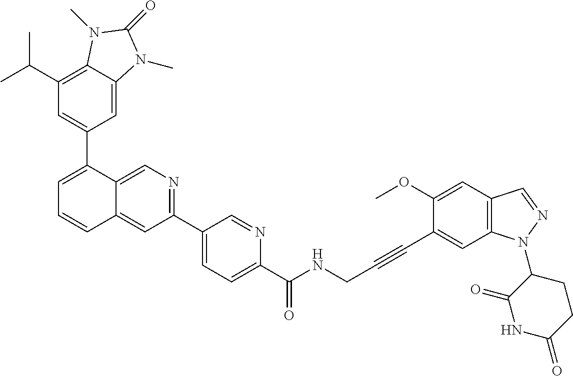 [SMILES: COc1cc2cnn(C3CCC(=O)NC3=O)c2cc1C#CCNC(=O)c1ccc(-c2cc3cccc(-c4cc(C(C)C)c5c(c4)n(C)c(=O)n5C)c3cn2)cn1] | N-(3-(1-(2,6- dioxopiperidin- 3-yl)-5-methoxy-1H- indazol-6-yl)prop-2-yn-1- yl)-5-(8-(7-isopropyl-1,3- dimethyl-2-oxo- 2,3-dihydro- 1H-benzo[d]imidazol-5- yl)isoquinolin-3- yl)picolinamide |
| A28 | 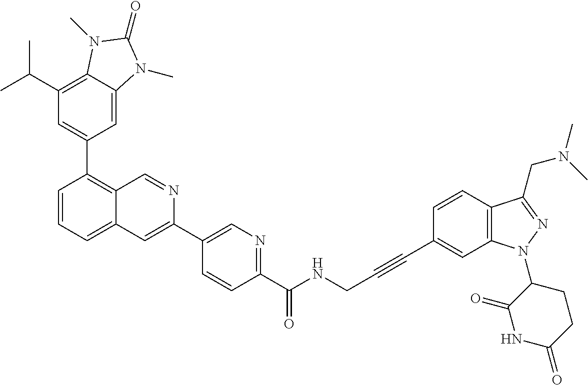 [SMILES: CC(C)c1cc(-c2cccc3cc(-c4ccc(C(=O)NCC#Cc5ccc6c(CN(C)C)nn(C7CCC(=O)NC7=O)c6c5)nc4)ncc23)cc2c1n(C)c(=O)n2C] | N-(3-(3-((dimethylamino) methyl)-1-(2,6- dioxopiperidin- 3-yl)-1H-indazol-6- yl)prop-2-yn-1-yl)-5- (8-(7-isopropyl-1,3- dimethyl-2-oxo-2,3- dihydro-1H-benzo [d]imidazol- 5-yl)isoquinolin-3- yl)picolinamide |
| A29 | 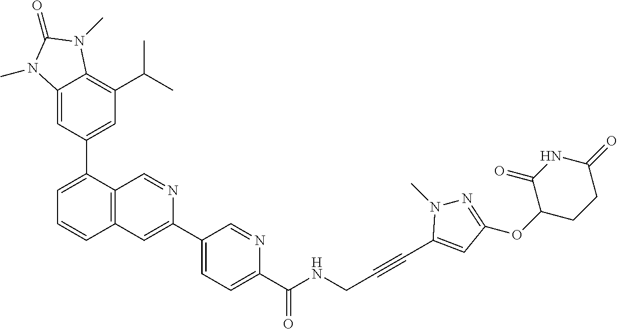 [SMILES: CC(C)c1cc(-c2cccc3cc(-c4ccc(C(=O)NCC#Cc5cc(OC6CCC(=O)NC6=O)nn5C)nc4)ncc23)cc2c1n(C)c(=O)n2C] | N-(3-(3-((2,6- dioxopiperidin- 3-yl)oxy)-1-methyl-1H- pyrazol-5-yl)prop- 2-yn-1-yl)- 5-(8-(7-isopropyl-1,3- dimethyl-2-oxo- 2,3-dihydro- 1H-benzo[d]imidazol-5- yl)isoquinolin-3- yl)picolinamide |
| A30 | 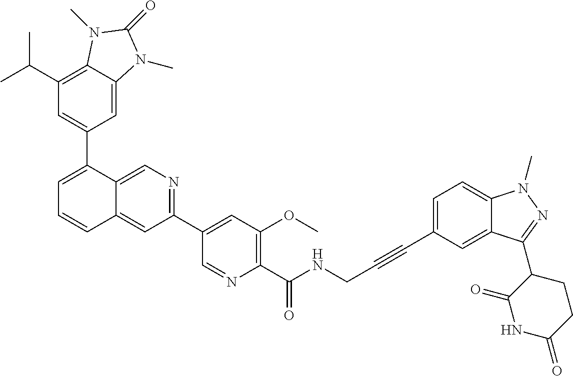 [SMILES: COc1cc(-c2cc3cccc(-c4cc(C(C)C)c5c(c4)n(C)c(=O)n5C)c3cn2)cnc1C(=O)NCC#Cc1ccc2c(c1)c(C1CCC(=O)NC1=O)nn2C] | N-(3-(3-(2,6- dioxopiperidin- 3-yl)-1-methyl- 1H-indazol- 5-yl)prop-2-yn-1-yl)- 5-(8-(7-isopropyl-1,3- dimethyl-2-oxo- 2,3-dihydro- 1H-benzo[d]imidazol-5- yl)isoquinolin-3-yl)-3- methoxypicolinamide |
| A31 | 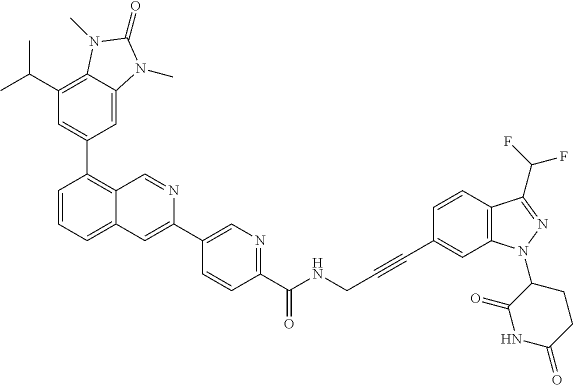 [SMILES: CC(C)c1cc(-c2cccc3cc(-c4ccc(C(=O)NCC#Cc5ccc6c(C(F)F)nn(C7CCC(=O)NC7=O)c6c5)nc4)ncc23)cc2c1n(C)c(=O)n2C] | N-(3-(3-(difluoromethyl)- 1-(2,6-dioxopiperidin- 3-yl)-1H-indazol- 6-yl)prop-2-yn-1- yl)-5-(8-(7-isopropyl- 1,3-dimethyl-2- oxo-2,3-dihydro-1H- benzo[d]imidazol- 5-yl)isoquinolin- 3-yl)picolinamide |
| A32 | 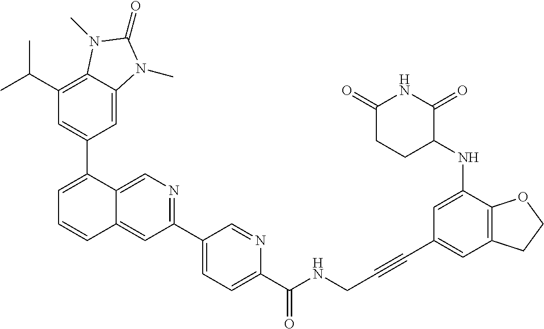 [SMILES: CC(C)c1cc(-c2cccc3cc(-c4ccc(C(=O)NCC#Cc5cc6c(c(NC7CCC(=O)NC7=O)c5)OCC6)nc4)ncc23)cc2c1n(C)c(=O)n2C] | N-(3-(7-((2,6- dioxopiperidin- 3-yl)amino)-2,3-dihydro- benzofuran-5-yl) prop-2-yn-1- yl)-5-(8-(7-isopropyl- 1,3-dimethyl-2-oxo- 2,3-dihydro-1H- benzo[d]imidazol-5- yl)isoquinolin-3- yl)picolinamide |
| A33 | 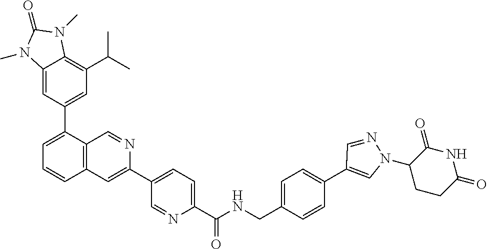 [SMILES: CC(C)c1cc(-c2cccc3cc(-c4ccc(C(=O)NCc5ccc(-c6cnn(C7CCC(=O)NC7=O)c6)cc5)nc4)ncc23)cc2c1n(C)c(=O)n2C] | N-(4-(1-(2,6- dioxopiperidin- 3-yl)-1H-pyrazol-4-yl) benzyl)-5-(8-(7-isopropyl- 1,3-dimethyl-2-oxo- 2,3-dihydro-1H-benzo[d] imidazol-5-yl) isoquinolin-3- yl)picolinamide |
| A34 | 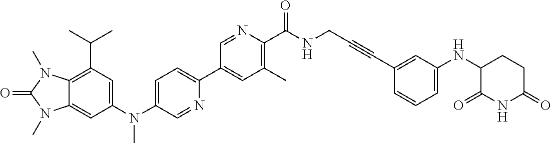 [SMILES: Cc1cc(-c2ccc(N(C)c3cc(C(C)C)c4c(c3)n(C)c(=O)n4C)cn2)cnc1C(=O)NCC#Cc1cccc(NC2CCC(=O)NC2=O)c1] | N-(3-(3-((2,6- dioxopiperidin-3-yl) amino)phenyl)prop-2- yn-1-yl)-5-((7-isopropyl- 1,3-dimethyl-2-oxo-2,3- dihydro-1H-benzo [d]imidazol-5- yl)(methyl)amino)- 5′-methyl-[2,3′- |
| | | bipyridine]-6′- |
| | | carboxamide |
| A35 | 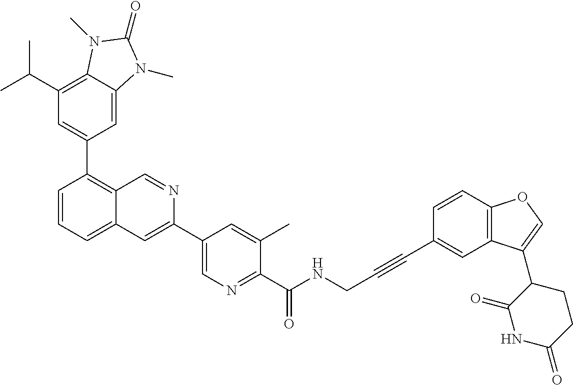 [SMILES: Cc1cc(-c2cc3cccc(-c4cc(C(C)C)c5c(c4)n(C)c(=O)n5C)c3cn2)cnc1C(=O)NCC#Cc1ccc2occ(C3CCC(=O)NC3=O)c2c1] | N-(3-(3-(2,6- dioxopiperidin-3- yl)benzofuran-5-yl)prop- 2-yn-1-yl)-5-(8-(7- isopropyl-1,3-dimethyl- 2-oxo-2,3-dihydro-1H- benzo[d]imidazol-5- yl)isoquinolin-3-yl)-3- methylpicolinamide |
| A36 | 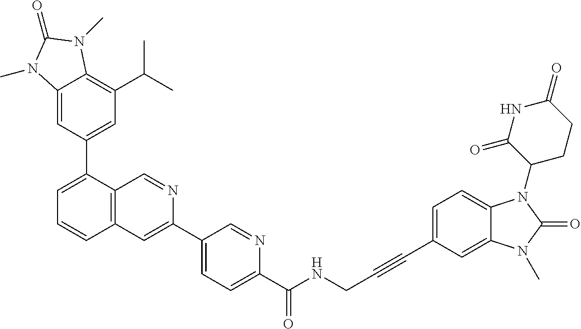 [SMILES: CC(C)c1cc(-c2cccc3cc(-c4ccc(C(=O)NCC#Cc5ccc6c(c5)n(C)c(=O)n6C5CCC(=O)NC5=O)nc4)ncc23)cc2c1n(C)c(=O)n2C] | N-(3-(1-(2,6- dioxopiperidin- 3-yl)-3-methyl-2-oxo- 2,3-dihydro-1H- benzo[d]imidazol-5-yl) prop-2-yn-1-yl)-5-(8-(7- isopropyl-1,3-dimethyl- 2-oxo-2,3-dihydro-1H- benzo[d]imidazol-5- yl)isoquinolin-3- yl)picolinamide |
| A37 | 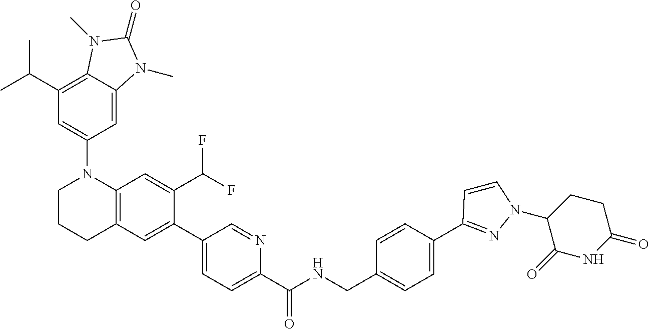 [SMILES: CC(C)c1cc(N2CCCc3cc(-c4ccc(C(=O)NCc5ccc(-c6ccn(C7CCC(=O)NC7=O)n6)cc5)nc4)c(C(F)F)cc32)cc2c1n(C)c(=O)n2C] | 5-(7-(difluoromethyl)- 1-(7-isopropyl-1,3- dimethyl-2-oxo- 2,3-dihydro-1H- benzo[d]imidazol- 5-yl)-1,2,3,4- tetrahydroquinolin- 6-yl)-N-(4-(1-(2,6- dioxopiperidin-3- yl)-1H-pyrazol-3- yl)benzyl)picolinamide |
| A38 | 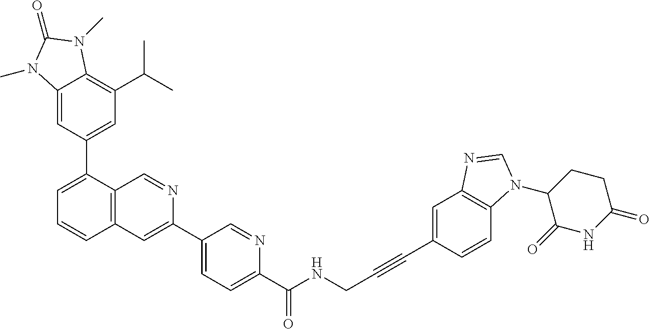 [SMILES: CC(C)c1cc(-c2cccc3cc(-c4ccc(C(=O)NCC#Cc5ccc6c(c5)ncn6C5CCC(=O)NC5=O)nc4)ncc23)cc2c1n(C)c(=O)n2C] | N-(3-(1-(2,6- dioxopiperidin- 3-yl)-1H-benzo[d] imidazol-5-yl)prop-2-yn- 1-yl)-5-(8-(7-isopropyl- 1,3-dimethyl-2-oxo-2,3- dihydro-1H-benzo [d]imidazol-5- yl)isoquinolin-3- yl)picolinamide |
| A39 | 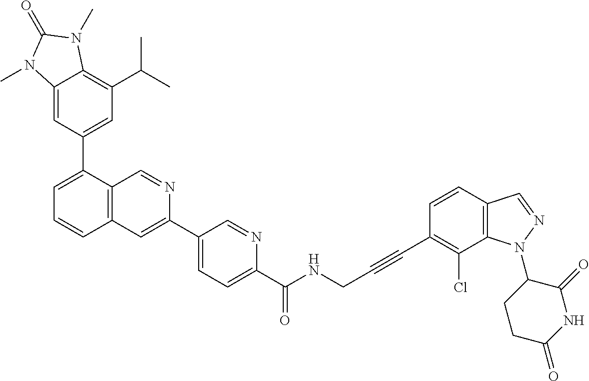 [SMILES: CC(C)c1cc(-c2cccc3cc(-c4ccc(C(=O)NCC#Cc5ccc6cnn(C7CCC(=O)NC7=O)c6c5Cl)nc4)ncc23)cc2c1n(C)c(=O)n2C] | N-(3-(7-chloro-1-(2,6- dioxopiperidin-3-yl)-1H- indazol-6-yl)prop-2-yn-1- yl)-5-(8-(7-isopropyl- 1,3-dimethyl-2-oxo-2,3- dihydro-1H-benzo [d]imidazol-5- yl)isoquinolin-3- yl)picolinamide |
| A40 | 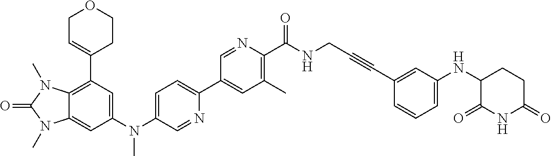 [SMILES: Cc1cc(-c2ccc(N(C)c3cc(C4=CCOCC4)c4c(c3)n(C)c(=O)n4C)cn2)cnc1C(=O)NCC#Cc1cccc(NC2CCC(=O)NC2=O)c1] | 5-((7-(3,6-dihydro-2H- pyran-4-yl)-1,3-dimethyl- 2-oxo-2,3-dihydro- 1H-benzo[d] imidazol-5-yl)(methyl) amino)-N-(3-(3- ((2,6-dioxopiperidin-3- yl)amino)phenyl)prop-2- yn-1-yl)-5′-methyl-[2,3′- bipyridine]-6′- |
| | | carboxamide |
| A41 | 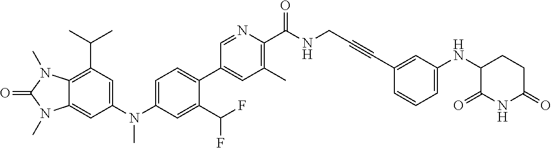 [SMILES: Cc1cc(-c2ccc(N(C)c3cc(C(C)C)c4c(c3)n(C)c(=O)n4C)cc2C(F)F)cnc1C(=O)NCC#Cc1cccc(NC2CCC(=O)NC2=O)c1] | 5-(2-(difluoromethyl)- 4-((7-isopropyl-1,3- dimethyl-2-oxo- 2,3-dihydro-1H- benzo[d]imidazol-5-yl) (methyl)amino)phenyl)- N-(3-(3-((2,6- dioxopiperidin- 3-yl)amino)phenyl)- prop-2-yn-1-yl)-3- |
| | | methylpicolinamide |
| A42 | 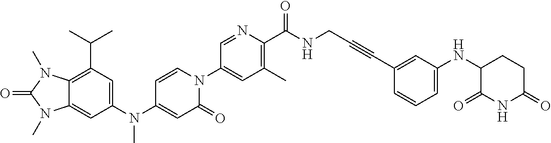 [SMILES: Cc1cc(-n2ccc(N(C)c3cc(C(C)C)c4c(c3)n(C)c(=O)n4C)cc2=O)cnc1C(=O)NCC#Cc1cccc(NC2CCC(=O)NC2=O)c1] | N-(3-(3-((2,6- dioxopiperidin-3-yl) amino)phenyl)prop-2-yn- 1-yl)-4-((7-isopropyl-1,3- dimethyl-2-oxo-2,3- dihydro-1H-benzo [d]imidazol-5- yl)(methyl)amino)-5′- methyl-2-oxo-2H-[1,3′- |
| | | bipyridine]-6′- |
| | | carboxamide |
| A43 | 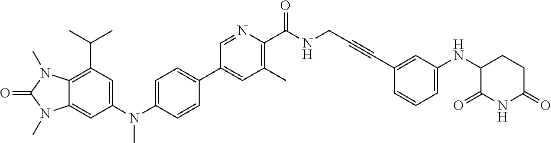 [SMILES: Cc1cc(-c2ccc(N(C)c3cc(C(C)C)c4c(c3)n(C)c(=O)n4C)cc2)cnc1C(=O)NCC#Cc1cccc(NC2CCC(=O)NC2=O)c1] | N-(3-(3-((2,6- dioxopiperidin-3-yl) amino)phenyl)prop-2-yn- 1-yl)-5-(4-((7-isopropyl- 1,3-dimethyl-2-oxo-2,3- dihydro-1H-benzo [d]imidazol-5-yl) (methyl)amino)phenyl)-3- methylpicolinamide |
| A44 | 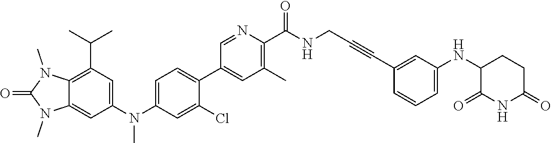 [SMILES: Cc1cc(-c2ccc(N(C)c3cc(C(C)C)c4c(c3)n(C)c(=O)n4C)cc2Cl)cnc1C(=O)NCC#Cc1cccc(NC2CCC(=O)NC2=O)c1] | 5-(2-chloro-4- ((7-isopropyl- 1,3-dimethyl-2-oxo-2,3- dihydro-1H-benzo[d] imidazol-5-yl)(methyl) amino)phenyl)-N-(3-(3- ((2,6-dioxopiperidin-3- yl)amino)phenyl) prop-2-yn-1-yl)-3- methylpicolinamide |
| A45 | 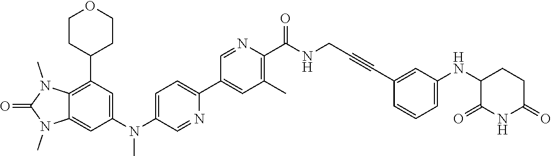 [SMILES: Cc1cc(-c2ccc(N(C)c3cc(C4CCOCC4)c4c(c3)n(C)c(=O)n4C)cn2)cnc1C(=O)NCC#Cc1cccc(NC2CCC(=O)NC2=O)c1] | 5-((1,3-dimethyl-2-oxo- 7-(tetrahydro-2H-pyran-4- yl)-2,3-dihydro-1H- benzo[d]imidazol-5-yl) (methyl)amino)-N-(3- (3-((2,6-dioxopiperidin-3- yl)amino)phenyl)prop-2- yn-1-yl)-5′-methyl-[2,3′- bipyridine]-6′-carboxamide |
| A46 | 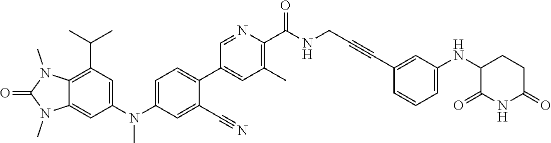 [SMILES: Cc1cc(-c2ccc(N(C)c3cc(C(C)C)c4c(c3)n(C)c(=O)n4C)cc2C#N)cnc1C(=O)NCC#Cc1cccc(NC2CCC(=O)NC2=O)c1] | 5-(2-cyano-4-((7-isopropyl- 1,3-dimethyl-2-oxo-2,3- dihydro-1H-benzo[d] imidazol-5-yl)(methyl) amino)phenyl)-N-(3- (3-((2,6-dioxopiperidin-3- yl)amino)phenyl) prop-2-yn-1- yl)-3-methylpicolinamide |
| A47 | 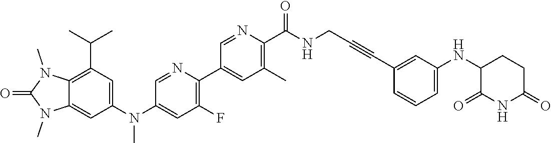 [SMILES: Cc1cc(-c2ncc(N(C)c3cc(C(C)C)c4c(c3)n(C)c(=O)n4C)cc2F)cnc1C(=O)NCC#Cc1cccc(NC2CCC(=O)NC2=O)c1] | N-(3-(3-((2,6- dioxopiperidin-3-yl) amino)phenyl)prop-2-yn- 1-yl)-3-fluoro-5-((7- isopropyl-1,3-dimethyl-2- oxo-2,3-dihydro-1H-benzo [d]imidazol-5-yl)(methyl) amino)-5′-methyl-[2,3′- bipyridine]-6′-carboxamide |
| A48 | 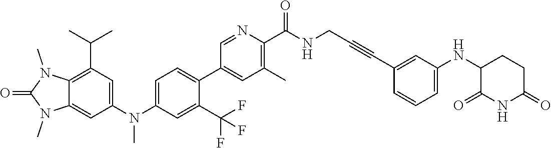 [SMILES: Cc1cc(-c2ccc(N(C)c3cc(C(C)C)c4c(c3)n(C)c(=O)n4C)cc2C(F)(F)F)cnc1C(=O)NCC#Cc1cccc(NC2CCC(=O)NC2=O)c1] | N-(3-(3-((2,6- dioxopiperidin-3-yl) amino)phenyl)prop-2-yn- 1-yl)-5-(4-((7-isopropyl- 1,3-dimethyl-2-oxo-2,3- dihydro-1H-benzo [d]imidazol-5- yl)(methyl)amino)-2- (trifluoromethyl)phenyl)-3- methylpicolinamide |
| A49 | 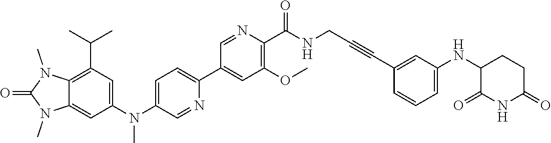 [SMILES: COc1cc(-c2ccc(N(C)c3cc(C(C)C)c4c(c3)n(C)c(=O)n4C)cn2)cnc1C(=O)NCC#Cc1cccc(NC2CCC(=O)NC2=O)c1] | N-(3-(3-((2,6- dioxopiperidin-3-yl) amino)phenyl)prop-2-yn- 1-yl)-5-((7-isopropyl-1,3- dimethyl-2-oxo-2,3- dihydro-1H-benzo [d]imidazol-5-yl) (methyl)amino)-5′- methoxy-[2,3′- |
| | | bipyridine]-6′- |
| | | carboxamide |
| A50 | 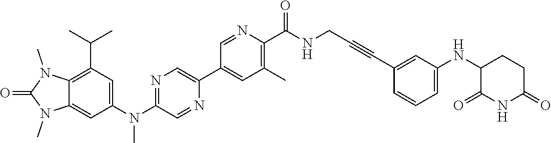 [SMILES: Cc1cc(-c2cnc(N(C)c3cc(C(C)C)c4c(c3)n(C)c(=O)n4C)cn2)cnc1C(=O)NCC#Cc1cccc(NC2CCC(=O)NC2=O)c1] | N-(3-(3-((2,6- dioxopiperidin-3-yl) amino)phenyl)prop-2-yn- 1-yl)-5-(5-((7-isopropyl- 1,3-dimethyl-2-oxo-2,3- dihydro-1H-benzo [d]imidazol-5- yl)(methyl)amino) pyrazin-2-yl)-3- methylpicolinamide |
| A51 | 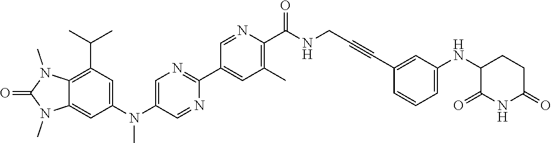 [SMILES: Cc1cc(-c2ncc(N(C)c3cc(C(C)C)c4c(c3)n(C)c(=O)n4C)cn2)cnc1C(=O)NCC#Cc1cccc(NC2CCC(=O)NC2=O)c1] | N-(3-(3-((2,6- dioxopiperidin-3-yl) amino)phenyl)prop-2-yn- 1-yl)-5-(5-((7-isopropyl- 1,3-dimethyl-2-oxo-2,3- dihydro-1H-benzo [d]imidazol-5-yl)(methyl) amino)pyrimidin-2- yl)-3-methylpicolinamide |
| A52 | 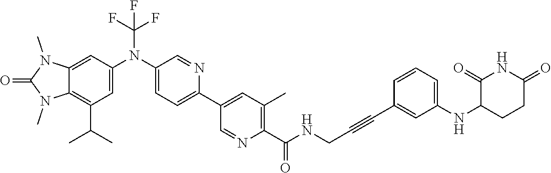 [SMILES: Cc1cc(-c2ccc(N(c3cc(C(C)C)c4c(c3)n(C)c(=O)n4C)C(F)(F)F)cn2)cnc1C(=O)NCC#Cc1cccc(NC2CCC(=O)NC2=O)c1] | N-(3-(3-((2,6- dioxopiperidin-3- yl)amino)phenyl)prop-2- yn-1-yl)-5-((7-isopropyl- 1,3-dimethyl-2-oxo-2,3- dihydro-1H-benzo [d]imidazol-5-yl) (trifluoromethyl)amino)-5′- methyl-[2,3′-bipyridine]- 6′-carboxamide |
| A53 | 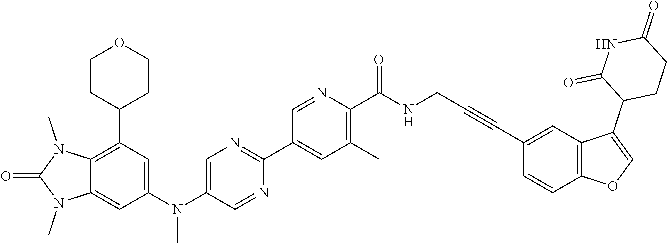 [SMILES: Cc1cc(-c2ncc(N(C)c3cc(C4CCOCC4)c4c(c3)n(C)c(=O)n4C)cn2)cnc1C(=O)NCC#Cc1ccc2occ(C3CCC(=O)NC3=O)c2c1] | 5-(5-((1,3-dimethyl-2- oxo-7-(tetrahydro-2H- pyran-4-yl)-2,3- dihydro-1H- benzo[d]imidazol-5- yl)(methyl)amino) pyrimidin-2-yl)-N- (3-(3-(2,6-dioxo- piperidin-3-yl) benzofuran-5- yl)prop-2-yn-1-yl)-3- methylpicolinamide |
| A54 | 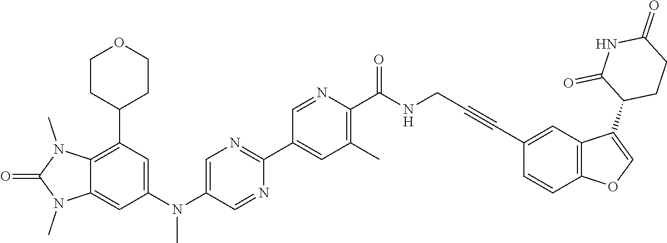 [SMILES: Cc1cc(-c2ncc(N(C)c3cc(C4CCOCC4)c4c(c3)n(C)c(=O)n4C)cn2)cnc1C(=O)NCC#Cc1ccc2occ([C@H]3CCC(=O)NC3=O)c2c1] | (R)-5-(5-((1,3-dimethyl- 2-oxo-7-(tetrahydro- 2H-pyran-4-yl)- 2,3-dihydro- 1H-benzo[d]imidazol-5- yl)(methyl)amino) pyrimidin-2-yl)-N-(3- (3-(2,6-dioxopiperidin- 3-yl)benzofuran-5- yl)prop-2-yn-1-yl)- 3-methylpicolinamide |
| A55 | 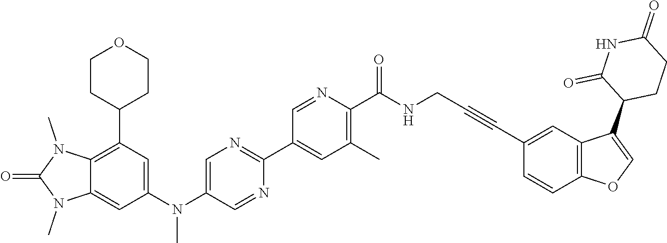 [SMILES: Cc1cc(-c2ncc(N(C)c3cc(C4CCOCC4)c4c(c3)n(C)c(=O)n4C)cn2)cnc1C(=O)NCC#Cc1ccc2occ([C@@H]3CCC(=O)NC3=O)c2c1] | (S)-5-(5-((1,3-dimethyl- 2-oxo-7-(tetrahydro- 2H-pyran-4-yl)- 2,3-dihydro- 1H-benzo[d]imidazol-5- yl)(methyl)amino) pyrimidin- 2-yl)-N-(3-(3-(2,6-dioxo- piperidin-3-yl) benzofuran-5- yl)prop-2-yn-1-yl)- 3-methylpicolinamide |
| A56 | 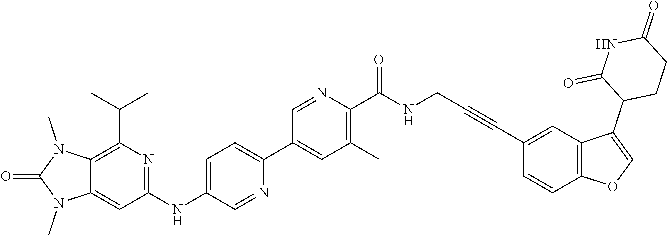 [SMILES: Cc1cc(-c2ccc(Nc3cc4c(c(C(C)C)n3)n(C)c(=O)n4C)cn2)cnc1C(=O)NCC#Cc1ccc2occ(C3CCC(=O)NC3=O)c2c1] | N-(3-(3-(2,6- dioxopiperidin-3- yl)benzofuran-5-yl)prop- 2-yn-1-yl)-5-((4-isopropyl- 1,3-dimethyl-2-oxo-2,3- dihydro-1H-imidazo[4,5- c]pyridin-6-yl)amino)-5′- methyl-[2,3′-bipyridine]- 6′-carboxamide |
| A57 | 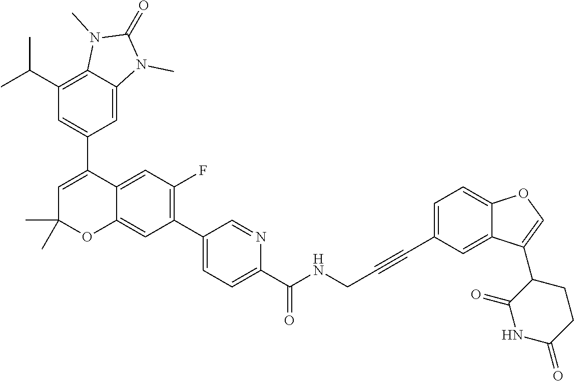 [SMILES: CC(C)c1cc(C2=CC(C)(C)Oc3cc(-c4ccc(C(=O)NCC#Cc5ccc6occ(C7CCC(=O)NC7=O)c6c5)nc4)c(F)cc32)cc2c1n(C)c(=O)n2C] | N-(3-(3-(2,6- dioxopiperidin-3- yl)benzofuran-5-yl)prop- 2-yn-1-yl)-5-(6-fluoro-4- (7-isopropyl-1,3-dimethyl- 2-oxo-2,3-dihydro-1H- benzo[d]imidazol-5-yl)- 2,2-dimethyl- 2H-chromen-7- yl)picolinamide |
| A58 | 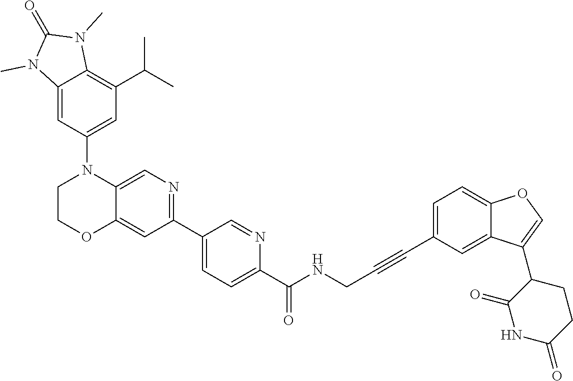 [SMILES: CC(C)c1cc(N2CCOc3cc(-c4ccc(C(=O)NCC#Cc5ccc6occ(C7CCC(=O)NC7=O)c6c5)nc4)ncc32)cc2c1n(C)c(=O)n2C] | N-(3-(3-(2,6- dioxopiperidin-3- yl)benzofuran-5-yl)prop- 2-yn-1-yl)-5- (4-(7-isopropyl- 1,3-dimethyl-2-oxo-2,3- dihydro-1H-benzo[d] imidazol-5-yl)-3,4- dihydro-2H-pyrido[4,3- b][1,4]oxazin-7- yl)picolinamide |
| A59 | 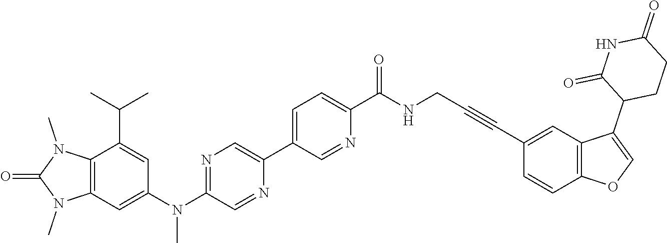 [SMILES: CC(C)c1cc(N(C)c2cnc(-c3ccc(C(=O)NCC#Cc4ccc5occ(C6CCC(=O)NC6=O)c5c4)nc3)cn2)cc2c1n(C)c(=O)n2C] | N-(3-(3-(2,6- dioxopiperidin-3- yl)benzofuran-5-yl)prop- 2-yn-1-yl)-5- (5-((7-isopropyl- 1,3-dimethyl-2-oxo-2,3- dihydro-1H-benzo [d]imidazol-5- yl)(methyl)amino) pyrazin-2- yl)picolinamide |
| A60 | 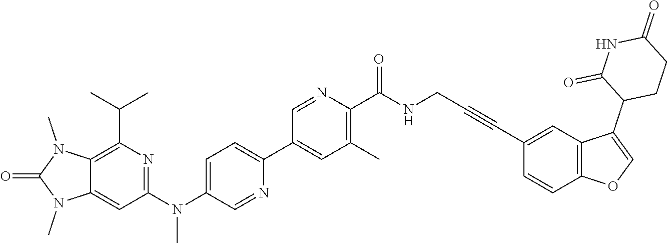 [SMILES: Cc1cc(-c2ccc(N(C)c3cc4c(c(C(C)C)n3)n(C)c(=O)n4C)cn2)cnc1C(=O)NCC#Cc1ccc2occ(C3CCC(=O)NC3=O)c2c1] | N-(3-(3-(2,6- dioxopiperidin-3- yl)benzofuran-5-yl)prop- 2-yn-1-yl)-5-((4-isopropyl- 1,3-dimethyl-2-oxo-2,3- dihydro-1H-imidazo[4,5- c]pyridin-6-yl)(methyl) amino)-5′-methyl-[2,3′- bipyridine]-6′-carboxamide |
| A61 | 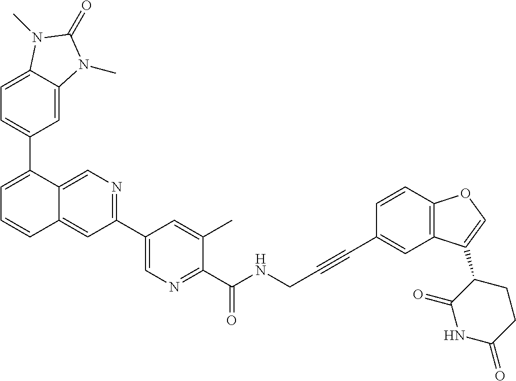 [SMILES: Cc1cc(-c2cc3cccc(-c4ccc5c(c4)n(C)c(=O)n5C)c3cn2)cnc1C(=O)NCC#Cc1ccc2occ([C@@H]3CCC(=O)NC3=O)c2c1] | (S)-5-(8-(1,3-dimethyl- 2-oxo-2,3-dihydro- 1H-benzo[d]imidazol- 5-yl)isoquinolin-3- yl)-N-(3-(3-(2,6-dioxo- piperidin-3-yl)benzofuran- 5-yl)prop-2-yn-1-yl)-3- methylpicolinamide |
| A62 | 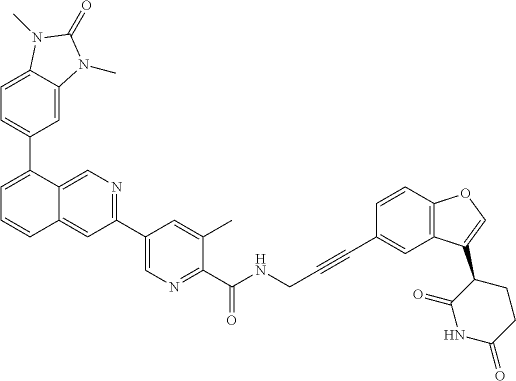 [SMILES: Cc1cc(-c2cc3cccc(-c4ccc5c(c4)n(C)c(=O)n5C)c3cn2)cnc1C(=O)NCC#Cc1ccc2occ([C@H]3CCC(=O)NC3=O)c2c1] | (R)-5-(8-(1,3-dimethyl- 2-oxo-2,3-dihydro-1H- benzo[d]imidazol-5- yl)isoquinolin-3-yl)- N-(3-(3-(2,6-dioxo- piperidin-3-yl)benzofuran- 5-yl)prop-2-yn-1-yl)-3- methylpicolinamide |
| A63 | 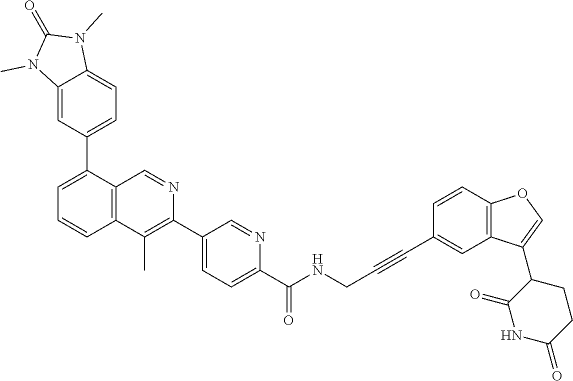 [SMILES: Cc1c(-c2ccc(C(=O)NCC#Cc3ccc4occ(C5CCC(=O)NC5=O)c4c3)nc2)ncc2c(-c3ccc4c(c3)n(C)c(=O)n4C)cccc12] | 5-(8-(1,3-dimethyl-2- oxo-2,3-dihydro-1H- benzo[d]imidazol-5-yl)-4- methylisoquinolin-3- yl)-N-(3-(3-(2,6-dioxo- piperidin-3-yl) benzofuran-5- yl)prop-2-yn-1- yl)picolinamide |
| A64 | 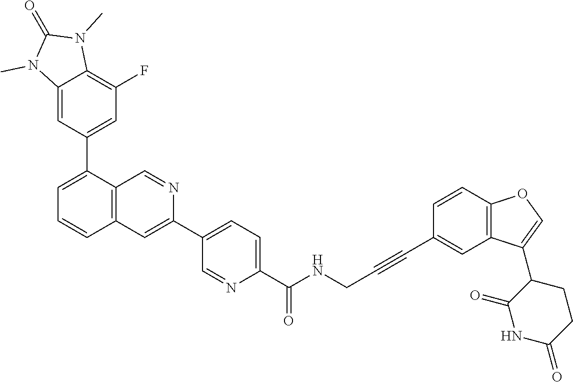 [SMILES: Cn1c(=O)n(C)c2c(F)cc(-c3cccc4cc(-c5ccc(C(=O)NCC#Cc6ccc7occ(C8CCC(=O)NC8=O)c7c6)nc5)ncc34)cc21] | N-(3-(3-(2,6- dioxopiperidin-3- yl)benzofuran-5-yl)prop- 2-yn-1-yl)-5-(8- (7-fluoro-1,3-dimethyl- 2-oxo-2,3-dihydro-1H- benzo[d]imidazol-5- yl)isoquinolin-3- yl)picolinamide |
| A65 | 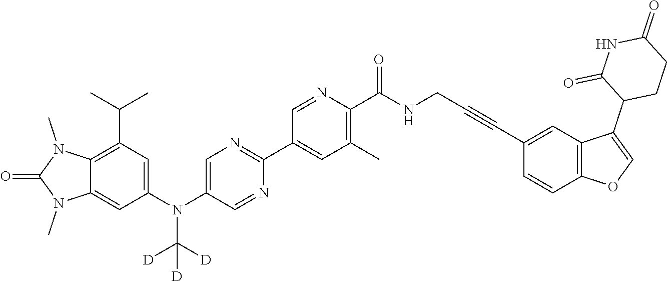 [SMILES: [2H]C([2H])([2H])N(c1cnc(-c2cnc(C(=O)NCC#Cc3ccc4occ(C5CCC(=O)NC5=O)c4c3)c(C)c2)nc1)c1cc(C(C)C)c2c(c1)n(C)c(=O)n2C] | N-(3-(3-(2,6- dioxopiperidin-3- yl)benzofuran-5-yl)prop- 2-yn-1-yl)-5-(5- ((7-isopropyl- 1,3-dimethyl-2-oxo-2,3- dihydro-1H-benzo [d]imidazol- 5-yl)(methyl-d3)amino) pyrimidin-2-yl)-3- methylpicolinamide |
| A66 | 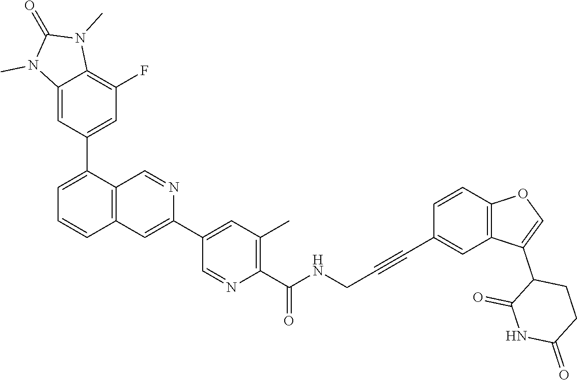 [SMILES: Cc1cc(-c2cc3cccc(-c4cc(F)c5c(c4)n(C)c(=O)n5C)c3cn2)cnc1C(=O)NCC#Cc1ccc2occ(C3CCC(=O)NC3=O)c2c1] | N-(3-(3-(2,6- dioxopiperidin-3- yl)benzofuran-5-yl)prop- 2-yn-1-yl)-5-(8- (7-fluoro-1,3-dimethyl- 2-oxo-2,3-dihydro-1H- benzo[d]imidazol-5- yl)isoquinolin-3-yl)-3- methylpicolinamide |
| A67 | 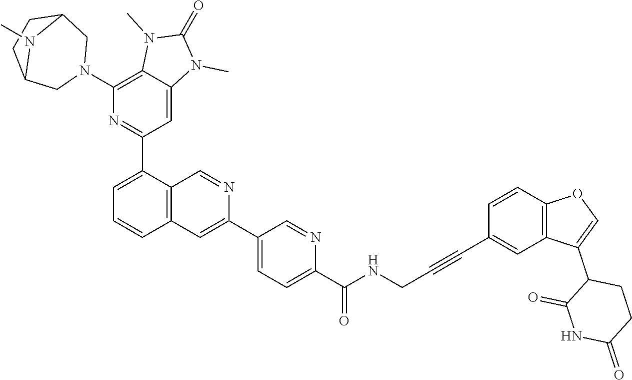 [SMILES: CN1C2CCC1CN(c1nc(-c3cccc4cc(-c5ccc(C(=O)NCC#Cc6ccc7occ(C8CCC(=O)NC8=O)c7c6)nc5)ncc34)cc3c1n(C)c(=O)n3C)C2] | 5-(8-(1,3-dimethyl-4- (8-methyl-3,8- diazabicyclo[3.2.1] octan-3-yl)-2-oxo-2,3- dihydro-1H-imidazo [4,5-c]pyridin-6- yl)isoquinolin-3- yl)-N-(3-(3-(2,6- dioxopiperidin-3- yl)benzofuran-5- yl)prop-2-yn-1- yl)picolinamide |
| A68 | 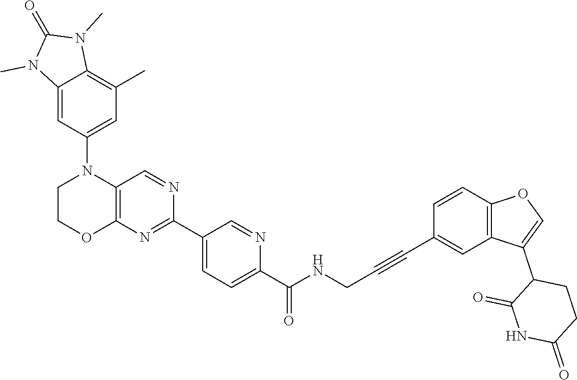 [SMILES: Cc1cc(N2CCOc3nc(-c4ccc(C(=O)NCC#Cc5ccc6occ(C7CCC(=O)NC7=O)c6c5)nc4)ncc32)cc2c1n(C)c(=O)n2C] | N-(3-(3-(2,6- dioxopiperidin-3- yl)benzofuran-5-yl)prop-2- yn-1-yl)-5-(5- (1,3,7-trimethyl- 2-oxo-2,3-dihydro-1H- benzo[d]imidazol-5-yl)- 6,7-dihydro-5H-pyrimido [4,5-b][1,4]oxazin-2- yl)picolinamide |
| A69 | 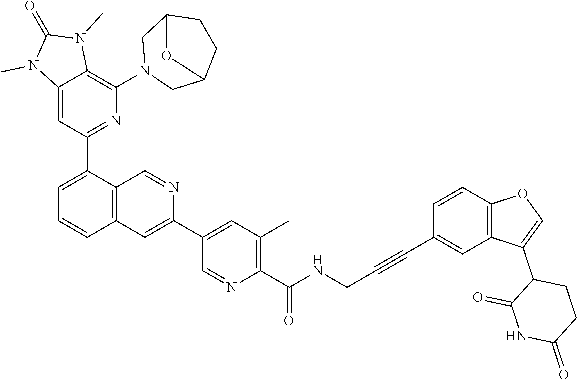 [SMILES: Cc1cc(-c2cc3cccc(-c4cc5c(c(N6CC7CCC(C6)O7)n4)n(C)c(=O)n5C)c3cn2)cnc1C(=O)NCC#Cc1ccc2occ(C3CCC(=O)NC3=O)c2c1] | 5-(8-(4-(8-oxa- 3-azabicyclo [3.2.1]octan-3-yl)-1,3- dimethyl-2-oxo- 2,3-dihydro- 1H-imidazo[4,5-c] pyridin-6-yl)isoquinolin-3- yl)-N-(3-(3-(2,6-dioxo- piperidin-3-yl)benzofuran- 5-yl)prop-2-yn-1-yl)-3- methylpicolinamide |
| A70 | 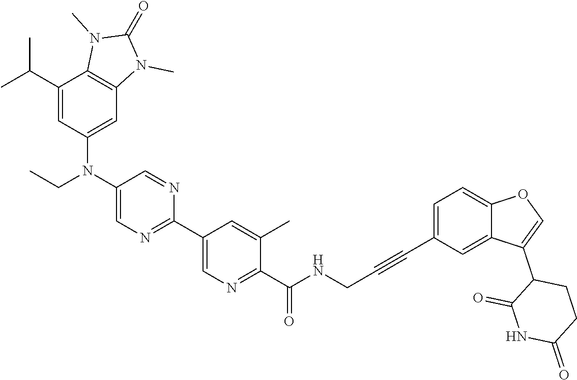 [SMILES: CCN(c1cnc(-c2cnc(C(=O)NCC#Cc3ccc4occ(C5CCC(=O)NC5=O)c4c3)c(C)c2)nc1)c1cc(C(C)C)c2c(c1)n(C)c(=O)n2C] | N-(3-(3-(2,6- dioxopiperidin-3- yl)benzofuran-5-yl)prop- 2-yn-1-yl)-5-(5-(ethyl(7- isopropyl-1,3- dimethyl-2-oxo- 2,3-dihydro-1H-benzo[d] imidazol-5-yl)amino) pyrimidin-2-yl)-3- methylpicolinamide |
| A71 | 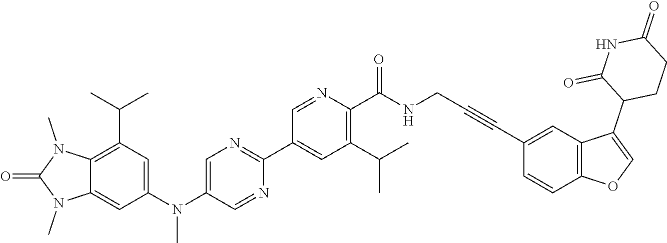 [SMILES: CC(C)c1cc(-c2ncc(N(C)c3cc(C(C)C)c4c(c3)n(C)c(=O)n4C)cn2)cnc1C(=O)NCC#Cc1ccc2occ(C3CCC(=O)NC3=O)c2c1] | N-(3-(3-(2,6- dioxopiperidin-3- yl)benzofuran-5-yl)prop- 2-yn-1-yl)-3-isopropyl-5- (5-((7-isopropyl-1,3- dimethyl-2-oxo-2,3- dihydro-1H- benzo[d]imidazol-5- yl)(methyl)amino) pyrimidin-2- yl)picolinamide |
| A72 | 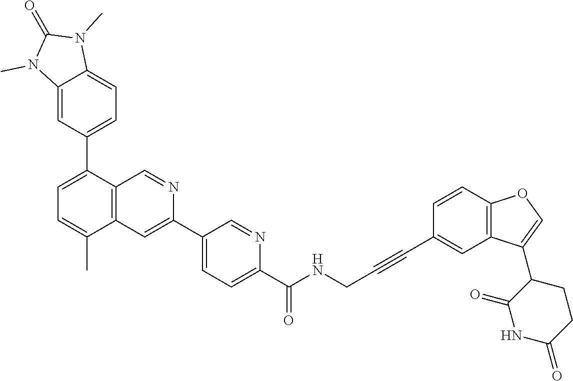 [SMILES: Cc1ccc(-c2ccc3c(c2)n(C)c(=O)n3C)c2cnc(-c3ccc(C(=O)NCC#Cc4ccc5occ(C6CCC(=O)NC6=O)c5c4)nc3)cc12] | 5-(8-(1,3-dimethyl-2- oxo-2,3-dihydro-1H- benzo[d]imidazol-5-yl)-5- methylisoquinolin-3-yl)- N-(3-(3-(2,6- dioxopiperidin- 3-yl)benzofuran-5- yl)prop-2-yn-1- yl)picolinamide |
| A73 | 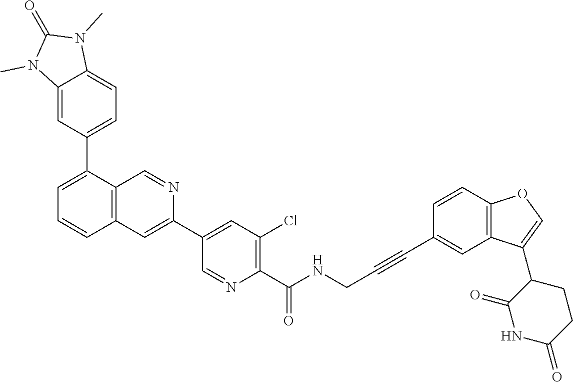 [SMILES: Cn1c(=O)n(C)c2cc(-c3cccc4cc(-c5cnc(C(=O)NCC#Cc6ccc7occ(C8CCC(=O)NC8=O)c7c6)c(Cl)c5)ncc34)ccc21] | 3-chloro-5- (8-(1,3-dimethyl- 2-oxo-2,3-dihydro- 1H-benzo[d]imidazol-5- yl)isoquinolin-3- yl)-N-(3-(3-(2,6- dioxopiperidin-3- yl)benzofuran-5- yl)prop-2-yn-1- yl)picolinamide |
| A74 | 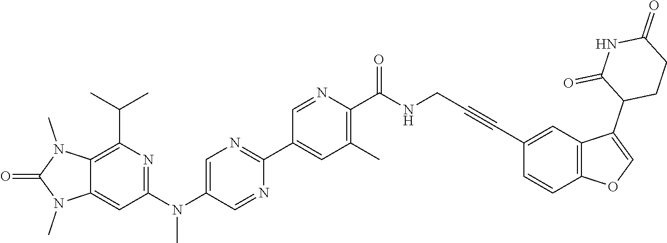 [SMILES: Cc1cc(-c2ncc(N(C)c3cc4c(c(C(C)C)n3)n(C)c(=O)n4C)cn2)cnc1C(=O)NCC#Cc1ccc2occ(C3CCC(=O)NC3=O)c2c1] | N-(3-(3-(2,6- dioxopiperidin-3- yl)benzofuran-5-yl)prop-2- yn-1-yl)-5-(5-((4-isopropyl- 1,3-dimethyl-2-oxo-2,3- dihydro-1H-imidazo[4,5- c]pyridin-6-yl)(methyl) amino)pyrimidin-2-yl)-3- methylpicolinamide |
| A75 | 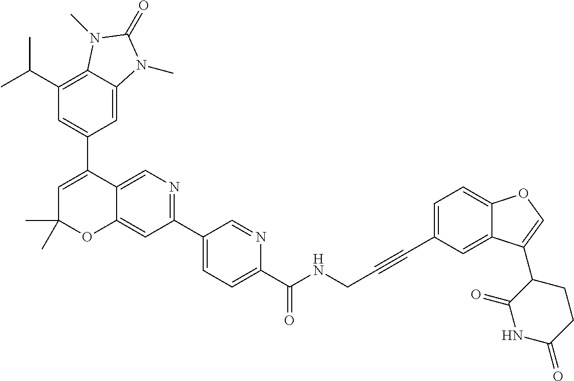 [SMILES: CC(C)c1cc(C2=CC(C)(C)Oc3cc(-c4ccc(C(=O)NCC#Cc5ccc6occ(C7CCC(=O)NC7=O)c6c5)nc4)ncc32)cc2c1n(C)c(=O)n2C] | N-(3-(3-(2,6- dioxopiperidin-3- yl)benzofuran-5-yl)prop-2- yn-1-yl)-5-(4-(7-isopropyl- 1,3-dimethyl-2-oxo-2,3- dihydro-1H-benzo[d] imidazol-5-yl)- 2,2-dimethyl-2H- pyrano[3,2-c]pyridin-7- yl)picolinamide |
| A76 | 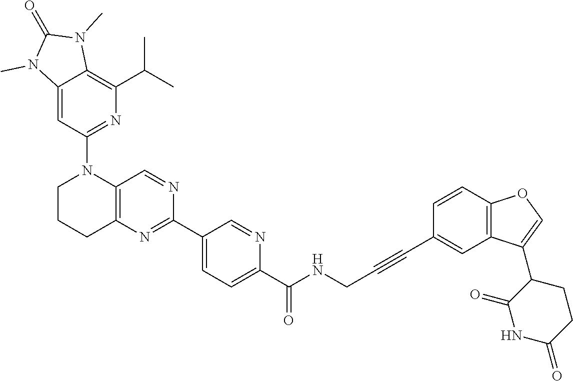 [SMILES: CC(C)c1nc(N2CCCc3nc(-c4ccc(C(=O)NCC#Cc5ccc6occ(C7CCC(=O)NC7=O)c6c5)nc4)ncc32)cc2c1n(C)c(=O)n2C] | N-(3-(3-(2,6- dioxopiperidin-3- yl)benzofuran-5- yl)prop-2-yn-1-yl)-5- (5-(4-isopropyl- 1,3-dimethyl-2-oxo-2,3- dihydro-1H-imidazo[4,5- c]pyridin-6-yl)-5,6,7,8- tetrahydropyrido[3,2- d]pyrimidin- 2-yl)picolinamide |
| A77 | 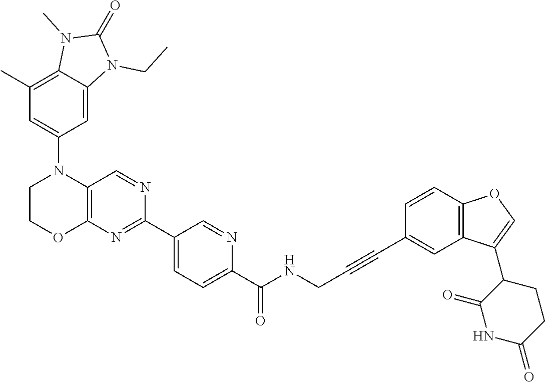 [SMILES: CCn1c(=O)n(C)c2c(C)cc(N3CCOc4nc(-c5ccc(C(=O)NCC#Cc6ccc7occ(C8CCC(=O)NC8=O)c7c6)nc5)ncc43)cc21] | N-(3-(3-(2,6- dioxopiperidin-3- yl)benzofuran-5-yl)prop- 2-yn-1-yl)-5-(5-(3-ethyl- 1,7-dimethyl-2-oxo-2,3- dihydro-1H-benzo[d] imidazol-5-yl)- 6,7-dihydro- 5H-pyrimido[4,5-b][1,4] oxazin-2-yl)picolinamide |
| A78 | 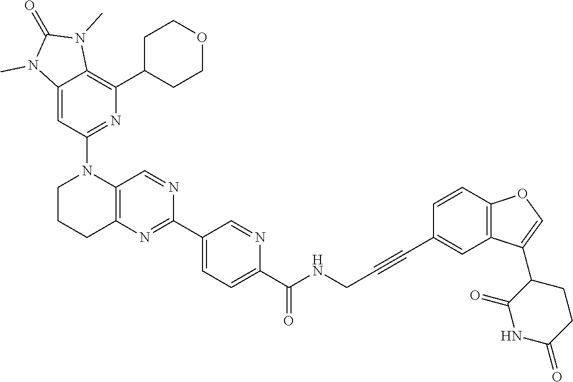 [SMILES: Cn1c(=O)n(C)c2c(C3CCOCC3)nc(N3CCCc4nc(-c5ccc(C(=O)NCC#Cc6ccc7occ(C8CCC(=O)NC8=O)c7c6)nc5)ncc43)cc21] | 5-(5-(1,3-dimethyl-2- oxo-4-(tetrahydro- 2H-pyran- 4-yl)-2,3-dihydro-1H- imidazo[4,5-c]pyridin- 6-yl)-5,6,7,8-tetrahydro- pyrido[3,2-d]pyrimidin- 2-yl)-N-(3-(3-(2,6- dioxopiperidin-3- yl)benzofuran-5-yl)prop- 2-yn-1- yl)picolinamide |
| A79 | 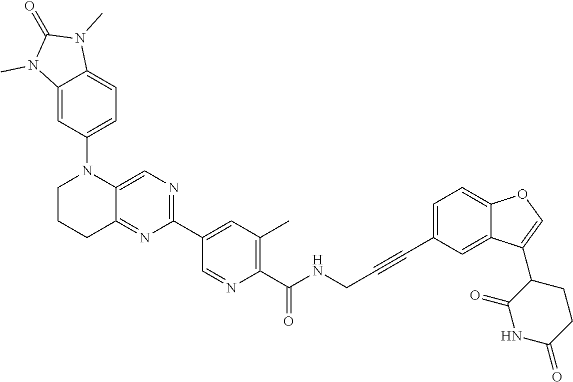 [SMILES: Cc1cc(-c2ncc3c(n2)CCCN3c2ccc3c(c2)n(C)c(=O)n3C)cnc1C(=O)NCC#Cc1ccc2occ(C3CCC(=O)NC3=O)c2c1] | 5-(5-(1,3-dimethyl-2- oxo-2,3-dihydro-1H- benzo[d]imidazol-5- yl)-5,6,7,8- tetrahydropyrido[3,2- d]pyrimidin-2-yl)- N-(3-(3-(2,6-dioxo- piperidin-3- yl)benzofuran-5-yl)prop- 2-yn-1-yl)-3- methylpicolinamide |
| A80 | 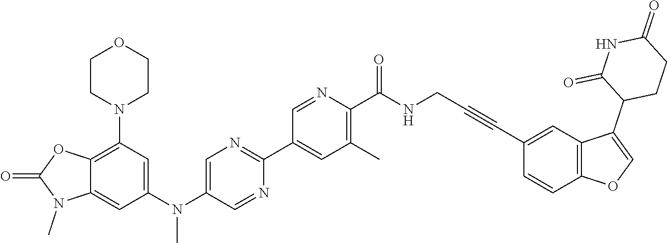 [SMILES: Cc1cc(-c2ncc(N(C)c3cc(N4CCOCC4)c4oc(=O)n(C)c4c3)cn2)cnc1C(=O)NCC#Cc1ccc2occ(C3CCC(=O)NC3=O)c2c1] | N-(3-(3-(2,6-dioxo- piperidin-3- yl)benzofuran-5-yl) prop-2-yn-1-yl)-3- methyl-5-(5-(methyl (3-methyl-7- morpholino-2-oxo- 2,3-dihydrobenzo [d]oxazol-5- yl)amino)pyrimidin- 2-yl)picolinamide |
| A81 | 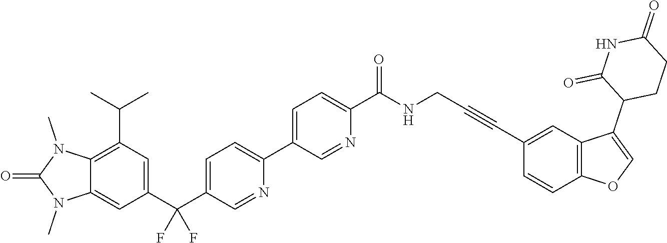 [SMILES: CC(C)c1cc(C(F)(F)c2ccc(-c3ccc(C(=O)NCC#Cc4ccc5occ(C6CCC(=O)NC6=O)c5c4)nc3)nc2)cc2c1n(C)c(=O)n2C] | 5-(difluoro(7-isopropyl- 1,3-dimethyl-2- oxo-2,3-dihydro-1H- benzo[d]imidazol-5- yl)methyl)-N-(3-(3- (2,6-dioxopiperidin-3- yl)benzofuran-5-yl) prop-2-yn-1-yl)-[2,3′- bipyridine]-6′- carboxamide |
| A82 | 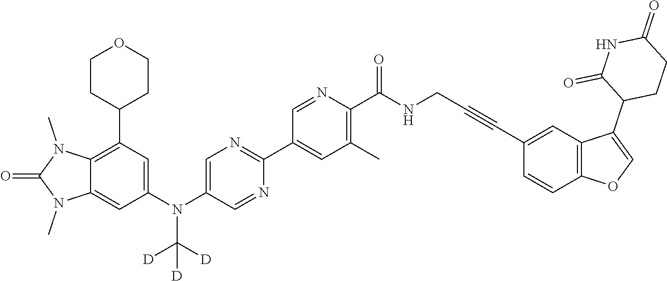 [SMILES: [2H]C([2H])([2H])N(c1cnc(-c2cnc(C(=O)NCC#Cc3ccc4occ(C5CCC(=O)NC5=O)c4c3)c(C)c2)nc1)c1cc(C2CCOCC2)c2c(c1)n(C)c(=O)n2C] | 5-(5-((1,3-dimethyl- 2-oxo-7-(tetrahydro- 2H-pyran-4-yl)-2,3- dihydro-1H-benzo [d]imidazol-5-yl) (methyl-d3)amino) pyrimidin-2-yl)-N-(3- (3-(2,6-dioxo- piperidin-3-yl) benzofuran-5- yl)prop-2-yn-1-yl)- 3-methylpicolinamide |
| A83 | 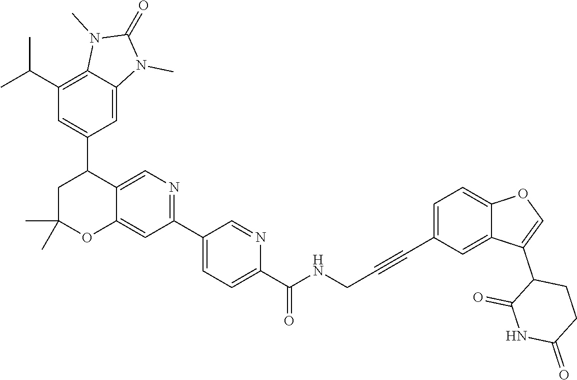 [SMILES: CC(C)c1cc(C2CC(C)(C)Oc3cc(-c4ccc(C(=O)NCC#Cc5ccc6occ(C7CCC(=O)NC7=O)c6c5)nc4)ncc32)cc2c1n(C)c(=O)n2C] | N-(3-(3-(2,6-dioxo- piperidin-3- yl)benzofuran-5-yl) prop-2-yn-1-yl)-5-(4- (7-isopropyl-1,3- dimethyl-2-oxo-2,3- dihydro-1H-benzo [d]imidazol-5-yl)-2,2- dimethyl-3,4-dihydro- 2H-pyrano[3,2- c]pyridin-7-yl) picolinamide |
| A84 | 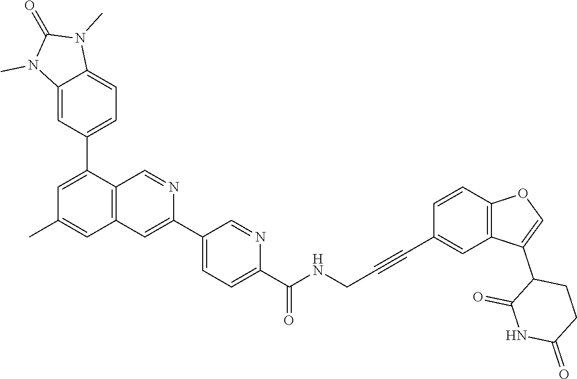 [SMILES: Cc1cc(-c2ccc3c(c2)n(C)c(=O)n3C)c2cnc(-c3ccc(C(=O)NCC#Cc4ccc5occ(C6CCC(=O)NC6=O)c5c4)nc3)cc2c1] | 5-(8-(1,3-dimethyl-2- oxo-2,3-dihydro-1H- benzo[d]imidazol-5- yl)-6- methylisoquinolin- 3-yl)-N- (3-(3-(2,6-dioxo- piperidin-3-yl) benzofuran-5- yl)prop-2-yn-1-yl) picolinamide |
| A85 | 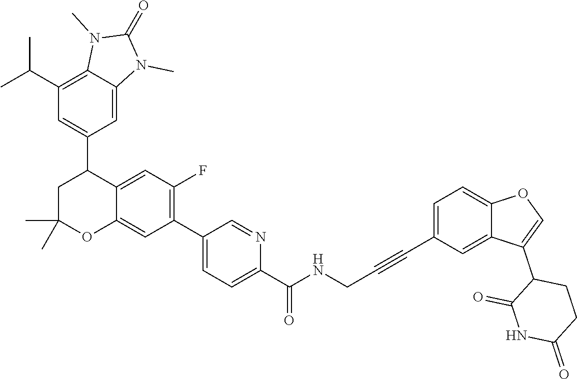 [SMILES: CC(C)c1cc(C2CC(C)(C)Oc3cc(-c4ccc(C(=O)NCC#Cc5ccc6occ(C7CCC(=O)NC7=O)c6c5)nc4)c(F)cc32)cc2c1n(C)c(=O)n2C] | N-(3-(3-(2,6-dioxo- piperidin-3- yl)benzofuran-5-yl) prop-2-yn-1-yl)-5-(6- fluoro-4-(7-isopropyl- 1,3-dimethyl-2-oxo- 2,3-dihydro-1H-benzo [d]imidazol-5-yl)- 2,2-dimethylchroman- 7-yl)picolinamide |
| A86 | 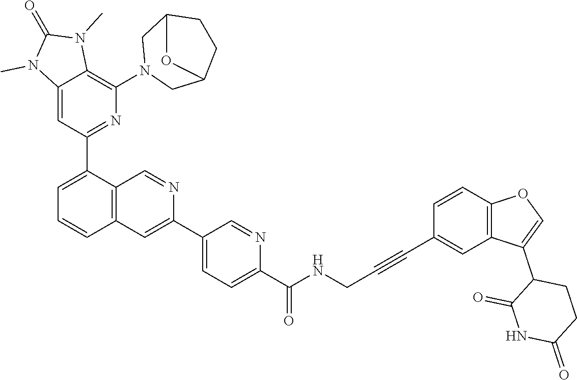 [SMILES: Cn1c(=O)n(C)c2c(N3CC4CCC(C3)O4)nc(-c3cccc4cc(-c5ccc(C(=O)NCC#Cc6ccc7occ(C8CCC(=O)NC8=O)c7c6)nc5)ncc34)cc21] | 5-(8-(4-(8-oxa-3- azabicyclo[3.2.1]octan- 3-yl)-1,3-dimethyl-2- oxo-2,3-dihydro-1H- imidazo[4,5-c]pyridin- 6-yl)isoquinolin-3- yl)-N-(3-(3-(2,6-dioxo- piperidin-3- yl)benzofuran-5-yl) prop-2-yn-1- yl)picolinamide |
| A87 | 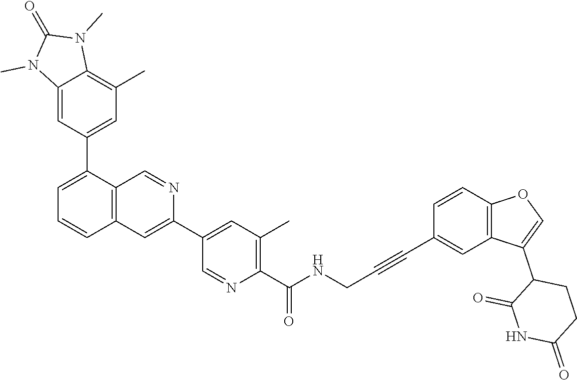 [SMILES: Cc1cc(-c2cc3cccc(-c4cc(C)c5c(c4)n(C)c(=O)n5C)c3cn2)cnc1C(=O)NCC#Cc1ccc2occ(C3CCC(=O)NC3=O)c2c1] | N-(3-(3-(2,6-dioxo- piperidin-3- yl)benzofuran-5-yl) prop-2-yn-1-yl)-3- methyl-5-(8-(1,3,7- trimethyl-2-oxo-2,3- dihydro-1H-benzo [d]imidazol-5- yl)isoquinolin-3- yl)picolinamide |
| A88 | 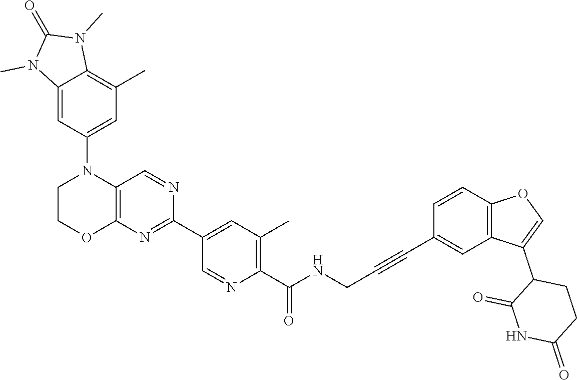 [SMILES: Cc1cc(-c2ncc3c(n2)OCCN3c2cc(C)c3c(c2)n(C)c(=O)n3C)cnc1C(=O)NCC#Cc1ccc2occ(C3CCC(=O)NC3=O)c2c1] | N-(3-(3-(2,6-dioxo- piperidin-3- yl)benzofuran-5-yl) prop-2-yn-1-yl)-3- methyl-5-(5-(1,3,7- trimethyl-2-oxo-2,3- dihydro-1H-benzo [d]imidazol-5-yl)-6,7- dihydro-5H-pyrimido [4,5-b][1,4]oxazin- 2-yl)picolinamide |
| A89 | 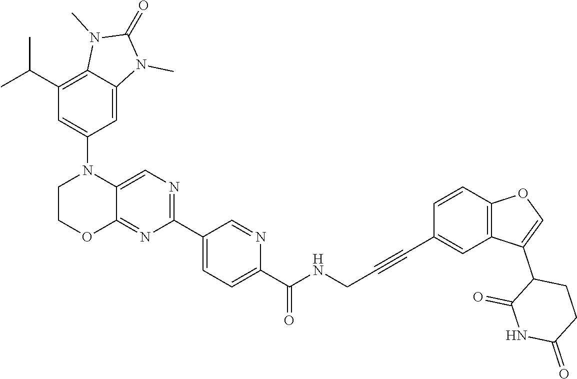 [SMILES: CC(C)c1cc(N2CCOc3nc(-c4ccc(C(=O)NCC#Cc5ccc6occ(C7CCC(=O)NC7=O)c6c5)nc4)ncc32)cc2c1n(C)c(=O)n2C] | N-(3-(3-(2,6-dioxo- piperidin-3- yl)benzofuran-5-yl) prop-2-yn-1-yl)-5-(5- (7-isopropyl-1,3- dimethyl-2-oxo-2,3- dihydro-1H-benzo[d] imidazol-5-yl)-6,7- dihydro-5H-pyrimido [4,5-b][1,4]oxazin- 2-yl)picolinamide |
| A90 | 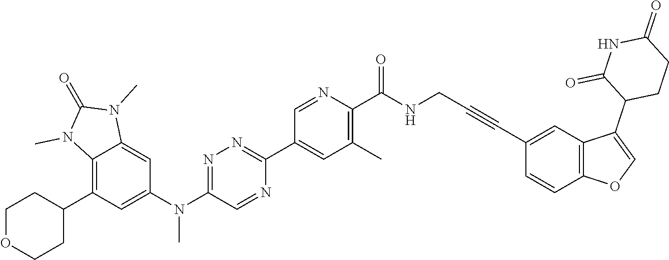 [SMILES: Cc1cc(-c2ncc(N(C)c3cc(C4CCOCC4)c4c(c3)n(C)c(=O)n4C)nn2)cnc1C(=O)NCC#Cc1ccc2occ(C3CCC(=O)NC3=O)c2c1] | 5-(6-((1,3-dimethyl- 2-oxo-7-(tetrahydro- 2H-pyran-4-yl)- 2,3-dihydro-1H- benzo[d]imidazol- 5-yl)(methyl)amino)- 1,2,4-triazin-3-yl)- N-(3-(3-(2,6- dioxopiperidin-3- yl)benzofuran-5- yl)prop-2-yn-1-yl)- 3-methylpicolinamide |
| A91 | 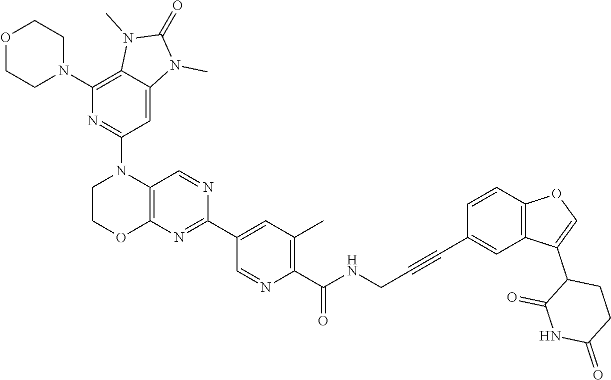 [SMILES: Cc1cc(-c2ncc3c(n2)OCCN3c2cc3c(c(N4CCOCC4)n2)n(C)c(=O)n3C)cnc1C(=O)NCC#Cc1ccc2occ(C3CCC(=O)NC3=O)c2c1] | 5-(5-(1,3-dimethyl- 4-morpholino-2-oxo- 2,3-dihydro-1H- imidazo[4,5-c]pyridin- 6-yl)-6,7-dihydro- 5H-pyrimido[4,5- b][1,4]oxazin-2- yl)-N-(3-(3-(2,6- dioxopiperidin-3- yl)benzofuran-5- yl)prop-2-yn-1-yl)- 3-methylpicolinamide |
| A92 | 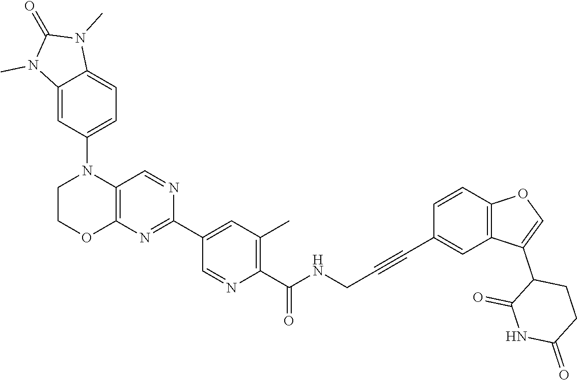 [SMILES: Cc1cc(-c2ncc3c(n2)OCCN3c2ccc3c(c2)n(C)c(=O)n3C)cnc1C(=O)NCC#Cc1ccc2occ(C3CCC(=O)NC3=O)c2c1] | 5-(5-(1,3-dimethyl-2- oxo-2,3-dihydro-1H- benzo[d]imidazol-5- yl)-6,7-dihydro-5H- pyrimido[4,5-b][1,4] oxazin-2-yl)-N-(3-(3- (2,6-dioxopiperidin- 3-yl)benzofuran-5- yl)prop-2-yn-1-yl)- 3-methylpicolinamide |
| A93 | 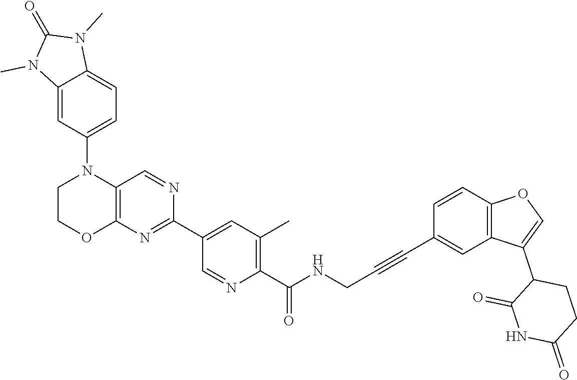 [SMILES: Cc1cc(-c2ncc3c(n2)OCCN3c2ccc3c(c2)n(C)c(=O)n3C)cnc1C(=O)NCC#Cc1ccc2occ(C3CCC(=O)NC3=O)c2c1] | 5-(5-(1,3-dimethyl-2- oxo-2,3-dihydro-1H- benzo[d]imidazol-5- yl)-6,7-dihydro-5H- pyrimido[4,5-b][1,4] oxazin-2-yl)-N-(3-(3- (2,6-dioxopiperidin- 3-yl)benzofuran-5- yl)prop-2-yn-1-yl)- 3-methylpicolinamide |
| A94 | 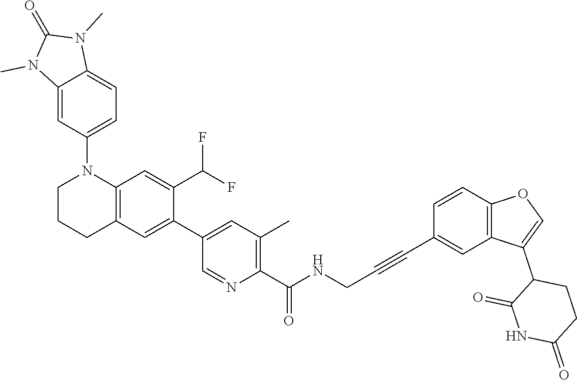 [SMILES: Cc1cc(-c2cc3c(cc2C(F)F)N(c2ccc4c(c2)n(C)c(=O)n4C)CCC3)cnc1C(=O)NCC#Cc1ccc2occ(C3CCC(=O)NC3=O)c2c1] | 5-(7-(difluoromethyl)- 1-(1,3-dimethyl-2- oxo-2,3-dihydro-1H- benzo[d]imidazol-5- yl)-1,2,3,4-tetrahydro- quinolin-6-yl)-N-(3- (3-(2,6-dioxopiperidin- 3-yl)benzofuran-5- yl)prop-2-yn-1-yl)-3- methylpicolinamide |
| A95 | 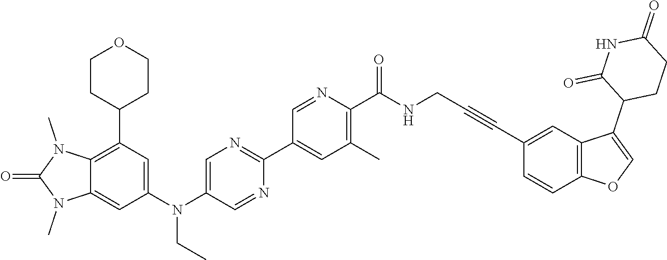 [SMILES: CCN(c1cnc(-c2cnc(C(=O)NCC#Cc3ccc4occ(C5CCC(=O)NC5=O)c4c3)c(C)c2)nc1)c1cc(C2CCOCC2)c2c(c1)n(C)c(=O)n2C] | 5-(5-((1,3-dimethyl- 2-oxo-7-(tetrahydro- 2H-pyran-4-yl)-2,3- dihydro-1H-benzo [d]imidazol-5- yl)(ethyl)amino) pyrimidin-2-yl)-N-(3-(3- (2,6-dioxopiperidin-3- yl)benzofuran-5- yl)prop-2-yn-1-yl)- 3-methylpicolinamide |
| A96 | 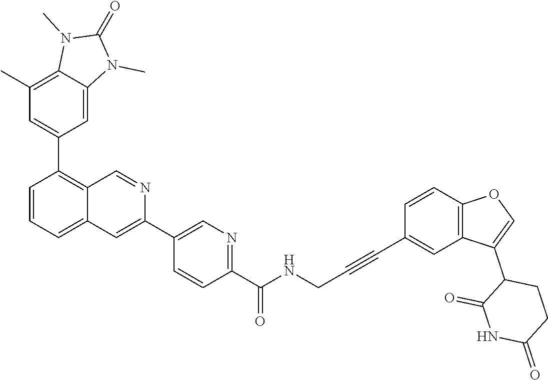 [SMILES: Cc1cc(-c2cccc3cc(-c4ccc(C(=O)NCC#Cc5ccc6occ(C7CCC(=O)NC7=O)c6c5)nc4)ncc23)cc2c1n(C)c(=O)n2C] | N-(3-(3-(2,6-dioxo- piperidin-3- yl)benzofuran-5-yl) prop-2-yn-1-yl)-5-(8- (1,3,7-trimethyl-2- oxo-2,3-dihydro-1H- benzo[d]imidazol-5- yl)isoquinolin-3- yl)picolinamide |
| A97 | 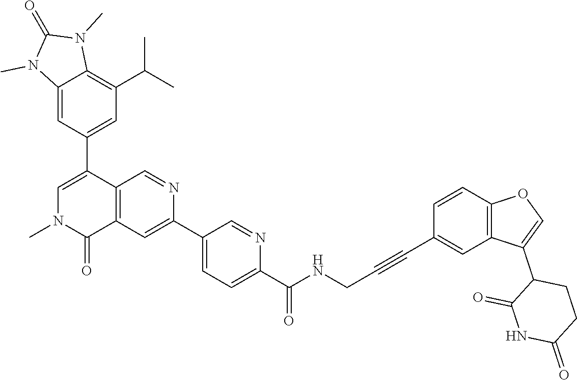 [SMILES: CC(C)c1cc(-c2cn(C)c(=O)c3cc(-c4ccc(C(=O)NCC#Cc5ccc6occ(C7CCC(=O)NC7=O)c6c5)nc4)ncc23)cc2c1n(C)c(=O)n2C] | N-(3-(3-(2,6-dioxo- piperidin-3- yl)benzofuran-5-yl) prop-2-yn-1-yl)-5-(8- (7-isopropyl-1,3- dimethyl-2-oxo-2,3- dihydro-1H-benzo [d]imidazol-5-yl)-6- methyl-5-oxo-5,6- dihydro-2,6- naphthyridin-3-yl) picolinamide |
| A98 | 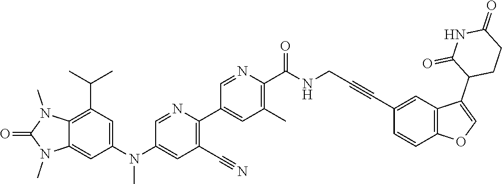 [SMILES: Cc1cc(-c2ncc(N(C)c3cc(C(C)C)c4c(c3)n(C)c(=O)n4C)cc2C#N)cnc1C(=O)NCC#Cc1ccc2occ(C3CCC(=O)NC3=O)c2c1] | 3-cyano-N-(3-(3- (2,6-dioxopiperidin-3- yl)benzofuran-5-yl) prop-2-yn-1-yl)-5-((7- isopropyl-1,3-dimethyl- 2-oxo-2,3-dihydro- 1H-benzo[d]imidazol- 5-yl)(methyl)amino)- 5′-methyl-[2,3′- bipyridine]-6′- carboxamide |
| A99 | 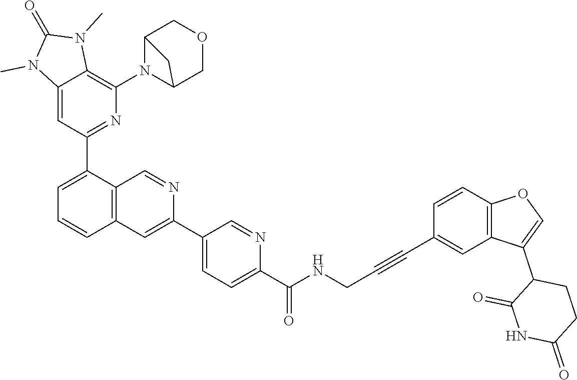 [SMILES: Cn1c(=O)n(C)c2c(N3C4COCC3C4)nc(-c3cccc4cc(-c5ccc(C(=O)NCC#Cc6ccc7occ(C8CCC(=O)NC8=O)c7c6)nc5)ncc34)cc21] | 5-(8-(4-(3-oxa-6- azabicyclo[3.1.1]heptan- 6-yl)-1,3-dimethyl-2- oxo-2,3-dihydro-1H- imidazo[4,5-c]pyridin- 6-yl)isoquinolin-3- yl)-N-(3-(3-(2,6-dioxo- piperidin-3- yl)benzofuran-5-yl) prop-2-yn-1- yl)picolinamide |
| A100 | 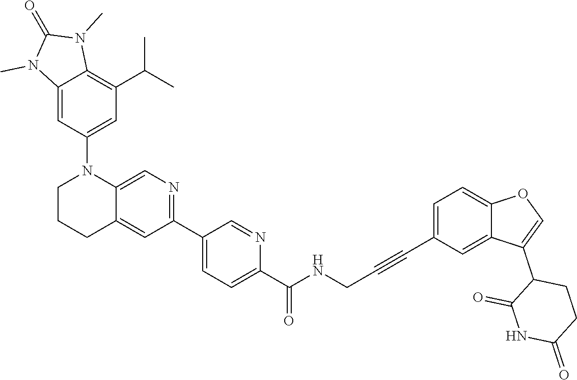 [SMILES: CC(C)c1cc(N2CCCc3cc(-c4ccc(C(=O)NCC#Cc5ccc6occ(C7CCC(=O)NC7=O)c6c5)nc4)ncc32)cc2c1n(C)c(=O)n2C] | N-(3-(3-(2,6-dioxo- piperidin-3- yl)benzofuran-5-yl) prop-2-yn-1-yl)-5-(1- (7-isopropyl-1,3- dimethyl-2-oxo-2,3- dihydro-1H-benzo [d]imidazol-5-yl)- 1,2,3,4-tetrahydro- 1,7-naphthyridin-6- yl)picolinamide |
| A101 | 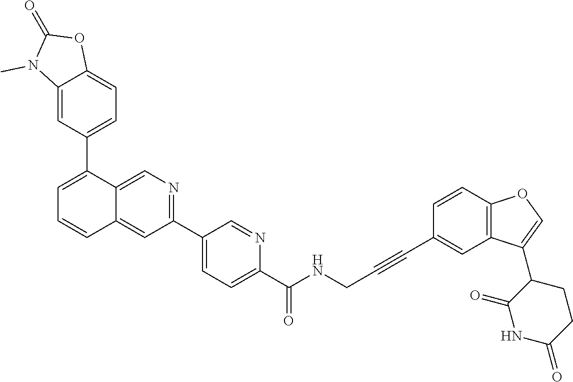 [SMILES: Cn1c(=O)oc2ccc(-c3cccc4cc(-c5ccc(C(=O)NCC#Cc6ccc7occ(C8CCC(=O)NC8=O)c7c6)nc5)ncc34)cc21] | N-(3-(3-(2,6-dioxo- piperidin-3- yl)benzofuran-5-yl) prop-2-yn-1-yl)-5-(8- (3-methyl-2-oxo-2,3- dihydrobenzo[d] oxazol-5-yl)isoquinolin- 3-yl)picolinamide |
| A102 | 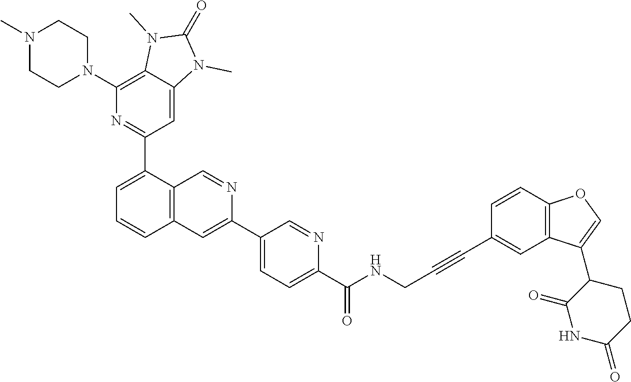 [SMILES: CN1CCN(c2nc(-c3cccc4cc(-c5ccc(C(=O)NCC#Cc6ccc7occ(C8CCC(=O)NC8=O)c7c6)nc5)ncc34)cc3c2n(C)c(=O)n3C)CC1] | 5-(8-(1,3-dimethyl-4- (4-methylpiperazin- 1-yl)-2-oxo-2,3- dihydro-1H-imidazo[4,5- c]pyridin-6-yl)iso- quinolin-3-yl)-N-(3-(3- (2,6-dioxopiperidin- 3-yl)benzofuran-5- yl)prop-2-yn-1-yl) picolinamide |
| A103 | 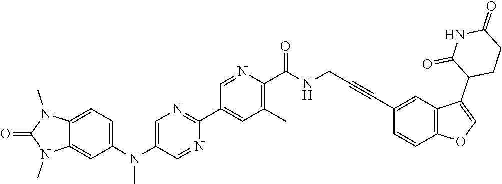 [SMILES: Cc1cc(-c2ncc(N(C)c3ccc4c(c3)n(C)c(=O)n4C)cn2)cnc1C(=O)NCC#Cc1ccc2occ(C3CCC(=O)NC3=O)c2c1] | 5-(5-((1,3-dimethyl- 2-oxo-2,3-dihydro- 1H-benzo[d]imidazol-5- yl)(methyl)amino) pyrimidin-2-yl)-N-(3- (3-(2,6-dioxopiperidin- 3-yl)benzofuran-5- yl)prop-2-yn-1-yl)-3- methylpicolinamide |
| A104 | 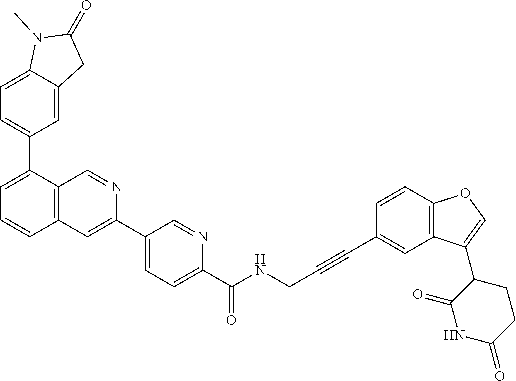 [SMILES: CN1C(=O)Cc2cc(-c3cccc4cc(-c5ccc(C(=O)NCC#Cc6ccc7occ(C8CCC(=O)NC8=O)c7c6)nc5)ncc34)ccc21] | N-(3-(3-(2,6-dioxo- piperidin-3- yl)benzofuran-5-yl) prop-2-yn-1-yl)-5-(8- (1-methyl-2-oxoindolin- 5-yl)isoquinolin- 3-yl)picolinamide |
| A105 | 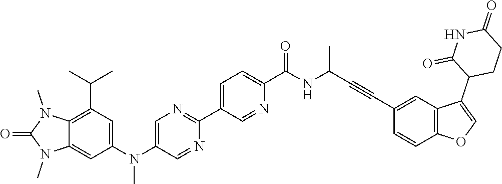 [SMILES: CC(C#Cc1ccc2occ(C3CCC(=O)NC3=O)c2c1)NC(=O)c1ccc(-c2ncc(N(C)c3cc(C(C)C)c4c(c3)n(C)c(=O)n4C)cn2)cn1] | N-(4-(3-(2,6-dioxo- piperidin-3- yl)benzofuran-5-yl) but-3-yn-2-yl)-5-(5- ((7-isopropyl-1,3- dimethyl-2-oxo-2,3- dihydro-1H-benzo [d]imidazol-5- yl)(methyl)amino) pyrimidin-2- yl)picolinamide |
| A106 | 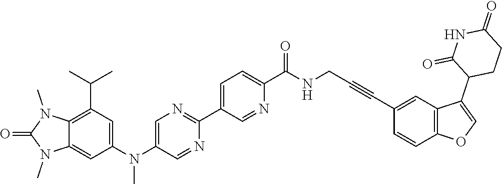 [SMILES: CC(C)c1cc(N(C)c2cnc(-c3ccc(C(=O)NCC#Cc4ccc5occ(C6CCC(=O)NC6=O)c5c4)nc3)nc2)cc2c1n(C)c(=O)n2C] | N-(3-(3-(2,6-dioxo- piperidin-3- yl)benzofuran-5-yl) prop-2-yn-1-yl)-5-(5- ((7-isopropyl-1,3- dimethyl-2-oxo-2,3- dihydro-1H-benzo [d]imidazol-5- yl)(methyl)amino) pyrimidin-2- yl)picolinamide |
| A107 | 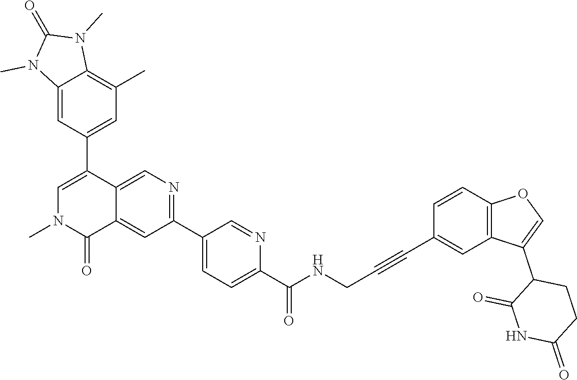 [SMILES: Cc1cc(-c2cn(C)c(=O)c3cc(-c4ccc(C(=O)NCC#Cc5ccc6occ(C7CCC(=O)NC7=O)c6c5)nc4)ncc23)cc2c1n(C)c(=O)n2C] | N-(3-(3-(2,6-dioxo- piperidin-3- yl)benzofuran-5-yl) prop-2-yn-1-yl)-5-(6- methyl-5-oxo-8- (1,3,7-trimethyl-2-oxo- 2,3-dihydro-1H- benzo[d]imidazol-5-yl)- 5,6-dihydro-2,6- naphthyridin-3- yl)picolinamide |
| A108 | 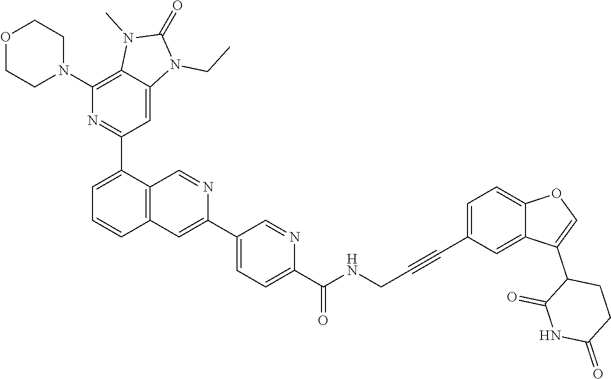 [SMILES: CCn1c(=O)n(C)c2c(N3CCOCC3)nc(-c3cccc4cc(-c5ccc(C(=O)NCC#Cc6ccc7occ(C8CCC(=O)NC8=O)c7c6)nc5)ncc34)cc21] | N-(3-(3-(2,6-dioxo- piperidin-3- yl)benzofuran-5-yl) prop-2-yn-1-yl)-5-(8- (1-ethyl-3-methyl-4- morpholino-2-oxo- 2,3-dihydro-1H- imidazo[4,5-c]pyridin- 6-yl)isoquinolin-3- yl)picolinamide |
| A110 | 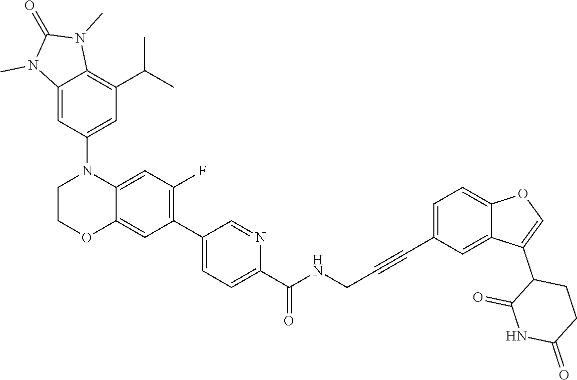 [SMILES: CC(C)c1cc(N2CCOc3cc(-c4ccc(C(=O)NCC#Cc5ccc6occ(C7CCC(=O)NC7=O)c6c5)nc4)c(F)cc32)cc2c1n(C)c(=O)n2C] | N-(3-(3-(2,6-dioxo- piperidin-3- yl)benzofuran-5-yl) prop-2-yn-1-yl)-5-(6- fluoro-4-(7-isopropyl- 1,3-dimethyl-2-oxo- 2,3-dihydro-1H- benzo[d]imidazol-5- yl)-3,4-dihydro-2H- benzo[b][1,4]oxazin- 7-yl)picolinamide |
| A111 | 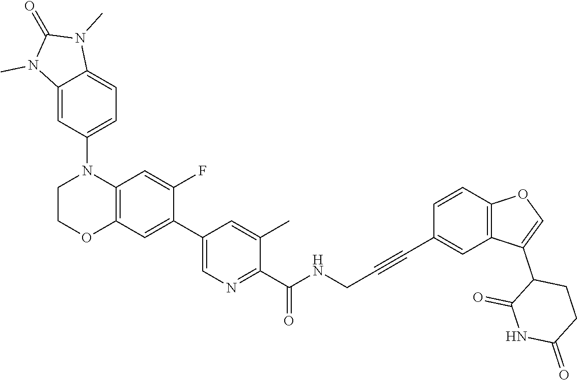 [SMILES: Cc1cc(-c2cc3c(cc2F)N(c2ccc4c(c2)n(C)c(=O)n4C)CCO3)cnc1C(=O)NCC#Cc1ccc2occ(C3CCC(=O)NC3=O)c2c1] | 5-(4-(1,3-dimethyl-2- oxo-2,3-dihydro-1H- benzo[d]imidazol-5- yl)-6-fluoro-3,4- dihydro-2H-benzo[b] [1,4]oxazin-7-yl)-N- (3-(3-(2,6-dioxo- piperidin-3- yl)benzofuran-5-yl) prop-2-yn-1-yl)-3- methylpicolinamide |
| A112 | 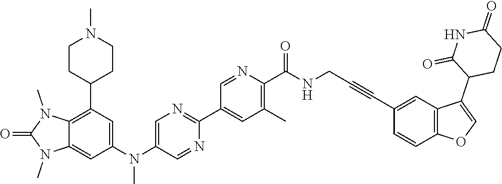 [SMILES: Cc1cc(-c2ncc(N(C)c3cc(C4CCN(C)CC4)c4c(c3)n(C)c(=O)n4C)cn2)cnc1C(=O)NCC#Cc1ccc2occ(C3CCC(=O)NC3=O)c2c1] | 5-(5-((1,3-dimethyl-7- (1-methylpiperidin- 4-yl)-2-oxo-2,3- dihydro-1H-benzo [d]imidazol-5- yl)(methyl)amino) pyrimidin-2-yl)-N-(3- (3-(2,6-dioxopiperidin- 3-yl)benzofuran-5- yl)prop-2-yn-1-yl)-3- methylpicolinamide |
| A113 | 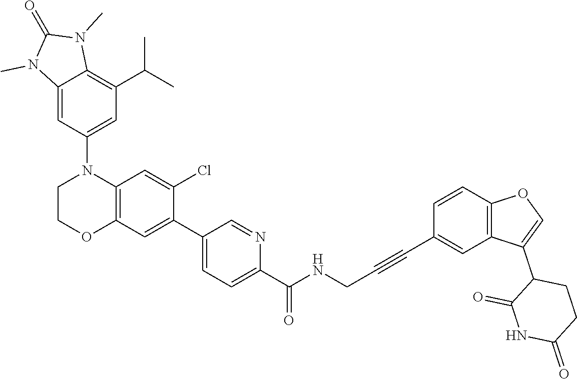 [SMILES: CC(C)c1cc(N2CCOc3cc(-c4ccc(C(=O)NCC#Cc5ccc6occ(C7CCC(=O)NC7=O)c6c5)nc4)c(Cl)cc32)cc2c1n(C)c(=O)n2C] | 5-(6-chloro-4-(7- isopropyl-1,3-dimethyl- 2-oxo-2,3-dihydro- 1H-benzo[d]imidazol- 5-yl)-3,4-dihydro-2H- benzo[b][1,4]oxazin- 7-yl)-N-(3-(3-(2,6- dioxopiperidin-3- yl)benzofuran-5- yl)prop-2-yn-1- yl)picolinamide |
| A114 | 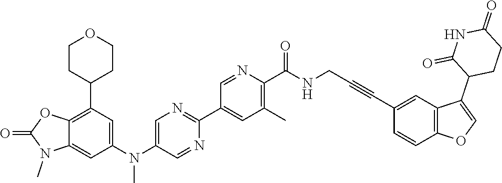 [SMILES: Cc1cc(-c2ncc(N(C)c3cc(C4CCOCC4)c4oc(=O)n(C)c4c3)cn2)cnc1C(=O)NCC#Cc1ccc2occ(C3CCC(=O)NC3=O)c2c1] | N-(3-(3-(2,6-dioxo- piperidin-3-yl) benzofuran-5-yl) prop-2-yn-1-yl)-3- methyl-5-(5-(methyl (3-methyl-2-oxo-7- (tetrahydro-2H- pyran-4-yl)-2,3- dihydrobenzo[d] oxazol-5- yl)amino)pyrimidin- 2-yl)picolinamide |
| A115 | 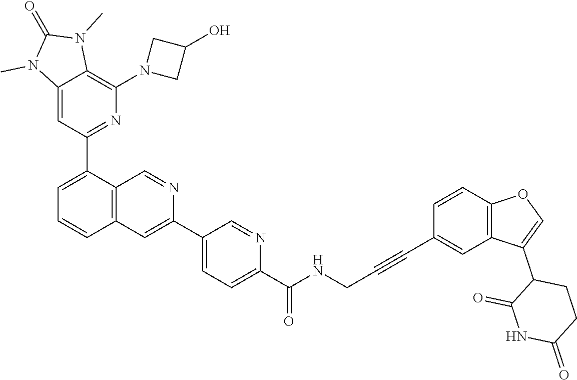 [SMILES: Cn1c(=O)n(C)c2c(N3CC(O)C3)nc(-c3cccc4cc(-c5ccc(C(=O)NCC#Cc6ccc7occ(C8CCC(=O)NC8=O)c7c6)nc5)ncc34)cc21] | N-(3-(3-(2,6-dioxo- piperidin-3- yl)benzofuran-5-yl) prop-2-yn-1-yl)-5-(8- (4-(3-hydroxyazetidin- 1-yl)-1,3-dimethyl- 2-oxo-2,3-dihydro- 1H-imidazo[4,5- c]pyridin-6-yl) isoquinolin-3- yl)picolinamide |
| A116 | 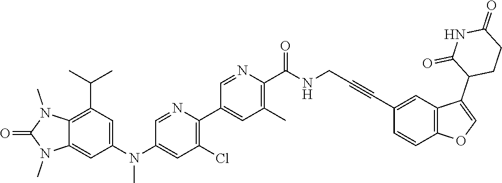 [SMILES: Cc1cc(-c2ncc(N(C)c3cc(C(C)C)c4c(c3)n(C)c(=O)n4C)cc2Cl)cnc1C(=O)NCC#Cc1ccc2occ(C3CCC(=O)NC3=O)c2c1] | 3-chloro-N-(3-(3- (2,6-dioxopiperidin-3- yl)benzofuran-5-yl) prop-2-yn-1-yl)-5-((7- isopropyl-1,3- dimethyl-2-oxo-2,3- dihydro-1H-benzo [d]imidazol-5- yl)(methyl)amino)- 5′-methyl-[2,3′- bipyridine]-6′- carboxamide |
| A117 | 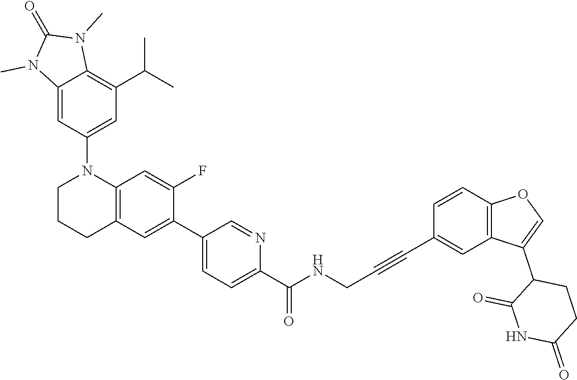 [SMILES: CC(C)c1cc(N2CCCc3cc(-c4ccc(C(=O)NCC#Cc5ccc6occ(C7CCC(=O)NC7=O)c6c5)nc4)c(F)cc32)cc2c1n(C)c(=O)n2C] | N-(3-(3-(2,6-dioxo- piperidin-3- yl)benzofuran-5-yl) prop-2-yn-1-yl)-5-(7- fluoro-1-(7-isopropyl- 1,3-dimethyl-2-oxo- 2,3-dihydro-1H-benzo [d]imidazol-5-yl)- 1,2,3,4-tetrahydro- quinolin-6- yl)picolinamide |
| A118 | 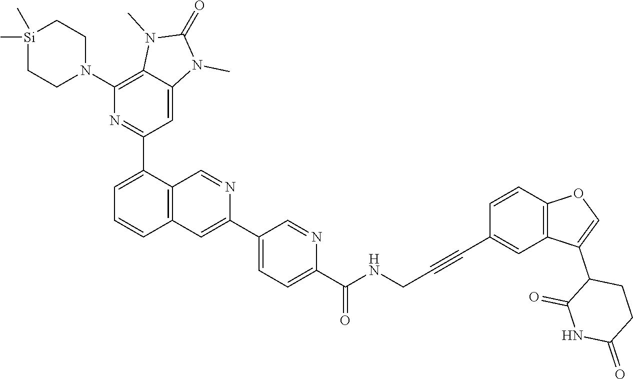 [SMILES: Cn1c(=O)n(C)c2c(N3CC[Si](C)(C)CC3)nc(-c3cccc4cc(-c5ccc(C(=O)NCC#Cc6ccc7occ(C8CCC(=O)NC8=O)c7c6)nc5)ncc34)cc21] | 5-(8-(4-(4,4-dimethyl- 1,4-azasilinan-1- yl)-1,3-dimethyl-2- oxo-2,3-dihydro-1H- imidazo[4,5-c]pyridin- 6-yl)isoquinolin-3- yl)-N-(3-(3-(2,6- dioxopiperidin-3- yl)benzofuran-5- yl)prop-2-yn-1- yl)picolinamide |
| A119 | 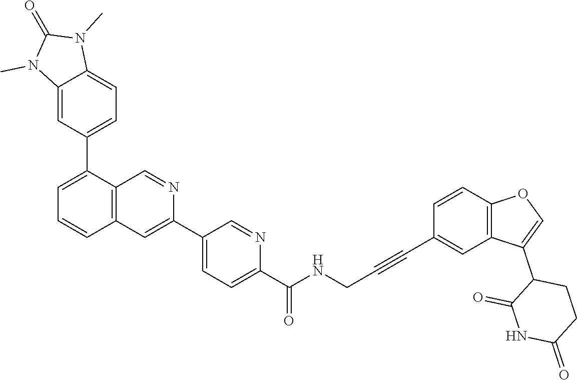 [SMILES: Cn1c(=O)n(C)c2cc(-c3cccc4cc(-c5ccc(C(=O)NCC#Cc6ccc7occ(C8CCC(=O)NC8=O)c7c6)nc5)ncc34)ccc21] | 5-(8-(1,3-dimethyl-2- oxo-2,3-dihydro-1H- benzo[d]imidazol-5- yl)isoquinolin-3-yl)- N-(3-(3-(2,6-dioxo- piperidin-3- yl)benzofuran-5- yl)prop-2-yn-1- yl)picolinamide |
| A120 | 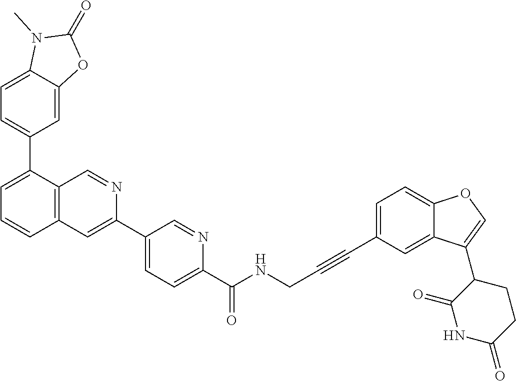 [SMILES: Cn1c(=O)oc2cc(-c3cccc4cc(-c5ccc(C(=O)NCC#Cc6ccc7occ(C8CCC(=O)NC8=O)c7c6)nc5)ncc34)ccc21] | N-(3-(3-(2,6-dioxo- piperidin-3- yl)benzofuran-5-yl) prop-2-yn-1-yl)-5-(8- (3-methyl-2-oxo-2,3- dihydrobenzo[d] oxazol-6-yl)isoquinolin- 3-yl)picolinamide |
| A121 | 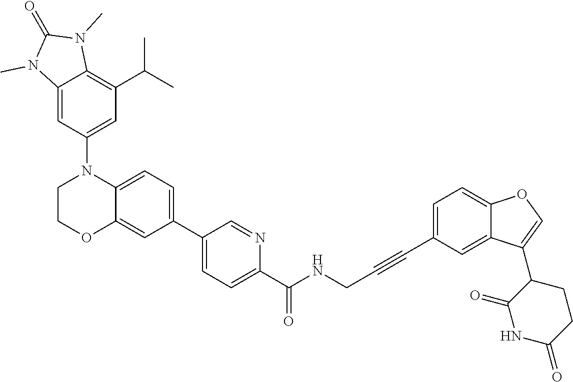 [SMILES: CC(C)c1cc(N2CCOc3cc(-c4ccc(C(=O)NCC#Cc5ccc6occ(C7CCC(=O)NC7=O)c6c5)nc4)ccc32)cc2c1n(C)c(=O)n2C] | N-(3-(3-(2,6-dioxo- piperidin-3- yl)benzofuran-5-yl) prop-2-yn-1-yl)-5-(4- (7-isopropyl-1,3- dimethyl-2-oxo-2,3- dihydro-1H-benzo [d]imidazol-5-yl)-3,4- dihydro-2H-benzo [b][1,4]oxazin-7- yl)picolinamide |
| A122 | 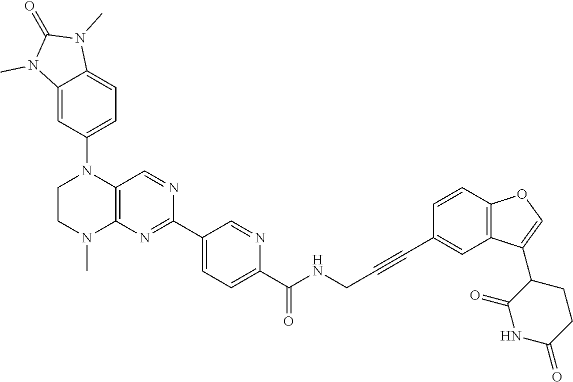 [SMILES: CN1CCN(c2ccc3c(c2)n(C)c(=O)n3C)c2cnc(-c3ccc(C(=O)NCC#Cc4ccc5occ(C6CCC(=O)NC6=O)c5c4)nc3)nc21] | 5-(5-(1,3-dimethyl-2- oxo-2,3-dihydro-1H- benzo[d]imidazol-5- yl)-8-methyl-5,6,7,8- tetrahydropteridin-2- yl)-N-(3-(3-(2,6- dioxopiperidin-3- yl)benzofuran-5- yl)prop-2-yn-1- yl)picolinamide |
| A123 | 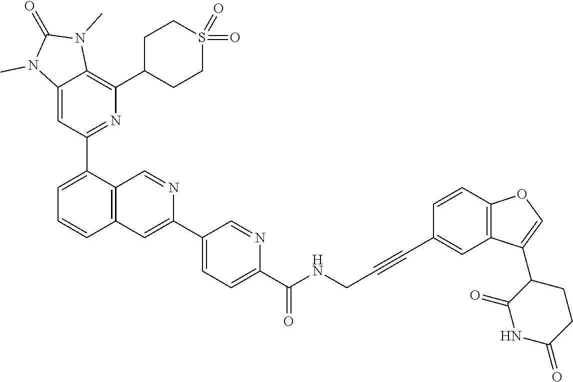 [SMILES: Cn1c(=O)n(C)c2c(C3CCS(=O)(=O)CC3)nc(-c3cccc4cc(-c5ccc(C(=O)NCC#Cc6ccc7occ(C8CCC(=O)NC8=O)c7c6)nc5)ncc34)cc21] | 5-(8-(4-(1,1-dioxido- tetrahydro-2H- thiopyran-4-yl)-1,3- dimethyl-2-oxo-2,3- dihydro-1H-imidazo [4,5-c]pyridin-6- yl)isoquinolin-3- yl)-N-(3-(3-(2,6- dioxopiperidin-3- yl)benzofuran-5- yl)prop-2-yn-1- yl)picolinamide |
| A124 | 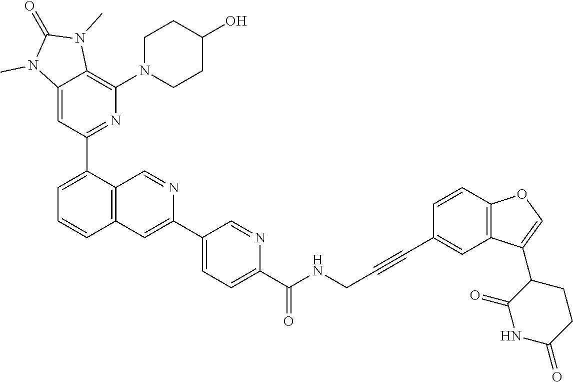 [SMILES: Cn1c(=O)n(C)c2c(N3CCC(O)CC3)nc(-c3cccc4cc(-c5ccc(C(=O)NCC#Cc6ccc7occ(C8CCC(=O)NC8=O)c7c6)nc5)ncc34)cc21] | N-(3-(3-(2,6-dioxo- piperidin-3- yl)benzofuran-5-yl) prop-2-yn-1-yl)-5-(8- (4-(4-hydroxypiperidin- 1-yl)-1,3- dimethyl-2-oxo-2,3- dihydro-1H- imidazo[4,5-c] pyridin-6-yl)isoquinolin- 3-yl)picolinamide |
| A125 | 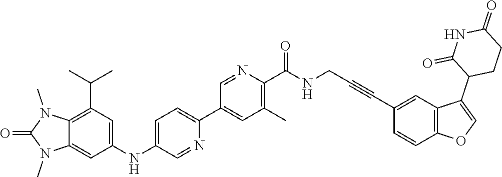 [SMILES: Cc1cc(-c2ccc(Nc3cc(C(C)C)c4c(c3)n(C)c(=O)n4C)cn2)cnc1C(=O)NCC#Cc1ccc2occ(C3CCC(=O)NC3=O)c2c1] | N-(3-(3-(2,6-dioxo- piperidin-3- yl)benzofuran-5-yl) prop-2-yn-1-yl)-5-((7- isopropyl-1,3- dimethyl-2-oxo-2,3- dihydro-1H-benzo [d]imidazol-5- yl)amino)-5′-methyl- [2,3′-bipyridine]-6′- carboxamide |
| A126 | 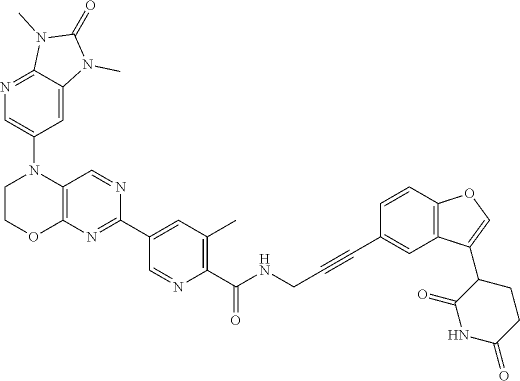 [SMILES: Cc1cc(-c2ncc3c(n2)OCCN3c2cnc3c(c2)n(C)c(=O)n3C)cnc1C(=O)NCC#Cc1ccc2occ(C3CCC(=O)NC3=O)c2c1] | 5-(5-(1,3-dimethyl-2- oxo-2,3-dihydro-1H- imidazo[4,5-b]pyridin- 6-yl)-6,7-dihydro- 5H-pyrimido[4,5-b] [1,4]oxazin-2-yl)-N- (3-(3-(2,6-dioxo- piperidin-3- yl)benzofuran-5-yl) prop-2-yn-1-yl)-3- methylpicolinamide |
| A127 | 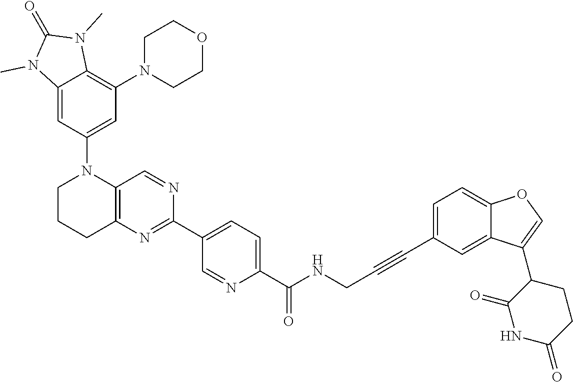 [SMILES: Cn1c(=O)n(C)c2c(N3CCOCC3)cc(N3CCCc4nc(-c5ccc(C(=O)NCC#Cc6ccc7occ(C8CCC(=O)NC8=O)c7c6)nc5)ncc43)cc21] | 5-(5-(1,3-dimethyl-7- morpholino-2-oxo- 2,3-dihydro-1H-benzo [d]imidazol-5-yl)- 5,6,7,8-tetrahydro- pyrido[3,2-d]pyrimidin- 2-yl)-N-(3-(3-(2,6- dioxopiperidin-3- yl)benzofuran-5- yl)prop-2-yn-1- yl)picolinamide |
| A128 | 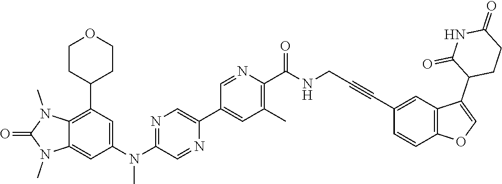 [SMILES: Cc1cc(-c2cnc(N(C)c3cc(C4CCOCC4)c4c(c3)n(C)c(=O)n4C)cn2)cnc1C(=O)NCC#Cc1ccc2occ(C3CCC(=O)NC3=O)c2c1] | 5-(5-((1,3-dimethyl- 2-oxo-7-(tetrahydro- 2H-pyran-4-yl)- 2,3-dihydro-1H- benzo[d]imidazol-5- yl)(methyl)amino) pyrazin-2-yl)-N-(3-(3- (2,6-dioxopiperidin- 3-yl)benzofuran-5- yl)prop-2-yn-1-yl)- 3-methylpicolinamide |
| A129 | 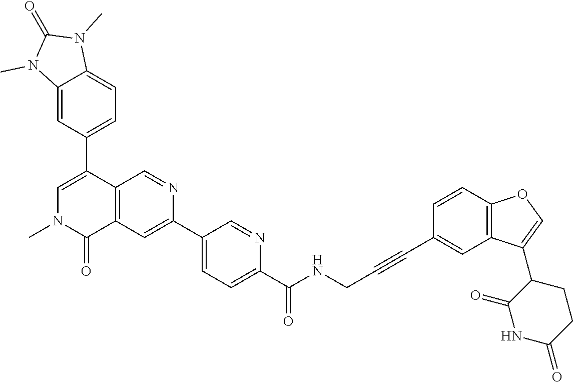 [SMILES: Cn1cc(-c2ccc3c(c2)n(C)c(=O)n3C)c2cnc(-c3ccc(C(=O)NCC#Cc4ccc5occ(C6CCC(=O)NC6=O)c5c4)nc3)cc2c1=O] | 5-(8-(1,3-dimethyl- 2-oxo-2,3-dihydro-1H- benzo[d]imidazol- 5-yl)-6-methyl-5-oxo- 5,6-dihydro-2,6- naphthyridin-3-yl)- N-(3-(3-(2,6-dioxo- piperidin-3-yl)benzo- furan-5-yl)prop-2-yn- 1-yl)picolinamide |
| A130 | 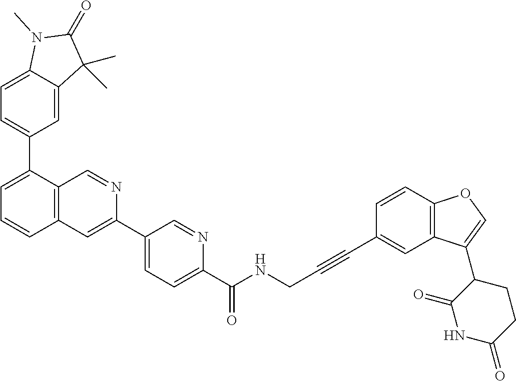 [SMILES: CN1C(=O)C(C)(C)c2cc(-c3cccc4cc(-c5ccc(C(=O)NCC#Cc6ccc7occ(C8CCC(=O)NC8=O)c7c6)nc5)ncc34)ccc21] | N-(3-(3-(2,6-dioxo- piperidin-3-yl)benzo- furan-5-yl)prop-2- yn-1-yl)-5-(8- (1,3,3-trimethyl- 2-oxoindolin-5- yl)isoquinolin-3- yl)picolinamide |
| A132 | 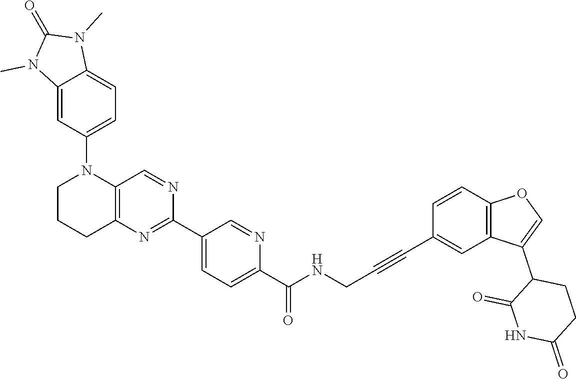 [SMILES: Cn1c(=O)n(C)c2cc(N3CCCc4nc(-c5ccc(C(=O)NCC#Cc6ccc7occ(C8CCC(=O)NC8=O)c7c6)nc5)ncc43)ccc21] | 5-(5-(1,3-dimethyl-2- oxo-2,3-dihydro-1H- benzo[d]imidazol-5- yl)-5,6,7,8- tetrahydropyrido[3,2- d]pyrimidin-2-yl)- N-(3-(3-(2,6-dioxo- piperidin-3- yl)benzofuran-5-yl) prop-2-yn-1- yl)picolinamide |
| A133 | 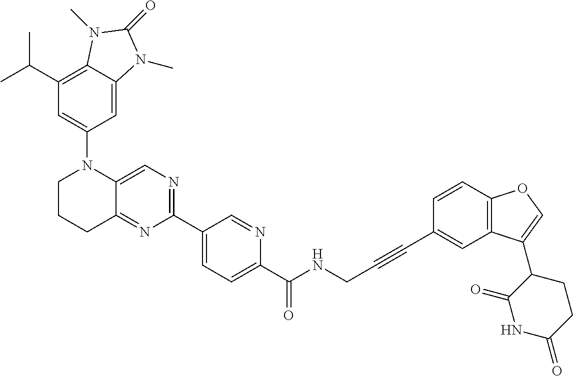 [SMILES: CC(C)c1cc(N2CCCc3nc(-c4ccc(C(=O)NCC#Cc5ccc6occ(C7CCC(=O)NC7=O)c6c5)nc4)ncc32)cc2c1n(C)c(=O)n2C] | N-(3-(3-(2,6-dioxo- piperidin-3- yl)benzofuran-5-yl) prop-2-yn-1-yl)-5-(5- (7-isopropyl-1,3- dimethyl-2-oxo-2,3- dihydro-1H-benzo [d]imidazol-5-yl)- 5,6,7,8-tetrahydro- pyrido[3,2-d]pyrimidin- 2-yl)picolinamide |
| A134 | 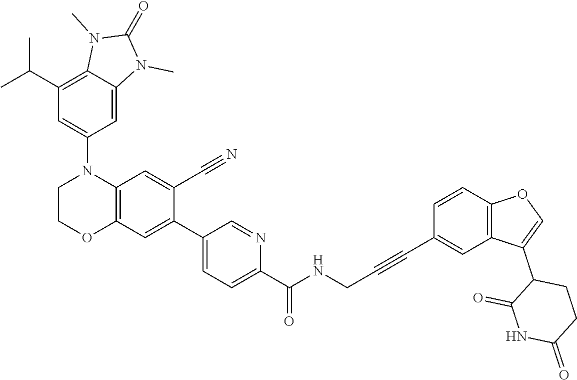 [SMILES: CC(C)c1cc(N2CCOc3cc(-c4ccc(C(=O)NCC#Cc5ccc6occ(C7CCC(=O)NC7=O)c6c5)nc4)c(C#N)cc32)cc2c1n(C)c(=O)n2C] | 5-(6-cyano-4-(7-iso- propyl-1,3-dimethyl- 2-oxo-2,3-dihydro- 1H-benzo[d]imidazol- 5-yl)-3,4-dihydro-2H- benzo[b][1,4]oxazin- 7-yl)-N-(3-(3-(2,6- dioxopiperidin-3- yl)benzofuran-5- yl)prop-2-yn-1- yl)picolinamide |
| A135 | 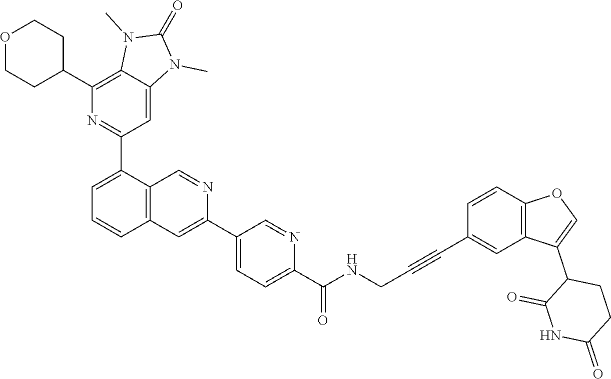 [SMILES: Cn1c(=O)n(C)c2c(C3CCOCC3)nc(-c3cccc4cc(-c5ccc(C(=O)NCC#Cc6ccc7occ(C8CCC(=O)NC8=O)c7c6)nc5)ncc34)cc21] | 5-(8-(1,3-dimethyl- 2-oxo-4-(tetrahydro- 2H-pyran-4-yl)-2,3- dihydro-1H- imidazo[4,5-c]pyridin- 6-yl)isoquinolin-3- yl)-N-(3-(3-(2,6- dioxopiperidin-3- yl)benzofuran-5- yl)prop-2-yn-1- yl)picolinamide |
| A136 | 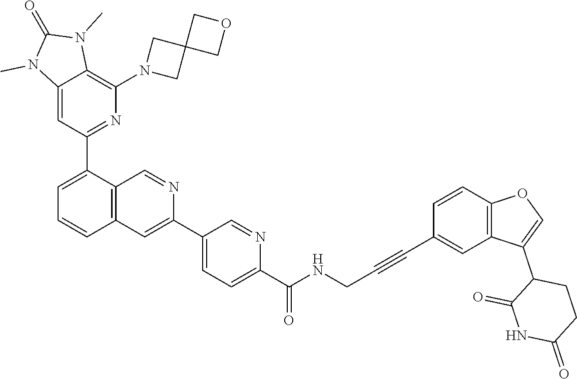 [SMILES: Cn1c(=O)n(C)c2c(N3CC4(COC4)C3)nc(-c3cccc4cc(-c5ccc(C(=O)NCC#Cc6ccc7occ(C8CCC(=O)NC8=O)c7c6)nc5)ncc34)cc21] | 5-(8-(1,3-dimethyl- 2-oxo-4-(2-oxa-6- azaspiro[3.3]heptan- 6-yl)-2,3-dihydro- 1H-imidazo[4,5- c]pyridin-6- yl)isoquinolin-3- yl)-N-(3-(3-(2,6- dioxopiperidin-3- yl)benzofuran-5- yl)prop-2-yn-1- yl)picolinamide |
| A137 | 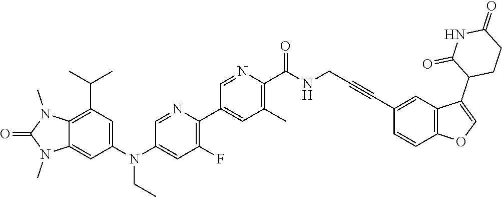 [SMILES: CCN(c1cnc(-c2cnc(C(=O)NCC#Cc3ccc4occ(C5CCC(=O)NC5=O)c4c3)c(C)c2)c(F)c1)c1cc(C(C)C)c2c(c1)n(C)c(=O)n2C] | N-(3-(3-(2,6-dioxo- piperidin-3- yl)benzofuran-5-yl) prop-2-yn-1-yl)-5- (ethyl(7-isopropyl- 1,3-dimethyl-2-oxo- 2,3-dihydro-1H- benzo[d]imidazol-5- yl)amino)-3-fluoro- 5′-methyl-[2,3′- bipyridine]-6′- carboxamide |
| A138 | 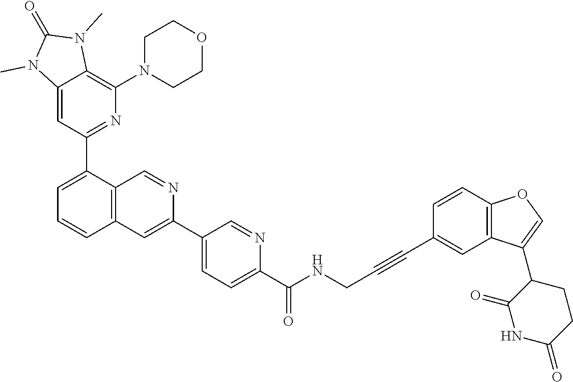 [SMILES: Cn1c(=O)n(C)c2c(N3CCOCC3)nc(-c3cccc4cc(-c5ccc(C(=O)NCC#Cc6ccc7occ(C8CCC(=O)NC8=O)c7c6)nc5)ncc34)cc21] | 5-(8-(1,3-dimethyl- 4-morpholino-2-oxo- 2,3-dihydro-1H- imidazo[4,5-c]pyridin- 6-yl)isoquinolin-3-yl)- N-(3-(3-(2,6- dioxopiperidin-3-yl) benzofuran-5- yl)prop-2-yn-1- yl)picolinamide |
| A139 | 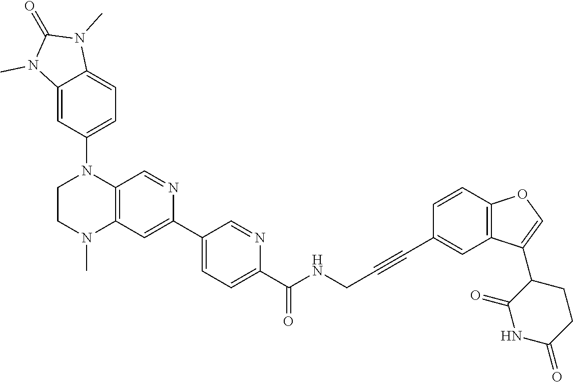 [SMILES: CN1CCN(c2ccc3c(c2)n(C)c(=O)n3C)c2cnc(-c3ccc(C(=O)NCC#Cc4ccc5occ(C6CCC(=O)NC6=O)c5c4)nc3)cc21] | 5-(4-(1,3-dimethyl-2- oxo-2,3-dihydro-1H- benzo[d]imidazol-5- yl)-1-methyl-1,2,3,4- tetrahydropyrido[3,4- b]pyrazin-7-yl)-N- (3-(3-(2,6-dioxo- piperidin-3- yl)benzofuran-5- yl)prop-2-yn-1- yl)picolinamide |
| A140 | 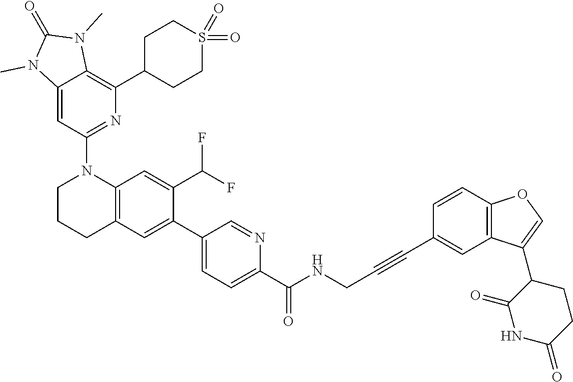 [SMILES: Cn1c(=O)n(C)c2c(C3CCS(=O)(=O)CC3)nc(N3CCCc4cc(-c5ccc(C(=O)NCC#Cc6ccc7occ(C8CCC(=O)NC8=O)c7c6)nc5)c(C(F)F)cc43)cc21] | 5-(7-(difluoromethyl)- 1-(4-(1,1-dioxido- tetrahydro-2H-thio- pyran-4-yl)-1,3- dimethyl-2-oxo- 2,3-dihydro-1H- imidazo[4,5-c] pyridin-6-yl)-1,2,3,4- tetrahydroquinolin- 6-yl)-N-(3-(3-(2,6- dioxopiperidin-3- yl)benzofuran-5- yl)prop-2-yn-1- yl)picolinamide |
| A141 | 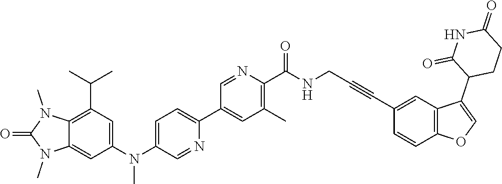 [SMILES: Cc1cc(-c2ccc(N(C)c3cc(C(C)C)c4c(c3)n(C)c(=O)n4C)cn2)cnc1C(=O)NCC#Cc1ccc2occ(C3CCC(=O)NC3=O)c2c1] | N-(3-(3-(2,6-dioxo- piperidin-3-yl) benzofuran-5-yl)prop- 2-yn-1-yl)-5-((7- isopropyl-1,3- dimethyl-2-oxo-2,3- dihydro-1H-benzo [d]imidazol-5- yl)(methyl)amino)- 5′-methyl-[2,3′- bipyridine]-6′- carboxamide |
| A142 | 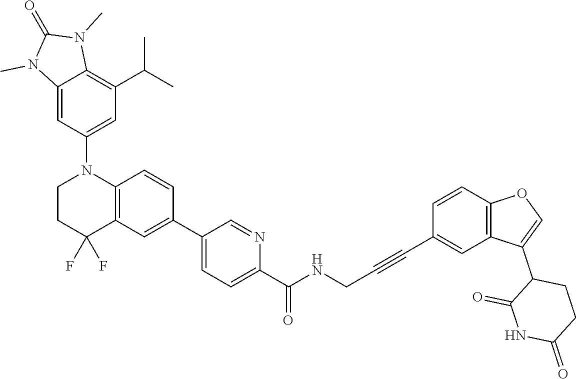 [SMILES: CC(C)c1cc(N2CCC(F)(F)c3cc(-c4ccc(C(=O)NCC#Cc5ccc6occ(C7CCC(=O)NC7=O)c6c5)nc4)ccc32)cc2c1n(C)c(=O)n2C] | 5-(4,4-difluoro-1- (7-isopropyl-1,3- dimethyl-2-oxo- 2,3-dihydro-1H- benzo[d]imidazol- 5-yl)-1,2,3,4- tetrahydroquinolin- 6-yl)-N-(3-(3-(2,6- dioxopiperidin-3- yl)benzofuran-5- yl)prop-2-yn-1- yl)picolinamide |
| A143 | 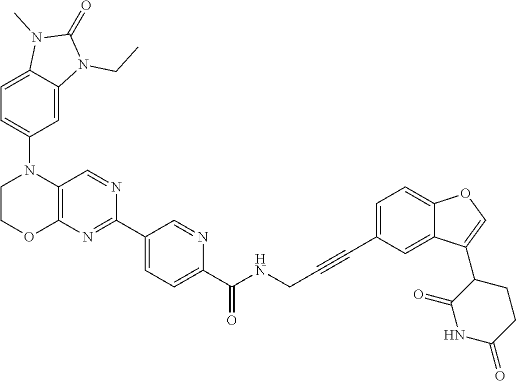 [SMILES: CCn1c(=O)n(C)c2ccc(N3CCOc4nc(-c5ccc(C(=O)NCC#Cc6ccc7occ(C8CCC(=O)NC8=O)c7c6)nc5)ncc43)cc21] | N-(3-(3-(2,6-dioxo- piperidin-3- yl)benzofuran-5-yl) prop-2-yn-1-yl)-5-(5- (3-ethyl-1-methyl-2- oxo-2,3-dihydro-1H- benzo[d]imidazol-5- yl)-6,7-dihydro-5H- pyrimido[4,5-b] [1,4]oxazin-2- yl)picolinamide |
| A144 | 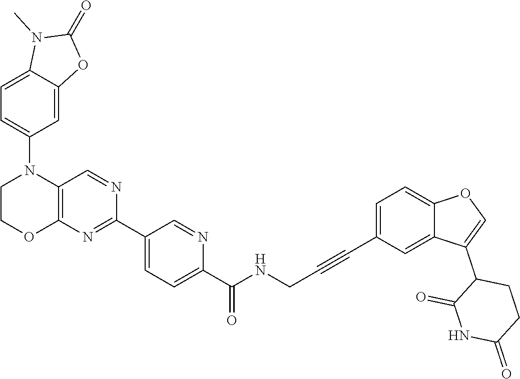 [SMILES: Cn1c(=O)oc2cc(N3CCOc4nc(-c5ccc(C(=O)NCC#Cc6ccc7occ(C8CCC(=O)NC8=O)c7c6)nc5)ncc43)ccc21] | N-(3-(3-(2,6-dioxo- piperidin-3- yl)benzofuran-5-yl) prop-2-yn-1-yl)-5-(5- (3-methyl-2-oxo-2,3- dihydrobenzo[d] oxazol-6-yl)-6,7- dihydro-5H-pyrimido [4,5-b][1,4]oxazin-2- yl)picolinamide |
| A145 | 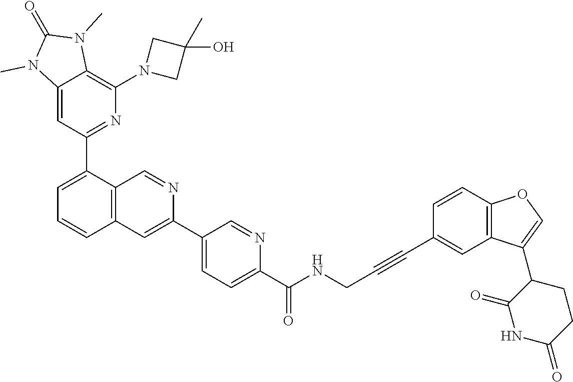 [SMILES: Cn1c(=O)n(C)c2c(N3CC(C)(O)C3)nc(-c3cccc4cc(-c5ccc(C(=O)NCC#Cc6ccc7occ(C8CCC(=O)NC8=O)c7c6)nc5)ncc34)cc21] | N-(3-(3-(2,6-dioxo- piperidin-3- yl)benzofuran-5-yl) prop-2-yn-1-yl)-5-(8- (4-(3-hydroxy-3- methylazetidin-1-yl)- 1,3-dimethyl-2- oxo-2,3-dihydro-1H- imidazo[4,5-c]pyridin- 6-yl)isoquinolin-3- yl)picolinamide |
| A146 | 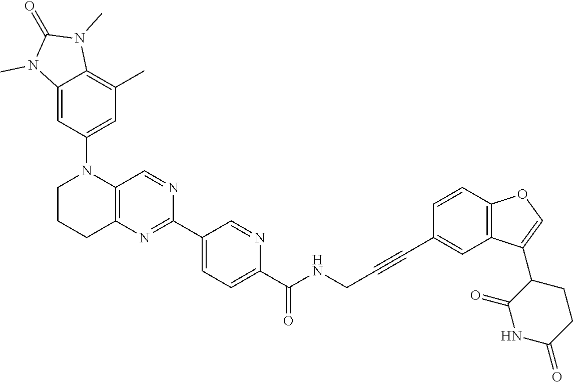 [SMILES: Cc1cc(N2CCCc3nc(-c4ccc(C(=O)NCC#Cc5ccc6occ(C7CCC(=O)NC7=O)c6c5)nc4)ncc32)cc2c1n(C)c(=O)n2C] | N-(3-(3-(2,6-dioxo- piperidin-3- yl)benzofuran-5-yl) prop-2-yn-1-yl)-5-(5- (1,3,7-trimethyl-2- oxo-2,3-dihydro-1H- benzo[d]imidazol- 5-yl)-5,6,7,8- tetrahydropyrido [3,2-d]pyrimidin-2- yl)picolinamide |
| A147 | 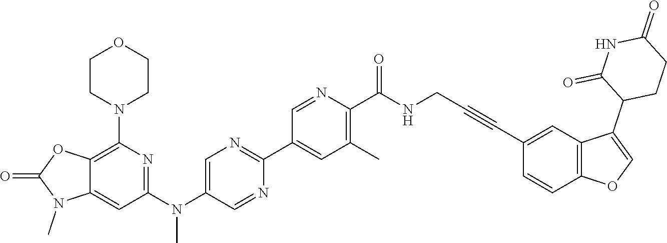 [SMILES: Cc1cc(-c2ncc(N(C)c3cc4c(oc(=O)n4C)c(N4CCOCC4)n3)cn2)cnc1C(=O)NCC#Cc1ccc2occ(C3CCC(=O)NC3=O)c2c1] | 5-(5-((1,3-dimethyl- 4-morpholino-2-oxo- 2,3-dihydro-1H- imidazo[4,5-c]pyridin- 6-yl)(methyl)amino) pyrimidin-2-yl)-N-(3- (3-(2,6-dioxopiperidin- 3-yl)benzofuran-5- yl)prop-2-yn-1-yl)-3- methylpicolinamide |
| A148 | 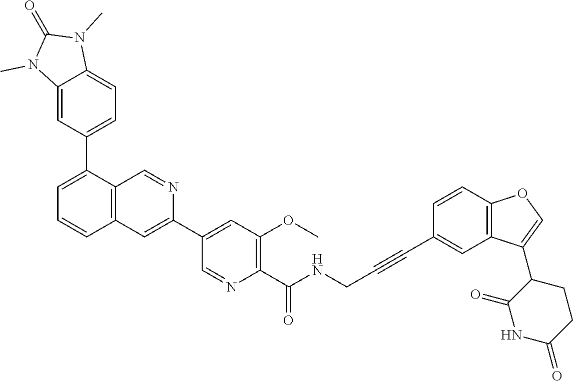 [SMILES: COc1cc(-c2cc3cccc(-c4ccc5c(c4)n(C)c(=O)n5C)c3cn2)cnc1C(=O)NCC#Cc1ccc2occ(C3CCC(=O)NC3=O)c2c1] | 5-(8-(1,3-dimethyl-2- oxo-2,3-dihydro-1H- benzo[d]imidazol-5- yl)isoquinolin-3-yl)- N-(3-(3-(2,6-dioxo- piperidin-3- yl)benzofuran-5-yl) prop-2-yn-1-yl)-3- methoxypicolinamide |
| A149 | 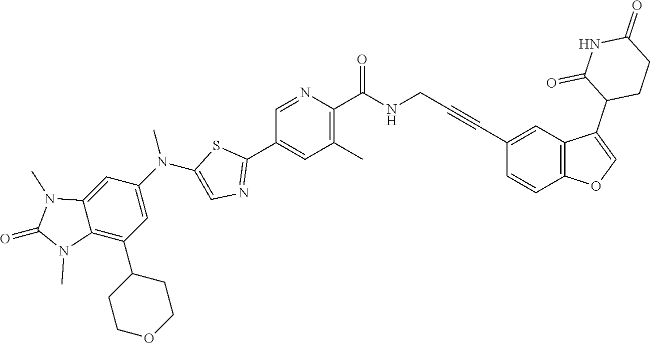 [SMILES: Cc1cc(-c2ncc(N(C)c3cc(C4CCOCC4)c4c(c3)n(C)c(=O)n4C)s2)cnc1C(=O)NCC#Cc1ccc2occ(C3CCC(=O)NC3=O)c2c1] | 5-(5-((1,3-dimethyl- 2-oxo-7-(tetrahydro- 2H-pyran-4-yl)-2,3- dihydro-1H- benzo[d]imidazol-5- yl)(methyl)amino) thiazol-2-yl)-N-(3-(3- (2,6-dioxopiperidin- 3-yl)benzofuran-5- yl)prop-2-yn-1-yl)- 3-methylpicolinamide |
| A150 | 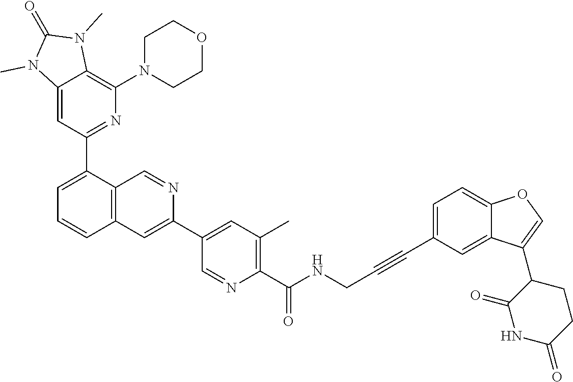 [SMILES: Cc1cc(-c2cc3cccc(-c4cc5c(c(N6CCOCC6)n4)n(C)c(=O)n5C)c3cn2)cnc1C(=O)NCC#Cc1ccc2occ(C3CCC(=O)NC3=O)c2c1] | 5-(8-(1,3-dimethyl- 4-morpholino-2-oxo- 2,3-dihydro-1H- imidazo[4,5-c]pyridin- 6-yl)isoquinolin- 3-yl)-N-(3-(3-(2,6- dioxopiperidin-3- yl)benzofuran-5- yl)prop-2-yn-1-yl)- 3-methylpicolinamide |
| A151 | 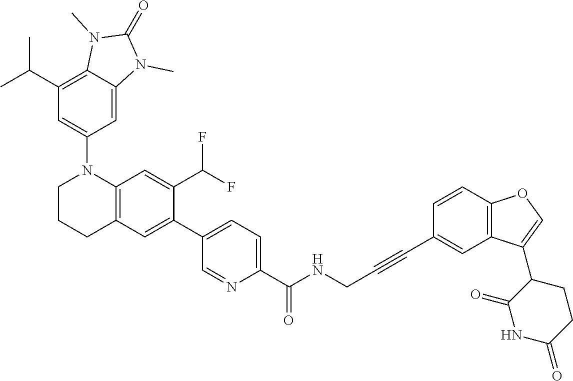 [SMILES: CC(C)c1cc(N2CCCc3cc(-c4ccc(C(=O)NCC#Cc5ccc6occ(C7CCC(=O)NC7=O)c6c5)nc4)c(C(F)F)cc32)cc2c1n(C)c(=O)n2C] | 5-(7-(difluoromethyl)- 1-(7-isopropyl-1,3- dimethyl-2-oxo- 2,3-dihydro-1H- benzo[d]imidazol- 5-yl)-1,2,3,4- tetrahydroquinolin- 6-yl)-N-(3-(3-(2,6- dioxopiperidin-3- yl)benzofuran-5- yl)prop-2-yn-1- yl)picolinamide |
| A152 | 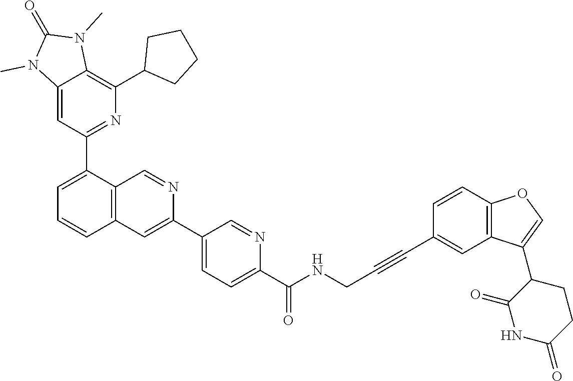 [SMILES: Cn1c(=O)n(C)c2c(C3CCCC3)nc(-c3cccc4cc(-c5ccc(C(=O)NCC#Cc6ccc7occ(C8CCC(=O)NC8=O)c7c6)nc5)ncc34)cc21] | 5-(8-(1,3-dimethyl-2- oxo-4-(1H-pyrazol- 1-yl)-2,3-dihydro- 1H-imidazo[4,5- c]pyridin-6-yl)iso- quinolin-3-yl)-N-(3-(3- (2,6-dioxopiperidin- 3-yl)benzofuran-5- yl)prop-2-yn-1- yl)picolinamide |
| A153 | 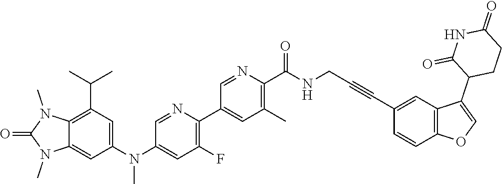 [SMILES: Cc1cc(-c2ncc(N(C)c3cc(C(C)C)c4c(c3)n(C)c(=O)n4C)cc2F)cnc1C(=O)NCC#Cc1ccc2occ(C3CCC(=O)NC3=O)c2c1] | N-(3-(3-(2,6-dioxo- piperidin-3- yl)benzofuran-5-yl) prop-2-yn-1-yl)-3- fluoro-5-((7-iso- propyl-1,3-dimethyl-2- oxo-2,3-dihydro- 1H-benzo[d]imidazol-5- yl)(methyl)amino)- 5′-methyl-[2,3′- bipyridine]-6′- carboxamide |
| A154 | 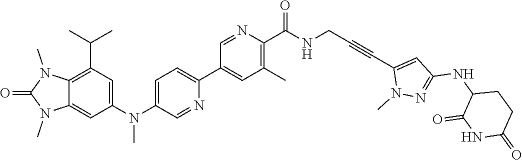 [SMILES: Cc1cc(-c2ccc(N(C)c3cc(C(C)C)c4c(c3)n(C)c(=O)n4C)cn2)cnc1C(=O)NCC#Cc1cc(NC2CCC(=O)NC2=O)nn1C] | N-(3-(3-((2,6-dioxo- piperidin-3-yl)amino)- 1-methyl-1H-pyrazol- 5-yl)prop-2-yn-1- yl)-5-((7-isopropyl- 1,3-dimethyl-2-oxo- 2,3-dihydro-1H-benzo [d]imidazol-5-yl) (methyl)amino)-5′-methyl- [2,3′-bipyridine]-6′- carboxamide |
| A155 | 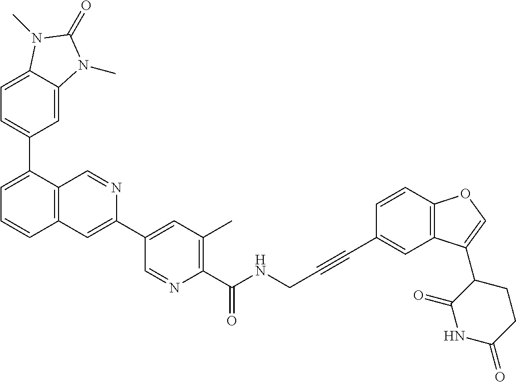 [SMILES: Cc1cc(-c2cc3cccc(-c4ccc5c(c4)n(C)c(=O)n5C)c3cn2)cnc1C(=O)NCC#Cc1ccc2occ(C3CCC(=O)NC3=O)c2c1] | 5-(8-(1,3-dimethyl-2- oxo-2,3-dihydro-1H- benzo[d]imidazol-5- yl)isoquinolin-3-yl)- N-(3-(3-(2,6-dioxo- piperidin-3-yl)benzofuran- 5-yl)prop-2-yn-1-yl)-3- methylpicolinamide |
| A156 | 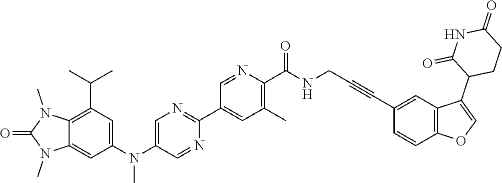 [SMILES: Cc1cc(-c2ncc(N(C)c3cc(C(C)C)c4c(c3)n(C)c(=O)n4C)cn2)cnc1C(=O)NCC#Cc1ccc2occ(C3CCC(=O)NC3=O)c2c1] | N-(3-(3-(2,6-dioxo- piperidin-3- yl)benzofuran-5-yl) prop-2-yn-1-yl)-5-(5- ((7-isopropyl-1,3- dimethyl-2-oxo-2,3- dihydro-1H-benzo [d]imidazol-5- yl)(methyl)amino) pyrimidin-2-yl)-3- methylpicolinamide |
| A157 | 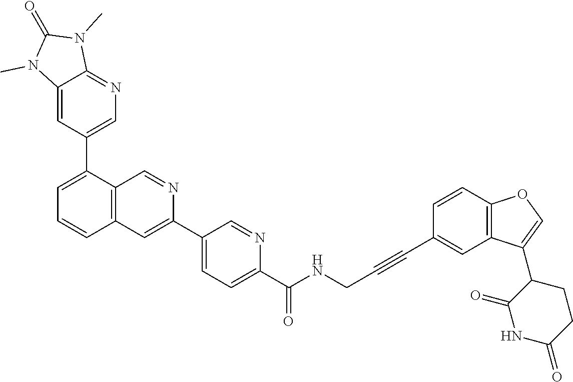 [SMILES: Cn1c(=O)n(C)c2ncc(-c3cccc4cc(-c5ccc(C(=O)NCC#Cc6ccc7occ(C8CCC(=O)NC8=O)c7c6)nc5)ncc34)cc21] | 5-(8-(1,3-dimethyl-2- oxo-2,3-dihydro-1H- imidazo[4,5-b]pyridin- 6-yl)isoquinolin-3- yl)-N-(3-(3-(2,6- dioxopiperidin-3- yl)benzofuran-5-yl) prop-2-yn-1- yl)picolinamide |
| A158 | 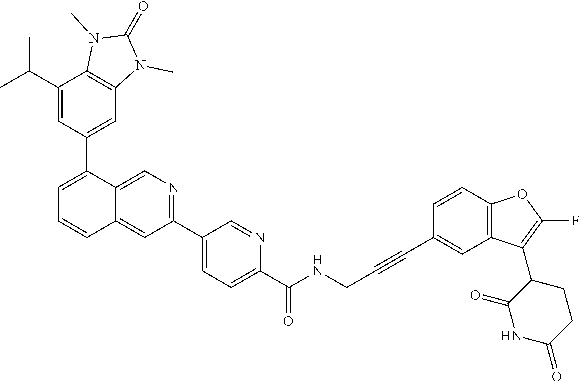 [SMILES: CC(C)c1cc(-c2cccc3cc(-c4ccc(C(=O)NCC#Cc5ccc6oc(F)c(C7CCC(=O)NC7=O)c6c5)nc4)ncc23)cc2c1n(C)c(=O)n2C] | N-(3-(3-(2,6-dioxo- piperidin-3-yl)-2- fluorobenzofuran-5- yl)prop-2-yn-1-yl)-5- (8-(7-isopropyl-1,3- dimethyl-2-oxo-2,3- dihydro-1H-benzo [d]imidazol-5- yl)isoquinolin-3- yl)picolinamide |
| A159 | 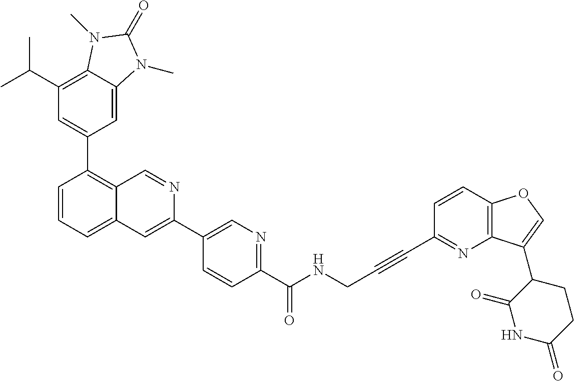 [SMILES: CC(C)c1cc(-c2cccc3cc(-c4ccc(C(=O)NCC#Cc5ccc6occ(C7CCC(=O)NC7=O)c6n5)nc4)ncc23)cc2c1n(C)c(=O)n2C] | N-(3-(3-(2,6-dioxo- piperidin-3-yl)furo[3,2- b]pyridin-5-yl)prop-2- yn-1-yl)-5-(8-(7- isopropyl-1,3- dimethyl-2-oxo-2,3- dihydro-1H-benzo [d]imidazol-5- yl)isoquinolin-3- yl)picolinamide |
| A160 | 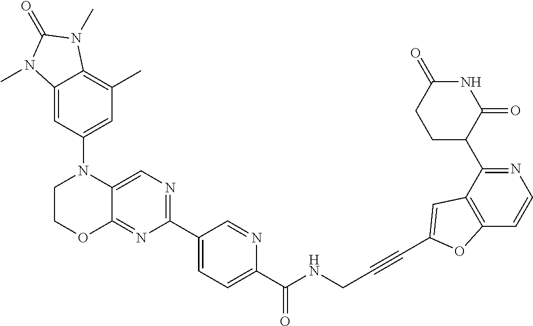 [SMILES: Cc1cc(N2CCOc3nc(-c4ccc(C(=O)NCC#Cc5cc6c(C7CCC(=O)NC7=O)nccc6o5)nc4)ncc32)cc2c1n(C)c(=O)n2C] | N-(3-(4-(2,6-dioxo- piperidin-3-yl)furo[3,2- c]pyridin-2-yl)prop- 2-yn-1-yl)-5-(5- (1,3,7-trimethyl-2- oxo-2,3-dihydro-1H- benzo[d]imidazol-5- yl)-6,7-dihydro-5H- pyrimido[4,5-b] [1,4]oxazin-2- yl)picolinamide |
| A161 | 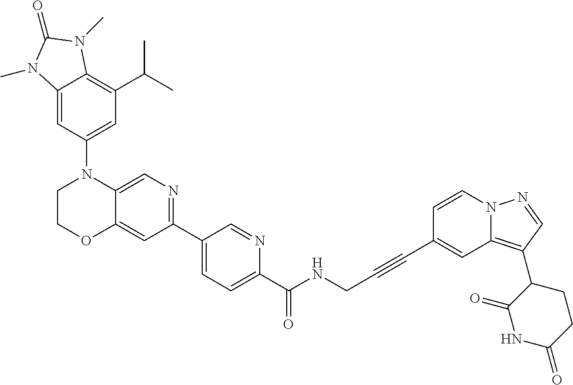 [SMILES: CC(C)c1cc(N2CCOc3cc(-c4ccc(C(=O)NCC#Cc5ccn6ncc(C7CCC(=O)NC7=O)c6c5)nc4)ncc32)cc2c1n(C)c(=O)n2C] | N-(3-(3-(2,6-dioxo- piperidin-3- yl)pyrazolo[1,5-a] pyridin-5-yl)prop-2-yn- 1-yl)-5-(4-(7-isopropyl- 1,3-dimethyl-2- oxo-2,3-dihydro-1H- benzo[d]imidazol-5- yl)-3,4-dihydro-2H- pyrido[4,3-b][1,4] oxazin-7-yl)picolinamide |
| A162 | 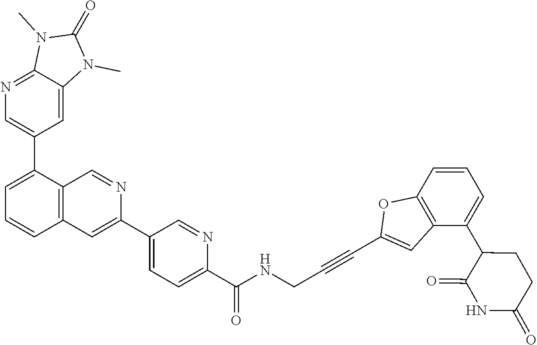 [SMILES: Cn1c(=O)n(C)c2ncc(-c3cccc4cc(-c5ccc(C(=O)NCC#Cc6cc7c(C8CCC(=O)NC8=O)cccc7o6)nc5)ncc34)cc21] | 5-(8-(1,3-dimethyl-2- oxo-2,3-dihydro-1H- imidazo[4,5-b]pyridin- 6-yl)isoquinolin-3- yl)-N-(3-(4-(2,6- dioxopiperidin-3- yl)benzofuran-2- yl)prop-2-yn-1- yl)picolinamide |
| A163 | 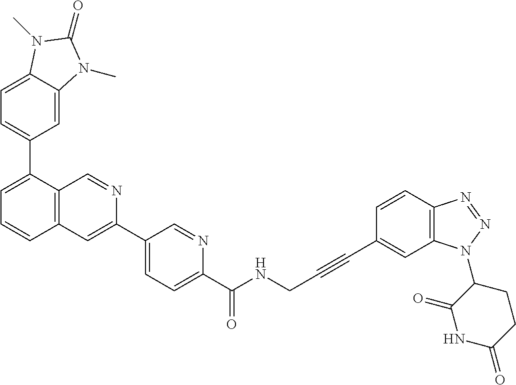 [SMILES: Cn1c(=O)n(C)c2cc(-c3cccc4cc(-c5ccc(C(=O)NCC#Cc6ccc7nnn(C8CCC(=O)NC8=O)c7c6)nc5)ncc34)ccc21] | 5-(8-(1,3-dimethyl-2- oxo-2,3-dihydro-1H- benzo[d]imidazol-5- yl)isoquinolin-3-yl)- N-(3-(1-(2,6-dioxo- piperidin-3-yl)-1H- benzo[d][1,2,3]triazol- 6-yl)prop-2-yn-1- yl)picolinamide |
| A164 | 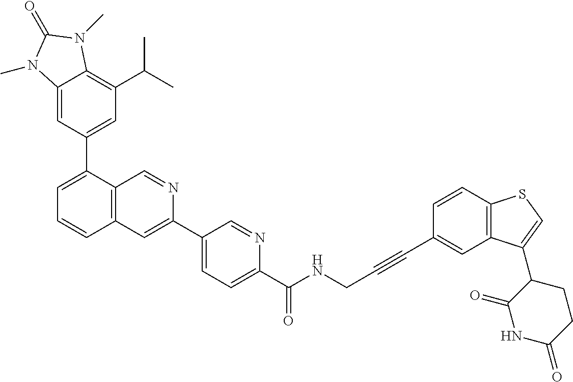 [SMILES: CC(C)c1cc(-c2cccc3cc(-c4ccc(C(=O)NCC#Cc5ccc6scc(C7CCC(=O)NC7=O)c6c5)nc4)ncc23)cc2c1n(C)c(=O)n2C] | N-(3-(3-(2,6-dioxo- piperidin-3- yl)benzo[b]thiophen- 5-yl)prop-2-yn-1-yl)- 5-(8-(7-isopropyl-1,3- dimethyl-2-oxo-2,3- dihydro-1H-benzo [d]imidazol-5- yl)isoquinolin-3- yl)picolinamide |
| A165 | 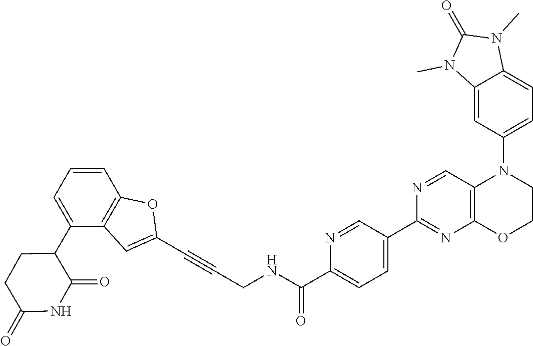 [SMILES: Cn1c(=O)n(C)c2cc(N3CCOc4nc(-c5ccc(C(=O)NCC#Cc6cc7c(C8CCC(=O)NC8=O)cccc7o6)nc5)ncc43)ccc21] | 5-(5-(1,3-dimethyl- 2-oxo-2,3-dihydro-1H- benzo[d]imidazol-5- yl)-6,7-dihydro-5H- pyrimido[4,5-b][1,4] oxazin-2-yl)-N-(3-(4- (2,6-dioxopiperidin- 3-yl)benzofuran-2- yl)prop-2-yn-1- yl)picolinamide |
| A166 | 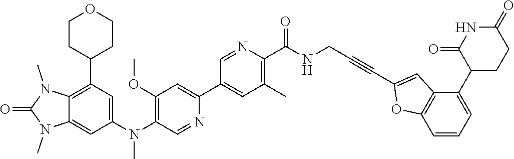 [SMILES: COc1cc(-c2cnc(C(=O)NCC#Cc3cc4c(C5CCC(=O)NC5=O)cccc4o3)c(C)c2)ncc1N(C)c1cc(C2CCOCC2)c2c(c1)n(C)c(=O)n2C] | 5-((1,3-dimethyl-2- oxo-7-(tetrahydro-2H- pyran-4-yl)-2,3- dihydro-1H-benzo[d] imidazol-5-yl)(methyl) amino)-N-(3-(4-(2,6- dioxopiperidin-3- yl)benzofuran-2-yl) prop-2-yn-1-yl)-4- methoxy-5′-methyl- |
| | | [2,3′-bipyridine]-6′- |
| | | carboxamide |
| A167 | 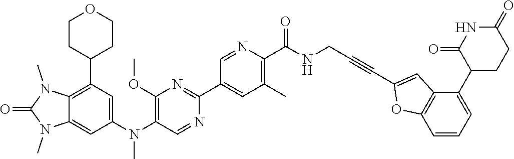 [SMILES: COc1nc(-c2cnc(C(=O)NCC#Cc3cc4c(C5CCC(=O)NC5=O)cccc4o3)c(C)c2)ncc1N(C)c1cc(C2CCOCC2)c2c(c1)n(C)c(=O)n2C] | 5-(5-((1,3-dimethyl- 2-oxo-7-(tetrahydro- 2H-pyran-4-yl)-2,3- dihydro-1H- benzo[d]imidazol-5- yl)(methyl)amino)-4- methoxypyrimidin-2- yl)-N-(3-(4-(2,6- dioxopiperidin-3- yl)benzofuran-2- |
| | | yl)prop-2-yn-1-yl)- |
| | | 3-methylpicolinamide |
| A168 | 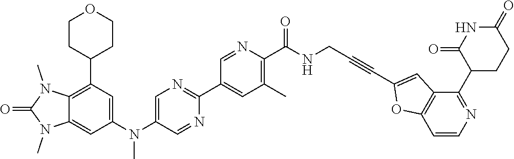 [SMILES: Cc1cc(-c2ncc(N(C)c3cc(C4CCOCC4)c4c(c3)n(C)c(=O)n4C)cn2)cnc1C(=O)NCC#Cc1cc2c(C3CCC(=O)NC3=O)nccc2o1] | 5-(5-((1,3-dimethyl-2- oxo-7-(tetrahydro- 2H-pyran-4-yl)-2,3- dihydro-1H- benzo[d]imidazol-5- yl)(methyl)amino) pyrimidin-2-yl)-N-(3- (4-(2,6-dioxopiperidin- 3-yl)furo[3,2- c]pyridin-2-yl)prop- |
| | | 2-yn-1-yl)-3- |
| | | methylpicolinamide |
| A169 | 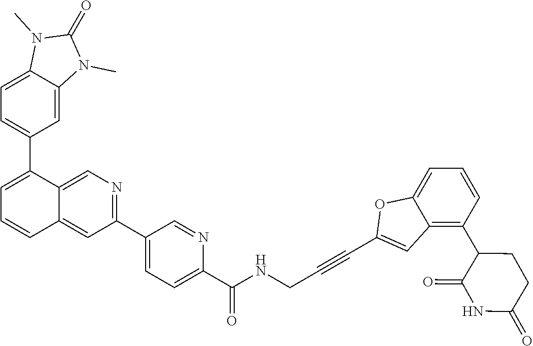 [SMILES: Cn1c(=O)n(C)c2cc(-c3cccc4cc(-c5ccc(C(=O)NCC#Cc6cc7c(C8CCC(=O)NC8=O)cccc7o6)nc5)ncc34)ccc21] | 5-(8-(1,3-dimethyl-2- oxo-2,3-dihydro-1H- benzo[d]imidazol-5- yl)isoquinolin-3-yl)- N-(3-(4-(2,6-dioxo- piperidin-3- yl)benzofuran-2- yl)prop-2-yn-1- yl)picolinamide |
| A170 | 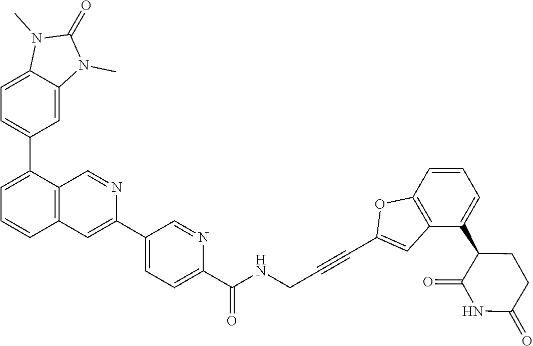 [SMILES: Cn1c(=O)n(C)c2cc(-c3cccc4cc(-c5ccc(C(=O)NCC#Cc6cc7c([C@H]8CCC(=O)NC8=O)cccc7o6)nc5)ncc34)ccc21] | (R)-5-(8-(1,3-dimethyl- 2-oxo-2,3-dihydro- 1H-benzo[d]imidazol-5- yl)isoquinolin-3-yl)- N-(3-(4-(2,6-dioxo- piperidin-3-yl)benzofuran- 2-yl)prop-2-yn-1- yl)picolinamide |
| A171 | 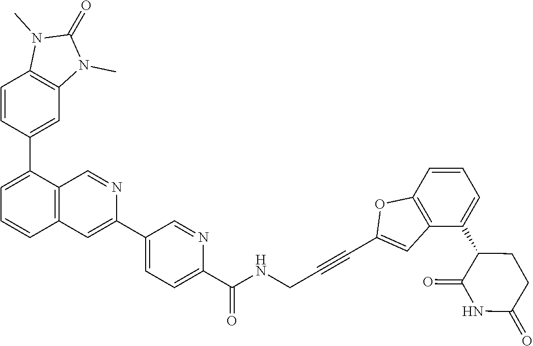 [SMILES: Cn1c(=O)n(C)c2cc(-c3cccc4cc(-c5ccc(C(=O)NCC#Cc6cc7c([C@@H]8CCC(=O)NC8=O)cccc7o6)nc5)ncc34)ccc21] | (S)-5-(8-(1,3-dimethyl- 2-oxo-2,3-dihydro- 1H-benzo[d]imidazol- 5-yl)isoquinolin-3- yl)-N-(3-(4-(2,6- dioxopiperidin-3- yl)benzofuran-2- yl)prop-2-yn-1- yl)picolinamide |
| A172 | 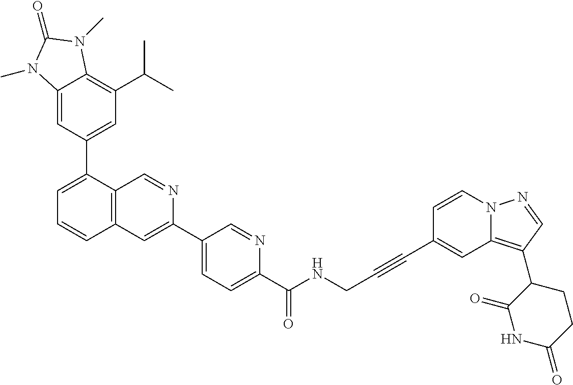 [SMILES: CC(C)c1cc(-c2cccc3cc(-c4ccc(C(=O)NCC#Cc5ccn6ncc(C7CCC(=O)NC7=O)c6c5)nc4)ncc23)cc2c1n(C)c(=O)n2C] | N-(3-(3-(2,6-dioxo- piperidin-3- yl)pyrazolo[1,5-a] pyridin-5-yl)prop-2-yn- 1-yl)-5-(8-(7-isopropyl- 1,3-dimethyl-2- oxo-2,3-dihydro-1H- benzo[d]imidazol-5- yl)isoquinolin-3- yl)picolinamide |
| A173 | 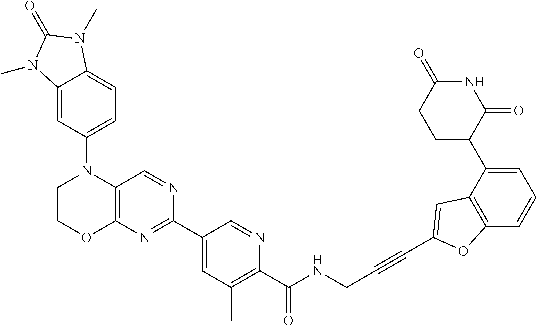 [SMILES: Cc1cc(-c2ncc3c(n2)OCCN3c2ccc3c(c2)n(C)c(=O)n3C)cnc1C(=O)NCC#Cc1cc2c(C3CCC(=O)NC3=O)cccc2o1] | 5-(5-(1,3-dimethyl-2- oxo-2,3-dihydro-1H- benzo[d]imidazol-5- yl)-6,7-dihydro-5H- pyrimido[4,5-b][1,4] oxazin-2-yl)-N-(3-(4- (2,6-dioxopiperidin- 3-yl)benzofuran-2- yl)prop-2-yn-1-yl)- 3-methylpicolinamide |
| A174 | 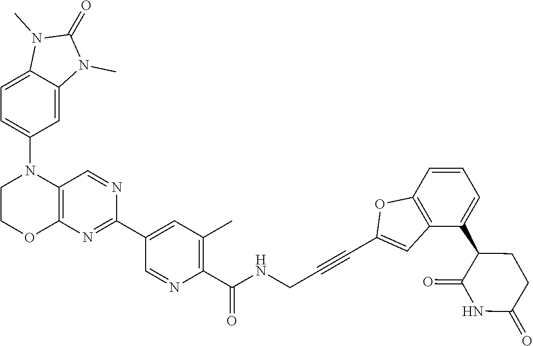 [SMILES: Cc1cc(-c2ncc3c(n2)OCCN3c2ccc3c(c2)n(C)c(=O)n3C)cnc1C(=O)NCC#Cc1cc2c([C@H]3CCC(=O)NC3=O)cccc2o1] | (R)-5-(5-(1,3-dimethyl- 2-oxo-2,3- dihydro-1H-benzo [d]imidazol-5-yl)-6,7- dihydro-5H-pyrimido [4,5-b][1,4]oxazin- 2-yl)-N-(3-(4-(2,6- dioxopiperidin-3- yl)benzofuran-2-yl) prop-2-yn-1-yl)-3- methylpicolinamide |
| A175 | 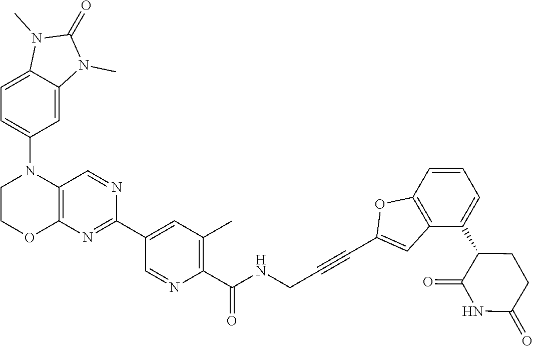 [SMILES: Cc1cc(-c2ncc3c(n2)OCCN3c2ccc3c(c2)n(C)c(=O)n3C)cnc1C(=O)NCC#Cc1cc2c([C@@H]3CCC(=O)NC3=O)cccc2o1] | (S)-5-(5-(1,3-dimethyl- 2-oxo-2,3-dihydro- 1H-benzo[d]imidazol- 5-yl)-6,7-dihydro- 5H-pyrimido[4,5-b] [1,4]oxazin-2-yl)-N- (3-(4-(2,6-dioxo- piperidin-3- yl)benzofuran-2-yl) prop-2-yn-1-yl)-3- methylpicolinamide |
| A176 | 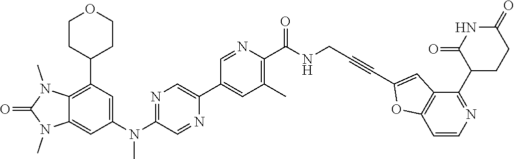 [SMILES: Cc1cc(-c2cnc(N(C)c3cc(C4CCOCC4)c4c(c3)n(C)c(=O)n4C)cn2)cnc1C(=O)NCC#Cc1cc2c(C3CCC(=O)NC3=O)nccc2o1] | 5-(5-((1,3-dimethyl- 2-oxo-7-(tetrahydro- 2H-pyran-4-yl)-2,3- dihydro-1H- benzo[d]imidazol-5- yl)(methyl)amino) pyrazin-2-yl)-N-(3-(4- (2,6-dioxopiperidin- 3-yl)furo[3,2-c]pyridin- 2-yl)prop-2-yn-1-yl)-3- |
| | | methylpicolinamide |
| A177 | 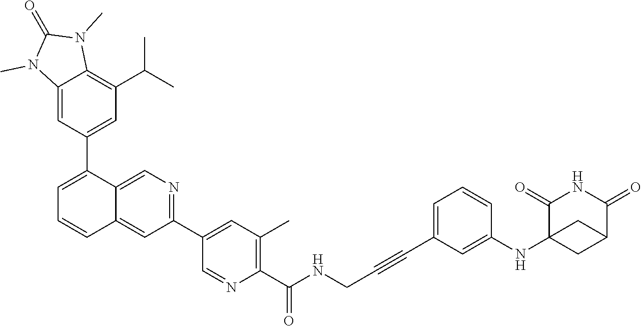 [SMILES: Cc1cc(-c2cc3cccc(-c4cc(C(C)C)c5c(c4)n(C)c(=O)n5C)c3cn2)cnc1C(=O)NCC#Cc1cccc(NC23CC(C2)C(=O)NC3=O)c1] | N-(3-(3-((2,4-dioxo-3- azabicyclo[3.1.1]heptan-1- yl)amino)phenyl)prop- 2-yn-1-yl)-5-(8-(7- isopropyl-1,3-dimethyl- 2-oxo-2,3-dihydro- 1H-benzo[d]imidazol-5- yl)isoquinolin-3-yl)-3- methylpicolinamide |
| A178 | 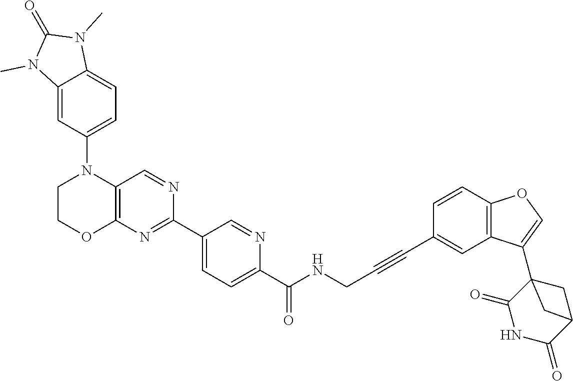 [SMILES: Cn1c(=O)n(C)c2cc(N3CCOc4nc(-c5ccc(C(=O)NCC#Cc6ccc7occ(C89CC(C8)C(=O)NC9=O)c7c6)nc5)ncc43)ccc21] | 5-(5-(1,3-dimethyl-2- oxo-2,3-dihydro-1H- benzo[d]imidazol-5- yl)-6,7-dihydro-5H- pyrimido[4,5-b][1,4] oxazin-2-yl)-N-(3-(3- (2,4-dioxo-3-azabicyclo [3.1.1]heptan-1- yl)benzofuran-5- yl)prop-2-yn-1- yl)picolinamide |
| A179 | 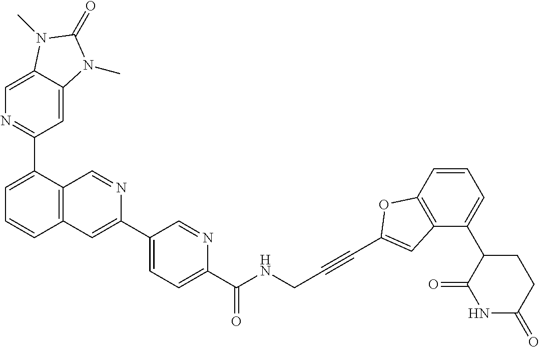 [SMILES: Cn1c(=O)n(C)c2cc(-c3cccc4cc(-c5ccc(C(=O)NCC#Cc6cc7c(C8CCC(=O)NC8=O)cccc7o6)nc5)ncc34)ncc21] | 5-(8-(1,3-dimethyl-2- oxo-2,3-dihydro-1H- imidazo[4,5-c]pyridin- 6-yl)isoquinolin-3- yl)-N-(3-(4-(2,6- dioxopiperidin-3- yl)benzofuran-2- yl)prop-2-yn-1- yl)picolinamide |
| A180 | 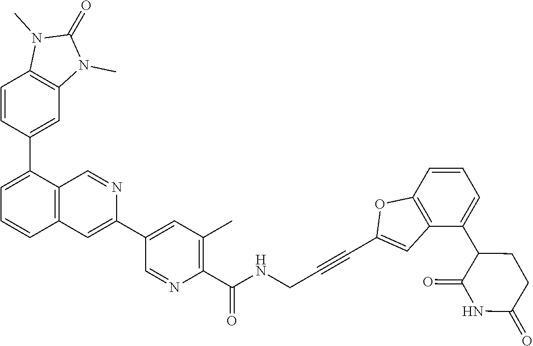 [SMILES: Cc1cc(-c2cc3cccc(-c4ccc5c(c4)n(C)c(=O)n5C)c3cn2)cnc1C(=O)NCC#Cc1cc2c(C3CCC(=O)NC3=O)cccc2o1] | 5-(8-(1,3-dimethyl-2- oxo-2,3-dihydro-1H- benzo[d]imidazol-5- yl)isoquinolin-3-yl)- N-(3-(4-(2,6-dioxo- piperidin-3- yl)benzofuran-2-yl) prop-2-yn-1-yl)-3- methylpicolinamide |
| A181 | 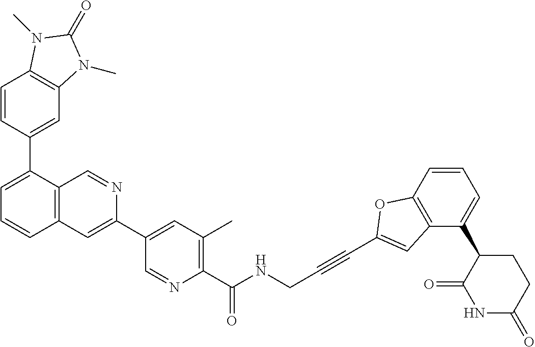 [SMILES: Cc1cc(-c2cc3cccc(-c4ccc5c(c4)n(C)c(=O)n5C)c3cn2)cnc1C(=O)NCC#Cc1cc2c([C@H]3CCC(=O)NC3=O)cccc2o1] | (R)-5-(8-(1,3-dimethyl- 2-oxo-2,3- dihydro-1H-benzo [d]imidazol-5- yl)isoquinolin-3-yl)- N-(3-(4-(2,6- dioxopiperidin-3-yl) benzofuran-2- yl)prop-2-yn-1-yl)- 3-methylpicolinamide |
| A182 | 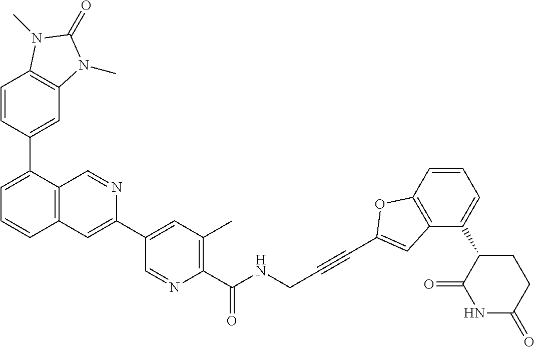 [SMILES: Cc1cc(-c2cc3cccc(-c4ccc5c(c4)n(C)c(=O)n5C)c3cn2)cnc1C(=O)NCC#Cc1cc2c([C@@H]3CCC(=O)NC3=O)cccc2o1] | (S)-5-(8-(1,3-dimethyl- 2-oxo-2,3-dihydro- 1H-benzo[d]imidazol- 5-yl)isoquinolin-3- yl)-N-(3-(4-(2,6- dioxopiperidin-3- yl)benzofuran-2- yl)prop-2-yn-1-yl)-3- methylpicolinamide |
| A183 | 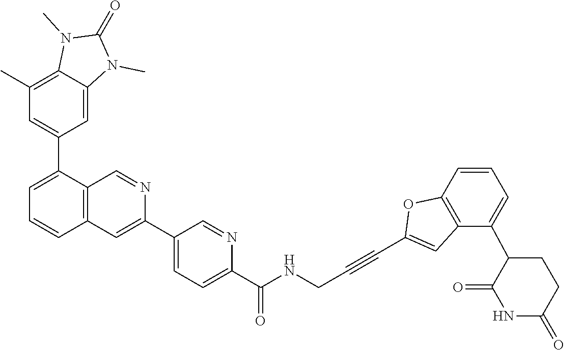 [SMILES: Cc1cc(-c2cccc3cc(-c4ccc(C(=O)NCC#Cc5cc6c(C7CCC(=O)NC7=O)cccc6o5)nc4)ncc23)cc2c1n(C)c(=O)n2C] | N-(3-(4-(2,6-dioxo- piperidin-3- yl)benzofuran-2-yl) prop-2-yn-1-yl)-5-(8- (1,3,7-trimethyl-2- oxo-2,3-dihydro-1H- benzo[d]imidazol- 5-yl)isoquinolin-3- yl)picolinamide |
| A184 | 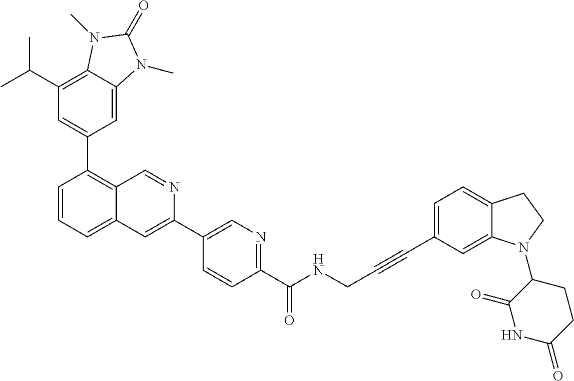 [SMILES: CC(C)c1cc(-c2cccc3cc(-c4ccc(C(=O)NCC#Cc5ccc6c(c5)N(C5CCC(=O)NC5=O)CC6)nc4)ncc23)cc2c1n(C)c(=O)n2C] | N-(3-(1-(2,6-dioxo- piperidin-3-yl)indolin- 6-yl)prop-2-yn-1- yl)-5-(8-(7-isopropyl- 1,3-dimethyl-2-oxo- 2,3-dihydro-1H- benzo[d]imidazol- 5-yl)isoquinolin-3- yl)picolinamide |
| A185 | 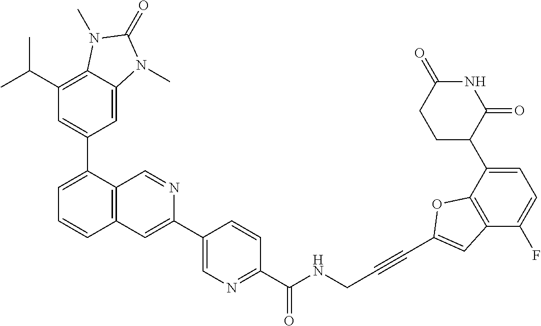 [SMILES: CC(C)c1cc(-c2cccc3cc(-c4ccc(C(=O)NCC#Cc5cc6c(F)ccc(C7CCC(=O)NC7=O)c6o5)nc4)ncc23)cc2c1n(C)c(=O)n2C] | N-(3-(7-(2,6-dioxo- piperidin-3-yl)-4- fluorobenzofuran-2- yl)prop-2-yn-1-yl)-5- (8-(7-isopropyl-1,3- dimethyl-2-oxo-2,3- dihydro-1H-benzo [d]imidazol-5- yl)isoquinolin-3- yl)picolinamide |
| A186 | 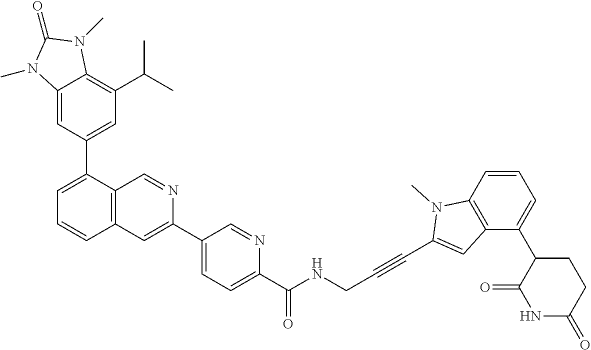 [SMILES: CC(C)c1cc(-c2cccc3cc(-c4ccc(C(=O)NCC#Cc5cc6c(C7CCC(=O)NC7=O)cccc6n5C)nc4)ncc23)cc2c1n(C)c(=O)n2C] | N-(3-(4-(2,6-dioxo- piperidin-3-yl)-1- methyl-1H-benzo[d] imidazol-2-yl)prop-2- yn-1-yl)-5-(8-(7-iso- propyl-1,3-dimethyl- 2-oxo-2,3-dihydro- 1H-benzo[d]imidazol- 5-yl)isoquinolin-3- yl)picolinamide |
| A187 | 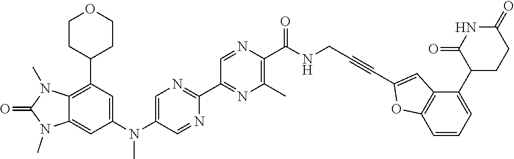 [SMILES: Cc1nc(-c2ncc(N(C)c3cc(C4CCOCC4)c4c(c3)n(C)c(=O)n4C)cn2)cnc1C(=O)NCC#Cc1cc2c(C3CCC(=O)NC3=O)cccc2o1] | 5-(5-((1,3-dimethyl- 2-oxo-7-(tetrahydro- 2H-pyran-4-yl)-2,3- dihydro-1H- benzo[d]imidazol-5- yl)(methyl)amino) pyrimidin-2-yl)-N-(3- (4-(2,6-dioxopiperidin- 3-yl)benzofuran-2- yl)prop-2-yn-1-yl)- |
| | | 3-methylpyrazine-2- |
| | | carboxamide |
| A188 | 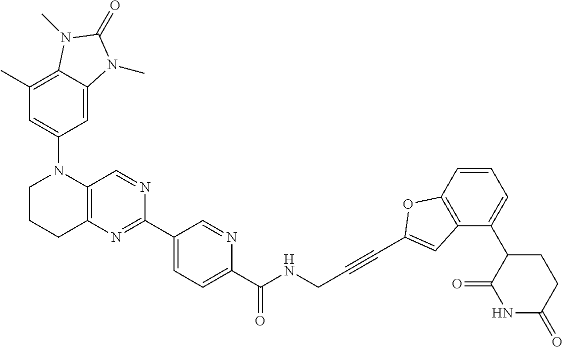 [SMILES: Cc1cc(N2CCCc3nc(-c4ccc(C(=O)NCC#Cc5cc6c(C7CCC(=O)NC7=O)cccc6o5)nc4)ncc32)cc2c1n(C)c(=O)n2C] | N-(3-(4-(2,6-dioxo- piperidin-3- yl)benzofuran-2-yl) prop-2-yn-1-yl)-5-(5- (1,3,7-trimethyl-2- oxo-2,3-dihydro-1H- benzo[d]imidazol- 5-yl)-5,6,7,8- tetrahydropyrido[3,2- d]pyrimidin-2- yl)picolinamide |
| A189 | 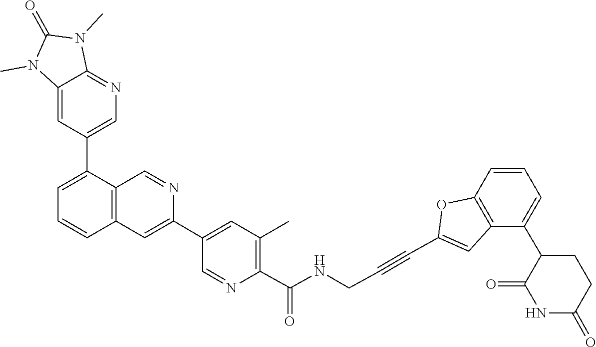 [SMILES: Cc1cc(-c2cc3cccc(-c4cnc5c(c4)n(C)c(=O)n5C)c3cn2)cnc1C(=O)NCC#Cc1cc2c(C3CCC(=O)NC3=O)cccc2o1] | 5-(8-(1,3-dimethyl-2- oxo-2,3-dihydro-1H- imidazo[4,5-b]pyridin- 6-yl)isoquinolin-3- yl)-N-(3-(4-(2,6- dioxopiperidin-3- yl)benzofuran-2-yl) prop-2-yn-1-yl)-3- methylpicolinamide |
| A190 | 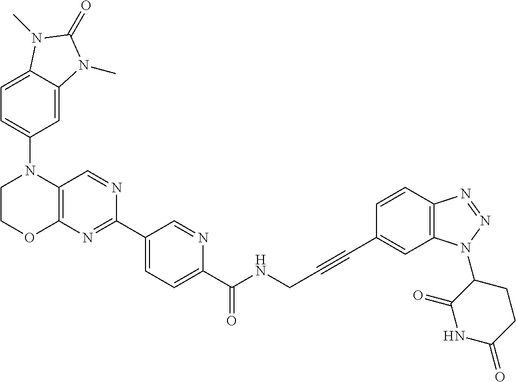 [SMILES: Cn1c(=O)n(C)c2cc(N3CCOc4nc(-c5ccc(C(=O)NCC#Cc6ccc7nnn(C8CCC(=O)NC8=O)c7c6)nc5)ncc43)ccc21] | 5-(5-(1,3-dimethyl-2- oxo-2,3-dihydro-1H- benzo[d]imidazol-5- yl)-6,7-dihydro-5H- pyrimido[4,5-b][1,4] oxazin-2-yl)-N-(3-(1- (2,6-dioxopiperidin- 3-yl)-1H-benzo[d] [1,2,3]triazol-6-yl) prop-2-yn-1- yl)picolinamide |
| A191 | 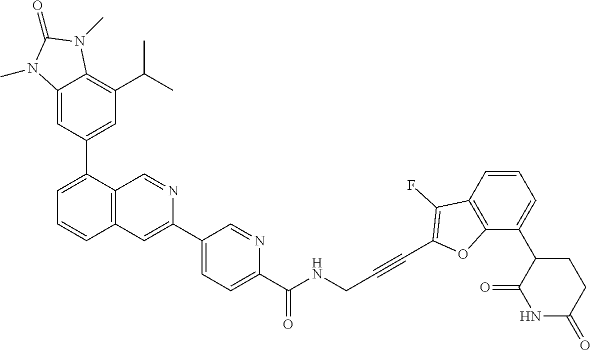 [SMILES: CC(C)c1cc(-c2cccc3cc(-c4ccc(C(=O)NCC#Cc5oc6c(C7CCC(=O)NC7=O)cccc6c5F)nc4)ncc23)cc2c1n(C)c(=O)n2C] | N-(3-(7-(2,6-dioxo- piperidin-3-yl)-3- fluorobenzofuran-2- yl)prop-2-yn-1-yl)-5- (8-(7-isopropyl-1,3- dimethyl-2-oxo-2,3- dihydro-1H-benzo [d]imidazol-5- yl)isoquinolin-3- yl)picolinamide |
| A192 | 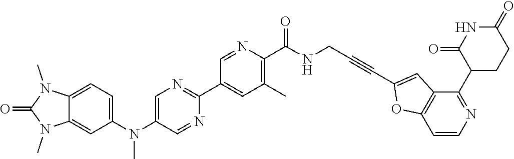 [SMILES: Cc1cc(-c2ncc(N(C)c3ccc4c(c3)n(C)c(=O)n4C)cn2)cnc1C(=O)NCC#Cc1cc2c(C3CCC(=O)NC3=O)nccc2o1] | 5-(5-((1,3-dimethyl- 2-oxo-2,3-dihydro- 1H-benzo[d]imidazol-5- yl)(methyl)amino) pyrimidin-2-yl)-N-(3- (4-(2,6-dioxopiperidin- 3-yl)furo[3,2- c]pyridin-2-yl)prop- 2-yn-1-yl)-3- methylpicolinamide |
| A193 | 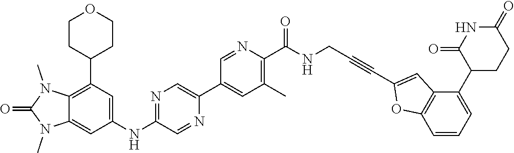 [SMILES: Cc1cc(-c2cnc(Nc3cc(C4CCOCC4)c4c(c3)n(C)c(=O)n4C)cn2)cnc1C(=O)NCC#Cc1cc2c(C3CCC(=O)NC3=O)cccc2o1] | 5-(5-(1,3-dimethyl- 2-oxo-7-(tetrahydro- 2H-pyran-4-yl)- 2,3-dihydro-1H- benzo[d]imidazol- 5-yl)amino)pyrazin-2- yl)-N-(3-(4-(2,6- dioxopiperidin-3- yl)benzofuran-2-yl) prop-2-yn-1-yl)-3- |
| | | methylpicolinamide |
| A194 | 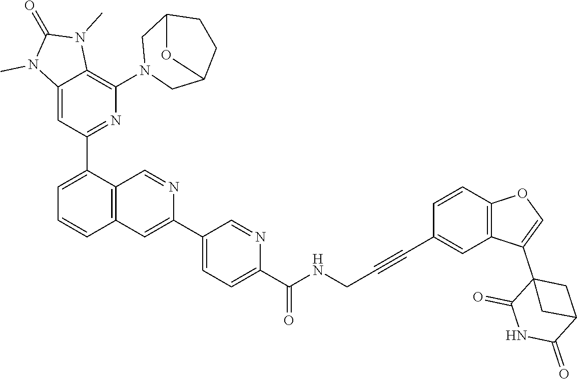 [SMILES: Cn1c(=O)n(C)c2c(N3CC4CCC(C3)O4)nc(-c3cccc4cc(-c5ccc(C(=O)NCC#Cc6ccc7occ(C89CC(C8)C(=O)NC9=O)c7c6)nc5)ncc34)cc21] | 5-(8-(4-(8-oxa-3- azabicyclo[3.2.1]octan- 3-yl)-1,3-dimethyl-2- oxo-2,3-dihydro-1H- imidazo[4,5-c]pyridin- 6-yl)isoquinolin-3- yl)-N-(3-(3-(2,4-dioxo- 3-azabicyclo[3.1.1] heptan-1-yl)benzofuran- 5-yl)prop-2-yn-1- yl)picolinamide |
| A195 | 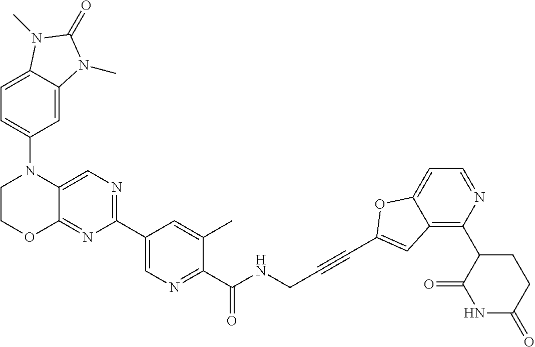 [SMILES: Cc1cc(-c2ncc3c(n2)OCCN3c2ccc3c(c2)n(C)c(=O)n3C)cnc1C(=O)NCC#Cc1cc2c(C3CCC(=O)NC3=O)nccc2o1] | 5-(5-(1,3-dimethyl-2- oxo-2,3-dihydro-1H- benzo[d]imidazol-5- yl)-6,7-dihydro-5H- pyrimido[4,5-b][1,4] oxazin-2-yl)-N-(3-(4- (2,6-dioxopiperidin- 3-yl)furo[3,2- c]pyridin-2-yl)prop- 2-yn-1-yl)-3- methylpicolinamide |
| A196 | 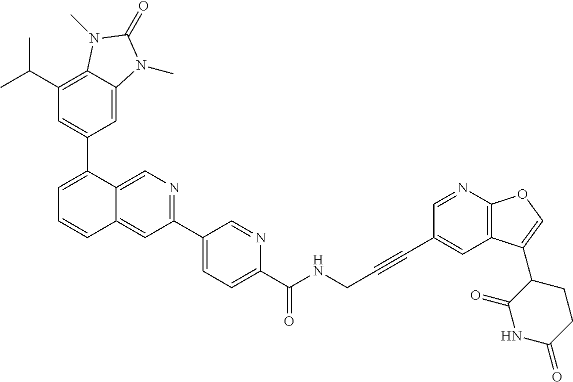 [SMILES: CC(C)c1cc(-c2cccc3cc(-c4ccc(C(=O)NCC#Cc5cnc6occ(C7CCC(=O)NC7=O)c6c5)nc4)ncc23)cc2c1n(C)c(=O)n2C] | N-(3-(3-(2,6-dioxo- piperidin-3-yl)furo[2,3- b]pyridin-5-yl)prop- 2-yn-1-yl)-5-(8-(7- isopropyl-1,3-dimethyl- 2-oxo-2,3-dihydro- 1H-benzo[d]imidazol- 5-yl)isoquinolin-3- yl)picolinamide |
| A197 | 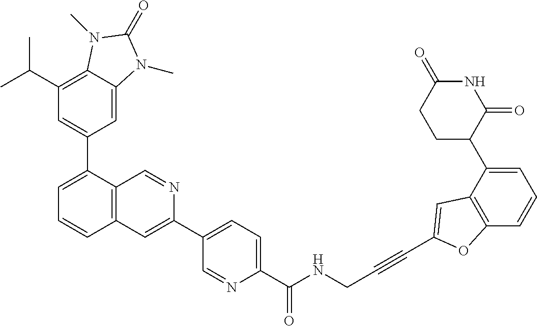 [SMILES: CC(C)c1cc(-c2cccc3cc(-c4ccc(C(=O)NCC#Cc5cc6c(C7CCC(=O)NC7=O)cccc6o5)nc4)ncc23)cc2c1n(C)c(=O)n2C] | N-(3-(4-(2,6-dioxo- piperidin-3- yl)benzofuran-2-yl) prop-2-yn-1-yl)-5-(8- (7-isopropyl-1,3- dimethyl-2-oxo-2,3- dihydro-1H-benzo [d]imidazol-5- yl)isoquinolin-3- yl)picolinamide |
| A198 | 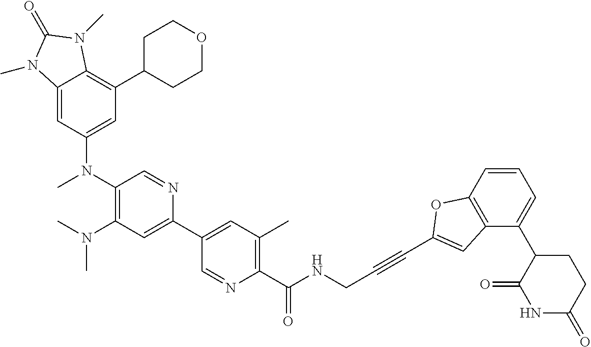 [SMILES: Cc1cc(-c2cc(N(C)C)c(N(C)c3cc(C4CCOCC4)c4c(c3)n(C)c(=O)n4C)cn2)cnc1C(=O)NCC#Cc1cc2c(C3CCC(=O)NC3=O)cccc2o1] | 5-((1,3-dimethyl-2- oxo-7-(tetrahydro-2H- pyran-4-yl)-2,3- dihydro-1H- benzo[d]imidazol-5- yl)(methyl)amino)-4- (dimethylamino)- N-(3-(4-(2,6- dioxopiperidin-3- yl)benzofuran-2- yl)prop-2-yn-1- yl)-5′-methyl-[2,3′- bipyridine]-6′- carboxamide |
| A199 | 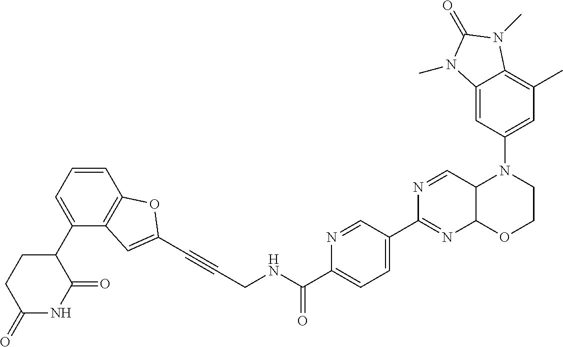 [SMILES: Cc1cc(N2CCOC3N=C(c4ccc(C(=O)NCC#Cc5cc6c(C7CCC(=O)NC7=O)cccc6o5)nc4)N=CC32)cc2c1n(C)c(=O)n2C] | N-(3-(4-(2,6-dioxo- piperidin-3- yl)benzofuran-2-yl) prop-2-yn-1-yl)-5-(5- (1,3,7-trimethyl-2- oxo-2,3-dihydro-1H- benzo[d]imidazol-5- yl)-6,7-dihydro-5H- pyrimido[4,5-b] [1,4]oxazin-2- yl)picolinamide |
| A200 | 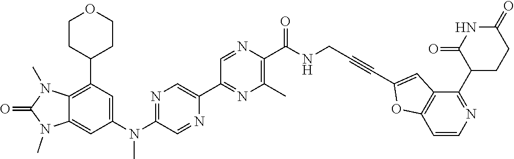 [SMILES: Cc1nc(-c2cnc(N(C)c3cc(C4CCOCC4)c4c(c3)n(C)c(=O)n4C)cn2)cnc1C(=O)NCC#Cc1cc2c(C3CCC(=O)NC3=O)nccc2o1] | 5′-((1,3-dimethyl-2- oxo-7-(tetrahydro-2H- pyran-4-yl)-2,3- dihydro-1H-benzo [d]imidazol-5-yl) (methyl)amino)-N- (3-(4-(2,6-dioxo- piperidin-3-yl)furo[3,2- c]pyridin-2-yl)prop-2- yn-1-yl)-6-methyl- |
| | | [2,2′-bipyrazine]-5- |
| | | carboxamide |
| A201 | 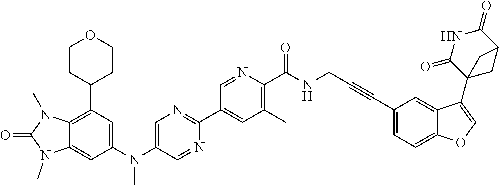 [SMILES: Cc1cc(-c2ncc(N(C)c3cc(C4CCOCC4)c4c(c3)n(C)c(=O)n4C)cn2)cnc1C(=O)NCC#Cc1ccc2occ(C34CC(C3)C(=O)NC4=O)c2c1] | 5-(5-((1,3-dimethyl- 2-oxo-7-(tetrahydro- 2H-pyran-4-yl)-2,3- dihydro-1H- benzo[d]imidazol-5- yl)(methyl)amino) pyrimidin-2-yl)-N-(3- (3-(2,4-dioxo-3- azabicyclo[3.1.1]heptan- 1-yl)benzofuran-5-yl) prop-2-yn-1-yl)-3- methylpicolinamide |
| A202 | 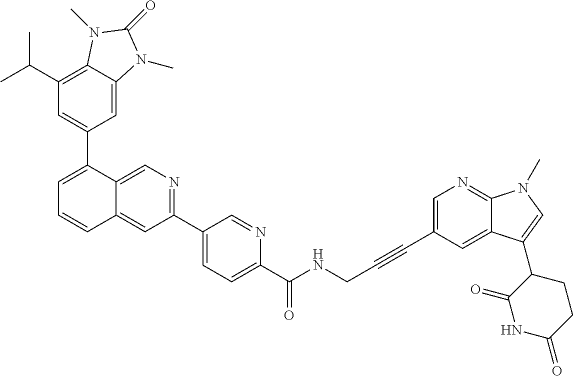 [SMILES: CC(C)c1cc(-c2cccc3cc(-c4ccc(C(=O)NCC#Cc5cnc6c(c5)c(C5CCC(=O)NC5=O)cn6C)nc4)ncc23)cc2c1n(C)c(=O)n2C] | N-(3-(3-(2,6-dioxo- piperidin-3-yl)-1- methyl-1H-pyrrolo [2,3-b]pyridin-5- yl)prop-2-yn-1-yl)- 5-(8-(7-isopropyl-1,3- dimethyl-2-oxo-2,3- dihydro-1H- benzo[d]imidazol-5- yl)isoquinolin-3- yl)picolinamide |
| A203 | 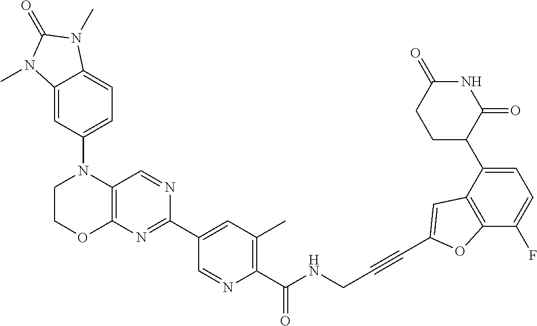 [SMILES: Cc1cc(-c2ncc3c(n2)OCCN3c2ccc3c(c2)n(C)c(=O)n3C)cnc1C(=O)NCC#Cc1cc2c(C3CCC(=O)NC3=O)ccc(F)c2o1] | 5-(5-(1,3-dimethyl-2- oxo-2,3-dihydro-1H- benzo[d]imidazol-5- yl)-6,7-dihydro-5H- pyrimido[4,5-b][1,4] oxazin-2-yl)-N-(3-(4- (2,6-dioxopiperidin- 3-yl)-7-fluoro- benzofuran-2-yl)prop- 2-yn-1-yl)-3- methylpicolinamide |
| A204 | 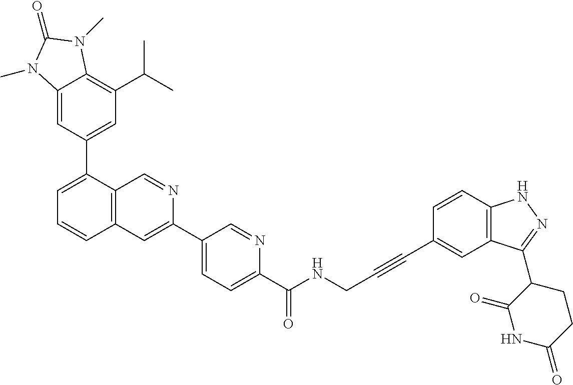 [SMILES: CC(C)c1cc(-c2cccc3cc(-c4ccc(C(=O)NCC#Cc5ccc6[nH]nc(C7CCC(=O)NC7=O)c6c5)nc4)ncc23)cc2c1n(C)c(=O)n2C] | N-(3-(3-(2,6-dioxo- piperidin-3-yl)-1H- indazol-5-yl)prop-2- yn-1-yl)-5-(8-(7- isopropyl-1,3- dimethyl-2-oxo-2,3- dihydro-1H-benzo [d]imidazol-5- yl)isoquinolin-3- yl)picolinamide |
| A205 | 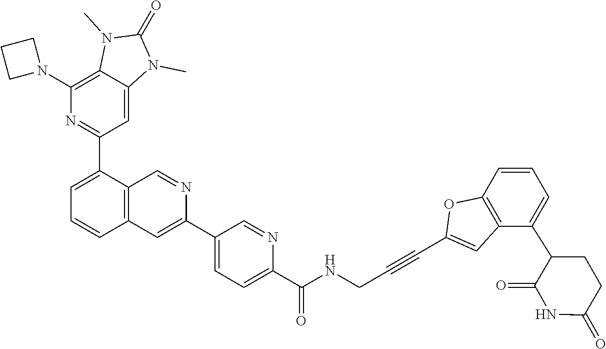 [SMILES: Cn1c(=O)n(C)c2c(N3CCC3)nc(-c3cccc4cc(-c5ccc(C(=O)NCC#Cc6cc7c(C8CCC(=O)NC8=O)cccc7o6)nc5)ncc34)cc21] | 5-(8-(4-(azetidin-1- yl)-1,3-dimethyl-2- oxo-2,3-dihydro- 1H-imidazo[4,5- c]pyridin-6-yl)iso- quinolin-3-yl)-N-(3-(4- (2,6-dioxopiperidin-3- yl)benzofuran-2- yl)prop-2-yn-1- yl)picolinamide |
| A206 | 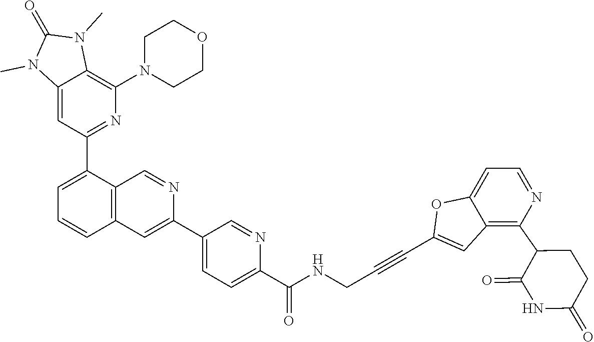 [SMILES: Cn1c(=O)n(C)c2c(N3CCOCC3)nc(-c3cccc4cc(-c5ccc(C(=O)NCC#Cc6cc7c(C8CCC(=O)NC8=O)nccc7o6)nc5)ncc34)cc21] | 5-(8-(1,3-dimethyl-4- morpholino-2-oxo- 2,3-dihydro-1H- imidazo[4,5-c]pyridin-6- yl)isoquinolin-3-yl)- N-(3-(4-(2,6- dioxopiperidin-3-yl) furo[3,2-c]pyridin-2- yl)prop-2-yn-1- yl)picolinamide |
| A207 | 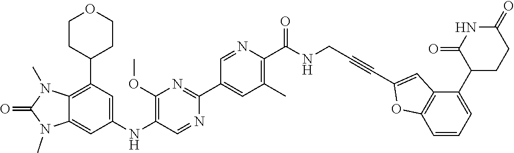 [SMILES: COc1nc(-c2cnc(C(=O)NCC#Cc3cc4c(C5CCC(=O)NC5=O)cccc4o3)c(C)c2)ncc1Nc1cc(C2CCOCC2)c2c(c1)n(C)c(=O)n2C] | 5-(5-((1,3-dimethyl- 2-oxo-7-(tetrahydro- 2H-pyran-4-yl)-2,3- dihydro-1H- benzo[d]imidazol-5- yl)amino)-4- methoxypyrimidin-2- yl)-N-(3-(4-(2,6- dioxopiperidin-3- yl)benzofuran-2- |
| | | yl)prop-2-yn-1-yl)- |
| | | 3-methylpicolinamide |
| A208 | 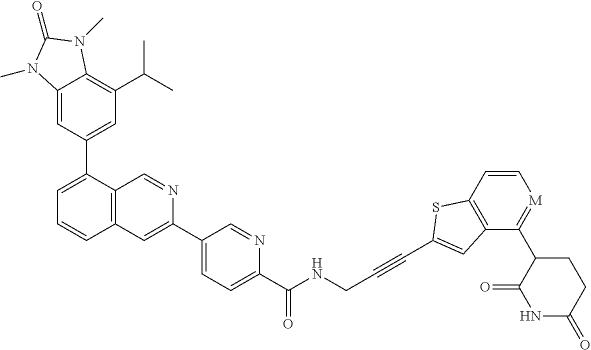 | N-(3-(4-(2,6-dioxo- piperidin-3- yl)thieno[3,2-c]pyridin- 2-yl)prop-2-yn-1- yl)-5-(8-(7-isopropyl- 1,3-dimethyl-2-oxo- 2,3-dihydro-1H- benzo[d]imidazol-5- yl)isoquinolin-3- yl)picolinamide |
| A209 | 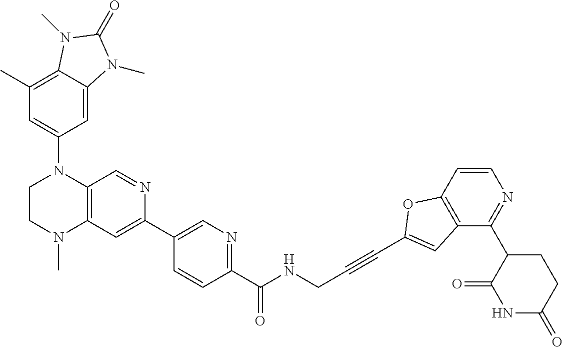 [SMILES: Cc1cc(N2CCN(C)c3cc(-c4ccc(C(=O)NCC#Cc5cc6c(C7CCC(=O)NC7=O)nccc6o5)nc4)ncc32)cc2c1n(C)c(=O)n2C] | N-(3-(4-(2,6-dioxo- piperidin-3-yl)furo [3,2-c]pyridin-2- yl)prop-2-yn-1-yl)- 5-(1-methyl-4- (1,3,7-trimethyl-2- oxo-2,3-dihydro- 1H-benzo[d]imidazol- 5-yl)-1,2,3,4- tetrahydropyrido[3,4- b]pyrazin-7- yl)picolinamide |
| A210 | 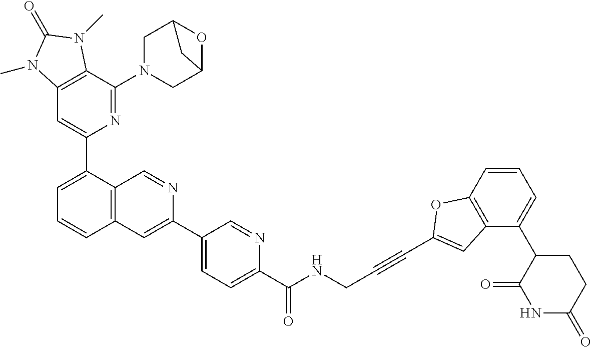 [SMILES: Cn1c(=O)n(C)c2c(N3CC4CC(C3)O4)nc(-c3cccc4cc(-c5ccc(C(=O)NCC#Cc6cc7c(C8CCC(=O)NC8=O)cccc7o6)nc5)ncc34)cc21] | 5-(8-(4-(6-oxa-3- azabicyclo[3.1.1]heptan- 3-yl)-1,3-dimethyl-2- oxo-2,3-dihydro-1H- imidazo[4,5-c]pyridin- 6-yl)isoquinolin-3- yl)-N-(3-(4-(2,6- dioxopiperidin-3- yl)benzofuran-2- yl)prop-2-yn-1- yl)picolinamide |
| A211 | 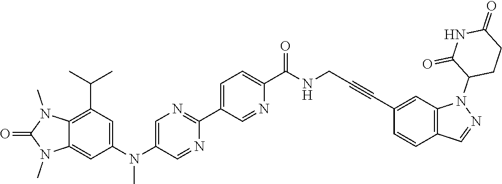 [SMILES: CC(C)c1cc(N(C)c2cnc(-c3ccc(C(=O)NCC#Cc4ccc5cnn(C6CCC(=O)NC6=O)c5c4)nc3)nc2)cc2c1n(C)c(=O)n2C] | N-(3-(1-(2,6-dioxo- piperidin-3-yl)-1H- indazol-6-yl)prop-2- yn-1-yl)-5-(5-((7- isopropyl-1,3- dimethyl-2-oxo-2,3- dihydro-1H-benzo [d]imidazol-5- yl)(methyl)amino) pyrimidin-2- yl)picolinamide |
| A212 | 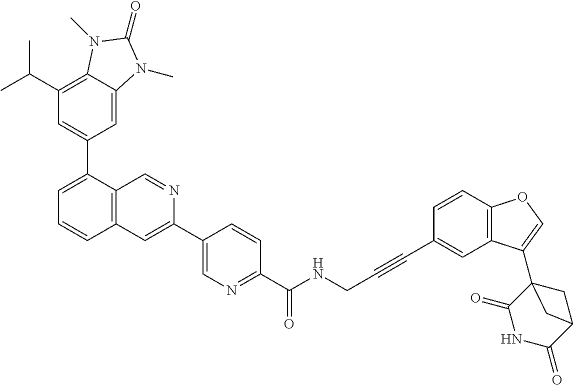 [SMILES: CC(C)c1cc(-c2cccc3cc(-c4ccc(C(=O)NCC#Cc5ccc6occ(C78CC(C7)C(=O)NC8=O)c6c5)nc4)ncc23)cc2c1n(C)c(=O)n2C] | N-(3-(3-(2,4-dioxo-3- azabicyclo[3.1.1]heptan- 1-yl)benzofuran- 5-yl)prop-2-yn-1-yl)- 5-(8-(7-isopropyl- 1,3-dimethyl-2-oxo- 2,3-dihydro-1H- benzo[d]imidazol- 5-yl)isoquinolin-3- yl)picolinamide |
| A213 | 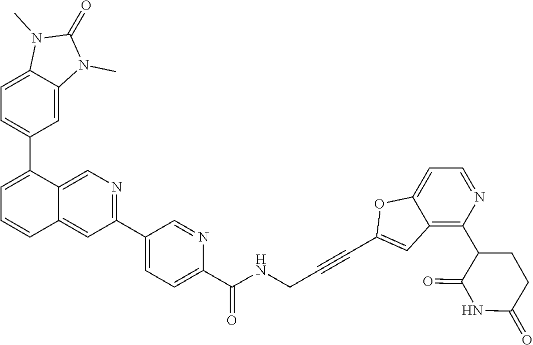 [SMILES: Cn1c(=O)n(C)c2cc(-c3cccc4cc(-c5ccc(C(=O)NCC#Cc6cc7c(C8CCC(=O)NC8=O)nccc7o6)nc5)ncc34)ccc21] | 5-(8-(1,3-dimethyl-2- oxo-2,3-dihydro-1H- benzo[d]imidazol-5- yl)isoquinolin-3-yl)- N-(3-(4-(2,6-dioxo- piperidin-3-yl)furo[3,2- c]pyridin-2-yl) prop-2-yn-1- yl)picolinamide |
| A214 | 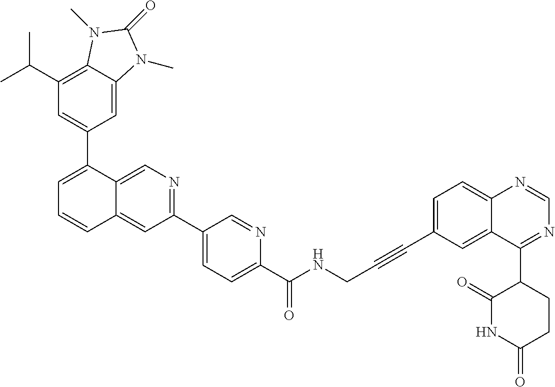 [SMILES: CC(C)c1cc(-c2cccc3cc(-c4ccc(C(=O)NCC#Cc5ccc6ncnc(C7CCC(=O)NC7=O)c6c5)nc4)ncc23)cc2c1n(C)c(=O)n2C] | N-(3-(4-(2,6-dioxo- piperidin-3- yl)quinazolin-6-yl) prop-2-yn-1-yl)-5-(8- (7-isopropyl-1,3- dimethyl-2-oxo-2,3- dihydro-1H-benzo [d]imidazol-5- yl)isoquinolin-3- yl)picolinamide |
| A215 | 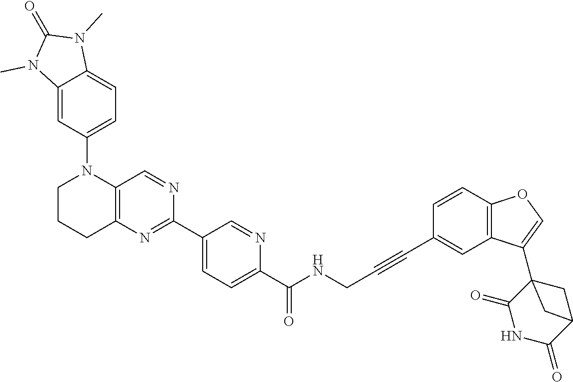 [SMILES: Cn1c(=O)n(C)c2cc(N3CCCc4nc(-c5ccc(C(=O)NCC#Cc6ccc7occ(C89CC(C8)C(=O)NC9=O)c7c6)nc5)ncc43)ccc21] | 5-(5-(1,3-dimethyl-2- oxo-2,3-dihydro-1H- benzo[d]imidazol- 5-yl)-5,6,7,8- tetrahydropyrido[3,2- d]pyrimidin-2-yl)- N-(3-(3-(2,4-dioxo-3- azabicyclo[3.1.1] heptan-1-yl)benzofuran- 5-yl)prop-2-yn-1- yl)picolinamide |
| A216 | 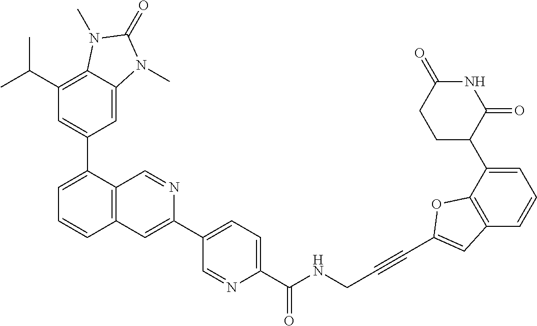 [SMILES: CC(C)c1cc(-c2cccc3cc(-c4ccc(C(=O)NCC#Cc5cc6cccc(C7CCC(=O)NC7=O)c6o5)nc4)ncc23)cc2c1n(C)c(=O)n2C] | N-(3-(7-(2,6-dioxo- piperidin-3- yl)benzofuran-2-yl) prop-2-yn-1-yl)-5-(8- (7-isopropyl-1,3- dimethyl-2-oxo-2,3- dihydro-1H-benzo [d]imidazol-5- yl)isoquinolin-3- yl)picolinamide |
| A217 | 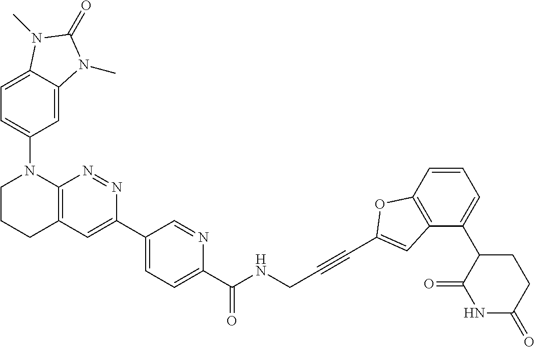 [SMILES: Cn1c(=O)n(C)c2cc(N3CCCc4cc(-c5ccc(C(=O)NCC#Cc6cc7c(C8CCC(=O)NC8=O)cccc7o6)nc5)nnc43)ccc21] | 5-(8-(1,3-dimethyl-2- oxo-2,3-dihydro-1H- benzo[d]imidazol- 5-yl)-5,6,7,8- tetrahydropyrido[2,3- c]pyridazin-3-yl)-N- (3-(4-(2,6-dioxo- piperidin-3- yl)benzofuran-2- yl)prop-2-yn-1- yl)picolinamide |
| A218 | 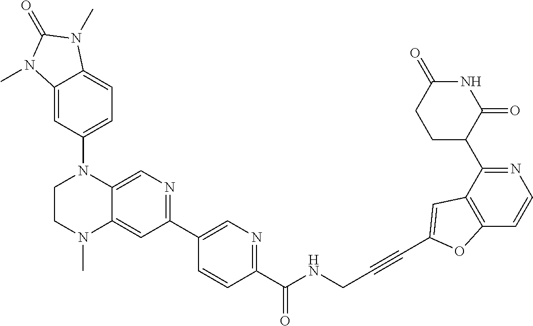 [SMILES: CN1CCN(c2ccc3c(c2)n(C)c(=O)n3C)c2cnc(-c3ccc(C(=O)NCC#Cc4cc5c(C6CCC(=O)NC6=O)nccc5o4)nc3)cc21] | 5-(4-(1,3-dimethyl-2- oxo-2,3-dihydro-1H- benzo[d]imidazol-5- yl)-1-methyl-1,2,3,4- tetrahydropyrido[3,4- b]pyrazin-7-yl)-N- (3-(4-(2,6-dioxo- piperidin-3-yl)furo[3,2- c]pyridin-2-yl)prop- 2-yn-1-yl)picolinamide |
| A219 | 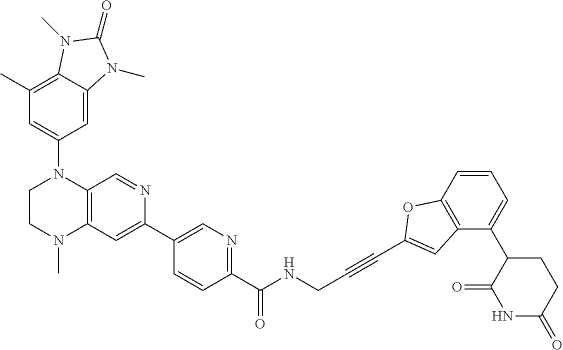 [SMILES: Cc1cc(N2CCN(C)c3cc(-c4ccc(C(=O)NCC#Cc5cc6c(C7CCC(=O)NC7=O)cccc6o5)nc4)ncc32)cc2c1n(C)c(=O)n2C] | N-(3-(4-(2,6-dioxo- piperidin-3- yl)benzofuran-2-yl) prop-2-yn-1-yl)-5-(8- methyl-5-(1,3,7- trimethyl-2-oxo-2,3- dihydro-1H-benzo[d] imidazol-5-yl)- 5,6,7,8-tetrahydro- pteridin-2- yl)picolinamide |
| A220 | 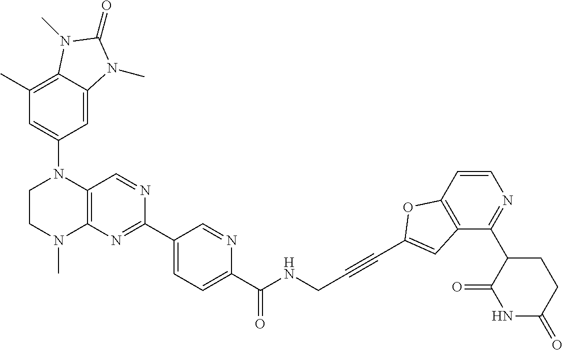 [SMILES: Cc1cc(N2CCN(C)c3nc(-c4ccc(C(=O)NCC#Cc5cc6c(C7CCC(=O)NC7=O)nccc6o5)nc4)ncc32)cc2c1n(C)c(=O)n2C] | N-(3-(4-(2,6-dioxo- piperidin-3-yl)furo[3,2- c]pyridin-2-yl)prop- 2-yn-1-yl)-5-(8- methyl-5-(1,3,7- trimethyl-2-oxo-2,3- dihydro-1H-benzo [d]imidazol-5-yl)- 5,6,7,8-tetrahydro- pteridin-2- yl)picolinamide |
| A221 | 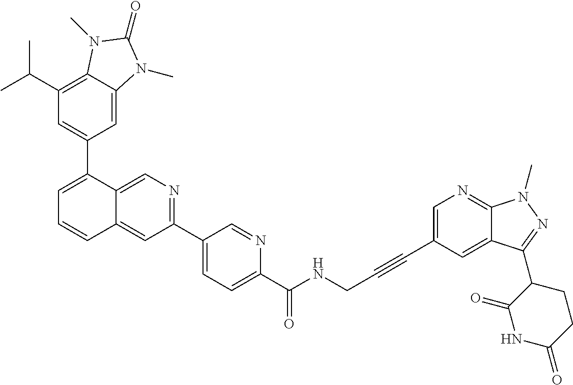 [SMILES: CC(C)c1cc(-c2cccc3cc(-c4ccc(C(=O)NCC#Cc5cnc6c(c5)c(C5CCC(=O)NC5=O)nn6C)nc4)ncc23)cc2c1n(C)c(=O)n2C] | N-(3-(3-(2,6-dioxo- piperidin-3-yl)-1- methyl-1H-pyrazolo [3,4-b]pyridin-5- yl)prop-2-yn-1-yl)- 5-(8-(7-isopropyl-1,3- dimethyl-2-oxo- 2,3-dihydro-1H- benzo[d]imidazol-5- yl)isoquinolin-3- yl)picolinamide |
| A222 | 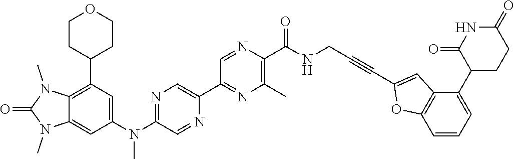 [SMILES: Cc1nc(-c2cnc(N(C)c3cc(C4CCOCC4)c4c(c3)n(C)c(=O)n4C)cn2)cnc1C(=O)NCC#Cc1cc2c(C3CCC(=O)NC3=O)cccc2o1] | 5′-((1,3-dimethyl-2- oxo-7-(tetrahydro-2H- pyran-4-yl)-2,3- dihydro-1H- benzo[d]imidazol-5- yl)(methyl)amino)-N- (3-(4-(2,6-dioxo- piperidin-3-yl) benzofuran-2-yl) prop-2-yn-1-yl)-6- |
| | | methyl-[2,2′-bipyrazine]- |
| | | 5-carboxamide |
| A223 | 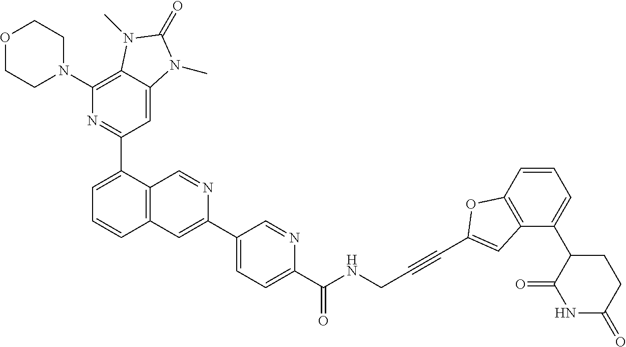 [SMILES: Cn1c(=O)n(C)c2c(N3CCOCC3)nc(-c3cccc4cc(-c5ccc(C(=O)NCC#Cc6cc7c(C8CCC(=O)NC8=O)cccc7o6)nc5)ncc34)cc21] | 5-(8-(1,3-dimethyl-4- morpholino-2-oxo- 2,3-dihydro-1H- imidazo[4,5-c]pyridin-6- yl)isoquinolin-3- yl)-N-(3-(4-(2,6- dioxopiperidin-3- yl)benzofuran-2- yl)prop-2-yn-1- yl)picolinamide |
| A224 | 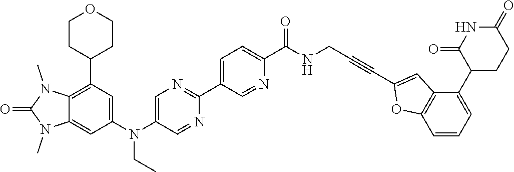 [SMILES: CCN(c1cnc(-c2ccc(C(=O)NCC#Cc3cc4c(C5CCC(=O)NC5=O)cccc4o3)nc2)nc1)c1cc(C2CCOCC2)c2c(c1)n(C)c(=O)n2C] | 5-(5-((1,3-dimethyl- 2-oxo-7-(tetrahydro- 2H-pyran-4-yl)-2,3- dihydro-1H- benzo[d]imidazol-5- yl)(ethyl)amino) pyrimidin-2-yl)-N-(3-(4- (2,6-dioxopiperidin- 3-yl)benzofuran-2- yl)prop-2-yn-1- yl)picolinamide |
| A225 | 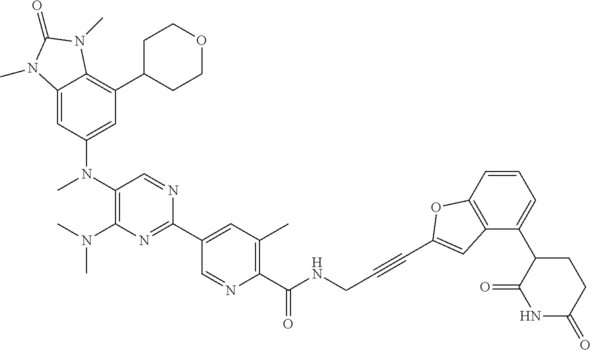 [SMILES: Cc1cc(-c2ncc(N(C)c3cc(C4CCOCC4)c4c(c3)n(C)c(=O)n4C)c(N(C)C)n2)cnc1C(=O)NCC#Cc1cc2c(C3CCC(=O)NC3=O)cccc2o1] | 5-(5-((1,3-dimethyl-2- oxo-7-(tetrahydro- 2H-pyran-4-yl)-2,3- dihydro-1H- benzo[d]imidazol-5- yl)(methyl)amino)-4- (dimethylamino) pyrimidin-2-yl)-N-(3-(4- (2,6-dioxopiperidin- 3-yl)benzofuran-2- yl)prop-2-yn-1-yl)- 3-methylpicolinamide |
| A226 | 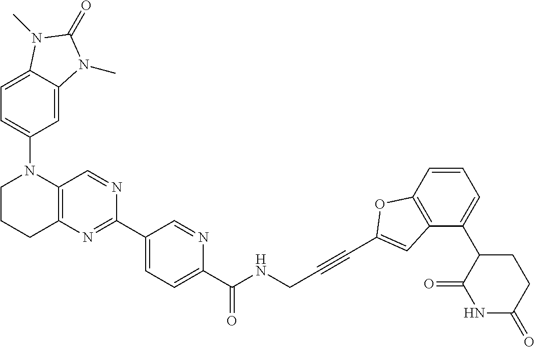 [SMILES: Cn1c(=O)n(C)c2cc(N3CCCc4nc(-c5ccc(C(=O)NCC#Cc6cc7c(C8CCC(=O)NC8=O)cccc7o6)nc5)ncc43)ccc21] | 5-(5-(1,3-dimethyl-2- oxo-2,3-dihydro-1H- benzo[d]imidazol- 5-yl)-5,6,7,8- tetrahydropyrido [3,2-d]pyrimidin-2-yl)- N-(3-(4-(2,6-dioxo- piperidin-3- yl)benzofuran-2- yl)prop-2-yn-1- yl)picolinamide |
| A227 | 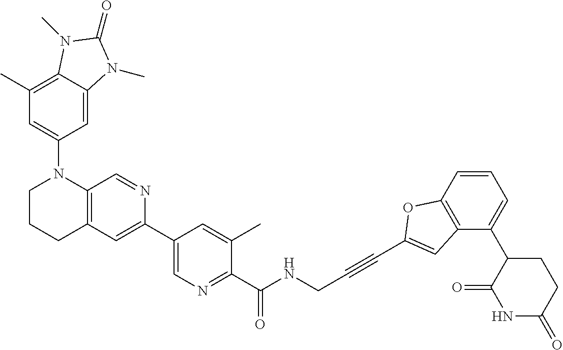 [SMILES: Cc1cc(-c2cc3c(cn2)N(c2cc(C)c4c(c2)n(C)c(=O)n4C)CCC3)cnc1C(=O)NCC#Cc1cc2c(C3CCC(=O)NC3=O)cccc2o1] | N-(3-(4-(2,6-dioxo- piperidin-3- yl)benzofuran-2-yl) prop-2-yn-1-yl)-3- methyl-5-(5-(1,3,7- trimethyl-2-oxo-2,3- dihydro-1H-benzo [d]imidazol-5-yl)- 5,6,7,8-tetrahydro- pyrido[3,2-d]pyrimidin- 2-yl)picolinamide |
| A228 | 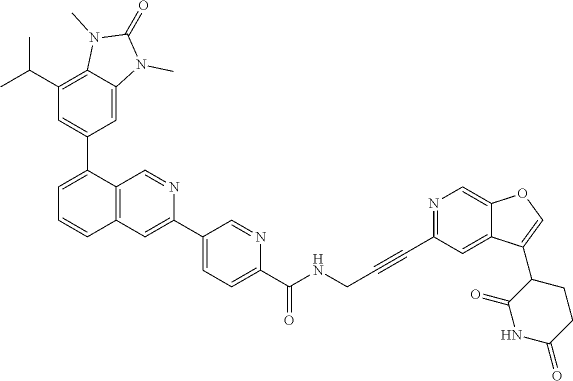 [SMILES: CC(C)c1cc(-c2cccc3cc(-c4ccc(C(=O)NCC#Cc5cc6c(C7CCC(=O)NC7=O)coc6cn5)nc4)ncc23)cc2c1n(C)c(=O)n2C] | N-(3-(3-(2,6-dioxo- piperidin-3-yl)furo[2,3- c]pyridin-5-yl)prop- 2-yn-1-yl)-5-(8-(7- isopropyl-1,3- dimethyl-2-oxo-2,3- dihydro-1H-benzo [d]imidazol-5- yl)isoquinolin-3- yl)picolinamide |
| A229 | 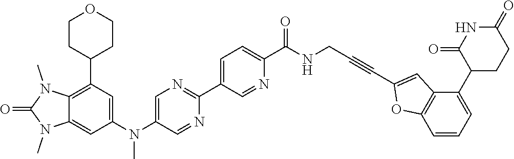 [SMILES: CN(c1cnc(-c2ccc(C(=O)NCC#Cc3cc4c(C5CCC(=O)NC5=O)cccc4o3)nc2)nc1)c1cc(C2CCOCC2)c2c(c1)n(C)c(=O)n2C] | 5-(5-((1,3-dimethyl- 2-oxo-7-(tetrahydro- 2H-pyran-4-yl)- 2,3-dihydro-1H- benzo[d]imidazol-5- yl)(methyl)amino) pyrimidin-2-yl)-N-(3- (4-(2,6-dioxo- piperidin-3-yl) benzofuran-2- |
| | | yl)prop-2-yn-1- |
| | | yl)picolinamide |
| A230 | 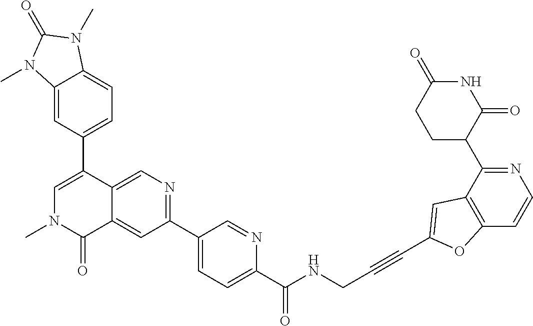 [SMILES: Cn1cc(-c2ccc3c(c2)n(C)c(=O)n3C)c2cnc(-c3ccc(C(=O)NCC#Cc4cc5c(C6CCC(=O)NC6=O)nccc5o4)nc3)cc2c1=O] | 5-(8-(1,3-dimethyl-2- oxo-2,3-dihydro-1H- benzo[d]imidazol-5- yl)-6-methyl-5-oxo- 5,6-dihydro-2,6- naphthyridin-3-yl)-N-(3- (4-(2,6-dioxopiperidin- 3-yl)furo[3,2- c]pyridin-2-yl) prop-2-yn-1- yl)picolinamide |
| A231 | 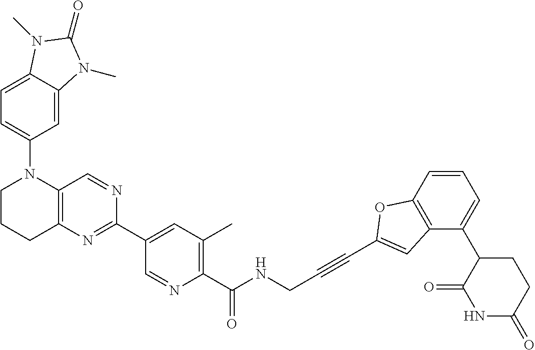 [SMILES: Cc1cc(-c2ncc3c(n2)CCCN3c2ccc3c(c2)n(C)c(=O)n3C)cnc1C(=O)NCC#Cc1cc2c(C3CCC(=O)NC3=O)cccc2o1] | 5-(5-(1,3-dimethyl-2- oxo-2,3-dihydro-1H- benzo[d]imidazol-5- yl)-5,6,7,8-tetra- hydropyrido[3,2-d] pyrimidin-2-yl)- N-(3-(4-(2,6-dioxo- piperidin-3- yl)benzofuran-2-yl) prop-2-yn-1-yl)-3- methylpicolinamide |
| A232 | 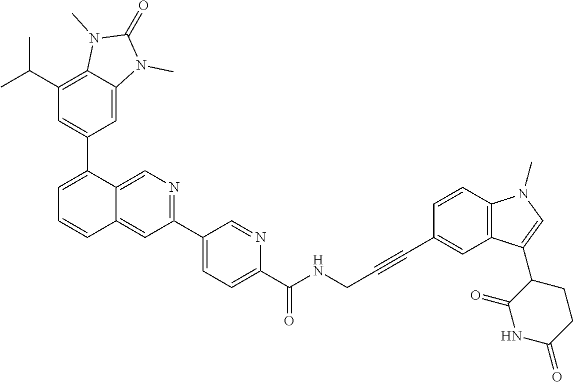 [SMILES: CC(C)c1cc(-c2cccc3cc(-c4ccc(C(=O)NCC#Cc5ccc6c(c5)c(C5CCC(=O)NC5=O)cn6C)nc4)ncc23)cc2c1n(C)c(=O)n2C] | N-(3-(3-(2,6-dioxo- piperidin-3-yl)-1- methyl-1H-pyrrolo [2,3-c]pyridin-5- yl)prop-2-yn-1-yl)- 5-(8-(7-isopropyl-1,3- dimethyl-2-oxo-2,3- dihydro-1H- benzo[d]imidazol- 5-yl)isoquinolin-3- yl)picolinamide |
| A233 | 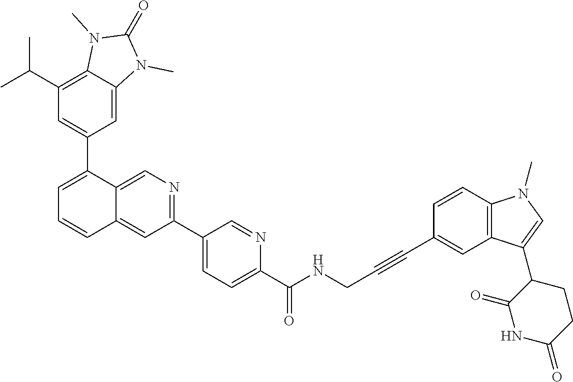 [SMILES: CC(C)c1cc(-c2cccc3cc(-c4ccc(C(=O)NCC#Cc5ccc6c(c5)c(C5CCC(=O)NC5=O)cn6C)nc4)ncc23)cc2c1n(C)c(=O)n2C] | N-(3-(7-cyano-3- (2,6-dioxopiperidin-3- yl)benzofuran-5-yl) prop-2-yn-1-yl)-5-(8- (7-isopropyl-1,3- dimethyl-2-oxo-2,3- dihydro-1H-benzo [d]imidazol-5- yl)isoquinolin-3- yl)picolinamide |
| A234 | 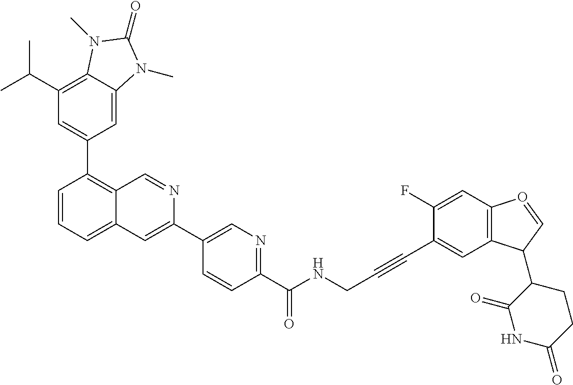 | N-(3-(3-(2,6-dioxo- piperidin-3-yl)-6- fluorobenzofuran-5- yl)prop-2-yn-1-yl)-5- (8-(7-isopropyl-1,3- dimethyl-2-oxo-2,3- dihydro-1H-benzo [d]imidazol-5- yl)isoquinolin-3- yl)picolinamide |
| A235 | 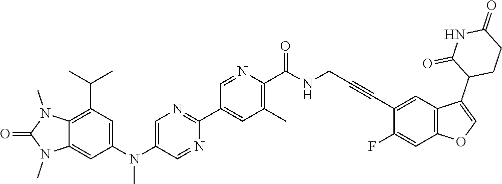 [SMILES: Cc1cc(-c2ncc(N(C)c3cc(C(C)C)c4c(c3)n(C)c(=O)n4C)cn2)cnc1C(=O)NCC#Cc1cc2c(C3CCC(=O)NC3=O)coc2cc1F] | N-(3-(3-(2,6-dioxo- piperidin-3-yl)-6- fluorobenzofuran-5- yl)prop-2-yn-1-yl)-5- (5-((7-isopropyl-1,3- dimethyl-2-oxo-2,3- dihydro-1H-benzo [d]imidazol-5- yl)(methyl)amino) pyrimidin-2-yl)-3- methylpicolinamide |
| A236 | 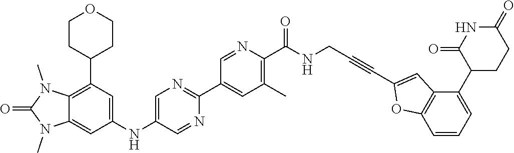 [SMILES: Cc1cc(-c2ncc(Nc3cc(C4CCOCC4)c4c(c3)n(C)c(=O)n4C)cn2)cnc1C(=O)NCC#Cc1cc2c(C3CCC(=O)NC3=O)cccc2o1] | 5-(5-((1,3-dimethyl- 2-oxo-7-(tetrahydro- 2H-pyran-4-yl)-2,3- dihydro-1H- benzo[d]imidazol-5- yl)amino)pyrimidin- 2-yl)-N-(3-(4-(2,6- dioxopiperidin-3- yl)benzofuran-2-yl) prop-2-yn-1-yl)-3- |
| | | methylpicolinamide |
| A237 | 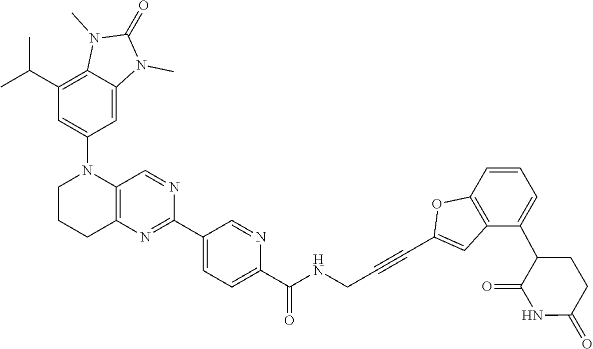 [SMILES: CC(C)c1cc(N2CCCc3nc(-c4ccc(C(=O)NCC#Cc5cc6c(C7CCC(=O)NC7=O)cccc6o5)nc4)ncc32)cc2c1n(C)c(=O)n2C] | N-(3-(4-(2,6-dioxo- piperidin-3- yl)benzofuran-2-yl) prop-2-yn-1-yl)-5-(5- (7-isopropyl-1,3- dimethyl-2-oxo-2,3- dihydro-1H-benzo [d]imidazol-5-yl)- 5,6,7,8-tetrahydro- pyrido[3,2-d]pyrimidin- 2-yl)picolinamide |
| A238 | 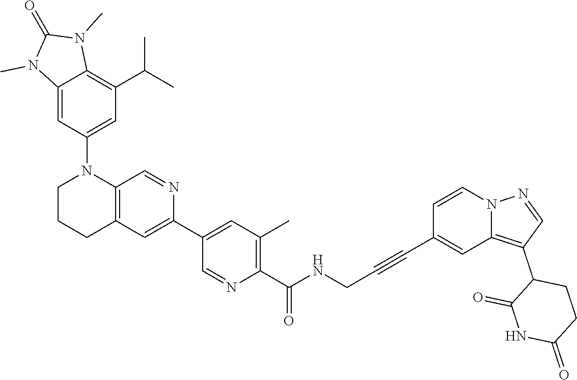 [SMILES: Cc1cc(-c2cc3c(cn2)N(c2cc(C(C)C)c4c(c2)n(C)c(=O)n4C)CCC3)cnc1C(=O)NCC#Cc1ccn2ncc(C3CCC(=O)NC3=O)c2c1] | N-(3-(3-(2,6-dioxo- piperidin-3- yl)pyrazolo[1,5-a] pyridin-5-yl)prop-2-yn- 1-yl)-5-(1-(7-isopropyl- 1,3-dimethyl-2- oxo-2,3-dihydro-1H- benzo[d]imidazol-5- yl)-1,2,3,4-tetrahydro- 1,7-naphthyridin-6-yl)- 3-methylpicolinamide |
| A239 | 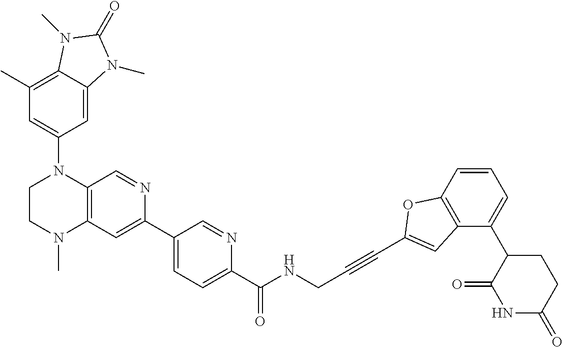 [SMILES: Cc1cc(N2CCN(C)c3cc(-c4ccc(C(=O)NCC#Cc5cc6c(C7CCC(=O)NC7=O)cccc6o5)nc4)ncc32)cc2c1n(C)c(=O)n2C] | N-(3-(4-(2,6-dioxo- piperidin-3- yl)benzofuran-2-yl) prop-2-yn-1-yl)-5-(1- methyl-4-(1,3,7- trimethyl-2-oxo-2,3- dihydro-1H-benzo [d]imidazol-5-yl)- 1,2,3,4-tetrahydro- pyrido[3,4-b]pyrazin-7- yl)picolinamide |
| A240 | 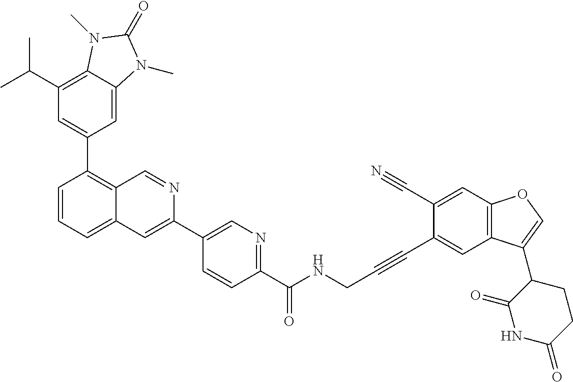 [SMILES: CC(C)c1cc(-c2cccc3cc(-c4ccc(C(=O)NCC#Cc5cc6c(C7CCC(=O)NC7=O)coc6cc5C#N)nc4)ncc23)cc2c1n(C)c(=O)n2C] | N-(3-(6-cyano-3- (2,6-dioxopiperidin-3- yl)benzofuran-5-yl) prop-2-yn-1-yl)-5-(8- (7-isopropyl-1,3- dimethyl-2-oxo-2,3- dihydro-1H-benzo [d]imidazol-5- yl)isoquinolin-3- yl)picolinamide |
| A241 | 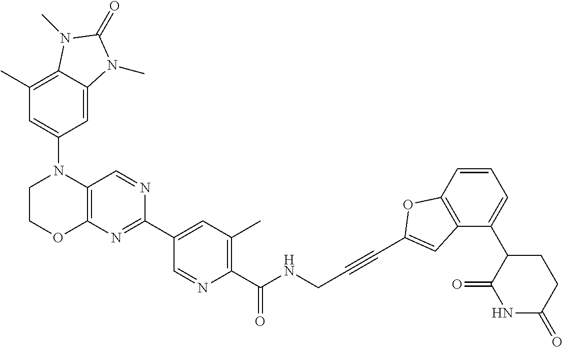 [SMILES: Cc1cc(-c2ncc3c(n2)OCCN3c2cc(C)c3c(c2)n(C)c(=O)n3C)cnc1C(=O)NCC#Cc1cc2c(C3CCC(=O)NC3=O)cccc2o1] | N-(3-(4-(2,6-dioxo- piperidin-3- yl)benzofuran-2-yl) prop-2-yn-1-yl)-3- methyl-5-(5-(1,3,7- trimethyl-2-oxo-2,3- dihydro-1H-benzo [d]imidazol-5-yl)-6,7- dihydro-5H-pyrimido [4,5-b][1,4]oxazin- 2-yl)picolinamide |
| A242 | 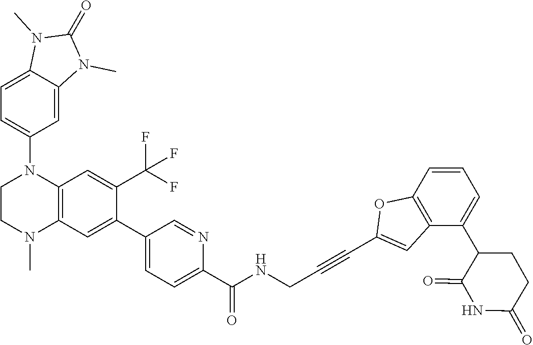 [SMILES: CN1CCN(c2ccc3c(c2)n(C)c(=O)n3C)c2cc(C(F)(F)F)c(-c3ccc(C(=O)NCC#Cc4cc5c(C6CCC(=O)NC6=O)cccc5o4)nc3)cc21] | 5-(1-(1,3-dimethyl-2- oxo-2,3-dihydro-1H- benzo[d]imidazol- 5-yl)-4-methyl-7- (trifluoromethyl)-1,2,3,4- tetrahydroquinoxalin- 6-yl)-N-(3-(4-(2,6- dioxopiperidin-3- yl)benzofuran-2- yl)prop-2-yn-1- yl)picolinamide |
| A243 | 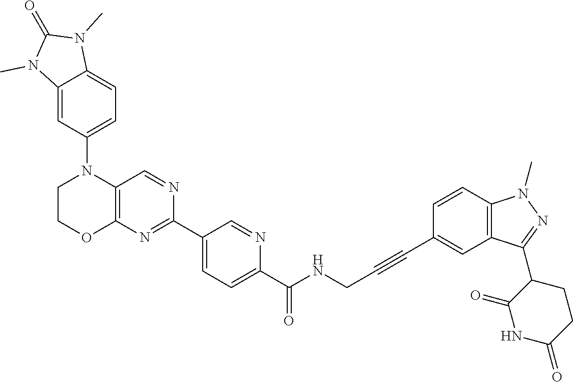 [SMILES: Cn1nc(C2CCC(=O)NC2=O)c2cc(C#CCNC(=O)c3ccc(-c4ncc5c(n4)OCCN5c4ccc5c(c4)n(C)c(=O)n5C)cn3)ccc21] | 5-(5-(1,3-dimethyl-2- oxo-2,3-dihydro-1H- benzo[d]imidazol-5- yl)-6,7-dihydro-5H- pyrimido[4,5-b][1,4] oxazin-2-yl)-N-(3-(3- (2,6-dioxopiperidin- 3-yl)-1-methyl-1H- indazol-5-yl)prop- 2-yn-1-yl)picolinamide |
| A244 | 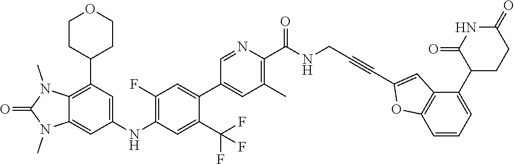 [SMILES: Cc1cc(-c2cc(F)c(Nc3cc(C4CCOCC4)c4c(c3)n(C)c(=O)n4C)cc2C(F)(F)F)cnc1C(=O)NCC#Cc1cc2c(C3CCC(=O)NC3=O)cccc2o1] | 5-(4-((1,3-dimethyl- 2-oxo-7-(tetrahydro- 2H-pyran-4-yl)-2,3- dihydro-1H- benzo[d]imidazol-5- yl)amino)-5-fluoro-2- (trifluoromethyl) phenyl)-N-(3-(4-(2,6- dioxopiperidin-3-yl) benzofuran-2- yl)prop-2-yn-1-yl)- |
| | | 3-methylpicolinamide |
| A245 | 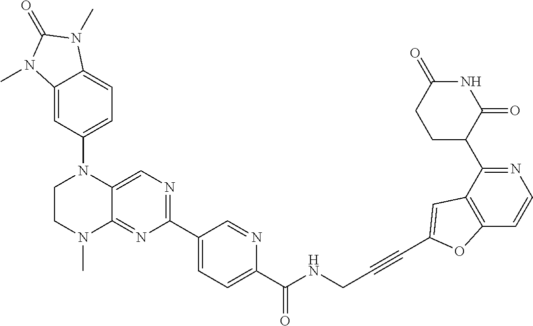 [SMILES: CN1CCN(c2ccc3c(c2)n(C)c(=O)n3C)c2cnc(-c3ccc(C(=O)NCC#Cc4cc5c(C6CCC(=O)NC6=O)nccc5o4)nc3)nc21] | 5-(5-(1,3-dimethyl-2- oxo-2,3-dihydro-1H- benzo[d]imidazol-5- yl)-8-methyl-5,6,7,8- tetrahydropteridin-2- yl)-N-(3-(4-(2,6- dioxopiperidin-3-yl) furo[3,2-c]pyridin-2- yl)prop-2-yn-1-yl) picolinamide |
| A246 | 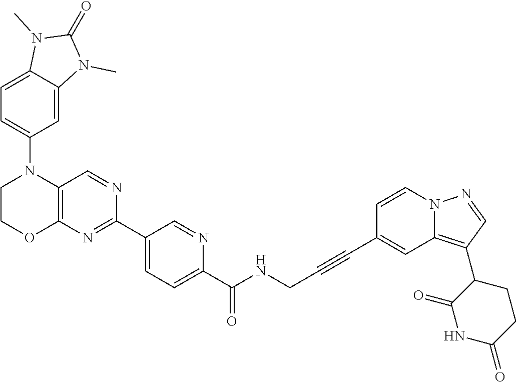 [SMILES: Cn1c(=O)n(C)c2cc(N3CCOc4nc(-c5ccc(C(=O)NCC#Cc6ccn7ncc(C8CCC(=O)NC8=O)c7c6)nc5)ncc43)ccc21] | 5-(5-(1,3-dimethyl-2- oxo-2,3-dihydro-1H- benzo[d]imidazol-5- yl)-6,7-dihydro-5H- pyrimido[4,5-b][1,4] oxazin-2-yl)-N-(3-(3- (2,6-dioxopiperidin- 3-yl)pyrazolo[1,5- a]pyridin-5-yl) prop-2-yn-1- yl)picolinamide |
| A247 | 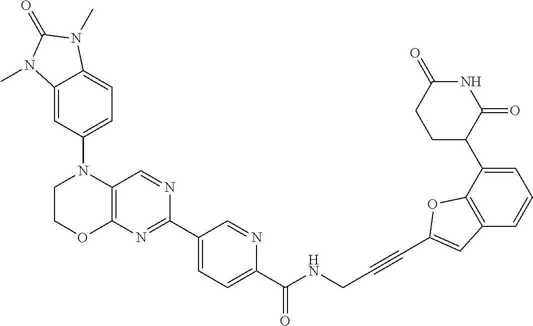 [SMILES: Cn1c(=O)n(C)c2cc(N3CCOc4nc(-c5ccc(C(=O)NCC#Cc6cc7cccc(C8CCC(=O)NC8=O)c7o6)nc5)ncc43)ccc21] | 5-(5-(1,3-dimethyl-2- oxo-2,3-dihydro-1H- benzo[d]imidazol-5- yl)-6,7-dihydro-5H- pyrimido[4,5-b][1,4] oxazin-2-yl)-N-(3-(7- (2,6-dioxopiperidin- 3-yl)benzofuran-2- yl)prop-2-yn-1-yl) picolinamide |
| A248 | 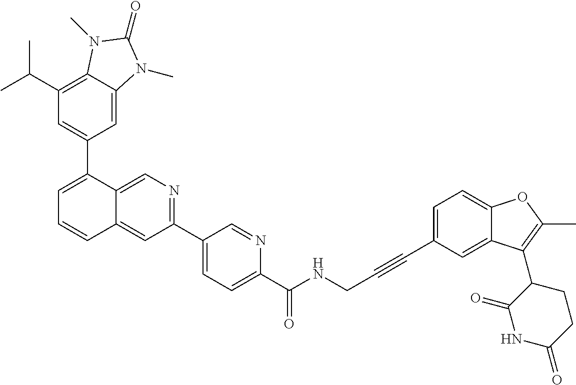 [SMILES: Cc1oc2ccc(C#CCNC(=O)c3ccc(-c4cc5cccc(-c6cc(C(C)C)c7c(c6)n(C)c(=O)n7C)c5cn4)cn3)cc2c1C1CCC(=O)NC1=O] | N-(3-(3-(2,6-dioxo- piperidin-3-yl)-2- methylbenzofuran-5- yl)prop-2-yn-1-yl)-5- (8-(7-isopropyl-1,3- dimethyl-2-oxo-2,3- dihydro-1H-benzo [d]imidazol-5- yl)isoquinolin-3-yl) picolinamide |
| A249 | 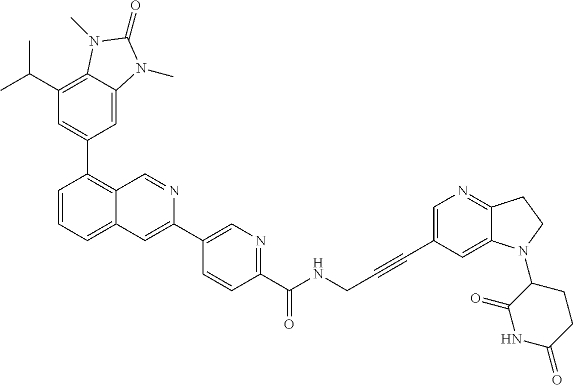 [SMILES: CC(C)c1cc(-c2cccc3cc(-c4ccc(C(=O)NCC#Cc5cnc6c(c5)N(C5CCC(=O)NC5=O)CC6)nc4)ncc23)cc2c1n(C)c(=O)n2C] | N-(3-(1-(2,6-dioxo- piperidin-3-yl)-2,3- dihydro-1H-pyrrolo [3,2-b]pyridin-6- yl)prop-2-yn-1-yl)- 5-(8-(7-isopropyl-1,3- dimethyl-2-oxo-2,3- dihydro-1H- benzo[d]imidazol-5- yl)isoquinolin-3- yl)picolinamide |
| A251 | 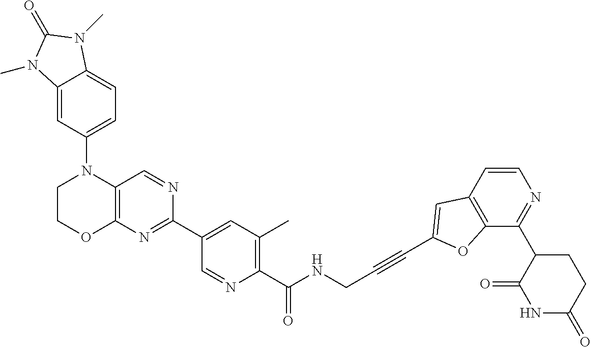 [SMILES: Cc1cc(-c2ncc3c(n2)OCCN3c2ccc3c(c2)n(C)c(=O)n3C)cnc1C(=O)NCC#Cc1cc2ccnc(C3CCC(=O)NC3=O)c2o1] | 5-(5-(1,3-dimethyl-2- oxo-2,3-dihydro-1H- benzo[d]imidazol-5- yl)-6,7-dihydro-5H- pyrimido[4,5-b][1,4] oxazin-2-yl)-N-(3-(7- (2,6-dioxopiperidin- 3-yl)furo[2,3- c]pyridin-2-yl)prop- 2-yn-1-yl)-3- methylpicolinamide |
| A252 | 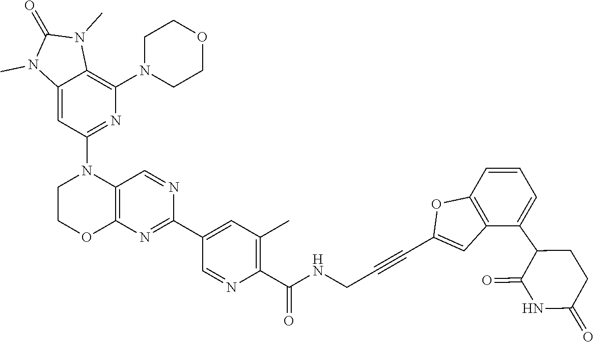 [SMILES: Cc1cc(-c2ncc3c(n2)OCCN3c2cc3c(c(N4CCOCC4)n2)n(C)c(=O)n3C)cnc1C(=O)NCC#Cc1cc2c(C3CCC(=O)NC3=O)cccc2o1] | 5-(5-(1,3-dimethyl-2- oxo-7-(tetrahydro- 2H-pyran-4-yl)-2,3- dihydro-1H- benzo[d]imidazol-5- yl)-6,7-dihydro-5H- pyrimido[4,5-b][1,4] oxazin-2-yl)-N-(3-(4- (2,6-dioxopiperidin- 3-yl)benzofuran-2- yl)prop-2-yn-1-yl)- 3-methylpicolinamide |
| A253 | 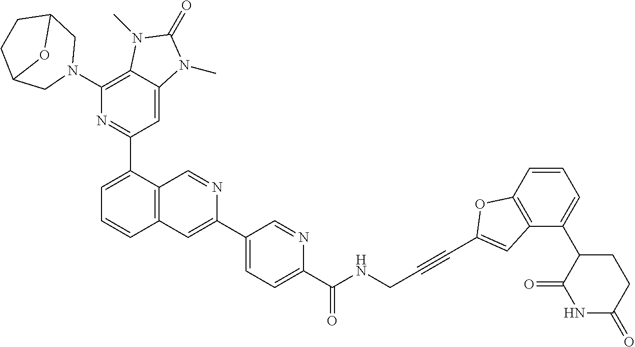 [SMILES: Cn1c(=O)n(C)c2c(N3CC4CCC(C3)O4)nc(-c3cccc4cc(-c5ccc(C(=O)NCC#Cc6cc7c(C8CCC(=O)NC8=O)cccc7o6)nc5)ncc34)cc21] | 5-(8-(4-(8-oxa-3- azabicyclo[3.2.1]octan- 3-yl)-1,3-dimethyl-2- oxo-2,3-dihydro-1H- imidazo[4,5-c]pyridin- 6-yl)isoquinolin-3- yl)-N-(3-(4-(2,6- dioxopiperidin-3- yl)benzofuran-2- yl)prop-2-yn-1- yl)picolinamide |
| A254 | 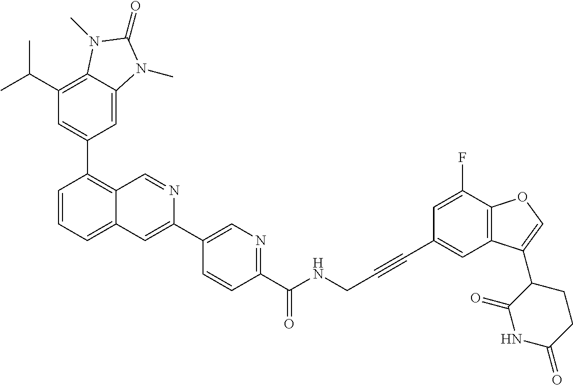 [SMILES: CC(C)c1cc(-c2cccc3cc(-c4ccc(C(=O)NCC#Cc5cc(F)c6occ(C7CCC(=O)NC7=O)c6c5)nc4)ncc23)cc2c1n(C)c(=O)n2C] | N-(3-(3-(2,6-dioxo- piperidin-3-yl)-7- fluorobenzofuran-5- yl)prop-2-yn-1-yl)-5- (8-(7-isopropyl-1,3- dimethyl-2-oxo-2,3- dihydro-1H-benzo [d]imidazol-5- yl)isoquinolin-3- yl)picolinamide |
| A255 | 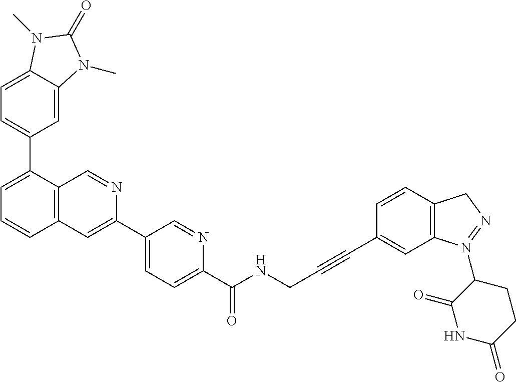 | 5-(8-(1,3-dimethyl-2- oxo-2,3-dihydro-1H- benzo[d]imidazol-5- yl)isoquinolin-3-yl)- N-(3-(1-(2,6-dioxo- piperidin-3-yl)-1H- indazol-6-yl)prop- 2-yn-1-yl)picolinamide |
| A256 | 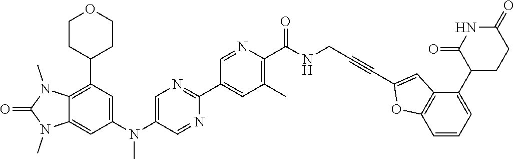 [SMILES: Cc1cc(-c2ncc(N(C)c3cc(C4CCOCC4)c4c(c3)n(C)c(=O)n4C)cn2)cnc1C(=O)NCC#Cc1cc2c(C3CCC(=O)NC3=O)cccc2o1] | 5-(5-((1,3-dimethyl- 2-oxo-7-(tetrahydro- 2H-pyran-4-yl)- 2,3-dihydro-1H- benzo[d]imidazol-5- yl)(methyl)amino) pyrimidin-2-yl)-N-(3- (4-(2,6-dioxo- piperidin-3-yl)benzo- furan-2- |
| | | yl)prop-2-yn-1-yl)-3- |
| | | methylpicolinamide |
| A257 | 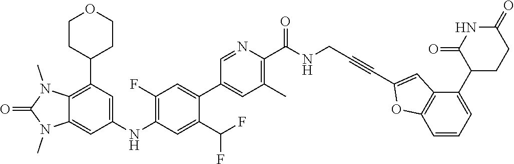 [SMILES: Cc1cc(-c2cc(F)c(Nc3cc(C4CCOCC4)c4c(c3)n(C)c(=O)n4C)cc2C(F)F)cnc1C(=O)NCC#Cc1cc2c(C3CCC(=O)NC3=O)cccc2o1] | 5-(2-(difluoromethyl)- 4-((1,3-dimethyl-2- oxo-7-(tetrahydro-2H- pyran-4-yl)-2,3- dihydro-1H-benzo [d]imidazol-5- yl)amino)-5-fluoro- phenyl)-N-(3-(4-(2,6- dioxopiperidin-3-yl) benzofuran-2- |
| | | yl)prop-2-yn-1-yl)- |
| | | 3-methylpicolinamide |
| A258 | 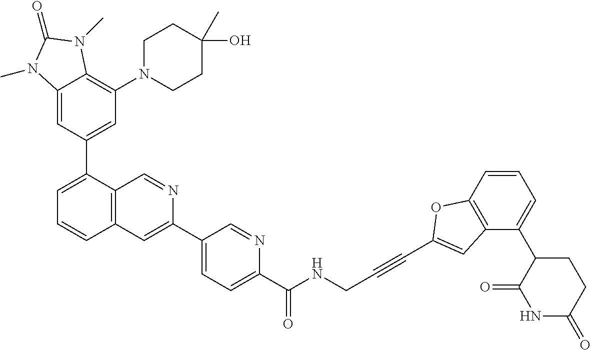 [SMILES: Cn1c(=O)n(C)c2c(N3CCC(C)(O)CC3)cc(-c3cccc4cc(-c5ccc(C(=O)NCC#Cc6cc7c(C8CCC(=O)NC8=O)cccc7o6)nc5)ncc34)cc21] | N-(3-(4-(2,6-dioxo- piperidin-3- yl)benzofuran-2-yl) prop-2-yn-1-yl)-5-(8- (4-(4-hydroxy-4- methylpiperidin-1-yl)- 1,3-dimethyl-2-oxo- 2,3-dihydro-1H- imidazo[4,5-c]pyridin- 6-yl)isoquinolin-3- yl)picolinamide |
| A259 | 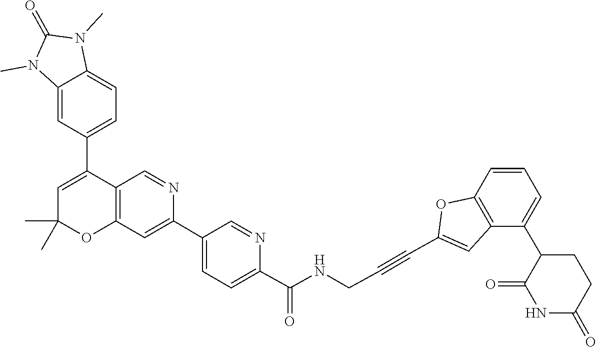 [SMILES: Cn1c(=O)n(C)c2cc(C3=CC(C)(C)Oc4cc(-c5ccc(C(=O)NCC#Cc6cc7c(C8CCC(=O)NC8=O)cccc7o6)nc5)ncc43)ccc21] | 5-(4-(1,3-dimethyl-2- oxo-2,3-dihydro-1H- benzo[d]imidazol-5- yl)-2,2-dimethyl-2H- pyrano[3,2-c]pyridin- 7-yl)-N-(3-(4-(2,6- dioxopiperidin-3- yl)benzofuran-2- yl)prop-2-yn-1- yl)picolinamide |
| A260 | 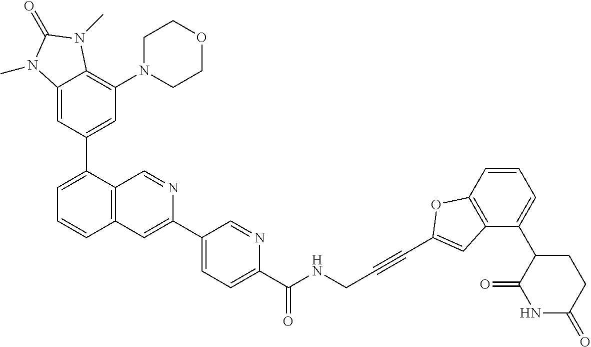 [SMILES: Cn1c(=O)n(C)c2c(N3CCOCC3)cc(-c3cccc4cc(-c5ccc(C(=O)NCC#Cc6cc7c(C8CCC(=O)NC8=O)cccc7o6)nc5)ncc34)cc21] | N-(3-(4-(2,6-dioxo- piperidin-3-yl) benzofuran-2-yl)prop- 2-yn-1-yl)-5-(8-(1- ethyl-3-methyl- 4-morpholino-2- oxo-2,3-dihydro-1H- imidazo[4,5-c]pyridin- 6-yl)isoquinolin-3- yl)picolinamide |
| A261 | 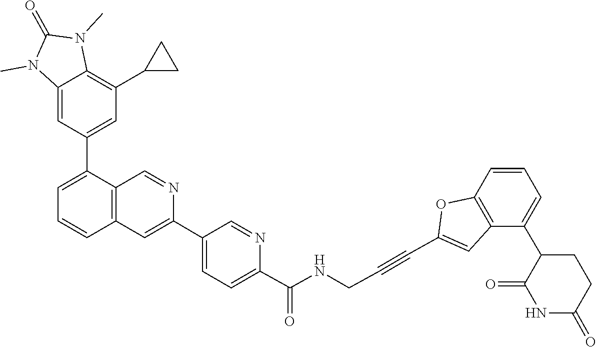 [SMILES: Cn1c(=O)n(C)c2c(C3CC3)cc(-c3cccc4cc(-c5ccc(C(=O)NCC#Cc6cc7c(C8CCC(=O)NC8=O)cccc7o6)nc5)ncc34)cc21] | 5-(8-(7-cyclopropyl-1,3- dimethyl-2-oxo- 2,3-dihydro- 1H-benzo[d]imidazol-5- yl)isoquinolin-3- yl)-N-(3-(4-(2,6- dioxopiperidin-3-yl) benzofuran-2-yl)prop-2- yn-1-yl)picolinamide |
| A262 | 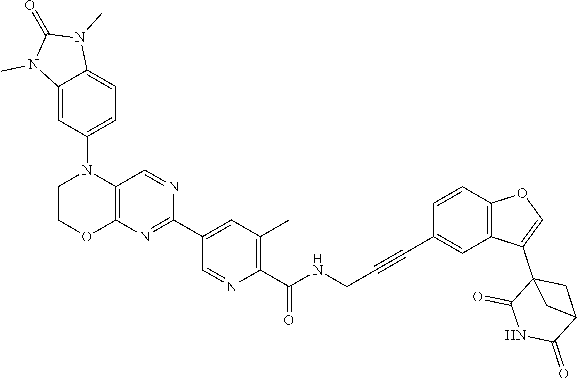 [SMILES: Cc1cc(-c2ncc3c(n2)OCCN3c2ccc3c(c2)n(C)c(=O)n3C)cnc1C(=O)NCC#Cc1ccc2occ(C34CC(C3)C(=O)NC4=O)c2c1] | 5-(5-(1,3-dimethyl-2-oxo- 2,3-dihydro-1H-benzo[d] imidazol-5-yl)- 6,7-dihydro-5H- pyrimido[4,5-b] [1,4]oxazin- 2-yl)-N-(3-(3- (2,4-dioxo- 3-azabicyclo[3.1.1] heptan-1- yl)benzofuran- 5-yl)prop-2-yn- 1-yl)-3- methylpicolinamide |
| A263 | 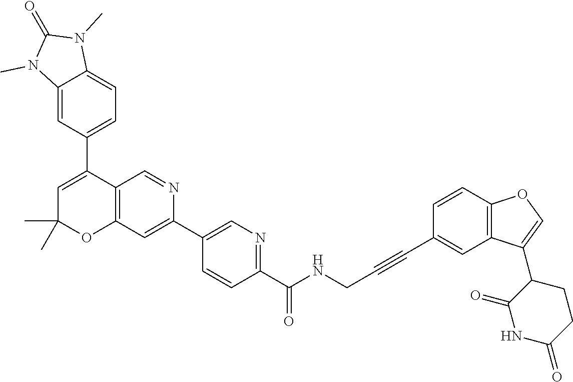 [SMILES: Cn1c(=O)n(C)c2cc(C3=CC(C)(C)Oc4cc(-c5ccc(C(=O)NCC#Cc6ccc7occ(C8CCC(=O)NC8=O)c7c6)nc5)ncc43)ccc21] | 5-(4-(1,3-dimethyl-2-oxo- 2,3-dihydro-1H-benzo[d] imidazol-5-yl)- 2,2-dimethyl-2H- pyrano[3,2-c]pyridin-7- yl)-N-(3-(3-(2,6- dioxopiperidin-3-yl) benzofuran-5-yl)prop-2- yn-1-yl)picolinamide |
| A264 | 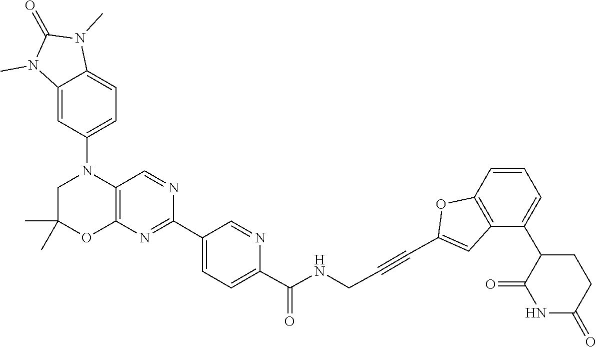 [SMILES: Cn1c(=O)n(C)c2cc(N3CC(C)(C)Oc4nc(-c5ccc(C(=O)NCC#Cc6cc7c(C8CCC(=O)NC8=O)cccc7o6)nc5)ncc43)ccc21] | 5-(5-(1,3-dimethyl- 2-oxo-2,3-dihydro- 1H-benzo[d] imidazol-5-yl)-7,7- dimethyl-6,7- dihydro-5H-pyrimido [4,5-b][1,4]oxazin- 2-yl)-N-(3-(4-(2,6- dioxopiperidin-3- yl)benzofuran- 2-yl)prop-2-yn-1- yl)picolinamide |
| A265 | 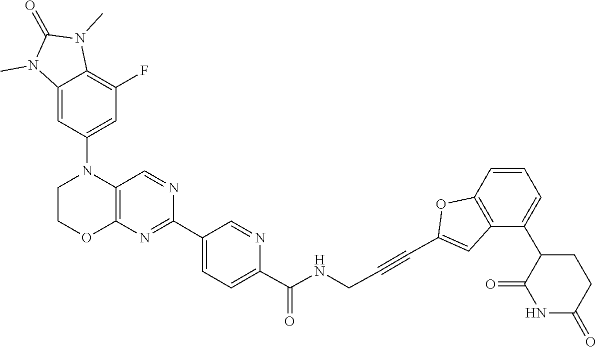 [SMILES: Cn1c(=O)n(C)c2c(F)cc(N3CCOc4nc(-c5ccc(C(=O)NCC#Cc6cc7c(C8CCC(=O)NC8=O)cccc7o6)nc5)ncc43)cc21] | N-(3-(4-(2,6-dioxo- piperidin-3-yl) benzofuran-2-yl)prop- 2-yn-1-yl)-5-(5- (7-fluoro-1,3- dimethyl-2-oxo- 2,3-dihydro-1H- benzo[d]imidazol- 5-yl)-6,7-dihydro- 5H-pyrimido[4,5- b][1,4]oxazin- 2-yl)picolinamide |
| A266 | 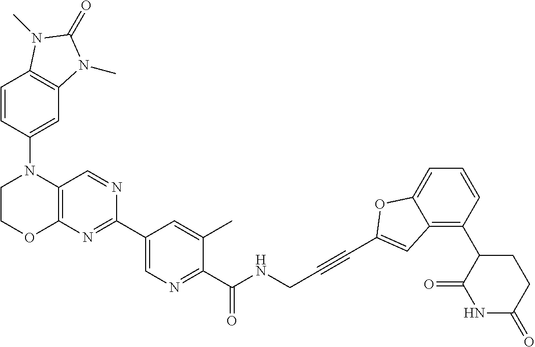 [SMILES: Cc1cc(-c2ncc3c(n2)OCCN3c2ccc3c(c2)n(C)c(=O)n3C)cnc1C(=O)NCC#Cc1cc2c(C3CCC(=O)NC3=O)cccc2o1] | 5-(5-(1,3-dimethyl- 2-oxo-2,3-dihydro- 1H-imidazo[4,5-b] pyridin-6-yl)-6,7- dihydro-5H- pyrimido[4,5-b] [1,4]oxazin-2- yl)-N-(3-(4-(2,6- dioxopiperidin-3-yl) benzofuran-2- yl)prop-2-yn-1-yl)-3- methylpicolinamide |
| A267 | 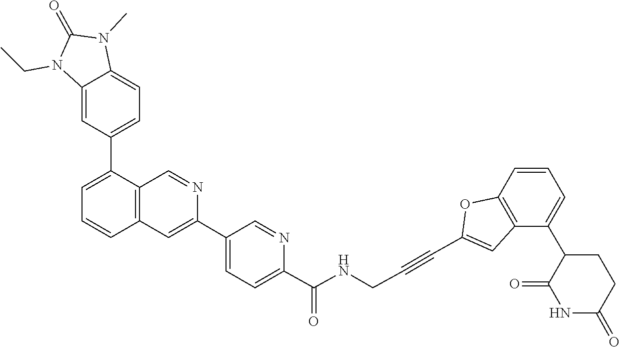 [SMILES: CCn1c(=O)n(C)c2ccc(-c3cccc4cc(-c5ccc(C(=O)NCC#Cc6cc7c(C8CCC(=O)NC8=O)cccc7o6)nc5)ncc34)cc21] | N-(3-(4-(2,6-dioxo- piperidin-3-yl) benzofuran-2-yl) prop-2-yn-1-yl)- 5-(8-(3-ethyl-1- methyl-2-oxo-2,3- dihydro-1H-benzo [d]imidazol-5- yl)isoquinolin-3- yl)picolinamide |
| A268 | 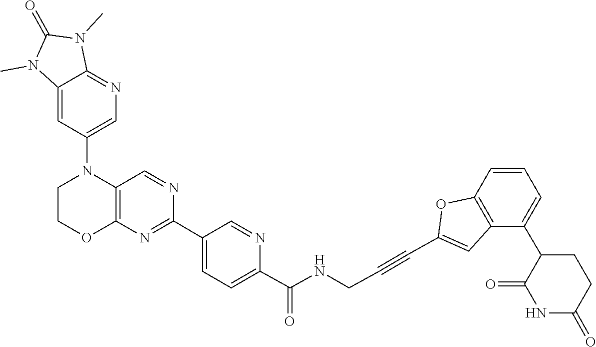 [SMILES: Cn1c(=O)n(C)c2ncc(N3CCOc4nc(-c5ccc(C(=O)NCC#Cc6cc7c(C8CCC(=O)NC8=O)cccc7o6)nc5)ncc43)cc21] | 5-(5-(1,3-dimethyl- 2-oxo-2,3-dihydro- 1H-imidazo[4,5- b]pyridin-6-yl)- 6,7-dihydro-5H- pyrimido[4,5-b] [1,4]oxazin- 2-yl)-N-(3-(4-(2,6- dioxopiperidin-3- yl)benzofuran-2- yl)prop-2-yn-1- yl)picolinamide |
| A269 | 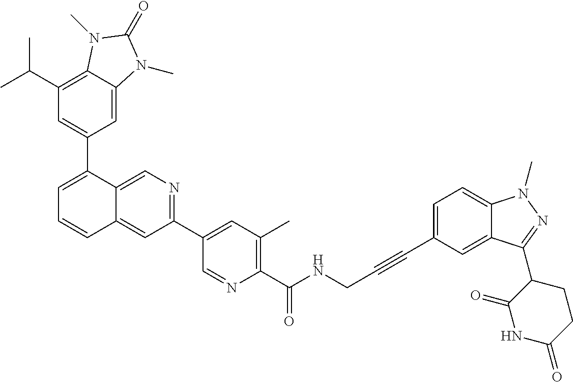 [SMILES: Cc1cc(-c2cc3cccc(-c4cc(C(C)C)c5c(c4)n(C)c(=O)n5C)c3cn2)cnc1C(=O)NCC#Cc1ccc2c(c1)c(C1CCC(=O)NC1=O)nn2C] | N-(3-(3-(2,6- dioxopiperidin- 3-yl)-1-methyl- 1H-indazol- 5-yl)prop-2-yn- 1-yl)-5-(8-(7- isopropyl-1,3- dimethyl-2- oxo-2,3-dihydro- 1H-benzo[d] imidazol-5-yl)iso- quinolin-3-yl)- 3-methylpicolinamide |
| A270 | 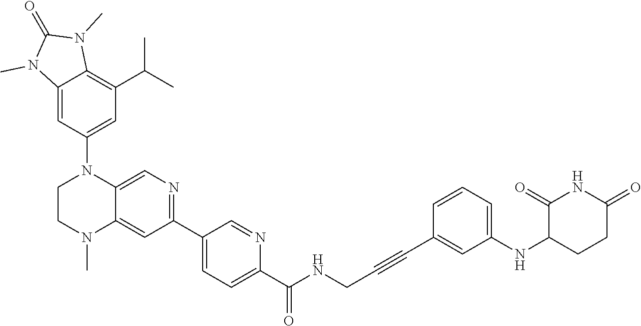 [SMILES: CC(C)c1cc(N2CCN(C)c3cc(-c4ccc(C(=O)NCC#Cc5cccc(NC6CCC(=O)NC6=O)c5)nc4)ncc32)cc2c1n(C)c(=O)n2C] | N-(3-(3-((2,6- dioxopiperidin-3- yl)amino)phenyl) prop-2-yn-1-yl)-5- (4-(7-isopropyl- 1,3-dimethyl-2- oxo-2,3-dihydro- 1H-benzo[d] imidazol-5-yl)- 1-methyl-1,2,3,4- tetrahydro- pyrido[3,4-b]pyrazin- 7-yl)picolinamide |
| A271 | 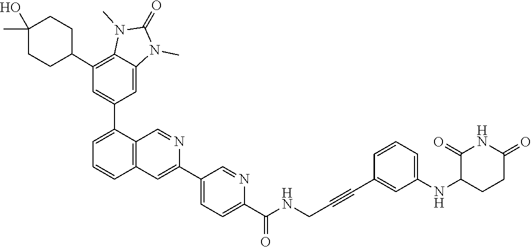 [SMILES: Cn1c(=O)n(C)c2c(C3CCC(C)(O)CC3)cc(-c3cccc4cc(-c5ccc(C(=O)NCC#Cc6cccc(NC7CCC(=O)NC7=O)c6)nc5)ncc34)cc21] | N-(3-(3-((2,6- dioxopiperidin-3- yl)amino)phenyl) prop-2-yn-1-yl)-5- (8-(7-(4-hydroxy- 4-methylpiperidin- 1-yl)-1,3- dimethyl-2-oxo- 2,3-dihydro-1H- benzo[d]imidazol- 5-yl)isoquinolin-3- yl)picolinamide |

TABLE 2

| Com- | | |
|---|---|---|
| pound | | |
| No. | Chemical Structure | Chemical Name |
| B1 | 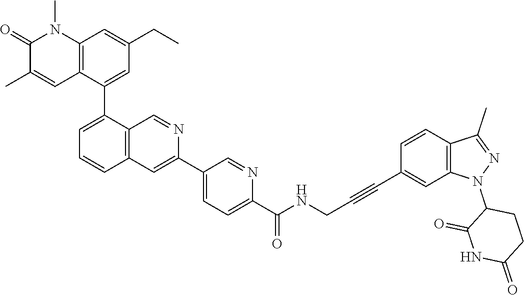 [SMILES: CCc1cc(-c2cccc3cc(-c4ccc(C(=O)NCC#Cc5ccc6c(C)nn(C7CCC(=O)NC7=O)c6c5)nc4)ncc23)c2cc(C)c(=O)n(C)c2c1] | N-(3-(1-(2,6-dioxopiperidin-3-yl)-3-methyl-1H- indazol-6-yl)prop-2-yn-1-yl)-5-(8-(7-ethyl-1,3- dimethyl-2-oxo-1,2-dihydroquinolin-5-yl) isoquinolin-3-yl)picolinamide |
| B2 | 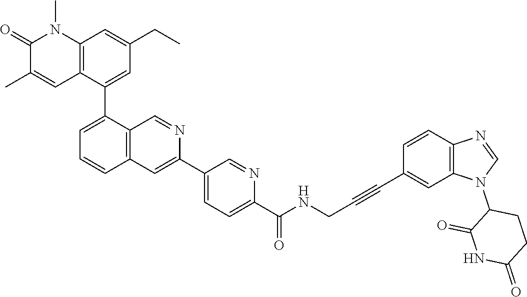 [SMILES: CCc1cc(-c2cccc3cc(-c4ccc(C(=O)NCC#Cc5ccc6ncn(C7CCC(=O)NC7=O)c6c5)nc4)ncc23)c2cc(C)c(=O)n(C)c2c1] | N-(3-(1-(2,6-dioxopiperidin-3-yl)-1H- benzo[d]imidazol-6-yl)prop-2-yn-1-yl)-5-(8-(7- ethyl-1,3-dimethyl-2-oxo-1,2-dihydroquinolin- 5-yl)isoquinolin-3-yl)picolinamide |
| B3 | 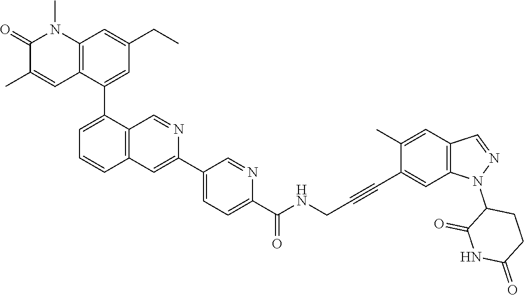 [SMILES: CCc1cc(-c2cccc3cc(-c4ccc(C(=O)NCC#Cc5cc6c(cnn6C6CCC(=O)NC6=O)cc5C)nc4)ncc23)c2cc(C)c(=O)n(C)c2c1] | N-(3-(1-(2,6-dioxopiperidin-3-yl)-5-methyl-1H- indazol-6-yl)prop-2-yn-1-yl)-5-(8-(7-ethyl-1,3- dimethyl-2-oxo-1,2-dihydroquinolin-5-yl) isoquinolin-3-yl)picolinamide |
| B4 | 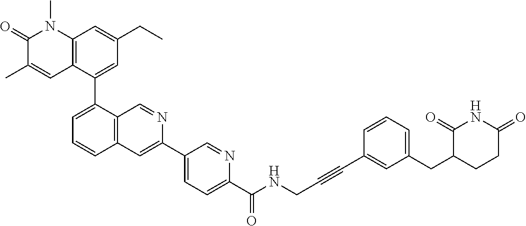 [SMILES: CCc1cc(-c2cccc3cc(-c4ccc(C(=O)NCC#Cc5cccc(CC6CCC(=O)NC6=O)c5)nc4)ncc23)c2cc(C)c(=O)n(C)c2c1] | N-(3-(3-((2,6-dioxopiperidin-3- yl)methyl)phenyl)prop-2-yn-1-yl)-5-(8-(7- ethyl-1,3-dimethyl-2-oxo-1,2-dihydroquinolin- 5-yl)isoquinolin-3-yl)picolinamide |
| B5 | 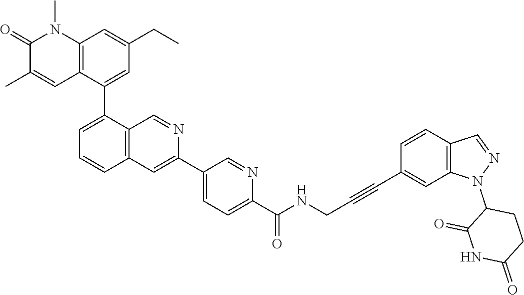 [SMILES: CCc1cc(-c2cccc3cc(-c4ccc(C(=O)NCC#Cc5ccc6cnn(C7CCC(=O)NC7=O)c6c5)nc4)ncc23)c2cc(C)c(=O)n(C)c2c1] | N-(3-(1-(2,6-dioxopiperidin-3-yl)-1H-indazol- 6-yl)prop-2-yn-1-yl)-5-(8-(7-ethyl-1,3- dimethyl-2-oxo-1,2-dihydroquinolin-5-yl) isoquinolin-3-yl)picolinamide |
| B6 | 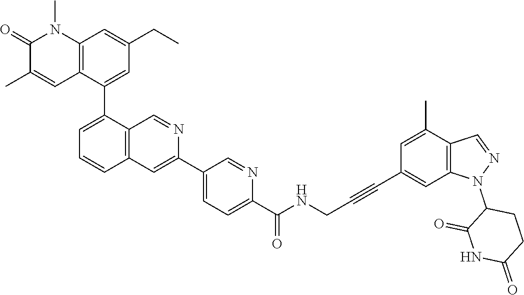 [SMILES: CCc1cc(-c2cccc3cc(-c4ccc(C(=O)NCC#Cc5cc(C)c6cnn(C7CCC(=O)NC7=O)c6c5)nc4)ncc23)c2cc(C)c(=O)n(C)c2c1] | N-(3-(1-(2,6-dioxopiperidin-3-yl)-4-methyl-1H- indazol-6-yl)prop-2-yn-1-yl)-5-(8-(7-ethyl-1,3- dimethyl-2-oxo-1,2-dihydroquinolin-5-yl) isoquinolin-3-yl)picolinamide |
| B7 | 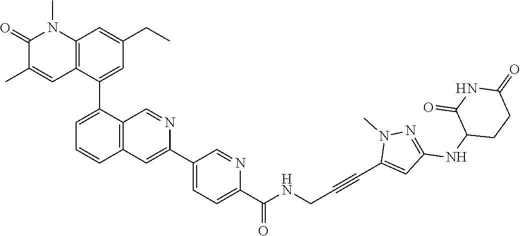 [SMILES: CCc1cc(-c2cccc3cc(-c4ccc(C(=O)NCC#Cc5cc(NC6CCC(=O)NC6=O)nn5C)nc4)ncc23)c2cc(C)c(=O)n(C)c2c1] | N-(3-(3-((2,6-dioxopiperidin-3-yl)amino)-1- methyl-1H-pyrazol-5-yl)prop-2-yn-1-yl)-5- (8-(7-ethyl-1,3-dimethyl-2-oxo-1,2- dihydroquinolin-5-yl)isoquinolin-3-yl) picolinamide |
| B8 | 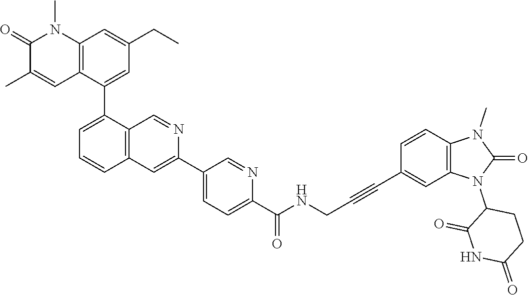 [SMILES: CCc1cc(-c2cccc3cc(-c4ccc(C(=O)NCC#Cc5ccc6c(c5)n(C5CCC(=O)NC5=O)c(=O)n6C)nc4)ncc23)c2cc(C)c(=O)n(C)c2c1] | N-(3-(3-(2,6-dioxopiperidin-3-yl)-1-methyl- 2-oxo-2,3-dihydro-1H-benzo[d]imidazol-5- yl)prop-2-yn-1-yl)-5-(8-(7-ethyl-1,3- dimethyl-2-oxo-1,2-dihydroquinolin-5-yl) isoquinolin-3-yl)picolinamide |
| B9 | 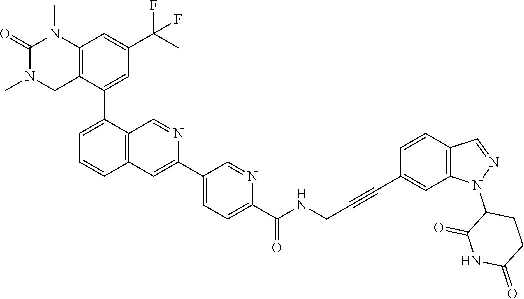 [SMILES: CN1Cc2c(-c3cccc4cc(-c5ccc(C(=O)NCC#Cc6ccc7cnn(C8CCC(=O)NC8=O)c7c6)nc5)ncc34)cc(C(C)(F)F)cc2N(C)C1=O] | 5-(8-(7-(1,1-difluoroethyl)-1,3-dimethyl-2-oxo- 1,2,3,4-tetrahydroquinazolin-5-yl)isoquinolin- 3-yl)-N-(3-(1-(2,6-dioxopiperidin-3-yl)-1H- indazol-6-yl)prop-2-yn-1-yl)picolinamide |
| B10 | 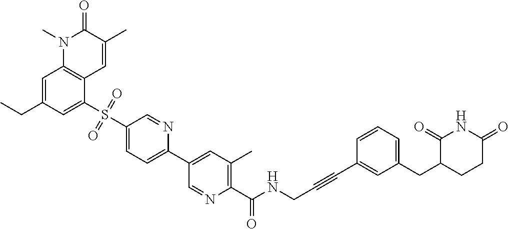 [SMILES: CCc1cc(S(=O)(=O)c2ccc(-c3cnc(C(=O)NCC#Cc4cccc(CC5CCC(=O)NC5=O)c4)c(C)c3)nc2)c2cc(C)c(=O)n(C)c2c1] | N-(3-(3-((2,6-dioxopiperidin-3-yl)amino) phenyl)prop-2-yn-1-yl)-5-((7-ethyl-1,3- dimethyl-2-oxo-1,2-dihydroquinolin-5-yl) sulfonyl)-5′-methyl-[2,3′-bipyridine]-6′- carboxamide |
| B11 | 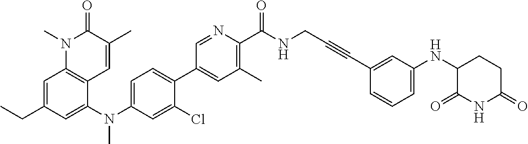 [SMILES: CCc1cc(N(C)c2ccc(-c3cnc(C(=O)NCC#Cc4cccc(NC5CCC(=O)NC5=O)c4)c(C)c3)c(Cl)c2)c2cc(C)c(=O)n(C)c2c1] | 5-(2-chloro-4-((7-ethyl-1,3-dimethyl-2-oxo- 1,2-dihydroquinolin-5-yl)(methyl)amino) phenyl)-N-(3-(3-((2,6-dioxopiperidin-3-yl) amino)phenyl)prop-2-yn-1-yl)-3- methylpicolinamide |
| B12 | 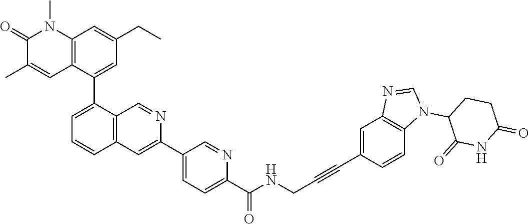 [SMILES: CCc1cc(-c2cccc3cc(-c4ccc(C(=O)NCC#Cc5ccc6c(c5)ncn6C5CCC(=O)NC5=O)nc4)ncc23)c2cc(C)c(=O)n(C)c2c1] | N-(3-(1-(2,6-dioxopiperidin-3-yl)-1H- benzo[d]imidazol-5-yl)prop-2-yn-1-yl)-5-(8- (7-ethyl-1,3-dimethyl-2-oxo-1,2-dihydro- quinolin-5-yl)isoquinolin-3-yl)picolinamide |
| B13 | 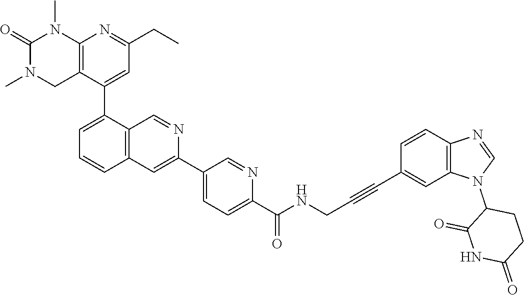 [SMILES: CCc1cc(-c2cccc3cc(-c4ccc(C(=O)NCC#Cc5ccc6ncn(C7CCC(=O)NC7=O)c6c5)nc4)ncc23)c2c(n1)N(C)C(=O)N(C)C2] | N-(3-(1-(2,6-dioxopiperidin-3-yl)-1H-indazol- 6-yl)prop-2-yn-1-yl)-5-(8-(7-ethyl-1,3- dimethyl-2-oxo-1,2,3,4-tetrahydropyrido [2,3-d]pyrimidin-5-yl)isoquinolin-3-yl) picolinamide |
| B14 | 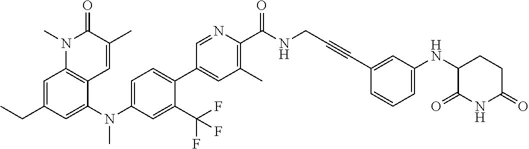 [SMILES: CCc1cc(N(C)c2ccc(-c3cnc(C(=O)NCC#Cc4cccc(NC5CCC(=O)NC5=O)c4)c(C)c3)c(C(F)(F)F)c2)c2cc(C)c(=O)n(C)c2c1] | N-(3-(3-((2,6-dioxopiperidin-3-yl)amino) phenyl)prop-2-yn-1-yl)-5-(4-((7-ethyl-1,3- dimethyl-2-oxo-1,2-dihydroquinolin-5-yl) (methyl)amino)-2-(trifluoromethyl)phenyl)- 3-methylpicolinamide |
| B15 | 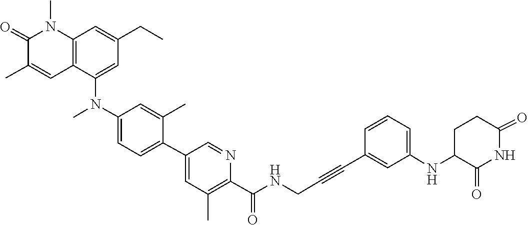 [SMILES: CCc1cc(N(C)c2ccc(-c3cnc(C(=O)NCC#Cc4cccc(NC5CCC(=O)NC5=O)c4)c(C)c3)c(C)c2)c2cc(C)c(=O)n(C)c2c1] | N-(3-(3-((2,6-dioxopiperidin-3-yl)amino) phenyl)prop-2-yn-1-yl)-5-(4-((7-ethyl-1,3- dimethyl-2-oxo-1,2-dihydroquinolin-5-yl) (methyl)amino)-2-methylphenyl)-3- methylpicolinamide |
| B16 | 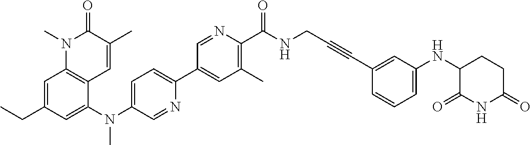 [SMILES: CCc1cc(N(C)c2ccc(-c3cnc(C(=O)NCC#Cc4cccc(NC5CCC(=O)NC5=O)c4)c(C)c3)nc2)c2cc(C)c(=O)n(C)c2c1] | N-(3-(3-((2,6-dioxopiperidin-3-yl)amino) phenyl)prop-2-yn-1-yl)-5-((7-ethyl-1,3- dimethyl-2-oxo-1,2-dihydroquinolin-5-yl) (methyl)amino)-5′-methyl-[2,3′-bipyridine]-6′- carboxamide |
| B17 | 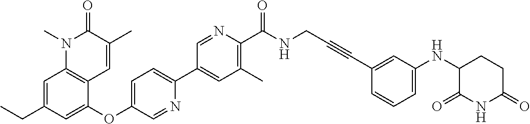 [SMILES: CCc1cc(Oc2ccc(-c3cnc(C(=O)NCC#Cc4cccc(NC5CCC(=O)NC5=O)c4)c(C)c3)nc2)c2cc(C)c(=O)n(C)c2c1] | N-(3-(3-((2,6-dioxopiperidin-3-yl)amino) phenyl)prop-2-yn-1-yl)-5-((7-ethyl-1,3- dimethyl-2-oxo-1,2-dihydroquinolin-5-yl) oxy)-5′-methyl-[2,3′-bipyridine]-6′- carboxamide |
| B18 | 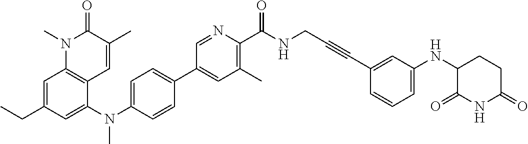 [SMILES: CCc1cc(N(C)c2ccc(-c3cnc(C(=O)NCC#Cc4cccc(NC5CCC(=O)NC5=O)c4)c(C)c3)cc2)c2cc(C)c(=O)n(C)c2c1] | N-(3-(3-((2,6-dioxopiperidin-3-yl)amino) phenyl)prop-2-yn-1-yl)-5-(4-((7-ethyl-1,3- dimethyl-2-oxo-1,2-dihydroquinolin-5-yl) (methyl)amino)phenyl)-3-methylpicolinamide |
| B19 | 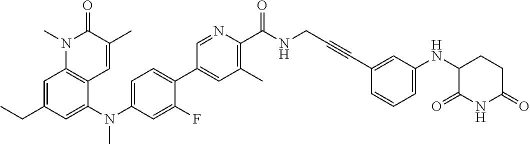 [SMILES: CCc1cc(N(C)c2ccc(-c3cnc(C(=O)NCC#Cc4cccc(NC5CCC(=O)NC5=O)c4)c(C)c3)c(F)c2)c2cc(C)c(=O)n(C)c2c1] | N-(3-(3-((2,6-dioxopiperidin-3-yl)amino) phenyl)prop-2-yn-1-yl)-5-(4-((7-ethyl-1,3- dimethyl-2-oxo-1,2-dihydroquinolin-5-yl) (methyl)amino)-2-fluorophenyl)-3- methylpicolinamide |
| B20 | 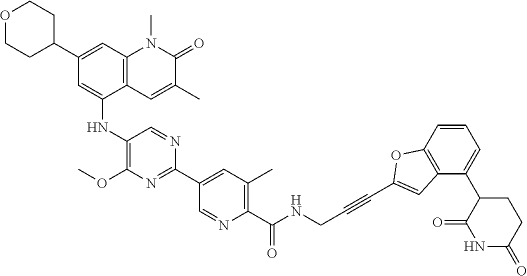 [SMILES: COc1nc(-c2cnc(C(=O)NCC#Cc3cc4c(C5CCC(=O)NC5=O)cccc4o3)c(C)c2)ncc1Nc1cc(C2CCOCC2)cc2c1cc(C)c(=O)n2C] | 5-(5-((1,3-dimethyl-2-oxo-7-(tetrahydro-2H- pyran-4-yl)-1,2-dihydroquinolin-5-yl)amino)- 4-methoxypyrimidin-2-yl)-N-(3-(4-(2,6- dioxopiperidin-3-yl)benzofuran-2-yl)prop- 2-yn-1-yl)-3-methylpicolinamide |
| B21 | 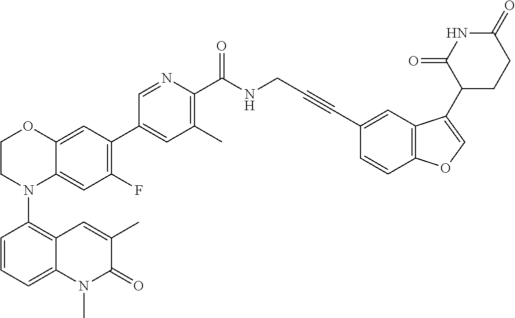 [SMILES: Cc1cc(-c2cc3c(cc2F)N(c2cccc4c2cc(C)c(=O)n4C)CCO3)cnc1C(=O)NCC#Cc1ccc2occ(C3CCC(=O)NC3=O)c2c1] | 5-(4-(1,3-dimethyl-2-oxo-1,2-dihydroquinolin- 5-yl)-6-fluoro-3,4-dihydro-2H-benzo[b][1,4] oxazin-7-yl)-N-(3-(3-(2,6-dioxopiperidin-3- yl)benzofuran-5-yl)prop-2-yn-1-yl)-3- methylpicolinamide |
| B22 | 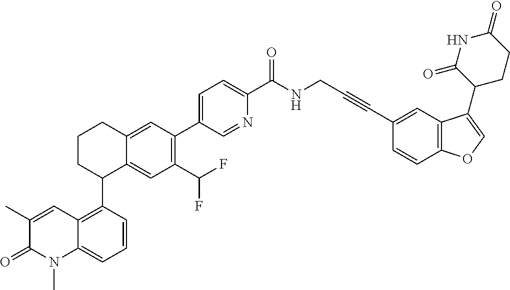 [SMILES: Cc1cc2c(C3CCCc4cc(-c5ccc(C(=O)NCC#Cc6ccc7occ(C8CCC(=O)NC8=O)c7c6)nc5)c(C(F)F)cc43)cccc2n(C)c1=O] | 5-(7-(difluoromethyl)-1′,3′-dimethyl-2′-oxo- 1′,2′,3,4-tetrahydro-2H-[1,5′-biquinolin]-6-yl)- N-(3-(3-(2,6-dioxopiperidin-3-yl)benzofuran- 5-yl)prop-2-yn-1-yl)picolinamide |
| B23 | 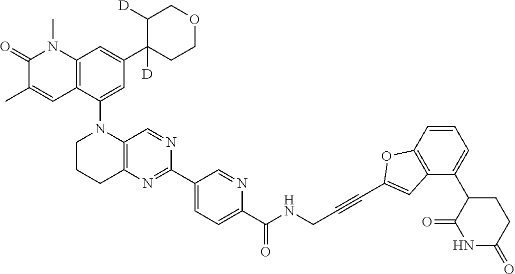 [SMILES: [2H]C1COCCC1([2H])c1cc(N2CCCc3nc(-c4ccc(C(=O)NCC#Cc5cc6c(C7CCC(=O)NC7=O)cccc6o5)nc4)ncc32)c2cc(C)c(=O)n(C)c2c1] | 5-(5-(1,3-dimethyl-2-oxo-7-(tetrahydro-2H- pyran-4-yl-3,4-d2)-1,2-dihydroquinolin-5-yl)- 5,6,7,8-tetrahydropyrido[3,2-d]pyrimidin-2- yl)-N-(3-(4-(2,6-dioxopiperidin-3-yl) benzofuran-2-yl)prop-2-yn-1-yl)picolinamide |
| B24 | 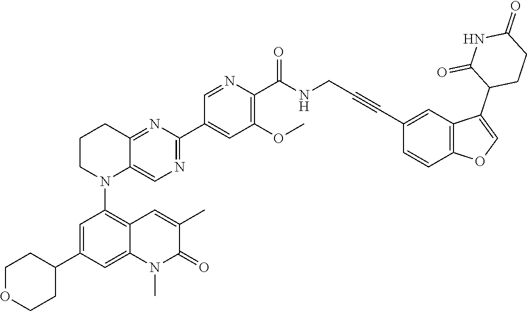 [SMILES: COc1cc(-c2ncc3c(n2)CCCN3c2cc(C3CCOCC3)cc3c2cc(C)c(=O)n3C)cnc1C(=O)NCC#Cc1ccc2occ(C3CCC(=O)NC3=O)c2c1] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (3-(2,6-dioxopiperidin-3-yl)benzofuran-5- yl)prop-2-yn-1-yl)-3-methoxypicolinamide |
| B25 | 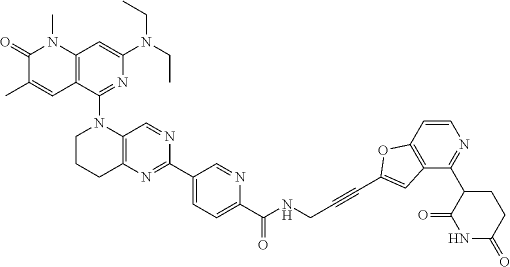 [SMILES: CCN(CC)c1cc2c(cc(C)c(=O)n2C)c(N2CCCc3nc(-c4ccc(C(=O)NCC#Cc5cc6c(C7CCC(=O)NC7=O)nccc6o5)nc4)ncc32)n1] | 5-(5-(7-(diethylamino)-1,3-dimethyl-2-oxo-1,2- dihydro-1,6-naphthyridin-5-yl)-5,6,7,8- tetrahydropyrido[3,2-d]pyrimidin-2-yl)-N-(3- (4-(2,6-dioxopiperidin-3-yl)furo[3,2-c]pyridin- 2-yl)prop-2-yn-1-yl)picolinamide |
| B26 | 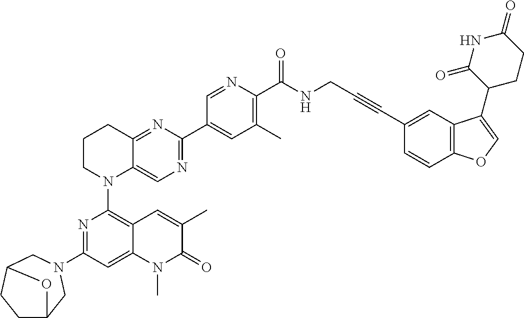 [SMILES: Cc1cc(-c2ncc3c(n2)CCCN3c2nc(N3CC4CCC(C3)O4)cc3c2cc(C)c(=O)n3C)cnc1C(=O)NCC#Cc1ccc2occ(C3CCC(=O)NC3=O)c2c1] | 5-(5-(7-(8-oxa-3-azabicyclo[3.2.1]octan-3-yl)- 1,3-dimethyl-2-oxo-1,2-dihydro-1,6- naphthyridin-5-yl)-5,6,7,8-tetrahydropyrido [3,2-d]pyrimidin-2-yl)-N-(3-(3-(2,6- dioxopiperidin-3-yl)benzofuran-5-yl)prop-2- yn-1-yl)-3-methylpicolinamide |
| B27 | 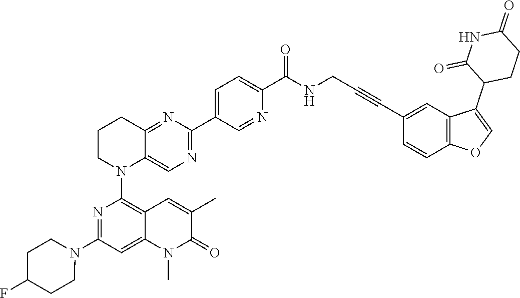 [SMILES: Cc1cc2c(N3CCCc4nc(-c5ccc(C(=O)NCC#Cc6ccc7occ(C8CCC(=O)NC8=O)c7c6)nc5)ncc43)nc(N3CCC(F)CC3)cc2n(C)c1=O] | N-(3-(3-(2,6-dioxopiperidin-3-yl)benzofuran- 5-yl)prop-2-yn-1-yl)-5-(5-(7-(4-fluoro- piperidin-1-yl)-1,3-dimethyl-2-oxo-1,2- dihydro-1,6-naphthyridin-5-yl)-5,6,7,8- tetrahydropyrido[3,2-d]pyrimidin-2-yl) picolinamide |
| B28 | 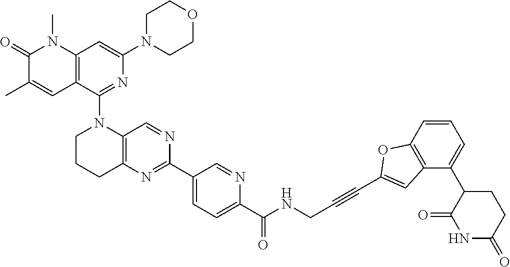 [SMILES: Cc1cc2c(N3CCCc4nc(-c5ccc(C(=O)NCC#Cc6cc7c(C8CCC(=O)NC8=O)cccc7o6)nc5)ncc43)nc(N3CCOCC3)cc2n(C)c1=O] | 5-(5-(1,3-dimethyl-7-morpholino-2-oxo-1,2- dihydro-1,6-naphthyridin-5-yl)-5,6,7,8- tetrahydropyrido[3,2-d]pyrimidin-2-yl)-N-(3- (4-(2,6-dioxopiperidin-3-yl)benzofuran-2- yl)prop-2-yn-1-yl)picolinamide |
| B29 | 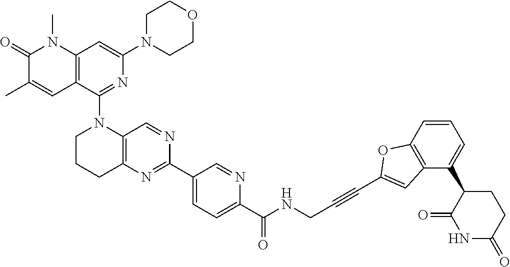 [SMILES: Cc1cc2c(N3CCCc4nc(-c5ccc(C(=O)NCC#Cc6cc7c([C@H]8CCC(=O)NC8=O)cccc7o6)nc5)ncc43)nc(N3CCOCC3)cc2n(C)c1=O] | (R)-5-(5-(1,3-dimethyl-7-morpholino-2-oxo- 1,2-dihydro-1,6-naphthyridin-5-yl)-5,6,7,8- tetrahydropyrido[3,2-d]pyrimidin-2-yl)-N-(3- (4-(2,6-dioxopiperidin-3-yl)benzofuran-2-yl) prop-2-yn-1-yl)picolinamide |
| B30 | 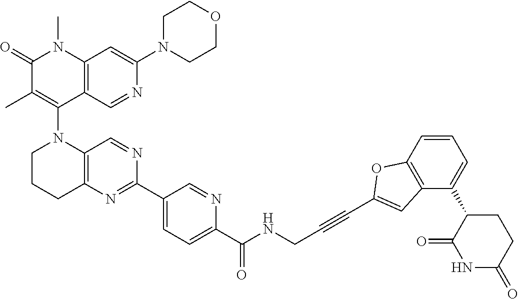 [SMILES: Cc1c(N2CCCc3nc(-c4ccc(C(=O)NCC#Cc5cc6c([C@@H]7CCC(=O)NC7=O)cccc6o5)nc4)ncc32)c2cnc(N3CCOCC3)cc2n(C)c1=O] | (S)-5-(5-(1,3-dimethyl-7-morpholino-2-oxo- 1,2-dihydro-1,6-naphthyridin-5-yl)-5,6,7,8- tetrahydropyrido[3,2-d]pyrimidin-2-yl)-N-(3- (4-(2,6-dioxopiperidin-3-yl)benzofuran-2-yl) prop-2-yn-1-yl)picolinamide |
| B31 | 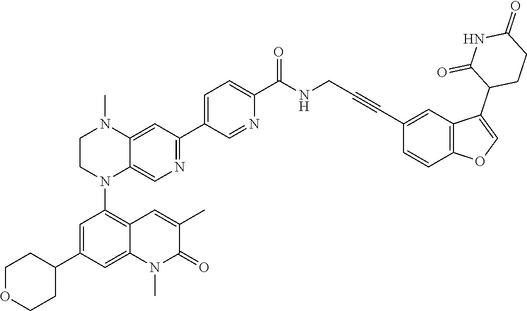 [SMILES: Cc1cc2c(N3CCN(C)c4cc(-c5ccc(C(=O)NCC#Cc6ccc7occ(C8CCC(=O)NC8=O)c7c6)nc5)ncc43)cc(C3CCOCC3)cc2n(C)c1=O] | 5-(4-(1,3-dimethyl-2-oxo-7-(tetrahydro-2H- pyran-4-yl)-1,2-dihydroquinolin-5-yl)-1- methyl-1,2,3,4-tetrahydropyrido[3,4-b] pyrazin-7-yl)-N-(3-(3-(2,6-dioxopiperidin- 3-yl)benzofuran-5-yl)prop-2-yn-1-yl) picolinamide |
| B32 | 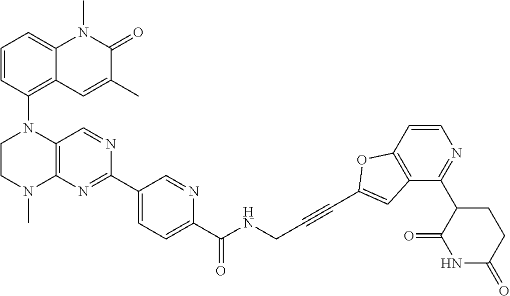 [SMILES: Cc1cc2c(N3CCN(C)c4nc(-c5ccc(C(=O)NCC#Cc6cc7c(C8CCC(=O)NC8=O)nccc7o6)nc5)ncc43)cccc2n(C)c1=O] | 5-(5-(1,3-dimethyl-2-oxo-1,2-dihydroquinolin- 5-yl)-8-methyl-5,6,7,8-tetrahydropteridin-2- yl)-N-(3-(4-(2,6-dioxopiperidin-3-yl)furo[3,2-c] pyridin-2-yl)prop-2-yn-1-yl)picolinamide |
| B33 | 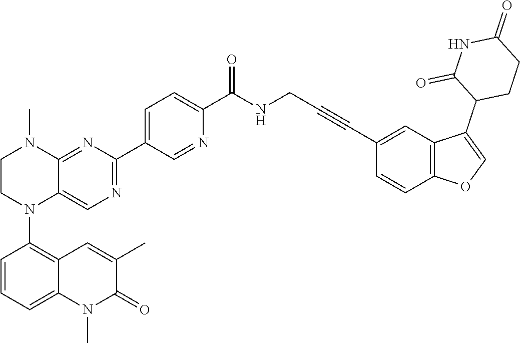 [SMILES: Cc1cc2c(N3CCN(C)c4nc(-c5ccc(C(=O)NCC#Cc6ccc7occ(C8CCC(=O)NC8=O)c7c6)nc5)ncc43)cccc2n(C)c1=O] | 5-(5-(1,3-dimethyl-2-oxo-1,2-dihydroquinolin- 5-yl)-8-methyl-5,6,7,8-tetrahydropteridin-2-yl)- N-(3-(3-(2,6-dioxopiperidin-3-yl)benzofuran- 5-yl)prop-2-yn-1-yl)picolinamide |
| B34 | 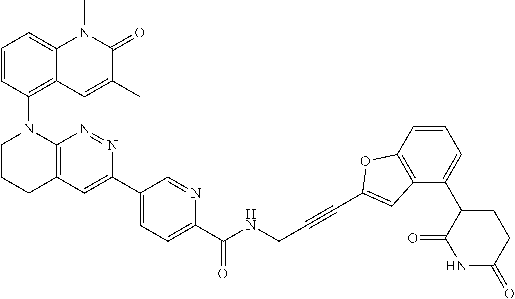 [SMILES: Cc1cc2c(N3CCCc4cc(-c5ccc(C(=O)NCC#Cc6cc7c(C8CCC(=O)NC8=O)cccc7o6)nc5)nnc43)cccc2n(C)c1=O] | 5-(8-(1,3-dimethyl-2-oxo-1,2-dihydroquinolin- 5-yl)-5,6,7,8-tetrahydropyrido[2,3-c]pyridazin- 3-yl)-N-(3-(4-(2,6-dioxopiperidin-3-yl) benzofuran-2-yl)prop-2-yn-1-yl)picolinamide |
| B35 | 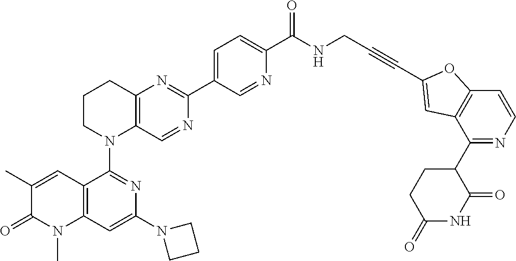 [SMILES: Cc1cc2c(N3CCCc4nc(-c5ccc(C(=O)NCC#Cc6cc7c(C8CCC(=O)NC8=O)nccc7o6)nc5)ncc43)nc(N3CCC3)cc2n(C)c1=O] | 5-(5-(7-(azetidin-1-yl)-1,3-dimethyl-2-oxo-1,2- dihydro-1,6-naphthyridin-5-yl)-5,6,7,8- tetrahydropyrido[3,2-d]pyrimidin-2-yl)-N-(3- (4-(2,6-dioxopiperidin-3-yl)furo[3,2-c]pyridin- 2-yl)prop-2-yn-1-yl)picolinamide |
| B36 | 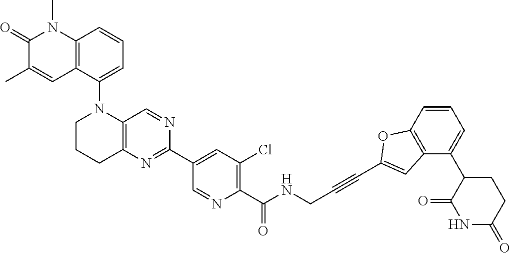 [SMILES: Cc1cc2c(N3CCCc4nc(-c5cnc(C(=O)NCC#Cc6cc7c(C8CCC(=O)NC8=O)cccc7o6)c(Cl)c5)ncc43)cccc2n(C)c1=O] | 3-chloro-5-(5-(1,3-dimethyl-2-oxo-1,2- dihydroquinolin-5-yl)-5,6,7,8-tetrahydropyrido [3,2-d]pyrimidin-2-yl)-N-(3-(4-(2,6-dioxo- piperidin-3-yl)benzofuran-2-yl)prop-2-yn-1- yl)picolinamide |
| B37 | 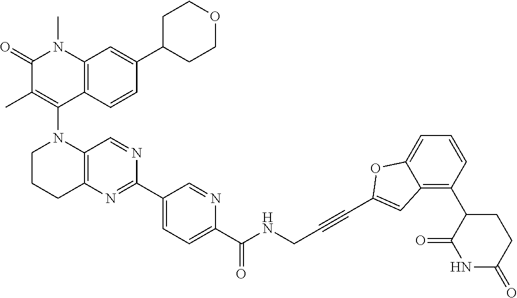 [SMILES: Cc1c(N2CCCc3nc(-c4ccc(C(=O)NCC#Cc5cc6c(C7CCC(=O)NC7=O)cccc6o5)nc4)ncc32)c2ccc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,6-dioxopiperidin-3-yl)benzofuran-2-yl) prop-2-yn-1-yl)picolinamide |
| B38 | 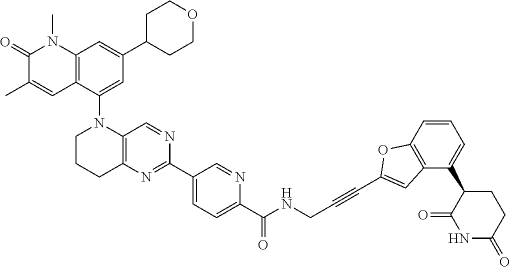 [SMILES: Cc1cc2c(N3CCCc4nc(-c5ccc(C(=O)NCC#Cc6cc7c([C@H]8CCC(=O)NC8=O)cccc7o6)nc5)ncc43)cc(C3CCOCC3)cc2n(C)c1=O] | (R)-5-(5-(1,3-dimethyl-2-oxo-7-(tetrahydro- 2H-pyran-4-yl)-1,2-dihydroquinolin-5-yl)- 5,6,7,8-tetrahydropyrido[3,2-d]pyrimidin-2-yl)- N-(3-(4-(2,6-dioxopiperidin-3-yl)benzofuran- 2-yl)prop-2-yn-1-yl)picolinamide |
| B39 | 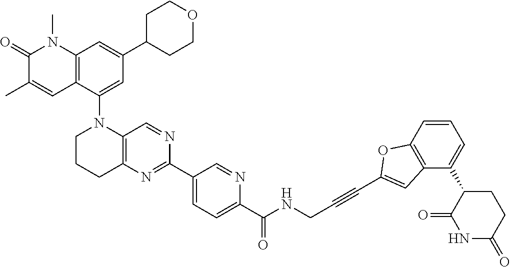 [SMILES: Cc1cc2c(N3CCCc4nc(-c5ccc(C(=O)NCC#Cc6cc7c([C@@H]8CCC(=O)NC8=O)cccc7o6)nc5)ncc43)cc(C3CCOCC3)cc2n(C)c1=O] | (S)-5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,6-dioxopiperidin-3-yl)benzofuran-2-yl) prop-2-yn-1-yl)picolinamide |
| B40 | 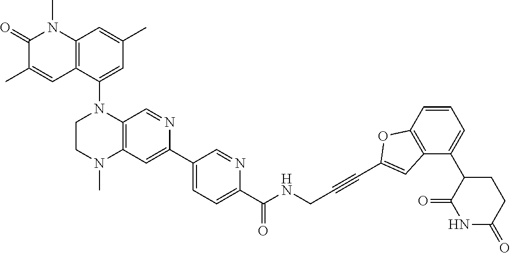 [SMILES: Cc1cc(N2CCN(C)c3cc(-c4ccc(C(=O)NCC#Cc5cc6c(C7CCC(=O)NC7=O)cccc6o5)nc4)ncc32)c2cc(C)c(=O)n(C)c2c1] | N-(3-(4-(2,6-dioxopiperidin-3-yl)benzofuran- 2-yl)prop-2-yn-1-yl)-5-(1-methyl-4-(1,3,7- trimethyl-2-oxo-1,2-dihydroquinolin-5-yl)- 1,2,3,4-tetrahydropyrido[3,4-b]pyrazin-7-yl) picolinamide |
| B41 | 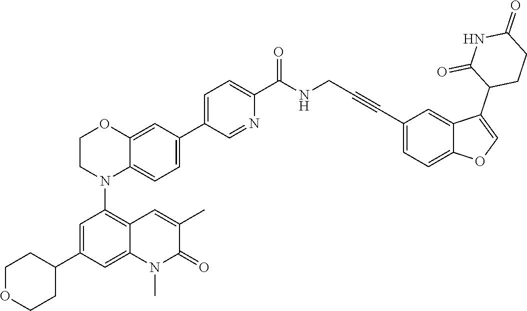 [SMILES: Cc1cc2c(N3CCOc4cc(-c5ccc(C(=O)NCC#Cc6ccc7occ(C8CCC(=O)NC8=O)c7c6)nc5)ccc43)cc(C3CCOCC3)cc2n(C)c1=O] | 5-(4-(1,3-dimethyl-2-oxo-7-(tetrahydro-2H- pyran-4-yl)-1,2-dihydroquinolin-5-yl)-3,4- dihydro-2H-benzo[b][1,4]oxazin-7-yl)-N-(3- (3-(2,6-dioxopiperidin-3-yl)benzofuran-5-yl) prop-2-yn-1-yl)picolinamide |
| B42 | 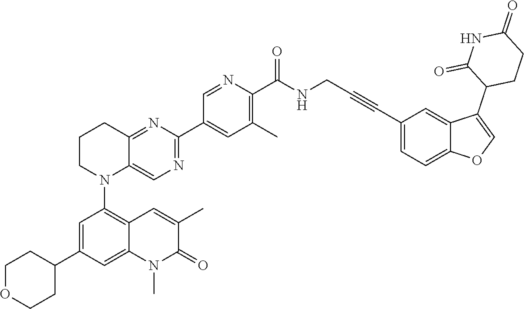 [SMILES: Cc1cc(-c2ncc3c(n2)CCCN3c2cc(C3CCOCC3)cc3c2cc(C)c(=O)n3C)cnc1C(=O)NCC#Cc1ccc2occ(C3CCC(=O)NC3=O)c2c1] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (3-(2,6-dioxopiperidin-3-yl)benzofuran-5-yl) prop-2-yn-1-yl)-3-methylpicolinamide |
| B43 | 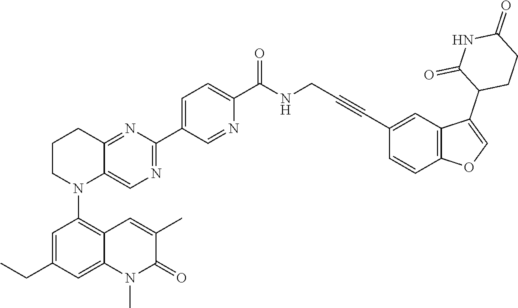 [SMILES: CCc1cc(N2CCCc3nc(-c4ccc(C(=O)NCC#Cc5ccc6occ(C7CCC(=O)NC7=O)c6c5)nc4)ncc32)c2cc(C)c(=O)n(C)c2c1] | N-(3-(3-(2,6-dioxopiperidin-3-yl)benzofuran-5- yl)prop-2-yn-1-yl)-5-(5-(7-ethyl-1,3-dimethyl- 2-oxo-1,2-dihydroquinolin-5-yl)-5,6,7,8- tetrahydropyrido[3,2-d]pyrimidin-2-yl) picolinamide |
| B44 | 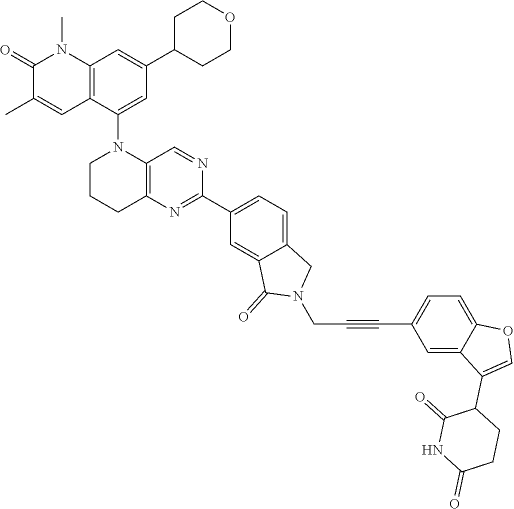 [SMILES: Cc1cc2c(N3CCCc4nc(-c5ccc6c(c5)C(=O)N(CC#Cc5ccc7occ(C8CCC(=O)NC8=O)c7c5)C6)ncc43)cc(C3CCOCC3)cc2n(C)c1=O] | 3-(5-(3-(6-(5-(1,3-dimethyl-2-oxo-7- (tetrahydro-2H-pyran-4-yl)-1,2-dihydro- quinolin-5-yl)-5,6,7,8-tetrahydropyrido[3,2-d] pyrimidin-2-yl)-1-oxoisoindolin-2-yl)prop-1- yn-1-yl)benzofuran-3-yl)piperidine-2,6-dione |
| B45 | 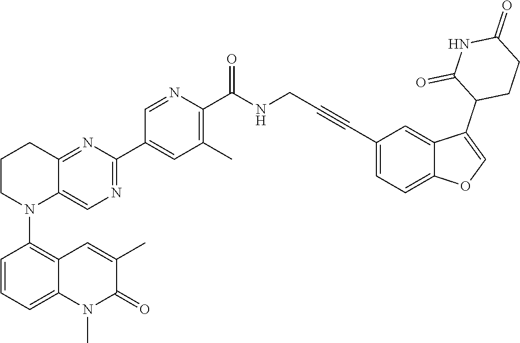 [SMILES: Cc1cc(-c2ncc3c(n2)CCCN3c2cccc3c2cc(C)c(=O)n3C)cnc1C(=O)NCC#Cc1ccc2occ(C3CCC(=O)NC3=O)c2c1] | 5-(5-(1,3-dimethyl-2-oxo-1,2-dihydroquinolin- 5-yl)-5,6,7,8-tetrahydropyrido[3,2-d]pyrimidin- 2-yl)-N-(3-(3-(2,6-dioxopiperidin-3-yl) benzofuran-5-yl)prop-2-yn-1-yl)-3- methylpicolinamide |
| B46 | 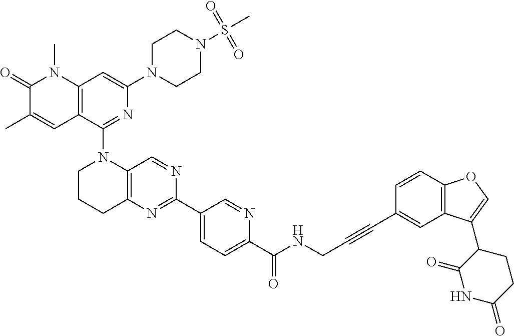 [SMILES: Cc1cc2c(N3CCCc4nc(-c5ccc(C(=O)NCC#Cc6ccc7occ(C8CCC(=O)NC8=O)c7c6)nc5)ncc43)nc(N3CCN(S(C)(=O)=O)CC3)cc2n(C)c1=O] | 5-(5-(1,3-dimethyl-7-(4-(methylsulfonyl) piperazin-1-yl)-2-oxo-1,2-dihydro-1,6- naphthyridin-5-yl)-5,6,7,8-tetrahydropyrido [3,2-d]pyrimidin-2-yl)-N-(3-(3-(2,6- dioxopiperidin-3-yl)benzofuran-5-yl)prop-2- yn-1-yl)picolinamide |
| B47 | 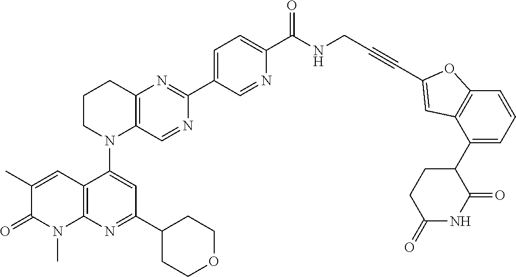 [SMILES: Cc1cc2c(N3CCCc4nc(-c5ccc(C(=O)NCC#Cc6cc7c(C8CCC(=O)NC8=O)cccc7o6)nc5)ncc43)cc(C3CCOCC3)nc2n(C)c1=O] | 5-(5-(6,8-dimethyl-7-oxo-2-(tetrahydro-2H- pyran-4-yl)-7,8-dihydro-1,8-naphthyridin-4- yl)-5,6,7,8-tetrahydropyrido[3,2-d]pyrimidin- 2-yl)-N-(3-(4-(2,6-dioxopiperidin-3-yl) benzofuran-2-yl)prop-2-yn-1-yl)picolinamide |
| B48 | 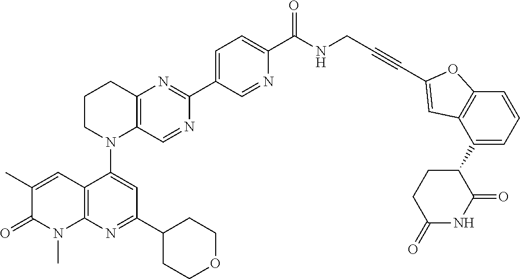 [SMILES: Cc1cc2c(N3CCCc4nc(-c5ccc(C(=O)NCC#Cc6cc7c([C@H]8CCC(=O)NC8=O)cccc7o6)nc5)ncc43)cc(C3CCOCC3)nc2n(C)c1=O] | (R)-5-(5-(6,8-dimethyl-7-oxo-2-(tetrahydro-2H- pyran-4-yl)-7,8-dihydro-1,8-naphthyridin-4- yl)-5,6,7,8-tetrahydropyrido[3,2-d]pyrimidin- 2-yl)-N-(3-(4-(2,6-dioxopiperidin-3-yl) benzofuran-2-yl)prop-2-yn-1-yl)picolinamide |
| B49 | 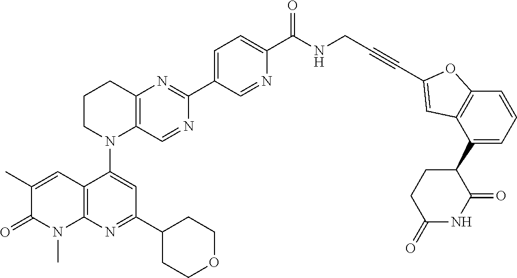 [SMILES: Cc1cc2c(N3CCCc4nc(-c5ccc(C(=O)NCC#Cc6cc7c([C@@H]8CCC(=O)NC8=O)cccc7o6)nc5)ncc43)cc(C3CCOCC3)nc2n(C)c1=O] | (S)-5-(5-(6,8-dimethyl-7-oxo-2-(tetrahydro-2H- pyran-4-yl)-7,8-dihydro-1,8-naphthyridin-4- yl)-5,6,7,8-tetrahydropyrido[3,2-d]pyrimidin- 2-yl)-N-(3-(4-(2,6-dioxopiperidin-3-yl) benzofuran-2-yl)prop-2-yn-1-yl)picolinamide |
| B50 | 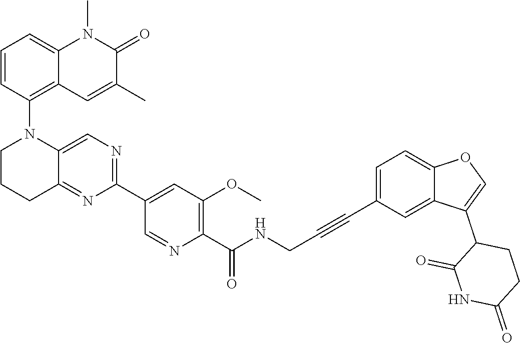 [SMILES: COc1cc(-c2ncc3c(n2)CCCN3c2cccc3c2cc(C)c(=O)n3C)cnc1C(=O)NCC#Cc1ccc2occ(C3CCC(=O)NC3=O)c2c1] | 5-(5-(1,3-dimethyl-2-oxo-1,2-dihydroquinolin- 5-yl)-5,6,7,8-tetrahydropyrido[3,2-d]pyrimidin- 2-yl)-N-(3-(3-(2,6-dioxopiperidin-3-yl) benzofuran-5-yl)prop-2-yn-1-yl)-3- methoxypicolinamide |
| B51 | 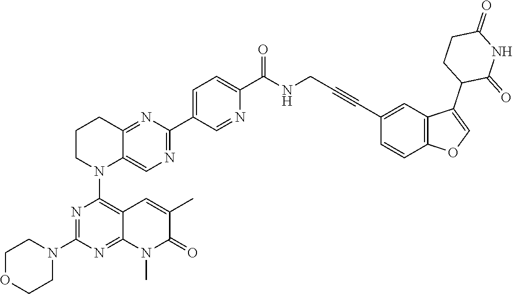 [SMILES: Cc1cc2c(N3CCCc4nc(-c5ccc(C(=O)NCC#Cc6ccc7occ(C8CCC(=O)NC8=O)c7c6)nc5)ncc43)nc(N3CCOCC3)nc2n(C)c1=O] | 5-(5-(6,8-dimethyl-2-morpholino-7-oxo-7,8- dihydropyrido[2,3-d]pyrimidin-4-yl)-5,6,7,8- tetrahydropyrido[3,2-d]pyrimidin-2-yl)-N-(3- (3-(2,6-dioxopiperidin-3-yl)benzofuran-5-yl) prop-2-yn-1-yl)picolinamide |
| B52 | 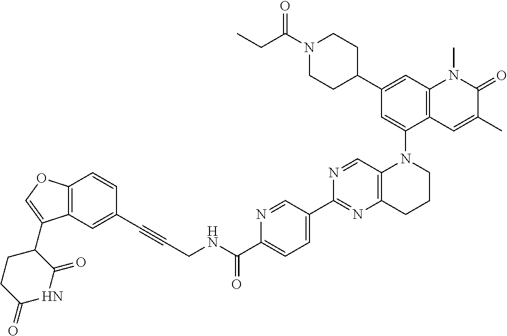 [SMILES: CCC(=O)N1CCC(c2cc(N3CCCc4nc(-c5ccc(C(=O)NCC#Cc6ccc7occ(C8CCC(=O)NC8=O)c7c6)nc5)ncc43)c3cc(C)c(=O)n(C)c3c2)CC1] | 5-(5-(1,3-dimethyl-2-oxo-7-(1-propionyl- piperidin-4-yl)-1,2-dihydroquinolin-5-yl)-5,6, 7,8-tetrahydropyrido[3,2-d]pyrimidin-2-yl)-N- (3-(3-(2,6-dioxopiperidin-3-yl)benzofuran- 5-yl)prop-2-yn-1-yl)picolinamide |
| B53 | 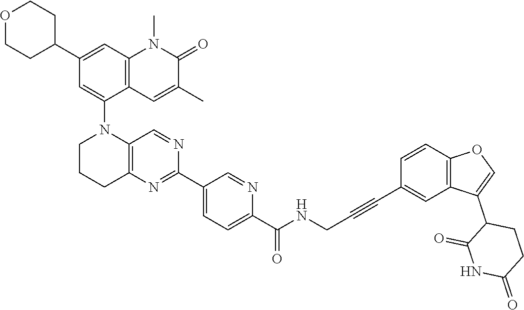 [SMILES: Cc1cc2c(N3CCCc4nc(-c5ccc(C(=O)NCC#Cc6ccc7occ(C8CCC(=O)NC8=O)c7c6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (3-(2,6-dioxopiperidin-3-yl)benzofuran-5-yl) prop-2-yn-1-yl)picolinamide |
| B54 | 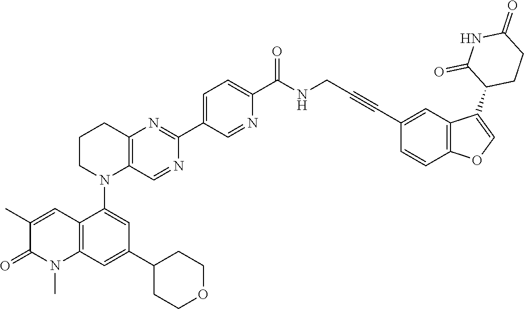 [SMILES: Cc1cc2c(N3CCCc4nc(-c5ccc(C(=O)NCC#Cc6ccc7occ([C@H]8CCC(=O)NC8=O)c7c6)nc5)ncc43)cc(C3CCOCC3)cc2n(C)c1=O] | (R)-5-(5-(1,3-dimethyl-2-oxo-7-(tetrahydro- 2H-pyran-4-yl)-1,2-dihydroquinolin-5-yl)- 5,6,7,8-tetrahydropyrido[3,2-d]pyrimidin-2-yl)- N-(3-(3-(2,6-dioxopiperidin-3-yl)benzofuran- 5-yl)prop-2-yn-1-yl)picolinamide |
| B55 | 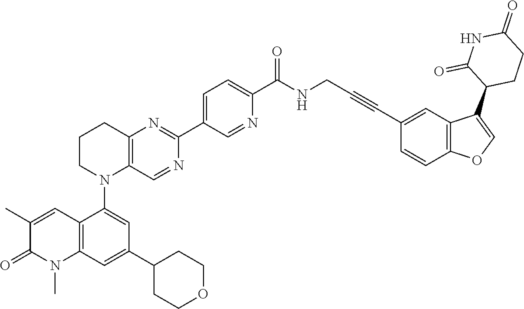 [SMILES: Cc1cc2c(N3CCCc4nc(-c5ccc(C(=O)NCC#Cc6ccc7occ([C@@H]8CCC(=O)NC8=O)c7c6)nc5)ncc43)cc(C3CCOCC3)cc2n(C)c1=O] | (S)-5-(5-(1,3-dimethyl-2-oxo-7-(tetrahydro-2H- pyran-4-yl)-1,2-dihydroquinolin-5-yl)- 5,6,7,8-tetrahydropyrido[3,2-d]pyrimidin-2- yl)-N-(3-(3-(2,6-dioxopiperidin-3-yl) benzofuran-5-yl)prop-2-yn-1-yl)picolinamide |
| B56 | 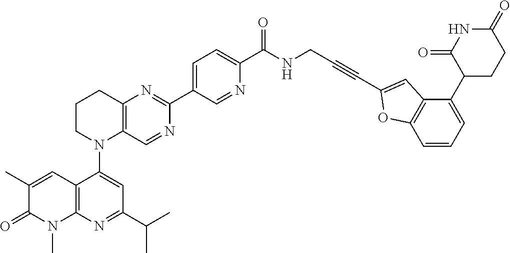 [SMILES: Cc1cc2c(N3CCCc4nc(-c5ccc(C(=O)NCC#Cc6cc7c(C8CCC(=O)NC8=O)cccc7o6)nc5)ncc43)cc(C(C)C)nc2n(C)c1=O] | N-(3-(4-(2,6-dioxopiperidin-3-yl)benzofuran- 2-yl)prop-2-yn-1-yl)-5-(5-(2-isopropyl-6,8- dimethyl-7-oxo-7,8-dihydro-1,8-naphthyridin- 4-yl)-5,6,7,8-tetrahydropyrido[3,2-d] pyrimidin-2-yl)picolinamide |
| B57 | 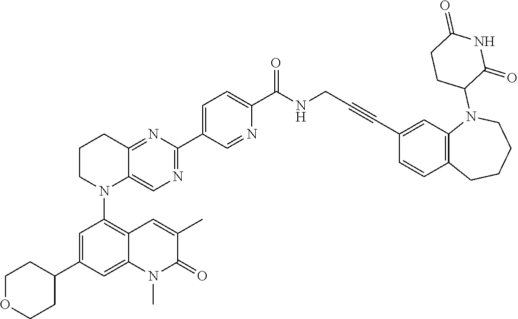 [SMILES: Cc1cc2c(N3CCCc4nc(-c5ccc(C(=O)NCC#Cc6ccc7c(c6)N(C6CCC(=O)NC6=O)CCCC7)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (1-(2,6-dioxopiperidin-3-yl)-2,3,4,5-tetrahydro- 1H-benzo[b]azepin-8-yl)prop-2-yn-1-yl) picolinamide |
| B58 | 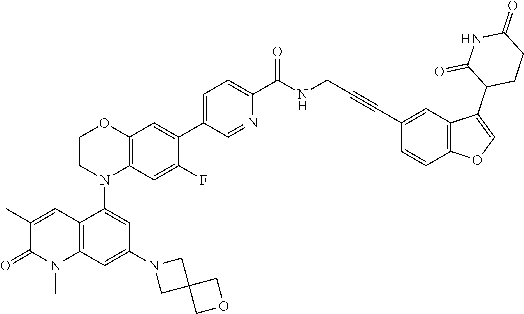 [SMILES: Cc1cc2c(N3CCOc4cc(-c5ccc(C(=O)NCC#Cc6ccc7occ(C8CCC(=O)NC8=O)c7c6)nc5)c(F)cc43)cc(N3CC4(COC4)C3)cc2n(C)c1=O] | 5-(4-(1,3-dimethyl-2-oxo-7-(2-oxa-6- azaspiro[3.3]heptan-6-yl)-1,2-dihydro- quinolin-5-yl)-6-fluoro-3,4-dihydro-2H- benzo[b][1,4]oxazin-7-yl)-N-(3-(3-(2,6- dioxopiperidin-3-yl)benzofuran-5-yl)prop- 2-yn-1-yl)picolinamide |
| B59 | 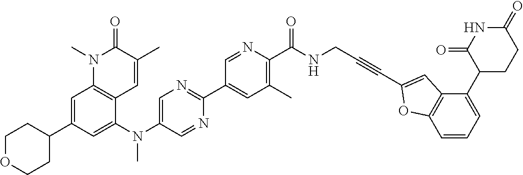 [SMILES: Cc1cc(-c2ncc(N(C)c3cc(C4CCOCC4)cc4c3cc(C)c(=O)n4C)cn2)cnc1C(=O)NCC#Cc1cc2c(C3CCC(=O)NC3=O)cccc2o1] | 5-(5-((1,3-dimethyl-2-oxo-7-(tetrahydro-2H- pyran-4-yl)-1,2-dihydroquinolin-5-yl) (methyl)amino)pyrimidin-2-yl)-N-(3-(4-(2,6- dioxopiperidin-3-yl)benzofuran-2-yl)prop-2- yn-1-yl)-3-methylpicolinamide |
| B60 | 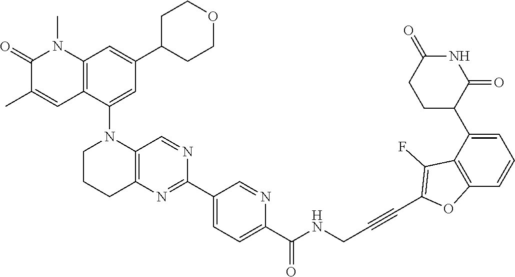 [SMILES: Cc1cc2c(N3CCCc4nc(-c5ccc(C(=O)NCC#Cc6oc7cccc(C8CCC(=O)NC8=O)c7c6F)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,6-dioxopiperidin-3-yl)-3-fluorobenzo- furan-2-yl)prop-2-yn-1-yl)picolinamide |
| B61 | 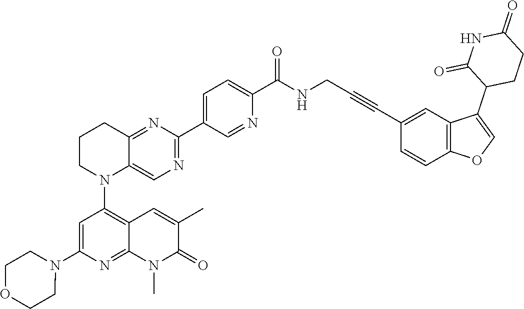 [SMILES: Cc1cc2c(N3CCCc4nc(-c5ccc(C(=O)NCC#Cc6ccc7occ(C8CCC(=O)NC8=O)c7c6)nc5)ncc43)cc(N3CCOCC3)nc2n(C)c1=O] | 5-(5-(6,8-dimethyl-2-morpholino-7-oxo-7,8- dihydro-1,8-naphthyridin-4-yl)-5,6,7,8- tetrahydropyrido[3,2-d]pyrimidin-2-yl)-N-(3- (3-(2,6-dioxopiperidin-3-yl)benzofuran-5- yl)prop-2-yn-1-yl)picolinamide |
| B62 | 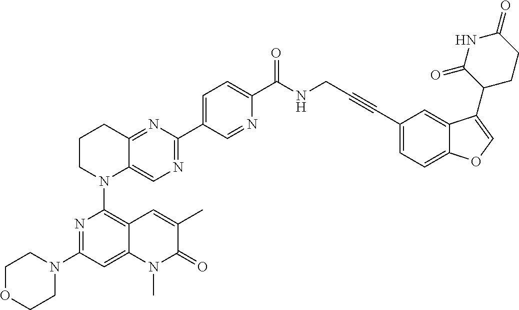 [SMILES: Cc1cc2c(N3CCCc4nc(-c5ccc(C(=O)NCC#Cc6ccc7occ(C8CCC(=O)NC8=O)c7c6)nc5)ncc43)nc(N3CCOCC3)cc2n(C)c1=O] | 5-(5-(1,3-dimethyl-7-morpholino-2-oxo-1,2- dihydro-1,6-naphthyridin-5-yl)-5,6,7,8- tetrahydropyrido[3,2-d]pyrimidin-2-yl)-N-(3- (3-(2,6-dioxopiperidin-3-yl)benzofuran-5- yl)prop-2-yn-1-yl)picolinamide |
| B63 | 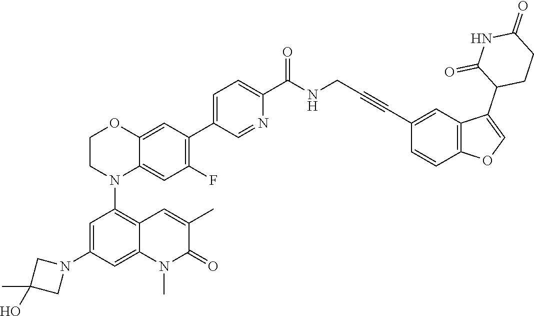 [SMILES: Cc1cc2c(N3CCOc4cc(-c5ccc(C(=O)NCC#Cc6ccc7occ(C8CCC(=O)NC8=O)c7c6)nc5)c(F)cc43)cc(N3CC(C)(O)C3)cc2n(C)c1=O] | N-(3-(3-(2,6-dioxopiperidin-3-yl)benzofuran- 5-yl)prop-2-yn-1-yl)-5-(6-fluoro-4-(7-(3- hydroxy-3-methylazetidin-1-yl)-1,3-dimethyl- 2-oxo-1,2-dihydroquinolin-5-yl)-3,4- dihydro-2H-benzo[b][1,4]oxazin-7-yl) picolinamide |
| B64 | 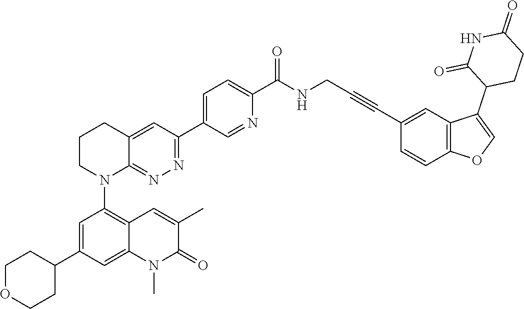 [SMILES: Cc1cc2c(N3CCCc4cc(-c5ccc(C(=O)NCC#Cc6ccc7occ(C8CCC(=O)NC8=O)c7c6)nc5)nnc43)cc(C3CCOCC3)cc2n(C)c1=O] | 5-(8-(1,3-dimethyl-2-oxo-7-(tetrahydro-2H- pyran-4-yl)-1,2-dihydroquinolin-5-yl)-5,6,7,8- tetrahydropyrido[2,3-c]pyridazin-3-yl)-N-(3- (3-(2,6-dioxopiperidin-3-yl)benzofuran-5-yl) prop-2-yn-1-yl)picolinamide |
| B65 | 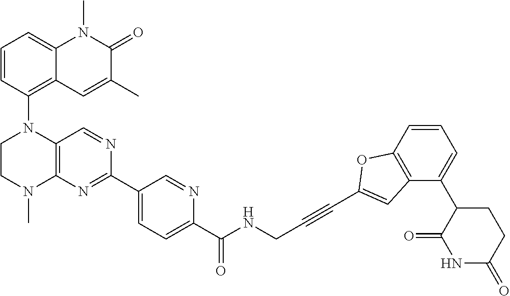 [SMILES: Cc1cc2c(N3CCN(C)c4nc(-c5ccc(C(=O)NCC#Cc6cc7c(C8CCC(=O)NC8=O)cccc7o6)nc5)ncc43)cccc2n(C)c1=O] | 5-(5-(1,3-dimethyl-2-oxo-1,2-dihydroquinolin- 5-yl)-8-methyl-5,6,7,8-tetrahydropteridin-2- yl)-N-(3-(4-(2,6-dioxopiperidin-3-yl) benzofuran-2-yl)prop-2-yn-1-yl)picolinamide |
| B66 | 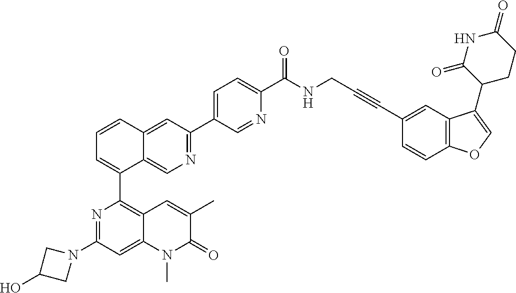 [SMILES: Cc1cc2c(-c3cccc4cc(-c5ccc(C(=O)NCC#Cc6ccc7occ(C8CCC(=O)NC8=O)c7c6)nc5)ncc34)nc(N3CC(O)C3)cc2n(C)c1=O] | N-(3-(3-(2,6-dioxopiperidin-3-yl)benzofuran- 5-yl)prop-2-yn-1-yl)-5-(8-(7-(3-hydroxy- azetidin-1-yl)-1,3-dimethyl-2-oxo-1,2-dihydro- 1,6-naphthyridin-5-yl)isoquinolin-3-yl) picolinamide |
| B67 | 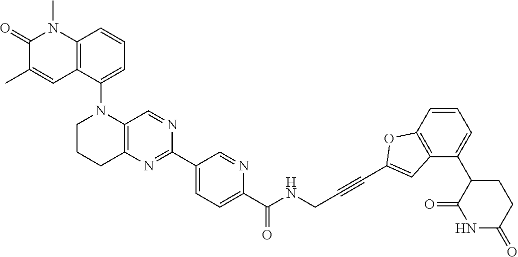 [SMILES: Cc1cc2c(N3CCCc4nc(-c5ccc(C(=O)NCC#Cc6cc7c(C8CCC(=O)NC8=O)cccc7o6)nc5)ncc43)cccc2n(C)c1=O] | 5-(5-(1,3-dimethyl-2-oxo-1,2-dihydroquinolin- 5-yl)-5,6,7,8-tetrahydropyrido[3,2-d]pyrimidin- 2-yl)-N-(3-(4-(2,6-dioxopiperidin-3-yl) benzofuran-2-yl)prop-2-yn-1-yl)picolinamide |
| B68 | 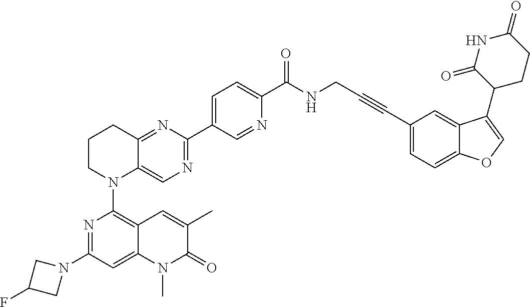 [SMILES: Cc1cc2c(N3CCCc4nc(-c5ccc(C(=O)NCC#Cc6ccc7occ(C8CCC(=O)NC8=O)c7c6)nc5)ncc43)nc(N3CC(F)C3)cc2n(C)c1=O] | N-(3-(3-(2,6-dioxopiperidin-3-yl)benzofuran- 5-yl)prop-2-yn-1-yl)-5-(5-(7-(3-fluoroazetidin- 1-yl)-1,3-dimethyl-2-oxo-1,2-dihydro-1,6- naphthyridin-5-yl)-5,6,7,8-tetrahydropyrido [3,2-d]pyrimidin-2-yl)picolinamide |
| B69 | 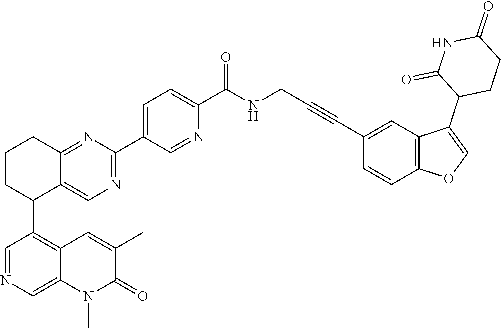 [SMILES: Cc1cc2c(C3CCCc4nc(-c5ccc(C(=O)NCC#Cc6ccc7occ(C8CCC(=O)NC8=O)c7c6)nc5)ncc43)cncc2n(C)c1=O] | 5-(5-(1,3-dimethyl-2-oxo-1,2-dihydro-1,7- naphthyridin-5-yl)-5,6,7,8-tetrahydropyrido [3,2-d]pyrimidin-2-yl)-N-(3-(3-(2,6-dioxo- piperidin-3-yl)benzofuran-5-yl)prop-2-yn-1- yl)picolinamide |
| B70 | 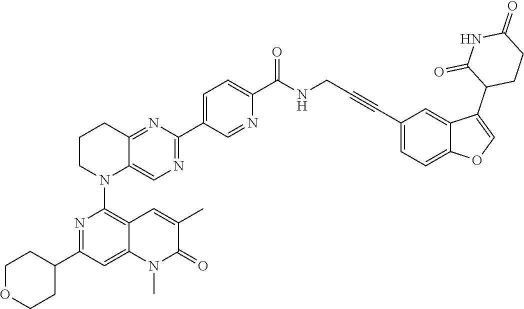 [SMILES: Cc1cc2c(N3CCCc4nc(-c5ccc(C(=O)NCC#Cc6ccc7occ(C8CCC(=O)NC8=O)c7c6)nc5)ncc43)nc(C3CCOCC3)cc2n(C)c1=O] | 5-(5-(1,3-dimethyl-2-oxo-7-(tetrahydro-2H- pyran-4-yl)-1,2-dihydro-1,6-naphthyridin- 5-yl)-5,6,7,8-tetrahydropyrido[3,2-d]pyrimidin- 2-yl)-N-(3-(3-(2,6-dioxopiperidin-3-yl) benzofuran-5-yl)prop-2-yn-1-yl)picolinamide |
| B71 | 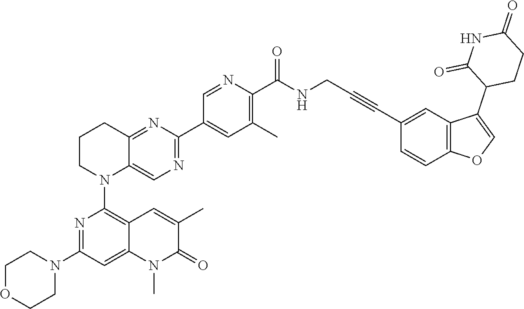 [SMILES: Cc1cc(-c2ncc3c(n2)CCCN3c2nc(N3CCOCC3)cc3c2cc(C)c(=O)n3C)cnc1C(=O)NCC#Cc1ccc2occ(C3CCC(=O)NC3=O)c2c1] | 5-(5-(1,3-dimethyl-7-morpholino-2-oxo-1,2- dihydro-1,6-naphthyridin-5-yl)-5,6,7,8- tetrahydropyrido[3,2-d]pyrimidin-2-yl)-N-(3- (3-(2,6-dioxopiperidin-3-yl)benzofuran-5-yl) prop-2-yn-1-yl)-3-methylpicolinamide |
| B72 | 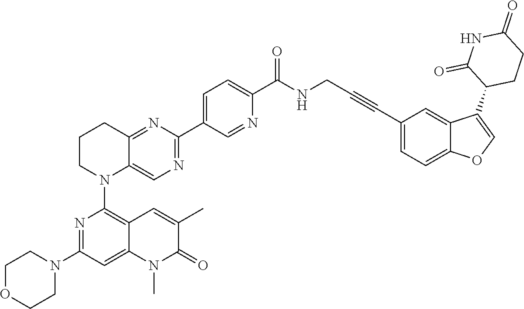 [SMILES: Cc1cc2c(N3CCCc4nc(-c5ccc(C(=O)NCC#Cc6ccc7occ([C@H]8CCC(=O)NC8=O)c7c6)nc5)ncc43)nc(N3CCOCC3)cc2n(C)c1=O] | (R)-5-(5-(1,3-dimethyl-7-morpholino-2-oxo- 1,2-dihydro-1,6-naphthyridin-5-yl)-5,6,7,8- tetrahydropyrido[3,2-d]pyrimidin-2-yl)-N-(3- (3-(2,6-dioxopiperidin-3-yl)benzofuran-5-yl) prop-2-yn-1-yl)picolinamide |
| B73 | 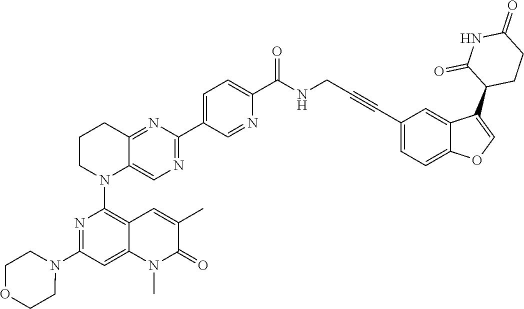 [SMILES: Cc1cc2c(N3CCCc4nc(-c5ccc(C(=O)NCC#Cc6ccc7occ([C@@H]8CCC(=O)NC8=O)c7c6)nc5)ncc43)nc(N3CCOCC3)cc2n(C)c1=O] | (S)-5-(5-(1,3-dimethyl-7-morpholino-2-oxo- 1,2-dihydro-1,6-naphthyridin-5-yl)-5,6,7,8- tetrahydropyrido[3,2-d]pyrimidin-2-yl)-N- (3-(3-(2,6-dioxopiperidin-3-yl)benzofuran- 5-yl)prop-2-yn-1-yl)picolinamide |
| B74 | 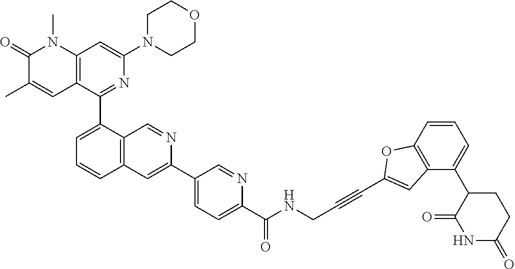 [SMILES: Cc1cc2c(-c3cccc4cc(-c5ccc(C(=O)NCC#Cc6cc7c(C8CCC(=O)NC8=O)cccc7o6)nc5)ncc34)nc(N3CCOCC3)cc2n(C)c1=O] | 5-(8-(1,3-dimethyl-7-morpholino-2-oxo-1,2- dihydro-1,6-naphthyridin-5-yl)isoquinolin- 3-yl)-N-(3-(4-(2,6-dioxopiperidin-3-yl) benzofuran-2-yl)prop-2-yn-1-yl)picolinamide |
| B76 | 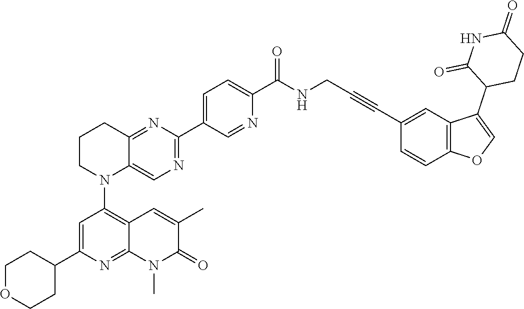 [SMILES: Cc1cc2c(N3CCCc4nc(-c5ccc(C(=O)NCC#Cc6ccc7occ(C8CCC(=O)NC8=O)c7c6)nc5)ncc43)cc(C3CCOCC3)nc2n(C)c1=O] | 5-(5-(6,8-dimethyl-7-oxo-2-(tetrahydro-2H- pyran-4-yl)-7,8-dihydro-1,8-naphthyridin-4-yl)- 5,6,7,8-tetrahydropyrido[3,2-d]pyrimidin-2-yl)- N-(3-(3-(2,6-dioxopiperidin-3-yl)benzofuran- 5-yl)prop-2-yn-1-yl)picolinamide |
| B77 | 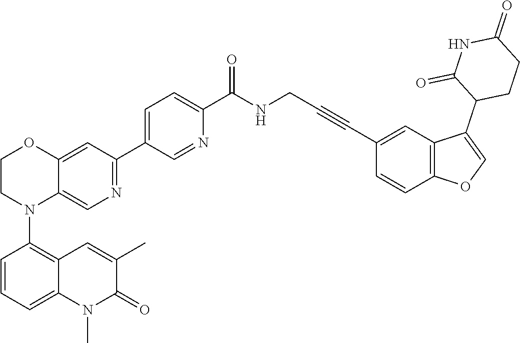 [SMILES: Cc1cc2c(N3CCOc4cc(-c5ccc(C(=O)NCC#Cc6ccc7occ(C8CCC(=O)NC8=O)c7c6)nc5)ncc43)cccc2n(C)c1=O] | 5-(4-(1,3-dimethyl-2-oxo-1,2-dihydroquinolin- 5-yl)-3,4-dihydro-2H-pyrido[4,3-b][1,4]oxazin- 7-yl)-N-(3-(3-(2,6-dioxopiperidin-3-yl) benzofuran-5-yl)prop-2-yn-1-yl)picolinamide |
| B78 | 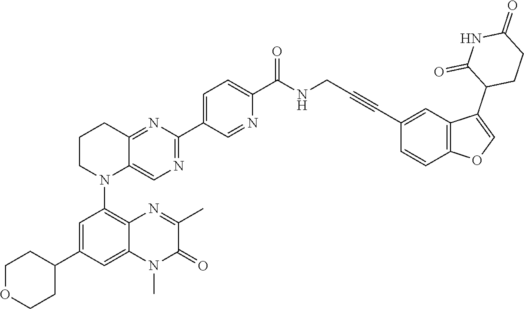 [SMILES: Cc1nc2c(N3CCCc4nc(-c5ccc(C(=O)NCC#Cc6ccc7occ(C8CCC(=O)NC8=O)c7c6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinoxalin-5-yl)- 5,6,7,8-tetrahydropyrido[3,2-d]pyrimidin-2-yl)- N-(3-(3-(2,6-dioxopiperidin-3-yl)benzofuran- 5-yl)prop-2-yn-1-yl)picolinamide |
| B79 | 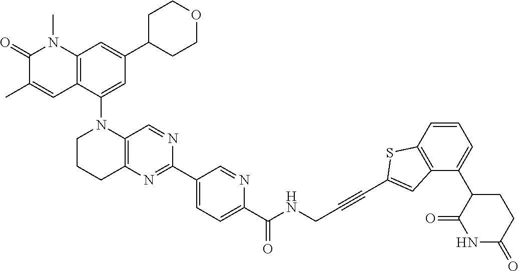 [SMILES: Cc1cc2c(N3CCCc4nc(-c5ccc(C(=O)NCC#Cc6cc7c(C8CCC(=O)NC8=O)cccc7s6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N- (3-(4-(2,6-dioxopiperidin-3-yl)benzo[b] thiophen-2-yl)prop-2-yn-1-yl)picolinamide |
| B80 | 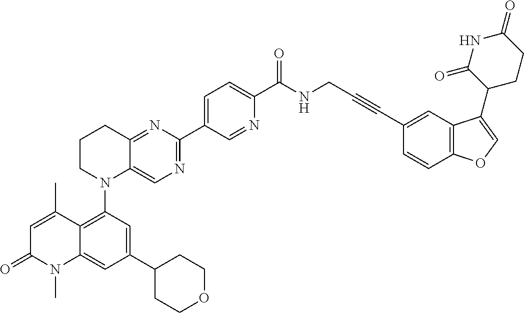 [SMILES: Cc1cc(=O)n(C)c2cc(C3CCOCC3)cc(N3CCCc4nc(-c5ccc(C(=O)NCC#Cc6ccc7occ(C8CCC(=O)NC8=O)c7c6)nc5)ncc43)c12] | 5-(5-(1,4-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (3-(2,6-dioxopiperidin-3-yl)benzofuran-5-yl) prop-2-yn-1-yl)picolinamide |
| B81 | 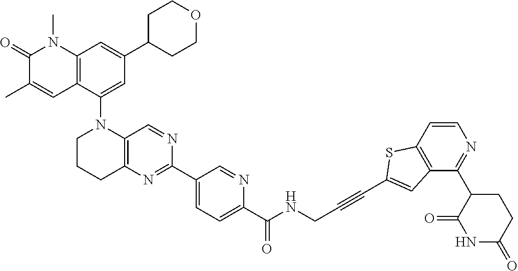 [SMILES: Cc1cc2c(N3CCCc4nc(-c5ccc(C(=O)NCC#Cc6cc7c(C8CCC(=O)NC8=O)nccc7s6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,6-dioxopiperidin-3-yl)thieno[3,2-c] pyridin-2-yl)prop-2-yn-1-yl)picolinamide |
| B82 | 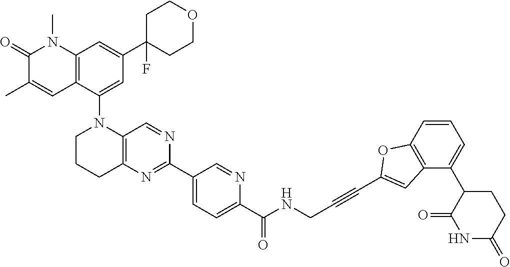 [SMILES: Cc1cc2c(N3CCCc4nc(-c5ccc(C(=O)NCC#Cc6cc7c(C8CCC(=O)NC8=O)cccc7o6)nc5)ncc43)cc(C3(F)CCOCC3)cc2n(C)c1=O] | N-(3-(4-(2,6-dioxopiperidin-3-yl)benzofuran-2- yl)prop-2-yn-1-yl)-5-(5-(7-(4-fluorotetrahydro- 2H-pyran-4-yl)-1,3-dimethyl-2-oxo-1,2- dihydroquinolin-5-yl)-5,6,7,8-tetrahydropyrido [3,2-d]pyrimidin-2-yl)picolinamide |
| B83 | 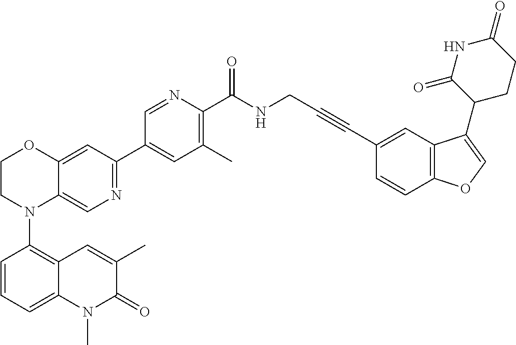 [SMILES: Cc1cc(-c2cc3c(cn2)N(c2cccc4c2cc(C)c(=O)n4C)CCO3)cnc1C(=O)NCC#Cc1ccc2occ(C3CCC(=O)NC3=O)c2c1] | 5-(4-(1,3-dimethyl-2-oxo-1,2-dihydroquinolin- 5-yl)-3,4-dihydro-2H-pyrido[4,3-b][1,4] oxazin-7-yl)-N-(3-(3-(2,6-dioxopiperidin- 3-yl)benzofuran-5-yl)prop-2-yn-1-yl)-3- methylpicolinamide |
| B84 | 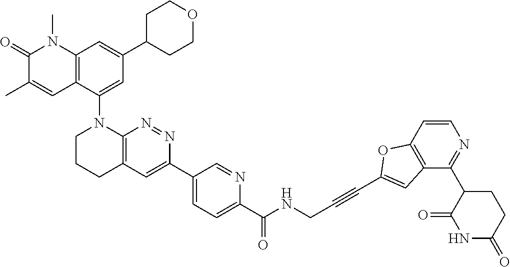 [SMILES: Cc1cc2c(N3CCCc4cc(-c5ccc(C(=O)NCC#Cc6cc7c(C8CCC(=O)NC8=O)nccc7o6)nc5)nnc43)cc(C3CCOCC3)cc2n(C)c1=O] | 5-(8-(1,3-dimethyl-2-oxo-7-(tetrahydro-2H- pyran-4-yl)-1,2-dihydroquinolin-5-yl)- 5,6,7,8-tetrahydropyrido[2,3-c]pyridazin-3- yl)-N-(3-(4-(2,6-dioxopiperidin-3-yl)furo [3,2-c]pyridin-2-yl)prop-2-yn-1-yl) picolinamide |
| B85 | 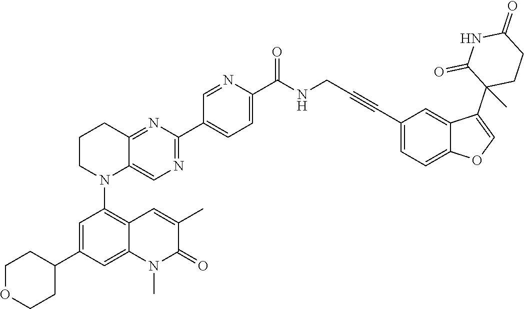 [SMILES: Cc1cc2c(N3CCCc4nc(-c5ccc(C(=O)NCC#Cc6ccc7occ(C8(C)CCC(=O)NC8=O)c7c6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N- (3-(3-(3-methyl-2,6-dioxopiperidin-3-yl) benzofuran-5-yl)prop-2-yn-1-yl)picolinamide |
| B86 | 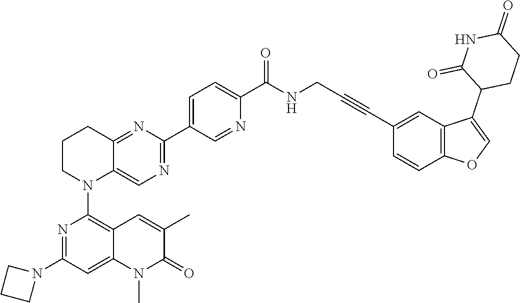 [SMILES: Cc1cc2c(N3CCCc4nc(-c5ccc(C(=O)NCC#Cc6ccc7occ(C8CCC(=O)NC8=O)c7c6)nc5)ncc43)nc(N3CCC3)cc2n(C)c1=O] | 5-(5-(7-(azetidin-1-yl)-1,3-dimethyl-2-oxo-1,2- dihydro-1,6-naphthyridin-5-yl)-5,6,7,8- tetrahydropyrido[3,2-d]pyrimidin-2-yl)-N-(3- (3-(2,6-dioxopiperidin-3-yl)benzofuran-5-yl) prop-2-yn-1-yl)picolinamide |
| B87 | 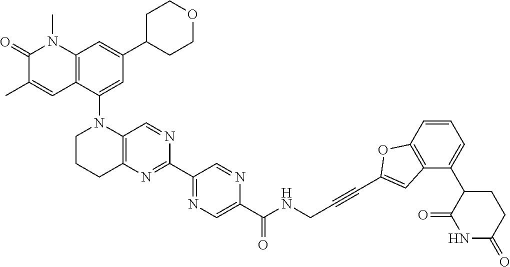 [SMILES: Cc1cc2c(N3CCCc4nc(-c5cnc(C(=O)NCC#Cc6cc7c(C8CCC(=O)NC8=O)cccc7o6)cn5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,6-dioxopiperidin-3-yl)benzofuran-2-yl) prop-2-yn-1-yl)pyrazine-2-carboxamide |
| B88 | 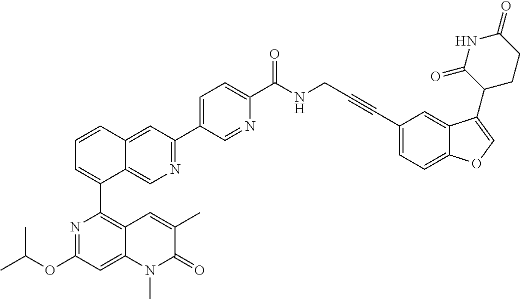 [SMILES: Cc1cc2c(-c3cccc4cc(-c5ccc(C(=O)NCC#Cc6ccc7occ(C8CCC(=O)NC8=O)c7c6)nc5)ncc34)nc(OC(C)C)cc2n(C)c1=O] | N-(3-(3-(2,6-dioxopiperidin-3-yl)benzofuran- 5-yl)prop-2-yn-1-yl)-5-(8-(7-isopropoxy-1,3- dimethyl-2-oxo-1,2-dihydro-1,6-naphthyridin- 5-yl)isoquinolin-3-yl)picolinamide |
| B89 | 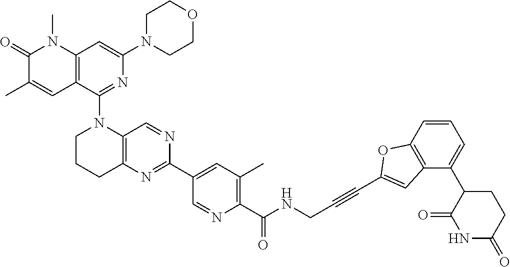 [SMILES: Cc1cc(-c2ncc3c(n2)CCCN3c2nc(N3CCOCC3)cc3c2cc(C)c(=O)n3C)cnc1C(=O)NCC#Cc1cc2c(C3CCC(=O)NC3=O)cccc2o1] | 5-(5-(1,3-dimethyl-7-morpholino-2-oxo-1,2- dihydro-1,6-naphthyridin-5-yl)-5,6,7,8- tetrahydropyrido[3,2-d]pyrimidin-2-yl)-N-(3- (4-(2,6-dioxopiperidin-3-yl)benzofuran-2-yl) prop-2-yn-1-yl)-3-methylpicolinamide |
| B90 | 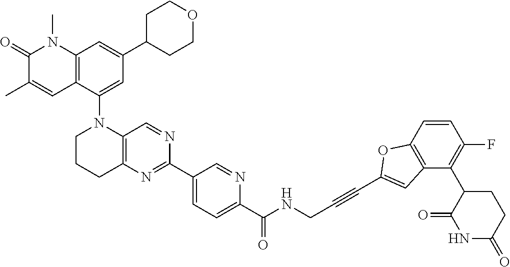 [SMILES: Cc1cc2c(N3CCCc4nc(-c5ccc(C(=O)NCC#Cc6cc7c(C8CCC(=O)NC8=O)c(F)ccc7o6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,6-dioxopiperidin-3-yl)-5-fluorobenzo- furan-2-yl)prop-2-yn-1-yl)picolinamide |
| B91 | 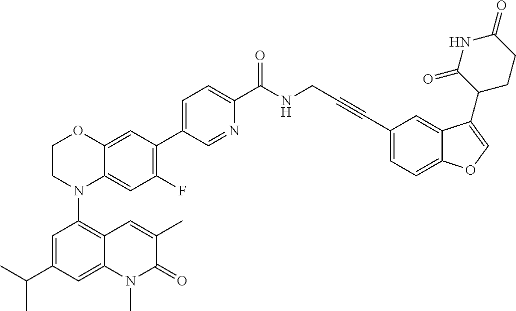 [SMILES: Cc1cc2c(N3CCOc4cc(-c5ccc(C(=O)NCC#Cc6ccc7occ(C8CCC(=O)NC8=O)c7c6)nc5)c(F)cc43)cc(C(C)C)cc2n(C)c1=O] | N-(3-(3-(2,6-dioxopiperidin-3-yl)benzofuran- 5-yl)prop-2-yn-1-yl)-5-(6-fluoro-4-(7- isopropyl-1,3-dimethyl-2-oxo-1,2- dihydroquinolin-5-yl)-3,4-dihydro-2H- benzo[b][1,4]oxazin-7-yl)picolinamide |
| B92 | 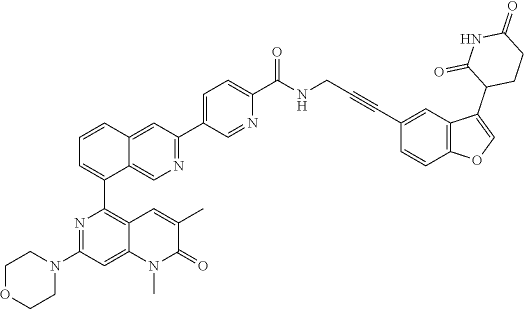 [SMILES: Cc1cc2c(-c3cccc4cc(-c5ccc(C(=O)NCC#Cc6ccc7occ(C8CCC(=O)NC8=O)c7c6)nc5)ncc34)nc(N3CCOCC3)cc2n(C)c1=O] | 5-(8-(1,3-dimethyl-7-morpholino-2-oxo-1,2- dihydro-1,6-naphthyridin-5-yl)isoquinolin-3- yl)-N-(3-(3-(2,6-dioxopiperidin-3-yl) benzofuran-5-yl)prop-2-yn-1-yl)picolinamide |
| B93 | 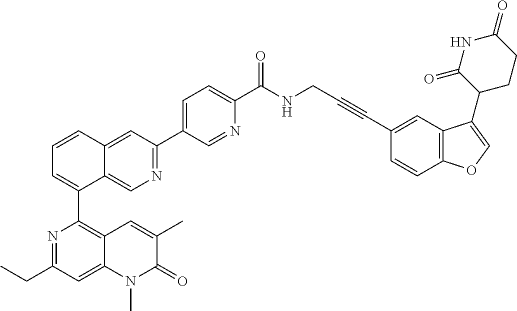 [SMILES: CCc1cc2c(cc(C)c(=O)n2C)c(-c2cccc3cc(-c4ccc(C(=O)NCC#Cc5ccc6occ(C7CCC(=O)NC7=O)c6c5)nc4)ncc23)n1] | N-(3-(3-(2,6-dioxopiperidin-3-yl)benzofuran- 5-yl)prop-2-yn-1-yl)-5-(8-(7-ethyl-1,3- dimethyl-2-oxo-1,2-dihydro-1,6-naphthyridin- 5-yl)isoquinolin-3-yl)picolinamide |
| B94 | 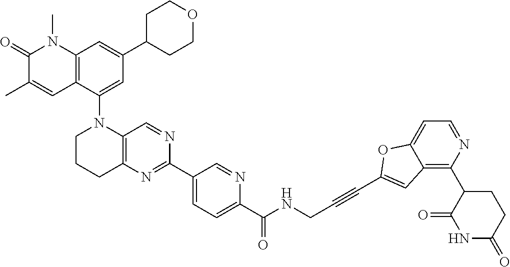 [SMILES: Cc1cc2c(N3CCCc4nc(-c5ccc(C(=O)NCC#Cc6cc7c(C8CCC(=O)NC8=O)nccc7o6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,6-dioxopiperidin-3-yl)furo[3,2-c]pyridin- 2-yl)prop-2-yn-1-yl)picolinamide |
| B95 | 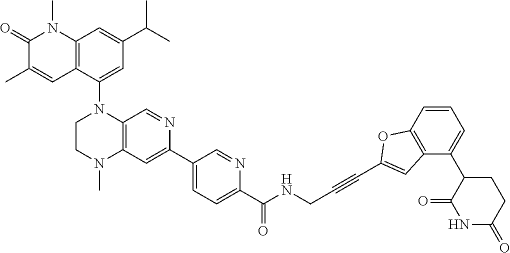 [SMILES: Cc1cc2c(N3CCN(C)c4cc(-c5ccc(C(=O)NCC#Cc6cc7c(C8CCC(=O)NC8=O)cccc7o6)nc5)ncc43)cc(C(C)C)cc2n(C)c1=O] | N-(3-(4-(2,6-dioxopiperidin-3-yl)benzofuran- 2-yl)prop-2-yn-1-yl)-5-(4-(7-isopropyl-1,3- dimethyl-2-oxo-1,2-dihydroquinolin-5-yl)-1- methyl-1,2,3,4-tetrahydropyrido[3,4-b]pyrazin- 7-yl)picolinamide |
| B96 | 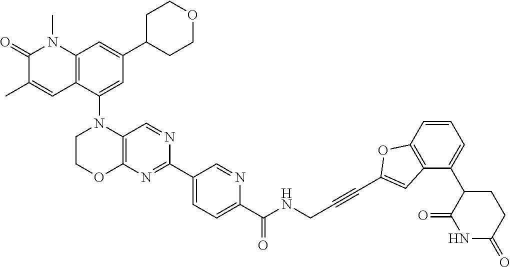 [SMILES: Cc1cc2c(N3CCOc4nc(-c5ccc(C(=O)NCC#Cc6cc7c(C8CCC(=O)NC8=O)cccc7o6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-6,7- dihydro-5H-pyrimido[4,5-b][1,4]oxazin-2- yl)-N-(3-(4-(2,6-dioxopiperidin-3-yl) benzofuran-2-yl)prop-2-yn-1-yl)picolinamide |
| B97 | 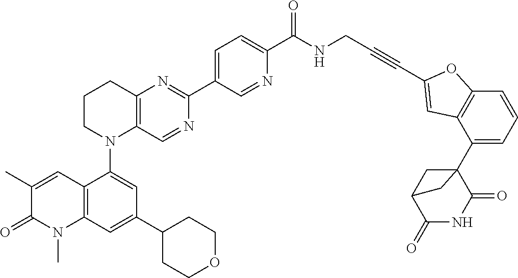 [SMILES: Cc1cc2c(N3CCCc4nc(-c5ccc(C(=O)NCC#Cc6cc7c(C89CC(C8)C(=O)NC9=O)cccc7o6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,4-dioxo-3-azabicyclo[3.1.1]heptan-1-yl) benzofuran-2-yl)prop-2-yn-1-yl)picolinamide |
| B98 | 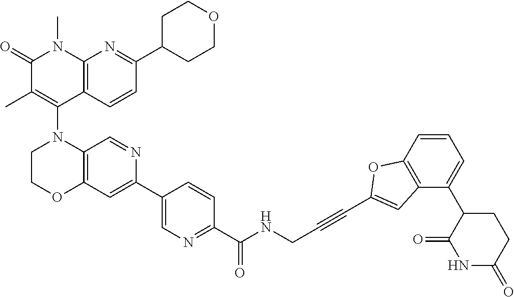 [SMILES: Cc1c(N2CCOc3cc(-c4ccc(C(=O)NCC#Cc5cc6c(C7CCC(=O)NC7=O)cccc6o5)nc4)ncc32)c2ccc(C3CCOCC3)nc2n(C)c1=O] | 5-(4-(6,8-dimethyl-7-oxo-2-(tetrahydro-2H- pyran-4-yl)-7,8-dihydro-1,8-naphthyridin-4- yl)-3,4-dihydro-2H-pyrido[4,3-b][1,4]oxazin- 7-yl)-N-(3-(4-(2,6-dioxopiperidin-3-yl) benzofuran-2-yl)prop-2-yn-1-yl)picolinamide |
| B99 | 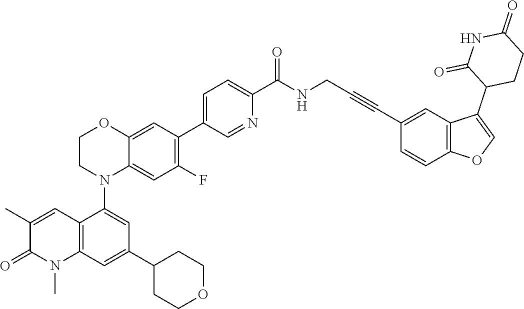 [SMILES: Cc1cc2c(N3CCOc4cc(-c5ccc(C(=O)NCC#Cc6ccc7occ(C8CCC(=O)NC8=O)c7c6)nc5)c(F)cc43)cc(C3CCOCC3)cc2n(C)c1=O] | 5-(4-(1,3-dimethyl-2-oxo-7-(tetrahydro-2H- pyran-4-yl)-1,2-dihydroquinolin-5-yl)-6-fluoro- 3,4-dihydro-2H-benzo[b][1,4]oxazin-7-yl)-N- (3-(3-(2,6-dioxopiperidin-3-yl)benzofuran-5- yl)prop-2-yn-1-yl)picolinamide |
| B100 | 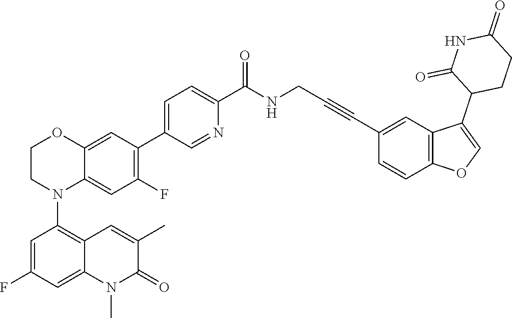 [SMILES: Cc1cc2c(N3CCOc4cc(-c5ccc(C(=O)NCC#Cc6ccc7occ(C8CCC(=O)NC8=O)c7c6)nc5)c(F)cc43)cc(F)cc2n(C)c1=O] | N-(3-(3-(2,6-dioxopiperidin-3-yl)benzofuran-5- yl)prop-2-yn-1-yl)-5-(6-fluoro-4-(7-fluoro-1,3- dimethyl-2-oxo-1,2-dihydroquinolin-5-yl)-3,4- dihydro-2H-benzo[b][1,4]oxazin-7-yl) picolinamide |
| B101 | 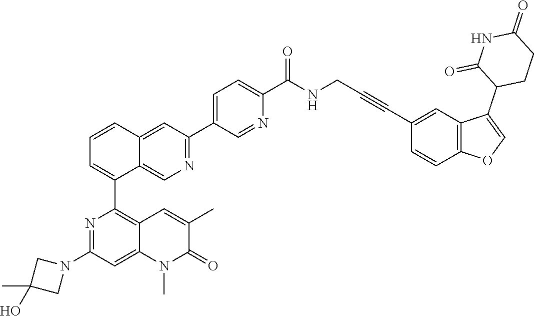 [SMILES: Cc1cc2c(-c3cccc4cc(-c5ccc(C(=O)NCC#Cc6ccc7occ(C8CCC(=O)NC8=O)c7c6)nc5)ncc34)nc(N3CC(C)(O)C3)cc2n(C)c1=O] | N-(3-(3-(2,6-dioxopiperidin-3-yl)benzofuran- 5-yl)prop-2-yn-1-yl)-5-(8-(7-(3-hydroxy-3- methylazetidin-1-yl)-1,3-dimethyl-2-oxo- 1,2-dihydro-1,6-naphthyridin-5-yl) isoquinolin-3-yl)picolinamide |
| B102 | 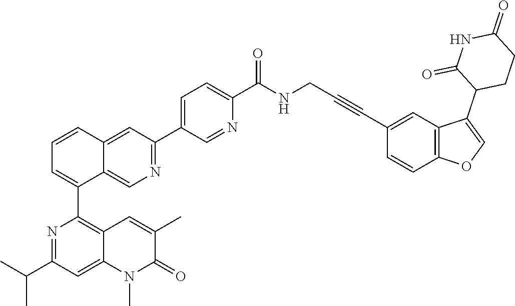 [SMILES: Cc1cc2c(-c3cccc4cc(-c5ccc(C(=O)NCC#Cc6ccc7occ(C8CCC(=O)NC8=O)c7c6)nc5)ncc34)nc(C(C)C)cc2n(C)c1=O] | N-(3-(3-(2,6-dioxopiperidin-3-yl)benzofuran-5- yl)prop-2-yn-1-yl)-5-(8-(7-isopropyl-1,3- dimethyl-2-oxo-1,2-dihydro-1,6-naphthyridin- 5-yl)isoquinolin-3-yl)picolinamide |
| B103 | 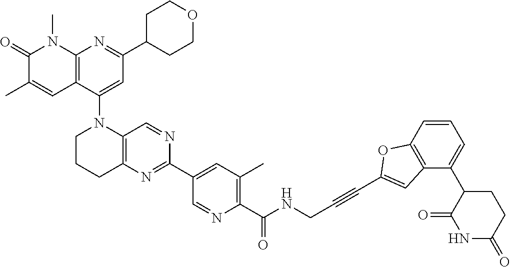 [SMILES: Cc1cc(-c2ncc3c(n2)CCCN3c2cc(C3CCOCC3)nc3c2cc(C)c(=O)n3C)cnc1C(=O)NCC#Cc1cc2c(C3CCC(=O)NC3=O)cccc2o1] | 5-(5-(6,8-dimethyl-7-oxo-2-(tetrahydro-2H- pyran-4-yl)-7,8-dihydro-1,8-naphthyridin-4- yl)-5,6,7,8-tetrahydropyrido[3,2-d]pyrimidin- 2-yl)-N-(3-(4-(2,6-dioxopiperidin-3-yl) benzofuran-2-yl)prop-2-yn-1-yl)-3- methylpicolinamide |
| B104 | 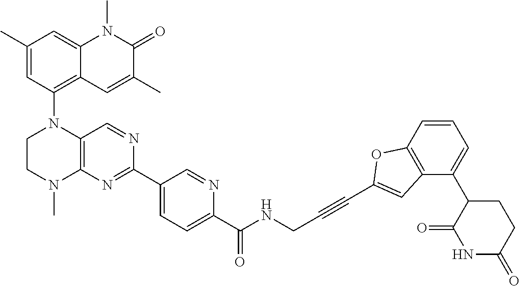 [SMILES: Cc1cc(N2CCN(C)c3nc(-c4ccc(C(=O)NCC#Cc5cc6c(C7CCC(=O)NC7=O)cccc6o5)nc4)ncc32)c2cc(C)c(=O)n(C)c2c1] | N-(3-(4-(2,6-dioxopiperidin-3-yl)benzofuran-2- yl)prop-2-yn-1-yl)-5-(8-methyl-5-(1,3,7- trimethyl-2-oxo-1,2-dihydroquinolin-5-yl)- 5,6,7,8-tetrahydropteridin-2-yl)picolinamide |
| B105 | 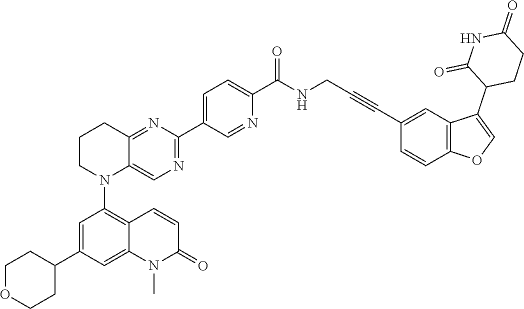 [SMILES: Cn1c(=O)ccc2c(N3CCCc4nc(-c5ccc(C(=O)NCC#Cc6ccc7occ(C8CCC(=O)NC8=O)c7c6)nc5)ncc43)cc(C3CCOCC3)cc21] | N-(3-(3-(2,6-dioxopiperidin-3-yl)benzofuran-5- yl)prop-2-yn-1-yl)-5-(5-(1-methyl-2-oxo-7- (tetrahydro-2H-pyran-4-yl)-1,2-dihydro- quinolin-5-yl)-5,6,7,8-tetrahydropyrido[3,2-d] pyrimidin-2-yl)picolinamide |
| B106 | 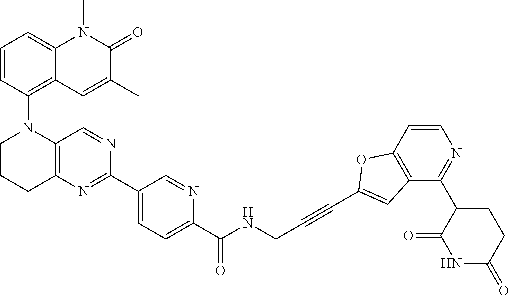 [SMILES: Cc1cc2c(N3CCCc4nc(-c5ccc(C(=O)NCC#Cc6cc7c(C8CCC(=O)NC8=O)nccc7o6)nc5)ncc43)cccc2n(C)c1=O] | 5-(5-(1,3-dimethyl-2-oxo-1,2-dihydroquinolin- 5-yl)-5,6,7,8-tetrahydropyrido[3,2-d]pyrimidin- 2-yl)-N-(3-(4-(2,6-dioxopiperidin-3-yl)furo [3,2-c]pyridin-2-yl)prop-2-yn-1-yl) picolinamide |
| B107 | 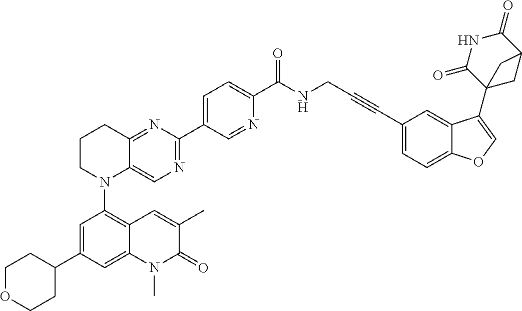 [SMILES: Cc1cc2c(N3CCCc4nc(-c5ccc(C(=O)NCC#Cc6ccc7occ(C89CC(C8)C(=O)NC9=O)c7c6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (3-(2,4-dioxo-3-azabicyclo[3.1.1]heptan-1- yl)benzofuran-5-yl)prop-2-yn-1-yl) picolinamide |
| B108 | 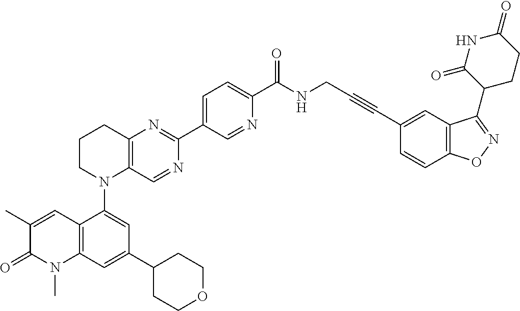 [SMILES: Cc1cc2c(N3CCCc4nc(-c5ccc(C(=O)NCC#Cc6ccc7onc(C8CCC(=O)NC8=O)c7c6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (3-(2,6-dioxopiperidin-3-yl)benzo[d]isoxazol- 5-yl)prop-2-yn-1-yl)picolinamide |
| B109 | 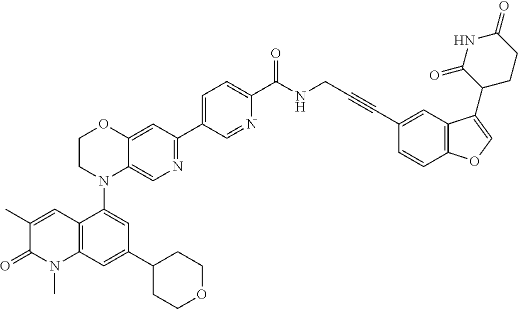 [SMILES: Cc1cc2c(N3CCOc4cc(-c5ccc(C(=O)NCC#Cc6ccc7occ(C8CCC(=O)NC8=O)c7c6)nc5)ncc43)cc(C3CCOCC3)cc2n(C)c1=O] | 5-(4-(1,3-dimethyl-2-oxo-7-(tetrahydro-2H- pyran-4-yl)-1,2-dihydroquinolin-5-yl)-3,4- dihydro-2H-pyrido[4,3-b][1,4]oxazin-7-yl)-N- (3-(3-(2,6-dioxopiperidin-3-yl)benzofuran-5- yl)prop-2-yn-1-yl)picolinamide |
| B110 | 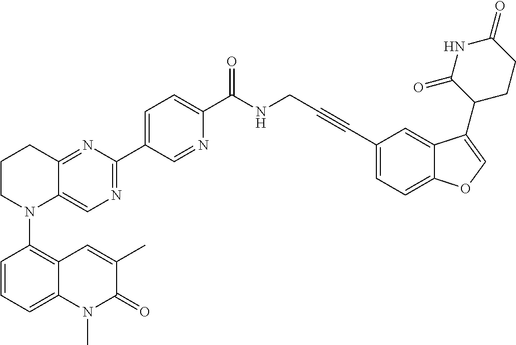 [SMILES: Cc1cc2c(N3CCCc4nc(-c5ccc(C(=O)NCC#Cc6ccc7occ(C8CCC(=O)NC8=O)c7c6)nc5)ncc43)cccc2n(C)c1=O] | 5-(5-(1,3-dimethyl-2-oxo-1,2-dihydroquinolin- 5-yl)-5,6,7,8-tetrahydropyrido[3,2-d]pyrimidin- 2-yl)-N-(3-(3-(2,6-dioxopiperidin-3-yl) benzofuran-5-yl)prop-2-yn-1-yl)picolinamide |
| B111 | 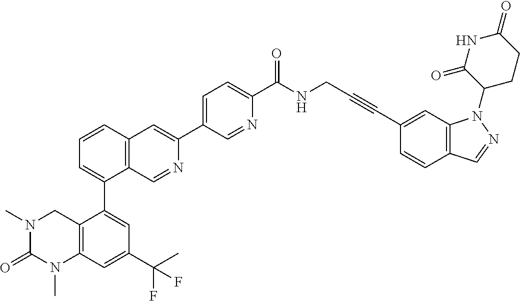 [SMILES: CN1Cc2c(-c3cccc4cc(-c5ccc(C(=O)NCC#Cc6ccc7cnn(C8CCC(=O)NC8=O)c7c6)nc5)ncc34)cc(C(C)(F)F)cc2N(C)C1=O] | 5-(8-(7-(1,1-difluoroethyl)-1,3-dimethyl-2-oxo- 1,2,3,4-tetrahydroquinazolin-5-yl)isoquinolin- 3-yl)-N-(3-(1-(2,6-dioxopiperidin-3-yl)-1H- indazol-6-yl)prop-2-yn-1-yl)picolinamide |
| B112 | 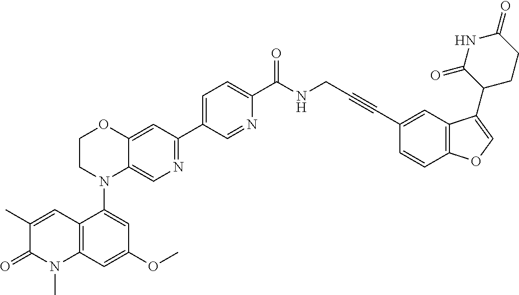 [SMILES: COc1cc(N2CCOc3cc(-c4ccc(C(=O)NCC#Cc5ccc6occ(C7CCC(=O)NC7=O)c6c5)nc4)ncc32)c2cc(C)c(=O)n(C)c2c1] | N-(3-(3-(2,6-dioxopiperidin-3-yl)benzofuran- 5-yl)prop-2-yn-1-yl)-5-(4-(7-methoxy-1,3- dimethyl-2-oxo-1,2-dihydroquinolin-5-yl)-3,4- dihydro-2H-pyrido[4,3-b][1,4]oxazin-7-yl) picolinamide |
| B113 | 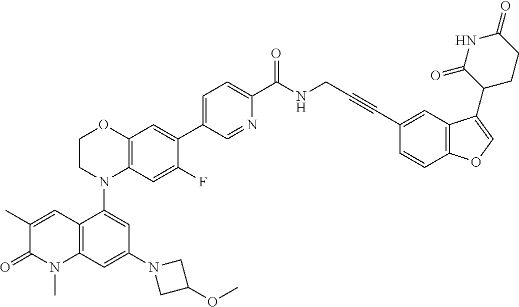 [SMILES: COC1CN(c2cc(N3CCOc4cc(-c5ccc(C(=O)NCC#Cc6ccc7occ(C8CCC(=O)NC8=O)c7c6)nc5)c(F)cc43)c3cc(C)c(=O)n(C)c3c2)C1] | N-(3-(3-(2,6-dioxopiperidin-3-yl)benzofuran-5- yl)prop-2-yn-1-yl)-5-(6-fluoro-4-(7-(3- methoxyazetidin-1-yl)-1,3-dimethyl-2-oxo-1,2- dihydroquinolin-5-yl)-3,4-dihydro-2H- benzo[b][1,4]oxazin-7-yl)picolinamide |
| B114 | 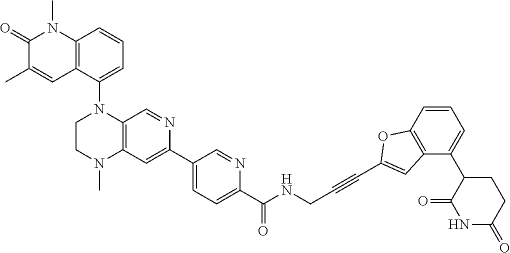 [SMILES: Cc1cc2c(N3CCN(C)c4cc(-c5ccc(C(=O)NCC#Cc6cc7c(C8CCC(=O)NC8=O)cccc7o6)nc5)ncc43)cccc2n(C)c1=O] | 5-(4-(1,3-dimethyl-2-oxo-1,2-dihydroquinolin- 5-yl)-1-methyl-1,2,3,4-tetrahydropyrido [3,4-b]pyrazin-7-yl)-N-(3-(4-(2,6-dioxo- piperidin-3-yl)benzofuran-2-yl)prop-2-yn-1- yl)picolinamide |
| B115 | 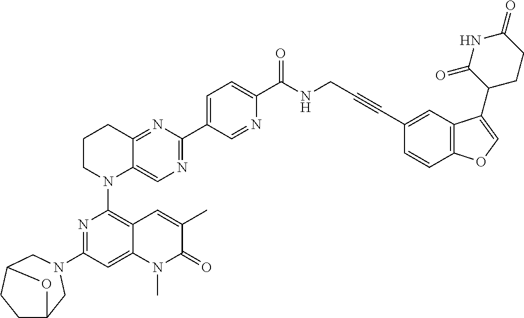 [SMILES: Cc1cc2c(N3CCCc4nc(-c5ccc(C(=O)NCC#Cc6ccc7occ(C8CCC(=O)NC8=O)c7c6)nc5)ncc43)nc(N3CC4CCC(C3)O4)cc2n(C)c1=O] | 5-(5-(7-(8-oxa-3-azabicyclo[3.2.1]octan-3-yl)- 1,3-dimethyl-2-oxo-1,2-dihydro-1,6- naphthyridin-5-yl)-5,6,7,8-tetrahydropyrido [3,2-d]pyrimidin-2-yl)-N-(3-(3-(2,6- dioxopiperidin-3-yl)benzofuran-5-yl)prop-2- yn-1-yl)picolinamide |
| B116 | 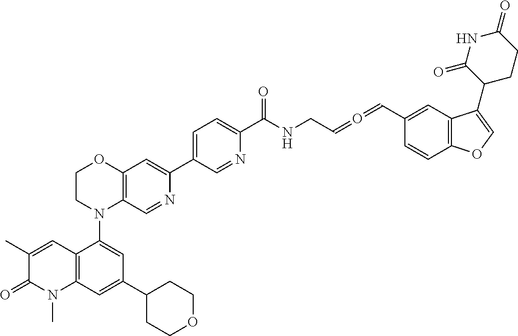 | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(4- (3-(2,6-dioxopiperidin-3-yl)benzofuran-5-yl) buta-2,3-dien-1-yl)picolinamide |
| B117 | 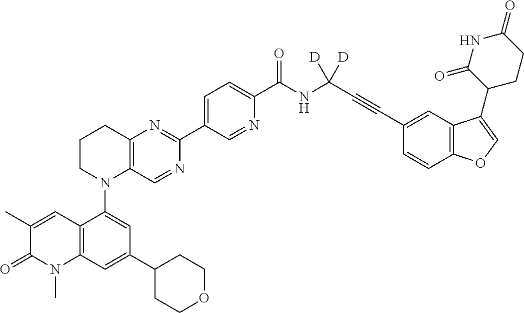 [SMILES: [2H]C([2H])(C#Cc1ccc2occ(C3CCC(=O)NC3=O)c2c1)NC(=O)c1ccc(-c2ncc3c(n2)CCCN3c2cc(C3CCOCC3)cc3c2cc(C)c(=O)n3C)cn1] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (3-(2,6-dioxopiperidin-3-yl)benzofuran-5-yl) prop-2-yn-1-yl-1,1-d2)picolinamide |
| B118 | 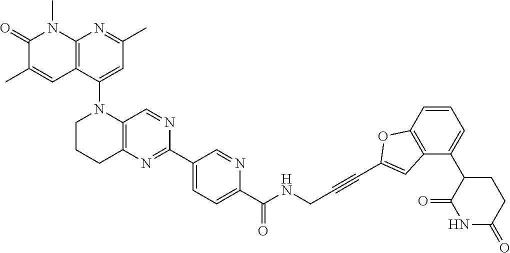 [SMILES: Cc1cc(N2CCCc3nc(-c4ccc(C(=O)NCC#Cc5cc6c(C7CCC(=O)NC7=O)cccc6o5)nc4)ncc32)c2cc(C)c(=O)n(C)c2n1] | N-(3-(4-(2,6-dioxopiperidin-3-yl)benzofuran-2- yl)prop-2-yn-1-yl)-5-(5-(2,6,8-trimethyl-7-oxo- 7,8-dihydro-1,8-naphthyridin-4-yl)-5,6,7,8- tetrahydropyrido[3,2-d]pyrimidin-2-yl) picolinamide |
| B119 | 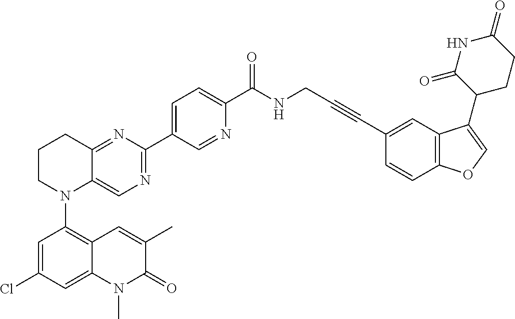 [SMILES: Cc1cc2c(N3CCCc4nc(-c5ccc(C(=O)NCC#Cc6ccc7occ(C8CCC(=O)NC8=O)c7c6)nc5)ncc43)cc(Cl)cc2n(C)c1=O] | 5-(5-(7-chloro-1,3-dimethyl-2-oxo-1,2- dihydroquinolin-5-yl)-5,6,7,8-tetrahydro[3,2- d]pyrimidin-2-yl)-N-(3-(3-(2,6-dioxopiperidin- 3-yl)benzofuran-5-yl)prop-2-yn-1-yl) picolinamide |
| B120 | 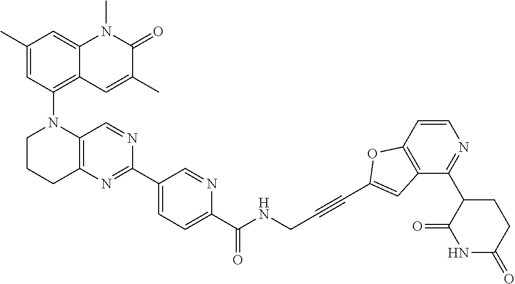 [SMILES: Cc1cc(N2CCCc3nc(-c4ccc(C(=O)NCC#Cc5cc6c(C7CCC(=O)NC7=O)nccc6o5)nc4)ncc32)c2cc(C)c(=O)n(C)c2c1] | N-(3-(4-(2,6-dioxopiperidin-3-yl)furo[3,2-c] pyridin-2-yl)prop-2-yn-1-yl)-5-(5-(1,3,7- trimethyl-2-oxo-1,2-dihydroquinolin-5-yl)- 5,6,7,8-tetrahydropyrido[3,2-d]pyrimidin-2-yl) picolinamide |
| B121 | 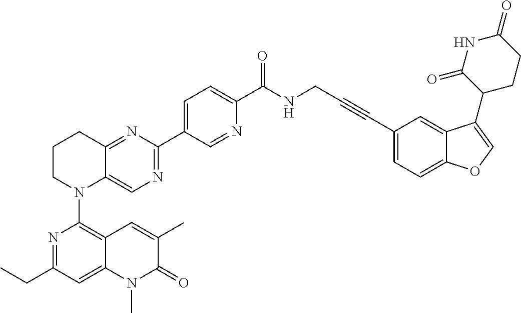 [SMILES: CCc1cc2c(cc(C)c(=O)n2C)c(N2CCCc3nc(-c4ccc(C(=O)NCC#Cc5ccc6occ(C7CCC(=O)NC7=O)c6c5)nc4)ncc32)n1] | N-(3-(3-(2,6-dioxopiperidin-3-yl)benzofuran-5- yl)prop-2-yn-1-yl)-5-(5-(7-ethyl-1,3-dimethyl- 2-oxo-1,2-dihydro-1,6-naphthyridin-5-yl)- 5,6,7,8-tetrahydropyrido[3,2-d]pyrimidin-2- yl)picolinamide |
| B122 | 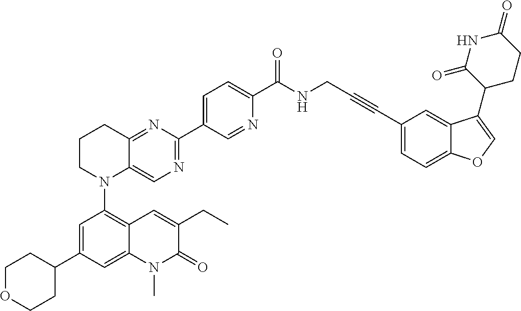 [SMILES: CCc1cc2c(N3CCCc4nc(-c5ccc(C(=O)NCC#Cc6ccc7occ(C8CCC(=O)NC8=O)c7c6)nc5)ncc43)cc(C3CCOCC3)cc2n(C)c1=O] | N-(3-(3-(2,6-dioxopiperidin-3-yl)benzofuran-5- yl)prop-2-yn-1-yl)-5-(5-(3-ethyl-1-methyl-2- oxo-7-(tetrahydro-2H-pyran-4-yl)-1,2- dihydroquinolin-5-yl)-5,6,7,8-tetrahydropyrido [3,2-d]pyrimidin-2-yl)picolinamide |
| B123 | 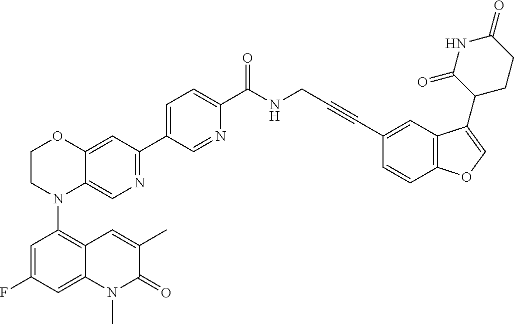 [SMILES: Cc1cc2c(N3CCOc4cc(-c5ccc(C(=O)NCC#Cc6ccc7occ(C8CCC(=O)NC8=O)c7c6)nc5)ncc43)cc(F)cc2n(C)c1=O] | N-(3-(3-(2,6-dioxopiperidin-3-yl)benzofuran-5- yl)prop-2-yn-1-yl)-5-(4-(7-fluoro-1,3-dimethyl- 2-oxo-1,2-dihydroquinolin-5-yl)-3,4-dihydro- 2H-pyrido[4,3-b][1,4]oxazin-7-yl)picolinamide |
| B124 | 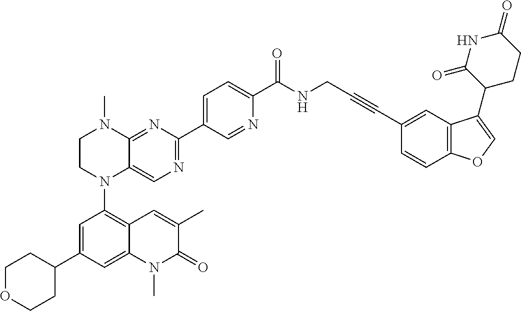 [SMILES: Cc1cc2c(N3CCN(C)c4nc(-c5ccc(C(=O)NCC#Cc6ccc7occ(C8CCC(=O)NC8=O)c7c6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-8- methyl-5,6,7,8-tetrahydropteridin-2-yl)-N-(3- (3-(2,6-dioxopiperidin-3-yl)benzofuran-5-yl) prop-2-yn-1-yl)picolinamide |
| B125 | 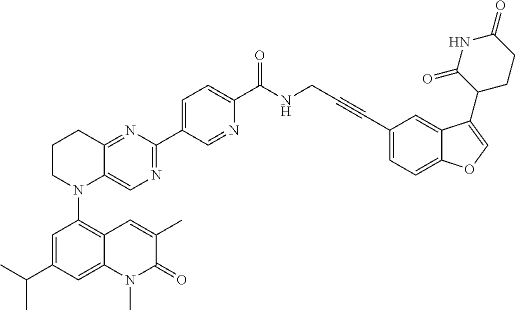 [SMILES: Cc1cc2c(N3CCCc4nc(-c5ccc(C(=O)NCC#Cc6ccc7occ(C8CCC(=O)NC8=O)c7c6)nc5)ncc43)cc(C(C)C)cc2n(C)c1=O] | N-(3-(3-(2,6-dioxopiperidin-3-yl)benzofuran-5- yl)prop-2-yn-1-yl)-5-(5-(7-isopropyl-1,3- dimethyl-2-oxo-1,2-dihydroquinolin-5-yl)- 5,6,7,8-tetrahydropyrido[3,2-d]pyrimidin-2- yl)picolinamide |
| B126 | 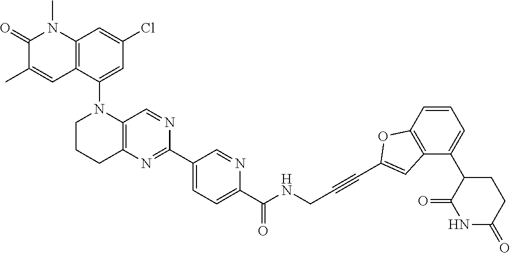 [SMILES: Cc1cc2c(N3CCCc4nc(-c5ccc(C(=O)NCC#Cc6cc7c(C8CCC(=O)NC8=O)cccc7o6)nc5)ncc43)cc(Cl)cc2n(C)c1=O] | 5-(5-(7-chloro-1,3-dimethyl-2-oxo-1,2- dihydroquinolin-5-yl)-5,6,7,8-tetrahydropyrido [3,2-d]pyrimidin-2-yl)-N-(3-(4-(2,6-dioxo- piperidin-3-yl)benzofuran-2-yl)prop-2-yn-1- yl)picolinamide |
| B127 | 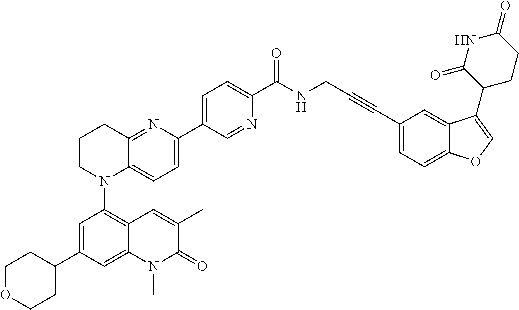 [SMILES: Cc1cc2c(N3CCCc4nc(-c5ccc(C(=O)NCC#Cc6ccc7occ(C8CCC(=O)NC8=O)c7c6)nc5)ccc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydro-1,5-naphthyridin-2-yl)-N-(3-(3- (2,6-dioxopiperidin-3-yl)benzofuran-5-yl) prop-2-yn-1-yl)picolinamide |
| B128 | 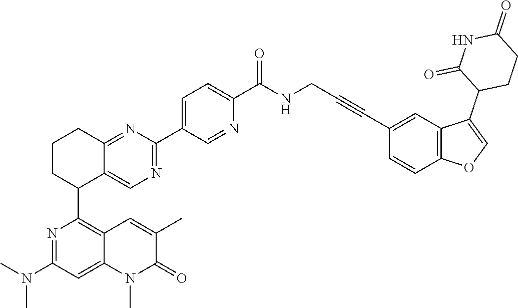 [SMILES: Cc1cc2c(C3CCCc4nc(-c5ccc(C(=O)NCC#Cc6ccc7occ(C8CCC(=O)NC8=O)c7c6)nc5)ncc43)nc(N(C)C)cc2n(C)c1=O] | 5-(5-(7-(dimethylamino)-1,3-dimethyl-2-oxo- 1,2-dihydro-1,6-naphthyridin-5-yl)-5,6,7,8- tetrahydropyrido[3,2-d]pyrimidin-2-yl)-N-(3- (3-(2,6-dioxopiperidin-3-yl)benzofuran-5-yl) prop-2-yn-1-yl)picolinamide |
| B129 | 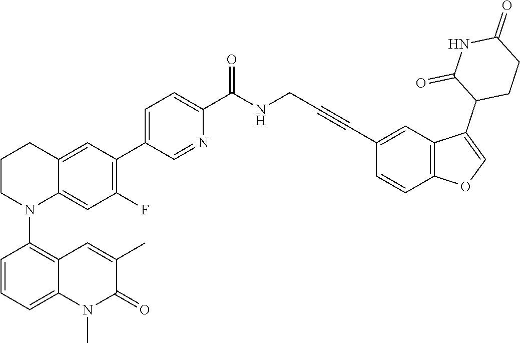 [SMILES: Cc1cc2c(N3CCCc4cc(-c5ccc(C(=O)NCC#Cc6ccc7occ(C8CCC(=O)NC8=O)c7c6)nc5)c(F)cc43)cccc2n(C)c1=O] | N-(3-(3-(2,6-dioxopiperidin-3-yl)benzofuran- 5-yl)prop-2-yn-1-yl)-5-(7-fluoro-1′,3′-dimethyl- 2′-oxo-1′,2′,3,4-tetrahydro-2H-[1,5′- biquinolin]-6-yl)picolinamide |
| B130 | 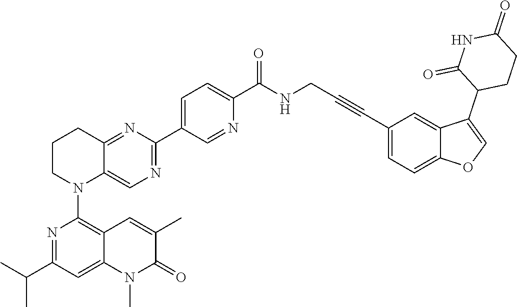 [SMILES: Cc1cc2c(N3CCCc4nc(-c5ccc(C(=O)NCC#Cc6ccc7occ(C8CCC(=O)NC8=O)c7c6)nc5)ncc43)nc(C(C)C)cc2n(C)c1=O] | N-(3-(3-(2,6-dioxopiperidin-3-yl)benzofuran- 5-yl)prop-2-yn-1-yl)-5-(5-(7-isopropyl-1,3- dimethyl-2-oxo-1,2-dihydro-1,6-naphthyridin- 5-yl)-5,6,7,8-tetrahydropyrido[3,2-d]pyrimidin- 2-yl)picolinamide |
| B131 | 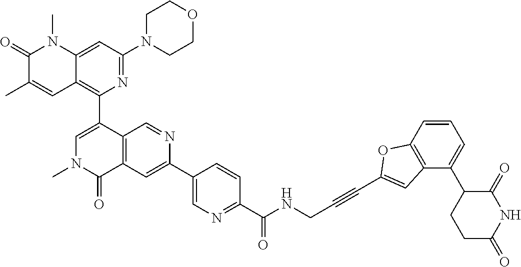 [SMILES: Cc1cc2c(-c3cn(C)c(=O)c4cc(-c5ccc(C(=O)NCC#Cc6cc7c(C8CCC(=O)NC8=O)cccc7o6)nc5)ncc34)nc(N3CCOCC3)cc2n(C)c1=O] | 5-(8-(1,3-dimethyl-7-morpholino-2-oxo-1,2- dihydro-1,6-naphthyridin-5-yl)-6-methyl-5- oxo-5,6-dihydro-2,6-naphthyridin-3-yl)-N-(3- (4-(2,6-dioxopiperidin-3-yl)benzofuran-2-yl) prop-2-yn-1-yl)picolinamide |
| B132 | 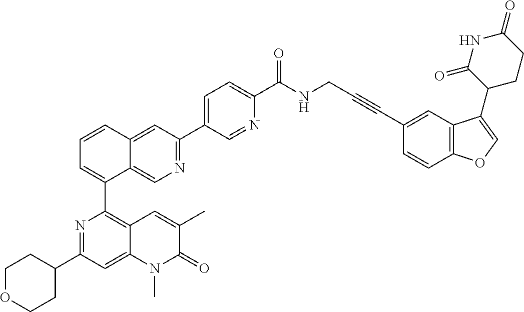 [SMILES: Cc1cc2c(-c3cccc4cc(-c5ccc(C(=O)NCC#Cc6ccc7occ(C8CCC(=O)NC8=O)c7c6)nc5)ncc34)nc(C3CCOCC3)cc2n(C)c1=O] | 5-(8-(1,3-dimethyl-2-oxo-7-(tetrahydro-2H- pyran-4-yl)-1,2-dihydro-1,6-naphthyridin-5- yl)isoquinolin-3-yl)-N-(3-(3-(2,6-dioxo- piperidin-3-yl)benzofuran-5-yl)prop-2-yn-1- yl)picolinamide |
| B133 | 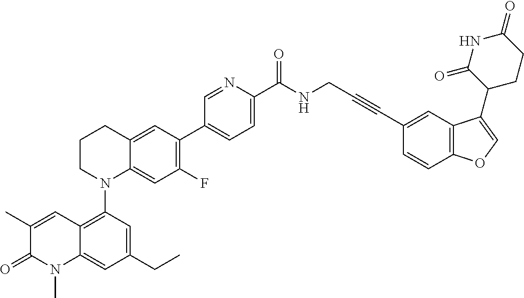 [SMILES: CCc1cc(N2CCCc3cc(-c4ccc(C(=O)NCC#Cc5ccc6occ(C7CCC(=O)NC7=O)c6c5)nc4)c(F)cc32)c2cc(C)c(=O)n(C)c2c1] | N-(3-(3-(2,6-dioxopiperidin-3-yl)benzofuran- 5-yl)prop-2-yn-1-yl)-5-(7′-ethyl-7-fluoro-1′,3′- dimethyl-2′-oxo-1′,2′,3,4-tetrahydro-2H- [1,5′-biquinolin]-6-yl)picolinamide |
| B134 | 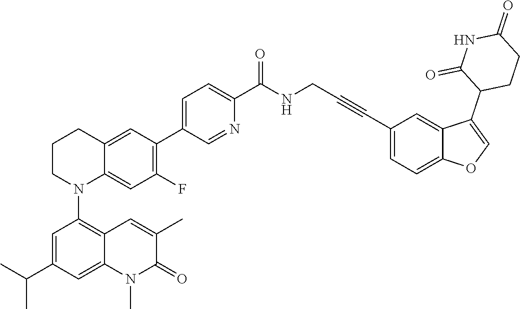 [SMILES: Cc1cc2c(N3CCCc4cc(-c5ccc(C(=O)NCC#Cc6ccc7occ(C8CCC(=O)NC8=O)c7c6)nc5)c(F)cc43)cc(C(C)C)cc2n(C)c1=O] | N-(3-(3-(2,6-dioxopiperidin-3-yl)benzofuran-5- yl)prop-2-yn-1-yl)-5-(7-fluoro-7′-isopropyl- 1′,3′-dimethyl-2′-oxo-1′,2′,3,4-tetrahydro-2H- [1,5′-biquinolin]-6-yl)picolinamide |
| B135 | 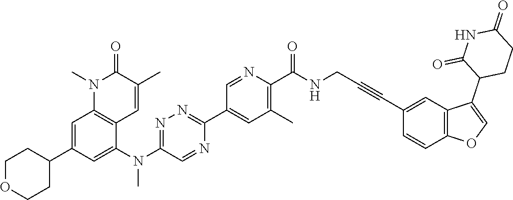 [SMILES: Cc1cc(-c2ncc(N(C)c3cc(C4CCOCC4)cc4c3cc(C)c(=O)n4C)nn2)cnc1C(=O)NCC#Cc1ccc2occ(C3CCC(=O)NC3=O)c2c1] | 5-(6-((1,3-dimethyl-2-oxo-7-(tetrahydro-2H- pyran-4-yl)-1,2-dihydroquinolin-5-yl) (methyl)amino)-1,2,4-triazin-3-yl)-N-(3-(3- (2,6-dioxopiperidin-3-yl)benzofuran-5-yl) prop-2-yn-1-yl)-3-methylpicolinamide |
| B136 | 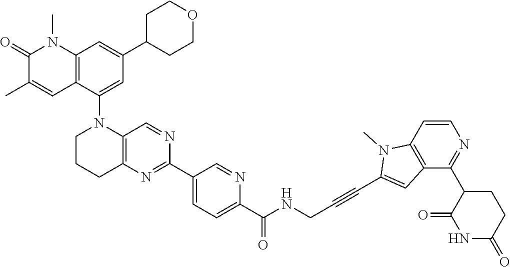 [SMILES: Cc1cc2c(N3CCCc4nc(-c5ccc(C(=O)NCC#Cc6cc7c(C8CCC(=O)NC8=O)nccc7n6C)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,6-dioxopiperidin-3-yl)-1-methyl-1H- pyrrolo[3,2-c]pyridin-2-yl)prop-2-yn-1-yl) picolinamide |
| B137 | 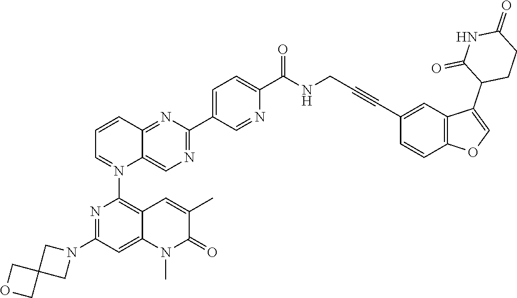 | 5-(5-(1,3-dimethyl-2-oxo-7-(2-oxa-6- azaspiro[3.3]heptan-6-yl)-1,2-dihydro-1,6- naphthyridin-5-yl)-5,6,7,8-tetrahydropyrido [3,2-d]pyrimidin-2-yl)-N-(3-(3-(2,6-dioxo- piperidin-3-yl)benzofuran-5-yl)prop-2-yn-1- yl)picolinamide |
| B138 | 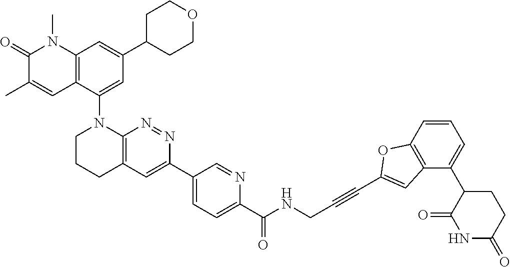 [SMILES: Cc1cc2c(N3CCCc4cc(-c5ccc(C(=O)NCC#Cc6cc7c(C8CCC(=O)NC8=O)cccc7o6)nc5)nnc43)cc(C3CCOCC3)cc2n(C)c1=O] | 5-(8-(1,3-dimethyl-2-oxo-7-(tetrahydro-2H- pyran-4-yl)-1,2-dihydroquinolin-5-yl)-5,6,7,8- tetrahydropyrido[2,3-c]pyridazin-3-yl)-N-(3- (4-(2,6-dioxopiperidin-3-yl)benzofuran-2-yl) prop-2-yn-1-yl)picolinamide |
| B139 | 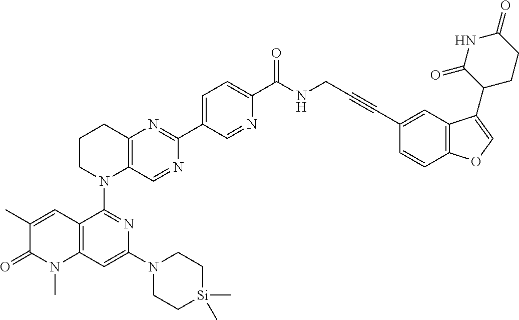 [SMILES: Cc1cc2c(N3CCCc4nc(-c5ccc(C(=O)NCC#Cc6ccc7occ(C8CCC(=O)NC8=O)c7c6)nc5)ncc43)nc(N3CC[Si](C)(C)CC3)cc2n(C)c1=O] | 5-(5-(7-(4,4-dimethyl-1,4-azasilinan-1-yl)-1,3- dimethyl-2-oxo-1,2-dihydro-1,6-naphthyridin- 5-yl)-5,6,7,8-tetrahydropyrido[3,2-d]pyrimidin- 2-yl)-N-(3-(3-(2,6-dioxopiperidin-3-yl) benzofuran-5-yl)prop-2-yn-1-yl)picolinamide |
| B140 | 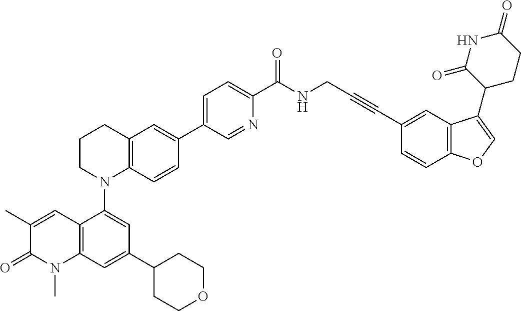 [SMILES: Cc1cc2c(N3CCCc4cc(-c5ccc(C(=O)NCC#Cc6ccc7occ(C8CCC(=O)NC8=O)c7c6)nc5)ccc43)cc(C3CCOCC3)cc2n(C)c1=O] | 5-(1′,3′-dimethyl-2′-oxo-7′-(tetrahydro-2H- pyran-4-yl)-1′,2′,3,4-tetrahydro-2H-[1,5′- biquinolin]-6-yl)-N-(3-(3-(2,6-dioxopiperidin- 3-yl)benzofuran-5-yl)prop-2-yn-1-yl) picolinamide |
| B141 | 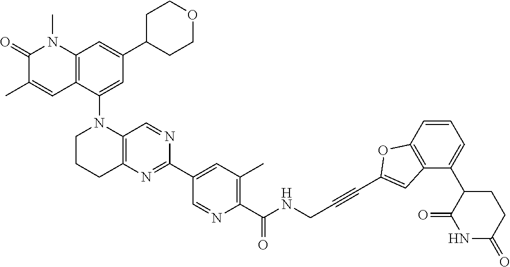 [SMILES: Cc1cc(-c2ncc3c(n2)CCCN3c2cc(C3CCOCC3)cc3c2cc(C)c(=O)n3C)cnc1C(=O)NCC#Cc1cc2c(C3CCC(=O)NC3=O)cccc2o1] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,6-dioxopiperidin-3-yl)benzofuran-2-yl) prop-2-yn-1-yl)-3-methylpicolinamide |
| B142 | 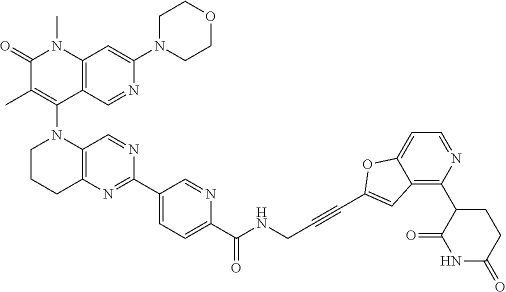 [SMILES: Cc1c(N2CCCc3nc(-c4ccc(C(=O)NCC#Cc5cc6c(C7CCC(=O)NC7=O)nccc6o5)nc4)ncc32)c2cnc(N3CCOCC3)cc2n(C)c1=O] | 5-(5-(1,3-dimethyl-7-morpholino-2-oxo-1,2- dihydro-1,6-naphthyridin-5-yl)-5,6,7,8- tetrahydropyrido[3,2-d]pyrimidin-2-yl)-N- (3-(4-(2,6-dioxopiperidin-3-yl)furo[3,2-c] pyridin-2-yl)prop-2-yn-1-yl)picolinamide |
| B143 | 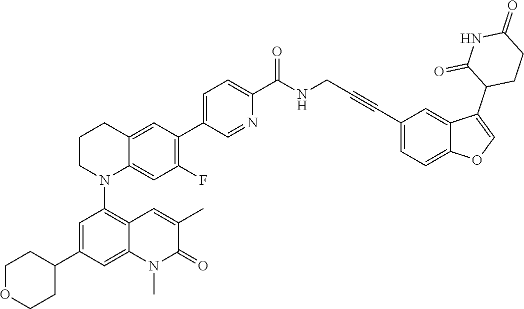 [SMILES: Cc1cc2c(N3CCCc4cc(-c5ccc(C(=O)NCC#Cc6ccc7occ(C8CCC(=O)NC8=O)c7c6)nc5)c(F)cc43)cc(C3CCOCC3)cc2n(C)c1=O] | N-(3-(3-(2,6-dioxopiperidin-3-yl)benzofuran-5- yl)prop-2-yn-1-yl)-5-(7-fluoro-1′,3′-dimethyl- 2′-oxo-7′-(tetrahydro-2H-pyran-4-yl)-1′,2′,3,4- tetrahydro-2H-[1,5′-biquinolin]-6-yl) picolinamide |
| B144 | 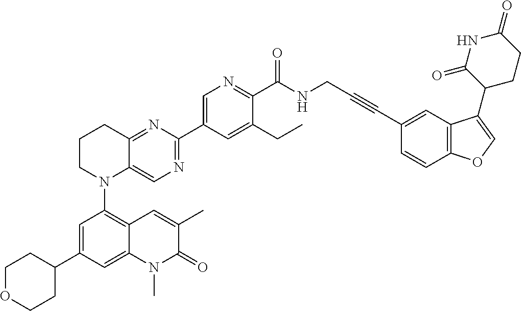 [SMILES: CCc1cc(-c2ncc3c(n2)CCCN3c2cc(C3CCOCC3)cc3c2cc(C)c(=O)n3C)cnc1C(=O)NCC#Cc1ccc2occ(C3CCC(=O)NC3=O)c2c1] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (3-(2,6-dioxopiperidin-3-yl)benzofuran-5-yl) prop-2-yn-1-yl)-3-ethylpicolinamide |
| B145 | 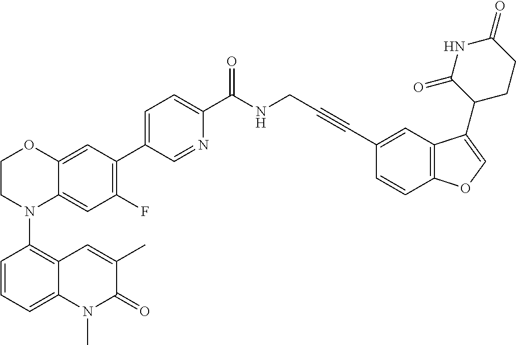 [SMILES: Cc1cc2c(N3CCOc4cc(-c5ccc(C(=O)NCC#Cc6ccc7occ(C8CCC(=O)NC8=O)c7c6)nc5)c(F)cc43)cccc2n(C)c1=O] | 5-(4-(1,3-dimethyl-2-oxo-1,2-dihydroquinolin- 5-yl)-6-fluoro-3,4-dihydro-2H-benzo[b][1,4] oxazin-7-yl)-N-(3-(3-(2,6-dioxopiperidin-3- yl)benzofuran-5-yl)prop-2-yn-1-yl) picolinamide |
| B146 | 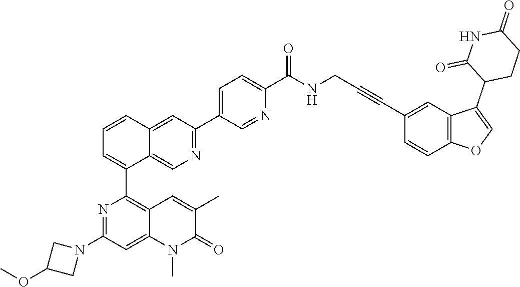 [SMILES: COC1CN(c2cc3c(cc(C)c(=O)n3C)c(-c3cccc4cc(-c5ccc(C(=O)NCC#Cc6ccc7occ(C8CCC(=O)NC8=O)c7c6)nc5)ncc34)n2)C1] | N-(3-(3-(2,6-dioxopiperidin-3-yl)benzofuran-5- yl)prop-2-yn-1-yl)-5-(8-(7-(3-methoxyazetidin- 1-yl)-1,3-dimethyl-2-oxo-1,2-dihydro-1,6- naphthyridin-5-yl)isoquinolin-3-yl) picolinamide |
| B147 | 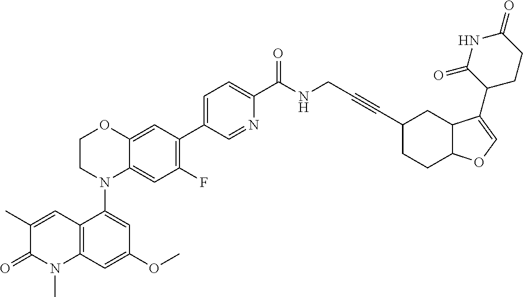 [SMILES: COc1cc(N2CCOc3cc(-c4ccc(C(=O)NCC#CC5CCC6OC=C(C7CCC(=O)NC7=O)C6C5)nc4)c(F)cc32)c2cc(C)c(=O)n(C)c2c1] | N-(3-(3-(2,6-dioxopiperidin-3-yl)benzofuran- 5-yl)prop-2-yn-1-yl)-5-(6-fluoro-4-(7- methoxy-1,3-dimethyl-2-oxo-1,2-dihydro- quinolin-5-yl)-3,4-dihydro-2H-benzo[b][1,4] oxazin-7-yl)picolinamide |
| B148 | 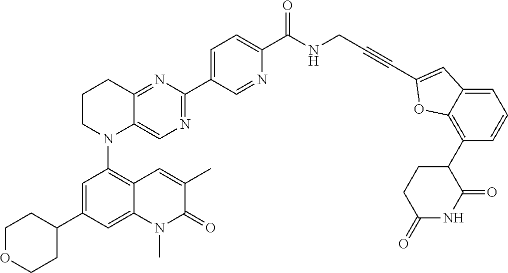 [SMILES: Cc1cc2c(N3CCCc4nc(-c5ccc(C(=O)NCC#Cc6cc7cccc(C8CCC(=O)NC8=O)c7o6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (7-(2,6-dioxopiperidin-3-yl)benzofuran-2-yl) prop-2-yn-1-yl)picolinamide |
| B149 | 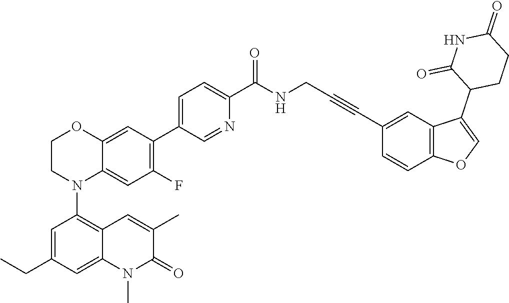 [SMILES: CCc1cc(N2CCOc3cc(-c4ccc(C(=O)NCC#Cc5ccc6occ(C7CCC(=O)NC7=O)c6c5)nc4)c(F)cc32)c2cc(C)c(=O)n(C)c2c1] | N-(3-(3-(2,6-dioxopiperidin-3-yl)benzofuran- 5-yl)prop-2-yn-1-yl)-5-(4-(7-ethyl-1,3- dimethyl-2-oxo-1,2-dihydroquinolin-5-yl)-6- fluoro-3,4-dihydro-2H-benzo[b][1,4] oxazin-7-yl)picolinamide |
| B150 | 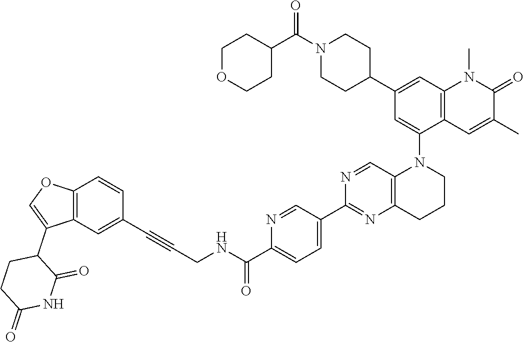 [SMILES: Cc1cc2c(N3CCCc4nc(-c5ccc(C(=O)NCC#Cc6ccc7occ(C8CCC(=O)NC8=O)c7c6)nc5)ncc43)cc(C3CCN(C(=O)C4CCOCC4)CC3)cc2n(C)c1=O] | 5-(5-(1,3-dimethyl-2-oxo-7-(1-(tetrahydro-2H- pyran-4-carbonyl)piperidin-4-yl)-1,2-dihydro- quinolin-5-yl)-5,6,7,8-tetrahydropyrido[3,2-d] pyrimidin-2-yl)-N-(3-(3-(2,6-dioxopiperidin- 3-yl)benzofuran-5-yl)prop-2-yn-1-yl) picolinamide |
| B151 | 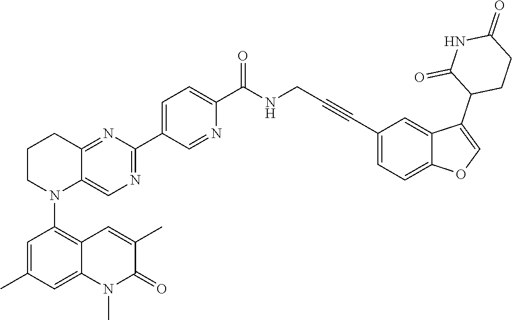 [SMILES: Cc1cc(N2CCCc3nc(-c4ccc(C(=O)NCC#Cc5ccc6occ(C7CCC(=O)NC7=O)c6c5)nc4)ncc32)c2cc(C)c(=O)n(C)c2c1] | N-(3-(3-(2,6-dioxopiperidin-3-yl)benzofuran-5- yl)prop-2-yn-1-yl)-5-(5-(1,3,7-trimethyl-2-oxo- 1,2-dihydroquinolin-5-yl)-5,6,7,8-tetrahydro- pyrido[3,2-d]pyrimidin-2-yl)picolinamide |
| B152 | 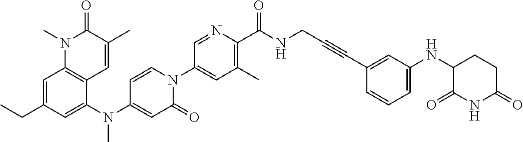 [SMILES: CCc1cc(N(C)c2ccn(-c3cnc(C(=O)NCC#Cc4cccc(NC5CCC(=O)NC5=O)c4)c(C)c3)c(=O)c2)c2cc(C)c(=O)n(C)c2c1] | N-(3-(3-((2,6-dioxopiperidin-3-yl)amino) phenyl)prop-2-yn-1-yl)-4-((7-ethyl-1,3- dimethyl-2-oxo-1,2-dihydroquinolin-5-yl) (methyl)amino)-5′-methyl-2-oxo-2H-[1,3′- bipyridine]-6′-carboxamide |
| B153 | 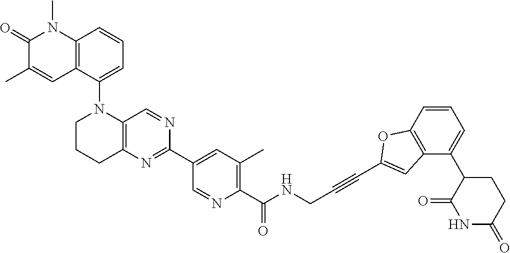 [SMILES: Cc1cc(-c2ncc3c(n2)CCCN3c2cccc3c2cc(C)c(=O)n3C)cnc1C(=O)NCC#Cc1cc2c(C3CCC(=O)NC3=O)cccc2o1] | 5-(5-(1,3-dimethyl-2-oxo-1,2-dihydroquinolin- 5-yl)-5,6,7,8-tetrahydropyrido[3,2-d] pyrimidin-2-yl)-N-(3-(4-(2,6-dioxopiperidin- 3-yl)benzofuran-2-yl)prop-2-yn-1-yl)-3- methylpicolinamide |
| B154 | 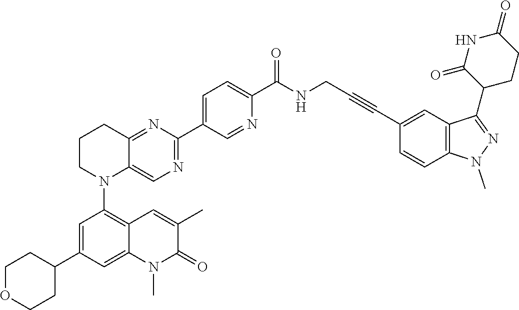 [SMILES: Cc1cc2c(N3CCCc4nc(-c5ccc(C(=O)NCC#Cc6ccc7c(c6)c(C6CCC(=O)NC6=O)nn7C)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (3-(2,6-dioxopiperidin-3-yl)-1-methyl-1H- indazol-5-yl)prop-2-yn-1-yl)picolinamide |
| B155 | 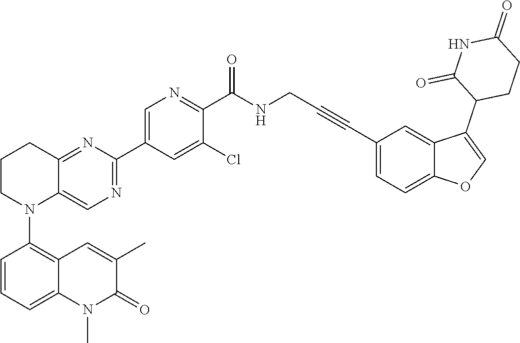 [SMILES: Cc1cc2c(N3CCCc4nc(-c5cnc(C(=O)NCC#Cc6ccc7occ(C8CCC(=O)NC8=O)c7c6)c(Cl)c5)ncc43)cccc2n(C)c1=O] | 3-chloro-5-(5-(1,3-dimethyl-2-oxo-1,2- dihydroquinolin-5-yl)-5,6,7,8-tetrahydro- pyrido[3,2-d]pyrimidin-2-yl)-N-(3-(3-(2,6- dioxopiperidin-3-yl)benzofuran-5-yl)prop-2- yn-1-yl)picolinamide |
| B156 | 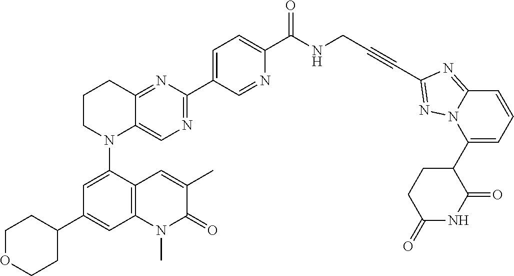 [SMILES: Cc1cc2c(N3CCCc4nc(-c5ccc(C(=O)NCC#Cc6nc7cccc(C8CCC(=O)NC8=O)n7n6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (5-(2,6-dioxopiperidin-3-yl)-[1,2,4]triazolo [1,5-a]pyridin-2-yl)prop-2-yn-1-yl) picolinamide |
| B157 | 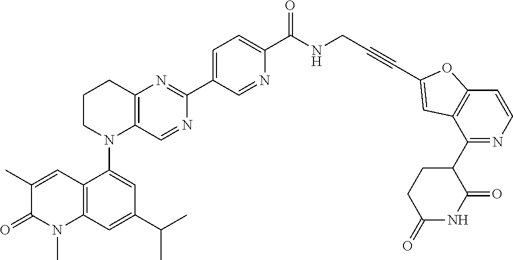 [SMILES: Cc1cc2c(N3CCCc4nc(-c5ccc(C(=O)NCC#Cc6cc7c(C8CCC(=O)NC8=O)nccc7o6)nc5)ncc43)cc(C(C)C)cc2n(C)c1=O] | N-(3-(4-(2,6-dioxopiperidin-3-yl)furo[3,2-c] pyridin-2-yl)prop-2-yn-1-yl)-5-(5-(7-isopropyl- 1,3-dimethyl-2-oxo-1,2-dihydroquinolin-5-yl)- 5,6,7,8-tetrahydropyrido[3,2-d]pyrimidin-2-yl) picolinamide |
| B158 | 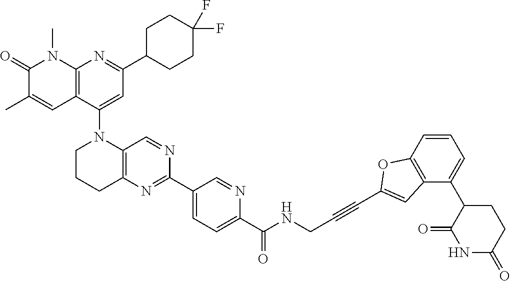 [SMILES: Cc1cc2c(N3CCCc4nc(-c5ccc(C(=O)NCC#Cc6cc7c(C8CCC(=O)NC8=O)cccc7o6)nc5)ncc43)cc(C3CCC(F)(F)CC3)nc2n(C)c1=O] | 5-(5-(2-(4,4-difluorocyclohexyl)-6,8-dimethyl- 7-oxo-7,8-dihydro-1,8-naphthyridin-4-yl)- 5,6,7,8-tetrahydropyrido[3,2-d]pyrimidin-2-yl)- N-(3-(4-(2,6-dioxopiperidin-3-yl)benzofuran- 2-yl)prop-2-yn-1-yl)picolinamide |
| B159 | 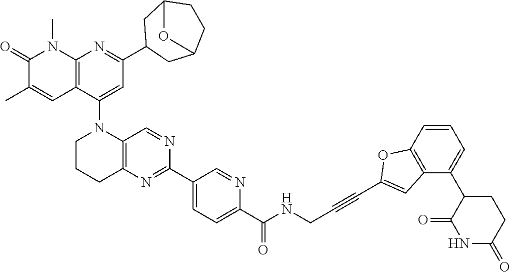 [SMILES: Cc1cc2c(N3CCCc4nc(-c5ccc(C(=O)NCC#Cc6cc7c(C8CCC(=O)NC8=O)cccc7o6)nc5)ncc43)cc(C3CC4CCC(C3)O4)nc2n(C)c1=O] | 5-(5-(2-(8-oxabicyclo[3.2.1]octan-3-yl)-6,8- dimethyl-7-oxo-7,8-dihydro-1,8-naphthyridin- 4-yl)-5,6,7,8-tetrahydropyrido[3,2-d]pyrimidin- 2-yl)-N-(3-(4-(2,6-dioxopiperidin-3-yl) benzofuran-2-yl)prop-2-yn-1-yl)picolinamide |
| B160 | 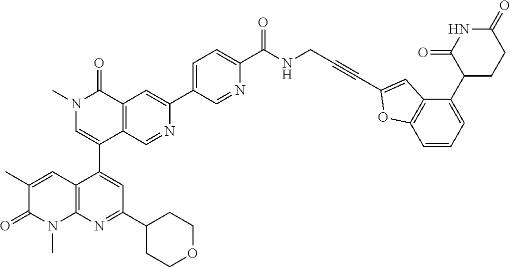 [SMILES: Cc1cc2c(-c3cn(C)c(=O)c4cc(-c5ccc(C(=O)NCC#Cc6cc7c(C8CCC(=O)NC8=O)cccc7o6)nc5)ncc34)cc(C3CCOCC3)nc2n(C)c1=O] | 5-(8-(6,8-dimethyl-7-oxo-2-(tetrahydro-2H- pyran-4-yl)-7,8-dihydro-1,8-naphthyridin-4-yl)- 6-methyl-5-oxo-5,6-dihydro-2,6-naphthyridin- 3-yl)-N-(3-(4-(2,6-dioxopiperidin-3-yl) benzofuran-2-yl)prop-2-yn-1-yl)picolinamide |
| B161 | 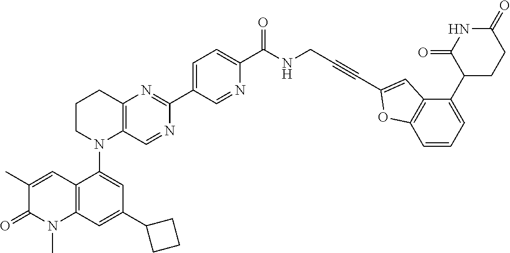 [SMILES: Cc1cc2c(N3CCCc4nc(-c5ccc(C(=O)NCC#Cc6cc7c(C8CCC(=O)NC8=O)cccc7o6)nc5)ncc43)cc(C3CCC3)cc2n(C)c1=O] | 5-(5-(7-cyclobutyl-1,3-dimethyl-2-oxo-1,2- dihydroquinolin-5-yl)-5,6,7,8-tetrahydropyrido [3,2-d]pyrimidin-2-yl)-N-(3-(4-(2,6-dioxo- piperidin-3-yl)benzofuran-2-yl)prop-2-yn-1- yl)picolinamide |
| B162 | 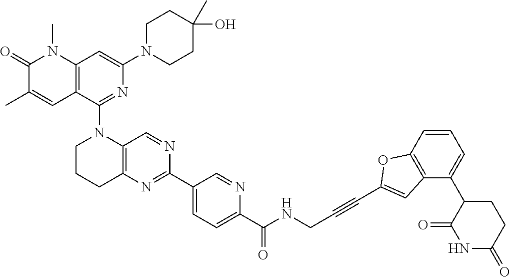 [SMILES: Cc1cc2c(N3CCCc4nc(-c5ccc(C(=O)NCC#Cc6cc7c(C8CCC(=O)NC8=O)cccc7o6)nc5)ncc43)nc(N3CCC(C)(O)CC3)cc2n(C)c1=O] | N-(3-(4-(2,6-dioxopiperidin-3-yl)benzofuran-2- yl)prop-2-yn-1-yl)-5-(5-(7-(4-hydroxy-4- methylpiperidin-1-yl)-1,3-dimethyl-2-oxo-1,2- dihydro-1,6-naphthyridin-5-yl)-5,6,7,8- tetrahydropyrido[3,2-d]pyrimidin-2-yl) picolinamide |
| B163 | 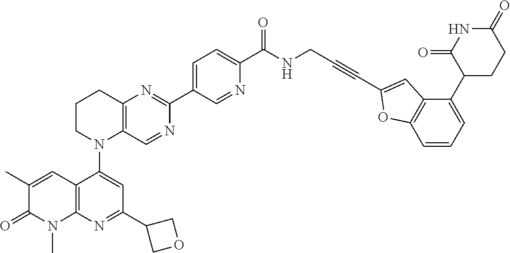 [SMILES: Cc1cc2c(N3CCCc4nc(-c5ccc(C(=O)NCC#Cc6cc7c(C8CCC(=O)NC8=O)cccc7o6)nc5)ncc43)cc(C3COC3)nc2n(C)c1=O] | 5-(5-(6,8-dimethyl-2-(oxetan-3-yl)-7-oxo-7,8- dihydro-1,8-naphthyridin-4-yl)-5,6,7,8- tetrahydropyrido[3,2-d]pyrimidin-2-yl)-N-(3- (4-(2,6-dioxopiperidin-3-yl)benzofuran-2-yl) prop-2-yn-1-yl)picolinamide |
| B164 | 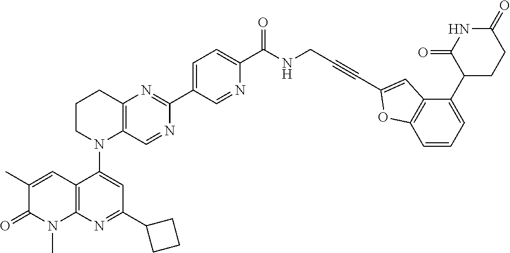 [SMILES: Cc1cc2c(N3CCCc4nc(-c5ccc(C(=O)NCC#Cc6cc7c(C8CCC(=O)NC8=O)cccc7o6)nc5)ncc43)cc(C3CCC3)nc2n(C)c1=O] | 5-(5-(2-cyclobutyl-6,8-dimethyl-7-oxo-7,8- dihydro-1,8-naphthyridin-4-yl)-5,6,7,8-tetra- hydropyrido[3,2-d]pyrimidin-2-yl)-N-(3-(4- (2,6-dioxopiperidin-3-yl)benzofuran-2-yl)prop- 2-yn-1-yl)picolinamide |
| B165 | 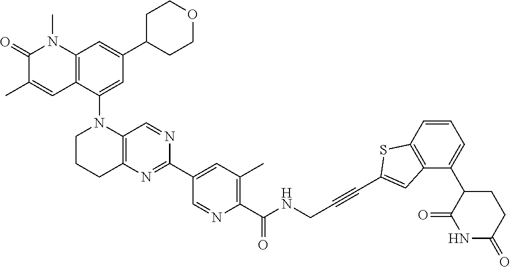 [SMILES: Cc1cc(-c2ncc3c(n2)CCCN3c2cc(C3CCOCC3)cc3c2cc(C)c(=O)n3C)cnc1C(=O)NCC#Cc1cc2c(C3CCC(=O)NC3=O)cccc2s1] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,6-dioxopiperidin-3-yl)benzo[b]thiophen- 2-yl)prop-2-yn-1-yl)-3-methylpicolinamide |
| B166 | 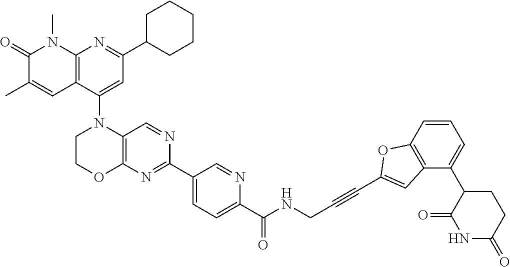 [SMILES: Cc1cc2c(N3CCOc4nc(-c5ccc(C(=O)NCC#Cc6cc7c(C8CCC(=O)NC8=O)cccc7o6)nc5)ncc43)cc(C3CCCCC3)nc2n(C)c1=O] | 5-(5-(6,8-dimethyl-7-oxo-2-(tetrahydro-2H- pyran-4-yl)-7,8-dihydro-1,8-naphthyridin-4- yl)-6,7-dihydro-5H-pyrimido[4,5-b][1,4] oxazin-2-yl)-N-(3-(4-(2,6-dioxopiperidin-3- yl)benzofuran-2-yl)prop-2-yn-1-yl) picolinamide |
| B167 | 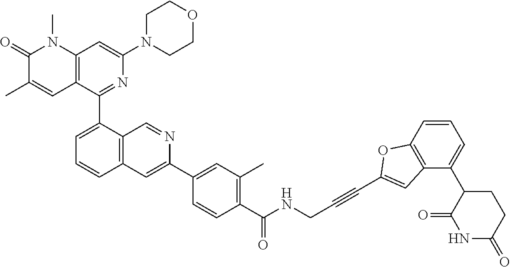 [SMILES: Cc1cc(-c2cc3cccc(-c4nc(N5CCOCC5)cc5c4cc(C)c(=O)n5C)c3cn2)ccc1C(=O)NCC#Cc1cc2c(C3CCC(=O)NC3=O)cccc2o1] | 5-(8-(1,3-dimethyl-7-morpholino-2-oxo-1,2- dihydro-1,6-naphthyridin-5-yl)isoquinolin-3- yl)-N-(3-(4-(2,6-dioxopiperidin-3-yl) benzofuran-2-yl)prop-2-yn-1-yl)-3- methylpicolinamide |
| B168 | 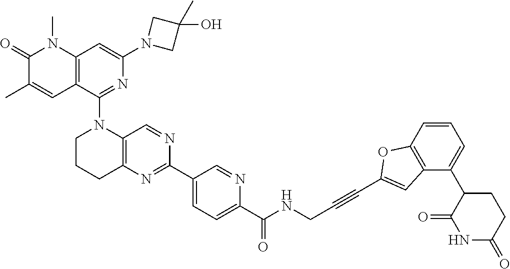 [SMILES: Cc1cc2c(N3CCCc4nc(-c5ccc(C(=O)NCC#Cc6cc7c(C8CCC(=O)NC8=O)cccc7o6)nc5)ncc43)nc(N3CC(C)(O)C3)cc2n(C)c1=O] | N-(3-(4-(2,6-dioxopiperidin-3-yl)benzofuran- 2-yl)prop-2-yn-1-yl)-5-(5-(7-(3-hydroxy-3- methylazetidin-1-yl)-1,3-dimethyl-2-oxo-1,2- dihydro-1,6-naphthyridin-5-yl)-5,6,7,8- tetrahydropyrido[3,2-d]pyrimidin-2-yl) picolinamide |
| B169 | 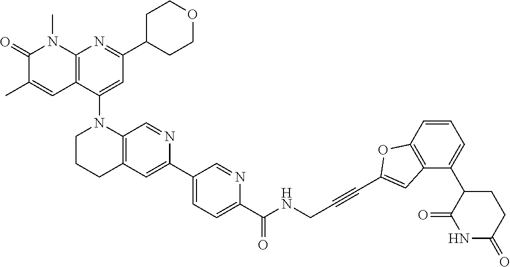 [SMILES: Cc1cc2c(N3CCCc4cc(-c5ccc(C(=O)NCC#Cc6cc7c(C8CCC(=O)NC8=O)cccc7o6)nc5)ncc43)cc(C3CCOCC3)nc2n(C)c1=O] | 5-(1-(6,8-dimethyl-7-oxo-2-(tetrahydro-2H- pyran-4-yl)-7,8-dihydro-1,8-naphthyridin-4- yl)-1,2,3,4-tetrahydro-1,7-naphthyridin-6-yl)- N-(3-(4-(2,6-dioxopiperidin-3-yl)benzofuran- 2-yl)prop-2-yn-1-yl)picolinamide |
| B170 | 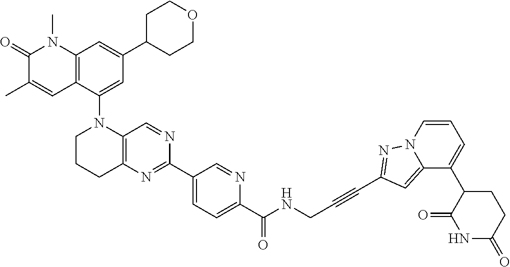 [SMILES: Cc1cc2c(N3CCCc4nc(-c5ccc(C(=O)NCC#Cc6cc7c(C8CCC(=O)NC8=O)cccn7n6)nc5)ncc43)cc(C3CCOCC3)cc2n(C)c1=O] | 5-(5-(1,3-dimethyl-2-oxo-7-(tetrahydro-2H- pyran-4-yl)-1,2-dihydroquinolin-5-yl)-5,6,7,8- tetrahydropyrido[3,2-d]pyrimidin-2-yl)-N-(3- (4-(2,6-dioxopiperidin-3-yl)pyrazolo[1,5-a] pyridin-2-yl)prop-2-yn-1-yl)picolinamide |
| B171 | 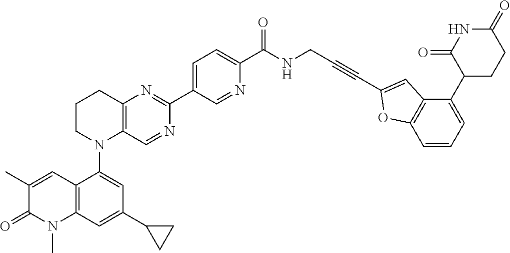 [SMILES: Cc1cc2c(N3CCCc4nc(-c5ccc(C(=O)NCC#Cc6cc7c(C8CCC(=O)NC8=O)cccc7o6)nc5)ncc43)cc(C3CC3)cc2n(C)c1=O] | 5-(5-(7-cyclopropyl-1,3-dimethyl-2-oxo-1,2- dihydroquinolin-5-yl)-5,6,7,8-tetrahydropyrido [3,2-d]pyrimidin-2-yl)-N-(3-(4-(2,6-dioxo- piperidin-3-yl)benzofuran-2-yl)prop-2-yn-1- yl)picolinamide |
| B172 | 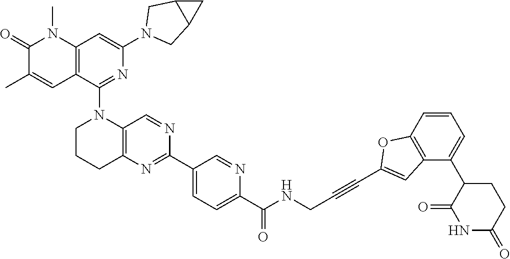 [SMILES: Cc1cc2c(N3CCCc4nc(-c5ccc(C(=O)NCC#Cc6cc7c(C8CCC(=O)NC8=O)cccc7o6)nc5)ncc43)nc(N3CC4CC4C3)cc2n(C)c1=O] | 5-(5-(7-(3-azabicyclo[3.1.0]hexan-3-yl)-1,3- dimethyl-2-oxo-1,2-dihydro-1,6-naphthyridin- 5-yl)-5,6,7,8-tetrahydropyrido[3,2-d]pyrimidin- 2-yl)-N-(3-(4-(2,6-dioxopiperidin-3-yl) benzofuran-2-yl)prop-2-yn-1-yl)picolinamide |

TABLE 3

| Compound | | |
|---|---|---|
| No. | Chemical Structure | Chemical Name |
| C1 | 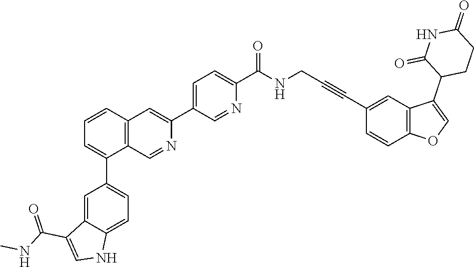 [SMILES: CNC(=O)c1c[nH]c2ccc(-c3cccc4cc(-c5ccc(C(=O)NCC#Cc6ccc7occ(C8CCC(=O)NC8=O)c7c6)nc5)ncc34)cc12] | 5-(3-(6-((3-(3-(2,6-dioxopiperidin-3-yl)benzofuran- 5-yl)prop-2-yn-1-yl)carbamoyl)pyridin-3-yl) isoquinolin-8-yl)-N-methyl-1H-indole-3- carboxamide |

TABLE 4

| Compound | | |
|---|---|---|
| No. | Chemical Structure | Chemical Name |
| D1 | 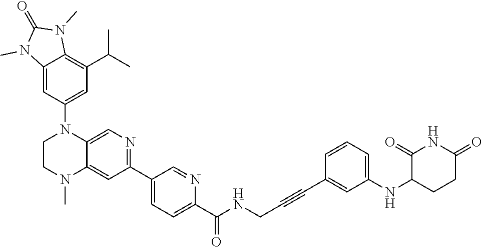 [SMILES: CC(C)c1cc(N2CCN(C)c3cc(-c4ccc(C(=O)NCC#Cc5cccc(NC6CCC(=O)NC6=O)c5)nc4)ncc32)cc2c1n(C)c(=O)n2C] | N-(3-(3-((2,6-dioxopiperidin-3- yl)amino)phenyl)prop-2-yn-1-yl)-5- (4-(7-isopropyl-1,3-dimethyl-2-oxo- 2,3-dihydro-1H-benzo[d]imidazol-5- yl)-1-methyl-1,2,3,4-tetrahydropyrido [3,4-b]pyrazin-7-yl)picolinamide |
| D2 | 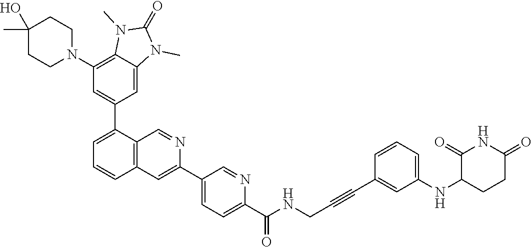 [SMILES: Cn1c(=O)n(C)c2c(N3CCC(C)(O)CC3)cc(-c3cccc4cc(-c5ccc(C(=O)NCC#Cc6cccc(NC7CCC(=O)NC7=O)c6)nc5)ncc34)cc21] | N-(3-(3-((2,6-dioxopiperidin-3- yl)amino)phenyl)prop-2-yn-1-yl)-5- (8-(7-(4-hydroxy-4-methylpiperidin- 1-yl)-1,3-dimethyl-2-oxo-2,3-dihydro- 1H-benzo[d]imidazol-5-yl)isoquinolin- 3-yl)picolinamide |

In certain embodiments, the compound is selected from Compound Nos. A169, A170, A173, A174, A181, A189, A259, B28, B37, B38, B47, B48, B74, and B163 and pharmaceutically acceptable salts thereof.

In certain embodiments, the compound is selected from Compound Nos. A169, A170, A173, A174, A181, A189, A259, B28, B37, B38, B47, B48, B74, and B163.

In certain embodiments, the compound is selected from Compound Nos. A169, A170, A173, A174, A181, A189, and A259 and pharmaceutically acceptable salts thereof.

In certain embodiments, the compound is selected from Compound Nos. A169, A170, A173, A174, A181, A189, and A259.

In certain embodiments, the compound is selected from Compound Nos. B28, B37, B38, B47, B48, B74, and B163 and pharmaceutically acceptable salts thereof.

In certain embodiments, the compound is selected from Compound NosB28, B37, B38, B47, B48, B74, and B163.

To date, heterobifunctional protein degraders carry both opportunities and challenges. The biggest challenge is that, unlike traditional small-molecule drugs, degraders do not meet the classic “rule of 5” (RO5) which was proposed by Christopher A. Lipinski in 1997: molecular weight (MW)<500 kDa; solubility (LogP)<5; H-bond donors (HBDs)<5; H-bond acceptors (HBAs)<10; and number of rotatable bonds (nRotB)<10. Compounds conforming to Lipinski's rule have a higher probability of possessing good pharmacokinetic (PK) properties and high oral bioavailability compared with compounds that don't satisfy the RO5. Although heterobifunctional degraders are considered small-molecule compounds, they typically violate the RO5 due to molecular weight >500 kDa and several other RO5 parameters above the cutoffs. As a result, poor oral absorption is a common and major problem for heterobifunctional degraders.

In order to screen for degraders with acceptable oral bioavailability, parameters such as molecular weight, solubility, topological polar surface area (tPSA), H-bond donor, H-bond acceptor, number of rotatable bonds, as well as metabolic stability/permeability, need to be carefully characterized and optimized. In addition, selectivity off-target proteins needs to be optimized as well. In fact, many current studies only demonstrate the effectiveness and anti-proliferative activity of the designed degraders on target protein degradation at the cellular levels, with acknowledgement of the challenge of achieving orally bioavailable compounds.

The present disclosure is based, at least in part, on the discovery that the compounds disclosed herein may possess improved oral bioavailability relative to other p300 degraders.

The compounds of the present disclosure may also possess advantageous characteristics, as compared to other p300 degraders. For example, the compounds of the present disclosure may potentially show selectivity for p300 over CBP, may display more potent degradation activity against p300, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction).

In certain embodiments, a compound disclosed herein shows selective degradation of p300 over CBP when a compound has a DC₅₀ for p300 that is lower than its DC₅₀ for CBP when both values are measured at the same time point. In certain embodiments, a compound disclosed herein shows selectivity when a compound has a Dₘₐₓ for p300 that is greater than its Dₘₐₓ for CBP when both values are measured at the same time point. In certain embodiments, a compound disclosed herein shows selectivity through a combination of both lower DC₅₀ and greater Dₘₐₓ for p300, as compared to those for CBP. In certain embodiments, a compound disclosed herein shows selectivity when a compound has a DC₅₀ for p300 at least 10-fold lower than its DC₅₀ for CBP and/or a value of Dₘₐₓ for p300 minus Dₘₐₓ for CBP (ΔDₘₐₓ) of at least 30, at least 35, at least 40, or at least 45 percentage points. In certain preferred embodiments, a compound disclosed herein shows selectivity when a compound has a DC₅₀ for p300 at least 30-fold lower than its DC₅₀ for CBP and/or a value of Dₘₐₓ for p300 minus Dₘₐₓ for CBP (ΔDₘₐₓ) of at least 50, at least 55, at least 60, or at least 65 percentage points. In certain more preferred embodiments, a compound disclosed herein shows selectivity when a compound has a DC₅₀ for p300 at least 100-fold lower than its DC₅₀ for CBP and/or a value of Dₘₐₓ for p300 minus Dₘₐₓ for CBP (ΔDₘₐₓ) of at least 70, at least 75, at least 80, at least 85, or at least 90 percentage points. In certain embodiments, a compound disclosed herein shows selectivity when it has a Dₘₐₓ for p300 measured at a short time point (for instance, 2 hours) that is equivalent or greater than its Dₘₐₓ for CBP measured at a significantly longer time point (for instance, 24 hours). In certain preferred embodiments, a compound has a (ΔDₘₐₓ) of at least 30, at least 40, at least 50, at least 60, at least 70, at least 80, or at least 90 percentage points even when measured stringently (at 2 hours for p300 and 24 hours for CBP). These beneficial properties of the compounds of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

### Pharmaceutically Acceptable Salts

In certain embodiments, the compounds disclosed herein exist as their pharmaceutically acceptable salts. In certain embodiments, the methods disclosed herein include methods of treating diseases by administering such pharmaceutically acceptable salts. In certain embodiments, the methods disclosed herein include methods of treating diseases by administering such pharmaceutically acceptable salts as pharmaceutical compositions.

In certain embodiments, the compounds described herein possess acidic or basic groups and therefor react with any of a number of inorganic or organic bases, and inorganic and organic acids, to form a pharmaceutically acceptable salt. In certain embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In certain embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In certain embodiments, water or oil-soluble or dispersible products are obtained by such quaternization.

### Solvates

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the disclosure may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the disclosure.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present disclosure.

In certain embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers (Stereoisomers, Geometric Isomer, Tautomer, Etc.)

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R - and S -sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is termed a “racemic mixture”.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight (S)-compound, at least about 99% by weight (R)-compound and at most about 1% by weight (S)-compound, or at least about 99.9% by weight (R)-compound and at most about 0.1% by weight (S)-compound. In certain embodiments, the weights are based upon total weight of compound.

As used herein and unless otherwise indicated, the term “enantiomerically pure (S)-compound” refers to at least about 95% by weight (S)-compound and at most about 5% by weight (R)-compound, at least about 99% by weight (S)-compound and at most about 1% by weight (R)-compound or at least about 99.9% by weight (S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure (S)-compound can comprise, for example, about 90% excipient and about 10% enantiomerically pure (S)-compound. In certain embodiments, the enantiomerically pure (S)-compound in such compositions can, for example, comprise, at least about 95% by weight (S)-compound and at most about 5% by weight (R)-compound, by total weight of the compound. In certain embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

In certain embodiments, the compounds described herein exist as geometric isomers. In certain embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the disclosure.

In certain embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the disclosure.

In additional embodiments of the compounds and methods provided herein, mixtures of enantiomers and/or diastereoisomers, resulting from a single preparative step, combination, or interconversion are useful for the applications described herein. In certain embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In certain embodiments, dissociable complexes are preferred. In certain embodiments, the diastereomers have distinct physical properties (e.g., melting points, boiling points, solubilities, reactivity, etc.) and are separated by taking advantage of these dissimilarities. In certain embodiments, the diastereomers are separated by chiral chromatography, or preferably, by separation/resolution techniques based upon differences in solubility. In certain embodiments, the optically pure enantiomer is then recovered, along with the resolving agent.

### Tautomers

In certain embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest. All tautomeric forms of the compounds disclosed herein are contemplated and are within the scope of the disclosure. The exact ratio of the tautomers depends on several factors, including temperature, solvent, and pH.

### Pharmaceutical Compositions

In certain embodiments, the compound described herein is administered as a pure chemical. In certain embodiments, the compound described herein is combined with one or more pharmaceutically suitable or acceptable carriers (also referred to herein as one or more pharmaceutically suitable (or acceptable) excipients, physiologically suitable (or acceptable) excipients, or physiologically suitable (or acceptable) carriers) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In certain embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In certain embodiments, the pharmaceutical composition is formulated for intravenous injection, oral administration, inhalation, nasal administration, topical administration, or ophthalmic administration. In certain embodiments, the pharmaceutical composition is formulated for oral administration. In certain embodiments, the pharmaceutical composition is formulated for intravenous injection. In certain embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In certain embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

### General Synthetic Scheme

A compound of formula I-1 may be prepared according to the procedures shown in SCHEME 1a, SCHEME 1b, and SCHEME 1c.

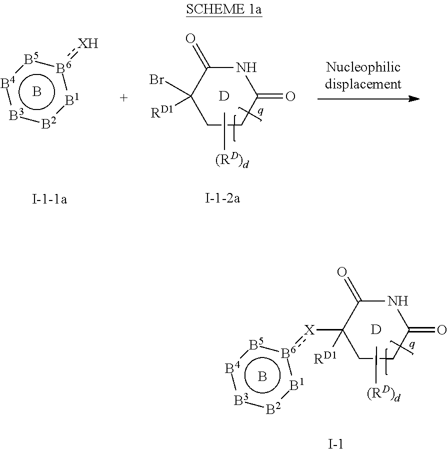

According to SCHEME 1a, commercially available or synthetically accessible substituted anilines, phenyls, phenols, thiophenols, or fused heteroaryls of formula I-1-1a are coupled with commercially available or synthetically accessible substituted bromo compounds of formula I-1-2a; with a suitable organic base such as DIPEA, K₂CO₃, NaHCO₃, or the like; in a suitable solvent such as DMF, DMSO, or the like; at temperatures ranging from 25° C. to 100° C., preferably 60° C.; to provide a cyclized compound of formula I-1.

Alternatively, a compound of formula I-1 may be prepared according to the procedures shown in SCHEME 1b.

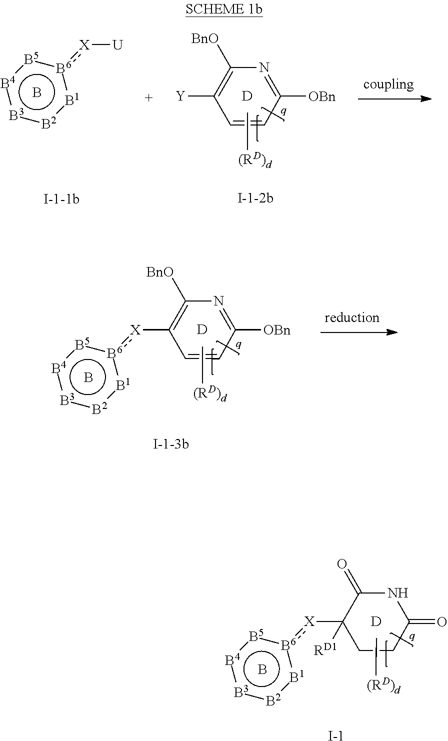

According to SCHEME 1b, commercially available or synthetically accessible substituted (hetero)arene I-1-1b (U═H, B(OR)₂, ZnCl, SnR₃, or the like) is coupled with commercially available or synthetically accessible substituted pyridine I-1-2b (Y═Cl, Br, I, OTf, or the like) according to standard Buchwald-Hartwig conditions, standard Suzuki-Miyuara conditions, standard Negishi conditions, standard Stille conditions, or the like; with a suitable palladium catalyst such as Pd(dppf)Cl₂, Pd(PPh₃)₄, Pd X-Phos G2, or the like; with or without a suitable base such as Na₂CO₃, Cs₂CO₃, K₃PO₄, KO^{t}Bu or the like; in a suitable solvent such as dioxane, THF, DMF, toluene, mixed with water or anhydrous, or the like; at temperatures ranging from 40° C. to 120° C.; to provide a coupled compound of formula I-1-3b. A bis(benzyloxy)pyridine of formula I-1-3b is then reduced with a suitable inorganic catalyst such as Pd/C, PtO₂, Pd(OH)₂, or the like; in a suitable solvent such as EtOAc, EtOH, MeOH, or the like; with a suitable hydride source such as H₂ atmosphere or the like; at temperatures ranging from 25° C. to 120° C.; to provide a compound of formula I-1.

Alternatively, a compound of formula I-1 may be prepared according to the procedures shown in SCHEME 1c.

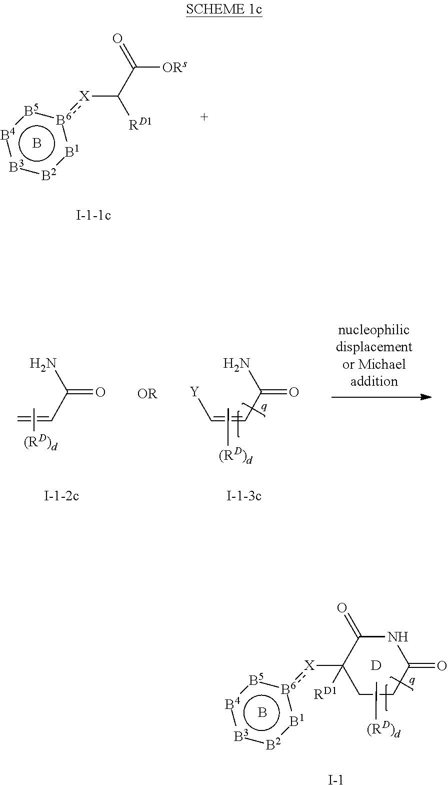

According to SCHEME 1c, a commercially available or synthetically accessible substituted (hetero)arylacetic esters of formula I-1-1c (R^{S}═Me, Et, tBu, or the like) is reacted with commercially available or synthetically accessible substituted Michael acceptors of formula I-1-2c; with or without a suitable strong base such as KO^{t}Bu or the like; in a suitable solvent such as THF or the like; at temperatures ranging from 20° C. to 120° C.; to provide a coupled compound of formula I-1. Alternatively, commercially available or synthetically accessible substituted (hetero)arylacetic esters of formula I-1-1c are reacted with commercially available or synthetically accessible substituted electrophiles of formula I-1-3c (Y═Br, I, OMs, or the like); with a suitable base such as TEA, DIPEA, NaH, or the like; in a suitable solvent such as DMF, 1,4-dioxane, or the like; at temperatures ranging 20° C. to 120° C.; to provide a coupled compound of formula I-1.

A compound of formula I-3-i may be prepared according to the procedures shown in SCHEME 2.

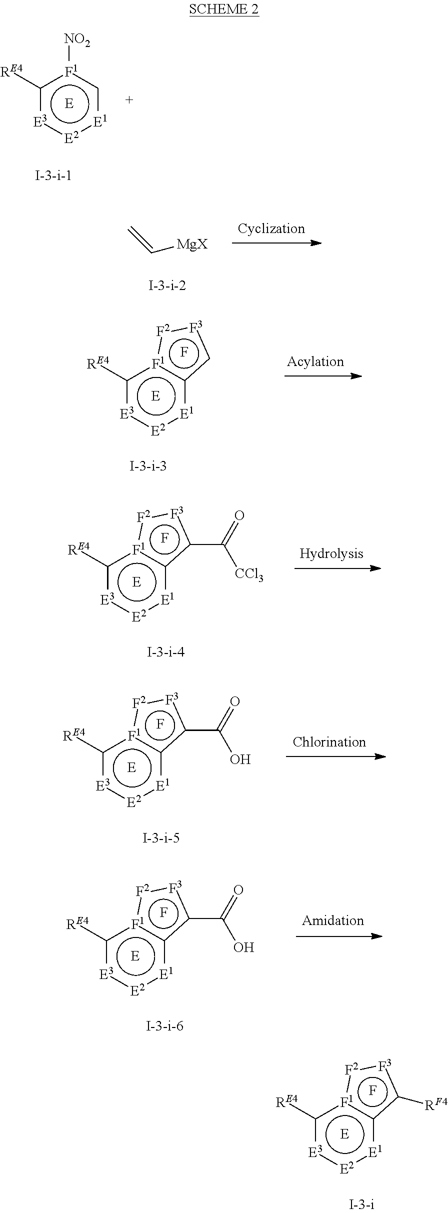

According to SCHEME 2, commercially available or synthetically accessible substituted nitro compound of formula I-3-i-1 is reacted with a vinyl Grignard agent of formula I-3-i-2 (X≡Br, Cl or I) and the like; in a suitable solvent such as THF, dioxane, and the like; at temperatures ranging from −60° C. to 25° C.; to provide cyclized compound of formula I-3-i-3. Cyclized compound of formula I-3-i-3 is acylated with commercially available 2,2,2-trichloroacetyl chloride; with a suitable Lewis acid such as AlCl₃, BF₃ etherate and the like; in a suitable solvent such as DCM, dichloroethane and the like; at temperatures ranging from 0° C. to 60° C., preferably 25° C.; to provide an acylated compound of formula I-3-i-4. Acylated compound of formula I-3-i-4 is hydrolyzed with a suitable inorganic base such as KOH, NaOH and the like; in a suitable mixed solvent such as THF, MeOH, and the like mixed with water; at temperatures ranging from 25° C. to 80° C.; to provide an acid of formula I-3-i-5. An acid of formula I-3-i-5 is then acylated with a suitable chlorine source such as SOCl₂ or oxalyl chloride and the like; with a catalyst such as DMF and the like; in a suitable solvent such as DCM, dichloroethane, and the like; at temperatures ranging from 0° C. to 80° C.; to provide acyl chloride compound of formula I-3-i-6. Acyl chloride compound of formula I-3-i-6 is coupled with a commercially available or synthetically accessible amine source with a suitable organic base such as TEA, DIPEA and the like; in a suitable solvent such as DCM, THE and the like; at temperatures ranging from −10° C. to 20° C.; to provide an amide of formula I-3-i (R^{F4}=amide).

A compound of formula I-3-ii may be prepared according to the procedures shown in SCHEME 3.

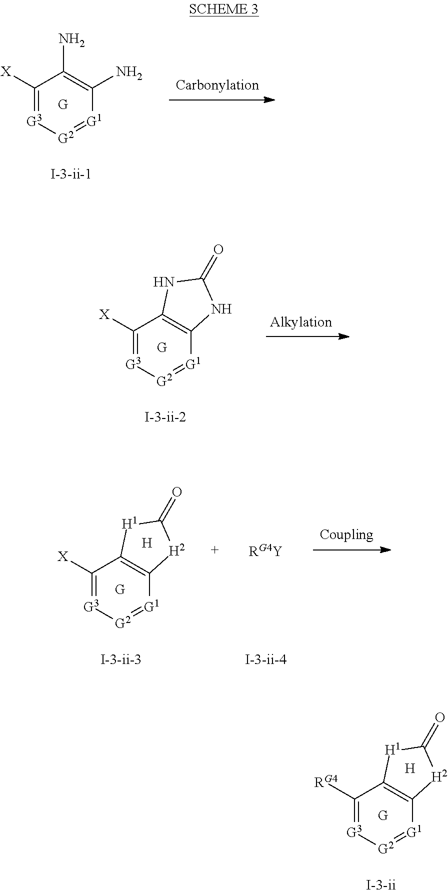

According to SCHEME 3, commercially available or synthetically accessible bis-aniline of formula I-3-ii-1 is reacted with a carbon monoxide insertion agent such as CDI, tri-phosgene or the like; with an organic base such as TEA or DIPEA or the like; in a suitable solvent such as DCM, DMF, and the like; at temperatures ranging from 0° C. to 60° C.; to provide urea compound of formula I-3-ii-2. A urea compound of formula I-3-ii-2 is alkylated with commercially available or synthetically accessible substituted alkyl halide with a suitable inorganic base such as NaH, t-BuOK and the like; in a suitable solvent such as DMF, THF, and the like; at temperatures ranging from 0° C. to 60° C., preferably 40° C.; to provide a substituted urea compound of formula I3-ii-3. A urea compound of formula I-3-ii-3 (X≡halide) is reacted with commercially available or synthetically accessible substituted alkyl or vinyl borate (Suzuki), zinc (Negishi) or magnesium (Kumada) agent I-3-ii-4; with a suitable catalysis such as Pd(Ph₃P)₄, PdCl₂(Ph₃P)₂ and the like; in an inorganic base such as Na₂CO₃, K₃PO₄ and the like; in a suitable solvent such as THF, dioxane and the like; at temperatures ranging from 0° C. to 100° C., preferably 40° C.; to provide a substituted compound of formula I3-ii.

A compound of formula I-3-iii may be prepared according to the procedures shown in SCHEME 4.

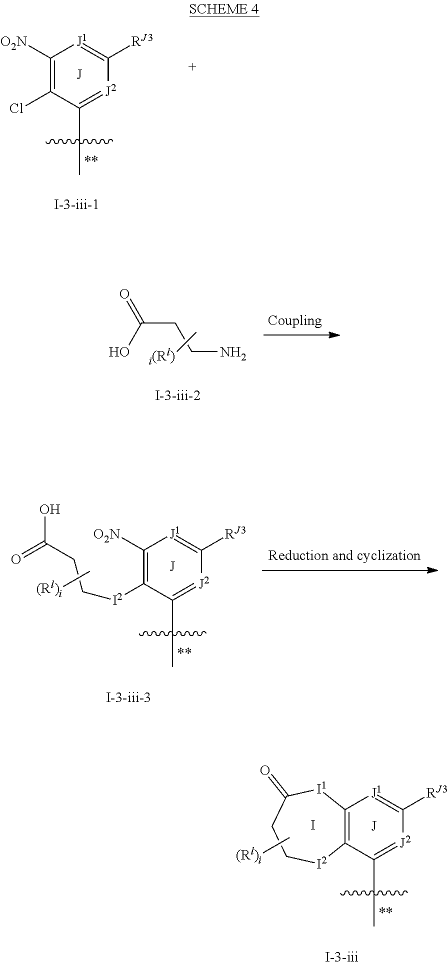

According to SCHEME 4, commercially available or synthetically accessible substituted aryl nitro compound of formula I-3-iii-1 is reacted with commercially available or synthetically accessible substituted amino acid of formula I-3-iii-2; with a suitable inorganic base such as K₂CO₃, Cs₂CO₃ and the like; in a suitable solvent such as DMF, DMSO and the like; at temperatures ranging from 50° C. to 120° C.; to provide coupled compound of formula I-3-iii-3. A nitro compound of formula I-3-iii-3 is reduced with a suitable reductant such as Fe, SnCl₂ and the like; in an acidic solvent such as HOAc, 1N HCl aqueous solution, and the like; at temperatures ranging from 80° C. to 120° C., preferably 100° C.; to provide a cyclized compound of formula I-3-iii.

A compound of formula I-3-iv may be prepared according to the procedures shown in SCHEME 5.

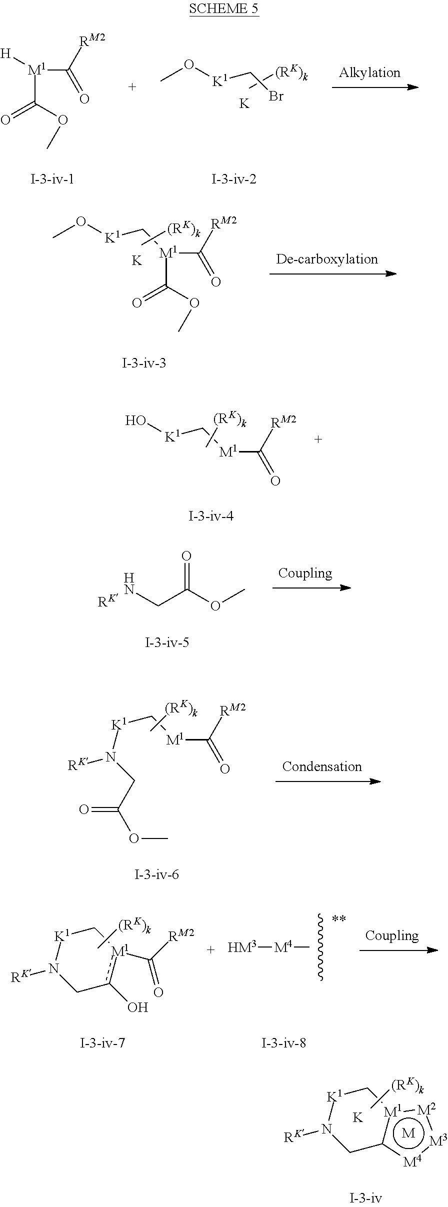

According to SCHEME 5, commercially available or synthetically accessible substituted keto ester compound of formula I-3-iv-1 is alkylated with commercially available or synthetically accessible substituted bromo-ester of formula I-3-iv-2; with a suitable inorganic base such as K₂CO₃, Cs₂CO₃ and the like; in a suitable solvent such as acetone, DMF, DMSO and the like; at temperatures ranging from 50° C. to 120° C.; to provide coupled compound of formula I-3-iv-3. A keto ester compound of formula I-3-iv-3 is de-carboxylated with a suitable acid such as HCl, H₂SO₄ and the like; in an acidic solvent such as HOAc, 1N HCl aqueous solution, and the like; at temperatures ranging from 40° C. to 100° C., preferably 60° C.; to provide a keto acid compound of formula I-3-iv-4. A keto acid compound of formula I-3-iv-4 is coupled with a commercially available or synthetically accessible substituted amino ester compound of formula I-3-iv-5; with a suitable coupling agent such as T3P, HATU and the like; in a suitable solvent such as DMF, DMSO and the like; at temperatures ranging from 0° C. to 80° C.; to provide coupled compound of formula I-3-iv-6. A coupled compound of formula I-3-iv-6 is cyclized under adol condensation condition; with a suitable base such as LDA, LiHMDS and the like; in a suitable solvent such as THF, dioxane and the like; at temperatures ranging from −78° C. to 0° C.; to provide cyclized keto-enol compound of formula I-3-iv-7. A keto-enol compound of formula I-3-iv-7 is reacted with commercially available or synthetically accessible substituted hydrazine compound of formula I-3-iv-8; in a suitable solvent such as MeOH, EtOH and the like; at temperatures ranging from 25° C. to 80° C.; to provide coupled compound of formula I-3-iv.

A compound of formula I-3-v may be prepared according to the procedures shown in SCHEME 6

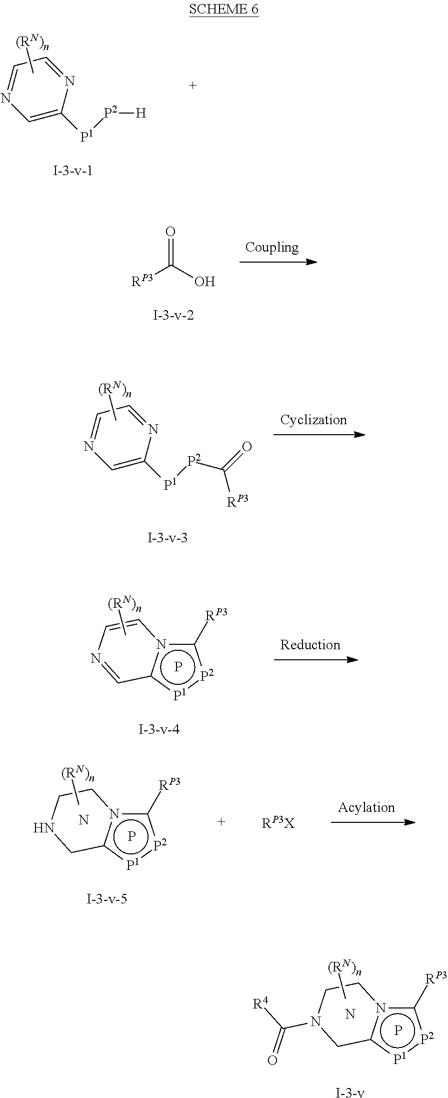

According to SCHEME 6, commercially available or synthetically accessible substituted amine compound of formula I-3-v-1 is coupled with commercially available or synthetically accessible substituted acid of formula I-3-v-2; with a suitable coupling agent such as HATU, T3P and the like; with a suitable organic base such as TEA, DIPEA and the like; in a suitable solvent such as DMF, DMSO and the like; at temperatures ranging from 0° C. to 80° C.; to provide coupled amide compound of formula I-3-v-3. An amide compound of formula I-3-v-3 is cyclized with a suitable reactive chloride source such as POCl₃, SOCl₂ and the like; in aprotic polar solvent such as DMF, and the like; at temperatures ranging from 60° C. to 100° C., preferably 80° C.; to provide a cyclized pyrazine compound of formula I-3-v-4. A cyclized pyrazine compound of formula I-3-v-4 is reduced with a suitable reducing agent such as up to 50 psi of H₂ gas, ammonium formate and the like; with a suitable catalysis such as 10% Pd/C, Pd(OH)₂/C and the like; in a suitable solvent such as EtOAc, MeOH and the like; at temperatures ranging from 25° C. to 80° C.; to provide reduced compound of formula I-3-v-5. A reduced compound of formula I-3-v-5 is acylated with a commercially available or synthetically accessible acylating agent of formula I-3-v-6; in a suitable organic base such as TEA, DIPEA and the like; in a suitable solvent such as DCM, dichloroethane and the like; at temperatures ranging from −10° C. to 25° C.; to provide acylated compound of formula I-3-v.

A compound of formula I-3-vi-a or I-3-vi-b may be prepared according to the procedures shown in SCHEME 7.

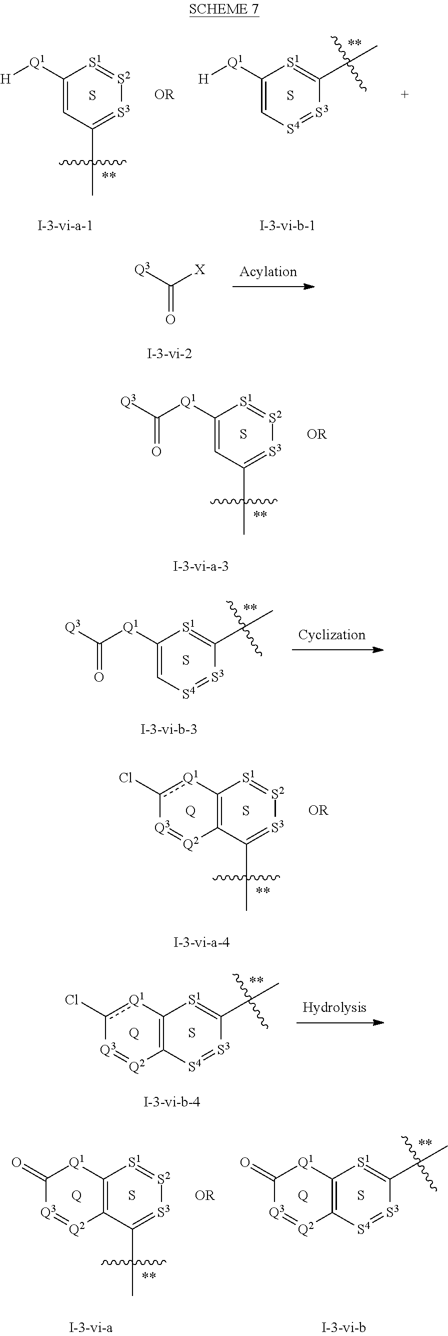

According to SCHEME 7, commercially available or synthetically accessible substituted aniline of formula I-3-vi-a-1 or I-3-vi-b-1 is reacted with commercially available or synthetically accessible substituted acyl halide (X≡Cl or Br) of formula I-3-vi-2; with a suitable organic base such as TEA, DIPEA and the like; in a suitable solvent such as DCM, dichloroethane and the like; at temperatures ranging from −10° C. to 50° C.; to provide coupled compound of formula I-3-vi-a-3 or I-3-vi-b-3. An acylated compound of formula I-3-vi-a-3 or I-3-vi-b-3 is reduced with a suitable chlorine source agent such as POCl₃, SOCl₂ and the like; in an aprotic polar solvent such as DMF, and the like; at temperatures ranging from 0° C. to 80° C.; to provide a cyclized compound of formula I-3-vi-a-4 or I-3-vi-b-4. An aryl chloride compound of formula I-3-vi-a-4 or I-3-vi-b-4 is hydrolized with a suitable base such as NaOH, KOH and the like; in a mixed solvent such as MeOH, and the like; with aqueous solution; at temperatures ranging from 0° C. to 60° C.; to provide a cyclized compound of formula I-3-vi-a or I-3-vi-b.

When L¹ is —C(═O)NH— optionally substituted with one or more R^{u}, a compound of formula I-2 may be prepared according to the procedures shown in SCHEME 8a.

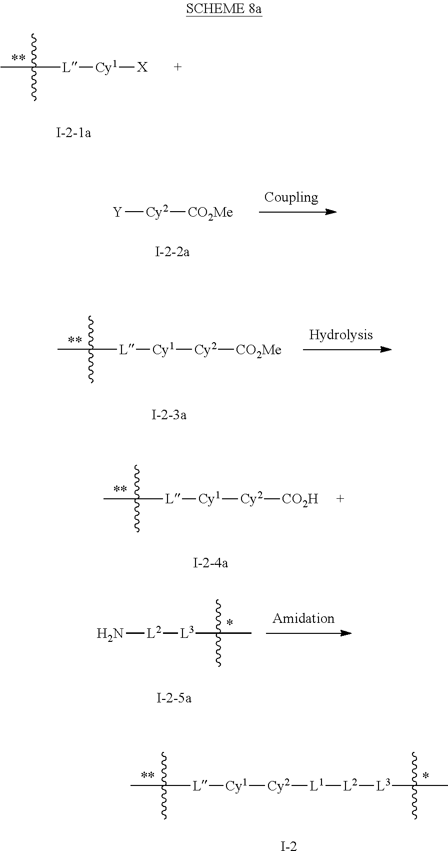

According to SCHEME 8a, commercially available or synthetically accessible substituted aryl halide (X≡Cl, Br, OTf, or the like) of formula I-2-1a is reacted with commercially available or synthetically accessible aryl metal agent (Y═B, Zn, Sn, Mg, or the like) of formula I-2-2a; with a suitable catalyst such as Pd(Ph₃P)₄, PdCl₂(dppf) or the like; with or without an inorganic base such as Na₂CO₃, K₃PO₄, or the like; in a suitable solvent such as 1,4-dioxane, DMF, or the like; at temperatures ranging from 60° C. to 100° C.; to provide coupled compound of formula I-2-3a. A coupled compound of formula I-2-3a is hydrolyzed with a suitable hydroxide source like lithium hydroxide or the like; in a suitable solvent like THE mixed with water, or the like; at temperatures ranging from 0° C. to 30° C.; to provide a carboxylate of formula I-2-4a. An acid of formula I-2-4a is reacted with commercially available or synthetically accessible substituted amine of formula I-2-5a; with a coupling agent such as HATU, T₃P, or the like; in an organic solvent such as DCM, DMF, or the like; at temperatures ranging from 0° C. to 60° C.; to provide a coupled amide compound of formula I-2.

Alternatively, when L¹ is 5- to 6-membered heteroaryl optionally substituted with one or more R^{u}, a compound of formula I-2 may be prepared according to the procedures shown in SCHEME 8b.

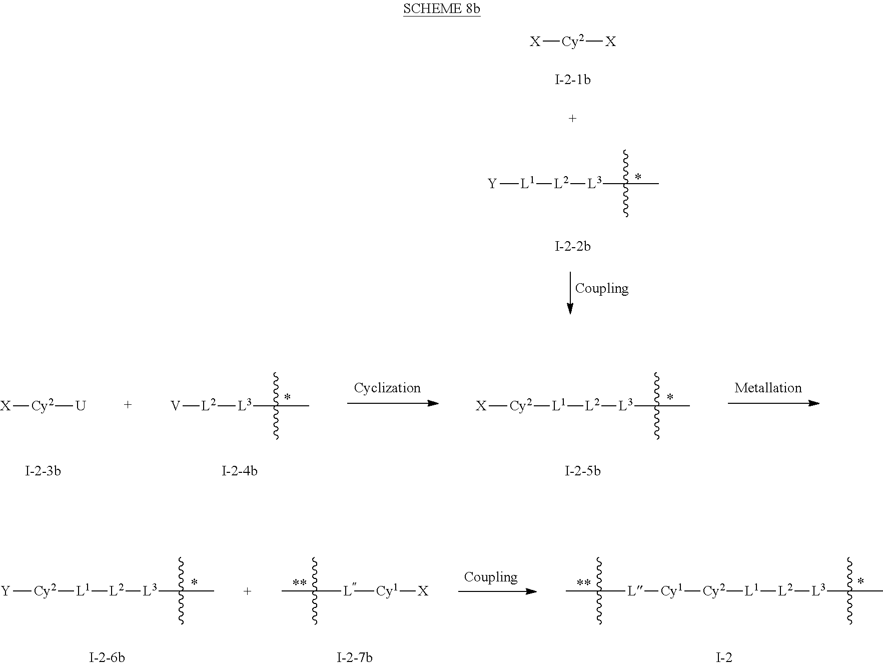

According to SCHEME 8b, commercially available or synthetically accessible substituted aryl halide (X═Cl, Br, OTf, or the like) of formula I-2-1b is reacted with commercially available or synthetically accessible aryl metal reagent (Y═B, Zn, Mg, Sn, or the like) of formula I-2-2b; with a suitable catalyst such as Pd(Ph₃P)₄, PdCl₂(dppf) or the like; with or without an inorganic base such as Na₂CO₃, K₃PO₄, or the like; in a suitable solvent such as 1,4-dioxane, DMF, or the like; at temperatures ranging from 60° C. to 100° C.; to provide coupled compound of formula I-2-5b. Alternatively, for certain instances of L¹, commercially available or synthetically accessible substituted aryl halide (X═Cl, Br, OTf, or the like) of formula I-2-3b (where U is an appropriate functional group to allow heterocycle synthesis) is reacted with commercially available or synthetically accessible alkyl reagent of formula I-2-4b (where V is a functional group that is complementary to U to allow for heterocycle synthesis); with reagents and under conditions known to those that are skilled in the art of organic synthesis; to provide a coupled compound of formula I-2-5b. An aryl halide of formula I-2-5b is converted to an aryl metal species of formula I-2-6b (Y═B, Zn, Mg, Sn, or the like); with an appropriate metal source such as B₂Pin₂, Bu₆Sn₂, ZnCl₂ or the like; with or without a suitable catalyst such as Pd(Ph₃P)₄, PdCl₂(dppf) or the like; with or without a lithiation reagent such as ^{t}BuLi or the like; in a suitable solvent such as 1,4-dioxane, THF, or the like; at temperatures ranging from 0° C. to 100° C.; to provide an aryl metal compound of formula I-2-6b. A nucleophile of formula I-2-6b is coupled with a synthetically accessible substituted aryl halide of formula I-2-7b (X═C₁, Br, OTf, or the like); with a suitable catalyst such as Pd(Ph₃P)₄, PdCl₂(dppf) or the like; with or without an inorganic base such as Na₂CO₃, K₃PO₄, or the like; in a suitable solvent such as 1,4-dioxane, DMF, or the like; at temperatures ranging from 60° C. to 100° C.; to provide a coupled compound of formula I-2.

A compound of formula I may be prepared according to the procedures shown in SCHEME 9.

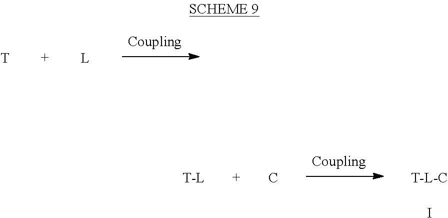

According to SCHEME 9, synthetically accessible substituted aryl halide as target ligand (Cl, Br or OTf) of formula T is reacted with synthetically accessible L (linker) (boronic ester, Zn or Mg halide) of formula L; with a suitable catalysis such as Pd(Ph₃P)₄, PdCl₂dppf and the like (Suzuki); or with a suitable catalysis such as Pd₂(dba)₃, Pd(OAc)₂ and the like (Buchwald) with or without an inorganic base such as Na₂CO₃, K₃PO₄, t-BuONa and the like; in a suitable solvent such as dioxane, DMF and the like; at temperatures ranging from 60° C. to 120° C.; to provide coupled compound of formula T-L. A coupled compound of formula T-L (target ligand and linker) is reacted with commercially available or synthetically accessible E3 ligand of formula C; with Suzuki coupling by catalysis of Pd(Ph₃P)₄, Pd₂(dba)₃ and the like; in an organic solvent such as dioxane, DMF, water, and the like; with an inorganic base such as Na₂CO₃, K₃PO₄ and the like; at temperatures ranging from 0° C. to 100° C.; to provide a coupled degrader compound of formula T-L-C (I).

Alternatively, a coupled compound of formula T-L (target ligand and linker) is reacted with commercially available or synthetically accessible E3 ligand of formula C; with Sonogashira coupling by catalysis of Pd(Ph₃P)₄, PdCl₂dppf and the like; or with a co-catalyst such as CuBr, CuI, and the like; in an organic solvent such as dioxane, DMF, and the like; with an organic base such as TEA, DIPEA and the like; at temperatures ranging from 0° C. to 100° C.; to provide a coupled degrader compound of formula T-L-C (I).

Those skilled in the art will recognize that the steps described in the schemes above may be inverted, depending on the specific compound. Those skilled in the art will also recognize that the steps from one scheme may be interspersed with those described in another scheme, as the reactivity of the specific compounds allow.

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-lnterscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka) (Pittsburgh, PA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Advanced Organic Chemistry: Reactions, Mechanisms and Structure”, 4th Ed., Wiley-Interscience, New York, 1992; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low resolution mass spectral data were measured using Agilent 1260-6125B LCMS system (with Diode Array Detector, and Agilent G6125BA Mass spectrometer) or using Waters Acquity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Acquity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Biological Assays

The biological activities of the compounds of the present disclosure can be assessed with methods and assays known in the art.

The binding potencies of the compounds to p300 may be determined using HTRF assay technology. HTRF signals may be measured by displacing the fluorescent probe, 5-(8-(7-acetyl-3-(tetrahydro-2H-pyran-4-yl)-5,6,7,8-tetrahydroimidazo[1,5-a]pyrazin-1-yl)isoquinolin-3-yl)-N-(2-(2-(2-(3-(3′,6′-dihydroxy-3-oxo-3H-spiro[isobenzofuran-1,9′-xanthen]-5-yl)thioureido)ethoxy)ethoxy)ethyl)picolinamide, from FLAG-tagged p300 with the tested compounds. Data analysis may be performed using appropriate software (e.g., TIBCO Spotfire) using a four-parameter dose response curve to determine the IC₅₀ of the tested compounds.

The binding potencies of the compounds to CBP may be determined using HTRF assay technology. HTRF signals may be measured by displacing the fluorescent probe, 5-(8-(7-acetyl-3-(tetrahydro-2H-pyran-4-yl)-5,6,7,8-tetrahydroimidazo[1,5-a]pyrazin-1-yl)isoquinolin-3-yl)-N-(2-(2-(2-(3-(3′,6′-dihydroxy-3-oxo-3H-spiro[isobenzofuran-1,9′-xanthen]-5-yl)thioureido)ethoxy)ethoxy)ethyl)picolinamide, from a FLAG-tagged (C-terminal) and His-tagged (N-terminal) CBP construct with the tested compounds. Data analysis may be performed using appropriate software (e.g., TIBCO Spotfire) using a four-parameter dose response curve to determine the IC₅₀ of the tested compounds.

The cellular degradation activities of the compounds against p300 may be measured by HiBit technology in certain cells (e.g., A549 P300 HiBit cells) with the tested compounds at certain initial concentrations (e.g., 0.000064, 0.00019, 0.00057, 0.0017, 0.0051, 0.015, 0.046, 0.14, 0.42, and 1.25 μM) for a certain period of time (e.g., 2 hours). The resulting protein concentration may be assessed using HiBit Lytic detection buffer. The resultant luminescence may be measured with an appropriate instrument (e.g., a PerkinElmer EnVision plate reader). Dose-response curves may be generated and analyzed using appropriate software (e.g., TIBCO Spotfire) to determine the DC₅₀ and Dₘₐₓ of the tested compounds.

The cellular degradation activities of the compounds against CBP may be measured by HiBit technology in certain cells (e.g., A549 CBP HiBit cells) with the tested compounds at certain initial concentrations (e.g., 0.000064, 0.00019, 0.00057, 0.0017, 0.0051, 0.015, 0.046, 0.14, 0.42, and 1.25 μM) for a certain period of time (e.g., 24 hours). The resulting protein concentration may be assessed using HiBit Lytic detection buffer. The resultant luminescence may be measured with an appropriate instrument (e.g., a PerkinElmer EnVision plate reader). Dose-response curves may be generated and analyzed using appropriate software (e.g., TIBCO Spotfire) to determine the DC₅₀ and Dₘₐₓ of the tested compounds.

The cellular degradation activities of the compounds against p300 may alternately be measured by in-cell Western blot technology in certain cells (e.g., H1299 cells) with the tested compounds at certain initial concentrations (e.g., 0.0005, 0.0015, 0.0046, 0.014, 0.041, 0.12, 0.37, 1.1, 3.3, and 10 μM) for a certain period of time (e.g., 16 hours). The resulting protein concentration may be assessed using an anti-p300 primary antibody (e.g., anti-human p300 clone D8Z4E) and an IRDye 800CW secondary antibody (e.g., IRDye 800CW goat anti-rabbit IgG). The resultant fluorescence may be measured with appropriate instrument (e.g., the LI-COR Odyssey CLx instrument). Dose-response curves may be generated and analyzed using appropriate software (e.g., TIBCO Spotfire) to determine the DC₅₀ and Dₘₐₓ of the tested compounds.

The cellular degradation activities of the compounds against CBP may alternately be measured by, e.g., in-cell Western blot technology in certain cells (e.g., H1299 cells) with the tested compounds at certain initial concentrations (e.g., 0.0005, 0.0015, 0.0046, 0.014, 0.041, 0.12, 0.37, 1.1, 3.3, and 10 μM) for a certain period of time (e.g., 16 hours). The resulting protein concentration may be assessed using an anti-CBP primary antibody (e.g., anti-human CBP clone D6C₅) and an IRDye 800CW secondary antibody (e.g., IRDye 800CW goat anti-rabbit IgG). The resultant fluorescence may be measured with appropriate instrument (e.g., the LI-COR Odyssey CLx instrument). Dose-response curves may be generated and analyzed using appropriate software (e.g., TIBCO Spotfire) to determine the DC₅₀ and Dₘₐₓ of the tested compounds.

The ability of the compounds to inhibit cellular proliferation may be assessed by, e.g., Cell Titer-Glo technology in wild-type, knock-out, and knock-out cell lines (e.g., H1299 wild-type, H1299 p300 knock-out, and H1299 CBP knock-out cell lines). The tested compounds may be assessed at certain initial concentrations (e.g., 0.0005, 0.0015, 0.0046, 0.014, 0.041, 0.12, 0.37, 1.1, 3.3, and 10 μM) for a certain period of time (e.g., 6 days). Cell growth may be assessed using the appropriate reagent and instrument (e.g., Cell Titer-Glo Luminescent Cell Viability reagent and a Perkin Elmer Envision instrument). Dose-response curves may be generated and analyzed using appropriate software (e.g., (TIBCO Spotfire) to determine the gIC₅₀ and the growth-death index (i.e. the percentage of remaining cell growth or cell death observed relative to untreated cells).

### Methods of Use

In certain aspects, the present disclosure provides methods of degrading a protein in a subject or biological sample comprising administering the compound disclosed herein to the subject or contacting the biological sample with the compound disclosed herein.

In certain aspects, the present disclosure provides uses of the compound disclosed herein in the manufacture of a medicament for degrading a protein in a subject or biological sample.

In certain aspects, the present disclosure provides compounds disclosed herein for use in degrading a protein in a subject or biological sample.

In certain aspects, the present disclosure provides methods of reducing a protein in a subject or biological sample comprising administering the compound disclosed herein to the subject or contacting the biological sample with the compound disclosed herein.

In certain aspects, the present disclosure provides uses of the compound disclosed herein in the manufacture of a medicament for reducing a protein in a subject or biological sample.

In certain aspects, the present disclosure provides compounds disclosed herein for use in reducing a protein in a subject or biological sample.

In certain embodiments, the protein is p300. In certain embodiments, the protein is CBP.

In certain aspects, the present disclosure provides methods for treating a disease or disorder.

In certain aspects, the present disclosure provides uses of the compounds disclosed herein in the manufacture of a medicament for treating a disease or disorder.

In certain aspects, the present disclosure provides compounds disclosed herein for treating a disease or disorder.

In certain embodiments, the disease or disorder is driven by gene activation such as cancer, inflammatory disorders, or autoimmune diseases.

In certain embodiments, the disease or disorder is selected from the group consisting of acoustic neuroma, acute leukemia, acute lymphocytic leukemia, acute myelocytic leukemia, acute T-cell leukemia, basal cell carcinoma, bile duct carcinoma, bladder cancer, brain choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen alfa receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

In certain embodiments, the disease or disorder is selected from the group consisting of Addison's disease, acute gout, ankylosing spondylitis, asthma, atherosclerosis, Behcet's disease, bullous skin diseases, chronic obstructive pulmonary disease, Crohn's disease, dermatitis, eczema, giant cell arteritis, fibrosis, glomerulonephritis, hepatic vascular occlusion, hepatitis, hypophysitis, immunodeficiency syndrome, inflammatory bowel disease, Kawasaki disease, lupus nephritis, multiple sclerosis, myocarditis, myositis, nephritis, organ transplant rejection, osteoarthritis, pancreatitis, pericarditis, Polyarteritis nodosa, pneumonitis, primary biliary cirrhosis, psoriasis, psoriatic arthritis, rheumatoid arthritis, scleritis, sclerosing cholangitis, sepsis, systemic lupus erythematosus, Takayasu's Arteritis, toxic shock, thyroiditis, type I diabetes, ulcerative colitis, uveitis, vitiligo, vasculitis, and Wegener's granulomatosis.

In certain embodiments, the disease or disorder is selected from the group consisting of prostate cancer, lung cancer, breast cancer, pancreatic cancer, colorectal cancer, and melanoma.

In certain embodiments, the subject is a mammal.

In certain embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.F. Eliel, Ed., Univ. of Notre Dame Press, Notre Dame, IN 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₅ alkyl”).

In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁-10 alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkylene” group may be unsubstituted or substituted or with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂4 alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄alkenyl groups as well as pentenyl (C₅), pentadienyl (C₅), hexenyl (C₆), and the like. Additional examples of alkenyl include heptenyl (C₇), octenyl (C₅), octatrienyl (C₅), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH═CH—). Exemplary substituted divalent alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₅), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂-10 alkynyl. In certain embodiments, the alkynyl group is substituted C₂₋₁₀ alkynyl.

“Alkynylene” as used herein, refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In certain embodiments, a heteroalkyl group is a saturated group having 1 to 9 carbon atoms and 1, 2, 3, or 4 heteroatoms (“heteroC₁₋₉ alkyl”). In certain embodiments, a heteroalkyl group is a saturated group having 1 to 8 carbon atoms and 1, 2, 3, or 4 heteroatoms (“heteroC₁₋₈ alkyl”). In certain embodiments, a heteroalkyl group is a saturated group having 1 to 7 carbon atoms and 1, 2, 3, or 4 heteroatoms (“heteroC₁₋₇ alkyl”). In certain embodiments, a heteroalkyl group is a group having 1 to 6 carbon atoms and 1, 2, or 3 heteroatoms (“heteroC₁₋₆ alkyl”). In certain embodiments, a heteroalkyl group is a saturated group having 1 to 5 carbon atoms and 1 or 2 heteroatoms (“heteroC₁₋₅ alkyl”). In certain embodiments, a heteroalkyl group is a saturated group having 1 to 4 carbon atoms and/or 2 heteroatoms (“heteroC₁₋₄ alkyl”). In certain embodiments, a heteroalkyl group is a saturated group having 1 to 3 carbon atoms and 1 heteroatom (“heteroC₁₋₃ alkyl”). In certain embodiments, a heteroalkyl group is a saturated group having 1 to 2 carbon atoms and 1 heteroatom (“heteroC₁₋₂ alkyl”). In certain embodiments, a heteroalkyl group is a saturated group having 1 carbon atom and 1 heteroatom (“heteroC₁ alkyl”). In certain embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In certain embodiments, a heteroalkenyl group has 2 to 9 carbon atoms at least one double bond, and 1, 2, 3, or 4 heteroatoms (“heteroC₂₋₉ alkenyl”). In certain embodiments, a heteroalkenyl group has 2 to 8 carbon atoms, at least one double bond, and 1, 2, 3, or 4 heteroatoms (“heteroC₂₋₈ alkenyl”). In certain embodiments, a heteroalkenyl group has 2 to 7 carbon atoms, at least one double bond, and 1, 2, 3, or 4 heteroatoms (“heteroC₂₋₇ alkenyl”). In certain embodiments, a heteroalkenyl group has 2 to 6 carbon atoms, at least one double bond, and 1, 2, or 3 heteroatoms (“heteroC₂₋₆ alkenyl”). In certain embodiments, a heteroalkenyl group has 2 to 5 carbon atoms, at least one double bond, and 1 or 2 heteroatoms (“heteroC₂₋₅ alkenyl”). In certain embodiments, a heteroalkenyl group has 2 to 4 carbon atoms, at least one double bond, and lor 2 heteroatoms (“heteroC₂₋₄ alkenyl”). In certain embodiments, a heteroalkenyl group has 2 to 3 carbon atoms, at least one double bond, and 1 heteroatom (“heteroC₂₋₃ alkenyl”). In certain embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In certain embodiments, a heteroalkynyl group has 2 to 9 carbon atoms, at least one triple bond, and 1, 2, 3, or 4 heteroatoms (“heteroC₂₋₉ alkynyl”). In certain embodiments, a heteroalkynyl group has 2 to 8 carbon atoms, at least one triple bond, and 1, 2, 3, or 4 heteroatoms (“heteroC₂₋₈ alkynyl”). In certain embodiments, a heteroalkynyl group has 2 to 7 carbon atoms, at least one triple bond, and 1, 2, 3, or 4 heteroatoms (“heteroC₂₋₇ alkynyl”). In certain embodiments, a heteroalkynyl group has 2 to 6 carbon atoms, at least one triple bond, and 1, 2, or 3 heteroatoms (“heteroC₂₋₆ alkynyl”). In certain embodiments, a heteroalkynyl group has 2 to 5 carbon atoms, at least one triple bond, and 1 or 2 heteroatoms (“heteroC₂₋₅ alkynyl”). In certain embodiments, a heteroalkynyl group has 2 to 4 carbon atoms, at least one triple bond, and lor 2 heteroatoms (“heteroC₂₋₄ alkynyl”). In certain embodiments, a heteroalkynyl group has 2 to 3 carbon atoms, at least one triple bond, and 1 heteroatom (“heteroC₂₋₃ alkynyl”). In certain embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 7L electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Arylene” as used herein, refers to an aryl group wherein two hydrogens are removed to provide a divalent radical. When a range or number of carbons is provided for a particular “arylene” group, it is understood that the range or number refers to the range or number of carbons in the aryl group. An “arylene” group may be substituted or unsubstituted with one or more substituents as described herein.

“Heteroaryl” refers to a radical of a 5- to 14-membered monocyclic or polycyclic 4n+2 aromatic ring system (e.g., having 6, 10, or 14 7L electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroarylene” as used herein, refers to a heteroaryl group wherein two hydrogens are removed to provide a divalent radical. When a range or number of ring members is provided for a particular “heteroarylene” group, it is understood that the range or number refers to the number of ring members in the heteroaryl group. A “heteroarylene” group may be substituted or unsubstituted with one or more substituents as described herein.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₅). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

As the foregoing examples illustrate, in certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃₋12 carbocyclyl.

“Fused carbocyclyl” or “fused carbocycle” refers to ring systems wherein the carbocyclyl group, as defined above, is fused with, i.e., share two common atoms (as such, share one common bond), one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or or “bridged carbocycle” refers to ring systems wherein the carbocyclyl group, as defined above, form bridged structure with, i.e., share more than two atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the carbocyclyl rings in which the bridged structure is embedded. When substitution is indicated, unless otherwise specified, substitution can occur on any of the carbocyclyl rings in which the bridged structure is embedded.

“Carbocyclylene” as used herein, refers to a carbocyclyl group wherein two hydrogens are removed to provide a divalent radical. The divalent radical may be present on different atoms or the same atom of the carbocyclylene group. When a range or number of carbons is provided for a particular “carbocyclyl” group, it is understood that the range or number refers to the range or number of carbons in the carbocyclyl group. A “carbocyclyl” group may be substituted or unsubstituted with one or more substituents as described herein.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

As the foregoing examples illustrate, in certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share two common atoms (as such, share one common bond) with, one or more heterocyclyl or carbocyclyl groups, as defined above, wherein the point of attachment is on any of the fused rings. In such instances, the number of ring members designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than two atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the heterocyclyl or carbocyclyl rings in which the bridged structure is embedded.

“Heterocyclylene” as used herein, refers to a heterocyclyl group wherein two hydrogens are removed to provide a divalent radical. The divalent radical may be present on different atoms or the same atom of the heterocyclylene group. When a range or number of ring members is provided for a particular “heterocyclylene” group, it is understood that the range or number refers to the number of ring members in the heterocyclylene group. A “heterocyclylene” group may be substituted or unsubstituted with one or more substituents as described herein.

“Alkoxy” as used herein, refers to the group —OR, wherein R is alkyl as defined herein. C₁₋₆ alkoxy refers to the group —OR, wherein each R is C₁₋₆ alkyl, as defined herein. Exemplary C₁₋₆ alkyl is set forth above.

“Alkylamino” as used herein, refers to the group —NHR or —NR₂, wherein each R is independently alkyl, as defined herein. C₁₋₆ alkylamino refers to the group —NHR or —NR₂, wherein each R is independently C₁₋₆ alkyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro (Cl), bromo (Br), and iodo (I). In certain embodiments, the halo group is either fluoro or chloro.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), P-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g, benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-t-butyl ester, silyl esters, and orthoesters) and oxazoline.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

A “subject” to which administration is contemplated includes, but is not limited to, humans (i.e., a male or female of any age group, e.g., a pediatric subject (e.g., infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range. In certain embodiments, the number or numerical range vary by 1%, 2%, 3%, 4%, or 5% of the stated number or numerical range. In certain embodiments, the number or numerical range vary by 1%, 2%, or 3% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### Exemplary Embodiments

Exemplary Embodiment No. 1. A compound of Formula I:

T-L-C(I),

or a pharmaceutically acceptable salt, solvate, or stereoisomer thereof, wherein: C is of Formula I-1

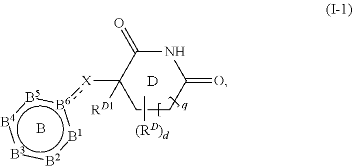

wherein:
[symbol] denotes a single bond or a double bond;
B¹ is CRB, N, NR^{B1′}, O, or S;
B² is CR^{B2}, N, NR^{B2′}, O, or S;
B³ is absent, CR^{B3}, or N;
B⁴ is CR^{B4}, N, NR^{B4′}, O, or S;
B⁵ is CRBS, N, NR^{B5′}, O, or S;
B⁶ is C or N;
R^{B1}, R^{B2}, R^{B3}, R^{B4}, and R^{B5} are independently

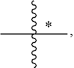

hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}, and one of R^{B1}, R^{B2}, R^{B3}, R^{B4}, and R^{B5} is

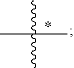

or
R^{B4} and R^{B5}, together with the carbon atoms to which they are bonded, form 3- to 12-membered heterocyclyl optionally substituted with one or more R^{u};
R^{B1′}, R^{B2′}, R^{B4′}, and R^{B5′} are independently hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
X is absent, CRxl, C(R^{X1})₂, NR^{X2} or O, as valency permits;
each R^{X1} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
two R^{X1} together form an oxo; or
two R^{X1}, together with the carbon atom to which they are bonded, form C₃₋₄ carbocyclyl or 3- to 4-membered heterocyclyl, wherein the carbocyclyl or heterocyclyl is optionally substituted with one or more R;
R^{X2} is hydrogen, C₁₋₃ alkyl, C₃₋₄ carbocyclyl, or 3- to 4-membered heterocyclyl, wherein the alkyl, carbocyclyl, or heterocyclyl is optionally substituted with one or more R^{u}; or
R^{X1} and R^{B5}, R^{X1} and R^{B5′}, R^{X2} and R^{B5}, or R^{X2} and R^{B5′}, together with the atoms to which they are bonded, form Ring C, wherein Ring C is optionally substituted C₆ aryl, optionally substituted 5- to 6-membered heteroaryl, optionally substituted 5- to 6-membered heterocycle, or optionally substituted C₅₋₆ carbocycle;
R^{D1} is hydrogen, deuterium, halogen, C₁₋₆ alkyl, or C₁₋₆ haloalkyl; or
R^{D1} and one R^{D}, together with the carbon atoms to which they are bonded, form C₃₋₄ carbocycle or 3- to 4-membered heterocycle, wherein the carbocycle or heterocycle is optionally substituted with one or more R^{u};
each R^{D} is independently oxo, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; or
two R^{D}, together with the carbon atom(s) to which they are bonded, form C₃₋₅ carbocyclyl or 3- or 5-membered heterocyclyl, wherein the carbocyclyl or heterocyclyl is optionally substituted with one or more R^{u};
d is an integer from 0 to 6, as valency permits; and
q is an integer from 0 to 2,
wherein when X is NR^{X2} or O, and L″ is absent, then i) R^{X2} and R^{B5}, together with the atoms to which they are bonded, form Ring C; ii) B³ is absent; or iii) R^{D1} and one R^{D}, together with the carbon atoms to which they are bonded, form C₃₋₄ carbocycle or 3- to 4-membered heterocycle, wherein the carbocycle or heterocycle is optionally substituted with one or more R^{u},
T is
i) of Formula I-3-i

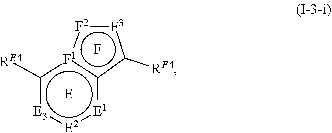

wherein:
E¹ is

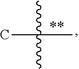

### CR^{E1}, or N;

E² is

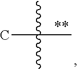

### CR^{E2}, or N;

E³ is

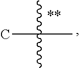

### CR^{E3}, or N;

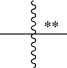

denotes attachment to L;
R^{E1}, R^{E2}, R^{E3}, and R^{E4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
F¹ is C or N;
F² is NR^{F2′} or CR^{F2};
R^{F2′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R^{F2} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
F³ is CR^{F3}, N, or NR^{F3′};
R^{F3} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—NR^{b}C(═O)R^{a}, —N^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R;
R^{F3′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R^{F4} is —C(═O)NR^{3a}R^{3b} or 5- to 6-membered heteroaryl optionally substituted with one or more R^{u}; and
R^{3a} and R^{3b} are independently hydrogen, C₁₋₆ alkyl, C₂₋₆ alkenyl, C₂₋₆ alkynyl, C₃₋₁₂ carbocyclyl, 3to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R; or
R^{3a} and R^{3b}, together with the nitrogen atom to which they are attached, form 3- to 12-membered heterocycle or 5- to 10-membered heteroaryl, wherein the heterocycle or heteroaryl is optionally substituted with one or more R^{u},
ii) of Formula I-3-ii

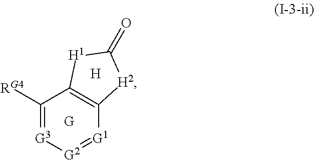

wherein:
G¹ is

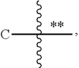

### N, or CR;

G² is

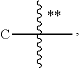

### N, or CR^{G2};

G³ is

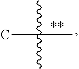

### N, or CR^{G3};

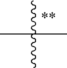

denotes attachment to L;
R^{G1}, R^{G2}, R^{G3}, and R^{G4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
H¹ is C(R^{H1})₂, NR^{H1′} or O;
H² is C(R^{H2})₂, NR^{H2′} or O;
each occurrence of R^{H11} and R^{H2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl wherein the alkyl, alkoxy, alkylamino, alkenyl, alkynyl, carbocyclyl, heterocyclyl, aryl, or heteroaryl is optionally substituted with one or more R^{u}; or
two R^{H11} or two R^{H2}, together with the carbon atom to which they are attached, form C₃₋₆ carbocycle or 3- to 6-membered heterocycle, wherein the carbocycle or heterocycle is optionally substituted with one or more R^{u}; and
R^{H1′} and R^{H2′} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}
iii) of Formula I-3-iii

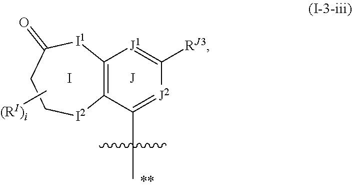

wherein:

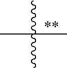

denotes attachment to L;
I¹ is C(R)₂, NR^{I1′} or O;
I² is C(R^{I2})₂, NR^{I2′} or O;
each occurrence of R¹ and R^{I2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{I1} or two R^{I2}, together with the carbon atom to which they are attached, form C₃₋₆ carbocycle or 3- to 6-membered heterocycle, wherein the carbocycle or heterocycle is optionally substituted with one or more R^{u}
R^{I1′} and R^{I2′} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
each R^{I} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
i is an integer selected from 0 to 4;
J¹ is N or CR^{J1};
J² is N or CR^{J2}; and
R^{J}i, R^{J2}, and R^{J3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}
iv) of Formula I-3-iv

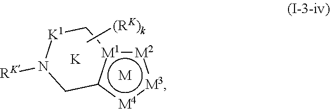

wherein:
M¹ is N or C;
M² is

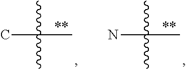

### N, or CR^{M2}.

M³ is

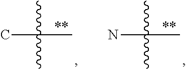

### T, or CR^{M3};

M⁴ is

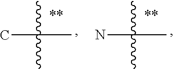

### N, or CR^{M4}.

wherein

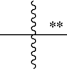

denotes attachment to L; and at least one of M¹, M², M³, and M⁴ is N or;

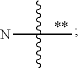 [SMILES: *N]

R^{M2}, R^{M3}, and R^{M4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}
R^{K′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}
K¹ is —C(═O)— or —C(RK1)₂—;
each R^{K1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
each R^{K} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
k is an integer selected from 0 to 4;
v) of Formula I-3-v

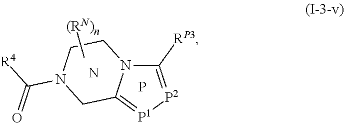

wherein:
P¹ is

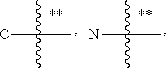

### N, or CR^{P1}

P² is

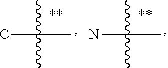

### N or CR^{P2}.

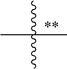

denotes attachment to L;
each R^{N} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
n is an integer selected from 0 to 6;
R^{P1}, R^{P2}, and R^{P3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
R⁴ is —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u},
or
vi) of Formula I-3-vi

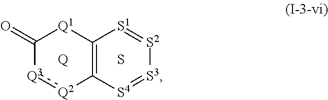

wherein:
Q¹ is NR^{Q1′}, C(R^{Q1})₂, or O;
R^{Q1′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}
each R^{Q1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{Q1}, together with the carbon atom to which they are attached, form C₃₋₆ carbocycle or 3- to 6-membered heterocycle, wherein the carbocycle or heterocycle is optionally substituted with one or more R^{u}
when - is a double bond, then Q² is N or CR^{Q2}; and Q³ is N or CR^{Q3};
when - is a single bond, then Q² is NR^{Q2′}, C(R^{Q2})₂, or O; Q³ is NR^{Q3′}, C(R^{Q3})₂, or O; R^{Q2′} and R^{Q3′} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
each occurrence of R^{Q2} and R^{Q3} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{Q2} or two R^{Q3}, together with the carbon atom to which they are attached, form C₃₋₆ carbocycle or 3- to 6-membered heterocycle, wherein the carbocycle or heterocycle is optionally substituted with one or more R^{u};
S¹ is CR^{S1} or N;
S² is

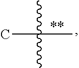

### CR^{S2}, or N;

S³ is

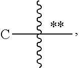

### CR^{S3}, or N;

S⁴ is

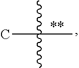

### CR^{S4}, or N;

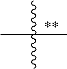

denotes attachment to L; and
R^{S1}, R^{S2}, R^{S3}, and R^{S4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
L is of Formula I-2

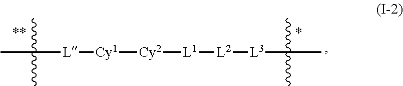

wherein:
** denotes attachment to T and * denotes attachment to C;
L″ is absent; or
L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u};
wherein:
i) when L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u}, then Cy¹ is optionally substituted C₆ arylene or 5- to 6-membered heteroarylene;
ii) when L″ is absent, then
   Cy¹ is

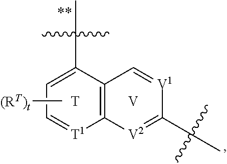

wherein:
** denotes attachment to T;
T^{t} is CR^{T1} or N;
V¹ is CRy¹ or N;
V² is CR^{V2} or N;
R^{T1}, R^{V1}, and R^{V2} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
each R^{T} is independently halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
t is an integer selected from 0 to 6, as valency permits,
wherein R^{T} may be present on either Ring T or Ring V;
or
Cy¹ is

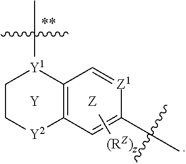

wherein:
Y¹ is N or CR^{Y1};
** denotes attachment to T;
R^{Y1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
Y² is C(R^{Y2})₂, O, or N(R^{Y2′});
each R^{Y2} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
R^{Y2′} is hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
Z¹ is CR^{Z1} or N;
R^{Z1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
each R^{Z} is independently oxo, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
z is an integer selected from 0 to 6, as valency permits,
wherein R^{Z} may be present on either Ring Y or Ring Z;
Cy² is 5- to 6-membered heteroarylene optionally substituted with one or more R^{u}
L¹ is —C(═O)NH— or 5- to 6-membered heteroaryl optionally substituted with one or more R^{u};
L² is —C(R^{L2})₂—, wherein
each R^{L2} is independently hydrogen, C₁₋₃ alkyl, or C₁₋₃ haloalkyl; or
two geminal R^{L2}, together with the carbon atom to which they are bonded, form C₃₋₅ carbocyclyl or 3- to 5-membered heterocyclyl, wherein the carbocyclyl or heterocyclyl is optionally substituted with one or more R^{u};
and
L³ is E-ethenylene, ethynylene, C₆ arylene, or 5- to 6-membered heteroarylene, wherein the E-ethenylene, arylene, or heteroarylene is optionally substituted with one or more R^{u}
wherein:
each R^{u}; is independently oxo, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl;
each R^{a} is independently C₁₋₆ alkyl, C₃₋₆ carbocyclyl, or 3- to 6-membered heterocyclyl;
each R^{b} is independently hydrogen, C₁₋₆ alkyl, C₃₋₆ carbocyclyl, or 3- to 6-membered heterocyclyl; and
each R^{c} and R^{d} is independently hydrogen, C₁₋₆ alkyl, C₃₋₆ carbocyclyl, or 3- to 6-membered heterocyclyl; or R^{c} and R^{d}, together with the nitrogen atom to which they are attached, form 3- to 6-membered heterocyclyl,
wherein each occurrence of R^{a}, R^{b}, R^{c}, and R^{d} is independently and optionally substituted with one or more R^{Z}; and
each R^{Z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl.

Exemplary Embodiment No. 2. The compound of Exemplary Embodiment No. 1, wherein RXl and R^{B5}, Rxl and R^{B5′}, R^{X2} and R^{B5}, or R^{X2} and R^{B5′}, together with the atoms to which they are bonded, do not form Ring C, and R^{B2} or R^{B3} is

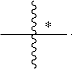

Exemplary Embodiment No. 3. The compound of Exemplary Embodiment No. 2, wherein C is of Formula I-1-i or I-1-ii

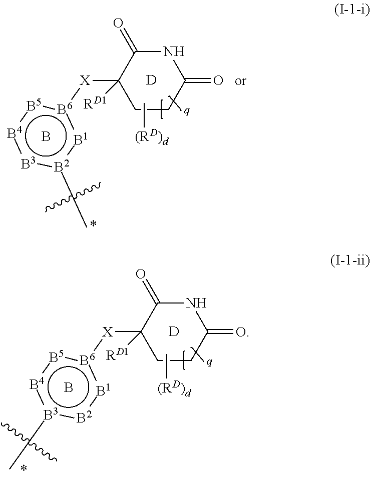

Exemplary Embodiment No. 4. The compound of Exemplary Embodiment No. 3, wherein C is of Formula I-1-i

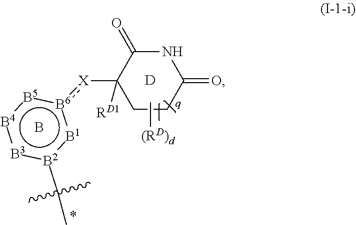

wherein:
none of B¹, B³, B⁴, and B⁵ is N, or one of B¹ and B⁵ is N, wherein R^{B1}, R^{B3}, R^{B4}, and R^{B5}, R^{B1}, R^{B3} and R^{B4}, or R^{B3}, R^{B4}, and R^{B5} are independently hydrogen, halogen, —CN, —OH, C₁₋₆ alkyl, C₁₋₆ alkoxy, or C₁₋₆ alkylamino.

Exemplary Embodiment No. 5. The compound of Exemplary Embodiment No. 1, wherein RXl and RBs, Rx1 and R^{B5′}, R^{X2} and R^{B5}, or R^{X2} and R^{B5′}, together with the atoms to which they are bonded, form Ring C, wherein Ring C is optionally substituted C₆ aryl or optionally substituted 5-membered heteroaryl.

Exemplary Embodiment No. 6. The compound of Exemplary Embodiment No. 5, wherein C is of Formula I-1-iii

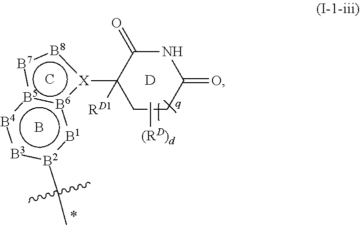

wherein:
B^{5a} is C or N;
B⁷ is CR^{B7}, N, NR^{B7′}, O, or S;
B⁸ is CR^{B8}, N, NR^{B8′}, O, or S;
R^{B7} and R^{B8} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; and
R^{B7′} and R^{B8′} are independently hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u},
wherein at least one of B^{5a}, B⁶, B⁷, B⁸, and X is N, NR^{B7′} NR^{B8}′, O, or S.

Exemplary Embodiment No. 7. The compound of Exemplary Embodiment No. 6, wherein C is of Formula I-1-iii-1, I-1-iii-2, I-1-iii-3, or I-1-iii-4

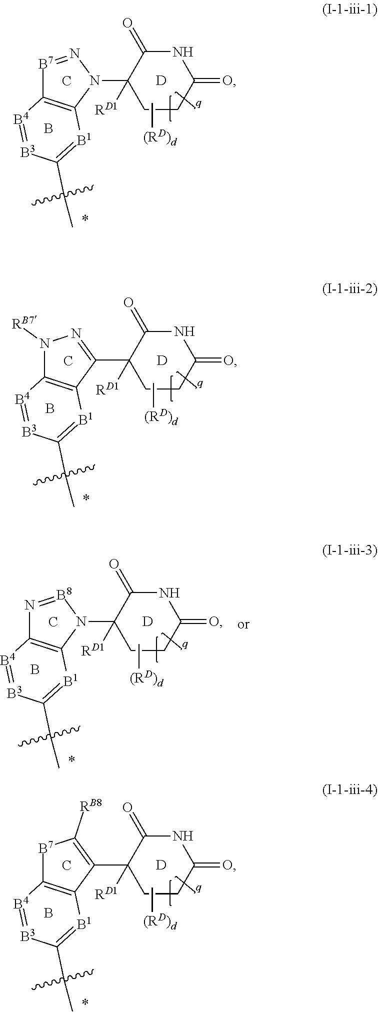

wherein when C is of Formula I-1-iii-4, B⁷ is O or S.

Exemplary Embodiment No. 8. The compound of Exemplary Embodiment No. 7, wherein C is of Formula I-1-iii-1, and B⁷ is CR^{B7}, and R^{B7} is hydrogen or C₁₋₆ alkyl; C is of Formula I-1-iii-2, and R^{B7′} is hydrogen or C₁₋₆ alkyl; or C is of Formula I-1-iii-3, and B⁸ is CR^{B8}, and R^{B8} is hydrogen or C₁₋₆ alkyl.

Exemplary Embodiment No. 9. The compound of Exemplary Embodiment No. 7 or 8, wherein none of B¹, B³, and B⁴ is N and each of R^{B1}, R^{B3}, and R^{B4} is hydrogen.

Exemplary Embodiment No. 10. The compound of Exemplary Embodiment No. 5, wherein C is of Formula I-1-iv

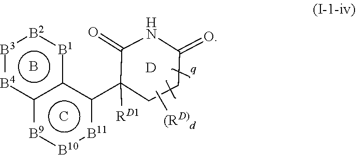

wherein:
B⁹ is CR^{B9} or N;
B¹⁰ is CR^{B10} or N;
B¹¹ is CR^{B11} or N; and
R^{B9}, R^{B10}, and R^{B11} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}.

Exemplary Embodiment No. 11. The compound of Exemplary Embodiment No. 10, wherein C is of Formula I-1-iv-1

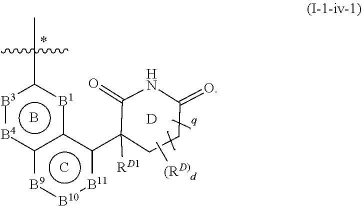

Exemplary Embodiment No. 12. The compound of Exemplary Embodiment No. 11, wherein none of B⁹, B¹⁰, and B¹¹ is N, and each of R^{B9}, R^{B10}, and R^{B11} is hydrogen.

Exemplary Embodiment No. 13. The compound of any one of Exemplary Embodiment Nos. 12, wherein none of B¹, B³, and B⁴ is N and each of R^{B1}, R^{B3}, and R^{B4} is hydrogen.

Exemplary Embodiment No. 14. The compound of any one of Exemplary Embodiment Nos. 1-13, wherein R^{D}1 is hydrogen.

Exemplary Embodiment No. 15. The compound of any one of Exemplary Embodiment Nos. 1-14, wherein d is 0.

Exemplary Embodiment No. 16. The compound of any one of Exemplary Embodiment Nos. 1-15, wherein q is 1.

Exemplary Embodiment No. 17. The compound of any one of Exemplary Embodiment Nos. 1-16, wherein T is of Formula I-3-i

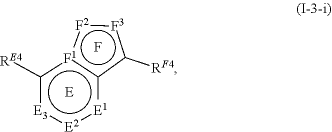

Exemplary Embodiment No. 18. The compound of Exemplary Embodiment No. 17, wherein T is of Formula I-3-i-a

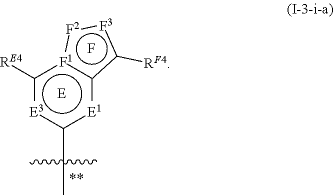

Exemplary Embodiment No. 19. The compound of Exemplary Embodiment No. 17 or 18, wherein F² is NR^{F2}′

Exemplary Embodiment No. 20. The compound of any one of Exemplary Embodiment Nos. 17-19, wherein R^{F4} is —C(═O)NR^{3a}R^{3b}.

Exemplary Embodiment No. 21. The compound of any one of Exemplary Embodiment Nos. 17-20, wherein F¹ is C.

Exemplary Embodiment No. 22. The compound of any one of Exemplary Embodiment Nos. 17-20, wherein F¹ is N.

Exemplary Embodiment No. 23. The compound of Exemplary Embodiment No. 17, wherein T is of Formula I-3-i-b

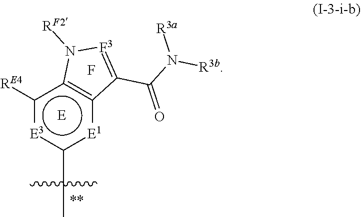

Exemplary Embodiment No. 24. The compound of any one of Exemplary Embodiment Nos. 17-23, wherein R^{F2′} is hydrogen or C₁₋₆ alkyl optionally substituted with one or more R^{u}.

Exemplary Embodiment No. 25. The compound of any one of Exemplary Embodiment Nos. 17-24, wherein R^{3a} and R^{3b} are independently hydrogen or C₁₋₆ alkyl optionally substituted with one or more R.

Exemplary Embodiment No. 26. The compound of any one of Exemplary Embodiment Nos. 17-25, wherein E¹ is CR^{E1}, and E³ is CR^{E3}, wherein R^{E}i and R^{E2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

Exemplary Embodiment No. 27. The compound of any one of Exemplary Embodiment Nos. 17-25, wherein one of E¹ and E³ is N, wherein R^{E1} or R^{E3} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

Exemplary Embodiment No. 28. The compound of any one of Exemplary Embodiment Nos. 17-27, wherein R^{E}i and R^{E3} are independently or R^{E}i or R^{E2} is hydrogen, halogen, or C₁₋₆ alkyl optionally substituted with one or more R.

Exemplary Embodiment No. 29. The compound of any one of Exemplary Embodiment Nos. 17-28, wherein R^{E4} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

Exemplary Embodiment No. 30. The compound of any one of Exemplary Embodiment Nos. 17-28, wherein R^{E4} is hydrogen, halogen, —CN, C₁₋₆ alkyl, C₁₋₆ alkoxy, C₁₋₆ alkylamino, C₃₋₁₂ carbocyclyl, 3- to 12-membered heterocyclyl, or —S(═O)₂R^{a}, wherein the alkyl, alkoxy, alkylamino, carbocyclyl, or heterocyclyl is optionally substituted with one or more R^{u}.

Exemplary Embodiment No. 31. The compound of any one of Exemplary Embodiment Nos. 17-30, wherein F³ is CR^{F3}, wherein R^{F3} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

Exemplary Embodiment No. 32. The compound of Exemplary Embodiment No. 31, wherein R^{F3} is hydrogen.

Exemplary Embodiment No. 33. The compound of any one of Exemplary Embodiment Nos. 17-32, wherein F³ is N.

Exemplary Embodiment No. 34. The compound of any one of Exemplary Embodiment Nos. 1-16, wherein T is of Formula I-3-ii

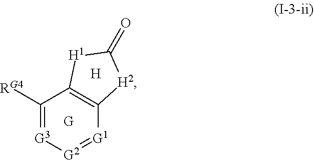

Exemplary Embodiment No. 35. The compound of Exemplary Embodiment No. 34, wherein T is of Formula I-3-ii-a

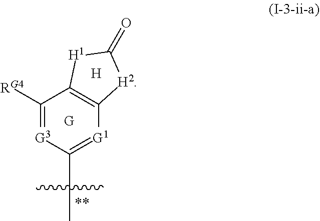

Exemplary Embodiment No. 36. The compound of Exemplary Embodiment No. 34 or 35, wherein G¹ is CR^{G1}, and G³ is CR^{G3}, wherein R^{G}l and R^{G3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

Exemplary Embodiment No. 37. The compound of Exemplary Embodiment No. 36, wherein each of R^{G}l and R^{G3} is hydrogen.

Exemplary Embodiment No. 38. The compound of any one of Exemplary Embodiment Nos. 34-37, wherein R^{G4} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

Exemplary Embodiment No. 39. The compound of Exemplary Embodiment No. 38, wherein R^{G4} is hydrogen, C₁₋₆ alkyl, C₁₋₆ alkoxy, C₁₋₆ alkylamino, C₂₋₆ alkenyl, or 3- to 12-membered heterocyclyl, wherein the alkyl, alkoxy, alkylamino, alkenyl, or heterocyclyl is optionally substituted with one or more R.

Exemplary Embodiment No. 40. The compound of any one of Exemplary Embodiment Nos. 34-39, wherein H¹ is NR^{H11′}, and H² is NR^{H2′}, wherein R^{H11′} and R^{H2′} are independently C₁₋₆ alkyl.

Exemplary Embodiment No. 41. The compound of any one of Exemplary Embodiment Nos. 1-16, wherein T is of Formula I-3-iii

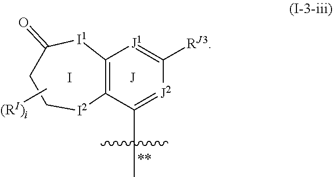

Exemplary Embodiment No. 42. The compound of Exemplary Embodiment No. 41, wherein I is NR^{I1}; and I² is NR^{I2′}, wherein R^{I1} and R^{I2′} are independently hydrogen or C₁₋₆ alkyl.

Exemplary Embodiment No. 43. The compound of Exemplary Embodiment No. 41 or 42, wherein at least one R¹ is C₁₋₆ alkyl.

Exemplary Embodiment No. 44. The compound of Exemplary Embodiment No. 41, wherein T is of Formula I-3-iii-a

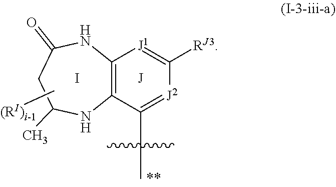

Exemplary Embodiment No. 45. The compound of any one of Exemplary Embodiment Nos. 41-44, wherein each R¹ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

Exemplary Embodiment No. 46. The compound of any one of Exemplary Embodiment Nos. 41-45, wherein i is 0 or 1.

Exemplary Embodiment No. 47. The compound of any one of Exemplary Embodiment Nos. 41-46, wherein R^{J3} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, or or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R.

Exemplary Embodiment No. 48. The compound of Exemplary Embodiment No. 47, wherein R^{J3} is hydrogen, halogen, C₁₋₆ alkyl, 3- to 12-membered heterocyclyl, 5- to 10-membered heteroaryl, or —C(═O)NR^{c}R^{d}, wherein the alkyl, heterocyclyl, or heteroaryl is optionally substituted with one or more R.

Exemplary Embodiment No. 49. The compound of any one of Exemplary Embodiment Nos. 41-48, wherein J¹ is CR¹, and J² is CR^{J2}, wherein R^{J1} and R^{J2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

Exemplary Embodiment No. 50. The compound of Exemplary Embodiment No. 49, wherein R^{J1} and R^{J2} are independently hydrogen or C₁₋₆ alkyl.

Exemplary Embodiment No. 51. The compound of any one of Exemplary Embodiment Nos. 41-48, wherein one of J¹ and J² is N, wherein R^{J1} or R^{J2} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

Exemplary Embodiment No. 52. The compound of any one of Exemplary Embodiment Nos. 1-16, wherein T is of Formula I-3-iv

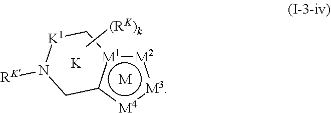

Exemplary Embodiment No. 53. The compound of Exemplary Embodiment No. 52, wherein T is of Formula I-3-iv-a

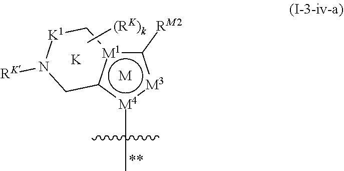

Exemplary Embodiment No. 54. The compound of Exemplary Embodiment No. 52 or 53, wherein Kⁱ is —C(═O)—.

Exemplary Embodiment No. 55. The compound of Exemplary Embodiment No. 52 or 53, wherein K¹ is —CH₂—.

Exemplary Embodiment No. 56. The compound of any one of Exemplary Embodiment Nos. 52-55, wherein at least two of M¹, M³, and M⁴ are N.

Exemplary Embodiment No. 57. The compound of any one of Exemplary Embodiment Nos. 52-55, wherein at least one of M¹, M³, and M⁴ is N.

Exemplary Embodiment No. 58. The compound of any one of Exemplary Embodiment Nos. 52-57, wherein M³ is N.

Exemplary Embodiment No. 59. The compound of any one of Exemplary Embodiment Nos. 52-57, wherein M³ is CR^{M3}, wherein R^{M3} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

Exemplary Embodiment No. 60. The compound of Exemplary Embodiment No. 59, wherein R^{M3} is hydrogen.

Exemplary Embodiment No. 61. The compound of any one of Exemplary Embodiment Nos. 52-60, wherein each R^{K} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R.

Exemplary Embodiment No. 62. The compound of any one of Exemplary Embodiment Nos. 52-61, wherein k is an integer selected from 0 to 2.

Exemplary Embodiment No. 63. The compound of Exemplary Embodiment No. 52, wherein T is of Formula I-3-iv-b

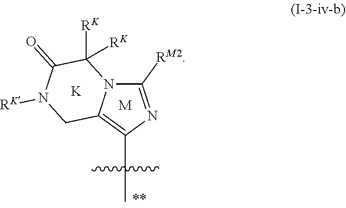

Exemplary Embodiment No. 64. The compound of any one of Exemplary Embodiment Nos. 52-63 wherein each R^{K} is independently hydrogen, or C₁₋₆ alkyl.

Exemplary Embodiment No. 65. The compound of any one of Exemplary Embodiment Nos. 52-64, wherein R^{K′} is hydrogen or C₁₋₆ alkyl.

Exemplary Embodiment No. 66. The compound of any one of Exemplary Embodiment Nos. 52-65, wherein R^{M2} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

Exemplary Embodiment No. 67. The compound of Exemplary Embodiment No. 66, wherein R^{M2} is C₁₋₆ alkyl, C₁₋₆ alkoxy, or 3- to 12-membered heterocyclyl.

Exemplary Embodiment No. 68. The compound of any one of Exemplary Embodiment Nos. 1-16, wherein T is of Formula I-3-v

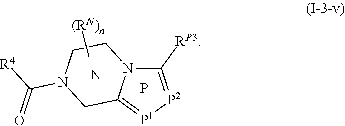

Exemplary Embodiment No. 69. The compound of Exemplary Embodiment No. 68, wherein T is of Formula I-3-v-a

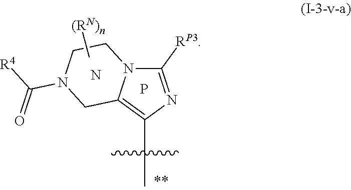

Exemplary Embodiment No. 70. The compound of Exemplary Embodiment No. 68 or 69, wherein each R^{N} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

Exemplary Embodiment No. 71. The compound of any one of Exemplary Embodiment Nos. 68-70, wherein n is 0

Exemplary Embodiment No. 72. The compound of any one of Exemplary Embodiment Nos. 68-71, wherein R⁴ is C₁₋₆ alkyl or C₁₋₆ alkylamino.

Exemplary Embodiment No. 73. The compound of any one of Exemplary Embodiment Nos. 68-72, wherein R^{P3} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R.

Exemplary Embodiment No. 74. The compound of Exemplary Embodiment No. 73, wherein R^{P3} is hydrogen, C₁₋₆ alkyl, C₃₋₁₂ carbocyclyl, 3- to 12-membered heterocyclyl, or —C(═O)NR^{c}R^{d}.

Exemplary Embodiment No. 75. The compound of any one of Exemplary Embodiment Nos. 1-16, wherein T is of Formula I-3-vi

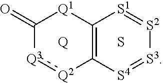

Exemplary Embodiment No. 76. The compound of Exemplary Embodiment No. 75, wherein T is of Formula I-3-vi-a or I-3-vi-b

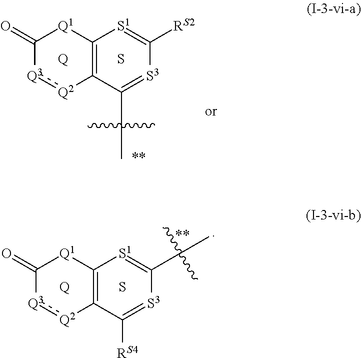

Exemplary Embodiment No. 77. The compound of Exemplary Embodiment No. 75 or 76, wherein R^{S2} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

Exemplary Embodiment No. 78. The compound of Exemplary Embodiment No. 77, wherein R^{S2} is hydrogen, C₁₋₆ alkyl, C₁₋₆ alkoxy, or 3-12-membered heterocyclyl.

Exemplary Embodiment No. 79. The compound of any one of Exemplary Embodiment Nos. 75-78, wherein R^{S4} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

Exemplary Embodiment No. 80. The compound of Exemplary Embodiment No. 79, wherein R^{S4} is hydrogen or C₁₋₆ alkyl.

Exemplary Embodiment No. 81. The compound of any one of Exemplary Embodiment Nos. 75-80, wherein Q¹ is NR^{1′}, wherein R^{Q1′} is hydrogen or C₁₋₆ alkyl.

Exemplary Embodiment No. 82. The compound of any one of Exemplary [symbol] Embodiment Nos. 75-81, wherein-I is a double bond, and Q² is N or CR^{Q2} and Q³ is CR^{Q3}, wherein R^{Q2} and R^{Q3} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl.

Exemplary Embodiment No. 83. The compound of Exemplary Embodiment No. 82, wherein R^{Q2} and R^{Q3} are independently hydrogen or C₁₋₆ alkyl.

Exemplary Embodiment No. 84. The compound of any one of Exemplary Embodiment Nos. 75-81, wherein [symbol] is a single bond, then Q² is C(R^{Q2})₂ and Q³ is NR^{Q3′} or C(R^{Q3})₂, wherein each R^{Q2} and R^{Q3} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl; and R Q3′ ″hydrogen, C₁₋₆ alkyl, C₃₋₄ carbocyclyl, or 3- to 4-membered heterocyclyl.

Exemplary Embodiment No. 85. The compound of Exemplary Embodiment No. 84, wherein each R^{Q2} is hydrogen or C₁₋₆ alkyl, and R^{Q3′} is C₁₋₆ alkyl, and each R^{Q2} and R^{Q3} are independently hydrogen or C₁₋₆ alkyl.

Exemplary Embodiment No. 86. The compound of any one of Exemplary Embodiment Nos. 75-85, wherein S¹ is CR^{S1}, and S³ is CR^{S3}, wherein Rsi and R^{S3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

Exemplary Embodiment No. 87. The compound of Exemplary Embodiment No. 86, wherein each of Rsi and R^{S3} is hydrogen.

Exemplary Embodiment No. 88. The compound of any one of Exemplary Embodiment Nos. 1-87, wherein L″ is absent.

Exemplary Embodiment No. 89. The compound of any one of Exemplary Embodiment Nos. 1-88, wherein Cy¹ is

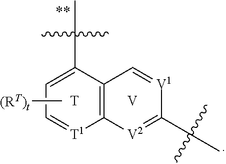

Exemplary Embodiment No. 90. The compound of Exemplary Embodiment No. 89, wherein V¹ is N.

Exemplary Embodiment No. 91. The compound of Exemplary Embodiment No. 89 or 90, wherein T¹ is CR^{T}i and V² is CRV², wherein R^{T}i and R^{V2} are hydrogen.

Exemplary Embodiment No. 92. The compound of Exemplary Embodiment No. 89, wherein Cy¹ is

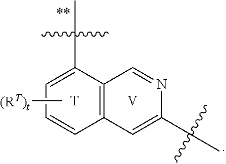

Exemplary Embodiment No. 93. The compound of Exemplary Embodiment No. 92, wherein at least one RT is C₁₋₆ alkyl.

Exemplary Embodiment No. 94. The compound of Exemplary Embodiment No. 92 or 93, wherein t is 0 or 1.

Exemplary Embodiment No. 95. The compound of any one of Exemplary Embodiment Nos. 1-88, wherein Cy¹ is

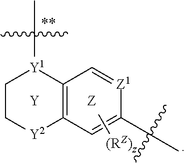

Exemplary Embodiment No. 96. The compound of Exemplary Embodiment No. 95, wherein Y¹ is N.

Exemplary Embodiment No. 97. The compound of Exemplary Embodiment No. 95 or 96, wherein Y² is CH₂ or O.

Exemplary Embodiment No. 98. The compound of any one of Exemplary Embodiment Nos. 95-97, wherein Z¹ is CR^{Z}i

Exemplary Embodiment No. 99. The compound of Exemplary Embodiment No. 95, wherein Cy¹ is

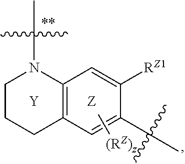

wherein R^{Z1} is C₁₋₆ alkyl substituted with one or more halogen.

Exemplary Embodiment No. 100. The compound of any one of Exemplary Embodiment Nos. 95-99, wherein R^{Z1} is hydrogen or halogen.

Exemplary Embodiment No. 101. The compound of any one of Exemplary Embodiment Nos. 95-97, wherein Z¹ is N.

Exemplary Embodiment No. 102. The compound of any one of Exemplary Embodiment Nos. 95-101, wherein at least one R^{Z} is oxo.

Exemplary Embodiment No. 103. The compound of any one of Exemplary Embodiment Nos. 95-102, wherein z is 0 or 1.

Exemplary Embodiment No. 104. The compound of any one of Exemplary Embodiment Nos. 1-103, wherein L″ is C₁₋₃ alkylene, —C(═O)—, —C(═O)N(R^{L})—, —C(═O)O—, —N(R^{L}), —O—, —S—, or —S(═O)₂—, wherein the alkylene is optionally substituted with one or more R.

Exemplary Embodiment No. 105. The compound of Exemplary Embodiment No. 104, wherein Cy¹ is

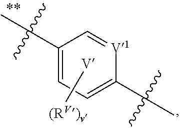

wherein:
** denotes attachment to L″;
V′¹ is CR^{V′1} or N;
R^{V′1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}
each R^{V′} is independently oxo, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; and
v′ is an integer selected from 0 to 5.

Exemplary Embodiment No. 106. The compound of Exemplary Embodiment No. 105, wherein V′¹ is CR^{V′1}, wherein R^{V′1} is hydrogen, halogen, C₁₋₆ alkyl, or C₁₋₆ haloalkyl.

Exemplary Embodiment No. 107. The compound of Exemplary Embodiment No. 104 or 105, wherein each R^{V′} is independently halogen, C₁₋₆ alkyl, or C₁₋₆ haloalkyl, and v′ is 0 or 1.

Exemplary Embodiment No. 108. The compound of any one of Exemplary Embodiment Nos. 1-107, wherein Cy² is pyridinylene optionally substituted with one or more halogen, C₁₋₆ alkyl, or C₁₋₆ alkoxy.

Exemplary Embodiment No. 109. The compound of Exemplary Embodiment No. 108, wherein Cy² is

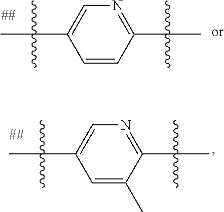

wherein ##denotes attachment to Cy¹.

Exemplary Embodiment No. 110. The compound of any one of Exemplary Embodiment Nos. 1-109, wherein L² is CH₂.

Exemplary Embodiment No. 111. The compound of any one of Exemplary Embodiment Nos. 1-110, wherein L³ is 1,4-phenylene, ethynylene or E-ethylene.

Exemplary Embodiment No. 112. A compound selected from the compounds in Tables 1 and 2 or a pharmaceutically acceptable salt thereof.

Exemplary Embodiment No. 113. A pharmaceutical composition comprising the compound of any one of Exemplary Embodiment Nos. 1-112, and a pharmaceutically acceptable excipient.

Exemplary Embodiment No. 114. A method of degrading a protein in a subject or biological sample comprising administering the compound of any one of Exemplary Embodiment Nos. 1-112 to the subject or contacting the biological sample with the compound of any one of Exemplary Embodiment Nos. 1-112.

Exemplary Embodiment No. 115. Use of the compound of any one of Exemplary Embodiment Nos. 1-112 in the manufacture of a medicament for degrading a protein in a subject or biological sample.

Exemplary Embodiment No. 116. A compound of any one of Exemplary Embodiment Nos. 1-112 for use in degrading a protein in a subject or biological sample.

Exemplary Embodiment No. 117. The method, use, or compound for use of any one of Exemplary Embodiment Nos. 114-116, wherein the protein is p300 or CBP.

Exemplary Embodiment No. 118. A method of treating a p300-mediated disorder comprising administering to a patient in need thereof a compound of any one of Exemplary Embodiment Nos. 1-112.

Exemplary Embodiment No. 119. Use of a compound of any one of Exemplary Embodiment Nos. 1-112 in the manufacture of a medicament for treating a p300-mediated disorder.

Exemplary Embodiment No. 120. A compound of any one of Exemplary Embodiment Nos. 1-112 for use in treating a p300-mediated disorder.

Exemplary Embodiment No. 121. The method, use, or compound for use of any one of Exemplary Embodiment Nos. 118-120, wherein the p300-mediated disorder is cancer, an inflammatory disorder, or an autoimmune disease.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### I. Synthesis and Characterization of Compounds

In the following examples, the chemical reagents were purchased from commercial sources (such as Alfa, Acros, Sigma Aldrich, TCI and Shanghai Chemical Reagent Company), and used without further purification.

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Compound purification was carried out as needed using a variety of traditional methods including, but not limited to, preparative chromatography under acidic, neutral, or basic conditions using either normal phase or reverse phase HPLC or flash columns or Prep-TLC plates.

Flash chromatography was performed on a Biotage Isolera One via column with silica gel particles of 100-200 mesh or 200-300 mesh. Analytical and preparative thin-layer chromatography was performed using silica gel 60 GF254 plates. Normal-phase silica gel chromatography (FCC) was also performed on silica gel (SiO₂) using prepacked cartridges.

Preparative reverse-phase high performance liquid chromatography (RP HPLC) was performed on either:

### Method A

Prep-HPLC with YMC-Actus Triart 18C (5 μm, 20×250 mm), and mobile phase of 5-99% ACN in water (0.1% HCOOH) over 10 min and then hold at 100% ACN for 2 min, at a flow rate of 25 mL/min; or

### Method B

Preparative supercritical fluid high performance liquid chromatography (SFC) was performed either on a Thar 80 Prep-SFC system, or Waters 80Q Prep-SFC system from Waters. The ABPR was set to 100 bar to keep the CO₂ in SF conditions, and the flow rate may verify according to the compound characteristics, with a flow rate ranging from 50 g/min to 70 g/min. The column temperature was ambient temperature.

Nuclear magnetic resonance (NMR) spectra were recorded using Brucker AVANCE NEO 400 MHz at around 20-30° C. unless otherwise specified. The following abbreviations are used: s, singlet; d, doublet; t, triplet; q, quartet; m, multiplet; dd, doublet of doublets; ddd, doublet of doublet of doublets; dt, doublet of triplets; bs, broad signal. Chemical shifts were reported in parts per million (ppm, 6) downfield from tetramethylsilane. It will be understood that for compounds comprising an exchangeable proton, said proton may or may not be visible on an NMR spectrum depending on the choice of solvent used for running the NMR spectrum and the concentration of the compound in the solution.

Mass spectra (MS) were obtained on a SHIMADZU LCMS-2020 MSD using electrospray ionization (ESI) in positive mode unless otherwise indicated. Calculated (calcd.) mass corresponds to the exact mass.

Chemical names were generated using ChemDraw Ultra 12.0, ChemDraw Ultra 14.0, ChemDraw Ultra 20.0 (CambridgeSoft Corp., Cambridge, MA) or ACD/Name Version 10.01 (Advanced Chemistry).

Compounds designated as R* or S* are enantiopure compounds where the absolute configuration was not determined.

### Intermediate 1: Methyl 5-(8-(4,4,5,5-tetramethyl-1,3,2-dioxaborolan-2-yl)isoquinolin-3-yl)picolinate

### Step A: N-(2-Bromobenzyl)-2,2-dimethoxyacetamide

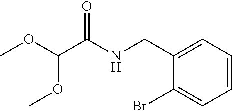 [SMILES: COC(OC)C(=O)NCc1ccccc1Br]

A solution of (2-bromophenyl)methanamine (100 g, 537.6 mmol) in methyl 2,2-dimethoxyacetate (108 g, 806.4 mmol) was stirred at 80° C. for 2 hours in a sealed tube. The suspension was filtered, and the filter cake was washed with EtOAc. The combined filtrates were concentrated under reduced pressure. The residue was purified by flash column chromatography on silica gel (0-10% EtOAc in PE) to afford N-(2-bromobenzyl)-2,2-dimethoxyacetamide (110 g, 71% yield) as a white solid.

LC-MS (ESI): mass calcd. for C₁₁H₁₄BrNO₃: 287.0/289.0; m/z found: 288.0/290.0 [M+H]⁺.

¹H NMR (400 MHz, DMSO-d₆) δ 8.51 (t, J=5.8 Hz, 1H), 7.60 (dd, J=7.9, 0.8 Hz, 1H), 7.40-7.34 (m, 1H), 7.25-7.18 (m, 2H), 4.75 (s, 1H), 4.31 (d, J=6.1 Hz, 2H), 3.33 (s, 6H).

### Step B: 8-Bromoisoquinolin-3-ol

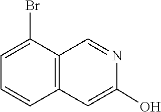 [SMILES: Oc1cc2cccc(Br)c2cn1]

A solution of N-(2-bromobenzyl)-2,2-dimethoxyacetamide (110 g, 383.3 mmol) in H₂SO₄ (100 mL) was stirred at 100° C. for 4 hours. LCMS showed the reaction was complete. The resulting solution was added dropwise into ice water and basified with sat. NaHCO₃to pH=7. The suspension was filtered, the filter cake was washed with water and dried under vacuum to give 8-bromoisoquinolin-3-ol (60 g, 70% yield) as a yellow solid.

LC-MS (ESI): mass calcd. for C₉H₆BrNO: 223.0/225.0; m/z found: 224/226 [M+H]⁺.

¹H NMR (400 MHz, DMSO-d₆) δ 9.02 (s, 1H), 7.75 (d, J=8.5 Hz, 1H), 7.62 (d, J=0.4 Hz, 1H), 7.49-7.43 (m, 1H), 6.97 (s, 1H).

### Step C: 8-Bromoisoquinolin-3-yl trifluoromethanesulfonate

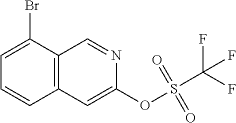 [SMILES: O=S(=O)(Oc1cc2cccc(Br)c2cn1)C(F)(F)F]

To a solution of 8-bromoisoquinolin-3-ol (60 g, 269.0 mmol) in DCM (200 mL) was added TEA (56 mL, 403.6 mmol), followed by Tf20 (54 mL, 322.8 mmol) at 0° C. under N₂. The mixture was stirred at room temperature for 3 hours. The reaction mixture was slowly poured into ice water (300 mL) and extracted with DCM (200 mL×3). The combined organic layers were washed with brine (200 mL×3), dried over Na₂SO₄, and concentrated under reduced pressure. The residue was purified by flash column chromatography on silica gel (0-10%EtOAc in PE) to give 8-bromoisoquinolin-3-yl trifluoromethanesulfonate (35 g, 37% yield) as a yellow solid.

LC-MS (ESI): mass calcd. for C₁₀H₅BrF₃NO₃S: 354.9/356.9; m/z found: 355.9/357.9 [M+H]⁺.

¹H NMR (400 MHz, DMSO-d₆) δ 9.36 (s, 1H), 8.25 (s, 1H), 8.18 (d, J=8.0 Hz, 1H), 8.13 (d, J=8.0 Hz, 1H), 7.82 (t, J=8.0 Hz, 1H).

### Step D: Methyl 5-(8-bromoisoquinolin-3-yl)picolinate

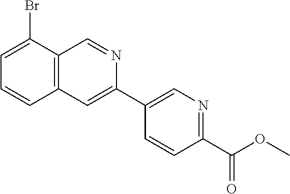 [SMILES: COC(=O)c1ccc(-c2cc3cccc(Br)c3cn2)cn1]

A mixture of 8-bromoisoquinolin-3-yl trifluoromethanesulfonate (6.0 g, 16.9 mmol), methyl 5-(4,4,5,5-tetramethyl-1,3,2-dioxaborolan-2-yl)picolinate (4.9 g, 18.6 mmol), Pd(dppf)Cl₂ (1.2 g, 1.7 mmol), and K₃PO₄ (7.2 g, 33.8 mmol) in DMF (200 mL) was stirred at 70° C. under N₂ overnight. The mixture was poured into EtOAc (800 mL) and filtered. The filter cake was washed with EtOAc. The combined filtrates were washed with brine (200 mL×3), dried over Na₂SO₄, and concentrated under reduced pressure. The residue was purified by flash column chromatography on silica gel (0-10% EtOAc in DCM) to give methyl 5-(8-bromoisoquinolin-3-yl)picolinate (3.0 g, 52% yield) as a yellow solid.

LC-MS (ESI): mass calcd. for C₁₆H₁₁BrN₂O₂: 342.0/344.0; m/z found: 343.0/345.0 [M+H]⁺.

### Step E: Methyl 5-(8-(4,4,5,5-tetramethyl-1,3,2-dioxaborolan-2-yl)isoquinolin-3-yl)picolinate

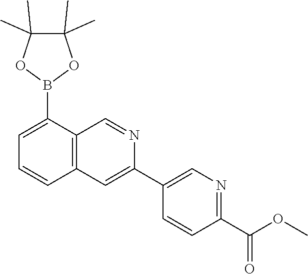

A mixture of methyl 5-(8-bromoisoquinolin-3-yl)picolinate (3.0 g, 8.8 mmol), 4,4,4′,4′,5,5,5′,5′-octamethyl-2,2′-bi(1,3,2-dioxaborolane) (4.5 g, 17.6 mmol), Pd(dppf)Cl₂ (652 mg, 0.9 mmol), and AcOK (2.6 g, 26.4 mmol) in 1,4-dioxane (60 mL) was stirred at 100° C. under N₂ for 3 hours. The mixture was filtered, and the filter cake was washed with EtOAc. The combined filtrates were concentrated under reduced pressure. The residue was purified by flash column chromatography on silica gel (0-10% EtOAc in DCM) to give methyl 5-(8-(4,4,5,5-tetramethyl-1,3,2-dioxaborolan-2-yl)isoquinolin-3-yl)picolinate (2.2 g, 65% yield) as a brown solid.

LC-MS (ESI): mass calcd. for C₂₂H₂₃BN₂O₄: 390.2; m/z found: 391.2 [M+H]⁺.

¹H NMR (400 MHz, DMSO-d₆) δ 10.09 (s, 1H), 9.57 (d, J=1.6 Hz, 1H), 8.78 (dd, J=8.2, 2.3 Hz, 1H), 8.72 (s, 1H), 8.24-8.18 (m, 2H), 8.14 (dd, J=6.9, 1.2 Hz, 1H), 7.87 (dd, J=8.2, 6.9 Hz, 1H), 3.94 (s, 3H), 1.43 (s, 12H).

### Intermediate 2. Lithium 5-(8-(7-isopropyl-1,3-dimethyl-2-oxo-2,3-dihydro-1H-benzo[d]imidazol-5-yl)isoquinolin-3-yl)picolinate

### Step A: 6-Bromo-1,3-dimethyl-4-(prop-1-en-2-yl)-1,3-dihydro-2H-benzo[d]imidazol-2-one

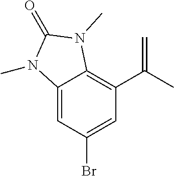 [SMILES: C=C(C)c1cc(Br)cc2c1n(C)c(=O)n2C]

To a mixture of 6-bromo-4-iodo-1,3-dimethyl-1,3-dihydro-2H-benzo[d]imidazol-2-one (1.7 g, 4.6 mmol, 1.0 eq.), 4,4,5,5-tetramethyl-2-(prop-1-en-2-yl)-1,3,2-dioxaborolane (924 mg, 5.5 mmol, 1.2 eq.) and Pd(dppf)Cl₂ (333 mg, 460 μmol, 0.1 eq.) in 1,4-dioxane (20 mL) and H₂O (2 mL) was added Cs₂CO₃ (4.5 g, 13.8 mmol, 3.0 eq.). The mixture was stirred at 90° C. for 2 hours under N₂. The resulting solution was diluted with 50 mL of water and extracted with EtOAc (3×40 mL). The combined organic layers were washed with brine, dried over Na₂SO₄, and concentrated under reduced pressure. The residue was purified by flash column chromatography on silica gel (0-30% EtOAc in PE) to give 6-bromo-1,3-dimethyl-4-(prop-1-en-2-yl)-1,3-dihydro-2H-benzo[d]imidazol-2-one (965 mg, 76% yield) as a white solid.

LC-MS (ESI): mass calcd. for C₁₂H₁₃BrN₂O: 280/282; m/z found: 281/283 [M+H]⁺.

### Step B: 6-Bromo-4-isopropyl-4,3-dimethyl-4,3-dihydro-2H-benzo[d]imidazol-2-one

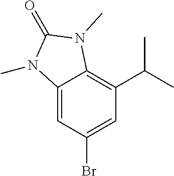 [SMILES: CC(C)c1cc(Br)cc2c1n(C)c(=O)n2C]

A mixture of 6-bromo-1,3-dimethyl-4-(prop-1-en-2-yl)-1,3-dihydro-2H-benzo[d]imidazol-2-one (965 mg, 3.4 mmol) and Wilkinson's catalyst (193 mg, 209 μmol) in EtOH (15 mL) was stirred at 60° C. for 3 hours under H₂ atmosphere. The resulting solution was concentrated under reduced pressure. The residue was purified by flash column chromatography on silica gel (0-30% EtOAc in PE) to give 6-bromo-4-isopropyl-1,3-dimethyl-1,3-dihydro-2H-benzo[d]imidazol-2-one (811 mg, 83% yield) as a white solid.

LC-MS (ESI): mass calcd. for C₁₂H₁₅BrN₂O: 282/284; m/z found: 283/285 [M+H]⁺.

¹H NMR (400 MHz, DMSO-d₆) δ 7.25 (d, J=1.9 Hz, 1H), 7.10 (d, J=1.9 Hz, 1H), 3.63-3.59 (m, 1H), 3.54 (s, 3H), 3.30 (s, 3H), 1.25 (d, J=6.8 Hz, 6H).

### Step C: Methyl 5-(8-(7-isopropyl-1,3-dimethyl-2-oxo-2,3-dihydro-]H-benzo[d]imidazol-5-yl)isoquinoin-3-yl)picoiinate

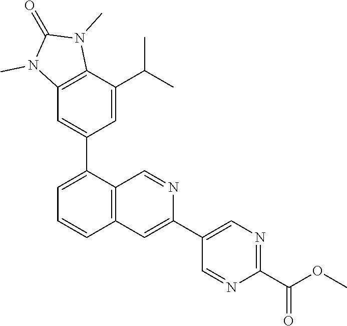

To solution of 6-bromo-4-isopropyl-1,3-dimethyl-1,3-dihydro-2H-benzo[d]imidazol-2-one (90.0 mg, 318 μmol, 1.0 eq.) in 1,4-dioxane (10 mL) and water (1 mL) was added methyl 5-(8-(4,4,5,5-tetramethyl-1,3,2-dioxaborolan-2-yl)isoquinolin-3-yl)picolinate (149 mg, 381 μmol, 1.2 eq., Intermediate 1), K₂CO₃ (132 mg, 953 μmol, 3.0 eq.) and Pd(dppf)Cl2 (23.3 mg, 31.8 μmol, 0.1 eq.). The reaction mixture was stirred for 2 hours at 95° C. The reaction was concentrated under reduced pressure. The residue was purified by flash column chromatography on silica gel (0-50% EtOAc in PE) to give methyl 5-(8-(7-isopropyl-1,3-dimethyl-2-oxo-2,3-dihydro-1H-benzo[d]imidazol-5-yl)isoquinolin-3-yl)picolinate (110 mg, 74% yield) as a white solid.

LC-MS (ESI): mass calcd. for C₂₈H₂₆N₄O₃: 466.2; m/z found: 467.2 [M+H]⁺.

### Step D: Lithium 5-(8-(7-isopropyl-1,3-dimethyl-2-oxo-2,3-dihydro-]H-benzo[d]imidazol-5-yl)isoquinolin-3-yl)picolinate

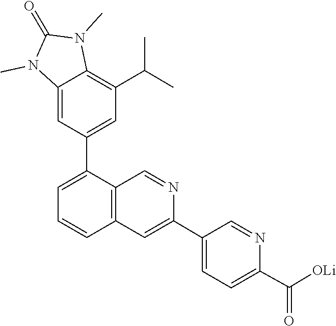

To solution of methyl 5-(8-(7-isopropyl-1,3-dimethyl-2-oxo-2,3-dihydro-1H-benzo[d]imidazol-5-yl)isoquinolin-3-yl)picolinate (30 mg, 64.3 μmol, 1.0 eq.) in THF (3 mL) and water (3 mL) was added LiGH (3 mg, 129 μmol, 2.0 eq.). The reaction mixture was stirred at room temperature for 1 hour and concentrated under reduced pressure to give lithium 5-(8-(7-isopropyl-1,3-dimethyl-2-oxo-2,3-dihydro-1H-benzo[d]imidazol-5-yl)isoquinolin-3-yl)picolinate as a lithium salt.

LC-MS (ESI): mass calcd. for C₂₇H₂₄N₄O₃: 452.2; m/z found: 453.2 [M+H]⁺.

### Intermediate 3. Lithium 5-(7-(difluoromethyl)-1-(7-isopropyl-1,3-dimethyl-2-oxo-2,3-dihydro-1H-benzo[d]imidazol-5-yl)-1,2,3,4-tetrahydroquinolin-6-yl)picolinate

### Step A: Methyl 5-(7-(difluoromethyl)_I-(7-isopropyl-],3-dimethyl-2-oxo-2,3-dihydro-]H-benzo[d]imidazol-5-yl)-1,2,3,4-tetrahydroquinolin-6-yl)picolinate

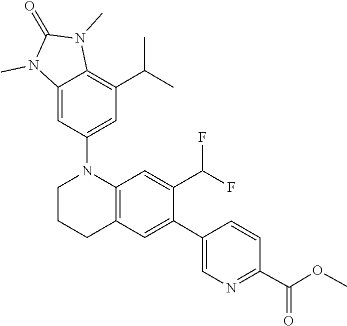

A mixture of methyl 5-(7-(difluoromethyl)-1,2,3,4-tetrahydroquinolin-6-yl)picolinate (150 mg, 471 μmol, 1.0 eq.; Intermediate 5), 6-bromo-4-isopropyl-1,3-dimethyl-1,3-dihydro-2H-benzo[d]imidazol-2-one (147 mg, 518 μmol, 1.1 eq.; Intermediate 2, Step B), RuPhos Pd G1 (103 mg, 141 μmol, 0.3 eq.), RuPhos (144 mg, 283 μmol, 0.6 eq.) and Cs₂CO₃ (461 mg, 1.4 mmol, 3.0 eq.) in 1,4-dioxane (5 mL) was stirred at 100° C. for 3 hours under N₂. The resulting solution was diluted with 40 mL of water and extracted with EtOAc (50 mL×3). The combined organic layers were washed with brine (50 mL), dried over Na₂SO₄, and concentrated under reduced pressure. The residue was purified by flash column chromatography on silica gel (0-80% EtOAc in PE) to give methyl 5-(7-(difluoromethyl)-1-(7-isopropyl-1,3-dimethyl-2-oxo-2,3-dihydro-1H-benzo[d]imidazol-5-yl)-1,2,3,4-tetrahydroquinolin-6-yl)picolinate (100 mg, 41% yield) as a yellow solid.

LC-MS (ESI): mass calcd. for C₂₉H₃₀F2N₄O₃: 520.2; m/z found: 521 [M+H]⁺.

### Step B: Lithium 5-(7-(difluoromethyl)_I-(7-isopropyl-1,3-dimethyl-2-oxo-2,3-dihydro-]H-benzo[d]imidazol-5-yl)-1,2,3,4-tetrahydroquinolin-6-yl)picolinate

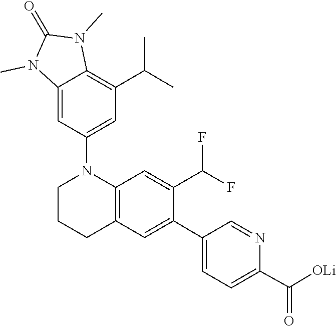

To a solution of methyl 5-(7-(difluoromethyl)-1-(7-isopropyl-1,3-dimethyl-2-oxo-2,3-dihydro-1H-benzo[d]imidazol-5-yl)-1,2,3,4-tetrahydroquinolin-6-yl)picolinate (150 mg, 288 μmol, 1 eq.) in THF (2 mL) and H₂O(2 mL) was added LiGH (8 mg, 288 μmol, 1.2 eq.) at 25° C.

The mixture was stirred at 25° C. for 30 mins. The reaction solution was removed under reduced pressure to give lithium 5-(7-(difluoromethyl)-1-(7-isopropyl-1,3-dimethyl-2-oxo-2,3-dihydro-1H-benzo[d]imidazol-5-yl)-1,2,3,4-tetrahydroquinolin-6-yl)picolinate (145 mg, crude) as a yellow solid.

LC-MS (ESI): mass calcd. for C₂₈H₂₈F₂N₄O₃: 506.2; m/z found: 507 [M+H]⁺.

### Intermediate 4. Lithium 5-(8-(7-ethyl-1,3-dimethyl-2-oxo-1,2-dihydroquinolin-5-yl)isoquinolin-3-yl)picolinate

### Step A: 5-Methoxy-1,3-dimethyl-7-vinyiquinolin-2(]H)-one

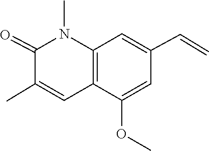 [SMILES: C=Cc1cc(OC)c2cc(C)c(=O)n(C)c2c1]

To a mixture of 7-bromo-5-methoxy-1,3-dimethylquinolin-2(1H)-one (3.5 g, 12.4 mmol, 1.0 eq.), potassium trifluoro(vinyl)borate (8.3 g, 62.0 mmol, 5.0 eq.), and Pd(dppf)Cl2 (1.8 g, 2.5 mmol, 0.2 eq.) in 1,4-dioxane (20 mL) and H₂O(2 mL) was added Na₂CO₃ (3.9 g, 37.2 mmol, 3.0 eq.). The mixture was then stirred at 100° C. for 40 min under N₂. The resulting solution was diluted with 100 mL of water and extracted with EtOAc (50 mL×3). The combined organic layers were washed with brine (50 mL), dried over Na₂SO₄, and concentrated under reduced pressure. The residue was purified by silica gel chromatography (100-200 mesh silica gel, 0-20% EtOAc in PE) to give 5-methoxy-1,3-dimethyl-7-vinylquinolin-2(1H)-one (2.5 g, 88% yield) as a yellow solid.

LC-MS (ESI): mass calcd. for C₁₄H₁₅NO₂, 229.1; m/z found, 230.1 [M+H]⁺.

### Step B: 7-Ethyl-5-methoxy-1,3-dimethylquinolin-2(]H)-one

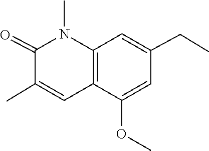 [SMILES: CCc1cc(OC)c2cc(C)c(=O)n(C)c2c1]

To a solution of 5-methoxy-1,3-dimethyl-7-vinylquinolin-2(1H)-one (2.5 g, 10.9 mmol, 1.0 eq.) in EtOH (100 mL) was added Pd/C (1.21 g, wt=10%) at room temperature. The mixture was purged with H₂ three times and stirred at room temperature for 2 hours. The resulting solution was filtered and concentrated under reduced pressure to give 7-ethyl-5-methoxy-1,3-dimethylquinolin-2(1H)-one (2.5 g, crude) as a yellow solid.

LC-MS (ESI): mass calcd. for C₁₄H₁₇NO₂, 231.1; m/z found, 232.1 [M+H]⁺.

### Step C: 7-Ethyl-5-hydroxy-1,3-dimethylquinolin-2(]H)-one

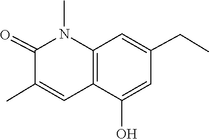 [SMILES: CCc1cc(O)c2cc(C)c(=O)n(C)c2c1]

To mixture of 7-ethyl-5-methoxy-1,3-dimethylquinolin-2(1H)-one (2.5 g, 10.8 mmol, 1.0 eq.) in DMF (100 mL) was added EtSNa (9.1 g, 108 mmol, 10 eq.). The mixture was stirred at 110° C. for 16 hours. The resulting solution was diluted with 100 mL of water and extracted with EtOAc (50 mL×3). The combined organic layers were washed with brine (30 mL×3), dried over Na₂SO₄, and concentrated under reduced pressure to give 7-ethyl-5-hydroxy-1,3-dimethylquinolin-2(1H)-one (2.3 g, crude) as a yellow solid.

LC-MS (ESI): mass calcd. for C₁₃H₁₅NO₂, 217.1; m/z found, 218.1 [M+H]⁺.

### Step D: 7-Ethyl-, 3-dimethyl-2-oxo-1,2-dihydroquinolin-5-yl trifluoromethanesulfonate

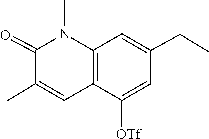 [SMILES: CCc1cc(OS(=O)(=O)C(F)(F)F)c2cc(C)c(=O)n(C)c2c1]

A solution of 7-ethyl-5-hydroxy-1,3-dimethylquinolin-2(1H)-one (2.3 g, 9.9 mmol, 1.0 eq.) and pyridine (2.5 mL, 29.8 mmol, 3.0 eq.) in DCM (30 mL) was stirred at 0° C. Then, Tf₂O (3.4 mL, 19.9 mmol, 2.0 eq.) was added dropwise and the mixture was stirred at 0° C. for 1 hour. The resulting solution was diluted with 50 mL of water and extracted with EtOAc (30 mL×3). The combined organic layers were washed with brine (20 mL×3), dried over Na₂SO₄, and concentrated under reduced pressure. The residue was purified by silica gel chromatography (100-200 mesh silica gel, 0-40% EtOAc in PE) to give 7-ethyl-1,3-dimethyl-2-oxo-1,2-dihydroquinolin-5-yl trifluoromethanesulfonate (1.3 g, 35% yield) as a yellow solid.

LC-MS (ESI): mass calcd. for C₁₄H₁₄F₃NO₄S, 349.1; m/z found, 350.2 [M+H]⁺.

### Step E: Methyl 5-(8-(7-ethyl-1,3-dimethyl-2-oxo-],2-dihydroquinolin-5-yl)isoquinolin-3-yl)picolinate

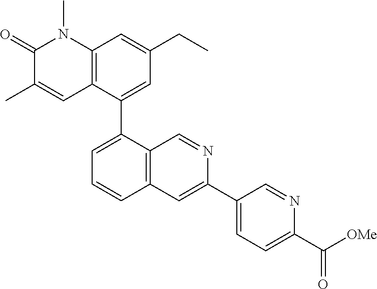 [SMILES: CCc1cc(-c2cccc3cc(-c4ccc(C(=O)OC)nc4)ncc23)c2cc(C)c(=O)n(C)c2c1]

To a mixture of 7-ethyl-1,3-dimethyl-2-oxo-1,2-dihydroquinolin-5-yl trifluoromethanesulfonate (500 mg, 1.4 mmol, 1.0 eq.) and methyl 5-(8-(4,4,5,5-tetramethyl-1,3,2-dioxaborolan-2-yl)isoquinolin-3-yl)picolinate (614 mg, 1.6 mmol, 1.1 eq.; Intermediate 1) in 1,4-dioxane (16 mL) and H₂O(0.8 mL) was added K₂CO₃ (593 mg, 4.3 mmol, 3.0 eq.) and X-Phos Pd G2 (113 mg, 143 μmol, 0.1 eq.) at 25° C. The mixture was stirred at 90° C. for 3 hours under N₂. The resulting solution was diluted with 20 mL of water and extracted with EtOAc (30 mL×3). The combined organic layers were washed with brine (20 mL), dried over Na₂SO₄, and concentrated under reduced pressure. The residue was purified by silica gel chromatography (100-200 mesh silica gel, 0-10% MeOH in DCM) to give methyl 5-(8-(7-ethyl-1,3-dimethyl-2-oxo-1,2-dihydroquinolin-5-yl)isoquinolin-3-yl)picolinate (420 mg, 63% yield) as a yellow solid.

LC-MS (ESI): mass calcd. for C₂₉H₂₅N₃O₃, 463.2; m/z found, 464.1 [M+H]⁺.

### Step F: Lithium 5-(8-(7-ethyl-1,3-dimethyl-2-oxo-1,2-dihydroquinolin-5-yl)isoquinolin-3-yl)picolinate

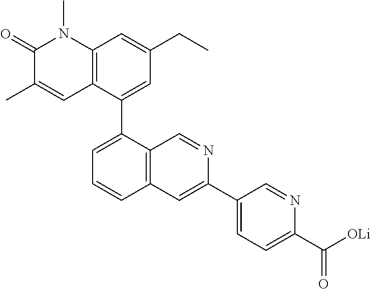

To a solution of methyl 5-(8-(7-ethyl-1,3-dimethyl-2-oxo-1,2-dihydroquinolin-5-yl)isoquinolin-3-yl)picolinate (420 mg, 0.9 mmol, 1.0 eq.) in THF (6 mL) and H₂O(3 mL) was added LiGH (22 mg, 0.9 mmol, 1.0 eq.) at room temperature. The mixture was stirred at room temperature for 1 hour. Then the mixture was concentrated under reduced pressure to give lithium 5-(8-(7-ethyl-1,3-dimethyl-2-oxo-1,2-dihydroquinolin-5-yl)isoquinolin-3-yl)picolinate (380 mg, crude) as a white solid.

LC-MS (ESI): mass calcd. for C₂₈H₂₃N₃O₃, 449.2; m/z found, 450.5 [M+H]⁺.

### Intermediate 5: Methyl 5-(7-(difluoromethyl)-1,2,3,4-tetrahydroquinolin-6-yl)picolinate

### Step A: Quinoline-7-carbaldehyde

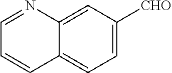 [SMILES: O=Cc1ccc2cccnc2c1]

To a mixture of 7-methylquinoline (25 g, 175 mmol) was added SeO2 (38.7 g, 349.2 mmol) in portions over 5 min at room temperature. The mixture was stirred at 160° C. for 3 hours under N₂ atmosphere. The cooled mixture was diluted with DCM (200 mL) and filtered. The filtrate was concentrated under reduced pressure. The residue was purified by flash chromatography (0-10%EtOAc in PE) to give quinoline-7-carbaldehyde (13 g, 47% yield) as a yellow solid.

LC-MS (ESI): mass calcd. for C₁₀H₇NO: 157; m/z found: 158 [M+H]⁺.

### Step B: 7-(Difluoromethyl)quinoline

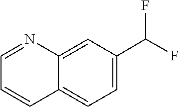 [SMILES: FC(F)c1ccc2cccnc2c1]

To a stirred mixture of quinoline-7-carbaldehyde (5 g, 31.8 mmol) in DCM (10 mL) was added DAST (12.6 mL, 95.4 mmol) dropwise at −20° C. The reaction mixture was stirred overnight at room temperature. The mixture was poured into aq. NaHCO₃at 0° C. and extracted with DCM (30 mL×3). The combined organic layers were washed with brine, dried over anhydrous Na₂SO₄, and concentrated under reduced pressure. The residue was purified by flash chromatography (0-10% EtOAc in PE) to give 7-(difluoromethyl)quinoline (3.6 g, 63% yield) as a yellow solid.

LC-MS (ESI): mass calcd. for C₁₀H₇F₂N: 179; m/z found: 180 [M+H]⁺.

### Step C: 7-(Difluoromethyl)-1,2,3,4-tetrahydroquinoline

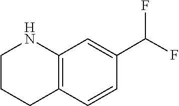 [SMILES: FC(F)c1ccc2c(c1)NCCC2]

To a solution of 7-(difluoromethyl)quinoline (5 g, 27.9 mmol) and NaBH₃CN (9.0 g, 140 mmol) in MeOH (100 mL) was added BF3-Et20 (6.9 mL, 55.8 mmol) dropwise at 0° C. The reaction mixture was stirred overnight at 90° C. under N₂ atmosphere. The mixture was poured into saturated aq. NaHCO₃ (50 mL) and extracted with DCM (30 mL×3). The combined organic layers were washed with brine, dried over anhydrous Na₂SO₄, and concentrated under reduced pressure. The residue was purified by flash chromatography (0-10% EtOAc in PE) to give 7-(difluoromethyl)-1,2,3,4-tetrahydroquinoline (3.6 g, 69% yield) as a brown oil.

LC-MS (ESI): mass calcd. for C₁₀H₁₁F₂N: 183; m/z found: 184 [M+H]⁺.

### Step D: 6-Bromo-7-(difluoromethyl)-1,2,3,4-tetrahydroquinoline

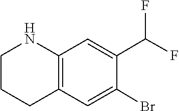 [SMILES: FC(F)c1cc2c(cc1Br)CCCN2]

To a stirred mixture of 7-(difluoromethyl)-1,2,3,4-tetrahydroquinoline (1 g, 5.40 mmol) in DCM (50 mL) was added NBS (860 mg, 4.85 mmol) at 0° C. The reaction mixture was stirred at 0° C. for 30 mins under N₂ atmosphere. The mixture was diluted with water (30 mL) and extracted with DCM (20 mL×3). The combined organic layers were washed with brine, dried over anhydrous Na₂SO₄, and concentrated under reduced pressure. The residue was purified by flash chromatography (0-3%EtOAc in PE) to give 6-bromo-7-(difluoromethyl)-1,2,3,4-tetrahydroquinoline (750 mg, 52% yield) as a colorless oil.

LC-MS (ESI): mass calcd. for C₁₀H₁₀BrF₂N: 261/263; m/z found: 262/264 [M+H]⁺.

### Step E: Methyl 5-(7-(difluoromethyl)-1,2,3,4-tetrahydroquinolin-6-yl)picolinate

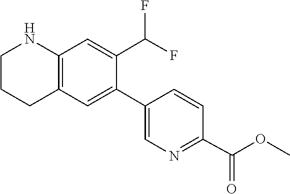 [SMILES: COC(=O)c1ccc(-c2cc3c(cc2C(F)F)NCCC3)cn1]

A mixture of 6-bromo-7-(difluoromethyl)-1,2,3,4-tetrahydroquinoline (750 mg, 2.8 mmol), methyl 5-(4,4,5,5-tetramethyl-1,3,2-dioxaborolan-2-yl)picolinate (736 mg, 2.8 mmol), Pd(dppf)Cl₂ (207 mg, 0.28 mmol) and K₂CO₃ (783 mg, 5.6 mmol) in 1,4-dioxane (20 mL) was stirred at 100° C. under N₂ atmosphere for 3 hours. The cooled mixture was diluted with water (30 mL) and extracted with EtOAc (20 mL×3). The combined organic layers were washed with brine, dried over anhydrous Na₂SO₄, and concentrated under reduced pressure. The residue was purified by flash chromatography (0-10% MeOH in DCM) to give methyl 5-(7-(difluoromethyl)-1,2,3,4-tetrahydroquinolin-6-yl)picolinate (400 mg, 53% yield) as a brown solid.

LC-MS (ESI): mass calcd. for C₁₇H₁₆F₂N₂O₂: 318.1; m/z found: 319 [M+H]⁺.

### Intermediate 6. 3-((3-(3-Aminoprop-1-yn-1-yl)phenyl)amino)piperidine-2,6-dione, hydrochloride salt

### Step A: 3-((3-Iodophenyl)amino)piperidine-2,6-dione

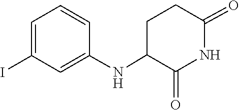 [SMILES: O=C1CCC(Nc2cccc(I)c2)C(=O)N1]

A solution of 3-iodoaniline (16 g, 73.0 mmol, 1.0 eq.) in DMF (40 mL) was treated with 3-bromopiperidine-2,6-dione (18.2 g, 95.0 mmol, 1.3 eq.) and NaHCO₃ (18.4 g, 219.2 mmol, 3.0 eq.) at room temperature. The mixture was stirred at 65° C. for 16 hours. The resulting solution was diluted with 200 mL of H₂O and extracted with EtOAc (150 mL×3). The combined organic layers were washed with brine (200 mL), dried over Na₂SO₄, and concentrated under reduced pressure. The residue was purified by flash column chromatography on silica gel (100-200 mesh silica gel, 0-45% EtOAc in PE) to give 3-((3-iodophenyl)amino)piperidine-2,6-dione (4.8 g, 20% yield) as a off-white solid.

LC-MS (ESI): mass calcd. for C₁₁H₁₁IN₂O₂, 330.0; m/z found, 331 [M+H]⁺.

¹H NMR (400 MHz, DMSO-d₆) δ 10.78 (s, 1H), 7.03 (d, J=1.8 Hz, 1H), 6.93-6.79 (m, 2H), 6.69-6.58 (m, 1H), 6.08 (d, J=8.0 Hz, 1H), 4.41-4.26 (m, 1H), 2.81-2.68 (m, 1H), 2.60-2.52 (m, 1H), 2.10-2.02 (m, 1H), 1.95-1.80 (m, 1H).

### Step B: tert-Butyl (3-(3-((2,6-dioxopiperidin-3-yl)amino)phenyl)prop-2-yn-1-yl)carbamate

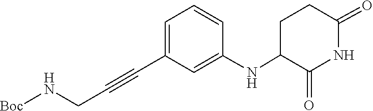 [SMILES: CC(C)(C)OC(=O)NCC#Cc1cccc(NC2CCC(=O)NC2=O)c1]

A solution of 3-((3-iodophenyl)amino)piperidine-2,6-dione (3 g, 9.1 mmol, 1.0 eq.) in DMF (30 mL) was treated with tert-butyl prop-2-yn-1-ylcarbamate (1.7 g, 10.9 mmol, 1.2 eq.), TEA (10 mL, 72.7 mmol, 8.0 eq.), CuI (346 mg, 1.8 mmol, 0.2 eq.), and Pd(PPh₃)Cl₂ (1.2 g, 1.8 mmol, 0.2 eq.) at room temperature. The mixture was stirred at 100° C. under N₂ for 6 hours. The resulting solution was diluted with 150 mL of H₂O and extracted with EtOAc (50 mL×3). The combined organic layers were washed with brine (80 mL), dried over Na₂SO₄, and concentrated under reduced pressure. The residue was purified by flash column chromatography on silica gel (100-200 mesh silica gel, 0-45% EtOAc in PE) to give tert-butyl (3-(3-((2,6-dioxopiperidin-3-yl)amino)phenyl)prop-2-yn-1-yl)carbamate (1.4 g, 43% yield) as a white solid.

LC-MS (ESI): mass calcd. for C₁₉H₂₃N₃O₄, 357.2; m/z found, 302.4 [M+H-tBu]+.

¹H NMR (400 MHz, DMSO-d₆) δ 10.78 (s, 1H), 7.36-7.25 (m, 1H), 7.09-7.02 (m, 1H), 6.70-6.58 (m, 3H), 6.00 (d, J=7.9 Hz, 1H), 4.42-4.33 (m, 1H), 3.94 (d, J=5.6 Hz, 2H), 2.79-2.69 (m, 1H), 2.60-2.54 (m, 1H), 2.11-2.02 (m, 1H), 1.92-1.81 (m, 1H), 1.40 (s, 9H).

### Step C: 3-((3-(3-Aminoprop-1-yn-1-yl)phenyl)amino)piperidine-2,6-dione hydrochloride

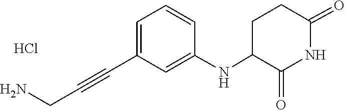 [SMILES: Cl ; NCC#Cc1cccc(NC2CCC(=O)NC2=O)c1]

To a solution of tert-butyl (3-(3-((2,6-dioxopiperidin-3-yl)amino)phenyl)prop-2-yn-1-yl)carbamate (1.4 g, 3.9 mmol, 1.0 eq.) in MeCN (20 mL) was added HCl (4 mL, 4 M in dioxane) and stirred at room temperature for 1 hour. The resulting solution was concentrated under reduced pressure to give 3-((3-(3-aminoprop-1-yn-1-yl)phenyl)amino)piperidine-2,6-dione hydrochloride (1.2 g, 89% yield) as a yellow solid.

LC-MS (ESI): mass calcd. for C₁₄H₁₅N₃O₂, 257.1; m/z found, 258.1 [M+H]⁺.

### Intermediate 7. 5-Bromo-7-(1,1-difluoroethyl)-1,3-dimethyl-3,4-dihydroquinazolin-2(1H)-one

### Step A: 1-(3-Bromo-4-methyl-5-nitrophenyl)ethan--one

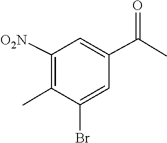

To a solution of 1-(4-methyl-3-nitrophenyl)ethan-1-one (5 g, 27.9 mmol, 1.0 eq.) in H₂SO₄ (55 mL) was added a solution of 1,3-dibromo-1,3,5-triazinane-2,4,6-trione (8 g, 27.9 mmol, 1.0 eq.) in additional H₂SO₄ (15 mL). The reaction was stirred at 25° C. for 16 hours. The reaction solution was slowly poured into 400 mL of ice water and extracted with EtOAc (800 mL).

The organic layer was washed with brine (200 mL), dried over Na₂SO₄, and concentrated under reduced pressure. The residue was purified by flash column chromatography on silica gel (100-200 mesh silica gel, 0-10% EtOAc in PE) to give 1-(3-bromo-4-methyl-5-nitrophenyl)ethan-1-one (6 g, 83% yield) as a yellow solid.

LC-MS (ESI): mass calcd. for C₉H₈BrNO₃, 257.0; m/z found, N/A [M+H]⁺.

¹H NMR (400 MHz, CD30D) 6 8.41 (d, J=1.5 Hz, 1H), 8.32 (d, J=1.3 Hz, 1H), 2.62 (s, 3H), 2.58 (s, 3H).

### Step B: 1-Bromo-5-(1,1-difluoroethyl)-2-methyl-3-nitrobenzene

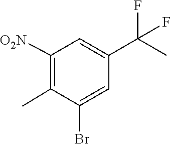

A solution of 1-(3-bromo-4-methyl-5-nitrophenyl)ethan-1-one (3 g, 11.6 mmol, 1.0 eq.) and DAST (23 mL, 46.5 mmol, 4 eq.) in DCE (50 mL) was stirred at 50° C. for 16 hours under N₂. At this point, the reaction was diluted with water (200 mL) and extracted with DCM (100 mL×3). The combined organic layers were washed with brine (50 mL×3), dried over Na₂SO₄, and concentrated under reduced pressure. The residue was purified by flash column chromatography on silica gel (100-200 mesh silica gel, 0-10% EtOAc in PE) to give 1-bromo-5-(1,1-difluoroethyl)-2-methyl-3-nitrobenzene (2 g, 61% yield) as a yellow oil.

No MS signal.

¹H NMR (400 MHz, CD30D) 6 8.04 (s, 1H), 7.96 (s, 1H), 2.56 (s, 3H), 1.96 (t, J=18.5 Hz, 3H).

### Step C: I-Bromo-2-(bromomethyl)-5-(1,1-difluoroethyl)-3-nitrobenzene

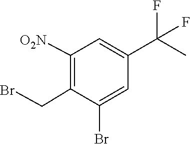 [SMILES: CC(F)(F)c1cc(Br)c(CBr)c([N+](=O)[O-])c1]

A solution of 1-bromo-5-(1,1-difluoroethyl)-2-methyl-3-nitrobenzene (3.2 g, 11.4 mmol, 1.0 eq.), 1-bromopyrrolidine-2,5-dione (2.6 g, 14.9 mmol, 1.3 eq.), and benzoperoxoic acid (158 mg, 1.14 mmol, 0.1 eq.) in CCl₄ (20 mL) was stirred at 80° C. for 16 hours under N₂. The reaction was concentrated under reduced pressure. The residue was purified by silica gel chromatography (100-200 mesh silica gel, 0-15% EtOAc in PE) to give 1-bromo-2-(bromomethyl)-5-(1,1-difluoroethyl)-3-nitrobenzene (4 g, 97% yield) as a yellow solid.

No MS signal.

¹H NMR (400 MHz, CD30D) 6 8.15 (s, 1H), 8.11 (s, 1H), 4.89 (s, 2H), 1.98 (t, J=18.6 Hz, 3H).

### Step D: 1-(2-Bromo-4-(1,1-difluoroethyl)-6-nitrophenyl)-N-methylmethanamine

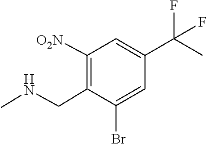 [SMILES: CNCc1c(Br)cc(C(C)(F)F)cc1[N+](=O)[O-]]

To a solution of 1-bromo-2-(bromomethyl)-5-(1,1-difluoroethyl)-3-nitrobenzene (2.2 g, 6.13 mmol, 1.0 eq.) in THF (5.0 mL) was added MeNH2 (30.6 mL, 61.3 mmol, 10.0 eq., 2M in THF). The mixture was stirred at 25° C. for 16 hours under N₂. The reaction was concentrated under reduced pressure. The residue was purified by silica gel chromatography (100-200 mesh silica gel, 0-50% EtOAc in PE) to give 1-(2-bromo-4-(1,1-difluoroethyl)-6-nitrophenyl)-N-methylmethanamine (1.6 g, 85% yield) as a yellow oil.

LC-MS (ESI): mass calcd. for C₁₀H₁₁BrF₂N₂O₂, 308.0; m/z found, 309.0 [M+H]⁺.

### Step E: 3-Bromo-5-(1,1-difluoroethyl)-2-((methylamino)methyl)aniline

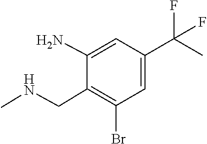

A mixture of 1-(2-bromo-4-(1,1-difluoroethyl)-6-nitrophenyl)-N-methylmethanamine (1.4 g, 4.53 mmol, 1.0 eq.), NH₄Cl (1.2 g, 22.6 mmol, 5 eq.), and iron powder (2.5 g, 45.3 mmol, 10 eq.) in EtOH (10 mL) and H₂O(2 mL) was stirred at 80° C. for 2 hours under N₂. The reaction was filtered, and the filtrate was concentrated in vacuo. The residue was purified by silica gel chromatography (100-200 mesh silica gel, 0-30% MeOH in DCM) to give 3-bromo-5-(1,1-difluoroethyl)-2-((methylamino)methyl)aniline (800 mg, 63% yield) as an white solid.

LC-MS (ESI): mass calcd. for C₁₀H₁₃BrF₂N₂, 278.0; m/z found, 279.0 [M+H]⁺.

### Step F: 5-Bromo-7-(1,1-difluoroethyl)-3-methyl-3,4-dihydroquinazolin-2(1H)-one

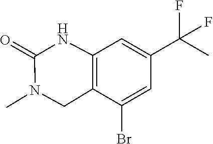 [SMILES: CN1Cc2c(Br)cc(C(C)(F)F)cc2NC1=O]

A solution of 3-bromo-5-(1,1-difluoroethyl)-2-((methylamino)methyl)aniline (700 mg, 2.51 mmol, 1.0 eq.) and CDI (813 mg, 5.02 mmol, 2.0 eq.) in THF (10 mL) was stirred at 80° C. for 20 hours under N₂. The reaction was concentrated under reduced pressure. The residue was purified by Prep-HPLC (C18, 0-70% MeCN in H₂O, 0.1% FA) to give 5-bromo-7-(1,1-difluoroethyl)-3-methyl-3,4-dihydroquinazolin-2(1H)-one (700 mg, 91% yield) as a white solid.

LC-MS (ESI): mass calcd. for C₁₁H₁₁BrF₂N₂O, 304.0; m/z found, 305 [M+H]⁺.

### Step G: 5-Bromo-7-(1,1-difluoroethyl)-1,3-dimethyl-3,4-dihydroquinazolin-2(]H)-one

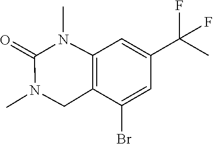 [SMILES: CN1Cc2c(Br)cc(C(C)(F)F)cc2N(C)C1=O]

A solution of 5-bromo-7-(1,1-difluoroethyl)-3-methyl-3,4-dihydroquinazolin-2(1H)-one (400 mg, 1.31 mmol, 1.0 eq.) in DMF (8 mL) was cooled to 0° C., and then treated with NaH (105 mg, 2.62 mmol, 2.0 eq., 60% wt.). The reaction was stirred at 0° C. for 30 mins under N₂. At this point, Mel (127 μL, 1.97 mmol, 1.5 eq.) was added to the mixture dropwise. The mixture was then stirred at 50° C. for 1 hour. The resulting solution was diluted with 20 mL of water and extracted with EtOAc (30 mL×3). The combined organic layers were washed with brine (20 mL×3), dried over Na₂SO₄, and concentrated under reduced pressure. The residue was purified by silica gel chromatography (100-200 mesh silica gel, 0-100% EtOAc in PE) to give 5-bromo-7-(1,1-difluoroethyl)-1,3-dimethyl-3,4-dihydroquinazolin-2(1H)-one (350 mg, 84% yield) as a white solid.

LC-MS (ESI): mass calcd. for C₁₂H₁₃BrF₂N₂O, 318.0; m/z found, 319 [M+H]⁺.

### Intermediate 8. 5-Bromo-7-ethyl-1,3-dimethylquinolin-2(1H)-one

### Step A: 5-Bromo-7-hydroxy-1,3-dimethylquinolin-2(1H)-one

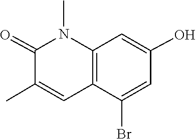

To a solution of 5-bromo-7-methoxy-1,3-dimethylquinolin-2(1H)-one (20.0 g, 70.9 mmol, 1 eq.) in DCM (200 mL) was added BBr3 (55 mL, 567 mmol, 8 eq.). The mixture was stirred at 25° C. for 2 hour under N₂. The resulting solution was quenched with MeOH and filtered to give 5-bromo-7-hydroxy-1,3-dimethylquinolin-2(1H)-one (18.0 g, 67.1 mmol, 95% yield) as a white solid.

LC-MS (ESI): mass calcd. for C₁₁H₁₀BrNO₂, 267.0/269.0; m/z found, 268.1/270.1 [M+H]⁺.

### Step B: 5-Bromo-1,3-dimethyl-2-oxo-1,2-dihydroquinolin-7-yl trifluoromethanesulfonate

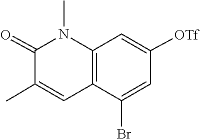 [SMILES: Cc1cc2c(Br)cc(OS(=O)(=O)C(F)(F)F)cc2n(C)c1=O]

To a mixture of 5-bromo-7-hydroxy-1,3-dimethylquinolin-2(1H)-one (16.0 g, 60 mmol, 1 eq.) and pyridine (14.5 mL, 179 mmol, 3 eq.) in DCM (160 mL) was added triflic anhydride (20 mL, 119 mmol, 2 eq.) at 0° C. under N₂ atmosphere. The mixture was stirred at 0° C. for 3 hours. The resulting solution was diluted with 200 mL of water and extracted with EtOAc (200 mL×3). The combined organic layers were washed with brine (200 mL), dried over Na₂SO₄, and concentrated under reduced pressure. The residue was purified by flash column chromatography on silica gel (100-200 mesh silica gel, 0-25% EtOAc in PE) to give 5-bromo-1,3-dimethyl-2-oxo-1,2-dihydroquinolin-7-yl trifluoromethanesulfonate (19.0 g, 47.5 mmol, 80% yield) as a yellow solid.

LC-MS (ESI): mass calcd. for C₁₂H₉BrF₃NO₄S, 398.9/400.9; m/z found, 400/402 [M+H]⁺.

### Step C: 5-Bromo-1,3-dimethyl-7-vinylquinolin-2(]H)-one

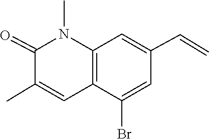 [SMILES: C=Cc1cc(Br)c2cc(C)c(=O)n(C)c2c1]

To a stirred mixture of 5-bromo-1,3-dimethyl-2-oxo-1,2-dihydroquinolin-7-yl trifluoromethanesulfonate (10.0 g, 25 mmol, 1 eq.) and potassium vinyltrifluoroborate (3.8 g, 28.7 mmol, 1.15 eq.) in THF (80 mL) and water (6 mL) was added PdCl₂(dppf) (1.8 g, 2.5 mmol, 0.1 eq.) and K₂CO₃ (10.4 g, 75 mmol, 3 eq.) at room temperature under N₂ atmosphere. The reaction mixture was stirred at 50° C. for 3 hours. The mixture was diluted with water and extracted with EtOAc. The combined organic layers were washed with brine, dried over Na₂SO₄, and concentrated under reduced pressure. The residue was purified by flash column chromatography (50% EtOAc in PE) to give 5-bromo-1,3-dimethyl-7-vinylquinolin-2(1H)-one (5.0 g, 14 mmol, 58% yield, 80% Purity) as a white solid.

LC-MS (ESI): mass calcd. for C₁₃H₁₂BrNO, 277.0/279.0; m/z found, 278.0/280.0 [M+H]⁺.

### Step D: 5-Bromo-7-ethyl-],3-dimethyiquinoiin-2(]H)-one

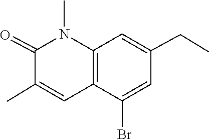 [SMILES: CCc1cc(Br)c2cc(C)c(=O)n(C)c2c1]

To a solution of 5-bromo-1,3-dimethyl-7-vinylquinolin-2(1H)-one (5.0 g, 80% wt., 14.4 mmol, 1 eq.) in EtOH (40 mL) was added Wilkinson's catalyst (1.5 g, 1.62 mmol, 0.11 eq.) at room temperature. The reaction mixture was stirred at 50° C. under H₂ atmosphere for 4 hours.

The mixture was concentrated under reduced pressure. The residue was purified by flash chromatography (20% EtOAc in DCM) to give 5-bromo-7-ethyl-1,3-dimethylquinolin-2(1H)-one (3.0 g, 10.7 mmol, 75% yield).

LC-MS (ESI): mass calcd. for C₁₃H₁₄BrNO, 279.0/281.0; m/z found, 280/282 [M+H]⁺.

### Intermediate 9. 5-Chloro-7-ethyl-1,3-dimethyl-3,4-dihydropyrido[2,3-d]pyrimidin-2(1H)-one

### Step A: tert-Butyl (4,6-dichloropyridin-2-yl)carbamate

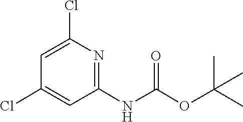 [SMILES: CC(C)(C)OC(=O)Nc1cc(Cl)cc(Cl)n1]

A mixture of 4,6-dichloropyridin-2-amine (10 g, 61.3 mmol, 1.0 eq.), Boc₂O (16 g, 73.6 mmol, 1.2 eq.), DMAP (374 mg, 3.1 mmol, 0.05 eq.), and TEA (16.7 mL, 123 mmol, 2.0 eq.) in DCM (100 mL) was stirred at 25° C. for 16 hours under N₂. The resulting solution was diluted with 300 mL of water and extracted with DCM (100 mL×3). The combined organic layers were washed with brine (50 mL), dried over Na₂SO₄, and concentrated under reduced pressure. The residue was purified by flash column chromatography on silica gel (100-200 mesh silica gel, 0-10% EtOAc in PE) to give tert-butyl (4,6-dichloropyridin-2-yl)carbamate (9.0 g, 56% yield) as a yellow solid.

LC-MS (ESI): mass calcd. for C₁₀H₁₂Cl2N₂O₂, 262.03; m/z found, 263 [M+H]⁺.

### Step B: tert-Butyl (4,6-dichloro-3-formylpyridin-2-yl)carbamate

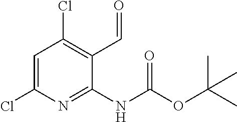 [SMILES: CC(C)(C)OC(=O)Nc1nc(Cl)cc(Cl)c1C=O]

To a solution of tert-butyl (4,6-dichloropyridin-2-yl)carbamate (8.9 g, 33.8 mmol, 1.0 eq.) in THF (100 mL) was added n-BuLi (34 mL, 84.6 mmol, 2.5 eq., 2.5 M in THF) at −78° C. The mixture was stirred at −78° C. for 30 mins. DMF (13 mL, 169 mmol, 5.0 eq.) was then added to this mixture at −78° C. The mixture was stirred at −78° C. for another 30 mins under N₂.

The resulting solution was quenched with 2 N HCl at −78° C. and extracted with EtOAc (100 mL×3). The combined organic layers were washed with brine (100 mL), dried over Na₂SO₄, and concentrated under reduced pressure. The residue was purified by flash column chromatography on silica gel (100-200 mesh silica gel, 0-10% EtOAc in PE) to give tert-butyl (4,6-dichloro-3-formylpyridin-2-yl)carbamate (6 g, 61% yield) as a white solid.

LC-MS (ESI): mass calcd. for C₁₁H₁₂Cl2N₂O₃, 290.0; m/z found, 291 [M+H]⁺.

### Step C: 5,7-Dichloro-3-methyl-3,4-dihydropyrido[2,3-d]pyrimidin-2(]H)-one

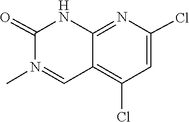

To a solution of tert-butyl (4,6-dichloro-3-formylpyridin-2-yl)carbamate (4 g, 13.7 mmol, 1.0 eq.) in toluene (20 mL) was added MeNH2 (14 mL, 27.5 mmol, 2.0 eq., 2 M in THF) at 25° C. The mixture was stirred at 25° C. for 2 hours under N₂. The reaction mixture was concentrated under reduced pressure. The residue was dissolved in THF (20 mL). NaBH₄ (1 g, 27.5 mmol, 2.0 eq.) was added and stirred at 25° C. for another 16 hours. The mixture was quenched with sat. NaHCO₃ and stirred at 25° C. for 1 hour. The suspension was filtered and the filter cake was washed with water and dried under vacuum to give 5,7-dichloro-3-methyl-3,4-dihydropyrido[2,3-d]pyrimidin-2(1H)-one (2 g, 63% yield) as a white solid.

LC-MS (ESI): mass calcd. for C₈H₇C₁₂N₃O, 231.0; m/z found, 232 [M+H]⁺.

### Step D: 5-Chloro-3-methyl-7-vinyl-3,4-dihydropyrido[2,3-d]pyrimidin-2(]H)-one

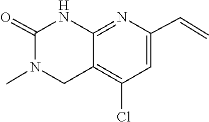

A mixture of 5,7-dichloro-3,4-dihydropyrido[2,3-d]pyrimidin-2(1H)-one (2.5 g, 11.5 mmol, 1.0 eq.), potassium trifluoro(vinyl)borate (2 g, 14.9 mmol, 1.3 eq.), Pd(dppf)Cl₂ (890 mg, 1.15 mmol, 0.1 eq.), and K₂CO₃ (4.8 g, 34.4 mmol, 3.0 eq.) in 1,4-dioxane (25 mL) and H₂O (2.5 mL) was stirred at 80° C. for 5 hours under N₂. The reaction solution was diluted with 50 mL of water and extracted with EtOAc (20 mL×3). The combined organic layers were washed with brine, dried over Na₂SO₄, and concentrated under reduced pressure. Then the residue was purified by flash chromatography (100-200 mesh silica gel, 0-50% EtOAc in PE) to give 5-chloro-3-methyl-7-vinyl-3,4-dihydropyrido[2,3-d]pyrimidin-2(1H)-one(2 g, 78% yield) as a yellow solid.

LC-MS (ESI): mass calcd. for C₁₀H₁₀ClN₃O, 223.1; m/z found, 224 [M+H]⁺.

### Step E: 5-Chloro-7-ethyl-3-methyl-3,4-dihydropyrido[2,3-d]pyrimidin-2(]H)-one

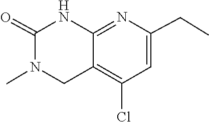

To a solution of 5-chloro-3-methyl-7-vinyl-3,4-dihydropyrido[2,3-d]pyrimidin-2(1H)-one (300 mg, 1.3 mmol, 1.0 eq.) in EtOH (3 mL) was added Wilkinson's catalyst (80 mg) at 25° C. The mixture was stirred at 50° C. for 16 hours under H₂. The resulting solution was concentrated under reduced pressure. The residue was purified by flash column chromatography on silica gel (100-200 mesh silica gel, 0-50% EtOAc in PE) to give 5-chloro-7-ethyl-3-methyl-3,4-dihydropyrido[2,3-d]pyrimidin-2(1H)-one (150 mg, 50% yield) as a yellow solid.

LC-MS (ESI): mass calcd. for C₁₀H₁₂ClN₃O, 225.1; m/z found, 226 [M+H]⁺.

### Step F: 5-Chloro-7-ethyl-],3-dimethyl-3,4-dihydropyrido[2,3-d]pyrimidin-2(]H)-one

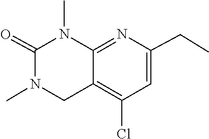 [SMILES: CCc1cc(Cl)c2c(n1)N(C)C(=O)N(C)C2]

To a solution of 5-chloro-7-ethyl-3-methyl-3,4-dihydropyrido[2,3-d]pyrimidin-2(1H)-one (150 mg, 665 μmol, 1.0 eq.) in DMF (5 mL) was added NaH (40 mg, 997 μmol, 1.5 eq., 60% wt.). The mixture was stirred at 0° C. for 30 mins under N₂. Then, Mel (189 mg, 1.4 mmol, 2.0 eq.) was added to this mixture. The mixture was stirred at 30° C. for 30 mins under N₂. The resulting solution was diluted with 50 mL of sat. NH₄Cl and extracted with EtOAc (50 mL×3). The combined organic layers were washed with brine (50 mL), dried over Na₂SO₄, and concentrated under reduced pressure. The residue was purified by flash column chromatography on silica gel (100-200 mesh silica gel, 0-40% EtOAc in PE) to give 5-chloro-7-ethyl-1,3-dimethyl-3,4-dihydropyrido[2,3-d]pyrimidin-2(1H)-one (130 mg, 82% yield) as a yellow solid.

LC-MS (ESI): mass calcd. for C₁₁H₁₄ClN₃O, 239.1; m/z found, 240 [M+H]⁺.

¹H NMR (400 MHz, DMSO-d₆) δ 7.00 (s, 1H), 4.42 (s, 2H), 3.26 (s, 3H), 2.93 (s, 3H), 2.66 (m, J=7.6 Hz, 2H), 1.20 (t, J=7.6 Hz, 3H).

### Intermediate 10. 3-(5-(3-aminoprop-1-yn-1-yl)furo[3,2-b]pyridin-3-yl)piperidine-2,6-dione

### Step A: ethyl (E)-4-((6-bromo-2-iodopyridin-3-yl)oxy)but-2-enoate

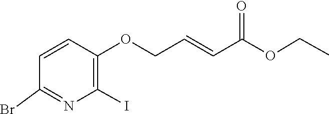 [SMILES: CCOC(=O)/C=C/COc1ccc(Br)nc1I]

To a solution of 6-bromo-2-iodopyridin-3-ol (4 g, 13.3 mmol, 1.0 eq.), ethyl (E)-4-bromobut-2-enoate (2.6 g, 13.3 mmol, 1.0 eq.) in Toluene (60 mL) was added silver carbonate (7.4 g, 26.7 mmol, 2.0 eq.). The mixture was stirred at 80° C. for 3 hours under N₂ atmosphere. The resulting solution was diluted with 50 mL of H₂O and extracted with EtOAc (40 mL×3). The combined organic layers were washed with brine, dried over Na₂SO₄ and concentrated under reduced pressure. The residue was purified by flash column chromatography on silica gel (100-200 mesh silica gel, 0-20% EtOAc in PE) to give ethyl (E)-4-((6-bromo-2-iodopyridin-3-yl)oxy)but-2-enoate (3.5 g, yield 64%) as a white solid.

LC-MS (ESI): mass calcd. for C₁₁H₁₁BrINO₃, 410.9/412.9; m/z found, 411.7/413.7 [M+H]⁺.

### Step B: ethyl 2-(5-bromofuro[3,2-b]pyridin-3-yl)acetate

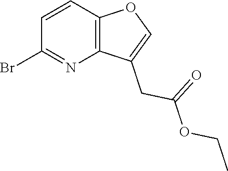 [SMILES: CCOC(=O)Cc1coc2ccc(Br)nc12]

To a solution of ethyl (E)-4-((6-bromo-2-iodopyridin-3-yl)oxy)but-2-enoate (4 g, 9.7 mmol, 1.0 eq.), X-Phos Palladacycle (764 mg, 971 μmol, 0.1 eq.) in DMF (10 mL) was added Na₂CO₃ (2.1 g, 19.4 mmol, 2.0 eq.). The mixture was stirred at 80° C. for 16 hours under N₂ atmosphere. The resulting solution was diluted with 50 mL of H₂O and extracted with EtOAc (40 mL×3). The combined organic layers were washed with brine, dried over Na₂SO₄ and concentrated under reduced pressure. The residue was purified by flash column chromatography on silica gel (100-200 mesh silica gel, 0-20% EtOAc in PE) to give ethyl 2-(5-bromofuro[3,2-b]pyridin-3-yl)acetate (800 mg, yield 29%) as a white solid.

LC-MS (ESI): mass calcd. for C₁₀H₅BrNO₃, 282.9/283.9; m/z found, 283.8/285.8 [M+H]⁺.

### Step C: 3-(5-bromofuro[3,2-b]pyridin-3-yl)piperidine-2,6-dione

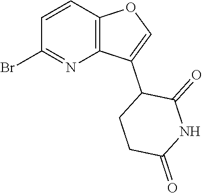 [SMILES: O=C1CCC(c2coc3ccc(Br)nc23)C(=O)N1]

To a solution of methyl 2-(5-bromofuro[3,2-b]pyridin-3-yl)acetate (600 mg, 2.2 mmol, 1.0 eq.), t-BuOK (250 mg, 2.2 mmol, 1.0 eq.) in DMF (10 mL) was added acrylamide (160 mg, 2.2 mmol, 1.0 eq.). The mixture was stirred at room temperature for 2 hours under N₂ atmosphere. The resulting solution was diluted with 40 mL of H₂O and extracted with EtOAc (20 mL×3). The combined organic layers were washed with brine, dried over Na₂SO₄ and concentrated under reduced pressure. The residue was purified by flash column chromatography on silica gel (100-200 mesh silica gel, 0-20% EtOAc in PE) to give 3-(5-bromofuro[3,2-b]pyridin-3-yl)piperidine-2,6-dione (80.0 mg, yield 12%) as a white solid.

LC-MS (ESI): mass calcd. for C₁₂H₉BrN₂O₃, 307.8/309.8; m/z found, 308.7/310.7 [M+H]⁺.

### Step D: tert-butyl (3-(3-(2,6-dioxopiperidin-3-yl)furo[3,2-b]pyridin-5-yl)prop-2-yn-1-yl)carbamate

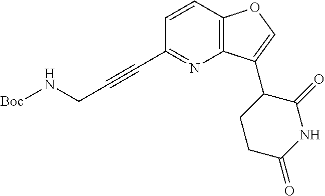 [SMILES: CC(C)(C)OC(=O)NCC#Cc1ccc2occ(C3CCC(=O)NC3=O)c2n1]

To a solution of 3-(5-bromofuro[3,2-b]pyridin-3-yl)piperidine-2,6-dione (50 mg, 162 μmol, 1.0 q.), CuI (6 mg, 32.3 μmol, 0.2 eq.), Pd(PPh₃)C₁₂ (11 mg, 16.2 μmol, 0.1 eq.) in DMF (2 mL) was added TEA (225 μL, 1.62 mmol, 10.0 eq.) and added tert-butyl prop-2-yn-1-ylcarbamate (75 mg, 485 μmol, 3.0 eq.) at 90° C. The mixture was stirred at 90° C. for 3 hours under N₂ atmosphere. The resulting solution was diluted with 10 mL of H₂O and extracted with EtOAc (10 mL×3). The combined organic layers were washed with brine, dried over Na₂SO₄ and concentrated under reduced pressure. The residue was purified by flash column chromatography on silica gel (100-200 mesh silica gel, 0-40% EtOAc in PE) to give tert-butyl (3-(3-(2,6-dioxopiperidin-3-yl)furo[3,2-b]pyridin-5-yl)prop-2-yn-1-yl)carbamate (50 mg, yield 81%) as a brown solid.

LC-MS (ESI): mass calcd. for C₂₀H₂₁N₃O₅, 383.1; m/z found, 384.4 [M+H]⁺.

### Step E: 3-(5-(3-aminoprop-1-yn-1-yl)furo[3,2-b]pyridin-3-yl)piperidine-2,6-dione

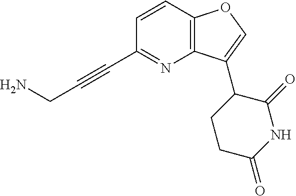 [SMILES: NCC#Cc1ccc2occ(C3CCC(=O)NC3=O)c2n1]

To a solution of tert-butyl (3-(3-(2,6-dioxopiperidin-3-yl)furo[3,2-b]pyridin-5-yl)prop-2-yn-1-yl)carbamate (40 mg, 104.3 μmol, 1.0 eq.) in DCM (3 mL) was added TFA (0.6 mL) at 0° C. The mixture was stirred at room temperature for 1 hour under N₂ atmosphere. The reaction was concentrated under vacuum to give 3-(5-(3-aminoprop-1-yn-1-yl)furo[3,2-b]pyridin-3-yl)piperidine-2,6-dione (28.0 mg, yield 95%) as a brown solid.

LC-MS (ESI): mass calcd. for C₁₅H₁₃N₃O₃, 283.1; m/z found, 284.4 [M+H]⁺.

### Intermediate 11. 5-(5-(3-ethyl-1,7-dimethyl-2-oxo-2,3-dihydro-1H-benzo[d]imidazol-5-yl)-6,7-dihydro-5H-pyrimido[4,5-b][1,4]oxazin-2-yl)picolinic acid

### Step A: methyl 5-(6,7-dihydro-5H-pyrimido[4,5-b][1,4]oxazin-2-yl)-3-methylpicolinate

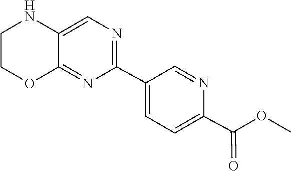 [SMILES: COC(=O)c1ccc(-c2ncc3c(n2)OCCN3)cn1]

A mixture of 2-chloro-6,7-dihydro-5H-pyrimido[4,5-b][1,4]oxazine (450 mg, 2.6 mmol, 1.0 eq.), methyl 3-methyl-5-(4,4,5,5-tetramethyl-1,3,2-dioxaborolan-2-yl)picolinate (1.4 g, 5.2 mmol, 2.0 eq.), Pd(dtbpf)Cl₂ (171 mg, 262 μmol, 0.1 eq.) and K₃PO₄ (1.6 g, 7.8 mmol, 3.0 eq.) in 1,4-Dioxane (5 mL) and H₂O(0.5 mL) was stirred at 100° C. for 1.5 hours under N₂. The resulting solution was diluted with 50 mL of H₂O and extracted with EtOAc (40 mL×3). The combined organic layers were washed with brine, dried over Na₂SO₄, and concentrated under reduced pressure. The residue was purified by flash column chromatography on silica gel (100-200 mesh silica gel, 0-70% EtOAc in PE) to give methyl 5-(6,7-dihydro-5H-pyrimido[4,5-b][1,4]oxazin-2-yl)-3-methylpicolinate (350 mg, yield 46.6%) as a yellow solid.

LC-MS (ESI): mass calcd. for C₁₃H₁₂N₄O₃, 272.1; m/z found, 273.1 [M+H]⁺.

### Step B: methyl 5-(5-(1,3,7-trimethyl-2-oxo-2,3-dihydro-1H-benzo[d]imidazol-5-yl)-6,7-dihydro-5H-pyrimido[4,5-b][1,4]oxazin-2-yl)picolinate

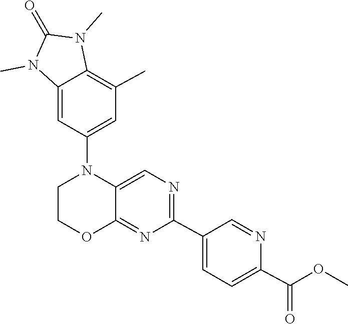

A mixture of methyl 5-(6,7-dihydro-5H-pyrimido[4,5-b][1,4]oxazin-2-yl)-3-methylpicolinate (80 mg, 279 μmol, 1.0 eq.),6-bromo-1,3,4-trimethyl-1,3-dihydro-2H-benzo[d]imidazol-2-one (85 mg, 335 μmol, 1.2 eq.), Ru-Phos (26 mg, 56 μmol, 1.2 eq.), Ru-Phos Pd G1 (41 mg, 56 μmol, 0.2 eq.) and Cs₂CO₃ (273 mg, 838 μmol, 3.0 eq.) in 1,4-Dioxane (2 mL) was stirred at 100° C. for 16 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-100% EtOAc in PE) to give methyl 3-methyl-5-(5-(1,3,7-trimethyl-2-oxo-2,3-dihydro-1H-benzo[d]imidazol-5-yl)-6,7-dihydro-5H-pyrimido[4,5-b][1,4]oxazin-2-yl)picolinate (80 mg, yield 62%) as ayellow solid. LC-MS (ESI): mass calcd. for C₂₃H₂₂N₆O₄, 446.1; m/z found, 447.1 [M+H]⁺.

### Step C: 5-(5-(3-ethyl-1,7-dimethyl-2-oxo-2,3-dihydro-1H-benzo[d]imidazol-5-yl)-6,7-dihydro-5H-pyrimido[4,5-b][1,4]oxazin-2-yl)picolinic acid

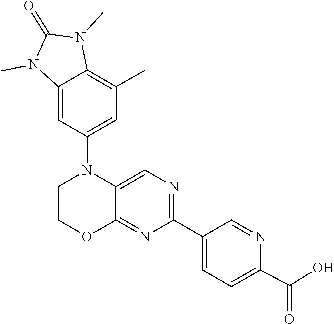 [SMILES: Cc1cc(N2CCOc3nc(-c4ccc(C(=O)O)nc4)ncc32)cc2c1n(C)c(=O)n2C]

Methyl 5-(5-(3-ethyl-1,7-dimethyl-2-oxo-2,3-dihydro-1H-benzo[d]imidazol-5-yl)-6,7-dihydro-5H-pyrimido[4,5-b][1,4]oxazin-2-yl)picolinate (30 mg, 65.1 μmol, 1.0 eq.) in THF (3 mL) and H₂O(1 mL) was added LiGH (6 mg, 130 μmol, 2.0 eq.). The mixture was stirred at 25° C. for 30 mins. The reaction was monitored by LCMS. The resulting solution was dried over anhydrous sodium sulfate and concentrated under reduced pressure to afford 5-(5-(3-ethyl-1,7-dimethyl-2-oxo-2,3-dihydro-1H-benzo[d]imidazol-5-yl)-6,7-dihydro-5H-pyrimido[4,5-b][1,4]oxazin-2-yl)picolinic acid (29 mg, yield 99%) as a yellow solid.

LC-MS (ESI): mass calcd. for C₂₂H₂₀N₆O₄, 432.1; m/z found, 433.1 [M+H]⁺.

### Intermediate 12. 3-(2-(3-aminoprop-1-yn-1-yl)furo[3,2-c]pyridin-4-yl)piperidine-2,6-dione

### Step A: methyl furo[3,2-c]pyridine-4-carboxylate

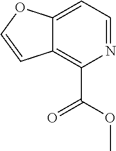

To a solution of 4-chlorofuro[3,2-c]pyridine (15.0 g, 97.7 mmol, 1 eq.) in DMF (150 mL): MeOH (150 mL) was added TEA (49.4 g, 68.1 mL, 488 mmol, 5 eq.) and Pd(dppf)Cl₂ (10 g, 14.7 mmol, 0.15 eq.). The reaction was stirred at 80° C. for 16 hours under CO atmosphere. The resulting solution was diluted with 100 mL of water and extracted with EtOAc (3* 150 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford methyl furo[3,2-c]pyridine-4-carboxylate (8.0 g, yield 46.2%) as a white solid. LC-MS (ESI): mass calcd. for C₉H₇NO₃, 177.0; m/z found, 178.0 [M+H]⁺.

### Step B: furo[3,2-c]pyridin-4-ylmethanol

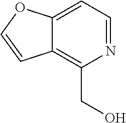 [SMILES: OCc1nccc2occc12]

To a mixture of methyl furo[3,2-c]pyridine-4-carboxylate (8.0 g, 45.2 mmol, 1 eq.) in MeOH (30 mL) was added NaBH₄ (25.6 g, 677 mmol, 15 eq.), and the solution was stirred at 25° C. for 3 hours under N₂ atmosphere. The resulting solution was diluted with 100 mL of water and extracted with EtOAc (3* 100 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford furo[3,2-c]pyridin-4-ylmethanol (4.0 g, yield 59.4%) as a yellow solid. LC-MS (ESI): mass calcd. for C₈H₇NO₂, 149.0; m/z found, 150.0 [M+H]⁺.

### Step C: 4-(((tert-butyldimethylsilyl)oxy)methyl)furo[3,2-c]pyridine

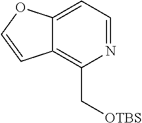 [SMILES: CC(C)(C)[Si](C)(C)OCc1nccc2occc12]

To a stirred mixture of furo[3,2-c]pyridin-4-ylmethanol (4.0 g, 26.8 mmol, 1 eq.) and Imidazole (3.6 g, 53.6 mmol, 2 eq.) in DCM (40 mL) was added TBSCI (6.1 g, 40.2 mmol, 1.5 eq.), the reaction mixture was stirred at 25° C. for 1 hour under N₂ atmosphere. The resulting solution was diluted with 50 mL of water and extracted with DCM (3*50 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 4-(((tert-butyldimethylsilyl)oxy)methyl)furo[3,2-c]pyridine (4.0 g, yield 56.6%) as a yellow solid. LC-MS (ESI): mass calcd. for C₁₄H₂₁NO₂Si, 263.1; m/z found, 264.0 [M+H]⁺.

### Step D: 4-(((tert-butyldimethylsilyl)oxy)methyl)-2-iodofuro[3,2-c]pyridine

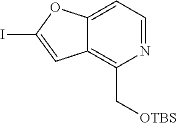 [SMILES: CC(C)(C)[Si](C)(C)OCc1nccc2oc(I)cc12]

To a solution of 4-(((tert-butyldimethylsilyl)oxy)methyl)furo[3,2-c]pyridine (4.0 g, 15.2 mmol, 1 eq.) in THF (30 mL) was added LDA (18.2 mL, 18.2 mmol, 1.2 eq., 1 M in THF) at −78° C. The reaction was stirred at −78° C. for 1 hour under N₂ atmosphere. Then added a mixture of 12 (4.6 g, 18.2 mmol, 1.2 eq.) in THF (3 mL). The reaction was stirred at −78° C. for 1 hour under N₂ atmosphere. LCMS showed the reaction was completed. The mixture was diluted with sat. NH₄Cl and diluted with 60 mL of water and extracted with EtOAc (3*60 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 4-(((tert-butyldimethylsilyl)oxy)methyl)-2-iodofuro[3,2-c]pyridine (2.7 g, yield 45.7%) as a white solid. LC-MS (ESI): mass calcd. for C₁₄H₂₀INO₂Si, 389.0; m/z found, 390.0 [M+H]⁺.

### Step E: (2-iodofuro[3,2-c]pyridin-4-yl)methanol

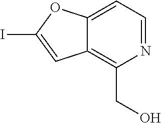 [SMILES: OCc1nccc2oc(I)cc12]

To the solution of 4-(((tert-butyldimethylsilyl)oxy)methyl)-2-iodofuro[3,2-c]pyridine (2.00 g, 5.14 mmol, 1 eq.) in MeCN (25 mL) was added HCl (5 mL) (4M in 1,4-dioxane) at 0° C. and stirred at 0° C. for 2 hours. The resulting solution was adjusted pH=7-9 with aq.

NaHCO₃ and extracted with EtOAc (50 mL) and the layers were separated. The organic layer was washed with brine (20 mL) three times, dried over Na₂SO₄, filtered and concentrated. The residue was purified by flash chromatography (100-200 mesh silica gel, 0%-100% EtOAc in PE) to afford (2-iodofuro[3,2-c]pyridin-4-yl)methanol (1.0 g, yield 71%) as a yellow solid. LC-MS (ESI): mass calcd. for C₈H₆INO₂, 274.9; m/z found, 276.0 [M+H]⁺.

### Step F: 2-(2-iodofuro[3,2-c]pyridin-4-yl)acetonitrile

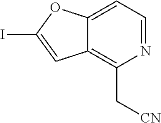 [SMILES: N#CCc1nccc2oc(I)cc12]

To a solution of (2-iodofuro[3,2-c]pyridin-4-yl)methanol (1.0 g, 3.6 mmol, 1 eq.) in THF (10 mL) was added tributylphosphane (1.5 g, 7.2 mmol, 2 eq.) and (E)-diazene-1,2-diylbis(piperidin-1-ylmethanone) (1.8 g, 7.2 mmol, 2 eq.). The mixture was flushed three times with nitrogen, then added 2-hydroxy-2-methylpropanenitrile (464 mg, 5.4 mmol, 1.5 eq.) under N₂ atmosphere. The reaction was stirred at 25° C. for 1 hour. and diluted with 60 mL of water and extracted with EtOAc (3*60 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 2-(2-iodofuro[3,2-c]pyridin-4-yl)acetonitrile (500 mg, yield 48.4%) as a white solid. LC-MS (ESI): mass calcd. for C₉H₅IN₂O, 283.9; m/z found, 285.0 [M+H]⁺.

### Step G: ethyl 4-cyano-4-(2-iodofuro[3,2-c]pyridin-4-yl)butanoate

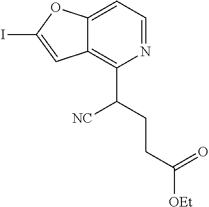 [SMILES: CCOC(=O)CCC(C#N)c1nccc2oc(I)cc12]

To a solution of 2-(2-iodofuro[3,2-c]pyridin-4-yl)acetonitrile (400 mg, 1.4 mmol, 1 eq) and K₂CO₃ (389 mg, 2.8 mmol, 2 eq.) in DMF (15 mL) was added ethyl 3-bromopropanoate (254.9 mg, 1.4 mmol, 1 eq.) and stirred at 50° C. for 1.5 hours. and diluted with 40 mL of water and extracted with EtOAc (3×40 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford ethyl 4-cyano-4-(2-iodofuro[3,2-c]pyridin-4-yl)butanoate (150 mg, yield 28%) as a yellow solid. LC-MS (ESI): mass calcd. for C₁₄H₁₃IN₂O₃, 384.0; m/z found, 385.0 [M+H]⁺.

### Step H: 3-(2-iodofuro[3,2-c]pyridin-4-yl)piperidine-2,6-dione

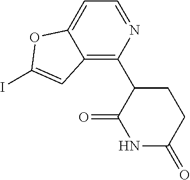 [SMILES: O=C1CCC(c2nccc3oc(I)cc23)C(=O)N1]

To a solution of ethyl 4-cyano-4-(2-iodofuro[3,2-c]pyridin-4-yl)butanoate (150 mg, 390 μmol, 1 eq.) in AcOH (8 mL) was added con. H₂SO₄ (400 μL) with stirring. The mixture was stirred at 100° C. for 2 hours under N₂ atmosphere. The resulting solution was concentrated under reduced pressure and dropped into NaHCO₃with stirring till the pH >7. The resulting solution was diluted with 20 mL of water and extracted with EtOAc (3*20 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 3-(2-iodofuro[3,2-c]pyridin-4-yl)piperidine-2,6-dione (120 mg, yield 86.3%) as a white solid. LC-MS (ESI): mass calcd. for C₁₂H₉IN₂O₃, 356.0; m/z found, 357 [M+H]⁺.

### Step I: tert-butyl (3-(4-(2,6-dioxopiperidin-3-yl)furo[3,2-c]pyridin-2-yl)prop-2-yn-1-yl)carbamate

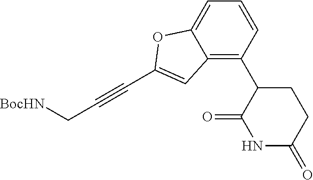 [SMILES: CC(C)(C)OC(=O)NCC#Cc1cc2c(C3CCC(=O)NC3=O)cccc2o1]

To the solution of 3-(2-iodofuro[3,2-c]pyridin-4-yl)piperidine-2,6-dione (50 mg, 140 μmol, 1 eq.) Pd(PPh₃)Cl₂ (20 mg, 28.1 μmol, 0.2 eq.) and CuI (6 mg, 28.1 μmol, 0.2 eq.) and TEA (157 μL, 1.12 mmol, 8 eq.) in DMF (5 mL) was added tert-butyl prop-2-yn-1-ylcarbamate (26 mg, 168 μmol, 1.2 eq.) at 25° C. The mixture was stirred at 80° C. for 2 hours under N₂ atmosphere. The resulting solution was diluted with 20 mL of water and extracted with EtOAc (3*20 mL). The combined organic layers were washed with brine, dried over anhydrous Na₂SO₄ and concentrated under reduced pressure. The residue was purified by flash column chromatography on silica gel (100-200 mesh silica gel, 0-10% MeOH in DCM) to afford tert-butyl (3-(4-(2,6-dioxopiperidin-3-yl)furo[3,2-c]pyridin-2-yl)prop-2-yn-1-yl)carbamate (35.0 mg, yield 65.0%) as a yellow solid. LC-MS (ESI): mass calcd. for C₂₀H₂₁N₃O₅, 383.2; m/z found, 384.2 [M+H]⁺.

### Step J: 3-(2-(3-aminoprop-1-yn-1-yl)furo[3,2-c]pyridin-4-yl)piperidine-2,6-dione

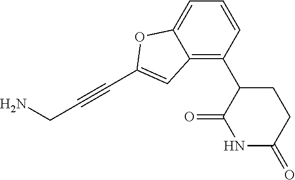 [SMILES: NCC#Cc1cc2c(C3CCC(=O)NC3=O)cccc2o1]

To a solution of tert-butyl (3-(4-(2,6-dioxopiperidin-3-yl)furo[3,2-c]pyridin-2-yl)prop-2-yn-1-yl)carbamate (35 mg, 91.3 μmol, 1 eq.) in DCM (3 mL) were added TFA (0.6 mL), and the reaction was stirred at 25° C. for 30 mins. The reaction was monitored by LCMS and concentrated under reduced pressure to afford crude 3-(2-(3-aminoprop-1-yn-1-yl)furo[3,2-c]pyridin-4-yl)piperidine-2,6-dione (25 mg, crude) as a brown oil. LC-MS (ESI): mass calcd. for C₁₅H₁₃N₃O₃, 283.1; m/z found, 284.1 [M+H]⁺.

### Intermediate 13. 5-(5-((1,3-dimethyl-2-oxo-7-(tetrahydro-2H-pyran-4-yl)-2,3-dihydro-1H-benzo[d]imidazol-5-yl)(methyl)amino)pyrimidin-2-yl)-3-methylpicolinic acid

### Step A: tert-butyl (2-bromopyrimidin-5-yl)(methyl)carbamate

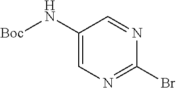 [SMILES: CC(C)(C)OC(=O)Nc1cnc(Br)nc1]

To a solution of tert-butyl (2-bromopyrimidin-5-yl)carbamate (8 g, 30 mmol, 1.0 eq.) in DMF (80 mL) was added NaH (1.5 g, 60 mmol, 2.0 eq.) at 0° C. The mixture was then stirred at 0° C. for 30 mins under N₂. Mel (4 mL, 6 mmol, 2.0 eq.) was added at 0° C. The mixture was then stirred at 25° C. for 1 hour under N₂. The resulting solution was diluted with 500 mL of H₂O and extracted with EtOAc (200 mL×3). The combined organic layers were washed with brine, dried over Na₂SO₄, and concentrated under reduced pressure. The residue was purified by flash column chromatography on silica gel (100-200 mesh silica gel, 0-30% EtOAc in PE) to afford tert-butyl (2-bromopyrimidin-5-yl)(methyl)carbamate (5 g, yield 60%) as a white solid. LC-MS (ESI): mass calcd. for C₁₀H₁₄BrN₃O₂, 287.0/289.0; m/z found, 288.0/290.0 [M+H]⁺. Step B: methyl 5-(8-(1,3-dimethyl-2-oxo-7-(((trifluoromethyl)sulfonyl)oxy)-1,2-dihydroquinolin-5-yl)isoquinolin-3-yl)picolinate

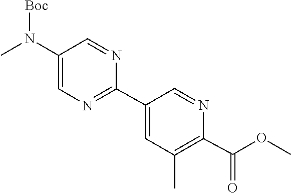

To the solution of methyl 3-methyl-5-(4,4,5,5-tetramethyl-1,3,2-dioxaborolan-2-yl)picolinate (4 g, 14.6 mmol, 1.5 eq.), tert-butyl (2-bromopyrimidin-5-yl)(methyl)carbamate (2.8 g, 9.72 mmol, 1.0 eq.) and K₂CO₃ (4 g, 29 mmol, 3.0 eq.) in 1,4-Dioxane: H₂O(100 mL, 10:1) was added 1,1′-Bis(diphenylphosphino)ferrocene-palladium(II) dichloride (711 mg, 972 μmol, 0.1 eq.) at r.t. The mixture was stirred at 100° C. for 1.5 hours under N₂ atmosphere. The resulting solution was diluted with 100 mL of H₂O and extracted with EtOAc (100 mL×3). The combined organic layers were washed with brine, dried over Na₂SO₄, and concentrated under reduced pressure. The residue was purified by flash column chromatography on silica gel (100-200 mesh silica gel, 0-60% EtOAc in PE) to afford methyl 5-(5-((tert-butoxycarbonyl)(methyl)amino)pyrimidin-2-yl)-3-methylpicolinate (3.3 g, yield 95%) as a brown soild.

LC-MS (ESI): mass calcd. for C₁₈H₂₂N₄O₄, 358.1; m/z found, 359.1 [M+H]⁺.

### Step C: methyl 3-methyl-5-(5-(methylamino)pyrimidin-2-yl)picolinate

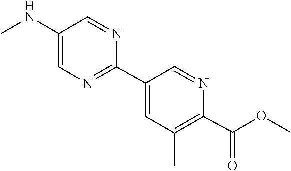

To a solution of methyl 5-(5-((tert-butoxycarbonyl)(methyl)amino)pyrimidin-2-yl)-3-methylpicolinate (2.1 g, 5.8 mmol, 1.0 eq.) in DCM (20 mL) was added TFA (4 mL) stirred at 25° C. for 3 hours. The resulting solution was dried over anhydrous sodium sulfate and concentrated under reduced pressure to afford methyl 3-methyl-5-(5-(methylamino)pyrimidin-2-yl)picolinate (1.4 g, yield 92%) as a yellow solid.

LC-MS (ESI): mass calcd. for C₁₃H₁₄N₄O₂, 258.1; m/z found, 259.1 [M+H]⁺.

### Step D: methyl 5-(5-((1,3-dimethyl-2-oxo-7-(tetrahydro-2H-pyran-4-yl)-2,3-dihydro-1H-benzo[d]imidazol-5-yl)(methyl)amino)pyrimidin-2-yl)-3-methylpicolinate

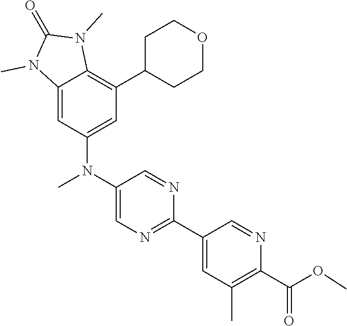

To a solution of 1,3-dimethyl-2-oxo-7-(tetrahydro-2H-pyran-4-yl)-2,3-dihydro-1H-benzo[d]imidazol-5-yl trifluoromethanesulfonate (450 mg, 1.1 mmol, 1.0 eq.) methyl 3-methyl-5-(5-(methylamino)pyrimidin-2-yl)picolinate (294 mg, 1.1 mmol, 1.0 eq.), Cs₂CO₃ (1.1 g, 3.4 mmol, 3.0 eq.) and Pd₂(dba)₃ (104 mg, 114 μmol, 0.1 eq.) in Toluene (8 mL) was added 2-(Dicyclohexylphosphanyl)-2′,4′,6′-tris(isopropyl)biphenyl (108 mg, 228 μmol, 0.2 eq.) at 25° C. The mixture was stirred at 100° C. for 5 hours under N₂ atmosphere. The mixture was diluted water and extracted with EtOAC (30 mL×3). The combined organic layers were washed with brine (30 mL), dried over Na₂SO₄, filtered and concentrated. The residue was purified by flash chromatography (100-200 mesh silica gel, 0-30% EtOAc in PE) to afford methyl 5-(5-((1,3-dimethyl-2-oxo-7-(tetrahydro-2H-pyran-4-yl)-2,3-dihydro-1H-benzo[d]imidazol-5-yl)(methyl)amino)pyrimidin-2-yl)-3-methylpicolinate (210 mg, yield 36%) as a yellow solid.

LC-MS (ESI): mass calcd. for C₂₇H₃₀N₆O₄, 502.2; m/z found, 503.2 [M+H]⁺. Step E: 5-(5-((1,3-dimethyl-2-oxo-7-(tetrahydro-2H-pyran-4-yl)-2,3-dihydro-1H-benzo[d]imidazol-5-yl)(methyl)amino)pyrimidin-2-yl)-3-methylpicolinic acid

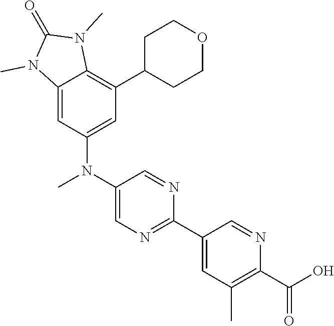 [SMILES: Cc1cc(-c2ncc(N(C)c3cc(C4CCOCC4)c4c(c3)n(C)c(=O)n4C)cn2)cnc1C(=O)O]

To a solution of methyl 5-(5-((1,3-dimethyl-2-oxo-7-(tetrahydro-2H-pyran-4-yl)-2,3-dihydro-1H-benzo[d]imidazol-5-yl)(methyl)amino)pyrimidin-2-yl)-3-methylpicolinate (230 mg, 457 μmol, 1.0 eq.) in THF:H₂O=1:1 (3 mL) was added Lithium hydroxide monohydrate (38 mg, 915 μmol, 2.0 eq.) at 25° C. The mixture was stirred at 25° C. for 30 mins under N₂ atmosphere.

The resulting solution was concentrated under reduced pressure to afford 5-(5-((1,3-dimethyl-2-oxo-7-(tetrahydro-2H-pyran-4-yl)-2,3-dihydro-1H-benzo[d]imidazol-5-yl)(methyl)amino)pyrimidin-2-yl)-3-methylpicolinic acid (226 mg, crude) as a yellow solid.

LC-MS (ESI): mass calcd. for C₂₆H₂₈N₆O₄, 488.2; m/z found, 489.2 [M+H]⁺.

### Intermediate 14. 5-(8-(4-(3-hydroxyazetidin-1-yl)-1,3-dimethyl-2-oxo-2,3-dihydro-1H-imidazo[4,5-c]pyridin-6-yl)isoquinolin-3-yl)picolinic acid

### Step A: 6-chloro-4-(3-hydroxyazetidin-1-yl)-1,3-dimethyl-1,3-dihydro-2H-imidazo[4,5-c]pyridin-2-one

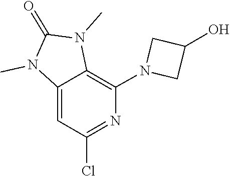 [SMILES: Cn1c(=O)n(C)c2c(N3CC(O)C3)nc(Cl)cc21]

To a mixture of azetidin-3-ol (236 mg, 3.2 mmol, 1.5 eq.) and 4,6-dichloro-1,3-dimethyl-1,3-dihydro-2H-imidazo[4,5-c]pyridin-2-one (500 mg, 2.1 mmol, 1 eq.) in DMSO (6 mL) was added DABCO (48 mg, 431 μmol, 0.2 eq.) and K₂CO₃ (893 mg, 6.5 mmol, 3 eq.), and the solution was stirred at 120° C. for 12 hours. The resulting solution was diluted with 50 ml of water and extracted with EtOAc (3*60 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 6-chloro-4-(3-hydroxyazetidin-1-yl)-1,3-dimethyl-1,3-dihydro-2H-imidazo[4,5-c]pyridin-2-one (310 mg, yield 53.5%) as a white solid.

¹H NMR (400 MHz, DMSO-d₆) δ 6.94 (s, 1H), 5.62 (d, J=6.3 Hz, 1H), 4.57-4.47 (m, 1H), 4.28-4.21 (m, 2H), 3.86 (dd, J=9.0, 5.2 Hz, 2H), 3.43 (s, 3H), 3.28 (s, 3H).

### Step B: methyl 5-(8-(4-(3-hydroxyazetidin-1-yl)-1,3-dimethyl-2-oxo-2,3-dihydro-1H-imidazo[4,5-c]pyridin-6-yl)isoquinolin-3-yl)picolinate

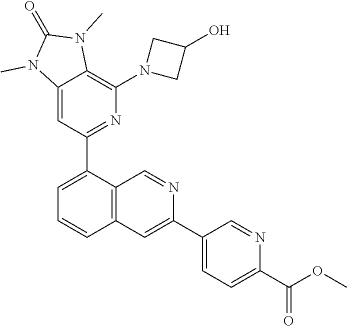

To a mixture of methyl 5-(8-(4,4,5,5-tetramethyl-1,3,2-dioxaborolan-2-yl)isoquinolin-3-yl)picolinate (240 mg, 614 μmol, 1.1 eq,), 6-chloro-4-(3-hydroxyazetidin-1-yl)-1,3-dimethyl-1,3-dihydro-2H-imidazo[4,5-c]pyridin-2-one (150 mg, 558 μmol, 1 eq.) and K₂CO₃ (231 mg, 1.67 mmol, 3 eq.) in DMF (3 mL) and was added X-Phos Palladacycle (43.9 mg, 55.8 μmol, 0.1 eq.), the reaction at 100° C. for 1 hour. The resulting solution was diluted with 50 ml of water and extracted with EtOAc (3*30 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford methyl 5-(8-(4-(3-hydroxyazetidin-1-yl)-1,3-dimethyl-2-oxo-2,3-dihydro-1H-imidazo[4,5-c]pyridin-6-yl)isoquinolin-3-yl)picolinate (60.0 mg, yield 21.6%) as a yellow solid. LC-MS (ESI): mass calcd. for C₂₇H₂₄N₆O₄, 496.2; m/z found, 497.2 [M+H]⁺.

### Step C: 5-(8-(4-(3-hydroxyazetidin-1-yl)-1,3-dimethyl-2-oxo-2,3-dihydro-1H-imidazo[4,5-c]pyridin-6-yl)isoquinolin-3-yl)picolinic acid

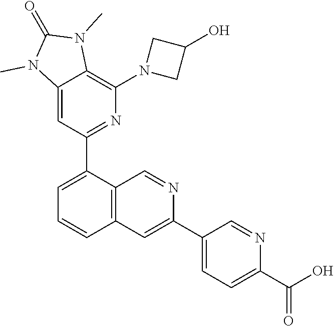 [SMILES: Cn1c(=O)n(C)c2c(N3CC(O)C3)nc(-c3cccc4cc(-c5ccc(C(=O)O)nc5)ncc34)cc21]

To the solution of methyl 5-(8-(4-(3-hydroxyazetidin-1-yl)-1,3-dimethyl-2-oxo-2,3-dihydro-1H-imidazo[4,5-c]pyridin-6-yl)isoquinolin-3-yl)picolinate (20 mg, 40.3 μmol, 1 eq.) in THF (3 mL) was added the solution of lithium hydroxide (2 mg, 80.6 μmol, 2 eq.) in H₂O(600 μL) and stirred at 25° C. for 30 mins. The resulting solution was diluted with 50 ml of water and extracted with EtOAc (3*20 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 5-(8-(4-(3-hydroxyazetidin-1-yl)-1,3-dimethyl-2-oxo-2,3-dihydro-1H-imidazo[4,5-c]pyridin-6-yl)isoquinolin-3-yl)picolinic acid (20 mg, crude) as a yellow solid. LC-MS (ESI): mass calcd. for C₂₆H₂₂N₆O₄, 482.2; m/z found, 483.2 [M+H]⁺.

### Intermediate 15. 3-(5-(3-aminoprop-1-yn-1-yl)pyrazolo[1,5-a]pyridin-3-yl)piperidine-2,6-dione

### Step A: 3-(2,6-bis(benzyloxy)pyridin-3-yl)-5-bromopyrazolo[1,5-a]pyridine

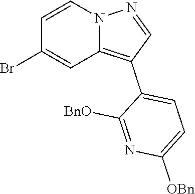 [SMILES: Brc1ccn2ncc(-c3ccc(OCc4ccccc4)nc3OCc3ccccc3)c2c1]

To a solution of 5-bromo-3-iodopyrazolo[1,5-a]pyridine (2.0 g, 6.2 mmol, 1.0 eq.) in 1,4-Dioxane (20 mL) and H₂O(2 mL) was added 2,6-bis(benzyloxy)-3-(4,4,5,5-tetramethyl-1,3,2-dioxaborolan-2-yl)pyridine (2.8 g, 6.8 mmol, 1.1 eq.), Na₂CO₃ (2.0 g, 18.6 mmol, 3.0 eq.) and Pd(dppf)Cl₂ (453 mg, 619 μmol, 0.1 eq.) under N₂. The reaction mixture was stirred at 60° C. for 2 hours. The mixture was diluted with water and extracted with EtOAc (50 mL×3). The combined organic layers were washed with brine, dried over Na₂SO₄ and concentrated under reduced pressure. The residue was purified by column chromatography on silica gel eluted with EtOAc: PE (100:1-5:1) to give 3-(2,6-bis(benzyloxy)pyridin-3-yl)-5-bromopyrazolo[1,5-a]pyridine (1.0 g, 33% yield) as a yellow solid.

LC-MS (ESI): mass calcd. for C₂₆H₂₀BrN₃O₂, 485.07; m/z found, 486.07/488.07 [M+H]+.

### Step B: 3-(2,6-bis(benzyloxy)pyridin-3-yl)-5-(4,4,5,5-tetramethyl-1,3,2-dioxaborolan-2-yl)pyrazolo[1,5-a]pyridine

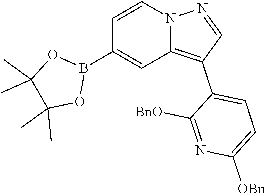 [SMILES: CC1(C)OB(c2ccn3ncc(-c4ccc(OCc5ccccc5)nc4OCc4ccccc4)c3c2)OC1(C)C]

To a solution of 3-(2,6-bis(benzyloxy)pyridin-3-yl)-5-bromopyrazolo[1,5-a]pyridine (1 g, 2.1 mmol, 1.0 eq.) in 1,4-Dioxane (15 mL) was added 4,4,4′,4′,5,5,5′,5′-octamethyl-2,2′-bi(1,3,2-dioxaborolane) (627 mg, 2.5 mmol, 1.2 eq.), Potassium acetate (605 mg, 6.2 mmol, 3.0 eq.) and Pd(dppf)Cl₂ (150 mg, 206 μmol, 0.1 eq.) under N₂. The reaction mixture was stirred at 100° C. for 3 hours. The resulting solution was diluted with 20 ml of water and extracted with EtOAc (20 mL×3). The combined organic layers were washed with brine (20 mL×3), dried over Na₂SO₄ and concentrated under reduced pressure. The residue was purified by column chromatography on silica gel eluted with EtOAc: PE (100:1˜10:1) to give 3-(2,6-bis(benzyloxy)pyridin-3-yl)-5-(4,4,5,5-tetramethyl-1,3,2-dioxaborolan-2-yl)pyrazolo[1,5-a]pyridine (800 mg, 73% yield) as a white solid.

LC-MS (ESI): mass calcd. for C₃₂H₃₂BN₃O₄, 533.25; m/z found, 534.25 [M+H]⁺.

### Step C: 3-(2,6-bis(benzyloxy)pyridin-3-yl)pyrazolo[1,5-a]pyridin-5-ol

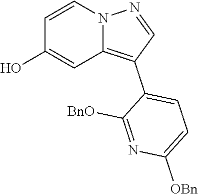 [SMILES: Oc1ccn2ncc(-c3ccc(OCc4ccccc4)nc3OCc3ccccc3)c2c1]

To a solution of 3-(2,6-bis(benzyloxy)pyridin-3-yl)-5-(4,4,5,5-tetramethyl-1,3,2-dioxaborolan-2-yl)pyrazolo[1,5-a]pyridine (800 mg, 1.5 mmol, 1.0 eq.) in Acetone (10 mL) and H₂O(5 mL)was added Oxone (922 mg, 1.5 mmol, 1.0 eq.). The reaction mixture was stirred at 30° C. for 1 hour. The resulting solution was filtered and washed with water. The filter cake was purified by column chromatography on silica gel eluted with EtOAc: PE (100:1˜1:1) to give 3-(2,6-bis(benzyloxy)pyridin-3-yl)pyrazolo[1,5-a]pyridin-5-ol (450 mg, 71% yield) as a white solid.

LC-MS (ESI): mass calcd. for C₂₆H₂₁N₃O₃, 423.16; m/z found, 424.16 [M+H]⁺.

### Step D: 3-(5-hydroxypyrazolo[1,5-a]pyridin-3-yl)piperidine-2,6-dione

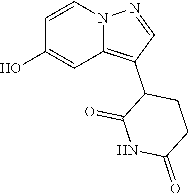 [SMILES: O=C1CCC(c2cnn3ccc(O)cc23)C(=O)N1]

To a solution of 3-(2,6-bis(benzyloxy)pyridin-3-yl)pyrazolo[1,5-a]pyridin-5-ol (300 mg, 708 μmol, 1.0 eq.) in EtOH (30 mL) was added added Pd/C (120 mg, w.t.=10%). The mixture was degassed with H₂ for 3 times and stirred at 60° C. using a H₂ balloon for 4 hours. The resulting solution was filtered through a Celite pad and filtrate was concentrated under reduced pressure. The residue was purified with flash column chromatography on silica gel eluted with EtOAc: PE (100:1˜10:3) to afford 3-(5-hydroxypyrazolo[1,5-a]pyridin-3-yl)piperidine-2,6-dione (100 mg, 57.6% yield) as a white solid.

LC-MS (ESI): mass calcd. for C₁₂H₁₁N₃O₃, 245.08; m/z found, 246.08 [M+H]⁺.

### Step E: 3-(2,6-dioxopiperidin-3-yl)pyrazolo[1,5-a]pyridin-5-yl trifluoromethanesulfonate

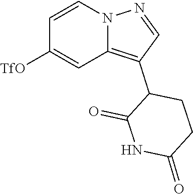 [SMILES: O=C1CCC(c2cnn3ccc(OS(=O)(=O)C(F)(F)F)cc23)C(=O)N1]

To a solution of 3-(5-hydroxypyrazolo[1,5-a]pyridin-3-yl)piperidine-2,6-dione (80 mg, 326 μmol, 1.0 eq.) in DCM (10 mL) was added TEA (91 μL, 652 μmol, 2.0 eq.) and Phenyl triflimide (175 mg, 489 μmol, 1.5 eq.). The reaction mixture was stirred at 30° C. for 1 hour. The resulting solution was diluted with 20 ml of water and extracted with DCM (20 mL×3). The combined organic layers were washed with brine (20 mL×3), dried over Na₂SO₄ and concentrated under reduced pressure. The residue was purified by column chromatography on silica gel eluted with EtOAc: PE (100:1˜100:70) to give 3-(2,6-dioxopiperidin-3-yl)pyrazolo[1,5-a]pyridin-5-yl trifluoromethanesulfonate (50 mg, 40% yield) as a yellow oil.

LC-MS (ESI): mass calcd. for C₁₃H₁₀F3N₃₀₅S, 377.03; m/z found, 378.03 [M+H]⁺.

### Step F: tert-butyl (3-(3-(2,6-dioxopiperidin-3-yl)pyrazolo[1,5-a]pyridin-5-yl)prop-2-yn-1-yl)carbamate

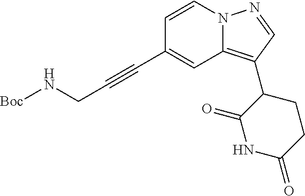 [SMILES: CC(C)(C)OC(=O)NCC#Cc1ccn2ncc(C3CCC(=O)NC3=O)c2c1]

To a solution of 3-(2,6-dioxopiperidin-3-yl)pyrazolo[1,5-a]pyridin-5-yl trifluoromethanesulfonate (30.0 mg, 80 μmol, 1.0 eq.) in DMF (2 mL) and TEA (0.5 mL, 800 μmol, 10.0 eq.) was added CuI (1.5 mg, 8 μmol, 0.1 eq.) and Pd(PPh₃)₂C₁₂ (5.58 mg, 8 μmol, 0.1 eq.) and tert-butyl prop-2-yn-1-ylcarbamate (37 mg, 3 240 μmol, 3.0 eq.) under N₂. The reaction mixture was stirred at 30° C. for 2 hours. The resulting solution was diluted with 20 ml of water and extracted with EtOAc (20 mL×3). The combined organic layers were washed with brine (20 mL×3), dried over Na₂SO₄ and concentrated under reduced pressure. The residue was purified by column chromatography on silica gel eluted with EtOAc: PE (100:1-100:60) to give tert-butyl (3-(3-(2,6-dioxopiperidin-3-yl)pyrazolo[1,5-a]pyridin-5-yl)prop-2-yn-1-yl)carbamate (20 mg, 66% yield) as a yellow solid.

LC-MS (ESI): mass calcd. for C₂₀H₂₂N₄O₄, 382.16; m/z found, 383.16 [M+H]⁺.

### Step G: 3-(5-(3-aminoprop-1-yn-1-yl)pyrazolo[1,5-a]pyridin-3-yl)piperidine-2,6-dione

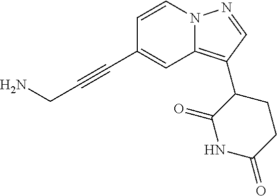 [SMILES: NCC#Cc1ccn2ncc(C3CCC(=O)NC3=O)c2c1]

To a solution of tert-butyl (3-(3-(2,6-dioxopiperidin-3-yl)benzofuran-5-yl)prop-2-yn-1-yl)carbamate (20 mg, 52 μmol, 1.0 eq.) in DCM (3 mL) was added HCl (1 mL, 4 M in 1.4-dioxane). The reaction mixture was stirred at 30° C. for 1 hour. The resulting solution was concentrated under reduced pressure to give 3-(5-(3-aminoprop-1-yn-1-yl)benzofuran-3-yl)piperidine-2,6-dione (20 mg, crude) as a brown solid.

LC-MS (ESI): mass calcd. for C₁₅H₁₄N₄O₂, 282.11; m/z found, 283.11 [M+H]⁺.

### Intermediate 16. 5-(6-fluoro-4-(7-isopropyl-1,3-dimethyl-2-oxo-2,3-dihydro-1H-benzo[d]imidazol-5-yl)-2,2-dimethyl-2H-chromen-7-yl)picolinic acid

### Step A: 7-bromo-6-fluoro-2,2-dimethylchroman-4-one

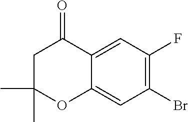

To a solution of propan-2-one (4 g, 68.7 mmol, 4.0 eq.) in THF (40 mL) was added 1-(4-bromo-5-fluoro-2-hydroxyphenyl) ethan-1-one (4 g, 17.2 mmol, 1.0 eq.), DIEA (3 mL, 17.2 mmol, 1.0 eq.) and Pyrrolidine (1.3 mL, 15.4 mmol, 0.9 eq.), the solution was stirred at 70° C. for 16 hours to give brown solution. TLC showed the reaction was completed. The resulting solution was diluted with 100 mL of water and extracted with EtOAc (3×100 mL). The combined organic layers were washed with brine (100 mL), dried over Na₂SO₄, and concentrated under reduced pressure. The residue was purified by flash chromatography (100-200 mesh silica gel, 0% ˜30% EtOAc in PE) to afford 7-bromo-6-fluoro-2,2-dimethylchroman-4-one (2.4 g, yield 49%) as a yellow solid. The product was no Ms signal. ¹H NMR (400 MHz, DMSO-d₆) δ 7.56 (d, J=8.3 Hz, 1H), 7.46 (d, J=5.5 Hz, 1H), 2.84 (s, 2H), 1.39 (s, 7H).

### Step B: methyl 5-(6-fluoro-2,2-dimethyl-4-oxochroman-7-yl) picolinate

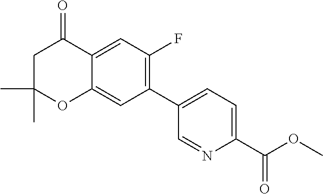

To a solution of methyl 5-(4,4,5,5-tetramethyl-1,3,2-dioxaborolan-2-yl)picolinate (3.5 g, 13.2 mmol, 1.5 eq.) in 1,4-Dioxane (30 mL) and Water (1.5 mL) was added 7-bromo-6-fluoro-2,2-dimethylchroman-4-one (2.4 g, 8.8 mmol, 1.0 eq.), K₂CO₃ (3.6 g, 26 mmol, 3.0 eq.) and Pd(dppf)Cl₂ (643 mg, 879 μmol, 0.1 eq.) under N₂. The solution was stirred at 100° C. for 2 hours to give black solution. LCMS showed the reaction was completed. The resulting solution was diluted with 100 mL of water and extracted with EtOAc (3×100 mL). The combined organic layers were washed with brine (100 mL), dried over Na₂SO₄, and concentrated under reduced pressure. The residue was purified by flash chromatography (100-200 mesh silica gel, 0% ˜100% EtOAc in PE) to afford methyl 5-(6-fluoro-2,2-dimethyl-4-oxochroman-7-yl) picolinate (2.5 g, yield 82%) as a yellow solid. LC-MS (ESI): mass calcd. for C₁₈H₁₆FNO4, 330.2; m/z found, 331.2 [M+H]⁺.

### Step C: methyl 5-(6-fluoro-2,2-dimethyl-4-(((trifluoromethyl)sulfonyl)oxy)-2H-chromen-7-yl)picolinate

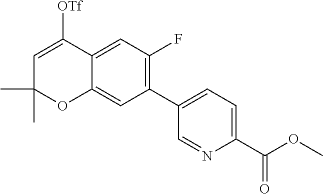

To a solution of methyl 5-(6-fluoro-2,2-dimethyl-4-oxochroman-7-yl)picolinate (800 mg, 2.4 mmol, 1.0 eq.) in THF (10 mL) was added LiHMDS (0.49 mL, 4.9 mmol, 1M in THF, 2.0 eq.), the solution was stirred at −78° C. for 30 mins, then Tf₂O (1.3 mL, 7.3 mmol,1.677 g/ml, 3.0 eq.) was added to the above solution and stirred at −78° C. for 30 mins to give yellow solution. LCMS showed the reaction was completed. The solution was quenched with sat. NH₄C₁ solution, extracted with EtOAc (50 mL) and the layers were separated. The organic layer was washed with brine (50 mL), dried over Na₂SO₄, filtered and concentrated. The residue was purified by flash chromatography (100-200 mesh silica gel, 0% ˜30% EtOAC in PE) to afford methyl 5-(6-fluoro-2,2-dimethyl-4-(((trifluoromethyl)sulfonyl)oxy)-2H-chromen-7-yl)picolinate (400 mg, yield 18%) as a yellow solid. LC-MS (ESI): mass calcd. for C₁₉H₁₅F₄NO₆S, 461.3; m/z found, 462.3 [M+H]⁺.

### Step D: methyl 5-(6-fluoro-4-(7-isopropyl-1,3-dimethyl-2-oxo-2,3-dihydro-1H-benzo[d]imidazol-5-yl)-2,2-dimethyl-2H-chromen-7-yl)picolinate

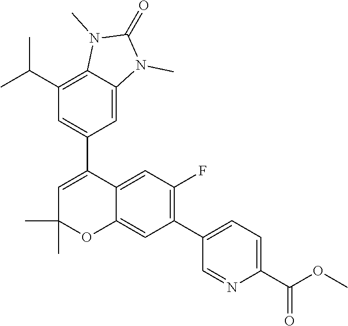 [SMILES: COC(=O)c1ccc(-c2cc3c(cc2F)C(c2cc(C(C)C)c4c(c2)n(C)c(=O)n4C)=CC(C)(C)O3)cn1]

To a solution of 4-isopropyl-1,3-dimethyl-6-(4,4,5,5-tetramethyl-1,3,2-dioxaborolan-2-yl)-1,3-dihydro-2H-benzo[d]imidazol-2-one (315 mg, 954 μmol, 1.1 eq.) in 1,4-Dioxane (10 mL) and Water (0.5 mL) was added methyl 5-(6-fluoro-2,2-dimethyl-4-(((trifluoromethyl)sulfonyl)oxy)-2H-chromen-7-yl)picolinate (400 mg, 867 μmol, 1 0.0 eq.), K₃PO₄ (552 mg, 2.6 mmol, 3.0 eq.) and Pd(dppf)Cl₂ (63.4 mg, 86.7 μmol, 0.1 eq.) under N₂. The solution was stirred at 100° C. for 2 hours to give black solution. LCMS showed the reaction was completed. The resulting solution was diluted with 20 mL of water and extracted with EtOAc (3×30 mL). The combined organic layers were washed with brine (30 mL), dried over Na₂SO₄, and concentrated under reduced pressure. The residue was purified by flash chromatography (100-200 mesh silica gel, 0% ˜100% EtOAC in PE) to afford methyl 5-(6-fluoro-4-(7-isopropyl-1,3-dimethyl-2-oxo-2,3-dihydro-1H-benzo[d]imidazol-5-yl)-2,2-dimethyl-2H-chromen-7yl) picolinate (150 mg, yield 32%) as a yellow solid. LC-MS (ESI): mass calcd. for C₃₀H₃₀FN₃O₄, 515.6; m/z found, 516.6 [M+H]⁺.

### Step E: 5-(6-fluoro-4-(7-isopropyl-1,3-dimethyl-2-oxo-2,3-dihydro-1H-benzo[d]imidazol-5-yl)-2,2-dimethyl-2H-chromen-7-yl)picolinic acid

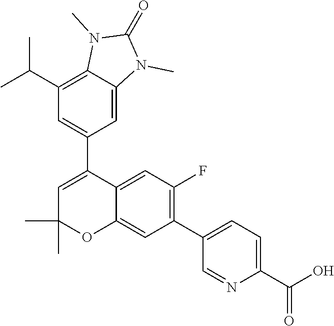 [SMILES: CC(C)c1cc(C2=CC(C)(C)Oc3cc(-c4ccc(C(=O)O)nc4)c(F)cc32)cc2c1n(C)c(=O)n2C]

To a solution of methyl 5-(6-fluoro-4-(7-isopropyl-1,3-dimethyl-2-oxo-2,3-dihydro-1H-benzo[d]imidazol-5-yl)-2,2-dimethyl-2H-chromen-7-yl)picolinate (50 mg, 97 μmol, 1.0 eq.) in THF (5 mL) and Water (1 mL) was added LiGH (8.1 mg, 194 μmol, 2.0 eq.), the solution was stirred at 25° C. for 30 mins to give yellow solution. LCMS showed the reaction was completed. The solution was concentrated to afford 5-(6-fluoro-4-(7-isopropyl-1,3-dimethyl-2-oxo-2,3-dihydro-1H-benzo[d]imidazol-5-yl)-2,2-dimethyl-2H-chromen-7-yl)picolinic acid (50 mg, Li salt, crude) as a yellow solid. LC-MS (ESI): mass calcd. for C₂₉H₂₈FN₃O₄, 501.6; m/z found, 502.6 [M+H]⁺.

### Intermediate 17. 5-(5-(1,3-dimethyl-2-oxo-7-(tetrahydro-2H-pyran-4-yl-3,4-d2)-1,2-dihydroquinolin-5-yl)-5,6,7,8-tetrahydropyrido[3,2-d]pyrimidin-2-yl)picolinic acid

### Step A: 7-(3,6-dihydro-2H-pyran-4-yl)-5-methoxy-1,3-dimethylquinolin-2(1H)-one

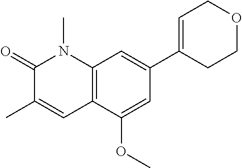 [SMILES: COc1cc(C2=CCOCC2)cc2c1cc(C)c(=O)n2C]

A mixture of 7-bromo-5-methoxy-1,3-dimethylquinolin-2(1H)-one (600 mg, 2.1 mmol, 1.0 eq.), 2-(3,6-dihydro-2H-pyran-4-yl)-4,4,5,5-tetramethyl-1,3,2-dioxa borolane (1.3 g, 6.3 mmol, 3.0 eq.), Pd(dppf)Cl₂ (233 mg, 319 μmol, 0.15 eq.) and Na₂CO₃ (676 mg, 6.3 mmol, 3.0 eq.) in 1,4-Dioxane (10 mL) and H₂O(1 mL) was stirred at 100° C. for 3 hours under N₂. The resulting solution was diluted with 50 mL of water and extracted with EtOAc (20 mL×3). The combined organic layers were washed with brine (50 mL), dried over Na₂SO₄ and concentrated under reduced pressure. The residue was purified by flash column chromatography on silica gel (100-200 mesh silica gel, 0-50% EtOAc and 10% DCM in PE) to give 7-(3,6-dihydro-2H-pyran-4-yl)-5-methoxy-1,3-dimethylquinolin-2(1H)-one (400 mg, 66% yield) as a yellow solid. LC-MS (ESI): mass calcd. for C₁₇H₁₉NO₃, 285.14; m/z found, 286 [M+H]⁺.

### Step B: 5-methoxy-1,3-dimethyl-7-(tetrahydro-2H-pyran-4-yl-3,4-d2)quinolin-2(1H)-one

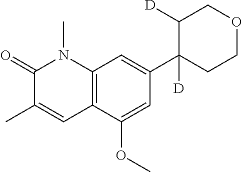 [SMILES: [2H]C1COCCC1([2H])c1cc(OC)c2cc(C)c(=O)n(C)c2c1]

To a solution of 7-(3,6-dihydro-2H-pyran-4-yl)-5-methoxy-1,3-dimethylquinolin-2(1H)-one (1.3 g, 4.5 mmol, 1.0 eq.) in CD30D (100 mL) and Tetrahydrofuran-D8 (10 mL) was stirred at 50° C. for 5 hours under D2. The resulting solution was filtered by celite pad to give 5-methoxy-1,3-dimethyl-7-(tetrahydro-2H-pyran-4-yl-3,4-d2)quinolin-2(1H)-one (1.2 g, 91% yield) as a white solid. LC-MS (ESI): mass calcd. for C₁₇H₁₉D₂NO₃, 289.16; m/z found, 290 [M+H]⁺.

### Step C: 1,3-dimethyl-2-oxo-7-(tetrahydro-2H-pyran-4-yl-3,4-d2)-1,2-dihydroquinolin -5-yl trifluoromethanesulfonate

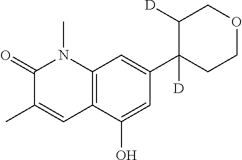 [SMILES: [2H]C1COCCC1([2H])c1cc(O)c2cc(C)c(=O)n(C)c2c1]

A mixture of 5-methoxy-1,3-dimethyl-7-(tetrahydro-2H-pyran-4-yl-3,4-d2)quinolin-2(1H)-one (450 mg, 1.6 mmol, 1.0 eq.) and sodium ethanethiolate (1.3 g, 15.6 mmol, 10.0 eq.) in DMF (10 mL) was stirred at 110° C. for 2 hours under N₂. The resulting solution was removed under reduced pressure. The residue was purified by flash column chromatography on silica gel (100-200 mesh silica gel, 0-80% EtOAc in PE) to give 5-hydroxy-1,3-dimethyl-7-(tetrahydro-2H-pyran-4-yl-3,4-d2)quinolin-2(1H)-one (300 mg, 70% yield) as a yellow solid.

LC-MS (ESI): mass calcd. for C₁₆H₁₇D₂NO₃, 275.15; m/z found, 276 [M+H]⁺.

### Step D: 1,3-dimethyl-2-oxo-7-(tetrahydro-2H-pyran-4-yl-3,4-d2)-1,2-dihydroquinolin -5-yl trifluoromethanesulfonate

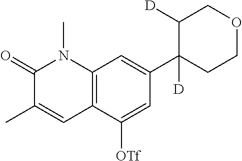 [SMILES: [2H]C1COCCC1([2H])c1cc(OS(=O)(=O)C(F)(F)F)c2cc(C)c(=O)n(C)c2c1]

To a solution of 5-hydroxy-1,3-dimethyl-7-(tetrahydro-2H-pyran-4-yl-3,4-d2)quinolin-2(1H)-one (879 mg, 3.2 mmol, 1.0 eq), Pyridine (1.0 mL, 12.8 mmol, 4.0 eq.) in DCM (15.0 mL) was added trifluoromethanesulfonic anhydride (1.8 g, 6.4 mmol 2.0 eq.) at 0° C.

The mixture was stirred at 0° C. for 2 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-100% EtOAc in PE) to give 1,3-dimethyl-2-oxo-7-(tetrahydro-2H-pyran-4-yl-3,4-d2)-1,2-dihydroquinolin-5-yl trifluoromethanesulfonate (900 mg, 69% yield) as a white solid. LC-MS (ESI): mass calcd. for C₁₇H₁₆D₂F₃NO₅S, 407.10; m/z found, 408 [M+H]⁺.

### Step E: methyl 5-(5-(1,3-dimethyl-2-oxo-7-(tetrahydro-2H-pyran-4-yl-3,4-d2)-1,2-dihydroquinolin-5-yl)-5,6,7,8-tetrahydropyrido[3,2-d]pyrimidin-2-yl)picolinate

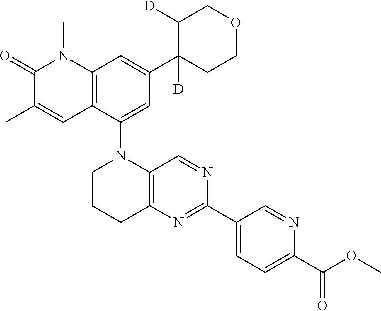

A mixture of 1,3-dimethyl-2-oxo-7-(tetrahydro-2H-pyran-4-yl-3,4-d2)-1,2-dihydroquinolin-5-yl trifluoromethanesulfonate (75 mg, 184 μmol, 1.0 eq.), methyl 5-(5,6,7,8-tetrahydropyrido[3,2-d]pyrimidin-2-yl)picolinate (50 mg, 184 μmol, 1.0 eq.), Ru-Phos Pd G₁ (27 mg, 36.8 μmol, 0.2 eq.) and Ru-Phos (17 mg, 36.8 μmol, 0.2 eq.) in 1,4-Dioxane (2 mL) was stirred at 100° C. for 3 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-50% EtOAc in PE) to give methyl 5-(5-(1,3-dimethyl-2-oxo-7-(tetrahydro-2H-pyran-4-yl-3,4-d2)-1,2-dihydroquinolin-5-yl)-5,6,7,8-tetrahydropyrido[3,2-d]pyrimidin-2-yl)picolinate (50 mg, 52% yield) as a yellow solid.

LC-MS (ESI): mass calcd. for C₃₀H₂₉D₂N₅O₄, 527.25; m/z found, 528 [M+H]⁺.

### Step F: 5-(5-(1,3-dimethyl-2-oxo-7-(tetrahydro-2H-pyran-4-yl-3,4-d2)-1,2-dihydroquinolin-5-yl)-5,6,7,8-tetrahydropyrido[3,2-d]pyrimidin-2-yl)picolinic acid

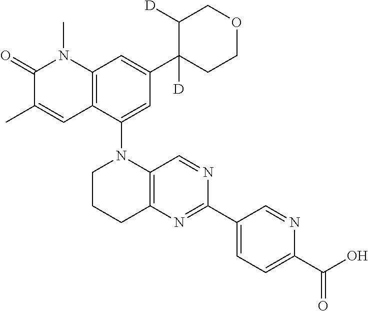 [SMILES: [2H]C1COCCC1([2H])c1cc(N2CCCc3nc(-c4ccc(C(=O)O)nc4)ncc32)c2cc(C)c(=O)n(C)c2c1]

A mixture of methyl 5-(5-(1,3-dimethyl-2-oxo-7-(tetrahydro-2H-pyran-4-yl-3,4-d2)-1,2-dihydroquinolin-5-yl)-5,6,7,8-tetrahydropyrido[3,2-d]pyrimidin-2-yl)picolinate (55 mg, 104 μmol, 1.0 eq.) and lithium hydroxide (3 mg, 104 μmol, 1.0 eq.) in THF (1 mL) and H₂O(1 mL) was stirred at 25° C. for 30 mins under N₂. The resulting solution was reduced pressure to give 5-(5-(1,3-dimethyl-2-oxo-7-(tetrahydro-2H-pyran-4-yl-3,4-d2)-1,2-dihydroquinolin-5-yl)-5,6,7,8-tetrahydropyrido[3,2-d]pyrimidin-2-yl)picolinic acid (30 mg, 56% yield) as a yellow solid.

LC-MS (ESI): mass calcd. for C₂₉H₂₇D₂N₅O₄, 513.23; m/z found, 514 [M+H]⁺.

### Intermediate 18. 3-(5-(3-aminoprop-1-yn-1-yl)benzo[b]thiophen-3-yl)piperidine-2,6-dione

### Step A: methyl 2-((4-bromophenyl)thio)acetate

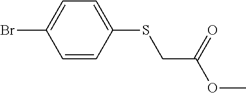

To a solution of 4-bromobenzenethiol (5.0 g, 26.4 mmol, 1 eq.) in THF (100 mL) was added methyl 2-bromoacetate (6.1 g, 39.7 mmol, 1.5 eq.) and TEA (7.4 mL, 52.9 mmol, 2 eq.) at 25° C. The mixture was then stirred at 70° C. for 4 hours under N₂ atmosphere. The resulting solution was diluted with 100 mL of water and extracted with EtOAc (100 mL×3). The combined organic layers were washed with brine, dried over Na₂SO₄, and concentrated under reduced pressure to give methyl 2-((4-bromophenyl)thio)acetate (6.9 g, crude). The product was used for next step directly without further purification.

¹H NMR (400 MHz, DMSO-d₆) δ 7.55-7.47 (m, 2H), 7.34-7.27 (m, 2H), 3.94 (s, 2H), 3.63 (s, 3H).

### Step B: 2-((4-bromophenyl)thio)acetic acid

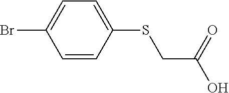 [SMILES: O=C(O)CSc1ccc(Br)cc1]

To a solution of methyl 2-((4-bromophenyl)thio)acetate (3.9 g, 14.9 mmol, 1 eq.) in MeOH:H₂O(99 mL, 10:1) was added NaH (1.2 g, 29.9 mmol, 2 eq. wt=60% in oil) at 25° C. The mixture was then stirred at 25° C. for 16 hours under N₂ atmosphere. The mixture was acidified with HCl (6 M in water) and extracted with EtOAc (100 mL×3). The combined organic layers were washed with brine, dried over Na₂SO₄, and concentrated under reduced pressure to give 2-((4-bromophenyl)thio)acetic acid (3.5 g, crude). The product was used for next step directly without further purification.

LC-MS (ESI): mass calcd. for C₈H₇BrO2S, 245.9; m/z found, 244.8/246.8 [M−H]-.

### Step C: 5-bromobenzo[b]thiophen-3(2H)-one

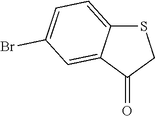 [SMILES: O=C1CSc2ccc(Br)cc21]

To a solution of 2-((4-bromophenyl)thio)acetic acid (3 g, 12.1 mmol, 1 eq.) in TfOH (15 mL) at 25° C. The mixture was then stirred at 80° C. for 30 mins under N₂ atmosphere. The resulting solution was diluted with 30 mL of water and extracted with EtOAc (30 mL×3). The combined organic layers were washed with brine, dried over Na₂SO₄, and concentrated under reduced pressure. The residue was purified by flash column chromatography on silica gel (100-200 mesh silica gel, 0-5% EA in PE) to give 5-bromobenzo[b]thiophen-3(2H)-one (1.2 g, 41% yield) as a yellow solid.

LC-MS (ESI): mass calcd. for C₈H₅BrOS, 227.9; m/z found, 229.0/231.0 [M+H]⁺.

¹H NMR (400 MHz, CDCl3) δ 7.89 (d, J=1.6 Hz, 1H), 7.64 (dd, J=8.4, 2.0 Hz, 1H), 7.31 (d, J=8.4 Hz, 1H), 3.83 (s, 2H).

### Step D: ethyl 2-(5-bromobenzo[b]thiophen-3-yl)acetate

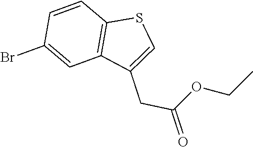 [SMILES: CCOC(=O)Cc1csc2ccc(Br)cc12]

To a solution of 5-bromobenzo[b]thiophen-3(2H)-one (400 mg, 1.75 mmol, 1 eq.) in Toluene (10 mL) was added ethyl 2-(triphenyl-{5}-phosphanylidene)acetate (608 mg, 1.75 mmol, 1 eq.) at 25° C. The mixture was then stirred at 110° C. for 16 hours under N₂ atmosphere. The resulting solution was diluted with 20 mL of water and extracted with EtOAc (20 mL×3). The combined organic layers were washed with brine, dried over Na₂SO₄, and concentrated under reduced pressure. The residue was purified by flash column chromatography on silica gel (100-200 mesh silica gel, 0-5% EtOAc in PE) to give ethyl 2-(5-bromobenzo[b]thiophen-3-yl)acetate (245 mg, 47% yield) as a yellow oil.

¹H NMR (400 MHz, CDCl3) δ 7.84 (d, J=1.8 Hz, 1H), 7.64 (d, J=8.5 Hz, 1H), 7.38 (dd, J=8.6, 1.8 Hz, 1H), 7.32 (s, 1H), 4.14-4.09 (m, 2H), 3.74 (d, J=0.7 Hz, 2H), 1.22-1.20 (m, 3H).

### Step E: 3-(5-bromobenzo[b]thiophen-3-yl)piperidine-2,6-dione

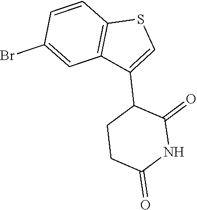 [SMILES: O=C1CCC(c2csc3ccc(Br)cc23)C(=O)N1]

To a solution of ethyl 2-(5-bromobenzo[b]thiophen-3-yl)acetate (200 mg, 668 μmol, 1 eq.) in DMF (5 mL) was added potassium 2-methylpropan-2-olate (75 mg, 668 μmol, 1 eq.) and acrylamide (190 mg, 2.67 mmol, 4 eq.) at 0° C. The mixture was then stirred at 25° C. for 2 hours under N₂ atmosphere. The resulting solution was diluted with 20 mL of water and extracted with EtOAc (20 mL×3). The combined organic layers were washed with brine, dried over Na₂SO₄, and concentrated under reduced pressure. The residue was purified by flash column chromatography on silica gel (100-200 mesh silica gel, 0-30% EtOAc in PE) to give 3-(5-bromobenzo[b]thiophen-3-yl)piperidine-2,6-dione (100 mg, 46% yield) as a yellow solid.

LC-MS (ESI): mass calcd. for C₁₃H₁₀BrNO2S, 323.0; m/z found, 323.9/325.9 [M+H]⁺.

### Step F: tert-butyl (3-(3-(2,6-dioxopiperidin-3-yl)benzo[b]thiophen-5-yl)prop-2-yn-1-yl)carbamate

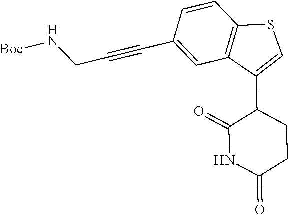 [SMILES: CC(C)(C)OC(=O)NCC#Cc1ccc2scc(C3CCC(=O)NC3=O)c2c1]

To a solution of 3-(5-bromobenzo[b]thiophen-3-yl)piperidine-2,6-dione (90 mg, 278 μmol, 1 eq.) in DMF (4 mL) was added Pd(PPh₃)₂C₁₂ (20 mg, 27.8 μmol, 0.1 eq.), CuI (5 mg, 27.8 μmol, 0.1 eq.), PCy³ (8 mg, 27.8 μmol, 0.1 eq.), TEA (387 μL, 2.78 mmol, 10 eq.) and tert-butyl prop-2-yn-1-ylcarbamate (129 mg, 833 μmol, 3 eq.) at 25° C. The mixture was then stirred at 80° C. for 2 hours under N₂ atmosphere. The resulting solution was diluted with 20 mL of water and extracted with EtOAc (20 mL×3). The combined organic layers were washed with brine, dried over Na₂SO₄, and concentrated under reduced pressure. The residue was purified by flash column chromatography on silica gel (100-200 mesh silica gel, 0-25% EtOAc in PE) to give tert-butyl (3-(3-(2,6-dioxopiperidin-3-yl)benzo[b]thiophen-5-yl)prop-2-yn-1-yl)carbamate (60 mg, 54% yield) as a yellow oil.

### LC-MS (ESI): mass calcd. for C₂₁H₂₂N₂O₄S, 398.1; m/z found, 397.0 [M−H]⁻

### Step G: 3-(5-(3-aminoprop-1-yn-1-yl)benzo[b]thiophen-3-yl)piperidine-2,6-dione

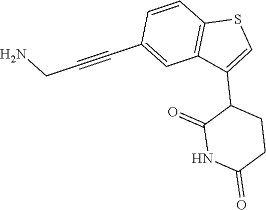 [SMILES: NCC#Cc1ccc2scc(C3CCC(=O)NC3=O)c2c1]

To a solution of tert-butyl (3-(3-(2,6-dioxopiperidin-3-yl)benzo[b]thiophen-5-yl)prop-2-yn-1-yl)carbamate (63 mg, 158 μmol, 1 eq.) in DCM (4 mL) was added TFA (0.4 mL) at 0° C. The mixture was then stirred at 25° C. for 1 hour under N₂ atmosphere. The resulting solution was concentrated under vacuum to give 3-(5-(3-aminoprop-1-yn-1-yl)benzo[b]thiophen-3-yl)piperidine-2,6-dione (47 mg, crude) and which was used for next step directly without further purification. LC-MS (ESI): mass calcd. for C₁₆H₁₄N₂O₂S, 298.1; m/z found, 299.2 [M+H]⁺.

### Intermediate 19. 5-(5-(7-(diethylamino)-1,3-dimethyl-2-oxo-1,2-dihydro-1,6-naphthyridin-5-yl)-5,6,7,8-tetrahydropyrido[3,2-d]pyrimidin-2-yl)picolinic acid

### Step A: methyl 5-(5-(7-chloro-1,3-dimethyl-2-oxo-1,2-dihydro-1,6-naphthyridin-5-yl)-5,6,7,8-tetrahydropyrido[3,2-d]pyrimidin-2-yl)picolinate

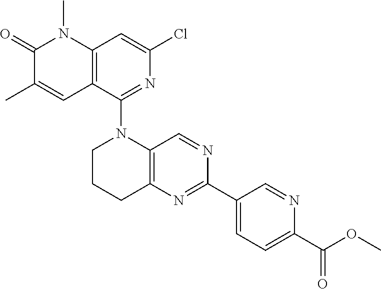

To a solution of 5,7-dichloro-1,3-dimethyl-1,6-naphthyridin-2(1H)-one (100 mg, 411 μmol, 1.2 eq.), Pd₂(dba)₃ (31 mg, 34.28 μmol, 0.1 eq.), methyl 5-(5,6,7,8-tetrahydropyrido[3,2-d]pyrimidin-2-yl)picolinate (93 mg, 343 μmol, 1.0 eq.) and Xant-Phos (40 mg, 69 μmol, 0.2 eq.) in 1,4-Dioxane (5 mL) was added K₂CO₃ (142 mg, 1 mmol, 3,0 eq.) at 25° C.

The mixture was stirred at 100° C. for 2 hours under N₂ atmosphere. The reaction was monitored by LCMS. The resulting solution was diluted with 50 ml of water and extracted with EtOAc (3×50 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford methyl 5-(5-(7-chloro-1,3-dimethyl-2-oxo-1,2-dihydro-1,6-naphthyridin-5-yl)-5,6,7,8-tetrahydropyrido[3,2-d]pyrimidin-2-yl)picolinate (60 mg, yield 37%) as a yellow solid.

LC-MS (ESI): mass calcd. for C₂₄H₂₁ClN₆O₃, 476.1; m/z found, 477.1 [M+H]⁺.

### Step B: methyl 5-(5-(7-(diethylamino)-1,3-dimethyl-2-oxo-1,2-dihydro-1,6-naphthyridin-5-yl)-5,6,7,8-tetrahydropyrido[3,2-d]pyrimidin-2-yl)picolinate

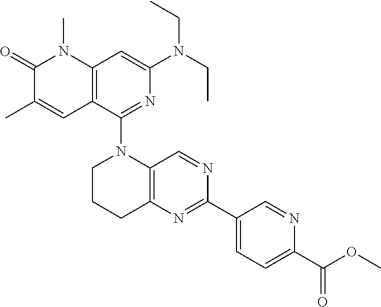

To a solution of methyl 5-(5-(7-chloro-1,3-dimethyl-2-oxo-1,2-dihydro-1,6-naphthyridin-5-yl)-5,6,7,8-tetrahydropyrido[3,2-d]pyrimidin-2-yl)picolinate (60 mg, 126 μmol, 1.0 eq.), Pd₂(dba)₃ (12 mg, 13 μmol, 0.1 eq.), diethylamine (138 mg, 1.9 mmol, 15.0 eq.) and Xant-Phos (14.6 mg, 25 μmol, 0.2 eq.) in dioxane (3 mL) was added Cs₂CO₃ (123 mg, 377 μmol, 3.0 eq.) at 25° C. The mixture was stirred at 100° C. for 10 hours under N₂ atmosphere. The reaction was monitored by LCMS. The resulting solution was diluted with 20 ml of water, then extracted with EtOAc (3×20 mL) and washed with brine. The combined organic layer was dried over anhydrous sodium sulfate and concentrated under reduced pressure. The residue was purified by flash column chromatography on silica gel (100-200 mesh silica gel, 0-100% EtOAc in PE) to afford productmethyl 5-(5-(7-(diethylamino)-1,3-dimethyl-2-oxo-1,2-dihydro-1,6-naphthyridin-5-yl)-5,6,7,8-tetrahydropyrido[3,2-d]pyrimidin-2-yl)picolinate (22 mg, yield 35%) as a yellow solid. LC-MS (ESI): mass calcd. for C₂₈H₃₁N₇O₃, 513.3; m/z found, 514.3 [M+H]⁺.

### Step C: 5-(5-(7-(diethylamino)-1,3-dimethyl-2-oxo-1,2-dihydro-1,6-naphthyridin-5-yl)-5,6,7,8-tetrahydropyrido[3,2-d]pyrimidin-2-yl)picolinic acid

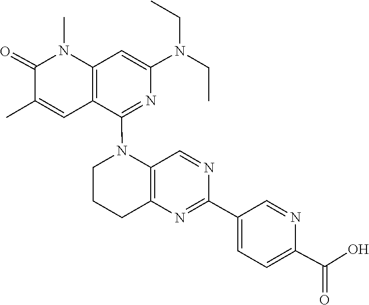 [SMILES: CCN(CC)c1cc2c(cc(C)c(=O)n2C)c(N2CCCc3nc(-c4ccc(C(=O)O)nc4)ncc32)n1]

To a solution of methyl 5-(5-(7-(diethylamino)-1,3-dimethyl-2-oxo-1,2-dihydro-1,6-naphthyridin-5-yl)-5,6,7,8-tetrahydropyrido[3,2-d]pyrimidin-2-yl)picolinate (22 mg, 43 μmol, 1.0 eq.) in THF (2 mL) and H₂O(2 mL) was added LiGH (2 mg, 86 μmol, 2.0 eq.) at 25° C. The mixture was then stirred at 25° C. for 1 hour. LCMS showed the reaction was completed. The reaction was concentrated under vacuum and which was used in the next step without further purification. LC-MS (ESI): mass calcd. for C₂₈H₃MN₇O₃, 512.3; m/z found, 513.3 [M+H]⁺.

### Intermediate 20. 5-bromo-1,3,7-trimethylquinolin-2(1H)-one

### Step A: (E)-3-bromo-N,5-dimethyl-2-((methylimino)methyl)aniline

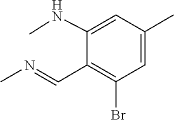

To a solution of 2-bromo-6-fluoro-4-methylbenzaldehyde (5.0 g, 23.0 mmol, 1.0 eq.) in MeNH₂ (20 mL, 69.1 mmol, 3 eq., 3M in EtOH.) was stirred at 80° C. for 16 hours. The mixture was diluted water and extracted with EtOAC (50 mL×3). The combined organic layers were washed with brine (50 mL), dried over Na₂SO₄, filtered and concentrated. The residue was purified by flash chromatography (100-200 mesh silica gel, 0% ˜10% EtOAC in PE) to afford (E)-3-bromo-N,5-dimethyl-2-((methylimino)methyl)aniline (2.3 g, yield 41%) as a yellow solid.

LC-MS (ESI): mass calcd. for C₁₀H₁₃BrN₂, 240.0/242.0; m/z found, 241.0/243.1 [M+H]⁺.

### Step B: 2-bromo-4-methyl-6-(methylamino)benzaldehyde

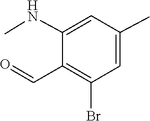

To a solution of (E)-3-bromo-N,5-dimethyl-2-((methylimino)methyl)aniline (2.3 g, 9.54 mmol, 1 eq.) in 2N HCl (35 mL) was stirred at 25° C. for 2 hours under N₂. The resulting solution was quenched with 100 mL of aq. NaHCO₃ and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-10% EtOAc in PE) to give 2-bromo-4-methyl-6-(methylamino)benzaldehyde (1.8 g, yield 83%) as a yellow solid.

LC-MS (ESI): mass calcd. for C₉H₁₀BrNO, 226.9/228.9; m/z found, 227.9/229.9 [M+H]⁺.

### Step C: N-(3-bromo-2-formyl-5-methylphenyl)-N-methylpropionamide

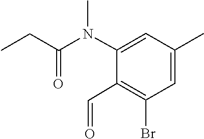 [SMILES: CCC(=O)N(C)c1cc(C)cc(Br)c1C=O]

A mixture of 2-bromo-4-methyl-6-(methylamino)benzaldehyde (1.8 g, 7.89 mmol, 1.0 eq.), Cs₂CO₃ (5.1 g, 15.8 mmol, 2.0 eq.) and propionyl chloride (1.1 g, 11.8 mmol, 1.5 eq.) in DMF (15 mL) was stirred at 60° C. for 1.5 hours under N₂. The resulting solution was quenched with 50 mL of sat. NH₄Cl and extracted with EtOAc (50 mL×3). The combined organic layers were washed with brine (50 mL), dried over Na₂SO₄ and concentrated under reduced pressure.

The residue was purified by flash column chromatography on silica gel (100-200 mesh silica gel, 0-50% EtOAc in PE) to give N-(3-bromo-2-formyl-5-methylphenyl)-N-methylpropionamide (800 mg, yield 36%) as a yellow oil. LC-MS (ESI): mass calcd. for C₁₂H₁₄BrNO₂, 283.0/285.0; m/z found, 284.2/286.2 [M+H]⁺.

### Step D: 5-bromo-1,3,7-trimethylquinolin-2(1H)-one

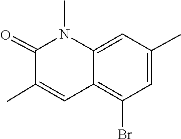

A mixture of N-(3-bromo-2-formyl-5-methylphenyl)-N-methylpropionamide (700 mg, 2.46 mmol, 1.0 eq.), Cs₂CO₃ (1.6 g, 4.93 mmol, 2.0 eq.) in MeOH (10 mL) was stirred at 80° C. for 2 hours under N₂. The resulting solution was quenched with 50 mL of sat. NH₄Cl and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-50% EtOAc in PE) to give 5-bromo-1,3,7-trimethylquinolin-2(1H)-one (280 mg, yield 43%) as a yellow solid.

LC-MS (ESI): mass calcd. for C₁₂H₁₂BrNO, 265.1/267.1; m/z found, 266.0/268.0 [M+H]⁺.

### Intermediate 21. Methyl 3-methyl-5-(8-methyl-5,6,7,8-tetrahydropteridin-2-yl)picolinate

### Step A: N-(2-chloro-5-nitropyrimidin-4-yl)-N-methylglycinate

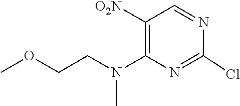 [SMILES: COCCN(C)c1nc(Cl)ncc1[N+](=O)[O-]]

To a solution of 2,4-dichloro-5-nitropyrimidine (5 g, 26 mmol, 1.0 eq.) in DCM (100 mL) was added TEA (7 mL, 51.6 mmol, 2.0 eq.) and methyl methylglycinate (2.6 g, 26 mmol, 1.0 eq.) at 0° C. The mixture was stirred at 0° C. for 15 mins under nitrogen atmosphere. The reaction was monitored by LCMS. The mixture was diluted water and extracted with EtOAC (300 mL×3). The combined organic layers were washed with brine (300 mL×2), dried over Na₂SO₄, filtered and concentrated. The residue was purified by flash chromatography (100-200 mesh silica gel, 0%-10% EtOAC in PE) to afford the product methyl N-(2-chloro-5-nitropyrimidin-4-yl)-N-methylglycinate (4 g, yield 59%) as a yellow solid. LC-MS (ESI): mass calcd. for C₈H₁₁ClN₄O₃, 246.0; m/z found, 247.0 [M+H]⁺.

### Step B: 2-chloro-8-methyl-7,8-dihydropteridin-6(5H)-one

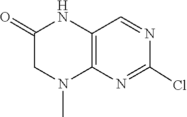

To a solution of methyl N-(2-chloro-5-nitropyrimidin-4-yl)-N-methylglycinate (4 g, 15 mmol 1.0 eq.), Iron (4.3 g, 77 mmol, 5.0 eq.) in AcOH (40 mL). The reaction mixture was stirred at 80° C. for 2 hours. The mixture was diluted water and extracted with EtOAC (200 mL×3). The combined organic layers were washed with brine (100 mL), dried over Na₂SO₄, filtered and concentrated. The residue was purified by flash chromatography (100-200 mesh silica gel, 0-30% EtOAc in PE) to give 2-chloro-8-methyl-7,8-dihydropteridin-6(5H)-one (900 mg, yield 29%) as a white solid. LC-MS (ESI): mass calcd. for C₇H₇ClN₄O, 198.0; m/z found, 199.0 [M+H]⁺.

### Step C: 2-chloro-8-methyl-5,6,7,8-tetrahydropteridine

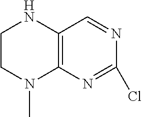

To a solution of 2-chloro-8-methyl-7,8-dihydropteridin-6(5H)-one (695 mg, 3.5 mmol, 1.0 eq.), Boranetetrahydrofurancomplexsolution (12 mL, 12 mmol, 3.4 eq., 1 M in Tol) in THF (12 mL). The reaction mixture was stirred at 60° C. for 1 hour. The mixture was diluted water and extracted with EtOAC (60 mL×3). The combined organic layers were washed with brine (50 mL), dried over Na₂SO₄, filtered and concentrated. The residue was purified by flash chromatography (100-200 mesh silica gel, 0-50% EtOAc in PE) 2-chloro-8-methyl-5,6,7,8-tetrahydropteridine (0.4 g, yield 54%) as a yellow solid. LC-MS (ESI): mass calcd. for C₇H₉ClN₄, 180.0; m/z found, 181.0 [M+H]⁺.

### Step D: methyl 3-methyl-5-(8-methyl-5,6,7,8-tetrahydropteridin-2-yl)picolinate

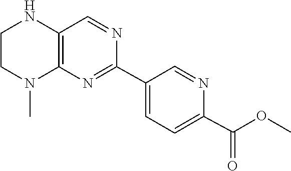 [SMILES: COC(=O)c1ccc(-c2ncc3c(n2)N(C)CCN3)cn1]

To a mixture of 2-chloro-8-methyl-5,6,7,8-tetrahydropteridine (120 mg, 650 μmol, 1.0 eq.), methyl 3-methyl-5-(4,4,5,5-tetramethyl-1,3,2-dioxaborolan-2-yl)picolinate (270 mg, 975 mol, 1.5 eq.), 1,1′-Bis(di-t-butylphosphino)ferrocene palladium dichloride (84.7 mg, 130 μmol, 0.2 eq.) in 1,4-Dioxane (5 mL) and Water (0.5 mL) was added K₃PO₄ (414 mg, 1.9 mmol, 3.0 eq.).

The reaction mixture was purged with nitrogen three times, then heated to 100° C. for 2 hours under nitrogen. The mixture was diluted water and extracted with EtOAC (60 mL×3). The combined organic layers were washed with brine (100 mL), dried over Na₂SO₄, filtered and concentrated. The residue was purified by flash chromatography (100-200 mesh silica gel, 0-60% EtOAc in PE) to give methyl 3-methyl-5-(8-methyl-5,6,7,8-tetrahydropteridin-2-yl)picolinate (90 mg, yleld 46%) as a yellow solid. LC-MS (ESI): mass calcd. for C₁₄H₁₅N₅O₂, 285.1; m/z found, 286.1 [M+H]⁺.

### Intermediate 22. 3-Chloro-5,6,7,8-tetrahydropyrido[2,3-c]pyridazine

### Step A: tert-butyl (3-(3,6-dichloropyridazin-4-yl)propyl)carbamate

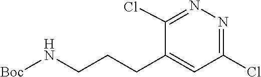 [SMILES: CC(C)(C)OC(=O)NCCCc1cc(Cl)nnc1Cl]

To a solution of 3,6-dichloropyridazine (2 g, 13.4 mmol, 1.0 eq.) in DMSO (26 mL) was added 4CzIPN (212 mg, 269 μmol, 0.02 eq.), Benzoyl peroxide (5.2 mL, 27 mmol, 2.0 eq.), 4-((tert-butoxycarbonyl)amino)butanoic acid (3.3 g, 16.1 mmol, 1.2 eq.) and molecular sieves pack 4A (1.6 g,13.4 mmol, 1.0 eq.) under visible light irradiation (White LED, 6500 K), the solution was stirred at 25° C. for 16 hours in a PhotoRedOx Box (EvoluChem™). The resulting solution was diluted with 100 mL of water and extracted with EtOAc (3×100 mL). The combined organic layers were washed with brine (3×100 mL), dried over Na₂SO₄, and concentrated under reduced pressure. The residue was purified by flash chromatography on silica gel to afford tert-butyl (3-(3,6-dichloropyridazin-4-yl)propyl)carbamate (2.2 g, yield 51%) as colorless oil. LC-MS (ESI): mass calcd. for C₁₂H₁₇Cl₂N₃O₂, 305.2; m/z found, 250.2 [M+H−56]+.

### Step B: 3-(3,6-dichloropyridazin-4-yl)propan-1-amine

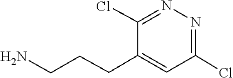 [SMILES: NCCCc1cc(Cl)nnc1Cl]

A solution of tert-butyl (3-(3,6-dichloropyridazin-4-yl)propyl)carbamate (2.2 g, 7.8 mmol, 1.0 eq.) in HCl in dioxane (20 mL) was stirred at 25° C. for 1 hour to give white solution.

LCMS showed the reaction was completed. The solution was concentrated to afford 3-(3,6-dichloropyridazin-4-yl)propan-1-amine (1.5 g, yield 94%) as a white solid. LC-MS (ESI): mass calcd. for C₇H₉C₁₂N₃, 205.1; m/z found, 206.1 [M+H]⁺.

### Step C: 3-chloro-5,6,7,8-tetrahydropyrido[2,3-c]pyridazine

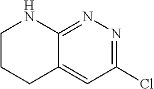 [SMILES: Clc1cc2c(nn1)NCCC2]

To a solution of 3-(3,6-dichloropyridazin-4-yl)propan-1-amine (600 mg, 2.9 mmol, 1.0 eq.) in BuOH (10 mL) was added DIEA (2.5 mL, 14.6 mmol, 5.0 eq.). The solution was stirred at 100° C. for 16 hours to give yellow solution. LCMS showed the reaction was completed. The resulting solution was diluted with 100 mL of water and extracted with EtOAc (100 mL). The combined organic layers were washed with brine (100 mL), dried over Na₂SO₄, and concentrated under reduced pressure. The residue was purified by flash chromatography (100-200 mesh silica gel, 0% ˜100% EtOAc in PE) to afford 3-chloro-5,6,7,8-tetrahydropyrido[2,3-c]pyridazine (120 mg, yield 24%) as a yellow solid. LC-MS (ESI): mass calcd. for C₇H₈ClN₃, 169.6; m/z found, 170.6 [M+H]⁺.

### Intermediate 23. Methyl 5-(8-(7-hydroxy-1,3-dimethyl-2-oxo-1,2-dihydroquinolin-5-yl)isoquinolin-3-yl)picolinic acid

### Step A: N-(2-bromobenzyl)-2-hydroxypropanamide

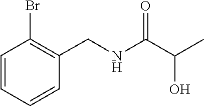

A mixture of (2-bromophenyl)methanamine (25 g, 134 mmol, 1.2 eq.) and methyl 2-hydroxypropanoate (12 g, 112 mmol, 1.0 eq.) in reaction vessel. The mixture was stirred at 130° C. for 6 hours under N₂. LCMS showed the reaction was completed. The resulting solution concentrated under reduced pressure to give N-(2-bromobenzyl)-2-hydroxypropanamide (35 g, yield 73%) as a yellow solid. LC-MS (ESI): mass calcd. for C₁₀H₁₂BrNO₂, 257.0/259.0; m/z found, 258/260.0 [M+H]⁺.

### Step B: N-(2-bromobenzyl)-2-oxopropanamide

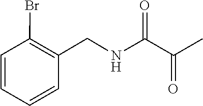

A solution of N-(2-bromobenzyl)-2-hydroxypropanamide (35.0 g, 136 mmol, 1.0 eq.) and DMP (58 g, 136 mmol, 1.0 eq.) in DCM (400 mL) was stirred at 25° C. for 30 mins under N₂ atmosphere. After cooled to room temperature, the resulting solution was diluted with 300 mL of water and extracted with EA (500 mL×3). The combined organic layers were washed with brine (200 mL×2), dried over Na₂SO₄ and concentrated under reduced pressure. The residue was purified by flash column chromatography on silica gel (100-200 mesh silica gel, 0-20% EtOAc in PE) to give N-(2-bromobenzyl)-2-oxopropanamide (17 g, yield 49%) as a yellow solid. LC-MS (ESI): mass calcd. for C₁₀H₁₀BrNO₂, 255.0/257.0; m/z found, 256.0/257.0. [M+H]⁺.

### Step C: 8-bromo-4-methylisoquinolin-3-ol

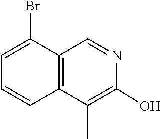

H₂SO₄ (25 mL) was dropped in N-(2-bromobenzyl)-2-oxopropanamide (9 g, 35 mmol, 1.0 eq.) at 0° C. The mixture was stirred at 60° C. for 16 hours under N₂ atmosphere. LCMS showed desired MS. The reaction mixture was then slowly poured over ice. The resulting slurry in a 500 mL Erlenmeyer flask was cooled in an ice bath and rendered basic (pH 10) with the dropwise addition of NaHCO₃. The resulting solution was diluted with 300 mL of water and extracted with EA (200 mL×3). The combined organic layers were washed with brine (150 mL×2), dried over Na₂SO₄ and concentrated under reduced pressure. The residue was purified by flash column chromatography on silica gel (100-200 mesh silica gel, 0-30% EtOAc in PE) to give 8-bromo-4-methylisoquinolin-3-ol (300 mg, yield 3.6%) as a yellow solid.

LC-MS (ESI): mass calcd. for C₁₀HsBrNO, 237/239; m/z found, 238/240 [M+H]⁺.

### Step D: 5-(3-hydroxy-4-methylisoquinolin-8-yl)-1,3-dimethyl-1,3-dihydro-2H-benzo[d]imidazol-2-one

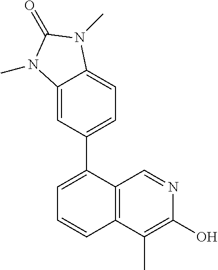 [SMILES: Cc1c(O)ncc2c(-c3ccc4c(c3)n(C)c(=O)n4C)cccc12]

A mixture of 8-bromo-4-methylisoquinolin-3-ol (120 mg, 504 μmol, 1.0 eq.), PdC12(dppf) (148 mg, 202 μmol, 0.4 eq.), 1,3-dimethyl-5-(4,4,5,5-tetramethyl-1,3,2-dioxaborolan-2-yl)-1,3-dihydro-2H-benzo[d]imidazol-2-one (174 mg, 605 μmol, 1.2 eq.) and K₂CO₃ (209 mg, 1.5 mmol, 3.0 eq.) in 1,4-Dioxane (10 mL) and Water (0.5 mL). The mixture was stirred at 100° C. for 6 hours under N₂. The resulting solution was diluted with 50 mL of H₂O and extracted with EtOAc (60 mL×3). The combined organic layers were washed with brine, dried over Na₂SO₄, and concentrated under reduced pressure. The residue was purified by flash column chromatography on silica gel (100-200 mesh silica gel, 0-70% EtOAc in PE) to give 5-(3-hydroxy-4-methylisoquinolin-8-yl)-1,3-dimethyl-1,3-dihydro-2H-benzo[d]imidazol-2-one (40.0 mg, yield 24.8%) as a yellow solid.

LC-MS (ESI): mass calcd. for C₁₉H₁₇N₃O₂, 319.1; m/z found, 320.1 [M+H]⁺.

### Step E: methyl 5-(8-(1,3-dimethyl-2-oxo-7-(((trifluoromethyl)sulfonyl)oxy)-1,2-dihydroquinolin-5-yl)isoquinolin-3-yl)picolinate

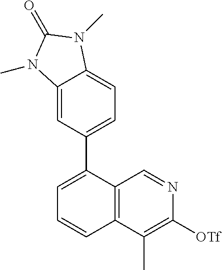 [SMILES: Cc1c(OS(=O)(=O)C(F)(F)F)ncc2c(-c3ccc4c(c3)n(C)c(=O)n4C)cccc12]

A mixture of 5-(3-hydroxy-4-methylisoquinolin-8-yl)-1,3-dimethyl-1,3-dihydro-2H-benzo[d]imidazol-2-one (40 mg, 125 μmol, 1.0 eq.) and TEA (69.8, 501 μmol, 4.0 eq.) in DCM (4 mL) was added Tf₂O (0.03 mL, 251 μmol, 2.0 eq.). The mixture was stirred at 25° C. for 60 mins under N₂. The resulting solution was diluted with 20 mL of H₂O and extracted with EtOAc (20 mL×3). The combined organic layers were washed with brine, dried over Na₂SO₄, and concentrated under reduced pressure. The residue was purified by flash column chromatography on silica gel (100-200 mesh silica gel, 0-70% EtOAc in PE) to give 8-(1,3-dimethyl-2-oxo-2,3-dihydro-1H-benzo[d]imidazol-5-yl)-4-methylisoquinolin-3-yl trifluoromethanesulfonate (40 mg, yield 35.4%) as a yellow solid.

LC-MS (ESI): mass calcd. for C₂₈H₂₀F3N₃₀₆S, 451.1; m/z found, 452.1 [M+H]⁺.

### Step F: methyl 5-(8-(7-(4-hydroxy-4-methylpiperidin-1-yl)-1,3-dimethyl-2-oxo-1,2-dihydroquinolin-5-yl)isoquinolin-3-yl)picolinate

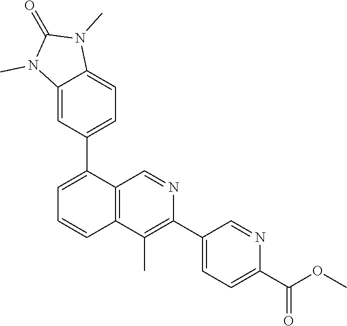

A mixture of 8-(1,3-dimethyl-2-oxo-2,3-dihydro-1H-benzo[d]imidazol-5-yl)-4-methylisoquinolin-3-yl trifluoromethanesulfonate (40 mg, 88 μmol, 1.0 eq.), PdC12(dppf) (13 mg, 17 μmol, 0.2 eq.), methyl 5-(4,4,5,5-tetramethyl-1,3,2-dioxaborolan-2-yl)picolinate (117 mg, 443 μmol, 5.0 eq.) and K₂CO₃ (37 mg, 266 μmol, 3.0 eq.) in Water (0.2 mL) and 1,4-Dioxane (4 mL).

The mixture was stirred at 100° C. for 30 min under N₂. The resulting solution was diluted with 50 mL of H₂O and extracted with EtOAc (60 mL×3). The combined organic layers were washed with brine, dried over Na₂SO₄, and concentrated under reduced pressure. The residue was purified by flash column chromatography on silica gel (100-200 mesh silica gel, 0-70% EtOAc in PE) to give methyl 5-(8-(1,3-dimethyl-2-oxo-2,3-dihydro-1H-benzo[d]imidazol-5-yl)-4-methylisoquinolin-3-yl)picolinate (15 mg, yield 38%) as a yellow solid. LC-MS (ESI): mass calcd. for C₂₆H₂₂N₄O₃, 438.1; m/z found, 439.1 [M+H]⁺.

### Step G: methyl 5-(8-(7-hydroxy-1,3-dimethyl-2-oxo-1,2-dihydroquinolin-5-yl)isoquinolin-3-yl)picolinic acid

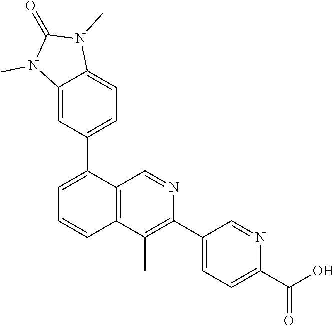 [SMILES: Cc1c(-c2ccc(C(=O)O)nc2)ncc2c(-c3ccc4c(c3)n(C)c(=O)n4C)cccc12]

To a solution of methyl 5-(8-(1,3-dimethyl-2-oxo-2,3-dihydro-1H-benzo[d]imidazol-5-yl)-4-methylisoquinolin-3-yl)picolinate (15 mg, 33 μmol, 1.0 eq.) in THF (1 mL) and Water (1 mL) was added lithium hydroxide (1.5 mg, 45.6 μmol, 2.0 eq.) at 25° C. The mixture was then stirred at 25° C. for 0.5 hour under N₂. The reaction mixture was concentrated under vacuum to afford 5-(8-(1,3-dimethyl-2-oxo-2,3-dihydro-1H-benzo[d]imidazol-5-yl)-4-methylisoquinolin-3-yl)picolinic acid (15 mg, crude) as a LiGH salt.

LC-MS (ESI): mass calcd. for C₂₅H₂₀N₄O₃, 424.1; m/z found, 425.1 [M+H]⁺.

### Intermediate 24. methyl 5′-methyl-4-(methylamino)-2-oxo-2H-[1,3′-bipyridine]-6′-carboxylate

### Step A: methyl 4-bromo-5′-methyl-2-oxo-2H-[1,3′-bipyridine]-6′-carboxylate

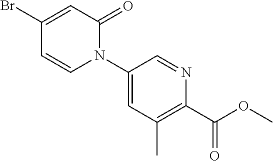 [SMILES: COC(=O)c1ncc(-n2ccc(Br)cc2=O)cc1C]

To a solution of 4-bromopyridin-2(1H)-one (1.2 g, 6.9 mmol, 1.0 eq.) and methyl 3-methyl-5-(4,4,5,5-tetramethyl-1,3,2-dioxaborolan-2-yl)picolinate (2.3 g, 8.3 mmol, 1.2 eq.) in Pyridine (60 mL) were added Cu(OAc)₂ (3.8 g, 20.7 mmol, 3.0 eq.) and Molecular sieves, 4A. The reaction mixture was stirred at 68° C. for 7 hours. The mixture was quenched water (40 mL), extracted with EtOAc (40 mL×3) and the layers were separated. The organic layers were washed with brine (50 mL), dried over Na₂SO₄, filtered and concentrated. The residue was purified by flash chromatography (100-200 mesh silica gel, 0% ˜3% DCM in MeOH) to afford methyl 4-bromo-5′-methyl-2-oxo-2H-[1,3′-bipyridine]-6′-carboxylate (300 mg, yield 13%) as a white solid. LC-MS (ESI): mass calcd. for C₁₃H₁₁BrN₂O₃, 321.9/323.9; m/z found, 323.1/325.1 [M+H]⁺.

### Step B: methyl 4-((tert-butoxycarbonyl)(methyl)amino)-5′-methyl-2-oxo-2H-[1,3′-bipyridine]-6′-carboxylate

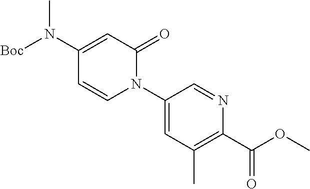 [SMILES: COC(=O)c1ncc(-n2ccc(N(C)C(=O)OC(C)(C)C)cc2=O)cc1C]

A mixture of methyl 4-bromo-5′-methyl-2-oxo-2H-[1,3′-bipyridine]-6′-carboxylate (300 mg, 928 μmol, 1.0 eq.), tert-butyl methylcarbamate (134 mg, 1 mmol, 1.1 eq.), PdOAc₂ (21 mg, 93 μmol, 0.1 eq.), Xantphos (54 mg, 93 μmol, 0.1 eq.) and Cs₂CO₃ (303 mg, 928 μmol, 1.0 eq.) in 1,4-Dioxane (5 mL) was stirred at 100° C. for 6 hours under N₂. The mixture was diluted water and extracted with EtOAc (20 mL×3). The combined organic layers were washed with brine (20 mL), dried over Na₂SO₄, filtered and concentrated. The residue was purified by flash column chromatography on silica gel (100-200 mesh silica gel, 0-60% EtOAc in PE) to give methyl 4-((tert-butoxycarbonyl)(methyl)amino)-5′-methyl-2-oxo-2H-[1,3′-bipyridine]-6′-carboxylate (120 mg, yield 35%) as a yellow solid. LC-MS (ESI): mass calcd. for C₁₉H₂₃N₃O₅,373.2; m/z found, 374 [M+H]⁺.

### Step C: methyl 5′-methyl-4-(methylamino)-2-oxo-2H-[1,3′-bipyridine]-6′-carboxylate

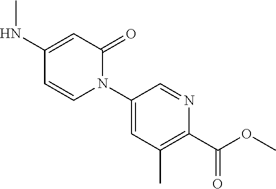 [SMILES: CNc1ccn(-c2cnc(C(=O)OC)c(C)c2)c(=O)c1]

A mixture of methyl 4-((tert-butoxycarbonyl)(methyl)amino)-5′-methyl-2-oxo-2H-[1,3′-bipyridine]-6′-carboxylate (120 mg, 321 μmol, 1.0 eq.) in DCM (5 mL) and TFA (1 mL) was stirred at 25° C. for 1 hour. The mixture was concentrated to afford methyl 5′-methyl-4-(methylamino)-2-oxo-2H-[1,3′-bipyridine]-6′-carboxylate (80 mg, yield 91%) as a yellow solid.

LC-MS (ESI): mass calcd. for C₁₄H₁₅N₃O₃,273.1; m/z found, 274 [M+H]⁺.

### Intermediate 25. 3-(5-iodobenzofuran-3-yl)piperidine-2,6-dione

### Step A: ethyl 2-(5-iodobenzofuran-3-yl)acetate

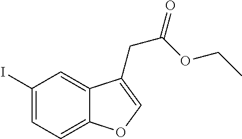 [SMILES: CCOC(=O)Cc1coc2ccc(I)cc12]

A mixture of ethyl 2-(5-bromobenzofuran-3-yl)acetate (5 g, 17.7 mmol, 1.0 eq.), (1S,2S)—N¹,N²-dimethylcyclohexane-1,2-diamine (202 mg, 1.8 mmol, 0.1 eq.), CuI (168 mg, 883 μmol, 0.05 eq.) and NaI (5.3 g, 35.3 mmol, 2.0 eq.) in 1,4-Dioxane (50 mL) was stirred at 110° C. for 20 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-10% EtOAc in PE) to give ethyl 2-(5-iodobenzofuran-3-yl)acetate (4 g, 69% yield) as a yellow solid. LC-MS (ESI): mass calcd. for C₁₂H₁₁IO₃, 329.98; m/z found, 330 [M+H]⁺.

### Step B: 3-(5-iodobenzofuran-3-yl)piperidine-2,6-dione

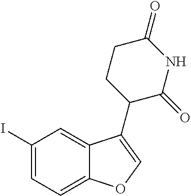 [SMILES: O=C1CCC(c2coc3ccc(I)cc23)C(=O)N1]

To a solution of ethyl 2-(5-iodobenzofuran-3-yl)acetate (4.0 g, 12.1 mmol, 1.0 eq.), t-BuOK (1.6 g, 14.5 mmol, 1.2 eq.) in DMF (40 mL) was added acrylamide (1.0 g, 14.5 mmol, 1.2 eq.) at 0° C. The mixture was stirred at 25° C. for 2 hours under N₂. The resulting solution was quenched sat. NH₄Cl and filtered to give 3-(5-iodobenzofuran-3-yl)piperidine-2,6-dione (4 g, 93% yield) as a yellow solid. LC-MS (ESI): mass calcd. for C₁₃H₁₀INO₃, 354.97; m/z found, 356 [M+H]⁺.

### Intermediate 26. 3-(7-fluoro-2-iodobenzofuran-4-yl)piperidine-2,6-dione

### Step A: 7-fluoro-2-iodobenzofuran-4-carboxylate

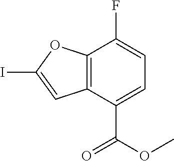

To a mixture of methyl 7-fluorobenzofuran-4-carboxylate (3.3 g, 1.7 mmol, 1.0 eq.) in THF (50 mL) was added LDA (9 mL, 1.8 mmol, 1.2 eq. 2 μmol/L in THF) at −78° C., the mixture was stirred at −78° C. for 30 mins, then a solution was 12 (570 mg, 2.2 mmol, 1.3 eq.) in THE was added this mixture and stirred at 25° C. for 2 hours. The resulting solution was diluted with 50 mL of H₂O and extracted with EtOAc (60 mL×3). The combined organic layers were washed with brine, dried over Na₂SO₄, and concentrated under reduced pressure. The residue was purified by flash column chromatography on silica gel (100-200 mesh silica gel, 0-10% EtOAc in PE) to give methyl 7-fluoro-2-iodobenzofuran-4-carboxylate (1.8 g, yield 33.1%) as a yellow oil. LC-MS (ESI): mass calcd. for C₁₀H₆FIO₃, 319.9; m/z found, 320.9 [M+H]⁺.

### Step B: (7-fluoro-2-iodobenzofuran-4-yl)methanol

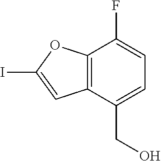 [SMILES: OCc1ccc(F)c2oc(I)cc12]

To a mixture of methyl 7-fluoro-2-iodobenzofuran-4-carboxylate (1.6 g, 5 mmol, 1.0 eq.) in THF (20 mL) was added DIBAL-H (4 mL, 6 μmol, 1.2 eq., 1.5 μmol/L in THF) at −78° C., the mixture was stirred at −78° C. for 30 mins. The mixture was quenched Potassium sodium tartrate tetrahydrate solution, extracted with EtOAC (100 mL×3) and the layers were separated. The organic layer was washed with brine (100 mL×3), dried over Na₂SO₄, filtered and concentrated. The product was purified by flash chromatography (100-200 mesh silica gel, 0% ˜20%EtOAC in PE) to afford (7-fluoro-2-iodobenzofuran-4-yl)methanol (1 g, yield 70%) as a yellow solid. ¹H NMR (400 MHz, DMSO) δ 7.40 (d, J=3.0 Hz, 1H), 7.18 (dd, J=8.3, 4.5 Hz, 1H), 7.11 (d, J=10.9, 8.3 Hz, 1H), 5.31 (t, J=5.7 Hz, 1H), 4.66 (d, J=5.7 Hz, 2H).

### Step C: 2-(7-fluoro-2-iodobenzofuran-4-yl)acetonitrile

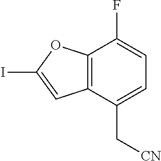 [SMILES: N#CCc1ccc(F)c2oc(I)cc12]

To a solution of (7-fluoro-2-iodobenzofuran-4-yl)methanol (1 g, 3.4 mmol, 1.0 eq.), Tributyl phosphine (1.7 mL, 6.8 mmol, 2.0 eq) and 2-hydroxy-2-methylpropanenitrile (437 mg, 5 mmol, 2.0 eq) in THF (20 mL) at 0° C. for 0.5 hour. Than (NE)—N—{[(E)-piperidine-1-carbonyl]imino}piperidine-1-carboxamide (1.7 g, 6.8 mmol, 2.0 eq.) was added the mixture was stirred at 25° C. for 60 mins under N₂. The resulting solution was diluted with 80 mL of water and extracted with EtOAc (80 mL×3). The combined organic layers were washed with brine (30 mL×2), dried over Na₂SO₄ and concentrated under reduced pressure. The residue was purified by flash column chromatography on silica gel (100-200 mesh silica gel, 0-10% EtOAc in PE) to give 2-(7-fluoro-2-iodobenzofuran-4-yl)acetonitrile (500 mg, yield 48.5%) as a yellow solid. ¹H NMR (400 MHz, DMSO-d₆) δ 7.48 (d, J=2.8 Hz, 1H), 7.27-7.18 (m, 2H), 4.22 (s, 2H).

### Step D: methyl 4-cyano-4-(7-fluoro-2-iodobenzofuran-4-yl)butanoate

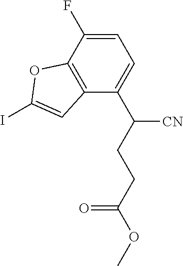 [SMILES: COC(=O)CCC(C#N)c1ccc(F)c2oc(I)cc12]

A mixture of 2-(7-fluoro-2-iodobenzofuran-4-yl)acetonitrile (500 mg, 1.6 mmol, 1.0 eq.), K₂CO₃ (275 mg, 2 mmol, 1.2 eq.) and methyl 3-bromopropanoate (332 mg, 2 mmol, 1.2 eq.) in DMF (5 mL). The mixture was stirred at 50° C. for 2 hours under N₂. LCMS showed the reaction was completed. The resulting solution was diluted with 100 mL of water and extracted with EtOAc (40 mL×3). The combined organic layers were washed with brine (40 mL×2), dried over Na₂SO₄ and concentrated under reduced pressure. The residue was purified by flash column chromatography on silica gel (100-200 mesh silica gel, 0-25% EtOAc in PE) to give methyl 4-cyano-4-(7-fluoro-2-iodobenzofuran-4-yl)butanoate (200 mg, yield 31%) as a yellow solid. ¹H NMR (400 MHz, DMSO-d₆) δ 7.53 (d, J=2.6 Hz, 1H), 7.30-7.19 (m, 2H), 4.57 (t, J=7.4 Hz, 1H), 3.96-4.12 (m, 2H), 2.40 (t, J=7.3 Hz, 2H), 2.05-2.20 (m, 2H), 1.16 (t, J=7.1 Hz, 3H)

### Step E: 3-(7-fluoro-2-iodobenzofuran-4-yl)piperidine-2,6-dione

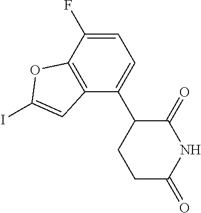 [SMILES: O=C1CCC(c2ccc(F)c3oc(I)cc23)C(=O)N1]

To a solution of methyl 4-cyano-4-(7-fluoro-2-iodobenzofuran-4-yl)butanoate (300 mg, 774 μmol, 1.0 eq.) in con. H₂SO₄ (0.4 mL) and ACOH (8 mL). The mixture was stirred at 100° C. for 2 hours under N₂. The resulting solution was diluted with 40 mL of water and extracted with EtOAc (40 mL×3). The combined organic layers were washed with brine (40 mL), dried over Na₂SO₄ and concentrated under reduced pressure. The residue was purified by flash column chromatography on silica gel (100-200 mesh silica gel, 0-35% EtOAc in PE) to give 3-(7-fluoro-2-iodobenzofuran-4-yl)piperidine-2,6-dione (45 mg, yield 16%) as a yellow solid.

LC-MS (ESI): mass calcd. for C₁₃H₉FINO₃, 372.9; m/z found, 373.9 [M+H]⁺.

### Intermediate 27. 5-chloro-1,3-dimethyl-7-(tetrahydro-2H-pyran-4-yl)-1,8-naphthyridin-2(1H)-one

### Step A: 5-chloro-7-(3,6-dihydro-2H-pyran-4-yl)-1,3-dimethyl-1,8-naphthyridin-2(1H)-one

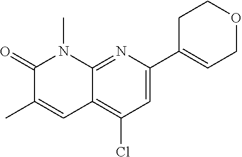 [SMILES: Cc1cc2c(Cl)cc(C3=CCOCC3)nc2n(C)c1=O]

To a solution of 5,7-dichloro-1,3-dimethyl-1,8-naphthyridin-2(1H)-one (1 g, 4.1 mmol, 1.0 eq.). 2-(3,6-dihydro-2H-pyran-4-yl)-4,4,5,5-tetramethyl-1,3,2-dioxaborolane (907 mg, 4.3 mmol, 1.1 eq.) and K₂CO₃ (1.7 g, 12.3 mmol, 3.0 eq.) in 1,4-Dioxane: H₂O(100 mL, 10:1) was added P, 1,1′-Bis(diphenylphosphino)ferrocene-palladium(II) dichloride (301 mg, 0.4 mmol, 0.1 eq.) at 25° C. The mixture was stirred at 80° C. for 18 hours under N₂ atmosphere. The reaction was monitored by LCMS. The resulting solution was diluted with 30 ml of water and extracted with EtOAc (3*30 mL). The combined organic layers were washed with brine, dried over anhydrous Na₂SO₄ and concentrated under reduced pressure. The residue was purified by flash column chromatography on silica gel (100-200 mesh silica gel, 0-30% EtOAc in PE) to afford 5-chloro-7-(3,6-dihydro-2H-pyran-4-yl)-1,3-dimethyl-1,8-naphthyridin-2(1H)-one (780 mg, yield 65%) as a white solid. LC-MS (ESI): mass calcd. for C₁₅H₁₅ClN₂O₂, 290.1; m/z found, 291.7 [M+H]⁺.

### Step B: 5-chloro-1,3-dimethyl-7-(tetrahydro-2H-pyran-4-yl)-1,8-naphthyridin-2(1H)-one

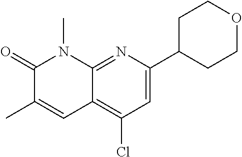 [SMILES: Cc1cc2c(Cl)cc(C3CCOCC3)nc2n(C)c1=O]

To a solution of 5,7-dichloro-1,3-dimethyl-1,8-naphthyridin-2(1H)-one (780 mg, 2.7 mmol, 1.0 eq.) in 2,2,2-Trifluoroethanol (7 mL) was added Wilkinson's catalyst (745 mg, 0.8 mmol, 0.3 eq.) at 25° C. The mixture was stirred at 60° C. for 18 hours under H₂ atmosphere. The reaction was monitored by LCMS. The resulting solution was diluted with 30 ml of water and extracted with EtOAc (3*30 mL). The mixture was filtered and the filtrate was concentrated under vacuum and purified by flash column chromatography on silica gel (100-200 mesh silica gel, 0-20% EtOAc in PE) to afford 5-chloro-1,3-dimethyl-7-(tetrahydro-2H-pyran-4-yl)-1,8-naphthyridin-2(1H)-one (500 mg, yield 64%) as a white solid. LC-MS (ESI): mass calcd. for C₁₅H₁₇ClN₂O₂, 292.1; m/z found, 293.4 [M+H]⁺.

### Intermediate 28. 3-(5-bromofuro[2,3-c]pyridin-3-yl)piperidine-2,6-dione

### Step A: methyl 5-bromo-3-oxo-2,3-dihydrofuro[2,3-c]pyridine-2-carboxylate

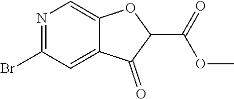

To a solution of methyl 2-bromo-5-fluoroisonicotinate (9 g, 38.5 mmol, 1.0 eq.) in DMF (110 mL) was added methyl 2-hydroxyacetate (6.9 g, 76.9 mmol, 2.0 eq.) and Cs₂CO₃ (25 g, 76.9 mmol, 2.0 eq.). The reaction mixture was stirred at 80° C. for 16 hours. LCMS showed the reaction was completed. The resulting solution was diluted with 100 mL of water and extracted with EtOAc (100 mL×3). The combined organic layers were washed with brine (50 mL), dried over Na₂SO₄ and concentrated under reduced pressure. The residue was purified by flash column chromatography on silica gel (100-200 mesh silica gel, 0-80% EtOAc in PE) to give methyl 5-bromo-3-oxo-2,3-dihydrofuro[2,3-c]pyridine-2-carboxylate (1.8 g, 17% yield) as a yellow solid.

LC-MS (ESI): mass calcd. for C₉H₆BrNO₄, 270.95; m/z found, 272.2 274.2[M+H]⁺.

### Step B: 5-bromofuro[2,3-c]pyridin-3(2H)-one

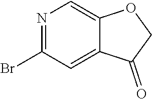 [SMILES: O=C1COc2cnc(Br)cc21]

To a solution of methyl 5-bromo-3-oxo-2,3-dihydrofuro[2,3-c]pyridine-2-carboxylate (1.8 g, 6.62 mmol, 1.0 eq.) in sulfuric acid (20 mL, w.t. =50%) and stirred at 70° C. for 16 hours. LCMS showed the reaction was completed. The reaction was poured into ice water (10 mL) and extracted with EtOAc (20*3 mL). The combined organic layers were washed with brine (10 mL), dried over Na₂SO₄ and concentrated under reduced pressure. The residue was purified by flash column chromatography on silica gel (100-200 mesh silica gel, 0-80% EtOAc in PE) to give 5-bromofuro[2,3-c]pyridin-3(2H)-one (500 mg, 35% yield) as a colorless oil.

LC-MS (ESI): mass calcd. for C₇H₄BrNO₂, 212.94; m/z found, 214.2 216.2 [M+H]⁺.

### Step C: ethyl 2-(5-bromofuro[2,3-c]pyridin-3-yl)acetate

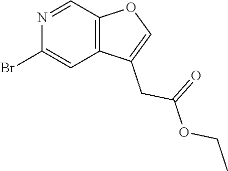 [SMILES: CCOC(=O)Cc1coc2cnc(Br)cc12]

To a solution of 5-bromofuro[2,3-c]pyridin-3(2H)-one (500 mg, 2.3 mmol, 1.0 eq.) and ethyl 2-(triphenyl-15-phosphaneylidene)acetate (977 mg, 2.8 mmol, 1.2 eq.) in Toluene (10 mL) and stirred at 110° C. for 16 hours. TLC showed the reaction was completed. The reaction was concentrated in vacuum and purified by flash column chromatography on silica gel (100-200 mesh silica gel, 0-50% EtOAc in PE) to give methyl 2-(5-bromofuro[2,3-c]pyridin-3-yl) acetate (430 mg, 65% yield) as a white solid. LC-MS (ESI): mass calcd. for C₁₁H₁₀BrNO₃, 282.98; m/z found, 294.3 296.3 [M+H]⁺.

### Step D: 3-(5-bromofuro[2,3-c]pyridin-3-yl)piperidine-2,6-dione

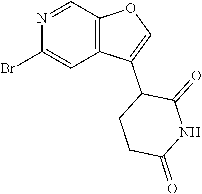 [SMILES: O=C1CCC(c2coc3cnc(Br)cc23)C(=O)N1]

To a solution of methyl 2-(5-bromofuro[2,3-c]pyridin-3-yl) acetate (200 mg, 741 μmol, 1.0 eq.) in DMF (5 mL) stirred at 25° C. was added t-BuOK (100 mg, 889 μmol, 1.2 eq.) and acrylamide (63 mg, 889 μmol, 1.2 eq.). The reaction was stirred at 25° C. for 1 hour. LCMS showed the starting materials was consumed and desired MS was formed. The reaction was extracted with EtOAc and water. The combined organic layer was washed with brine (10 mL), dried over anhydrous sodium sulfate, concentrated and purified by flash column chromatography on silica gel (100-200 mesh silica gel, 0-70% EtOAc in PE) to give 3-(5-bromofuro[2,3-c]pyridin-3-yl)piperidine-2,6-dione (100 mg, 43.7% yield) as a yellow solid.

LC-MS (ESI): mass calcd. for C₁₂H₉BrN₂O₃, 307.98 309.98; m/z found, 309.2 311.2 [M+H]⁺.

### Intermediate 29. Methyl 5-(5-((tert-butoxycarbonyl)(methyl)amino)pyrimidin-2-yl)-3-isopropylpicolinate

### Step A: methyl 5-(5-((tert-butoxycarbonyl)(methyl)amino)pyrimidin-2-yl)-3-chloropicolinate

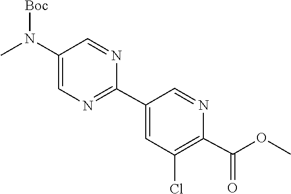

To a solution of tert-butyl (2-bromopyrimidin-5-yl)(methyl)carbamate (1 g, 3.5 mmol, 1.0 eq.), (5-chloro-6-(methoxycarbonyl)pyridin-3-yl)boronic acid (748 mg, 3.5 mmol, 1.0 eq.) and K₃PO₄ (2.1 g, 10.4 mmol, 3.0 eq.) in 1,4-Dioxane: H₂O(10 mL: 1 mL) was added Pd(dppf)Cl₂ (254 mg, 347 μmol, 0.1 eq.) at 25° C. The mixture was stirred at 100° C. for 6 hours under N₂ atmosphere. The resulting solution was diluted with 50 mL of water and extracted with EtOAc (3×50 mL). The combined organic layers were washed with brine, dried over anhydrous Na₂SO₄ and concentrated under reduced pressure. The residue was purified by flash column chromatography on silica gel (100-200 mesh silica gel, 0-25% EtOAc in PE) to afford methyl 5-(5-((tert-butoxycarbonyl)(methyl)amino)pyrimidin-2-yl)-3-chloropicolinate (770 mg, 59% yield) as a yellow solid. LC-MS (ESI): mass calcd. for C₁₇H₁₉ClN₄O₄, 378.1; m/z found, 379.1 [M+H]⁺.

### Step B: methyl 5-(5-((tert-butoxycarbonyl)(methyl)amino)pyrimidin-2-yl)-3-(prop-i-en-2-yl)picolinate

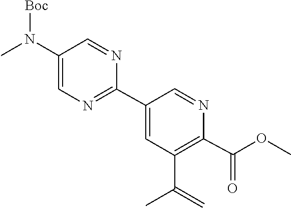

To a solution of methyl 5-(5-((tert-butoxycarbonyl)(methyl)amino)pyrimidin-2-yl)-3-chloropicolinate (770 mg, 2.0 mmol, 1.0 eq.), potassium trifluoro(prop-1-en-2-yl)borate (1.5 g, 10.2 mmol, 5.0 eq.), Pd(dppf)Cl₂ (149 mg, 203 μmol, 0.1 eq.) in 1,4-DMF: H₂O(10 mL: 0.5 mL) was added K₂CO₃ (848 mg, 6.1 mmol, 3.0 eq.) at 25° C. The mixture was stirred at 100° C. for 6 hours under N₂ atmosphere. The resulting solution was diluted with 50 mL of water and extracted with EtOAc (3×40 mL). The combined organic layers were washed with brine, dried over anhydrous Na₂SO₄ and concentrated under reduced pressure. The residue was purified by flash column chromatography on silica gel (100-200 mesh silica gel, 0-25% EtOAc in PE) to give methyl 5-(5-((tert-butoxycarbonyl)(methyl)amino)pyrimidin-2-yl)-3-(prop-i-en-2-yl)picolinate (160 mg, 21% yield) as a yellow solid. LC-MS (ESI): mass calcd. for C₂₀H₂₄N₄O₄, 384.2; m/z found, 385.2 [M+H]⁺.

### Step C: methyl 5-(5-((tert-butoxycarbonyl)(methyl)amino)pyrimidin-2-yl)-3-isopropylpicolinate

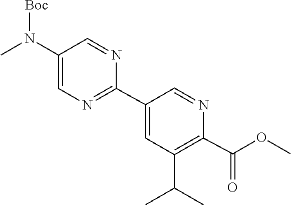

To a solution of 5-(5-((tert-butoxycarbonyl)(methyl)amino)pyrimidin-2-yl)-3-(prop-1-en-2-yl)picolinate (160 mg, 416 μmol, 1 eq.) in MeOH (5 mL) was added Pd/C (40 mg, w.t.=10%). The mixture was degassed with H₂ for 3 times and stirred at 25° C. using a H₂ balloon for 16 hours under H₂ atmosphere. The mixture was filtered through a Celite pad and the filtrate was concentrated under vacuum to afford methyl 5-(5-((tert-butoxycarbonyl)(methyl)amino)pyrimidin-2-yl)-3-isopropylpicolinate (160 mg, 99% yield) as a yellow solid. LC-MS (ESI): mass calcd. for C₂₀H₂₆N₄O₄, 386.2; m/z found, 387.2 [M+H]⁺.

### Intermediate 30. 1-(2-Bromobenzofuran-4-yl)-3-azabicyclo[3.1.1]heptane-2,4-dione

### Step A: 2-(benzofuran-4-yl)acetonitrile

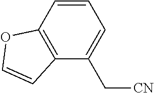 [SMILES: N#CCc1cccc2occc12]

To a solution of 4-bromobenzofuran (8.9 g, 45.2 mmol, 1.0 eq.), potassium 2-cyanoacetate (8.9 g, 72.3 mmol, 1.6 eq.) and S-Phos (3.7 g, 9 mmol, 0.2 eq.) in dry mesitylene (8 mL) was added Allylpalladium(II)chloridedimer (3.3 g, 9 mmol, 0.2 eq.) at 25° C. The mixture was stirred at 140° C. for 16 hours under N₂ atmosphere. The resulting solution was diluted with 200 mL of water and extracted with EtOAc (3×200 mL). The combined organic layers were washed with brine, dried over anhydrous Na₂SO₄ and concentrated under reduced pressure. The residue was purified by flash column chromatography on silica gel (100-200 mesh silica gel, 0-40% EtOAc in PE) to give 2-(benzofuran-4-yl)acetonitrile (3.3 g, 47% yield) as a yellow oil. No MS signal. ¹H NMR (400 MHz, DMSO-d₆) δ 8.07 (d, J=2.2 Hz, 1H), 7.60 (d, J=8.2 Hz, 1H), 7.35 (t, J=7.8 Hz, 1H), 7.27 (d, J=7.4 Hz, 1H), 7.15-7.11 (m, 1H), 4.29 (s, 2H).

### Step B: 1-(benzofuran-4-yl)-3,3-dimethoxycyclobutane-1-carbonitrile

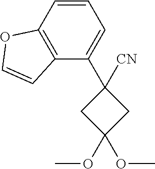 [SMILES: COC1(OC)CC(C#N)(c2cccc3occc23)C1]

To a solution of 2-(benzofuran-4-yl)acetonitrile (2.6 g, 16.5 mmol, 1.0 eq.), 1,3-dibromo-2,2-diethoxypropane (7.2 g, 24.8 mmol, 1.5 eq.) in dry DMF (30 mL) was added NaH (1.2 g, 29.8 mmol, 1.8 eq., w.t.=60% in oil) at 0° C. The mixture was stirred at 60° C. for 16 hours under N₂ atmosphere. The resulting solution was diluted with 100 mL of sat. NH₄Cl solution and extracted with EtOAc (3×100 mL). The combined organic layers were washed with brine, dried over anhydrous Na₂SO₄ and concentrated under reduced pressure. The residue was purified by flash column chromatography on silica gel (100-200 mesh silica gel, 0-30% EtOAc in PE) to give 1-(benzofuran-4-yl)-3,3-dimethoxycyclobutane-1-carbonitrile (2 g, 47% yield) as a yellow solid. No MS signal. ¹H NMR (400 MHz, CDCl3) δ 7.64 (d, J=2.2 Hz, 1H), 7.44 (d, J=8.3 Hz, 1H), 7.24 (t, J=7.9 Hz, 1H), 7.14 (d, J=7.5 Hz, 1H), 6.86 (d, J=1.4 Hz, 1H), 3.26 (s, 3H), 3.13 (d, J=2.4 Hz, 1H), 3.10 (s, 4H), 2.85 (d, J=2.1 Hz, 1H), 2.83 (d, J=2.4 Hz, 1H).

### Step C: 1-(benzofuran-4-yl)-3-oxocyclobutane-1-carbonitrile

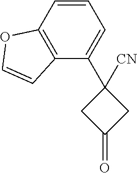 [SMILES: N#CC1(c2cccc3occc23)CC(=O)C1]

To a solution of 1-(benzofuran-4-yl)-3,3-dimethoxycyclobutane-1-carbonitrile (2 g, 7.8 mmol, 1.0 eq.) in Acetone (20 mL) was added TsOH (739 mg, 3.9 mmol, 0.5 eq.) at 25° C. The mixture was stirred at 90° C. for 16 hours under N₂ atmosphere. The resulting solution was diluted with 100 mL of water and extracted with EtOAc (3×50 mL). The combined organic layers were washed with brine, dried over anhydrous Na₂SO₄ and concentrated under reduced pressure. The residue was purified by flash column chromatography on silica gel (100-200 mesh silica gel, 0-30% EtOAc in PE) to give 1-(benzofuran-4-yl)-3-oxocyclobutane-1-carbonitrile (1.4 g, 85% yield) as a yellow solid. No MS signal.

### Step D: 1-(benzofuran-4-yl)-3-(methoxymethylene)cyclobutane-1-carbonitrile

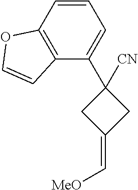 [SMILES: COC=C1CC(C#N)(c2cccc3occc23)C1]

To a solution of 1-(benzofuran-4-yl)-3-oxocyclobutane-1-carbonitrile (1 g, 4.7 mmol, 1.0 eq.) in MeOH (10 mL) was added Ohira-Bestmann Reagent (1.3 g, 7.1 mmol, 1.5 eq.) and K₂CO₃ (1.3 g, 9.5 mmol, 2.0 eq.) at 25° C. The mixture was stirred at 25° C. for 2 hours under N₂ atmosphere. The resulting solution was diluted with 20 ml of water and extracted with EtOAc (3×20 mL). The combined organic layers were washed with brine, dried over anhydrous Na₂SO₄ and concentrated under reduced pressure. The residue was purified by flash column chromatography on silica gel (100-200 mesh silica gel, 0-10% EtOAc in PE) to give 1-(benzofuran-4-yl)-3-(methoxymethylene)cyclobutane-1-carbonitrile (360 mg, 32% yield) as a yellow oil. No Ms signal.

### Step E: 1-(benzofuran-4-yl)-3-formylcyclobutane-1-carbonitrile

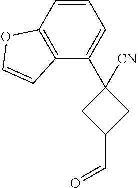 [SMILES: N#CC1(c2cccc3occc23)CC(C=O)C1]

A solution of 1-(benzofuran-4-yl)-3-(methoxymethylene)cyclobutane-1-carbonitrile (360 mg, 1.5 mmol, 1.0 eq.) in DCM (5 mL): TFA (1 mL) was stirred at 25° C. for 2 hours under N₂ atmosphere. The mixture was concentrated under reduced pressure to give 1-(benzofuran-4-yl)-3-formylcyclobutane-1-carbonitrile (330 mg, yield 97%) as a yellow oil. The product was used for next step directly without purification. No Ms signal.

### Step F: 3-(benzofuran-4-yl)-3-cyanocyclobutane-1-carboxylic acid

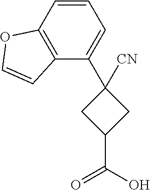 [SMILES: N#CC1(c2cccc3occc23)CC(C(=O)O)C1]

A mixture of 1-(5-bromobenzofuran-3-yl)-3-formylcyclobutane-1-carbonitrile (330 mg, 1.1 mmol, 1.0 eq.), 2-methylbut-2-ene (1.7 mL, 16.3 mmol, 15.0 eq.), NaClO₂ (491 mg, 5.43 mmol, 5.0 eq.) and NaH2PO₄ (260 mg, 2.2 mmol, 2.0 eq.) in t-BuOH (4 mL): THF (2 mL): H₂O(2 mL) was stirred at 25° C. for 1 hour. The resulting solution was diluted with 50 mL of water and extracted with EtOAc (50 mL×3). The combined organic layers were washed with brine (50 mL), dried over Na₂SO₄ and concentrated under reduced pressure to give the crude 3-(5-bromobenzofuran-3-yl)-3-cyanocyclobutane-1-carboxylic acid (200 mg, 58% yield) as a yellow oil.

### Step G: methyl 3-(benzofuran-4-yl)-3-cyanocyclobutane-1-carboxylate

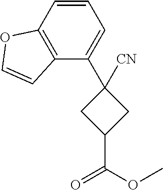 [SMILES: COC(=O)C1CC(C#N)(c2cccc3occc23)C1]

A mixture of 3-(benzofuran-4-yl)-3-cyanocyclobutane-1-carboxylic acid (200 mg, 829 μmol, 1.0 eq.) and H₂SO₄ (81 mg, 829 μmol, 1.0 eq.) in MeOH (10 mL) was stirred at 80° C. for 3 hours. The resulting solution was diluted with 50 mL of water and extracted with EtOAc (30 mL×3). The combined organic layers were washed with brine, dried over Na₂SO₄ and concentrated under reduced pressure. The residue was purified by flash column chromatography on silica gel (100-200 mesh silica gel, 0-10% EtOAc in PE) to give methyl 3-(benzofuran-4-yl)-3-cyanocyclobutane-1-carboxylate (200 mg, 95% yield) as a yellow solid. LC-MS (ESI): mass calcd. for C₁₅H₁₃NO₃, 255.1; m/z found, 254.1 [M−H]-.

### Step H: methyl 3-(benzofuran-4-yl)-3-carbamoylcyclobutane-1-carboxylate

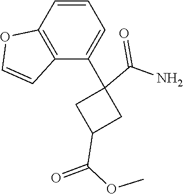 [SMILES: COC(=O)C1CC(C(N)=O)(c2cccc3occc23)C1]

To a solution of methyl 3-(benzofuran-4-yl)-3-cyanocyclobutane-1-carboxylate (200 mg, 783 μmol, 1.0 eq.) in Toluene (5 mL) was added Acetaldoxime (139 mg, 2.35 mmol, 3.0 eq.) and Indium trichloride (17 mg, 78.3 μmol, 0.1 eq.) at 25° C. The mixture was stirred at 100° C. for 1 hour. The resulting solution was diluted with 10 mL of water and extracted with EtOAc (3×10 mL). The combined organic layers were washed with brine, dried over anhydrous Na₂SO₄ and concentrated under reduced pressure. The residue was purified by flash column chromatography on silica gel (100-200 mesh silica gel, 0-90% EtOAc in PE) to give methyl 3-(benzofuran-4-yl)-3-carbamoylcyclobutane-1-carboxylate (100 mg, 47% yield) as a white solid. LC-MS (ESI): mass calcd. for C₁₅H₁₅NO₄, 273.1; m/z found, 274.1 [M+H]⁺.

### Step I: 1-(benzofuran-4-yl)-3-azabicyclo[3.1.1]heptane-2,4-dione

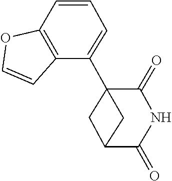 [SMILES: O=C1NC(=O)C2(c3cccc4occc34)CC1C2]

To a solution of methyl 3-(benzofuran-4-yl)-3-carbamoylcyclobutane-1-carboxylate (100 mg, 366 μmol, 1.0 eq.) in MeCN (5 mL) was added Triton B (184 mg, 1.1 mmol, 3.0 eq.) at 25° C. The mixture was stirred at 25° C. for 1 hour. The resulting solution was diluted with 10 mL of water and extracted with EtOAc (3×10 mL). The combined organic layers were washed with brine, dried over anhydrous Na₂SO₄ and concentrated under reduced pressure. The residue was purified by flash column chromatography on silica gel (100-200 mesh silica gel, 0-90% EtOAc in PE) to give 1-(benzofuran-4-yl)-3-azabicyclo[3.1.1]heptane-2,4-dione (80 mg, 91% yield) as a white solid. LC-MS (ESI): mass calcd. for C₁₄H₁₁NO₃, 241.1; m/z found, 242.1 [M+H]⁺.

### Step J: 1-(2-bromobenzofuran-4-yl)-3-azabicyclo[3.1.1]heptane-2,4-dione

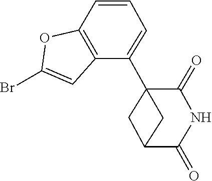 [SMILES: O=C1NC(=O)C2(c3cccc4oc(Br)cc34)CC1C2]

To a solution of 1-(benzofuran-4-yl)-3-azabicyclo[3.1.1]heptane-2,4-dione (80 mg, 332 μmol, 1.0 eq.) in MeCN (5 mL) was added NBS (177 mg, 332 μmol, 3.0 eq.) and cat. DMF (two drops) at 50° C. The mixture was stirred at 50° C. for 16 hours. The resulting solution was purified by flash column chromatography on silica gel (100-200 mesh silica gel, 0-70% EtOAc in PE) to give 1-(2-bromobenzofuran-4-yl)-3-azabicyclo[3.1.1]heptane-2,4-dione (20 mg, 18% yield) as a white solid. LC-MS (ESI): mass calcd. for C₁₄H₁₀BrNO₃, 319.0; m/z found, 320.0/322.0 [M+H]⁺.

### Intermediate 31. 5-(5-(6,8-dimethyl-2-morpholino-7-oxo-7,8-dihydropyrido[2,3-d]pyrimidin-4-yl)-5,6,7,8-tetrahydropyrido[3,2-d]pyrimidin-2-yl)picolinic acid

### Step A: 4-chloro-6-(methylamino)-2-(methylthio)pyrimidine-5-carbaldehyde

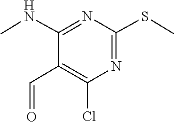

To a solution of 4,6-dichloro-2-(methylthio)pyrimidine-5-carbaldehyde (6 g, 26.9 mmol, 1.0 eq.) in THF (60 mL)was added methanamine (5 mL, 32.3 mmol, 1 M in THF, 1.2 eq.) under N₂. The solution was stirred at 70° C. for 2 hours to give yellow solution. LCMS showed the reaction was completed. The solution was concentrated. The residue was purified by flash chromatography (100-200 mesh silica gel, 0% ˜50% EtOAC in PE) to afford 4-chloro-6-(methylamino)-2-(methylthio)pyrimidine-5-carbaldehyde (3.3 g, yield 54%) as a yellow solid.

LC-MS (ESI): mass calcd. for C₇H₈ClN₃OS, 217.7; m/z found, 218.7[M+H]⁺.

### Step B: 4-hydroxy-6,8-dimethyl-2-(methylthio)pyrido[2,3-d]pyrimidin-7(8H)-one

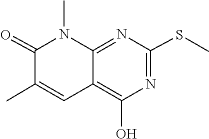 [SMILES: CSc1nc(O)c2cc(C)c(=O)n(C)c2n1]

To a solution of 4-chloro-6-(methylamino)-2-(methylthio)pyrimidine-5-carbaldehyde (3.3 g, 20.7 mmol, 1.0 eq.) in DMF (50 mL) was added TEA (8.64 mL, 62 mmol, 3.0 eq.), propionyl chloride (3.8 g, 41.3 mmol, 2.0 eq.) and DMAP (2.5 g, 21 mmol, 1.0 eq.) at 0° C.

The solution was stirred at 90° C. for 16 hours to give yellow solution. LCMS showed the reaction was completed. The solution was filtered and concentrated to afford 4-hydroxy-6,8-dimethyl-2-(methylthio)pyrido[2,3-d]pyrimidin-7(8H)-one (1.4 g, yield 27%) as a yellow solid. LC-MS (ESI): mass calcd. for C₁₀H₁₁N₃O₂S, 237.3 m/z found, 238.3[M+H]⁺.

### Step C: 6,8-dimethyl-2-(methylthio)-7-oxo-7,8-dihydropyrido[2,3-d]pyrimidin-4-yl trifluoromethanesulfonate

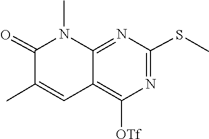 [SMILES: CSc1nc(OS(=O)(=O)C(F)(F)F)c2cc(C)c(=O)n(C)c2n1]

To a solution of 4-hydroxy-6,8-dimethyl-2-(methylthio)pyrido[2,3-d]pyrimidin-7(8H)-one (1.4 g, 5.9 mmol, 1.0 eq.) in DCM (20 mL) was added Tf₂O (1.9 mL, 11.8 mmol, 2.0 eq.) and DIEA (2.0 mL, 11.8 mmol, 2.0 eq.) at 0° C. The solution was stirred at 0° C. for 1 hour to give yellow solution. LCMS showed the reaction was completed. The solution was quenched water, extracted with DCM (100 mL) and the layers were separated. The organic layer was washed with brine (100 mL), dried over Na₂SO₄, filtered and concentrated. The residue was purified by flash chromatography (100-200 mesh silica gel, 0% ˜20% EtOAC in PE) to afford 6,8-dimethyl-2-(methylthio)-7-oxo-7,8-dihydropyrido[2,3-d]pyrimidin-4-yl trifluoromethanesulfonate (1.4 g, yield 61%) as a white solid. LC-MS (ESI): mass calcd. for C₁₁H₁₀F₃N₃O₄S², 369.3 m/z found, 370.3 [M+H]⁺.

### Step D: 4-(2-chloro-7,8-dihydropyrido[3,2-d]pyrimidin-5(6H)-yl)-6,8-dimethyl-2-(methylthio)pyrido[2,3-d]pyrimidin-7(8H)-one

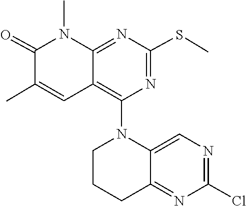 [SMILES: CSc1nc(N2CCCc3nc(Cl)ncc32)c2cc(C)c(=O)n(C)c2n1]

To a solution of 2-chloro-5,6,7,8-tetrahydropyrido[3,2-d]pyrimidine (140 mg, 825 μmol, 1.0 eq.) in 1,4-Dioxane (10 mL) was added 6,8-dimethyl-2-(methylthio)-7-oxo-7,8-dihydropyrido[2,3-d]pyrimidin-4-yl trifluoromethanesulfonate (305 mg, 825 μmol, 1.0 eq.), Cs₂CO₃ (807 mg, 2.5 mmol, 3.0 eq.), BINAP (103 mg, 165 μmol, 0.2 eq.)and Pd₂(dba)₃ (75.6 mg, 82.5 μmol, 0.1 eq.) under N₂. The solution was stirred at 100° C. for 2 hours to give yellow solution.

LCMS showed the reaction was completed. The solution was filtered and concentrated. The residue was purified by flash chromatography (100-200 mesh silica gel, 0% ˜100% EtOAC in PE) to afford 4-(2-chloro-7,8-dihydropyrido[3,2-d]pyrimidin-5(6H)-yl)-6,8-dimethyl-2-(methylthio)pyrido[2,3-d]pyrimidin-7(8H)-one (75 mg, yield 22%) as a yellow solid.LC-MS (ESI): mass calcd. for C₁₇H₁₇ClN₆OS, 388.3; m/z found, 389.3 [M+H]⁺.

### Step E: 4-(2-chloro-7,8-dihydropyrido[3,2-d]pyrimidin-5(6H)-yl)-6,8-dimethyl-2-(methylsulfonyl)pyrido[2,3-d]pyrimidin-7(8H)-one

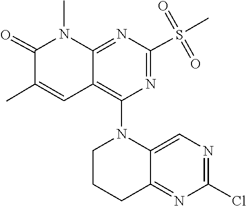 [SMILES: Cc1cc2c(N3CCCc4nc(Cl)ncc43)nc(S(C)(=O)=O)nc2n(C)c1=O]

To a solution of 4-(2-chloro-7,8-dihydropyrido[3,2-d]pyrimidin-5(6H)-yl)-6,8-dimethyl-2-(methylthio)pyrido[2,3-d]pyrimidin-7(8H)-one (75 mg, 193 μmol, 1.0 eq). in DCM (10 mL) was added 3-Chloroperoxybenzoic acid (100 mg, 579 μmol, 3.0 eq.). The solution was stirred at 25° C. for 30 mins to give yellow solution. LCMS showed the reaction was completed.

The solution was quenched with sat. Na₂S²O₃ solution, extracted with DCM (50 mL) and the layers were separated. The organic layer was washed with brine (50 mL), dried over Na₂SO₄, filtered and concentrated. The residue was purified by flash chromatography (100-200 mesh silica gel, 0% ˜100% EtOAC in PE) to afford 4-(2-chloro-7,8-dihydropyrido[3,2-d]pyrimidin-5(6H)-yl)-6,8-dimethyl-2-(methylsulfonyl)pyrido[2,3-d]pyrimidin-7(8H)-one (58 mg, yield 68%) as a white solid. LC-MS (ESI): mass calcd. for C₁₇H₁₇ClN₆O₃S, 420.8; m/z found, 421.8 [M+H]⁺.

### Step F: 4-(2-chloro-7,8-dihydropyrido[3,2-d]pyrimidin-5(6H)-yl)-6,8-dimethyl-2-morpholinopyrido[2,3-d]pyrimidin-7(8H)-one

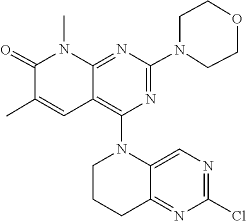 [SMILES: Cc1cc2c(N3CCCc4nc(Cl)ncc43)nc(N3CCOCC3)nc2n(C)c1=O]

To a solution of 4-(2-chloro-7,8-dihydropyrido[3,2-d]pyrimidin-5(6H)-yl)-6,8-dimethyl-2-(methylsulfonyl)pyrido[2,3-d]pyrimidin-7(8H)-one (58 mg, 138 μmol, 1.0 eq.) in MeCN (5 mL)was added DIEA (48 μL, 276 μmol, 2.0 eq.) and morpholine (24 mg, 276 μmol, 2.0 eq.) under N₂. The solution was stirred at 80° C. for 2 hours to give yellow solution. LCMS showed the reaction was completed. The solution was concentrated. The product residue was purified by flash chromatography (100-200 mesh silica gel, 0% ˜100% EtOAC in PE) to afford 4-(2-chloro-7,8-dihydropyrido[3,2-d]pyrimidin-5(6H)-yl)-6,8-dimethyl-2-morpholinopyrido[2,3-d]pyrimidin-7(8H)-one (50 mg, yield 81%) as a yellow solid. LC-MS (ESI): mass calcd. for C₂₀H₂₂ClN₇O₂, 427.9; m/z found, 428.9 [M+H]⁺.

### Step G: methyl 5-(5-(6,8-dimethyl-2-morpholino-7-oxo-7,8-dihydropyrido[2,3-d]pyrimidin-4-yl)-5,6,7,8-tetrahydropyrido[3,2-d]pyrimidin-2-yl)picolinate

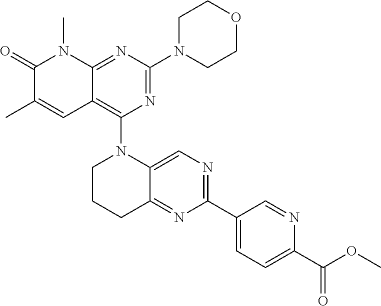

To a solution of methyl 5-(4,4,5,5-tetramethyl-1,3,2-dioxaborolan-2-yl)picolinate (61.5 mg, 234 μmol, 2.0 eq.) in 1,4-Dioxane (2 mL) and Water (0.2 mL) was added 4-(2-chloro-7,8-dihydropyrido[3,2-d]pyrimidin-5(6H)-yl)-6,8-dimethyl-2-morpholinopyrido[2,3-d]pyrimidin-7(8H)-one (50 mg, 117 μmol, 1.0 eq.), K₃PO₄ (74.4 mg, 351 μmol, 3.0 eq.) and Pd(dtbpf)Cl₂ (15.2 mg, 23.4 μmol, 0.2 eq.) under N₂. The solution was stirred at 100° C. for 2 hours to give black solution. LCMS showed the reaction was completed. The solution was quenched water, extracted with EA (50 mL) and the layers were separated. The organic layer was washed with brine (50 mL), dried over Na₂SO₄, filtered and concentrated. The residue was purified by flash chromatography (100-200 mesh silica gel, 0% ˜100% EtOAC in PE) to afford methyl 5-(5-(6,8-dimethyl-2-morpholino-7-oxo-7,8-dihydropyrido[2,3-d]pyrimidin-4-yl)-5,6,7,8-tetrahydropyrido[3,2-d]pyrimidin-2-yl)picolinate (55 mg, yield 85%) as a yellow solid. LC-MS (ESI): mass calcd. for C₂₇H₂₈N₈O₄, 528.6; m/z found, 529.6 [M+H]⁺.

### Step H: 5-(5-(6,8-dimethyl-2-morpholino-7-oxo-7,8-dihydropyrido[2,3-d]pyrimidin-4-yl)-5,6,7,8-tetrahydropyrido[3,2-d]pyrimidin-2-yl)picolinic acid

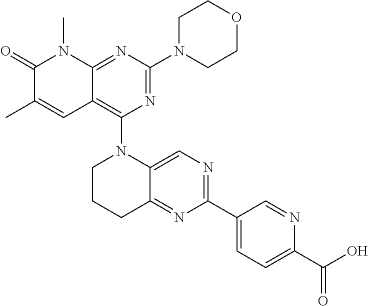 [SMILES: Cc1cc2c(N3CCCc4nc(-c5ccc(C(=O)O)nc5)ncc43)nc(N3CCOCC3)nc2n(C)c1=O]

To a solution of methyl 5-(5-(6,8-dimethyl-2-morpholino-7-oxo-7,8-dihydropyrido[2,3-d]pyrimidin-4-yl)-5,6,7,8-tetrahydropyrido[3,2-d]pyrimidin-2-yl)picolinate (50 mg, 94.6 μmol, 1.0 eq.) in THF (5 mL) and Water (1 mL) was added LiOH.H₂O(7.9 mg, 189 mol, 2.0 eq.). The solution was stirred at 25° C. for 30 mins to give yellow solution. LCMS showed the reaction was completed. The solution was concentrated to afford 5-(5-(6,8-dimethyl-2-morpholino-7-oxo-7,8-dihydropyrido[2,3-d]pyrimidin-4-yl)-5,6,7,8-tetrahydropyrido[3,2-d]pyrimidin-2-yl)picolinic acid (50 mg, crude) as a yellow solid. LC-MS (ESI): mass calcd. for C₂₆H₂₆N₈O₄, 514.6; m/z found, 515.6 [M+H]⁺.

### Intermediate 32. 1,3-Dimethyl-2-oxo-7-(1-propionylpiperidin-4-yl)-1,2-dihydroquinolin-5-yl trifluoromethanesulfonate

### Step A: tert-butyl 4-(5-methoxy-1,3-dimethyl-2-oxo-1,2-dihydroquinolin-7-yl)-3,6-dihydropyridine-1(2H)-carboxylate

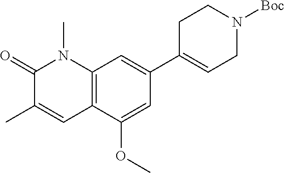 [SMILES: COc1cc(C2=CCN(C(=O)OC(C)(C)C)CC2)cc2c1cc(C)c(=O)n2C]

To a solution of 7-bromo-5-methoxy-1,3-dimethylquinolin-2(1H)-one (500 mg, 1.8 mmol, 1 eq.) in 1,4-Dioxane: H₂O=10:1 (11 mL) was added tert-butyl 4-(4,4,5,5-tetramethyl-1,3,2-dioxaborolan-2-yl)-3,6-dihydropyridine-1(2H)-carboxylate (1.1 g, 3.5 mmol, 2 eq.) and Pd(dppf)Cl₂ (130 mg, 177 μmol, 0.1 eq.) and K₂CO₃ (735 mg, 5.3 mmol, 3 eq.) at 25° C. The mixture was then stirred at 100° C. for 4 hours under N₂. LCMS showed the reaction was completed. The resulting solution was diluted with 50 mL of water, then extracted with EtOAc (30 mL×3). The combined organic layers were washed with brine (30 mL), dried over Na₂SO₄ and concentrated under reduced pressure. The residue was purified by flash column chromatography on silica gel (100-200 mesh silica gel, 0-30% EtOAc in PE) to give tert-butyl 4-(5-methoxy-1,3-dimethyl-2-oxo-1,2-dihydroquinolin-7-yl)-3,6-dihydropyridine-1(2H)-carboxylate (500 mg, 73.4% yield) as a yellow oil. LC-MS (ESI): mass calcd. for C₂₂H₂₈N₂O₄, 384; m/z found, 385 [M+H]⁺.

### Step B: tert-butyl 4-(5-methoxy-1,3-dimethyl-2-oxo-1,2-dihydroquinolin-7-yl)piperidine-1-carboxylate

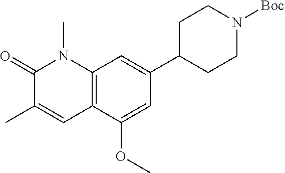 [SMILES: COc1cc(C2CCN(C(=O)OC(C)(C)C)CC2)cc2c1cc(C)c(=O)n2C]

To a solution of tert-butyl 4-(5-methoxy-1,3-dimethyl-2-oxo-1,2-dihydroquinolin-7-yl)-3,6-dihydropyridine-1(2H)-carboxylate (500 mg, 1.3 mmol, 1 eq.) in MeOH (30 mL) was added Pd/C (100 mg, 130 μmol, 0.1 eq. w.t.=10%) at 25° C. The mixture was then stirred at 30° C. for 16 hours under H₂. LCMS showed the reaction was completed. The resulting solution was diluted with 20 ml of MeOH and filtered. The combined organic layers were concentrated under reduced pressure. The residue was purified with flash column chromatography on silica gel (EtOAc in PE, from 0% to 20%) to afford tert-butyl 4-(5-methoxy-1,3-dimethyl-2-oxo-1,2-dihydroquinolin-7-yl)piperidine-1-carboxylate (500 mg, 99% yield) as a bright oil.

LC-MS (ESI): mass calcd. for C₂₂H₃MN₂O₄, 386; m/z found, 387 [M+H]⁺.

### Step C: 5-methoxy-1,3-dimethyl-7-(piperidin-4-yl)quinolin-2(1H)-one

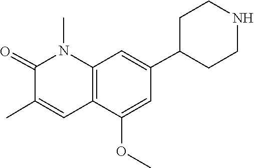 [SMILES: COc1cc(C2CCNCC2)cc2c1cc(C)c(=O)n2C]

To a solution of tert-butyl 4-(5-methoxy-1,3-dimethyl-2-oxo-1,2-dihydroquinolin-7-yl)piperidine-1-carboxylate (500 mg, 1.3 mmol, 1 eq.) in DCM: TFA=5:1 (12 mL) was added at 0° C. The mixture was then stirred at 25° C. for 1 hour under N₂. LCMS showed the reaction was completed. The reaction mixture was concentrated to give 5-methoxy-1,3-dimethyl-7-(piperidin-4-yl)quinolin-2(1H)-one (320 mg, 86% yield) as a yellow solid.

LC-MS (ESI): mass calcd. for C₁₇H₂₂N₂O₂, 286; m/z found, 287 [M+H]⁺.

### Step D: 5-methoxy-1,3-dimethyl-7-(1-propionylpiperidin-4-yl)quinolin-2(1H)-one

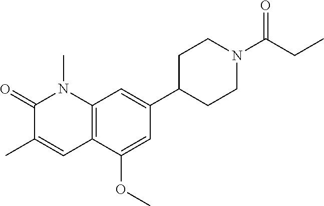 [SMILES: CCC(=O)N1CCC(c2cc(OC)c3cc(C)c(=O)n(C)c3c2)CC1]

To a stirred solution of 5-methoxy-1,3-dimethyl-7-(piperidin-4-yl)quinolin-2(1H)-one (320 mg, 1.1 mmol, 1 eq.) was added dropwise TEA (311 μL, 2.2 mmol, 2 eq.) and propionyl chloride (155 mg, 1.7 mmol, 1.5 eq.) in DCM (10 mL) at 0° C. under N₂. The reaction mixture was stirred overnight at room temperature under N₂ atmosphere. The resulting solution was diluted with 50 mL of H₂O and extracted with EtOAc (20 mL×3). The combined organic layers were washed with brine, dried over Na₂SO₄, and concentrated under reduced pressure. The residue was purified by flash column chromatography on silica gel (100-200 mesh silica gel, 0-50% EtOAc in PE) to give the 5-methoxy-1,3-dimethyl-7-(1-propionylpiperidin-4-yl)quinolin-2(1H)-one (300 mg, 78.4% yield) as a yellow solid.

LC-MS (ESI): mass calcd. for C₂₀H₂₆N₂O₃, 342; m/z found, 343 [M+H]⁺.

### Step E: 5-hydroxy-1,3-dimethyl-7-(1-propionylpiperidin-4-yl)quinolin-2(1H)-one

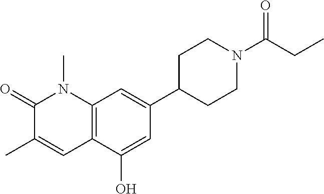 [SMILES: CCC(=O)N1CCC(c2cc(O)c3cc(C)c(=O)n(C)c3c2)CC1]

To a stirred solution of 5-methoxy-1,3-dimethyl-7-(1-propionylpiperidin-4-yl)quinolin-2(1H)-one (300 mg, 876 μmol, 1 eq.) was added dropwise BBr3 (248 μL, 2.6 mmol, 3 eq.) in DCM (10 mL) at 0° C. under N₂ atmosphere. The reaction mixture was stirred overnight at room temperature under N₂ atmosphere. The resulting solution was diluted with 50 mL of H₂O and extracted with EtOAc (20 mL×3). The combined organic layers were washed with brine, dried over Na₂SO₄, and concentrated under reduced pressure. The residue was purified by flash column chromatography on silica gel (100-200 mesh silica gel, 0-50% EtOAc in PE) to give 5-hydroxy-1,3-dimethyl-7-(1-propionylpiperidin-4-yl)quinolin-2(1H)-one (240 mg, 83.4% yield) as a yellow solid. LC-MS (ESI): mass calcd. for C₁₉H₂₄N₂O₃, 328; m/z found, 329 [M+H]+

### Step F: 1,3-dimethyl-2-oxo-7-(1-propionylpiperidin-4-yl)-1,2-dihydroquinolin-5-yl trifluoromethanesulfonate

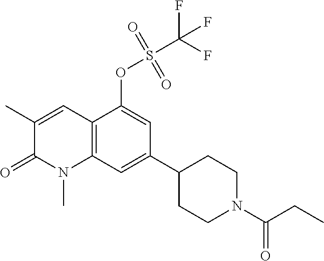 [SMILES: CCC(=O)N1CCC(c2cc(OS(=O)(=O)C(F)(F)F)c3cc(C)c(=O)n(C)c3c2)CC1]

To a stirred solution of 5-hydroxy-1,3-dimethyl-7-(1-propionylpiperidin-4-yl)quinolin-2(1H)-one (200 mg, 609 μmol, 1 eq.) was added dropwise Et3N (255 μL, 1.8 mmol, 3 eq.) and Phenyl triflimide (435 mg, 1.2 mmol, 2 eq.) in DCM (10 mL) at 0° C. under N₂ atmosphere. The reaction mixture was stirred overnight at room temperature under N₂ atmosphere. The mixture was diluted with water and extracted with DCM. The combined organic layers were washed with brine, dried over anhydrous Na₂SO₄ and concentrated under reduced pressure. The residue was purified by flash chromatography (eluent, 0-10%ethyl acetate in petroleum ether/0˜10% methanol in dichloromethane) to give the 1,3-dimethyl-2-oxo-7-(1-propionylpiperidin-4-yl)-1,2-dihydroquinolin-5-yl trifluoromethanesulfonate (200 mg, 71.3% yield) as a yellow solid.

LC-MS (ESI): mass calcd. for C₂₀H₂₃F₃N₂O₅S, 460; m/z found, 461 [M+H]+

### Intermediate 33. 8-Bromo-3-chloro-5-methylisoquinoline

### Step A: N-(2-bromo-5-methylbenzyl)-2,2-diethoxyacetimidamide

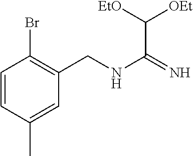 [SMILES: CCOC(OCC)C(=N)NCc1cc(C)ccc1Br]

To a solution of (2-bromo-5-methylphenyl)methanamine (600 mg, 3 mmol, 1.0 eq.) in MeOH (10 mL) was added DIEA (1.6 mL, 9 mmol, 3.0 eq.) and methyl 2,2-diethoxyacetimidate (580 mg, 3.6 mmol, 1.2 eq.) under N₂. The solution was stirred at 70° C. for 3 hours to give yellow solution. LCMS showed the reaction was completed. The solution was quenched water, extracted with DCM (100 mL) and the layers were separated. The organic layer was washed with brine (100 mL), dried over Na₂SO₄, filtered and concentrated to afford N-(2-bromo-5-methylbenzyl)-2,2-diethoxyacetimidamide (800 mg, yield 77%) as a yellow oil. LC-MS (ESI): mass calcd. for C₁₄H₂₁BrN₂O₂, 328.2/330.2; m/z found, 329.2/331.2 [M+H]⁺.

### Step B: 8-bromo-5-methylisoquinolin-3-amine

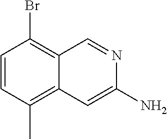

A solution of N-(2-bromo-5-methylbenzyl)-2,2-diethoxyacetimidamide (2.9 g, 8.8 mmol, 1.0 eq.) in H₂SO₄ (30 mL) was stirred at 80° C. for 6 hours to give brown solution. LCMS showed the reaction was completed. The solution was adjusted to pH=12 with NaOH solution, The organic layer was filtered and concentrated to afford 8-bromo-5-methylisoquinolin-3-amine (1.5 g, yield 68%) as a yellow solid. LC-MS (ESI): mass calcd. for C₁₀H₉BrN₂, 236.1/238.1; m/z found, 237.1/239.1 [M+H]⁺.

### Step C: 8-bromo-3-chloro-5-methylisoquinoline

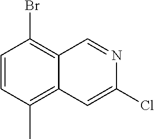

To a solution of 8-bromo-5-methylisoquinolin-3-amine (1.5 g, 6.3 mmol, 1.0 eq.) in CHCl₃ (20 mL) was added benzyl(triethyl)ammonium chloride (5.8 g, 25.3 mmol, 4.0 eq.) and tert-Butyl nitrite (1.5 mL, 12.7 mmol, 2.0 eq.) under N₂. The solution was stirred at 50° C. for 16 hours to give yellow solution. LCMS showed the reaction was completed. The solution was quenched NaHCO₃ solution, extracted with DCM (60 mL) and the layers were separated. The organic layer was washed with brine (60 mL), dried over Na₂SO₄, filtered and concentrated. The residue was purified by flash chromatography (100-200 mesh silica gel, 0% ˜30% EtOAC in PE) to afford 8-bromo-3-chloro-5-methylisoquinoline (160 mg, yield 9.4%) as a yellow solid. LC-MS (ESI): mass calcd. for C₁₀H₇BrClN, 255.1/257.1; m/z found, 256.1/258.1 [M+H]⁺.

### Intermediate 34. Methyl 5′-((tert-butoxycarbonyl)(methyl)amino)-6-methyl-[2,2′-bipyrazine]-5-carboxylate

### Step A: tert-butyl methyl(5-(tributylstannyl)pyrazin-2-yl)carbamate

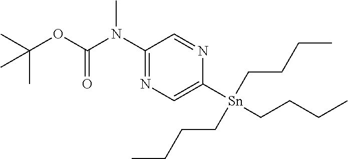 [SMILES: CCC[CH2][Sn]([CH2]CCC)([CH2]CCC)[c]1cnc(N(C)C(=O)OC(C)(C)C)cn1]

To a solution of tert-butyl (5-bromopyrazin-2-yl)(methyl)carbamate (900 mg, 3.1 mmol, 1.0 eq.) in 1,4-Dioxane (10mL) was added Pd(PPh₃)₄ (722 mg, 625 μmol, 0.2 eq.), LiCl (397 mg, 9.4 mmol, 3.0 eq.) and Sn₂Bu₆ (2.4 mL, 4.7 mmol, 1.5 eq.) under N₂. The solution was stirred at 120° C. for 16 hours to give black solution. LCMS showed the reaction was completed. The resulting solution was diluted with 100 mL of water and extracted with EtOAc (3×100 mL). The combined organic layers were washed with brine (3×100 mL), dried over Na₂SO₄, and concentrated under reduced pressure. The residue was purified by flash chromatography (100-200 mesh silica gel, 0% ˜2% EtOAC in PE) to afford tert-butyl methyl(5-(tributylstannyl)pyrazin-2-yl)carbamate (250 mg, yield 15%) as a colorless oil. LC-MS (ESI): mass calcd. for C₂₂H₄₁N₃O₂Sn, 499.3; m/z found, 500.3 [M+H]⁺.

### Step B: methyl 5′-((tert-butoxycarbonyl)(methyl)amino)-6-methyl-[2,2′-bipyrazine]-5-carboxylate

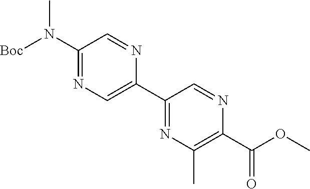 [SMILES: COC(=O)c1ncc(-c2cnc(N(C)C(=O)OC(C)(C)C)cn2)nc1C]

To a solution of tert-butyl methyl(5-(tributylstannyl)pyrazin-2-yl)carbamate (200 mg, 401 μmol, 1.0 eq.) in 1,4-Dioxane (10 mL) was added Pd(PPh₃)₄ (93 mg, 85 μmol, 0.2 eq.) and methyl 5-chloro-3-methylpyrazine-2-carboxylate (75 mg, 401 μmol, 1.0 eq.) under N₂. The solution was stirred at 110° C. for 8 hours to give yellow solution. LCMS showed the reaction was completed. The resulting solution was diluted with 50 mL of water and extracted with EtOAc (3×50 mL). The combined organic layers were washed with brine (50 mL), dried over Na₂SO₄, and concentrated under reduced pressure. The residue was purified by flash chromatography (100-200 mesh silica gel, 0% ˜50% EtOAC in PE) to afford methyl 5′-((tert-butoxycarbonyl)(methyl)amino)-6-methyl-[2,2′-bipyrazine]-5-carboxylate (100 mg, yield 66%) as a yellow solid. LC-MS (ESI): mass calcd. for C₁₇H₂₁N₅O₄, 359.4; m/z found, 360.4 [M+H]⁺.

### Intermediate 35. 3-(8-bromo-2,3,4,5-tetrahydro-1H-benzo[b]azepin-1-yl)piperidine-2,6--dione

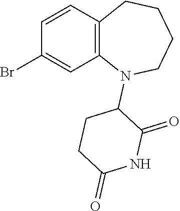 [SMILES: O=C1CCC(N2CCCCc3ccc(Br)cc32)C(=O)N1]

To a solution of 8-bromo-2,3,4,5-tetrahydro-1H-benzo[b]azepine (500 mg, 2.2 mmol, 1 eq.) in 1,4-Dioxane (2 mL) was added 3-bromopiperidine-2,6-dione (849 mg, 4.4 mmol, 2 eq.) and DIEA (770 μL, 4.4 mmol, 2 eq.) at 25° C. The mixture was then stirred at 80° C. for 16 hours under N₂ atmosphere. The resulting solution was diluted with 30 mL of water and extracted with EtOAc (30 mL×3). The combined organic layers were washed with brine, dried over Na₂SO₄, and concentrated under reduced pressure. The residue was purified by flash column chromatography on silica gel (100-200 mesh silica gel, 0-30% EtOAc in PE) to give 3-(8-bromo-2,3,4,5-tetrahydro-1H-benzo[b]azepin-1-yl)piperidine-2,6-dione (150 mg, 20% yield) as a white solid. LC-MS (ESI): mass calcd. for C₁₅H₁₇BrN₂O₂, 336.0/338.0; m/z found, 337.2/339.2 [M+H]⁺.

### Intermediate 36. 5-bromo-1,3-dimethyl-7-(2-oxa-6-azaspiro[3.3]heptan-6-yl)quinolin-2(1H)-one

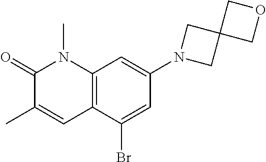 [SMILES: Cc1cc2c(Br)cc(N3CC4(COC4)C3)cc2n(C)c1=O]

To a solution of 5-bromo-1,3-dimethyl-2-oxo-1,2-dihydroquinolin-7-yl trifluoromethanesulfonate (100 mg, 250 μmol, 1.0 eq.) in Toluene (3 mL) was added Pd₂(dba)₃ (23 mg, 25.0 μmol, 0.1 eq.), Xant-Phos (29 mg, 50.0 μmol, 0.2 eq.), 2-oxa-6-azaspiro[3.3]heptane (25 mg, 250 μmol, 1.0 eq.) and Cs₂CO₃ (163 mg, 500 μmol, 2.0 eq.). The mixture was stirred at 90° C. for 3 hours under N₂ atmosphere. The resulting solution was diluted with 10 mL of H₂O and extracted with EtOAc (8 mL×3). The combined organic layers were washed with brine, dried over Na₂SO₄, and concentrated under reduced pressure. The residue was purified by flash column chromatography on silica gel (100-200 mesh silica gel, 0-50% EtOAc in PE) to give 5-bromo-1,3-dimethyl-7-(2-oxa-6-azaspiro[3.3]heptan-6-yl)quinolin-2(1H)-one (40 mg, yield 46%) as a yellow solid. LC-MS (ESI): mass calcd. for C₁₆H₁₇BrN₂O₂, 348.0/350.0; m/z found,349.1/351.1 [M+H]⁺.

### Intermediate 37. tert-butyl (3-(4-(2,6-dioxopiperidin-3-yl)-3-fluorobenzofuran-2-yl)prop-2-yn-1-yl)carbamate

### Step A -J: 2-(3-fluoro-2-iodobenzofuran-4-yl)acetonitrile

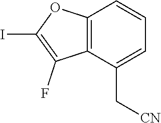 [SMILES: N#CCc1cccc2oc(I)c(F)c12]

2-(3-fluoro-2-iodobenzofuran-4-yl)acetonitrile was prepared from 1-bromo-3-fluoro-2-iodobenzene to the procedures described in SKT-0018150, Steps A -J. 2-(3-fluoro-2-iodobenzofuran-4-yl)acetonitrile (500 mg, 0.02% yield over 10 steps) was obtained as a yellow solid. LC-MS (ESI): mass calcd. for C₁₀H₅FINO, 300.9; m/z found, 301.9 [M H]+.

### Step K: tert-butyl (3-(4-(cyanomethyl)-3-fluorobenzofuran-2-yl)prop-2-yn-1-yl)carbamate

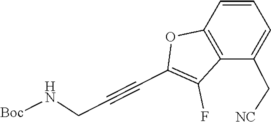 [SMILES: CC(C)(C)OC(=O)NCC#Cc1oc2cccc(CC#N)c2c1F]

A mixture of 2-(3-fluoro-2-iodobenzofuran-4-yl)acetonitrile (300 mg, 996.5 μmol, 1.0 eq.), tert-butyl prop-2-yn-1-ylcarbamate (464 mg, 2.9 mmol, 3.0 eq.), CuI (38 mg, 199 μmol, 0.2 eq.), TEA (1.1 mL, 8 mmol, 8.0 eq.) and Pd(PPh₃)Cl₂ (139 mg, 199 μmol, 0.2 eq.) in DMF (5 mL). The mixture was stirred at 25° C. for 4 hours under nitrogen. The mixture was diluted water and extracted with EtOAC (50 mL×3). The combined organic layers were washed with brine (50 mL), dried over Na₂SO₄, filtered and concentrated. The residue was purified by flash chromatography (100-200 mesh silica gel, 0-30% EtOAc in PE) to afford tert-butyl (3-(4-(cyanomethyl)-3-fluorobenzofuran-2-yl)prop-2-yn-1-yl)carbamate (260 mg, yield 79%) as a yellow solid. LC-MS (ESI): mass calcd. for C₁₈H₁₇FN₂O₃, 328.1; m/z found, 329.1 [M H]+.

### Step L: methyl 4-(2-(3-((tert-butoxycarbonyl)amino)prop-1-yn-1-yl)-3-fluorobenzofuran-4-yl)-4-cyanobutanoate

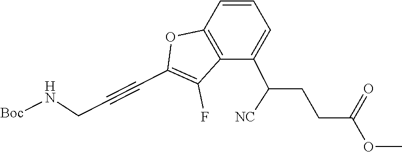 [SMILES: COC(=O)CCC(C#N)c1cccc2oc(C#CCNC(=O)OC(C)(C)C)c(F)c12]

To a solution of tert-butyl (3-(4-(cyanomethyl)-3-fluorobenzofuran-2-yl)prop-2-yn-1-yl)carbamate (260 mg., 792 μmol, 1.0 eq.) in DMF (5 mL) was added K₂CO₃ (328 mg, 2.3 mmol, 3.0 eq.) and methyl acrylate (68 mg, 792 μmol, 1.0 eq.) at 25° C. The mixture was then stirred at 50° C. for 1 hour under nitrogen. The mixture was diluted water and extracted with EtOAC (50 mL×3). The combined organic layers were washed with brine (50 mL), dried over Na₂SO₄, filtered and concentrated. The residue was purified by flash chromatography (100-200 mesh silica gel, 0-40% EtOAc in PE) to afford methyl 4-(2-(3-((tert-butoxycarbonyl)amino)prop-1-yn-1-yl)-3-fluorobenzofuran-4-yl)-4-cyanobutanoate (220 mg, yield 67%) as a yellow solid. LC-MS (ESI): mass calcd. for C₂₂H₂₃FN₂O₅, 414.1; m/z found, 415.1 [M H]+.

### Step M: methyl 5-amino-4-(2-(3-((tert-butoxycarbonyl)amino)prop-1-yn-1-yl)-3-fluorobenzofuran-4-yl)-5-oxopentanoate

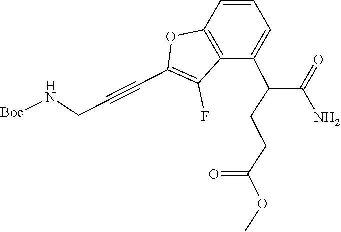 [SMILES: COC(=O)CCC(C(N)=O)c1cccc2oc(C#CCNC(=O)OC(C)(C)C)c(F)c12]

To a solution of methyl 4-(2-(3-((tert-butoxycarbonyl)amino)prop-1-yn-1-yl)-3-fluorobenzofuran-4-yl)-4-cyanobutanoate (220 mg, 530.8 μmol, 1.0 eq.) in Toluene (8 mL) was added indium (III) chloride (12 mg, 53.1 μmol, 0.1 eq.) and (E)-acetaldehyde oxime (94 mg, 1.5 mmol, 3.0 eq.) at 25° C. with stirring. The mixture was stirred at 110° C. for 2 hours. The mixture was diluted water and extracted with EtOAC (50 mL×3). The combined organic layers were washed with brine (50 mL), dried over Na₂SO₄, filtered and concentrated. The residue was purified by flash chromatography (100-200 mesh silica gel, 0-40% EtOAc in PE) to give methyl 5-amino-4-(2-(3-((tert-butoxycarbonyl)amino)prop-1-yn-1-yl)-3-fluorobenzofuran-4-yl)-5-oxopentanoate (90 mg, yield 39%) as a white solid. LC-MS (ESI): mass calcd. for C₂₂H₂₅FN₂O₆, 432.1; m/z found, 433.1 [M H]*.

### Step N: tert-butyl (3-(4-(2,6-dioxopiperidin-3-yl)-3-fluorobenzofuran-2-yl)prop-2-yn-1-yl)carbamate

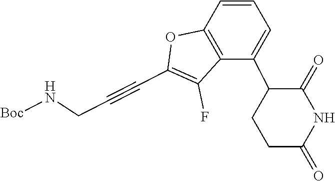 [SMILES: CC(C)(C)OC(=O)NCC#Cc1oc2cccc(C3CCC(=O)NC3=O)c2c1F]

To a mixture of methyl 5-amino-4-(2-(3-((tert-butoxycarbonyl)amino)prop-1-yn-1-yl)-3-fluorobenzofuran-4-yl)-5-oxopentanoate (70 mg, 162 μmol, 1.0 eq.) in THF (8 mL) was added t-BuOK (36 mg, 324 μmol, 2.0 eq.) under nitrogen and stirred at room temperature for 30 mins. The mixture was diluted water and extracted with EtOAC (50 mL×3). The combined organic layers were washed with brine (50 mL), dried over Na₂SO₄, filtered and concentrated. The residue was purified by flash chromatography (100-200 mesh silica gel, 0-40% EtOAc in PE) to afford tert-butyl (3-(4-(2,6-dioxopiperidin-3-yl)-3-fluorobenzofuran-2-yl)prop-2-yn-1-yl)carbamate (30 mg, yield 44%) as a yellow solid LC-MS (ESI): mass calcd. for C₂₁H₂₁FN₂O₅, 400.1; m/z found, 401.1 [M H]⁺.

### Intermediate 38. 5-Chloro-1,3-dimethyl-7-morpholino-1,8-naphthyridin-2(1H)-one-5-chloro-1,3-dimethyl-7-morpholino-1,8-naphthyridin-2(1H)-one

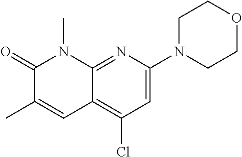 [SMILES: Cc1cc2c(Cl)cc(N3CCOCC3)nc2n(C)c1=O]

To the solution of 5,7-dichloro-1,3-dimethyl-1,8-naphthyridin-2(1H)-one (500 mg, 2.0 mmol, 1 eq.) and TBAI (152 mg, 411 μmol, 0.2 eq.) in DMF (20 mL) was added Morpholine (269 mg, 269 μL, 3.0 mmol, 1.5 eq.) at 25° C. The mixture was stirred at 50° C. for 12 hours under N₂ atmosphere. The resulting solution was diluted with 20 mL of water and extracted with EtOAc (3*20 mL). The combined organic layers were washed with brine, dried over anhydrous Na₂SO₄ and concentrated under reduced pressure. The residue was purified by flash column chromatography on silica gel (100-200 mesh silica gel, 0-100% DCM in EtOAc) to afford 5-chloro-1,3-dimethyl-7-morpholino-1,8-naphthyridin-2(1H)-one (80.0 mg, yield 13.2%) as a yellow solid. ¹H NMR (400 MHz, DMSO-d₆) δ 7.76 (d, J=1.2 Hz, 1H), 6.98 (s, 1H), 3.73-3.69 (m, 4H), 3.67-3.63 (m, 4H), 3.61 (s, 3H), 2.11 (d, J=1.0 Hz, 3H).

### Intermediate 39. 6-Bromo-1-ethyl-3,4-dimethyl-1,3-dihydro-2H-benzo[d]imidazol-2-one

### Step A: 5-bromo-N-ethyl-3-methyl-2-nitroaniline

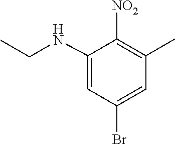

To a solution of 5-bromo-1-fluoro-3-methyl-2-nitrobenzene (1.5 g, 6.4 mmol, 1.0 eq.) and K₂CO₃ (3.5 g, 25.6 mmol, 4.0 eq.) in DMSO (20 mL) was added ethyl amine hydrochloride (1.1 g, 12.8 mmol, 2.0 eq.) at 25° C. The mixture was stirred at 120° C. for 2 hours under N₂ atmosphere. The reaction was monitored by LCMS. The resulting solution was diluted with 30 ml of water and extracted with EtOAc (3*30 mL). The combined organic layers were washed with brine, dried over anhydrous Na₂SO₄ and concentrated under reduced pressure. The residue was purified by flash column chromatography on silica gel (100-200 mesh silica gel, 0-10% EtOAc in PE) to afford 5-bromo-N-ethyl-3-methyl-2-nitroaniline (1.5 g, yield 90%) as a yellow solid. LC-MS (ESI): mass calcd. for C₉H₁₁BrN₂O₂, 258.0; m/z found, 259.1 [M+H]⁺.

### Step B: 5-bromo-N₁-ethyl-3-methylbenzene-1,2-diamine

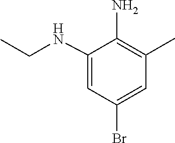

To a solution of 5-bromo-N-ethyl-3-methyl-2-nitroaniline (1.5 g, 5.8 mmol, 1.0 eq.) and NH₄Cl (3.1 g, 2.1 mmol, 10.0 eq.) in EtOH: H₂O(5:1, 24 mL) was added Iron (3.2 g, 57.9 mmol, 10.0 eq.) at 25° C. The mixture was stirred at 90° C. for 2 hours under N₂ atmosphere. The reaction was monitored by LCMS. The reaction was diluted with DCM: MeOH (10:1, 100 mL), and filtered through a pad of silica. The filter pad was washed with DCM: MeOH (10:1, 100 mL). The filtrates were concentrated under vacuum to give 5-bromo-N₁-ethyl-3-methylbenzene-1,2-diamine (1.2 g, yield 91%) as a brown solid. LC-MS (ESI): mass calcd. for C₉H₁₃BrN₂, 228.1; m/z found, 229.4 [M+H]⁺.

### Step C: 6-bromo-1-ethyl-4-methyl-1,3-dihydro-2H-benzo[d]imidazol-2-one

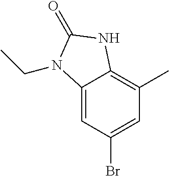

To a solution of 5-bromo-N1-ethyl-3-methylbenzene-1,2-diamine (1.2 g, 5.2 mmol, 1.0 eq.) in THF (15 mL) was added CDI (1.7 g, 10.5 mmol, 2.0 eq.) at 25° C. The mixture was stirred at 70° C. for 7 hours under N₂ atmosphere. The reaction was monitored by LCMS. The reaction solution was filtered through filter paper to give 6-bromo-1-ethyl-4-methyl-1,3-dihydro-2H-benzo[d]imidazol-2-one (1.2 g, crude) as a white solid. LC-MS (ESI): mass calcd. for C₁₀H₁₁BrN₂O, 254.1; m/z found, 255.4 [M+H]⁺.

### Step D: 6-bromo-1-ethyl-3,4-dimethyl-1,3-dihydro-2H-benzo[d]imidazol-2-one

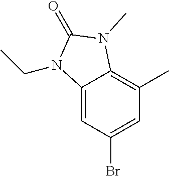

To a solution of 6-bromo-1-ethyl-4-methyl-1,3-dihydro-2H-benzo[d]imidazol-2-one (1.2 g, 4.7 mmol, 1.0 eq.) and Sodium hydride (1.0 g, 9.4 mmol, 2.0 eq., w.t. =60% in oil) in DMF (15 mL) was added Methyl iodide (0.5 mL, 7.1 mmol, 1.5 eq.) at 0° C. The mixture was stirred at 25° C. for 1 hour under N₂ atmosphere. The reaction was monitored by LCMS. The resulting solution was diluted with 30 ml of water and extracted with EtOAc (3*30 mL). The combined organic layers were washed with brine, dried over anhydrous Na₂SO₄ and concentrated under reduced pressure. The residue was purified by flash column chromatography on silica gel (100-200 mesh silica gel, 0-10% EtOAc in PE) to give 6-bromo-1-ethyl-3,4-dimethyl-1,3-dihydro-2H-benzo[d]imidazole-2-one (1.1 g, yield 87%) as a yellow solid. LC-MS (ESI): mass calcd. for C₁₁H₁₃BrN₂O, 268.0; m/z found, 269.1 [M+H]⁺.

### Intermediate 39. 3-Methyl-7-morpholino-2-oxo-2,3-dihydrobenzo[d]oxazol-5-yl trifluoromethanesulfonate

### Step A: 5-methoxy-3-methyl-7-morpholinobenzo[d]oxazol-2(3H)-one

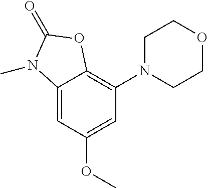 [SMILES: COc1cc(N2CCOCC2)c2oc(=O)n(C)c2c1]

To a flask containing 7-bromo-5-methoxy-3-methylbenzo[d]oxazol-2(3H)-one (700 mg, 2.71 mmol, 1 eq.), morpholine (473 mg, 5.42 mmol, 2 eq.), X-phos (129 mg, 271 μmol, 0.06 eq.) and Cs₂CO₃ (1.7 g, 5.42 mmol, 2 eq.) in 1,4-Dioxane (5 mL) was added Pd₂(dba)₃ (124 mg, 136 μmol, 0.05 eq.) at room temperature. The mixture was stirred at 100° C. for 6 hours under N₂ atmosphere. The reaction was monitored by LCMS. The resulting solution was diluted with 50 ml of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford 5-methoxy-3-methyl-7-morpholinobenzo[d]oxazol-2(3H)-one (600 mg, yield 84%) as a yellow solid. LC-MS (ESI): mass calcd. for C₁₃H₁₆N₂O₄, 264.1; m/z found, 265.2 [M+H]⁺.

### Step B: 5-hydroxy-3-methyl-7-morpholinobenzo[d]oxazol-2(3H)-one

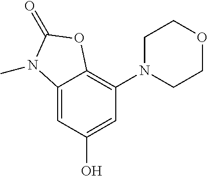 [SMILES: Cn1c(=O)oc2c(N3CCOCC3)cc(O)cc21]

To a solution of 5-methoxy-3-methyl-7-morpholinobenzo[d]oxazol-2(3H)-one (200 mg, 757 μmol, 1 eq.) in DCM (5 mL) was added BBr₃ (569 mg, 2.27 mmol, 3 eq.) at 0° C. The mixture was stirred at 25° C. for 2 hours under N₂ atmosphere. The reaction was monitored by LCMS. The mixture was dropwise added into MeOH (10 mL) and concentrated in vacuum. The residue was purified by flash column chromatography on silica gel (100-200 mesh silica gel, 0-50% EtOAc in PE) to afford 5-hydroxy-3-methyl-7-morpholinobenzo[d]oxazol-2(3H)-one (160 mg, yield 84%) as a white solid. LC-MS (ESI): mass calcd. for C₁₂H₁₄N₂O₄, 250.1; m/z found, 251.2 [M+H]⁺.

### Step C: 3-methyl-7-morpholino-2-oxo-2,3-dihydrobenzo[d]oxazol-5-yl trifluoromethanesulfonate

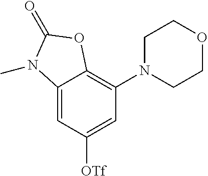 [SMILES: Cn1c(=O)oc2c(N3CCOCC3)cc(OS(=O)(=O)C(F)(F)F)cc21]

To a solution of 5-hydroxy-3-methyl-7-morpholinobenzo[d]oxazol-2(3H)-one (160 mg, 639 μmol, 1 eq.) and TEA (174 μL, 1.28 mmol, 2 eq.) in DCM (5 mL) was added Tf₂O (234 mg, 831 μmol, 1.3 eq.) at 0° C. The mixture was stirred at 25° C. for 2 hours under N₂ atmosphere. The reaction was monitored by LCMS. The resulting solution was diluted with 50 ml of water and extracted with EtOAc. The combined organic layers were washed with brine, dried over anhydrous Na₂SO₄ and concentrated under reduced pressure. The residue was purified by flash column chromatography on silica gel (100-200 mesh silica gel, 0-30% EtOAc in PE) to afford 3-methyl-7-morpholino-2-oxo-2,3-dihydrobenzo[d]oxazol-5-yl trifluoromethanesulfonate (170 mg, yield 70%) as a white solid. LC-MS (ESI): mass calcd. for C₁₃H₁₃F₃N₂O₆S, 382.0; m/z found, 383.1 [M+H]⁺.

### Intermediate 40. 6-Bromo-1,3-dimethyl-4-(tetrahydro-2H-pyran-4-yl)-1,3-dihydro-2H-benzo[d]imidazol-2-one

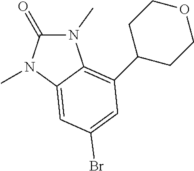 [SMILES: Cn1c(=O)n(C)c2c(C3CCOCC3)cc(Br)cc21]

To a solution of Zn (2.5 g, 38.8 mmol, 8 eq.),1,3-dioxoisoindolin-2-yl tetrahydro-2H-pyran-4-carboxylate (1.3 g, 4.9 mmol, 1 eq.),6-bromo-4-iodo-1,3-dimethyl-1,3-dihydro-2H-benzo[d]imidazol-2-one (1.8 g, 4.9 mmol, 1 eq.) and (biby)₂Ni2(u-Cl)₂Cl₂(H₂O₂)₂ (311 mg, 485 μmol, 0.1 eq.) in DMA (80 mL) was stirred at 0° C. Then TMSCl (1.6 g, 14.6 mmol, 3 eq.) was added to this mixture. The mixture was stirred at 0° C. for 1 hour under N₂ atmosphere. The resulting solution was diluted with 100 mL of water and extracted with EtOAc (100 mL×3). The combined organic layers were washed with brine, dried over Na₂SO₄, and concentrated under reduced pressure. The residue was purified by flash column chromatography on silica gel (100-200 mesh silica gel, 0-30% EtOAc in PE) to give 6-bromo-1,3-dimethyl-4-(tetrahydro-2H-pyran-4-yl)-1,3-dihydro-2H-benzo[d]imidazol-2-one (300 mg, 19% yield) as a white solid. LC-MS (ESI): mass calcd. for C₁₄H₁₇BrN₂O₂, 324.0; m/z found, 325.2 [M+H]⁺.

### Intermediate 41. 1-((3-iodophenyl)amino)-3-azabicyclo[3.1.1]heptane-2,4-dione

### Step A: methyl 3-cyano-3-((3-iodophenyl)amino)cyclobutane-1-carboxylate

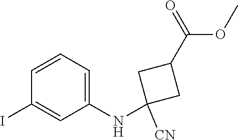 [SMILES: COC(=O)C1CC(C#N)(Nc2cccc(I)c2)C1]

To a solution of 3-iodoaniline (5.0 g, 22.8 mmol, 1 eq.) in EtOH (50 mL) was added methyl 3-oxocyclobutane-1-carboxylate (2.6 g, 20.5 mmol, 0.9 eq.) and trimethylsilanecarbonitrile (2.0 g, 20.5 mmol, 0.9 eq.). The reaction was stirred at 25° C. for 16 hours under N₂. The resulting solution was diluted with 50 mL of water and extracted with EtOAc (3* 100 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford methyl 3-cyano-3-((3-iodophenyl)amino)cyclobutane-1-carboxylate (6.0 g, yield 74%) as a white solid. LC-MS (ESI): mass calcd. for C₁₃H₁₃IN₂O₂, 356.0; m/z found, 357.0 [M+H]⁺.

### Step B: methyl 3-carbamoyl-3-((3-iodophenyl)amino)cyclobutane-1-carboxylate

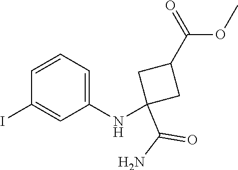 [SMILES: COC(=O)C1CC(Nc2cccc(I)c2)(C(N)=O)C1]

To a solution of methyl 3-cyano-3-((3-iodophenyl)amino)cyclobutane-1-carboxylate (3.0 g, 8.4 mmol, 1 eq.) in Toluene (25 mL) was added Indium trichloride (186 mg, 842 μmol, 0.1 eq.) and (E)-acetaldehyde oxime (1.5 g, 25.3 mmol, 3 eq.). The reaction was stirred at 110° C. for 1 hour under N₂. The resulting solution was concentrated under reduced pressure and purified by flash column chromatography on silica gel (100-200 mesh silica gel, 0-100% EtOAc in PE) to afford methyl 3-carbamoyl-3-((3-iodophenyl)amino)cyclobutane-1-carboxylate (1.8 g, yield 57.1%) as a yellow solid. LC-MS (ESI): mass calcd. for C₁₃H₁₅IN₂O₃, 374.0; m/z found, 375.0 [M+H]⁺.

### Step C: 1-((3-iodophenyl)amino)-3-azabicyclo[3.1.1]heptane-2,4-dione

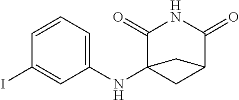 [SMILES: O=C1NC(=O)C2(Nc3cccc(I)c3)CC1C2]

To a solution of methyl 3-carbamoyl-3-((3-iodophenyl)amino)cyclobutane-1-carboxylate (1.8 g, 4.8 mmol, 1 eq.) in THF (20 mL) was added potassium 2-methylpropan-2-olate (1.6 g, 14.4 mmol, 3 eq.).The reaction was stirred at 25° C. for 1 hour under N₂ The resulting solution was diluted with 50 ml of water and extracted with EtOAc (3*60 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 1-((3-iodophenyl)amino)-3-azabicyclo[3.1.1]heptane-2,4-dione (280 mg, yield 17.0%) as a white solid. LC-MS (ESI): mass calcd. for C₁₂H₁₁IN₂O₂, 342.0; m/z found, 343.0 [M+H]⁺.

### Intermediate 43. 5-(difluoro(7-isopropyl-1,3-dimethyl-2-oxo-2,3-dihydro-1H-benzo[d]imidazol-5-yl)methyl)-[2,3′-bipyridine]-6′-carboxylic acid

### Step A: 6-(6-chloronicotinoyl)-4-isopropyl-1,3-dimethyl-1,3-dihydro-2H-benzo[d]imidazol-2-one

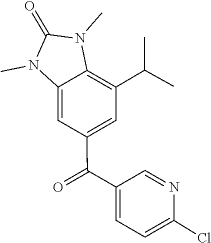 [SMILES: CC(C)c1cc(C(=O)c2ccc(Cl)nc2)cc2c1n(C)c(=O)n2C]

To a stirred mixture of (7-isopropyl-1,3-dimethyl-2-oxo-2,3-dihydro-1H-benzo[d]imidazol-5-yl)boronic acid (1.2 g, 5.0 mmol, 1.2 eq.) and Pd₂(dba)₃ (765 mg, 835 μmol, 0.2 eq.) in DMF (8.0 mL) and Et3N (1.6 mL) was added 2-chloro-5-iodopyridine (1.0 g, 4.2 mmol, 1 eq.) and K₂CO₃ (1.7 g, 12.5 mmol, 3 eq.), the reaction mixture was stirred at 100° C. for 12 hours under CO atmosphere. The resulting solution was diluted with 50 mL of water and extracted with EtOAc (3*80 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford the 6-(6-chloronicotinoyl)-4-isopropyl-1,3-dimethyl-1,3-dihydro-2H-benzo[d]imidazol-2-one (0.2 g, yield 14%) as a yellow solid. LC-MS (ESI): mass calcd. for C₁₈H₁₈ClN₃O₂, 343.1; m/z found, 344.1 [M+H]⁺.

### Step B: methyl 5-(7-isopropyl-1,3-dimethyl-2-oxo-2,3-dihydro-1H-benzo[d]imidazole-5-carbonyl)-[2,3′-bipyridine]-6′-carboxylate

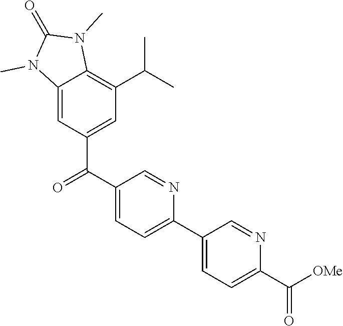 [SMILES: COC(=O)c1ccc(-c2ccc(C(=O)c3cc(C(C)C)c4c(c3)n(C)c(=O)n4C)cn2)cn1]

To a stirred mixture of 6-(6-chloronicotinoyl)-4-isopropyl-1,3-dimethyl-1,3-dihydro-2H-benzo[d]imidazol-2-one (80 mg, 233 μmol, 1 eq.) and methyl 5-(4,4,5,5-tetramethyl-1,3,2-dioxaborolan-2-yl)picolinate (35 mg, 256 μmol, 1.1 eq.) in 1,4-Dioxane (2 mL) and Water (200 μL) was added Pd₂(dba)₃ (43 mg, 46.5 μmol, 0.2 eq.) and K₂CO₃ (97 mg, 698 μmol, 3 eq.), the reaction mixture was stirred at 100° C. for 2 hours under the N₂ atmosphere. The resulting solution was diluted with 50 ml of water and extracted with EtOAc (3* 15 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford methyl 5-(7-isopropyl-1,3-dimethyl-2-oxo-2,3-dihydro-1H-benzo[d]imidazole-5-carbonyl)-[2,3′-bipyridine]-6′-carboxylate (60.0 mg, yield 58.0%) as a yellow solid. LC-MS (ESI): mass calcd. for C₂₅H₂₄N₄O₄, 444.2; m/z found, 445.2 [M+H]⁺.

### Step C: methyl 5-(difluoro(7-isopropyl-1,3-dimethyl-2-oxo-2,3-dihydro-1H-benzo[d]imidazol-5-yl)methyl)-[2,3′-bipyridine]-6′-carboxylate

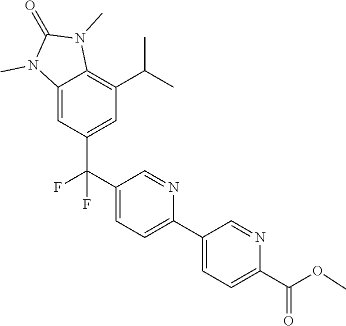

To a stirred mixture of methyl 5-(7-isopropyl-1,3-dimethyl-2-oxo-2,3-dihydro-1H-benzo[d]imidazole-5-carbonyl)-[2,3′-bipyridine]-6′-carboxylate (100 mg, 225 μmol, 1 eq.) in BAST (3 mL). The reaction mixture was stirred at 90° C. for 3 hours. The mixture was quenched with aq. NaHCO₃ (30 mL) and diluted with 30 ml of water and extracted with DCM (3*30 mL).

The combined organic layers were washed with brine, dried over anhydrous Na₂SO₄ and concentrated under reduced pressure. The residue was purified by flash column chromatography on silica gel (100-200 mesh silica gel, 0-20% EtOAc in PE) to afford methyl 5-(difluoro(7-isopropyl-1,3-dimethyl-2-oxo-2,3-dihydro-1H-benzo[d]imidazol-5-yl)methyl)-[2,3′-bipyridine]-6′-carboxylate (10.0 mg, yield 9.5%) as a yellow solid. LC-MS (ESI): mass calcd. for C₂₅H₂₄F₂N₄O₃, 466.2; m/z found, 467.2 [M+H]⁺.

### Step D: 5-(difluoro(7-isopropyl-1,3-dimethyl-2-oxo-2,3-dihydro-1H-benzo[d]imidazol-5-yl)methyl)-[2,3′-bipyridine]-6′-carboxylic acid

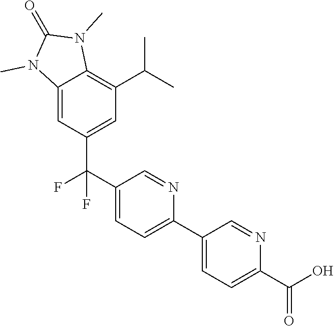 [SMILES: CC(C)c1cc(C(F)(F)c2ccc(-c3ccc(C(=O)O)nc3)nc2)cc2c1n(C)c(=O)n2C]

To the solution of methyl 5-(difluoro(7-isopropyl-1,3-dimethyl-2-oxo-2,3-dihydro-1H-benzo[d]imidazol-5-yl)methyl)-[2,3′-bipyridine]-6′-carboxylate (10 mg, 21.4 μmol, 1 eq.) in THF (3 mL) was added the solution of lithium hydroxide (770 g, 32.2 μmol, 1.5 eq.) in H₂O (750 μL) and stirred at 25° C. for 30 min. The reaction was monitored by LCMS. The resulting solution was dried over anhydrous sodium sulfate and concentrated under reduced pressure to afford the product 5-(difluoro(7-isopropyl-1,3-dimethyl-2-oxo-2,3-dihydro-1H-benzo[d]imidazol-5-yl)methyl)-[2,3′-bipyridine]-6′-carboxylic acid (10 mg, yield, crude) as a Li salt. LC-MS (ESI): mass calcd. for C₂₄H₂₂F₂N₄O₃, 452.2; m/z found, 453.1 [M+H]⁺.

### Intermediate 44. Methyl 5-(8-(7-chloro-1,3-dimethyl-2-oxo-1,2-dihydro-1,6-naphthyridin-5-yl)isoquinolin-3-yl)picolinate

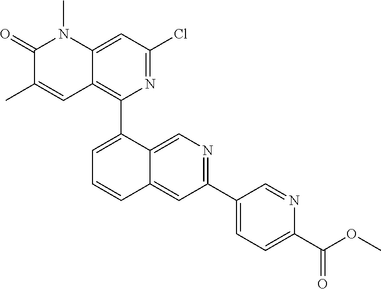

To a flask containing 5,7-dichloro-1,3-dimethyl-1,6-naphthyridin-2(1H)-one (1 g, 4.11 mmol, 1 eq.), methyl 5-(8-(4,4,5,5-tetramethyl-1,3,2-dioxaborolan-2-yl)isoquinolin-3-yl)picolinate (1.6 g, 4.11 mmol, 1 eq.) and K₂CO₃ (1.7 g, 12.3 mmol, 3 eq.) in 1,4-Dioxane (10 mL) and H₂O(1 mL) was added Pd(dppf)Cl₂ (602 mg, 823 μmol, 0.2 eq.) at 25° C. The mixture was stirred at 100° C. for 2 hours under N₂ atmosphere. The reaction was monitored by LCMS. The resulting solution was diluted with 50 ml of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford methyl 5-(8-(7-chloro-1,3-dimethyl-2-oxo-1,2-dihydro-1,6-naphthyridin-5-yl)isoquinolin-3-yl)picolinate (800 mg, yield 41%) as a brown solid. LC-MS (ESI): mass calcd. for C₂₆H₁₉ClN₄O₃, 471.1; m/z found, 472.2 [M+H]⁺.

### Intermediate 45. methyl 5-(5-(1,3-dimethyl-2-oxo-7-(tetrahydro-2H-pyran-4-yl)-1,2-dihydro-1,6-naphthyridin-5-yl)-5,6,7,8-tetrahydropyrido[3,2-d]pyrimidin-2-yl)picolinate

### Step A: methyl 5-(5-(7-(3,6-dihydro-2H-pyran-4-yl)-1,3-dimethyl-2-oxo-1,2-dihydro-1,6-naphthyridin-5-yl)-5,6,7,8-tetrahydropyrido[3,2-d]pyrimidin-2-yl)picolinate

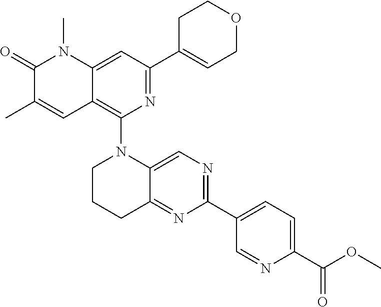

To the solution of methyl 5-(5-(7-chloro-1,3-dimethyl-2-oxo-1,2-dihydro-1,6-naphthyridin-5-yl)-5,6,7,8-tetrahydropyrido[3,2-d]pyrimidin-2-yl)picolinate (50 mg, 105 μmol, 1 eq.) and 2-(3,6-dihydro-2H-pyran-4-yl)-4,4,5,5-tetramethyl-1,3,2-dioxaborolane (44 mg, 210 μmol, 2 eq.) and K₂CO₃ (43.5 mg, 315 μmol, 3 eq.) in 1,4-Dioxane: H₂O=10:1 (3 mL) was added Pd(dppf)Cl₂ (8 mg, 10.5 μmol, 0.1 eq) at 25° C. The mixture was stirred at 100° C. for 2 hours under N₂ atmosphere. The reaction was monitored by LCMS. The resulting solution was diluted with 50 ml of water, then extracted with EtOAc (10 mL×3) and washed with brine (10 mL×3). The residue was purified by flash column chromatography on silica gel (100-200 mesh silica gel, 0-30% EtOAc in PE) to afford methyl 5-(5-(7-(3,6-dihydro-2H-pyran-4-yl)-1,3-dimethyl-2-oxo-1,2-dihydro-1,6-naphthyridin-5-yl)-5,6,7,8-tetrahydropyrido[3,2-d]pyrimidin-2-yl)picolinate (40 mg, 73% yield) as a brown solid.

LC-MS (ESI): mass calcd. for C₂₉H₂₈N₆O₄, 524; m/z found, 525 [M+H+.

### Step B: methyl 5-(5-(1,3-dimethyl-2-oxo-7-(tetrahydro-2H-pyran-4-yl)-1,2-dihydro-1,6-naphthyridin-5-yl)-5,6,7,8-tetrahydropyrido[3,2-d]pyrimidin-2-yl)picolinate

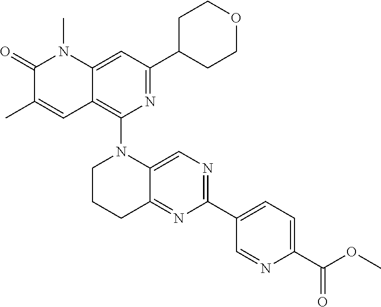

To the solution of methyl 5-(5-(7-(3,6-dihydro-2H-pyran-4-yl)-1,3-dimethyl-2-oxo-1,2-dihydro-1,6-naphthyridin-5-yl)-5,6,7,8-tetrahydropyrido[3,2-d]pyrimidin-2-yl)picolinate (30 mg, 57.2 μmol, 1 eq.) in MeOH (4 mL) was added Pd/C (30 mg, w.t.=10%) at 25° C. The mixture was stirred at 50° C. for 3 hours under H₂ atmosphere. The reaction was monitored by LCMS. The solid was filtered out. The filtrate was concentrated under vacuum to afford methyl 5-(5-(1,3-dimethyl-2-oxo-7-(tetrahydro-2H-pyran-4-yl)-1,2-dihydro-1,6-naphthyridin-5-yl)-5,6,7,8-tetrahydropyrido[3,2-d]pyrimidin-2-yl)picolinate (28 mg, 93% yield) as a yellow solid. LC-MS (ESI): mass calcd. for C₂₉H₃₀N₆O₄, 526; m/z found, 527 [M+H]⁺.

### Intermediate 46. 3-(2-bromo-4-fluorobenzofuran-7-yl)piperidine-2,6-dione

### Step A: ethyl 2-(4-fluorobenzofuran-7-yl)acetate

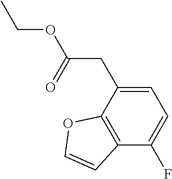 [SMILES: CCOC(=O)Cc1ccc(F)c2ccoc12]

To a flask containing 7-bromo-4-fluorobenzofuran (1 g, 4.65 mmol, 1 eq.), EthylPotassiumMalonate (950 mg, 5.58 mmol, 1.2 eq.), BINAP (174 mg, 279 μmol, 0.06 eq.) and Allylpalladium(II)chloridedimer (34 mg, 93 mmol, 0.02 eq.) in 1,3,5-Trimethylbenzene (5 mL) was added DMAP (56 mg, 465 μmol, 0.1 eq.) at room temperature. The mixture was stirred at 140° C. for 16 hours under N₂ atmosphere. The reaction was monitored by LCMS. The resulting solution was diluted with 50 ml of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-5% EtOAc in PE) to afford ethyl 2-(4-fluorobenzofuran-7-yl)acetate (240 mg, yield 23%) as a yellow solid. LC-MS (ESI): mass calcd. for C₁₂H₁₁FO₃, 222.0; m/z found, 223.1 [M+H]⁺.

### Step B: 3-(4-fluorobenzofuran-7-yl)piperidine-2,6-dione

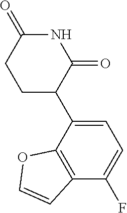 [SMILES: O=C1CCC(c2ccc(F)c3ccoc23)C(=O)N1]

To a solution of ethyl 2-(4-fluorobenzofuran-7-yl)acetate (240 mg, 1.08 mmol, 1 eq.) and acrylamide (307 mg, 4.32 mmol, 4 eq.) in DMF (5 mL) was added t-BuOK (121 mg, 1.08 mmol, 1 eq.) at 25° C. The mixture was stirred at 25° C. for 2 hours under N₂ atmosphere. The reaction was monitored by LCMS. The resulting solution was diluted with 50 ml of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford 3-(4-fluorobenzofuran-7-yl)piperidine-2,6-dione (200 mg, yield 75%) as a yellow solid. LC-MS (ESI): mass calcd. for C₁₃H₁₀FNO₃, 247.0; m/z found, 248.2 [M+H]⁺.

### Step C: 3-(2-bromo-4-fluorobenzofuran-7-yl)piperidine-2,6-dione

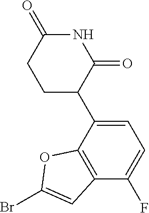 [SMILES: O=C1CCC(c2ccc(F)c3cc(Br)oc23)C(=O)N1]

To a solution of 3-(4-fluorobenzofuran-7-yl)piperidine-2,6-dione (200 mg, 800 μmol, 1 eq.) and NBS (144 mg, 800 μmol, 1 eq.) in MeCN (5 mL) was added 2 drops DMF at 25° C. The mixture was stirred at 80° C. for 16 hours under N₂ atmosphere. The reaction was monitored by LCMS. The resulting solution was diluted with 50 ml of water and extracted with EtOAc. The combined organic layers were washed with brine, dried over anhydrous Na₂SO₄ and concentrated under reduced pressure. The residue was purified by flash column chromatography on silica gel (100-200 mesh silica gel, 0-10% EtOAc in PE) to afford 3-(2-bromo-4-fluorobenzofuran-7-yl)piperidine-2,6-dione (50 mg, yield 19%) as a white solid. LC-MS (ESI): mass calcd. for C₁₃H₉BrFNO₃, 325.0; m/z found, 326.1 [M+H]⁺.

### Intermediate 47. 3-(2-(3-aminoprop-1-yn-1-yl)-1-methyl-1H-benzo[d]imidazol-4-yl)piperidine-2,6-dione

### Step A: 4-(2,6-bis(benzyloxy)pyridin-3-yl)-1-methyl-1H-benzo[d]imidazole

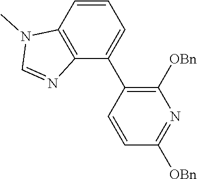 [SMILES: Cn1cnc2c(-c3ccc(OCc4ccccc4)nc3OCc3ccccc3)cccc21]

A mixture of 4-bromo-1-methyl-1H-benzo[d]imidazole (3 g, 14.2 mmol, 1.0 eq.), 2,6-bis(benzyloxy)-3-(4,4,5,5-tetramethyl-1,3,2-dioxaborolan-2-yl)pyridine (7.1 g, 17.1 mmol, 1.2 eq.), Cs₂CO₃ (11.6 g, 35.5 mmol, 2.5 eq.) and Pd(PPh₃)₂Cl₂ (500 mg, 711 μmol, 0.1 eq.) in 1,4-Dioxane (50 mL)/Water (10 mL) was stirred at 90° C. for 16 hours under N₂. The resulting solution was diluted with 20 mL of water and extracted with EtOAc (30 mL×3). The combined organic layers were washed with brine (20 mL), dried over Na₂SO₄ and concentrated under reduced pressure. The residue was purified by flash column chromatography on silica gel (100-200 mesh silica gel, 0-60% EtOAc in PE) to give 4-(2,6-bis(benzyloxy)pyridin-3-yl)-1-methyl-1H-benzo[d]imidazole (4.2 g, yield of 70%) as a yellow solid

LC-MS (ESI): mass calcd. for C₂₇H₂₃N₃O₂, 421.18; m/z found, 422.3 [M+H]⁺.

### Step B: 3-(1-methyl-1H-benzo[d]imidazol-4-yl)piperidine-2,6-dione

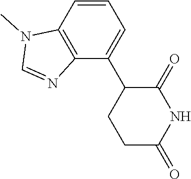 [SMILES: Cn1cnc2c(C3CCC(=O)NC3=O)cccc21]

To a mixture of 4-(2,6-bis(benzyloxy)pyridin-3-yl)-1-methyl-1H-benzo[d]imidazole (2.0 g, 4.74 mmol, 1.0 eq.) in EtOH (50 mL) was added Pd/C (505 mg, w.t--10%,) under H₂. The reaction mixture was stirred at 50° C. using a H₂ balloon for 16 hours to give black solution. The reaction mixture was filtrated and concentrated in vacuum to give 3-(1-methyl-1H-benzo[d]imidazol-4-yl) piperidine-2,6-dione (900 mg, yield of 78%) as white solid.

LC-MS (ESI): mass calcd. for C₁₃H₁₃N₃O₂, 243.10; m/z found, 244.3 [M+H]⁺.

### Step C: 3-(2-bromo-1-methyl-1H-benzo[d]imidazol-4-yl)piperidine-2,6-dione

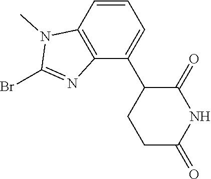 [SMILES: Cn1c(Br)nc2c(C3CCC(=O)NC3=O)cccc21]

To a solution of 3-(1-methyl-1H-benzo[d]imidazol-4-yl) piperidine-2,6-dione (1.0 g, 4.11 mmol, 1.0 eq.) in DCE (20 mL) was added 1-bromopyrrolidine-2,5-dione (878 mg, 4.93 mmol, 1.2 eq.) and stirred at 70° C. for 16 hours. The reaction was a concentrated in vacuum and purified by flash column chromatography on silica gel (100-200 mesh silica gel, 0-100% EtOAc in PE) to give 3-(2-bromo-1-methyl-1H-benzo[d]imidazol-4-yl) piperidine-2,6-dione (700 mg, 53% yield) as a yellow solid. LC-MS (ESI): mass calcd. for C₁₃H₁₂BrN₃O₂, 321.01 323.01; m/z found, 322.3 324.3[M+H]⁺.

### Step D: tert-butyl (3-(4-(2,6-dioxopiperidin-3-yl)-1-methyl-1H-benzo[d]imidazol-2-yl)prop-2-yn-1-yl)carbamate

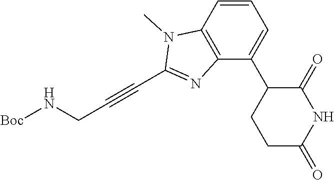 [SMILES: Cn1c(C#CCNC(=O)OC(C)(C)C)nc2c(C3CCC(=O)NC3=O)cccc21]

To a solution of 3-(2-bromo-1-methyl-1H-benzo[d]imidazol-4-yl)piperidine-2,6-dione (200 mg, 621 μmol, 1.0 eq.) in DMF (7 mL) was added CuI (24 mg, 124 μmol, 0.2 Eq.), TEA (628 mg, 6.21 mmol, 10 eq.), Cy³P (35 mg, 124 μmol, 0.2 eq.), Pd(PPh₃)C₁₂ (44 mg, 62.1 μmol, 0.1 eq.) and tert-butyl prop-2-yn-1-ylcarbamate (385 mg, 2.48 mmol, 4.0 eq.) in DMF (2 mL) and stirred at 80° C. for 48 hours. The reaction mixture was extracted with EtOAc (20 mL*3) and water (20 mL). The resulting solution was concentrated in vacuum to give a residue. The residue was purified by flash column chromatography on silica gel (100-200 mesh silica gel, 0-100% EtOAc in PE) to give tert-butyl (3-(4-(2,6-dioxopiperidin-3-yl)-1-methyl-1H-benzo[d]imidazol-2-yl)prop-2-yn-1-yl)carbamate (40.0 mg, 6% yield) as a yellow solid. LC-MS (ESI): mass calcd. for C₂₁H₂₄N₄O₄, 396.18; m/z found, 397.3 [M+H]⁺.

### Step E: 3-(2-(3-aminoprop-1-yn-1-yl)-1-methyl-1H-benzo[d]imidazol-4-yl)piperidine-2,6-dione

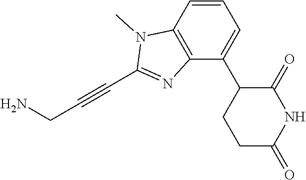 [SMILES: Cn1c(C#CCN)nc2c(C3CCC(=O)NC3=O)cccc21]

A mixture of tert-butyl (3-(4-(2,6-dioxopiperidin-3-yl)-1-methyl-1H-benzo[d]imidazol-2-yl) prop-2-yn-1-yl) carbamate (30 mg, 50.4 μmol, 1.0 eq.) in DCM (5 mL) was added TFA (1 mL) and stirred at 30° C. for 1 hour. The reaction was concentrated to give 3-(2-(3-aminoprop-1-yn-1-yl)-1-methyl-1H-benzo[d]imidazol-4-yl) piperidine-2,6-dione (15.0 mg, yield of 90%,) and direct use for the next step. LC-MS (ESI): mass calcd. for C₁₆H₁₆N₄O₂, 296.13; m/z found, 279.3 [M+H-17]+.

### Intermediate 48. 5-bromo-1,3-dimethyl-7-(tetrahydro-2H-pyran-4-yl)quinoxalin-2(1H)-one

### Step A: 2-bromo-4-iodo-6-nitroaniline

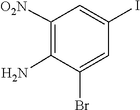 [SMILES: Nc1c(Br)cc(I)cc1[N+](=O)[O-]]

To a solution of 2-bromo-6-nitroaniline (5 g, 23. mmol, 1.0 eq.) in AcOH (50 mL) was added NIS (6.2 g, 27.6 mmol, 1.2 eq.) under N₂. The solution was stirred at 50° C. for 16 hours to give yellow solution. TLC showed the reaction was completed. The solution was quenched water, filtered and concentrated to afford 2-bromo-4-iodo-6-nitroaniline (6.5 g, yield 78%) as a yellow solid. The product was no Ms signal. ¹H NMR (400 MHz, DMSO-d₆) δ 8.28 (d, J=2.0 Hz, 1H), 8.12 (t, J=1.6 Hz, 1H), 7.27 (s, 2H).

### Step B: 3-bromo-5-iodobenzene-1,2-diamine

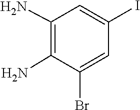

To a solution of 2-bromo-4-iodo-6-nitroaniline (6.5 g, 19 mmol, 1.0 eq.) in EtOH (50 mL) and Water (50 mL) was added NH₄Cl (10 g, 190 mmol, 10.0 eq.) and iron (11 g, 190 mmol, 10.0 eq.) under N₂. The solution was stirred at 80° C. for 3 hours to give black solution. LCMS showed the reaction was completed. The resulting solution was diluted with 200 mL of water and extracted with EtOAc (3×200 mL). The combined organic layers were washed with brine (3×200 mL), dried over Na₂SO₄, and concentrated under reduced pressure. The residue was purified by flash chromatography (100-200 mesh silica gel, 0% ˜50% EtOAc in PE) to afford 3-bromo-5-iodobenzene-1,2-diamine (5.6 g, yield 90%) as a yellow solid. LC-MS (ESI): mass calcd. for C₆H₆BrIN₂, 311.9/313.9; m/z found, 312.9/314.9 [M+H]⁺.

### Step C: 5-bromo-7-iodo-3-methylquinoxalin-2(1H)-one

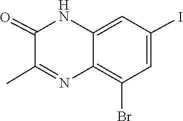

To a solution of 3-bromo-5-iodobenzene-1,2-diamine (5 g, 16 mmol, 1.0 eq.) in AcOH (5 mL) and Water (50 mL) was added ethyl 2-oxopropanoate (3.7 g, 32 mmol, 2.0 eq.). The solution was stirred at 25° C. for 16 hours to give yellow solution. LCMS showed the reaction was completed. The solution was concentrated to afford 5-bromo-7-iodo-3-methylquinoxalin-2(1H)-one (2.3 g, yield 39%) and 8-bromo-6-iodo-3-methylquinoxalin-2(1H)-one (2.3 g, yield 39%) as a yellow solid. LC-MS (ESI): mass calcd. for C₉H₆BrIN₂O, 364.1/366.1; m/z found, 365.1/367.1 [M+H]⁺.

### Step D: 5-bromo-7-iodo-1,3-dimethylquinoxalin-2(1H)-one

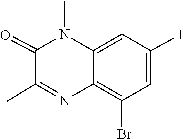

To a solution of 5-bromo-7-iodo-3-methylquinoxalin-2(1H)-one (4.5 g, 12.3 mmol, 1.0 eq.) in DMF (50 mL) was added Cs₂CO₃ (6 g, 18.5 mmol, 1.5 eq.) and Mel (1.54 mL, 24.7 mmol, 2.0 eq.). The solution was stirred at 25° C. for 2 hours to give yellow solution. LCMS showed the reaction was completed. The resulting solution was diluted with 100 mL of water and extracted with EtOAc (3×100 mL). The combined organic layers were washed with brine (3×100 mL), dried over Na₂SO₄, and concentrated under reduced pressure. The residue was purified by flash chromatography (100-200 mesh silica gel, 0% ˜50% EtOAC in PE) to afford 5-bromo-7-iodo-1,3-dimethylquinoxalin-2(1H)-one (1 g, yield 20%) as a yellow solid. LC-MS (ESI): mass calcd. for C₁₀H₈BrIN₂O, 378.2/380.2; m/z found, 379.2/381.2 [M+H]⁺.

### Step E: 5-bromo-1,3-dimethyl-7-(tetrahydro-2H-pyran-4-yl)quinoxalin-2(1H)-one

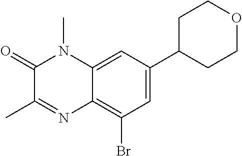 [SMILES: Cc1nc2c(Br)cc(C3CCOCC3)cc2n(C)c1=O]

To a solution of 5-bromo-7-iodo-1,3-dimethylquinoxalin-2(1H)-one (400 mg, 1.1 mmol, 1.0 eq.) in DMA (15 mL) was added TMSCl (401 μL, 3.2 mmol, 3.0 eq.), 1,3-dioxoisoindolin-2-yl tetrahydro-2H-pyran-4-carboxylate (436 mg, 1.6 mmol, 1.5 eq.), Zinc (552 mg, 8.4 mmol, 8.0 eq.) and (biby)₂Ni2(u-C₁)₂C₁₂(H₂O₂) (68 mg, 106 μmol, 0.1 eq.) at 0° C. The solution was stirred at 0° C. for 2 hours to give yellow solution. LCMS showed the reaction was completed. The resulting solution was diluted with 50 mL of water and extracted with EtOAc (3×50 mL). The combined organic layers were washed with brine (3×50 mL), dried over Na₂SO₄, and concentrated under reduced pressure. The residue was purified by flash chromatography (100-200 mesh silica gel, 0% ˜50% EtOAC in PE) to afford 5-bromo-1,3-dimethyl-7-(tetrahydro-2H-pyran-4-yl)quinoxalin-2(1H)-one (45.0 mg, yield 12%) as a yellow solid. LC-MS (ESI): mass calcd. for C₁₅H₁₇BrN₂O₂, 336.0/338.0; m/z found, 337.0/339.0 [M+H]⁺.

### Intermediate 49. 3-(2-bromobenzo[b]thiophen-4-yl)piperidine-2,6-dione

### Step A: 2-iodo-4-methylbenzo[b]thiophene

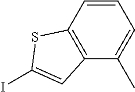

To a mixture of 4-methylbenzo[b]thiophene (700 mg, 4.7 mmol, 1.0 eq.) in THF (10 mL) was added n-BuLi (2.2 mL, 5.7 mmol, 2.5 M in hexane) at −78° C. The mixture was stirred at −78° C. for 30 minutes. Then added 12 (1.4 g, 5.7 mmol, 1.2 eq.) and stirred at −78° C. for 1 hour under N₂ atmosphere. The mixture was quenched NH₄Cl solution and extracted with EtOAc (30 mL×3). The combined organic layers were washed with brine, dried over Na₂SO₄ and concentrated under reduced pressure. The residue was purified by flash column chromatography on silica gel (100-200 mesh silica gel, 0-30% EtOAc in PE) to afford 2-iodo-4-methylbenzo[b]thiophene (1.0 g, 77% yield) as a yellow oil. The product was no Ms signal.

¹H NMR (400 MHz, DMSO-d₆) δ 7.81 (d, J=2.8 Hz, 1H), 7.76 (d, J=7.0 Hz, 1H), 7.24 (td, J=7.9, 3.4 Hz, 1H), 7.17-7.13 (m, 1H), 2.55 (d, J=2.4 Hz, 3H).

### Step B: 2-bromo-4-(bromomethyl)benzo[b]thiophene

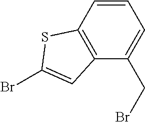 [SMILES: BrCc1cccc2sc(Br)cc12]

The mixture of the 2-do-4-methylbenzo[b]thiophene (700 mg, 2.5 mmol, 1.0 eq.) and NBS (682 mg, 3.8 mmol, 1.5 eq.) in CCl4 (8 mL) was added BPO (124 mg, 511 μmol, 0.2 eq.) at room temperature. The mixture was stirred at 85° C. for 3 hours under N₂ atmosphere. The resulting solution was diluted with 40 mL of H₂O and extracted with DCM (30 mL×3). The combined organic layers were washed with brine, dried over Na₂SO₄ and concentrated under reduced pressure. The residue was purified by flash column chromatography on silica gel (100-200 mesh silica gel, 0-30% EtOAc in PE) to afford 4-(bromomethyl)-2-iodobenzo[b]thiophene (400 mg, 39% yield) as white solid. The product was no Ms signal.

¹H NMR (400 MHz, DMSO-d₆) δ 8.00 (d, J=8.0 Hz, 1H), 7.92 (s, 1H), 7.54 (d, J=7.3 Hz, 1H), 7.42 (t, J=7.7 Hz, 1H), 5.12 (s, 2H).

### Step C: 2-(2-bromobenzo[b]thiophen-4-yl)acetonitrile

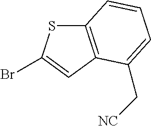 [SMILES: N#CCc1cccc2sc(Br)cc12]

To a solution of TMSCN (96 μL, 735 μmol, 1.5 eq.) in MeCN (5 mL) was added TBAF (882 μL, 882 μmol,1.0 M in THF,) and 2-bromo-4-(bromomethyl)benzo[b]thiophene (150 mg, 490 μmol, 1.5 eq.) at 0° C. The mixture was stirred at room temperature for 2 hours under N₂ atmosphere. The resulting solution was diluted with 30 mL of H₂O and extracted with EtOAc (20 mL×3). The combined organic layers were washed with brine, dried over Na₂SO₄ and concentrated under reduced pressure. The residue was purified by flash column chromatography on silica gel (100-200 mesh silica gel, 0-30% EtOAc in PE) to afford 4-(bromomethyl)-2-iodobenzo[b]thiophene (100 mg, 79% yield) as a yellow oil. The product was no Ms signal. ¹H NMR (400 MHz, DMSO-d₆) δ 8.02-7.89 (m, 1H), 7.80 (s, 1H), 7.41 (d, J=6.6 Hz, 2H), 4.35 (s, 2H).

### Step D: methyl 2-(2-bromobenzo[b]thiophen-4-yl)acetate

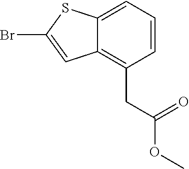

To a solution of 2-(2-bromobenzo[b]thiophen-4-yl)acetonitrile (70 mg, 278 μmol, 1.0 eq.) in MeOH (6 mL) was added SOCl2 (2.0 mL, 278 μmol) slowly. The reaction was stirred at 85° C. for 20 minutes. The mixture was concentrated and diluted with NaHCO₃ solution, extracted with EtOAc (30 mL×3) and the layers were separated. The organic layers were washed with brine (40 mL), dried over Na₂SO₄ filtered and concentrated. The residue was purified by flash column chromatography on silica gel (100-200 mesh silica gel, 0-30% EtOAc in PE) to afford methyl 2-(2-bromobenzo[b]thiophen-4-yl)acetate (55 mg, 68% yield) as a yellow oil. The product was no MS signal. ¹H NMR (400 MHz, DMSO-d₆) δ 7.84 (t, J=14.6 Hz, 1H), 7.68 (d, J=2.3 Hz, 1H), 7.33 (dd, J=7.9, 2.9 Hz, 1H), 7.26 (d, J=7.3 Hz, 1H), 4.02 (d, J=2.2 Hz, 2H), 3.68 (m, 3H).

### Step E: 3-(2-bromobenzo[b]thiophen-4-yl)piperidine-2,6-dione

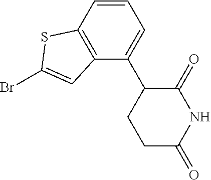 [SMILES: O=C1CCC(c2cccc3sc(Br)cc23)C(=O)N1]

To a solution of methyl 2-(2-bromobenzo[b]thiophen-4-yl)acetate (50 mg, 175 μmol, 1.0 eq.) and acrylamide (37 mg, 525 μmol, 3.0 eq.) in DMF (4 mL) was added t-BuOK (23.6 mg, 210 μmol, 1.2 eq.) at 0° C. under N₂. The reaction was stirred at room temperature for 1 hour. The mixture was diluted with 2 N HCl solution, extracted with EtOAc (20 mL×3) and the layers were separated. The organic layer was washed with brine (20 mL), dried over Na₂SO₄, filtered and concentrated. The residue was purified by flash column chromatography on silica gel (100-200 mesh silica gel, 0-30% EtOAc in PE) to afford 3-(2-bromobenzo[b]thiophen-4-yl)piperidine-2,6-dione (30 mg, 48% yield) as a white solid. LC-MS (ESI): mass calcd. for C₁₃H₁₀BrNO₂S, 322.96; m/z found, 323.96 [M+H]⁺.

### Intermediate 50. 5-bromo-1,4-dimethyl-7-(tetrahydro-2H-pyran-4-yl)quinolin-2(1H)-one

### Step A: 4-(3-bromo-5-fluorophenyl)tetrahydro-2H-pyran

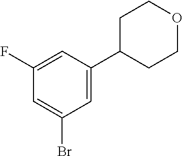 [SMILES: Fc1cc(Br)cc(C2CCOCC2)c1]

To a mixture of 1-bromo-3-fluoro-5-iodobenzene (20 g, 66.5 mmol, 1 eq.) in DMA (300 mL) was added Chlorotrimethylsilane (25.2 mL, 199 mmol, 3 eq.) and 1,3-dioxoisoindolin-2-yl tetrahydro-2H-pyran-4-carboxylate (22 g, 79.8 mmol, 1.2 eq.) and zinc (34.8 g, 532 mmol, 8 eq.) and (biby)₂Ni₂(u-Cl)₂Cl₂(H₂O₂) (4.3 g, 6.7 mmol, 0.1 eq.) under N₂. The mixture was stirred at 0° C. for 1 hour. The resulting solution was diluted with 500 mL of H₂O and extracted with EtOAc (200 mL×3). The combined organic layers were washed with brine, dried over Na₂SO₄, and concentrated under reduced pressure. The residue was purified by flash column chromatography on silica gel (100-200 mesh silica gel, 0-30% EtOAc in PE) to afford 4-(3-bromo-5-fluorophenyl)tetrahydro-2H-pyran (8.0 g, 46% yield) as a yellow oil. No Ms signal.

¹H NMR (400 MHz, DMSO-d₆) δ 7.12-7.06 (m, 2H), 6.96-6.88 (m, 1H), 3.71-3.66 (m, 2H), 3.19-3.13 (m, 2H), 2.60-2.53 (m, 1H), 1.46-1.40 (m, 4H).

### Step B: 2-bromo-6-fluoro-4-(tetrahydro-2H-pyran-4-yl)benzaldehyde

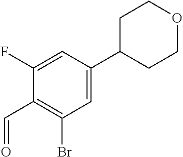 [SMILES: O=Cc1c(F)cc(C2CCOCC2)cc1Br]

To a mixture of 4-(3-bromo-5-fluorophenyl)tetrahydro-2H-pyran (8 g, 30.9 mmol, 1 eq.) in THF (80 mL) was added LDA (30 mL, 34 mmol, 1.1 eq., 1 M in THF) under N₂. The mixture was stirred at −78° C. for 30 mins, then added DMF (2.9 mL, 37 mmol, 1.9 eq.) was stirred at −78° C. for 30 mins. The resulting solution was diluted with 200 mL of H₂O and extracted with EtOAc (100 mL×3). The combined organic layers were washed with brine, dried over Na₂SO₄, and concentrated under reduced pressure. The residue was purified by flash column chromatography on silica gel (100-200 mesh silica gel, 0-30% EtOAc in PE) to afford 2-bromo-6-fluoro-4-(tetrahydro-2H-pyran-4-yl)benzaldehyde (8 g, 86% yield) as a yellow solid.

LC-MS (ESI): mass calcd. for C₁₂H₁₂BrFO₂, 287; m/z found, 287/289 [M+H]⁺.

### Step C: 1-(2-bromo-6-fluoro-4-(tetrahydro-2H-pyran-4-yl)phenyl)ethan-1-ol

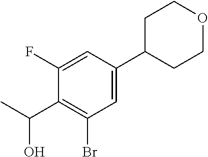 [SMILES: CC(O)c1c(F)cc(C2CCOCC2)cc1Br]

To a solution of 2-bromo-6-fluoro-4-(tetrahydro-2H-pyran-4-yl)benzaldehyde (1 g, 3.5 mmol, 1 eq.) in THF (10 mL) was added Magnesium bromomethyl (1.2 mL, 10.4 mmol, 3 eq.). The reaction mixture was stirred at −65° C. for 2 hours. The resulting solution was diluted with 50 mL of H₂O and extracted with EtOAc (10 mL×3). The combined organic layers were washed with brine, dried over Na₂SO₄, and concentrated under reduced pressure. The residue was purified by flash column chromatography on silica gel (100-200 mesh silica gel, 0-30% EtOAc in PE) to afford 1-(2-bromo-6-fluoro-4-(tetrahydro-2H-pyran-4-yl)phenyl)ethan-1-ol (900 mg, 85.2% yield) as a yellow solid. LC-MS (ESI): mass calcd. for C₁₃H₁₆BrFO₂, 302; m/z found, 285/287 [M+H−18]+.

### Step D: 1-(2-bromo-6-fluoro-4-(tetrahydro-2H-pyran-4-yl)phenyl)ethan-1-one

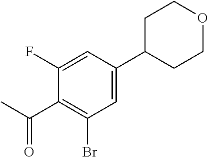 [SMILES: CC(=O)c1c(F)cc(C2CCOCC2)cc1Br]

To a solution of 1-(2-bromo-6-fluoro-4-(tetrahydro-2H-pyran-4-yl)phenyl)ethan-1-ol (900 mg, 3 mmol, 1 eq.) in DCM (15 mL) was added DMP (1.9 g, 4.5 mmol, 1.2 eq.). The reaction mixture was stirred at 0° C. for 4 hours. The resulting solution was diluted with 50 mL of H₂O and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-30% EtOAc in PE) to afford 1-(2-bromo-6-fluoro-4-(tetrahydro-2H-pyran-4-yl)phenyl)ethan-1-one (800 mg, 90% yield) as a yellow solid. LC-MS (ESI): mass calcd. for C₁₃H₁₄BrFO₂, 301; m/z found, 301/303 [M+H]⁺.

### Step E: 1-(2-bromo-6-(methylamino)-4-(tetrahydro-2H-pyran-4-yl)phenyl)ethan-1-one

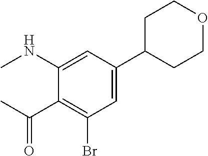 [SMILES: CNc1cc(C2CCOCC2)cc(Br)c1C(C)=O]

To a stirred solution of 1-(2-bromo-6-fluoro-4-(tetrahydro-2H-pyran-4-yl)phenyl)ethan-1-one (800 mg, 2.7 mmol, 1 eq.) and MeNH₂ (3 mL, 3.5 mmol, 1.3 eq. 1.5 mmol in EtOH) and K₂CO₃ (550 mg, 4 mmol, 1.5 eq.) in DMF (10 mL) was stirred at 90° C. for 16 hours under N₂. The resulting solution was diluted with 50 mL of water and extracted with EtOAc (50 mL×3). The combined organic layers were washed with brine (50 mL), dried over Na₂SO₄ and concentrated under reduced pressure to give 1-(2-bromo-6-(methylamino)-4-(tetrahydro-2H-pyran-4-yl)phenyl)ethan-1-one (300 mg, 36% yield) as a yellow solid. LC-MS (ESI): mass calcd. for C₁₄H₁₈BrNO₂, 312; m/z found, 312/314 [M+H]+

### Step F: 5-bromo-1,4-dimethyl-7-(tetrahydro-2H-pyran-4-yl)quinolin-2(1H)-one

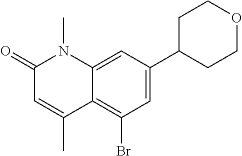 [SMILES: Cc1cc(=O)n(C)c2cc(C3CCOCC3)cc(Br)c12]

To a stirred solution of 1-(2-bromo-6-(methylamino)-4-(tetrahydro-2H-pyran-4-yl)phenyl)ethan-1-one (130 mg, 416 μmol, 1 eq.) and ethyl 2-(diethoxyphosphoryl)acetate (187 mg, 833 μmol, 2 eq.) and K₂CO₃ (172 mg, 1.3 mmol, 3 eq.) in DMF (20 mL) was stirred at 100° C. for 16 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-80% EtOAc in PE) to give 5-bromo-1,4-dimethyl-7-(tetrahydro-2H-pyran-4-yl)quinolin-2(1H)-one (30.0 mg, 21% yield) as a yellow solid.

LC-MS (ESI): mass calcd. for C₁₆H₁₈BrNO₂, 336; m/z found, 336/338 [M+H]⁺.

### Intermediate 51. Methyl 5-(5-((tert-butoxycarbonyl)(methyl)amino)pyrimidin-2-yl)-3-methylpyrazine-2-carboxylate

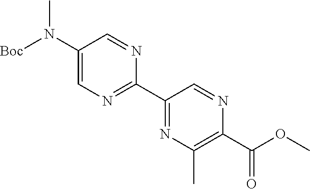 [SMILES: COC(=O)c1ncc(-c2ncc(N(C)C(=O)OC(C)(C)C)cn2)nc1C]

To a solution of tert-butyl (2-bromopyrimidin-5-yl)(methyl)carbamate (300 mg, 1 mmol, 1.0 eq.) in 1,4-Dioxane (3 mL) was added 1,1,1,2,2,2-hexamethyldistannane (510 mg, 1.5 mmol, 1.5 eq.), Pd(PPh₃)₄ (120 mg, 104 μmol, 0.1 eq.) at 25° C. The mixture was then stirred at 125° C. for 2 hours under N₂. The resulting solution was added methyl 5-chloro-3-methylpyrazine-2-carboxylate (194 mg, 1 mmol, 1.0 eq.) at 125° C. under N₂. Then the mixture was stirred at 125° C. for 16 hours under N₂ atmosphere. Then the mixture was concentrated under vacuum. The residue was purified on a silica gel chromatography (100-200 mesh silica gel, 0-60% EtOAc in PE) to give methyl 5-(5-((tert-butoxycarbonyl)(methyl)amino)pyrimidin-2-yl)-3-methylpyrazine-2-carboxylate (60 mg, yield 16%) as a yellow solid. LC-MS (ESI): mass calcd. for C₁₇H₂₁N₅O₄, 359.2; m/z found, 360.2 [M+H]⁺.

### Intermediate 52. 3-(2-iodothieno[3,2-c]pyridin-4-yl)piperidine-2,6-dione

### Step A: 4-chloro-2-iodothieno[3,2-c]pyridine

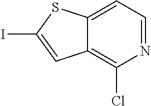 [SMILES: Clc1nccc2sc(I)cc12]

To a solution of 4-chlorothieno[3,2-c]pyridine (5.0 g, 29.5 mmol, 1.0 eq.) in THE (50 mL) was added LDA (17 ml, 35.4 mmol, 1.2 eq., 2 M in THF) at −78° C. The mixture was stirred at −78° C. for 0.5 hours under N₂ atmosphere. Then 12(9.0 g, 35.4 mmol, 1.2 eq.) was added this mixture and stirred at −78° C. for 0.5 hours under N₂ atmosphere. The reaction was monitored by LCMS. The resulting solution was diluted with 60 ml of sat. NH₄Cl and extracted with EtOAc (3*60 mL). The combined organic layers were washed with brine, dried over anhydrous Na₂SO₄ and concentrated under reduced pressure. The residue was purified by flash column chromatography on silica gel (100-200 mesh silica gel, 0-40% EtOAc in PE) to afford 4-chloro-2-iodothieno[3,2-c]pyridine (8.5 g, yield 98%) as a yellow solid.

LC-MS (ESI): mass calcd. for C₇H₃ClINS, 295.1; m/z found, 296.3 [M+H]⁺.

### Step B: tert-butyl 2-cyano-2-(2-iodothieno[3,2-c]pyridin-4-yl)acetate

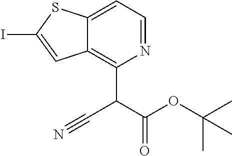 [SMILES: CC(C)(C)OC(=O)C(C#N)c1nccc2sc(I)cc12]

To a solution of 4-chloro-2-iodothieno[3,2-c]pyridine (6.0 g, 20.3 mmol, 1.0 eq.) and K₂CO₃ (8.4 g, 60.9 mmol, 3.0 eq.) in DMSO (60 mL) was added tert-butyl 2-cyanoacetate (2.9 g, 20.3 mmol, 1.0 eq.) at 25° C. The mixture was stirred at 120° C. for 3 hours under N₂ atmosphere. The reaction was monitored by LCMS. The resulting solution was diluted with 50 ml of water and extracted with EtOAc (3*40 mL). The combined organic layers were washed with brine, dried over anhydrous Na₂SO₄ and concentrated under reduced pressure. The residue was purified by flash column chromatography on silica gel (100-200 mesh silica gel, 0-80% EtOAc in PE) to afford tert-butyl 2-cyano-2-(2-iodothieno[3,2-c]pyridin-4-yl)acetate (2.1 g, yield 26%) as a yellow solid. LC-MS (ESI): mass calcd. for C₁₄H₁₃IN₂O₂S, 400.1; m/z found, 401.4 [M+H]⁺.

### Step C: 2-(2-iodothieno[3,2-c]pyridin-4-yl)acetonitrile

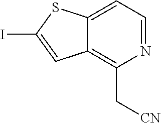 [SMILES: N#CCc1nccc2sc(I)cc12]

To a solution of tert-butyl 2-cyano-2-(2-iodothieno[3,2-c]pyridin-4-yl)acetate (1.0 g, 2.5 mmol, 1.0 eq.) in DCM (6 mL) was added TFA (2 ml) at 25° C. The mixture was stirred at 25° C. for 1 hour. The reaction was monitored by LCMS. The resulting solution was diluted with 30 ml of water and extracted with EtOAc (3*30 mL). The combined organic layers were washed with brine, dried over anhydrous Na₂SO₄ and concentrated under reduced pressure. The residue was purified by flash column chromatography on silica gel (100-200 mesh silica gel, 0-20% EtOAc in PE) to afford 2-(2-iodothieno[3,2-c]pyridin-4-yl)acetonitrile (330 mg, yield 44%) as a white solid. LC-MS (ESI): mass calcd. for C₉H₅IN₂S, 300.1; m/z found, 301.4 [M+H]⁺.

### Step D: ethyl 4-cyano-4-(2-iodothieno[3,2-c]pyridin-4-yl)butanoate

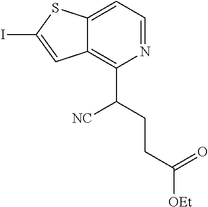 [SMILES: CCOC(=O)CCC(C#N)c1nccc2sc(I)cc12]

To a solution of 2-(2-iodothieno[3,2-c]pyridin-4-yl)acetonitrile (330 mg, 1.1 mmol, 1.0 eq.) and K₂CO₃ (456 mg, 3.3 mmol, 3.0 eq.) in DMF (5 mL) was added ethyl 3-bromopropanoate (199 mg, 1.1 mmol, 1.0 eq.) at 25° C. The mixture was stirred at 50° C. for 0.5 hours under N₂ atmosphere. The reaction was monitored by LCMS. The resulting solution was diluted with 20 ml of water and extracted with EtOAc (3*20 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to give productethyl 4-cyano-4-(2-iodothieno[3,2-c]pyridin-4-yl)butanoate (160 mg, yield 36%) as a yellow oil. LC-MS (ESI): mass calcd. for C₁₄H₁₃IN₂O₂S, 400.1; m/z found, 401.4 [M+H]⁺.

### Step E: 3-(2-iodothieno[3,2-c]pyridin-4-yl)piperidine-2,6-dione

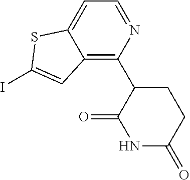 [SMILES: O=C1CCC(c2nccc3sc(I)cc23)C(=O)N1]

To a solution of 4-cyano-4-(2-iodothieno[3,2-c]pyridin-4-yl)butanoate (160 mg, 0.4 mmol, 1.0 eq.) in AcOH (4 mL) was added con. H₂SO₄ (0.4 mL) at 25° C. The mixture was stirred at 100° C. for 1 hour under N₂ atmosphere. The reaction was monitored by LCMS. The resulting solution was diluted with 20 ml of sat. NaHCO₃ and extracted with EtOAc (3*10 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to give 3-(2-iodothieno[3,2-c]pyridin-4-yl)piperidine-2,6-dione (120 mg, yield 81%) as a white solid.

LC-MS (ESI): mass calcd. for C₁₄H₁₃IN₂O₂S, 400.1; m/z found, 401.4 [M+H]⁺.

### Intermediate 53. 7-(4-fluorotetrahydro-2H-pyran-4-yl)-1,3-dimethyl-2-oxo-1,2-dihydroquinolin-5-yl trifluoromethanesulfonate

### Step A: 7-(3,6-dihydro-2H-pyran-4-yl)-5-methoxy-1,3-dimethylquinolin-2(1H)-one

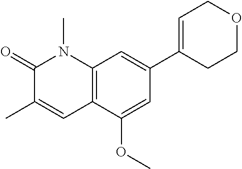 [SMILES: COc1cc(C2=CCOCC2)cc2c1cc(C)c(=O)n2C]

A mixture of 7-bromo-5-methoxy-1,3-dimethylquinolin-2(1H)-one (600 mg, 2.1 mmol, 1.0 eq.), 2-(3,6-dihydro-2H-pyran-4-yl)-4,4,5,5-tetramethyl-1,3,2-dioxa borolane (1.3 g, 6.3 mmol, 3.0 eq.), Pd(dppf)Cl₂ (233 mg, 319 μmol, 0.15 eq.) and Na₂CO₃ (676 mg, 6.3 mmol, 3.0 eq.) in 1,4-Dioxane (10 mL) and H₂O(1 mL) was stirred at 100° C. for 3 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-50% EtOAc and 10% DCM in PE) to give 7-(3,6-dihydro-2H-pyran-4-yl)-5-methoxy-1,3-dimethylquinolin-2(1H)-one (400 mg, 66% yield) as a yellow solid. LC-MS (ESI): mass calcd. for C₁₇H₁₉NO₃, 285.14; m/z found, 286 [M+H]⁺.

### Step B: 7-(4-hydroxytetrahydro-2H-pyran-4-yl)-5-methoxy-1,3-dimethylquinolin-2(1H)-one

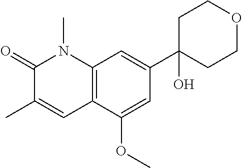 [SMILES: COc1cc(C2(O)CCOCC2)cc2c1cc(C)c(=O)n2C]

To a solution of 7-(3,6-dihydro-2H-pyran-4-yl)-5-methoxy-1,3-dimethylquinolin-2(1H)-one (100 mg, 350 μmol, 1.0 eq.) and Mn(dpm)₃ (63.5 mg, 105 μmol, 0.3 eq.) in IPA (5.00 mL) and DCM (0.60 mL) added PhSiH₃ (49.2 mg, 456 μmol, 1.3 eq.) at 0° C. under 02. The mixture was stirred at 25° C. for 6 hours under 02. The resulting solution was diluted with 50 mL of water and extracted with DCM (50 mL×3). The combined organic layers were washed with brine (50 mL), dried over Na₂SO₄ and concentrated under reduced pressure. The residue was purified by flash column chromatography on silica gel (100-200 mesh silica gel, 0-100% EtOAc in PE) to give 7-(4-hydroxytetrahydro-2H-pyran-4-yl)-5-methoxy-1,3-dimethylquinolin-2(1H)-one (100 mg, 94% yield) as a brown solid. LC-MS (ESI): mass calcd. for C₁₇H₂₁NO₄, 303.15; m/z found, 304 [M+H]⁺.

### Step C: 7-(4-fluorotetrahydro-2H-pyran-4-yl)-5-methoxy-1,3-dimethylquinolin-2(1H)-one

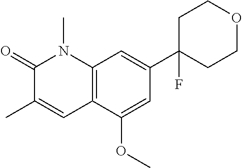 [SMILES: COc1cc(C2(F)CCOCC2)cc2c1cc(C)c(=O)n2C]

A mixture of 7-(4-hydroxytetrahydro-2H-pyran-4-yl)-5-methoxy-1,3-dimethylquinolin-2(1H)-one (80 mg, 264 μmol, 1.0 eq.) and DAST (66 μL, 527 μmol, 2.0 eq.) in DCM (3 mL) was stirred at 0° C. for 3 hours under N₂. The resulting solution was quenched with sat. NaHCO₃ and extracted with DCM (50 mL×3). The combined organic layers were washed with brine (50 mL), dried over Na₂SO₄ and concentrated under reduced pressure to give 7-(4-fluorotetrahydro-2H-pyran-4-yl)-5-methoxy-1,3-dimethylquinolin-2(1H)-one (80 mg, 99% yield) as a white solid. LC-MS (ESI): mass calcd. for C₁₇H₂₀FNO₃, 305.14; m/z found, 306 [M+H]⁺.

### Step D: 7-(4-fluorotetrahydro-2H-pyran-4-yl)-5-hydroxy-1,3-dimethylquinolin-2(1H)-one

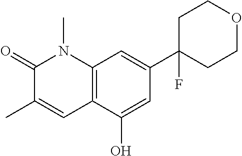 [SMILES: Cc1cc2c(O)cc(C3(F)CCOCC3)cc2n(C)c1=O]

A mixture of 7-(4-fluorotetrahydro-2H-pyran-4-yl)-5-methoxy-1,3-dimethylquinolin-2(1H)-one (60 mg, 196 μmol, 1.0 eq.) and sodium ethanethiolate (165 mg, 2.0 mmol, 10 eq.) in DMF (3 mL) was stirred at 100° C. for 2 hours under N₂. The resulting solution was diluted with 50 mL of water and extracted with EtOAc (10 mL×3). The combined organic layers were washed with brine (50 mL), dried over Na₂SO₄ and concentrated under reduced pressure. The residue was purified by flash column chromatography on silica gel (100-200 mesh silica gel, 0-100% EtOAc in PE) to give 7-(4-fluorotetrahydro-2H-pyran-4-yl)-5-hydroxy-1,3-dimethylquinolin-2(1H)-one (20 mg, 35% yield) as a white solid. LC-MS (ESI): mass calcd. for C₁₆H₁₈FNO₃, 291.13; m/z found, 292 [M+H]⁺.

### Step E: 7-(4-fluorotetrahydro-2H-pyran-4-yl)-1,3-dimethyl-2-oxo-1,2-dihydroquinolin-5-yl trifluoromethanesulfonate

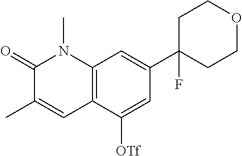 [SMILES: Cc1cc2c(OS(=O)(=O)C(F)(F)F)cc(C3(F)CCOCC3)cc2n(C)c1=O]

To a solution of 7-(4-fluorotetrahydro-2H-pyran-4-yl)-5-hydroxy-1,3-dimethylquinolin-2(1H)-one (30 mg, 103 μmol, 1.0 eq.), Pyridine (33 μL, 412 μmol, 4.0 eq.) in DCM (15 mL) was added trifluoromethanesulfonic anhydride (58 mg, 206 μmol, 2.0 eq.) at 0° C.

The mixture was stirred at 0° C. for 2 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-100% EtOAc in PE) to give 7-(4-fluorotetrahydro-2H-pyran-4-yl)-1,3-dimethyl-2-oxo-1,2-dihydroquinolin-5-yl trifluoromethanesulfonate (20 mg, 46% yield) as a white solid._LC-MS (ESI): mass calcd. for C₁₇H₁₇F₄NO₅S, 423.08; m/z found, 424 [M+H]⁺.

### Intermediate 54. Ethyl 2-(5-bromobenzofuran-3-yl)propanoate

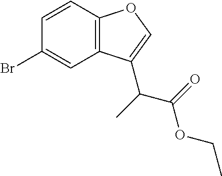 [SMILES: CCOC(=O)C(C)c1coc2ccc(Br)cc12]

To a solution of ethyl 2-(5-bromobenzofuran-3-yl)acetate (200 mg, 706 μmol, 1.0 eq.) in THF (6 mL) was added LiHMDS (1.1 mL, 1.1 mmol, 1.5 eq., 1 M in THF) at −78° C. The reaction was stirred at −78° C. for 30 minutes. Then Mel (120 mg, 848 μmol, 1.2 eq.) was added into the reaction. The mixture was stirred at rt for 2 hours. TLC showed no raw material. Then the mixture was quenched with sat. NH₄Cl. The resulting solution was diluted with 50 ml of water and extracted with EtOAc (50 mL×3). The combined organic layers were washed with brine, dried over Na₂SO₄ and concentrated under reduced pressure. The residue was purified on a silica gel chromatography (100-200 mesh silica gel, 0-60% EtOAc in PE) to give ethyl 2-(5-bromobenzofuran-3-yl) propanoate (130 mg, yield 62%) as a yellow solid. LC-MS (ESI): mass calcd. for C₁₃H₁₃BrO₃, 296.0; m/z found, 297.0/299.0 [M+H]⁺.

### Intermediate 55. methyl 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)pyrazine-2-carboxylate

### Step A: methyl 5-(trimethylstannyl)pyrazine-2-carboxylate

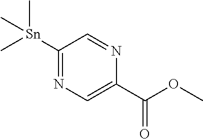

To a solution of methyl 5-chloropyrazine-2-carboxylate (1 g, 5.8 mmol, 1.0 eq.) in Toluene (10 mL) was added Pd(PPh₃)₄ (670 mg, 579 μmol, 0.1 eq.), TBAI (2.4 g, 6.4 mmol, 1.1 eq.) and Sn₂Me6 (1.8 mL, 8.7 mmol, 1.5 eq.) under N₂. The solution was stirred at 110° C. for 3 hours to give black solution. LCMS showed the reaction was completed. The resulting solution was diluted with 100 mL of water and extracted with EtOAc (3×100 mL). The combined organic layers were washed with brine (100 mL), dried over Na₂SO₄, and concentrated under reduced pressure. The residue was purified by flash chromatography (100-200 mesh silica gel, 0%-50% EtOAc in PE) to afford methyl 5-(trimethylstannyl)pyrazine-2-carboxylate (150 mg, yield 8.2%) as a colorless oil. LC-MS (ESI): mass calcd. for C₉H₁₄N₂O₂Sn, 300.9; m/z found, 301.9 [M+H]⁺.

### Step B: methyl 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)pyrazine-2-carboxylate

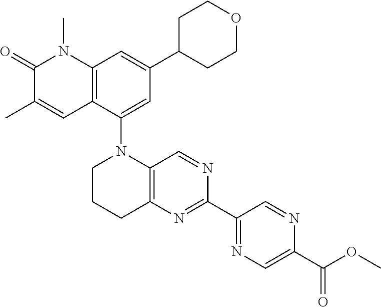

To a solution of methyl 5-(trimethylstannyl)pyrazine-2-carboxylate (130 mg, 432 μmol, 3.0 eq.) in 1,4-Dioxane (5 mL) was added Pd(dtbpf)Cl₂ (19 mg, 28.8 μmol, 0.2 eq.) and 5-(2-chloro-7,8-dihydropyrido[3,2-d]pyrimidin-5(6H)-yl)-1,3-dimethyl-7-(tetrahydro-2H-pyran-4-yl)quinolin-2(1H)-one (61 mg, 144 μmol, 1.0 eq.) under N₂. The solution was stirred at 100° C. for 2 hours to give black solution. LCMS showed the reaction was completed. The resulting solution was diluted with 50 mL of KF solution and extracted with EtOAc (3×50 mL). The combined organic layers were washed with brine (3×50 mL), dried over Na₂SO₄, and concentrated under reduced pressure. The residue was purified by flash chromatography (100-200 mesh silica gel, 0%-100% EtOAc in PE) to afford methyl 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)pyrazine-2-carboxylate (57 mg, yield 71%) as a yellow solid. LC-MS (ESI): mass calcd. for C₂₉H₃₀N₆O₄, 526.6; m/z found, 527.6 [M+H]⁺.

### Intermediate 56. Methyl 5-(8-(7-isopropoxy-1,3-dimethyl-2-oxo-1,2-dihydro-1,6-naphthyridin-5-yl)isoquinolin-3-yl)picolinate

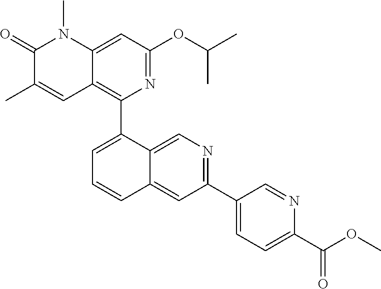

To a solution of methyl 5-(8-(7-chloro-1,3-dimethyl-2-oxo-1,2-dihydro-1,6-naphthyridin-5-yl)isoquinolin-3-yl)picolinate (200 mg, 425 μmol, 1.0 eq.) in i-PrOH (15 mL)was added sodium propan-2-olate (140 mg, 1.7 mmol, 4.0 eq.). The mixture was stirred at 100° C. for 12 hours. The mixture was purified by Prep-HPLC to afford methyl 5-(8-(7-isopropoxy-1,3-dimethyl-2-oxo-1,2-dihydro-1,6-naphthyridin-5-yl)isoquinolin-3-yl)picolinate (20.0 mg, 9% yield) as a white solid. LC-MS (ESI): mass calcd. for C₂₉H₂₆N₄O₄, 494.2; m/z found, 495.2[M+H]⁺.

### Intermediate 57. 6-((3-chloro-1,2,4-triazin-6-yl)(methyl)amino)-1,3-dimethyl-4-(tetrahydro-2H-pyran-4-yl)-1,3-dihydro-2H-benzo[d]imidazol-2-one

### Step A: tert-butyl (1,3-dimethyl-2-oxo-7-(tetrahydro-2H-pyran-4-yl)-2,3-dihydro-1H-benzo[d]imidazol-5-yl)(methyl)carbamate

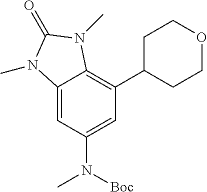 [SMILES: CN(C(=O)OC(C)(C)C)c1cc(C2CCOCC2)c2c(c1)n(C)c(=O)n2C]

To a solution of 1,3-dimethyl-2-oxo-7-(tetrahydro-2H-pyran-4-yl)-2,3-dihydro-1H-benzo[d]imidazol-5-yltrifluoromethanesulfonate (200 mg, 507 μmol, 1.0 eq.) in Tol (3 mL) was added tert-butyl methylcarbamate (86 mg, 659 μmol, 1.3 eq.), Cs₂CO₃ (496 mg, 1.5 mmol, 3.0 eq.), X-Phos (48 mg, 101 μmol, 0.2 eq.) and Pd₂(dba)₃ (93 mg, 101 μmol, 0.2 eq.) at 25° C. The mixture was stirred at 100° C. for 16 hours under N₂. The reaction was monitored by LCMS. The resulting solution was diluted with 30 ml of water, then extracted with EtOAc (3×30 mL) and washed with brine. The combined organic layer was dried over anhydrous sodium sulfate and concentrated under reduced pressure. The residue was purified on a silica gel chromatography (100-200 mesh silica gel, 0-60% EtOAc in PE) to give tert-butyl (1,3-dimethyl-2-oxo-7-(tetrahydro-2H-pyran-4-yl)-2,3-dihydro-1H-benzo[d]imidazol-5-yl)(methyl)carbamate (150 mg, yield 79%) as a yellow solid. LC-MS (ESI): mass calcd. for C₂₀H₂₉N₃O₄, 375.2; m/z found, 376.2 [M+H]⁺.

### Step B: 1,3-dimethyl-6-(methylamino)-4-(tetrahydro-2H-pyran-4-yl)-1,3-dihydro-2H-benzo[d]imidazol-2-one

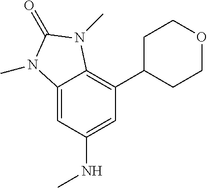 [SMILES: CNc1cc(C2CCOCC2)c2c(c1)n(C)c(=O)n2C]

To a solution of tert-butyl (1,3-dimethyl-2-oxo-7-(tetrahydro-2H-pyran-4-yl)-2,3-dihydro-1H-benzo[d]imidazol-5-yl)(methyl)carbamate (150 mg, 399 μmol, 1.0 eq.) in DCM (3 mL) was added HCl (3 mL, 4M in 1,4-Dioxane) at 25° C. The combined organic layer was concentrated under reduced pressure to give 1,3-dimethyl-6-(methylamino)-4-(tetrahydro-2H-pyran-4-yl)-1,3-dihydro-2H-benzo[d]imidazol-2-one (100 mg, yield 91%) as a yellow solid. LC-MS (ESI): mass calcd. for C₁₅H₂₁N₃O₂, 275.2; m/z found, 276.2 [M+H]⁺.

### Step C: 6-((3-chloro-1,2,4-triazin-6-yl)(methyl)amino)-1,3-dimethyl-4-(tetrahydro-2H-pyran-4-yl)-1,3-dihydro-2H-benzo[d]imidazol-2-one

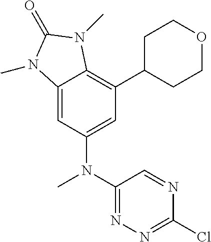 [SMILES: CN(c1cc(C2CCOCC2)c2c(c1)n(C)c(=O)n2C)c1cnc(Cl)nn1]

To a solution of 1,3-dimethyl-6-(methylamino)-4-(tetrahydro-2H-pyran-4-yl)-1,3-dihydro-2H-benzo[d]imidazol-2-one (80 mg, 291 μmol, 1.0 eq.) in Toluene (3 mL) was added 3,6-dichloro-1,2,4-triazine (57 mg, 378 μmol, 1.3 eq.), Cs₂CO₃ (284 mg, 872 μmol, 3.0 eq.), X-Phos (28 mg, 58 μmol, 0.2 eq) and Pd₂(dba)₃ (53 mg, 58 μmol, 0.2 eq) at 25° C. The mixture was then stirred at 60° C. for 2 hours under N₂. LCMS showed the reaction was completed. The mixture was diluted with aq. NH₄Cl and H₂O, extracted with EtOAc (20 mL×3) and the layers were separated. The organic layer was washed with brine, dried over Na₂SO₄, filtered and concentrated. The product was purified by flash chromatography (100-200 mesh silica gel, 0-100% EA in PE) to afford 6-((3-chloro-1,2,4-triazin-6-yl)(methyl)amino)-1,3-dimethyl-4-(tetrahydro-2H-pyran-4-yl)-1,3-dihydro-2H-benzo[d]imidazol-2-one (60 mg, yield 53%) as a yellow solid. LC-MS (ESI): mass calcd. for C₁₈H₂₁ClN₆O₂, 388.1; m/z found, 389.1 [M+H]⁺.

### Intermediate 58. 3-(3-fluoro-2-iodobenzofuran-7-yl)piperidine-2,6-dione

### Step A: ethyl 2-(3-bromo-2-fluorophenyl)-2,2-difluoroacetate

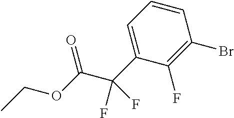 [SMILES: CCOC(=O)C(F)(F)c1cccc(Br)c1F]

To a solution of ethyl 2-bromo-2,2-difluoroacetate (33.7 g, 166 mmol, 2.5 eq.) in DMSO (300 mL) was added Cu (12.7 g, 199 mmol, 3.0 eq.) at 25° C. The mixture was stirred at 25° C. for 1 hour under N₂ atmosphere, after which 1-bromo-2-fluoro-3-iodobenzene (20 g, 66.5 mmol, 1.0 eq.) was added. The resulting reaction mixture was stirred at 70° C. for 3 hours under N₂ atmosphere. The resulting solution was diluted with 300 mL of water and extracted with EtOAc (3×300 mL). The combined organic layers were washed with brine, dried over anhydrous Na₂SO₄ and concentrated under reduced pressure. The residue was purified by flash column chromatography on silica gel (100-200 mesh silica gel, 0-10% EtOAc in PE) to give ethyl 2-(3-bromo-2-fluorophenyl)-2,2-difluoroacetate (15.7 g, 80% yield) as a colorless oil. No Ms signal. ¹HNMR (400 MHz, DMSO-d₆) δ 8.01 (t, J=7.4 Hz, 1H), 7.77-7.71 (m, 1H), 7.38 (t, J=8.0 Hz, 1H), 4.38 (q, J=7.1 Hz, 2H), 1.24 (t, J=7.1 Hz, 3H).

### Step B: 2-(3-bromo-2-fluorophenyl)-2,2-difluoroethan-1-ol

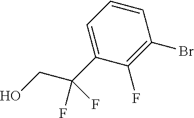 [SMILES: OCC(F)(F)c1cccc(Br)c1F]

To a solution of ethyl 2-(3-bromo-2-fluorophenyl)-2,2-difluoroacetate (15.7 g, 52.8 mmol, 1.0 eq.) in THF (150 mL) was added Lithium borohydride (19.8 mL, 79.3 mmol, 1.5 eq., 4N in THF) at 0° C. The mixture was stirred at 50° C. for 2 hours. The resulting solution was diluted with 100 mL of cold water and extracted with EtOAc (3×100 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to give 2-(3-bromo-2-fluorophenyl)-2,2-difluoroethan-1-ol (12.7 g, 94% yield) as a colorless oil. No MS signal. ¹H NMR (400 MHz, CDCl₃-d₆) 6 7.70 (t, J=7.2 Hz, 1H), 7.55 (t, J=7.2 Hz, 1H), 7.15 (t, J=7.9 Hz, 1H), 4.13 (td, J=13.5, 0.9 Hz, 2H).

### Step C: 7-bromo-3-fluorobenzofuran

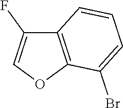 [SMILES: Fc1coc2c(Br)cccc12]

To a solution of 2-(3-bromo-2-fluorophenyl)-2,2-difluoroethan-1-ol (12.7 g, 50 mmol, 1.0 eq.) in THF (150 mL) was added t-BuOK (16.9 g, 150 mmol, 3.0 eq.) and 18-Crown-6 (6.6 g, 25 mmol, 0.5 eq.) at 25° C. The mixture was stirred at 80° C. for 5 hours. The resulting solution was diluted with 100 mL of water and extracted with EtOAc (3×100 mL). The combined organic layers were washed with brine, dried over anhydrous Na₂SO₄ and concentrated under reduced pressure. The residue was purified by flash column chromatography on silica gel (100-200 mesh silica gel, 0-10% EtOAc in PE) to give 7-bromo-3-fluorobenzofuran (6.1 g, 57% yield) as a colorless oil. No Ms signal. ¹H NMR (400 MHz, DMSO-d₆) δ 8.44 (d, J=4.4 Hz, 1H), 7.77-7.66 (m, 2H), 7.36-7.30 (m, 1H).

### Step D: methyl 3-fluorobenzofuran-7-carboxylate

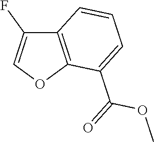

To a solution of 7-bromo-3-fluorobenzofuran (3 g, 14 mmol, 1.0 eq.) in DMF (30 mL): MeOH (30 mL) was added TEA (9.7 mL, 69.8 mmol, 5.0 eq.) and Pd(dppf)Cl₂ (2 g, 2.8 mmol, 0.2 eq.) at 25° C. The mixture was stirred at 80° C. for 5 hours under CO atmosphere. The resulting solution was diluted with 50 mL of water and extracted with EtOAc (3×50 mL). The combined organic layers were washed with brine, dried over anhydrous Na₂SO₄ and concentrated under reduced pressure. The residue was purified by flash column chromatography on silica gel (100-200 mesh silica gel, 0-40% EtOAc in PE) to give methyl 3-fluorobenzofuran-7-carboxylate (2 g, 74% yield) as a white solid. No Ms signal. ¹H NMR (400 MHz, DMSO-d₆) δ 8.43 (d, J=4.3 Hz, 1H), 8.01-7.97 (m, 2H), 7.49 (t, J=7.7 Hz, 1H), 3.93 (s, 3H)

### Step E: (3-fluorobenzofuran-7-yl)methanol

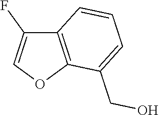 [SMILES: OCc1cccc2c(F)coc12]

To a solutiotiWy1l 3-fluorobenzofuran-7-carboxylate (1.4 g, 7.3 mmol, 1.0 eq.) in THF (15 mL) was added DIBAL-H (10.9 mL, 10.9 mmol, 1.5 eq., 1 N in toluene) at −65° C. The mixture was warmed to 0° C. and stirred for 2 hours under N₂ atmosphere. The reaction was quenched with Potassium sodium tartrate tetrahydrate Solution until no gas detected, then added Celite pad and stirred 10 mins. The mixture was filtered and filtrate was concentrated under reduced pressure. The residue was purified by flash column chromatography on silica gel (100-200 mesh silica gel, 0-20% EtOAc in PE) to give methyl 3-fluorobenzofuran-7-carboxylate (580 mg, 48% yield) as a colorless oil. No MS signal. ¹H NMR (400 MHz, DMSO-d₆) δ 8.26 (d, J=4.4 Hz, 1H), 7.58 (d, J=7.7 Hz, 1H), 7.45 (d, J=7.2 Hz, 1H), 7.34 (t, J=7.6 Hz, 1H), 5.35 (t, J=5.7 Hz, 1H), 4.79 (d, J=5.5 Hz, 2H).

### Step F: tert-butyl((3-fluorobenzofuran-7-yl)methoxy)dimethylsilane

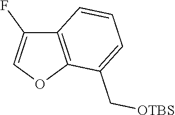 [SMILES: CC(C)(C)[Si](C)(C)OCc1cccc2c(F)coc12]

To a solution of (3-fluorobenzofuran-7-yl)methanol (560 mg, 3.4 mmol, 1.0 eq.) in DCM (10 mL) was added Imidazole (458 mg, 6.7 mmol, 2.0 eq.) and tert-Butyldimethylchlorosilane (762 mg, 5.2 mmol, 1.5 eq.) at 0° C. The mixture was stirred at 0° C. for 1 hour. The resulting solution was diluted with water (50 mL), then extracted with DCM (30 mL×3) and the layers were separated. The combined organic layers were washed with brine, dried over anhydrous Na₂SO₄ and concentrated under reduced pressure. The residue was purified by flash column chromatography on silica gel (100-200 mesh silica gel, 0-5% EtOAc in PE) to afford ert-butyl((3-fluorobenzofuran-7-yl)methoxy)dimethylsilane (900 mg, 95% yield) as a colorless oil. No MS signal.

### Step G: tert-butyl((3-fluoro-2-iodobenzofuran-7-yl)methoxy)dimethylsilane

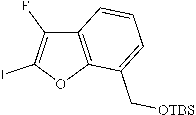 [SMILES: CC(C)(C)[Si](C)(C)OCc1cccc2c(F)c(I)oc12]

To a solution of tert-butyl((3-fluorobenzofuran-7-yl)methoxy)dimethylsilane (900 mg, 3.2 mmol, 1.0 eq.) in THF (10 mL) was added n-BuLi (1.9 mL, 4.8 mmol, 1.5 eq., 2.5M in hexane) at −65° C. under N₂ atmosphere. The reaction was stirred at −65° C. for 30 mins, then added a mixture of 12 (978 mg, 3.7 mmol, 1.2 eq.) in THF. The reaction was stirred at −65° C. for 2 hours.

The mixture was diluted with sat. NH₄Cl solution, extracted with EtOAc (20 mL×3) and the layers were separated. The organic layer was washed with brine, dried over Na₂SO₄, filtered and concentrated. The residue was purified by flash column chromatography on silica gel (100-200 mesh silica gel, 0-15% EtOAc in PE) to afford tert-butyl((3-fluoro-2-iodobenzofuran-7-yl)methoxy)dimethylsilane (1.2 g, 92% yield) as a white solid. No MS signal.

### Step H: (3-fluoro-2-iodobenzofuran-7-yl)methanol

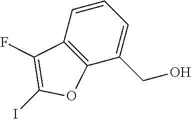 [SMILES: OCc1cccc2c(F)c(I)oc12]

To a solution of tert-butyl((3-fluoro-2-iodobenzofuran-7-yl)methoxy)dimethylsilane (800 mg, 2 mmol, 1.0 eq.) in MeCN (6 mL) was added HCl (2 mL, 4M in dioxane) at 25° C. The reaction was stirred at 25° C. for 2 hours. The mixture was diluted with sat. NaHCO₃ solution, extracted with EtOAc (20 mL×3) and the layers were separated. The organic layer was washed with brine, dried over Na₂SO₄, filtered and concentrated. The residue was purified by flash column chromatography on silica gel (100-200 mesh silica gel, 0-15% EtOAc in PE) to afford (3-fluoro-2-iodobenzofuran-7-yl)methanol (340 mg, 59% yield) as a white solid. No MS signal.

### Step I: (3-fluoro-2-iodobenzofuran-7-yl)methyl methanesulfonate

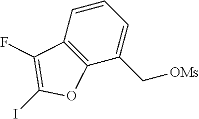 [SMILES: CS(=O)(=O)OCc1cccc2c(F)c(I)oc12]

To a solution of (3-fluoro-2-iodobenzofuran-7-yl)methanol (300 mg, 1.0 mmol, 1.0 eq.) in DCM (10 mL) was added DIEA (268 μL, 1.5 mmol, 1.5 eq.) and MsCl (143 μL, 1.85 mmol, 1.8 eq.) at 0° C. under N₂ atmosphere. The mixture was stirred at 0° C. for 1 hour. The resulting solution was diluted with 20 ml of water and extracted with DCM (3×20 mL). The combined organic layers were washed with brine, dried over anhydrous Na₂SO₄ and concentrated under reduced pressure to afford (3-fluoro-2-iodobenzofuran-7-yl)methyl methanesulfonate (350 mg, 92% yield) as a yellow solid. No MS signal.

### Step J: 2-(3-fluoro-2-iodobenzofuran-7-yl)acetonitrile

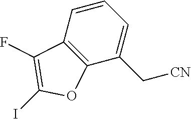 [SMILES: N#CCc1cccc2c(F)c(I)oc12]

To a solution of TMSCN (0.2 mL, 1.4 mmol, 1.5 eq.) in MeCN (5 mL) was added TBAF (1.7 mL, 1.7 mmol, 1.8 eq., 1M in THF) and (3-fluoro-2-iodobenzofuran-7-yl)methyl methanesulfonate (350 mg, 946 μmol, 1.0 eq.) at 0° C. under N₂ atmosphere. The reaction was stirred at 25° C. for 1 hour. The mixture was concentrated under reduced pressure and purified by flash chromatography (100-200 mesh silica gel, 0-15% EtOAc in PE) to afford 2-(3-fluoro-2-iodobenzofuran-7-yl)acetonitrile (280 mg, 98% yield) as a white solid. No MS signal. ¹H NMR (400 MHz, DMSO-d) 6 7.66-7.63 (m, 1H), 7.38 (d, J=5.1 Hz, 2H), 4.31 (s, 2H).

### Step K: ethyl 4-cyano-4-(3-fluoro-2-iodobenzofuran-7-yl)butanoate

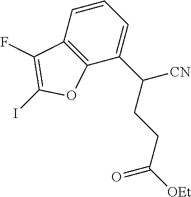 [SMILES: CCOC(=O)CCC(C#N)c1cccc2c(F)c(I)oc12]

To a solution of 2-(3-fluoro-2-iodobenzofuran-7-yl)acetonitrile (280 mg, 930 μmol, 1.0 eq.) in DMF (8 mL) was added ethyl 3-bromopropanoate (168 mg, 930 μmol, 1.0 eq.) and K₂CO₃ (386 mg, 2.79 mmol, 3.0 eq.) at 25° C. The reaction was stirred at 80° C. for 1 hour. The resulting solution was diluted with water (10 mL), then extracted with EtOAc (10 mL×3) and the layers were separated. The combined organic layers were washed with brine, dried over anhydrous Na₂SO₄ and concentrated under reduced pressure. The residue was purified by Prep-TLC (PE:EtOAc=1:1) to afford ethyl 4-cyano-4-(3-fluoro-2-iodobenzofuran-7-yl)butanoate (60 mg, 16% yield) as a colorless oil. LC-MS (ESI): mass calcd. for C₁₅H₁₃FINO₃, 401.0; m/z found, 402.0 [M+H]⁺.

### Step L: ethyl 5-amino-4-(3-fluoro-2-iodobenzofuran-7-yl)-5-oxopentanoate

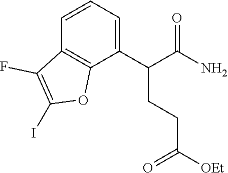 [SMILES: CCOC(=O)CCC(C(N)=O)c1cccc2c(F)c(I)oc12]

To a solution of ethyl 4-cyano-4-(3-fluoro-2-iodobenzofuran-7-yl)butanoate (50 mg, 125 μmol, 1.0 eq.) in Toluene (5 mL) was added Indium trichloride (3 mg, 13 μmol, 0.1 eq.) and Acetaldoxime (22 mg, 374 μmol, 3.0 eq.). The reaction was stirred at 100° C. for 1 hour. The resulting solution was concentrated under reduced pressure and the residue was purified by flash column chromatography on silica gel (100-200 mesh silica gel, 0-50% EtOAc in PE) to afford ethyl 5-amino-4-(3-fluoro-2-iodobenzofuran-7-yl)-5-oxopentanoate (40 mg, 77% yield) as a white solid. LC-MS (ESI): mass calcd. for C₁₅H₁₅FINO₄, 419.0; m/z found, 420.0 [M+H]⁺.

### Step M: 3-(3-fluoro-2-iodobenzofuran-7-yl)piperidine-2,6-dione

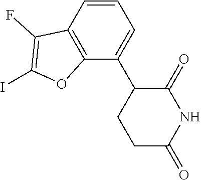 [SMILES: O=C1CCC(c2cccc3c(F)c(I)oc23)C(=O)N1]

To a solution of ethyl 5-amino-4-(3-fluoro-2-iodobenzofuran-7-yl)-5-oxopentanoate (40 mg, 95.4 μmol, 1.0 eq.) in MeCN (5 mL) was added benzyl(trimethyl)ammonium hydroxide (47.9 mg, 286 μmol, 3.0 eq.) at 25° C. The reaction was stirred at 25° C. for 30 mins. The resulting solution was diluted with 20 ml of water, then extracted with EtOAc (10 mL×3) and washed with brine. The combined organic layers were dried over anhydrous Na₂SO₄ and concentrated under reduced pressure. The residue was purified by flash column chromatography on silica gel (100-200 mesh silica gel, 0-50% EtOAc in PE) to afford 3-(3-fluoro-2-iodobenzofuran-7-yl)piperidine-2,6-dione (20 mg, 39% yield, 70% Purity) as a white solid. LC-MS (ESI): mass calcd. for C₁₃H₉FINO₃, 373.0; m/z found, 374.0 [M+H]⁺.

### Intermediate 59. 3-(5-iodofuro[2,3-b]pyridin-3-yl)piperidine-2,6-dione

### Step A: ethyl 5-iodo-3-oxo-2,3-dihydrofuro[2,3-b]pyridine-2-carboxylate

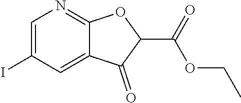 [SMILES: CCOC(=O)C1Oc2ncc(I)cc2C1=O]

To a solution of ethyl 2-hydroxyacetate (3.5 g, 33.6 mmol, 2.0 eq.) in DME (100 mL) was added sodium hydride (1.0 g, 42.0 mmol, 2.5 eq. w.t. =60% in oil) at 0° C. The mixture was 0° C. for 30 mins. Then methyl 2-chloro-5-iodonicotinate (5 g, 16.8 mmol, 1.0 eq.) was added to this mixture. The mixture was stirred at 65° C. for 16 hours under N₂. The resulting solution was diluted with 200 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-30% EtOAc in PE) to give ethyl 5-iodo-3-oxo-2,3-dihydrofuro[2,3-b]pyridine-2-carboxylate (1.5 g, 27% yield) as a white solid. LC-MS (ESI): mass calcd. for C₁₀H₈₁NO₄, 332.95; m/z found, 334 [M+H]⁺.

### Step B: 5-iodofuro[2,3-b]pyridin-3(2H)-one

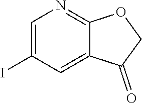 [SMILES: O=C1COc2ncc(I)cc21]

A mixture of ethyl 5-iodo-3-oxo-2,3-dihydrofuro[2,3-b]pyridine-2-carboxylate (2.0 g, 6.0 mmol, 1.0 eq.) in HBr (10 mL) and H₂O(10 mL) was stirred at 80° C. for 16 hours under N₂. The resulting solution was diluted with 50 mL of cool water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-10% EtOAc in PE) to give 5-iodofuro[2,3-b]pyridin-3(2H)-one (1.0 g, 63.8% yield) as a white solid. LC-MS (ESI): mass calcd. for C₇H₄INO₂, 260.93; m/z found, 262 [M+H]⁺.

### Step C: ethyl 2-(5-iodofuro[2,3-b]pyridin-3-yl)acetate

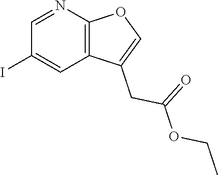 [SMILES: CCOC(=O)Cc1coc2ncc(I)cc12]

A mixture of 5-iodofuro[2,3-b]pyridin-3(2H)-one (1.0 g, 3.8 mmol, 1.0 eq.) and ethyl 2-(triphenyl-15-phosphaneylidene)acetate (1.6 g, 4.6 mmol, 1.2 eq.) in Toluene (15 mL) was stirred at 110° C. for 16 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-10% EtOAc in PE) to give ethyl 2-(5-iodofuro[2,3-b]pyridin-3-yl)acetate (1.0 g, 88% yield) as a yellow solid.

LC-MS (ESI): mass calcd. for C₁₁H₁₀INO₃, 330.97; m/z found, 332 [M+H]⁺.

### Step D: 3-(5-iodofuro[2,3-b]pyridin-3-yl)piperidine-2,6-dione

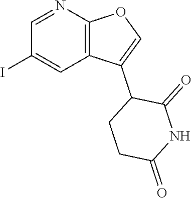 [SMILES: O=C1CCC(c2coc3ncc(I)cc23)C(=O)N1]

A mixture of ethyl 2-(5-iodofuro[2,3-b]pyridin-3-yl)acetate (1.0 g, 3.0 mmol, 1.0 eq.), acrylamide (859 mg, 12.0 mmol, 4.0 eq.) and t-BuOK (338 mg, 3.0 mmol, 1.0 eq.) in DMF (10 mL) was stirred at 25° C. for 3 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-50% EtOAc in PE) to give 3-(5-iodofuro[2,3-b]pyridin-3-yl)piperidine-2,6-dione (300 mg, 27.9% yield) as a yellow solid. LC-MS (ESI): mass calcd. for C₁₂H₉IN₂O₃, 355.97; m/z found, 357 [M+H]⁺.

### Intermediate 60. 3-(2-(3-aminoprop-1-yn-1-yl)-5-fluorobenzofuran-4-yl)piperidine-2,6-dione

### Step A: 4-bromo-5-fluoro-2-iodobenzofuran

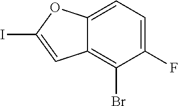 [SMILES: Fc1ccc2oc(I)cc2c1Br]

To a solution of 4-bromo-5-fluorobenzofuran (4 g, 18.6 mmol, 1.0 eq.) in THF (40 mL) was added LDA (28 mL, 28 mmol, 1 M in THF, 1.5 eq.) under N₂, the solution was stirred at −78° C. for 1 hour, then added I² (6.14 g, 24.2 mmol, 1.3 eq.) was stirred at −78° C. for 1 hour to give brown solution. TLC showed the reaction was completed. The solution was quenched water, extracted with EtOAc (200 mL) and the layers were separated. The organic layer was washed with brine (200 mL), dried over Na₂SO₄, filtered and concentrated. The residue was purified by flash chromatography (100-200 mesh silica gel, 0% ˜10% EtOAc in PE) to afford 4-bromo-5-fluoro-2-iodobenzofuran (3 g, yield 45%) as a white oil. No MS signal. ¹H NMR (400 MHz, DMSO-d₆) δ 7.66 (ddt, J=12.8, 9.5, 3.3 Hz, 1H), 7.26 (dddt, J=19.7, 12.6, 6.0, 3.1 Hz, 2H).

### Step B: tert-butyl (3-(4-bromo-5-fluorobenzofuran-2-yl)prop-2-yn-1-yl)carbamate

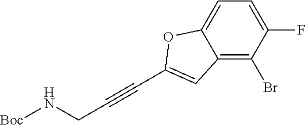 [SMILES: CC(C)(C)OC(=O)NCC#Cc1cc2c(Br)c(F)ccc2o1]

To a solution of 4-bromo-5-fluoro-2-iodobenzofuran (3.7 g, 11 mmol, 1.0 eq.) in DMF (40 mL) was added CuI (207 mg, 1.1 mmol, 0.1 eq.), tert-butyl prop-2-yn-1-ylcarbamate (2.5 g, 16.3 mmol, 1.5 eq.), TEA (4.5 mL, 32.6 mmol, 3.0 eq.) and Pd(PPh₃)₂C₁₂ (762 mg, 1.1 mmol, 0.1 eq.) under N₂. The solution was stirred at 25° C. for 1 hour to give yellow solution. LCMS showed the reaction was completed. The solution was quenched water, extracted with EtOAc (200 mL) and the layers were separated. The organic layer was washed with brine (200 mL), dried over Na₂SO₄, filtered and concentrated. The residue was purified by flash chromatography (100-200 mesh silica gel, 0%-30% EtOAC in PE) to afford tert-butyl (3-(4-bromo-5-fluorobenzofuran-2-yl)prop-2-yn-1-yl)carbamate (3.8 g, yield 90%) as a yellow solid. LC-MS (ESI): mass calcd. for C₁₆H₁₅BrFNO₃, 367.0/369.0; m/z found, 312.0/314.0 [M+H−56]+.

### Step C: tert-butyl (3-(5-fluoro-4-(isoxazol-4-yl)benzofuran-2-yl)prop-2-yn-1-yl)carbamate

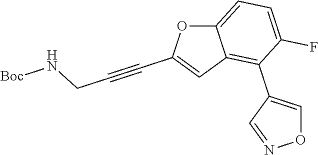 [SMILES: CC(C)(C)OC(=O)NCC#Cc1cc2c(-c3cnoc3)c(F)ccc2o1]

To a solution of tert-butyl (3-(4-bromo-5-fluorobenzofuran-2-yl)prop-2-yn-1-yl)carbamate (900 mg, 2.4 mmol, 1.0 eq.) in DMSO (10 mL) and Water (1 mL) was added Pd(dppf)Cl₂ (179 mg, 244 μmol, 0.1 eq.), 4-(4,4,5,5-tetramethyl-1,3,2-dioxaborolan-2-yl)isoxazole (715 mg, 3.67 mmol, 1.5 eq.) and KF (426 mg, 7.33 mmol, 3.0 eq.) under N₂. The solution was stirred at 90° C. for 3 hours to give black solution. LCMS showed the reaction was completed. The solution was quenched water, extracted with EtOAc (100 mL) and the layers were separated. The organic layer was washed with brine (100 mL), dried over Na₂SO₄, filtered and concentrated. The residue was purified by flash chromatography (100-200 mesh silica gel, 0%-50% EtOAC in PE) to afford tert-butyl (3-(5-fluoro-4-(isoxazol-4-yl)benzofuran-2-yl)prop-2-yn-1-yl)carbamate (550 mg, yield 60%) as a yellow solid. LC-MS (ESI): mass calcd. for C₁₉H₁₇FN₂O₄, 356.3; m/z found, 301.3 [M+H−56]⁺.

### Step D: tert-butyl (3-(4-(cyanomethyl)-5-fluorobenzofuran-2-yl)prop-2-yn-1-yl)carbamate

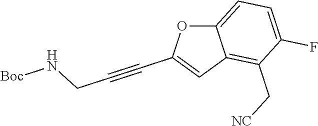 [SMILES: CC(C)(C)OC(=O)NCC#Cc1cc2c(CC#N)c(F)ccc2o1]

To a solution of tert-butyl (3-(5-fluoro-4-(isoxazol-4-yl)benzofuran-2-yl)prop-2-yn-1-yl)carbamate (550 mg, 1.5 mmol, 1.0 eq.) in MeOH (10 mL) and Water (5 mL) was added KF (269 mg, 4.6 mmol, 3.0 eq.) under N₂. The solution was stirred at 90° C. for 1 hour to give yellow solution. LCMS showed the reaction was completed. The solution was quenched water, extracted with EtOAc (50 mL) and the layers were separated. The organic layer was washed with brine (50 mL), dried over Na₂SO₄, filtered and concentrated. The residue was purified by flash chromatography (100-200 mesh silica gel, 0%-50% EtOAc in PE) to afford tert-butyl (3-(4-(cyanomethyl)-5-fluorobenzofuran-2-yl)prop-2-yn-1-yl)carbamate (100 mg, yield 19%) as a yellow solid. LC-MS (ESI): mass calcd. for C₁₈H₁₇FN₂O₃, 328.1; m/z found, 327.1 [M−H]-.

### Step E: methyl 4-(2-(3-((tert-butoxycarbonyl)amino)prop-1-yn-1-yl)-5-fluorobenzofuran-4-yl)-4-cyanobutanoate

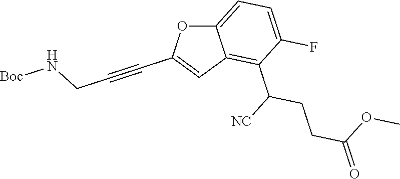 [SMILES: COC(=O)CCC(C#N)c1c(F)ccc2oc(C#CCNC(=O)OC(C)(C)C)cc12]

To a solution of tert-butyl (3-(4-(cyanomethyl)-5-fluorobenzofuran-2-yl)prop-2-yn-1-yl)carbamate (100 mg, 305 μmol, 1.0 eq.) in DMF (5 mL) was added K₂CO₃ (126 mg, 914 μmol, 3.0 eq.) and methyl acrylate (26.2 mg, 305 μmol, 1.0 eq.) under N₂. The solution was stirred at 50° C. for 1 hour to give yellow solution. LCMS showed the reaction was completed. The solution was quenched water, extracted with EtOAc (50 mL) and the layers were separated. The organic layer was washed with brine (50 mL), dried over Na₂SO₄, filtered and concentrated. The residue was purified by flash chromatography (100-200 mesh silica gel, 0%-50% EtOAc in PE) to afford methyl 4-(2-(3-((tert-butoxycarbonyl)amino)prop-1-yn-1-yl)-5-fluorobenzofuran-4-yl)-4-cyanobutanoate (83 mg, yield 62%) as a yellow solid. LC-MS (ESI): mass calcd. for C₂₂H₂₃FN₂O₅, 414.2; m/z found, 359.2 [M+H−56]⁺.

### Step F: methyl 5-amino-4-(2-(3-((tert-butoxycarbonyl)amino)prop-1-yn-1-yl)-5-fluorobenzofuran-4-yl)-5-oxopentanoate

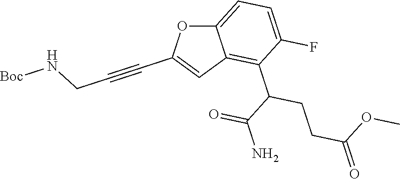 [SMILES: COC(=O)CCC(C(N)=O)c1c(F)ccc2oc(C#CCNC(=O)OC(C)(C)C)cc12]

To a solution of methyl 4-(2-(3-((tert-butoxycarbonyl)amino)prop-1-yn-1-yl)-5-fluorobenzofuran-4-yl)-4-cyanobutanoate (80 mg, 193 μmol, 1.0 eq.) in Toluene (5 mL) was added InCl3 (4.3 mg, 19.3 μmol, 0.1 eq.) and (E)-acetaldehyde oxime (57 mg, 965 μmol, 5.0 eq.) under N₂. The solution was stirred at 110° C. for 2 hours to give yellow solution. LCMS showed the reaction was completed. The solution was quenched with water, extracted with EtOAc (50 mL) and the layers were separated. The organic layer was washed with brine (50 mL), dried over Na₂SO₄, filtered and concentrated. The residue was purified by flash chromatography (100-200 mesh silica gel, 0%-50% EtOAc in PE) to afford methyl 5-amino-4-(2-(3-((tert-butoxycarbonyl)amino)prop-1-yn-1-yl)-5-fluorobenzofuran-4-yl)-5-oxopentanoate (56 mg, yield 64%) as a yellow solid. LC-MS (ESI): mass calcd. for C₂₂H₂₅FN₂O₆, 432.2; m/z found, 377.2 [M+H−56]⁺.

### Step G: tert-butyl (3-(4-(2,6-dioxopiperidin-3-yl)-5-fluorobenzofuran-2-yl)prop-2-yn-1-yl)carbamate

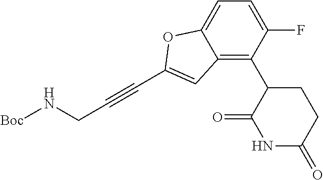 [SMILES: CC(C)(C)OC(=O)NCC#Cc1cc2c(C3CCC(=O)NC3=O)c(F)ccc2o1]

To a solution of methyl 5-amino-4-(2-(3-((tert-butoxycarbonyl)amino)prop-1-yn-1-yl)-5-fluorobenzofuran-4-yl)-5-oxopentanoate (40 mg, 92.5 μmol, 1.0 eq.) in THF (5 mL) was added t-BuOK (21 mg, 185 μmol, 2.0 eq.) under N₂. The solution was stirred at 0° C. for 1 hour to give yellow solution. LCMS showed the reaction was completed. The solution was quenched water, extracted with EtOAc (50 mL) and the layers were separated. The organic layer was washed with brine (50 mL), dried over Na₂SO₄, filtered and concentrated. The residue was purified by flash chromatography (100-200 mesh silica gel, 0%-50% EtOAc in PE) to afford tert-butyl (3-(4-(2,6-dioxopiperidin-3-yl)-5-fluorobenzofuran-2-yl)prop-2-yn-1-yl)carbamate (26 mg, yield 67%) as a yellow solid. LC-MS (ESI): mass calcd. for C₂₁H₂₁FN₂O₅, 400.1; m/z found, 345.1 [M+H−56]+.

### Step H: 3-(2-(3-aminoprop-1-yn-1-yl)-5-fluorobenzofuran-4-yl)piperidine-2,6-dione

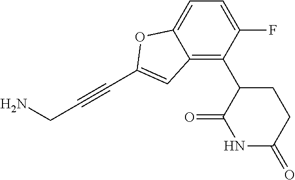 [SMILES: NCC#Cc1cc2c(C3CCC(=O)NC3=O)c(F)ccc2o1]

A solution of tert-butyl (3-(4-(2,6-dioxopiperidin-3-yl)-5-fluorobenzofuran-2-yl)prop-2-yn-1-yl)carbamate (26 mg, 65 μmol, 1.0 eq.) in DCM (5 mL) and TFA (1 mL) was stirred at 25° C. for 30 mins to give yellow solution. LCMS showed the reaction was completed. The solution was concentrated to afford 3-(2-(3-aminoprop-1-yn-1-yl)-5-fluorobenzofuran-4-yl)piperidine-2,6-dione (26 mg, crude) as a yellow oil. LC-MS (ESI): mass calcd. for C₁₆H₁₃FN₂O₃, 300.3; m/z found, 284.3 [M+H-17]+.

### Intermediate 61. Methyl 5-(5-chloroquinazolin-2-yl)picolinate

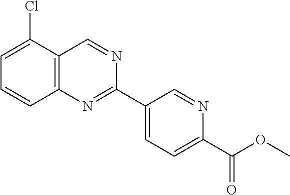 [SMILES: COC(=O)c1ccc(-c2ncc3c(Cl)cccc3n2)cn1]

To a solution of 2,5-dichloroquinazoline (300 mg, 1.5 mmol, 1.0 eq.), Pd(dppf)C₁₂ (110 mg, 150 μmol, 0.1 eq.) and methyl 5-(4,4,5,5-tetramethyl-1,3,2-dioxaborolan-2-yl)picolinate (394.5 mg,, 1.5 mmol, 1.0 eq.) in 1,4-Dioxane (10 mL) and H₂O(1 mL) was added K₂CO₃ (625 mg, 4.5 mmol, 1.0 eq.). The reaction mixture was stirred at 100° C. for 4 hours under N₂. The resulting solution was diluted with 30 mL of H₂O and extracted with EtOAc (40 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give methyl 5-(5-chloroquinazolin-2-yl)picolinate (150 mg, 33% yield) as a yellow solid. LC-MS (ESI): mass calcd. for C₁₅H₁₀ClN₃O₂, 299.05; m/z found, 300.05 [M+H]⁺.

### Intermediate 62. 6-((6-chloro-4-(dimethylamino)pyridin-3-yl)(methyl)amino)-1,3-dimethyl-4-(tetrahydro-2H-pyran-4-yl)-1,3-dihydro-2H-benzo[d]imidazol-2-one

### Step A: 2-chloro-N,N-dimethyl-5-nitropyridin-4-amine

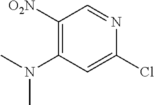

To a solution of 2,4-dichloro-5-nitropyridine (3.0 g, 15.5 mmol, 1 eq.) in THF (30 mL) was added dimethylamine (771 mg, 17.1 mmol, 1.1 eq.) and K₂CO₃ (4.3 g, 31.1 mmol, 2 eq.) at 25° C. The mixture was stirred at 70° C. for 2 hours under N₂ atmosphere. The reaction was monitored by LCMS. The resulting solution was diluted with 100 ml of water and extracted with EtOAc (3* 100 mL). The combined organic layers were washed with brine, dried over anhydrous Na₂SO₄ and concentrated under reduced pressure. The residue was purified by flash column chromatography on silica gel (100-200 mesh silica gel, 0-20% EtOAc in PE) to afford 2-chloro-N,N-dimethyl-5-nitropyridin-4-amine (3.0 g, yield 95%) as a yellow solid. LC-MS (ESI): mass calcd. for C₇H₈ClN₃O₂,201.0; m/z found, 202.0 [M+H]⁺.

### Step B: 6-chloro-N⁴,N⁴-dimethylpyridine-3,4-diamine

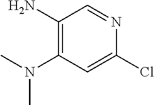

To a solution of 2-chloro-N,N-dimethyl-5-nitropyridin-4-amine (3.0 g, 14.9 mmol, 1 eq.) in EtOH (30 mL) and H₂O(30 mL) was added Ammonium chloride (8.0 g, 149 mmol, 10 eq.) and Fe (8.3 g, 149 mmol, 10 eq.) at 25° C. The mixture was stirred at 80° C. for 16 hours under N₂ atmosphere. The reaction was monitored by LCMS. The resulting solution was diluted with 100 ml of water and extracted with EtOAc (3* 100 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford 6-chloro-N⁴,N⁴-dimethylpyridine-3,4-diamine (1.4 g, yield 55%) as a yellow solid. LC-MS (ESI): mass calcd. for C₇H₁₀ClN₃,171.1; m/z found, 172.1 [M+H]⁺.

### Step C: 6-((6-chloro-4-(dimethylamino)pyridin-3-yl)amino)-1,3-dimethyl-4-(tetrahydro-2H-pyran-4-yl)-1,3-dihydro-2H-benzo[d]imidazol-2-one

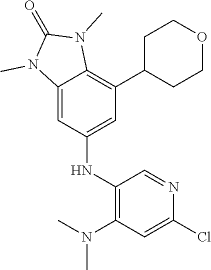 [SMILES: CN(C)c1cc(Cl)ncc1Nc1cc(C2CCOCC2)c2c(c1)n(C)c(=O)n2C]

A solution of 6-chloro-N⁴,N⁴-dimethylpyridine-3,4-diamine (127 mg, 738 μmol, 1.2 eq.), 6-bromo-1,3-dimethyl-4-(tetrahydro-2H-pyran-4-yl)-1,3-dihydro-2H-benzo[d]imidazol-2-one (200 mg, 615 μmol, 1 eq.), Ru-Phos Pd G1 (90 mg, 123 μmol, 0.2 eq.), Ru-Phos (58 mg, 123 μmol, 0.2 eq.) and Cs₂CO₃ (601 mg, 1.84 mmol, 3 eq.) in 1,4-Dioxane (5 mL) was stirred at 100° C. for 2 hours under N₂ atmosphere. The reaction was monitored by LCMS. The resulting solution was diluted with 50 ml of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 6-((6-chloro-4-(dimethylamino)pyridin-3-yl)amino)-1,3-dimethyl-4-(tetrahydro-2H-pyran-4-yl)-1,3-dihydro-2H-benzo[d]imidazol-2-one (40 mg, yield 15%) as a brown solid. LC-MS (ESI): mass calcd. for C₂₁H₂₆ClN₅O₂,415.2; m/z found, 416.2 [M+H]⁺.

### Step D: 6-((6-chloro-4-(dimethylamino)pyridin-3-yl)(methyl)amino)-1,3-dimethyl-4-(tetrahydro-2H-pyran-4-yl)-1,3-dihydro-2H-benzo[d]imidazol-2-one

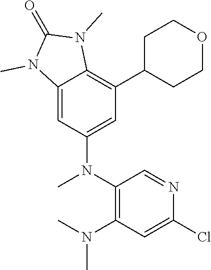 [SMILES: CN(C)c1cc(Cl)ncc1N(C)c1cc(C2CCOCC2)c2c(c1)n(C)c(=O)n2C]

To a solution of 6-((6-chloro-4-(dimethylamino)pyridin-3-yl)amino)-1,3-dimethyl-4-(tetrahydro-2H-pyran-4-yl)-1,3-dihydro-2H-benzo[d]imidazol-2-one (40 mg, 96.2 μmol, 1 eq.) in DMF (2 mL) was added NaH (6 mg, 144 μmol, 1.5 eq., w.t. =60%) at 0° C. and was stirred for 30 mins. The mixture was added Mel (27 mg, 192 μmol, 2 eq.) and was stirred at 25° C. for 1 hour under N₂ atmosphere. The reaction was monitored by LCMS. The resulting solution was diluted with 50 ml of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 6-((6-chloro-4-(dimethylamino)pyridin-3-yl)(methyl)amino)-1,3-dimethyl-4-(tetrahydro-2H-pyran-4-yl)-1,3-dihydro-2H-benzo[d]imidazol-2-one (30 mg, yield 70%) as a brown solid. LC-MS (ESI): mass calcd. for C₂₂H₂₈ClN₅O₂,429.2; m/z found, 430.2 [M+H]⁺.

### Intermediate 63. 7-chloro-4-(7-isopropyl-1,3-dimethyl-2-oxo-2,3-dihydro-1H-benzo[d]imidazol-5-yl)-2-methyl-2,6-naphthyridin-1(2H)-one

### Step A: (E)-N-(tert-butyl)-2-chloro-5-(2-ethoxyvinyl)isonicotinamide

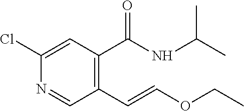 [SMILES: CCO/C=C/c1cnc(Cl)cc1C(=O)NC(C)C]

A solution of 5-bromo-N-(tert-butyl)-2-chloroisonicotinamide (5 g, 17.1 mmol, 1.0 eq.), Pd(dppf)Cl₂ (116 mg, 1.7 mmol, 0.1 eq.), (E)-2-(2-ethoxyvinyl)-4,4,5,5-tetramethyl-1,3,2-dioxaborolane (3.7 g, 18.9 mmol, 1.1 eq.) and K₂CO₃ (4.7 g, 34.3 mmol, 2.0 eq.) in 1,4-Dioxane (80 mL) and Water (8 mL) was stirred at 95° C. for 16 hours under N₂ atmosphere. The resulting solution was diluted with 200 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-30% EtOAc in PE) to give (E)-N-(tert-butyl)-2-chloro-5-(2-ethoxyvinyl)isonicotinamide (2.5 g, 51% yield) as a white solid. LC-MS (ESI): mass calcd. for C₁₄H₁₉ClN₂O₂, 282.11; m/z found, 283 [M+H]⁺.

### Step B: 7-chloro-2,6-naphthyridin-1(2H)-one

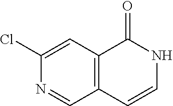 [SMILES: O=c1[nH]ccc2cnc(Cl)cc12]

To a solution of (E)-N-(tert-butyl)-2-chloro-5-(2-ethoxyvinyl) isonicotinamide (2.5 g, 8.8 mmol, 1.0 eq.) in TFA (40 mL) was stirred at 100° C. for 3 days under N₂ atmosphere. After cooled to room temperature, the resulting solution was concentrated under reduced pressure to give 7-chloro-2,6-naphthyridin-1(2H)-one (1.5 g, crude) as a brown solid. LC-MS (ESI): mass calcd. for C₈H₅ClN₂O, 180.01; m/z found, 181 [M+H]⁺.

### Step C: 4-bromo-7-chloro-2,6-naphthyridin-1(2H)-one

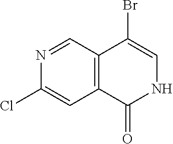 [SMILES: O=c1[nH]cc(Br)c2cnc(Cl)cc12]

To a solution of 7-chloro-2,6-naphthyridin-1(2H)-one (1.5 g, 8.3 mmol, 1.0 eq.) and NBS (1.5 g, 14 mmol, 1.2 eq.) in DCM (10 mL) was stirred at 25° C. for 1 hour under N₂ atmosphere. LCMS showed desired MS. The solid was collected by filtration to give 4-bromo-7-chloro-2,6-naphthyridin-1(2H)-one (1.5 g crude) as a yellow solid. LC-MS (ESI): mass calcd. for C₈H₄BrClN₂O, 257.92; m/z found, 259 [M+H]⁺.

### Step D: 4-bromo-7-chloro-2-methyl-2,6-naphthyridin-1(2H)-one

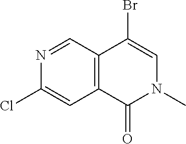

To a stirred solution of 4-bromo-7-chloro-2,6-naphthyridin-1(2H)-one (1.3 g, 5.1 mmol, 1.0 eq.) in DMF (20 mL) was added NaH (492 mg, 20.5 mmol, 4.0 eq. w.t. =60% in oil) at 0° C. After 30 mins of stirring, iodomethane (1.5 g, 10.3 mmol, 2.0 eq.) was dropwise to the solution. The solution was stirred at 25° C. for 30 mins. The resulting solution was diluted with 200 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-50% EtOAc in PE) to give 4-bromo-7-chloro-2-methyl-2,6-naphthyridin-1(2H)-one (700 mg, 44% yield) as a white solid. LC-MS (ESI): mass calcd. for C₉H₆BrClN₂O, 271.94; m/z found, 273 [M+H]⁺.

### Step E: 7-chloro-4-(7-isopropyl-1,3-dimethyl-2-oxo-2,3-dihydro-1H-benzo[d]imidazol-5-yl)-2-methyl-2,6-naphthyridin-1(2H)-one

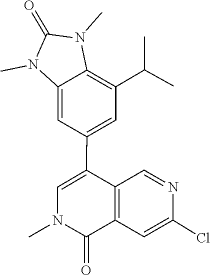 [SMILES: CC(C)c1cc(-c2cn(C)c(=O)c3cc(Cl)ncc23)cc2c1n(C)c(=O)n2C]

A solution of 4-bromo-7-chloro-2-methyl-2,6-naphthyridin-1(2H)-one (120 mg, 438.7 μmol, 1.0 eq.), Pd(dppf)Cl₂ (32 mg, 43.8 μmol, 0.1 eq.), 4-isopropyl-1,3-dimethyl-6-(4,4,5,5-tetramethyl-1,3,2-dioxaborolan-2-yl)-1,3-dihydro-2H-benzo[d]imidazol-2-one (151 mg, 526.5 μmol, 1.2 eq.), and Na₂CO₃ (140 mg, 1.3 mmol, 3.0 eq.) in 1,4-Dioxane (5 mL) and Water (0.5 mL) was stirred at 80° C. for 16 hours under N₂ atmosphere. The resulting solution was diluted with 30 mL of water and extracted with EtOAc (10 mL×3). The combined organic layers were washed with brine (10 mL), dried over Na₂SO₄ and concentrated under reduced pressure. The residue was purified by flash column chromatography on silica gel (100-200 mesh silica gel, 0-10% MeOH in DCM) and then purified by Prep-TLC to give 7-chloro-4-(1,3-dimethyl-2-oxo-2,3-dihydro-1H-benzo[d]imidazol-5-yl)-2-methyl-2,6-naphthyridin-1(2H)-one (18 mg, 12% yield) as a yellow solid. LC-MS (ESI): mass calcd. for C₂₁H₂₁ClN₄O₂, 396.14; m/z found, 397 [M+H]⁺.

### Intermediate 64. 3-(5-bromo-1-methyl-1H-pyrrolo[2,3-b]pyridin-3-yl)piperidine-2,6-dione

### Step A: ethyl 2-(5-bromo-1-methyl-1H-pyrrolo[2,3-b]pyridin-3-yl)-2-oxoacetate

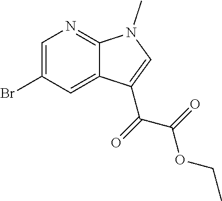 [SMILES: CCOC(=O)C(=O)c1cn(C)c2ncc(Br)cc12]

To a solution of 5-bromo-1-methyl-1H-pyrrolo[2,3-b]pyridine (5 g, 24 mmol, 1.0 eq.) in DCM (50 mL) was added ethyl 2-chloro-2-oxoacetate (19.4 g, 144 mmol, 6.0 eq.) and Aluminum chloride (42.6 g, 364 mmol, 13.5 eq.) at 0° C. The mixture was then stirred at 25° C. for 2 hours under N₂. LCMS showed the reaction was completed. The resulting solution was diluted with 50 mL of water, then extracted with DCM (3×200 mL) and washed with brine. The combined organic layer was dried over anhydrous sodium sulfate and concentrated under vacuum. The residue was purified on a silica gel chromatography (100-200 mesh silica gel, 0-30% EtOAc in PE) to give ethyl 2-(5-bromo-1-methyl-1H-pyrrolo[2,3-b]pyridin-3-yl)-2-oxoacetate (2 g, yield 27%) as a white solid. LC-MS (ESI): mass calcd. for C₁₂H₁₁BrN₂O₃, 310.0; m/z found, 311.0 [M+H]⁺.

### Step B: ethyl 2-(5-bromo-1-methyl-1H-pyrrolo[2,3-b]pyridin-3-yl)acetate

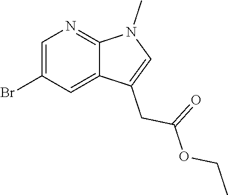 [SMILES: CCOC(=O)Cc1cn(C)c2ncc(Br)cc12]

To a solution of ethyl 2-(5-bromo-1-methyl-1H-pyrrolo[2,3-b]pyridin-3-yl)-2-oxoacetate (2 g, 6.4 mmol, 1.0 eq.) in TFA (20 mL) was added Triethylsilane (4 mL, 25.6 mmol, 4.0 eq.) and at 25° C. The mixture was then stirred at 55° C. for 16 hours under N₂. LCMS showed the reaction was completed. The resulting solution was diluted with 200 mL of water, then extracted with EtOAc (3×200 mL) and washed with brine. The combined organic layer was dried over anhydrous sodium sulfate and concentrated under vacuum. The combined organic layer was dried over anhydrous sodium sulfate and concentrated under vacuum. The residue was purified on a silica gel chromatography (100-200 mesh silica gel, 0-30% EtOAc in PE) to give ethyl 2-(5-bromo-1-methyl-1H-pyrrolo[2,3-b]pyridin-3-yl)-2-oxoacetate (550 mg, yield 29%) as a white solid. LC-MS (ESI): mass calcd. for C₁₂H₁₃BrN₂O₂, 296.0; m/z found, 297.0 [M+H]⁺.

### Step C: 3-(5-bromo-1-methyl-1H-pyrrolo[2,3-b]pyridin-3-yl)piperidine-2,6-dione

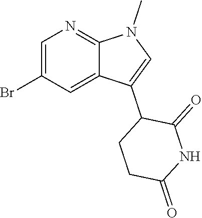 [SMILES: Cn1cc(C2CCC(=O)NC2=O)c2cc(Br)cnc21]

To a solution of ethyl 2-(5-bromo-1-methyl-1H-pyrrolo[2,3-b]pyridin-3-yl)acetate (300 mg, 1 mmol, 1.0 eq.), Ethylene carboxamide (287 mg, 4 mmol, 4.0 eq.), t-BuOK (136 mg, 1.2 mmol, 1.2 eq.) was added DMF (5 mL) at 25° C. The mixture was stirred at 25° C. for 30 minutes. LCMS showed the reaction was completed. The mixture was diluted with aq. NH₄Cl and H₂O, extracted with EtOAc (200 mL×3) and the layers were separated. The organic layer was washed with brine, dried over Na₂SO₄, filtered and concentrated. The product was purified by flash chromatography (100-200 mesh silica gel, 0-80% EtOAc in PE) to afford 3-(5-bromo-1-methyl-1H-pyrrolo[2,3-b]pyridin-3-yl)piperidine-2,6-dione (120 mg, yield 37%) as a yellow solid. LC-MS (ESI): mass calcd. for C₁₃H₁₂BrN₃O₂, 321.0; m/z found, 322.0 [M+H]⁺.

### Intermediate 65. 3-(5-bromofuro[3,2-b]pyridin-3-yl)piperidine-2,6-dione

### Step A: ethyl (E)-4-((6-bromo-2-iodopyridin-3-yl)oxy)but-2-enoate

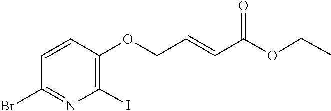 [SMILES: CCOC(=O)/C=C/COc1ccc(Br)nc1I]

To a solution of 6-bromo-2-iodopyridin-3-ol (4 g, 13 mmol, 1.0 eq.), ethyl (E)-4-bromobut-2-enoate (2.6 g, 13 mmol, 1.0 eq.) in Toluene (60 mL) was added silver carbonate (7.4 g, 26 mmol, 2.0 eq.). The mixture was stirred at 80° C. for 3 hours under N₂ atmosphere. LCMS showed the reaction was completed. The resulting solution was diluted with 200 mL of water, then extracted with EtOAc (3×200 mL) and washed with brine. The combined organic layer was dried over anhydrous sodium sulfate and concentrated under vacuum. The residue was purified on a silica gel chromatography (100-200 mesh silica gel, 0-20% EtOAc in PE) to give ethyl (E)-4-((6-bromo-2-iodopyridin-3-yl)oxy)but-2-enoate (4 g, yield 73%) as a white solid.

LC-MS (ESI): mass calcd. for C₁₁H₁₁BrINO₃, 410.9; m/z found, 411.9 [M+H]⁺.

### Step B: methyl 2-(5-bromofuro[3,2-b]pyridin-3-yl)acetate

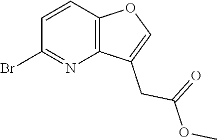

To a solution of ethyl (E)-4-((6-bromo-2-iodopyridin-3-yl)oxy)but-2-enoate (4 g, 9.7 mmol, 1.0 eq.), X-Phos Pd G2 (764 mg, 970 μmol, 0.1 eq.) in DMF (10 mL) was added Na₂CO₃ (2.1 g, 19.4 mmol, 2.0 eq.). The mixture was stirred at 80° C. for 16 hours under N₂ atmosphere. LCMS showed the reaction was completed. The resulting solution was diluted with 200 mL of water, then extracted with EtOAc (3×200 mL) and washed with brine. The combined organic layer was dried over anhydrous sodium sulfate and concentrated under vacuum. The combined organic layer was dried over anhydrous sodium sulfate and concentrated under vacuum. The residue was purified on a silica gel chromatography (100-200 mesh silica gel, 0-30% EtOAc in PE) to give methyl 2-(5-bromofuro[3,2-b]pyridin-3-yl)acetate (800 mg, yield 29%) as a white solid. LC-MS (ESI): mass calcd. for C₁₀HsBrNO₃, 269.0; m/z found, 270.0/272.0 [M+H]⁺.

### Step C: 3-(5-bromofuro[3,2-b]pyridin-3-yl)piperidine-2,6-dione

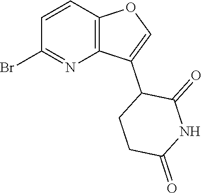 [SMILES: O=C1CCC(c2coc3ccc(Br)nc23)C(=O)N1]

To a solution of methyl 2-(5-bromofuro[3,2-b]pyridin-3-yl)acetate (200 mg, 741 μmol, 1.0 eq.), t-BuOK (83 mg, 741 μmol, 1.0 eq.) in DMF (10 mL) was added acrylamide (53 mg, 741 μmol, 1.0 eq.). The mixture was stirred at 25° C. for 1 hour under N₂ atmosphere. LCMS showed the reaction was completed. The mixture was diluted with sat. NH₄Cl and H₂O, extracted with EtOAc (20 mL×3) and the layers were separated. The organic layer was washed with brine, dried over Na₂SO₄, filtered and concentrated. The product was purified by flash chromatography (100-200 mesh silica gel, 0-80% EtOAc in PE) to afford 3-(5-bromofuro[3,2-b]pyridin-3-yl)piperidine-2,6-dione (60 mg, yield 26%) as a yellow solid. LC-MS (ESI): mass calcd. for C₁₂H₉BrN₂O₃, 308.0; m/z found, 309.0/311.0 [M+H]⁺.

### Intermediate 66. 3-(5-(3-aminoprop-1-yn-1-yl)-1H-indazol-3-yl)piperidine-2,6-dione

### Step A: 5-bromo-3-iodo-1-(tetrahydro-2H-pyran-2-yl)-1H-indazole

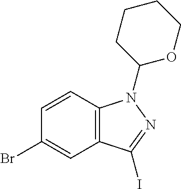 [SMILES: Brc1ccc2c(c1)c(I)nn2C1CCCCO1]

To a solution of 5-bromo-3-iodo-1H-indazole (4 g, 12.4 mmol, 1 eq.), Ts-OH (236 mg, 1.2 mmol, 0.1 eq.) in DCM (40 mL) was added DHP (2.3 mL, 24.8 mmol, 2 eq.) at 0° C. The mixture was stirred at 25° C. for 3 hours, under N₂ atmosphere. The reaction was monitored by LCMS. The resulting solution was diluted with 50 mL of H₂O and extracted with EtOAc (3*40 mL). The combined organic layers were washed with brine, dried over Na₂SO₄ and concentrated under reduced pressure. The residue was purified by flash column chromatography on silica gel (100-200 mesh silica gel, 0-10% EtOAc in PE) to give 5-bromo-3-iodo-1-(tetrahydro-2H-pyran-2-yl)-1H-indazole (4 g, yield 79.3%) as a yellow oil. LC-MS (ESI): mass calcd. for C₁₂H₁₂BrIN₂O, 405.9/407.9; m/z found, 406.9/408.9 [M+H]⁺.

### Step B: tert-butyl 2-(5-bromo-1-(tetrahydro-2H-pyran-2-yl)-1H-indazol-3-yl)-2-cyanoacetate

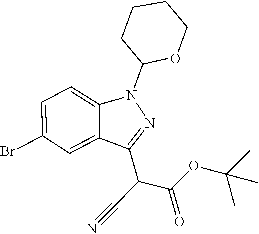 [SMILES: CC(C)(C)OC(=O)C(C#N)c1nn(C2CCCCO2)c2ccc(Br)cc12]

To a solution of 5-bromo-3-iodo-1-(tetrahydro-2H-pyran-2-yl)-1H-indazole (3 g, 7.37 mmol, 1 eq.), CuI (140 mg, 737 μmol, 0.1 eq.) and 1,2-cyclohexanediamine, (lr,2r)-(210 mg, 0.3 mL, 1.8 mmol, 0.25 eq.) in 1,4-Dioxane (40 mL) was added Cs₂CO₃ (7.2 g, 22.1 mmol, 3 eq.) and tert-butyl 2-cyanoacetate (1.9 g, 13.3 mmol, 1.8 eq.). The mixture was stirred at 100° C. for 6 hours, under N₂ atmosphere. The reaction was monitored by LCMS. The resulting solution was diluted with 50 mL of H₂O and extracted with EtOAc (3*40 mL). The combined organic layers were washed with brine, dried over Na₂SO₄ and concentrated under reduced pressure. The residue was purified on silica gel (100-200 mesh silica gel, 0-15% EtOAc in PE) to give tert-butyl 2-(5-bromo-1-(tetrahydro-2H-pyran-2-yl)-1H-indazol-3-yl)-2-cyanoacetate (1.3 g, yield 34%) as a yellow oil. LC-MS (ESI): mass calcd. for C₁₉H₂₂BrN₃O₃, 419.1/421.1; m/z found, 420.1/422.1 [M+H]⁺.

### Step C: 1-(tert-butyl) 5-ethyl 2-(5-bromo-1-(tetrahydro-2H-pyran-2-yl)-1H-indazol-3-yl)-2-cyanopentanedioate

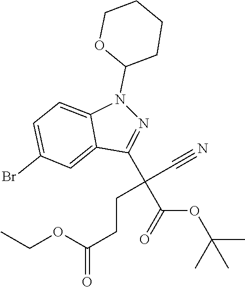 [SMILES: CCOC(=O)CCC(C#N)(C(=O)OC(C)(C)C)c1nn(C2CCCCO2)c2ccc(Br)cc12]

To a solution of tert-butyl 2-(5-bromo-1-(tetrahydro-2H-pyran-2-yl)-1H-indazol-3-yl)-2-cyanoacetate (1.2 g, 2.86 mmol, 1 eq.), benzyl(triethyl)ammonium chloride (65 mg, 286 μmol, 0.1 eq.) in MeCN (15 mL) was added K₂CO₃ (789 mg, 5.71 mmol, 2 eq.) and added ethyl 3-bromopropanoate (1 g, 5.71 mmol, 2 eq.) after 0.5 hour at 88° C. The mixture was stirred at 88° C. for 16 hours, under N₂ atmosphere. The reaction was monitored by LCMS. The resulting solution was diluted with 50 mL of H₂O and extracted with EtOAc (3*40 mL). The combined organic layers were washed with brine, dried over Na₂SO₄ and concentrated under reduced pressure. The residue was purified by flash column chromatography on silica gel (100-200 mesh silica gel, 0-20% EtOAc in PE) to afford 1-(tert-butyl) 5-ethyl 2-(5-bromo-1-(tetrahydro-2H-pyran-2-yl)-1H-indazol-3-yl)-2-cyanopentanedioate (1 g, yield 54%, 80% Purity) as a yellow oil. LC-MS (ESI): mass calcd. for C₂₄H₃₀BrN₃O₅, 519.1/521.1; m/z found, 520.1/522.1 [M+H]⁺.

### Step D: 3-(5-bromo-1H-indazol-3-yl)piperidine-2,6-dione

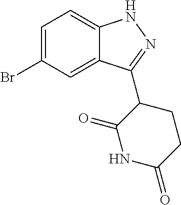 [SMILES: O=C1CCC(c2n[nH]c3ccc(Br)cc23)C(=O)N1]

To a solution of tert-butyl 2-(5-bromo-1-(tetrahydro-2H-pyran-2-yl)-1H-indazol-3-yl)-2-cyanoacetate (1.2 g, 2.86 mmol, 1 eq.) in AcOH (16 mL) was added Sulfuric acid (400 μL).The mixture was stirred at 120° C. for 1 hour, under N₂ atmosphere. The reaction was monitored by LCMS. The resulting solution was diluted with 50 mL of H₂O and extracted with EtOAc (3*40 mL). The combined organic layers were washed with brine, dried over Na₂SO₄ and concentrated under reduced pressure. The residue was purified on silica gel (100-200 mesh silica gel, 0-30% EtOAc in PE) to afford 3-(5-bromo-1H-indazol-3-yl)piperidine-2,6-dione (380 mg, yield 43.2%) as a white solid. LC-MS (ESI): mass calcd. for C₁₂H₁₀BrN₃O₂, 307.0/309.0; m/z found, 308.0/310.0 [M+H]⁺.

### Step E: 3-(5-bromo-1-(tetrahydro-2H-pyran-2-yl)-1H-indazol-3-yl)piperidine-2,6-dione

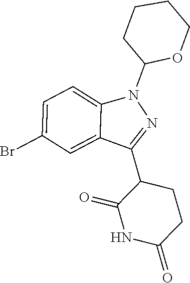 [SMILES: O=C1CCC(c2nn(C3CCCCO3)c3ccc(Br)cc23)C(=O)N1]

To a solution of 3-(5-bromo-1H-indazol-3-yl)piperidine-2,6-dione (120 mg, 389 μmol, 1 eq.), Ts-OH (7.4 mg, 38.9 μmol, 0.1 eq.) in DCM (8 mL) was added DHP (65.5 mg, 71.1 μL, 779 μmol, 2 eq.) at 0° C. The mixture was stirred at 25° C. for 2 hours, under N₂ atmosphere. The reaction was monitored by LCMS. The resulting solution was diluted with 30 mL of H₂O and extracted with EtOAc (3*20 mL). The combined organic layers were washed with brine, dried over Na₂SO₄ and concentrated under reduced pressure. The residue was purified by flash column chromatography on silica gel (100-200 mesh silica gel, 0-30% EtOAc in PE) to afford 3-(5-bromo-1-(tetrahydro-2H-pyran-2-yl)-1H-indazol-3-yl)piperidine-2,6-dione (100 mg, yield 65.5%) as a yellow solid. LC-MS (ESI): mass calcd. For C₁₇H₁₈BrN₃O₃, 391.0/393.0; m/z found, 392.0/394.0 [M+H]⁺.

### Step F: tert-butyl (3-(3-(2,6-dioxopiperidin-3-yl)-1-(tetrahydro-2H-pyran-2-yl)-1H-indazol-5-yl)prop-2-yn-1-yl)carbamate

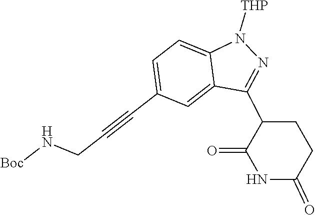

To a solution of 3-(5-bromo-1-(tetrahydro-2H-pyran-2-yl)-1H-indazol-3-yl)piperidine-2,6-dione (100 mg, 255 μmol, 1 eq.), Bis-(triphenylphosphino)-palladous chloride (18 mg, 25.5 μmol, 0.1 eq.), Phosphine, tricyclohexyl-(8 mg, 7.97 μL, 25.5 μmol, 0.1 eq.), CuI (10 mg, 51.0 μmol, 0.2 eq.) in DMF (5 mL) was added TEA (355 μL, 2.55 mmol, 10 eq.) and added tert-butyl prop-2-yn-1-ylcarbamate (119 mg, 765 μmol, 3 eq.) after 10 minutes at 90° C. The mixture was stirred at 90° C. for 3 hours, under N₂ atmosphere. The reaction was monitored by LCMS. The resulting solution was diluted with 30 mL of H₂O and extracted with EtOAc (3*20 mL). The combined organic layers were washed with brine, dried over Na₂SO₄ and concentrated under reduced pressure. The residue was purified by flash column chromatography on silica gel (100-200 mesh silica gel, 0-60% EtOAc in PE) to afford tert-butyl (3-(3-(2,6-dioxopiperidin-3-yl)-1-(tetrahydro-2H-pyran-2-yl)-1H-indazol-5-yl)prop-2-yn-1-yl)carbamate (100 mg, yield 84.1%) as a yellow solid. LC-MS (ESI): mass calcd. for C₂₅H₃₀N₄O₅, 466.2; m/z found, 467.2 [M+H]⁺.

### Step G: 3-(5-(3-aminoprop-1-yn-1-yl)-1H-indazol-3-yl)piperidine-2,6-dione

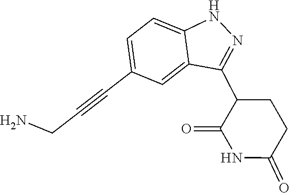 [SMILES: NCC#Cc1ccc2[nH]nc(C3CCC(=O)NC3=O)c2c1]

To a solution of tert-butyl (3-(3-(2,6-dioxopiperidin-3-yl)-1-(tetrahydro-2H-pyran-2-yl)-1H-indazol-5-yl)prop-2-yn-1-yl)carbamate (50 mg, 107 μmol, 1 eq.) in DCM (3 mL) and TFA (600 μL) at 0° C. The mixture was stirred at 25° C. for 6 hours under N₂ atmosphere. The reaction was monitored by LCMS. The reaction was cooled to 20° C. and concentrated under vacuum to give crude 3-(5-(3-aminoprop-1-yn-1-yl)-1H-indazol-3-yl)piperidine-2,6-dione (50 mg, crude) as a brown solid. LC-MS (ESI): mass calcd. for C₁₅H₁₄N₄O₂, 282.1; m/z found, 283.1 [M+H]⁺.

### Intermediate 67. 4-(azetidin-1-yl)-6-chloro-1,3-dimethyl-1,3-dihydro-2H-imidazo[4,5-c]pyridin-2-one

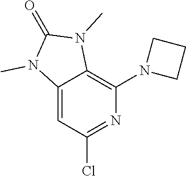 [SMILES: Cn1c(=O)n(C)c2c(N3CCC3)nc(Cl)cc21]

To a solution of 4,6-dichloro-1,3-dimethyl-1,3-dihydro-2H-imidazo[4,5-c]pyridin-2-one (500 mg, 2.2 mmol, 1 eq.) in 1,4-Dioxane (5 mL) was added azetidine (123 mg, 2.2 mmol, 1 eq.), Pd₂(dba)₃ (197 mg, 215 μmol, 0.1 eq.), Xant-Phos (249 mg, 431 μmol, 0.2 eq.) and Cs₂CO₃ (1.4 g, 4.3 mmol, 2 eq.) at 25° C. The mixture was then stirred at 100° C. for 4 hours under N₂ atmosphere. The resulting solution was diluted with 30 mL of water and extracted with EtOAc (30 mL×3). The combined organic layers were washed with brine, dried over Na₂SO₄, and concentrated under reduced pressure. The residue was purified by flash column chromatography on silica gel (100-200 mesh silica gel, 0-50% EtOAc in PE) to give 4-(azetidin-1-yl)-6-chloro-1,3-dimethyl-1,3-dihydro-2H-imidazo[4,5-c]pyridin-2-one (84 mg, 15% yield) as a yellow solid. LC-MS (ESI): mass calcd. for C₁₁H₁₃ClN₄O, 252.1/254.1; m/z found, 252.7/254.7 [M+H]⁺.

### Intermediate 68. 4,6-dichloro-1-ethyl-3-methyl-1,3-dihydro-2H-imidazo[4,5-c]pyridin-2-one

### Step A: 2,6-dichloro-N-ethyl-3-nitropyridin-4-amine

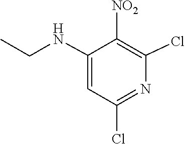

To a mixture of 2,6-dichloro-3-nitropyridin-4-amine (5.0 g, 24.0 mmol, 1 eq.) in DCM (50 mL) and AcOH (12 mL) was added acetaldehyde (7.2 mL, 5.0 μmol, 36.1 mmol, 1.5 eq.) and BMS (13.2 mL, 26.4 mmol, 1.1 eq., 2.0 M in TIF) at 0° C. The mixture was stirred at 25° C. for 3 hours. The mixture was quenched with NaHCO₃ solution and diluted with 50 mL of water and extracted with EtOAc (3* 100 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 2,6-dichloro-N-ethyl-3-nitropyridin-4-amine (1.9 g, yield 33.5%) as a yellow solid. LC-MS (ESI): mass calcd. for C₇H₇Cl₂N₃O₂, 235.0; m/z found, 236.0 [M+H]⁺.

### Step B: 2,6-dichloro-N₄-ethylpyridine-3,4-diamine

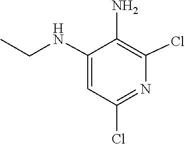

To a mixture of 2,6-dichloro-N-ethyl-3-nitropyridin-4-amine (1.9 g, 8.0 mmol, 1 eq.) in EtOH (20.0 mL) and H₂O(4.0 mL) was added Ammonium chloride (1.7 g, 1.1 mL, 32.2 mmol, 4 eq.) and Iron (3.6 g, 457 μL, 64.4 mmol, 8 eq.). The mixture was stirred at 60° C. for 1 hour. The resulting solution was diluted with 50 mL of water and extracted with EtOAc (3*80 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 2,6-dichloro-N₄-ethylpyridine-3,4-diamine (1.5 g, yield 90.4%) as a yellow solid. LC-MS (ESI): mass calcd. for C₇H₉Cl₂N₃, 205.0; m/z found, 206.0 [M+H]⁺.

### Step C: 4,6-dichloro-1-ethyl-1,3-dihydro-2H-imidazo[4,5-c]pyridin-2-one

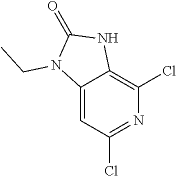

To a mixture of 2,6-dichloro-N₄-ethylpyridine-3,4-diamine (1.9 g, 9.2 mmol, 1 eq.) in THF (20 mL) was added CDI (3.0 g, 18.4 mmol, 2 eq.), and the solution was stirred at 80° C. for 4 hours. The resulting solution was diluted with 50 mL of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 4,6-dichloro-1-ethyl-1,3-dihydro-2H-imidazo[4,5-c]pyridin-2-one (1.5 g, yield 70.1%) as a yellow solid. ¹H NMR (400 MHz, DMSO-d₆) δ 11.89 (s, 1H), 7.51 (s, 1H), 3.84 (d, J=7.2 Hz, 2H), 1.19 (t, J=7.1 Hz, 3H).

### Step D: 4,6-dichloro-1-ethyl-3-methyl-1,3-dihydro-2H-imidazo[4,5-c]pyridin-2-one

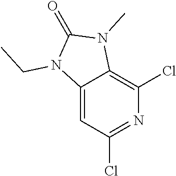

To a solution of 4,6-dichloro-1-ethyl-1,3-dihydro-2H-imidazo[4,5-c]pyridin-2-one (1.5 g, 6.4 mmol, 1 eq.) in DMF (15 mL) was added NaH (186 mg, 7.7 mmol, 1.2 eq.) at 0° C., the reaction was stirred at 0° C. for 30 min under N₂ atmosphere. Then the mixture was added Mel (445 μL, 7.1 mmol, 1.1 eq.) at 0° C., the reaction was stirred at 25° C. for 1 hour under N₂ atmosphere. The resulting solution was diluted with 50 mL of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 4,6-dichloro-1-ethyl-3-methyl-1,3-dihydro-2H-imidazo[4,5-c]pyridin-2-one (1.3 g, yield 81.7%) as a white solid. LC-MS (ESI): mass calcd. for C₉H₉C₁₂N₃O, 245.0; m/z found, 246.0 [M+H]⁺.

### Intermediate 69. Methyl 5-(5-amino-4-methoxypyrimidin-2-yl)-3-methylpicolinate

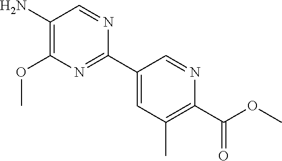 [SMILES: COC(=O)c1ncc(-c2ncc(N)c(OC)n2)cc1C]

A solution of 2-chloro-4-methoxypyrimidin-5-amine (400 mg, 2.5 mmol, 1.0 eq.), Pd(dppf)Cl₂ (163 mg, 251 μmol, 0.1 eq.), methyl 3-methyl-5-(4,4,5,5-tetramethyl-1,3,2-dioxaborolan-2-yl)picolinate (834 mg, 3.0 mmol, 1.2 eq.), K₃PO₄ (1.6 g, 7.5 mmol, 3.0 eq.) in 1,4-Dioxane (10 mL) and Water (1 mL) was stirred at 100° C. for 1 hour under N₂. After cooled to room temperature, the resulting solution was diluted with 30 mL of water and extracted with EtOAc (10 mL×3). The combined organic layers were washed with brine (10 mL), dried over Na₂SO₄ and concentrated under reduced pressure. The residue was purified by flash column chromatography on silica gel (100-200 mesh silica gel, 0-100% EtOAc in PE) to give methyl 5-(5-amino-4-methoxypyrimidin-2-yl)-3-methylpicolinate (240 mg, 35% yiled) as a brown solid. LC-MS (ESI): mass calcd. for C₁₃H₁₄N₄O₃, 274.11; m/z found, 275 [M+H]⁺.

### Intermediate 70. 3-(2-iodothieno[3,2-c]pyridin-4-yl)piperidine-2,6-dione

### Step A: tert-butyl 2-cyano-2-(2-iodothieno[3,2-c]pyridin-4-yl)acetate

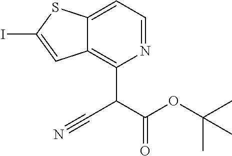 [SMILES: CC(C)(C)OC(=O)C(C#N)c1nccc2sc(I)cc12]

To a mixture of 4-chloro-2-iodothieno[3,2-c]pyridine (6.0 g, 20.3 mmol, 1 eq.) in DMSO (60 mL) was added K₂CO₃ (8.4 g, 60.9 mmol, 3 eq.) and tert-butyl 2-cyanoacetate (2.9 g, 20.3 mmol, 1 eq.), and the solution was stirred at 120° C. for 3 hours. The resulting solution was diluted with 150 mL of water and extracted with EtOAc (3* 150 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford tert-butyl 2-cyano-2-(2-iodothieno[3,2-c]pyridin-4-yl)acetate (2.1 g, yield 25.8%) as a yellow solid. LC-MS (ESI): mass calcd. for C₁₄H₁₃IN₂O₂S, 400.0; m/z found, 401.0 [M+H]⁺.

### Step B: 2-(2-iodothieno[3,2-c]pyridin-4-yl)acetonitrile

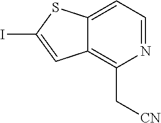 [SMILES: N#CCc1nccc2sc(I)cc12]

To a solution of tert-butyl 2-cyano-2-(2-iodothieno[3,2-c]pyridin-4-yl)acetate (2.1 g, 5.2 mmol) in DCM (3 mL) was added TFA (3 mL) and stirred at room temperature for 8 hours. The resulting solution was diluted with 100 mL of water and extracted with EtOAc (3*100 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 2-(2-iodothieno[3,2-c]pyridin-4-yl)acetonitrile (1.0 g, yield 66%) as a yellow solid. LC-MS (ESI): mass calcd. for C₉H₅IN₂S, 300.0; m/z found, 301.0 [M+H]⁺.

### Step C: ethyl 4-cyano-4-(2-iodothieno[3,2-c]pyridin-4-yl)butanoate

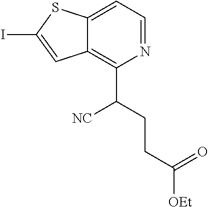 [SMILES: CCOC(=O)CCC(C#N)c1nccc2sc(I)cc12]

To the solution of 2-(2-iodothieno[3,2-c]pyridin-4-yl)acetonitrile (330 mg, 1.1 mmol, 1 eq.) and K₂CO₃ (456 mg, 3.3 mmol, 3 eq.) in DMF (5 mL) was added ethyl 3-bromopropanoate (199 mg, 1.1 mmol, 1 eq.) at 25° C. The mixture was stirred at 50° C. for 30 min under N₂ atmosphere. The resulting solution was diluted with 50 mL of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford the ethyl 4-cyano-4-(2-iodothieno[3,2-c]pyridin-4-yl)butanoate (160 mg, yield 36.4%) as a yellow oil. LC-MS (ESI): mass calcd. for C₁₄H₁₃IN₂O₂S, 400.0; m/z found, 401.0 [M+H]⁺.

### Step D: 3-(2-iodothieno[3,2-c]pyridin-4-yl)piperidine-2,6-dione

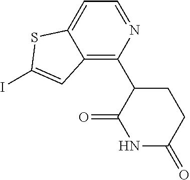 [SMILES: O=C1CCC(c2nccc3sc(I)cc23)C(=O)N1]

To the solution of ethyl 4-cyano-4-(2-iodothieno[3,2-c]pyridin-4-yl)butanoate (160 mg, 400 μmol, 1 eq.) in AcOH (4 mL) was added con. H₂SO₄ (0.4 mL) at 25° C. The mixture was stirred at 100° C. for 1 hour under N₂ atmosphere. The resulting solution was diluted with 50 mL of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford the product 3-(2-iodothieno[3,2-c]pyridin-4-yl)piperidine-2,6-dione (120 mg, yield 80.7%) as a white solid. LC-MS (ESI): mass calcd. for C₁₂H₉IN₂O₂S, 371.9; m/z found, 373.0 [M+H]⁺.

### Intermediate 70. 3-(6-bromo-1H-indazol-1-yl)piperidine-2,6-dione

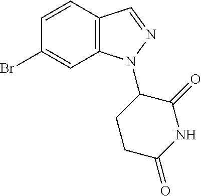 [SMILES: O=C1CCC(n2ncc3ccc(Br)cc32)C(=O)N1]

To a solution of 6-bromo-1H-indazole (3.5 g, 17.8 mmol, 1.0 eq.) in THF (40 mL) was added 3-bromopiperidine-2,6-dione (5.1 g, 26.6 mmol, 1.5 eq.) and t-BuOK (5.9 g, 53.3 mmol, 3.0 eq.), the solution was stirred at 50° C. for 16 hours to give purple solution. LCMS showed the reaction was completed. The resulting solution was diluted with 200 mL of water and extracted with EtOAc (3×200 mL). The combined organic layers were washed with brine (3×200 mL), dried over Na₂SO₄, and concentrated under reduced pressure. The residue was purified by flash chromatography (100-200 mesh silica gel, 0% ˜50% EtOAC in PE) to afford 3-(6-bromo-1H-indazol-1-yl)piperidine-2,6-dione (240 mg, yield 4.2%) as a yellow solid. LC-MS (ESI): mass calcd. for C₁₂H₁₀BrN₃O₂, 307.0/309.0; m/z found, 308.0/310.0 [M+H]⁺.

### Intermediate 71. 1,3-dimethyl-7-(1-methylpiperidin-4-yl)-2-oxo-2,3-dihydro-1H-benzo[d]imidazol-5-yl trifluoromethanesulfonate

### Step A: 4-bromo-6-hydroxy-1,3-dimethyl-1,3-dihydro-2H-benzo[d]imidazol-2-one

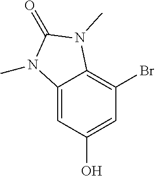

To a solution of 4-bromo-6-methoxy-1,3-dimethyl-1,3-dihydro-2H-benzo[d]imidazol-2-one (600 mg, 2.2 mmol, 1.0 eq.) in DCM (15 mL) was added Boron tribromide (3.3 g, 13.3 mmol, 6.0 eq.) at 0° C. The mixture was stirred at 25° C. for 12 hours under N₂ atmosphere. The reaction was monitored by LCMS. The reaction mixture was quenched with 10 ml of MeOH. The solid was collected by filtration to afford 4-bromo-6-hydroxy-1,3-dimethyl-1,3-dihydro-2H-benzo[d]imidazol-2-one (320 mg, yield 56%) as a white solid. LC-MS (ESI): mass calcd. for C₉H₉BrN₂O₂, 256.1; m/z found, 257.3 [M+H]⁺.

### Step B: 6-hydroxy-1,3-dimethyl-4-(1-methyl-1,2,3,6-tetrahydropyridin-4-yl)-1,3-dihydro-2H-ben zo[d]imidazol-2-one

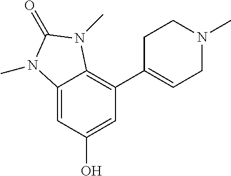

To a solution of 4-bromo-6-hydroxy-1,3-dimethyl-1,3-dihydro-2H-benzo[d]imidazol-2-one (320 mg, 1.2 mmol, 1.0 eq.), Na₂CO₃ (396 mg, 3.7 mmol, 3.0 eq.) and 1-methyl-4-(4,4,5,5-tetramethyl-1,3,2-dioxaborolan-2-yl)-1,2,3,6-tetrahydropyridine (417 mg, 1.5 mmol, 1.9 eq.) in 1,4-Dioxane (5 mL) and H₂O(0.5 mL) was added Pd(dppf)Cl₂ (91 mg, 0.1 mmol, 0.1 eq.) at 25° C. The mixture was stirred at 120° C. for 2 hours under N₂ atmosphere. The reaction was monitored by LCMS. The resulting solution was diluted with 20 ml of water and extracted with EtOAc (3*20 mL). The mixture was filtered and the filtrate was concentrated under vacuum and purified by flash column chromatography on silica gel (100-200 mesh silica gel, 0-10% MeOH in DCM) to afford 6-hydroxy-1,3-dimethyl-4-(1-methyl-1,2,3,6-tetrahydropyridin-4-yl)-1,3-dihydro-2H-benzo[d]imidazol-2-one (100 mg, yield 29%) as a brown oil. LC-MS (ESI): mass calcd. for C₁₅H₁₉N₃O₂, 273.1; m/z found, 274.4 [M+H]⁺.

### Step C: 6-hydroxy-1,3-dimethyl-4-(1-methylpiperidin-4-yl)-1,3-dihydro-2H-benzo[d]imidazol-2-one

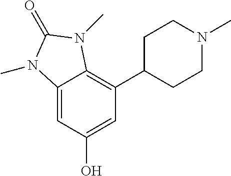

To a solution of 6-hydroxy-1,3-dimethyl-4-(1-methyl-1,2,3,6-tetrahydropyridin-4-yl)-1,3-dihydro-2H-benzo[d]imidazol-2-one (100 mg, 0.4 mmol, 1.0 eq.) in MeOH (5 mL) was added Pd/C (50 mg, w.t. =10%), and Pd(OH)₂/C (50 mg, w.t. =10%). The mixture was degassed with H₂ for 3 times and stirred at 50° C. using a H₂ balloon for 18 hours. The resulting solution was filtered through a Celite pad and filtrate was concentrated under vacuum to give 3-((3-fluoro-4-hydroxypyridin-2-yl)amino)piperidine-2,6-dione (80 mg, crude) which was used for next step directly without further purification. LC-MS (ESI): mass calcd. for C₁₅H₂₁N₃O₂, 275.1; m/z found, 276.4 [M+H]⁺.

### Step D: 1,3-dimethyl-7-(1-methylpiperidin-4-yl)-2-oxo-2,3-dihydro-1H-benzo[d]imidazol-5-yl tr ifluoromethanesulfonate

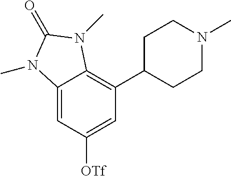

To a solution of 6-hydroxy-1,3-dimethyl-4-(1-methylpiperidin-4-yl)-1,3-dihydro-2H-benzo[d]imidazol-2-one (80 mg, 1.2 mmol, 1.0 eq.) and TEA (0.1 mL, 0.9 mmol, 3.0 eq.) in DCM (5 mL) was added Trifluoroaceticanhydride (92 mg, 0.4 mmol, 1.5 eq.) at 0° C. The mixture was stirred at 25° C. for 1 hour under N₂ atmosphere. The reaction was monitored by LCMS. The resulting solution was diluted with 10 ml of water and extracted with DCM (3* 10 mL). The mixture was filtered and the filtrate was concentrated under vacuum and purified by flash column chromatography on silica gel (100-200 mesh silica gel, 0-10% MeOH in DCM) to afford 1,3-dimethyl-7-(1-methylpiperidin-4-yl)-2-oxo-2,3-dihydro-1H-benzo[d]imidazol-5-yl trifluoromethanesulfonate (75 mg, yield 63%) as a yellow solid. LC-MS (ESI): mass calcd. for C₁₆H₂₀FN₃O₄S, 407.1; m/z found, 408.4 [M+H]⁺.

### Intermediate 72. 3-methyl-2-oxo-7-(tetrahydro-2H-pyran-4-yl)-2,3-dihydrobenzo[d]oxazol-5-yl trifluoromethanesulfonate

### Step A: 2-amino-6-bromo-4-methoxyphenol

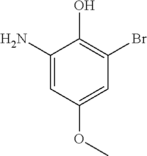

To a solution of 2-bromo-4-methoxy-6-nitrophenol (4.5 g, 18.1 mmol, 1.0 eq.) in EtOH (30 mL) and Water (30 mL) was added NH₄Cl (9.7 g, 181 mmol, 10.0 eq.) and iron (10.1 g, 181 mmol, 10.0 eq.). The solution was stirred at 80° C. for 3 hours to give black solution. LCMS showed the reaction was completed. The resulting solution was diluted with 100 mL of water and extracted with EtOAc (3×100 mL). The combined organic layers were washed with brine (3×100 mL), dried over Na₂SO₄, and concentrated under reduced pressure. The residue was purified by flash chromatography (100-200 mesh silica gel, 0% ˜100% EtOAc in PE) to afford 2-amino-6-bromo-4-methoxyphenol (2.4 g, yield 58%) as a yellow solid. LC-MS (ESI): mass calcd. for C₇H₈BrNO₂, 217.0/219.0; m/z found, 218.0/220.0 [M+H]⁺.

### Step B: 7-bromo-5-methoxybenzo[d]oxazol-2(3H)-one

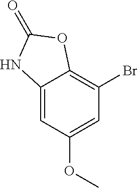

To a solution of 2-amino-6-bromo-4-methoxyphenol (2.4 g, 11 mmol, 1.0 eq.) in THF (30 mL) was added CDI (3.6 g, 22 mmol, 2.0 eq.). The solution was stirred at 70° C. for 3 hours to give brown solution. LCMS showed the reaction was completed. The resulting solution was diluted with 100 mL of water and extracted with EtOAc (3×50 mL). The combined organic layers were washed with brine (3×50 mL), dried over Na₂SO₄, and concentrated under reduced pressure. The residue was purified by flash chromatography (100-200 mesh silica gel, 0% ˜100% EtOAC in PE) to afford 7-bromo-5-methoxybenzo[d]oxazol-2(3H)-one (2.3 g, yield 81%) as a yellow solid. LC-MS (ESI): mass calcd. for C₈H₆BrNO₃, 243.0/245.0; m/z found, 244.0/246.0 [M+H]⁺.

### Step C: 7-bromo-5-methoxy-3-methylbenzo[d]oxazol-2(3H)-one

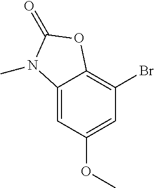

To a solution of 7-bromo-5-methoxybenzo[d]oxazol-2(3H)-one (2.3 g, 9.7 mmol, 1.0 eq.) in DMF (40 mL) was added Mel (1.8 mL, 29.3 mmol, 3.0 eq.) and K₂CO₃ (8.1 g, 58.5 mmol, 6.0 eq.). The solution was stirred at 25° C. for 1 hour to give brown solution. LCMS showed the reaction was completed. The solution was quenched water (500 mL), filtered and concentrated to afford 7-bromo-5-methoxy-3-methylbenzo[d]oxazol-2(3H)-one (2.2 g, yield 83%) as a yellow solid. LC-MS (ESI): mass calcd. for C₉H₈BrNO₃, 257.0/259.0; m/z found, 258.0/260.0 [M+H]⁺.

### Step D: 7-(3,6-dihydro-2H-pyran-4-yl)-5-methoxy-3-methylbenzo[d]oxazol-2(3H)-one

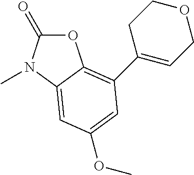 [SMILES: COc1cc(C2=CCOCC2)c2oc(=O)n(C)c2c1]

To a solution of 2-(3,6-dihydro-2H-pyran-4-yl)-4,4,5,5-tetramethyl-1,3,2-dioxaborolane (2.9 g, 13.9 mmol, 3.0 eq.) in 1,4-Dioxane (20 mL) and Water (2 mL) was added 7-bromo-5-methoxy-3-methylbenzo[d]oxazol-2(3H)-one (1.2 g, 4.6 mmol, 1.0 eq.), Na₂CO₃ (1.5 g, 13.9 mmol, 3.0 eq.) and Pd(dppf)Cl₂ (340 mg, 465 μmol, 0.1 eq.) under N₂. The solution was stirred at 100° C. for 5 hours to give black solution. LCMS showed the reaction was completed. The resulting solution was diluted with 100 mL of water and extracted with EtOAc (3×100 mL). The combined organic layers were washed with brine (3×100 mL), dried over Na₂SO₄, and concentrated under reduced pressure. The residue was purified by flash chromatography (100-200 mesh silica gel, 0% ˜100% EtOAc in PE) to afford 7-(3,6-dihydro-2H-pyran-4-yl)-5-methoxy-3-methylbenzo[d]oxazol-2(3H)-one (700 mg, yield 55%) as a yellow solid. LC-MS (ESI): mass calcd. for C₉H₈BrNO₃, 261.1; m/z found, 262.1 [M+H]⁺.

### Step E: 5-methoxy-3-methyl-7-(tetrahydro-2H-pyran-4-yl)benzo[d]oxazol-2(3H)-one

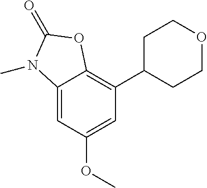 [SMILES: COc1cc(C2CCOCC2)c2oc(=O)n(C)c2c1]

To a solution of 7-(3,6-dihydro-2H-pyran-4-yl)-5-methoxy-3-methylbenzo[d]oxazol-2(3H)-one (750 mg, 2.9 mmol, 1.0 eq.) in MeOH (20 mL) was added Pd/C (400 mg, 3.7 mmol, 1.3 eq.) under H₂. The reaction solution was stirred at 50° C. using a H₂ balloon for 4 hours to give black solution. LCMS showed the reaction was completed. The solution was filtered and concentrated to afford 5-methoxy-3-methyl-7-(tetrahydro-2H-pyran-4-yl)benzo[d]oxazol-2(3H)-one (650 mg, yield 82%) as a yellow solid. LC-MS (ESI): mass calcd. for C₁₄H₁₇NO₄, 263.1; m/z found, 264.1 [M+H]⁺.

### Step F: 5-hydroxy-3-methyl-7-(tetrahydro-2H-pyran-4-yl)benzo[d]oxazol-2(3H)-one

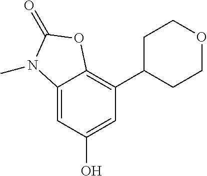 [SMILES: Cn1c(=O)oc2c(C3CCOCC3)cc(O)cc21]

To a solution of 5-methoxy-3-methyl-7-(tetrahydro-2H-pyran-4-yl)benzo[d]oxazol-2(3H)-one (300 mg, 1.1 mmol, 1.0 eq.) in DCM (10 mL) was added BBr₃ (539 μL, 5.7 mmol, 5.0 eq.). The solution was stirred at 0° C. for 1 hour to give yellow solution. LCMS showed the reaction was completed. The solution was quenched with MeOH, extracted with DCM (50 mL) and the layers were separated. The organic layer was washed with brine (50 mL), dried over Na₂SO₄, filtered and concentrated. The product was purified by flash chromatography (100-200 mesh silica gel, 0% ˜100% EtOAC in PE) to afford 5-hydroxy-3-methyl-7-(tetrahydro-2H-pyran-4-yl)benzo[d]oxazol-2(3H)-one (36 mg, yield 12%) as a yellow solid. LC-MS (ESI): mass calcd. for C₁₃H₁₅NO₄, 249.1; m/z found, 250.1 [M+H]⁺.

### Step G: 3-methyl-2-oxo-7-(tetrahydro-2H-pyran-4-yl)-2,3-dihydrobenzo[d]oxazol-5-yl trifluoromethanesulfonate

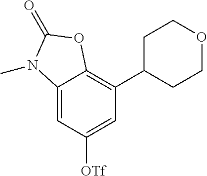 [SMILES: Cn1c(=O)oc2c(C3CCOCC3)cc(OS(=O)(=O)C(F)(F)F)cc21]

To a solution of 5-hydroxy-3-methyl-7-(tetrahydro-2H-pyran-4-yl)benzo[d]oxazol-2(3H)-one (45 mg, 181 μmol, 1.0 eq.) in DCM (5 mL) was added Tf₂O (77 mg, 271 μmol, 1.5 eq.) and TEA (50 μL, 361 μmol, 2.0 eq.) at 0° C. The solution was stirred at 25° C. for 1 hour to give yellow solution. LCMS showed the reaction was completed. The solution was quenched water, extracted with DCM (50 mL) and the layers were separated. The organic layer was washed with brine (50 mL), dried over Na₂SO₄, filtered and concentrated. The product was purified by flash chromatography (100-200 mesh silica gel, 0% ˜100% EtOAc in PE) to afford 3-methyl-2-oxo-7-(tetrahydro-2H-pyran-4-yl)-2,3-dihydrobenzo[d]oxazol-5-yl trifluoromethanesulfonate (50 mg, yield 69%) as a yellow solid. LC-MS (ESI): mass calcd. for C₁₄H₁₄F₃NO₆S, 381.0; m/z found, 382.0 [M+H]⁺.

### Intermediate 73. 3-(5-iodobenzo[d]isoxazol-3-yl)piperidine-2,6-dione

### Step A: 4-hydroxy-6-iodo-2H-chromen-2-one

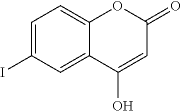 [SMILES: O=c1cc(O)c2cc(I)ccc2o1]

To a solution of 1-(2-hydroxy-5-iodophenyl)ethan-1-one (800 mg, 3.1 mmol, 1.0 eq.), and dimethyl carbonate (825 mg, 9.3 mmol, 3.0 eq.) in THF (30 mL) was added t-BuOk (2.4 g, 21.4 mmol, 7.0 eq.) at 0° C. The mixture was stirred for 16 hours at 70° C. The resulting solution was diluted with 50 mL of water and extracted with EtOAc (3×20 mL) and washed with brine (20 mL). The combined organic layers were dried over anhydrous Na₂SO₄ and concentrated under reduced pressure. The residue was purified on a silica gel chromatography (100-200 mesh silica gel, 0-100% EtOAc in PE) to give 4-hydroxy-6-iodo-2H-chromen-2-one (800 mg, 91% yield) as a white solid. LC-MS (ESI): mass calcd. for C₉H₅IO³, 287.9; m/z found, 288.9 [M+H]⁺.

### Step B: 2-(5-iodobenzo[d]isoxazol-3-yl)acetic acid

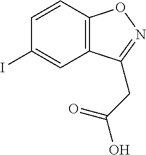 [SMILES: O=C(O)Cc1noc2ccc(I)cc12]

To a solution of 4-hydroxy-6-iodo-2H-chromen-2-one (800 mg, 2.8 mmol, 1.0 eq.) and Sodium acetate (800 mg, 9.8 mmol, 3.5 eq.) in EtOH (20 mL) was added hydroxylamine (322 mg, 9.8 mmol, 3.5 eq.) at room temperature. The reaction mixture was stirred at room temperature for 16 hours. The mixture was filtered and the organic was concentrated by vacuum, 2N HCl was added to adjust pH=2 and extracted with EtOAc (3×20 mL) and washed with brine (20 mL). The combined organic layers were dried over anhydrous Na₂SO₄ and concentrated under reduced pressure. The residue was purified on a silica gel chromatography (100-200 mesh silica gel, 0-100% EtOAc in PE) to give 2-(5-iodobenzo[d]isoxazol-3-yl)acetic acid (300 mg, 36% yield) as a white solid. LC-MS (ESI): mass calcd. for C₉H₆INO₃, 302.9; m/z found, 303.9 [M+H]⁺.

### Step C: ethyl 2-(5-iodobenzo[d]isoxazol-3-yl)acetate

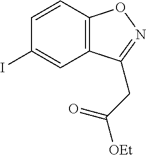 [SMILES: CCOC(=O)Cc1noc2ccc(I)cc12]

To a solution of 2-(5-iodobenzo[d]isoxazol-3-yl)acetic acid (300 mg, 1 mmol, 1.0 eq.) in EtOH (10 mL) was added con. H₂SO₄ (16 μL, 0.3 mmol, 0.3 eq.) at room temperature. The reaction mixture was stirred at room temperature for 16 hours. The resulting solution was diluted with 20 mL of water and extracted with EtOAc (3×20 mL) and washed with brine (20 mL). The combined organic layers were dried over anhydrous Na₂SO₄ and concentrated under reduced pressure. The residue was purified on a silica gel chromatography (100-200 mesh silica gel, 0-100% EtOAc in PE) to give 4-hydroxy-6-iodo-2H-chromen-2-one (300 mg, 91% yield) as a white solid. LC-MS (ESI): mass calcd. for C₁₁H₁₀INO₃, 331.0; m/z found, 332.0 [M+H]⁺.

### Step D: 3-(5-iodobenzo[d]isoxazol-3-yl)piperidine-2,6-dione

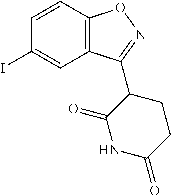 [SMILES: O=C1CCC(c2noc3ccc(I)cc23)C(=O)N1]

To a solution of ethyl 2-(5-iodobenzo[d]isoxazol-3-yl)acetate (200 mg, 604 μmol, 1.0 eq.) and acrylamide (52 mg, 724 μmol, 1.2 eq.) in THF (8 mL) was added t-BuOK (81 mg, 724 μmol, 1.2 eq.). The reaction mixture was stirred at room temperature for 1 hour. The resulting solution was diluted with 20 mL of water and extracted with EtOAc (3×20 mL) and washed with brine (20 mL). The combined organic layers were dried over anhydrous Na₂SO₄ and concentrated under reduced pressure. The residue was purified on a silica gel chromatography (100-200 mesh silica gel, 0-100% EtOAc in PE) to give 3-(5-iodobenzo[d]isoxazol-3-yl)piperidine-2,6-dione (100 mg, 31% yield) as a white solid. LC-MS (ESI): mass calcd. for C₁₂H₉IN₂O₃, 356.0; m/z found, 357.0 [M+H]⁺.

### Intermediate 74. 3-(6-bromoquinazolin-4-yl)piperidine-2,6-dione

### Step A: ethyl 2-(6-bromoquinazolin-4-yl)acetate

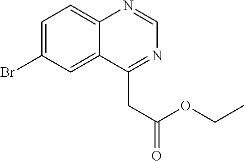 [SMILES: CCOC(=O)Cc1ncnc2ccc(Br)cc12]

To a solution of NaH (1 g, 43.1 mmol, 1.5 eq., w.t.=60% in oil) in THF (50 mL) was added ethyl 3-oxobutanoate (7.5 g, 57.5 mmol, 2.0 eq.) at 0° C. under N₂ atmosphere. The mixture was then stirred at 0° C. for 10 mins. Then 6-bromo-4-chloroquinazoline (7 g, 28.7 mmol, 1.0 eq.) and Toluene (50 mL) was added to the reaction. The mixture was then stirred at 110° C. for 6 hours. The resulting solution was diluted with 50 ml of water and extracted with EtOAc (50 mL×3). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford ethyl 2-(6-bromoquinazolin-4-yl)acetate (4.5 g, yield 53%) as a white solid. LC-MS (ESI): mass calcd. for C₁₂H₁₁BrN₂O₂, 294.0; m/z found, 295.0/297.0 [M+H]⁺.

### Step B: ethyl 2-(6-bromoquinazolin-4-yl)-4-cyanobutanoate

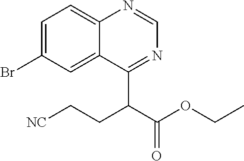 [SMILES: CCOC(=O)C(CCC#N)c1ncnc2ccc(Br)cc12]

To a solution of ethyl 2-(6-bromoquinazolin-4-yl)acetate (1.5 g, 5.1 mmol, 1.0 eq.) in t-BuOH (30 mL) was added acrylonitrile (162 mg, 3.1 mmol, 0.6 eq.) and benzyl(trimethyl)ammonium hydroxide (170 mg, 1.0 mmol, 0.2 eq.). The reaction was stirred at 25° C. for 2 hours. The resulting solution was diluted with 50 ml of water and extracted with EtOAc (50 mL×3). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford ethyl 2-(6-bromoquinazolin-4-yl)-4-cyanobutanoate (290 mg, yield 15%) as a white solid. LC-MS (ESI): mass calcd. for C₁₅H₁₄BrN₃O₂, 347.0; m/z found, 248.0/250.0 [M+H]⁺.

### Step C: ethyl 5-amino-2-(6-bromoquinazolin-4-yl)-5-oxopentanoate

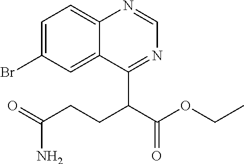 [SMILES: CCOC(=O)C(CCC(N)=O)c1ncnc2ccc(Br)cc12]

To a solution of ethyl 2-(6-bromoquinazolin-4-yl)-4-cyanobutanoate (290 mg, 833 μmol, 1.0 eq.) in Toluene (10 mL) was added Acetaldoxime (148 mg, 2.5 mmol, 3.0 eq.) and Indium trichloride (18 mg, 83.3 μmol, 0.1 eq.). The reaction was stirred at 110° C. for 1 hour. The resulting solution was diluted with 50 ml of water and extracted with EtOAc (50 mL×3). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford ethyl 5-amino-2-(6-bromoquinazolin-4-yl)-5-oxopentanoate (200 mg, yield 66%) as a white solid.

LC-MS (ESI): mass calcd. for C₁₅H₁₆BrN₃O₃, 365.0; m/z found, 366.0/368.0 [M+H]⁺.

### Step D: 3-(6-bromoquinazolin-4-yl)piperidine-2,6-dione

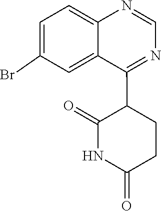 [SMILES: O=C1CCC(c2ncnc3ccc(Br)cc23)C(=O)N1]

To a solution of ethyl 5-amino-2-(6-bromoquinazolin-4-yl)-5-oxopentanoate (175 mg, 478 μmol, 1.0 eq.) in MeCN (8 mL) was added benzyl(trimethyl)ammonium hydroxide (240 mg, 1.4 mmol, 3.0 eq.). The reaction was stirred at 60° C. for 1 hour. The resulting solution was diluted with 50 ml of water and extracted with EtOAc (50 mL×3). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 3-(6-bromoquinazolin-4-yl)piperidine-2,6-dione (105 mg, yield 69%) as a white solid. LC-MS (ESI): mass calcd. for C₁₃H₁₀BrN₃O₂, 319.0; m/z found, 320.0/322.0 [M+H]⁺.

### Intermediate 75. 5-(3-chloro-6,7-dihydropyrido[2,3-c]pyridazin-8(5H)-yl)-1,3-dimethyl-1,3-dihydro-2H-benzo[d]imidazol-2-one

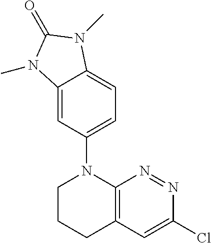 [SMILES: Cn1c(=O)n(C)c2cc(N3CCCc4cc(Cl)nnc43)ccc21]

To a solution of 5-bromo-1,3-dimethyl-1,3-dihydro-2H-benzo[d]imidazol-2-one (200 mg, 415 μmol, 1.0 eq.) in Toluene (10 mL) was added Xant-Phos (35 mg, 83 μmol, 0.2 eq.), 3-chloro-5,6,7,8-tetrahydropyrido[2,3-c]pyridazine (71 mg, 415 μmol, 1.0 eq.), Pd₂(dba)₃ (38 mg, 4 μmol, 0.1 eq.)and t-BuONa (14 mg, 124 μmol, 3.0 eq.) under N₂. The mixture was stirred at 110° C. for 4 hours to give brown solution. LCMS showed the reaction was completed. The resulting solution was diluted with 50 mL of water and extracted with EtOAc (3×50 mL). The combined organic layers were washed with brine (50 mL), dried over Na₂SO₄, and concentrated under reduced pressure. The residue was purified by flash chromatography (100-200 mesh silica gel, 0% ˜100% EtOAC in PE) to afford 3 5-(3-chloro-6,7-dihydropyrido[2,3-c]pyridazin-8(5H)-yl)-1,3-dimethyl-1,3-dihydro-2H-benzo[d]imidazol-2-one (100 mg, yield 30%) as a yellow solid. LC-MS (ESI): mass calcd. for C₁₆H₁₆ClN₅O, 329.3; m/z found, 330.3 [M+H]⁺.

### Intermediate 76. methyl 5-(6-fluoro-4-(7-(3-methoxyazetidin-1-yl)-1,3-dimethyl-2-oxo-1,2-dihydroquinolin-5-yl)-3,4-dihydro-2H-benzo[b][1,4]oxazin-7-yl)picolinate

### Step A: 5-bromo-7-(3-methoxyazetidin-1-yl)-1,3-dimethylquinolin-2(1H)-one

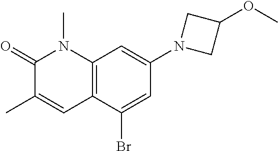

To a solution of 5-bromo-1,3-dimethyl-2-oxo-1,2-dihydroquinolin-7-yl trifluoromethanesulfonate (300 mg, 750 μmol, 1.0 eq.) in Toluene (10 mL) was added Pd₂(dba)₃ (69 mg, 75 μmol, 0.1 eq.), 3-Methoxy-Azetidine Hydrochloride (93 mg, 750 μmol, 1.0 eq.), Xant-Phos (87 mg, 150 μmol, 0.2 eq.) and Cs₂CO₃ (733 mg, 2.3 mmol, 3.0 eq.). The reaction was stirred at 90° C. for 3 hours under N₂. The resulting solution was diluted with 50 mL of water and extracted with EtOAc (3×50 mL). The combined organic layers were washed with brine (50 mL), dried over Na₂SO₄, and concentrated under reduced pressure. The residue was purified by flash chromatography (100-200 mesh silica gel, 0% ˜100% EtOAC in PE) to afford 5-bromo-7-(3-methoxyazetidin-1-yl)-1,3-dimethylquinolin-2(1H)-one (50 mg, yield 19%) as a yellow solid. LC-MS (ESI): mass calcd. for C₁₅H₁₇BrN₂O₂, 336.0/338.0; m/z found, 337.0/339.0 [M+H]⁺.

### Step B: methyl 5-(6-fluoro-4-(7-(3-methoxyazetidin-1-yl)-1,3-dimethyl-2-oxo-1,2-dihydroquinolin-5-yl)-3,4-dihydro-2H-benzo[b][1,4]oxazin-7-yl)picolinate

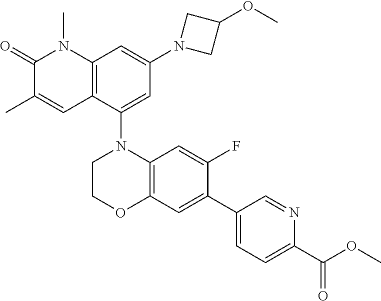

To a solution of methyl 5-(6-fluoro-3,4-dihydro-2H-benzo[b][1,4]oxazin-7-yl)picolinate (89 mg, 311 μmol, 1.5 eq.) in 1,4-Dioxane (2 mL) was added 5-bromo-7-(3-methoxyazetidin-1-yl)-1,3-dimethylquinolin-2(1H)-one (70 mg, 208 μmol, 1.0 eq.), Xant-Phos (24 mg, 42 μmol, 0.2 eq.), Pd₂(dba)₃ (19 mg, 21 μmol, 0.1 eq.) and Cs₂CO₃ (203 mg, 623 μmol, 3.0 eq.) under N₂. The mixture was stirred at 100° C. for 5 hours to give black solution. LCMS showed the reaction was completed. The resulting solution was diluted with 50 mL of water and extracted with EtOAc (3×50 mL). The combined organic layers were washed with brine (50 mL), dried over Na₂SO₄, and concentrated under reduced pressure. The residue was purified by flash chromatography (100-200 mesh silica gel, 0% ˜100% EtOAC in PE) to afford methyl 5-(6-fluoro-4-(7-(3-methoxyazetidin-1-yl)-1,3-dimethyl-2-oxo-1,2-dihydroquinolin-5-yl)-3,4-dihydro-2H-benzo[b][1,4]oxazin-7-yl)picolinate (50 mg, yield 42%) as a yellow solid. LC-MS (ESI): mass calcd. for C₃₀H₂₉FN₄O₅, 544.2; m/z found, 545.2 [M+H]⁺.

### Intermediate 77. 1-(tert-butyl) 5-ethyl 2-(5-bromo-1-methyl-1H-pyrazolo[3,4-b]pyridin-3-yl)-2-cyanopentanedioate

### Step A: 5-bromo-3-iodo-1-methyl-1H-pyrazolo[3,4-b]pyridine

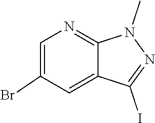

To a solution of 5-bromo-3-iodo-1H-pyrazolo[3,4-b]pyridine (7 g, 21.6 mmol, 1.0 eq.) in DMF (50 mL) was added K₂CO₃ (9 g, 64.8 mmol, 3.0 eq.) and stirred for 30 mins at 60° C. Then the Mel (2.70 mL, 43.2 mmol, 2.0 eq.) was added and the mixture was stirred at 60° C. for 1 hour. The resulting solution was diluted with 100 mL of water and extracted with EtOAc (100 mL×3). The combined organic layers were washed with brine (100 mL), dried over Na₂SO₄ and concentrated under reduced pressure. The residue was purified by flash column chromatography on silica gel (100-200 mesh silica gel, 0-13% EtOAc in PE) to give 5-bromo-3-iodo-1-methyl-1H-pyrazolo[3,4-b]pyridine (4.6 g, 63% yield) as a white solid.

LC-MS (ESI): mass calcd. for C₇H₅BrIN₃, 336.87; m/z found, 337.8 [M+H]f.

### Step B: tert-butyl 2-(5-bromo-1-methyl-1H-pyrazolo[3,4-b]pyridin-3-yl)-2-cyanoacetate

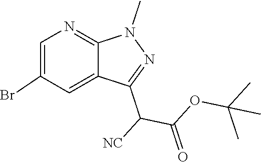 [SMILES: Cn1nc(C(C#N)C(=O)OC(C)(C)C)c2cc(Br)cnc21]

To a solution of 5-bromo-3-iodo-1-methyl-1H-pyrazolo[3,4-b]pyridine (4.6 g, 13.6 mmol, 1.0 eq.), CuI (260 mg, 1.4 mmol, 0.1 eq.), Cs₂CO₃ (13.3 g, 40.8 mmol, 3.0 eq.) and 1,2-cyclohexanediamine, (lr,2r)- (390 mg, 3.4 mmol, 0.25 eq.) in 1,4-Dioxane (80 mL) was added tert-butyl 2-cyanoacetate (3.5 g, 24.5 mmol, 1.8 eq.) and stirred at 100° C. for 6 hours. The resulting solution was diluted with 200 mL of water and extracted with EtOAc (100 mL×3). The combined organic layers were washed with brine (200 mL), dried over Na₂SO₄ and concentrated under reduced pressure. The residue was purified by flash column chromatography on silica gel (100-200 mesh silica gel, 0-13% EtOAc in PE, 5% DCM) to give tert-butyl 2-(5-bromo-1-methyl-1H-pyrazolo[3,4-b]pyridin-3-yl)-2-cyanoacetate (2 g, 42% yield) as a yellow solid. LC-MS (ESI): mass calcd. for C₁₄H₁₅BrN₄O₂, 350.04; m/z found, 351.0 [M+H]⁺.

### Step C: 1-(tert-butyl) 5-ethyl 2-(5-bromo-1-methyl-1H-pyrazolo[3,4-b]pyridin-3-yl)-2-cyanopentanedioate

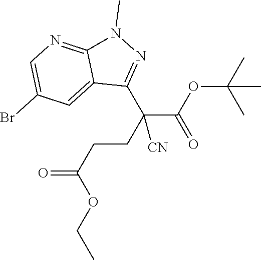 [SMILES: CCOC(=O)CCC(C#N)(C(=O)OC(C)(C)C)c1nn(C)c2ncc(Br)cc12]

To a solution of tert-butyl 2-(5-bromo-1-methyl-1H-pyrazolo[3,4-b]pyridin-3-yl)-2-cyanoacetate (700 mg, 2.0 mmol, 1.0 eq.), K₂CO₃ (551 mg, 4.0 mmol, 2.0 eq.) and benzyl(triethyl)ammonium chloride (91 mg, 399 μmol, 0.2 eq.) in ACN (15 mL) was stirred at 88° C. for 30 mins. The ethyl 3-bromopropanoate (1.28 mL, 10.0 mmol, 5.0 eq.) was added and stirred at 88° C. for 16 hours. The reaction mixture was slowly poured into water (100 mL) and extracted with EtOAc (50 mL×3). The combined organic layers were washed with brine (100 mL×3), dried over Na₂SO₄ and concentrated under reduced pressure. The residue was purified by flash column chromatography on silica gel (100-200 mesh silica gel, 0-5% EtOAc in PE) to give 1-(tert-butyl) 5-ethyl 2-(5-bromo-1-methyl-1H-pyrazolo[3,4-b]pyridin-3-yl)-2-cyanopentanedioate (300 mg, 33% yield) as a yellow solid. ¹H NMR (400 MHz, DMSO-d₆) δ 8.75 (d, J=2.0 Hz, 1H), 8.51 (d, J=2.1 Hz, 1H), 4.09 (s, 3H), 4.01 (q, J=7.1 Hz, 2H), 2.82-2.74 (m, 2H), 2.49-2.39 (m, 2H), 1.41 (s, 9H), 1.16 (t, J=7.1 Hz, 3H).

### Step D: 1-(tert-butyl) 5-ethyl 2-(5-bromo-1-methyl-1H-pyrazolo[3,4-b]pyridin-3-yl)-2-cyanopentanedioate

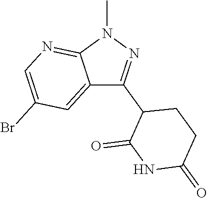 [SMILES: Cn1nc(C2CCC(=O)NC2=O)c2cc(Br)cnc21]

To a solution of 1-(tert-butyl) 5-ethyl 2-(5-bromo-1-methyl-1H-pyrazolo[3,4-b]pyridin-3-yl)-2-cyanopentanedioate (300 mg, 665 μmol,1.0 eq.) in HOAc (20 mL) and H₂SO₄ (0.5 mL) was stirred at 110° C. under N₂ and stirred at room temperature for 2 hours. The reaction mixture was slowly poured into ice water (100 mL) and extracted with EtOAc (50 mL×3). The combined organic layers were washed with brine (50 mL×3), dried over Na₂SO₄ and concentrated under reduced pressure. The residue was purified by flash column chromatography on silica gel (100-200 mesh silica gel, 0-10% EtOAc in PE) to give 3-(5-bromo-1-methyl-1H-pyrazolo[3,4-b]pyridin-3-yl)piperidine-2,6-dione (200 mg, 93% yield) as a white solid. ¹H NMR (400 MHz, DMSO-d₆) δ 11.01 (s, 1H), 8.69 (s, 1H), 8.63 (s, 1H), 4.46 (dd, J=11.0, 4.8 Hz, 1H), 4.08 (s, 3H), 2.79-2.72 (m, 1H), 2.69-2.64 (m, 1H), 2.55-2.47 (m, 1H), 2.25-2.19 (m, 1H).

### Intermediate 78. 3-(5-(4-aminobuta-1,2-dien-1-yl)benzofuran-3-yl)piperidine-2,6-dione

### Step A: 3-(5-(hydroxymethyl benzofuran-3-yl)piperidine-2,6-dione

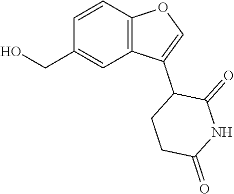 [SMILES: O=C1CCC(c2coc3ccc(CO)cc23)C(=O)N1]

3-(5-bromobenzofuran-3-yl)piperidine-2,6-dione (2.0 g, 6.49 mmol, 1.0 eq.), PdCl₂(dppf) (475 mg, 649 μmol, 0.1 eq.), (tributylstannyl)methanol (2.5 g, 7.8 mmol, 1.2 eq.) in 1,4-Dioxane (30 mL) at room temperature. The mixture was stirred at 100° C. for 16 hours. The resulting solution was diluted with 40 ml of water and extracted with DCM (30 mL×3). The combined organic layers were washed with brine (30 mL×3), dried over Na₂SO₄ and concentrated under reduced pressure. The residue was purified by silica gel chromatography (100-200 mesh silica gel, 0-10% MeOH in DCM) to give 3-(5-(hydroxymethyl)benzofuran-3-yl)piperidine-2,6-dione (400 mg, yield 24%) as a yellow solid. LC-MS (ESI): mass calcd. for C₁₄H₁₃NO₄, 259.1; m/z found, 260.3 [M+H]⁺.

### Step B: 3-(2,6-dioxopiperidin-3-yl)benzofuran-5-carbaldehyde

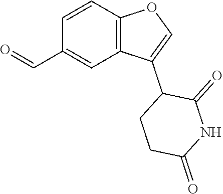 [SMILES: O=Cc1ccc2occ(C3CCC(=O)NC3=O)c2c1]

To a solution of 3-(5-(hydroxymethyl)benzofuran-3-yl)piperidine-2,6-dione (350 mg, 1.3 mmol, 1.0 eq.) in DCM (7 mL) was added Dess-Martin periodinane (687 mg, 1.6 mmol, 1.2 eq.), the mixture was stirred for 0° C. at 2 hours. The resulting solution was diluted with 20 ml of water and extracted with DCM (20 mL×3). The combined organic layers were washed with brine (20 mL×3), dried over Na₂SO₄ and concentrated under reduced pressure. The residue was purified by silica gel chromatography (100-200 mesh silica gel, 0-10% MeOH in DCM) to give 3-(2,6-dioxopiperidin-3-yl)benzofuran-5-carbaldehyde (250 mg, yield 72.0%) as a solid. LC-MS (ESI): mass calcd. for C₁₄H₁₁NO₄, 257.1; m/z found, 258.3 [M+H]⁺.

### Step C: 3-(5-(4-hydroxybuta-1,2-dien-1-yl)benzofuran-3-yl)piperidine-2,6-dione

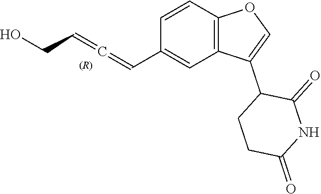

To a dried Schlenk flask were sequentially added 3-(2,6-dioxopiperidin-3-yl)benzofuran-5-carbaldehyde (200 mg, 777 μmol, 1.0 eq.), prop-2-yn-1-ol (44 mg, 777 μmol, 1.0 eq.), (S)-(+)-alpha,alpha-Diphenyl-2-pyrrolidinemethanol (130 mg, 513 μmol, 0.66 eq.), 1,4-Dioxane (5 mL) and Cupric bromide (26 mg, 117 μmol, 0.15 eq.) under the nitrogen atmosphere. The mixture was stirred at 130° C. for 5 hours. The resulting solution was diluted with 20 ml of water and extracted with EtOAc (20 mL×3). The combined organic layers were washed with brine (20 mL×3), dried over Na₂SO₄ and concentrated under reduced pressure. The residue was purified by silica gel chromatography (100-200 mesh silica gel, 0-10% MeOH in DCM) to give 3-(5-(4-hydroxybuta-1,2-dien-1-yl)benzofuran-3-yl)piperidine-2,6-dione (80.0 mg, yield 35%) as a yellow oil. LC-MS (ESI): mass calcd. for C₁₇H₁₅NO₄, 297.1; m/z found, 298.3 [M+H]⁺.

### Step D: 4-(3-(2,6-dioxopiperidin-3-yl)benzofuran-5-yl)buta-2,3-dien-1-yl methanesulfonate

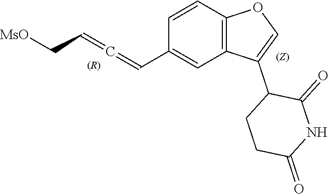

To a solution of 3-(5-(4-hydroxybuta-1,2-dien-1-yl)benzofuran-3-yl)piperidine-2,6-dione (40.0 mg, 135 μmol, 1.0 eq.) in DCM (5 mL) was added TEA (0.2 mL) and MsCl (0.1 mL), the mixture was stirred for 25° C. at 2 hours. The resulting solution was diluted with 20 ml of water and extracted with DCM (20 mL×3). The combined organic layers were washed with brine (20 mL×3), dried over Na₂SO₄ and concentrated under reduced pressure. The residue was purified by silica gel chromatography (100-200 mesh silica gel, 0-10% MeOH in DCM) to give 4-(3-(2,6-dioxopiperidin-3-yl)benzofuran-5-yl)buta-2,3-dien-1-yl methanesulfonate (20.0 mg, yield 39.6%) as a solid. LC-MS (ESI): mass calcd. for C₁₈H₁₇NO₆S, 375.0; m/z found, 376.0 [M+H]⁺.

### Step E: 3-(5-(4-azidobuta-1,2-dien-1-yl)benzofuran-3-yl)piperidine-2,6-dione

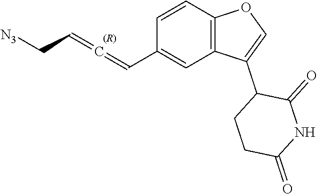

To a solution of 4-(3-(2,6-dioxopiperidin-3-yl)benzofuran-5-yl)buta-2,3-dien-1-yl methanesulfonate (50 mg, 133 μmol, 1.0 eq.) in DMF (4 mL) was added sodium azide (9 mg, 133 μmol, 1.0 eq.), the mixture was stirred for 20° C. at 2 hours. The resulting solution was diluted with 20 ml of water and extracted with EtOAc (10 mL×3). The combined organic layers were washed with brine (10 mL×3), dried over Na₂SO₄ and concentrated under reduced pressure. The residue was purified by silica gel chromatography (100-200 mesh silica gel, 0-10% MeOH in DCM) to give 3-(5-(4-azidobuta-1,2-dien-1-yl)benzofuran-3-yl)piperidine-2,6-dione (35 mg, yield 81%) as a solid. LC-MS (ESI): mass calcd. for C₁₇H₁₄N₄O₃, 322.1; m/z found, 323.0 [M+H]⁺.

### Step F: tert-butyl (4-(3-(2,6-dioxopiperidin-3-yl)benzofuran-5-yl)buta-2,3-dien-1-yl)carbamate

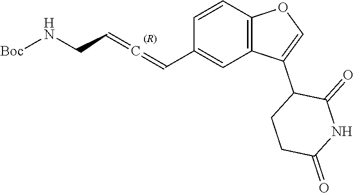

To a solution of 3-(5-(4-azidobuta-1,2-dien-1-yl)benzofuran-3-yl)piperidine-2,6-dione (40.0 mg, 124 μmol, 1.0 eq.) in THF (2 mL) and Water (2 mL) was added Triphenylphosphine (65.1 mg, 248 μmol, 2.0 eq.) and Boc₂O(54.0 mg, 248 μmol, 2.0 eq.), the mixture was stirred for 20° C. at 16 hours. The resulting solution was diluted with 20 ml of water and extracted with DCM (10 mL×3). The combined organic layers were washed with brine (10 mL×3), dried over Na₂SO₄ and concentrated under reduced pressure. The residue was purified by silica gel chromatography (100-200 mesh silica gel, 0-10% MeOH in DCM) to give tert-butyl (4-(3-(2,6-dioxopiperidin-3-yl)benzofuran-5-yl)buta-2,3-dien-1-yl)carbamate (20.0 mg, yield 40.7%) as a solid. LC-MS (ESI): mass calcd. for C₂₂H₂₄N₂O₅, 396.1; m/z found, 397.3 [M+H]⁺.

### Step G: 3-(5-(4-aminobuta-1,2-dien-1-yl)benzofuran-3-yl)piperidine-2,6-dione

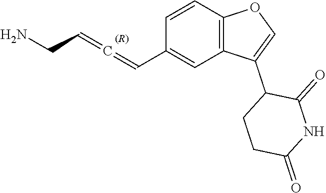

A solution of tert-butyl (4-(3-(2,6-dioxopiperidin-3-yl)benzofuran-5-yl)buta-2,3-dien-1-yl)carbamate (20 mg, 50.4 μmol, 1.0 eq.), TFA (0.8 mL) in DCM (4 mL) with stirring at room temperature. The mixture was stirred at 25° C. for 1 hour. The residue was concentrated under reduced pressure to give 3-(5-(4-aminobuta-1,2-dien-1-yl)benzofuran-3-yl)piperidine-2,6-dione (20 mg, crude) as an oil. LC-MS (ESI): mass calcd. for C₁₇H₁₆N₂O₃, 296.1; m/z found, 297.3 [M+H]⁺.

### Intermediate 79. 3-(5-(3-aminoprop-1-yn-1-yl-3,3-d2)benzofuran-3-yl)piperidine-2,6-dione

### Step A: 3-(trimethylsilyl)prop-2-yn-1,1-d2-1-ol

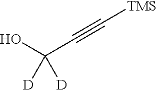

In a 100 ml single-necked bottle, add ethyl 3-(trimethylsilyl)propiolate (3.0 g, 17.6 mmol, 1.0 eq.) and THF (45 mL), cool down to 0° C., add Lithium tetrahydrido(2H₄)aluminate(1-) (740 mg, 17.6 mmol, 1.0 eq.) in batches, after the addition is complete, keep the reaction for 1 hour. An appropriate amount of water was slowly added dropwise to the reaction solution to quench the reaction. After the drop was completed, an appropriate amount of anhydrous sodium sulfate was added to dry, filtered, and the filtrate was concentrated to obtain 3-(trimethylsilyl)prop-2-yn-1,1-d2-1-ol (1.50 g, crude) as a colorless liquid.

### Step B: tert-butyl (diethoxyphosphoryl)(3-(trimethylsilyl)prop-2-yn-1-yl-1,1-d2)carbamate

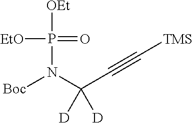 [SMILES: [2H]C([2H])(C#C[Si](C)(C)C)N(C(=O)OC(C)(C)C)P(=O)(OCC)OCC]

3-(trimethylsilyl)prop-2-yn-1,1-d2-1-ol (300 mg, 2.3 mmol, 1.0 eq.), tert-butyl (diethoxyphosphoryl)carbamate (583 mg, 2.3 mmol, 1.0 eq.) and Triphenylphosphine (604 mg, 2.3 mmol, 1.0 eq.) were dissolved in anhydrous THF (15 mL). The reaction mixture was flushed with argon and subsequently cooled to 0° C. DIAD (466 mg, 2.3 mmol, 1.0 eq.) was added dropwise to the reaction over 10 minutes. Cooling was removed and the reaction mixture was stirred at room temperature for 18 hours. The solvent was removed in vacuo and the crude reaction mixture containing intermediate tert-butyl (diethoxyphosphoryl)(3-(trimethylsilyl)prop-2-yn-1-yl-1,1-d2)carbamate (900 mg, crude) was directly submitted to the next step.

### Step C: 3-(trimethylsilyl)prop-2-yn-1,1-d2-1-amine

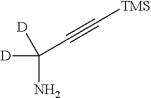

tert-butyl (diethoxyphosphoryl)(3-(trimethylsilyl)prop-2-yn-1-yl-1,1-d2)carbamate (800 mg, 1.1 mmol, 1.0 eq.) was dissolved in HCl/dioxane (10 mL). Then the reaction mixture was sealed and left to stir overnight. The reaction mixture was filtered, the residue was triturated with diethyl ether and dried in vacuo to give 3-(trimethylsilyl)prop-2-yn-1,1-d2-1-amine (100 mg, crude) as a white solid.

### Step D: tert-butyl (3-(trimethylsilyl)prop-2-yn-1-yl-1,1-d2)carbamate

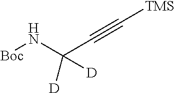 [SMILES: [2H]C([2H])(C#C[Si](C)(C)C)NC(=O)OC(C)(C)C]

3-(trimethylsilyl)prop-2-yn-1,1-d2-1-amine (200 mg, 1.5 mmol, 1.0 eq.) were added DCM (6 mL) and TEA (0.7 mL). The suspension was stirred at room temperature for 10 minutes prior to addition of di-tert-butyl dicarbonate (405 mg, 1.86 mmol, 1.2 eq.) in DCM (2 mL). The clear solution was stirred for 2 hours and concentrated by rotary evaporation to give a white solid. The material was treated with ethyl acetate, water and iN aq. KHSO₄. The organic layer was extracted with saturated aq. NaHCO₃ solution, dried over Na₂SO₄, filtered and concentrated to give tert-butyl (3-(trimethylsilyl)prop-2-yn-1-yl-1,1-d2)carbamate (300 mg, crude) as a colorless oil. The material was used in the next step without further purification.

### Step E: tert-butyl (prop-2-yn-1-yl-1,1-d2)carbamate

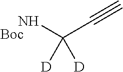

Crude tert-butyl (3-(trimethylsilyl)prop-2-yn-1-yl-1,1-d2)carbamate (300 mg, 1.3 mmol, 1.0 eq.) was dissolved in MeOH (6 mL), to which K₂CO₃ (904 mg, 6.5 mmol, 5.0 eq.) was added. The suspension was stirred at room temperature for 2 hours. The reaction mixture was diluted with ethyl acetate and water and layers were separated. The organic layer was extracted with water and brine, dried over Na₂SO₄. The residue was applied on a silica gel chromatography and eluted with EA/PE (⅕) to give tert-butyl (prop-2-yn-1-yl-1,1-d2)carbamate (160 mg, yield 78%) as an oil. ¹H NMR (400 MHz, CDCl₃) δ 4.68 (s, 1H), 2.21 (s, 1H), 1.46 (s, 9H).

### Step F: tert-butyl (3-(3-(2,6-dioxopiperidin-3-yl)benzofuran-5-yl)prop-2-yn-1-yl-1,1-d2)carbamate

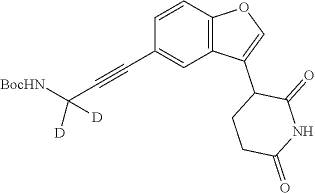 [SMILES: [2H]C([2H])(C#Cc1ccc2occ(C3CCC(=O)NC3=O)c2c1)NC(=O)OC(C)(C)C]

To a solution of 3-(5-bromobenzofuran-3-yl)piperidine-2,6-dione (50.0 mg, 162 μmol, 1.0 eq.) in DMF (3 mL) was added tert-butyl (prop-2-yn-1-yl-1,1-d2)carbamate (30.0 mg, 191 μmol, 1.2 eq.), Bis-(triphenylphosphino)-palladous chloride (11 mg, 16.2 μmol, 0.1 eq.), CuI (3.1 mg, 16.2 μmol, 0.1 eq.), TEA (0.3 mL) at room tempreature. The mixture was then stirred at 80° C. for 6 hours under N₂. The resulting solution was diluted with 10 mL of water, then extracted with EtOAc (3×10 mL) and washed with brine (10 mL). The combined organic layers were washed with brine, dried over anhydrous Na₂SO₄ and concentrated under reduced pressure. The residue was purified by flash column chromatography on silica gel (100-200 mesh silica gel, 0-60% EtOAc in PE) to afford tert-butyl (3-(3-(2,6-dioxopiperidin-3-yl)benzofuran-5-yl)prop-2-yn-1-yl-1,1-d2)carbamate (30.0 mg, yield 48%) as a yellow solid. LC-MS (ESI): mass calcd. for C₂₁H₂₀D₂N₂O₅, 384.1; m/z found, 385.3 [M+H]⁺.

### Step G: 3-(5-(3-aminoprop-1-yn-1-yl-3,3-d2)benzofuran-3-yl)piperidine-2,6-dione

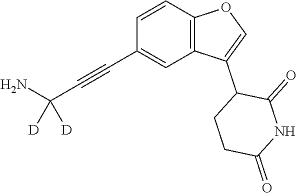 [SMILES: [2H]C([2H])(N)C#Cc1ccc2occ(C3CCC(=O)NC3=O)c2c1]

A solution of tert-butyl (3-(3-(2,6-dioxopiperidin-3-yl)benzofuran-5-yl)prop-2-yn-1-yl-1,1-d2)carbamate (25.0 mg, 65.0 μmol, 1.0 eq.), TFA (0.4 mL) in DCM (2 mL) with stirring at room temperature. The mixture was stirred at 25° C. for 1 hour. The residue was concentrated under reduced pressure to give 3-(5-(3-aminoprop-1-yn-1-yl-3,3-d2)benzofuran-3-yl)piperidine-2,6-dione (20 mg, crude) as an oil. LC-MS (ESI): mass calcd. for C₁₆H₁₂D₂N₂O₃, 284.1; m/z found, 285.3 [M+H]⁺.

### Intermediate 80. 6-chloro-4-(1,1-dioxidotetrahydro-2H-thiopyran-4-yl)-1,3-dimethyl-1,3-dihydro-2H-imidazo[4,5-c]pyridin-2-one

### Step A: 6-chloro-4-(3,6-dihydro-2H-thiopyran-4-yl)-1,3-dimethyl-1,3-dihydro-2H-imidazo[4,5-c]pyridin-2-one

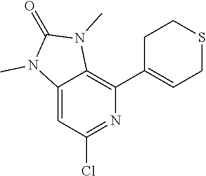 [SMILES: Cn1c(=O)n(C)c2c(C3=CCSCC3)nc(Cl)cc21]

To a solution of 2-(3,6-dihydro-2H-thiopyran-4-yl)-4,4,5,5-tetramethyl-1,3,2-dioxaborolane (2.0 g, 8.8 mmol, 1.2 eq.), 4,6-dichloro-1,3-dimethyl-1,3-dihydro-2H-imidazo[4,5-c]pyridin-2-one (1.7 g, 7.4 mmol, 1.0 eq.) and K₂CO₃ (3.1 g, 22.1 mmol, 3.0 eq.) in 1,4-Dioxane (20 mL): H₂O(2 mL) was added Pd(dppf)Cl₂ (539 mg, 737 μmol, 0.1 eq.) at r.t. The mixture was stirred at 100° C. for 6 hours under N₂ atmosphere. The resulting solution was diluted with 100 ml of water, then extracted with EtOAc (3*20 mL) and washed with brine (2*20 mL). The combined organic layer was dried over anhydrous sodium sulfate and concentrated under reduced pressure. The residue was purified by flash column chromatography on silica gel (100-200 mesh silica gel, 0-50% EtOAc in PE) to afford the product6-chloro-4-(3,6-dihydro-2H-thiopyran-4-yl)-1,3-dimethyl-1,3-dihydro-2H-imidazo[4,5-c]pyridin-2-one (700 mg, 32.1% yield) as a yellow solid. LC-MS (ESI): mass calcd. for C₁₃H₁₄ClN₃OS, 295.05; m/z found, 296 [M+H]⁺.

### Step B: 6-chloro-4-(1,1-dioxido-3,6-dihydro-2H-thiopyran-4-yl)-1,3-dimethyl-1,3-dihydro-2H-imidazo[4,5-c]pyridin-2-one

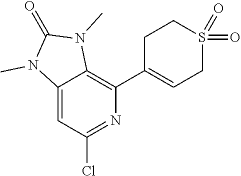 [SMILES: Cn1c(=O)n(C)c2c(C3=CCS(=O)(=O)CC3)nc(Cl)cc21]

To a solution of 6-chloro-4-(3,6-dihydro-2H-thiopyran-4-yl)-1,3-dimethyl-1,3-dihydro-2H-imidazo[4,5-c]pyridin-2-one (900 mg, 3.0 mmol, 1.0 eq.) in DCM (20 mL) was added Oxone (18.7 g, 30.4 mmol, 10.0 eq.) at room temperature. The mixture was stirred at 40° C. for 36 hours under N₂ atmosphere. The resulting solution was diluted with 100 ml of water, then extracted with DCM (3*20 mL) and washed with brine (2*20 mL). The combined organic layers were dried over anhydrous sodium sulfate and concentrated under reduced pressure. The residue was purified by flash column chromatography on silica gel (100-200 mesh silica gel, 0-30% EtOAc in PE) to afford 6-chloro-4-(1,1-dioxido-3,6-dihydro-2H-thiopyran-4-yl)-1,3-dimethyl-1,3-dihydro-2H-imidazo[4,5-c]pyridin-2-one (770 mg, 77.2% yield) as a yellow solid. LC-MS (ESI): mass calcd. for C₁₃H₁₄ClN₃O₃S, 327.04; m/z found, 328 [M+H]⁺.

### Step C: 6-chloro-4-(1,1-dioxidotetrahydro-2H-thiopyran-4-yl)-1,3-dimethyl-1,3-dihydro-2H-imidazo[4,5-c]pyridin-2-one

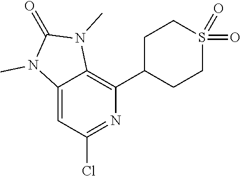 [SMILES: Cn1c(=O)n(C)c2c(C3CCS(=O)(=O)CC3)nc(Cl)cc21]

To a solution of 6-chloro-4-(1,1-dioxido-3,6-dihydro-2H-thiopyran-4-yl)-1,3-dimethyl-1,3-dihydro-2H-imidazo[4,5-c]pyridin-2-one (300 mg, 915 μmol, 1.0 eq.), Pd/C (120 mg, w.t.=10%) in MeOH (3 mL) and THF (1 mL) was stirred at 50° C. for 24 hours under H₂ atmosphere. The resulting solution was removed under reduced pressure. The residue was purified by Prep-HPLC to give 6-chloro-4-(1,1-dioxidotetrahydro-2H-thiopyran-4-yl)-1,3-dimethyl-1,3-dihydro-2H-imidazo[4,5-c]pyridin-2-one (20 mg, 6.6% yield) as a white solid.

LC-MS (ESI): mass calcd. for C₁₃H₁₆ClN₃O₃S, 329.06; m/z found, 330 [M+H]⁺.

### Intermediate 81. tert-butyl (2-chloro-4-(dimethylamino)pyrimidin-5-yl)carbamate

### Step A: 2-chloro-N,N-dimethyl-5-nitropyrimidin-4-amine

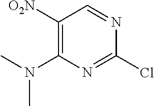

To a solution of 2,4-dichloro-5-nitropyrimidine (7.5 g, 38.7 mmol, 1.0 eq.) in THE (80 mL) was added K₂CO₃ (10.7 g, 77.3 mmol, 2.0 eq.) and dimethylamine (21 mL, 42.5 mmol, 2 M in THF, 1.1 eq.). The solution was stirred at 25° C. for 1 hour to give yellow solution. LCMS showed the reaction was completed. The resulting solution was diluted with 500 mL of water and extracted with EtOAc (3×300 mL). The combined organic layers were washed with brine (500 mL), dried over Na₂SO₄, and concentrated under reduced pressure. The residue was purified by flash chromatography (100-200 mesh silica gel, 0% ˜30% EtOAC in PE) to afford 2-chloro-N,N-dimethyl-5-nitropyrimidin-4-amine (6.6 g, yield 80%) as a yellow solid. LC-MS (ESI): mass calcd. for C₆H₇ClN₄O₂, 202.0; m/z found, 203.0 [M+H]⁺.

### Step B: 2-chloro-N₄,N₄-dimethylpyrimidine-4,5-diamine

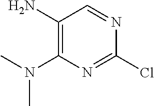

To a solution of 2-chloro-N,N-dimethyl-5-nitropyrimidin-4-amine (6.6 g, 32.6 mmol, 1.0 eq.) in EtOH (50 mL) and Water (50 mL)was added NH₄Cl (17.4 g, 326 mmol, 10.0 eq.) and iron (18.2 g, 326 mmol, 10.0 eq.).The solution was stirred at 80° C. for 2 hours to give black solution. LCMS showed the reaction was completed. The resulting solution was diluted with 500 mL of water and extracted with EtOAc (3×300 mL). The combined organic layers were washed with brine (500 mL), dried over Na₂SO₄, and concentrated under reduced pressure. The residue was purified by flash chromatography (100-200 mesh silica gel, 0% ˜50% EtOAC in PE) to afford 2-chloro-N₄,N₄-dimethylpyrimidine-4,5-diamine (2.9 g, yield 49%) as a yellow solid. LC-MS (ESI): mass calcd. for C₆H₉ClN₄, 172.1; m/z found, 173.1 [M+H]⁺.

### Step C: tert-butyl (2-chloro-4-(dimethylamino)pyrimidin-5-yl)carbamate

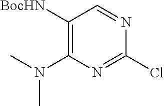 [SMILES: CN(C)c1nc(Cl)ncc1NC(=O)OC(C)(C)C]

To a solution of 2-chloro-N4,N4-dimethylpyrimidine-4,5-diamine (500 mg, 2.90 mmol, 1.0 eq.) in THF (10 mL) was added KHMDS (4.4 mL, 4.3 mmol, 1 M in THF, 1.5 eq.), the solution was stirred at 0° C. for 30 mins, then added Boc₂O(732 μL, 3.2 mmol, 1.1 eq.) was stirred at 25° C. for 1 hour to give yellow solution. LCMS showed the reaction was completed. The solution was quenched sat. NH₄Cl solution, extracted with EtOAC (100 mL) and the layers were separated. The organic layer was washed with brine (100 mL), dried over Na₂SO₄, filtered and concentrated. The residue was purified by flash chromatography (100-200 mesh silica gel, 0% ˜50% EtOAC in PE) to afford tert-butyl (2-chloro-4-(dimethylamino)pyrimidin-5-yl)carbamate (350 mg, yield 42%) as a yellow solid. LC-MS (ESI): mass calcd. for C₁₁H₁₇ClN₄O₂, 272.3; m/z found, 273.3 [M+H]⁺.

### Intermediate 82. 6-(1-(6-chloropyridin-3-yl)-2,2-difluorovinyl)-4-isopropyl-1,3-dimethyl-1,3-dihydro-2H-benzo[d]imidazol-2-one

### Step A: 2,2-difluoro-1-(tributylstannyl)vinyl 4-methylbenzenesulfonate

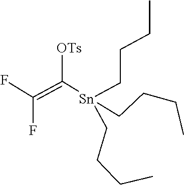 [SMILES: CCC[CH2][Sn]([CH2]CCC)([CH2]CCC)[C](OS(=O)(=O)c1ccc(C)cc1)=C(F)F]

To a solution of 2,2,2-trifluoroethyl 4-methylbenzenesulfonate (3 g, 11.8 mmol, 1.0 eq.) in THF (30 mL) was added LDA (12 mL, 24.0 mmol, 2.0 eq., 2M in THF) at −65° C. under N₂ atmosphere. The reaction was stirred at −65° C. for 30 mins, added n-Bu₃SnCl (3.2 mL, 11.8 mmol, 1.0 eq.) dropwise at −65° C. The reaction was warmed to 25° C. and stirred for 2 hours. The resulting solution was diluted with 50 ml of water and extracted with EtOAc (3×50 mL). The combined organic layers were washed with brine, dried over anhydrous Na₂SO₄ and concentrated under reduced pressure. The residue was purified by flash column chromatography on silica gel (100-200 mesh silica gel, 0-35% EtOAc in PE) to afford 2,2-difluoro-1-(tributylstannyl)vinyl 4-methylbenzenesulfonate (3.4 g, 55% yield) as a colorless oil. No MS signal. ¹H NMR (400 MHz, CDCl₃) δ 7.78 (d, J=8.3 Hz, 2H), 7.34 (d, J=8.1 Hz, 2H), 2.46 (s, 3H), 1.67-1.59 (m, 2H), 1.53 (dd, J=7.3, 3.0 Hz, 4H), 1.37-1.32 (m, 6H), 1.18-1.06 (m, 6H), 0.94-0.90 (m, 10H)

### Step B: 1-(6-chloropyridin-3-yl)-2,2-difluorovinyl 4-methylbenzenesulfonate

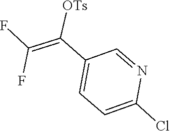 [SMILES: Cc1ccc(S(=O)(=O)OC(=C(F)F)c2ccc(Cl)nc2)cc1]

To a solution of 2-chloro-5-iodopyridine (500 mg, 2.1 mmol, 1.2 eq.) in DMF (10mL) was added 2,2-difluoro-1-(tributylstannyl)vinyl 4-methylbenzenesulfonate (910 mg, 1.7 mmol, 1.0 eq.), Pd(PPh₃)₄ (201 mg, 174 μmol, 0.1 eq.) and CuI (33 mg, 174 μmol, 0.1 eq.) at 25° C. The reaction was stirred at 80° C. for 16 hours under N₂ atmosphere. The resulting solution was diluted with 50 ml of water and extracted with EtOAc (3×50 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford 1-(6-chloropyridin-3-yl)-2,2-difluorovinyl 4-methylbenzenesulfonate (550 mg, 92% yield) as a yellow solid. LC-MS (ESI): mass calcd. for C₁₄H₁₀ClF₂NO₃S, 345.0; m/z found, 346.0 [M+H]⁺.

### Step C: 6-(1-(6-chloropyridin-3-yl)-2,2-difluorovinyl)-4-isopropyl-1,3-dimethyl-1,3-dihydro-2H-benzo[d]imidazol-2-one

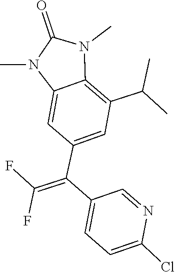 [SMILES: CC(C)c1cc(C(=C(F)F)c2ccc(Cl)nc2)cc2c1n(C)c(=O)n2C]

To a solution of 1-(6-chloropyridin-3-yl)-2,2-difluorovinyl 4-methylbenzenesulfonate (300 mg, 868 μmol, 1.0 eq.) in 1,4-Dioxane: Water (15 mL: 5 mL) was added (7-isopropyl-1,3-dimethyl-2-oxo-2,3-dihydro-1H-benzo[d]imidazol-5-yl)boronic acid (237 mg, 954 μmol, 1.1 eq.), Pd(PPh₃)₄ (90 mg, 86.8 μmol, 0.1 eq.), K₃PO₄ (313 mg, 1.5 mmol, 1.7 eq.) and PCy₃ (97 mg, 347 μmol, 0.4 eq.). The reaction was stirred at 100° C. for 3 hours under N₂ atmosphere. The resulting solution was diluted with 50 mL of water and extracted with EtOAc (3×50 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford 6-(1-(6-chloropyridin-3-yl)-2,2-difluorovinyl)-4-isopropyl-1,3-dimethyl-1,3-dihydro-2H-benzo[d]imidazol-2-one (140 mg, 43% yield) as a yellow solid. LC-MS (ESI): mass calcd. for C₁₉H₁₈ClF₂N₃O, 377.1; m/z found, 378.1 [M+H]⁺.

### Intermediate 83. 7-bromo-4-(7-isopropyl-1,3-dimethyl-2-oxo-2,3-dihydro-1H-benzo[d]imidazol-5-yl)-3,4-dihydro-2H-benzo[b][1,4]oxazine-6-carbonitrile

### Step A: 4-(7-isopropyl-1,3-dimethyl-2-oxo-2,3-dihydro-1H-benzo[d]imidazol-5-yl)-3,4-dihydro-2H-benzo[b][1,4]oxazine-6-carbonitrile

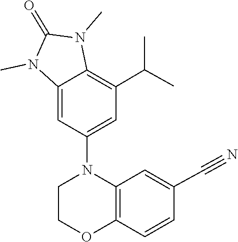 [SMILES: CC(C)c1cc(N2CCOc3ccc(C#N)cc32)cc2c1n(C)c(=O)n2C]

To a solution of 3,4-dihydro-2H-benzo[b][1,4]oxazine-6-carbonitrile (100 mg, 624 μmol, 1 eq.) and 6-bromo-4-isopropyl-1,3-dimethyl-1,3-dihydro-2H-benzo[d]imidazol-2-one (212 mg, 749 μmol, 1.2 eq,) in 1,4-Dioxane (3 mL) were added 2-Dicyclohexylphosphino-2′,6′-diisopropoxybiphenyl (58 mg, 125 μmol, 0.2 eq.), Ru-Phos Pd G1 (91 mg, 125 μmol, 0.2 eq.) and Cs₂CO₃ (610 mg, 1.87 mmol, 3 eq.). The reaction mixture was stirred at 100° C. for 16 hours. The reaction mixture was poured into water and extracted with EtOAc (10 mL*3). The organic layer was washed with H₂O, dried Na₂SO₄ and concentrated to give a residue. The residue was purified by column chromatography on silica geleluted with PE/EtOAc (100:1-1:1) to give 4-(7-isopropyl-1,3-dimethyl-2-oxo-2,3-dihydro-1H-benzo[d]imidazol-5-yl)-3,4-dihydro-2H-benzo[b][1,4]oxazine-6-carbonitrile (60 mg, 26% yield) as brown oil.

LC-MS (ESI): mass calcd. for C₂₁H₂₂N₄O₂, 362.17; m/z found, 363.17 [M+H]⁺.

### Step B: 7-bromo-4-(7-isopropyl-1,3-dimethyl-2-oxo-2,3-dihydro-1H-benzo[d]imidazol-5-yl)-3,4-dihydro-2H-benzo[b][1,4]oxazine-6-carbonitrile

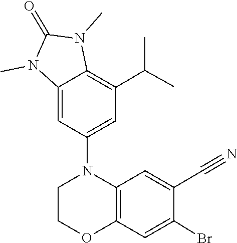 [SMILES: CC(C)c1cc(N2CCOc3cc(Br)c(C#N)cc32)cc2c1n(C)c(=O)n2C]

To a solution of 4-(7-isopropyl-1,3-dimethyl-2-oxo-2,3-dihydro-1H-benzo[d]imidazol-5-yl)-3,4-dihydro-2H-benzo[b][1,4]oxazine-6-carbonitrile (100 mg, 276 μmol, 1 eq.) in DCM (2 mL) was added NBS (52 mg, 290 μmol, 1.05 eq.). The reaction mixture was stirred at 0° C. for 2 hours. The reaction mixture was poured into water and extracted with DCM (30 ml, 10*3 mL). The organic layer was washed with brine, dried Na₂SO₄ and concentrated to give a residue. The residue was purified by column chromatography on silica geleluted with PE/EtOAc (100:1˜1:1) to give 7-bromo-4-(7-isopropyl-1,3-dimethyl-2-oxo-2,3-dihydro-1H-benzo[d]imidazol-5-yl)-3,4-dihydro-2H-benzo[b][1,4]oxazine-6-carbonitrile (80 mg, 65%) as brown solid. LC-MS (ESI): mass calcd. for C₂₁H₂₁BrN₄O₂, 440.08; m/z found, 441.08 [M+H]⁺.

### Intermediate 84. 5-(8-(1,3-dimethyl-7-morpholino-2-oxo-1,2-dihydro-1,6-naphthyridin-5-yl)-6-methyl-5-oxo-5,6-dihydro-2,6-naphthyridin-3-yl)picolinic acid

### Step A: methyl 5-(6-methyl-5-oxo-8-(4,4,5,5-tetramethyl-1,3,2-dioxaborolan-2-yl)-5,6-dihydro-2,6-naphthyridin-3-yl)picolinate

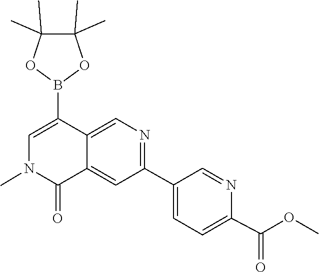

To a mixture of methyl 5-(8-bromo-6-methyl-5-oxo-5,6-dihydro-2,6-naphthyridin-3-yl)picolinate (360 mg, 962.1 μmol, 1.0 eq.), Pd(dppf)Cl₂(70 mg, 96.2 μmol, 0.1 eq.), 4,4,4′,4′,5,5,5′,5′-octamethyl-2,2′-bi(1,3,2-dioxaborolane) (293 mg, 1.2 mmol, 1.2 eq.) and Potassium acetate (283 mg, 2.9 mmol, 3.0 eq.) in 1,4-dioxane (10 mL) was stirred at 80° C. for 6 hours under N₂ atmosphere. The reaction was monitored by LCMS. The resulting solution was diluted with 10 mL of water and extracted with EtOAc (3*30 mL). The combined organic layers were washed with brine, dried over anhydrous Na₂SO₄ and concentrated under reduced pressure. The residue was purified by flash column chromatography on silica gel (100-200 mesh silica gel, 0-40% EtOAc in PE) to afford methyl 5-(6-methyl-5-oxo-8-(4,4,5,5-tetramethyl-1,3,2-dioxaborolan-2-yl)-5,6-dihydro-2,6-naphthyridin-3-yl)picolinate (200 mg, yield 49.3%) as a yellow solid. LC-MS (ESI): mass calcd. for C₂₂H₂₄BN₃O₅, 421.1; m/z found, 422.1 [M+H]⁺.

### Step B: methyl 5-(8-(7-chloro-1,3-dimethyl-2-oxo-1,2-dihydro-1,6-naphthyridin-5-yl)-6-methyl-5-oxo-5,6-dihydro-2,6-naphthyridin-3-yl)picolinate

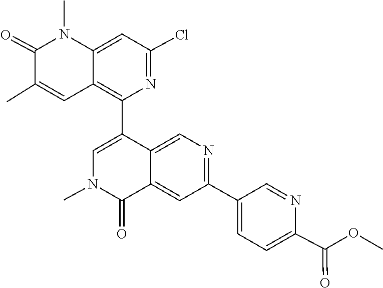 [SMILES: COC(=O)c1ccc(-c2cc3c(=O)n(C)cc(-c4nc(Cl)cc5c4cc(C)c(=O)n5C)c3cn2)cn1]

A mixture of methyl 5-(6-methyl-5-oxo-8-(4,4,5,5-tetramethyl-1,3,2-dioxaborolan-2-yl)-5,6-dihydro-2,6-naphthyridin-3-yl)picolinate (200 mg, 475 μmol, 1.0 eq.), Pd(dppf)Cl₂ (35 mg, 47.5 μmol, 0.1 eq.), Na₂CO₃ (151 mg, 1.4 mmol, 3.0 eq.) and 5,7-dichloro-1,3-dimethyl-1,6-naphthyridin-2(1H)-one (115 mg, 475 μmol, 1.0 eq.) in 1,4-dioxane (10 mL) and water (1 mL) was stirred at 80° C. for 6 hours under N₂ atmosphere. The reaction was monitored by LCMS. The resulting solution was diluted with 10 mL of water and extracted with EtOAc (3*20 mL). The combined organic layers were washed with brine, dried over anhydrous Na₂SO₄ and concentrated under reduced pressure. The residue was purified by flash column chromatography on silica gel (100-200 mesh silica gel, 50-100% EtOAc in PE) to give methyl 5-(8-(7-chloro-1,3-dimethyl-2-oxo-1,2-dihydro-1,6-naphthyridin-5-yl)-6-methyl-5-oxo-5,6-dihydro-2,6-naphthyridin-3-yl)picolinate (70 mg, yield 29.4%) as a yellow solid.

LC-MS (ESI): mass calcd. for C₂₆H₂₀ClN₅O₄, 501.1; m/z found, 502.1 [M+H]⁺.

### Step C: methyl 5-(8-(1,3-dimethyl-7-morpholino-2-oxo-1,2-dihydro-1,6-naphthyridin-5-yl)-6-methyl-5-oxo-5,6-dihydro-2,6-naphthyridin-3-yl)picolinate

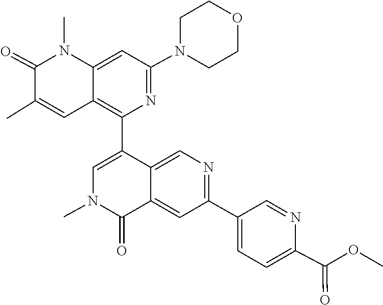 [SMILES: COC(=O)c1ccc(-c2cc3c(=O)n(C)cc(-c4nc(N5CCOCC5)cc5c4cc(C)c(=O)n5C)c3cn2)cn1]

To a mixture of methyl 5-(8-(7-chloro-1,3-dimethyl-2-oxo-1,2-dihydro-1,6-naphthyridin-5-yl)-6-methyl-5-oxo-5,6-dihydro-2,6-naphthyridin-3-yl)picolinate (70 mg, 139 μmol, 1.0 eq.), morpholine (18 mg, 209 μmol, 1.5 eq.), X-Phos (13 mg, 27.9 μmol, 0.2 eq.) and Cs₂CO₃ (136 mg, 418 μmol, 3.0 eq.) in 1,4-dioxane (10 mL) was added Pd₂(dba)₃ (13 mg, 13.9 μmol, 0.1 eq.), the mixture was stirred at 90° C. for 6 hours under N₂ atmosphere. The reaction was monitored by LCMS. The resulting solution was diluted with 10 mL of water and extracted with EtOAc (3*30 mL). The combined organic layers were washed with brine, dried over anhydrous Na₂SO₄ and concentrated under reduced pressure. The residue was purified by flash column chromatography on silica gel (100-200 mesh silica gel, 40-100% EtOAc in PE) to afford methyl 5-(8-(1,3-dimethyl-7-morpholino-2-oxo-1,2-dihydro-1,6-naphthyridin-5-yl)-6-methyl-5-oxo-5,6-dihydro-2,6-naphthyridin-3-yl)picolinate (40 mg, yield 51.9%) as a yellow solid. LC-MS (ESI): mass calcd. for C₃₀H₂₈N₆O₅, 552.2; m/z found, 553.2 [M+H]⁺.

### Step D: 5-(8-(1,3-dimethyl-7-morpholino-2-oxo-1,2-dihydro-1,6-naphthyridin-5-yl)-6-methyl-5-oxo-5,6-dihydro-2,6-naphthyridin-3-yl)picolinic acid

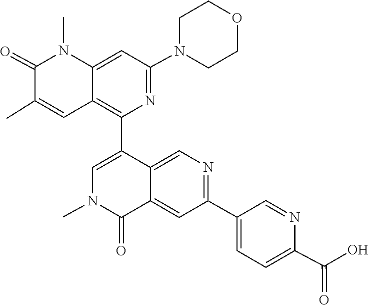 [SMILES: Cc1cc2c(-c3cn(C)c(=O)c4cc(-c5ccc(C(=O)O)nc5)ncc34)nc(N3CCOCC3)cc2n(C)c1=O]

To a solution of methyl 5-(8-(1,3-dimethyl-7-morpholino-2-oxo-1,2-dihydro-1,6-naphthyridin-5-yl)-6-methyl-5-oxo-5,6-dihydro-2,6-naphthyridin-3-yl)picolinate (20 mg, 36.2 μmol, 1.0 eq.) in THF (1 mL) and water (0.5 mL), was added LiGH (5 mg, 181 μmol, 5.0 eq.), then the mixture was stirred at 25° C. for 6 hours under N₂ atmosphere. On completion, the resulting solution was poured to ice water, adjusted the pH to 3 with HCl (aq. 2.0 N). and extracted with EtOAc (3*20 mL). The organic layer was washed with H₂O, dried Na₂SO₄ and concentrated under reduced pressure afford 5-(8-(1,3-dimethyl-7-morpholino-2-oxo-1,2-dihydro-1,6-naphthyridin-5-yl)-6-methyl-5-oxo-5,6-dihydro-2,6-naphthyridin-3-yl)picolinic acid (10 mg, yield 51.3%) as a white solid. LC-MS (ESI): mass calcd. for C₂₉H₂₆N₆O₅, 538.2; m/z found, 539.2 [M+H]⁺.

### Intermediate 85. methyl 5-(8-(1,3-dimethyl-2-oxo-7-(tetrahydro-2H-pyran-4-yl)-1,2-dihydro-1,6-naphthyridin-5-yl)isoquinolin-3-yl)picolinate

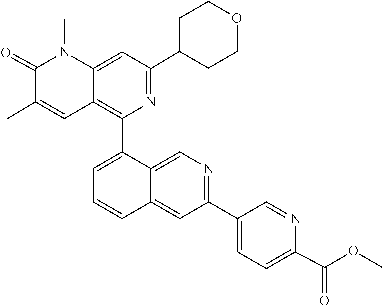 [SMILES: COC(=O)c1ccc(-c2cc3cccc(-c4nc(C5CCOCC5)cc5c4cc(C)c(=O)n5C)c3cn2)cn1]

To a solution of methyl 5-(8-(7-chloro-1,3-dimethyl-2-oxo-1,2-dihydro-1,6-naphthyridin-5-yl)isoquinolin-3-yl)picolinate (60 mg, 127 μmol, 1.0 eq.), HE (65 mg, 255 μmol, 2.0 eq.), NiBr2-d(OMe)-bpy (62 mg, 127 μmol, 1.0 eq.) in DMA (2 mL) was added 1,3-dioxoisoindolin-2-yl tetrahydro-2H-pyran-4-carboxylate was irradiated (88 mg, 319 μmol, 2.5 eq.), followed by NaHCO₃ (22 mg, 255 μmol, 2.0 eq.). The mixture was stirred under 390 nm purple LED at room temperature for 16 hours. The resulting solution was diluted with 50 mL of water and extracted with EtOAc (20 mL×3). The combined organic layers were washed with brine (50 mL), dried over Na₂SO₄ and concentrated under reduced pressure. The residue was purified by flash column chromatography on silica gel (100-200 mesh silica gel, 0-4% MeOH in DCM) to give methyl 5-(8-(1,3-dimethyl-2-oxo-7-(tetrahydro-2H-pyran-4-yl)-1,2-dihydro-1,6-naphthyridin-5-yl)isoquinolin-3-yl)picolinate (15 mg, 23% yield) as a yellow solid. LC-MS (ESI): mass calcd. for C₃₁H₂₈N₄O₄, 520.59; m/z found, 521.3 [M+H]⁺.

### Intermediate 86. 3-(5-bromo-1-methyl-11H-pyrrolo[2,3-c]pyridin-3-yl)piperidine-2,6-dione

### Step A: 5-bromo-1-methyl-1H-pyrrolo[2,3-c]pyridine

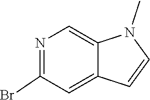

To a solution of 5-bromo-1H-pyrrolo[2,3-c]pyridine (5 g, 25.4 mmol, 1.0 eq.) and NaH (1.5 g, 38.1 mmol, 1.5 eq., w.t. =60%) in DMF (50 mL) was added Mel (3.2 mL, 50.8 mmol, 2 eq.) at 0° C. The mixture was stirred at 25° C. under N₂ for 2 hours. The resulting solution was diluted with 100 ml of water and extracted with EtOAc (60 mL×3). The combined organic layers were washed with brine, dried over Na₂SO₄ and concentrated under reduced pressure. The residue was purified with flash column chromatography on silica gel (EtOAc in PE, from 0% to 5%) to afford 5-bromo-1-methyl-1H-pyrrolo[2,3-c]pyridine (4.8 g, 89.6% yield) as a white solid. LC-MS (ESI): mass calcd. for C₈H₇BrN₂, 211; m/z found, 211/213 [M+H]⁺.

### Step B: ethyl 2-(5-bromo-1-methyl-1H-pyrrolo[2,3-c]pyridin-3-yl)-2-oxoacetate

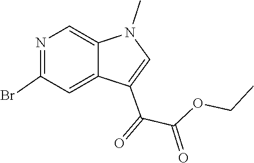 [SMILES: CCOC(=O)C(=O)c1cn(C)c2cnc(Br)cc12]

To a solution of 5-bromo-1-methyl-1H-pyrrolo[2,3-c]pyridine (1.0 g, 4.7 mmol, 1.0 eq.) and ethyl 2-chloro-2-oxoacetate (1.3 g, 9.5 mmol, 2 eq.) in DCM was added Aluminum chloride (3.2 g, 23.7 mmol, 5 eq.) at 0° C. The mixture was stirred at 20° C. under N₂ for 2 hours.

The resulting solution was diluted with 30 ml of water and extracted with EtOAc (30 mL×3). The combined organic layers were washed with brine, dried over Na₂SO₄ and concentrated under reduced pressure. The residue was purified with flash column chromatography on silica gel (EtOAc in PE, from 0% to 30%) to afford ethyl 2-(5-bromo-1-methyl-1H-pyrrolo[2,3-c]pyridin-3-yl)-2-oxoacetate (1 g, 67.8% yield) as a white solid. LC-MS (ESI): mass calcd. for C₁₂H₁₁BrN₂O₃, 311; m/z found, 311/313 [M+H]⁺.

### Step C: ethyl 2-(5-bromo-1-methyl-1H-pyrrolo[2,3-c]pyridin-3-yl)acetate

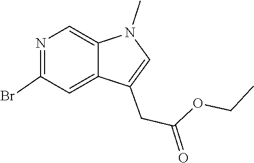 [SMILES: CCOC(=O)Cc1cn(C)c2cnc(Br)cc12]

To a solution of ethyl 2-(5-bromo-1-methyl-1H-pyrrolo[2,3-c]pyridin-3-yl)-2-oxoacetate (1.0 g, 3.2 mmol, 1.0 eq.) and Triethylsilane (747 mg, 6.4 mmol, 2.0 eq.) in TFA (10 mL). The mixture was stirred at 55° C. for 16 hours. The resulting solution was diluted with 20 ml of water and extracted with EtOAc (20 mL×3). The combined organic layers were washed with brine, dried over Na₂SO₄ and concentrated under reduced pressure. The residue was purified with flash column chromatography on silica gel (EtOAc in PE, from 0% to 20%) to afford ethyl 2-(5-bromo-1-methyl-1H-pyrrolo[2,3-c]pyridin-3-yl)acetate (350 mg, 36.6% yield) as a white solid. LC-MS (ESI): mass calcd. for C₁₂H₁₃BrN₂O₂, 297; m/z found, 297/298 [M+H]⁺.

### Step D: 3-(5-bromo-1-methyl-1H-pyrrolo[2,3-c]pyridin-3-yl)piperidine-2,6-dione

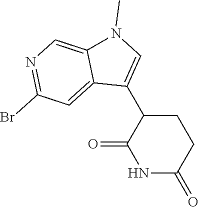 [SMILES: Cn1cc(C2CCC(=O)NC2=O)c2cc(Br)ncc21]

To a solution of ethyl 2-(5-bromo-1-methyl-1H-pyrrolo[2,3-c]pyridin-3-yl)acetate (100 mg, 0.4 mmol, 1.0 eq.) and t-BuOK (38 mg, 0.4 mmol, 1.0 eq.) in DMF (5 mL) was added acrylamide (96 mg, 1.6 mmol, 4 eq.) at 0° C. The mixture was stirred at 20° C. under N₂ for 2 hours. The resulting solution was diluted with 30 mL of water and extracted with EtOAc (20 mL×3). The combined organic layers were washed with brine, dried over Na₂SO₄ and concentrated under reduced pressure. The combined organic layers were concentrated under reduced pressure. The residue was purified with flash column chromatography on silica gel (EtOAc in PE, from 0% to 20%) to afford. 3-(5-bromo-1-methyl-1H-pyrrolo[2,3-c]pyridin-3-yl)piperidine-2,6-dione (25 mg, 23.1% yield) as a white solid.

LC-MS (ESI): mass calcd. For C₁₃H₁₂BrN₃O₂, 322; m/z found, 322/324 [M+H]⁺.

### Intermediate 87. 7-cyano-3-(2,6-dioxopiperidin-3-yl)benzofuran-5-yl trifluoromethanesulfonate

### Step A: 1-(3-bromo-2-hydroxy-5-methoxyphenyl)ethan-1-one

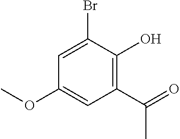

To a solution of 1-(2-hydroxy-5-methoxyphenyl)ethan-1-one (10 g, 60.2 mmol, 1.0 eq.) in AcOH (100 mL) was added the solution of Br2 (3.3 mL, 63.2 mmol, 1.05 eq.) with stirring. The mixture was stirred at 25° C. for 16 hours under N₂ atmosphere. The resulting solution was added ice water, and then filtered. The filter residue was concentrated under reduced pressure to give 1-(3-bromo-2-hydroxy-5-methoxyphenyl)ethan-1-one (12.3 g, 81% yield) as a yellow solid. LC-MS (ESI): mass calcd. for C₉H₉BrO₃, 243.97; m/z found, 245 [M+H]⁺.

### Step B: 2-bromo-1-(3-bromo-2-hydroxy-5-methoxyphenyl)ethan-1-one

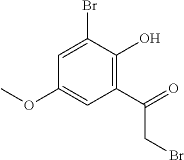

To a solution of copper(II) bromide (5.5 g, 24.5 mmol, 2.0 eq.) in Ethyl acetate (50 mL) was added the solution of 1-(3-bromo-2-hydroxy-5-methoxyphenyl)ethan-1-one (3 g, 12.2 mmol, 1.0 eq.) in CHCl₃ (30 mL) at 80° C. with stirring. The mixture was stirred at 80° C. for 16 hours under N₂ atmosphere. After cooled to room temperature, the resulting solution was filtered. The filtrate was diluted with 300 mL of water and extracted with DCM (50 mL×3). The combined organic layers were washed with NaHCO₃ (50 mL), dried over Na₂SO₄ and concentrated under reduced pressure to give 2-bromo-1-(3-bromo-2-hydroxy-5-methoxyphenyl)ethan-1-one (3.7 g, 62% yield) as a brown solid. LC-MS (ESI): mass calcd. for C₉H₈Br₂O₃, 321.88; m/z found, 323 [M+H]⁺.

### Step C: 7-bromo-5-methoxybenzofuran-3(2H)-one

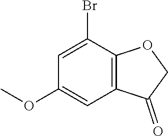

To a solution of 2-bromo-1-(3-bromo-2-hydroxy-5-methoxyphenyl)ethan-1-one (3.7 g, 11.4 mmol, 1.0 eq.) in MeCN (50.0 mL) was added K₂CO₃ (3.2 g, 22.8 mmol, 2.0 eq.) at 0° C. with stirring. The mixture was stirred at 25° C. for 40 mins under N₂. The resulting solution was filtered. The filtrate was concentrated under reduced pressure. The residue was purified by flash column chromatography on silica gel (100-200 mesh silica gel, 0-10% EtOAc in PE) to give 7-bromo-5-methoxybenzofuran-3(2H)-one (647 mg, 20% yield) as a yellow solid.

LC-MS (ESI): mass calcd. for C₁₁H₁₀INO₃, 330.97; m/z found, 332 [M+H]⁺.

### Step D: ethyl 2-(7-bromo-5-methoxybenzofuran-3-yl)acetate

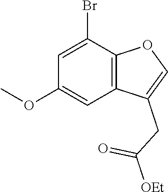 [SMILES: CCOC(=O)Cc1coc2c(Br)cc(OC)cc12]

To a solution of 7-bromo-5-methoxybenzofuran-3(2H)-one (3.6 g, 14.8 mmol, 1.0 eq.) in Toluene (100 mL) was added ethyl 2-(triphenyl-15-phosphaneylidene)acetate (10.3 g, 29.6 mmol, 2.0 eq.) with stirring. The mixture was stirred at 110° C. for 16 hours under N₂ atmosphere. After cooled to room temperature, the resulting solution was diluted with 300 mL of water and extracted with EtOAc (70 mL×3). The combined organic layers were washed with brine (50 mL), dried over Na₂SO₄ and concentrated under reduced pressure. The residue was purified by flash column chromatography on silica gel (100-200 mesh silica gel, 0-10% EtOAc in PE) to give ethyl 2-(7-bromo-5-methoxybenzofuran-3-yl)acetate (2.2 g, 46% yield) as a yellow solid. LC-MS (ESI): mass calcd. for C₁₃H₁₃BrO₄, 312.00; m/z found, 313 [M+H]⁺.

### Step E: ethyl 2-(7-cyano-5-methoxybenzofuran-3-yl)acetate

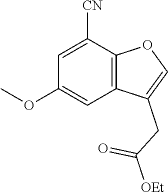 [SMILES: CCOC(=O)Cc1coc2c(C#N)cc(OC)cc12]

To a solution of ethyl 2-(7-bromo-5-methoxybenzofuran-3-yl)acetate (200 mg, 639 μmol, 1.0 eq.) in 1,4-Dioxane (5 mL) was added dppf (71 mg, 128 μmol, 0.2 eq.), Bis[tris(tert-butyl)phosphine]palladium (33 mg, 63.9 μmol, 0.1 eq.) and Zinc cyanide (150 mg, 1.3 mmol 2.0 eq.) with stirring. The mixture was stirred at 100° C. for 7 hours under N₂. After cooled to room temperature, the resulting solution was diluted with 30 mL of water and extracted with EtOAc (20 mL×3). The combined organic layers were washed with brine (50 mL), dried over Na₂SO₄ and concentrated under reduced pressure. The residue was purified by flash column chromatography on silica gel (100-200 mesh silica gel, 0-20% EtOAc in PE) to give ethyl 2-(7-cyano-5-methoxybenzofuran-3-yl)acetate (140 mg, 84.6% yield) as a white solid.

LC-MS (ESI): mass calcd. for C₁₄H₁₃NO₄, 259.08; m/z found, 260 [M+H]⁺.

### Step F: ethyl 2-(7-cyano-5-hydroxybenzofuran-3-yl)acetate

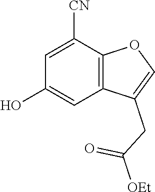 [SMILES: CCOC(=O)Cc1coc2c(C#N)cc(O)cc12]

To a solution of ethyl 2-(7-cyano-5-methoxybenzofuran-3-yl)acetate (2.1 g, 8.1 mmol, 1.0 eq.) in DCM (20.0 mL) was added tribromoborane (3.8 mL, 40.5 mmol, 5.0 eq.) at 0° C. with stirring. The mixture was stirred at 25° C. for 30 mins under N₂. The resulting solution was added in 200 mL MeOH. diluted with 300 mL of water and extracted with DCM (50 mL×3). The combined organic layers were washed with brine (50 mL), dried over Na₂SO₄ and concentrated under reduced pressure. The residue was purified by flash column chromatography on silica gel (100-200 mesh silica gel, 0-40% EtOAc in PE) to give ethyl 2-(7-cyano-5-hydroxybenzofuran-3-yl)acetate (670 mg, 33% yield) as a white solid.

LC-MS (ESI): mass calcd. for C₁₃H₁₁NO₄, 245.07; m/z found, 246 [M+H]⁺.

### Step G: 3-(2,6-dioxopiperidin-3-yl)-5-hydroxybenzofuran-7-carbonitrile

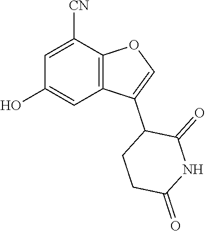 [SMILES: N#Cc1cc(O)cc2c(C3CCC(=O)NC3=O)coc12]

To a solution of ethyl 2-(7-cyano-5-hydroxybenzofuran-3-yl)acetate (600 mg, 2.4 mmol, 1.0 eq.) in DMF (6 mL) was added acrylamide (348 mg, 4.5 mmol, 2.0 eq.) and t-BuOK (347 mg, 4.9 mmol, 2.0 eq.) at 0° C. The mixture was stirred at 0° C. for 3 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-50% EtOAc in PE) to give 3-(2,6-dioxopiperidin-3-yl)-5-hydroxybenzofuran-7-carbonitrile (50 mg, 7% yield) as a yellow solid.

LC-MS (ESI): mass calcd. for C₁₄H₁₀N₂O₄, 270.06; m/z found, 271 [M+H]⁺.

### Step H: 7-cyano-3-(2,6-dioxopiperidin-3-yl)benzofuran-5-yl trifluoromethanesulfonate

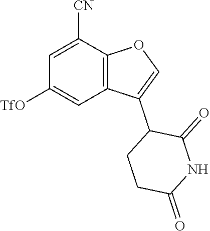 [SMILES: N#Cc1cc(OS(=O)(=O)C(F)(F)F)cc2c(C3CCC(=O)NC3=O)coc12]

A mixture of 3-(2,6-dioxopiperidin-3-yl)-5-hydroxybenzofuran-7-carbonitrile (40 mg, 148 μmol, 1.0 eq.), TEA (60 μL, 148 μmol, 1.0 eq.) and Phenyl triflimide (53 mg, 148 μmol, 1.0 eq.) in DCM (3 mL). The mixture was stirred at 25° C. for 1 hour under N₂. The resulting solution was diluted with 200 mL of water and extracted with EtOAc (200 mL×3). The combined organic layers were washed with brine (200 mL), dried over Na₂SO₄ and concentrated under reduced pressure. The residue was purified by flash column chromatography on silica gel (100-200 mesh silica gel, 0-25% EtOAc in PE) to give 7-cyano-3-(2,6-dioxopiperidin-3-yl)benzofuran-5-yl trifluoromethanesulfonate (20 mg, 33.6% yield) as a yellow solid. LC-MS (ESI): mass calcd. for C₁₅H₉F₃N₂O₆S, 402.01; m/z found, 403 [M+H]⁺.

### Intermediate 88. ethyl 2-(5-bromo-6-fluorobenzofuran-3-yl)acetate

### Step A: 2-bromo-1-(5-bromo-4-fluoro-2-hydroxyphenyl)ethan-1-one

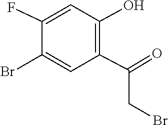 [SMILES: O=C(CBr)c1cc(Br)c(F)cc1O]

To a flask containing 1-(5-bromo-4-fluoro-2-hydroxyphenyl)ethan-1-one (500 mg, 2.15 mmol, 1 eq.) in EtOAc (10 mL) and CHCl₃ (10 mL) was added CuBr₂ (958 mg, 4.29 mmol, 2 eq.) at room temperature. The mixture was stirred at 80° C. for 16 hours under N₂ atmosphere.

The reaction was monitored by LCMS. The resulting solution was diluted with 50 ml of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford 2-bromo-1-(5-bromo-4-fluoro-2-hydroxyphenyl)ethan-1-one (400 mg, yield 60%) as a white solid. LC-MS (ESI): mass calcd. for C₈H₅Br₂FO₂, 309.8; m/z found, 311.0 [M+H]⁺.

### Step B: 5-bromo-6-fluorobenzofuran-3(2H)-one

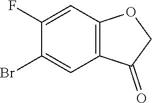 [SMILES: O=C1COc2cc(F)c(Br)cc21]

To a solution of 2-bromo-1-(5-bromo-4-fluoro-2-hydroxyphenyl)ethan-1-one (220 mg, 705 μmol, 1 eq.) in MeOH (5 mL) was added Sodium acetate (231 mg, 2.82 mmol, 4 eq.) at 25° C. The mixture was stirred at 70° C. for 2 hours under N₂ atmosphere. The reaction was monitored by LCMS. The resulting solution was diluted with 50 ml of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-10% EtOAc in PE) to afford 5-bromo-6-fluorobenzofuran-3(2H)-one (30 mg, yield 18%) as a yellow solid. ¹H NMR (400 MHz, DMSO-d₆) δ8.03 (d, J=7.3 Hz, 1H), 7.50 (d, J=9.3 Hz, 1H), 4.91 (s, 2H).

### Step C: ethyl 2-(5-bromo-6-fluorobenzofuran-3-yl)acetate

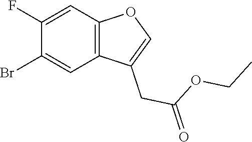 [SMILES: CCOC(=O)Cc1coc2cc(F)c(Br)cc12]

To a solution of 5-bromo-6-fluorobenzofuran-3(2H)-one (500 mg, 2.16 mmol, 1 eq.) in Toluene (5 mL) was added ethyl 2-(triphenyl-15-phosphaneylidene)acetate (754 mg, 2.16 mmol, 1 eq.) at 25° C. The mixture was stirred at 110° C. for 16 hours under N₂ atmosphere. The reaction was monitored by LCMS. The resulting solution was diluted with 50 ml of water and extracted with EtOAc. The combined organic layers were washed with brine, dried over anhydrous Na₂SO₄ and concentrated under reduced pressure. The residue was purified by flash column chromatography on silica gel (100-200 mesh silica gel, 0-10% EtOAc in PE) to afford ethyl 2-(5-bromo-6-fluorobenzofuran-3-yl)acetate (300 mg, yield 46%) as a yellow solid. ¹H NMR (400 MHz, DMSO-d₆) δ 8.01-7.96 (m, 2H), 7.78 (d, J=9.0 Hz, 1H), 4.20-4.07 (m, 2H), 3.81 (s, 2H), 1.21 (t, J=7.1 Hz, 3H).

### Intermediate 89. 6-(6-bromo-7-(difluoromethyl)-3,4-dihydroquinolin-1(2H)-yl)-4-(1,1-dioxidotetrahydro-2H-thiopyran-4-yl)-1,3-dimethyl-1,3-dihydro-2H-imidazo[4,5-c]pyridin-2-one

### Step A: 6-chloro-4-(1,1-dioxido-3,6-dihydro-2H-thiopyran-4-yl)-1,3-dimethyl-1,3-dihydro-2H-imidazo[4,5-c]pyridin-2-one

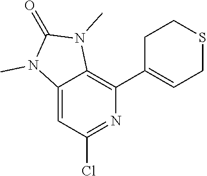 [SMILES: Cn1c(=O)n(C)c2c(C3=CCSCC3)nc(Cl)cc21]

To the solution of 2-(3,6-dihydro-2H-thiopyran-4-yl)-4,4,5,5-tetramethyl-1,3,2-dioxaborolane (2.0 g, 8.8 mmol, 1.2 eq.), 4,6-dichloro-1,3-dimethyl-1,3-dihydro-2H-imidazo[4,5-c]pyridin-2-one (1.7 g, 7.4 mmol, 1.0 eq.) and K₂CO₃ (3.1 g, 22.1 mmol, 3.0 eq.) in 1,4-Dioxane (20 mL): H₂O(2 mL) was added Pd(dppf)Cl₂ (539 mg, 737 μmol, 0.1 eq.) at r.t. The mixture was stirred at 100° C. for 6 hours under N₂. The resulting solution was diluted with 100 ml of water, then extracted with EtOAc (3*20 mL) and washed with brine (2*20 mL). The combined organic layer was dried over anhydrous sodium sulfate and concentrated under reduced pressure. The residue was purified by flash column chromatography on silica gel (100-200 mesh silica gel, 0-50% EtOAc in PE) to afford the product 6-chloro-4-(3,6-dihydro-2H-thiopyran-4-yl)-1,3-dimethyl-1,3-dihydro-2H-imidazo[4,5-c]pyridin-2-one (700 mg, 32% yield) as a yellow solid. LC-MS (ESI): mass calcd. for C₁₃H₁₄ClN₃OS, 295.05; m/z found, 296 [M+H]⁺.

### Step B: 6-chloro-4-(1,1-dioxido-3,6-dihydro-2H-thiopyran-4-yl)-1,3-dimethyl-1,3-dihydro-2H-imidazo[4,5-c]pyridin-2-one

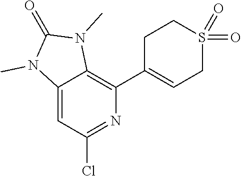 [SMILES: Cn1c(=O)n(C)c2c(C3=CCS(=O)(=O)CC3)nc(Cl)cc21]

To a solution of 6-chloro-4-(3,6-dihydro-2H-thiopyran-4-yl)-1,3-dimethyl-1,3-dihydro-2H-imidazo[4,5-c]pyridin-2-one (900 mg, 3.0 mmol, 1.0 eq.) in DCM (20 mL) was added Oxone (18.7 g, 30.4 mmol, 10.0 eq.) at room temperature. The mixture was stirred at 40° C. for 36 hours under N₂ atmosphere. The resulting solution was diluted with 100 ml of water, then extracted with DCM (3*20 mL) and washed with brine (2*20 mL). The combined organic layer was dried over anhydrous sodium sulfate and concentrated under reduced pressure. The residue was purified by flash column chromatography on silica gel (100-200 mesh silica gel, 0-30% EtOAc in PE) to afford the product 6-chloro-4-(1,1-dioxido-3,6-dihydro-2H-thiopyran-4-yl)-1,3-dimethyl-1,3-dihydro-2H-imidazo[4,5-c]pyridin-2-one (770 mg, 77% yield) as a yellow solid. LC-MS (ESI): mass calcd. for C₁₃H₁₄ClN₃O₃S, 327.04; m/z found, 328 [M+H]⁺.

### Step C: 6-(7-(difluoromethyl)-3,4-dihydroquinolin-1(2H)-yl)-4-(1,1-dioxido-3,6-dihydro-2H-thiopyran-4-yl)-1,3-dimethyl-1,3-dihydro-2H-imidazo[4,5-c]pyridin-2-one

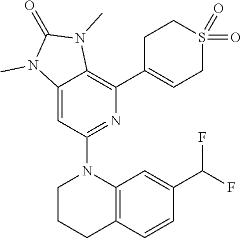 [SMILES: Cn1c(=O)n(C)c2c(C3=CCS(=O)(=O)CC3)nc(N3CCCc4ccc(C(F)F)cc43)cc21]

A mixture of 6-chloro-4-(1,1-dioxido-3,6-dihydro-2H-thiopyran-4-yl)-1,3-dimethyl-1,3-dihydro-2H-imidazo[4,5-c]pyridin-2-one (300 mg, 915 μmol, 1.0 eq.), 7-(difluoromethyl)-1,2,3,4-tetrahydroquinoline (184 mg, 1.1 mmol, 1.1 eq.), Ru-Phos (85 mg, 183 μmol, 0.2 eq.), Ru-Phos Pd G1 (67 mg, 91.5 μmol, 0.1 eq.) and Cs₂CO₃ (895 mg, 2.75 mmol, 3.0 eq.) in 1,4-Dioxane (10 mL) was stirred at 100° C. for 3 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-80% EtOAc in PE) to give 6-(7-(difluoromethyl)-3,4-dihydroquinolin-1(2H)-yl)-4-(1,1-dioxido-3,6-dihydro-2H-thiopyran-4-yl)-1,3-dimethyl-1,3-dihydro-2H-imidazo[4,5-c]pyridin-2-one (400 mg, 92% yield) as a yellow solid. LC-MS (ESI): mass calcd. for C₁₃H₁₆ClN₃O₃S, 329.06; m/z found, 330 [M+H]⁺.

### Step D: 6-(7-(difluoromethyl)-3,4-dihydroquinolin-1(2H)-yl)-4-(1,1-dioxidotetrahydro-2H-thiopyran-4-yl)-1,3-dimethyl-1,3-dihydro-2H-imidazo[4,5-c]pyridin-2-one

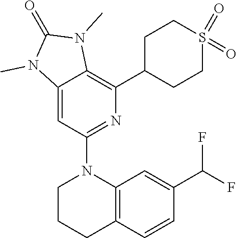 [SMILES: Cn1c(=O)n(C)c2c(C3CCS(=O)(=O)CC3)nc(N3CCCc4ccc(C(F)F)cc43)cc21]

To a solution of 6-(7-(difluoromethyl)-3,4-dihydroquinolin-1(2H)-yl)-4-(1,1-dioxido-3,6-dihydro-2H-thiopyran-4-yl)-1,3-dimethyl-1,3-dihydro-2H-imidazo[4,5-c]pyridin-2-one (400 mg, 843 μmol, 1.0 eq.) and Pd(OH)₂/C (120 mg, w.t.=10%) in MeOH (5 mL) was stirred at 25° C. for 16 hours under H₂. The solution was filtrated with celite pad to give 6-(7-(difluoromethyl)-3,4-dihydroquinolin-1(2H)-yl)-4-(1,1-dioxidotetrahydro-2H-thiopyran-4-yl)-1,3-dimethyl-1,3-dihydro-2H-imidazo[4,5-c]pyridin-2-one (250 mg crude) as a yellow solid. LC-MS (ESI): mass calcd. For C₂₃H₂₆F₂N₄O₃S, 476.17; m/z found, 477 [M+H]⁺.

### Step E: 6-(6-bromo-7-(difluoromethyl)-3,4-dihydroquinolin-1(2H)-yl)-4-(1,1-dioxidotetrahydro-2H-thiopyran-4-yl)-1,3-dimethyl-1,3-dihydro-2H-imidazo[4,5-c]pyridin-2-one

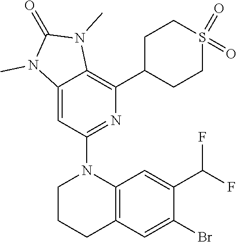 [SMILES: Cn1c(=O)n(C)c2c(C3CCS(=O)(=O)CC3)nc(N3CCCc4cc(Br)c(C(F)F)cc43)cc21]

To a solution of 6-(7-(difluoromethyl)-3,4-dihydroquinolin-1(2H)-yl)-4-(1,1-dioxidotetrahydro-2H-thiopyran-4-yl)-1,3-dimethyl-1,3-dihydro-2H-imidazo[4,5-c]pyridin-2-one (200 mg, 420 μmol, 1.0 eq.) in DCM (5 mL) was added NBS (75 mg, 420 μmol, 1.0 eq.) at 0° C.

The mixture was stirred at 0° C. for 1 hour under N₂. The resulting solution was diluted with 50 mL of water and extracted with DCM (20 mL×3). The combined organic layers were washed with brine (50 mL), dried over Na₂SO₄ and concentrated under reduced pressure. The residue was purified by flash column chromatography on silica gel (100-200 mesh silica gel, 0-50% EtOAc in PE) to give 6-(6-bromo-7-(difluoromethyl)-3,4-dihydroquinolin-1(2H)-yl)-4-(1,1-dioxidotetrahydro-2H-thiopyran-4-yl)-1,3-dimethyl-1,3-dihydro-2H-imidazo[4,5-c]pyridin-2-one (80 mg, 34% yield) as a yellow solid. LC-MS (ESI): mass calcd. For C₂₃H₂₅BrF₂N₄O₃S, 554.08; m/z found, 555 [M+H]⁺.

### Intermediate 90. 5-(4,4-difluoro-1-(7-isopropyl-1,3-dimethyl-2-oxo-2,3-dihydro-1H-benzo[d]imidazol-5-yl)-1,2,3,4-tetrahydroquinolin-6-yl) picolinic acid

### Step A: 6-bromo-2,3-dihydro-1H-spiro[quinoline-4,2′-[1,3]dithiolane]

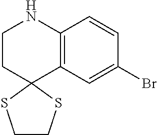 [SMILES: Brc1ccc2c(c1)C1(CCN2)SCCS1]

A solution of 6-bromo-2,3-dihydroquinolin-4(1H)-one (1.5 g, 6.6 mmol, 1.0 eq.) in Toluene (20 mL) was added p-Toluene sulfonic acid (230 mg, 1.3 mmol, 0.2 eq.), ethane-1,2-dithiol (750 mg, 8.0 mmol, 1.2 eq.). The reaction mixture was stirred at 110° C. for 16 hours. The resulting solution was quenched with 10% aqueous NaOH (20 mL) and extracted with EtOAc (30 mL×3). The combined organic layers were washed with brine (30 mL×3), dried over Na₂SO₄ and concentrated under reduced pressure. The residue was purified by silica gel chromatography (100-200 mesh silica gel, 0-50% EtOAc in PE) to give 6-bromo-2,3-dihydro-1H-spiro [quinoline-4,2′- [1,3]dithiolane](800 mg, 40% yield) as a yellow oil.

LC-MS (ESI): mass calcd. for C₁₁H₁₂BrNS₂, 300.96; m/z found, 302.2 304.2[M+H]⁺.

### Step B: methyl 5-(2,3-dihydro-1H-spiro [quinoline-4,2′- [1,3]dithiolan]-6-yl) picolinate

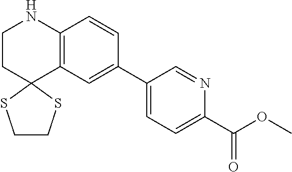 [SMILES: COC(=O)c1ccc(-c2ccc3c(c2)C2(CCN3)SCCS2)cn1]

A solution of 6-bromo-2,3-dihydro-1H-spiro[quinoline-4,2′-[1,3]dithiolane](800 mg, 2.65 mmol, 1.0 eq.) in 1,4-Dioxane (10 mL) and H₂O(1 mL) was added K₂CO₃ (1.1 g, 7.94 mmol, 3.0 eq.), methyl 5-(4,4,5,5-tetramethyl-1,3,2-dioxaborolan-2-yl)picolinate (836 mg, 3.2 mmol, 1.2 eq.) and Pd(dppf)Cl₂ (194 mg, 265 μmol, 0.1 eq.). The reaction mixture was stirred at 100° C. for 1 hour. The resulting solution was diluted with 20 ml of water and extracted with EtOAc (20 mL×3). The combined organic layers were washed with brine (20 mL×3), dried over Na₂SO₄ and concentrated under reduced pressure. The residue was purified by silica gel chromatography (100-200 mesh silica gel, 0-50% EtOAc in PE) to give methyl 5-(2,3-dihydro-1H-spiro [quinoline-4,2′- [1,3]dithiolan]-6-yl) picolinate (700 mg, 74% yield) as a yellow solid. LC-MS (ESI): mass calcd. for C₁₈H₁₈N₂O₂S₂, 358.08; m/z found, 359.0 [M+H]⁺.

### Step C: methyl 5-(1-(7-isopropyl-1,3-dimethyl-2-oxo-2,3-dihydro-1H-benzo[d]imidazol-5-yl)-2,3-dihydro-1H-spiro[quinoline-4,2′-[1,3]dithiolan]-6-yl)picolinate

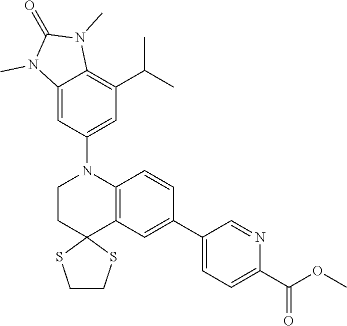

A solution of methyl 5-(2,3-dihydro-1H-spiro[quinoline-4,2′-[1,3]dithiolan]-6-yl)picolinate (200 mg, 558 μmol, 1.0 eq.) in Toluene (2 mL) was added Cs₂CO₃ (545 mg, 1.67 mmol, 3.0 eq.), 6-bromo-4-isopropyl-1,3-dimethyl-1,3-dihydro-2H-benzo[d]imidazol-2-one (175 mg, 614 μmol, 1.1 eq.), Xant-Phos (27 mg, 55.8 μmol, 0.1 eq.) and Pd₂(dba)₃ (51 mg, 55.8 μmol, 0.1 eq.). The reaction mixture was stirred at 110° C. for 16 hours. The resulting solution was diluted with 20 ml of water and extracted with EtOAc (20 mL×3). The combined organic layers were washed with brine (20 mL×3), dried over Na₂SO₄ and concentrated under reduced pressure. The residue was purified by silica gel chromatography (100-200 mesh silica gel, 0-50% EtOAc in DCM) to give methyl 5-(1-(7-isopropyl-1,3-dimethyl-2-oxo-2,3-dihydro-1H-benzo[d]imidazol-5-yl)-2,3-dihydro-1H-spiro [quinoline-4,2′- [1,3]dithiolan]-6-yl) picolinate (30 mg, 9% yield) as a yellow solid. LC-MS (ESI): mass calcd. for C₃₀H₃₂N₄O₃S₂, 560.19; m/z found, 561.2[M+H]⁺.

### Step D: methyl 5-(4,4-difluoro-1-(7-isopropyl-1,3-dimethyl-2-oxo-2,3-dihydro-1H-benzo[d]imidazol-5-yl)-1,2,3,4-tetrahydroquinolin-6-yl) picolinate

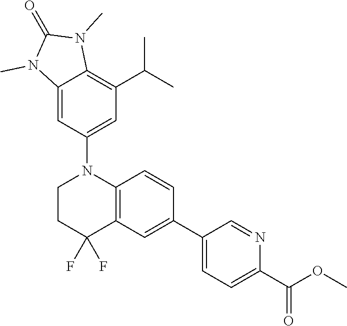

A solution of NIS (24 mg, 107 μmol, 2.0 eq.) in DCM (2 mL) was added Olah's Reagent (21 mg, 214 μmol, 4.0 eq.), methyl 5-(1-(7-isopropyl-1,3-dimethyl-2-oxo-2,3-dihydro-1H-benzo[d]imidazol-5-yl)-2,3-dihydro-1H-spiro[quinoline-4,2′-[1,3]dithiolan]-6-yl)picolinate (30 mg, 53.5 μmol, 1.0 eq.) at −70° C. The reaction mixture was stirred at −70° C. for 30 mins. The resulting solution was diluted with 20 ml of water and extracted with EtOAc (20 mL×3). The combined organic layers were washed with brine (20 mL×3), dried over Na₂SO₄ and concentrated under reduced pressure. The residue was purified by Prep-HPLC with Phenomenex SynergiYMC-Actus Triart C18 250*21 mm, and mobile phase of 20-99% ACN in water (0.1% FA) to afford methyl 5-(4,4-difluoro-1-(7-isopropyl-1,3-dimethyl-2-oxo-2,3-dihydro-1H-benzo[d]imidazol-5-yl)-1,2,3,4-tetrahydroquinolin-6-yl) picolinate (20 mg, 74% yield) as a yellow solid. LC-MS (ESI): mass calcd. for C₂₈H₂₈F₂N₄O₃, 506.21; m/z found, 507.3 [M+H]⁺.

### Step E: 5-(4,4-difluoro-1-(7-isopropyl-1,3-dimethyl-2-oxo-2,3-dihydro-1H-benzo[d]imidazol-5-yl)-1,2,3,4-tetrahydroquinolin-6-yl) picolinic acid

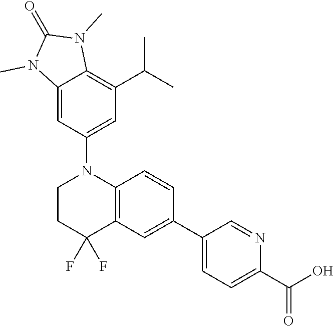 [SMILES: CC(C)c1cc(N2CCC(F)(F)c3cc(-c4ccc(C(=O)O)nc4)ccc32)cc2c1n(C)c(=O)n2C]

A solution of methyl 5-(4,4-difluoro-1-(7-isopropyl-1,3-dimethyl-2-oxo-2,3-dihydro-1H-benzo[d]imidazol-5-yl)-1,2,3,4-tetrahydroquinolin-6-yl) picolinate (20 mg, 39.5 μmol, 1.0 eq.) in THF/H₂O(2 mL) was added LiGH (2 mg, 59.2 μmol, 1.5 eq.). The reaction mixture was stirred at 25° C. for 1 hour. The resulting solution was concentrated under reduced pressure to give the product5-(4,4-difluoro-1-(7-isopropyl-1,3-dimethyl-2-oxo-2,3-dihydro-1H-benzo[d]imidazol-5-yl)-1,2,3,4-tetrahydroquinolin-6-yl) picolinic acid (15 mg, 77% yield) as a yellow solid. LC-MS (ESI): mass calcd. for C₂₇H₂₆F₂N₄O₃, 492.2; m/z found, 493.3 [M+H]⁺.

### Intermediate 91. 4-(((tert-butyldimethylsilyl)oxy)methyl)-2-iodo-1-methyl-1H-pyrrolo[3,2-c]pyridine

### Step A: 4-chloro-1-tosyl-1H-pyrrolo[3,2-c]pyridine

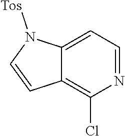

To a stirred mixture of 4-chloro-1H-pyrrolo[3,2-c]pyridine (8.5 g, 55.7 mmol, 1 eq.) in THF (80 mL) was added NaH (2.4 g, 100 mmol, 1.8 eq., w.t. =60%) at 0° C. The reaction was stirred at 0° C. for 1 hour under N₂ atmosphere. Then added a mixture of tosyl-Cl (12.7 g, 66.9 mmol, 1.2 eq.) in THF (10 mL). The reaction was stirred at 0° C. for 1 hour. The mixture was diluted with aq. NH₄Cl and diluted with 200 mL of water and extracted with EtOAc (3*200 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 4-chloro-1-tosyl-1H-pyrrolo[3,2-c]pyridine (17.0 g, yield 99.5%) as a yellow solid. LC-MS (ESI): mass calcd. for C₁₄H₁₁ClN₂O₂S, 306.0; m/z found, 307.0 [M+H]⁺.

### Step B: methyl 1-tosyl-1H-pyrrolo[3,2-c]pyridine-4-carboxylate

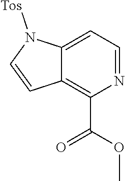

To a stirred mixture of 4-chloro-1-tosyl-1H-pyrrolo[3,2-c]pyridine (16.0 g, 52.2 mmol, 1 eq.) and Pd(dppf)Cl₂ (3.8 g, 5.22 mmol, 0.1 eq.) in MeOH (150 mL) and DMF (150 mL) was added Et3N (36.3 mL, 261 mmol, 5 eq.), the reaction mixture was stirred at 80° C. for 12 hours under the CO atmosphere. The resulting solution was diluted with 200 mL of water and extracted with EtOAc (3*200 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford the methyl 1-tosyl-1H-pyrrolo[3,2-c]pyridine-4-carboxylate (7.0 g, yield 40.6%) as a yellow solid. LC-MS (ESI): mass calcd. for C₁₆H₁₄N₂₀₄S, 330.1; m/z found, 331.1 [M+H]⁺.

### Step C: (1-tosyl-1H-pyrrolo[3,2-c]pyridin-4-yl)methanol

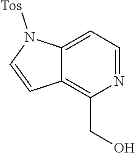

To a solution of methyl 1-tosyl-1H-pyrrolo[3,2-c]pyridine-4-carboxylate (7.0 g, 21.2 mmol, 1 eq.) in MeOH (15 mL) was added SodiumTetrahydroborate (12.0 g, 11.2 mL, 318 mmol, 15 eq.) at 0° C. The reaction was stirred at 0° C. for 1 hour under N₂ atmosphere. The mixture was diluted with aq. NH₄Cl and diluted with 150 mL of water and extracted with EtOAc (3* 150 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford (1-tosyl-1H-pyrrolo[3,2-c]pyridin-4-yl)methanol (5.0 g, yield 78.0%) as a yellow solid. LC-MS (ESI): mass calcd. for C₁₅H₁₄N₂O₃S, 302.1; m/z found, 303.1 [M+H]⁺.

### Step D: 4-(((tert-butyldimethylsilyl)oxy)methyl)-1-tosyl-1H-pyrrolo[3,2-c]pyridine

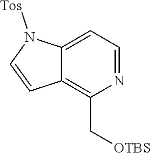

To a solution of (1-tosyl-1H-pyrrolo[3,2-c]pyridin-4-yl)methanol (4.0 g, 13.2 mmol, eq.) in DCM (160 mL) was added Imidazole (1.80 g, 1.75 mL, 26.5 mmol, 2 eq.) and tert-butylchlorodimethylsilane (2.39 g, 15.9 mmol, 1.2 eq.). The reaction was stirred at 0° C. for 30 min under N₂ atmosphere. The resulting solution was diluted with 150 mL of water and extracted with EtOAc (3* 150 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 4-(((tert-butyldimethylsilyl)oxy)methyl)-1-tosyl-1H-pyrrolo[3,2-c]pyridine (5.5 g, yield 99.8%) as a white oil. LC-MS (ESI): mass calcd. for C₂₁H₂₈N₂O₃SSi, 416.16; m/z found, 417 [M+H]⁺.

### Step E: 4-(((tert-butyldimethylsilyl)oxy)methyl)-2-iodo-1-tosyl-1H-pyrrolo[3,2-c]pyridine

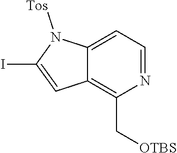

To a solution of 4-(((tert-butyldimethylsilyl)oxy)methyl)-1-tosyl-1H-pyrrolo[3,2-c]pyridine (5.5 g, 13.2 mmol, 1 eq.) in THF (50 mL) was added LDA (26.4 mL 26.4 mmol, 2 eq., 1.0 M in THF) at −78° C. The reaction was stirred at −78° C. for 1 hour under N₂ atmosphere. Then added a mixture of 12 (3.7 g, 14.5 mmol, 1.1 eq.) in THF. The reaction was stirred at −78° C. for 1 hour. LCMS showed the reaction was completed. The mixture was diluted with aq. NH₄Cl and diluted with 150 mL of water and extracted with EtOAc (3* 150 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 4-(((tert-butyldimethylsilyl)oxy)methyl)-2-iodo-1-tosyl-1H-pyrrolo[3,2-c]pyridine (2.4 g, yield 33.5%) as a yellow solid. LC-MS (ESI): mass calcd. for C₂₁H₂₇IN₂O₃SSi, 542.1; m/z found, 543.1 [M+H]⁺.

### Step F: 4-(((tert-butyldimethylsilyl)oxy)methyl)-2-iodo-1H-pyrrolo[3,2-c]pyridine

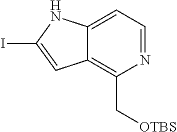 [SMILES: CC(C)(C)[Si](C)(C)OCc1nccc2[nH]c(I)cc12]

To a solution of 4-(((tert-butyldimethylsilyl)oxy)methyl)-2-iodo-1-tosyl-1H-pyrrolo[3,2-c]pyridine (2.4 g, 4.4 mmol, 1 eq.) in THF (30 mL) was added Sodium methoxide (2.4 g, 2.6 mL, 44.2 mmol, 10 eq.) at 0° C. The reaction was added a mixture of Sodium methoxide (2.4 g, 2.6 mL, 44.2 mmol, 10 eq.) in CH₃₀Na and stirred at 0° C. for 2 hours under N₂ atmosphere. The resulting solution was diluted with 80 mL of water and extracted with EtOAc (3*80 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 4-(((tert-butyldimethylsilyl)oxy)methyl)-2-iodo-1H-pyrrolo[3,2-c]pyridine (1.0 g, yield 58.2%) as a yellow solid. LC-MS (ESI): mass calcd. for C₁₄H₂₁IN₂OSi, 388.1; m/z found, 389.1 [M+H]⁺.

### Step G: 4-(((tert-butyldimethylsilyl)oxy)methyl)-2-iodo-1-methyl-1H-pyrrolo[3,2-c]pyridine

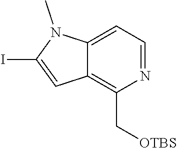 [SMILES: Cn1c(I)cc2c(CO[Si](C)(C)C(C)(C)C)nccc21]

To a solution of 4-(((tert-butyldimethylsilyl)oxy)methyl)-2-iodo-1H-pyrrolo[3,2-c]pyridine (900 mg, 2.3 mmol, 1 eq.) in THF (8 mL) was added sodium hydride (66.7 mg, 2.8 mmol, 1.2 eq.). The reaction was stirred at 0° C. for 30 min under N₂ atmosphere. Then the mixture was added iodomethane (493 mg, 3.5 mmol, 1.5 eq.). The reaction was stirred at 0° C. for 30 min under N₂ atmosphere. The resulting solution was diluted with 50 mL of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 4-(((tert-butyldimethylsilyl)oxy)methyl)-2-iodo-1-methyl-1H-pyrrolo[3,2-c]pyridine (300 mg, yield 32.2%) as a white solid. LC-MS (ESI): mass calcd. for C₁₅H₂₃IN₂OSi, 402.1; m/z found, 403.1 [M+H]⁺.

### Intermediate 92. 5-(2-chloro-7,8-dihydropyrido[3,2-d]pyrimidin-5(6H)-yl)-7-(4,4-dimethyl-1,4-azasilinan-1-yl)-1,3-dimethyl-1,6-naphthyridin-2(1H)-one

### Step A: 7-chloro-5-(2-chloro-7,8-dihydropyrido[3,2-d]pyrimidin-5(6H)-yl)-1,3-dimethyl-1,6-naphthyridin-2(1H)-one

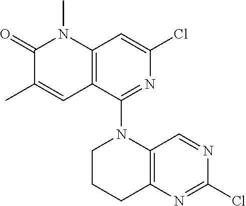 [SMILES: Cc1cc2c(N3CCCc4nc(Cl)ncc43)nc(Cl)cc2n(C)c1=O]

To a solution of 5,7-dichloro-1,3-dimethyl-1,6-naphthyridin-2(1H)-one (1.3 g, 5.37 mmol, 1.3 eq.) in 1,4-Dioxane (5 mL) was added 2-chloro-5,6,7,8-tetrahydropyrido[3,2-d]pyrimidine (700 mg, 4.13 mmol, 1 eq.), Pd₂(dba)₃ (756 mg, 825 μmol, 0.2 eq.) and potassium carbonate (1.7 g, 12.4 mmol, 3 eq.) under N₂ atmosphere. The reaction mixture was stirred at 100° C. for 16 hours. The resulting solution was diluted with 50 mL of water and extracted with EtOAc (3*50 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 7-chloro-5-(2-chloro-7,8-dihydropyrido[3,2-d]pyrimidin-5(6H)-yl)-1,3-dimethyl-1,6-naphthyridin-2(1H)-one (500 mg, yield 32.2%) as a yellow solid. LC-MS (ESI): mass calcd. for C₁₇H₁₅C₂N₅O, 375.1; m/z found, 376.0 [M+H]⁺.

### Step B: 5-(2-chloro-7,8-dihydropyrido[3,2-d]pyrimidin-5(6H)-yl)-7-(4,4-dimethyl-1,4-azasilinan-1-yl)-1,3-dimethyl-1,6-naphthyridin-2(1H)-one

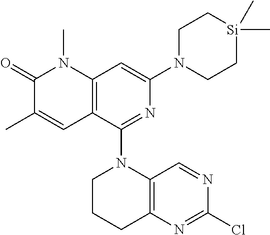

To a mixture of 7-chloro-5-(2-chloro-7,8-dihydropyrido[3,2-d]pyrimidin-5(6H)-yl)-1,3-dimethyl-1,6-naphthyridin-2(1H)-one (160 mg, 425.3 μmol, 1 eq.) in Toluene (8 mL) was added Pd₂(dba)₃ (78 mg, 85.05 μmol, 0.2 eq.), 4,4-Dimethyl-1,4-azasilinane hydrochloride (141 mg, 850.5 μmol, 2 eq.), t-BuONa (123 mg, 1.276 mmol, 3 eq.) and Xant-Phos (49 mg, 85.0 μmol, 0.2 eq.).The mixture was stirred at 100° C. for 12 hours. The resulting solution was diluted with 10 mL of water and extracted with EtOAc (3*20 mL). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford 5-(2-chloro-7,8-dihydropyrido[3,2-d]pyrimidin-5(6H)-yl)-7-(4,4-dimethyl-1,4-azasilinan-1-yl)-1,3-dimethyl-1,6-naphthyridin-2(1H)-one (30.0 mg, yield 15.0%) as a yellow solid. LC-MS (ESI): mass calcd. for C₂₃H₂₉ClN₆OSi, 468.2; m/z found, 469.2 [M+H]⁺.

### Intermediate 93. 6-chloro-1,3-dimethyl-4-(1H-pyrazol-1-yl)-1,3-dihydro-2H-imidazo[4,5-c]pyridin-2-one

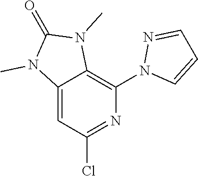 [SMILES: Cn1c(=O)n(C)c2c(-n3cccn3)nc(Cl)cc21]

To a mixture of 4,6-dichloro-1,3-dimethyl-1,3-dihydro-2H-imidazo[4,5-c]pyridin-2-one (500 mg, 2.2 mmol, 1.0 eq.) in DMF (10 mL) was added 1H-pyrazole (220 mg, 3.2 mmol, 1.5 eq.) and t-BuONa (518 mg, 5.4 mmol, 2.5 eq.) at 25° C. The mixture was stirred at 80° C. for 2 hours. The resulting solution was diluted with 50 ml of water and extracted with EtOAc (50 mL×3). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford 6-chloro-1,3-dimethyl-4-(1H-pyrazol-1-yl)-1,3-dihydro-2H-imidazo[4,5-c]pyridin-2-one (180 mg, 32% yield) as a white solid. LC-MS (ESI): mass calcd. for C₁₁H₁₀ClN₅O, 263.1; m/z found, 264.1 [M+H]⁺.

### Intermediate 94. ethyl 2-(5-bromo-2-fluorobenzofuran-3-yl)acetate

### Step A: methyl 5-bromo-2-(2-(tert-butoxy)-2-oxoethoxy)benzoate

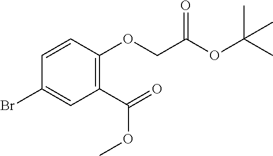 [SMILES: COC(=O)c1cc(Br)ccc1OCC(=O)OC(C)(C)C]

To a solution of methyl 5-bromo-2-hydroxybenzoate (8.0 g, 34.6 mmol, 1.0 eq.) and tert-butyl 2-bromoacetate (13.5 g, 69.2 mmol, 2.0 eq.) in MeCN (150 mL) was added K₂CO₃ (14.4 g, 104 mmol, 3.0 eq.) at room temperature. The reaction mixture was stirred at 80° C. for 3 hours. The resulting solution was diluted with 100 mL of water and extracted with EtOAc (3×100 mL) and washed with brine (200 mL). The combined organic layers were dried over anhydrous Na₂SO₄ and concentrated under reduced pressure. The residue was purified on a silica gel chromatography (100-200 mesh silica gel, 0-100% EtOAc in PE) to give methyl 5-bromo-2-(2-(tert-butoxy)-2-oxoethoxy)benzoate (8.0 g, 67% yield) as a white solid. LC-MS (ESI): mass calcd. for C₁₄H₁₇BrO₅, 344.0; m/z found, 345.0 [M+H]⁺.

### Step B: tert-butyl 5-bromo-3-oxo-2,3-dihydrobenzofuran-2-carboxylate

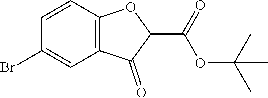 [SMILES: CC(C)(C)OC(=O)C1Oc2ccc(Br)cc2C1=O]

To a solution of t-BuOK (3.8 g, 33.8 mmol, 1.5 eq.) in THF (30 mL) was stirred for 30 minutes with ice bath. Then methyl 2-(2-(tert-butoxy)-2-oxoethoxy)benzoate (6.0 g, 22.5 mmol, 1.0 eq.) in THF (30 mL) was added dropwise while stirring. The resulting solution was stirred for 30 minutes. The reaction was quenched with 50 mL of water and extracted with EtOAc (3×60 mL) and washed with brine (100 mL). The combined organic layers were dried over anhydrous Na₂SO₄ and concentrated under reduced pressure. The residue was purified on a silica gel chromatography (100-200 mesh silica gel, 0-100% EtOAc in PE) to give tert-butyl 5-bromo-3-oxo-2,3-dihydrobenzofuran-2-carboxylate (3.2 g, 59% yield) as a white solid. LC-MS (ESI): mass calcd. for C₁₃H₁₃BrO₄, 312.0; m/z found, 313.0 [M+H]⁺.

### Step C: tert-butyl 5-bromo-3-(2-ethoxy-2-oxoethyl)benzofuran-2-carboxylate

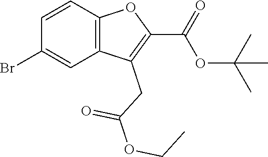 [SMILES: CCOC(=O)Cc1c(C(=O)OC(C)(C)C)oc2ccc(Br)cc12]

To a solution of tert-butyl 5-bromo-3-oxo-2,3-dihydrobenzofuran-2-carboxylate (3.2 g, 10.2 mmol, 1.0 eq.) in Toluene (50 mL) was added thyl 2-(triphenyl-15-phosphaneylidene)acetate (5.3 g, 15.3 mmol, 1.5 eq.) to the reaction mixture at room temperature. The reaction mixture was stirred at 110° C. for 12 hours. The resulting solution was diluted with 50 mL of water and extracted with EtOAc (3×50 mL) and washed with brine (60 mL). The combined organic layers were dried over anhydrous Na₂SO₄ and concentrated under reduced pressure. The residue was purified on a silica gel chromatography (100-200 mesh silica gel, 0-100% EtOAc in PE) to afford tert-butyl 5-bromo-3-(2-ethoxy-2-oxoethyl)benzofuran-2-carboxylate (2.0 g, 51% yield) as a white solid.. LC-MS (ESI): mass calcd. for C₁₇H₁₉BrO₅, 382.0; m/z found, 383.0 [M+H]⁺.

### Step D: 5-bromo-3-(2-ethoxy-2-oxoethyl)benzofuran-2-carboxylic acid

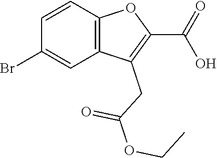 [SMILES: CCOC(=O)Cc1c(C(=O)O)oc2ccc(Br)cc12]

To a solution of tert-butyl 5-bromo-3-(2-ethoxy-2-oxoethyl)benzofuran-2-carboxylate (2.0 g, 5.2 mmol, 1.0 eq.) in DCM (6.0 mL) was added TFA (1.0 mL) at 0° C. The mixture was stirred at 25° C. for 1 hour. The reaction mixture was concentrated to give give 5-bromo-3-(2-ethoxy-2-oxoethyl)benzofuran-2-carboxylic acid (1.6 g, 94% yield) as a white solid.

LC-MS (ESI): mass calcd. for C₁₃H₁₁BrO₅, 326.0; m/z found, 327.0 [M+H]⁺.

### Step E: ethyl 2-(5-bromo-2-fluorobenzofuran-3-yl)acetate

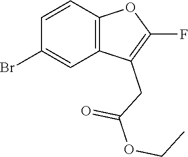 [SMILES: CCOC(=O)Cc1c(F)oc2ccc(Br)cc12]

To a solution of 5-bromo-3-(2-ethoxy-2-oxoethyl)benzofuran-2-carboxylic acid (1.6 g, 4.9 mmol, 1.0 eq.) and Selectfluor (3.5 g, 9.8 mmol, 2.0 eq.) in DCE (20 mL) and H₂O(10 mL) was added KF (1.2 g, 19.6 mmol, 4.0 eq.). at room temperature. The reaction mixture was stirred at 70° C. for 12 hours. The resulting solution was diluted with 50 mL of water and extracted with DCM (3×30 mL) and washed with brine (60 mL). The combined organic layers were dried over anhydrous Na₂SO₄ and concentrated under reduced pressure. The residue was purified on a silica gel chromatography (100-200 mesh silica gel, 0-100% EtOAc in PE) to afford ethyl 2-(5-bromo-2-fluorobenzofuran-3-yl)acetate (1.0 g, 68% yield) as a white solid..

LC-MS (ESI): mass calcd. for C₁₂H₁₀BrFO₃, 300.0; m/z found, 301.0 [M+H]⁺.

### Intermediate 95. 5-bromo-2-methylbenzofuran-3-yl acetate

### Step A: methyl 5-bromo-2-((1-methoxy-1-oxopropan-2-yl)oxy)benzoate

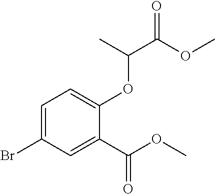 [SMILES: COC(=O)c1cc(Br)ccc1OC(C)C(=O)OC]

To a solution of methyl 5-bromo-2-hydroxybenzoate (10 g, 43.3 mmol, 1.0 eq.) and methyl 2-bromopropanoate (8.67 g, 51.9 mmol, 1.2 eq.) in acetone (150 mL) was added K₂CO₃ (17.9 g, 130 mmol, 3.0 eq.) at room temperature. The mixture was stirred reflux for 4 hours. The resulting solution was filtered through a Celite pad and filtrate was concentrated under reduced pressure to afford methyl 5-bromo-2-((1-methoxy-1-oxopropan-2-yl)oxy)benzoate (12.0 g, 87% yield) as a yellow oil. LC-MS (ESI): mass calcd. for C₁₂H₁₃BrO₅, 316.0; m/z found, 317.0 [M+H]⁺.

### Step B: 5-bromo-2-(1-carboxyethoxy)benzoic acid

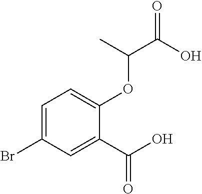 [SMILES: CC(Oc1ccc(Br)cc1C(=O)O)C(=O)O]

To a solution of methyl 5-bromo-2-((1-methoxy-1-oxopropan-2-yl)oxy)benzoate (7.0 g, 22.0 mmol, 1.0 eq.) in THF (50 mL) and MeOH (50 mL) was added NaOH (22 mL, 5M in H₂O) at room temperature. The mixture was stirred at 85° C. for 12 hours. The resulting suspension is concentrated by rotary evaporation. The residue is dissolved in water and acidified with an aqueous concentrated HCl solution under cooling with ice. The aqueous phase is extracted twice with EtOAc and the combined organic layers are washed with brine, dried over Na₂SO₄, filtered and concentrated at reduced pressure to afford 5-bromo-2-(1-carboxyethoxy)benzoic acid (5.0 g, 78% yield) as a beige solid. LC-MS (ESI): mass calcd. for C₁₀H₉BrO₅, 288.0; m/z found, 289.0 [M+H]⁺.

### Step C: 5-bromo-2-methylbenzofuran-3-yl acetate

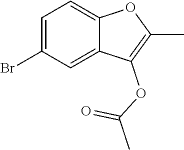

To a solution of 5-bromo-2-(1-carboxyethoxy)benzoic acid (2.0 g, 6.92 mmol, 1.0 eq.) in AcOH (20 mL) was added AcONa (1.4 g, 17.3 mmol, 2.5 eq.) at room temperature. The mixture was stirred at 150° C. for 3 hours. After cooling to room temperature, the reaction mixture is carefully quenched with ice water (20 mL). A white precipitate is formed which is collected by suction filtration, thoroughly washed with water and dried to afford 5-bromo-2-methylbenzofuran-3-yl acetate (1.0 g, 53% yield) as a white solid. LC-MS (ESI): mass calcd. for C₁₁H₉BrO₃, 268.0; m/z found, 269.0 [M+H]⁺.

### Step D: 5-bromo-2-methylbenzofuran-3-yl acetate

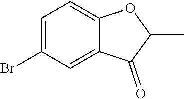

To a solution of of 5-bromo-2-methylbenzofuran-3-yl acetate (1.0 g, 3.7 mmol, 1.0 eq.) in MeOH (10 mL) was added HCl solution (5 mL, 1 M) at room temperature. The mixture was stirred at 75° C. for 3 hours. After cooling to room temperature, the reaction mixture is concentrated by rotary evaporation. The resulting solution was diluted with 20 mL of water and extracted with EtOAc (3×20 mL) and washed with brine (20 mL). The combined organic layers were dried over anhydrous Na₂SO₄ and concentrated under reduced pressure. The residue was purified on a silica gel chromatography (100-200 mesh silica gel, 0-70% EtOAc in PE) to give 5-bromo-2-methylbenzofuran-3(2H)-one (0.5 g, 59% yield) as a yellow oil.

LC-MS (ESI): mass calcd. for C₉H₇BrO₂, 226.0; m/z found, 227.0 [M+H]⁺.

### Intermediate 96. 1,3-dimethyl-2-oxo-7-(1-(tetrahydro-2H-pyran-4-carbonyl)piperidin-4-yl)-1,2-dihydroquinolin-5-yl trifluoromethanesulfonate

### Step A: tert-butyl 4-(5-methoxy-1,3-dimethyl-2-oxo-1,2-dihydroquinolin-7-yl)-3,6-dihydropyridine-1(2H)-carboxylate

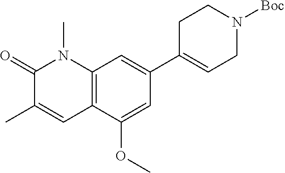 [SMILES: COc1cc(C2=CCN(C(=O)OC(C)(C)C)CC2)cc2c1cc(C)c(=O)n2C]

To a solution of 7-bromo-5-methoxy-1,3-dimethylquinolin-2(1H)-one (500 mg, 1.8 mmol, 1 eq.) in 1,4-Dioxane: H₂O(11 mL, 10:1) was added tert-butyl 4-(4,4,5,5-tetramethyl-1,3,2-dioxaborolan-2-yl)-3,6-dihydropyridine-1(2H)-carboxylate (1.1 g, 3.5 mmol, 2 eq.), Pd(dppf)Cl₂ (130 mg, 177 μmol, 0.1 eq.) and K₂CO₃ (735 mg, 5.3 mmol, 3 eq.) at 25° C. The mixture was then stirred at 100° C. for 4 hours under N₂ atmosphere. The resulting solution was diluted with 50 mL of water and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-40% EtOAc in PE) to give tert-butyl 4-(5-methoxy-1,3-dimethyl-2-oxo-1,2-dihydroquinolin-7-yl)-3,6-dihydropyridine-1(2H)-carboxylate (500 mg, 73% yield) as a yellow oil. LC-MS (ESI): mass calcd. for C₂₂H₂₈N₂O₄, 384.2; m/z found, 385.1 [M+H]⁺.

### Step B: tert-butyl 4-(5-methoxy-1,3-dimethyl-2-oxo-1,2-dihydroquinolin-7-yl)piperidine-1-carboxylate

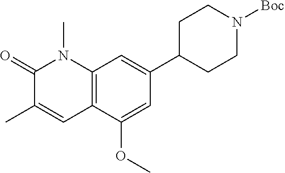 [SMILES: COc1cc(C2CCN(C(=O)OC(C)(C)C)CC2)cc2c1cc(C)c(=O)n2C]

To a solution of tert-butyl 4-(5-methoxy-1,3-dimethyl-2-oxo-1,2-dihydroquinolin-7-yl)-3,6-dihydropyridine-1(2H)-carboxylate (500 mg, 1.3 mmol, 1 eq.) in MeOH (30 mL) was added Pd/C (100 mg, 940 μmol, 0.7 eq.) at 25° C. The mixture was then stirred at 30° C. for 16 hours under H₂ atmosphere. The mixture was filtered and the filtrate was concentrated under vacuum to afford tert-butyl 4-(5-methoxy-1,3-dimethyl-2-oxo-1,2-dihydroquinolin-7-yl)piperidine-1-carboxylate (500 mg, crude) as a white solid. LC-MS (ESI): mass calcd. for C₂₂H₃₀N₂O₄, 386.2; m/z found, 387.1 [M+H]⁺.

### Step C: 5-methoxy-1,3-dimethyl-7-(piperidin-4-yl)quinolin-2(1H)-one

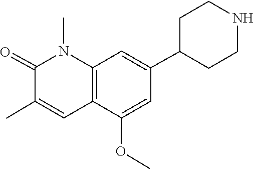 [SMILES: COc1cc(C2CCNCC2)cc2c1cc(C)c(=O)n2C]

To a solution of tert-butyl 4-(5-methoxy-1,3-dimethyl-2-oxo-1,2-dihydroquinolin-7-yl)piperidine-1-carboxylate (500 mg, 1.3 mmol, 1 eq.) in DCM: TFA (12 mL, 5:1) was added at 0° C. The mixture was then stirred at 25° C. for 1 hour under N₂ atmosphere. The mixture was filtered and the filtrate was concentrated under vacuum to afford 5-methoxy-1,3-dimethyl-7-(piperidin-4-yl)quinolin-2(1H)-one (370 mg, crude) as a yellow oil. LC-MS (ESI): mass calcd. for C₁₇H₂₂N₂O₂, 286.2; m/z found, 287.1 [M+H]⁺.

### Step D: 5-methoxy-1,3-dimethyl-7-(1-(tetrahydro-2H-pyran-4-carbonyl)piperidin-4-yl)quinolin-2(1H)-one

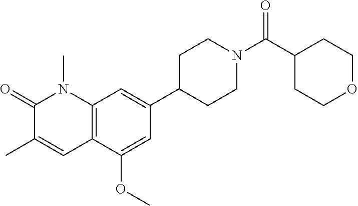 [SMILES: COc1cc(C2CCN(C(=O)C3CCOCC3)CC2)cc2c1cc(C)c(=O)n2C]

To a solution of 5-methoxy-1,3-dimethyl-7-(piperidin-4-yl)quinolin-2(1H)-one (370 mg, 1.3 mmol, 1 eq.) in DCM (10 mL) was added tetrahydro-2H-pyran-4-carbonyl chloride (384 mg, 2.6 mmol, 2 eq.) and TEA (450 μL, 3.2 mmol, 2.5 eq.) at 0° C. The mixture was then stirred at 25° C. for 1 hour under N₂ atmosphere. The resulting solution was diluted with 50 mL of water and extracted with DCM (50 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give 5-methoxy-1,3-dimethyl-7-(1-(tetrahydro-2H-pyran-4-carbonyl)piperidin-4-yl)quinolin-2(1H)-one (470 mg, 91% yield) as a white solid. LC-MS (ESI): mass calcd. for C₂₃H₃₀N₂O₄, 398.2; m/z found, 399.2 [M+H]⁺.

### Step E: 5-hydroxy-1,3-dimethyl-7-(1-(tetrahydro-2H-pyran-4-carbonyl)piperidin-4-yl)quinolin-2(1H)-one

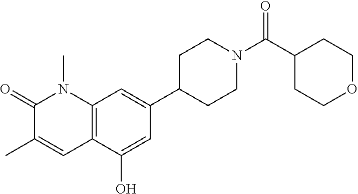 [SMILES: Cc1cc2c(O)cc(C3CCN(C(=O)C4CCOCC4)CC3)cc2n(C)c1=O]

To a solution of 5-methoxy-1,3-dimethyl-7-(1-(tetrahydro-2H-pyran-4-carbonyl)piperidin-4-yl)quinolin-2(1H)-one (430 mg, 1.1 mmol, 1 eq.) in DMF (10 mL) was added EtSNa (908 mg, 10.8 mmol, 10 eq.) at 25° C. The mixture was then stirred at 110° C. for 3 hours under N₂ atmosphere. The resulting solution was diluted with 30 mL of water and extracted with EtOAc (30 mL×3). The combined organic layers were washed with brine, dried over Na₂SO₄, and concentrated under reduced pressure. The residue was purified by flash column chromatography on silica gel (100-200 mesh silica gel, 0-5% MeOH in DCM) to give 5-hydroxy-1,3-dimethyl-7-(1-(tetrahydro-2H-pyran-4-carbonyl)piperidin-4-yl)quinolin-2(1H)-one (350 mg, 84% yield) as a white solid. LC-MS (ESI): mass calcd. for C₂₂H₂₈N₂O₄, 384.2; m/z found, 385.3 [M+H]⁺.

### Step F: 1,3-dimethyl-2-oxo-7-(1-(tetrahydro-2H-pyran-4-carbonyl)piperidin-4-yl)-1,2-dihydroquinolin-5-yl trifluoromethanesulfonate

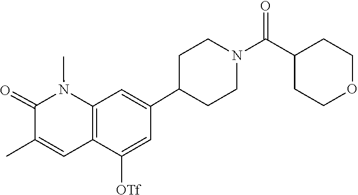 [SMILES: Cc1cc2c(OS(=O)(=O)C(F)(F)F)cc(C3CCN(C(=O)C4CCOCC4)CC3)cc2n(C)c1=O]

To a solution of 5-hydroxy-1,3-dimethyl-7-(1-(tetrahydro-2H-pyran-4-carbonyl)piperidin-4-yl)quinolin-2(1H)-one (200 mg, 520 μmol, 1 eq.) in DCM (5 mL) was added Phenyl triflimide (279 mg, 780 μmol, 1.5 eq.) and TEA (218 μL, 1.6 mmol, 3 eq.) at 0° C. The mixture was then stirred at 25° C. for 1 hour under N₂ atmosphere. The resulting solution was diluted with 50 mL of water and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-3% MeOH in DCM) to give 1,3-dimethyl-2-oxo-7-(1-(tetrahydro-2H-pyran-4-carbonyl)piperidin-4-yl)-1,2-dihydroquinolin-5-yl trifluoromethanesulfonate (150 mg, 56% yield) as a yellow oil.

LC-MS (ESI): mass calcd. for C₂₃H₂₇F₃N₂O₆S, 516.2; m/z found, 517.2 [M+H]⁺.

### Intermediate 97. 5-((7-isopropyl-1,3-dimethyl-2-oxo-2,3-dihydro-1H-benzo[d]imidazol-5-yl)(trifluoromethyl)amino)pyridin-2-yl trifluoromethanesulfonate

### Step A: 4-isopropyl-6-((6-methoxypyridin-3-yl)amino)-1,3-dimethyl-1,3-dihydro-2H-benzo[d]imidazol-2-one

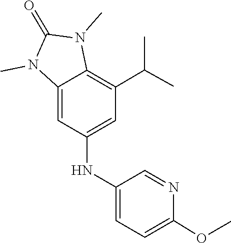

To a solution of 6-bromo-4-isopropyl-1,3-dimethyl-1,3-dihydro-2H-benzo[d]imidazol-2-one (2.5 g, 8.8 mmol, 1.0 eq.) in 1,4-Dioxane (30 mL) was added 6-methoxypyridin-3-amine (1.6 g, 13 mmol, 1.5 eq.), Ru-Phos (820 mg, 1.8 mmol, 0.2 eq.), Ru-Phos Pd G1 (1.3 g, 1.8 μmol, 0.2 eq.) and Cs₂CO₃ (8.6 g, 27 mmol, 3.0 eq.). The reaction was stirred at 100° C. for 3 hours under N₂ atmosphere. The mixture was filtered through a Celite pad and concentrated under reduced pressure. The residue was purified by flash column chromatography on silica gel (100-200 mesh silica gel, 0-100% EtOAc in PE) to afford 4-isopropyl-6-((6-methoxypyridin-3-yl)amino)-1,3-dimethyl-1,3-dihydro-2H-benzo[d]imidazol-2-one (1.5 g, 52% yield) as a yellow solid. LC-MS (ESI): mass calcd. for C₁₈H₂₂N₄O₂, 326.2; m/z found, 327.2 [M+H]⁺.

### Step B: methyl (7-isopropyl-1,3-dimethyl-2-oxo-2,3-dihydro-1H-benzo[d]imidazol-5-yl)(6-methoxypyridin-3-yl)carbamodithioate

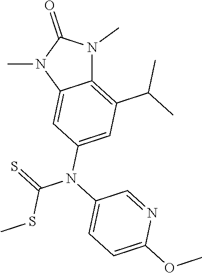

To a solution of 4-isopropyl-6-((6-methoxypyridin-3-yl)amino)-1,3-dimethyl-1,3-dihydro-2H-benzo[d]imidazol-2-one (1.2 g, 3.7 mmol, 1.0 eq.) in DMF (100 mL) was added NaH (260 mg, 7.4 mmol, 2.0 eq., w.t.=60% in oil) at 25° C. The reaction was stirred at 25° C. for 20 mins, then added CS₂ (1.1 mL, 18.4 mmol, 5.0 eq.) and stirred at 25° C. for 20 mins, then added CH₃I (1.2 mL, 18.4 mmol, 5.0 eq.). The reaction was stirred at 25° C. for 2 hours. The resulting solution was diluted with 50 mL of water and extracted with EtOAc (50 mL×3). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford methyl (7-isopropyl-1,3-dimethyl-2-oxo-2,3-dihydro-1H-benzo[d]imidazol-5-yl)(6-methoxypyridin-3-yl)carbamodithioate (1.2 g, 78% yield) as a yellow oil. LC-MS (ESI): mass calcd. for C₂₀H₂₄N₄₀₂S², 416.1; m/z found, 417.1 [M+H]⁺.

### Step C: 4-isopropyl-6-((6-methoxypyridin-3-yl)(trifluoromethyl)amino)-1,3-dimethyl-1,3-dihydro-2H-benzo[d]imidazol-2-one

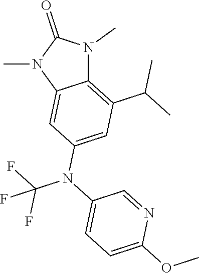

To a solution of methyl (7-isopropyl-1,3-dimethyl-2-oxo-2,3-dihydro-1H-benzo[d]imidazol-5-yl)(6-methoxypyridin-3-yl)carbamodithioate (460 mg, 1.1 mmol, 1.0 eq.) in DCM (15 mL) was added Py.HF (0.5 mL, 5.5 mmol, 5.0 eq.) and NBS (786 mg, 4.4 mmol, 4.0 eq.) at −60° C. The reaction was stirred at −60° C. for 1 hour under N₂ atmosphere. The mixture was diluted with sat. NaHSO₄ solution, extracted with EtOAc (10 mL×3) and the layers were separated. The organic layer was washed with 1 N HCl (10 mL) and brine (30 mL), dried over Na₂SO₄, filtered and concentrated. The residue was purified by flash chromatography (100-200 mesh silica gel, 0-50% EtOAc in PE) to afford 4-isopropyl-6-((6-methoxypyridin-3-yl)(trifluoromethyl)amino)-1,3-dimethyl-1,3-dihydro-2H-benzo[d]imidazol-2-one (160 mg, 37% yield) as a yellow oil. LC-MS (ESI): mass calcd. for C₁₉H₂₁F₃N₄O₂, 394.2; m/z found, 395.2 [M+H]⁺.

### Step D: 6-((6-hydroxypyridin-3-yl)(trifluoromethyl)amino)-4-isopropyl-1,3-dimethyl-1,3-dihydro-2H-benzo[d]imidazol-2-one

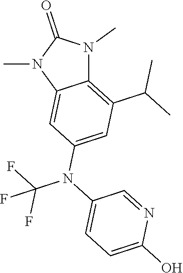 [SMILES: CC(C)c1cc(N(c2ccc(O)nc2)C(F)(F)F)cc2c1n(C)c(=O)n2C]

To a solution of 4-isopropyl-6-((6-methoxypyridin-3-yl)(trifluoromethyl)amino)-1,3-dimethyl-1,3-dihydro-2H-benzo[d]imidazol-2-one (160 mg, 406 μmol, 1.0 eq.) in MeCN (10 mL) was added TMSCl (103 μL, 811 μmol, 2.0 eq.) and KI (135 mg, 811 μmol, 2.0 eq.). The reaction was stirred at 80° C. for 2 hours. The resulting solution was diluted with 50 mL of water and extracted with EtOAc (50 mL×3). The combined organic layers were washed with brine, dried over anhydrous Na₂SO₄ and concentrated under reduced pressure. The residue was purified by flash column chromatography on silica gel (100-200 mesh silica gel, 0-10% MeOH in DCM) to afford 6-((6-hydroxypyridin-3-yl)(trifluoromethyl)amino)-4-isopropyl-1,3-dimethyl-1,3-dihydro-2H-benzo[d]imidazol-2-one (150 mg, 97% yield) as a yellow solid. LC-MS (ESI): mass calcd. for C₁₈H₁₉F₃N₄O₂, 380.1; m/z found, 381.1 [M+H]⁺.

### Step E: 5-((7-isopropyl-1,3-dimethyl-2-oxo-2,3-dihydro-1H-benzo[d]imidazol-5-yl)(trifluoromethyl)amino)pyridin-2-yl trifluoromethanesulfonate

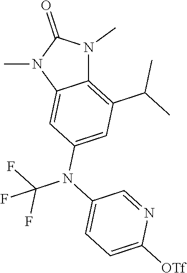 [SMILES: CC(C)c1cc(N(c2ccc(OS(=O)(=O)C(F)(F)F)nc2)C(F)(F)F)cc2c1n(C)c(=O)n2C]

To a solution of 6-((6-hydroxypyridin-3-yl)(trifluoromethyl)amino)-4-isopropyl-1,3-dimethyl-1,3-dihydro-2H-benzo[d]imidazol-2-one (130 mg, 342 μmol, 1.0 eq.) in DCM (13.0 mL) was added Tf₂O (85.9 μL, 513 μmol, 1.5 eq.) and TEA (143 μL, 1.0 mmol, 3.0 eq.) at 0° C. The reaction was stirred at 25° C. for 1 hour under N₂ atmosphere. The resulting solution was diluted with 20 mL of water and extracted with DCM (20 mL×3). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford 5-((7-isopropyl-1,3-dimethyl-2-oxo-2,3-dihydro-1H-benzo[d]imidazol-5-yl)(trifluoromethyl)amino)pyridin-2-yl trifluoromethanesulfonate (140 mg, 80% yield) as a yellow solid. LC-MS (ESI): mass calcd. for C₁₉H₁₈F₆N₄₀₄S, 512.1; m/z found, 513.1 [M+H]⁺.

### Intermediate 98. 5-bromo-7-fluorobenzofuran-3(2H)-one

### Step A: 1-(5-bromo-3-fluoro-2-hydroxyphenyl)ethan-1-one

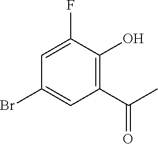

To a solution of 1-(3-fluoro-2-hydroxyphenyl)ethan-1-one (5 g, 32.4 mmol, 1.0 eq.) in MeCN (50 mL) was added Ammonium acetate (250 mg, 3.2 mmol, 0.1 eq.) and NBS (6.4 g, 35.7 mmol, 1.1 eq.) under N₂, the mixture was stirred at 25° C. for 2 hours to give yellow solution.

LCMS showed the reaction was completed. The resulting solution was diluted with 500 mL of water and extracted with EtOAc (3×300 mL). The combined organic layers were washed with brine (500 mL), dried over Na₂SO₄, and concentrated under reduced pressure. The residue was purified by flash chromatography (100-200 mesh silica gel, 0% ˜50% EtOAC in PE) to afford 1-(5-bromo-3-fluoro-2-hydroxyphenyl)ethan-1-one (6 g, yield 75%) as a yellow solid. LC-MS (ESI): mass calcd. for C₈H₆BrFO₂, 232.0/234.0; m/z found, 233.0/235.0 [M+H]⁺.

### Step B: 2-bromo-1-(5-bromo-3-fluoro-2-hydroxyphenyl)ethan-1-one

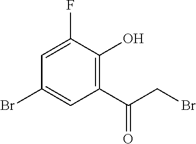 [SMILES: O=C(CBr)c1cc(Br)cc(F)c1O]

To a solution of CuBr₂ (9.8 g, 43.8 mmol, 2.0 eq.) in EtOAc (50 mL) was stirred at 80° C. under N₂, then added 1-(5-bromo-3-fluoro-2-hydroxyphenyl)ethan-1-one (5.1 g, 22 mmol, 1.0 eq.) with CHCl₃ (50 mL), the mixture was stirred at 80° C. for 16 hours to give brown solution.

LCMS showed the reaction was completed. The resulting solution was diluted with 200 mL of water and extracted with DCM (3×100 mL). The combined organic layers were washed with brine (200 mL), dried over Na₂SO₄, and concentrated under reduced pressure. The residue was purified by flash chromatography (100-200 mesh silica gel, 0% ˜30% EtOAC in PE) to afford 2-bromo-1-(5-bromo-3-fluoro-2-hydroxyphenyl)ethan-1-one (6 g, yield 70%) as a yellow oil. LC-MS (ESI): mass calcd. for C₈H₅Br₂FO₂, 309.9; m/z found, 308.9 [M−H]⁻

### Step C: 5-bromo-7-fluorobenzofuran-3(2H)-one

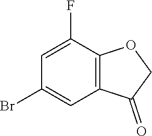 [SMILES: O=C1COc2c(F)cc(Br)cc21]

To a solution of 2-bromo-1-(5-bromo-3-fluoro-2-hydroxyphenyl)ethan-1-one (3.9 g, 12.5 mmol, 1.0 eq.) in MeOH (50 mL) was added NaOAc (4.1 g, 50 mmol, 4.0 eq.) under N₂, the mixture was stirred at 25° C. for 2 hours to give red solution. TLC showed the reaction was completed. The resulting solution was diluted with 200 mL of water and extracted with DCM (3×100 mL). The combined organic layers were washed with brine (200 mL), dried over Na₂SO₄, and concentrated under reduced pressure. The residue was purified by flash chromatography (100-200 mesh silica gel, 0% ˜30% EtOAC in PE) to afford 5-bromo-7-fluorobenzofuran-3(2H)-one (270 mg, yield 8.9%) as a yellow solid. The product was no Ms signal. ¹H NMR (400 MHz, DMSO-d₆) δ8.20-7.90 (m, 1H), 7.68 (dd, J=4.1, 1.9 Hz, 1H), 4.96 (d, J=1.3 Hz, 2H).

### Intermediate 99. 5-bromo-3-methoxy-1-methylquinolin-2(1H)-one

### Step A: 4-bromo-1-methylindoline-2,3-dione

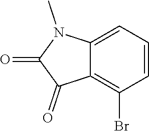

To a solution of 4-bromoindoline-2,3-dione (3.0 g, 13.3 mmol, 1.0 eq.) and K₂CO₃ (2.8 g, 19.9 mmol, 1.5 eq.) in DMF (30 mL) was added CH₃I (2.6 mL, 39.8 mmol, 3.0 eq.). The mixture was stirred for 10 minutes at room temperature and then warmed to 80° C. for 12 hours.

The resulting solution was diluted with 50 mL of water and extracted with EtOAc (3×50 mL) and washed with brine (60 mL). The combined organic layers were dried over anhydrous Na₂SO₄ and concentrated under reduced pressure. The residue was purified on a silica gel chromatography (100-200 mesh silica gel, 0-100% EtOAc in PE) to give 4-bromo-1-methylindoline-2,3-dione (2.8 g, 87% yield) as a yellow solid. LC-MS (ESI): mass calcd. for C₉H₆BrNO₂, 239.0; m/z found, 240.0 [M+H]⁺.

### Step B: 5-bromo-3-hydroxy-1-methylquinolin-2(1H)-one

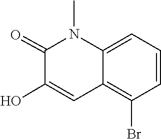

To a solution of 4-bromo-1-methylindoline-2,3-dione (1.0 g, 4.2 mmol, 1.0 eq.) and TEA (1.2 mL, 8.4 mmol, 2.0 eq.) in EtOH (15 mL) was added dropwise TMSCH₂N₂ (1.2 mL, 8.4 mmol, 2.0 eq.) at room temperature. The reaction mixture was stirred at room temperature for 12 hours. The resulting solution was diluted with 10 mL of 2 N HCl and extracted with EtOAc (3×20 mL) and washed with brine (20 mL). The combined organic layers were dried over anhydrous Na₂SO₄ and concentrated under reduced pressure. The residue was purified on a silica gel chromatography (100-200 mesh silica gel, 0-100% EtOAc in PE) to give 5-bromo-3-hydroxy-1-methylquinolin-2(1H)-one (700 mg, 66% yield) as a yellow solid.

LC-MS (ESI): mass calcd. for C₁₀HsBrNO₂, 253.0; m/z found, 254.0 [M+H]⁺.

### Step C: 5-bromo-3-methoxy-1-methylquinolin-2(1H)-one

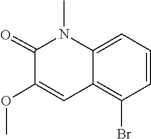

To a solution of 5-bromo-3-hydroxy-1-methylquinolin-2(1H)-one (300 mg, 1.2 mmol, 1.0 eq.) and NaH (57 mg, 2.4 mmol, 2.0 eq., w.t. =60% in oil) in DMF (10 mL) at 0° C. The reaction was stirred the same temperature for 30 minutes. CH₃I (229 μL, 3.6 mmol, 1.0 eq.) was added dropwise to the mixture. The reaction mixture was stirred at room temperature for 12 hours. The resulting solution was diluted with 20 mL of H₂O and extracted with EtOAc (3×30 mL) and washed with brine (20 mL). The combined organic layers were dried over anhydrous Na₂SO₄ and concentrated under reduced pressure. The residue was purified on a silica gel chromatography (100-200 mesh silica gel, 0-100% EtOAc in PE) to give 5-bromo-3-methoxy-1-methylquinolin-2(1H)-one (150 mg, 47% yield) as a yellow oil.

LC-MS (ESI): mass calcd. for C₁₁H₁₀BrNO₂, 267.0; m/z found, 268.0 [M+H]⁺.

### Intermediates 100. (S*)-3-(5-(3-aminoprop-1-yn-1-yl)benzofuran-3-yl)piperidine-2,6-dione TFA salt (100a) and (R*)-3-(5-(3-aminoprop-1-yn-1-yl)benzofuran-3-yl)piperidine-2,6-dione TFA salt (100b)

### Step A: ethyl 2-(5-bromobenzofuran-3-yl)acetate

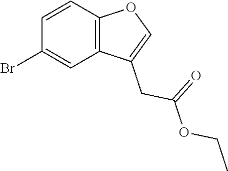 [SMILES: CCOC(=O)Cc1coc2ccc(Br)cc12]

To a solution of 5-bromobenzofuran-3(2H)-one (10 g, 46.9 mmol, 1.0 eq.) in Tol (150 mL) was added ethyl 2-(triphenyl-15-phosphaneylidene)acetate (33 g, 93.8 mmol, 2.0 eq.).

The mixture was stirred at 110° C. for 16 hours under N₂ atmosphere. After cooled to room temperature, the resulting solution was diluted with 300 mL of water and extracted with EtOAc (100 mL×3). The combined organic layers were washed with brine (50 mL), dried over Na₂SO₄ and concentrated under reduced pressure. The residue was purified by flash column chromatography on silica gel (100-200 mesh silica gel, 0-9% EtOAc in PE) to give ethyl 2-(5-bromobenzofuran-3-yl)acetate (7.3 g, yield 55%) as a yellow oil. LC-MS (ESI): mass calcd. for C₁₂H₁₁BrO₃, 281.9/283.9; m/z found, 283.2/285 [M+H]⁺.

### Step B: 3-(5-bromobenzofuran-3-yl)piperidine-2,6-dione

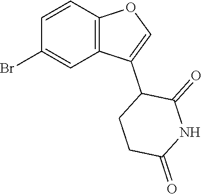 [SMILES: O=C1CCC(c2coc3ccc(Br)cc23)C(=O)N1]

To a mixture of ethyl 2-(5-bromobenzofuran-3-yl)acetate (7.3 g, 25.8 mmol, 1.0 eq.) and t-BuOK (3.5 g, 30.9 mmol, 1.2 eq.) in DMF (80 mL) was added acrylamide (2.2 g, 30.9 mmol, 1.2 eq.) at 0° C. under N₂. The mixture was stirred at 0° C. for 3 hours under N₂. The resulting solution was diluted with 300 mL of water and extracted with EtOAc (100 mL×3). The combined organic layers were washed with brine (50 mL), dried over Na₂SO₄ and concentrated under reduced pressure. The residue was purified by flash column chromatography on silica gel (100-200 mesh silica gel, 0-70% EtOAc in PE) to give 3-(5-bromobenzofuran-3-yl)piperidine-2,6-dione (4.5 g, yield 56%) as a white solid. LC-MS (ESI): mass calcd. for C₁₃H₁₀BrNO3, 306.9/308.9; m/z found, 308.1/310.1 [M+H]⁺.

### Step C: tert-butyl (3-(3-(2,6-dioxopiperidin-3-yl)benzofuran-5-yl)prop-2-yn-1-yl)carbamate

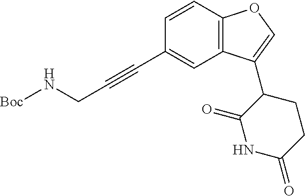 [SMILES: CC(C)(C)OC(=O)NCC#Cc1ccc2occ(C3CCC(=O)NC3=O)c2c1]

To a solution of 3-(5-bromobenzofuran-3-yl)piperidine-2,6-dione (3.5 g, 11.0 mmol, 1.0 eq.), Pd(PPh₃)C₁₂ (797 mg, 1.1 mmol, 0.1 eq.), CuI (216 mg, 1.1 mmol, 0.1 eq.) and TEA (15.5 mL, 110 mmol, 10 eq.) in DMF (40 mL) was added tert-butyl prop-2-yn-1-ylcarbamate (7.0 g, 45.4 mmol, 4.0 eq.). The mixture was stirred at 80° C. for 16 hours under N₂. After cooled to room temperature, the resulting solution was diluted with 300 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-50% EtOAc in PE) to give tert-butyl (3-(3-(2,6-dioxopiperidin-3-yl)benzofuran-5-yl)prop-2-yn-1-yl)carbamate (2.3 g, yield 31%) as a yellow solid. LC-MS (ESI): mass calcd. for C₂₁H₂₂N₂O₅, 382.1; m/z found, 381.4 [M−H]⁻. ¹HNMR (400 MHz, DMSO) δ 10.90 (s, 1H), 7.96 (s, 1H), 7.69 (s, 1H), 7.59-7.56 (m, 1H), 7.34 (dd, J=8.5, 1.6 Hz, 2H), 4.18-4.13 (m, 1H), 4.01-3.96 (m, 2H), 2.73-2.66 (m, 1H), 2.61-2.55 (m, 1H), 2.38-2.31 (m, 1H), 2.12-2.06 (m, 1H), 1.40 (s, 9H).

### Step D: tert-butyl (S*)-(3-(3-(2,6-dioxopiperidin-3-yl)benzofuran-5-yl)prop-2-yn-1-yl)carbamate and tert-butyl (R*)-(3-(3-(2,6-dioxopiperidin-3-yl)benzofuran-5-yl)prop-2-yn-1-yl)carbamate

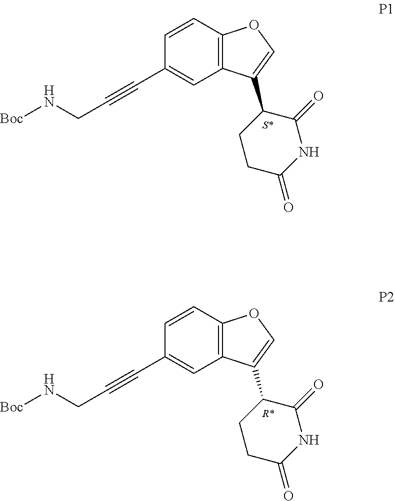 [SMILES: CC(C)(C)OC(=O)NCC#Cc1ccc2occ([C@@H]3CCC(=O)NC3=O)c2c1 ; CC(C)(C)OC(=O)NCC#Cc1ccc2occ([C@H]3CCC(=O)NC3=O)c2c1]

1 g of racemate was separated by SFC [Instrument: SHIMADZU PREP SOLUTION SFC; Column: (R,R)-WHELK, 250×20 mm I.D., 5 μm; Column temperature: 35° C.; Mobile phase: A for CO₂ and B for MeOH; Gradient: B 50%; Flow rate: 40 mL/min]to give two enantiomers; tert-butyl (S*)-(3-(3-(2,6-dioxopiperidin-3-yl)benzofuran-5-yl)prop-2-yn-1-yl)carbamate (P1, 420 mg) and tert-butyl (R*)-(3-(3-(2,6-dioxopiperidin-3-yl)benzofuran-5-yl)prop-2-yn-1-yl)carbamate (P2, 420 mg).

### Step E: (S*)-3-(5-(3-aminoprop-1-yn-1-yl)benzofuran-3-yl)piperidine-2,6-dione TFA salt (100a) and (R*)-3-(5-(3-aminoprop-1-yn-1-yl)benzofuran-3-yl)piperidine-2,6-dione TFA salt (100b)

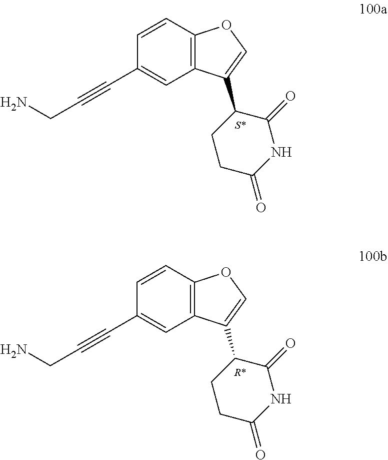 [SMILES: NCC#Cc1ccc2occ([C@@H]3CCC(=O)NC3=O)c2c1 ; NCC#Cc1ccc2occ([C@H]3CCC(=O)NC3=O)c2c1]

To a solution of tert-butyl (S*)-(3-(3-(2,6-dioxopiperidin-3-yl)benzofuran-5-yl)prop-2-yn-1-yl)carbamate or tert-butyl (R*)-(3-(3-(2,6-dioxopiperidin-3-yl)benzofuran-5-yl)prop-2-yn-1-yl)carbamate (200 mg) in DCM (10 mL) was added TFA (1 mL) at 0° C. and stirred at this temperature for 2 hours. The mixture was concentrated under reduced pressure which was directly used for next step to give (S*)-3-(5-(3-aminoprop-1-yn-1-yl)benzofuran-3-yl)piperidine-2,6-dione TFA salt or (R*)-3-(5-(3-aminoprop-1-yn-1-yl)benzofuran-3-yl)piperidine-2,6-dione TFA salt as a yellow oil. LC-MS (ESI): mass calcd. for C₁₆H₁₄N₂O₃, 282.1; m/z found, 283 [M+H]⁺.

### Intermediate 101. tert-Butyl (3-(4-(2,6-dioxopiperidin-3-yl)benzofuran-2-yl)prop-2-yn-1-yl)carbamate

### Step A: 4-bromo-2-iodobenzofuran

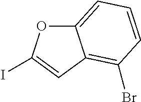 [SMILES: Brc1cccc2oc(I)cc12]

To a mixture of 4-bromobenzofuran (10.0 g, 50.8 mmol, 1.0 eq.) in THF (100 mL) was added LDA (35.0 mL, 1.2 eq., 2.0 μmol/L in THF) at −78° C. The mixture was stirred at −78° C. for 30 min, Then 12 (15.5 g, 60.9 mmol, 1.2 eq.) was added to this mixture at −78° C. The mixture was stirred at room temperature for 1 hour. The resulting solution was diluted with 100 mL of NH₄Cl solution and extracted with EtOAc (400 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give 4-bromo-2-iodobenzofuran (13.0 g, 79% yield) as a solid. LC-MS (ESI): mass calcd. for C₈H₄BrIO, 321.8; m/z found, 322.9 [M+H]⁺.

### Step B: tert-butyl (3-(4-bromobenzofuran-2-yl)prop-2-yn-1-yl)carbamate

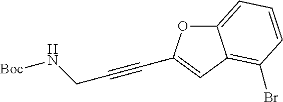 [SMILES: CC(C)(C)OC(=O)NCC#Cc1cc2c(Br)cccc2o1]

To a solution of 4-bromo-2-iodobenzofuran (13.0 g, 40.3 mmol, 1.0 eq.), TEA (56.1 mL, 10.0 eq.), Pd(PPh₃)Cl2 (2.8 g, 4.03 mmol, 0.1 eq.) and CuI (767 mg, 4.03 mmol, 0.1 eq.) in DMF (100 mL) was added tert-butyl prop-2-yn-1-ylcarbamate (7.5 g, 48.3 mmol, 1.2 eq.) at room temperature. The mixture was stirred at room temperature for 3 hours under N₂ atmosphere. The resulting solution was diluted with 200 mL of H₂O and extracted with EtOAc (200 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give to give tert-butyl (3-(4-bromobenzofuran-2-yl)prop-2-yn-1-yl)carbamate (10.5 g, 75% yield) as a yellow solid. LC-MS (ESI): mass calcd. for C₁₆H₁₆BrNO₃, 349.0; m/z found, 350.1 [M+H]⁺.

### Step C: tert-butyl (3-(4-(isoxazol-4-yl)benzofuran-2-yl)prop-2-yn-1-yl)carbamate

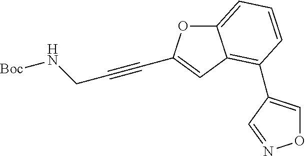 [SMILES: CC(C)(C)OC(=O)NCC#Cc1cc2c(-c3cnoc3)cccc2o1]

To a mixture of tert-butyl (3-(4-bromobenzofuran-2-yl)prop-2-yn-1-yl)carbamate (10.5 g, 30.1 mmol, 1.0 eq.), Pd(dppf)Cl₂ (2.2 g, 3.0 mmol, 0.1 eq.) and 4-(4,4,5,5-tetramethyl-1,3,2-dioxaborolan-2-yl)isoxazole (7.0 g, 36.1 mmol, 1.2 eq.) in DMSO (100 mL) and H₂O(10 mL) was added KF (5.2 g, 90.2 mmol, 3.0 eq.) at room temperature. The mixture was stirred at 90° C. for 3 hours under N₂ atmosphere. The resulting solution was diluted with 50 mL of H₂O and extracted with EtOAc (200 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give tert-butyl (3-(4-(isoxazol-4-yl)benzofuran-2-yl)prop-2-yn-1-yl)carbamate (6.5 g, crude) as a brown solid. LC-MS (ESI): mass calcd. for C₁₉H₁₈N₂O₄, 338.1; m/z found, 339.2 [M+H]⁺.

### Step D: tert-butyl (3-(4-(cyanomethyl)benzofuran-2-yl)prop-2-yn-1-yl)carbamate

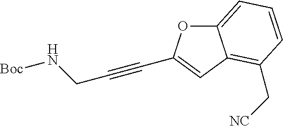 [SMILES: CC(C)(C)OC(=O)NCC#Cc1cc2c(CC#N)cccc2o1]

To a mixture of tert-butyl (3-(4-(isoxazol-4-yl)benzofuran-2-yl)prop-2-yn-1-yl)carbamate (6.5 g, 19.2 mmol, 1.0 eq.) in MeOH (45 mL) and H₂O(15 mL) was added KF (3.3 g, 57.6 mmol, 3.0 eq.) at room temperature. The mixture was then stirred at 90° C. for 30 mins under N₂ atmosphere. The resulting solution was diluted with 50 mL of H₂O and extracted with EtOAc (100 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give tert-butyl (3-(4-(cyanomethyl)benzofuran-2-yl)prop-2-yn-1-yl)carbamate (3.2 g, 31.4% yield two steps) as a yellow solid. LC-MS (ESI): mass calcd. for C₁₈H₁₈N₂O₃, 310.1; m/z found, 311.2 [M+H]⁺.

### Step E: methyl 4-(2-(3-((tert-butoxycarbonyl)amino)prop-1-yn-1-yl)benzofuran-4-yl)-4-cyanobutanoate

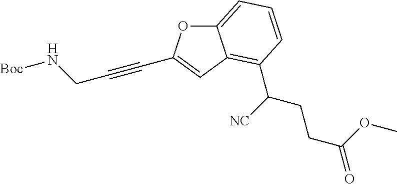 [SMILES: COC(=O)CCC(C#N)c1cccc2oc(C#CCNC(=O)OC(C)(C)C)cc12]

To a mixture of tert-butyl (3-(4-(cyanomethyl)benzofuran-2-yl)prop-2-yn-1-yl)carbamate (2.0 g, 6.5 mmol, 1.0 eq.) in DMF (20 mL) was added K₂CO₃ (2.7 g, 19.5 mmol, 3.0 eq.) and methyl acrylate (731.0 mg, 8.5 mmol, 1.3 eq.) at room temperature. The mixture was then stirred at 50° C. for 1 hour under N₂ atmosphere. The resulting solution was diluted with 50 mL of H₂O and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give ethyl 4-(2-(3-((tert-butoxycarbonyl)amino)prop-1-yn-1-yl)benzofuran-4-yl)-4-cyanobutanoate (800 mg, 31.4% yield) as a yellow solid. LC-MS (ESI): mass calcd. for C₂₂H₂₄N₂O₅, 396.2; m/z found, 397.3 [M+H]⁺.

### Step F: methyl 4-(2-(3-((tert-butoxycarbonyl)amino)prop-1-yn-1-yl)benzofuran-4-yl)-4-cyanobutanoate

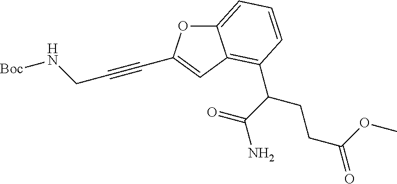 [SMILES: COC(=O)CCC(C(N)=O)c1cccc2oc(C#CCNC(=O)OC(C)(C)C)cc12]

To a mixture of methyl 4-(2-(3-((tert-butoxycarbonyl)amino)prop-1-yn-1-yl)benzofuran-4-yl)-4-cyanobutanoate (800 mg, 2.0 mmol, 1.0 eq.) in Tol (10 mL) was added InCl3 (45 mg, 0.2 mmol, 0.1 eq.) and (E)-acetaldehyde oxime (358 mg, 6.0 mmol, 3.0 eq.) at room temperature. The mixture was stirred at 110° C. for 2 hours under N₂ atmosphere. The resulting solution was diluted with 50 mL of H₂O and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give methyl 5-amino-4-(2-(3-((tert-butoxycarbonyl)amino)prop-1-yn-1-yl)benzofuran-4-yl)-5-oxopentanoate (600 mg, 72% yield) as a white solid. LC-MS (ESI): mass calcd. for C₂₂H₂₆N₂O₆, 414.2; m/z found, 415.3 [M+H]⁺.

### Step G: tert-butyl (3-(4-(2,6-dioxopiperidin-3-yl)benzofuran-2-yl)prop-2-yn-1-yl)carbamate

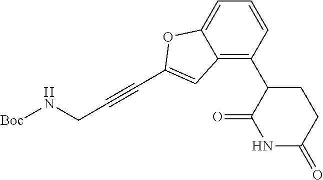 [SMILES: CC(C)(C)OC(=O)NCC#Cc1cc2c(C3CCC(=O)NC3=O)cccc2o1]

To a solution of methyl 5-amino-4-(2-(3-((tert-butoxycarbonyl)amino)prop-1-yn-1-yl)benzofuran-4-yl)-5-oxopentanoate (600 mg, 1.4 mmol, 1.0 eq.) in THF (10 mL) was added t-BuOK (157 mg, 1.4 mmol, 1.0 eq.) at 0° C. with stirring. The mixture was stirred at 0° C. for 30 min under N₂ atmosphere. The resulting solution was diluted with 50 mL of H₂O and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give tert-butyl(3-(4-(2,6-dioxopiperidin-3-yl)benzofuran-2-yl)prop-2-yn-1-yl)carbamate (400 mg, 72% yield) as a white solid. LC-MS (ESI): mass calcd. for C₂₁H₂₂N₂O₅, 382.2; m/z found, 383.3 [M+H]⁺. ¹H NMR (400 MHz, DMSO-d₆) δ 10.89 (s, 1H), 7.51-7.42 (m, 2H), 7.37-7.33 (m, 1H), 7.27 (s, 1H), 7.13 (d, J=7.3 Hz, 1H), 4.22 (dd, J=12.2, 4.9 Hz, 1H), 4.09-4.03 (m, 2H), 2.76-2.67 (m, 1H), 2.58-2.52 (m, 1H), 2.39-2.31 (m, 1H), 2.07-2.00 (m, 1H), 1.41 (s, 9H).

### Intermediates 102. (R*)-3-(2-(3-aminoprop-1-yn-1-yl)benzofuran-4-yl)piperidine-2,6-dione (102a) and (S*)-3-(2-(3-aminoprop-1-yn-1-yl)benzofuran-4-yl)piperidine-2,6-dione (102b)

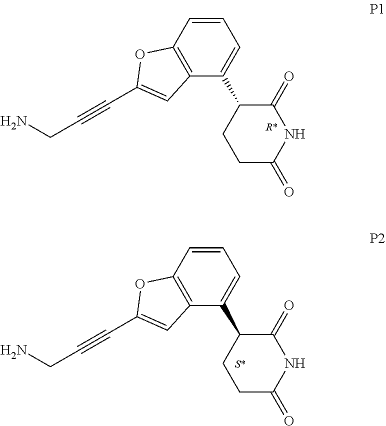 [SMILES: NCC#Cc1cc2c([C@H]3CCC(=O)NC3=O)cccc2o1 ; NCC#Cc1cc2c([C@@H]3CCC(=O)NC3=O)cccc2o1]

### Step A: tert-butyl (R*)-(3-(4-(2,6-dioxopiperidin-3-yl)benzofuran-2-yl)prop-2-yn-1-yl)carbamate and tert-butyl (S*)-(3-(4-(2,6-dioxopiperidin-3-yl)benzofuran-2-yl)prop-2-yn-1-yl)carbamate

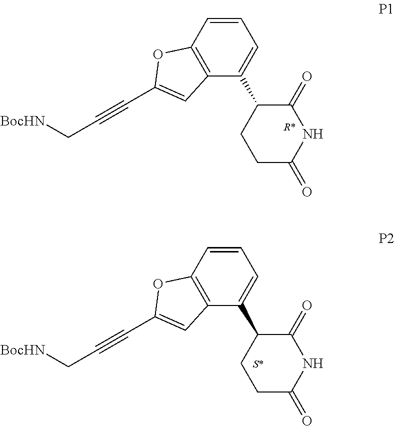 [SMILES: CC(C)(C)OC(=O)NCC#Cc1cc2c([C@H]3CCC(=O)NC3=O)cccc2o1 ; CC(C)(C)OC(=O)NCC#Cc1cc2c([C@@H]3CCC(=O)NC3=O)cccc2o1]

Racemic tert-butyl (3-(4-(2,6-dioxopiperidin-3-yl)benzofuran-2-yl)prop-2-yn-1-yl)carbamate (5.00 g, 1 Eq, 13.1 mmol) was chiral separated by SFC [Instrument: SHIMADZU PREP SOLUTION SFC; Column: (R,R)-WHELK, 250×20 mm I.D., 5 ptm; Column temperature: 35° C.; Mobile phase: A for CO₂ and B for MeOH; Gradient: B 50%; Flow rate: 40 mL/min]to give tert-butyl (R*)-(3-(4-(2,6-dioxopiperidin-3-yl)benzofuran-2-yl)prop-2-yn-1-yl)carbamate (2.10 g, 5.49 mmol, 42.0%) and tert-butyl (S*)-(3-(4-(2,6-dioxopiperidin-3-yl)benzofuran-2-yl)prop-2-yn-1-yl)carbamate (2.20 g, 5.75 mmol, 44.0%) as a yellow solid.

### Step B: (R*)-3-(2-(3-aminoprop-1-yn-1-yl)benzofuran-4-yl)piperidine-2,6-dione (102a) and (S*)-3-(2-(3-aminoprop-1-yn-1-yl)benzofuran-4-yl)piperidine-2,6-dione (102b)

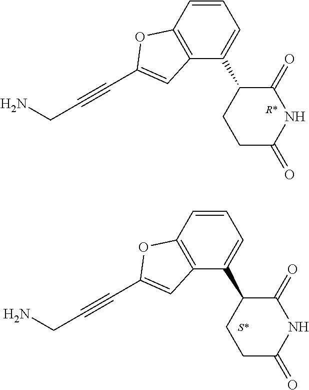 [SMILES: NCC#Cc1cc2c([C@H]3CCC(=O)NC3=O)cccc2o1 ; NCC#Cc1cc2c([C@@H]3CCC(=O)NC3=O)cccc2o1]

To a solution of tert-butyl (S tert-butyl (3-(4-(2,6-dioxopiperidin-3-yl)benzofuran-2-yl)prop-2-yn-1-yl)carbamate or tert-butyl (R*)-tert-butyl (3-(4-(2,6-dioxopiperidin-3-yl)benzofuran-2-yl)prop-2-yn-1-yl)carbamate (200 mg) in DCM (100 mL) was added TFA (1 mL) at 0° C. and stirred at this temperature for 2 hours. The mixture was concentrated under reduced pressure which was directly used for next step to give (R*)-3-(2-(3-aminoprop-1-yn-1-yl)benzofuran-4-yl)piperidine-2,6-dione TFA salt or (S*)-3-(2-(3-aminoprop-1-yn-1-yl)benzofuran-4-yl)piperidine-2,6-dione TFA salt as a yellow oil. LC-MS (ESI): mass calcd. for C₁₆H₁₄N₂O₃, 282.1; m/z found, 283 [M+H]⁺.

### Example A1. N-(3-(1-(2,6-Dioxopiperidin-3-yl)-7-fluoro-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: 3-(6-Bromo-7-fluoro-]H-indazol-1-yl)piperidine-2,6-dione

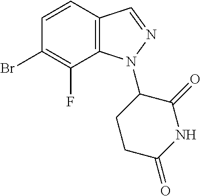 [SMILES: O=C1CCC(n2ncc3ccc(Br)c(F)c32)C(=O)N1]

To a mixture of 6-bromo-7-fluoro-1H-indazole (900 mg, 4.2 mmol, 1 eq.) and 3-bromopiperidine-2,6-dione (1.6 g, 8.4 mmol, 2 eq.) in THF (15 mL) was added t-BuOK (1.4 g, 12.6 mmol, 3 eq.) at room temperature under N₂ atmosphere. The reaction mixture was stirred at 50° C. for 16 hours. The mixture was then diluted with water and extracted with EtOAc. The combined organic layers were washed with brine, dried over anhydrous Na₂SO₄, and concentrated under reduced pressure. The residue was purified by flash column chromatography on silica gel (100-200 mesh silica gel, 0-50% EtOAc in PE) to give 3-(6-bromo-7-fluoro-1H-indazol-1-yl)piperidine-2,6-dione (250 mg, 18% yield) as a white solid.

LC-MS (ESI): mass calcd. for C₁₂H₉BrFN₃O₂, 325.0/327.0; m/z found, 326.0/328.0 [M+H]⁺.

### Step B: tert-Butyl (3-(1-(2,6-dioxopiperidin-3-yl)-7-fluoro-1H-indazol-6-yl)prop-2-yn-I-yl)carbamate

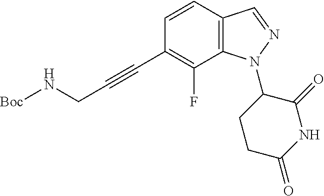 [SMILES: CC(C)(C)OC(=O)NCC#Cc1ccc2cnn(C3CCC(=O)NC3=O)c2c1F]

To a mixture of 3-(6-bromo-7-fluoro-1H-indazol-1-yl)piperidine-2,6-dione (120 mg, 368 μmol, 1 eq.) and tert-butyl prop-2-yn-1-ylcarbamate (86 mg, 552 μmol, 1.5 eq.) in DMF (8 mL) and TEA (1 mL, 20 eq.) was added Pd(PPh₃)C₁₂ (39 mg, 55.2 μmol, 0.15 eq.) and CuI (11 mg, 55.2 μmol, 0.15 eq) at room temperature. The reaction mixture was stirred at 80° C. for 16 hours under N₂ atmosphere. The mixture was diluted with water and extracted with EtOAc. The combined organic layers were washed with brine, dried over anhydrous Na₂SO₄, and concentrated under reduced pressure. The residue was purified by flash chromatography (100-200 mesh silica gel, 0-80% EtOAc in PE) to give tert-butyl (3-(1-(2,6-dioxopiperidin-3-yl)-7-fluoro-1H-indazol-6-yl)prop-2-yn-1-yl)carbamate (40 mg, 27% yield) as a yellow solid.

LC-MS (ESI): mass calcd. for C₂₀H₂₁FN₄O₄, 400.2; m/z found, 401.2 [M+H]⁺.

### Step C: 3-(6-(3-Aminoprop-1-yn-1-yl)-7-fluoro-]H-indazol-1-yl)piperidine-2,6-dione

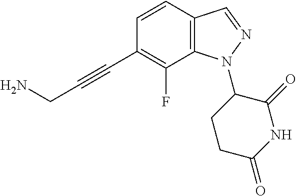 [SMILES: NCC#Cc1ccc2cnn(C3CCC(=O)NC3=O)c2c1F]

A solution of tert-butyl (3-(1-(2,6-dioxopiperidin-3-yl)-7-fluoro-1H-indazol-6-yl)prop-2-yn-1-yl)carbamate (40 mg, 100 μmol, 1 eq.) in DCM (3 mL) was treated with TFA (1 mL) at 0° C. The reaction mixture was stirred at 0° C. for 2 hours. The reaction mixture was directly concentrated under reduced pressure to give 3-(6-(3-aminoprop-1-yn-1-yl)-7-fluoro-1H-indazol-1-yl)piperidine-2,6-dione (40 mg, crude, TFA salt).

LC-MS (ESI): mass calcd. for C₁₅H₁₃FN₄O₂, 300.1; m/z found, 301.1 [M+H]⁺.

### Step D: N-(3-(1-(2,6-Dioxopiperidin-3-yl)-7-fluoro-]H-indazol-6-yl)prop-2-yn-1-yl)-5-(8-(7-isopropyl-4,3-dimethyl-2-oxo-2,3-dihydro-]H-benzo[d]imidazol-5-yl)isoquinolin-3-yl)picolinamide

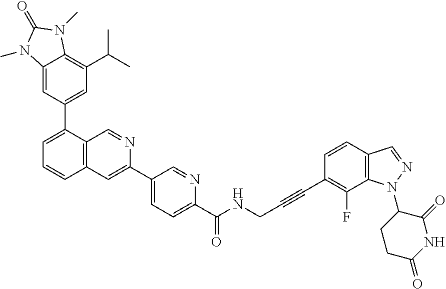 [SMILES: CC(C)c1cc(-c2cccc3cc(-c4ccc(C(=O)NCC#Cc5ccc6cnn(C7CCC(=O)NC7=O)c6c5F)nc4)ncc23)cc2c1n(C)c(=O)n2C]

To a mixture of 3-(6-(3-aminoprop-1-yn-1-yl)-7-fluoro-1H-indazol-1-yl)piperidine-2,6-dione (40 mg, 100 μmol, 1.2 eq.) and 5-(8-(7-isopropyl-1,3-dimethyl-2-oxo-2,3-dihydro-1H-benzo[d]imidazol-5-yl)isoquinolin-3-yl)picolinic acid (38 mg, 83 μmol, 1 eq.; Intermediate 2) in DMF (5 mL) was added HATU (63 mg, 166 μmol, 2 eq.) and DIPEA (57 μL, 333 μmol, 4 eq.) at 25° C. The reaction mixture was stirred at room temperature for 30 mins. The resulting solution was diluted with 20 mL of H₂O and extracted with EtOAc (20 mL×3). The combined organic layers were washed with brine, dried over Na₂SO₄, and concentrated under reduced pressure. The residue was purified by Prep-HPLC (Triant C18 5 μm column, 0-80% MeCN in H₂O with 0.1% FA) to give N-(3-(1-(2,6-dioxopiperidin-3-yl)-7-fluoro-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide (11.7 mg, 19% yield) as a yellow solid.

LC-MS (ESI): mass calcd. for C₄₂H₃₅FN₈O₄, 734.3; m/z found, 735.1 [M+H]⁺.

¹H NMR (400 MHz, DMSO-d₆) δ 11.14 (s, 1H), 9.50 (d, J=2.1 Hz, 1H), 9.44-9.35 (m, 2H), 8.88-8.72 (m, 2H), 8.33-8.17 (m, 2H), 8.10 (d, J=8.3 Hz, 1H), 7.92 (t, J=7.7 Hz, 1H), 7.68 (d, J=7.1 Hz, 1H), 7.62 (d, J=8.3 Hz, 1H), 7.28 (s, 1H), 7.23-7.17 (m, 2H), 5.90 -5.76 (m, 1H), 4.45 (d, J=5.9 Hz, 2H), 3.81-3.72 (m, 1H), 3.66 (s, 3H), 3.39 (s, 3H), 3.01-2.91 (m, 1H), 2.75-2.63 (m, 2H), 2.42-2.34 (m, 1H), 1.34 (d, J=6.7 Hz, 6H).

### Example A2. N-(3-(3-((2,4-Dioxo-3-azabicyclo[3.1.1]heptan-1-yl)amino)phenyl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: Methyl 3-cyano-3-((3-iodophenyl)amino)cyclobutane-1-carboxylate

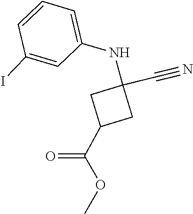 [SMILES: COC(=O)C1CC(C#N)(Nc2cccc(I)c2)C1]

To a solution of 3-iodoaniline (3.7 g, 17.2 mmol, 1.0 eq.) in EtOH (40 mL) was added methyl 3-oxocyclobutane-1-carboxylate (2 g, 15.4 mmol, 0.9 eq.) and trimethylsilylcyanide (2 mL, 15.4 mmol, 0.9 eq.). The reaction was stirred at 25° C. for 16 hours under N₂. The resulting solution was diluted with 100 mL of H₂O and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-50% EtOAc in PE) to give methyl 3-cyano-3-((3-iodophenyl)amino)cyclobutane-1-carboxylate (3.3 g, 54% yield) as a white solid.

LC-MS (ESI): mass calcd. for C₁₃H₁₃IN₂O₂, 356.0; m/z found, 357.0 [M+H]⁺.

### Step B: Methyl 3-carbamoyl-3-((3-iodophenyl)amino)cyclobutane-1-carboxylate

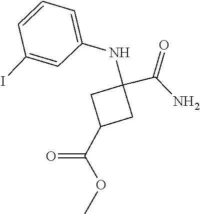 [SMILES: COC(=O)C1CC(Nc2cccc(I)c2)(C(N)=O)C1]

To a solution of methyl 3-cyano-3-((3-iodophenyl)amino)cyclobutane-1-carboxylate (2 g, 5.6 mmol, 1.0 eq.) in toluene (20 mL) was added indium trichloride (124 mg, 562 μmol, 0.1 eq.) and acetaldoxime (995 mg, 16.8 mmol, 3.0 eq.). The reaction was stirred at 110° C. for 2 hours under N₂. The mixture was concentrated under reduced pressure. The residue was purified by flash chromatography (100-200 mesh silica gel, 0-50% EtOAc in PE) to give methyl 3-carbamoyl-3-((3-iodophenyl)amino)cyclobutane-1-carboxylate (0.9 g, 43% yield) as a yellow solid.

LC-MS (ESI): mass calcd. for C₁₃H₁₅IN₂O₃, 374.0; m/z found, 375.0 [M+H]⁺.

### Step C: 1-((3-Iodophenyl)amino)-3-azabicyclo[3.1.]]heptane-2,4-dione

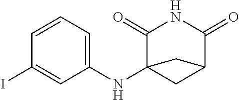 [SMILES: O=C1NC(=O)C2(Nc3cccc(I)c3)CC1C2]

To a solution of methyl 3-carbamoyl-3-((3-iodophenyl)amino)cyclobutane-1-carboxylate (500 mg, 1.3 mmol, 1.0 eq.) in MeCN (10 mL) was added benzyl(trimethyl)ammonium hydroxide (670 mg, 3.9 mmol, 3.0 eq.). The reaction was stirred at 25° C. for 1 hour under N₂. The mixture was poured into water and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give 1-((3-iodophenyl)amino)-3-azabicyclo[3.1.1]heptane-2,4-dione (90 mg, 20% yield) as a white solid.

LC-MS (ESI): mass calcd. for C₁₂H₁₁IN₂O₂, 342.0; m/z found, 343.0 [M+H]⁺.

Steps D-F: N-(3-(3-((2,4-Dioxo-3-azabicyclo[3.1.1]heptan-1-yl)amino)phenyl)prop-2-yn-1-yl)-5-(8-(7-isopropyl-1,3-dimethyl-2-oxo-2,3-dihydro-]H-benzo[d]imidazol-5-yl)isoquinolin-3-yl)picolinamide

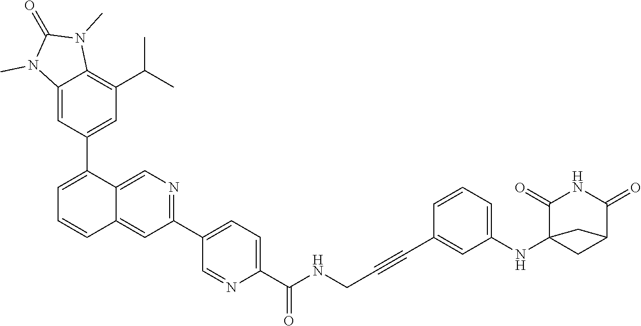 [SMILES: CC(C)c1cc(-c2cccc3cc(-c4ccc(C(=O)NCC#Cc5cccc(NC67CC(C6)C(=O)NC7=O)c5)nc4)ncc23)cc2c1n(C)c(=O)n2C]

The title compound was prepared from 1-((3-iodophenyl)amino)-3-azabicyclo[3.1.1]heptane-2,4-dione according to the procedures described in Example A1, Steps B-D. N-(3-(3-((2,4-Dioxo-3-azabicyclo[3.1.1]heptan-1-yl)amino)phenyl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide (12.8 mg, 9% yield over 3 steps) was obtained as a white solid.

LC-MS (ESI): mass calcd. for C₄₂H₃₇N₇O₄, 703.3; m/z found, 704.5 [M+H]⁺.

¹H NMR (400 MHz, DMSO-d₆) δ 10.73 (s, 1H), 9.49 (d, J=1.6 Hz, 1H), 9.40 (s, 1H), 9.29 (t, J=5.8 Hz, 1H), 8.82-8.75 (m, 2H), 8.21 (d, J=8.2 Hz, 1H), 8.10 (d, J=8.3 Hz, 1H), 7.95-7.89 (m, 1H), 7.68 (d, J=7.1 Hz, 1H), 7.28 (s, 1H), 7.21 (s, 1H), 7.08-6.99 (m, 1H), 6.64 (d, J=7.6 Hz, 1H), 6.49-6.44 (m, 2H), 6.42 (s, 1H), 4.34 (d, J=5.7 Hz, 2H), 3.80-3.71 (m, 1H), 3.66 (s, 3H), 3.39 (s, 3H), 2.99-2.94 (m, 1H), 2.79-2.72 (m, 2H), 2.49-2.45 (m, 2H), 1.35 (d, J=6.8 Hz, 6H).

### Example A3. N-(3-(1-(2,6-Dioxopiperidin-3-yl)-1H-benzo[d][1,2,3]triazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: 3-(6-Bromo-]H-benzo[d][1,2,3]triazol-1-yl)piperidine-2,6-dione and 3-(5-bromo-1H-benzo[d][1,2,3]triazol-1-yl)piperidine-2,6-dione

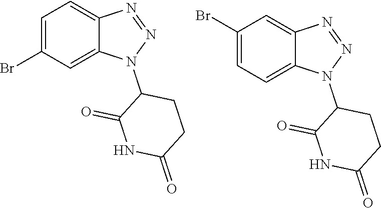 [SMILES: O=C1CCC(n2nnc3ccc(Br)cc32)C(=O)N1 ; O=C1CCC(n2nnc3cc(Br)ccc32)C(=O)N1]

To a solution of 6-bromo-1H-benzo[d][1,2,3]triazole (5 g, 25.4 mmol, 1.0 eq.) and 3-bromopiperidine-2,6-dione (9.7 g, 50.8 mmol, 2.0 eq.) in DMF (40 mL) was added t-BuOK (5.7 g, 50.8 mmol, 2.0 eq.). The mixture was stirred at 60° C. for 16 hours under N₂ atmosphere. The resulting solution was diluted with 150 mL of H₂O and extracted with EtOAc (80 mL×3). The combined organic layers were washed with brine, dried over Na₂SO₄, and concentrated under reduced pressure. The residue was purified by flash column chromatography on silica gel (100-200 mesh silica gel, 0-30% EtOAc in PE) and prep-HPLC (Triant C18 5 m column, 0-50% MeOH in H₂O with 0.1% FA) to give 3-(6-bromo-1H-benzo[d][1,2,3]triazol-1-yl)piperidine-2,6-dione (120 mg, 3% yield) and 3-(5-bromo-1H-benzo[d][1,2,3]triazol-1-yl)piperidine-2,6-dione (110 mg, 2% yield)

LC-MS (ESI): mass calcd. for C₁₁H₉BrN₄O₂, 308.0/310.0; m/z found, 309/311 [M+H]⁺.

### Steps B-D: N-(3-(1-(2,6-Dioxopiperidin-3-yl)-]H-benzo[d][ ],2,3]triazol-6-yl)prop-2-yn-1-yl)-5-(8-(7-isopropyl-1,3-dimethyl-2-oxo-2,3-dihydro-]H-benzo[d]imidazol-5-yl)isoquinolin-3-yl)picolinamide

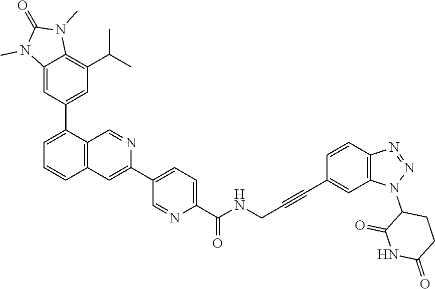 [SMILES: CC(C)c1cc(-c2cccc3cc(-c4ccc(C(=O)NCC#Cc5ccc6nnn(C7CCC(=O)NC7=O)c6c5)nc4)ncc23)cc2c1n(C)c(=O)n2C]

The title compound was prepared from 3-(6-bromo-1H-benzo[d][1,2,3]triazol-1-yl)piperidine-2,6-dione according to the procedures described in Example A1, Steps B-D. N-(3-(1-(2,6-Dioxopiperidin-3-yl)-1H-benzo[d][1,2,3]triazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide (48 mg, 16% yield over 3 steps) was obtained as a white solid.

LC-MS (ESI): mass calcd. for C₄₁H₃₅N₉O₄, 717.3; m/z found, 718.5 [M+H]⁺.

¹H NMR (400 MHz, DMSO-d₆) δ 11.27 (s, 1H), 9.54-9.46 (m, 1H), 9.44-9.35 (m, 2H), 8.84-8.72 (m, 2H), 8.23 (d, J=8.2 Hz, 1H), 8.14-8.05 (m, 2H), 8.03 (s, 1H), 7.95-7.89 (m, 1H), 7.69 (d, J=7.1 Hz, 1H), 7.45 (d, J=8.7 Hz, 1H), 7.28 (s, 1H), 7.21 (s, 1H), 6.24 (dd, J=12.6, 5.0 Hz, 1H), 4.43 (d, J=5.8 Hz, 2H), 3.79-3.72 (m, 4H), 3.39 (s, 3H), 3.00-2.83 (m, 2H), 2.78-2.70 (m, 1H), 2.42-2.35 (m, 1H), 1.34 (d, J=6.8 Hz, 6H).

### Example A4. N-(3-(1-(2,6-Dioxopiperidin-3-yl)-3-methoxy-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

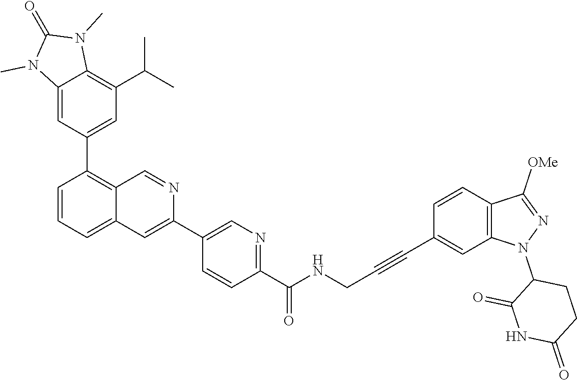 [SMILES: COc1nn(C2CCC(=O)NC2=O)c2cc(C#CCNC(=O)c3ccc(-c4cc5cccc(-c6cc(C(C)C)c7c(c6)n(C)c(=O)n7C)c5cn4)cn3)ccc12]

N-(3-(1-(2,6-Dioxopiperidin-3-yl)-3-methoxy-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-3-methoxy-1H-indazole according to the procedures described in Example A1, Steps A-D. The title compound (12 mg, 5% yield over 4 steps) was obtained as a white solid.

LC-MS (ESI): mass calcd. for C₄₃H₃₈N₈O₅, 746.3; m/z found, 747.2 [M+H]⁺.

¹H NMR (400 MHz, DMSO-d₆) δ 11.02 (s, 1H), 9.49 (d, J=1.7 Hz, 1H), 9.41-9.34 (m, 2H), 8.82-8.74 (m, 2H), 8.23 (d, J=8.3 Hz, 1H), 8.13-8.07 (m, 1H), 7.95-7.88 (m, 1H), 7.72-7.65 (m, 2H), 7.59 (d, J=8.3 Hz, 1H), 7.28 (s, 1H), 7.21 (d, J=1.2 Hz, 1H), 7.12-7.04 (m, 1H), 5.70 (dd, J=11.9, 5.1 Hz, 1H), 4.40 (d, J=5.9 Hz, 2H), 3.96 (s, 3H), 3.79-3.72 (m, 1H), 3.66 (s, 3H), 3.39 (s, 3H), 2.82-2.62 (m, 3H), 2.27-2.15 (m, 1H), 1.34 (d, J=6.8 Hz, 6H).

### Example A5. N-(3-(3-(2,6-Dioxopiperidin-3-yl)-1-methyl-1H-indazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: tert-Butyl 2-(5-bromo-1-methyl-1H-indazol-3-yl)-2-cyanoacetate

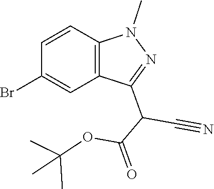 [SMILES: Cn1nc(C(C#N)C(=O)OC(C)(C)C)c2cc(Br)ccc21]

A solution of 5-bromo-3-iodo-1-methyl-1H-indazole (1.2 g, 3.5 mmol, 1.0 eq.) and tert-butyl 2-cyanoacetate (855 mg, 6.1 mmol, 1.7 eq.) in DMF (40 mL) was treated with CuI (67 mg, 356 μmol, 0.1 eq.), (1S,2S)-(+)-cyclohexane-1,2-diamine (81.3 mg, 712 μmol, 0.2 eq.) and Cs₂CO₃ (3.5 g, 10.7 mmol, 3.0 eq.) at room temperature. The mixture was stirred at 100° C. for 16 hours under N₂ atmosphere. The resulting solution was diluted with 80 mL of water and extracted with EtOAc (50 mL×3). The combined organic layers were washed with brine (40 mL), dried over Na₂SO₄, and concentrated under reduced pressure. The residue was purified by flash silica gel chromatography (100-200 mesh silica gel, 0-50% EtOAc in PE) to afford tert-butyl 2-(5-bromo-1-methyl-1H-indazol-3-yl)-2-cyanoacetate (800 mg, 64% yield) as a yellow oil.

LC-MS (ESI): mass calcd. for C₁₅H₁₆BrN₃O₂, 349.0/351.0; m/z found, 349.9/351.9 [M+H]⁺.

### Step B:]-(tert-Butyl) 5-ethyl 2-(5-bromo-1-methyl-1H-indazol-3-yl)-2-cyanopentanedioate

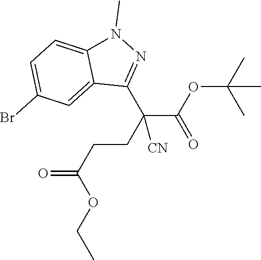 [SMILES: CCOC(=O)CCC(C#N)(C(=O)OC(C)(C)C)c1nn(C)c2ccc(Br)cc12]

A solution of tert-butyl 2-(5-bromo-1-methyl-1H-indazol-3-yl)-2-cyanoacetate (800 mg, 2.3 mmol, 1.0 eq.) in MeCN (15 mL) was treated with K₂CO₃ (631 mg, 4.6 mmol, 2.0 eq.) and benzyl(triethyl)ammonium chloride (52 mg, 228 μmol, 0.1 eq.) at room temperature. The mixture was stirred at 90° C. for 30 mins. At this point, ethyl 3-bromopropanoate (827 mg, 4.6 mmol, 2.0 eq.) was added to the solution. The mixture was then stirred at 90° C. for 16 hours. The resulting solution was diluted with 60 mL of water and extracted with EtOAc (50 mL×3). The combined organic layers were washed with brine (40 mL), dried over Na₂SO₄, and concentrated under reduced pressure. The residue was purified by flash silica gel chromatography (100-200 mesh silica gel, 0-50% EtOAc in PE) to give 1-(tert-butyl) 5-ethyl 2-(5-bromo-1-methyl-1H-indazol-3-yl)-2-cyanopentanedioate (500 mg, 49% yield) as a yellow oil.

LC-MS (ESI): mass calcd. for C₂₀H₂₄BrN₃O₄, 449.1/451.1; m/z found, 450.6/452.6 [M+H]⁺.

### Step C: 3-(5-Bromo-]-methyl-1H-indazol-3-yl)piperidine-2,6-dione

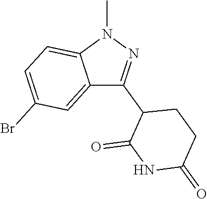 [SMILES: Cn1nc(C2CCC(=O)NC2=O)c2cc(Br)ccc21]

To a solution of 1-(tert-butyl) 5-ethyl 2-(5-bromo-1-methyl-1H-indazol-3-yl)-2-cyanopentanedioate (700 mg, 1.6 mmol, 1.0 eq.) in AcOH (16 mL) was added conc. H₂SO₄ (0.4 mL) at room temperature. The mixture was stirred at 120° C. for 2 hours. The reaction mixture was quenched by aq. NaHCO₃ (40 mL), and the mixture was diluted with water (30 mL) and extracted with DCM (40 mL×3). The combined organic layers were washed brine (40 mL), dried over Na₂SO₄, and concentrated under reduced pressure. The residue was purified by flash silica gel chromatography (100-200 mesh silica gel, 0-10% MeOH in DCM) to afford 3-(5-bromo-1-methyl-1H-indazol-3-yl)piperidine-2,6-dione (350 mg, 70% yield) as a white solid.

LC-MS (ESI): mass calcd. for C₁₃H₁₂BrN₃O₂, 321.0/323.0; m/z found, 321.9/323.9 [M+H]⁺.

### Step D: 3-(5-Bromo-1-methyl-1H-indazol-3-yl)-1-((2-(trimethylsilyl)ethoxy)methyl)piperidine-2,6-dione

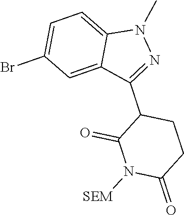 [SMILES: Cn1nc(C2CCC(=O)N(COCC[Si](C)(C)C)C2=O)c2cc(Br)ccc21]

A solution of 3-(5-bromo-1-methyl-1H-indazol-3-yl)piperidine-2,6-dione (200 mg, 621 μmol, 1.0 eq.) in DMF (8 mL) was treated with DBU (189 mg, 1.2 mmol, 2.0 eq.) at 0° C. Then, SEMCI (124 mg, 132 μL, 745 μmol, 1.2 eq.) was added at 0° C. The mixture was then stirred at room temperature for 16 hours. The resulting solution was diluted with 50 mL of water and extracted with EtOAc (50 mL×3). The combined organic layers were washed with brine (40 mL), dried over Na₂SO₄, and concentrated under reduced pressure. The residue was purified by flash silica gel chromatography (100-200 mesh silica gel, 0-50% EtOAc in PE) to afford 3-(5-bromo-1-methyl-1H-indazol-3-yl)-1-((2-(trimethylsilyl)ethoxy)methyl)piperidine-2,6-dione (180 mg, 64% yield) as an yellow oil.

LC-MS (ESI): mass calcd. for C₁₉H₂₆BrN₃O₃Si, 451.1/453.1; m/z found, 925.2/927.2/929.2 [2M+Na]+.

### Step E: tert-Butyl (3-(3-(2,6-dioxo-]-((2-(trimethylsilyl)ethoxy)methyl)piperidin-3-yl)-]-methyl-1H-indazol-5-yl)prop-2-yn-1-yl)carbamate

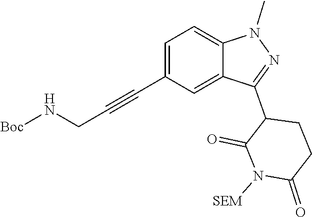 [SMILES: Cn1nc(C2CCC(=O)N(COCC[Si](C)(C)C)C2=O)c2cc(C#CCNC(=O)OC(C)(C)C)ccc21]

To a mixture of 3-(5-bromo-1-methyl-1H-indazol-3-yl)-1-((2-(trimethylsilyl)ethoxy)methyl)piperidine-2,6-dione (130 mg, 287 μmol, 1.0 eq.) and Pd(PPh₃)C₁₂ (20 mg, 28.7 μmol, 0.1 eq.) in DMF (8 mL) was added CuI (6 mg, 28.7 μmol, 0.1 eq.), tricyclohexylphosphine (20 mg, 71.8 μmol, 0.25 eq.), TEA (401 μL, 2.87 mmol, 10 eq.), and tert-butyl prop-2-yn-1-ylcarbamate (54 mg, 345 μmol, 1.2 eq.) at 25° C. The mixture was stirred at 85° C. for 16 hours under N₂ atmosphere. The reaction mixture was diluted with water (20 mL) and extracted with EtOAc (50 mL×3). The combined organic layers were washed brine (20 mL), dried over Na₂SO₄, and concentrated in vacuo. The residue was purified by flash silica gel chromatography (0-10% MeOH in DCM) to afford tert-butyl (3-(3-(2,6-dioxo-1-((2-(trimethylsilyl)ethoxy)methyl)piperidin-3-yl)-1-methyl-1H-indazol-5-yl)prop-2-yn-1-yl)carbamate (40 mg, 26% yield) as a yellow oil.

LC-MS (ESI): mass calcd. for C₂₇H₃₈N₄O₅Si, 526.3; m/z found, 549.2 [M+Na]+.

### Step F: 3-(5-(3-Aminoprop-1-yn-1-yl)-1-methyl-1H-indazol-3-yl)-1-(hydroxymethyl)piperidine-2,6-dione

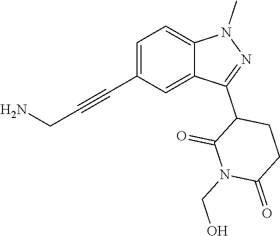 [SMILES: Cn1nc(C2CCC(=O)N(CO)C2=O)c2cc(C#CCN)ccc21]

To a solution of tert-butyl (3-(3-(2,6-dioxo-1-((2-(trimethylsilyl)ethoxy)methyl)piperidin-3-yl)-1-methyl-1H-indazol-5-yl)prop-2-yn-1-yl)carbamate (40 mg, 76 μmol, 1.0 eq.) in DCM (6 mL) was added TFA (1 mL) at 0° C. The reaction mixture was stirred at 25° C. for 2 hours. The reaction mixture was then concentrated in vacuo to give 3-(5-(3-aminoprop-1-yn-1-yl)-1-methyl-1H-indazol-3-yl)-1-(hydroxymethyl)piperidine-2,6-dione (40 mg, crude, TFA salt) as a yellow solid.

LC-MS (ESI): mass calcd. for C₁₇H₁₈N₄O₃, 326.1; m/z found, 327.4 [M+H]⁺.

### Step G: N-(3-(3-(]-(Hydroxymethyl)-2,6-dioxopiperidin-3-yl)-]-methyl-]H-indazol-5-yl)prop-2-yn-1-yl)-5-(8-(7-isopropyl-1,3-dimethyl-2-oxo-2,3-dihydro-]H-benzo[d]imidazol-5-yl)isoquinolin-3-yl)picolinamide

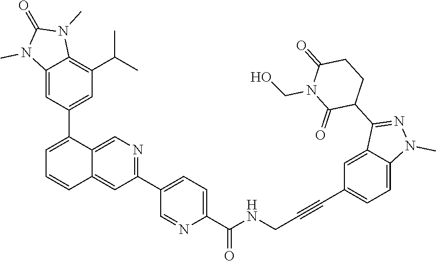

To a mixture of 5-(8-(7-isopropyl-1,3-dimethyl-2-oxo-2,3-dihydro-1H-benzo[d]imidazol-5-yl)isoquinolin-3-yl)picolinic acid (40 mg, 88.4 μmol, 1.0 eq.; Intermediate 2) in DMF (6 mL) was added 3-(5-(3-aminoprop-1-yn-1-yl)-1-methyl-1H-indazol-3-yl)-1-(hydroxymethyl)piperidine-2,6-dione (30 mg, 88.40 μmol, 1.0 eq.), HATU (67 mg, 176.8 μmol, 2.0 eq.), and DIEA (77 μL, 442.0 μmol, 5.0 eq.) at 0° C. The mixture was stirred at 25° C. for 2 hours. The reaction mixture was diluted with water (20 mL) and extracted with EtOAc (30 mL×3). The combined organic layers were washed brine (30 mL), dried over Na₂SO₄, and concentrated in vacuo. The residue was purified by flash silica gel chromatography (0-10% MeOH in DCM) to afford N-(3-(3-(1-(hydroxymethyl)-2,6-dioxopiperidin-3-yl)-1-methyl-1H-indazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide (45 mg, 67% yield) as a yellow solid.

LC-MS (ESI): mass calcd. for C₄₄H₄N₈O₅, 760.3; m/z found, 761.6 [M+H]⁺.

### Step H: N-(3-(3-(2,6-Dioxopiperidin-3-yl)-]-methyl-1H-indazol-5-yl)prop-2-yn-1-yl)-5-(8-(7-isopropyl-],3-dimethyl-2-oxo-2,3-dihydro-]H-benzo[d]imidazol-5-yl)isoquinolin-3-yl)picolinamide

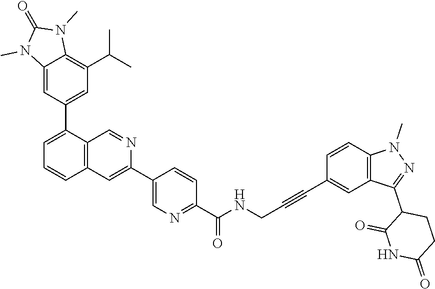 [SMILES: CC(C)c1cc(-c2cccc3cc(-c4ccc(C(=O)NCC#Cc5ccc6c(c5)c(C5CCC(=O)NC5=O)nn6C)nc4)ncc23)cc2c1n(C)c(=O)n2C]

To a solution of N-(3-(3-(1-(hydroxymethyl)-2,6-dioxopiperidin-3-yl)-1-methyl-1H-indazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide (45 mg, 59.1 μmol, 1.0 eq.) in MeCN (5 mL) was added NH₃·H₂O(0.1 mL) at 0° C. The reaction mixture was stirred at 25° C. for 20 mins. The resulting mixture was then treated with TFA until a sample was measured to be pH=4. The neutralized mixture was concentrated under reduced pressure. The residue was purified by Prep-HPLC (Triant C18 5 μm column, 0-60% MeCN in H₂O with 0.1% FA) to give N-(3-(3-(2,6-dioxopiperidin-3-yl)-1-methyl-1H-indazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide (2.2 mg, 5% yield) as a white solid.

LC-MS (ESI): mass calcd. for C₄₃H₃₈N₈O₄, 730.3; m/z found, 731.5 [M+H]⁺.

¹H NMR (400 MHz, DMSO-d₆) δ 10.88 (s, 1H), 9.51-9.48 (m, 1H), 9.40 (s, 1H), 9.36-9.31 (m, 1H), 8.82-8.76 (m, 2H), 8.23 (d, J=8.3 Hz, 1H), 8.12-8.08 (m, 1H), 7.94-7.87 (m, 2H), 7.70-7.67 (m, 1H), 7.63-7.59 (m, 1H), 7.44-7.40 (m, 1H), 7.30-7.27 (m, 1H), 7.22-7.19 (m, 1H), 4.42-4.36 (m, 3H), 4.00 (s, 3H), 3.79-3.72 (m, 1H), 3.66 (s, 3H), 3.39 (s, 3H), 2.68-2.59 (m, 2H), 2.40-2.32 (m, 1H), 2.20-2.08 (m, 1H), 1.35 (d, J=6.8 Hz, 6H).

### Example A6. N-(3-(1-(2,6-Dioxopiperidin-3-yl)-1H-pyrazolo[4,3-c]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

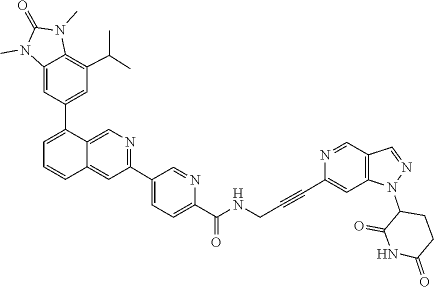 [SMILES: CC(C)c1cc(-c2cccc3cc(-c4ccc(C(=O)NCC#Cc5cc6c(cn5)cnn6C5CCC(=O)NC5=O)nc4)ncc23)cc2c1n(C)c(=O)n2C]

N-(3-(1-(2,6-Dioxopiperidin-3-yl)-1H-pyrazolo[4,3-c]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-1H-pyrazolo[4,3-c]pyridine according to the procedures described in Example A1, Steps A-D. The title compound (18 mg, 2% yield over 4 steps) was obtained as a white solid.

LC-MS (ESI): mass calcd. for C₄₁H₃₅N₉O₄, 717.3; m/z found, 718.3 [M+H]⁺.

¹H NMR (400 MHz, DMSO-d₆) δ 11.15 (s, 1H), 9.50 (d, J=1.7 Hz, 1H), 9.45-9.37 (m, 2H), 9.07 (s, 1H), 8.83-8.75 (m, 2H), 8.38 (s, 1H), 8.23 (d, J=8.2 Hz, 1H), 8.10 (d, J=8.3 Hz, 1H), 7.94-7.87 (m, 2H), 7.69 (d, J=7.0 Hz, 1H), 7.28 (d, J=1.3 Hz, 1H), 7.21 (d, 1H), 6.00-5.87 (m, 1H), 4.42 (d, J=5.9 Hz, 2H), 3.80-3.71 (m, 1H), 3.66 (s, 3H), 3.39 (s, 3H), 2.83-2.71 (m, 3H), 2.33-2.25 (m, 1H), 1.35 (d, J=6.8 Hz, 6H).

### Example A7. N-(3-(3-Cyano-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

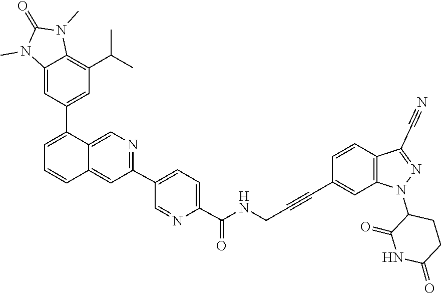 [SMILES: CC(C)c1cc(-c2cccc3cc(-c4ccc(C(=O)NCC#Cc5ccc6c(C#N)nn(C7CCC(=O)NC7=O)c6c5)nc4)ncc23)cc2c1n(C)c(=O)n2C]

N-(3-(3-Cyano-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-1H-indazole-3-carbonitrile according to the procedures described in Example A1, Steps A-D. The title compound (16 mg, 2% yield over 4 steps) was obtained as a white solid.

LC-MS (ESI): mass calcd. for C₄₃H₃₅N₉O₄, 741.3; m/z found, 742.3 [M+H]⁺.

¹H NMR (400 MHz, DMSO-d₆) δ 11.20 (s, 1H), 9.50 (d, J=1.7 Hz, 1H), 9.44-9.37 (m, 2H), 8.83-8.78 (m, 1H), 8.77 (s, 1H), 8.23 (d, J=8.3 Hz, 1H), 8.13-8.06 (m, 2H), 7.96-7.87 (m, 2H), 7.69 (d, J=7.1 Hz, 1H), 7.45 (d, J=8.5 Hz, 1H), 7.28 (d, J=1.3 Hz, 1H), 7.21 (d, J=1.2 Hz, 1H), 6.20-6.02 (m, 1H), 4.43 (d, J=5.9 Hz, 2H), 3.79-3.71 (m, 1H), 3.66 (s, 3H), 3.39 (s, 3H), 2.84-2.69 (m, 3H), 2.38-2.30 (m, 1H), 1.35 (d, J=6.8 Hz, 6H).

### Example A8. N-(3-(1-(2,6-Dioxopiperidin-3-yl)-5-methyl-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

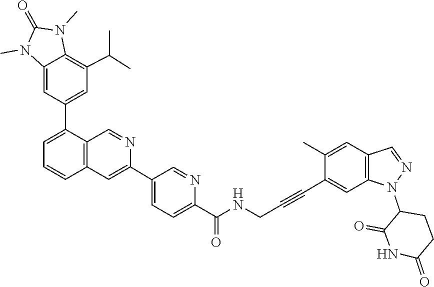 [SMILES: Cc1cc2cnn(C3CCC(=O)NC3=O)c2cc1C#CCNC(=O)c1ccc(-c2cc3cccc(-c4cc(C(C)C)c5c(c4)n(C)c(=O)n5C)c3cn2)cn1]

N-(3-(1-(2,6-Dioxopiperidin-3-yl)-5-methyl-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-5-methyl-1H-indazole according to the procedures described in Example A1, Steps A D. The title compound (10 mg, 3% yield over 4 steps) was obtained as a white solid.

LC-MS (ESI): mass calcd. for C₄₃H₃₈N₈O₄, 730.3; m/z found, 731.4 [M+H]⁺.

¹H NMR (400 MHz, DMSO-d₆) δ 11.07 (s, 1H), 9.50 (d, J=2.0 Hz, 1H), 9.42-9.36 (m, 2H), 8.82-8.77 (m, 1H), 8.76 (s, 1H), 8.23 (d, J=8.2 Hz, 1H), 8.10 (d, J=8.2 Hz, 1H), 8.05 (s, 1H), 7.91 (t, J=7.7 Hz, 1H), 7.78 (s, 1H), 7.68 (d, J=7.1 Hz, 1H), 7.64 (s, 1H), 7.28 (s, 1H), 7.21 (s, 1H), 5.91-5.81 (m, 1H), 4.45 (d, J=5.9 Hz, 2H), 3.78-3.71 (m, 1H), 3.66 (s, 3H), 3.39 (s, 3H), 2.82-2.66 (m, 3H), 2.46 (s, 3H), 2.27-2.20 (m, 1H), 1.34 (d, J=6.8 Hz, 6H).

### Example A9. N-(3-(4-(Dimethylamino)-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### StepA: 4-Bromo-2-(dimethylamino)-6-fluorobenzaldehyde

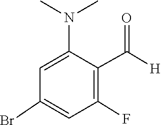

To a stirred mixture of 4-bromo-2,6-difluorobenzaldehyde (10 g, 45.2 mmol, 1 eq.) and K₂CO₃ (13.8 g, 99.5 mmol, 2.2 eq.) in DMF (60 mL) was added Me2NH—HCl (4 g, 49.8 mmol, 1.1 eq.) at room temperature. The reaction mixture was stirred at 50° C. for 16 hours under N₂ atmosphere. The mixture was diluted with water and extracted with EtOAc (100 mL×3). The combined organic layers were washed with brine (50 mL), dried over anhydrous Na₂SO₄, and concentrated under reduced pressure. The residue was purified by flash column chromatography on silica gel (100-200 mesh silica gel, 0-40% EtOAc in PE) to give 4-bromo-2-(dimethylamino)-6-fluorobenzaldehyde (9.5 g, 85% yield) as a yellow oil.

LC-MS (ESI): mass calcd. for C₉H₉BrFNO, 245.0/247.0; m/z found, 246/248 [M+H]⁺.

### Step B: 6-bromo-N,N-dimethyl-1H-indazol-4-amine

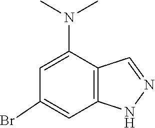

A sample of 4-bromo-2-(dimethylamino)-6-fluorobenzaldehyde (500 mg, 2.03 mmol, 1 eq.) in a mixture of hydrazine hydrate (5 mL) and DME (5 mL) was placed under N₂ atmosphere and stirred at 180° C. for 7 hours. The mixture was diluted with 1M aq. HCl (60 mL) and extracted with EtOAc (100 mL×3). The combined organic layers were washed with brine (50 mL), dried over anhydrous Na₂SO₄, and concentrated under reduced pressure. The residue was purified by flash column chromatography on silica gel (100-200 mesh silica gel, 0-40% EtOAc in PE) to give 6-bromo-N,N-dimethyl-1H-indazol-4-amine (105 mg, 22% yield) as a white solid.

LC-MS (ESI): mass calcd. for C₉H₁₀BrN₃, 239.0/241.0; m/z found, 240/242 [M+H]⁺.

Steps C—F: N-(3-(4-(Dimethylamino)-]-(2,6-dioxopiperidin-3-yl)-]H-indazol-6-yl)prop-2-yn-]-yl)-5-(8-(7-isopropyl-],3-dimethyl-2-oxo-2,3-dihydro-]H-benzo[d]imidazol-5-yl)isoquinolin-3-yl)picolinamide

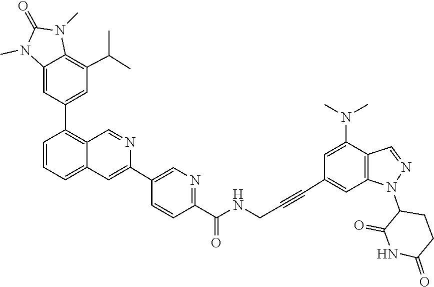 [SMILES: CC(C)c1cc(-c2cccc3cc(-c4ccc(C(=O)NCC#Cc5cc(N(C)C)c6cnn(C7CCC(=O)NC7=O)c6c5)nc4)ncc23)cc2c1n(C)c(=O)n2C]

N-(3-(4-(Dimethylamino)-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-N,N-dimethyl-1H-indazol-4-amine according to the procedures described in Example A1, Steps A-D. The title compound (11 mg, 10% yield over 4 steps) was obtained as a white solid.

LC-MS (ESI): mass calcd. for C₄₄H₄₁N₉O₄, 759.3; m/z found, 760.3 [M+H]⁺.

¹H NMR (400 MHz, DMSO-d₆) δ 11.06 (s, 1H), 9.50 (d, J=2.1 Hz, 1H), 9.40 (s, 1H), 9.34 (t, J=6.0 Hz, 1H), 8.82-8.75 (m, 2H), 8.26-8.20 (m, 2H), 8.10 (d, J=8.3 Hz, 1H), 7.92 (t, J=7.7 Hz, 1H), 7.68 (dd, J=7.1, 1.1 Hz, 1H), 7.28 (d, J=1.6 Hz, 1H), 7.21 (d, J=1.7 Hz, 1H), 7.07 (s, 1H), 6.18 (s, 1H), 5.77 (dd, J=11.6, 5.0 Hz, 1H), 4.38 (d, J=6.0 Hz, 2H), 3.79-3.71 (m, 1H), 3.66 (s, 3H), 3.39 (s, 3H), 3.12 (s, 6H), 2.85-2.76 (m, 1H), 2.73-2.65 (m, 2H), 2.23-2.16 (m, 1H), 1.35 (d, J=6.7 Hz, 6H).

### Example A10. N-(3-(1-(2,6-Dioxopiperidin-3-yl)isoquinolin-7-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: 7-Bromoisoquinoline 2-oxide

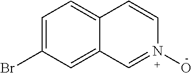

To a solution of 7-bromoisoquinoline (2.3 g, 11.1 mmol, 1 eq.) in DCM (55 mL) was added 3-chloroperoxybenzoic acid (2.9 g, 16.6 mmol, 1.5 eq.) at 0° C. The mixture was stirred at 25° C. for 16 hours under N₂ atmosphere. The reaction mixture was quenched with sat. aq. Na₂S₂O₃ (100 mL), diluted with 100 mL of aq. NaHCO₃, and extracted with DCM (100 mL×3). The combined organic layers were washed with aq. NaHCO₃ and brine, dried over Na₂SO₄, and concentrated under reduced pressure. The residue was purified by flash column chromatography on silica gel (100-200 mesh silica gel, 0-50% EtOAc in PE) to give 7-bromoisoquinoline 2-oxide (1.9 g, 77% yield) as a white solid.

LC-MS (ESI): mass calcd. for C₉H₆BrNO, 223.0/225.0; m/z found, 224/226 [M+H]⁺.

¹H NMR (400 MHz, DMSO-d₆) δ 8.91 (s, 1H), 8.24-8.13 (m, 2H), 7.94 (dd, J=17.8, 7.8 Hz, 2H), 7.77-7.68 (m, 1H).

### Step B: Methyl 2-(7-bromoisoquinolin-1-yl)acetate

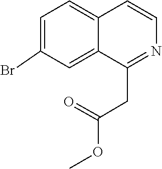 [SMILES: COC(=O)Cc1nccc2ccc(Br)cc12]

To a solution of 7-bromoisoquinoline 2-oxide (900 mg, 4.02 mmol, 1 eq.) in THE (20 mL) was added PyBop (2.1 g, 4.42 mmol, 1.1 eq.), tert-butyl((1-methoxyvinyl)oxy)dimethylsilane (1.5 g, 8.03 mmol, 2 eq.) and DIPEA (1.6 g, 12.1 mmol, 3 eq.) at 0° C. The mixture was stirred at 25° C. for 10 mins under N₂ atmosphere. The resulting solution was diluted with 30 mL of H₂O and extracted with EtOAc (30 mL×3). The combined organic layers were dried over anhydrous sodium sulfate and concentrated under reduced pressure. The residue was purified by flash column chromatography on silica gel (100-200 mesh silica gel, 0-25% EtOAc in PE) to give methyl 2-(7-bromoisoquinolin-1-yl)acetate (248 mg, 22% yield) as a yellow oil.

LC-MS (ESI): mass calcd. for C₁₂H₁₀BrNO₂, 279.0/281.0; m/z found, m/z 280/282 [M+H]⁺.

### Step C: 3-(7-Bromoisoquinolin-1-yl)piperidine-2,6-dione

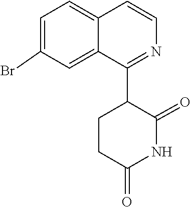 [SMILES: O=C1CCC(c2nccc3ccc(Br)cc23)C(=O)N1]

To a solution of methyl 2-(7-bromoisoquinolin-1-yl)acetate (240 mg, 857 μmol, 1 eq.) in DMF (10 mL) was added potassium tert-butoxide (96 mg, 857 μmol, 1 eq.) and acrylamide (244 mg, 3.43 mmol, 4 eq.) at 0° C. The mixture was stirred at 25° C. for 2 hours under N₂ atmosphere. The resulting solution was diluted with 30 mL of H₂O, extracted with EtOAc (30 mL×3), and washed with brine (30 mL). The combined organic layers were dried over anhydrous sodium sulfate and concentrated in vacuo. The residue was purified by flash column chromatography on silica gel (100-200 mesh silica gel, 0-30% EtOAc in PE) to give 3-(7-bromoisoquinolin-1-yl)piperidine-2,6-dione (78 mg, 29% yield) as a yellow solid.

LC-MS (ESI): mass calcd. for C₁₄H₁₁BrN₂O₂, 318.0/320.0; m/z found, 319/321 [M+H]⁺.

### Steps D F: N-(3-(1-(2,6-Dioxopiperidin-3-yl)isoquinolin-7-yl)prop-2-yn-1-yl)-5-(8-(7-isopropyl-1,3-dimethyl-2-oxo-2,3-dihydro-]H-benzo[d]imidazol-5-yl)isoquinolin-3-yl)picolinamide

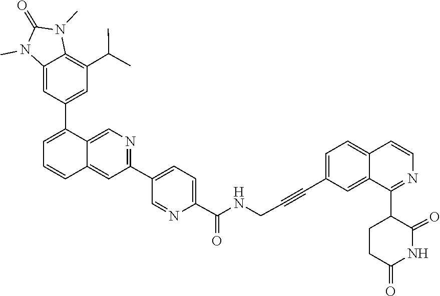 [SMILES: CC(C)c1cc(-c2cccc3cc(-c4ccc(C(=O)NCC#Cc5ccc6ccnc(C7CCC(=O)NC7=O)c6c5)nc4)ncc23)cc2c1n(C)c(=O)n2C]

### N-(3-(1-(2,6-Dioxopiperidin-3-yl)isoquinolin-7-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

was prepared from 3-(7-bromoisoquinolin-1-yl)piperidine-2,6-dione according to the procedures described in Example A1, Steps B D. The title compound (11 mg, 21% yield over 3 steps) was obtained as a white solid.

LC-MS (ESI): mass calcd. for C₄₄H₃₇N₇O₄, 727.3; m/z found, 728.3 [M+H]⁺.

¹H NMR (400 MHz, DMSO-d₆) δ 10.89 (s, 1H), 9.50 (d, J=1.8 Hz, 1H), 9.46-9.37 (m, 2H), 8.84-8.72 (m, 2H), 8.49-8.38 (m, 2H), 8.24 (d, J=8.3 Hz, 1H), 8.10 (d, J=8.3 Hz, 1H), 8.00 (d, J=8.6 Hz, 1H), 7.95-7.87 (m, 1H), 7.82-7.74 (m, 2H), 7.69 (d, J=7.1 Hz, 1H), 7.28 (s, 1H), 7.21 (d, J=1.2 Hz, 1H), 5.10 (dd, J=9.3, 5.1 Hz, 1H), 4.46 (d, J=5.9 Hz, 2H), 3.82-3.70 (m, 1H), 3.66 (s, 3H), 3.39 (s, 3H), 2.73-2.63 (m, 1H), 2.59-2.52 (m, 1H), 2.48-2.40 (m, 1H), 2.34-2.10 (m, 1H), 1.35 (d, J=6.8 Hz, 6H).

### Example A11. N-(3-(1-(2,6-Dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

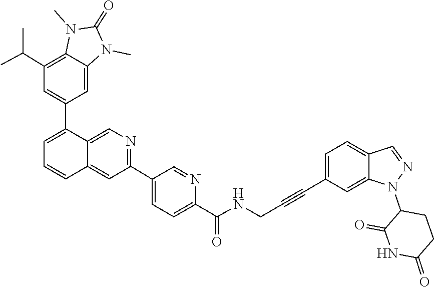 [SMILES: CC(C)c1cc(-c2cccc3cc(-c4ccc(C(=O)NCC#Cc5ccc6cnn(C7CCC(=O)NC7=O)c6c5)nc4)ncc23)cc2c1n(C)c(=O)n2C]

N-(3-(1-(2,6-Dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-1H-indazole according to the procedures described in Example A1, Steps A D. The title compound (9.0 mg, 7% yield over 4 steps) was obtained as a white solid.

LC-MS (ESI): mass calcd. for C₄₂H₃₆N₈O₄, 716.3; m/z found, 717.3 [M+H]⁺.

¹H NMR (400 MHz, DMSO-d₆) δ 11.08 (s, 1H), 9.50 (d, J=1.4 Hz, 1H), 9.40 (s, 1H), 9.37 (d, J=6.1 Hz, 1H), 8.80 (d, J=8.3 Hz, 1H), 8.77 (s, 1H), 8.23 (d, J=8.2 Hz, 1H), 8.15 (s, 1H), 8.10 (d, J=8.0 Hz, 1H), 7.92 (t, J=7.6 Hz, 1H), 7.82 (s, 1H), 7.78 (d, J=8.4 Hz, 1H), 7.69 (d, J=7.0 Hz, 1H), 7.28 (s, 1H), 7.21-7.17 (m, 2H), 5.88 (dd, J=7.4, 4.2 Hz, 1H), 4.41 (d, J=5.7 Hz, 2H), 3.78-3.72 (m, 1H), 3.66 (s, 3H), 3.39 (s, 3H), 2.79-2.66 (m, 3H), 2.28-2.22 (m, 1H), 1.35 (d, J=6.8 Hz, 6H).

### Example A12. N-(3-(1-(2,6-Dioxopiperidin-3-yl)-1H-pyrazolo[3,4-b]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

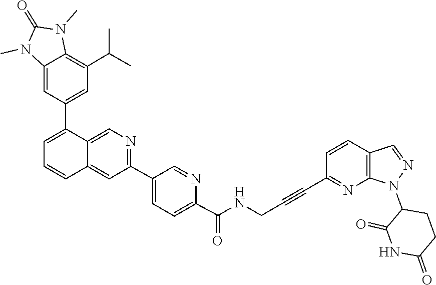 [SMILES: CC(C)c1cc(-c2cccc3cc(-c4ccc(C(=O)NCC#Cc5ccc6cnn(C7CCC(=O)NC7=O)c6n5)nc4)ncc23)cc2c1n(C)c(=O)n2C]

N-(3-(1-(2,6-Dioxopiperidin-3-yl)-1H-pyrazolo[3,4-b]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-1H-pyrazolo[3,4-b]pyridine according to the procedures described in Example A1, Steps A-D. The title compound (23 mg, 9% yield over 4 steps) was obtained as a white solid.

LC-MS (ESI): mass calcd. for C₄₁H₃₅N₉O₄, 717.3; m/z found, 718.5 [M+H]⁺.

¹H NMR (400 MHz, DMSO-d₆) δ 11.08 (s, 1H), 9.50 (s, 1H), 9.45 (t, J=6.0 Hz, 1H), 9.40 (s, 1H), 8.83-8.74 (m, 2H), 8.30 (d, J=8.2 Hz, 1H), 8.27-8.20 (m, 2H), 8.10 (d, J=8.3 Hz, 1H), 7.92 (t, J=7.7 Hz, 1H), 7.69 (d, J=7.1 Hz, 1H), 7.38 (d, J=8.2 Hz, 1H), 7.28 (s, 1H), 7.21 (s, 1H), 5.99 (dd, J=12.4, 5.2 Hz, 1H), 4.44 (d, J=5.9 Hz, 2H), 3.80-3.71 (m, 1H), 3.66 (s, 3H), 3.39 (s, 3H), 3.05-2.94 (m, 1H), 2.82-2.64 (m, 2H), 2.30-2.19 (m, 1H), 1.35 (d, J=6.8 Hz, 6H).

### Example A13.N-(3-(1-(2,6-Dioxopiperidin-3-yl)-1H-pyrrolo[3,2-b]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

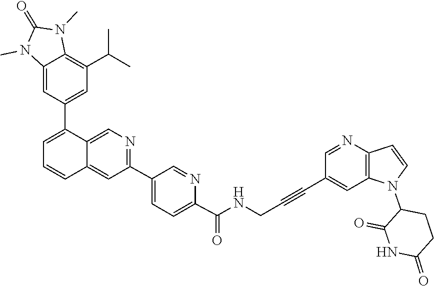 [SMILES: CC(C)c1cc(-c2cccc3cc(-c4ccc(C(=O)NCC#Cc5cnc6ccn(C7CCC(=O)NC7=O)c6c5)nc4)ncc23)cc2c1n(C)c(=O)n2C]

N-(3-(1-(2,6-Dioxopiperidin-3-yl)-1H-pyrrolo[3,2-b]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-1H-pyrrolo[3,2-b]pyridine according to the procedures described in Example A1, Steps A-D. The title compound (2.2 mg, 14% yield over 4 steps) was obtained as a white solid.

LC-MS (ESI): mass calcd. for C₄₂H₃₆N₈O₄, 716.3; m/z found, 717.4 [M+H]⁺.

¹H NMR (400 MHz, DMSO-d₆) δ 11.10 (s, 1H), 9.50 (d, J=1.7 Hz, 1H), 9.41-9.35 (m, 2H), 8.80 (dd, J=8.2, 2.1 Hz, 1H), 8.77 (s, 1H), 8.39 (s, 1H), 8.23 (d, J=8.2 Hz, 1H), 8.12-8.08 (m, 1H), 8.06 (s, 1H), 7.94-7.89 (m, 1H), 7.79 (d, J=3.3 Hz, 1H), 7.68 (d, J=7.1 Hz, 1H), 7.30-7.26 (m, 1H), 7.21 (d, 1H), 6.66 (d, J=3.2 Hz, 1H), 5.73-5.65 (m, 1H), 4.42 (d, J=5.9 Hz, 2H), 3.80-3.72 (m, 1H), 3.66 (s, 3H), 3.39 (s, 3H), 2.84-2.65 (m, 3H), 2.19-2.12 (m, 1H), 1.35 (d, 6H).

### Example A14. N-(3-(3-(2,6-Dioxopiperidin-3-yl)-2-oxo-2,3-dihydrobenzo[d]oxazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: 3-(5-Bromo-2-oxobenzo[d]oxazol-3(2H)-yl)piperidine-2,6-dione

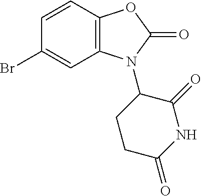 [SMILES: O=C1CCC(n2c(=O)oc3ccc(Br)cc32)C(=O)N1]

To a solution of 5-bromobenzo[d]oxazol-2(3H)-one (300 mg, 1.4 mmol, 1.0 eq.) in anhydrous DMF (10 mL) was added NaH (34 mg, 1.4 mmol, 1.0 eq.) at 0° C. After 30 min, 3-bromopiperidine-2,6-dione (269 mg, 1.4 mmol, 1.0 eq.) was added. The mixture was stirred for 2 hours at room temperature under N₂ atmosphere. The reaction mixture was quenched with 50 mL of H₂O and extracted with EtOAc (40 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give 3-(5-bromo-2-oxobenzo[d]oxazol-3(2H)-yl)piperidine-2,6-dione (100 mg, 33% yield) as a brown solid.

LC-MS (ESI): mass calcd. for C₁₂H₉BrN₂O₄, 324.0/326.0; m/z found, 325/327 [M+H]⁺.

### Steps B-D: N-(3-(3-(2,6-Dioxopiperidin-3-yl)-2-oxo-2,3-dihydrobenzo[d]oxazol-5-yl)prop-2-yn-1-yl)-5-(8-(7-isopropyl-],3-dimethyl-2-oxo-2,3-dihydro-]H-benzo[d]imidazol-5-yl)isoquinolin-3-yl)picolinamide

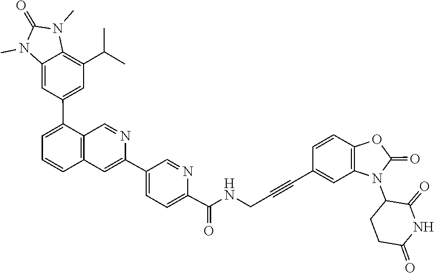 [SMILES: CC(C)c1cc(-c2cccc3cc(-c4ccc(C(=O)NCC#Cc5ccc6oc(=O)n(C7CCC(=O)NC7=O)c6c5)nc4)ncc23)cc2c1n(C)c(=O)n2C]

N-(3-(3-(2,6-Dioxopiperidin-3-yl)-2-oxo-2,3-dihydrobenzo[d]oxazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 3-(5-bromo-2-oxobenzo[d]oxazol-3(2H)-yl)piperidine-2,6-dione according to the procedures described in Example A1, Steps B D. The title compound (11.8 mg, 16% yield over 3 steps) was obtained as a white solid.

LC-MS (ESI): mass calcd. for C₄₂H₃₅N₇O₆, 733.3; m/z found, 734.6 [M+H]⁺.

¹H NMR (400 MHz, DMSO-d₆) δ 11.17 (s, 1H), 9.49 (s, 1H), 9.40 (s, 1H), 9.34 (t, J=5.9 Hz, 1H), 8.83-8.75 (m, 2H), 8.22 (d, J=8.2 Hz, 1H), 8.10 (d, J=8.5 Hz, 1H), 7.92 (t, J=7.5 Hz, 1H), 7.69 (d, J=7.1 Hz, 1H), 7.44-7.37 (m, 2H), 7.29-7.23 (m, 2H), 7.21 (s, 1H), 5.38 (dd, J=12.6, 5.5 Hz, 1H), 4.37 (d, J=5.9 Hz, 2H), 3.80-3.72 (m, 1H), 3.66 (s, 3H), 3.39 (s, 3H), 2.88-2.79 (m, 1H), 2.72-2.60 (m, 2H), 2.18-2.06 (m, 1H), 1.34 (d, J=6.7 Hz, 6H).

### Example A15. N-(3-(1-(2,6-Dioxopiperidin-3-yl)-1H-benzo[d]imidazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: 3-(6-Bromo-]H-benzo[d]imidazol-1-yl)piperidine-2,6-dione and 3-(5-bromo-1H-benzo[d]imidazol-1-yl)piperidine-2,6-dione

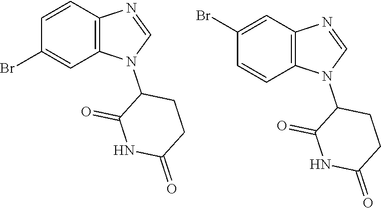 [SMILES: O=C1CCC(n2cnc3ccc(Br)cc32)C(=O)N1 ; O=C1CCC(n2cnc3cc(Br)ccc32)C(=O)N1]

To a mixture of 6-bromo-1H-benzo[d]imidazole (1 g, 5.1 mmol, 1.0 eq.) and 3-bromopiperidine-2,6-dione (1.5 g, 7.6 mmol, 1.5 eq.) in THF (40 mL) was added t-BuOK (1.7 g, 15.2 mmol, 3.0 eq.) at room temperature. The mixture was stirred at 50° C. under N₂ for 16 hours. The resulting solution was diluted with 60 mL of H₂O and extracted with EtOAc (40 mL×3). The combined organic layers were washed with brine, dried over Na₂SO₄, and concentrated under reduced pressure. The residue was purified by flash chromatography (100-200 mesh silica gel, 0-70% EtOAc in PE) to afford a mixture (400 mg, 23% combined yield) of 3-(6-bromo-1H-benzo[d]imidazol-1-yl)piperidine-2,6-dione and 3-(5-bromo-1H-benzo[d]imidazol-1-yl)piperidine-2,6-dione as a yellow solid.

LC-MS (ESI): mass calcd. for C₁₂H₁₀BrN₃O₂, 307.0/309.0; m/z found, 308.1/310.1 [M+H]⁺.

### Step B: tert-Butyl (3-(1-(2,6-dioxopiperidin-3-yl)-]H-benzo[d]imidazol-6-yl)prop-2-yn-1-yl)carbamate andtert-butyl (3-(1-(2,6-dioxopiperidin-3-yl)-H-benzo[d]imidazol-5-yl)prop-2-yn-I-yl)carbamate

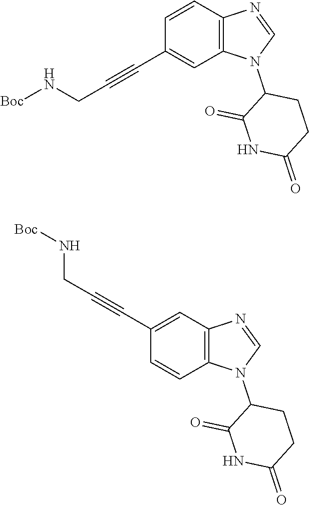 [SMILES: CC(C)(C)OC(=O)NCC#Cc1ccc2ncn(C3CCC(=O)NC3=O)c2c1 ; CC(C)(C)OC(=O)NCC#Cc1ccc2c(c1)ncn2C1CCC(=O)NC1=O]

To a mixture (290 mg, 941.1 μmol, 1.0 eq.) of 3-(6-bromo-1H-benzo[d]imidazol-1-yl)piperidine-2,6-dione and 3-(5-bromo-1H-benzo[d]imidazol-1-yl)piperidine-2,6-dione in DMF (20 mL) was added Pd(PPh₃)Cl₂ (132 mg, 188.2 μmol, 0.2 eq.), tert-butyl prop-2-yn-1-ylcarbamate (438 mg, 2.8 mmol, 3.0 eq.), CuI (36 mg, 188.2 μmol, 0.2 eq.), and TEA (1.3 mL, 9.4 mmol, 10.0 eq.) at room temperature. The mixture was stirred at 100° C. under N₂ for 3 hours. The resulting solution was diluted with 50 mL of H₂O and extracted with EtOAc (40 mL×3). The combined organic layers were washed with brine, dried over Na₂SO₄, and concentrated under reduced pressure. The residue was purified by flash chromatography (100-200 mesh silica gel, 0-80% EtOAc in PE) to give a mixture (130 mg, 36% combined yield) of tert-butyl (3-(1-(2,6-dioxopiperidin-3-yl)-1H-benzo[d]imidazol-6-yl)prop-2-yn-1-yl)carbamate and tert-butyl (3-(1-(2,6-dioxopiperidin-3-yl)-1H-benzo[d]imidazol-5-yl)prop-2-yn-1-yl)carbamate as a yellow solid.

LC-MS (ESI): mass calcd. for C₂₀H₂₂N₄O₄, 382.2; m/z found, 383.1 [M+H]⁺.

### Step C: 3-(6-(3-Aminoprop-1-yn-1-yl)-]H-benzo[d]imidazol-1-yl)piperidine-2,6-dione and 3-(5-(3-aminoprop-1-yn-1-yl)-]H-benzo[d]imidazol-1-yl)piperidine-2,6-dione

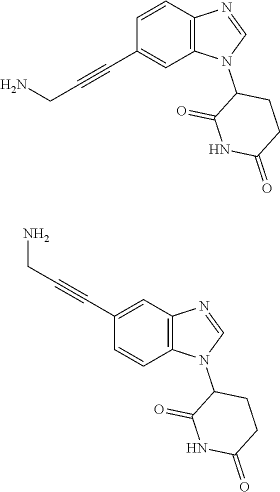 [SMILES: NCC#Cc1ccc2ncn(C3CCC(=O)NC3=O)c2c1 ; NCC#Cc1ccc2c(c1)ncn2C1CCC(=O)NC1=O]

To a mixture (100 mg, 261.5 μmol, 1.0 eq.) of tert-butyl (3-(1-(2,6-dioxopiperidin-3-yl)-1H-benzo[d]imidazol-6-yl)prop-2-yn-1-yl)carbamate and tert-butyl (3-(1-(2,6-dioxopiperidin-3-yl)-1H-benzo[d]imidazol-5-yl)prop-2-yn-1-yl)carbamate in DCM (4.5 mL) was added HCl (1.5 mL, 4M in 1,4-dioxane) at room temperature. The mixture was stirred at room temperature under N₂ for 1 hour. The mixture was concentrated under reduced pressure to give a mixture of (100 mg, crude, HCl salt) of 3-(6-(3-aminoprop-1-yn-1-yl)-1H-benzo[d]imidazol-1-yl)piperidine-2,6-dione and 3-(5-(3-aminoprop-1-yn-1-yl)-1H-benzo[d]imidazol-1-yl)piperidine-2,6-dione as a pink solid.

LC-MS (ESI): mass calcd. for C₁₅H₁₄N₄O₂, 282.1; m/z found, 283.1 [M+H]⁺.

### Step D: N-(3-(1-(2,6-Dioxopiperidin-3-yl)-]H-benzo[d]imidazol-6-yl)prop-2-yn-1-yl)-5-(8-(7-isopropyl-4,3-dimethyl-2-oxo-2,3-dihydro-]H-benzo[d]imidazol-5-yl)isoquinolin-3-yl)picolinamide

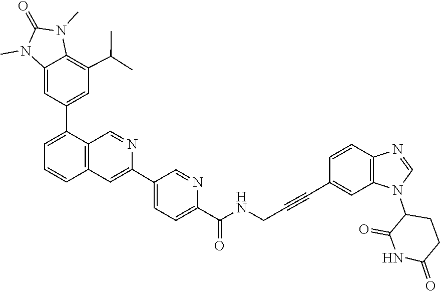 [SMILES: CC(C)c1cc(-c2cccc3cc(-c4ccc(C(=O)NCC#Cc5ccc6ncn(C7CCC(=O)NC7=O)c6c5)nc4)ncc23)cc2c1n(C)c(=O)n2C]

A mixture (50 mg, 177 μmol, 2.0 eq.) of 3-(6-(3-aminoprop-1-yn-1-yl)-1H-benzo[d]imidazol-1-yl)piperidine-2,6-dione and 3-(5-(3-aminoprop-1-yn-1-yl)-1H-benzo[d]imidazol-1-yl)piperidine-2,6-dione was dissolved in DMF (3 mL) and treated with 5-(8-(7-isopropyl-1,3-dimethyl-2-oxo-2,3-dihydro-1H-benzo[d]imidazol-5-yl)isoquinolin-3-yl)picolinic acid (40 mg, 88.4 μmol, 1.0 eq.; Intermediate 2), HATU (44 mg, 115 μmol, 1.3 eq.), and DIPEA (46 μL, 265 μmol, 3.0 eq.) at 0° C. The mixture was stirred at room temperature for 1 hour under N₂ atmosphere. The resulting solution was diluted with 20 mL of H₂O and extracted with EtOAc (20 mL×3). The combined organic layers were washed with brine, dried over Na₂SO₄, and concentrated under reduced pressure. The resulting solution was purified by Prep-HPLC (Triant C18 5 μm column, 0-60% MeOH in H₂O with 0.1% FA) to afford N-(3-(1-(2,6-dioxopiperidin-3-yl)-1H-benzo[d]imidazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide (6.4 mg, 10% yield) as a white solid.

LC-MS (ESI): mass calcd. for C₄₂H₃₆N₈O₄, 716.3; m/z found, 717.5 [M+H]⁺.

¹H NMR (400 MHz, DMSO-d₆) δ 11.25-11.19 (m, 1H), 9.49 (d, J=1.6 Hz, 1H), 9.40 (s, 1H), 9.36 (t, J=5.9 Hz, 1H), 8.80 (dd, J=8.2, 2.2 Hz, 1H), 8.77 (s, 1H), 8.66-8.55 (m, 1H), 8.23 (d, J=8.2 Hz, 1H), 8.10 (d, J=8.3 Hz, 1H), 7.95-7.89 (m, 1H), 7.82 (s, 1H), 7.72-7.67 (m, 2H), 7.39-7.31 (m, 1H), 7.28 (d, J=1.5 Hz, 1H), 7.21 (d, J=1.4 Hz, 1H), 5.82-5.74 (m, 1H), 4.40 (d, J=6.0 Hz, 2H), 3.77-3.73 (m, 1H), 3.66 (s, 3H), 3.39 (s, 3H), 2.87-2.78 (m, 2H), 2.73-2.67 (m, 1H), 2.30-2.22 (m, 1H), 1.35 (d, J=6.8 Hz, 6H).

### Example A16. (E)-N-(3-(3-((2,6-Dioxopiperidin-3-yl)amino)-1-methyl-1H-pyrazol-5-yl)allyl)-5-(8-(7-isopropyl-1,3-dimethyl-2-oxo-2,3-dihydro-1H-benzo[d]imidazol-5-yl)isoquinolin-3-yl)picolinamide

### Step A: 3-((5-Bromo-1-methyl-1H-pyrazol-3-yl)amino)piperidine-2,6-dione

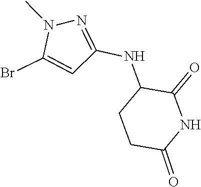 [SMILES: Cn1nc(NC2CCC(=O)NC2=O)cc1Br]

A solution of 5-bromo-1-methyl-1H-pyrazol-3-amine (100 mg, 568 μmol, 1.0 eq.) and DIPEA (0.2 mL, 1.1 mmol, 2.0 eq.) in 1,4-dioxane (0.25 mL) was treated with 3-bromopiperidine-2,6-dione (218 mg, 1.1 mmol, 2.0 eq.) at room temperature. The mixture was then stirred for 16 hours at 80° C. The resulting solution was diluted with 50 mL of water and extracted with EtOAc (30 mL×3). The combined organic layers were washed with brine (50 mL), dried over Na₂SO₄, and concentrated under reduced pressure. The residue was purified by flash column chromatography on silica gel (100-200 mesh silica gel, 0-50% EtOAc in PE) to give 3-((5-bromo-1-methyl-1H-pyrazol-3-yl)amino)piperidine-2,6-dione (80 mg, 49% yield) as a blue solid.

LC-MS (ESI): mass calcd. for C₉H₁₁BrN₄O₂, 286.0/288.0; m/z found, 287.1/289.1 [M+H]⁺.

¹H NMR (400 MHz, DMSO-d₆) δ 10.76 (s, 1H), 5.76-5.71 (m, 2H), 4.24-4.12 (m, 1H), 3.59 (s, 3H), 2.75-2.65 (m, 1H), 2.55 (t, J=3.9 Hz, 1H), 2.21-2.13 (m, 1H), 1.94-1.83 (m, 1H).

### Step B: tert-Butyl (E)-(3-(3-((2,6-dioxopiperidin-3-yl)amino)-1-methyl-1H-pyrazol-5-yl)allyl)carbamate

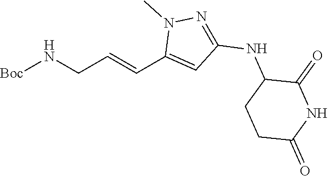 [SMILES: Cn1nc(NC2CCC(=O)NC2=O)cc1/C=C/CNC(=O)OC(C)(C)C]

To a mixture of tert-butyl (E)-(3-(4,4,5,5-tetramethyl-1,3,2-dioxaborolan-2-yl)allyl)carbamate (85 mg, 300 μmol, 1.0 eq.) and 3-((5-bromo-1-methyl-1H-pyrazol-3-yl)amino)piperidine-2,6-dione (86 mg, 300 μmol, 1.0 eq.) in 1,4-dioxane (5 mL) and water (0.5 mL) was added CsF (137 mg, 900 μmol, 3.0 eq.) and Pd(dppf)Cl₂ (33 mg, 45 μmol, 3.0 eq.) at room temperature. The mixture was stirred at 90° C. under N₂ for 2 hours. The resulting solution was diluted with 30 mL of water and extracted with EtOAc (30 mL×3). The combined organic layers were washed with brine (50 mL), dried over Na₂SO₄, and concentrated under reduced pressure. The residue was purified by flash column chromatography on silica gel (100-200 mesh silica gel, 0-75% EtOAc in PE) to give tert-butyl (E)-(3-(3-((2,6-dioxopiperidin-3-yl)amino)-1-methyl-1H-pyrazol-5-yl)allyl)carbamate (30 mg, 28% yield) as a yellow solid.

LC-MS (ESI): mass calcd. for C₁₇H₂₅N₅O₄, 363.2; m/z found, 364.1 [M+H]⁺.

### Step C: (E)-3-((5-(3-Aminoprop-1-en-1-yl)-1-methyl-1H-pyrazol-3-yl)amino)piperidine-2,6-dione

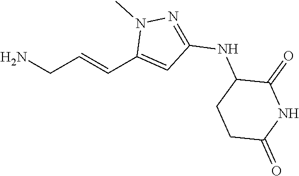 [SMILES: Cn1nc(NC2CCC(=O)NC2=O)cc1/C=C/CN]

To a mixture of tert-butyl (E)-(3-(3-((2,6-dioxopiperidin-3-yl)amino)-1-methyl-1H-pyrazol-5-yl)allyl)carbamate (30 mg, 82.5 μmol, 1.0 eq.) in DCM (3 mL) was added TFA (1 mL) at room temperature. The mixture was stirred at room temperature under N₂ for 1 hour. The mixture was concentrated under reduced pressure to give (E)-3-((5-(3-aminoprop-1-en-1-yl)-1-methyl-1H-pyrazol-3-yl)amino)piperidine-2,6-dione (20 mg, 92% yield crude) as a yellow solid.

LC-MS (ESI): mass calcd. for C₁₂H₁₇N₅O₂, 263.1; m/z found, 264.3 [M+H]⁺.

### Step D: (E)-N-(3-(3-((2,6-Dioxopiperidin-3-yl)amino)-]-methyl-1H-pyrazol-5-yl)allyl)-5-(8-(7-isopropyl-],3-dimethyl-2-oxo-2,3-dihydro-]H-benzo[d]imidazol-5-yl)isoquinolin-3-yl)picolinamide

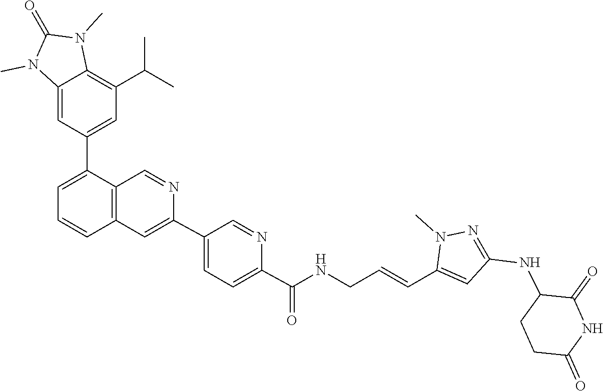 [SMILES: CC(C)c1cc(-c2cccc3cc(-c4ccc(C(=O)NC/C=C/c5cc(NC6CCC(=O)NC6=O)nn5C)nc4)ncc23)cc2c1n(C)c(=O)n2C]

To a mixture of 5-(8-(7-isopropyl-1,3-dimethyl-2-oxo-2,3-dihydro-1H-benzo[d]imidazol-5-yl)isoquinolin-3-yl)picolinic acid (15 mg, 33.1 μmol, 1.0 eq.; Intermediate 2) and (E)-3-((5-(3-aminoprop-1-en-1-yl)-1-methyl-1H-pyrazol-3-yl)amino)piperidine-2,6-dione (18 mg, 66.3 μmol, 2.0 eq.) in DMF (3 mL) was added DIPEA (17.3 μL, 99.4 μmol, 3.0 eq.) and HATU (15 mg, 39.8 μmol, 1.2 eq.) at 0° C. The mixture was stirred at room temperature under N₂ for 1 hour. The resulting solution was diluted with 30 mL of water and extracted with EtOAc (30 mL×3). The combined organic layers were washed with brine (50 mL), dried over Na₂SO₄, and concentrated under reduced pressure. The residue was purified by prep-HPLC (Triant C18 5 m column, 0-60% MeCN in H₂O with 0.1% FA) to give (E)-N-(3-(3-((2,6-dioxopiperidin-3-yl)amino)-1-methyl-1H-pyrazol-5-yl)allyl)-5-(8-(7-isopropyl-1,3-dimethyl-2-oxo-2,3-dihydro-1H-benzo[d]imidazol-5-yl)isoquinolin-3-yl)picolinamide (5.6 mg, 23% yield) as a white solid.

LC-MS (ESI): mass calcd. for C₃₉H₃₉N₉O₄, 697.3; m/z found, 698.2 [M+H]⁺.

¹H NMR (400 MHz, DMSO-d₆) δ 10.73 (s, 1H), 9.48 (s, 1H), 9.39 (s, 1H), 9.12 (t, J=5.9 Hz, 1H), 8.81-8.72 (m, 2H), 8.20 (d, J=8.1 Hz, 1H), 8.10 (d, J=8.2 Hz, 1H), 7.91 (t, J=7.7 Hz, 1H), 7.68 (d, J=7.1 Hz, 1H), 7.28 (s, 1H), 7.21 (s, 1H), 6.50 (d, J=15.8 Hz, 1H), 6.23 -6.10 (m, 1H), 5.73 (s, 1H), 5.43 (d, J=6.6 Hz, 1H), 4.22-4.07 (m, 3H), 3.79-3.72 (m, 1H), 3.66 (s, 3H), 3.58 (s, 3H), 3.39 (s, 3H), 2.71-2.64 (m, 1H), 2.57-2.53 (m, 1H), 2.23-2.16 (m, 1H), 1.92-1.82 (m, 1H), 1.35 (d, J=6.8 Hz, 6H).

### Example A17. N-(3-(1-(2,6-Dioxopiperidin-3-yl)-1H-benzo[d][1,2,3]triazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

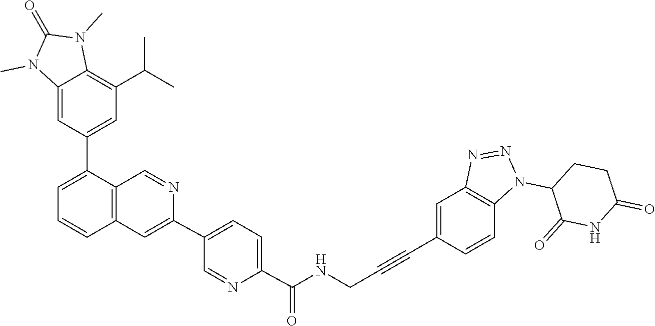 [SMILES: CC(C)c1cc(-c2cccc3cc(-c4ccc(C(=O)NCC#Cc5ccc6c(c5)nnn6C5CCC(=O)NC5=O)nc4)ncc23)cc2c1n(C)c(=O)n2C]

N-(3-(1-(2,6-Dioxopiperidin-3-yl)-1H-benzo[d][1,2,3]triazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 3-(5-bromo-1H-benzo[d][1,2,3]triazol-1-yl)piperidine-2,6-dione (Example A3, Step A) according to the procedures described in Example A1, Steps B-D. The title compound (2.3 mg, 8% yield over 3 steps) was obtained as a white solid.

LC-MS (ESI): mass calcd. for C₄₁H₃₅N₉O₄, 717.3; m/z found, 718.5 [M+H]⁺.

¹H NMR (400 MHz, DMSO-d₆) δ 11.30 (s, 1H), 9.50 (d, J=1.6 Hz, 1H), 9.41-9.37 (m, 2H), 8.84-8.79 (m, 1H), 8.77 (s, 1H), 8.28-8.14 (m, 2H), 8.10 (d, J=8.3 Hz, 1H), 7.92 (t, J=7.7 Hz, 1H), 7.84 (d, J=8.7 Hz, 1H), 7.69 (d, J=7.0 Hz, 1H), 7.62 (d, J=8.6 Hz, 1H), 7.28 (s, 1H), 7.21 (s, 1H), 6.30-6.12 (m, 1H), 4.43 (d, J=5.8 Hz, 2H), 3.78-3.73 (m, 1H), 3.66 (s, 3H), 3.39 (s, 3H), 2.99-2.87 (m, 2H), 2.81-2.72 (m, 1H), 2.46-2.40 (m, 1H), 1.35 (d, J=6.8 Hz, 6H).

### Example A18. N-(3-(3-(2,6-Dioxopiperidin-3-yl)-1-methyl-2-oxo-2,3-dihydro-1H-benzo[d]imidazol-5-yl)prop-2-yn-1-yl)-5-(8-(7-isopropyl-1,3-dimethyl-2-oxo-2,3-dihydro-1H-benzo[djimidazol-5-yl)isoquinolin-3-yl)picolinamide

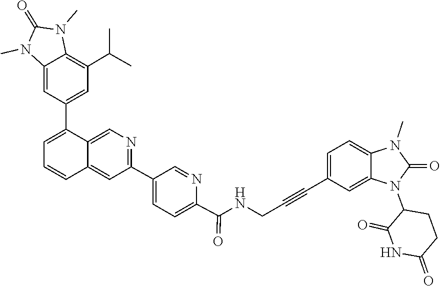 [SMILES: CC(C)c1cc(-c2cccc3cc(-c4ccc(C(=O)NCC#Cc5ccc6c(c5)n(C5CCC(=O)NC5=O)c(=O)n6C)nc4)ncc23)cc2c1n(C)c(=O)n2C]

N-(3-(3-(2,6-Dioxopiperidin-3-yl)-1-methyl-2-oxo-2,3-dihydro-1H-benzo[d]imidazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 5-bromo-1-methyl-1,3-dihydro-2H-benzo[d]imidazol-2-one according to the procedures described in Example A14, Steps A D. The title compound (2.3 mg, 3% yield over 4 steps) was obtained as a white solid.

LC-MS (ESI): mass calcd. for C₄₃H₃₈N₈O₅, 746.3; m/z found, 747.5 [M+H]⁺.

¹H NMR (400 MHz, DMSO-d₆) δ 11.07 (s, 1H), 9.50-9.47 (m, 1H), 9.40 (s, 1H), 9.31 (t, J=5.9 Hz, 1H), 8.82-8.75 (m, 2H), 8.22 (d, J=8.4 Hz, 1H), 8.12-8.08 (m, 1H), 7.94-7.89 (m, 1H), 7.70-7.66 (m, 1H), 7.29-7.24 (m, 2H), 7.22-7.17 (m, 3H), 5.42-5.35 (m, 1H), 4.37 (d, 2H), 3.79-3.72 (m, 1H), 3.66 (s, 3H), 3.39 (s, 3H), 3.34 (s, 3H), 2.88-2.77 (m, 1H), 2.75-2.66 (m, 1H), 2.64-2.57 (m, 1H), 2.03-1.97 (m, 1H), 1.34 (d, J=6.8 Hz, 6H).

### Example A19. N-(3-(3-Cyano-1-(2,6-dioxopiperidin-3-yl)-1H-indol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

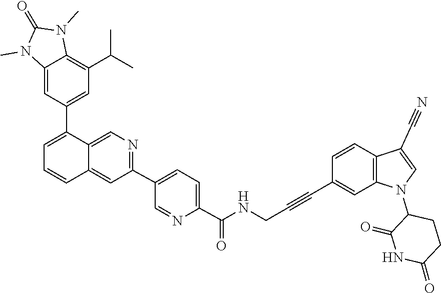 [SMILES: CC(C)c1cc(-c2cccc3cc(-c4ccc(C(=O)NCC#Cc5ccc6c(C#N)cn(C7CCC(=O)NC7=O)c6c5)nc4)ncc23)cc2c1n(C)c(=O)n2C]

N-(3-(3-Cyano-1-(2,6-dioxopiperidin-3-yl)-1H-indol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-1H-indole-3-carbonitrile according to the procedures described in Example A14, Steps A-D. The title compound (11.5 mg, 8% yield over 4 steps) was obtained as a yellow solid.

LC-MS (ESI): mass calcd. for C₄₄H₃₆N₈O₄, 740.3; m/z found, 741.1 [M+H]⁺.

¹H NMR (400 MHz, DMSO-d₆) δ 11.19 (s, 1H), 9.49 (s, 1H), 9.40 (s, 1H), 9.36 (t, J=5.9 Hz, 1H), 8.83-8.75 (m, 2H), 8.44 (s, 1H), 8.22 (d, J=8.2 Hz, 1H), 8.10 (d, J=8.4 Hz, 1H), 7.92 (t, J=7.6 Hz, 1H), 7.87 (s, 1H), 7.70-7.64 (m, 2H), 7.33 (d, J=8.3 Hz, 1H), 7.28 (s, 1H), 7.21 (s, 1H), 5.84 (dd, J=12.8, 4.8 Hz, 1H), 4.40 (d, J=5.8 Hz, 2H), 3.79-3.72 (m, 1H), 3.66 (s, 3H), 3.39 (s, 3H), 2.90-2.81 (m, 1H), 2.75-2.67 (m, 2H), 2.27-2.19 (m, 1H), 1.34 (d, J=6.7 Hz, 6H).

### Example A20. N-(3-(1-(2,6-Dioxopiperidin-3-yl)-1H-pyrazolo[4,3-b]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

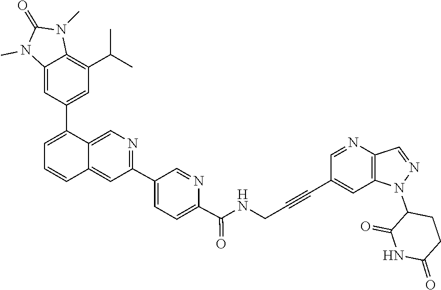 [SMILES: CC(C)c1cc(-c2cccc3cc(-c4ccc(C(=O)NCC#Cc5cnc6cnn(C7CCC(=O)NC7=O)c6c5)nc4)ncc23)cc2c1n(C)c(=O)n2C]

N-(3-(1-(2,6-Dioxopiperidin-3-yl)-1H-pyrazolo[4,3-b]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-1H-pyrazolo[4,3-b]pyridine according to the procedures described in Example A1, Steps A-D. The title compound (10 mg, 3% yield over 4 steps) was obtained as a white solid.

LC-MS (ESI): mass calcd. for C₄₁H₃₅N₉O₄, 717.3; m/z found, 718 [M+H]⁺.

¹H NMR (400 MHz, DMSO-d₆) δ 11.14 (s, 1H), 9.50 (d, J=1.7 Hz, 1H), 9.43 (t, J=5.9 Hz, 1H), 9.40 (s, 1H), 8.82-8.78 (m, 1H), 8.76 (s, 1H), 8.57 (d, J=1.6 Hz, 1H), 8.40 (s, 1H), 8.33 (s, 1H), 8.23 (d, J=8.3 Hz, 1H), 8.10 (d, J=8.3 Hz, 1H), 7.94-7.89 (m, 1H), 7.68 (d, J=7.1 Hz, 1H), 7.28 (d, J=1.3 Hz, 1H), 7.21 (d, J=1.2 Hz, 1H), 5.97-5.84 (m, 1H), 4.45 (d, J=5.8 Hz, 2H), 3.78-3.73 (m, 1H), 3.66 (s, 3H), 3.39 (s, 3H), 2.84-2.73 (m, 3H), 2.34-2.26 (m, 1H), 1.34 (d, J=6.8 Hz, 6H).

### Example A21. N-(3-(4-Cyano-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

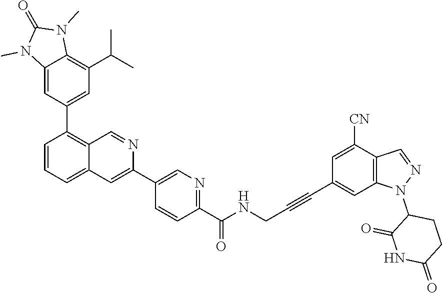 [SMILES: CC(C)c1cc(-c2cccc3cc(-c4ccc(C(=O)NCC#Cc5cc(C#N)c6cnn(C7CCC(=O)NC7=O)c6c5)nc4)ncc23)cc2c1n(C)c(=O)n2C]

N-(3-(4-Cyano-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-1H-indazole-4-carbonitrile according to the procedures described in Example A1, Steps A-D. The title compound (8.4 mg, 11% yield over 4 steps) was obtained as a white solid.

LC-MS (ESI): mass calcd. for C₄₃H₃₅N₉O₄, 741.3; m/z found, 742.3 [M+H]⁺.

¹H NMR (400 MHz, DMSO-d₆) δ 11.15 (s, 1H), 9.50 (s, 1H), 9.44-9.37 (m, 2H), 8.83-8.78 (m, 1H), 8.78-8.75 (m, 1H), 8.40 (s, 1H), 8.27-8.20 (m, 2H), 8.13-8.07 (m, 1H), 7.92 (t, 1H), 7.83 (s, 1H), 7.68 (d, J=7.2 Hz, 1H), 7.28 (s, 1H), 7.21 (s, 1H), 6.06-5.97 (m, 1H), 4.43 (d, J=5.9 Hz, 2H), 3.79-3.71 (m, 1H), 3.66 (s, 3H), 3.38 (s, 3H), 2.83-2.69 (m, 3H), 2.35-2.26 (m, 1H), 1.34 (d, J=6.8 Hz, 6H).

### Example A22. N-(3-(1-(2,6-Dioxopiperidin-3-yl)-1H-pyrrolo[3,2-c]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

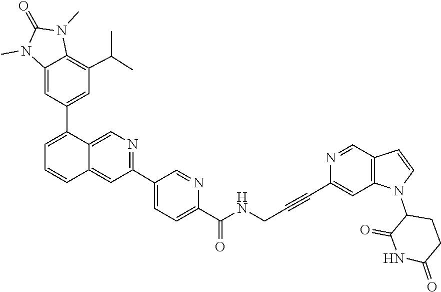 [SMILES: CC(C)c1cc(-c2cccc3cc(-c4ccc(C(=O)NCC#Cc5cc6c(ccn6C6CCC(=O)NC6=O)cn5)nc4)ncc23)cc2c1n(C)c(=O)n2C]

N-(3-(1-(2,6-Dioxopiperidin-3-yl)-1H-pyrrolo[3,2-c]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-1H-pyrrolo[3,2-c]pyridine according to the procedures described in Example A1, Steps A D. The title compound (9.1 mg, 12% yield over 4 steps) was obtained as a yellow solid.

LC-MS (ESI): mass calcd. for C₄₂H₃₆N₈O₄, 716.3; m/z found, 717.6 [M+H]⁺.

¹H NMR (400 MHz, DMSO-d₆) δ 11.21 (s, 1H), 9.52-9.46 (m, 2H), 9.40 (s, 1H), 9.07 (s, 1H), 8.82 (d, J=8.4 Hz, 1H), 8.78 (s, 1H), 8.24 (d, J=8.5 Hz, 1H), 8.17-8.08 (m, 2H), 7.96-7.88 (m, 2H), 7.70 (d, J=7.3 Hz, 1H), 7.29 (s, 1H), 7.21 (s, 1H), 6.98 (s, 1H), 5.90-5.82 (m, 1H), 4.50-4.44 (m, 2H), 3.79-3.74 (m, 1H), 3.66 (s, 3H), 3.39 (s, 3H), 2.84-2.77 (m, 2H), 2.75-2.71 (m, 1H), 2.24-2.19 (m, 1H), 1.35 (d, J=6.0 Hz, 6H).

### Example A23. N-(3-(3-(2,6-Dioxopiperidin-3-yl)benzofuran-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: Ethyl 2-(5-bromobenzofuran-3-yl)acetate

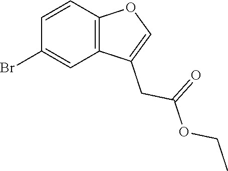 [SMILES: CCOC(=O)Cc1coc2ccc(Br)cc12]

A solution of 5-bromobenzofuran-3(2H)-one (4 g, 18.8 mmol, 1.0 eq.) and ethyl (triphenylphosphoranylidene)acetate (13 g, 37.6 mmol, 2.0 eq.) in toluene (50 mL) was stirred at 110° C. for 48 hours. The reaction mixture was concentrated in vacuo and purified by flash column chromatography on silica gel (100-200 mesh silica gel, 0-50% EtOAc in PE) to give ethyl 2-(5-bromobenzofuran-3-yl)acetate (4 g, 75% yield) as a white solid.

LC-MS (ESI): mass calcd. for C₁₂H₁₁BrO₃, 282.0/284.0; m/z found, No signal.

¹H NMR (400 MHz, DMSO-d₆) δ 7.89 (s, 1H), 7.76 (d, J=2.0 Hz, 1H), 7.50 (d, J=8.8 Hz, 1H), 7.40 (dd, J=8.8, 2.0 Hz, 1H), 4.05 (q, J=7.2 Hz, 2H), 3.73 (s, 2H), 1.14 (t, J=7.2 Hz, 3H).

### Step B: 3-(5-Bromobenzofuran-3-yl)piperidine-2,6-dione

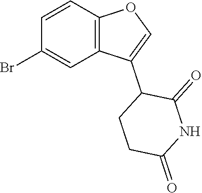 [SMILES: O=C1CCC(c2coc3ccc(Br)cc23)C(=O)N1]

To a mixture of ethyl 2-(5-bromobenzofuran-3-yl)acetate (3.5 g, 12.4 mmol, 1.0 eq.) in DMF (40 mL) was added t-BuOK (1.4 g, 12.4 mmol, 1.0 eq.) and acrylamide (879 mg, 12.4 mmol, 1.0 eq.) at 0° C. The reaction mixture was then stirred at 25° C. for 2 hours. The resulting solution was diluted with 100 mL of H₂O and extracted with EtOAc (100 mL×3). The combined organic layers were washed with brine, dried over Na₂SO₄, and concentrated under reduced pressure. The residue was purified by flash column chromatography on silica gel (100-200 mesh silica gel, 0-70% EtOAc in PE) to give 3-(5-bromobenzofuran-3-yl)piperidine-2,6-dione (1 g, 26% yield) as a white solid.

LC-MS (ESI): mass calcd. for C₁₃H₁₀BrNO₃, 307.0/309.0; m/z found, m/z 308.2/310.2 [M+H]⁺.

¹H NMR (400 MHz, DMSO-d₆) δ 10.90 (s, 1H), 7.97 (s, 1H), 7.86 (d, J=2.0 Hz, 1H), 7.57 (d, J=8.8 Hz, 1H), 7.46 (dd, J=8.8, 2.0 Hz, 1H), 4.15 (dd, J=12.4, 4.8 Hz, 1H), 2.77-2.67 (m, 1H), 2.57 (dd, J=13.6, 3.6 Hz, 1H), 2.39-2.31 (m, 1H), 2.14-2.04 (m, 1H).

### Steps C-E: N-(3-(3-(2,6-Dioxopiperidin-3-yl)benzofuran-5-yl)prop-2-yn-]-yl)-5-(8-(7-isopropyl-1,3-dimethyl-2-oxo-2,3-dihydro-]H-benzo[d]imidazol-5-yl)isoquinolin-3-yl)picolinamide

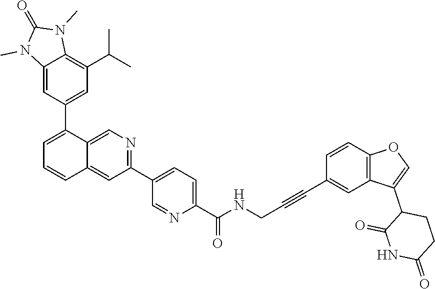 [SMILES: CC(C)c1cc(-c2cccc3cc(-c4ccc(C(=O)NCC#Cc5ccc6occ(C7CCC(=O)NC7=O)c6c5)nc4)ncc23)cc2c1n(C)c(=O)n2C]

N-(3-(3-(2,6-Dioxopiperidin-3-yl)benzofuran-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 3-(5-bromobenzofuran-3-yl)piperidine-2,6-dione according to the procedures described in Example A1, Steps B D. The title compound (23 mg, 18% yield over 3 steps) was obtained as a white solid.

LC-MS (ESI): mass calcd. for C₄₃H₃₆N₆O₅, 716.3; m/z found, 717.4 [M+H]⁺.

¹H NMR (400 MHz, DMSO-d₆) δ 10.88 (s, 1H), 9.49 (s, 1H), 9.40 (s, 1H), 9.34 (t, J=5.9 Hz, 1H), 8.84-8.78 (m, 1H), 8.77 (s, 1H), 8.22 (d, J=8.2 Hz, 1H), 8.10 (d, J=8.1 Hz, 1H), 7.97-7.89 (m, 2H), 7.73-7.67 (m, 2H), 7.58 (d, J=8.6 Hz, 1H), 7.37 (d, J=8.6 Hz, 1H), 7.28 (s, 1H), 7.21 (s, 1H), 4.38 (d, J=5.9 Hz, 2H), 4.15 (dd, J=12.2, 5.0 Hz, 1H), 3.79-3.72 (m, 1H), 3.66 (s, 3H), 3.39 (s, 3H), 2.74-2.67 (m, 1H), 2.60-2.55 (m, 1H), 2.36-2.30 (m, 1H), 2.12-2.04 (m, 1H), 1.35 (d, J=6.8 Hz, 6H).

### Example A24. N-(3-(3-((2,6-Dioxopiperidin-3-yl)amino)-1-methyl-1H-pyrazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

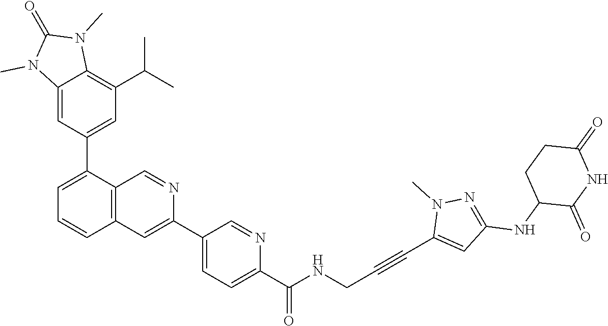 [SMILES: CC(C)c1cc(-c2cccc3cc(-c4ccc(C(=O)NCC#Cc5cc(NC6CCC(=O)NC6=O)nn5C)nc4)ncc23)cc2c1n(C)c(=O)n2C]

N-(3-(3-((2,6-Dioxopiperidin-3-yl)amino)-1-methyl-1H-pyrazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 3-((5-bromo-1-methyl-1H-pyrazol-3-yl)amino)piperidine-2,6-dione (Example A16, Step A) according to the procedures described in Example A1, Steps B-D. The title compound (22.5 mg, 26% yield over 3 steps) was obtained as a white solid.

LC-MS (ESI): mass calcd. for C₃₉H₃₇N₉O₄, 695.3; m/z found, 696.3 [M+H]⁺.

¹H NMR (400 MHz, DMSO-d₆) δ 10.78-10.69 (m, 1H), 9.49 (s, 1H), 9.42-9.38 (m, 2H), 8.82-8.76 (m, 2H), 8.22 (d, J=8.2 Hz, 1H), 8.10 (d, J=8.4 Hz, 1H), 7.92 (t, 1H), 7.68 (d, J=7.1 Hz, 1H), 7.28 (s, 1H), 7.21 (s, 1H), 5.76 (s, 1H), 5.65 (d, J=6.9 Hz, 1H), 4.40 (d, J=5.9 Hz, 2H), 4.21-4.14 (m, 1H), 3.79-3.72 (m, 1H), 3.66 (s, 3H), 3.64 (s, 3H), 3.39 (s, 3H), 2.69-2.64 (m, 1H), 2.56-2.53 (m, 1H), 2.25-2.13 (m, 1H), 1.94-1.78 (m, 1H), 1.35 (d, J=6.8 Hz, 6H).

### Example A25. N-(3-(3-(2,6-Dioxopiperidin-3-yl)imidazo[1,2-a]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: Ethyl 2-(6-bromoimidazo[ ],2-a]pyridin-3-yl)acetate

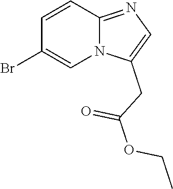 [SMILES: CCOC(=O)Cc1cnc2ccc(Br)cn12]

To a solution of 5-bromopyridin-2-amine (5 g, 28.9 mmol, 1.0 eq.) in MeCN (50 mL) was added ethyl (E)-4-oxobut-2-enoate (3.7 g, 28.9 mmol, 1.0 eq.). The mixture was stirred at 80° C. for 16 hours under N₂. The mixture was then concentrated under reduced pressure and purified by flash chromatography (100-200 mesh silica gel, 0-50% EtOAc in PE) to afford ethyl 2-(6-bromoimidazo[1,2-a]pyridin-3-yl)acetate (2.7 g, 33% yield) as a yellow solid.

LC-MS (ESI): mass calcd. for C₁₁H₁₁BrN₂O₂, 282.0/284.0; m/z found, 283.0/285.0 [M+H]⁺.

### Step B: 3-(6-Bromoimidazo[1,2-a]pyridin-3-yl)piperidine-2,6-dione

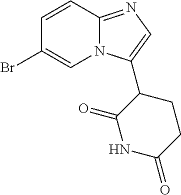 [SMILES: O=C1CCC(c2cnc3ccc(Br)cn23)C(=O)N1]

To a solution of ethyl 2-(6-bromoimidazo[1,2-a]pyridin-3-yl)acetate (300 mg, 1.1 mmol, 1.0 eq.) in THF (10 mL) was added LDA (1.1 mL, 2M in THF, 2.2 mmol, 2.0 eq.) at 25° C.

The mixture was stirred at this temperature for 30 mins under N₂. At this point, acrylamide (151 mg, 2.2 mmol, 2.0 eq.) was added to the solution. The reaction mixture was stirred at 50° C. for 2 hours under N₂. The resulting solution was diluted with 20 mL of water and extracted with EtOAc (10 mL×3). The combined organic layers were washed with brine (20 mL), dried over Na₂SO₄, and concentrated under reduced pressure. The residue was purified by flash column chromatography on silica gel (100-200 mesh silica gel, 0-100% EtOAc in PE) to give 3-(6-bromoimidazo[1,2-a]pyridin-3-yl)piperidine-2,6-dione (40 mg, 12% yield) as a yellow solid.

LC-MS (ESI): mass calcd. for C₁₂H₁₀BrN₃O₂, 307.0/309.0; m/z found, 308.0/310.0 [M+H]⁺.

### Steps C E: N-(3-(3-(2,6-Dioxopiperidin-3-yl)imidazo[1,2-a]pyridin-6-yl)prop-2-yn-1-yl)-5-(8-(7-isopropyl-],3-dimethyl-2-oxo-2,3-dihydro-1H-benzo[d]imidazol-5-yl)isoquinolin-3-yl)picolinamide

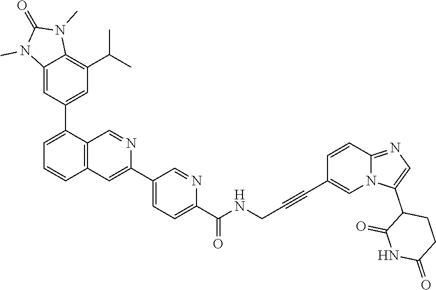 [SMILES: CC(C)c1cc(-c2cccc3cc(-c4ccc(C(=O)NCC#Cc5ccc6ncc(C7CCC(=O)NC7=O)n6c5)nc4)ncc23)cc2c1n(C)c(=O)n2C]

N-(3-(3-(2,6-Dioxopiperidin-3-yl)imidazo[1,2-a]pyridin-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 3-(6-bromoimidazo[1,2-a]pyridin-3-yl)piperidine-2,6-dione according to the procedures described in Example A1, Steps B-D. The title compound (25.4 mg, 13% yield over 3 steps) was obtained as a white solid.

LC-MS (ESI): mass calcd. for C₄₂H₃₆N₈O₄, 716.3; m/z found, 717.2 [M+H]⁺.

¹H NMR (400 MHz, DMSO-d₆) δ 11.01 (s, 1H), 9.50 (d, J=1.6 Hz, 1H), 9.44-9.37 (m, 2H), 8.89-8.75 (m, 3H), 8.22 (d, J=8.0 Hz, 1H), 8.10 (d, J=8.2 Hz, 1H), 7.95-7.88 (m, 1H), 7.81 (s, 1H), 7.75 (d, J=9.7 Hz, 1H), 7.69 (d, J=6.4 Hz, 1H), 7.61-7.50 (m, 1H), 7.28 (s, 1H), 7.21 (s, 1H), 4.64 (d, J=8.6 Hz, 1H), 4.42 (d, J=5.9 Hz, 2H), 3.79-3.72 (m, 1H), 3.66 (s, 3H), 3.39 (s, 3H), 2.71-2.66 (m, 2H), 2.56-2.52 (m, 1H), 2.21-2.15 (m, 1H), 1.35 (d, J=6.8 Hz, 6H).

### Example A26. N-(3-(1-(2,6-Dioxopiperidin-3-yl)-4-methoxy-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

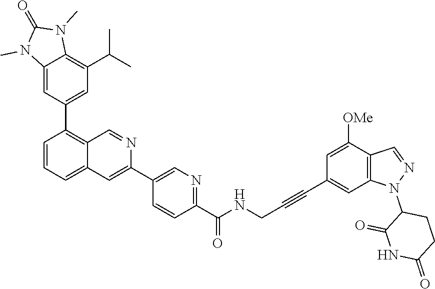 [SMILES: COc1cc(C#CCNC(=O)c2ccc(-c3cc4cccc(-c5cc(C(C)C)c6c(c5)n(C)c(=O)n6C)c4cn3)cn2)cc2c1cnn2C1CCC(=O)NC1=O]

N-(3-(1-(2,6-Dioxopiperidin-3-yl)-4-methoxy-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-4-methoxy-1H-indazole according to the procedures described in Example A1, Steps A-D. The title compound (39.4 mg, 28% yield over 4 steps) was obtained as a white solid.

LC-MS (ESI): mass calcd. for C₄₃H₃₈N₈O₅, 746.3; m/z found, 747.5 [M+H]⁺.

¹H NMR (400 MHz, DMSO-d₆) δ 11.07 (s, 1H), 9.50 (d, J=1.6 Hz, 1H), 9.42-9.35 (m, 2H), 8.81 (dd, J=8.2, 2.1 Hz, 1H), 8.77 (s, 1H), 8.24 (d, J=8.3 Hz, 1H), 8.15-8.07 (m, 2H), 7.92 (t, 1H), 7.69 (d, J=7.2 Hz, 1H), 7.40 (s, 1H), 7.29 (s, 1H), 7.21 (s, 1H), 6.61 (s, 1H), 5.87-5.80 (m, 1H), 4.41 (d, J=5.8 Hz, 2H), 3.95 (s, 3H), 3.80-3.72 (m, 1H), 3.67 (s, 3H), 3.39 (s, 3H), 2.82-2.76 (m, 1H), 2.74-2.67 (m, 2H), 2.28-2.21 (m, 1H), 1.35 (d, J=6.8 Hz, 6H).

### Example A27. N-(3-(1-(2,6-Dioxopiperidin-3-yl)-5-methoxy-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

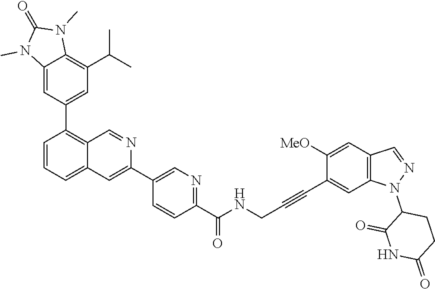 [SMILES: COc1cc2cnn(C3CCC(=O)NC3=O)c2cc1C#CCNC(=O)c1ccc(-c2cc3cccc(-c4cc(C(C)C)c5c(c4)n(C)c(=O)n5C)c3cn2)cn1]

N-(3-(1-(2,6-Dioxopiperidin-3-yl)-5-methoxy-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-5-methoxy-1H-indazole according to the procedures described in Example A1, Steps A-D. The title compound (10.5 mg, 14% yield over 4 steps) was obtained as a white solid.

LC-MS (ESI): mass calcd. for C₄₃H₃₈N₈O₅, 746.3; m/z found, 747.5 [M+H]⁺.

¹H NMR (400 MHz, DMSO-d₆) δ 11.05 (s, 1H), 9.50 (d, J=1.7 Hz, 1H), 9.40 (s, 1H), 9.35 (t, J=6.0 Hz, 1H), 8.80 (dd, J=8.2, 2.2 Hz, 1H), 8.77 (s, 1H), 8.23 (d, J=8.2 Hz, 1H), 8.13-8.08 (m, 1H), 8.02 (s, 1H), 7.95-7.89 (m, 1H), 7.77 (s, 1H), 7.69 (d, J=7.1 Hz, 1H), 7.30-7.25 (m, 2H), 7.21 (d, J=1.4 Hz, 1H), 5.89-5.79 (m, 1H), 4.42 (d, J=5.9 Hz, 2H), 3.84 (s, 3H), 3.78-3.72 (m, 1H), 3.66 (s, 3H), 3.39 (s, 3H), 2.80-2.66 (m, 3H), 2.27-2.18 (m, 1H), 1.35 (d, J=6.8 Hz, 6H).

### Example A28. N-(3-(3-((Dimethylamino)methyl)-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: 1-(6-Bromo-]H-indazol-3-yl)-N,N-dimethylmethanamine

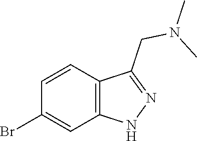

To a solution of 6-bromo-1H-indazole-3-carbaldehyde (2 g, 8.9 mmol, 1 eq.) in THF (8 mL) was added acetic acid (0.8 mL, 13.3 mmol, 1.5 eq.), dimethylamine (8.9 mL, 17.8 mmol, 2 eq., 2.0 M in THF), and NaBH(OAc)₃ (1.9 g, 8.9 mmol, 1 eq.). The reaction mixture was stirred at 25° C. for 12 hours under N₂ atmosphere. The reaction mixture was then concentrated and purified by flash column chromatography on silica gel (100-200 mesh silica gel, 0-6% DCM in MeOH) to give 1-(6-bromo-1H-indazol-3-yl)-N,N-dimethylmethanamine (1.5 g, 66% yield) as a brown solid.

LC-MS (ESI): mass calcd. for C₁₀H₁₂BrN₃, 253.0/255.0; m/z found, 254.0/256.0 [M+H]⁺.

¹H NMR (400 MHz, DMSO-d₆) δ 12.93 (s, 1H), 7.78 (d, J=8.5 Hz, 1H), 7.70 (d, J=1.1 Hz, 1H), 7.21 (dd, J=8.6, 1.6 Hz, 1H), 3.73 (s, 2H), 2.17 (s, 6H).

### Steps B-E: N-(3-(3-((Dimethylamino)methyl)-1-(2,6-dioxopiperidin-3-yl)-]H-indazol-6-yl)prop-2-yn-1-yl)-5-(8-(7-isopropyl-],3-dimethyl-2-oxo-2,3-dihydro-]H-benzo[d]imidazol-5-yl)isoquinolin-3-yl)picolinamide

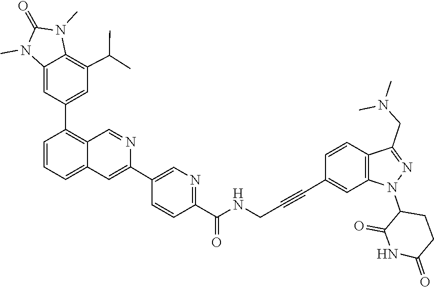 [SMILES: CC(C)c1cc(-c2cccc3cc(-c4ccc(C(=O)NCC#Cc5ccc6c(CN(C)C)nn(C7CCC(=O)NC7=O)c6c5)nc4)ncc23)cc2c1n(C)c(=O)n2C]

N-(3-(3-((Dimethylamino)methyl)-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 1-(6-bromo-1H-indazol-3-yl)-N,N-dimethylmethanamine according to the procedures described in Example A1, Steps A-D. The title compound (23.6 mg, 13% yield over 4 steps) was obtained as a yellow solid.

LC-MS (ESI): mass calcd. for C₄₅H₄₃N₉O₄, 773.3; m/z found, 774.7 [M+H]⁺.

¹H NMR (400 MHz, DMSO-d₆) δ 11.15 (s, 1H), 9.93 (s, 1H), 9.50 (d, J=1.7 Hz, 1H), 9.42-9.38 (m, 2H), 8.81 (dd, J=8.2, 2.2 Hz, 1H), 8.77 (s, 1H), 8.23 (d, J=8.3 Hz, 1H), 8.10 (d, J=8.3 Hz, 1H), 7.99 (d, J=8.5 Hz, 1H), 7.95-7.90 (m, 2H), 7.70-7.68 (m, 1H), 7.35-7.30 (m, 1H), 7.28 (d, J=1.5 Hz, 1H), 7.21 (d, J=1.5 Hz, 1H), 6.05-5.92 (m, 1H), 4.72-4.51 (m, 2H), 4.42 (d, J=5.9 Hz, 2H), 3.79-3.72 (m, 1H), 3.66 (s, 3H), 3.39 (s, 3H), 2.84-2.69 (m, 9H), 2.35-2.28 (m, 1H), 1.35 (d, J=6.8 Hz, 6H).

### Example A29. N-(3-(3-((2,6-Dioxopiperidin-3-yl)oxy)-1-methyl-1H-pyrazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: 3-((tert-Butyldimethylsilyl)oxy)-1-methyl-1H-pyrazole

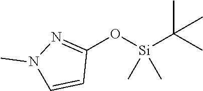

To a solution of 1-methyl-1H-pyrazol-3-ol (5.0 g, 51.0 mmol, 1.0 eq.) and TEA (10.3 g, 102.0 mmol, 2.0 eq.) in DCM (60 mL) was added TBSCI (8.4 g, 56.1 mmol, 1.1 eq.) at 0° C. The mixture was stirred at 25° C. for 4 hours under N₂ atmosphere. The resulting solution was diluted with 100 mL of water and extracted with EtOAc (100 mL×3). The combined organic layers were dried over Na₂SO₄ and concentrated under reduced pressure. The residue was purified by flash column chromatography on silica gel (100-200 mesh silica gel, 0-10% EtOAc in PE) to give 3-((tert-butyldimethylsilyl)oxy)-1-methyl-1H-pyrazole (10 g, 92% yield) as a yellow oil.

LC-MS (ESI): mass calcd. for C₁₀H₂MN₂₀Si, 212.1; m/z found, m/z 213 [M+H]⁺.

### Step B: 3-((tert-Butyldimethylsilyl)oxy)-5-iodo-]-methyl-1H-pyrazole

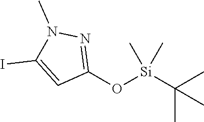

To a solution of 3-((tert-butyldimethylsilyl)oxy)-1-methyl-1H-pyrazole (5 g, 23.5 mmol, 1.0 eq.) in THF (60 mL) was added LDA (14.1 ml, 1.2 eq, 2 M in THF) at −78° C. The mixture was stirred at −78° C. for 1 h under N₂ atmosphere. Then, 12 (7.2 g, 28.3 mmol, 1.2 eq.) was added to this solution. The mixture was stirred at −78° C. for another 2 hours under N₂ atmosphere. The resulting solution was diluted with 50 mL of sat. NH₄Cl and extracted with EtOAc (100 mL×3). The combined organic layers were washed with brine, dried over Na₂SO₄, and concentrated under reduced pressure. The residue was purified by flash column chromatography on silica gel (100-200 mesh silica gel, 0-10% EtOAc in PE) to give 3-((tert-butyldimethylsilyl)oxy)-5-iodo-1-methyl-1H-pyrazole (4.5 g, 56% yield) as a yellow solid.

LC-MS (ESI): mass calcd. for C₁₀H₁₉IN₂OS, 338.0; m/z found, m/z 339 [M+H]⁺.

### Step C: 5-Iodo-]-methyl-]H-pyrazol-3-ol

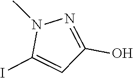

To a solution of 3-((tert-butyldimethylsilyl)oxy)-5-iodo-1-methyl-1H-pyrazole (4.5 g, 13.3 mmol, 1.0 eq.) in THF (50 mL) was added TBAF (4.2 g, 16.0 mmol, 1.2 eq.) at 25° C. The mixture was stirred at 25° C. for 2 hours under N₂ atmosphere. The resulting solution was diluted with 50 mL of H₂O and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-50% EtOAc in PE) to give 5-iodo-1-methyl-1H-pyrazol-3-ol (2.5 g, 84% yield) as a white solid.

LC-MS (ESI): mass calcd. for C₄H₅₁N₂O, 223.9; m/z found, m/z 225 [M+H]⁺.

### Step D: 3-((5-Iodo-1-methyl-1H-pyrazol-3-yl)oxy)piperidine-2,6-dione

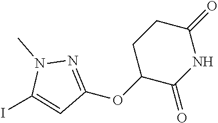 [SMILES: Cn1nc(OC2CCC(=O)NC2=O)cc1I]

To a mixture of 5-iodo-1-methyl-1H-pyrazol-3-ol (300 mg, 1.3 mmol, 1.0 eq.) and Cs₂CO₃ (436 mg, 1.3 mmol, 1.0 eq.) in DMF (5 mL) was added 3-bromopiperidine-2,6-dione (257 mg, 1.3 mmol, 1.0 eq.) at 25° C. The mixture was then stirred at 50° C. for 6 hours under N₂ atmosphere. The resulting solution was diluted with 50 mL of H₂O and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-50% EtOAc in PE) to afford 3-((5-iodo-1-methyl-1H-pyrazol-3-yl)oxy)piperidine-2,6-dione (160 mg, 36% yield) as a white solid.

LC-MS (ESI): mass calcd. for C₉H₁₀IN₃O₃, 335.0; m/z found, m/z 336 [M+H]⁺.

### Steps E G: N-(3-(3-((2,6-Dioxopiperidin-3-yl)oxy)-]-methyl-1H-pyrazol-5-yl)prop-2-yn-]-yl)-5-(8-(7-isopropyl-],3-dimethyl-2-oxo-2,3-dihydro-]H-benzo[d]imidazol-5-yl)isoquinolin-3-yl)picolinamide

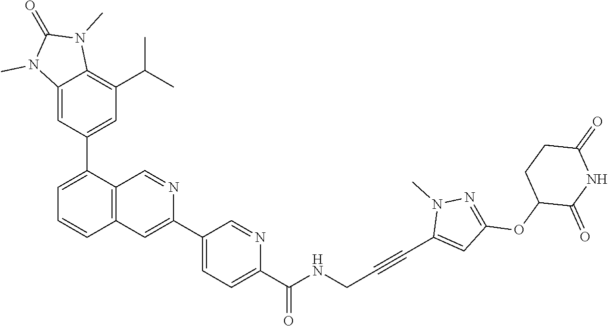 [SMILES: CC(C)c1cc(-c2cccc3cc(-c4ccc(C(=O)NCC#Cc5cc(OC6CCC(=O)NC6=O)nn5C)nc4)ncc23)cc2c1n(C)c(=O)n2C]

N-(3-(3-((2,6-Dioxopiperidin-3-yl)oxy)-1-methyl-1H-pyrazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 3-((5-iodo-1-methyl-1H-pyrazol-3-yl)oxy)piperidine-2,6-dione according to the procedures described in Example A1, Steps B D. The title compound(20 mg, 25% yield over 3 steps) was obtained as a white solid.

LC-MS (ESI): mass calcd. for C₃₉H₃₆N₈O₅, 696.3; m/z found, 697.3 [M+H]⁺.

¹H NMR (400 MHz, DMSO-d₆) δ 10.89 (s, 1H), 9.49 (d, J=1.8 Hz, 1H), 9.46-9.40 (m, 1H), 9.40 (s, 1H), 8.84-8.72 (m, 2H), 8.22 (d, J=8.2 Hz, 1H), 8.14-8.06 (m, 1H), 7.96-7.88 (m, 1H), 7.75-7.63 (m, 1H), 7.34-7.25 (m, 1H), 7.23-7.14 (m, 1H), 6.01 (s, 1H), 5.29-5.19 (m, 1H), 4.42 (d, J=5.8 Hz, 2H), 3.80-3.72 (m, 1H), 3.71 (s, 3H), 3.66 (s, 3H), 3.39 (s, 3H), 2.77-2.67 (m, 1H), 2.59-2.53 (m, 1H), 2.26-2.17 (m, 1H), 2.15-2.04 (m, 1H), 1.35 (d, J=6.8 Hz, 6H).

### Example A30. N-(3-(3-(2,6-Dioxopiperidin-3-yl)-1-methyl-1H-indazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)-3-methoxypicolinamide

### Step A: Methyl 5-bromo-3-methoxypicolinate

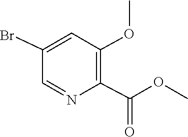

To a solution of 5-bromo-3-methoxypicolinic acid (1 g, 4.3 mmol, 1 eq.) in MeOH (10 mL) was added conc. H₂SO₄ (0.2 mL). The mixture was stirred at 70° C. for 2 hours. The resulting solution was diluted with sat. aq. NaHCO₃ (40 mL) and extracted with EtOAc (30 mL×3). The combined organic layers were washed with brine, dried over Na₂SO₄, and concentrated under reduced pressure. The residue was purified by flash column chromatography on silica gel (100-200 mesh silica gel, 0-20% EtOAc in PE) to afford methyl 5-bromo-3-methoxypicolinate (970 mg, 87% yield) as a yellow oil.

LC-MS (ESI): mass calcd. for C₈H₈BrNO₃, 245.0/247.0; m/z found, 246.1/248.1 [M+H]⁺.

### Step B: (5-Methoxy-6-(methoxycarbonyl)pyridin-3-yl)boronic acid

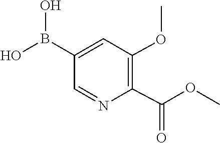

To a solution of methyl 5-bromo-3-methoxypicolinate (970 mg, 3.9 mmol, 1 eq.) in 1,4-dioxane (10 mL) was added bis(pinacolato)diborane (1.1 g, 4.3 mmol, 1.1 eq.), AcOK (1.2 g, 11.8 mmol, 3 eq.), and Pd(dppf)Cl₂ (322 mg, 394 μmol, 0.1 eq.) under N₂. The mixture was stirred at 100° C. for 2 hours. The mixture was filtered and concentrated under reduced pressure. The residue was purified by Prep-HPLC (Ci₈, 5-95% MeOH in H₂O with 0.1% HCOOH) to afford (5-methoxy-6-(methoxycarbonyl)pyridin-3-yl)boronic acid (770 mg, 88% yield) as a yellow oil.

LC-MS (ESI): mass calcd. for C₈H₁₀BNO₅, 211.1; m/z found, 212.0 [M+H]f.

### Step C: Methyl 5-(8-bromoisoquinolin-3-yl)-3-methoxypicolinate

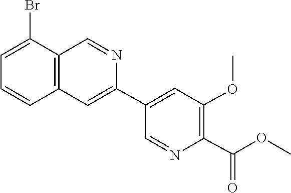 [SMILES: COC(=O)c1ncc(-c2cc3cccc(Br)c3cn2)cc1OC]

To a solution of 8-bromoisoquinolin-3-yl trifluoromethanesulfonate (928 mg, 2.6 mmol, 1 eq.; Intermediate 1, Step C) in DMF (22 mL) and water (2.2 mL) was added (5-methoxy-6-(methoxycarbonyl)pyridin-3-yl)boronic acid (550 mg, 2.6 mmol, 1 eq.), Pd(dppf)Cl₂ (191 mg, 261 μmol, 0.1 eq.), and K₃PO₄ (1.7 g, 7.8 mmol, 3 eq.) under N₂. The solution was stirred at 70° C. for 2 hours. The mixture was diluted with water and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-50% EtOAc in PE) to give methyl 5-(8-bromoisoquinolin-3-yl)-3-methoxypicolinate (574 mg, 56% yield) as a yellow solid.

LC-MS (ESI): mass calcd. for C₁₇H₁₃BrN₂O₃, 372.0/374.0; m/z found, 373.2/375.2 [M+H]⁺.

### Step D: Methyl 3-methoxy-5-(8-(4,4,5,5-tetramethyl-1,3,2-dioxaborolan-2-yl)isoquinolin-3-yl)picoiinate

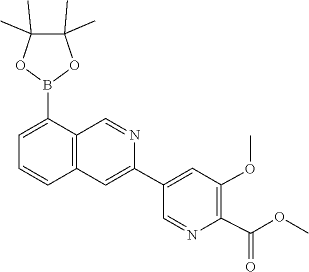

To a solution of methyl 5-(8-bromoisoquinolin-3-yl)-3-methoxypicolinate (420 mg, 1.1 mmol, 1 eq.) in 1,4-dioxane (10 mL) was added bis(pinacolato)diborane (857 mg, 3.4 mmol, 3 eq.), AcOK (331 mg, 3.3 mmol, 3 eq.), and Pd(dppf)Cl₂ (82 mg, 113 μmol, 0.1 eq.) under N₂. The mixture was stirred at 100° C. for 2 hours. The solution was filtered, and the filtrate was concentrated under reduced pressure. The residue was purified by flash chromatography (100-200 mesh silica gel, 0-50% EtOAc in PE) to afford methyl 3-methoxy-5-(8-(4,4,5,5-tetramethyl-1,3,2-dioxaborolan-2-yl)isoquinolin-3-yl)picolinate (242 mg, 49% yield) as a yellow solid.

LC-MS (ESI): mass calcd. for C₂₃H₂₅BN₂O₅, 420.2; m/z found, 421.3 [M+H]⁺.

### Step E: Methyl 5-(8-(7-isopropyl-1,3-dimethyl-2-oxo-2,3-dihydro-]H-benzo[d]imidazol-5-yl)isoquinolin-3-yl)-3-methoxypicolinate

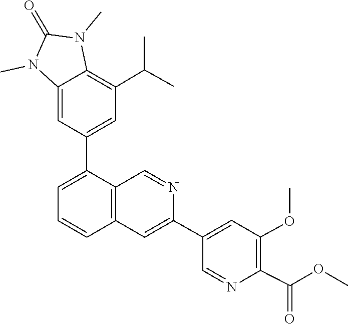

To a solution of 6-bromo-4-isopropyl-1,3-dimethyl-1,3-dihydro-2H-benzo[d]imidazol-2-one (81 mg, 286 μmol, 1 eq.; Intermediate 2, Step B) in 1,4-dioxane (10 mL) and H₂O(0.5 mL) was added methyl 3-methoxy-5-(8-(4,4,5,5-tetramethyl-1,3,2-dioxaborolan-2-yl)isoquinolin-3-yl)picolinate (120 mg, 286 μmol, 1 eq.), K₂CO₃ (118 mg, 857 μmol, 3 eq.), and X-Phos Pd G2 (22.5 mg, 28.6 μmol, 0.1 eq.) under N₂. The mixture was stirred at 100° C. for 1 hour. The mixture was diluted with water and extracted with EtOAc (20 mL×3). The combined organic layers were washed with brine, dried over Na₂SO₄, and concentrated under reduced pressure. The residue was purified by flash column chromatography on silica gel (100-200 mesh silica gel, 0-50% EtOAc in PE) to afford methyl 5-(8-(7-isopropyl-1,3-dimethyl-2-oxo-2,3-dihydro-1H-benzo[d]imidazol-5-yl)isoquinolin-3-yl)-3-methoxypicolinate (100 mg, 67% yield) as a yellow solid.

LC-MS (ESI): mass calcd. for C₂₉H₂₈N₄O₄, 496.2; m/z found, 497.6 [M+H]⁺.

### Step F: 5-(8-(7-Isopropy-1,3-dimethyl-2-oxo-2,3-dihydro-]H-benzo[d]imidazol-5-yl)isoquinolin-3-yl)-3-methoxypicolinic acid

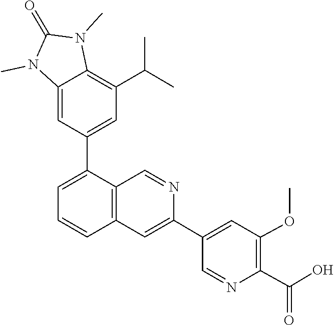 [SMILES: COc1cc(-c2cc3cccc(-c4cc(C(C)C)c5c(c4)n(C)c(=O)n5C)c3cn2)cnc1C(=O)O]

To a solution of methyl 5-(8-(7-isopropyl-1,3-dimethyl-2-oxo-2,3-dihydro-1H-benzo[d]imidazol-5-yl)isoquinolin-3-yl)-3-methoxypicolinate (50 mg, 101 μmol, 1 eq.) in THF (5 mL) and water (3 mL) was added LiGH (9 mg, 201 μmol, 2 eq.). The mixture was stirred at 25° C. for 2 hours. The mixture was concentrated to afford 5-(8-(7-isopropyl-1,3-dimethyl-2-oxo-2,3-dihydro-1H-benzo[d]imidazol-5-yl)isoquinolin-3-yl)-3-methoxypicolinic acid (40 mg, 78% yield) as a yellow solid.

LC-MS (ESI): mass calcd. for C₂₈H₂₆N₄O₄, 482.2; m/z found, 483.5 [M+H]⁺.

### Step G: N-(3-(3-(1-(Hydroxymethyl)-2,6-dioxopiperidin-3-yl)-1-methyl-1H-indazol-5-yl)prop-2-yn-1-yl)-5-(8-(7-isopropyl-1,3-dimethyl-2-oxo-2,3-dihydro-]H-benzo[d]imidazol-5-yl)isoquinolin-3-yl)-3-methoxypicolinamide

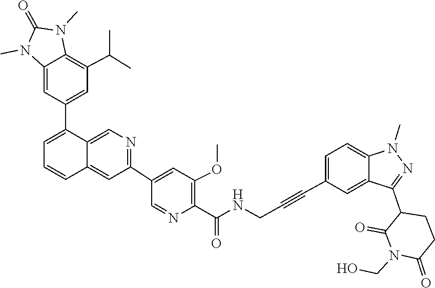 [SMILES: COc1cc(-c2cc3cccc(-c4cc(C(C)C)c5c(c4)n(C)c(=O)n5C)c3cn2)cnc1C(=O)NCC#Cc1ccc2c(c1)c(C1CCC(=O)N(CO)C1=O)nn2C]

To a solution of 3-(5-(3-aminoprop-1-yn-1-yl)-1-methyl-1H-indazol-3-yl)-1-(hydroxymethyl)piperidine-2,6-dione (27 mg, 82.9 μmol, 1 eq.; Example A5, Step F) in DMF (5 mL) was added 5-(8-(7-isopropyl-1,3-dimethyl-2-oxo-2,3-dihydro-1H-benzo[d]imidazol-5-yl)isoquinolin-3-yl)-3-methoxypicolinic acid (40 mg, 82.9 μmol, 1 eq.), DIPEA (60 μL, 332 μmol, 4 eq.), and HATU (63 mg, 166 μmol, 2 eq.). The mixture was stirred at 0° C. for 30 mins. The resulting solution was diluted with 10 mL of H₂O and extracted with EtOAc (20 mL×3). The combined organic layers were washed with brine, dried over Na₂SO₄, and concentrated under reduced pressure. The residue was purified by flash chromatography (100-200 mesh silica gel, 0-10% MeOH in DCM) to afford N-(3-(3-(1-(hydroxymethyl)-2,6-dioxopiperidin-3-yl)-1-methyl-1H-indazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)-3-methoxypicolinamide (20.0 mg, 29% yield) as a yellow solid.

LC-MS (ESI): mass calcd. for C₄₅H₄₂N₈O₆, 790.3; m/z found, 791.3 [M+H]⁺.

### Step H: N-(3-(3-(2,6-Dioxopiperidin-3-yl)-]-methyl-1H-indazol-5-yl)prop-2-yn-1-yl)-5-(8-(7-isopropyl-],3-dimethyl-2-oxo-2,3-dihydro-]H-benzo[d]imidazol-5-yl)isoquinolin-3-yl)-3-methoxypicolinamide

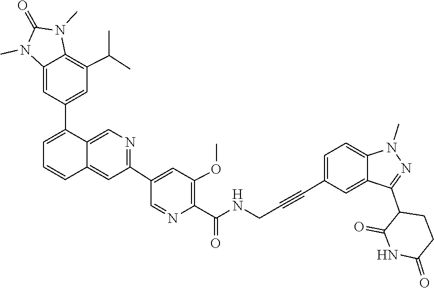 [SMILES: COc1cc(-c2cc3cccc(-c4cc(C(C)C)c5c(c4)n(C)c(=O)n5C)c3cn2)cnc1C(=O)NCC#Cc1ccc2c(c1)c(C1CCC(=O)NC1=O)nn2C]

A mixture of N-(3-(3-(1-(hydroxymethyl)-2,6-dioxopiperidin-3-yl)-1-methyl-1H-indazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)-3-methoxypicolinamide (20.0 mg, 25.3 μmol, 1 eq.) in MeCN (5 mL) and NH₄OH (0.1 mL) was stirred at 25° C. for 30 mins. The mixture was treated with TFA until a sample was measured to be pH=4. After the mixture was neutralized, it was directly concentrated under reduced pressure. The mixture was purified by Prep-HPLC (Triant C18 5 μm column, 0-70% MeCN in H₂O with 0.1% FA) to afford N-(3-(3-(2,6-dioxopiperidin-3-yl)-1-methyl-1H-indazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)-3-methoxypicolinamide (5.7 mg, 27% yield) as a yellow solid.

LC-MS (ESI): mass calcd. for C₄₄H₄₀N₈O₅, 760.3; m/z found, 761.3 [M+H]⁺.

¹H NMR (400 MHz, DMSO-d₆) δ 10.89 (s, 1H), 9.39 (s, 1H), 9.02 (d, J=1.6 Hz, 1H), 8.93 (t, J=5.6 Hz, 1H), 8.77 (s, 1H), 8.31 (d, J=1.6 Hz, 1H), 8.09 (d, J=8.4 Hz, 1H), 7.93-7.88 (m, 2H), 7.67 (d, J=7.1 Hz, 1H), 7.63 (d, J=8.8 Hz, 1H), 7.45-7.41 (m, 1H), 7.28 (d, J=1.6 Hz, 1H), 7.21 (d, J=1.6 Hz, 1H), 4.46-4.36 (m, 1H), 4.33 (d, J=5.6 Hz, 2H), 4.01-3.98 (m, 6H), 3.78-3.69 (m, 1H), 3.66 (s, 3H), 3.39 (s, 3H), 2.68-2.56 (m, 2H), 2.43-2.37 (m, 1H), 2.19-2.12 (m, 1H), 1.35 (d, J=6.7 Hz, 6H).

### Example A31. N-(3-(3-(Difluoromethyl)-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: 6-Bromo-3-(difluoromethyl)-1H-indazole

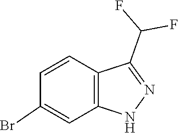 [SMILES: FC(F)c1n[nH]c2cc(Br)ccc12]

To a solution of 6-bromo-1H-indazole-3-carbaldehyde (2 g, 8.9 mmol, 1 eq.) in DCM (30 mL) was added DAST (3.5 mL, 26.7 mmol, 3 eq.) at 0° C. The mixture was stirred at 25° C. for 2 hours. The mixture was quenched with sat. aq. NaHCO₃ solution and extracted with DCM (70 mL×3). The combined organic layers were washed with brine, dried over Na₂SO₄, and concentrated under reduced pressure. The residue was purified by flash column chromatography on silica gel (100-200 mesh silica gel, 0-50% EtOAc in PE) to give 6-bromo-3-(difluoromethyl)-1H-indazole (720 mg, 31% yield) as a yellow solid.

LC-MS (ESI): mass calcd. for C₈H₅BrF₂N₂, 246.0/248.0; m/z found, 247/249 [M+H]⁺.

### Step B to E: N-(3-(3-(Difluoromethyl)-1-(2,6-dioxopiperidin-3-yl)-]H-indazol-6-yl)prop-2-yn-1-yl)-5-(8-(7-isopropyl-1,3-dimethyl-2-oxo-2,3-dihydro-]H-benzo[d]imidazol-5-yl)isoquinolin-3-yl)picolinamide

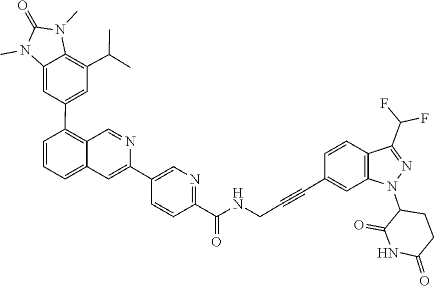 [SMILES: CC(C)c1cc(-c2cccc3cc(-c4ccc(C(=O)NCC#Cc5ccc6c(C(F)F)nn(C7CCC(=O)NC7=O)c6c5)nc4)ncc23)cc2c1n(C)c(=O)n2C]

N-(3-(3-(Difluoromethyl)-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-3-(difluoromethyl)-1H-indazole according to the procedures described in Example A1, Steps A-D. The title compound (12.4 mg, 6% yield over 4 steps) was obtained as a yellow solid.

LC-MS (ESI): mass calcd. for C₄₃H₃₆F₂N₈O₄, 766.3; m/z found, 767 [M+H]⁺.

¹H NMR (400 MHz, DMSO-d₆) δ 11.28-10.96 (m, 1H), 9.50 (s, 1H), 9.44-9.35 (m, 2H), 8.84-8.75 (m, 2H), 8.23 (d, J=8.2 Hz, 1H), 8.10 (d, J=7.9 Hz, 1H), 7.96-7.89 (m, 2H), 7.85 (d, J=8.6 Hz, 1H), 7.69 (d, J=7.3 Hz, 1H), 7.50-7.21 (m, 4H), 6.00 (s, 1H), 4.42 (d, J=6.0 Hz, 2H), 3.79-3.70 (m, 1H), 3.66 (s, 3H), 3.39 (s, 3H), 2.80-2.73 (m, 2H), 2.02-1.98 (m, 2H), 1.35 (d, J=6.8 Hz, 6H).

### Example A32. N-(3-(7-((2,6-Dioxopiperidin-3-yl)amino)-2,3-dihydrobenzofuran-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: tert-Butyl (5-bromo-2,3-dihydrobenzofuran-7-yl)carbamate

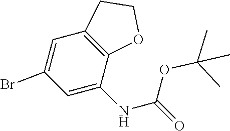 [SMILES: CC(C)(C)OC(=O)Nc1cc(Br)cc2c1OCC2]

To a solution of 5-bromo-2,3-dihydrobenzofuran-7-carboxylic acid (890 mg, 3.7 mmol, 1.0 eq.) in t-BuOH (15 mL) and toluene (15 mL) was added DIPEA (702 μL, 4.03 mmol, 1.1 eq.) and DPPA (948 μL, 4.4 mmol, 1.2 eq.). The mixture was stirred at 110° C. for 18 hours under N₂ atmosphere. The mixture was poured into EtOAc (40 mL) and filtered. The filter cake was washed with additional EtOAc (40 mL). The combined filtrates were washed with brine (40 mL), dried over Na₂SO₄, and concentrated under reduced pressure. The residue was purified by flash column chromatography on silica gel (100-200 mesh silica gel, 0-10% DCM in MeOH) to give tert-butyl (5-bromo-2,3-dihydrobenzofuran-7-yl)carbamate (850 mg, 74% yield) as a yellow solid.

LC-MS (ESI): mass calcd. for C₁₃H₁₆BrNO₃, 313.0/315.0; m/z found, 258.0/260.0 [M−tBu+H]⁺.

### Step B: 5-Bromo-2,3-dihydrobenzofuran-7-amine

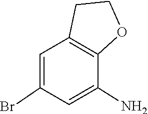 [SMILES: Nc1cc(Br)cc2c1OCC2]

To a solution of tert-butyl (5-bromo-2,3-dihydrobenzofuran-7-yl)carbamate (250 mg, 796 μmol, 1.0 eq.) in DCM (10 mL) was added TFA (2 mL) under N₂ atmosphere. The reaction was stirred for 30 minutes at room temperature. The mixture was concentrated under vacuum to give 5-bromo-2,3-dihydrobenzofuran-7-amine (150 mg, crude) as a yellow solid.

LC-MS (ESI): mass calcd. for CsHsBrNO, 213.0/215.0; m/z found, 214/216 [M+H]⁺.

### Step C: 3-((5-Bromo-2,3-dihydrobenzofuran-7-yl)amino)piperidine-2,6-dione

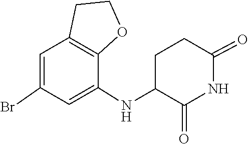 [SMILES: O=C1CCC(Nc2cc(Br)cc3c2OCC3)C(=O)N1]

A sample of 5-bromo-2,3-dihydrobenzofuran-7-amine (280 mg, 1.3 mmol, 1.0 eq.) was suspended in neat DIPEA (456 μL, 2.62 mmol, 2.0 eq.) and treated with 3-bromopiperidine-2,6-dione (502 mg, 2.6 mmol, 2.0 eq.) under N₂ atmosphere. The reaction was stirred for 18 hours at 80° C. The resulting solution was diluted with 50 mL of H₂O and extracted with EtOAc (40 mL×3). The combined organic layers were washed with brine, dried over Na₂SO₄, and concentrated under reduced pressure. The residue was purified by flash column chromatography on silica gel (100-200 mesh silica gel, 0-50% EtOAc in PE) to give 3-((5-bromo-2,3-dihydrobenzofuran-7-yl)amino)piperidine-2,6-dione (280 mg, 66% yield) as a solid.

LC-MS (ESI): mass calcd. for C₁₃H₁₃BrN₂O₃, 324.0/326.0; m/z found, 325/327 [M+H]⁺.

### Steps D-F: N-(3-(7-((2,6-Dioxopiperidin-3-yl)amino)-2,3-dihydrobenzofuran-5-yl)prop-2-yn-1-yl)-5-(8-(7-isopropyl-1,3-dimethyl-2-oxo-2,3-dihydro-]H-benzo[d]imidazol-5-yl)isoquinolin-3-yl)picolinamide

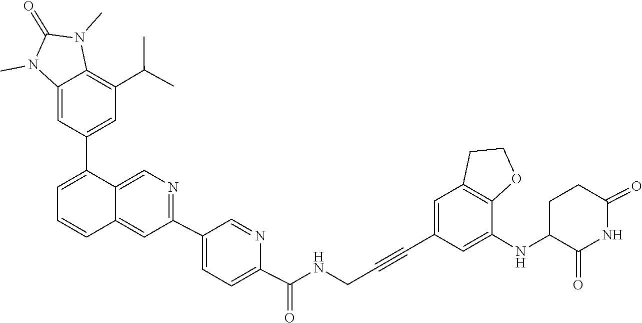 [SMILES: CC(C)c1cc(-c2cccc3cc(-c4ccc(C(=O)NCC#Cc5cc6c(c(NC7CCC(=O)NC7=O)c5)OCC6)nc4)ncc23)cc2c1n(C)c(=O)n2C]

N-(3-(7-((2,6-Dioxopiperidin-3-yl)amino)-2,3-dihydrobenzofuran-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 3-((5-bromo-2,3-dihydrobenzofuran-7-yl)amino)piperidine-2,6-dione according to the procedures described in Example A1, Steps B-D. The title compound (1.5 mg, 1% yield over 3 steps) was obtained as a white solid.

LC-MS (ESI): mass calcd. for C₄₃H₃₉N₇O₅, 733.3; m/z found, 734.2 [M+H]⁺.

¹H NMR (400 MHz, DMSO-d₆) δ 10.78 (s, 1H), 9.49 (d, J=2.0 Hz, 1H), 9.40 (s, 1H), 9.26 (t, J=5.9 Hz, 1H), 8.82-8.74 (m, 2H), 8.21 (d, J=8.2 Hz, 1H), 8.10 (d, J=8.2 Hz, 1H), 7.94-7.89 (m, 1H), 7.69 (d, J=7.0 Hz, 1H), 7.28 (s, 1H), 7.21 (s, 1H), 6.65 (s, 1H), 6.59 (s, 1H), 5.14 (d, J=7.8 Hz, 1H), 4.55 (t, J=8.8 Hz, 2H), 4.44-4.37 (m, 1H), 4.33 (d, J=6.0 Hz, 2H), 3.79-3.72 (m, 1H), 3.66 (s, 3H), 3.39 (s, 3H), 3.16-3.11 (m, 2H), 2.81-2.74 (m, 1H), 2.57-2.54 (m, 1H), 2.04-1.96 (m, 2H), 1.35 (d, J=6.8 Hz, 6H).

### Example A33. N-(4-(1-(2,6-Dioxopiperidin-3-yl)-1H-pyrazol-4-yl)benzyl)-5-(8-(7-isopropyl-1,3-dimethyl-2-oxo-2,3-dihydro-1H-benzo[d]imidazol-5-yl)isoquinolin-3-yl)picolinamide

### Step A: 3-(4-Bromo-]H-pyrazol-1-yl)piperidine-2,6-dione

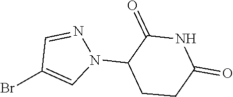 [SMILES: O=C1CCC(n2cc(Br)cn2)C(=O)N1]

To a mixture of 4-bromo-1H-pyrazole (1 g, 6.8 mmol, 1.0 eq.) in THF (10 mL) was added sodium hydride (544 mg, 13.6 mmol, 2.0 eq., w.t. =60%). The mixture was stirred at 60° C. for 1 hour. Then, 3-bromopiperidine-2,6-dione (1.96 g, 10.2 mmol, 1.2 eq.) was added, and the mixture was stirred at 60° C. for an additional 2 hours. The resulting solution was diluted with 60 mL of water and extracted with EtOAc (60 mL×3). The combined organic layers were washed with brine (50 mL), dried over Na₂SO₄, and concentrated under reduced pressure. The product was purified by flash chromatography (100-200 mesh silica gel, 0-100% EtOAc in PE) to afford 3-(4-bromo-1H-pyrazol-1-yl)piperidine-2,6-dione (550 mg, 30% yield) as a yellow solid.

LC-MS (ESI): mass calcd. for C₈H₈BrN₃O₂, 257.0/259.0; m/z found, 257.9/259.9 [M+H]⁺.

### Step B: tert-Butyl (4-(1-(2,6-dioxopiperidin-3-yl)-]H-pyrazol-4-yl)benzyl)carbamate

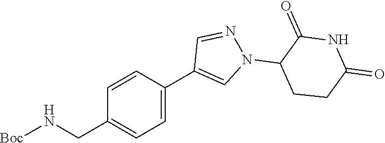

To a solution of tert-butyl (4-(4,4,5,5-tetramethyl-1,3,2-dioxaborolan-2-yl)benzyl)carbamate (123 mg, 387.5 μmol, 1.2 eq.) in 1,4-dioxane (5 mL) and water (0.5 mL) was added Pd(dppf)Cl₂ (56 mg, 77.5 μmol, 0.2 eq.), 3-(4-bromo-1H-pyrazol-1-yl)piperidine-2,6-dione (100 mg, 387.5 μmol, 1.0 eq.), and CsF (117 mg, 775.0 μmol, 2.0 eq.) at room temperature. The reaction mixture was stirred at 90° C. for 4 hours. The resulting solution was diluted with 40 mL of water and extracted with EtOAc (40 mL×3). The combined organic layers were washed with brine (40 mL), dried over Na₂SO₄, and concentrated under reduced pressure. The residue was purified by flash column chromatography on silica gel (100-200 mesh silica gel, 0-50% EtOAc in PE) to give tert-butyl (4-(1-(2,6-dioxopiperidin-3-yl)-1H-pyrazol-4-yl)benzyl)carbamate (40 mg, 27% yield) as a white solid.

LC-MS (ESI): mass calcd. for C₂₀H₂₄N₄O₄, 384.2; m/z found, 385.2 [M+H]⁺.

### Step C: 3-(4-(4-(Aminomethyl)phenyl)-]H-pyrazol-1-yl)piperidine-2,6-dione

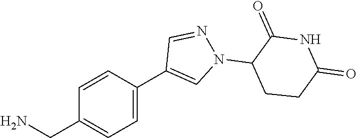

A sample of tert-butyl (4-(1-(2,6-dioxopiperidin-3-yl)-1H-pyrazol-4-yl)benzyl)carbamate (40 mg, 104 μmol, 1.0 eq.) was treated with HCl (2 mL, 4M in 1,4-dioxane) at 0° C. The reaction mixture was stirred at 0° C. for 1 hour. The reaction mixture was then concentrated to give 3-(4-(4-(aminomethyl)phenyl)-1H-pyrazol-1-yl)piperidine-2,6-dione (40 mg, crude) as a brown solid.

LC-MS (ESI): mass calcd. for C₁₅H₁₆N₄O₂, 284.1; m/z found, 285.1 [M+H]⁺.

### Step D: N-(4-(1-(2,6-Dioxopiperidin-3-yl)-]H-pyrazol-4-yl)benzyl)-5-(8-(7-isopropyl-1,3-dimethyl-2-oxo-2,3-dihydro-]H-benzo[d]imidazol-5-yl)isoquinolin-3-yl)picolinamide

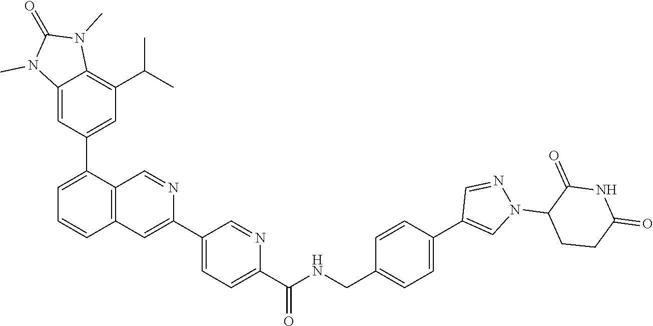 [SMILES: CC(C)c1cc(-c2cccc3cc(-c4ccc(C(=O)NCc5ccc(-c6cnn(C7CCC(=O)NC7=O)c6)cc5)nc4)ncc23)cc2c1n(C)c(=O)n2C]

To a solution of 5-(8-(7-isopropyl-1,3-dimethyl-2-oxo-2,3-dihydro-1H-benzo[d]imidazol-5-yl)isoquinolin-3-yl)picolinic acid (25 mg, 55.2 μmol, 1.0 eq.; Intermediate 2) and 3-(4-(4-(aminomethyl)phenyl)-1H-pyrazol-1-yl)piperidine-2,6-dione (40 mg, 110.4 μmol, 2.0 eq.) in DMF (2 mL) was added T3P (53 mg, 166 μmol, 3.0 eq. w.t. =50%) and DIPEA (0.1 mL, 166 μmol, 3.0 eq.) at room temperature. The reaction mixture was stirred at 50° C. for 1 hour.

The mixture was diluted with water and extracted with EtOAc (30 mL×3). The combined organic layers were washed with brine, dried over Na₂SO₄, and concentrated under reduced pressure. The residue was purified by Prep-HPLC (Triant C18 5 m column, 0-80% MeCN in H₂O with 0.1% FA) to afford N-(4-(1-(2,6-dioxopiperidin-3-yl)-1H-pyrazol-4-yl)benzyl)-5-(8-(7-isopropyl-1,3-dimethyl-2-oxo-2,3-dihydro-1H-benzo[d]imidazol-5-yl)isoquinolin-3-yl)picolinamide (16 mg, 40% yield) as a yellow solid.

LC-MS (ESI): mass calcd. for C₄₂H₃₈N₈O₄, 718.3; m/z found, 719.4 [M+H]⁺.

¹H NMR (400 MHz, DMSO-d₆) δ 11.07 (s, 1H), 9.47 (d, J=2.0 Hz, 1H), 9.42-9.37 (m, 2H), 8.80-8.74 (m, 2H), 8.22-8.19 (m, 2H), 8.09 (d, J=8.3 Hz, 1H), 7.93-7.88 (m, 2H), 7.67 (d, J=7.1 Hz, 1H), 7.56-7.53 (m, 2H), 7.37-7.33 (m, 2H), 7.28-7.19 (m, 2H), 5.37 (dd, J=12.1, 5.2 Hz, 1H), 4.52 (d, J=6.2 Hz, 2H), 3.76-3.72 (m, 1H), 3.65 (s, 3H), 3.38 (s, 3H), 2.83-2.77 (m, 1H), 2.68-2.59 (m, 2H), 2.28-2.22 (m, 1H), 1.33 (d, J=6.8 Hz, 6H).

### Example A34. N-(3-(3-((2,6-Dioxopiperidin-3-yl)amino)phenyl)prop-2-yn-1-yl)-5-((7-isopropyl-1,3-dimethyl-2-oxo-2,3-dihydro-1H-benzo[d]imidazol-5-yl)(methyl)amino)-5′-methyl-[2,3′-bipyridine]-6′-carboxamide

### Step A: Methyl 5′-methyl-5-(methylamino)-[2,3′-bipyridine]-6′-carboxylate

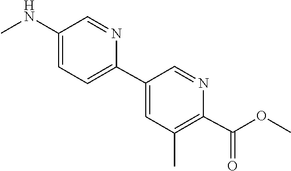

To a mixture of 6-bromo-N-methylpyridin-3-amine (525 mg, 2.8 mmol, 1.0 eq.), K₂CO₃ (1.2 g, 8.4 mmol, 3.0 eq.), and methyl 3-methyl-5-(4,4,5,5-tetramethyl-1,3,2-dioxaborolan-2-yl)picolinate (778 mg, 2.8 mmol, 1.0 eq.; Example A35, Step A) in 1,4-dioxane (10 mL) and H₂O(1 mL) was added Pd(dppf)Cl₂ (205 mg, 0.2 mmol, 0.1 eq.) at room temperature. The mixture was stirred at 90° C. for 3 hours under N₂ atmosphere. The reaction mixture was diluted with H₂O (20 mL) and extracted with EtOAc (50 mL×3). The combined organic layers were washed with brine (50 mL×2), dried over Na₂SO₄, and concentrated under reduced pressure. The residue was purified by flash column chromatography on silica gel (100-200 mesh silica gel, 0-30% EtOAc in PE) to give methyl 5′-methyl-5-(methylamino)-[2,3′-bipyridine]-6′-carboxylate (360 mg, 50% yield) as a yellow solid.

LC-MS (ESI): mass calcd. for C₁₄H₁₅N₃O₂, 257.1; m/z found, 258 [M+H]⁺.

### Step B: Methyl 5-((7-isopropyi-1,3-dimethyl-2-oxo-2,3-dihydro-]H-benzo[d]imidazol-5-yl)(methyl)amino)-5′-methyl-[2,3′-bipyridine]-6′-carboxylate

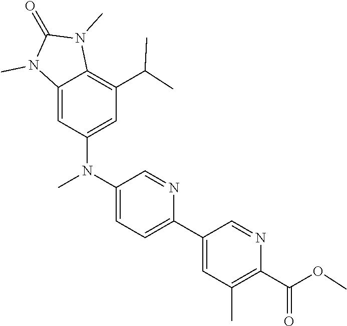

To a solution of methyl 5′-methyl-5-(methylamino)-[2,3′-bipyridine]-6′-carboxylate (200 mg, 0.7 mmol, 1.0 eq.), 6-bromo-4-isopropyl-1,3-dimethyl-1,3-dihydro-2H-benzo[d]imidazol-2-one (242 mg, 0.8 mmol, 1.1 eq.; Intermediate 2, Step B), RuPhos (72 mg, 0.2 mmol, 0.2 eq.), and Cs₂CO₃ (760 mg, 2.1 mmol, 3.0 eq.) in 1,4-dioxane (10 mL) was added RuPhos Pd G1 (113 mg, 0.2 mmol, 0.2 eq.) at room temperature. The mixture was stirred at 100° C. for 16 hours under N₂ atmosphere. The reaction mixture was diluted with H₂O(20 mL) and extracted with EtOAc (50 mL×3). The combined organic layers were washed with brine (50 mL), dried over Na₂SO₄, and concentrated under reduced pressure. The residue was purified by flash column chromatography on silica gel (100-200 mesh silica gel, 0-50% EtOAc in PE) to give methyl 5-((7-isopropyl-1,3-dimethyl-2-oxo-2,3-dihydro-1H-benzo[d]imidazol-5-yl)(methyl)amino)-5′-methyl-[2,3′-bipyridine]-6′-carboxylate (300 mg, 84% yield) as a yellow solid.

LC-MS (ESI): mass calcd. for C₂₆H₂₉N₅O₃, 459.2; m/z found, 460.5 [M+H]⁺.

### Step C: 5-((7-Isopropy-1,3-dimethyl-2-oxo-2,3-dihydro-]H-benzo[d]imidazol-5-yl)(methyl)amino)-5′-methyl-[2,3′-bipyridine]-6′-carboxylic acid

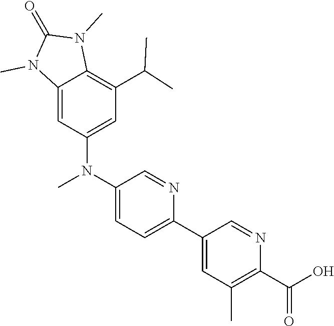 [SMILES: Cc1cc(-c2ccc(N(C)c3cc(C(C)C)c4c(c3)n(C)c(=O)n4C)cn2)cnc1C(=O)O]

To a solution of methyl 5-((7-isopropyl-1,3-dimethyl-2-oxo-2,3-dihydro-1H-benzo[d]imidazol-5-yl)(methyl)amino)-5′-methyl-[2,3′-bipyridine]-6′-carboxylate (300 mg, 0.7 mmol, 1.0 eq.) in THF (3 mL) and H₂O(1 mL) was added LiGH (55 mg, 1.4 mmol, 2.0 eq.) at room temperature. The solution was stirred at room temperature for 1 hour. The resulting solution was diluted with H₂O(20 mL) and extracted with DCM (20 mL×3). The combined organic layers were washed with brine (20 mL), dried over Na₂SO₄, and concentrated under reduced pressure to give 5-((7-isopropyl-1,3-dimethyl-2-oxo-2,3-dihydro-1H-benzo[d]imidazol-5-yl)(methyl)amino)-5′-methyl-[2,3′-bipyridine]-6′-carboxylic acid (290 mg, 99% yield) as a yellow solid.

LC-MS (ESI): mass calcd. for C₂₅H₂₇N₅O₃, 445.2; m/z found, 446 [M+H]⁺.

### Step D: N-(3-(3-((2,6-Dioxopiperidin-3-yl)amino)phenyl)prop-2-yn-1-yl)-5-((7-isopropyl-1,3-dimethyl-2-oxo-2,3-dihydro-]H-benzo[d]imidazol-5-yl)(methyl)amino)-5′-methyl-[2,3′-bipyridine]-6′-carboxamide

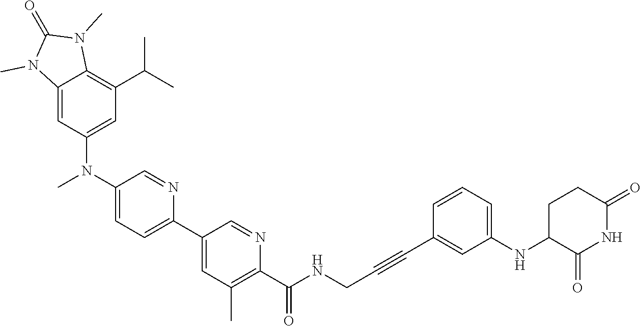 [SMILES: Cc1cc(-c2ccc(N(C)c3cc(C(C)C)c4c(c3)n(C)c(=O)n4C)cn2)cnc1C(=O)NCC#Cc1cccc(NC2CCC(=O)NC2=O)c1]

To a mixture of 5-((7-isopropyl-1,3-dimethyl-2-oxo-2,3-dihydro-1H-benzo[d]imidazol-5-yl)(methyl)amino)-5′-methyl-[2,3′-bipyridine]-6′-carboxylic acid (40 mg, 0.1 mmol, 1.0 eq.), 3-((3-(3-aminoprop-1-yn-1-yl)phenyl)amino)piperidine-2,6-dione (46 mg, 0.2 mmol, 2.0 eq.; Intermediate 6) and T3P (458 mg, 0.8 mmol, 8.0 eq., w.t. =50%) in DMF (5 mL) was added TEA (138 μL, 1.0 mmol, 10 eq.) at room temperature. The mixture was stirred at room temperature for 30 minutes under N₂ atmosphere. The reaction mixture was diluted with H₂O(20 mL) and extracted with EtOAc (20 mL×3). The combined organic layers were washed with brine (50 mL), dried over Na₂SO₄, and concentrated under reduced pressure. The residue was purified by Prep-HIPLC (C₁₈, 0-70% MeCN in H₂O with 0.1% FA) to give N-(3-(3-((2,6-dioxopiperidin-3-yl)amino)phenyl)prop-2-yn-1-yl)-5-((7-isopropyl-1,3-dimethyl-2-oxo-2,3-dihydro-1H-benzo[d]imidazol-5-yl)(methyl)amino)-5′-methyl-[2,3′-bipyridine]-6′-carboxamide (10 mg, 16% yield) as a yellow solid.

LC-MS (ESI): mass calcd. for C₃₉H₄₀N₈O₄, 684.3; m/z found, 685 [M+H]⁺.

¹H NMR (400 MHz, DMSO-d₆) δ 10.77 (s, 1H), 9.09-8.99 (m, 2H), 8.26 (s, 1H), 8.18 (d, J=2.8 Hz, 1H), 7.93 (d, J=8.8 Hz, 1H), 7.17 (dd, J=8.9, 2.9 Hz, 1H), 7.06 (t, J=7.8 Hz, 1H), 6.99 (d, J=1.9 Hz, 1H), 6.92 (d, J=1.9 Hz, 1H), 6.73-6.67 (m, 2H), 6.63 (d, J=7.5 Hz, 1H), 6.00 (d, J=7.9 Hz, 1H), 4.43-4.33 (m, 1H), 4.29 (d, J=5.9 Hz, 2H), 3.70-3.62 (m, 1H), 3.59 (s, 3H), 3.37 (s, 3H), 3.30 (s, 3H), 2.80-2.71 (m, 1H), 2.64 (s, 3H), 2.59-2.53 (m, 1H), 2.10-2.04 (m, 1H), 1.92-1.81 (m, 1H), 1.27 (d, J=6.8 Hz, 6H).

### Example A35. N-(3-(3-(2,6-Dioxopiperidin-3-yl)benzofuran-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)-3-methylpicolinamide

### Step A: Methyl 3-methyl-5-(4,4,5,5-tetramethyl-1,3,2-dioxaborolan-2-yl)picolinate

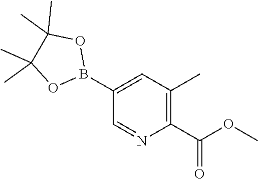

A mixture of methyl 5-bromo-3-methylpicolinate (10 g, 43.5 mmol, 1.0 eq.), 4,4,4′,4′,5,5,5′,5′-octamethyl-2,2′-bi(1,3,2-dioxaborolane) (13.2 g, 52.2 mmol, 1.2 eq.), Pd(dppf)Cl₂ (3.1 g, 4.35 mmol, 0.1 eq.), and KOAc (27.6 g, 130 mmol, 3.0 eq.) in 1,4-dioxane (100 mL) was stirred at 90° C. for 2 hours under N₂. The mixture was filtered and the filter cake was washed with EtOAc. The combined filtrates were concentrated under reduced pressure. The residue was purified by flash column chromatography on silica gel (100-200 mesh silica gel, 0-10% EtOAc in PE) to give crude product. The crude product was triturated with PE to give methyl 3-methyl-5-(4,4,5,5-tetramethyl-1,3,2-dioxaborolan-2-yl)picolinate (4.0 g, 33% yield) as a white solid.

LC-MS (ESI): mass calcd. for C₁₄H₂₀BNO₄, 277.2; m/z found, 196 [M−Pin+20H+H]⁺.

¹H NMR (400 MHz, DMSO-d₆) δ 8.63 (s, 1H), 7.99 (s, 1H), 3.87 (s, 3H), 2.59 (s, 3H), 1.33 (m, 12H).

### Step B: Methyl 5-(8-bromoisoquinolin-3-yl)-3-methylpicolinate

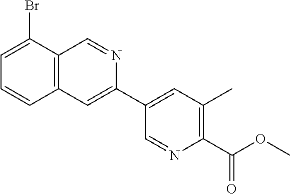 [SMILES: COC(=O)c1ncc(-c2cc3cccc(Br)c3cn2)cc1C]

A mixture of methyl 3-methyl-5-(4,4,5,5-tetramethyl-1,3,2-dioxaborolan-2-yl)picolinate (1.5 g, 5.4 mmol, 1 eq.), Pd(dppf)Cl₂ (0.4 g, 541 μmol, 0.1 eq.), 8-bromoisoquinolin-3-yl trifluoromethanesulfonate (1.9 g, 5.41 mmol, 0.1 eq.; Intermediate 1, Step C), and K₃PO₄ (3.4 g, 16.2 mmol, 3.0 eq.) in DMF (60 mL) was stirred at 70° C. for 16 hours under N₂. The mixture was poured into EtOAc (300 mL) and filtered. The filter cake was washed with additional EtOAc. The combined filtrates were washed with brine (100 mL×3), dried over Na₂SO₄, and concentrated under reduced pressure. The residue was purified by flash column chromatography on silica gel (100-200 mesh silica gel, 0-40% EtOAc in DCM) to afford methyl 5-(8-bromoisoquinolin-3-yl)-3-methylpicolinate (600 mg, 31% yield) as a yellow solid.

LC-MS (ESI): mass calcd. for C₁₇H₁₃BrN₂O₂, 356.0/358.0; m/z found, 357/359 [M+H]⁺.

### Step C: Methyl 3-methyl-5-(8-(4,4,5,5-tetramethyl-1,3,2-dioxaborolan-2-yl)isoquinolin-3-yl)picoiinate

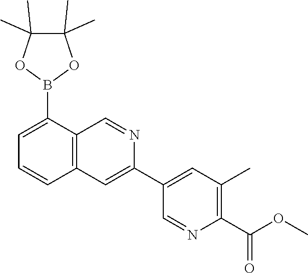

A mixture of methyl 5-(8-bromoisoquinolin-3-yl)-3-methylpicolinate (600 mg, 1.6 mmol, 1.0 eq.), Pd(dppf)Cl₂ (0.1 g, 168 μmol, 0.1 eq.), bis(pinacolato)diborane (1.3 g, 5.0 mmol, 3.0 eq.), and KOAc (1.1 g, 5.0 mmol, 3.0 eq.) in 1,4-dioxane (10 mL) was stirred at 100° C. for 3 hours under N₂. The mixture was filtered and the filter cake was washed with EtOAc. The combined filtrates were concentrated under reduced pressure. The residue was purified by flash column chromatography on silica gel (100-200 mesh silica gel, 0-40% EtOAc in PE) to give methyl 3-methyl-5-(8-(4,4,5,5-tetramethyl-1,3,2-dioxaborolan-2-yl)isoquinolin-3-yl)picolinate (500 mg, 72% yield) as a brown solid.

LC-MS (ESI): mass calcd. for C₂₃H₂₅BN₂O₄, 404.2; m/z found, 405 [M+H]⁺.

¹H NMR (400 MHz, DMSO-d₆) δ 10.07 (s, 1H), 9.32 (d, J=1.7 Hz, 1H), 8.66 (s, 1H), 8.59 (s, 1H), 8.19 (d, J=8.1 Hz, 1H), 8.12 (d, J=6.8 Hz, 1H), 7.89-7.81 (m, 1H), 3.92 (s, 3H), 2.59 (s, 3H), 1.45-1.40 (m, 12H).

### Step D: Methyl 5-(8-(7-isopropyl-1,3-dimethyl-2-oxo-2,3-dihydro-]H-benzo[d]imidazol-5-yl)isoquinolin-3-yl)-3-methylpicolinate

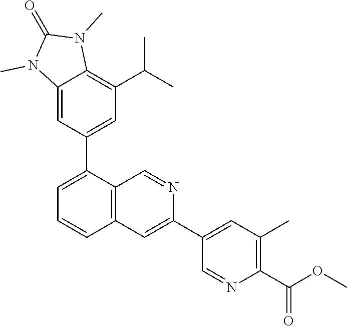

To a mixture of methyl 3-methyl-5-(8-(4,4,5,5-tetramethyl-1,3,2-dioxaborolan-2-yl)isoquinolin-3-yl)picolinate (0.8 g, 2.0 mmol, 1.0 eq.), K₂CO₃ (0.8 g, 5.9 mmol, 3.0 eq.), and 6-bromo-4-isopropyl-1,3-dimethyl-1,3-dihydro-2H-benzo[d]imidazol-2-one (560 mg, 1.9 mmol, 1.0 eq.; Intermediate 2, Step B) in 1,4-dioxane (10 mL) and H₂O(1 mL) was added X-Phos Pd G2 (155 mg, 198 μmol, 0.1 eq.) at room temperature. The mixture was stirred at 100° C. for 3 hours under N₂. The resulting solution was diluted with 30 mL of water and extracted with EtOAc (30 mL×3). The combined organic layers were washed with brine (60 mL), dried over Na₂SO₄, and concentrated under reduced pressure. The residue was purified by flash column chromatography on silica gel (100-200 mesh silica gel, 0-70% EtOAc in PE) to give methyl 5-(8-(7-isopropyl-1,3-dimethyl-2-oxo-2,3-dihydro-1H-benzo[d]imidazol-5-yl)isoquinolin-3-yl)-3-methylpicolinate (300 mg, 32% yield) as a white solid.

LC-MS (ESI): mass calcd. for C₂₉H₂₈N₄O₃, 480.2; m/z found, 481.3 [M+H]⁺.

### Step E: 5-(8-(7-Isopropy-1,3-dimethyl-2-oxo-2,3-dihydro-]H-benzo[d]imidazol-5-yl)isoquinolin-3-yl)-3-methylpicolinic acid

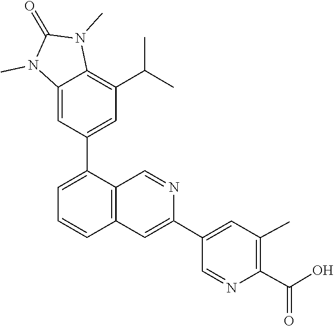 [SMILES: Cc1cc(-c2cc3cccc(-c4cc(C(C)C)c5c(c4)n(C)c(=O)n5C)c3cn2)cnc1C(=O)O]

To a mixture of methyl 5-(8-(7-isopropyl-1,3-dimethyl-2-oxo-2,3-dihydro-1H-benzo[d]imidazol-5-yl)isoquinolin-3-yl)-3-methylpicolinate (250 mg, 520 μmol, 1.0 eq.) in THE (2 mL) and water (2 mL) was added LiGH (33 mg, 780 μmol, 1.5 eq.) at room temperature. The reaction mixture was concentrated under vacuum to afford 5-(8-(7-isopropyl-1,3-dimethyl-2-oxo-2,3-dihydro-1H-benzo[d]imidazol-5-yl)isoquinolin-3-yl)-3-methylpicolinic acid (240 mg, Li salt) as a white solid.

LC-MS (ESI): mass calcd. for C₂₈H₂₆N₄O₃, 466.2; m/z found, 467.4 [M+H]⁺.

### Step F: N-(3-(3-(2,6-Dioxopiperidin-3-yl)benzofuran-5-yl)prop-2-yn-1-yl)-5-(8-(7-isopropyl-1,3-dimethyl-2-oxo-2,3-dihydro-]H-benzo[d]imidazol-5-yl)isoquinolin-3-yl)-3-methylpicolinamide

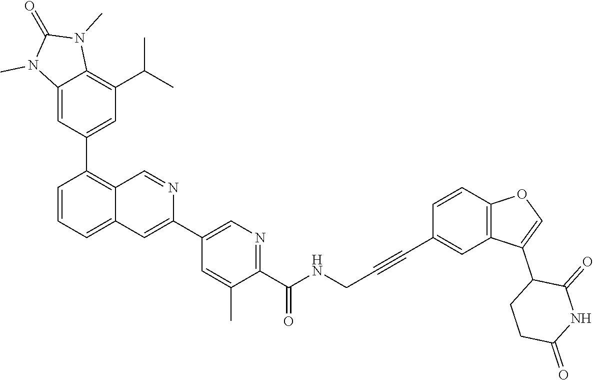 [SMILES: Cc1cc(-c2cc3cccc(-c4cc(C(C)C)c5c(c4)n(C)c(=O)n5C)c3cn2)cnc1C(=O)NCC#Cc1ccc2occ(C3CCC(=O)NC3=O)c2c1]

To a solution of 5-(8-(7-isopropyl-1,3-dimethyl-2-oxo-2,3-dihydro-1H-benzo[d]imidazol-5-yl)isoquinolin-3-yl)-3-methylpicolinic acid (47 mg, 100 μmol, 1 eq.), 3-(5-(3-aminoprop-1-yn-1-yl)benzofuran-3-yl)piperidine-2,6-dione (34 mg, 120 μmol, 1.2 eq.; as prepared in Example A23, Step D), and HATU (76 mg, 200 μmol, 2 eq.) in DMF (3 mL) was added DIPEA (69 μL, 400 μmol, 4 eq.) at 0° C. The mixture was stirred at 0° C. for 30 mins. The resulting solution was diluted with 20 mL of water and extracted with EtOAc (3×30 mL). The combined organic layers were washed with brine (30 mL), dried over Na₂SO₄, and concentrated under reduced pressure. The residue was purified by flash Prep-HPLC (Triant C18 5 m column, 0-60% MeCN in H₂O with 0.1% FA) to give N-(3-(3-(2,6-dioxopiperidin-3-yl)benzofuran-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)-3-methylpicolinamide (15.6 mg, 21% yield) as a yellow solid.

LC-MS (ESI): mass calcd. for C₄₄H₃₈N₆O₅, 730.3; m/z found, 731 [M+H]⁺.

¹H NMR (400 MHz, DMSO-d₆) δ 10.89 (s, 1H), 9.39 (s, 1H), 9.33-9.26 (m, 1H), 9.17 (t, J=5.8 Hz, 1H), 8.73 (s, 1H), 8.57 (s, 1H), 8.13-8.05 (m, 1H), 7.97 (s, 1H), 7.94-7.88 (m, 1H), 7.73 (s, 1H), 7.69-7.66 (m, 1H), 7.61-7.56 (m, 1H), 7.41-7.35 (m, 1H), 7.28 (s, 1H), 7.21 (s, 1H), 4.36 (d, J=5.8 Hz, 2H), 4.16 (dd, J=12.2, 4.8 Hz, 1H), 3.79-3.71 (m, 1H), 3.66 (s, 3H), 3.39 (s, 3H), 2.75-2.67 (m, 4H), 2.60-2.54 (m, 1H), 2.38-2.31 (m, 1H), 2.13-2.06 (m, 1H), 1.35 (d, J=6.8 Hz, 6H).

### Example A36. N-(3-(1-(2,6-Dioxopiperidin-3-yl)-3-methyl-2-oxo-2,3-dihydro-1H-benzo[d]imidazol-5-yl)prop-2-yn-1-yl)-5-(8-(7-isopropyl-1,3-dimethyl-2-oxo-2,3-dihydro-1H-benzo[djimidazol-5-yl)isoquinolin-3-yl)picolinamide

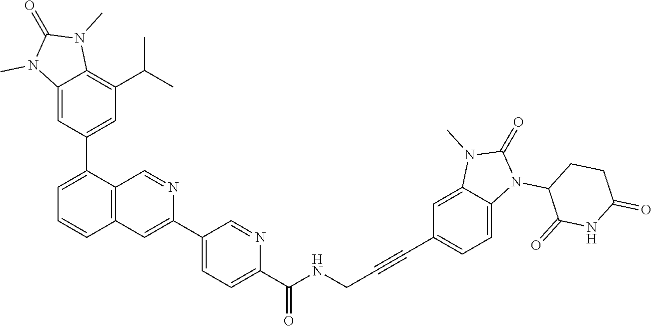 [SMILES: CC(C)c1cc(-c2cccc3cc(-c4ccc(C(=O)NCC#Cc5ccc6c(c5)n(C)c(=O)n6C5CCC(=O)NC5=O)nc4)ncc23)cc2c1n(C)c(=O)n2C]

N-(3-(1-(2,6-Dioxopiperidin-3-yl)-3-methyl-2-oxo-2,3-dihydro-1H-benzo[d]imidazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 3-(5-bromo-3-methyl-2-oxo-2,3-dihydro-1H-benzo[d]imidazol-1-yl)piperidine-2,6-dione according to the procedures described in Example A1, Steps B D. The title compound (9 mg, 6% yield over 3 steps) was obtained as a white solid.

LC-MS (ESI): mass calcd. for C₄₃H₃₈N₈O₅, 746.3; m/z found, 747.6 [M+H]⁺.

¹H NMR (400 MHz, DMSO-d₆) δ 11.11 (s, 1H), 9.50 (d, J=2.1 Hz, 1H), 9.40 (s, 1H), 9.33 (t, J=6.0 Hz, 1H), 8.80 (dd, J=8.2, 2.2 Hz, 1H), 8.77 (s, 1H), 8.23 (d, J=8.2 Hz, 1H), 8.10 (d, J=8.2 Hz, 1H), 7.94-7.89 (m, 1H), 7.69 (d, J=7.5 Hz, 1H), 7.30 (d, J=9.3 Hz, 2H), 7.21 (s, 1H), 7.16-7.10 (m, 2H), 5.38 (dd, J=12.5, 5.2 Hz, 1H), 4.38 (d, J=6.0 Hz, 2H), 3.79-3.71 (m, 1H), 3.66 (s, 3H), 3.39 (s, 3H), 3.33 (s, 3H), 2.92-2.83 (m, 1H), 2.73-2.61 (m, 2H), 2.05-1.99 (m, 1H), 1.35 (d, J=6.8 Hz, 6H).

### Example A37. 5-(7-(Difluoromethyl)-1-(7-isopropyl-1,3-dimethyl-2-oxo-2,3-dihydro-1H-benzo[d]imidazol-5-yl)-1,2,3,4-tetrahydroquinolin-6-yl)-N-(4-(1-(2,6-dioxopiperidin-3-yl)-1H-pyrazol-3-yl)benzyl)picolinamide

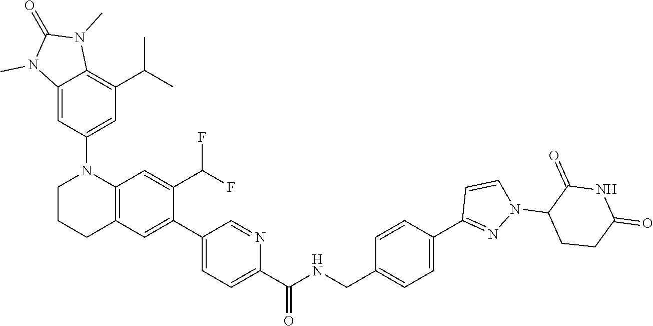 [SMILES: CC(C)c1cc(N2CCCc3cc(-c4ccc(C(=O)NCc5ccc(-c6ccn(C7CCC(=O)NC7=O)n6)cc5)nc4)c(C(F)F)cc32)cc2c1n(C)c(=O)n2C]

5-(7-(Difluoromethyl)-1-(7-isopropyl-1,3-dimethyl-2-oxo-2,3-dihydro-1H-benzo[d]imidazol-5-yl)-1,2,3,4-tetrahydroquinolin-6-yl)-N-(4-(1-(2,6-dioxopiperidin-3-yl)-1H-pyrazol-3-yl)benzyl)picolinamide was prepared from 3-bromo-1H-pyrazole according to the procedures described in Example A33, Steps A-D. In the final step, 5-(7-(difluoromethyl)-1-(7-isopropyl-1,3-dimethyl-2-oxo-2,3-dihydro-1H-benzo[d]imidazol-5-yl)-1,2,3,4-tetrahydroquinolin-6-yl)picolinic acid (Intermediate 3) was used as the coupling partner. The title compound (6.4 mg, 8% yield over 4 steps) was obtained as a yellow solid.

LC-MS (ESI): mass calcd. for C₄₃H₄₂F₂N₈O₄, 772.3; m/z found, 773.3 [M+H]⁺.

¹H NMR (400 MHz, DMSO-d₆) δ 11.07 (s, 1H), 9.40 (t, J=6.4 Hz, 1H), 8.56 (d, J=1.8 Hz, 1H), 8.11-8.08 (m, 1H), 7.90 (dd, J=8.1, 2.2 Hz, 1H), 7.84 (d, J=2.4 Hz, 1H), 7.75-7.72 (m, 2H), 7.38-7.35 (m, 2H), 7.12 (s, 1H), 7.01 (d, J=1.9 Hz, 1H), 6.95 (d, J=1.9 Hz, 1H), 6.77-6.66 (m, 3H), 5.42 (dd, J=12.1, 5.1 Hz, 1H), 4.53 (d, J=6.2 Hz, 2H), 3.70-3.66 (m, 2H), 3.66-3.63 (m, 1H), 3.59 (s, 3H), 3.32 (s, 3H), 2.94-2.90 (m, 2H), 2.84-2.77 (m, 1H), 2.71-2.61 (m, 2H), 2.29-2.23 (m, 1H), 2.07-2.03 (m, 2H), 1.26 (d, J=6.8 Hz, 6H).

### Example A38. N-(3-(1-(2,6-Dioxopiperidin-3-yl)-1H-benzo[d]imidazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

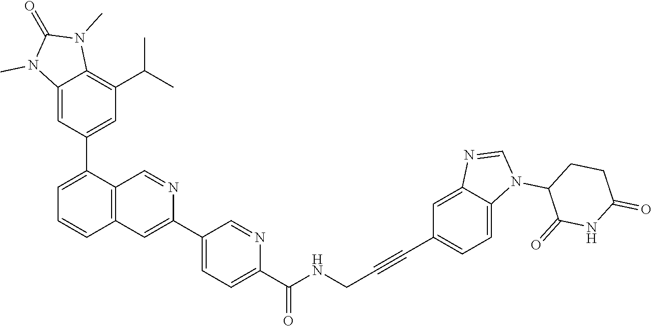 [SMILES: CC(C)c1cc(-c2cccc3cc(-c4ccc(C(=O)NCC#Cc5ccc6c(c5)ncn6C5CCC(=O)NC5=O)nc4)ncc23)cc2c1n(C)c(=O)n2C]

N-(3-(1-(2,6-Dioxopiperidin-3-yl)-1H-benzo[d]imidazol-5-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide (3.1 mg, 5% yield) was obtained during the purification described in Example A15, Step D, as a white solid.

LC-MS (ESI): mass calcd. for C₄₂H₃₆N₈O₄, 716.3; m/z found, 717.5 [M+H]⁺.

¹H NMR (400 MHz, DMSO-d₆) δ 11.20 (s, 1H), 9.50 (s, 1H), 9.42-9.38 (m, 1H), 9.34 (t, J=6.0 Hz, 1H), 8.84-8.74 (m, 2H), 8.35 (s, 1H), 8.23 (d, J=8.2 Hz, 1H), 8.10 (d, J=8.4 Hz, 1H), 7.94-7.88 (m, 1H), 7.74 (s, 1H), 7.68 (d, J=7.2 Hz, 1H), 7.56 (d, J=8.6 Hz, 1H), 7.33 (d, J=8.5 Hz, 1H), 7.29 (d, J=1.5 Hz, 1H), 7.21 (s, 1H), 5.71 (dd, J=12.9, 5.0 Hz, 1H), 4.40 (d, J=5.7 Hz, 2H), 3.79-3.72 (m, 1H), 3.66 (s, 3H), 3.39 (s, 3H), 2.94-2.79 (m, 2H), 2.68 (s, 1H), 2.29-2.20 (m, 1H), 1.35 (d, J=6.8 Hz, 6H).

### Example A39. N-(3-(7-Chloro-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide

### Step A: 4-Bromo-3-chloro-2-fluorobenzaldehyde

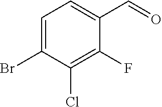 [SMILES: O=Cc1ccc(Br)c(Cl)c1F]

To a solution of 1-bromo-2-chloro-3-fluorobenzene (10 g, 47.7 mmol, 1.0 eq.) in THF (80 mL) was added LDA (31 mL, 62.1 mmol, 1.3 eq., 2.0 M in THF) at −78° C. The mixture was stirred at this temperature for 1 hour under N₂ atmosphere. Then, DMF (5.5 mL, 71.6 mmol, 1.5 eq.) was added to this solution at −78° C. The mixture was then stirred at room temperature for 30 minutes under N₂ atmosphere. The resulting solution was quenched with 100 mL of NH₄Cl and extracted with EtOAc (70 mL×3). The combined organic layers were washed with brine, dried over Na₂SO₄, and concentrated under reduced pressure. The residue was purified by flash column chromatography on silica gel (100-200 mesh silica gel, 0-10% EtOAc in PE) to give 4-bromo-3-chloro-2-fluorobenzaldehyde (4.7 g, 42% yield) as a yellow oil.

### Step B: 6-Bromo-7-chloro-1H-indazole

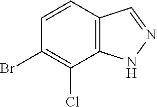 [SMILES: Clc1c(Br)ccc2cn[nH]c12]

To a solution of 4-bromo-3-chloro-2-fluorobenzaldehyde (4.7 g, 19.1 mmol, 1.0 eq.) in t-BuOH (40 mL) was added hydrazine monohydrate (4 mL, 114 mmol, 6.0 eq.) at room temperature. The mixture was stirred at 120° C. for 18 hours under N₂ atmosphere. The reaction mixture was diluted with H₂O(100 mL) and extracted with EtOAc (50 mL×3). The combined organic layers were washed with brine (50 mL), dried over Na₂SO₄, and concentrated under reduced pressure. The residue was purified by flash column chromatography on silica gel (100-200 mesh silica gel, 0-10% EtOAc in PE) to give 6-bromo-7-chloro-1H-indazole (1.1 g, 25% yield) as a yellow solid.

LC-MS (ESI): mass calcd. for C₇H₄BrClN₂, 229.9/231.9; m/z found, 231.3/233.1 [M+H]⁺.

Steps C—F: N-(3-(7-Chloro-]-(2,6-dioxopiperidin-3-yl)-]H-indazol-6-yl)prop-2-yn-1-yl)-5-(8-(7-isopropyl-1,3-dimethyl-2-oxo-2,3-dihydro-]H-benzo[d]imidazol-5-yl)isoquinolin-3-yl)picolinamide

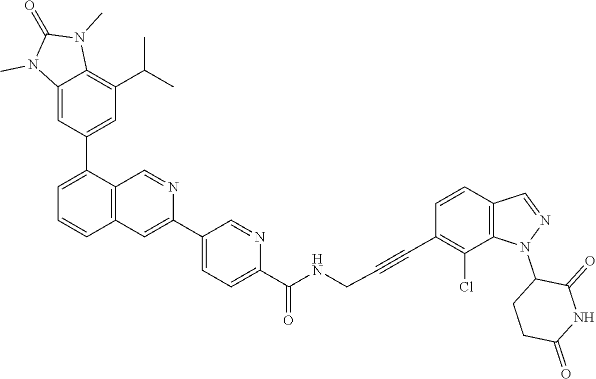 [SMILES: CC(C)c1cc(-c2cccc3cc(-c4ccc(C(=O)NCC#Cc5ccc6cnn(C7CCC(=O)NC7=O)c6c5Cl)nc4)ncc23)cc2c1n(C)c(=O)n2C]

N-(3-(7-Chloro-1-(2,6-dioxopiperidin-3-yl)-1H-indazol-6-yl)prop-2-yn-1-yl)-5-(8-(7-isopropyl-1,3-dimethyl-2-oxo-2,3-dihydro-1H-benzo[d]imidazol-5-yl)isoquinolin-3-yl)picolinamide was prepared from 6-bromo-7-chloro-1H-indazole according to the procedures described in Example A1, Steps A-D. The title compound (15 mg, 2% yield over 4 steps) was obtained as a white solid.

LC-MS (ESI): mass calcd. for C₄₂H₃₅ClN₈O₄, 750.3; m/z found, 751.3 [M+H]⁺.

¹H NMR (400 MHz, DMSO-d₆) δ 11.09 (s, 1H), 9.51-9.47 (m, 1H), 9.44-9.38 (m, 2H), 8.82-8.74 (m, 2H), 8.28 (s, 1H), 8.23 (d, J=8.2 Hz, 1H), 8.12-8.07 (m, 1H), 7.91 (t, J=7.7 Hz, 1H), 7.78 (d, J=8.3 Hz, 1H), 7.70-7.66 (m, 1H), 7.33-7.27 (m, 2H), 7.21 (s, 1H), 6.35 (s, 1H), 4.47 (d, J=5.8 Hz, 2H), 3.79-3.72 (m, 1H), 3.66 (s, 3H), 3.39-3.38 (m, 3H), 3.04-2.94 (m, 1H), 2.87-2.77 (m, 1H), 2.71-2.65 (m, 1H), 2.45-2.37 (m, 1H), 1.34 (d, J=6.8 Hz, 6H).

### Example A173. 5-(5-(1,3-dimethyl-2-oxo-2,3-dihydro-1H-benzo[d]imidazol-5-yl)-6,7-dihydr o-5H-pyrimido[4,5-b][1,4]oxazin-2-yl)-N-(3-(4-(2,6-dioxopiperidin-3-yl)benzofuran-2-yl)pr op-2-yn-1-yl)-3-methylpicolinamide

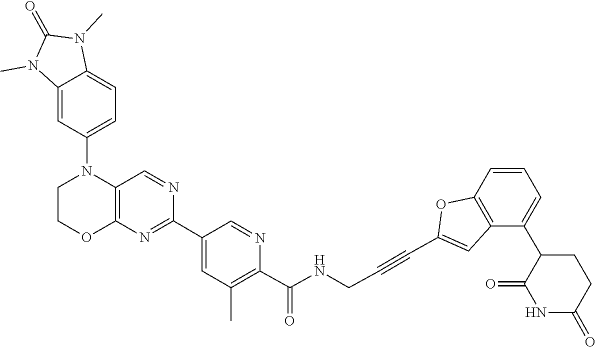 [SMILES: Cc1cc(-c2ncc3c(n2)OCCN3c2ccc3c(c2)n(C)c(=O)n3C)cnc1C(=O)NCC#Cc1cc2c(C3CCC(=O)NC3=O)cccc2o1]

### Step A: ethyl 2-(benzofuran-4-yl)acetate

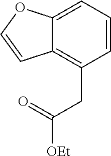 [SMILES: CCOC(=O)Cc1cccc2occc12]

To a solution of 4-bromobenzofuran (12.0 g, 60.9 mmol, 1.0 eq.), EthylPotassiumMalonate (12.4 g, 73.1 mmol, 1.2 eq.), Pd₂(allyl)C₁₂ (446 mg, 1.22 mmol, 0.02 eq.), BINAP (2.3 g, 3.65 mmol, 0.06 eq.) in 1,3,5-Trimethylbenzene (180 mL) was added DMAP (744 mg, 6.1 mmol, 0.1 eq.). The mixture was stirred at 140° C. for 14 hours under N₂ atmosphere. The resulting solution was diluted with 500 mL of H₂O and extracted with EtOAc (200 mL×3). The combined organic layers were washed with brine, dried over Na₂SO₄ and concentrated under reduced pressure. The residue was purified by flash column chromatography on silica gel (100-200 mesh silica gel, 0-5% EtOAC in PE) to give ethyl 2-(benzofuran-4-yl)acetate (6.0 g, 48% yield) as a yellow solid. LC-MS (ESI): mass calcd. for C₁₂H₁₂O₃, 204.1; m/z found, 205.2 [M+H]⁺.

### Step B: 3-(benzofuran-4-yl)piperidine-2,6-dione

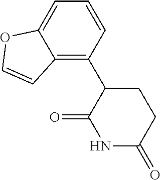 [SMILES: O=C1CCC(c2cccc3occc23)C(=O)N1]

To a solution of ethyl 2-(benzofuran-4-yl)acetate (2.8 g, 13.7 mmol, 1.0 eq.) in DMF (40 mL) was added acrylamide (2.9 g, 41.1 mmol, 3.0 eq.) and t-BuOK (1.5 g, 13.7 mmol, 1.0 eq.) at 0° C. with stirring. The mixture was stirred at 25° C. for 2 hours under N₂ atmosphere. The resulting solution was diluted with 80 mL of H₂O and extracted with EtOAc (40 mL×3). The combined organic layers were washed with brine, dried over Na₂SO₄ and concentrated under reduced pressure. The residue was purified by flash column chromatography on silica gel (100-200 mesh silica gel, 0-50% EtOAC in PE) to give 3-(benzofuran-4-yl)piperidine-2,6-dione (2.0 g, 62% yield) as a white solid._LC-MS (ESI): mass calcd. for C₁₃H₁₁NO₃, 229.1; m/z found, 230.2 [M+H]⁺.

### Step C: 3-(2-bromobenzofuran-4-yl)piperidine-2,6-dione

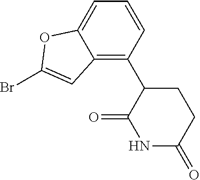 [SMILES: O=C1CCC(c2cccc3oc(Br)cc23)C(=O)N1]

To a solution of 3-(benzofuran-4-yl)piperidine-2,6-dione (4 g, 17.4 mmol, 1 eq.) in MeCN (80 mL) was added DMF (0.20 mL, 2.6 mmol, 0.15 eq.) and NBS (3.7 g, 20.9 mmol, 1.2 eq.), the mixture was stirred for 50° C. at 16 hours. The resulting solution was diluted with 200 mL of H₂O and extracted with EtOAc (50 mL×3). The combined organic layers were washed with brine, dried over Na₂SO₄ and concentrated under reduced pressure. The residue was purified by Prep-HPLC (Instrument: E-Prep LC 012; Column: Kromasil EternityXT C18 21.2*250 mm, 10-; phase A: H₂O(0.1% FA), phase B: MeCN) to give 3-(2-bromobenzofuran-4-yl)piperidine-2,6-dione (800 mg, 15% yield) as a yellow solid. 2.5 g of 3-(benzofuran-4-yl)piperidine-2,6-dione was recycled. LC-MS (ESI): mass calcd. for C₁₃H₁₀BrNO₃, 306.98/308.98; m/z found, 308.0/310.0 [M+H]⁺.

### Step D: tert-butyl (3-(4-(2,6-dioxopiperidin-3-yl)benzofuran-2-yl)prop-2-yn-1-yl)carbamate

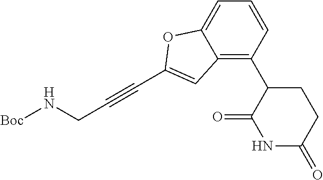 [SMILES: CC(C)(C)OC(=O)NCC#Cc1cc2c(C3CCC(=O)NC3=O)cccc2o1]

To a solution of 3-(2-bromobenzofuran-4-yl)piperidine-2,6-dione (400 mg, 1.30 mmol, 1.0 eq.) in DMF (8 mL) was added tert-butyl prop-2-yn-1-ylcarbamate (403 mg, 2.60 mmol, 2.0 eq.), Pd(PPh₃)₂C₁₂ (91 mg, 130 μmol, 0.1 eq.), CuI (25 mg, 130 μmol, 0.1 eq.), TEA (1.8 mL, 13.0 mmol, 10.0 eq.) at 25° C. The mixture was then stirred at 80° C. for 5 hours under N₂. The resulting solution was diluted with 30 mL of H₂O and extracted with EtOAc (20 mL×3). The combined organic layers were washed with brine, dried over Na₂SO₄ and concentrated under reduced pressure. The residue was purified by flash column chromatography on silica gel (100-200 mesh silica gel, 0-40% EtOAC in PE) to give tert-butyl (3-(4-(2,6-dioxopiperidin-3-yl)benzofuran-2-yl)prop-2-yn-1-yl)carbamate (260 mg, 52% yield) as a yellow solid.

LC-MS (ESI): mass calcd. for C₂₁H₂₂N₂O₅, 382.15; m/z found, 383.4 [M+H]⁺.

¹H NMR (400 MHz, DMSO-d₆) δ 10.89 (s, 1H), 7.51-7.42 (m, 2H), 7.37-7.33 (m, 1H), 7.27 (s, 1H), 7.13 (d, J=7.3 Hz, 1H), 4.22 (dd, J=12.2, 4.9 Hz, 1H), 4.09-4.03 (m, 2H), 2.76-2.67 (m, 1H), 2.58-2.52 (m, 1H), 2.39-2.31 (m, 1H), 2.07-2.00 (m, 1H), 1.41 (s, 9H).

### Step E: 3-(2-(3-aminoprop-1-yn-1-yl)benzofuran-4-yl)piperidine-2,6-dione

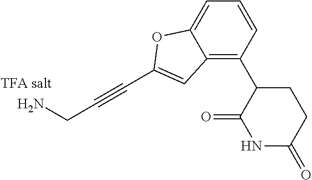 [SMILES: NCC#Cc1cc2c(C3CCC(=O)NC3=O)cccc2o1]

A solution of tert-butyl (3-(4-(2,6-dioxopiperidin-3-yl)benzofuran-2-yl)prop-2-yn-1-yl)carbamate (220 mg, 575 μmol, 1.0 eq.) in DCM (5 mL) added TFA (1 mL) at room temperature. The mixture was stirred at 25° C. for 1 hour. The residue was concentrated under reduced pressure to give 3-(2-(3-aminoprop-1-yn-1-yl)benzofuran-4-yl)piperidine-2,6-dione (220 mg, crude) as an yellow oil which was directly used to next step without purification.

LC-MS (ESI): mass calcd. for C₁₆H₁₄N₂O₃, 282.10; m/z found, 283.1 [M+H]⁺.

### Step F: methyl 2-((2-chloro-5-nitropyrimidin-4-yl)oxy)acetate

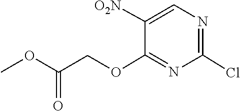 [SMILES: COC(=O)COc1nc(Cl)ncc1[N+](=O)[O-]]

To a solution of 2,4-dichloro-5-nitropyrimidine (40 g, 206 mmol, 1.0 eq.) in THE (400 mL) was added K₂CO₃ (57 g, 412 mmol, 2.0 eq.) and methyl 2-hydroxyacetate (20.4 g, 227 mmol, 1.1 eq.). The reaction mixture was stirred at 25° C. for 16 hours. The reaction was diluted with water and extracted with EtOAc (100 mL×3). The combined organic layers were washed with brine (100 mL×3), dried over Na₂SO₄ and concentrated under reduced pressure. The residue was purified by silica gel chromatography (100-200 mesh silica gel, 0-50% EtOAc in PE) to give methyl 2-((2-chloro-5-nitropyrimidin-4-yl)oxy)acetate (11 g, 22% yield) as a yellow solid.

LC-MS (ESI): mass calcd. for C₇H₆ClN₃O₅, 247; m/z found, 248 [M+H]⁺.

### Step G: 2-chloro-5H-pyrimido[4,5-b][1,4]oxazin-6(7H)-one

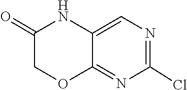 [SMILES: O=C1COc2nc(Cl)ncc2N1]

To a solution of methyl 2-((2-chloro-5-nitropyrimidin-4-yl)oxy)acetate (10 g, 40.4 mmol, 1.0 eq.), Fe (2.3 g, 40.4 mmol, 1.Oeq.) in AcOH (10 mL). The reaction mixture was stirred at 80° C. for 2 hours. The reaction mixture was poured into water and extracted with EtOAc (30 ml×3). The organic layers were washed with brine, dried Na₂SO₄ and concentrated. The residue was purified by column chromatography on silica gel eluted with PE: EtOAc (100:1˜1:1) to give 2-chloro-5H-pyrimido[4,5-b][1,4]oxazin-6(7H)-one (3.5 g, 47% yield) as a yellow solid.

LC-MS (ESI): mass calcd. for C₆H₄ClN₃O₂, 185; m/z found, 186 [M+H]⁺.

### Step H: 2-chloro-6,7-dihydro-5H-pyrimido[4,5-b][1,4]oxazine

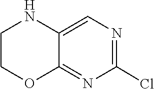 [SMILES: Clc1ncc2c(n1)OCCN2]

To a solution of 2-chloro-5H-pyrimido[4,5-b][1,4]oxazin-6(7H)-one (3.5 g, 18.9 mmol, 1 eq.) in THF (30 mL) was added BH3.THF (5.7 mL, 56.6 mmol, 3.0 eq., 10 M in THF). The reaction mixture was stirred at 40° C. for 1 hour. The reaction mixture was poured into water and extracted with EtOAc (30 ml×3). The organic layers were washed with brine, dried Na₂SO₄ and concentrated. The residue was purified by column chromatography on silica gel eluted with PE:EtOAc (100:1˜1:1) to give 2-chloro-6,7-dihydro-5H-pyrimido[4,5-b][1,4]oxazine (1.3 g, 40% yield) as a yellow solid. LC-MS (ESI): mass calcd. for C₆H₆ClN₃O, 171; m/z found, 172 [M+H]⁺. ¹H NMR (400 MHz, DMSO-d₆) δ 7.80 (s, 1H), 6.40 (s, 1H), 4.48-4.24 (m, 2H), 3.32-3.28 (m, 2H).

### Step I: methyl 5-(6,7-dihydro-5H-pyrimido[4,5-b][1,4]oxazin-2-yl)-3-methylpicolinate

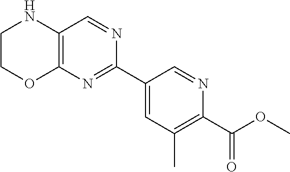 [SMILES: COC(=O)c1ncc(-c2ncc3c(n2)OCCN3)cc1C]

A mixture of 2-chloro-6,7-dihydro-5H-pyrimido[4,5-b][1,4]oxazine (1.0 g, 5.8 mmol, 1.0 eq.), methyl 3-methyl-5-(4,4,5,5-tetramethyl-1,3,2-dioxaborolan-2-yl)picolinate (3.2 g, 11.6 mmol, 2.0 eq,.), Pd(dtbpf)Cl₂ (379 mg, 583 μmol, 0.1 eq.) and K₃PO₄ (3.7 g, 17.4 mmol, 3.0 eq.) in 1,4-Dioxane (20 mL) and H₂O(1.0 mL) was stirred at 100° C. for 1 hour under N₂ atmosphere. The resulting solution was diluted with 100 mL of water and extracted with EtOAc (100 mL×3). The combined organic layers were washed with brine (100 mL), dried over Na₂SO₄ and concentrated under reduced pressure. The residue was purified by flash column chromatography on silica gel (100-200 mesh silica gel, 0-100% EtOAc in PE) to give methyl 5-(6,7-dihydro-5H-pyrimido[4,5-b][1,4]oxazin-2-yl)-3-methylpicolinate (1.0 g, 60% yield) as a yellow solid. LC-MS (ESI): mass calcd. for C₁₄H₁₄N₄O₃, 286.1; m/z found, 287.1 [M+H]⁺.

### Step J: methyl 5-(5-(1,3-dimethyl-2-oxo-2,3-dihydro-1H-benzo[d]imidazol-5-yl)-6,7-dihydro-5H-pyrimido[4,5-b][1,4]oxazin-2-yl)-3-methylpicolinate

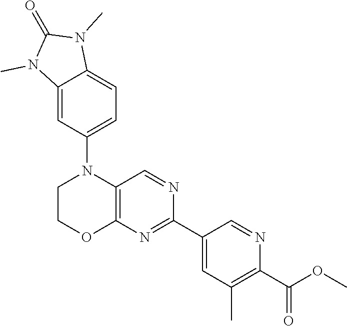

A mixture of methyl 5-(6,7-dihydro-5H-pyrimido[4,5-b][1,4]oxazin-2-yl)-3-methylpicolinate (800 mg, 2.8 mmol, 1.0 eq.), 5-bromo-1,3-dimethyl-1,3-dihydro-2H-benzo[d]imidazol-2-one (808 mg, 3.4 mmol, 1.2 eq.), Ru-Phos Pd G1 (407 mg, 0.6 mmol, 0.2 eq.), Ru-Phos (260 mg, 0.6 mmol, 0.2 eq.) and Cs₂CO₃ (2.7 g, 8.4 mmol, 3.0 eq.) in 1,4-Dioxane (10 mL) was stirred at 100° C. for 16 hours under N₂ atmosphere. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-3% MeOH in DCM) to give methyl 5-(5-(1,3-dimethyl-2-oxo-2,3-dihydro-1H-benzo[d]imidazol-5-yl)-6,7-dihydro-5H-pyrimido[4,5-b][1,4]oxazin-2-yl)-3-methylpicolinate (600 mg, 48% yield) as a yellow solid. LC-MS (ESI): mass calcd. for C₂₃H₂₂N₆O₄, 446.2; m/z found, 447.2 [M+H]⁺.

### Step K: lithium 5-(5-(1,3-dimethyl-2-oxo-2,3-dihydro-1H-benzo[d]imidazol-5-yl)-6,7-dihydro-5H-pyrimido[4,5-b][1,4]oxazin-2-yl)-3-methylpicolinate

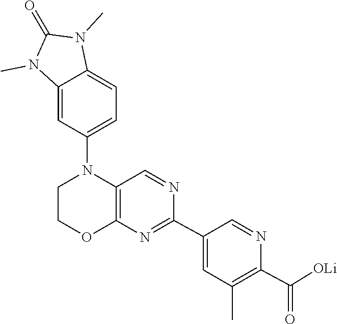

To a solution of methyl 5-(5-(1,3-dimethyl-2-oxo-2,3-dihydro-1H-benzo[d]imidazol-5-yl)-6,7-dihydro-5H-pyrimido[4,5-b][1,4]oxazin-2-yl)-3-methylpicolinate (600 mg, 1.3 mmol, 1.0 eq.) in THF (4 mL) and water (4 mL) was added lithium hydroxide (64 mg, 2.7 mmol, 2.0 eq.). The reaction mixture was stirred at 25° C. for 1 hour. Then the mixture was concentrated to afford lithium 5-(5-(1,3-dimethyl-2-oxo-2,3-dihydro-1H-benzo[d]imidazol-5-yl)-6,7-dihydro-5H-pyrimido[4,5-b][1,4]oxazin-2-yl)-3-methylpicolinate (600 mg, crude) as a Li salt. LC-MS (ESI): mass calcd. for C₂₂H₁₉LiN₆O₄, 438.2; m/z found, 432.2 [M+H]⁺. ¹H NMR (400 MHz, DMSO-d₆) δ 8.95 (s, 1H), 8.21 (s, 1H), 7.96 (S, 1H), 7.28 (s, 1H), 7.23 (d, J=6.7 Hz, 1H), 7.10 (d, J=8.1 Hz, 1H), 4.60 (s, 2H), 3.78 (s, 2H), 3.41 (s, 3H), 3.36 (s, 3H), 3.33 (s, 3H).

### Step L: 5-(5-(1,3-dimethyl-2-oxo-2,3-dihydro-1H-benzo[d]imidazol-5-yl)-6,7-dihydro-5H-pyrimido[4,5-b][1,4]oxazin-2-yl)-N-(3-(4-(2,6-dioxopiperidin-3-yl)benzofuran-2-yl)prop-2-yn-1-yl)-3-methylpicolinamide

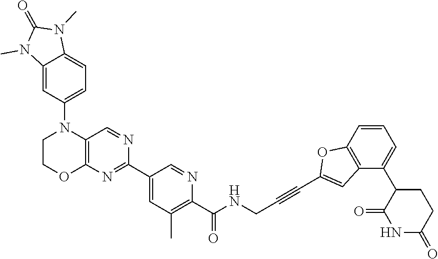 [SMILES: Cc1cc(-c2ncc3c(n2)OCCN3c2ccc3c(c2)n(C)c(=O)n3C)cnc1C(=O)NCC#Cc1cc2c(C3CCC(=O)NC3=O)cccc2o1]

A mixture of lithium 5-(5-(1,3-dimethyl-2-oxo-2,3-dihydro-1H-benzo[d]imidazol-5-yl)-6,7-dihydro-5H-pyrimido[4,5-b][1,4]oxazin-2-yl)-3-methylpicolinate (300 mg, 0.7 mmol, 1.0 eq.), 3-(2-(3-aminoprop-1-yn-1-yl)benzofuran-4-yl)piperidine-2,6-dione (215.4 mg, 0.8 mmol, 1.1 eq.), HATU (527.2 mg, 1.4 mmol, 2.0 eq.) in DMF (3 mL) was added DIPEA (0.37 mL, 2.1 mmol, 3.0 eq.) and stirred at 0 9C for 30 minutes. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-5 00 MeOH in DCM) and Prep-TIPLC (Instrument:FRC-40; Column: welch xbidge xb —C 18 5 μm 21.2*250 mm, 10 um; phase A: H₂ (0.1%(FA), phase B: MeCN) to give 5-(5-(1,3-dimethyl-2-oxo-2,3-dihydro-1H-benzo[d]imidazol-5-yl)-6,7-dihydro-5H-pyrimido[4,5-b][1,4]oxazin-2-yl)-N-(3-(4-(2,6-dioxopiperidin-3-yl)benzofuran-2-yl)prop-2-yn-1-yl)-3-methylpicolinamide (150 mg, yield 31%) as a yellow solid. LC-MS (ESI): mass calcd. for C₃₈H₃₂N₈O₆, 696.2; m/z found, 697.2 [M+H]. ¹H NMR (400 l z, DMSO-d₆) δ 10.88 (s, 1H), 9.27 (t, J=6.0 Hz, 1H), 9.21-9.14 (, 1H), 8.45-8.37 (m, 1H), 8.00 (s, 1H), 7.47 (d, J=8.3 Hz, 1H), 7.34 (t, J=7.9 Hz, 1H), 7.30 (s, 2H), 7.24 (d, J 8.3 Hz, 1H), 7.15-7.10 (s, 2H), 4.65-4.59 (m, 2H), 4.39 (d, J=5.8 Hz, 2H), 4.25-4.19 (m, 1H), 3.84-3.78 (m, 2H), 3.36 (s, 3H), 3.33 (s, 3H), 2.74-2.67 (m, 1H), 2.65 (s, 3H), 2.56-2.53 (m, 1H), 2.37-2.31 (s, 1H), 2.06-1.98 (m, 1H).

| | LC-MS: | |
|---|---|---|
| No. | [M + H]⁺ | ¹H-NMR |
| A40 | mass calcd. for | ¹H NMR (400 MHz, DMSO-d₆) |
| | C₄₁H₄₀N₈O₅, | δ 10.77 (s, 1H), 9.08- |
| | 724.3; m/z | 9.01 (m, 2H), 8.26 (s, 1H), 8.21 |
| | found, 725.8 | (d, J = 3.0 Hz, 1H), 7.94 |
| | [M + H]⁺. | (s, 1H), 7.22 (dd, J = 8.9, 3.0 Hz, |
| | | 1H), 7.10 (d, J = 2.0 Hz, |
| | | 1H), 7.06-7.01 (m, 1H), 6.76 (d, |
| | | J = 2.0 Hz, 1H), 6.73- |
| | | 6.66 (m, 2H), 6.63 (d, J = 7.6 Hz, |
| | | 1H), 6.00 (d, J = 7.9 Hz, |
| | | 1H), 5.80 (s, 1H), 4.42-4.34 (m, |
| | | 1H), 4.29 (d, J = 5.9 Hz, |
| | | 2H), 4.21 (d, J = 2.5 Hz, 2H), |
| | | 3.83 (t, J = 5.3 Hz, 2H), |
| | | 3.38-3.36 (m, 6H), 3.33 (s, 3H), |
| | | 2.79-2.70 (m, 1H), |
| | | 2.64 (s, 3H), 2.60-2.53 (m, 1H), |
| | | 2.41-2.35 (m, 2H), |
| | | 2.11-2.04 (m, 1H), 1.92-1.81 |
| | | (m, 1H). |
| A41 | mass calcd. for | ¹H NMR (400 MHz, DMSO-d₆) |
| | C₄₁H₄₁F₂N₇O₄, | δ 10.77 (s, 1H), 9.13- |
| | 733.3; | 9.07 (m, 1H), 8.41-8.36 (m, 1H), |
| | m/z found, | 7.72-7.67 (m, 1H), |
| | 734.4 | 7.26 (d, J = 8.7 Hz, 1H), 7.08-7.03 |
| | [M + H]⁺. | (m, 2H), 6.98-6.82 |
| | | (m, 4H), 6.72-6.67 (m, 2H), |
| | | 6.62 (d, J = 7.4 Hz, 1H), |
| | | 6.00 (d, J = 8.1 Hz, 1H), 4.40-4.34 |
| | | (m, 1H), 4.29 (d, J = |
| | | 5.8 Hz, 2H), 3.69-3.62 (m, 1H), |
| | | 3.59 (s, 3H), 3.32-3.30 |
| | | (m, 6H), 2.79-2.71 (m, 1H), |
| | | 2.62 (s, 3H), 2.58-2.54 (m, |
| | | 1H), 2.11-2.04 (m, 1H), 1.92-1.82 |
| | | (m, 1H), 1.26 (d, J = |
| | | 6.8 Hz, 6H). |
| A42 | mass calcd. for | ¹H NMR (400 MHz, DMSO-d₆) |
| | C₃₉H₄₀N₈O₅, | δ 10.77 (s, 1H), 9.09 (s, |
| | 700.3; m/z | 1H), 8.49 (s, 1H), 7.81 (s, 1H), |
| | found, 701.3 | 7.46-7.41 (m, 1H), 7.08- |
| | [M + H]⁺. | 7.02 (m, 1H), 7.00 (s, 1H), |
| | | 6.89 (s, 1H), 6.71-6.66 (m, |
| | | 2H), 6.63-6.60 (m, 1H), 6.00 (d, |
| | | J = 7.4 Hz, 1H), 5.71 |
| | | (d, J = 7.7 Hz, 1H), 5.46 (s, 1H), |
| | | 4.40-4.32 (m, 1H), 4.28 |
| | | (d, J = 4.7 Hz, 2H), 3.69-3.61 |
| | | (m, 1H), 3.59 (s, 3H), 3.29 |
| | | (s, 3H), 2.59 (s, 3H), 2.71-2.68 |
| | | (m, 1H), 2.61-2.57 (m, |
| | | 1H), 2.35-2.32 (m, 1H), 2.11-1.97 |
| | | (m, 4H), 1.29-1.26 |
| | | (m, 6H). |
| A43 | mass calcd. for | ¹H NMR (400 MHz, DMSO-d₆) |
| | C₄₀H₄₁N₇O₄, | δ 10.77 (s, 1H), 9.00 (t, J = |
| | 683.3; m/z | 5.9 Hz, 1H), 8.71 (d, J = 1.9 Hz, |
| | found, 684.5 | 1H), 7.96 (d, J = 1.7 |
| | [M + H]⁺. | Hz, 1H), 7.67-7.60 (m, 2H), |
| | | 7.06 (t, J = 7.9 Hz, 1H), |
| | | 6.95-6.91 (m, 1H), 6.89-6.86 |
| | | (m, 1H), 6.83 (d, J = 8.9 |
| | | Hz, 2H), 6.72-6.66 (m, 2H), |
| | | 6.64-6.60 (m, 1H), 6.00 (s, |
| | | 1H), 4.41-4.34 (m, 1H), 4.32-4.22 |
| | | (m, 2H), 3.69-3.62 |
| | | (m, 1H), 3.58 (s, 3H), 3.33-3.32 |
| | | (m, 3H), 3.30-3.29 (m, |
| | | 3H), 2.78-2.71 (m, 1H), 2.63 |
| | | (s, 3H), 2.59-2.54 (m, |
| | | 1H), 2.09-2.01 (m, 1H), 1.91-1.81 |
| | | (m, 1H), 1.27-1.25 |
| | | (m, 6H). |
| A44 | mass calcd. for | ¹H NMR (400 MHz, DMSO-d₆) |
| | C₄₀H₄₀ClN₇O₄, | δ 10.77 (s, 1H), 9.08 (t, J = |
| | 717.2; | 5.8 Hz, 1H), 8.50-8.47 (m, 1H), |
| | m/z found, | 7.77 (d, J = 1.5 Hz, |
| | 718.2 | 1H), 7.30-7.25 (m, 1H), 7.06 (s, |
| | [M + H]⁺. | 1H), 6.97 (d, J = 2.0 Hz, |
| | | 1H), 6.90 (d, J = 2.0 Hz, 1H), |
| | | 6.80 (d, J = 2.5 Hz, 1H), |
| | | 6.74-6.66 (m, 3H), 6.64-6.61 |
| | | (m, 1H), 6.00 (d, J = 7.9 |
| | | Hz, 1H), 4.41-4.34 (m, 1H), |
| | | 4.28 (d, J = 5.9 Hz, 2H), |
| | | 3.69-3.62 (m, 1H), 3.59 (s, 3H), |
| | | 3.31 (d, J = 4.5 Hz, 6H), |
| | | 2.79-2.69 (m, 1H), 2.61 (s, |
| | | 3H), 2.59-2.53 (m, 1H), |
| | | 2.10-2.04 (m, 1H), 1.92-1.81 |
| | | (m, 1H), 1.27 (d, J = 6.8 |
| | | Hz, 6H). |
| A45 | mass calcd. for | ¹H NMR (400 MHz, DMSO-d₆) |
| | C₄₁H₄₂N₈O₅, | δ 10.77 (s, 1H), 9.10- |
| | 726.3; m/z | 8.99 (m, 2H), 8.28-8.24 (m, 1H), |
| | found, 727.8 | 8.21-8.17 (m, 1H), |
| | [M + H]⁺. | 7.96-7.90 (m, 1H), 7.19 (dd, |
| | | J = 8.9, 3.0 Hz, 1H), 7.06 |
| | | (t, J = 7.9 Hz, 1H), 7.03-7.01 |
| | | (m, 1H), 6.91 (d, J = 2.0 |
| | | Hz, 1H), 6.72-6.65 (m, 2H), |
| | | 6.65-6.59 (m, 1H), 6.04- |
| | | 5.94 (m, 1H), 4.42-4.33 (m, |
| | | 1H), 4.29 (d, J = 5.9 Hz, |
| | | 2H), 3.96-3.90 (m, 2H), 3.62 |
| | | (s, 3H), 3.54-3.48 (m, |
| | | 3H), 3.37 (s, 3H), 3.31 (s, 3H), |
| | | 2.79-2.72 (m, 1H), 2.64 |
| | | (s, 3H), 2.60-2.54 (m, 1H), |
| | | 2.11-2.05 (m, 1H), 1.92- |
| | | 1.83 (m, 1H), 1.78-1.71 (m, 4H). |
| A46 | mass calcd. for | ¹H NMR (400 MHz, DMSO-d₆) |
| | C₄₁H₄₀N₈O₄, | δ 10.77 (s, 1H), 9.13 (t, J = |
| | 708.3; m/z | 5.9 Hz, 1H), 8.61 (d, J = 1.9 Hz, |
| | found, 709.3 | 1H), 7.90 (d, J = 1.6 |
| | [M + H]⁺. | Hz, 1H), 7.49 (d, J = 8.8 Hz, 1H), |
| | | 7.15 (d, J = 2.7 Hz, |
| | | 1H), 7.08-6.97 (m, 3H), 6.91 |
| | | (d, J = 1.9 Hz, 1H), 6.74- |
| | | 6.66 (m, 2H), 6.63 (d, J = 7.6 Hz, |
| | | 1H), 6.00 (d, J = 7.9 Hz, |
| | | 1H), 4.42-4.33 (m, 1H), 4.29 |
| | | (d, J = 5.8 Hz, 2H), 3.67- |
| | | 3.63 (m, 1H), 3.59 (s, 3H), 3.35 |
| | | (s, 3H), 3.31 (s, 3H), 2.80- |
| | | 2.70 (m, 1H), 2.62 (s, 3H), |
| | | 2.59-2.54 (m, 1H), 2.14- |
| | | 2.03 (m, 1H), 1.86-1.82 (m, |
| | | 1H), 1.27 (d, J = 6.8 Hz, |
| | | 6H). |
| A47 | mass calcd. for | ¹H NMR (400 MHz, DMSO-d₆) |
| | C₃₉H₃₉FN₈O₄, | δ 10.77 (s, 1H), 9.09 (t, J = |
| | 702.3; m/z | 5.9 Hz, 1H), 8.89 (s, 1H), |
| | found, 703.8 | 8.14 (s, 1H), 7.99 (t, J = 2.0 |
| | [M + H]⁺. | Hz, 1H), 7.09-7.02 (m, 3H), |
| | | 6.98-6.94 (m, 1H), 6.74- |
| | | 6.67 (m, 2H), 6.65-6.61 (m, |
| | | 1H), 6.00 (d, J = 7.9 Hz, |
| | | 1H), 4.41-4.33 (m, 1H), 4.29 (d, |
| | | J = 5.9 Hz, 2H), 3.70- |
| | | 3.62 (m, 1H), 3.60 (s, 3H), 3.38 |
| | | (s, 3H), 3.31 (s, 3H), 2.80- |
| | | 2.71 (m, 1H), 2.63 (s, 3H), |
| | | 2.60-2.53 (m, 1H), 2.11- |
| | | 2.05 (m, 1H), 1.93-1.82 (m, |
| | | 1H), 1.30-1.25 (m, 6H). |
| A48 | mass calcd. for | ¹H NMR (400 MHz, DMSO-d₆) |
| | C₄₁H₄₀F₃N₇O₄, | δ 10.77 (s, 1H), 9.11 (t, J = |
| | 751.31; | 5.9 Hz, 1H), 8.35 (s, 1H), 7.67 |
| | m/z found, | (s, 1H), 7.26 (d, J = 8.7 |
| | 752.31 | Hz, 1H), 7.05 (d, J = 7.4 Hz, |
| | [M + H]⁺. | 2H), 7.01-6.98 (m, 2H), |
| | | 6.92 (d, J = 2.0 Hz, 1H), 6.71 |
| | | (s, 1H), 6.68 (d, J = 8.1 Hz, |
| | | 1H), 6.62 (d, J = 7.5 Hz, 1H), |
| | | 5.99 (s, 1H), 4.40-4.34 (m, |
| | | 1H), 4.28 (d, J = 5.9 Hz, 2H), |
| | | 3.66 (dt, J = 13.7, 6.8 Hz, |
| | | 1H), 3.59 (s, 3H), 3.37 (s, 3H), |
| | | 3.31 (s, 3H), 2.78-2.73 |
| | | (m, 1H), 2.60 (s, 3H), 2.55-2.50 |
| | | (m, 1H), 2.11-2.05 (m, |
| | | 1H), 1.90-1.82 (m, 1H), |
| | | 1.26 (d, J = 6.8 Hz, 6H). |
| A49 | mass calcd. for | ¹H NMR (400 MHz, DMSO-d₆) |
| | C₃₉H₄₀N₈O₅, | δ 10.78 (s, 1H), 8.82 (t, J = |
| | 700.3; m/z | 5.7 Hz, 1H), 8.77-8.73 (m, 1H), |
| | found, 701.5 | 8.19 (d, J = 3.0 Hz, |
| | [M + H]⁺. | 1H), 8.03 (d, J = 1.5 Hz, 1H), |
| | | 7.99-7.93 (m, 1H), 7.18 |
| | | (dd, J = 8.9, 3.0 Hz, 1H), 7.10-7.04 |
| | | (m, 1H), 7.01-6.98 |
| | | (m, 1H), 6.92 (d, J = 2.0 Hz, |
| | | 1H), 6.74-6.67 (m, 2H), |
| | | 6.66-6.61 (m, 1H), 6.04-5.98 |
| | | (m, 1H), 4.42-4.35 (m, |
| | | 1H), 4.27 (d, J = 5.7 Hz, 2H), |
| | | 3.91 (s, 3H), 3.69-3.63 (m, |
| | | 1H), 3.59 (s, 3H), 3.37 (s, 3H), |
| | | 3.31 (s, 3H), 2.81-2.71 |
| | | (m, 1H), 2.61-2.54 (m, 1H), |
| | | 2.13-2.07 (m, 1H), 1.94- |
| | | 1.83 (m, 1H), 1.29-1.25 (m, 6H). |
| A50 | mass calcd. for | ¹H NMR (400 MHz, DMSO) |
| | C₃₈H₃₉N₉O₄, | δ 10.77 (s, 1H), 9.12-8.98 |
| | 685.31; m/z | (m, 2H), 8.91 (s, 1H), 8.27 (s, 1H), |
| | found, 686.31 | 7.90 (s, 1H), 7.13 (d, J = |
| | [M + H]⁺. | 1.8 Hz, 1H), 7.09-7.01 (m, 2H), |
| | | 6.75-6.59 (m, 3H), |
| | | 6.00 (d, J = 7.9 Hz, 1H), 4.42-4.32 |
| | | (m, 1H), 4.28 (d, J = |
| | | 5.8 Hz, 2H), 3.64 (s, 3H), 3.48 |
| | | (s, 3H), 3.32 (s, 3H), 2.82- |
| | | 2.69 (m, 1H), 2.63 (s, 3H), |
| | | 2.58-2.52 (m, 1H), 2.11- |
| | | 1.95 (m, 2H), 1.91-1.78 (m, 1H), |
| | | 1.28 (d, J = 6.8 Hz, |
| | | 6H) |
| A51 | mass calcd. for | ¹H NMR (400 MHz, DMSO-d₆) |
| | C₃₈H₃₉N₉O₄, | δ: 10.77 (s, 1H), 9.22 (s, |
| | 685.31; m/z | 1H), 9.11 (t, J = 6.0 Hz, 1H), |
| | found, 686.4 | 8.44 (s, 1H), 8.37 (s, 2H), |
| | [M + H]⁺. | 7.08-7.04 (m, 2H), 6.98 (s, |
| | | 1H), 6.72-6.67 (m, 2H), |
| | | 6.63 (d, J = 7.6 Hz, 1H), 6.00 |
| | | (d, J = 7.6 Hz, 1H), 4.38 (t, |
| | | J = 8.4 Hz, 1H), 4.29 (d, J = |
| | | 5.6 Hz, 2H), 3.69-3.63 (m, |
| | | 1H), 3.59 (s, 3H), 3.40 (s, 3H), |
| | | 3.31 (s, 3H), 2.78-2.70 |
| | | (m, 1H), 2.64 (s, 3H), 2.59-2.53 |
| | | (m, 1H), 2.10-2.05 (m, |
| | | 1H), 1.87-1.82 (m, 1H), 1.27 |
| | | (d, J = 6.8 Hz, 6H). |
| A52 | C₃₉H₃₇F₃N₈O₄, | ¹H NMR (400 MHz, DMSO-d₆) |
| | 738.3; | δ 10.77 (s, 1H), 9.12 (dd, |
| | m/z found, | J = 10.4, 3.9 Hz, 2H), 8.59 (s, 1H), |
| | 739.3 | 8.36 (s, 1H), 8.17 (d, J = |
| | [M + H]⁺. | 8.6 Hz, 1H), 7.81-7.75 (m, 1H), |
| | | 7.19 (d, J = 1.9 Hz, |
| | | 1H), 7.11-7.03 (m, 2H), 6.74-6.66 |
| | | (m, 2H), 6.63 (d, J = |
| | | 7.5 Hz, 1H), 6.01 (d, J = 7.9 Hz, |
| | | 1H), 4.42-4.33 (m, 1H), |
| | | 4.30 (d, J = 5.8 Hz, 2H), 3.73-3.64 |
| | | (m, 1H), 3.59 (s, 3H), |
| | | 3.33 (s, 3H), 2.81-2.71 (m, 1H), |
| | | 2.65 (s, 3H), 2.61-2.55 |
| | | (m, 1H), 2.12-2.05 (m, 1H), |
| | | 1.95-1.80 (m, 1H), 1.30- |
| | | 1.25 (m, 6H). |
| A53 | mass calcd. for | ¹H NMR (400 MHz, DMSO-d₆) |
| | C₄₂H₄₀N₈O₆, | δ 10.89 (s, 1H), 9.23 (d, J = |
| | 752.3; m/z | 1.7 Hz, 1H), 9.14 (t, J = 5.9 Hz, |
| | found, 753.6 | 1H), 8.44 (d, J = 1.4 |
| | [M + H]⁺. | Hz, 1H), 8.37 (s, 2H), 7.96 (s, |
| | | 1H), 7.72 (d, J = 1.2 Hz, |
| | | 1H), 7.58 (d, J = 8.5 Hz, 1H), |
| | | 7.37 (dd, J = 8.5, 1.6 Hz, |
| | | 1H), 7.08 (d, J = 2.0 Hz, 1H), |
| | | 6.99 (d, J = 2.0 Hz, 1H), |
| | | 4.34 (d, J = 5.8 Hz, 2H), |
| | | 4.19-4.13 (m, 1H), 3.96-3.91 |
| | | (m, 2H), 3.62 (s, 3H), 3.54-3.47 |
| | | (m, 3H), 3.40 (s, 3H), |
| | | 3.31 (s, 3H), 2.77-2.68 (m, 1H), |
| | | 2.65 (s, 3H), 2.61-2.54 |
| | | (m, 1H), 2.39-2.29 (m, 1H), |
| | | 2.13-2.06 (m, 1H), 1.81- |
| | | 1.72 (m, 4H). |
| A54 | mass calcd. for | ¹H NMR (400 MHz, DMSO-d₆) |
| | C₄₂H₄₀N₈O₆, | δ 10.89 (s, 1H), 9.23 (d, J = |
| | 752.3; m/z | 1.7 Hz, 1H), 9.14 (t, J = 5.9 Hz, |
| | found, 753.5 | 1H), 8.44 (d, J = 1.4 |
| | [M + H]⁺. | Hz, 1H), 8.37 (s, 2H), 7.96 (s, |
| | | 1H), 7.72 (d, J = 1.2 Hz, |
| | | 1H), 7.58 (d, J = 8.5 Hz, 1H), |
| | | 7.37 (dd, J = 8.5, 1.6 Hz, |
| | | 1H), 7.08 (d, J = 2.0 Hz, 1H), |
| | | 6.99 (d, J = 2.0 Hz, 1H), |
| | | 4.34 (d, J = 5.8 Hz, 2H), 4.19-4.13 |
| | | (m, 1H), 3.96-3.91 |
| | | (m, 2H), 3.62 (s, 3H), 3.54-3.47 |
| | | (m, 3H), 3.40 (s, 3H), |
| | | 3.31 (s, 3H), 2.77-2.68 (m, 1H), |
| | | 2.65 (s, 3H), 2.61-2.54 |
| | | (m, 1H), 2.39-2.29 (m, 1H), |
| | | 2.13-2.06 (m, 1H), 1.81- |
| | | 1.72 (m, 4H). |
| A55 | mass calcd. for | ¹H NMR (400 MHz, DMSO-d₆) |
| | C₄₂H₄₀N₈O₆, | δ 10.89 (s, 1H), 9.23 (d, J = |
| | 752.3; m/z | 1.5 Hz, 1H), 9.14 (s, 1H), |
| | found, 753.3 | 8.45 (s, 1H), 8.38 (s, 2H), |
| | [M + H]⁺. | 7.96 (s, 1H), 7.72 (d, J = 1.2 Hz, |
| | | 1H), 7.58 (d, J = 8.5 Hz, |
| | | 1H), 7.39-7.35 (m, 1H), 7.09 |
| | | (d, J = 1.9 Hz, 1H), 6.99 |
| | | (d, J = 1.9 Hz, 1H), 4.33 (d, |
| | | J = 5.8 Hz, 2H), 4.17-4.14 |
| | | (m, 1H), 3.94 (d, J = 11.0 Hz, |
| | | 2H), 3.62 (s, 3H), 3.55- |
| | | 3.48 (m, 3H), 3.40 (s, 3H), |
| | | 3.31 (s, 3H), 2.77-2.68 (m, |
| | | 1H), 2.65 (s, 3H), 2.61-2.53 |
| | | (m, 1H), 2.38-2.30 (m, |
| | | 1H), 2.12-2.05 (m, 1H), |
| | | 1.79-1.71 (m, 4H). |
| A56 | mass calcd. for | ¹H NMR (400 MHz, DMSO-d₆) |
| | C₃₉H₃₆N₈O₅, | δ 10.89 (s, 1H), 9.37 (s, |
| | 696.3; m/z | 1H), 9.15-9.08 (m, 2H), 8.98 |
| | found, 697.5 | (d, J = 2.5 Hz, 1H), 8.44 |
| | [M + H]⁺. | (dd, J = 8.8, 2.5 Hz, 1H), |
| | | 8.37-8.34 (m, 1H), 8.08 (d, J = |
| | | 8.8 Hz, 1H), 7.96 (s, 1H), |
| | | 7.74-7.71 (m, 1H), 7.59 (d, J = |
| | | 8.5 Hz, 1H), 7.38 (dd, J = 8.6, |
| | | 1.4 Hz, 1H), 6.48 (s, 1H), |
| | | 4.34 (d, J = 5.8 Hz, 2H), 4.16 |
| | | (dd, J = 12.2, 4.8 Hz, 1H), |
| | | 3.62-3.55 (m, 1H), 3.52 (s, 3H), |
| | | 3.29 (s, 3H), 2.77-2.68 |
| | | (m, 1H), 2.66 (s, 3H), 2.60-2.54 |
| | | (m, 1H), 2.38-2.30 (m, |
| | | 1H), 2.13-2.06 (m, 1H), |
| | | 1.34 (d, J = 6.6 Hz, 6H). |
| A57 | mass calcd. for | ¹H NMR (400 MHz, DMSO-d₆) |
| | C₄₅H₄₀FN₅O₆, | δ 10.88 (s, 1H), 9.32 (s, |
| | 765.8; m/z | 1H), 8.87 (s, 1H), 8.22 (d, |
| | found, 766.8 | J = 8.3 Hz, 1H), 8.15 (d, J = 8.2 |
| | [M + H]⁺. | Hz, 1H), 7.96 (s, 1H), 7.71 |
| | | (d, J = 1.6 Hz, 1H), 7.57 (d, J = |
| | | 8.5 Hz, 1H), 7.36 (dd, J = 8.5, |
| | | 1.7 Hz, 1H), 7.23 (d, J = |
| | | 6.8 Hz, 1H), 6.99 (dd, J = 16.6, |
| | | 1.6 Hz, 2H), 6.89 (d, J = |
| | | 11.5 Hz, 1H), 5.95 (s, 1H), 4.36 |
| | | (d, J = 5.9 Hz, 2H), 4.15 |
| | | (dd, J = 12.1, 4.9 Hz, 1H), |
| | | 3.85-3.65 (m, 1H), 3.61 (s, |
| | | 3H), 3.35 (s, 3H), 2.79-2.54 |
| | | (m, 2H), 2.43-2.18 (m, |
| | | 1H), 2.22-1.96 (m, 1H), 1.50 |
| | | (s, 6H), 1.30 (d, J = 6.7 Hz, |
| | | 6H). |
| A58 | mass calcd. for | ¹H NMR(400 MHz, DMSO-d₆) |
| | C₄₁H₃₇N₇O₆, | δ 10.88 (s, 1H), 9.23 (dd, |
| | 723.3; m/z | J = 12.2, 3.6 Hz, 2H), 8.51 (dd, |
| | found, 724.3 | J = 8.3, 2.0 Hz, 1H), 8.08 |
| | [M + H]⁺. | (d, J = 8.3 Hz, 1H), 7.96 (d, |
| | | J = 3.9 Hz, 2H), 7.70 (s, 1H), |
| | | 7.66 (s, 1H), 7.57 (d, J = 8.5 Hz, |
| | | 1H), 7.36 (dd, J = 8.6, |
| | | 1.4 Hz, 1H), 7.07 (d, J = 1.7 Hz, |
| | | 1H), 7.01 (d, J = 1.8 Hz, |
| | | 1H), 4.49-4.43 (m, 2H), 4.35 (d, |
| | | J = 6.0 Hz, 2H), 4.15 |
| | | (dd, J = 12.1, 4.9 Hz, 1H), |
| | | 3.81-3.77 (m, 2H), 3.69- |
| | | 3.64 (m, 1H), 3.59 (s, 3H), 3.32 |
| | | (s, 3H), 2.75-2.67 (m, |
| | | 1H), 2.59-2.54 (m, 1H), |
| | | 2.37-2.29 (m, 1H), 2.11-2.05 |
| | | (m, 1H), 1.29 (d, J = 6.8 Hz, 6H). |
| A59 | mass calcd. for | ¹H NMR (400 MHz, DMSO-d₆) |
| | C₃₉H₃₆N₈O₅, | δ 10.88 (s, 1H), 9.42- |
| | 696.8; m/z | 9.16 (m, 2H), 8.95 (d, J = 1.5 |
| | found, 697.8 | Hz, 1H), 8.51 (dd, J = 8.3, |
| | [M + H]⁺. | 2.2 Hz, 1H), 8.12 (d, J = 8.3 |
| | | Hz, 1H), 7.96 (s, 1H), 7.92 |
| | | (d, J = 1.4 Hz, 1H), 7.71 (d, |
| | | J = 1.6 Hz, 1H), 7.59 (s, 1H), |
| | | 7.37 (dd, J = 8.5, 1.6 Hz, 1H), |
| | | 7.14 (d, J = 2.1 Hz, 1H), |
| | | 7.05 (d, J = 2.1 Hz, 1H), 4.36 |
| | | (d, J = 5.9 Hz, 2H), 4.15 |
| | | (dd, J = 12.2, 4.9 Hz, 1H), |
| | | 3.72-3.61 (m, 1H), 3.61 (s, |
| | | 3H), 3.49 (s, 3H), 3.33 (s, 3H), |
| | | 2.76-2.68 (m, 1H), 2.61- |
| | | 2.57 (m, 1H), 2.40-2.28 (m, |
| | | 1H), 2.17-2.03 (m, 1H), |
| | | 1.29 (d, J = 6.8 Hz, 6H). |
| A60 | mass calcd. for | ¹H NMR (400 MHz, DMSO-d₆) |
| | C₄₀H₃₈N₈O₅, | δ 10.89 (s, 1H), 9.16- |
| | 710.3; m/z | 9.09 (m, 2H), 8.67 (d, J = 2.7 |
| | found, 711.5 | Hz, 1H), 8.38 (d, J = 1.4 Hz, |
| | [M + H]⁺. | 1H), 8.09 (d, J = 8.7 Hz, 1H), |
| | | 7.97 (s, 1H), 7.80-7.76 (m, |
| | | 1H), 7.74-7.71 (m, 1H), 7.58 |
| | | (d, J = 8.6 Hz, 1H), 7.38 |
| | | (dd, J = 8.6, 1.4 Hz, 1H), 6.79 |
| | | (s, 1H), 4.34 (d, J = 5.8 Hz, |
| | | 2H), 4.16 (dd, J = 12.2, 4.8 |
| | | Hz, 1H), 3.56-3.55 (m, 1H), |
| | | 3.52 (d, J = 3.9 Hz, 6H), 3.29 |
| | | (s, 3H), 2.77-2.68 (m, 1H), |
| | | 2.67 (s, 3H), 2.60-2.53 (m, |
| | | 1H), 2.38-2.30 (m, 1H), |
| | | 2.13-2.06 (m, 1H), 1.23 |
| | | (d, J = 6.6 Hz, 6H). |
| A61 | mass calcd. for | ¹H NMR (400 MHz, DMSO-d₆) |
| | C₄₁H₃₂N₆O₅, | δ 10.89 (s, 1H), 9.39 (s, |
| | 688.2; m/z | 1H), 9.30 (s, 1H), 9.18 (t, 1H), |
| | found, 689.2 | 8.73 (s, 1H), 8.56 (s, 1H), |
| | [M + H]⁺. | 8.09 (d, J = 8.2 Hz, 1H), 7.97 |
| | | (s, 1H), 7.91 (t, J = 7.4 Hz, |
| | | 1H), 7.73 (s, 1H), 7.67 (d, |
| | | J = 7.0 Hz, 1H), 7.59 (d, J = 8.5 |
| | | Hz, 1H), 7.44 (s, 1H), 7.41-7.33 |
| | | (m, 2H), 7.30 (d, J = 8.0 |
| | | Hz, 1H), 4.36 (d, J = 5.2 Hz, |
| | | 2H), 4.17 (d, J = 8.2 Hz, |
| | | 1H), 3.43 (s, 3H), 3.40 (s, 3H), |
| | | 2.77 (d, J = 4.5 Hz, 1H), |
| | | 2.70 (s, 3H), 2.50-2.60 (m, |
| | | 1H), 2.35 (d, J = 11.7 Hz, |
| | | 1H), 2.11 (s, 1H). |
| A62 | mass calcd. for | ¹H NMR (400 MHz, DMSO-d₆) |
| | C₄₁H₃₂N₆O₅, | δ 10.89 (s, 1H), 9.39 (s, |
| | 688; m/z | 1H), 9.30 (s, 1H), 9.18 (t, |
| | found, 689 | J = 5.9 Hz, 1H), 8.74 (s, 1H), |
| | [M + H]⁺. | 8.57 (s, 1H), 8.09 (d, J = 8.2 |
| | | Hz, 1H), 7.97 (s, 1H), 7.94- |
| | | 7.88 (m, 1H), 7.73 (s, 1H), |
| | | 7.67 (d, J = 7.0 Hz, 1H), 7.59 |
| | | (d, J = 8.6 Hz, 1H), 7.45 (s, 1H), |
| | | 7.40-7.33 (m, 2H), 7.30 |
| | | (d, J = 9.1 Hz, 1H), 4.35 (d, |
| | | J = 5.8 Hz, 2H), 4.16 (dd, J = |
| | | 12.2, 4.9 Hz, 1H), 3.43 (s, 3H), |
| | | 3.40 (s, 3H), 2.77-2.68 |
| | | (m, 4H), 2.58-2.54 (m, 1H), |
| | | 2.38-2.31 (m, 1H), 2.15- |
| | | 2.11 (m, 1H). |
| A63 | mass calcd. for | ¹H NMR (400 MHz, DMSO-d₆) |
| | C₄₁H₃₂N₆O₅, | δ 10.88 (s, 1H), 9.35 (t, J = |
| | 688.2; m/z | 6.0 Hz, 1H), 9.22 (s, 1H), 8.90 |
| | found, 689.2 | (d, J = 1.3 Hz, 1H), 8.28- |
| | [M + H]⁺. | 8.19 (m, 3H), 7.99-7.93 (m, |
| | | 2H), 7.73-7.66 (m, 2H), |
| | | 7.58 (d, J = 8.6 Hz, 1H), |
| | | 7.41-7.33 (m, 3H), 7.28-7.25 |
| | | (m, 1H), 4.39 (d, J = 5.9 Hz, |
| | | 2H), 4.16 (dd, J = 12.2, 4.8 |
| | | Hz, 1H), 3.42 (s, 3H), 3.39 |
| | | (s, 3H), 2.71 (d, J = 13.5 Hz, |
| | | 4H), 2.58-2.54 (m, 1H), 2.33 |
| | | (t, J = 10.3 Hz, 1H), 2.12- |
| | | 2.07 (m, 1H). |
| A64 | mass | ¹H NMR (400 MHz, DMSO-d₆) |
| | calcd. for | δ 10.88 (s, 1H), 9.50 (d, J = |
| | C₄₀H₂₉FN₆O₅, | 1.9 Hz, 1H), 9.40 (s, 1H), 9.35 |
| | 692.21; | (s, 1H), 8.83-8.75 (m, |
| | m/z found, | 2H), 8.23 (d, J = 8.3 Hz, 1H), |
| | 693.2 | 8.13 (d, J = 8.1 Hz, 1H), |
| | [M + H]⁺. | 7.97-7.88 (m, 2H), 7.75-7.65 |
| | | (m, 2H), 7.58 (d, J = 8.5 |
| | | Hz, 1H), 7.39-7.32 (m, 2H), |
| | | 7.24 (dd, J = 12.0, 1.1 Hz, |
| | | 1H), 4.38 (d, J = 6.0 Hz, 2H), |
| | | 4.15 (dd, J = 12.1, 5.0 Hz, |
| | | 1H), 3.56 (s, 3H), 3.40 (s, 3H), |
| | | 2.71-2.64 (m, 1H), 2.34- |
| | | 2.31 (m, 1H), 2.12-1.96 (m, 2H). |
| A65 | mass calcd. for | ¹H NMR (400 MHz, DMSO-d₆) |
| | C₄₀H₃₅D₃N₈O₅, | δ 10.88 (s, 1H), 9.23 (d, J = |
| | 713.3; | 1.8 Hz, 1H), 9.14 (t, J = 5.8 Hz, |
| | m/z found, | 1H), 8.45 (d, J = 1.6 |
| | 714.3 | Hz, 1H), 8.37 (s, 2H), 7.96 (s, 1H), |
| | [M + H]⁺. | 7.71 (s, 1H), 7.58 (d, J = |
| | | 8.5 Hz, 1H), 7.39-7.35 (m, 1H), |
| | | 7.06 (d, J = 2.0 Hz, |
| | | 1H), 6.98 (d, J = 2.0 Hz, 1H), |
| | | 4.33 (d, J = 5.8 Hz, 2H), |
| | | 4.18-4.13 (m, 1H), 3.69-3.63 |
| | | (m, 1H), 3.33 (s, 3H), |
| | | 3.31 (s, 3H), 2.74-2.68 (m, 1H), |
| | | 2.65 (s, 3H), 2.58-2.53 |
| | | (m, 1H), 2.36-2.30 (m, 1H), |
| | | 2.13-2.06 (m, 1H), 1.27 |
| | | (d, J = 6.8 Hz, 6H). |
| A66 | mass calcd. for | ¹HNMR (400 MHz, DMSO-d₄) |
| | C₄₁H₃₁FN₆O₅, | δ 10.89 (s, 1H), 9.39 (s, |
| | 706.2; m/z | 1H), 9.30 (s, 1H), 9.18 (t, |
| | found, 707.2 | J = 5.8 Hz, 1H), 8.75 (s, 1H), |
| | [M + H]⁺. | 8.57 (s, 1H), 8.11 (d, J = 8.3 Hz, |
| | | 1H), 7.97 (s, 1H), 7.94- |
| | | 7.88(m, 1H), 7.73 (s, 1H), 7.68 |
| | | (d, J = 7.1 Hz, 1H), 7.59 |
| | | (d, J = 8.6 Hz, 1H), 7.41-7.36 |
| | | (m, 1H), 7.34 (s, 1H), 7.23 |
| | | (d, J = 11.9 Hz, 1H), 4.35 (d, |
| | | J = 5.8 Hz, 2H), 4.16 (dd, J = |
| | | 12.2,4.9 Hz, 1H), 3.56 (s, 3H), |
| | | 3.40 (s, 3H), 2.75-2.65 |
| | | (m, 4H), 2.41-2.26 (m, 2H), |
| | | 2.14-2.05 (m, 1H). |
| A67 | mass calcd. for | ¹H NMR (400 MHz, DMSO-d₆) |
| | C₄₆H₄₁N₉O₅, | δ 10.89 (s, 1H), 9.81 (s, |
| | 799.3; m/z | 1H), 9.49 (s, 1H), 9.33 (s, 1H), |
| | found, 800.3 | 8.86-8.71 (m, 2H), 8.24 |
| | [M + H]⁺. | (d, J = 8.1 Hz, 1H), 8.15 (d, |
| | | J = 7.6 Hz, 1H), 7.99-7.91 |
| | | (m, 2H), 7.88 (d, J = 7.0 Hz, |
| | | 1H), 7.72 (s, 1H), 7.63 (s, |
| | | 1H), 7.58 (d, J = 8.7 Hz, 1H), |
| | | 7.37 (d, J = 8.3 Hz, 1H), |
| | | 4.39 (d, J = 5.9 Hz, 2H), |
| | | 4.16-4.12 (m, 1H), 3.93-3.84 |
| | | (m, 1H), 3.72 (s, 3H), 3.50 |
| | | (d, J = 12.4 Hz, 2H), 3.44 (s, |
| | | 3H), 3.31-3.21 (m, 3H), |
| | | 2.77-2.66 (m, 2H), 2.64-2.57 |
| | | (m, 3H), 2.54 (s, 1H), 2.36-2.31 |
| | | (m, 1H), 2.20 (s, 3H), |
| | | 2.12-2.06 (m, 1H). |
| A68 | mass calcd. for | ¹H NMR (400 MHz, DMSO-d₆) |
| | C₃₈H₃₂N₈O₆, | δ 10.88 (s, 1H), 9.36 (d, J = |
| | 696.2; m/z | 1.9 Hz, 1H), 9.32 (t, J = 6.0 |
| | found, 697.3 | Hz, 1H), 8.64 (dd, J = 8.3, |
| | [M + H]⁺. | 2.1 Hz, 1H), 8.14 (d, J = 8.3 Hz, |
| | | 1H), 8.05 (s, 1H), 7.96 (s, |
| | | 1H), 7.71 (s, 1H), 7.57 (d, J = 8.5 |
| | | Hz, 1H), 7.36 (d, J = 8.6 |
| | | Hz, 1H), 7.12 (s, 1H), 6.89 |
| | | (s, 1H), 4.63-4.59 (m, 2H), |
| | | 4.35 (d, J = 5.9 Hz, 2H), 4.15 |
| | | (m, 1H), 3.82-3.77 (m, |
| | | 2H), 3.59 (s, 3H), 3.31-3.31 |
| | | (m, 3H), 2.71-2.66 (m, |
| | | 1H), 2.60 (s, 3H), 2.59-2.56 |
| | | (m, 1H), 2.35-2.31 (m, |
| | | 1H), 2.11-2.05 (m, 1H). |
| A69 | mass calcd. for | ¹H NMR (400 MHz, DMSO-d₆) |
| | C₄₆H₄₀N₈O₆, | δ 10.90 (s, 1H), 9.85 (s, |
| | 800.3; m/z | 1H), 9.29 (d, J = 1.6 Hz, 1H), |
| | found, 801.3 | 9.17 (t, J = 5.8 Hz, 1H), |
| | [M + H]⁺. | 8.71 (s, 1H), 8.57 (d, J = 1.4 Hz, |
| | | 1H), 8.11 (d, J = 8.0 Hz, |
| | | 1H), 7.97 (s, 1H), 7.94-7.84 (m, |
| | | 2H), 7.74 (d, J = 1.1 Hz, |
| | | 1H), 7.59 (d, J = 8.5 Hz, 1H), |
| | | 7.54 (s, 1H), 7.39 (dd, J = |
| | | 8.6, 1.5 Hz, 1H), 4.43-4.34 (m, |
| | | 4H), 4.17 (dd, J = 12.2, |
| | | 4.8 Hz, 1H), 3.69 (s, 3H), 3.41 |
| | | (s, 3H), 3.26-3.21 (m, |
| | | 2H), 3.06-3.01 (m, 2H), 2.78-2.74 |
| | | (m, 1H), 2.73 (s, |
| | | 3H), 2.58-2.52 (m, 1H), |
| | | 2.40-2.28 (m, 1H), 2.14-2.04 |
| | | (m, 3H), 1.93-1.83 (m, 2H). |
| A70 | mass calcd. for | ¹H NMR (400 MHz, DMSO-d₆) |
| | C₄₁H₄₀N₈O₅, | δ 10.89 (s, 1H), 9.22 (d, J = |
| | 724.8; m/z | 1.9 Hz, 1H), 9.14 (t, J = 5.9 |
| | found, 725.8 | Hz, 1H), 8.43 (d, J = 2.0 |
| | [M + H]⁺. | Hz, 1H), 8.30 (s, 2H), 7.96 |
| | | (s, 1H), 7.72 (d, J = 1.6 Hz, |
| | | 1H), 7.58 (d, J = 8.5 Hz, 1H), |
| | | 7.37 (dd, J = 8.6, 1.7 Hz, |
| | | 1H), 7.02 (d, J = 2.0 Hz, 1H), |
| | | 6.93 (d, J = 2.1 Hz, 1H), |
| | | 4.33 (d, J = 5.9 Hz, 2H), 4.16 |
| | | (dd, J = 12.2, 4.9 Hz, 1H), |
| | | 3.84 (q, J = 7.1 Hz, 2H), 3.70-3.62 |
| | | (m, 1H), 3.60 (s, 3H), |
| | | 3.32 (s, 3H), 2.76-2.68 (m, 1H), |
| | | 2.64 (s, 3H), 2.62-2.55 |
| | | (m, 1H), 2.40-2.26 (m, 1H), |
| | | 2.20-2.03 (m, 1H), 1.27 |
| | | (d, J = 6.8 Hz, 6H), 1.20 |
| | | (t, J = 7.0 Hz, 3H). |
| A71 | mass calcd. for | ¹H NMR (400 MHz, DMSO-d₆) |
| | C₄₂H₄₂N₈O₅, | δ 10.89 (s, 1H), 9.21 (d, J = |
| | 738.3; m/z | 1.9 Hz, 1H), 9.10 (d, J = 5.8 |
| | found, 739.3 | Hz, 1H), 8.57 (d, J = 1.9 |
| | [M + H]⁺. | Hz, 1H), 8.38 (s, 2H), 7.97 |
| | | (s, 1H), 7.71 (d, J = 1.2 Hz, |
| | | 1H), 7.59 (d, J = 8.5 Hz, 1H), |
| | | 7.39-7.35 (m, 1H), 7.05 |
| | | (d, J = 2.0 Hz, 1H), 6.98 (d, |
| | | J = 2.0 Hz, 1H), 4.33 (d, J = |
| | | 5.7 Hz, 2H), 4.19-4.13 (m, |
| | | 1H), 3.89-3.80 (m, 1H), |
| | | 3.69-3.63 (m, 1H), 3.59 (s, |
| | | 3H), 3.40 (s, 3H), 3.31 (s, |
| | | 3H), 2.76-2.68 (m, 1H), |
| | | 2.60-2.54 (m, 1H), 2.36-2.29 |
| | | (m, 1H), 2.13-2.06 (m, 1H), |
| | | 1.31-1.27 (m, 6H), 1.26- |
| | | 1.24 (m, 6H). |
| A72 | mass calcd. for | ¹H NMR (400 MHz, DMSO-d₆) |
| | C₄₀H₃₃N₇O₅, | δ 10.89 (s, 1H), 9.55 (d, J = |
| | 691.8; m/z | 2.3 Hz, 1H), 9.38 (s, 1H), 9.33 |
| | found, 692.8 | (t, J = 6.0 Hz, 1H), 8.86 |
| | [M + H]⁺. | (dd, J = 8.2, 2.2 Hz, 1H), 8.65 |
| | | (s, 1H), 8.22 (d, J = 8.1 Hz, |
| | | 1H), 7.96 (s, 1H), 7.79-7.65 |
| | | (m, 2H), 7.57 (t, J = 8.3 Hz, |
| | | 2H), 7.42-7.36 (m, 2H), 7.34 |
| | | (d, J = 7.9 Hz, 1H), 7.27 |
| | | (dd, J = 8.0, 1.7 Hz, 2H), 4.39 |
| | | (d, J = 6.0 Hz, 2H), 4.16 |
| | | (dd, J = 12.4, 4.8 Hz, 1H), 3.43 |
| | | (s, 6H), 2.84 (s, 3H), 2.36- |
| | | 2.28 (m, 2H), 2.12-1.94 (m, 2H). |
| A73 | mass calcd. for | ¹H NMR (400 MHz, DMSO-d₆) |
| | C₄₀H₂₉ClN₆O₅, | δ 10.90 (s, 1H), 9.43- |
| | 708.2; | 9.40 (m, 1H), 9.39 (s, 1H), |
| | m/z found, | 9.30-9.24 (m, 1H), 8.83 (s, |
| | 709.2 | 1H), 8.80-8.77 (m, 1H), |
| | [M + H]⁺. | 8.11-8.06 (m, 1H), 7.97 (s, |
| | | 1H), 7.95-7.91 (m, 1H), |
| | | 7.76-7.72 (m, 1H), 7.71-7.68 |
| | | (m, 1H), 7.62-7.58 (m, 1H), |
| | | 7.45-7.43 (m, 1H), 7.41- |
| | | 7.37 (m, 1H), 7.37-7.33 |
| | | (m, 1H), 7.31-7.28 (m, 1H), |
| | | 4.42-4.32 (m, 2H), 4.20-4.14 |
| | | (m, 1H), 3.43 (s, 3H), |
| | | 3.40 (s, 3H), 2.77-2.67 (m, 1H), |
| | | 2.61-2.53 (m, 1H), |
| | | 2.38-2.31 (m, 1H), 2.13-2.06 (m, 1H). |
| A74 | mass | ¹H NMR (400 MHz, DMSO-d₆) |
| | calcd. for | δ 10.89 (s, 1H), 9.34- |
| | C₃₉H₃₇N₉O₅, | 9.28 (m, 1H), 9.18 (t, J = 5.7 |
| | 711.3; m/z | Hz, 1H), 8.92 (s, 2H), 8.55 |
| | found, 712.3 | (s, 1H), 7.96 (s, 1H), 7.72 |
| | [M + H]⁺. | (s, 1H), 7.59 (d, J = 8.6 Hz, |
| | | 1H), 7.38 (d, J = 8.5 Hz, 1H), |
| | | 6.91 (s, 1H), 4.34 (d, J = 5.7 |
| | | Hz, 2H), 4.20-4.12 (m, 1H), |
| | | 3.56-3.51 (m, 7H), 3.33 (s, |
| | | 3H), 2.73 (t, J = 8.7 Hz, 1H), |
| | | 2.67 (s, 3H), 2.61-2.55 (m, |
| | | 1H), 2.39-2.32 (m, 1H), |
| | | 2.14-2.04 (m, 1H), 1.22-1.19 |
| | | (m, 6H). |
| A75 | mass | ¹H NMR (400 MHz, DMSO-d₆) |
| | calcd. for | δ 10.88 (s, 1H), 9.36- |
| | C₄₄H₄₀N₆O₆, | 9.28 (m, 2H), 8.66 (dd, J = 8.2, |
| | 748.3; m/z | 2.2 Hz, 1H), 8.30 (s, 1H), |
| | found, 749.3 | 8.17-8.12 (m, 1H), 7.96 (s, 1H), |
| | [M + H]⁺. | 7.75 (s, 1H), 7.72-7.69 |
| | | (m, 1H), 7.57 (d, J = 8.6 Hz, 1H), |
| | | 7.37 (dd, J = 8.5, 1.6 |
| | | Hz, 1H), 7.05 (dd, J = 13.5, 1.5 Hz, 2H), |
| | | 5.92 (s, 1H), 4.36 |
| | | (d, J = 5.9 Hz, 2H), 4.15 (dd, J = 12.2, |
| | | 4.8 Hz, 1H), 3.72- |
| | | 3.64 (m, 1H), 3.61 (s, 3H), 3.35 |
| | | (s, 3H), 2.76-2.67 (m, |
| | | 1H), 2.59-2.52 (m, 1H), 2.38-2.28 |
| | | (m, 1H), 2.12-2.06 |
| | | (m, 1H), 1.56 (s, 6H), |
| | | 1.30 (d, J = 6.8 Hz, 6H). |
| A76 | mass | ¹H NMR (400 MHz, DMSO) |
| | calcd. for | δ 10.88 (s, 1H), 9.45 (d, J = |
| | C₄₄H₃₇N₉O₅, | 1.5 Hz, 1H), 9.31 (t, J = 5.9 Hz, 1H), |
| | 723.8; m/z | 8.99 (s, 1H), 8.74 |
| | found, 724.4 | (dd, J = 8.2, 2.1 Hz, 1H), |
| | [M + H]⁺. | 8.17 (d, J = 8.3 Hz, 1H), 7.96 (s, |
| | | 1H), 7.72 (d, J = 1.1 Hz, 1H), |
| | | 7.58 (d, J = 8.6 Hz, 1H), |
| | | 7.37 (dd, J = 8.6, 1.5 Hz, 1H), |
| | | 7.00 (s, 1H), 4.37 (d, J = |
| | | 5.9 Hz, 2H), 4.16 (dd, J = 12.2, |
| | | 4.9 Hz, 1H), 3.91-3.84 |
| | | (m, 2H), 3.61-3.55 (m, 1H), 3.54 |
| | | (s, 3H), 3.34 (s, 3H), |
| | | 3.02 (t, J = 6.4 Hz, 2H), 2.76-2.67 |
| | | (m, 1H), 2.60-2.53 |
| | | (m, 1H), 2.39-2.28 (m, 1H), |
| | | 2.14-2.05 (m, 3H), 1.25 |
| | | (d, J = 6.7 Hz, 6H). |
| A77 | mass | ¹H NMR (400 MHz, DMSO-d₆) |
| | calcd. for | δ 10.88 (s, 1H), 9.38- |
| | C₃₉H₃₄N₈O₆, | 9.28 (m, 2H), 8.64 (dd, J = 8.2, |
| | 710.3; m/z | 1.8 Hz, 1H), 8.14 (d, J = |
| | found, 711.8 | 8.2 Hz, 1H), 8.05 (s, 1H), 7.96 |
| | [M + H]⁺. | (s, 1H), 7.71 (s, 1H), 7.57 |
| | | (d, J = 8.5 Hz, 1H), 7.36 (d, J = 8.6 Hz, |
| | | 1H), 7.17 (s, 1H), |
| | | 6.88 (s, 1H), 4.65-4.58 (m, 2H), |
| | | 4.36 (d, J = 5.8 Hz, 2H), |
| | | 4.18-4.12 (m, 1H), 3.89-3.83 |
| | | (m, 2H), 3.80 (s, 2H), |
| | | 3.58 (s, 3H), 2.76-2.67 (m, 1H), |
| | | 2.60 (s, 3H), 2.58-2.52 |
| | | (m, 1H), 2.38-2.29 (m, 1H), |
| | | 2.12-2.05 (m, 1H), 1.21- |
| | | 1.15 (m, 3H). |
| A78 | mass | ¹H NMR (400 MHz, DMSO-d₆) |
| | calcd. for | δ 10.89 (s, 1H), 9.44 (d, J = |
| | C₄₂H₃₉N₉O₆, | 1.6 Hz, 1H), 9.31 (t, J = 5.9 Hz, 1H), |
| | 765.3; m/z | 8.95 (s, 1H), 8.77- |
| | found, 766.3 | 8.70 (m, 1H), 8.16 (d, J = 8.2 Hz, 1H), |
| | [M + H]⁺. | 7.96 (s, 1H), 7.75- |
| | | 7.69 (m, 1H), 7.57 (d, J = 8.5 Hz, |
| | | 1H), 7.40-7.32 (m, |
| | | 1H), 7.01 (s, 1H), 4.38 (d, J = 5.8 Hz, |
| | | 2H), 4.16 (dd, J = |
| | | 12.2, 4.8 Hz, 1H), 3.96-3.90 |
| | | (m, 2H), 3.90-3.84 (m, |
| | | 2H), 3.57 (s, 3H), 3.54-3.48 |
| | | (m, 2H), 3.47-3.43 (m, |
| | | 1H), 3.34 (s, 3H), 3.07-2.99 |
| | | (m, 2H), 2.77-2.67 (m, |
| | | 1H), 2.60-2.54 (m, 1H), 2.40-2.30 |
| | | (m, 1H), 2.14-2.06 |
| | | (m, 3H), 1.95-1.85 (m, 2H), |
| | | 1.73-1.65 (m, 2H). |
| A79 | mass | ¹H NMR (400 MHz, DMSO-d₆) |
| | calcd. for | δ 10.89 (s, 1H), 9.18 (d, J = |
| | C₃₉H₃₄N₈O₅, | 1.6 Hz, 1H), 9.12 (t, J = 6.0 Hz, 1H), |
| | 694.27; m/z | 8.40 (d, J = 1.2 |
| | found, 695.4 | Hz, 1H), 7.96 (s, 1H), 7.87 |
| | [M + H]⁺. | (s, 1H), 7.71 (d, J = 0.8 Hz, |
| | | 1H), 7.58 (d, J = 8.6 Hz, 1H), |
| | | 7.37 (dd, J = 8.8, 1.2 Hz, |
| | | 1H), 7.24 (d, J = 8.0 Hz, 2H), |
| | | 7.08 (dd, J = 8.4, 1.6 Hz, |
| | | 1H), 4.33 (d, J = 6.0 Hz, 2H), |
| | | 4.15 (dd, J = 12.4, 4.8 Hz, |
| | | 1H), 3.70-3.65 (m, 2H), 3.36 (s, 3H), |
| | | 3.33 (s, 3H), 3.04 |
| | | (t, J = 6.4 Hz, 2H), 2.77-2.67 (m, |
| | | 1H), 2.64 (s, 3H), 2.60- |
| | | 2.54 (m, 1H), 2.37-2.29 (m, 1H), |
| | | 2.18-2.13 (m, 2H), |
| | | 2.11-2.05 (m, 1H). |
| A80 | mass | ¹H NMR (400 MHz, DMSO-d₆) |
| | calcd. for | δ 10.89 (s, 1H), 9.24 (s, |
| | C₄₀H₃₆N₈O₇, | 1H), 9.15 (t, J = 5.9 Hz, 1H), |
| | 740.2; m/z | 8.48-8.42 (m, 3H), 8.00- |
| | found, 741.3 | 7.93 (m, 1H), 7.74-7.70 |
| | [M + H]⁺. | (m, 1H), 7.58 (d, J = 8.5 Hz, |
| | | 1H), 7.39-7.35 (m, 1H), 6.89 |
| | | (d, J = 1.7 Hz, 1H), 6.66- |
| | | 6.61 (m, 1H), 4.36-4.30 (m, 2H), |
| | | 4.18-4.13 (m, 1H), |
| | | 3.78-3.72 (m, 4H), 3.39 |
| | | (s, 3H), 3.30-3.30 (m, 3H), |
| | | 3.22-3.21 (m, 4H), 2.69-2.63 |
| | | (m, 4H), 2.55-2.54 (m, |
| | | 1H), 2.33-2.32 (m, 1H), 2.09-2.06 (m, 1H). |
| A81 | mass | ¹H NMR (400 MHz, DMSO-d₆) |
| | calcd. for | δ 10.90-10.84 (m, 1H), |
| | C₄₀H₃₄F₂N₆O₅, | 9.39-9.33 (m, 2H), 8.97 (d, J = 1.6 |
| | 716.3; | Hz, 1H), 8.69 (dd, J = |
| | m/z found, | 8.2, 2.2 Hz, 1H), 8.29 |
| | 717.2 | (d, J = 8.3 Hz, 1H), 8.20 (d, J = |
| | [M + H]⁺. | 8.3 Hz, 1H), 8.16 (dd, J = 8.4, 2.2 Hz, |
| | | 1H), 7.96 (s, 1H), |
| | | 7.71 (d, J = 1.2 Hz, 1H), 7.57 |
| | | (d, J = 8.5 Hz, 1H), 7.36 |
| | | (dd, J = 8.5, 1.6 Hz, 1H), 7.27-7.21 |
| | | (m, 2H), 4.37 (d, J = |
| | | 5.9 Hz, 2H), 4.15 (dd, J = 12.2, 4.8 |
| | | Hz, 1H), 3.71-3.63 |
| | | (m, 1H), 3.59 (s, 3H), 3.35 |
| | | (s, 3H), 2.76-2.67 (m, 1H), |
| | | 2.59-2.53 (m, 1H), 2.38-2.28 |
| | | (m, 1H), 2.12-2.05 (m, |
| | | 1H), 1.27 (d, J = 6.8 Hz, 6H). |
| A82 | mass | ¹H NMR (400 MHz, DMSO-d₆) |
| | calcd. for | δ 10.90 (s, 1H), 9.23 (s, |
| | C₄₂H₃₇D₃N₈O₆, | 1H), 9.15 (s, 1H), 8.45 (s, 1H), 8.38 |
| | 755.3; | (d, J = 2.7 Hz, 2H), |
| | m/z found, | 7.97 (d, J = 2.2 Hz, 1H), 7.72 |
| | 756.3 | (s, 1H), 7.62-7.56 (m, 1H), |
| | [M + H]⁺. | 7.40-7.34 (m, 1H), 7.09 (s, 1H), |
| | | 6.99 (s, 1H), 4.37-4.30 |
| | | (m, 2H), 4.19-4.13 (m, 1H), |
| | | 3.97-3.91 (m, 2H), 3.64- |
| | | 3.61 (m, 3H), 3.55-3.49 |
| | | (m, 3H), 3.33-3.31 (m, 3H), |
| | | 2.77-2.69 (m, 1H), 2.67-2.63 |
| | | (m, 3H), 2.60-2.55 (m, |
| | | 1H), 2.39-2.30 (m, 1H), 2.13-2.06 |
| | | (m, 1H), 1.80-1.71 |
| | | (m, 4H). |
| A83 | mass | ¹H NMR (400 MHz, DMSO-d₆) |
| | calcd. for | δ 10.88 (s, 1H), 9.34- |
| | C₄₄H₄₂N₆O₆, | 9.23 (m, 2H), 8.62-8.54 (m, 1H), |
| | 750.3; m/z | 8.16-8.06 (m, 1H), |
| | found, 751.8 | 7.95 (s, 1H), 7.86 (s, 1H), |
| | [M + H]⁺. | 7.71 (d, J = 1.2 Hz, 1H), 7.61 |
| | | (s, 1H), 7.58-7.55 (m, 1H), 7.39-7.33 |
| | | (m, 1H), 7.06 (s, |
| | | 1H), 6.89 (d, J = 1.2 Hz, 1H), |
| | | 4.38-4.26 (m, 3H), 4.15 |
| | | (dd, J = 12.2, 4.8 Hz, 1H), |
| | | 3.69-3.62 (m, 1H), 3.58 (s, |
| | | 3H), 3.26 (s, 3H), 2.76-2.67 |
| | | (m, 1H), 2.59-2.53 (m, |
| | | 1H), 2.38-2.28 (m, 1H), 2.20-2.12 |
| | | (m, 2H), 2.11-2.04 |
| | | (m, 1H), 1.51 (s, 3H), 1.41 |
| | | (s, 3H), 1.33-1.30 (m, 3H), |
| | | 1.28-1.25 (m, 3H). |
| A84 | mass | ¹H NMR (400 MHz, DMSO-d₆) |
| | calcd. for | δ 10.89 (s, 1H), 9.48 (s, |
| | C₄₁H₃₂N₆O₅, | 1H), 9.38-9.33 (m, 1H), 9.32-9.31 |
| | 688.2; m/z | (m, 1H), 8.80-8.75 |
| | found, 689.2 | (m, 1H), 8.64 (s, 1H), 8.24-8.20 |
| | [M + H]⁺. | (m, 1H), 7.96 (s, 1H), |
| | | 7.87 (s, 1H), 7.72 (s, 1H), |
| | | 7.60-7.57 (m, 1H), 7.53 (s, |
| | | 1H), 7.43 (s, 1H), 7.39-7.36 |
| | | (m, 1H), 7.36-7.33 (m, |
| | | 1H), 7.31-7.27 (m, 1H), |
| | | 4.39 (s, 2H), 4.18-4.13 (m, |
| | | 1H), 3.44-3.42 (m, 3H), 3.41-3.40 |
| | | (m, 3H), 2.73-2.69 |
| | | (m, 1H), 2.60 (s, 3H), 2.53 (s, 1H), |
| | | 2.38-2.31 (m, 1H), |
| | | 2.09 (s, 1H). |
| A85 | mass | LC-MS (ESI): mass calcd. |
| | calcd. for | for C₄₅H₄₂FN₅O₆, 767.31; m/z |
| | C₄₅H₄₂FN₅O₆, | found, 768.31 [M + H]⁺. |
| | 767.31; | ¹HNMR(400 MHz, DMSO-d₆) |
| | m/z found, | δ 10.88 (s, 1H), 9.30 (t, J = |
| | 768.31 | 5.9 Hz, 1H), 8.81 (s, 1H), 8.14 (dd, |
| | [M + H]⁺. | J = 19.9, 8.1 Hz, 2H), |
| | | 7.95 (s, 1H), 7.71 (d,J = 1.2Hz, |
| | | 1H), 7.57 (d, J = 8.5 Hz, |
| | | 1H), 7.36 (dd, J = 8.5, 1.5 Hz, 1H), |
| | | 7.09 (d, J = 7.0 Hz, |
| | | 1H), 7.02 (d, J = 0.6 Hz, 1H), |
| | | 6.86 (d, J = 1.0 Hz, 1H), |
| | | 6.49 (d, J = 11.7 Hz, 1H), 4.35 |
| | | (d, J = 5.7 Hz, 2H), 4.24 |
| | | (dd, J = 11.5, 7.6 Hz, 1H), 4.15 (dd, |
| | | J = 12.2, 4.7 Hz, 1H), |
| | | 3.65-3.60 (m, 1H), 3.57 (s,3H), |
| | | 3.27 (s, 3H), 2.72-2.66 |
| | | (m, 1H), 2.58-2.53 (m, 1H), |
| | | 2.37-2.30 (m, 1H), 2.13- |
| | | 2.06 (m, 3H), 1.46 (s, 3H), |
| | | 1.36 (s, 3H), 1.32-1.30 (d, J = |
| | | 6.8 Hz, 3H), 1.26 |
| | | (d, J = 6.8 Hz, 3H). |
| A86 | mass | ¹H NMR (400 MHz, DMSO-d₆) |
| | calcd. for | δ 10.88 (s, 1H), 9.87 (s, |
| | C₄₅H₃₈N₈O₆, | 1H), 9.49 (d, J = 1.7 Hz, 1H), 9.34 |
| | 786.3; m/z | (s, 1H), 8.81 (dd, J = |
| | found, 787.3 | 8.2, 2.2 Hz, 1H), 8.75 (s, 1H), |
| | [M + H]⁺. | 8.23 (d, J = 8.2 Hz, 1H), |
| | | 8.13 (d, J = 7.9 Hz, 1H), 7.97-7.87 |
| | | (m, 3H), 7.73 (d, J = |
| | | 1.2 Hz, 1H), 7.58 (d, J = 9.0 Hz, |
| | | 2H), 7.38 (dd, J = 8.6, |
| | | 1.6 Hz, 1H), 4.42-4.37 |
| | | (m, 4H), 4.19-4.12 (m, 1H), |
| | | 3.70 (s, 3H), 3.42 (s, 3H), 3.26-3.22 |
| | | (m, 2H), 3.08-3.03 |
| | | (m, 2H), 2.76-2.68 (m, 1H), |
| | | 2.60-2.54 (m, 1H), 2.39- |
| | | 2.30 (m, 1H), 2.15-2.07 (m, 3H), |
| | | 1.93-1.86 (m, 2H). |
| A87 | mass | ¹H NMR (400 MHz, DMSO-d₆) |
| | calcd. for | δ 10.89 (s, 1H), 9.40 (s, |
| | C₄₂H₃₄N₆O₅, | 1H), 9.32-9.29 (m, 1H), 9.18 (t, |
| | 702.2; m/z | J = 5.9 Hz, 1H), 8.73 (s, |
| | found, 703.3 | 1H), 8.60-8.54 (m, 1H), |
| | [M + H]⁺. | 8.11-8.04 (m, 1H), 7.97 (s, |
| | | 1H), 7.90 (t, J = 7.7 Hz, 1H), |
| | | 7.76-7.70 (m, 1H), 7.69- |
| | | 7.63 (m, 1H), 7.62-7.56 (m, 1H), |
| | | 7.43-7.35 (m, 1H), |
| | | 7.27 (s, 1H), 7.07 (s, 1H), |
| | | 4.35 (d, J = 5.8 Hz, 2H), 4.16 |
| | | (dd, J = 12.2, 4.9 Hz, 1H), 3.65 |
| | | (s, 3H), 3.37 (s, 3H), 2.78- |
| | | 2.71 (m, 1H), 2.71-2.68 (m, 6H), |
| | | 2.60-2.53 (m, 1H), |
| | | 2.39-2.30 (m, 1H), |
| | | 2.13-2.04 (m, 1H). |
| A88 | mass | ¹H NMR (400 MHz, DMSO-d₆) |
| | calcd. for | δ 10.89 (s, 1H), 9.24- |
| | C₃₉H₃₄N₈O₆, | 9.04 (m, 2H), 8.39 (s, 1H), 8.04 |
| | 710.26; m/z | (s, 1H), 7.96 (s, 1H), 7.71 |
| | found, 711 | (s, 1H), 7.58 (d, J = 8.6 Hz, 1H), |
| | [M + H]⁺. | 7.37 (d, J = 7.2 Hz, 1H), |
| | | 7.11 (s, 1H), 6.89 (s, 1H), 4.65-4.57 |
| | | (m, 2H), 4.32 (d, J = |
| | | 5.8 Hz, 2H), 4.19-4.11 (m, 1H), |
| | | 3.82-3.76 (m, 2H), |
| | | 3.59 (s, 3H), 3.31 (s, 3H), |
| | | 2.73-2.66 (m, 1H), 2.64 (s, |
| | | 3H), 2.60 (s, 3H), 2.57-2.53 |
| | | (m, 1H), 2.37-2.29 (m, |
| | | 1H), 2.13-2.04 (m, 1H). |
| A89 | mass | ¹H NMR (400 MHz, DMSO-d₆) |
| | calcd. for | δ 10.88 (s, 1H), 9.36 (d, J = |
| | C₄₀H₃₆N₈O₆, | 2.0 Hz, 1H), 9.31 (t, J = 5.9 Hz, |
| | 724.4; m/z | 1H), 8.65 (dd, J = 8.2, |
| | found, 725.4 | 2.1 Hz, 1H), 8.14 (d, J = 8.3 Hz, 1H), |
| | [M + H]⁺. | 8.03 (s, 1H), 7.96 (s, |
| | | 1H), 7.71 (s, 1H), 7.57 (d, J = 8.5 |
| | | Hz, 1H), 7.36 (dd, J = |
| | | 8.5, 1.6 Hz, 1H), 7.12 (d, J = 2.0 Hz, |
| | | 1H), 7.05 (d, J = 1.9 |
| | | Hz, 1H), 4.63 (s, 2H), 4.35 |
| | | (d, J = 5.9 Hz, 2H), 4.15 (dd, J = |
| | | 12.3, 4.8 Hz, 1H), 3.83 (s, 2H), |
| | | 3.70-3.63 (m, 1H), |
| | | 3.59 (s, 3H), 3.32 (s, 3H), 2.73-2.67 |
| | | (m, 1H), 2.61-2.53 |
| | | (m, 1H), 2.35-2.30 (m, 1H), |
| | | 2.10-2.06 (m, 1H), 1.29 |
| | | (d, J = 6.8 Hz, 6H). |
| A90 | mass | ¹H NMR (400 MHz, DMSO-d₆) |
| | calcd. for | δ 10.89 (s, 1H), 9.45- |
| | C₄₁H₃₉N₉O₆, | 9.32 (m, 1H), 9.12-8.97 |
| | 753.3; m/z | (m, 1H), 8.96-8.82 (m, 1H), |
| | found, 754.3 | 8.38-8.24 (m, 1H), 7.97 (s, 1H), |
| | [M + H]⁺. | 7.72 (s, 1H), 7.59 (d, J = |
| | | 8.4 Hz, 1H), 7.41-7.34 (m, 1H), |
| | | 7.24-7.02 (m, 2H), |
| | | 4.34 (s, 2H), 4.16 (d, J = 9.3 Hz, 1H), |
| | | 3.94 (d, J = 10.7 |
| | | Hz, 2H), 3.66-3.63 (m, 3H), |
| | | 3.62-3.57 (m, 3H), 3.54- |
| | | 3.50 (m, 3H), 3.33-3.32 (m, 3H), |
| | | 3.18-3.07 (m, 1H), |
| | | 2.72-2.66 (m, 2H), 2.63-2.57 |
| | | (m, 2H), 2.38-2.31 (m, |
| | | 1H), 2.13-2.06 (m, 1H), |
| | | 1.83-1.72 (m, 4H). |
| A91 | mass | ¹H NMR (400 MHz, DMSO-d₆) |
| | calcd. for | δ 10.90 (s, 1H), 9.22 (s, |
| | C₄₁H₃₈N₁₀O₇, | 1H), 9.20-9.15 (m, 2H), 8.47 |
| | 782.3; m/z | (s, 1H), 7.97 (s, 1H), 7.73 |
| | found, 783.7 | (s, 1H), 7.59 (d, J = 8.5 Hz, 1H), |
| | [M + H]⁺. | 7.38 (d, J = 8.5 Hz, 1H), |
| | | 6.92 (s, 1H), 4.59-4.56 (m, 2H), |
| | | 4.34 (d, J = 5.7 Hz, 2H), |
| | | 4.17 (dd, J = 12.3, 4.8 Hz, 1H), |
| | | 4.03-3.98 (m, 2H), 3.81- |
| | | 3.76 (m, 4H), 3.53 (s, 3H), |
| | | 3.34 (s, 3H), 3.10-3.04 (m, |
| | | 4H), 2.76-2.68 (m, 1H), 2.66 |
| | | (s, 3H), 2.60-2.55 (m, |
| | | 1H), 2.39-2.30 (m, 1H), |
| | | 2.13-2.06 (m, 1H). |
| A92 | mass | ¹H NMR (400 MHz, DMSO-d₆) |
| | calcd. for | δ 10.94-10.82 (m, 1H), |
| | C₃₈H₃₂N₈O₆, | 9.31-9.01 (m, 2H), 8.39 (s, 1H), |
| | 696.0; m/z | 7.98 (d, J = 13.5 Hz, |
| | found, 697.0 | 2H), 7.72 (s, 1H), 7.58 (d, J = 8.5 Hz, |
| | [M + H]⁺. | 1H), 7.37 (d, J = 8.6 |
| | | Hz, 1H), 7.29 (s, 1H), 7.26-7.22 |
| | | (m, 1H), 7.14-7.09 (m, |
| | | 1H), 4.62 (s, 2H), 4.33 (d, J = 5.7 |
| | | Hz, 2H), 4.16 (dd, J = |
| | | 12.3, 4.7 Hz, 1H), 3.81 (s, 2H), |
| | | 3.38 (s, 3H), 3.33-3.31 |
| | | (m, 3H), 2.75-2.68 (m, 1H), 2.64 |
| | | (s, 3H), 2.60-2.55 (m, |
| | | 1H), 2.37-2.28 (m, 1H), |
| | | 2.13-2.05 (m, 1H). |
| A93 | mass | ¹H NMR (400 MHz, DMSO-d₆) |
| | calcd. for | δ 10.88 (s, 1H), 9.74 (d, J = |
| | C₃₉H₂₉N₇O₅, | 1.8 Hz, 1H), 9.64 (s, 1H), 9.42 |
| | 675.22; m/z | (t, J = 6.0 Hz, 1H), 9.06 |
| | found, 676.22 | (dd, J = 8.2, 2.1 Hz, 2H), |
| | [M + H]⁺. | 8.25 (t, J = 27.8 Hz, 1H), 8.14 (t, |
| | | J = 6.5 Hz, 2H), 7.96 (s, 1H), |
| | | 7.83-7.75 (m, 1H), 7.73 (d, |
| | | J = 1.2 Hz, 1H), 7.58 (d, |
| | | J = 8.6 Hz, 1H), 7.50 (s, 1H), |
| | | 7.41-7.31 (m, 2H), 4.39 |
| | | (d, J = 5.9 Hz, 2H), 4.16-4.10 |
| | | (m, 1H), 3.43 (s, 3H), 3.41 (s, 3H), |
| | | 2.72-2.60 (m, 1H), |
| | | 2.58-2.55 (m, 1H), 2.34-2.30 |
| | | (m, 1H), 2.15-2.01 (m, |
| | | 1H) |
| A94 | mass | ¹H NMR (400 MHz, DMSO-d₆) |
| | calcd. for | δ 10.88 (s, 1H), 9.15- |
| | C₄₂H₃₆F₂N₆O₅, | 9.09 (m, 1H), 8.38-8.35 (m, 1H), |
| | 742.3; | 7.96 (s, 1H), 7.73- |
| | m/z found, | 7.66 (m, 2H), 7.59-7.56 |
| | 743.3 | (m, 1H), 7.38-7.34 (m, 1H), |
| | [M + H]⁺. | 7.25-7.22 (m, 1H), 7.18 (s, 1H), |
| | | 7.09 (s, 1H), 7.03-6.99 |
| | | (m, 1H), 6.81-6.54 (m, 2H), |
| | | 4.40-4.27 (m, 2H), 4.18- |
| | | 4.13 (m, 1H), 3.65-3.61 (m, 2H), |
| | | 3.36 (s, 3H), 3.33 (s, |
| | | 3H), 2.93-2.89 (m, 2H), |
| | | 2.70-2.66 (m, 1H), 2.62 (s, |
| | | 3H), 2.59-2.54 (m, 1H), 2.34-2.31 |
| | | (m, 1H), 2.10-2.02 |
| | | (m, 3H). |
| A95 | mass | ¹H NMR (400 MHz, DMSO-d₆) |
| | calcd. for | δ 10.88 (s, 1H), 9.22 (d, J = |
| | C₄₃H₄₂N₈O₆, | 1.8 Hz, 1H), 9.14 (t, J = 5.9 Hz, 1H), |
| | 766.3; m/z | 8.43 (d, J = 1.5 |
| | found, 767.3 | Hz, 1H), 8.31 (s, 2H), 7.96 (s, 1H), |
| | [M + H]⁺. | 7.72 (s, 1H), 7.58 (d, J = |
| | | 8.5 Hz, 1H), 7.37 (dd, J = 8.6, 1.5 |
| | | Hz, 1H), 7.05 (d, J = |
| | | 1.8 Hz, 1H), 6.94 (d, J = 1.8 Hz, 1H), |
| | | 4.33 (d, J = 5.8 Hz, |
| | | 2H), 4.16 (dd, J = 12.2, 4.9 Hz, |
| | | 1H), 3.97-3.90 (m, 2H), |
| | | 3.89-3.80 (m, 2H), 3.62 (s, 3H), |
| | | 3.56-3.48 (m, 3H), |
| | | 3.32 (s, 3H), 2.77-2.68 (m, 1H), |
| | | 2.64 (s, 3H), 2.60-2.54 |
| | | (m, 1H), 2.39-2.29 (m, 1H), |
| | | 2.13-2.04 (m, 1H), 1.78- |
| | | 1.70 (m, 4H), 1.20 (t, |
| | | J = 7.0 Hz, 3H). |
| A96 | mass | ¹H NMR (400 MHz, DMSO-d₆) |
| | calcd. for | δ 10.89 (s, 1H), 9.52- |
| | C₄₁H₃₂N₆O₅, | 9.47 (m, 1H), 9.41 (s, 1H), 9.34 (t, |
| | 688.3; m/z | J = 5.9 Hz, 1H), 8.81- |
| | found, 689.7 | 8.76 (m, 1H), 8.74 (s, 1H), |
| | [M + H]⁺. | 8.23-8.20 (m, 1H), 8.10- |
| | | 8.05 (m, 1H), 7.96 (s, 1H), |
| | | 7.92-7.87 (m, 1H), 7.75- |
| | | 7.71 (m, 1H), 7.67-7.63 (m, 1H), |
| | | 7.59-7.56 (m, 1H), |
| | | 7.40-7.35 (m, 1H), 7.26 |
| | | (d, J = 1.1 Hz, 1H), 7.05 (s, 1H), |
| | | 4.39 (d, J = 5.9 Hz, 2H), 4.19-4.13 |
| | | (m, 1H), 3.64 (s, 3H), |
| | | 3.42-3.37 (m, 3H), 2.78-2.69 |
| | | (m, 1H), 2.68 (s, 3H), |
| | | 2.61-2.54 (m, 1H), 2.39-2.29 |
| | | (m, 1H), 2.12-2.05 (m, |
| | | 1H) |
| A97 | mass | ¹H NMR (400 MHz, DMSO-d₆) |
| | calcd. for | δ 10.88 (s, 1H), 9.44 (d, J = |
| | C₄₃H₃₇N₇O₆, | 1.7 Hz, 1H), 9.36-9.30 |
| | 747.28; m/z | (m, 1H), 9.11 (s, 1H), 8.80- |
| | found, 748 | 8.74 (m, 2H), 8.20 (d, J = 8.3 Hz, |
| | [M + H]⁺. | 1H), 7.96 (s, 1H), 7.72 |
| | | (s, 2H), 7.58 (d, J = 8.5 Hz, 1H), |
| | | 7.40-7.35 (m, 1H), 7.23- |
| | | 7.16 (m, 2H), 4.41-4.35 (m, 2H), |
| | | 4.15 (dd, J = 12.4, |
| | | 5.0 Hz, 1H), 3.76-3.69 (m, 1H), |
| | | 3.67-3.62 (m, 6H), |
| | | 3.37 (s, 3H), 2.72-2.66 (m, 1H), |
| | | 2.60-2.56 (m, 1H), |
| | | 2.36-2.30 (m, 1H), 2.11-2.06 (m, |
| | | 1H), 1.36-1.31 (m, |
| | | 6H). |
| A98 | mass | ¹H NMR (400 MHz, DMSO-d₆) |
| | calcd. for | δ 10.89 (s, 1H), 9.20 (t, J = |
| | C₄₂H₃₈N₈O₅, | 5.8 Hz, 1H), 8.87 (d, J = 1.8 |
| | 734.30; m/z | Hz, 1H), 8.32 (d, J = 2.9 |
| | found, 735.30 | Hz, 1H), 8.13 (d, J = 1.4 Hz, 1H), |
| | [M + H]⁺. | 7.97 (s, 1H), 7.73 (s, |
| | | 1H), 7.59 (d, J = 1.5 Hz, 1H), |
| | | 7.58 (d, J = 3.8 Hz, 1H), |
| | | 7.40-7.36 (m, 1H), 7.05 (d, J = 1.8 |
| | | Hz, 1H), 6.97 (d, J = |
| | | 1.7 Hz, 1H), 4.34 (d, |
| | | J = 5.8 Hz, 2H), 4.19-4.14 (m, 1H), |
| | | 3.69-3.64 (m, 1H),3.60 (s, 3H), |
| | | 3.40 (s, 3H), 3.32 (s, |
| | | 3H), 2.76-2.69 (m, 1H), 2.64 |
| | | (s, 3H), 2.60-2.55 (m, |
| | | 1H), 2.38-2.29 (m, 1H), 2.13-2.07 |
| | | (m, 1H), 1.28 (d, J = |
| | | 6.8 Hz, 6H). |
| A99 | mass | ¹H NMR (400 MHz, DMSO-d₆) |
| | calcd. for | δ 10.88 (s, 1H), 9.79 (s, |
| | C₄₄H₃₆N₈O₆, | 1H), 9.48 (s, 1H), 9.33 (d, J = 5.6 Hz, |
| | 772; m/z | 1H), 8.86-8.71 (m, |
| | found, 773 | 2H), 8.23 (d, J = 7.9 Hz, 1H), |
| | [M + H]⁺. | 8.13 (d, J = 8.1 Hz, 1H), |
| | | 7.97-7.90 (m, 2H), 7.87 (d, J = 6.9 Hz, |
| | | 1H), 7.73 (s, 1H), |
| | | 7.58 (d, J = 8.5 Hz, 1H), 7.38 |
| | | (d, J = 9.5 Hz, 2H), 4.49 (d, |
| | | J = 6.0 Hz, 2H), 4.39 (d, |
| | | J = 5.9 Hz, 2H), 4.30 (d, J = 10.2 |
| | | Hz, 2H), 4.16 (dd, J = 12.1, 5.2 Hz, |
| | | 1H), 3.92 (d, J = 10.2 |
| | | Hz, 2H), 3.54 (s, 3H), 3.41 (s, 3H), |
| | | 2.95-2.91 (m, 1H), |
| | | 2.69-2.63 (m, 1H), 2.60-2.54 |
| | | (m, 1H), 2.39-2.32 (m, |
| | | 1H), 2.17-2.10 (m, 1H), |
| | | 1.97 (d, J = 8.1 Hz, 1H). |
| A100 | mass | ¹H NMR (400 MHz, DMSO-d₆) |
| | calcd. for | δ 10.88 (s, 1H), 9.20 (dd, |
| | C₄₂H₃₉N₇O₅, | J = 12.2, 3.9 Hz, 2H), 8.46 (dd, |
| | 721.30; m/z | J = 8.3, 2.2 Hz, 1H), 8.07 |
| | found, 722.30 | (d, J = 8.2 Hz, 1H), 7.95 (s, |
| | [M + H]⁺. | 1H), 7.83 (s, 1H), 7.74 (s, |
| | | 1H), 7.71 (d, J = 1.2 Hz, 1H), |
| | | 7.57 (d, J = 8.5 Hz, 1H), |
| | | 7.36 (dd, J = 8.6, 1.6 Hz, 1H), |
| | | 7.03 (d, J = 1.9 Hz, 1H), |
| | | 6.97 (d, J = 1.9 Hz, 1H), |
| | | 4.35 (d, J = 5.9 Hz, 2H), 4.15 |
| | | (dd, J = 12.3, 4.8 Hz, 1H), |
| | | 3.66-3.60 (m, 3H), 3.60 (s, |
| | | 3H), 3.31 (s, 2H), 2.93-2.88 |
| | | (m, 2H), 2.72-2.67 (m, |
| | | 1H), 2.59-2.55 (m, 1H), 2.35-2.31 |
| | | (m, 1H), 2.09-2.05 |
| | | (m, 3H), 2.02-1.98 (m, 1H), 1.28 |
| | | (d, J = 6.8 Hz, 6H). |
| A101 | mass | ¹HNMR (400 MHz, DMSO-d₆) |
| | calcd. for | δ 10.88 (s, 1H), 9.50 (d, J = |
| | C₃₉H₂₇N₅O₆, | 1.7 Hz, 1H), 9.39-9.28 |
| | 661.67; m/z | (m, 2H), 8.83-8.72 (m, 2H), |
| | found, 662.6 | 8.23 (d, J = 8.4 Hz, 1H), |
| | [M + H]⁺. | 8.14 (d, J = 8.4 Hz, 1H), 7.98- |
| | | 7.90 (m, 2H), 7.74-7.66 (m, 2H), |
| | | 7.60-7.56 (m, 2H), |
| | | 7.54 (d, J = 8.2 Hz, 1H), 7.36 |
| | | (t, 2H), 4.38 (d, J = 5.9 Hz, |
| | | 2H), 4.15 (dd, J = 12.4, 4.7 Hz, |
| | | 1H), 3.41 (s, 3H), 2.72- |
| | | 2.67 (m, 1H), 2.59-2.56 (m, 1H), |
| | | 2.34-2.31 (m, 1H), |
| | | 2.11-2.06 (m, 1H). |
| A102 | mass | ¹HNMR (400 MHz, DMSO-d₆) |
| | calcd. for | δ 10.88 (s, 1H), 9.87 (s, |
| | C₄₄H₃₉N₉O₅, | 1H), 9.49 (s, 1H), 9.33 (t, J = 5.8 |
| | 773.31; m/z | Hz, 1H), 8.79 (d, J = |
| | found, 774.31 | 13.6 Hz, 2H), 8.25 (d, J = 8.1 |
| | [M + H]⁺. | Hz, 1H), 8.15 (d, J = 8.1 |
| | | Hz,1H), 8.02-7.86 (m, 3H), |
| | | 7.72 (s, 1H), 7.64 (s, 1H), |
| | | 7.59 (d, J = 8.5 Hz, 1H), 7.37 |
| | | (d, J = 8.5 Hz, 1H), 4.39 (d, |
| | | J = 5.9 Hz, 2H), 4.16 (dd, |
| | | J = 12.2, 4.8 Hz, 1H), 3.65 (s, |
| | | 3H), 3.52 (s, 3H), 3.44-3.34 |
| | | (m, 3H), 3.32-3.28 (m, |
| | | 2H), 3.26-3.24 (m, 2H), 2.88 |
| | | (s, 3H), 2.77-2.66 (m, |
| | | 1H), 2.57-2.50 (m, 2H), 2.33-2.22 |
| | | (m, 1H), 2.09-1.98 |
| | | (m, 1H). |
| A103 | mass | ¹H NMR (400 MHz, DMSO-d₆) |
| | calcd. for | δ 10.89 (s, 1H), 9.23 (s, |
| | C₃₇H₃₂N₈O₅, | 1H), 9.17-9.13 (m, 1H), 8.44 |
| | 668.2; m/z | (s, 1H), 8.39-8.35 (m, |
| | found, 669.4 | 2H), 7.98-7.95 (m, 1H), |
| | [M + H]⁺. | 7.72 (s, 1H), 7.60-7.56 (m, |
| | | 1H), 7.39-7.36 (m, 1H), 7.25-7.21 |
| | | (m, 2H), 7.07-7.04 |
| | | (m, 1H), 4.36-4.30 (m, 2H), |
| | | 4.19-4.14 (m, 1H), 3.40 (s, |
| | | 3H), 3.33-3.32 (m, 6H), |
| | | 2.74-2.71 (m, 1H), 2.65 (s, |
| | | 3H), 2.60-2.55 (m, 2H), |
| | | 2.35-2.33 (m, 1H). |
| A104 | mass | ¹H NMR (400 MHz, DMSO-d₆) |
| | calcd. for | δ 10.88 (s, 1H), 9.52- |
| | C₄₀H₂₉N₅O₅, | 9.47 (m, 1H), 9.38-9.32 |
| | 659.2; m/z | (m, 2H), 8.81-8.76 (m, 2H), |
| | found, 660.3 | 8.24-8.21 (m, 1H), |
| | [M + H]⁺. | 8.11-8.08 (m, 1H), 7.96 (s, 1H), |
| | | 7.93-7.89 (m, 1H), 7.73-7.71 |
| | | (m, 1H), 7.65-7.63 (m, |
| | | 1H), 7.59-7.57 (m, 1H), 7.54-7.51 |
| | | (m, 2H), 7.39-7.36 |
| | | (m, 1H), 7.21-7.18 (m, 1H), |
| | | 4.38 (d, J = 5.8 Hz, 2H), |
| | | 4.18-4.13 (m, 1H), 3.70 (s, 2H), |
| | | 3.22 (s, 3H), 2.73-2.66 |
| | | (m, 1H), 2.60-2.53 (m, 1H), |
| | | 2.36-2.32 (m, 1H), 2.11- |
| | | 2.06 (m, 1H). |
| A105 | mass | ¹H NMR (400 MHz, DMSO-d₆) |
| | calcd. for | δ 10.88 (s, 1H), 9.47- |
| | C₄₀H₃₈N₈O₅, | 9.40 (m, 1H), 9.16 (d, |
| | 710.3; m/z | J = 8.5 Hz, 1H), 8.73-8.67 (m, |
| | found, 711.3 | 1H), 8.38 (s, 2H), 8.15 |
| | [M + H]⁺. | (d, J = 8.2 Hz, 1H), 7.96 (s, 1H), |
| | | 7.71 (s, 1H), 7.57 (d, J = 8.6 Hz, 1H), |
| | | 7.39-7.34 (m, 1H), |
| | | 7.07-7.05 (m, 1H), 7.00-6.97 |
| | | (m, 1H), 5.21-5.11 (m, |
| | | 1H), 4.19-4.13 (m, 1H), |
| | | 3.71-3.63 (m, 1H), 3.59 (s, |
| | | 3H), 3.40 (s, 3H), 3.31 (s, 3H), |
| | | 2.75-2.66 (m, 1H), 2.60- |
| | | 2.54 (m, 1H), 2.39-2.29 (m, 1H), |
| | | 2.12-2.05 (m, 1H), |
| | | 1.57 (d, J = 6.9 Hz, 3H), |
| | | 1.27 (d, J = 6.8 Hz, 6H). |
| A106 | mass | ¹H NMR (400 MHz, DMSO-d₆) |
| | calcd. for | δ 10.89 (s, 1H), 9.23 (d, J = |
| | C₃₉H₃₆N₈O₅, | 1.8 Hz, 1H), 9.14 (t, J = 5.9 |
| | 696.2; m/z | Hz, 1H), 8.45 (d, J = 1.6 |
| | found, 697.3 | Hz, 1H), 8.42-8.34 (m, 3H), |
| | [M + H]⁺. | 7.96 (s, 1H), 7.76-7.67 (m, |
| | | 1H), 7.63-7.54 (m, 1H), |
| | | 7.43-7.28 (m, 1H), 7.12-7.04 |
| | | (m, 1H), 7.04-6.91 (m, 1H), |
| | | 4.33 (d, J = 5.8 Hz, 2H), |
| | | 4.16 (dd, J = 12.2, 4.9 Hz, 1H), |
| | | 3.62 (s, 3H), 3.40 (s, 3H), |
| | | 2.76-2.67 (m, 1H), 2.65 (s, 3H), |
| | | 2.63-2.60 (m, 1H), |
| | | 2.59-2.53 (m, 1H), 2.38-2.27 |
| | | (m, 1H), 2.14-2.05 (m, |
| | | 1H), 1.80 (d, J = 7.6 Hz, 6H). |
| A107 | mass | ¹H NMR (400 MHz, DMSO-d₆) |
| | calcd. for | δ 10.88 (s, 1H), 9.44 (s, |
| | C₄₁H₃₃N₇O₆, | 1H), 9.35 (t, J = 5.8 Hz, 1H), |
| | 719.25; m/z | 9.15 (s, 1H), 8.85-8.72 (m, |
| | found, 720 | 2H), 8.21 (d, J = 8.2 Hz, 1H), |
| | [M + H]⁺. | 7.96 (s, 1H), 7.72 (s, 2H), |
| | | 7.61-7.55 (m, 1H), 7.40-7.34 |
| | | (m, 1H), 7.22 (s, 1H), |
| | | 7.03 (s, 1H), 4.40-4.35 (m, 2H), |
| | | 4.15 (dd, J = 12.4, 4.9 |
| | | Hz, 1H), 3.66-3.63 (m, 6H), |
| | | 3.37 (s, 3H), 2.76-2.71 (m, |
| | | 1H), 2.67 (s, 3H), 2.59-2.57 |
| | | (m, 1H), 2.35-2.31 (m, |
| | | 1H), 2.11-2.05 (m, 1H). |
| A108 | mass | ¹H NMR (400 MHz, DMSO-d₆) |
| | calcd. for | δ 10.88 (s, 1H), 9.90 (s, |
| | C₄₄H₃₈N₈O₆, | 1H), 9.49 (d, J = 1.6 Hz, 1H), |
| | 774.2; m/z | 9.34 (t, J = 5.9 Hz, 1H), |
| | found, 775.4 | 8.81 (dd, J = 8.2, 2.2 Hz, 1H), |
| | [M + H]⁺. | 8.75 (s, 1H), 8.23 (d, J = |
| | | 8.2 Hz, 1H), 8.15-8.12 (m, 1H), |
| | | 7.97-7.95 (m, 1H), |
| | | 7.94-7.89 (m, 2H), 7.73 (d, J = 1.2 Hz, |
| | | 1H), 7.61 (s, 1H), |
| | | 7.58 (d, J = 8.5 Hz, 1H), |
| | | 7.38 (dd, J = 8.5, 1.6 Hz, 1H), |
| | | 4.39 (d, J = 6.0 Hz, 2H), 4.16 |
| | | (dd, J = 12.2, 4.7 Hz, 1H), |
| | | 3.97 (q, J = 7.0 Hz, 2H), 3.84-3.80 |
| | | (m, 4H), 3.65 (s, 3H), |
| | | 3.18-3.14 (m, 4H), 2.75-2.67 |
| | | (m, 1H), 2.58-2.54 (m, |
| | | 1H), 2.38-2.30 (m, 1H), 2.13-2.07 |
| | | (m, 1H), 1.28-1.25 |
| | | (m, 3H). |
| A110 | mass | ¹H NMR (400 MHz, DMSO-d₆) |
| | calcd. for | δ 10.88 (s, 1H), 9.24 (t, J = |
| | C₄₂H₃₇FN₆O₆, | 5.8 Hz, 1H), 8.77 (s, 1H), 8.09 |
| | 740.28; | (s, 2H), 7.97-7.93 (m, |
| | m/z found, | 1H), 7.71 (s, 1H), 7.60-7.55 (m, |
| | 741.28 | 1H), 7.36 (d, J = 8.5 Hz, |
| | [M + H]⁺. | 1H), 7.16-7.12 (m, 1H), 7.07 |
| | | (s, 1H), 7.00 (s, 1H), 6.39- |
| | | 6.33 (m, 1H), 4.39-4.32 (m, |
| | | 4H), 4.18-4.13 (m, 1H), |
| | | 3.77-3.75 (m, 2H), 3.69-3.64 |
| | | (m, 1H), 3.59 (s, 3H), |
| | | 3.32 (s, 3H), 2.75-2.68 (m, |
| | | 1H), 2.60-2.54 (m, 1H), |
| | | 2.36-2.30 (m, 1H), 2.11-2.06 (m, |
| | | 1H), 1.31-1.27 (m, |
| | | 6H). |
| A111 | mass | ¹H NMR (400 MHz, DMSO-d₆) |
| | calcd. for | δ 10.88 (s, 1H), 9.12- |
| | C₄₀H₃₃FN₆O₆, | 9.03 (m, 1H), 8.57 (s, 1H), 7.96 (s, 1H), |
| | 712.2; m/z | 7.86 (s, 1H), 7.71 |
| | found, 713.4 | (s, 1H), 7.60-7.55 (m, 1H), |
| | [M + H]⁺. | 7.38-7.32 (m, 1H), 7.25- |
| | | 7.22 (m, 2H), 7.13-7.06 (m, 2H), |
| | | 6.32 (d, J = 13.2 Hz, |
| | | 1H), 4.37-4. 30 (m, 4H), |
| | | 4.18-4.13 (m, 1H), 3.77- |
| | | 3.72 (m, 2H), 3.36-3.36 (m, |
| | | 3H), 3.34 (s, 3H), 2.76- |
| | | 2.67 (m, 1H), 2.61 (s, 3H), |
| | | 2.59-2.53 (m, 1H), 2.37- |
| | | 2.29 (m, 1H), 2.12-2.05 (m, 1H). |
| A112 | mass | ¹H NMR (400 MHz, DMSO-d₆) |
| | calcd. for | δ 10.89 (s, 1H), 9.24 (d, J = |
| | C₄₃H₄₃N₉O₅, | 1.6 Hz, 1H), 9.14 (t, J = 5.8 Hz, |
| | 765.3; m/z | 1H), 8.45 (d, J = 1.4 |
| | found, 766.8 | Hz, 1H), 8.41 (s, 2H), 7.97 (s, |
| | [M + H]⁺. | 1H), 7.72 (d, J = 1.2 Hz, |
| | | 1H), 7.59 (d, J = 8.5 Hz, 1H), |
| | | 7.37 (dd, J = 8.5, 1.5 Hz, |
| | | 1H), 7.13 (d, J = 1.9 Hz, 1H), 6.89 |
| | | (s, 1H), 4.34 (d, J = 5.8 |
| | | Hz, 2H), 4.16 (dd, J = 12.2, 4.8 Hz, |
| | | 1H), 3.62 (s, 3H), 3.47- |
| | | 3.46 (m, 1H), 3.45-3.43 (m, 3H), |
| | | 3.41 (s, 3H), 3.33 (s, |
| | | 3H), 2.93-2.86 (m, 2H), 2.75-2.70 |
| | | (m, 1H), 2.66 (s, |
| | | 5H), 2.60-2.55 (m, 1H), 2.37-2.31 |
| | | (m, 1H), 2.12-2.07 |
| | | (m, 1H), 2.01-1.95 (m, 2H), |
| | | 1.90-1.81 (m, 2H). |
| A113 | mass | ¹H NMR (400 MHz, DMSO-d₆) |
| | calcd. for | δ 10.88 (s, 1H), 9.27 (t, J = |
| | C₄₂H₃₇ClN₆O₆, | 6.0 Hz, 1H), 8.67 (d, J = 1.5 Hz, |
| | 756.24; | 1H), 8.10 (d, J = 7.6 |
| | m/z found, | Hz, 1H), 8.03 (dd, J = 8.1, 2.2 Hz, |
| | 757.6 | 1H), 7.96 (s, 1H), 7.71 |
| | [M + H]⁺. | (d, J = 1.3 Hz, 1H), 7.57 (d, |
| | | J = 8.5 Hz, 1H), 7.36 (dd, J = |
| | | 8.5, 1.6 Hz, 1H), 7.07 (d, |
| | | J = 2.0 Hz, 1H), 7.01-6.96 (m, |
| | | 2H), 6.60 (s, 1H), 4.42-4.31 |
| | | (m, 4H), 4.20-4.10 (m, |
| | | 1H), 3.80-3.73 (m, 2H), |
| | | 3.71-3.63 (m, 1H), 3.59 (s, |
| | | 3H), 3.33 (s, 3H), 2.74-2.67 |
| | | (m, 1H), 2.61-2.54 (m, |
| | | 1H), 2.37-2.30 (m, 1H), 2.11-2.05 |
| | | (m, 1H), 1.29 (d, J = |
| | | 6.8 Hz, 6H). |
| A114 | mass | ¹H NMR (400 MHz, DMSO-d₆) |
| | calcd. for | δ 10.89 (s, 1H), 9.25 (d, J = |
| | C₄₁H₃₇N₇O₇, | 2.0 Hz, 1H), 9.16 (s, 1H), 8.47 |
| | 739.3; m/z | (dd, J = 2.1, 0.8 Hz, 1H), |
| | found, 740.3 | 8.45 (s, 2H), 7.96 (s, 1H), |
| | [M + H]⁺. | 7.72 (d, J = 1.6 Hz, 1H), 7.59 |
| | | (d, J = 8.5 Hz, 1H), 7.37 |
| | | (dd, J = 8.6, 1.7 Hz, 1H), 7.21 (d, |
| | | J = 2.1 Hz, 1H), 7.04 (d, J = 2.2 |
| | | Hz, 1H), 4.33 (d, J = 5.9 |
| | | Hz, 2H), 4.16 (dd, |
| | | J = 12.2, 4.9 Hz, 1H), 3.99-3.91 (m, |
| | | 2H), 3.41 (s, 3H), 3.33 (s, 3H), |
| | | 3.12-3.00 (m, 1H), 2.77- |
| | | 2.68 (m, 2H), 2.66 (s, 3H), |
| | | 2.61-2.54 (m, 1H), 2.40- |
| | | 2.28 (m, 1H), 2.13-2.06 |
| | | (m, 1H), 2.04-1.96 (m, 1H), |
| | | 1.91-1.80 (m, 2H), 1.78-1.63 (m, 2H). |
| A115 | mass | ¹H NMR (400 MHz, DMSO-d₆) |
| | calcd. for | δ 10.89 (s, 1H), 9.90 (s, |
| | C₄₂H₃₄N₈O₆, | 1H), 9.52-9.44 (m, 1H), 9.34 |
| | 746.3; m/z | (t, J = 5.9 Hz, 1H), 8.81 |
| | found, 747.2 | (dd, J = 8.3, 2.0 Hz, 1H), 8.75 |
| | [M + H]⁺. | (s, 1H), 8.23 (d, J = 8.2 Hz, |
| | | 1H), 8.12 (d, J = 7.9 Hz, 1H), 7.96 |
| | | (s, 1H), 7.92 (t, J = 7.6 |
| | | Hz, 1H), 7.87 (d, J = 7.0 Hz, 1H), |
| | | 7.73 (s, 1H), 7.58 (d, J = |
| | | 8.5 Hz, 1H), 7.40-7.35 (m, |
| | | 1H), 7.33 (s, 1H), 5.73- |
| | | 5.48 (m, 1H), 4.61-4.54 (m, 1H), |
| | | 4.39 (d, J = 5.9 Hz, |
| | | 2H), 4.29 (t, J = 7.6 Hz, 2H), |
| | | 4.16 (dd, J = 12.3, 4.9 Hz, |
| | | 1H), 3.98-3.91 (m, 2H), 3.56 (s, 3H), |
| | | 3.40 (s, 3H), 2.76- |
| | | 2.67 (m, 1H), 2.58-2.53 (m, 1H), |
| | | 2.39-2.29 (m, 1H), |
| | | 2.12-2.05 (m, 1H). |
| A116 | mass | ¹H NMR (400 MHz, DMSO-d₆) |
| | calcd. for | δ 10.89 (s, 1H), 9.14 (t, J = |
| | C₄₁H₃₈ClN₇O₅, | 5.9 Hz, 1H), 8.73 (d, J = 1.8 Hz, 1H), |
| | 743.2; | 8.08 (d, J = 2.5 |
| | m/z found, | Hz, 1H), 8.01-7.98 (m, 1H), 7.96 |
| | 744.3 | (s, 1H), 7.74-7.70 (m, |
| | [M + H]⁺. | 1H), 7.60-7.57 (m, 1H), 7.40-7.33 |
| | | (m, 1H), 7.21 (d, J = |
| | | 2.5 Hz, 1H), 7.05-7.01 (m, 1H), |
| | | 6.98-6.93 (m, 1H), |
| | | 4.33 (d, J = 5.8 Hz, 2H), 4.16 |
| | | (dd, J = 12.2, 4.8 Hz, 1H), |
| | | 3.69-3.64 (m, 1H), 3.59 (s, |
| | | 3H), 3.37 (s, 3H), 3.31 (s, |
| | | 3H), 2.76-2.68 (m, 1H), 2.62 |
| | | (s, 3H), 2.59-2.54 (m, |
| | | 1H), 2.39-2.29 (m, 1H), 2.13-2.06 |
| | | (m, 1H), 1.27 (d, J = |
| | | 6.8 Hz, 6H). |
| A117 | mass | ¹H NMR (400 MHz, DMSO-d₆) |
| | calcd. for | δ 10.88 (s, 1H), 9.22 (t, J = |
| | C₄₃H₃₉FN₆O₅, | 5.9 Hz, 1H), 8.75 (s, 1H), |
| | 738.3; m/z | 8.08 (s, 2H), 7.96 (s, 1H), |
| | found, 739.3 | 7.71 (s, 1H), 7.57 (d, |
| | [M + H]⁺. | J = 8.5 Hz, 1H), 7.39-7.31 (m, 2H), |
| | | 7.01 (d, J = 1.6 Hz, 1H), 6.94 |
| | | (d, J = 1.6 Hz, 1H), 6.07 (d, |
| | | J = 14.6 Hz, 1H), 4.36 (d, |
| | | J = 5.8 Hz, 2H), 4.15 (dd, J = |
| | | 12.2, 4.8 Hz, 1H), 3.65 (dd, J = 13.9, |
| | | 6.3 Hz, 3H), 3.59 (s, |
| | | 3H), 3.32 (s, 3H), 2.91-2.85 |
| | | (m, 2H), 2.75-2.70 (m, |
| | | 1H), 2.61-2.54 (m, 1H), 2.38-2.31 |
| | | (m, 1H), 2.11-2.01 |
| | | (m, 3H), 1.28 (d, J = 6.8 Hz, 6H). |
| A118 | mass | ¹H NMR (400 MHz, DMSO-d₆) |
| | calcd. for | δ 10.75 (s, 1H), 9.69 (s, |
| | C₄₅H₄₂N₈O₅Si, | 1H), 9.39-9.33 (m, 1H), 9.21 (t, |
| | 802.3; | J = 5.9 Hz, 1H), 8.71- |
| | m/z found, | 8.64 (m, 1H), 8.62 (s, 1H), 8.10 |
| | 803.4 | (d, J = 8.2 Hz, 1H), 8.00 |
| | [M + H]⁺. | (d, J = 7.9 Hz, 1H), 7.83 (s, 1H), |
| | | 7.80 (t, J = 7.8 Hz, 1H), |
| | | 7.74 (d, J = 7.2 Hz, 1H), 7.60 (s, 1H), |
| | | 7.45 (d, J = 8.5 Hz, |
| | | 1H), 7.37 (s, 1H), 7.25 (d, J = 8.7 Hz, |
| | | 1H), 4.26 (d, J = 5.8 |
| | | Hz, 2H), 4.04-4.02 (m, 1H), |
| | | 3.53 (s, 3H), 3.31-3.25 (m, |
| | | 7H), 2.62-2.54 (m, 1H), 2.47-2.41 |
| | | (m, 1H), 2.25-2.18 |
| | | (m, 1H), 1.98-1.94 (m, 1H), |
| | | 0.84-0.77 (m, 4H), 0.00 (s, |
| | | 6H). |
| A119 | mass | ¹H NMR (400 MHz, DMSO-d₆) |
| | calcd. for | δ 10.88 (s, 1H), 9.54- |
| | C₄₀H₃₀N₆O₅, | 9.47 (m, 1H), 9.43-9.38 (m, |
| | 674.2; m/z | 1H), 9.34 (t, J = 6.0 Hz, |
| | found, 675.3 | 1H), 8.80 (dd, J = 8.2, 2.2 Hz, 1H), |
| | [M + H]⁺. | 8.77 (s, 1H), 8.22 (d, J = |
| | | 8.2 Hz, 1H), 8.13-8.07 (m, 1H), |
| | | 7.96 (s, 1H), 7.94- |
| | | 7.89 (m, 1H), 7.74-7.71 (m, 1H), |
| | | 7.69-7.65 (m, 1H), |
| | | 7.60-7.55 (m, 1H), 7.47-7.43 |
| | | (m, 1H), 7.39-7.29 (m, |
| | | 3H), 4.38 (d, J = 5.9 Hz, 2H), |
| | | 4.16 (dd, J = 12.3, 4.9 Hz, |
| | | 1H), 3.43 (s, 3H), 3.40 (s, 3H), |
| | | 2.76-2.67 (m, 1H), 2.60- |
| | | 2.54 (m, 1H), 2.37-2.30 |
| | | (m, 1H), 2.13-2.05 (m, 1H). |
| A120 | mass | ¹H NMR (400 MHz, DMSO-d₆) |
| | calcd. for | δ 10.94-10.83 (m, 1H), |
| | C₃₉H₂₇N₅O₆, | 9.50 (d, J = 1.6 Hz, 1H), 9.40-9.31 |
| | 661.20; m/z | (m, 2H), 8.79 (dd, J = |
| | found, 662.4 | 8.8, 2.8 Hz, 2H), 8.23 |
| | [M + H]⁺. | (d, J = 8.2 Hz, 1H), 8.12 (d, J = |
| | | 8.2 Hz, 1H), 7.98-7.89 (m, 2H), |
| | | 7.72 (s, 1H), 7.69-7.64 |
| | | (m, 2H), 7.60-7.56 (m, 1H), |
| | | 7.51-7.45 (m, 2H), 7.40- |
| | | 7.35 (m, 1H), 4.38 (d, J = 6.0 Hz, |
| | | 2H), 4.16 (dd, J = 12.4, |
| | | 4.8 Hz, 1H), 3.45 (s, 3H), 2.75-2.67 |
| | | (m, 1H), 2.60-2.53 |
| | | (m, 1H), 2.38-2.29 (m, 1H), |
| | | 2.12-2.03 (m, 1H). |
| A121 | mass | ¹H NMR (400 MHz, DMSO-d₆) |
| | calcd. for | δ 10.88 (s, 1H), 9.18 (s, |
| | C₄₂H₃₈N₆O₆, | 1H), 8.88 (d, J = 2.1 Hz, 1H), |
| | 722.2; m/z | 8.22-8.14 (m, 1H), 8.04 |
| | found, 723.2 | (d, J = 8.3 Hz, 1H), 7.96 (s, 1H), |
| | [M + H]⁺. | 7.71 (s, 1H), 7.57 (d, J = |
| | | 8.5 Hz, 1H), 7.40-7.33 (m, 1H), |
| | | 7.32 (d, J = 2.1 Hz, 1H), |
| | | 7.21-7.16 (m, 1H), 7.02 (d, |
| | | J = 2.0 Hz, 1H), 6.96 (d, J = |
| | | 1.9 Hz, 1H), 6.69 (d, J = 8.5 |
| | | Hz, 1H), 4.42-4.31 (m, 4H), |
| | | 4.15 (dd, J = 12.2, 4.9 Hz, 1H), |
| | | 3.75 (d, J = 4.0 Hz, 2H), |
| | | 3.69-3.62 (m, 1H), 3.58 (s, 3H), |
| | | 3.31 (s, 3H), 2.76-2.67 |
| | | (m, 1H), 2.59-2.53 (m, 1H), |
| | | 2.38-2.29 (m, 1H), 2.12- |
| | | 2.04 (m, 1H), 1.28 (d, J = 6.8 Hz, 6H). |
| A122 | mass | ¹H NMR (400 MHz, DMSO-d₄) |
| | calcd. for | δ 10.88 (s, 1H), 9.38 (s, |
| | C₃₈H₃₃N₉O₅, | 1H), 9.31-9.25 (m, 1H), 8.71-8.64 |
| | 695.3; m/z | (m, 1H), 8.17-8.13 |
| | found, 696.3 | (m, 1H), 7.96 (s, 1H), 7.71 |
| | [M + H]⁺. | (s, 1H), 7.57 (d, J = 8.6 Hz, |
| | | 1H), 7.39-7.33 (m, 2H), |
| | | 7.25-7.21 (m, 2H), 7.05 (d, J = |
| | | 8.5 Hz, 1H), 4.36 (d, J = 5.7 |
| | | Hz, 2H), 4.15 (dd, J = 12.3, |
| | | 5.0 Hz, 1H), 3.74 (s, 3H), |
| | | 3.36 (s, 3H), 3.35-3.32 (m, |
| | | 7H), 2.72-2.66 (m, 1H), 2.59-2.55 |
| | | (m, 1H), 2.35-2.30 |
| | | (m, 1H), 2.11-2.05 (m, 1H). |
| A123 | mass | ¹H NMR (400 MHz, DMSO-d₆) |
| | calcd. for | δ 10.88 (s, 1H), 9.76 (s, |
| | C₄₄H₃₇N₇O₇S, | 1H), 9.48 (d, J = 1.7 Hz, 1H), |
| | 807.25; | 9.34 (t, J = 5.9 Hz, 1H), |
| | m/z found, | 8.83-8.73 (m, 2H), 8.23 |
| | 808 [M + H]⁺. | (d, J = 8.3 Hz, 1H), 8.15 (d, J = |
| | | 8.2 Hz, 1H), 7.98-7.93 (m, 2H), |
| | | 7.90-7.86 (m, 1H), |
| | | 7.73 (d, J = 1.2 Hz, 1H), 7.66 |
| | | (s, 1H), 7.59-7.57 (m, 1H), |
| | | 7.38 (dd, J = 8.6, |
| | | 1.5 Hz, 1H), 4.44-4.36 (m, 2H), 4.16 |
| | | (dd, J = 12.2, 4.9 Hz, 1H), |
| | | 3.85-3.76 (m, 1H), 3.69 (s, |
| | | 3H), 3.54-3.47 (m, 2H), 3.44 |
| | | (s, 3H), 3.18-3.12 (m, |
| | | 2H), 2.75-2.67 (m, 1H), |
| | | 2.59-2.54 (m, 1H), 2.46-2.39 |
| | | (m, 2H), 2.35-2.24 (m, 3H), |
| | | 2.11-2.06 (m, 1H). |
| A124 | mass | ¹H NMR (400 MHz, DMSO-d₆) |
| | calcd. for | δ 10.88 (s, 1H), 9.87 (s, |
| | C₄₄H₃₈N₈O₆, | 1H), 9.49 (d, J = 1.8 Hz, 1H), |
| | 774.29; m/z | 9.33 (t, J = 5.9 Hz, 1H), |
| | found, 775.3 | 8.80 (dd, J = 8.2, 2.1 Hz, 1H), |
| | [M + H]⁺. | 8.74 (s, 1H), 8.23 (d, J = |
| | | 8.2 Hz, 1H), 8.12 (d, J = 8.0 Hz, |
| | | 1H), 7.95 (d, J = 6.8 Hz, |
| | | 1H), 7.89 (dd, J = 15.9, 7.0 Hz, |
| | | 2H), 7.73 (d, J = 1.1 Hz, |
| | | 1H), 7.58 (d, J = 8.5 Hz, |
| | | 1H), 7.50 (s, 1H), 7.38 (dd, J = |
| | | 8.6, 1.5 Hz, 1H), 4.70 (d, J = 3.9 Hz, |
| | | 1H), 4.39 (d, J = 5.9 |
| | | Hz, 2H), 4.16 (dd, J = 12.2, 4.8 Hz, |
| | | 1H), 3.63 (s, 3H), 3.44- |
| | | 3.39 (m, 4H), 3.39-3.34 (m, |
| | | 2H), 2.93 (t, J = 10.0 Hz, |
| | | 2H), 2.75-2.67 (m, 1H), 2.60-2.52 |
| | | (m, 1H), 2.38-2.29 |
| | | (m, 1H), 2.12-2.05 (m, 1H), |
| | | 1.94-1.87 (m, 2H), 1.67- |
| | | 1.57(m, 2H). |
| A125 | mass | ¹H NMR (400 MHz, DMSO-d₆) |
| | calcd. for | δ 10.89 (s, 1H), 9.15- |
| | C₄₀H₃₇N₇O₅, | 9.01 (m, 2H), 8.57 (s, 1H), 8.43 |
| | 695.8; m/z | (d, J = 2.8 Hz, 1H), 8.30 |
| | found, 696.1 | (d, J = 2.0 Hz, 1H), 8.03-7.84 |
| | [M + H]⁺. | (m, 2H), 7.72 (d, J = 1.6 |
| | | Hz, 1H), 7.58 (d, J = 8.5 Hz, 1H), |
| | | 7.47 (dd, J = 8.8, 2.8 |
| | | Hz, 1H), 7.37 (dd, J = 8.5, 1.7 Hz, |
| | | 1H), 6.87 (d, J = 2.1 |
| | | Hz, 1H), 6.81 (d, |
| | | J = 2.1 Hz, 1H), 4.40-4.16 (m, 2H), |
| | | 4.19-4.01 (m, 1H), 3.70-3.58 |
| | | (m, 1H), 3.56 (s, 3H), |
| | | 3.30 (s, 3H), 2.79-2.63 (m, 4H), 2.34 |
| | | (qd, J = 12.8, 4.7 |
| | | Hz, 1H), 2.04 (ddd, J = 37.5, |
| | | 13.6, 7.6 Hz, 2H), 1.28 (d, J = |
| | | 6.8 Hz, 7H). |
| A126 | mass | ¹H NMR (400 MHz, DMSO-d₆) |
| | calcd. for | δ 10.89 (s, 1H), 9.18- |
| | C₃₇H₃₁N₉O₆, | 9.13 (m, 2H), 8.39 (s, 1H), 8.07 |
| | 697.2; m/z | (d, J = 2.1 Hz, 1H), 7.98 |
| | found, 698.2 | (s, 1H), 7.97-7.95 (m, |
| | [M + H]⁺. | 1H), 7.73-7.71 (m, 2H), 7.59- |
| | | 7.57 (m, 1H), 7.38-7.35 |
| | | (m, 1H), 4.66-4.62 (m, 2H), |
| | | 4.32 (d, J = 5.8 Hz, 2H), |
| | | 4.18-4.14 (m, 1H), 3.83-3.80 |
| | | (m, 2H), 3.38 (s, 3H), |
| | | 3.35 (s, 3H), 2.70-2.66 (m, 1H), |
| | | 2.64 (s, 3H), 2.55-2.53 (m, |
| | | 1H), 2.35-2.32 (m, 1H), |
| | | 2.11-2.06 (m, 1H). |
| A127 | mass | ¹H NMR (400 MHz, DMSO-d₆) |
| | calcd. for | δ 10.88 (s, 1H), 9.38 (s, |
| | C₄₂H₃₉N₉O₆, | 1H), 9.30-9.25 (m, 1H), |
| | 765.3; m/z | 8.70-8.64 (m, 1H), 8.18-8.12 |
| | found, 766.7 | (m, 1H), 7.96 (s, 1H), |
| | [M + H]⁺. | 7.94-7.91 (m, 1H), 7.71 (s, 1H), |
| | | 7.59-7.55 (m, 1H), 7.39-7.34 |
| | | (m, 1H), 7.04 (s, 1H), |
| | | 6.93 (s, 1H), 4.38-4.33 |
| | | (m, 2H), 4.18-4.12 (m, 1H), |
| | | 3.83-3.73 (m, 4H), 3.71-3.67 |
| | | (m, 2H), 3.64 (s, 3H), |
| | | 3.31 (s, 3H), 3.07-3.02 (m, 2H), |
| | | 2.96-2.89 (m, 4H), |
| | | 2.75-2.67 (m, 1H), 2.59-2.54 |
| | | (m, 1H), 2.36-2.30 (m, |
| | | 1H), 2.18-2.06 (m, 3H). |
| A128 | mass | ¹H NMR (400 MHz, DMSO-d₆) |
| | calcd. for | δ 10.88 (s, 1H), 9.09 (t, J = |
| | C₄₂H₄₀N₈O₆, | 5.8 Hz, 1H), 9.03 (s, 1H), 8.91 |
| | 752.3; m/z | (s, 1H), 8.27 (s, 1H), |
| | found, 753.3 | 7.96 (s, 1H), 7.92 (s, 1H), 7.71 |
| | [M + H]⁺. | (s, 1H), 7.58 (d, J = 8.5 |
| | | Hz, 1H), 7.37 (d, J = 8.5 Hz, |
| | | 1H), 7.16 (d, J = 1.8 Hz, |
| | | 1H), 7.06 (d, J = 1.8 Hz, 1H), |
| | | 4.33 (d, J = 5.8 Hz, 2H), |
| | | 4.20-3.92 (m, 4H), 3.63 (s, 3H), |
| | | 3.32 (s, 3H), 2.83-2.52 |
| | | (m, 8H), 2.34 (m, 2H), 2.12-2.06 |
| | | (m, 1H), 1.79-1.71 |
| | | (m, 5H). |
| A129 | mass | ¹H NMR (400 MHz, DMSO-d₆) |
| | calcd. for | δ 10.88 (s, 1H), 9.44 (s, |
| | C₄₀H₃₁N₇O₆, | 1H), 9.35 (d, J = 5.6 Hz, 1H), 9.13 |
| | 705.23; m/z | (s, 1H), 8.78 (dd, J = |
| | found, 706 | 11.1, 2.5 Hz, 2H), 8.21 |
| | [M + H]⁺. | (d, J = 8.3 Hz, 1H), 7.96 (s, 1H), |
| | | 7.73 (d, J = 6.7 Hz, 2H), 7.58 (d, |
| | | J = 8.5 Hz, 1H), 7.37 (d, |
| | | J = 10.6 Hz, 2H), 7.32 (d, |
| | | J = 8.0 Hz, 1H), 7.26 (d, J = 9.3 |
| | | Hz, 1H), 4.41-4.35 (m, 2H), |
| | | 4.15 (dd, J = 12.2, 4.8 Hz, |
| | | 1H), 3.66 (s, 3H), 3.41 (s, 3H), |
| | | 3.39 (s, 3H), 2.74-2.71 |
| | | (m, 1H), 2.59-2.57 (m, 1H), |
| | | 2.34-2.32 (m, 1H), 2.01- |
| | | 1.98 (m, 1H). |
| A130 | mass | ¹H NMR (400 MHz, DMSO-d₆) |
| | calcd. for | δ 10.89 (s, 1H), 9.50 (d, J = |
| | C₄₂H₃₃N₅O₅, | 2.1 Hz, 1H), 9.36 (s, 2H), |
| | 687.8; m/z | 8.84-8.72 (m, 1H), 8.23 (d, J = |
| | found, 688.8 | 8.2 Hz, 1H), 8.11 |
| | [M + H]⁺. | (d, J = 8.2 Hz, 1H), 8.01-7.89 (m, |
| | | 1H), 7.72 (d, J = 1.6 Hz, 1H), |
| | | 7.66 (d, J = 7.5 Hz, 1H), |
| | | 7.58 (d, J = 8.6 Hz, 1H), 7.55-7.51 |
| | | (m, 2H), 7.38 (dd, J = |
| | | 8.6, 1.7 Hz, 1H), 7.24 (d, |
| | | J = 7.9 Hz, 1H), 4.39 (d, J = |
| | | 5.9 Hz, 2H), 4.16 (dd, J = 12.3, |
| | | 4.9 Hz, 1H), 3.24 (s, 3H), |
| | | 2.75-2.66 (m, 1H), 2.63-2.56 |
| | | (m, 1H), 2.42-2.28 (m, |
| | | 1H), 2.17-2.05 (m, 1H), 1.37 (s, 6H). |
| A132 | mass | ¹H NMR (400 MHz, DMSO-d₆) |
| | calcd. for | δ 10.88 (s, 1H), 9.38 (d, J = |
| | C₃₈H₃₂N₈O₅, | 1.4 Hz, 1H), 9.28 (t, J = 6.0 Hz, |
| | 680.25; m/z | 1H), 8.66 (dd, J = 8.2, |
| | found, 681 | 2.1 Hz, 1H), 8.14 (d, J = 7.7 Hz, |
| | [M + H]⁺. | 1H), 7.96 (s, 1H), 7.88 (s, |
| | | 1H), 7.71 (d, J = 1.3 Hz, 1H), |
| | | 7.57 (d, J = 8.5 Hz, 1H), |
| | | 7.36 (dd, J = 8.5, 1.6 Hz, 1H), |
| | | 7.25 (t, J = 4.8 Hz, 2H), |
| | | 7.08 (dd, J = 8.2, 2.0 Hz, |
| | | 1H), 4.35 (d, J = 6.0 Hz, 2H), |
| | | 4.15 (dd, J = 12.3, 4.7 Hz, 1H), |
| | | 3.71-3.65 (m, 2H), 3.36 |
| | | (s, 3H), 3.33 (s, 3H), 3.05 (t, J = 6.5 Hz, |
| | | 2H), 2.69 (dd, J = |
| | | 12.0, 5.2 Hz, 1H), 2.56 |
| | | (d, J = 17.2 Hz, 1H), 2.36-2.29 |
| | | (m, 1H), 2.19-2.13 (m, 2H), |
| | | 2.10-2.03 (m, 1H). |
| A133 | mass | ¹H NMR (400 MHz, DMSO-d₆) |
| | calcd. for | δ 10.88 (s, 1H), 9.38 (d, J = |
| | C₄₁H₃₈N₈O₅, | 1.4 Hz, 1H), 9.28 (t, J = 5.9 Hz, |
| | 722.8; m/z | 1H), 8.67 (dd, J = 8.2, |
| | found, 723.4 | 2.1 Hz, 1H), 8.13 (d, J = 8.2 Hz, |
| | [M + H]⁺. | 1H), 7.96 (s, 1H), 7.88 (s, |
| | | 1H), 7.71 (d, J = 0.9 Hz, 1H), |
| | | 7.57 (d, J = 8.5 Hz, 1H), |
| | | 7.36 (dd, J = 8.6, 1.5 Hz, 1H), |
| | | 7.08 (d, J = 1.9 Hz, 1H), |
| | | 7.01 (d, J = 1.8 Hz, 1H), |
| | | 4.36 (d, J = 5.9 Hz, 2H), 4.15 |
| | | (dd, J = 12.2, 4.8 Hz, 1H), |
| | | 3.71-3.62 (m, 3H), 3.60 (s, |
| | | 3H), 3.31 (s, 3H), 3.04 |
| | | (t, J = 6.4 Hz, 2H), 2.76-2.67 (m, |
| | | 1H), 2.59-2.53 (m, 1H), 2.38-2.29 |
| | | (m, 1H), 2.20-2.13 |
| | | (m, 2H), 2.11-2.03 (m, 1H), |
| | | 1.28 (d, J = 6.8 Hz, 6H). |
| A134 | mass | ¹H NMR (400 MHz, DMSO-d₆) |
| | calcd. for | δ 10.88 (s, 1H), 9.33 (s, |
| | C₄₃H₃₇N₇O₆, | 1H), 8.81 (s, 1H), 8.15 (s, 2H), |
| | 747.28; m/z | 7.95 (s, 1H), 7.71 (s, 1H), |
| | found, 748.28 | 7.57 (d, J = 8.1 Hz, 1H), |
| | [M + H]⁺. | 7.36 (d, J = 8.4 Hz, 1H), 7.21 (s, |
| | | 1H), 7.08 (s, 1H), 7.01 (s, 1H), |
| | | 6.86 (s, 1H), 4.48 (s, 2H), |
| | | 4.36 (d, J = 5.9 Hz, 2H), 4.18-4.12 |
| | | (m, 1H), 3.79 (s, 2H), |
| | | 3.60 (s, 3H), 3.33 (s, 3H), |
| | | 2.69-2.64 (m, 1H), 2.59-2.54 |
| | | (m, 2H), 2.36-2.41 (m, 2H), |
| | | 1.29 (d, J = 6.8 Hz, 6H). |
| A135 | mass | ¹H NMR (400 MHz, DMSO-d₆) |
| | calcd. for | δ 10.88 (s, 1H), 9.87 (s, |
| | C₄₄H₃₇N₇O₆, | 1H), 9.49 (d, J = 2.0 Hz, 1H), |
| | 759.3; m/z | 9.33-9.29 (m, 1H), 8.81- |
| | found, 760.6 | 8.78 (m, 1H), 8.76 (s, 1H), |
| | [M + H]⁺. | 8.23-8.21 (m, 1H), 8.14- |
| | | 8.10 (m, 1H), 7.97-7.89 (m, 3H), |
| | | 7.73 (s, 1H), 7.64 (s, |
| | | 1H), 7.58 (d, J = 8.5 Hz, 1H), |
| | | 7.38 (d, J = 8.6 Hz, 1H), |
| | | 4.39 (d, J = 5.9 Hz, 2H), |
| | | 4.16 (dd, J = 12.3, 4.9 Hz, 1H), |
| | | 3.97 (d, J = 6.7 Hz, 2H), 3.68 |
| | | (s, 3H), 3.59-3.53 (m, 3H), |
| | | 3.44 (s, 3H), 2.70-2.65 (m, 1H), |
| | | 2.58 (s, 1H), 2.39-2.30 |
| | | (m, 1H), 2.12-2.01 (m, 3H), |
| | | 1.82-1.80 (m, 2H). |
| A136 | mass | ¹H NMR (400 MHz, DMSO-d₆) |
| | calcd. for | δ 10.88 (s, 1H), 9.84 (s, |
| | C₄₄H₃₆N₈O₆, | 1H), 9.50 (d, J = 2.2 Hz, 1H), |
| | 772.3; m/z | 9.34 (t, J = 5.9 Hz, 1H), |
| | found, 773.3 | 8.81 (dd, J = 8.2, 2.3 Hz, |
| | [M + H]⁺. | 1H), 8.75 (s, 1H), 8.23 (d, J = |
| | | 8.2 Hz, 1H), 8.12 (d, J = 8.1 Hz, |
| | | 1H), 8.00-7.75 (m, 3H), |
| | | 7.73 (d, J = 1.6 Hz, 1H), 7.58 |
| | | (d, J = 8.5 Hz, 1H), 7.42- |
| | | 7.28 (m, 2H), 4.73 (s, 3H), |
| | | 4.39 (d, J = 5.9 Hz, 2H), 4.34 |
| | | (s, 3H), 4.16 (dd, J = 12.3, |
| | | 4.9 Hz, 1H), 3.59-3.54 (m, |
| | | 4H), 3.47-3.37 (m, 4H), |
| | | 2.78-2.64 (m, 1H), 2.38-2.28 |
| | | (m, 1H), 2.05-1.95 (m, 2H). |
| A137 | mass | ¹H NMR (400 MHz, DMSO-d₆) |
| | calcd. for | δ 10.88 (s, 1H), 9.11 (t, J = |
| | C₄₂H₄₀FN₇O₅, | 5.9 Hz, 1H), 8.88 (d, J = 2.1 Hz, |
| | 741.3; m/z | 1H), 8.12 (s, 1H), 7.96 |
| | found, 742.3 | (s, 1H), 7.92 (t, J = 2.2 Hz, |
| | [M + H]⁺. | 1H), 7.71 (d, J = 1.6 Hz, 1H), |
| | | 7.58 (d, J = 8.6 Hz, 1H), 7.37 |
| | | (dd, J = 8.6, 1.7 Hz, 1H), |
| | | 7.12-6.93 (m, 2H), 6.90 (d, |
| | | J = 2.0 Hz, 1H), 4.32 (d, J = |
| | | 5.9 Hz, 2H), 4.16 (dd, |
| | | J = 4.9 Hz, 2H), 3.89-3.75 (m, |
| | | 1H), 3.72-3.60 (m, 1H), 3.60 |
| | | (s, 3H), 3.32 (s, 3H), 2.76- |
| | | 2.65 (m, 1H), 2.63 (s, 3H), |
| | | 2.38-2.23 (m, 1H), 2.15- |
| | | 1.95 (m, 2H), 1.38-0.99 (m, 9H). |
| A138 | mass | ¹H NMR (400 MHz, DMSO-d₆) |
| | calcd. for | δ 10.88 (s, 1H), 9.88 (s, |
| | C₄₃H₃₆N₈O₆, | 1H), 9.50-9.47 (m, 1H), 9.33 |
| | 760.28; m/z | (t, J = 6.0 Hz, 1H), 8.81 |
| | found, 761.4 | (dd, J = 8.2, 2.2 Hz, 1H), 8.74 |
| | [M + H]⁺. | (s, 1H), 8.22 (d, J = 8.3 Hz, |
| | | 1H), 8.12 (d, J = 7.9 Hz, 1H), |
| | | 7.96 (s, 1H), 7.95-7.87 (m, |
| | | 2H), 7.73 (d, J = 1.3 Hz, |
| | | 1H), 7.58 (d, J = 8.6 Hz, 1H), |
| | | 7.55 (s, 1H), 7.38 (dd, |
| | | J = 8.6, 1.6 Hz, 1H), 4.39 (d, J = |
| | | 5.9 Hz, 2H), 4.16 (dd, J = 12.2, |
| | | 4.8 Hz, 1H), 3.84-3.79 |
| | | (m, 4H), 3.64 (s, 3H), 3.42 (s, 3H), |
| | | 3.18-3.13 (m, 4H), |
| | | 2.76-2.67 (m, 1H), 2.59-2.53 |
| | | (m, 1H), 2.39-2.29 (m, |
| | | 1H), 2.12-2.05 (m, 1H). |
| A139 | mass | ¹H NMR (400 MHz, DMSO-d₆) |
| | calcd. for | δ 10.91-10.84 (m, 1H), |
| | C₃₉H₃₄N₈O₅, | 9.38 (t, J = 5.9 Hz, 1H), 9.10 (d, |
| | 694.3; m/z | J = 1.7 Hz, 1H), 8.46 (dd, |
| | found, 695.4 | J = 8.2, 2.0 Hz, 1H), |
| | [M + H]⁺. | 8.25 (d, J = 8.2 Hz, 1H), 7.97 (s, |
| | | 1H), 7.71 (s, 1H), 7.58 (d, J = 8.5 Hz, |
| | | 1H), 7.36 (d, J = 8.6 |
| | | Hz, 1H), 7.32 (s, 1H), 7.29-7.26 |
| | | (m, 2H), 7.15 (s, 1H), |
| | | 7.10-7.07 (m, 1H), 4.38 |
| | | (d, J = 5.9 Hz, 2H), 4.15 (dd, J = |
| | | 12.2, 4.8 Hz, 1H), 3.83-3.81 |
| | | (m, 2H), 3.76-3.75 (m, |
| | | 2H), 3.38-3.34 (m, 9H), |
| | | 2.77-2.67 (m, 1H), 2.59-2.53 |
| | | (m, 1H), 2.38-2.28 (m, 1H), |
| | | 2.12-2.05 (m, 1H). |
| A140 | mass | ¹H NMR (400 MHz, DMSO-d₆) |
| | calcd. For | δ 10.88 (s, 1H), 9.32 (t, J = |
| | C₄₅H₄₁F₂N₇O₇S, | 5.8 Hz, 1H), 8.62 (s, 1H), 8.13 |
| | 861.28; | (d, J = 8.1 Hz, 1H), 7.99- |
| | m/z found, | 7.93 (m, 2H), 7.71 (s, 1H), |
| | 862 [M + H]⁺. | 7.58 (d, J = 8.6 Hz, 1H), 7.53 |
| | | (s, 1H), 7.36 (d, J = 8.5 Hz, 1H), |
| | | 7.20 (s, 1H), 7.00 (s, |
| | | 1H), 6.76 (t, J = 54.7 Hz, 1H), |
| | | 4.37 (d, J = 5.9 Hz, 2H), |
| | | 4.15 (dd, J = 12.3, |
| | | 4.6 Hz, 1H), 3.86-3.79 (m, 2H), 3.65- |
| | | 3.57 (m, 4H), 3.47-3.38 (m, 3H), |
| | | 3.34 (s, 3H), 3.09- |
| | | 3.02 (m, 2H), 2.91-2.85 (m, 2H), |
| | | 2.75-2.67 (m, 1H), |
| | | 2.61-2.53 (m, 1H), 2.38-2.29 |
| | | (m, 3H), 2.18-2.12 (m, |
| | | 2H), 2.04-1.99 (m, 2H). |
| A141 | mass | ¹H NMR (400 MHz, DMSO-d₆) |
| | calcd. for | δ 10.88 (s, 1H), 9.08 (s, |
| | C₄₁H₃₉N₇O₅, | 1H), 9.04 (d, J = 1.8 Hz, 1H), |
| | 709.30; m/z | 8.27 (d, J = 1.6 Hz, 1H), |
| | found, 710.30 | 8.18 (d, J = 2.9 Hz, 1H), |
| | [M + H]⁺. | 7.97-7.91 (m, 2H), 7.72 (d, J = |
| | | 1.3 Hz, 1H), 7.58 (d, J = 8.6 Hz, |
| | | 1H), 7.38 (d, J = 1.6 Hz, |
| | | 1H), 7.18 (dd, J = 8.9, 3.0 Hz, |
| | | 1H), 6.99 (d, J = 2.0 Hz, |
| | | 1H), 6.92 (d, J = 2.0 Hz, |
| | | 1H), 4.32 (d, J = 5.9 Hz, 2H), |
| | | 4.16 (dd, J = 12.2, 4.8 Hz, 1H), |
| | | 3.65 (dt, J = 13.9, 6.9 Hz, |
| | | 1H), 3.59 (s, 3H), 3.37 (s, 3H), |
| | | 3.30 (s, 3H), 2.77-2.68 |
| | | (m, 1H), 2.64 (s, 3H), 2.60-2.55 |
| | | (m, 1H), 2.36-2.32 (m, |
| | | 1H), 2.12-2.06 (m, 1H), |
| | | 1.27 (d, J = 6.8 Hz, 6H). |
| A142 | mass | ¹H NMR (400 MHz, DMSO-d₆) |
| | calcd. for | δ 10.88 (s, 1H), 9.19 (t, J = |
| | C₄₃H₃₈F₂N₆O₅, | 6.0 Hz, 1H), 8.90 (d, J = 1.6 Hz, |
| | 756.29; | 1H), 8.22 (dd, J = 8.4, |
| | m/z found, | 2.4 Hz, 1H), 8.07 (d, |
| | 757.4 | J = 8.4 Hz, 1H), 7.96 (s, 1H), 7.92 |
| | [M + H]⁺. | (d, J = 0.8 Hz, 1H), 7.71 (s, |
| | | 1H), 7.66 (dd, J = 9.2, 1.2 Hz, |
| | | 1H), 7.57 (d, J = 8.4 Hz, |
| | | 1H), 7.36 (dd, J = 8.4, 1.2 Hz, |
| | | 1H), 7.07 (d, J = 1.6 Hz, 1H), |
| | | 6.99 (d, J = 1.6 Hz, 1H), |
| | | 6.51 (d, J = 9.2 Hz, 1H), |
| | | 4.35 (d, J = 6.0 Hz, 2H), 4.15 |
| | | (dd, J = 12.4, 4.8 Hz, 1H), |
| | | 3.80-3.75 (m, 2H), 3.70- |
| | | 3.64 (m, 1H), 3.60 (s, 3H), 3.31 |
| | | (s, 3H), 2.75-2.67 (m, |
| | | 1H), 2.63-2.55 (m, 3H), 2.36-2.29 |
| | | (m, 1H), 2.11-2.04 |
| | | (m, 1H), 1.28 (d, J = 6.8 Hz, 6H). |
| A143 | mass | ¹H NMR (400 MHz, DMSO-d₆) |
| | calcd. for | δ 10.88 (s, 1H), 9.37 (d, J = |
| | C₃₈H₃₂N₈O₆, | 1.4 Hz, 1H), 9.32 (t, J = 6.1 Hz, |
| | 696.2; m/z | 1H), 8.65 (dd, J = 8.2, |
| | found, 696.8 | 2.1 Hz, 1H), 8.15 (d, J = 8.2 |
| | [M + H]⁺. | Hz, 1H), 8.02 (s, 1H), 7.96 (s, |
| | | 1H), 7.71 (s, 1H), 7.58 (d, |
| | | J = 8.4 Hz, 1H), 7.38-7.35 (m, |
| | | 2H), 7.27-7.24 (m, 1H), |
| | | 7.14-7.11 (m, 1H), 4.65-4.62 |
| | | (m, 2H), 4.36 (d, |
| | | J = 5.9 Hz, 2H), 4.16-4.12 (m, 1H), |
| | | 3.88-3.86 (m, 2H), 3.84-3.81 |
| | | (m, 2H), 3.37 (s, 3H), |
| | | 2.72-2.65 (m, 1H), 2.60-2.56 |
| | | (m, 1H), 2.36-2.31 (m, |
| | | 1H), 2.11-2.06 (m, 1H), |
| | | 1.21-1.98 (m, 3H). |
| A144 | mass | ¹H NMR (400 MHz, DMSO-d₆) |
| | calcd. for | δ 10.88 (s, 1H), 9.37 (d, J = |
| | C₃₆H₂₇N₇O₇, | 2.1 Hz, 1H), 9.33 (s, 1H), 8.65 |
| | 669.2; m/z | (dd, J = 8.2, 2.1 Hz, 1H), |
| | found, 670.2 | 8.15 (d, J = 8.3 Hz, 1H), |
| | [M + H]⁺. | 8.05 (s, 1H), 7.96 (s, 1H), 7.71 |
| | | (d, J = 1.6 Hz, 1H), 7.58 (d, |
| | | J = 8.6 Hz, 1H), 7.52 (d, J = |
| | | 2.0 Hz, 1H), 7.39-7.37 (m, |
| | | 1H), 7.35 (d, J = 2.7 Hz, 1H), |
| | | 7.31 (d, J = 6.8 Hz, 1H), |
| | | 4.68-4.59 (m, 2H), 4.36 (d, J = |
| | | 5.9 Hz, 2H), 4.15 (m, 3H), |
| | | 3.87-3.70 (m, 3H), 2.75- |
| | | 2.65 (m, 1H), 2.63-2.55 (m, |
| | | 1H), 2.36-2.30 (m, 1H), |
| | | 2.14-1.98 (m, 1H). |
| A145 | mass | ¹H NMR (400 MHz, DMSO-d₆) |
| | calcd. for | δ 10.89 (s, 1H), 9.90 (s, |
| | C₄₃H₃₆N₈O₆, | 1H), 9.52-9.46 (m, 1H), 9.34 |
| | 760.3; m/z | (t, J = 5.9 Hz, 1H), 8.80 |
| | found, 761.2 | (dd, J = 8.2, 2.1 Hz, 1H), 8.74 |
| | [M + H]⁺. | (s, 1H), 8.23 (d, J = 8.2 Hz, |
| | | 1H), 8.12 (d, J = 8.0 Hz, 1H), |
| | | 7.96 (s, 1H), 7.94-7.86 (m, |
| | | 2H), 7.75-7.70 (m, 1H), 7.58 |
| | | (d, J = 8.6 Hz, 1H), 7.38 |
| | | (dd, J = 8.6, 1.4 Hz, 1H), 7.33 |
| | | (s, 1H), 4.39 (d, J = 5.9 Hz, |
| | | 2H), 4.16 (dd, J = 12.2, 4.8 Hz, |
| | | 1H), 4.06 (d, J = 8.1 Hz, |
| | | 2H), 3.96 (d, J = 8.2 Hz, 2H), |
| | | 3.56 (s, 3H), 3.40 (s, 3H), |
| | | 2.78-2.68 (m, 1H), 2.60-2.54 |
| | | (m, 1H), 2.40-2.28 (m, |
| | | 1H), 2.13-2.05 (m, 1H), 1.48 (s, 3H). |
| A146 | mass | ¹H NMR (400 MHz, DMSO-d₆) |
| | calcd. for | δ 10.88 (s, 1H), 9.41- |
| | C₃₉H₃₄N₈O₅, | 9.31 (m, 1H), 9.29 (s, 1H), 8.67 |
| | 694.3; m/z | (dd, J = 8.2, 2.1 Hz, 1H), |
| | found, 695.3 | 8.14 (d, J = 8.2 Hz, 1H), |
| | [M + H]⁺. | 7.96 (s, 1H), 7.91 (s, 1H), 7.71 |
| | | (d, J = 1.6 Hz, 1H), 7.58 (d, |
| | | J = 8.6 Hz, 1H), 7.37 (dd, J = |
| | | 8.5, 1.6 Hz, 1H), 7.09 (d, J = 2.0 |
| | | Hz, 1H), 6.86 (d, J = 1.9 |
| | | Hz, 1H), 4.36 (d, J = 5.9 Hz, |
| | | 2H), 4.15 (dd, J = 12.3, 4.9 |
| | | Hz, 1H), 3.72-3.65 (m, 2H), |
| | | 3.59 (s, 3H), 3.31 (s, 3H), |
| | | 3.10-2.99 (m, 2H), 2.86-2.65 |
| | | (m, 2H), 2.60 (s, 3H), |
| | | 2.39-2.26 (m, 1H), 2.22-2.12 |
| | | (m, 2H), 2.04-1.95 (m, |
| | | 1H). |
| A147 | mass | ¹H NMR (400 MHz, DMSO-d₆) |
| | calcd. for | δ 10.89 (s, 1H), 9.31 (s, |
| | C₄₀H₃₈N₁₀O₆, | 1H), 9.18 (t, J = 5.7 Hz, 1H), 8.93 |
| | 754.3; m/z | (s, 2H), 8.56 (d, 1H), |
| | found, 755.3 | 7.97 (s, 1H), 7.72 (d, 1H), |
| | [M + H]⁺. | 7.59 (d, J = 8.5 Hz, 1H), 7.41- |
| | | 7.35 (m, 1H), 6.81 (s, 1H), 4.34 (d, |
| | | J = 5.7 Hz, 2H), 4.19- |
| | | 4.13 (m, 1H), 3.75-3.71 (m, 4H), |
| | | 3.52 (s, 3H), 3.51 (s, |
| | | 3H), 3.32 (s, 3H), 3.01-2.96 |
| | | (m, 4H), 2.76-2.69 (m, |
| | | 1H), 2.61-2.57 (m, 1H), 2.40 |
| | | (s, 3H), 2.37-2.31 (m, |
| | | 1H), 2.13-2.05 (m, 1H). |
| A148 | mass | ¹H NMR (400 MHz, DMSO-d₆) |
| | calcd. for | δ 10.90 (s, 1H), 9.40 (s, |
| | C₄₁H₃₂N₆O₆, | 1H), 9.03 (d, J = 1.5 Hz, 1H), 8.95 |
| | 704.2; m/z | (m, 1H), 8.78 (s, 1H), |
| | found, 705.3 | 8.32 (d, J = 1.4 Hz, 1H), 8.10 |
| | [M + H]⁺. | (d, J = 8.2 Hz, 1H), 7.97 (s, |
| | | 1H), 7.92 (m, 1H), 7.73 (s, 1H), |
| | | 7.67 (d, J = 6.4 Hz, 1H), |
| | | 7.60 (d, 1H), 7.45 (s, 1H), |
| | | 7.40-7.34 (m, 2H), 7.32- |
| | | 7.29 (m, 1H), 4.33 |
| | | (d, J = 5.7 Hz, 2H), 4.17-4.13 (m, |
| | | 1H), 3.99 (s, 3H), 3.40 (s, 6H), |
| | | 2.74-2.67 (m, 1H), 2.60- |
| | | 2.55 (m, 1H), 2.37-2.31 (m, 1H), |
| | | 2.10-2.04 (m, 1H). |
| A149 | mass | ¹H NMR (400 MHz, DMSO-d₆) |
| | calcd. for | δ 10.88 (s, 1H), 9.08 (s, |
| | C₄₁H₃₉N₇O₆S, | 1H), 8.80 (d, J = 2.1 Hz, 1H), |
| | 757.3; m/z | 7.99 (d, J = 2.1 Hz, 1H), |
| | found, 758.3 | 7.96 (s, 1H), 7.71 (d, J = 1.6 |
| | [M + H]⁺. | Hz, 1H), 7.58 (d, J = 8.5 Hz, |
| | | 1H), 7.36 (dd, J = 8.5, 1.7 Hz, 1H), |
| | | 7.24 (s, 1H), 7.10 (d, J = |
| | | 2.1 Hz, 1H), 7.00 (d, J = 2.1 Hz, |
| | | 1H), 4.31 (d, J = 5.9 |
| | | Hz, 2H), 4.15 (dd, |
| | | J = 12.2, 4.9 Hz, 1H), 3.99-3.89 (m, |
| | | 2H), 3.60 (s, 3H), 3.56-3.46 (m, 2H), |
| | | 3.40 (s, 3H), 2.73- |
| | | 2.64 (m, 1H), 2.59 (s, 3H), |
| | | 2.49 (s, 3H), 2.37-2.22 (m, |
| | | 1H), 2.17-1.92 (m, 2H), |
| | | 1.80-1.68 (m, 5H). |
| A150 | mass | ¹H NMR (400 MHz, DMSO-d₆) |
| | calcd. for | δ 10.89 (s, 1H), 9.87 (s, |
| | C₄₄H₃₈N₈O₆, | 1H), 9.29 (s, 1H), 9.17 (t, |
| | 774.3; m/z | J = 5.8 Hz, 1H), 8.71 (s, 1H), |
| | found, 775.5 | 8.57 (s, 1H), 8.11 (d, |
| | [M + H]⁺. | J = 7.9 Hz, 1H), 7.97 (s, 1H), 7.95- |
| | | 7.86 (m, 2H), 7.73 (s, 1H), 7.59 (d, |
| | | J = 8.6 Hz, 1H), 7.54 |
| | | (s, 1H), 7.39 (dd, J = 8.5, 1.5 Hz, |
| | | 1H), 4.36 (d, J = 5.8 Hz, |
| | | 2H), 4.16 (dd, J = 12.2, 4.9 Hz, |
| | | 1H), 3.84-3.78 (m, 4H), |
| | | 3.64 (s, 3H), 3.42 (s, 3H), 3.17-3.11 |
| | | (m, 4H), 2.76-2.68 |
| | | (m, 4H), 2.60-2.54 (m, 1H), |
| | | 2.39-2.30 (m, 1H), 2.13- |
| | | 2.07 (m, 1H). |
| A151 | mass | ¹H NMR (400 MHz, DMSO-d₆) |
| | calcd. for | δ 10.82 (s, 1H), 8.55 (d, J = |
| | C₄₄H₄₀F₂N₆O₅, | 1.7 Hz, 1H), 8.26 (dd, J = 13.2, |
| | 770; m/z | 6.7 Hz, 2H), 7.82 (dd J = |
| | found, 771 | 8.0, 2.2 Hz, 1H), 7.61 |
| | [M + H]⁺. | (d, J = 6.4 Hz, 2H), 7.46-7.36 |
| | | (m, 2H), 7.01-6.96 (m, 2H), 6.87 |
| | | (s, 1H), 6.73 (d, J = 2.0 |
| | | Hz, 1H), 6.30 (t, J = 55.0 Hz, |
| | | 1H), 4.53 (d, J = 5.6 Hz, |
| | | 2H), 3.97 (t, J = 7.8 Hz, 1H), |
| | | 3.73-3.69 (m, 5H), 3.64 |
| | | (dd, J = 13.6, 6.8 Hz, 1H), |
| | | 3.39 (s, 3H), 2.98-2.94 (m, |
| | | 2H), 2.85-2.68 (m, 2H), 2.38-2.32 |
| | | (m, 2H), 2.18-2.12 |
| | | (m, 2H), 1.32 (d, J = 6.8 Hz, 6H). |
| A152 | mass | ¹H NMR (400 MHz, DMSO-d₆) |
| | calcd. for | δ 10.88 (s, 1H), 9.81 (s, |
| | C₄₂H₃₁N₉O₅, | 1H), 9.50 (d, J = 2.0 Hz, 1H), |
| | 741.2; m/z | 9.35 (t, J = 5.9 Hz, 1H), |
| | found, 742.2 | 8.84-8.76 (m, 2H), 8.38 (d, |
| | [M + H]⁺. | J = 2.4 Hz, 1H), 8.22 (d, J = |
| | | 8.2 Hz, 1H), 8.18 (dd, J = 6.1, |
| | | 3.3 Hz, 1H), 7.96 (dd, J = |
| | | 8.6, 5.2 Hz, 4H), 7.86 (s, |
| | | 1H), 7.72 (s, 1H), 7.58 (d, J = |
| | | 8.5 Hz, 1H), 7.38 (d, J = 8.5 Hz, |
| | | 1H), 6.61 (t, J = 2.0 Hz, |
| | | 1H), 4.38 (d, J = 5.9 Hz, 2H), |
| | | 4.18-4.14 (m, 1H), 3.52 (s, |
| | | 3H), 3.11 (s, 3H), 2.76-2.67 |
| | | (m, 1H), 2.60-2.54 (m, |
| | | 1H), 2.39-2.31 (m, 1H), |
| | | 2.13-2.04 (m, 1H). |
| A153 | mass | ¹H NMR (400 MHz, DMSO-d₆) |
| | calcd. for | δ 10.90 (s, 1H), 9.12 (t, J = |
| | C₄₁H₃₈FN₇O₅, | 5.9 Hz, 1H), 8.89 (s, 1H), |
| | 727.3; m/z | 8.16-8.12 (m, 1H), 7.99 (t, J = |
| | found, 728.7 | 2.1 Hz, 1H), 7.96 (s, 1H), |
| | [M + H]⁺. | 7.72 (d, J = 1.2 Hz, 1H), 7.60- |
| | | 7.56 (m, 1H), 7.39-7.34 (m, 1H), |
| | | 7.07-7.01 (m, 2H), |
| | | 6.98-6.94 (m, 1H), 4.34 (d, |
| | | J = 5.8 Hz, 2H), 4.19-4.13 |
| | | (m, 1H), 3.72-3.63 (m, 1H), |
| | | 3.60 (s, 3H), 3.38 (s, 3H), |
| | | 3.32 (s, 3H), 2.77-2.68 (m, 1H), |
| | | 2.64 (s, 3H), 2.61-2.54 |
| | | (m, 1H), 2.39-2.29 (m, 1H), |
| | | 2.12-2.07 (m, 1H), 1.29- |
| | | 1.25 (m, 6H). |
| A154 | mass | ¹H NMR (400 MHz, DMSO-d₆) |
| | calcd. for | δ 10.74 (s, 1H), 9.14 (s, |
| | C₃₇H₄₀N₁₀O₄, | 1H), 9.05 (s, 1H), 8.27 (s, 1H), |
| | 688.32; | 8.18 (s, 1H), 7.93 (d, J = |
| | m/z found, | 8.9 Hz, 1H), 7.17 (dd, |
| | 689.321 | J = 8.9, 3.0 Hz, 1H), 6.99 (d, J = |
| | [M + H]⁺. | 2.0 Hz, 1H), 6.92 (d, J = 2.0 Hz, |
| | | 1H), 5.75 (s, 1H), 5.64 |
| | | (d, J = 6.6 Hz, 1H), 4.34 (d, |
| | | J = 5.7 Hz, 2H), 4.17 (dd, J = |
| | | 12.2, 5.6 Hz, 1H), 3.63 (s, 3H), |
| | | 3.59 (s, 3H), 3.37 (s, 3H), |
| | | 3.30 (s, 3H), 2.70-2.65 (m, 1H), |
| | | 2.63 (s, 3H), 2.58-2.51 |
| | | (m, 2H), 2.18-2.14 (m, 1H), |
| | | 1.89-1.85 (m, 1H), 1.27 |
| | | (d, J = 6.8 Hz, 6H). |
| A155 | mass | ¹H NMR (400 MHz, DMSO-d₆) |
| | calcd. for | δ 10.89 (s, 1H), 9.39 (s, |
| | C₄₁H₃₂N₆O₅, | 1H), 9.30 (d, J = 2.1 Hz, 1H), |
| | 688.8; m/z | 9.18 (t, J = 5.9 Hz, 1H), |
| | found, 689.36 | 8.74 (s, 1H), 8.57 (d, J = 2.1 Hz, |
| | [M + H]⁺. | 1H), 8.09 (d, J = 8.3 Hz, |
| | | 1H), 7.97 (s, 1H), 7.91 (dd, J = 8.2, |
| | | 7.1 Hz, 1H), 7.73 (d, J = |
| | | 1.6 Hz, 1H), 7.67 (dd, J = 7.1, |
| | | 1.1 Hz, 1H), 7.59 (d, J = |
| | | 8.5 Hz, 1H), 7.45 (d, |
| | | J = 1.5 Hz, 1H), 7.40-7.13 (m, 3H), |
| | | 4.35 (d, J = 5.9 Hz, 2H), 4.16 |
| | | (dd, J = 12.2, 4.9 Hz, 1H), |
| | | 3.43 (s, 3H), 3.40 (s, 3H), 2.70 |
| | | (s, 3H), 2.58 (d, J = 4.0 |
| | | Hz, 1H), 2.34 (dd, J = 12.8, |
| | | 4.2 Hz, 1H), 2.19-2.05 (m, |
| | | 1H) |
| A156 | mass | ¹H NMR (400 MHz, DMSO-d₆) |
| | calcd. for | δ 10.89 (s, 1H), 9.23 (d, J = |
| | C₄₀H₃₈N₈O₅, | 1.7 Hz, 1H), 9.14 (t, J = 5.9 Hz, |
| | 710.3; m/z | 1H), 8.45 (d, J = 1.4 |
| | found, 711.3 | Hz, 1H), 8.37 (s, 2H), 7.96 (s, |
| | [M + H]⁺. | 1H), 7.72 (d, J = 1.2 Hz, |
| | | 1H), 7.58 (d, J = 8.5 Hz, 1H), |
| | | 7.37 (dd, J = 8.5, 1.5 Hz, |
| | | 1H), 7.06 (d, J = 2.0 Hz, 1H), |
| | | 6.98 (d, J = 2.0 Hz, 1H), |
| | | 4.33 (d, J = 5.8 Hz, 2H), 4.16 |
| | | (dd, J = 12.2, 4.9 Hz, 1H), |
| | | 3.66 (dt, J = 13.7, 6.9 Hz, 1H), 3.59 |
| | | (s, 3H), 3.40 (s, 3H), |
| | | 3.31 (s, 3H), 2.77-2.68 (m, 1H), |
| | | 2.65 (s, 3H), 2.61-2.54 |
| | | (m, 1H), 2.38-2.30 (m, 1H), |
| | | 2.13-2.05 (m, 1H), 1.27 |
| | | (d, J = 6.8 Hz, 6H). |
| A157 | mass | ¹H NMR (400 MHz, DMSO-d₆) |
| | calcd. for | δ 10.88 (s, 1H), 9.51 (d, J = |
| | C₃₉H₂₉N₇O₅, | 1.7 Hz, 1H), 9.38 (s, 1H), 9.35 |
| | 675.2; m/z | (t, J = 6.0 Hz, 1H), 8.82- |
| | found, 676.2 | 8.78 (m, 2H), 8.23 (d, |
| | [M + H]⁺. | J = 8.3 Hz, 1H), 8.20 (d, J = 1.7 |
| | | Hz, 1H), 8.15 (d, J = 8.3 Hz, 1H), |
| | | 7.96 (s, 1H), 7.94 (d, J = |
| | | 8.0 Hz, 1H), 7.84 (d, |
| | | J = 1.7 Hz, 1H), 7.73-7.69 (m, |
| | | 2H), 7.58 (d, J = 8.5 Hz, 1H), |
| | | 7.37 (dd, J = 8.6, 1.5 Hz, |
| | | 1H), 4.39 (d, J = 5.9 Hz, 2H), |
| | | 4.16 (dd, J = 12.2, 4.8 Hz, |
| | | 1H), 3.43 (d, J = 6.3 Hz, 6H), |
| | | 2.75-2.68 (m, 1H), 2.61- |
| | | 2.56 (m, 1H), 2.37-2.29 (m, 1H), |
| | | 2.11-2.04 (m, 1H). |
| A158 | mass | ¹H NMR (400 MHz, DMSO-d₆) |
| | calcd. for | δ 10.94 (s, 1H), 9.49 (d, J = |
| | C₄₃H₃₅FN₆O₅, | 2.0 Hz, 1H), 9.40 (s, 1H), 9.34 |
| | 734.27; | (t, J = 5.9 Hz, 1H), 8.86- |
| | m/z found, | 8.71 (m, 2H), 8.22 (d, |
| | 735.27 | J = 8.3 Hz, 1H), 8.10 (d, J = 8.0 |
| | [M + H]⁺. | Hz, 1H), 7.92 (t, J = 7.6 Hz, 1H), |
| | | 7.68 (d, J = 7.6 Hz, 2H), |
| | | 7.58 (d, J = 8.6 Hz, 1H), |
| | | 7.38 (dd, J = 8.6, 1.6 Hz, 1H), |
| | | 7.28 (s, 1H), 7.21 (s, 1H), 4.38 (d, |
| | | J = 5.9 Hz, 2H), 4.18- |
| | | 4.10 (m, 1H), 3.79-3.71 |
| | | (m, 1H), 3.66 (s, 3H), 3.39 (s, |
| | | 3H), 2.77-2.66 (m, 1H), |
| | | 2.59-2.54 (m, 1H), 2.41-2.31 |
| | | (m, 1H), 2.10-2.01 (m, 1H), |
| | | 1.35 (d, J = 6.8 Hz, 6H). |
| A159 | mass | ¹H NMR (400 MHz, DMSO-d₆) |
| | calcd. for | δ 10.91 (s, 1H), 9.50 (d, J = |
| | C₄₂H₃₅N₇O₅, | 1.7 Hz, 1H), 9.41 (d, J = 7.2 Hz, |
| | 717.2; m/z | 2H), 8.78 (d, J = 7.0 |
| | found, 717.6 | Hz, 2H), 8.30 (s, 1H), |
| | [M + H]⁺. | 8.22 (d, J = 8.2 Hz, 1H), 8.09 (s, |
| | | 1H), 8.04 (d, J = 8.6 Hz, 1H), |
| | | 7.92 (s, 1H), 7.69 (d, J = 6.9 |
| | | Hz, 1H), 7.49 (d, J = 8.6 Hz, |
| | | 1H), 7.28 (s, 1H), 7.21 (s, |
| | | 1H), 4.41 (d, J = 5.9 Hz, 2H), |
| | | 4.17 (dd, J = 12.4, 5.0 Hz, |
| | | 1H), 3.79-3.72 (m, 1H), 3.66 |
| | | (s, 3H), 3.39 (s, 3H), 2.80- |
| | | 2.72 (m, 1H), 2.61-2.55 |
| | | (m, 1H), 2.45-2.35 (m, 1H), |
| | | 2.17-2.08 (m, 1H), 1.35 |
| | | (d, J = 6.8 Hz, 6H). |
| A160 | mass | ¹H NMR (400 MHz, DMSO-d₆) |
| | calcd. for | δ 10.94 (s, 1H), 9.47 (t, J = |
| | C₃₇H₃₁N₉O₆, | 5.8 Hz, 1H), 9.37 (d, J = 1.8 |
| | 697.2; m/z | Hz, 1H), 8.71-8.62 (m, |
| | found, 698.2 | 1H), 8.44 (d, J = 5.8 Hz, |
| | [M + H]⁺. | 1H), 8.15 (d, J = 8.2 Hz, 1H), |
| | | 8.05 (s, 1H), 7.59 (d, J = 5.8 Hz, |
| | | 1H), 7.50 (s, 1H), 7.12 |
| | | (d, J = 1.7 Hz, 1H), 6.89 (d, |
| | | J = 1.3 Hz, 1H), 4.61 (t, J = |
| | | 4.2 Hz, 2H), 4.47-4.41 |
| | | (m, 3H), 3.80 (s, 2H), 3.59 (s, |
| | | 3H), 3.31 (s, 3H), 2.62-2.58 |
| | | (m, 4H), 2.57-2.53 (m, 1H), |
| | | 2.39-2.33 (m, 1H), 2.21-2.15 (m, 1H). |
| A161 | mass | ¹H NMR (400 MHz, DMSO-d₆) |
| | calcd. For | δ 10.82 (s, 1H), 9.29 (t, J = |
| | C₄₀H₃₇N₉O₅, | 5.9 Hz, 1H), 9.22 (d, J = 1.7 |
| | 723.29; m/z | Hz, 1H), 8.60 (d, J = 7.1 |
| | found, 724.29 | Hz, 1H), 8.51 (dd, |
| | [M + H]⁺. | J = 8.3, 2.1 Hz, 1H), 8.08 (d, J = 8.2 |
| | | Hz, 1H), 7.95 (d, J = 8.4 Hz, |
| | | 2H), 7.81 (s, 1H), 7.66 (s, |
| | | 1H), 7.07 (d, J = 1.8 Hz, 1H), |
| | | 7.01 (d, J = 1.9 Hz, 1H), |
| | | 6.80 (dd, J = 7.3, 1.6 Hz, |
| | | 1H), 4.47 (t, J = 6.3 Hz, 2H), |
| | | 4.38 (d, J = 5.8 Hz, 2H), 4.20 |
| | | (dd, J = 12.1, 4.8 Hz, 1H), |
| | | 3.81-3.78 (m, 2H), 3.69-3.64 |
| | | (m, 1H), 3.59 (s, 3H), |
| | | 3.32 (s, 3H), 2.72-2.65 (m, 1H), |
| | | 2.60-2.54 (m, 1H), |
| | | 2.36-2.27 (m, 1H), 2.11-2.03 |
| | | (m, 1H), 1.29 (d, J = 6.7 |
| | | Hz, 6H). |
| A162 | mass | ¹H NMR (400 MHz, DMSO-d₆) |
| | calcd. for | δ 10.88 (s, 1H), 9.52- |
| | C₃₉H₂₉N₇O₅, | 9.46 (m, 2H), 9.38 (s, 1H), |
| | 675.2; m/z | 8.83-8.79 (m, 2H), 8.24 (d, J = |
| | found, 676.2 | 8.4 Hz, 1H), 8.20 |
| | [M + H]⁺. | (d, J = 1.7 Hz, 1H), 8.15 (d, J = 8.4 |
| | | Hz, 1H), 7.98-7.93 (m, 1H), |
| | | 7.85 (d, J = 1.7 Hz, 1H), |
| | | 7.71 (d, J = 7.0 Hz, 1H), |
| | | 7.47 (d, J = 8.3 Hz, 1H), 7.37- |
| | | 7.32 (m, 1H), 7.30 (s, 1H), |
| | | 7.12 (d, J = 7.4 Hz, 1H), 4.45 |
| | | (d, J = 5.8 Hz, 2H), 4.21 |
| | | (dd, J = 12.1, 4.8 Hz, 1H), 3.44 |
| | | (s, 3H), 3.43 (s, 3H), 2.73-2.67 |
| | | (m, 1H), 2.57-2.53 (m, |
| | | 1H), 2.38-2.32 (m, 1H), |
| | | 2.08-2.01 (m, 1H). |
| A163 | mass | ¹H NMR (400 MHz, DMSO-d₆) |
| | calcd. for | δ 11.26 (s, 1H), 9.50 (d, J = |
| | C₃₈H₂₉N₉O₄, | 1.7 Hz, 1H), 9.43-9.36 |
| | 675.2; m/z | (m, 2H), 8.83-8.75 (m, 2H), |
| | found, 676.7 | 8.23 (d, J = 8.2 Hz, 1H), |
| | [M + H]⁺. | 8.13-8.06 (m, 2H), 8.03 (s, 1H), |
| | | 7.92-7.89 (m, 1H), 7.68 (d, |
| | | J = 7.1 Hz, 1H), 7.47-7.42 |
| | | (m, 2H), 7.37-7.33 (m, 1H), |
| | | 7.33-7.28 (m, 1H), 6.27- |
| | | 6.19 (m, 1H), 4.42 (d, |
| | | J = 5.9 Hz, 2H), 3.43 (s, 3H), 3.40 |
| | | (s, 3H), 3.00-2.91 (m, 1H), |
| | | 2.90-2.81 (m, 1H), 2.76- |
| | | 2.69 (m, 1H), 2.41-2.31 (m, 1H). |
| A164 | mass | ¹H NMR (400 MHz, DMSO-d₆) |
| | calcd. for | δ 10.87 (s, 1H), 9.50 (d, J = 2.1 Hz, |
| | C₄₃H₃₆N₆O₄S, | 1H), 9.42-9.32 (m, 2H), 8.84-8.74 (m, 2H), |
| | 732.3; m/z | 8.23 (d, J = 8.3 Hz, 1H), 8.10 |
| | found, 733.2 | (d, J = 8.4 Hz, 1H), 8.00 (d, |
| | [M + H]⁺. | J = 8.4 Hz, 1H), 7.95-7.87 (m, 2H), |
| | | 7.68 (d, J = 7.1 Hz, |
| | | 1H), 7.63 (s, 1H), 7.41 (dd, J = 8.4, |
| | | 1.4 Hz, 1H), 7.28 (d, J = |
| | | 1.5 Hz, 1H), 7.21 (d, |
| | | J = 1.5 Hz, 1H), 4.48-4.36 (m, |
| | | 3H), 3.81-3.71 (m, 1H), 3.66 |
| | | (s, 3H), 3.39 (s, 3H), 2.79- |
| | | 2.68 (m, 1H), 2.61-2.54 |
| | | (m, 1H), 2.42-2.30 (m, 1H), |
| | | 2.14-2.03 (m, 1H), 1.35 |
| | | (d, J = 6.8 Hz, 6H). |
| A165 | mass | ¹H NMR (400 MHz, DMSO-d₆) |
| | calcd. for | δ 10.88 (s, 1H), 9.47- |
| | C₃₇H₃₀N₈O₆, | 9.42 (m, 1H), 9.36 (d, J = 1.9 Hz, |
| | 682.7; m/z | 1H), 8.65 (dd, J = 8.2, |
| | found, 683.4 | 2.1 Hz, 1H), 8.15 (d, |
| | [M + H]⁺. | J = 8.3 Hz, 1H), 8.01 (s, 1H), 7.46 |
| | | (d, J = 8.3 Hz, 1H), 7.34 (t, |
| | | J = 7.9 Hz, 1H), 7.31-7.28 |
| | | (m, 2H), 7.24 (d, J = 8.3 Hz, 1H), |
| | | 7.12 (d, J = 7.7 Hz, 2H), |
| | | 4.66-4.60 (m, 2H), 4.42 (d, |
| | | J = 5.8 Hz, 2H), 4.21 (dd, J = |
| | | 12.1, 4.9 Hz, 1H), 3.82 (d, |
| | | J = 4.0 Hz, 2H), 3.37-3.35 |
| | | (m, 6H), 2.72-2.65 (m, 1H), |
| | | 2.58-2.54 (m, 1H), 2.38- |
| | | 2.29 (m, 1H), 2.05-1.98 (m, 1H). |
| A166 | mass | ¹H NMR (400 MHz, DMSO-d₆) |
| | calcd. for | δ 10.89 (s, 1H), 9.28 (s, |
| | C₄₄H₄₃N₇O₇, | 1H), 9.22 (s, 1H), 8.50 (s, 1H), |
| | 781.3; m/z | 8.38 (s, 1H), 7.83 (s, 1H), |
| | found, 782.3 | 7.48 (d, J = 8.0 Hz, 1H), 7.36 (d, |
| | [M + H]⁺. | J = 7.5 Hz, 1H), 7.31 (s, |
| | | 1H), 7.13 (d, J = 7.4 Hz, 1H), |
| | | 6.46 (s, 1H), 6.27 (s, 1H), |
| | | 4.43 (d, J = 3.8 Hz, 2H), |
| | | 4.25-4.21 (m, 1H), 3.94 (s, 3H), |
| | | 3.92-3.87 (m, 2H), |
| | | 3.53 (s, 3H), 3.51-3.47 (m, 3H), |
| | | 3.28 (s, 3H), 3.24 (s, 3H), |
| | | 2.72 (s, 3H), 2.71-2.67 (m, |
| | | 1H), 2.58-2.55 (m, 1H), |
| | | 2.37-2.31 (m, 1H), 2.15-2.06 |
| | | (m, 1H), 1.69-1.54 (m, 4H). |
| A167 | mass | ¹H NMR (400 MHz, DMSO-d₆) |
| | calcd. for | δ 10.89 (s, 1H), 9.37- |
| | C₄₃H₄₂N₈O₇, | 9.34 (m, 1H), 9.31 (s, 1H), |
| | 782.3; m/z | 8.61-8.58 (m, 1H), 8.47 (s, |
| | found, 783.3 | 1H), 7.48 (d, J = 8.3 Hz, 1H), |
| | [M + H]⁺. | 7.37-7.33 (m, 1H), 7.31 (s, |
| | | 1H), 7.13 (d, J = 7.4 Hz, 1H), |
| | | 6.64 (d, J = 2.1 Hz, 1H), |
| | | 6.41 (d, J = 2.1 Hz, 1H), |
| | | 4.42 (d, J = 5.9 Hz, 2H), 4.22 |
| | | (dd, J = 12.2, 4.8 Hz, 1H), |
| | | 4.00 (s, 3H), 3.92-3.87 (m, |
| | | 2H), 3.55 (s, 3H), 3.49-3.46 |
| | | (m, 2H), 3.43-3.42 (m, |
| | | 1H), 3.32 (s, 3H), 3.26 (s, 3H), |
| | | 2.76-2.70 (m, 1H), 2.69 |
| | | (s, 3H), 2.57-2.54 (m, 1H), |
| | | 2.40-2.33 (m, 1H), 2.02- |
| | | 1.96 (m, 1H), 1.68-1.61 (m, 4H). |
| A168 | mass | ¹H NMR (400 MHz, DMSO-d₆) |
| | calcd. for | δ 10.98 (s, 1H), 9.30 (t, J = |
| | C₄₁H₃₉N₉O₆, | 5.9 Hz, 1H), 9.24 (s, 1H), 8.49 |
| | 753.3; m/z | (d, J = 5.8 Hz, 1H), 8.46 |
| | found, 754.3 | (s, 1H), 8.38 (s, 2H), 7.66 (d, |
| | [M + H]⁺. | J = 5.7 Hz, 1H), 7.54 (s, |
| | | 1H), 7.10-7.08 (m, 1H), 7.02-6.98 |
| | | (m, 1H), 4.49 (dd, J = |
| | | 9.8, 5.1 Hz, 1H), 4.43 (d, |
| | | J = 5.8 Hz, 2H), 3.97-3.91 |
| | | (m, 2H), 3.62 (s, 3H), |
| | | 3.55-3.47 (m, 3H), 3.40 (s, 3H), |
| | | 3.31 (s, 3H), 2.66 (s, 3H), |
| | | 2.64-2.55 (m, 2H), 2.42-2.34 |
| | | (m, 1H), 2.23-2.15 (m, 1H), |
| | | 1.81-1.71 (m, 4H). |
| A169 | mass | ¹H NMR (400 MHz, DMSO-d₆) |
| | calcd. for | δ 10.88 (s, 1H), 9.62- |
| | C₄₀H₃₀N₆O₅, | 9.43 (m, 1H), 9.40 (s, 2H), |
| | 674.7; m/z | 8.85-8.66 (m, 1H), 8.23 (d, J = |
| | found, 675.7 | 8.2 Hz, 2H), 8.11 (d, |
| | [M + H]⁺. | J = 8.3 Hz, 1H), 8.01-7.82 (m, |
| | | 1H), 7.78-7.59 (m, 1H), 7.53-7.42 |
| | | (m, 2H), 7.35 (d, J = |
| | | 7.3 Hz, 2H), 7.33-7.27 (m, 2H), |
| | | 7.12 (d, J = 7.3 Hz, 1H), |
| | | 4.45 (d, J = 5.9 Hz, 2H), 4.21 |
| | | (dd, J = 12.1, 4.9 Hz, 1H), |
| | | 3.43 (s, 3H), 3.40 (s, 3H), |
| | | 2.78-2.60 (m, 1H), 2.58-2.53 |
| | | (m, 1H), 2.37-2.30 (m, 1H), |
| | | 2.05-1.97 (m, 1H). |
| A170 | mass | ¹H NMR (400 MHz, DMSO-d₆) |
| | calcd. for | δ 10.89 (s, 1H), 9.52- |
| | C₄₀H₃₀N₆O₅, | 9.45 (m, 2H), 9.40 (s, 1H), |
| | 674.2; m/z | 8.83-8.75 (m, 2H), 8.23 (d, J = |
| | found, 675.2 | 8.1 Hz, 1H), 8.11 (d, |
| | [M + H]⁺. | J = 8.1 Hz, 1H), 7.92 (t, J = 7.5 |
| | | Hz, 1H), 7.68 (d, J = 7.1 Hz, |
| | | 1H), 7.50-7.43 (m, 2H), |
| | | 7.37-7.32 (m, 2H), 7.32-7.28 |
| | | (m, 2H), 7.13 (d, J = 7.4 |
| | | Hz, 1H), 4.46 (d, |
| | | J = 5.6 Hz, 2H), 4.26-4.19 (m, 1H), |
| | | 3.44-3.42 (m, 6H), 2.74-2.66 |
| | | (m, 1H), 2.58-2.53 (m, 1H), |
| | | 2.39-2.30 (m, 1H), 2.08-2.01 (m, 1H). |
| A171 | mass | ¹H NMR (400 MHz, DMSO-d₆) |
| | calcd. for | δ 10.78 (s, 1H), 9.51- |
| | C₄₀H₃₀N₆O₅, | 9.45 (m, 2H), 9.40 (s, 1H), |
| | 674.23; m/z | 8.83-8.76 (m, 2H), 8.23 (d, J = |
| | found, 675.23 | 8.3 Hz, 1H), 8.12-8.08 |
| | [M + H]⁺. | (m, 1H), 7.92-7.82 (m, 1H), |
| | | 7.68 (d, J = 7.2 Hz, 1H), 7.49-7.44 |
| | | (m, 2H), 7.37-7.29 |
| | | (m, 4H), 7.13-7.10 (m, 1H), 4.45 |
| | | (d, J = 5.9 Hz, 2H), |
| | | 4.32-4.27 (m, 1H), 3.42 |
| | | (s, 3H), 3.05 (s, 3H), 2.87-2.78 |
| | | (m, 1H), 2.71-2.66 (m, 1H), |
| | | 2.36-2.31 (m, 1H), 2.04- |
| | | 1.98 (m, 1H). |
| A172 | mass | ¹H NMR (400 MHz, DMSO-d₆) |
| | calcd. for | δ 10.83 (s, 1H), 9.50 (d, J = |
| | C₄₂H₃₆N₈O₄, | 2.0 Hz, 1H), 9.41-9.38 (m, 2H), |
| | 716.29; m/z | 8.81 (dd, J = 8.2, 2.2 |
| | found, 717.29 | Hz, 1H), 8.77 (s, 1H), 8.62 (d, |
| | [M + H]⁺. | J = 7.2 Hz, 1H), 8.23 (d, J = |
| | | 8.2 Hz, 1H), 8.11 (d, J = 8.2 Hz, |
| | | 1H), 7.95 (s, 1H), 7.94- |
| | | 7.90 (m, 1H), 7.82 (s, 1H), 7.69 (d, |
| | | J = 7.1 Hz, 1H), 7.29 |
| | | (d, J = 1.4 Hz, 1H), 7.21 (d, |
| | | J = 1.4 Hz, 1H), 6.82 (dd, J = |
| | | 7.3, 1.8 Hz, 1H), 4.42 (d, J = 5.9 Hz, |
| | | 2H), 4.21 (dd, J = |
| | | 12.2, 4.8 Hz, 1H), 3.76-3.70 |
| | | (m, 1H), 3.66 (s, 3H), 3.39 |
| | | (s, 3H), 2.70-2.65 (m, 1H), |
| | | 2.60-2.54 (m, 1H), 2.34- |
| | | 2.29 (m, 1H), 2.11-2.05 (m, 1H), |
| | | 1.35 (d, J = 6.8 Hz, |
| | | 6H). |
| A173 | mass | ¹H NMR (400 MHz, DMSO-d₆) |
| | calcd. for | δ 10.88 (s, 1H), 9.27 (t, J = |
| | C₃₈H₃₂N₈O₆, | 6.0 Hz, 1H), 9.21-9.14 (m, |
| | 696.2; m/z | 1H), 8.45-8.37 (m, 1H), |
| | found, 697.2 | 8.00 (s, 1H), 7.47 (d, |
| | [M + H]⁺. | J = 8.3 Hz, 1H), 7.34 (t, J = 7.9 Hz, |
| | | 1H), 7.30 (s, 2H), 7.24 (d, |
| | | J = 8.3 Hz, 1H), 7.15-7.10 (m, |
| | | 2H), 4.65-4.59 (m, 2H), 4.39 (d, |
| | | J = 5.8 Hz, 2H), 4.25- |
| | | 4.19 (m, 1H), 3.84-3.78 (m, |
| | | 2H), 3.36 (s, 3H), 3.33 (s, |
| | | 3H), 2.74-2.67 (m, 1H), 2.65 |
| | | (s, 3H), 2.56-2.53 (m, |
| | | 1H), 2.37-2.31 (m, 1H), |
| | | 2.06-1.98 (m, 1H). |
| A174 | mass | ¹H NMR (400 MHz, DMSO-d₆) |
| | calcd. for | δ 10.88 (s, 1H), 9.27 (t, J = |
| | C₃₈H₃₂N₈O₆, | 6.1 Hz, 1H), 9.17 (s, 1H), |
| | 696.24; m/z | 8.39 (s, 1H), 8.00 (s, 1H), |
| | found, 697 | 7.47 (d, J = 8.3 Hz, 1H), |
| | [M + H]⁺. | 7.36-7.32 (m, 1H), 7.30 (s, 2H), |
| | | 7.24 (d, J = 8.4 Hz, 1H), |
| | | 7.14-7.10 (m, 2H), 4.64-4.60 |
| | | (m, 2H), 4.42-4.37 (m, 2H), |
| | | 4.22 (dd, J = 12.2, 4.9 Hz, |
| | | 1H), 3.83-3.78 (m, 2H), |
| | | 3.37-3.34 (m, 6H), 2.69-2.66 |
| | | (m, 1H), 2.65 (s, 3H), 2.56-2.53 |
| | | (m, 1H), 2.34-2.31 (m, |
| | | 1H), 2.07-2.00 (m, 1H). |
| A175 | mass | ¹H NMR (400 MHz, DMSO-d₆) |
| | calcd. for | δ 10.88 (s, 1H), 9.27 (t, J = |
| | C₃₈H₃₂N₈O₆, | 6.0 Hz, 1H), 9.17 (d, J = 1.6 Hz, |
| | 696.2; m/z | 1H), 8.39 (d, J = 1.4 |
| | found, 697.5 | Hz, 1H), 8.00 (s, 1H), 7.47 |
| | [M + H]⁺. | (d, J = 8.3 Hz, 1H), 7.37-7.32 |
| | | (m, 1H), 7.31-7.28 (m, 2H), |
| | | 7.24 (d, J = 8.3 Hz, 1H), |
| | | 7.14-7.10 (m, 2H), 4.65-4.59 |
| | | (m, 2H), 4.39 (d, J = 5.9 |
| | | Hz, 2H), 4.22 (dd, |
| | | J = 12.2, 4.8 Hz, 1H), |
| | | 3.84-3.78 (m, |
| | | 2H), 3.36 (s, 3H), 3.33 (s, 3H), |
| | | 2.73-2.66 (m, 1H), 2.65 |
| | | (s, 3H), 2.57-2.52 (m, 1H), |
| | | 2.38-2.31 (m, 1H), 2.07- |
| | | 2.01 (m, 1H). |
| A176 | mass | ¹H NMR (400 MHz, DMSO-d₆) |
| | calcd. for | δ 10.97 (s, 1H), 9.25 (t, J = |
| | C₄₁H₃₉N₉O₆, | 5.8 Hz, 1H), 9.04 (s, 1H), 8.92 |
| | 753.3; m/z | (s, 1H), 8.48 (d, J = 5.9 |
| | found, 753.8 | Hz, 1H), 8.28 (s, 1H), 7.92 |
| | [M + H]⁺. | (s, 1H), 7.65 (d, J = 5.8 Hz, |
| | | 1H), 7.53 (s, 1H), 7.16 (s, 1H), |
| | | 7.06 (d, J = 1.5 Hz, 1H), |
| | | 4.49-4.43 (m, 1H), |
| | | 4.42-4.38-4.35 (m, 2H), 3.96- |
| | | 3.94 (m, 2H), 3.93-3.90 |
| | | (m, 2H), 3.63 (s, 3H), 3.52- |
| | | 3.49 (m, 2H), 3.48 (s, 3H), 3.33 |
| | | (s, 3H), 2.65 (s, 3H), 2.59- |
| | | 2.52 (m, 1H), 2.40-2.34 |
| | | (m, 1H), 2.19-2.16 (m, 1H), |
| | | 1.78-1.71 (m, 4H). |
| A177 | mass | ¹H NMR (400 MHz, DMSO-d₆) |
| | calcd. for | δ 10.74 (s, 1H), 9.39 (s, |
| | C₄₃H₃₉N₇O₄, | 1H), 9.28 (d, J = 1.6 Hz, 1H), |
| | 717.3; m/z | 9.13 (t, J = 5.8 Hz, 1H), |
| | found, 718.3 | 8.72 (s, 1H), 8.55 (s, 1H), 8.08 |
| | [M + H]⁺. | (d, J = 8.2 Hz, 1H), 7.90 (t, |
| | | J = 7.7 Hz, 1H), 7.67 (d, J = 7.1 |
| | | Hz, 1H), 7.28 (s, 1H), |
| | | 7.21 (s, 1H), 7.04 (t, J = 8.2 Hz, |
| | | 1H), 6.65 (d, J = 7.5 Hz, |
| | | 1H), 6.47 (d, J = 6.5 Hz, 2H), |
| | | 6.43 (s, 1H), 4.31 (d, J = 5.8 |
| | | Hz, 2H), 3.79-3.71 (m, 1H), |
| | | 3.66 (s, 3H), 3.38 (s, 3H), |
| | | 2.97 (t, J = 6.3 Hz, 1H), 2.78-2.72 |
| | | (m, 2H), 2.69 (s, 3H), 2.50-2.45 |
| | | (m, 2H), 1.34 (d, J = 6.8 Hz, 6H). |
| A178 | mass | ¹H NMR (400 MHz, DMSO-d₆) |
| | calcd. for | δ 10.85 (s, 1H), 9.37- |
| | C₃₈H₃₀N₈O₆, | 9.34 (m, 1H), 9.32-9.27 |
| | 694.2; m/z | (m, 1H), 8.66-8.61 (m, 1H), |
| | found, 695.2 | 8.16-8.11 (m, 1H), 8.00 (s, |
| | [M + H]⁺. | 1H), 7.95 (s, 1H), 7.59-7.55 |
| | | (m, 2H), 7.38-7.34 (m, 1H), |
| | | 7.31-7.28 (m, 1H), 7.26- |
| | | 7.22 (m, 1H), 7.14-7.10 |
| | | (m, 1H), 4.65-4.60 (m, 2H), |
| | | 4.38-4.32 (m, 2H), |
| | | 3.84-3.78 (m, 2H), 3.36 (s, 3H), |
| | | 3.33 (s, 3H), 3.05-3.01 (m, 1H), |
| | | 2.92-2.87 (m, 2H), |
| | | 2.76-2.71 (m, 2H). |
| A179 | mass | ¹H NMR (400 MHz, DMSO-d₆) |
| | calcd. for | δ 10.88 (s, 1H), 9.55 (s, |
| | C₃₉H₂₉N₇O₅, | 1H), 9.53-9.51 (m, 1H), |
| | 675.2; m/z | 9.50-9.45 (m, 1H), 8.85-8.80 |
| | found, 676.4 | (m, 3H), 8.28-8.23 (m, 2H), |
| | [M + H]⁺. | 8.05-8.00 (m, 2H), 7.95- |
| | | 7.91 (m, 1H), 7.49-7.45 |
| | | (m, 1H), 7.37-7.32 (m, 1H), |
| | | 7.30 (s, 1H), 7.14-7.11 (m, 1H), 4.46 |
| | | (d, J = 5.8 Hz, 2H), |
| | | 4.23-4.20 (m, 1H), 3.52 |
| | | (d, J = 12.6 Hz, 6H), 2.72-2.66 |
| | | (m, 1H), 2.56-2.53 (m, 1H), |
| | | 2.39-2.30 (m, 1H), 2.07- |
| | | 2.00 (m, 1H). |
| A180 | | |
| A181 | mass | ¹H NMR (400 MHz, DMSO-d₆) |
| | calcd. for | δ 10.88 (s, 1H), 9.39 (s, |
| | C₄₁H₃₂N₆O₅, | 1H), 9.31 (d, J = 1.9 Hz, 2H), |
| | 688.24; m/z | 8.74 (s, 1H), 8.57 (s, 1H), |
| | found, 689.24 | 8.09 (d, J = 8.3 Hz, 1H), |
| | [M + H]⁺. | 7.95-7.86 (m, 1H), 7.67 (d, J = |
| | | 7.1 Hz, 1H), 7.48 (d, J = 8.3 Hz, |
| | | 1H), 7.45 (s, 1H), 7.35 (t, |
| | | J = 7.2 Hz, 2H), 7.30 (d, |
| | | J = 5.7 Hz, 2H), 7.13 (d, J = 7.4 |
| | | Hz, 1H), 4.43 (d, |
| | | J = 5.8 Hz, 2H), 4.26-4.19 (m, 1H), |
| | | 3.43 (s, 3H), 3.40 (s, 3H), |
| | | 2.71 (s, 3H), 2.67-2.61 (m, |
| | | 1H), 2.55-2.51 (m, 1H), |
| | | 2.33-2.30 (m, 1H), 2.07-2.01 |
| | | (m, 1H). |
| A182 | mass | ¹H NMR (400 MHz, DMSO-d₆) |
| | calcd. for | δ 10.87 (s, 1H), 9.38 (s, |
| | C₄₁H₃₂N₆O₅, | 2H), 8.67 (dd, J = 8.2, 2.1 Hz, |
| | 688.2; m/z | 2H), 8.11 (d, J = 8.2 Hz, |
| | found, 689.2 | 1H), 7.81 (s, 1H), 7.69 |
| | [M + H]⁺. | (t, J = 8.2 Hz, 1H), 7.52 (d, J = 8.4 |
| | | Hz, 1H), 7.46 (d, J = 8.3 Hz, |
| | | 1H), 7.31 (dd, J = 14.6, 6.8 |
| | | Hz, 4H), 7.12 (d, J = 7.3 Hz, |
| | | 1H), 6.87 (s, 1H), 4.42 (d, J = |
| | | 6.0 Hz, 2H), |
| | | 4.23-4.19 (m, 1H), 4.00-3.91 (m, 2H), |
| | | 3.71 (s, 3H), 3.66-3.61 (m, 2H), |
| | | 3.37 (s, 3H), 2.73-2.67 |
| | | (m, 2H), 2.34-2.31 (m, 1H), |
| | | 2.11 (s, 3H), 2.06-2.00 (m, |
| | | 1H). |
| A183 | mass | ¹H NMR (400 MHz, DMSO-d₆) |
| | calcd. for | δ 10.88 (s, 1H), 9.48 (dd, |
| | C₄₁H₃₂N₆O₅, | J = 12.7, 6.9 Hz, 2H), 9.41 |
| | 688.24; m/z | (s, 1H), 8.84-8.74 (m, 2H), |
| | found, 689 | 8.23 (d, J = 8.3 Hz, 1H), |
| | [M + H]⁺ | 8.09 (d, J = 8.3 Hz, 1H), 7.91 (t, |
| | | J = 7.7 Hz, 1H), 7.66 (d, |
| | | J = 7.1 Hz, 1H), 7.47 (d, J = 8.4 |
| | | Hz, 1H), 7.37-7.27 (m, 3H), |
| | | 7.12 (d, J = 7.3 Hz, 1H), |
| | . | 7.07 (s, 1H), 4.49-4.41 (m, |
| | | 2H), 4.21 (dd, J = 12.2, 4.8 |
| | | Hz, 1H), 3.66 (s, 3H), 3.38 (s, 3H), |
| | | 2.70 (s, 3H), 2.68- |
| | | 2.64 (m, 1H), 2.54 (d, |
| | | J = 4.1 Hz, 1H), 2.36-2.31 (m, |
| | | 1H), 2.07-2.00 (m, 1H). |
| A184 | mass | ¹H NMR (400 MHz, DMSO-d₆) |
| | C₄₃H₃₉N₇O₄, | δ 10.79 (s, 1H), 9.51- |
| | 717.3; m/z | 9.46 (m, 1H), 9.40 (s, 1H), |
| | found, 718.3 | 9.30 (s, 1H), 8.83-8.75 (m, |
| | [M + H]⁺. | 2H), 8.22 (d, J = 8.2 Hz, 1H), |
| | | 8.11 (d, J = 8.3 Hz, 1H), |
| | | 7.95-7.89 (m, 1H), 7.69 (dd, |
| | | J = 7.2, 1.1 Hz, 1H), 7.29 |
| | | (d, J = 1.6 Hz, 1H), 7.22 (d, |
| | | J = 1.6 Hz, 1H), 6.99 (d, J = |
| | | 7.5 Hz, 1H), 6.63 (dd, |
| | | J = 7.4, 1.3 Hz, 1H), 6.58-6.51 |
| | | (m, 1H), 4.71 (dd, J = 13.0, 4.8 Hz, |
| | | 1H), 4.34 (d, J = 6.0 |
| | | Hz, 2H), 3.76-3.75 (m, 1H), |
| | | 3.67 (s, 3H), 3.47-3.42 (m, |
| | | 1H), 3.39 (s, 3H), 3.31-3.24 |
| | | (m, 1H), 2.96-2.92 (m, |
| | | 1H), 2.80-2.68 (m, 1H), |
| | | 2.60-2.53 (m, 1H), 2.23-2.13 |
| | | (m, 1H), 2.04-1.98 (m, 1H), |
| | | 1.95-1.88 (m, 1H), 1.35 |
| | | (d, J = 6.8 Hz, 6H). |
| A185 | mass | ¹H NMR (400 MHz, DMSO-d₆) |
| | calcd. for | δ 11.01 (s, 1H), 9.61- |
| | C₄₃H₃₅FN₆O₅, | 9.53 (m, 2H), 9.48 (s, 1H), 8.89 |
| | 734.2; m/z | (dd, J = 8.2, 2.2 Hz, 1H), |
| | found, 735.3 | 8.86 (s, 1H), 8.31 (d, |
| | [M + H]⁺. | J = 8.2 Hz, 1H), 8.21-8.15 (m, 1H), |
| | | 8.04-7.96 (m, 1H), |
| | | 7.80-7.73 (m, 1H), 7.50 (s, 1H), |
| | | 7.39-7.34 (m, 2H), 7.31-7.27 (m, |
| | | 1H), 7.23-7.16 (m, |
| | | 1H), 4.55 (d, |
| | | J = 5.9 Hz, 2H), |
| | | 4.36 (dd, J = 12.8, 4.8 Hz, |
| | | 1H), 3.88-3.79 (m, 1H), 3.74 |
| | | (s, 3H), 3.47 (s, 3H), 2.93- |
| | | 2.83 (m, 1H), 2.67-2.62 (m, |
| | | 1H), 2.48-2.41 (m, 1H), |
| | | 2.15-2.08 (m, 1H), 1.43 |
| | | (d, J = 6.8 Hz, 6H). |
| A186 | mass | ¹H NMR (400 MHz, DMSO-d₆) |
| | calcd. for | δ 10.84 (s, 1H), 9.48 (s, |
| | C₄₃H₃₈N₈O₄, | 1H), 9.40 (s, 2H), 8.78 (d, |
| | 730.3; m/z | J = 10.4 Hz, 2H), 8.45 (s, 2H), |
| | found, 731.5 | 8.21 (d, J = 8.2 Hz, 1H), 8.10 |
| | [M + H]⁺. | (d, J = 7.6 Hz, 1H), 7.91 (d, |
| | | J = 7.6 Hz, 1H), 7.68 (s, 1H), |
| | | 7.56 (s, 1H), 7.39 (s, 1H), |
| | | 7.28 (s, 1H), 4.69-4.66 (m, |
| | | 1H), 4.38 (s, 2H), 3.86 (s, |
| | | 3H), 3.76 (s, 1H), 3.66 |
| | | (s, 3H), 3.26-3.24 (m, 3H), 2.79- |
| | | 2.78 (m, 1H), 2.69-2.62 (m, 1H), |
| | | 2.36-2.32 (m, 1H), |
| | | 1.98-1.95 (m, 1H), 1.35 |
| | | (d, J = 6.4 Hz, 6H). |
| A187 | mass | ¹H NMR (400 MHz, DMSO-d₆) |
| | calcd. for | δ 10.89 (s, 1H), 9.41 (t, J = |
| | C₄₁H₃₉N₉O₆, | 5.8 Hz, 1H), 9.28 (s, 1H), 8.41 |
| | 753.3; m/z | (d, J = 1.2 Hz, 2H), 7.48 |
| | found, 754.3 | (d, J = 8.3 Hz, 1H), 7.35 (t, |
| | [M + H]⁺. | J = 7.9 Hz, 1H), 7.30 (s, 1H), |
| | | 7.15-7.10 (m, 2H), 7.02 (s, 1H), |
| | | 4.43 (d, J = 5.6 Hz, 2H), |
| | | 4.25-4.19 (m, 1H), 3.96-3.91 |
| | | (m, 2H), 3.62 (s, 3H), |
| | | 3.56-3.53 (m, 1H), 3.51-3.48 |
| | | (m, 2H), 3.46-3.44 (m, |
| | | 3H), 3.32 (s, 3H), 2.85 (s, 3H), |
| | | 2.74-2.67 (m, 1H), 2.58- |
| | | 2.53 (m, 1H), 2.38-2.31 (m, 1H), |
| | | 2.07-2.01 (m, 1H), |
| | | 1.80-1.72 (m, 4H). |
| A188 | mass | ¹H NMR (400 MHz, DMSO-d₆) |
| | calcd. for | δ 10.88 (s, 1H), 9.45- |
| | C₃₉H₃₄N₈O₅, | 9.36 (m, 2H), 8.67 (dd, J = 8.2, |
| | 694.3; m/z | 2.1 Hz, 1H), 8.14 (d, J = |
| | found, 695.3 | 8.3 Hz, 1H), 7.91 (s, 1H), |
| | [M + H]⁺. | 7.49-7.43 (m, 1H), 7.37-7.31 |
| | | (m, 1H), 7.29 (s, 1H), 7.12 (d, |
| | | J = 7.4 Hz, 1H), 7.08 (d, J = |
| | | 1.8 Hz, 1H), 6.87-6.83 (m, 1H), |
| | | 4.42 (d, J = 5.9 Hz, |
| | | 2H), 4.24-4.18 (m, 1H), |
| | | 3.69-3.64 (m, 2H), 3.59 (s, |
| | | 3H), 3.30 (s, 3H), 3.07-3.01 |
| | | (m, 2H), 2.73-2.66 (m, |
| | | 1H), 2.59 (s, 3H), 2.56-2.53 |
| | | (m, 1H), 2.38-2.30 (m, |
| | | 1H), 2.20-2.12 (m, 2H), |
| | | 2.05-1.99 (m, 1H). |
| A189 | mass | ¹H NMR (400 MHz, DMSO-d₆) |
| | calcd. for | δ 10.89 (s, 1H), 9.37 (s, |
| | C₄₀H₃₁N₇O₅, | 1H), 9.31 (s, 2H), 8.76 (s, 1H), |
| | 689; m/z | 8.57 (s, 1H), 8.19 (s, 1H), |
| | found, 690 | 8.14 (d, J = 8.2 Hz, 1H), 7.94 |
| | | (t, J = 7.7 Hz, 1H), 7.84 (s, |
| | | 1H), 7.70 (d, J = 7.0 Hz, 1H), |
| | | 7.48 (d, J = 8.3 Hz, 1H), |
| | | 7.34 (dd, J = 16.6, 8.3 Hz, 2H), 7.13 |
| | | (d, J = 7.3 Hz, 1H), |
| | | 4.43 (d, J = 3.8 Hz, 2H), 4.23 |
| | | (dd, J = 12.2 Hz, 1H), 3.48- |
| | | 3.44 (m, 6H), 2.76-2.70 (m, 4H), |
| | | 2.60-2.55 (m, 1H), |
| | | 2.40-2.35 (m, 1H), 2.15-2.06 (m, 1H). |
| A190 | mass | ¹H NMR (400 MHz, DMSO-d₆) |
| | calcd. for | δ 11.22 (s, 1H), 9.43- |
| | C₃₅H₂₉N₁₁O₅, | 9.33 (m, 2H), 8.64 (dd, J = 8.2, 2.1 |
| | 683.2; m/z | Hz, 1H), 8.15 (d, J = |
| | [M + H]⁺. | 8.3 Hz, 1H), 8.07 (d, |
| | | J = 8.7 Hz, 1H), 8.02-7.99 (m, 2H), |
| | | 7.43 (dd, J = 8.6, 1.1 Hz, 1H), |
| | | 7.30 (d, J = 1.8 Hz, 1H), |
| | | 7.26-7.23 (m, 1H), 7.14-7.10 |
| | | (m, 1H), 6.26-6.20 (m, |
| | | 1H), 4.67-4.60 (m, 2H), 4.39 |
| | | (d, J = 5.9 Hz, 2H), 3.84- |
| | | 3.79 (m, 2H), 3.36 (s, 3H), |
| | | 3.34-3.33 (m, 3H), 3.03- |
| | | 2.92 (m, 1H), 2.91-2.82 (m, 1H), |
| | | 2.77-2.69 (m, 1H), |
| | | 2.41-2.34 (m, 1H). |
| A191 | mass | ¹H NMR (400 MHz, DMSO-d₆) |
| | calcd. for | δ 10.94 (s, 1H), 9.51 (t, J = |
| | C₄₃H₃₅FN₆O₅, | 4.3 Hz, 2H), 9.40 (s, 1H), |
| | 734.3; m/z | 8.83-8.75 (m, 2H), 8.23 (d, J = |
| | found, 735.3 | 8.3 Hz, 1H), 8.10 (d, |
| | [M + H]⁺. | J = 8.3 Hz, 1H), 7.96-7.88 (m, |
| | | 1H), 7.69 (d, J = 7.1 Hz, 1H), |
| | | 7.63 (dd, J = 7.0, 2.0 Hz, |
| | | 1H), 7.41-7.35 (m, 2H), 7.29 |
| | | (d, J = 1.4 Hz, 1H), 7.21 |
| | | (d, J = 1.4 Hz, 1H), 4.51 (d, J = 5.9 |
| | | Hz, 2H), 4.31 (dd, J = |
| | | 12.7, 5.0 Hz, 1H), 3.80-3.72 |
| | | (m, 1H), 3.66 (s, 3H), 3.39 |
| | | (s, 3H), 2.86-2.73 (m, 1H), |
| | | 2.60-2.54 (m, 1H), 2.42- |
| | | 2.32 (m, 1H), 2.07-1.99 |
| | | (m, 1H), 1.35 (s, 3H), 1.34 (s, |
| | | 3H). |
| A192 | mass | ¹H NMR (400 MHz, DMSO-d₆) |
| | calcd. for | δ 10.95 (s, 1H), 9.30 (t, J = |
| | C₃₆H₃₁N₉O₅, | 5.9 Hz, 1H), 9.25-9.20 (m, 1H), |
| | 669.2; m/z | 8.48-8.43 (m, 2H), |
| | found, 670.2 | 8.37 (s, 2H), 7.62 (d, |
| | [M + H]⁺. | J = 5.7 Hz, 1H), 7.52 (s, 1H), 7.25- |
| | | 7.21 (m, 2H), 7.05 (dd, |
| | | J = 8.3, 1.8 Hz, 1H), 4.48-4.45 |
| | | (m, 1H), 4.42 (d, J = 5.8 Hz, |
| | | 2H), 3.40 (s, 3H), 3.36 (s, |
| | | 3H), 3.33 (s, 3H), 2.66 (s, |
| | | 3H), 2.62-2.58 (m, 1H), 2.57- |
| | | 2.52 (m, 1H), 2.41-2.33 |
| | | (m, 1H), 2.23-2.15 (m, 1H). |
| A193 | mass | ¹H NMR (400 MHz, DMSO-d₆) |
| | calcd. for | δ 10.89 (s, 1H), 9.77 (s, |
| | C₄₁H₃₈N₈O₆, | 1H), 9.25 (t, J = 6.0 Hz, 1H), |
| | 738.3; m/z | 9.10 (d, J = 2.0 Hz, 1H), |
| | found, 739.3 | 8.94 (s, 1H), 8.56-8.16 (m, 2H), |
| | [M + H]⁺. | 7.59 (d, J = 1.9 Hz, 1H), |
| | | 7.48 (d, J = 8.3 Hz, 1H), 7.35 (t, |
| | | J = 7.9 Hz, 1H), 7.32- |
| | | 7.24 (m, 2H), 7.13 (d, J = 7.4 Hz, 1H), |
| | | 4.40 (d, J = 5.9 Hz, |
| | | 2H), 4.22 (dd, J = 12.1, |
| | | 4.8 Hz, 1H), 3.99-3.95 (m, 2H), |
| | | 3.58 (s, 3H), 3.55-3.48 (m, 2H), |
| | | 3.32 (s, 3H), 2.74-2.70 |
| | | (m, 1H), 2.67 (s, 3H), 2.58-2.55 |
| | | (m, 1H), 2.37-2.31 (m, |
| | | 1H), 2.15-2.06 (m, 2H), |
| | | 1.79-1.64 (m, 4H). |
| A194 | mass | ¹H NMR (400 MHz, DMSO-d₆) |
| | calcd. for | δ 10.85 (s, 1H), 9.87 (s, |
| | C₄₆H₃₈N₈O₆, | 1H), 9.51-9.47 (m, 1H), |
| | 798.3; m/z | 9.35-9.30 (m, 1H), 8.84-8.79 |
| | found, 799.3 | (m, 1H), 8.75 (s, 1H), |
| | [M + H]⁺. | 8.24-8.21 (m, 1H), 8.15-8.11 (m, |
| | | 1H), 7.95 (s, 1H), 7.95-7.91 |
| | | (m, 1H), 7.90-7.87 (m, |
| | | 1H), 7.60-7.56 (m, 3H), |
| | | 7.40-7.36 (m, 1H), 4.41-4.36 |
| | | (m, 4H), 3.71 (s, 3H), 3.42 (s, |
| | | 3H), 3.26-3.23 (m, 2H), |
| | | 3.08-3.04 (m, 2H), 3.04-3.01 |
| | | (m, 1H), 2.92-2.88 (m, |
| | | 2H), 2.77-2.72 (m, 2H), 2.14-2.09 |
| | | (m, 2H), 1.92-1.86 |
| | | (m, 2H). |
| A195 | mass | ¹H NMR (400 MHz, DMSO-d₆) |
| | calcd. for | δ 10.93 (s, 1H), 9.30 (t, J = |
| | C₃₇H₃₁N₉O₆, | 5.9 Hz, 1H), 9.18-9.16 (m, 1H), |
| | 697.2; m/z | 8.43 (d, J = 5.8 Hz, |
| | found, 698.4 | 1H), 8.40-8.39 (m, 1H), 8.00 |
| | [M + H]⁺. | (s, 1H), 7.57 (d, J = 5.8 Hz, |
| | | 1H), 7.49 (s, 1H), 7.30 (d, J = 1.6 Hz, |
| | | 1H), 7.24 (d, J = 8.3 |
| | | Hz, 1H), 7.12 (dd, J = 8.3, |
| | | 1.7 Hz, 1H), 4.64-4.61 (m, |
| | | 2H), 4.46-4.41 (m, 3H), |
| | | 3.83-3.79 (m, 2H), 3.36 (s, |
| | | 3H), 3.33 (s, 3H), 2.65 (s, 3H), |
| | | 2.62-2.56 (m, 2H), 2.39- |
| | | 2.33 (m, 1H), 2.21-2.15 (m, 1H). |
| A196 | mass | ¹H NMR (400 MHz, DMSO-d₆) |
| | calcd. for | δ 10.90 (s, 1H), 9.50 (d, J = |
| | C₄₂H₃₅N₇O₅, | 1.8 Hz, 1H), 9.42-9.35 |
| | 717.27; m/z | (m, 2H), 8.82-8.76 (m, 2H), |
| | found, 718 | 8.38 (d, J = 2.0 Hz, 1H), |
| | [M + H]⁺. | 8.26-8.21 (m, 2H), 8.12-8.08 |
| | | (m, 2H), 7.95-7.89 (m, 1H), |
| | | 7.69 (d, J = 7.1 Hz, 1H), |
| | | 7.28 (d, J = 1.5 Hz, 1H), 7.21 (d, |
| | | J = 1.4 Hz, 1H), 4.46- |
| | | 4.38 (m, 2H), 4.15 (dd, |
| | | J = 12.5, 5.0 Hz, 1H), 3.80-3.71 |
| | | (m, 1H), 3.66 (s, 3H), 3.39 |
| | | (s, 3H), 2.74-2.66 (m, 1H), |
| | | 2.60-2.54 (m, 1H), 2.42-2.32 |
| | | (m, 1H), 2.12-2.05 (m, |
| | | 1H), 1.35 (d, J = 6.8 Hz, 6H). |
| A197 | mass | ¹H NMR (400 MHz, DMSO-d₆) |
| | calcd. for | δ 10.89 (s, 1H), 9.48 (dd, |
| | C₄₃H₃₆N₆O₅, | J = 12.4, 6.8 Hz, 2H), 9.40 |
| | 716.27; m/z | (s, 1H), 8.84-8.75 (m, 2H), |
| | found, 717.4 | 8.23 (d, J = 8.4 Hz, 1H), 8.11 |
| | [M + H]⁺. | (d, J = 8.4 Hz, 1H), 7.92 (t, |
| | | J = 7.6 Hz, 1H), 7.69 (d, |
| | | J = 7.2 Hz, 1H), 7.48 (d, J = 8.4 |
| | | Hz, 1H), 7.35 (t, J = 8.0 Hz, 1H), |
| | | 7.30 (d, J = 7.6 Hz, 2H), |
| | | 7.22 (s, 1H), 7.13 (d, |
| | | J = 7.6 Hz, 1H), 4.46 (d, J = 5.6 Hz, |
| | | 2H), 4.22 (dd, J = 12.0, 4.8 Hz, |
| | | 1H), 3.75 (dd, J = 13.6, |
| | | 6.8 Hz, 1H), 3.66 (s, 3H), |
| | | 3.39 (s, 3H), 2.74-2.67 (m, |
| | | 1H), 2.59-2.53 (m, 1H), |
| | | 2.31-2.39 (m, 1H), 2.09-2.01 |
| | | (m, 1H), 1.35 (d, J = 6.8 Hz, 6H). |
| A198 | mass | ¹H NMR (400 MHz, DMSO-d₆) |
| | calcd. for | δ 10.89 (s, 1H), 9.29- |
| | C₄₅H₄₆N₈O₆, | 9.23 (m, 1H), 9.14-9.11 (m, |
| | 794.4; m/z | 1H), 8.42-8.40 (m, 1H), |
| | found, 795.4 | 8.15 (s, 1H), 7.49-7.46 (m, 1H), |
| | [M + H]⁺. | 7.46 (s, 1H), 7.37-7.33 |
| | | (m, 1H), 7.30 (s, 1H), 7.14-7.12 |
| | | (m, 1H), 6.42-6.39 (m, |
| | | 1H), 6.23-6.20 (m, 1H), |
| | | 4.44-4.39 (m, 2H), 4.24-4.19 |
| | | (m, 1H), 3.92-3.86 (m, 2H), |
| | | 3.53 (s, 3H), 3.50-3.44 (m, |
| | | 2H), 3.42-3.38 (m, 1H), 3.25 |
| | | (s, 3H), 3.19 (s, 3H), 2.95 |
| | | (s, 6H), 2.79-2.70 (m, 1H), |
| | | 2.69 (s, 3H), 2.56-2.52 (m, |
| | | 1H), 2.37-2.32 (m, 1H), 2.07-2.02 |
| | | (m, 1H), 1.70-1.57 |
| | | (m, 4H). |
| A199 | mass | ¹H NMR (400 MHz, DMSO-d₆) |
| | calcd. for | δ 10.88 (s, 1H), 9.45 (t, J = |
| | C₃₈H₃₂N₈O₆, | 5.9 Hz, 1H), 9.36 (d, J = 1.6 Hz, |
| | 696; m/z | 1H), 8.65 (dd, J = 8.2, |
| | found, 697 | 2.1 Hz, 1H), 8.15 (d, |
| | [M + H]⁺. | J = 8.2 Hz, 1H), 8.05 (s, 1H), 7.46 |
| | | (d, J = 8.3 Hz, 1H), 7.38-7.31 |
| | | (m, 1H), 7.29 (s, 1H), 7.15- |
| | | 7.08 (m, 2H), 6.89 (s, 1H), 4.61 |
| | | (d, J = 3.9 Hz, 2H), 4.42 |
| | | (d, J = 5.9 Hz, 2H), 4.21 (dd, |
| | | J = 12.2, 4.9 Hz, 1H), 3.79 |
| | | (s, 2H), 3.59 (s, 3H), 3.31 |
| | | (s, 3H), 2.74-2.66 (m, 1H), |
| | | 2.60 (s, 3H), 2.56-2.53 (m, 1H), |
| | | 2.38-2.30 (m, 1H), |
| | | 2.06-2.00 (m, 1H). |
| A200 | mass | ¹H NMR (400 MHz, DMSO-d₆) |
| | calcd. for | δ 10.99 (s, 1H), 9.38 (t, J = |
| | C₄₀H₃₈N₁₀O₆, | 5.9 Hz, 1H), 9.10 (s, 1H), 9.07 |
| | 754.8; m/z | (d, J = 1.4 Hz, 1H), 8.50 |
| | found, 764.8 | (d, J = 5.9 Hz, 1H), 7.90 |
| | [M + H]⁺. | (d, J = 1.4 Hz, 1H), 7.68 (d, J = |
| | | 5.9 Hz, 1H), 7.55 (s, 1H), 7.19 |
| | | (d, J = 2.0 Hz, 1H), 7.09 |
| | | (d, J = 2.1 Hz, 1H), 4.51 (d, |
| | | J = 5.8 Hz, 2H), 4.44 (dd, J = |
| | | 10.0, 5.2 Hz, 1H), |
| | | 3.94 (s, 3H), 3.63 (s, 3H), 3.55-3.43 |
| | | (m, 4H), 3.33 (s, 3H), 2.86 |
| | | (s, 3H), 2.61-2.51 (m, 1H), |
| | | 2.43-2.33 (m, 2H), 2.22-2.13 |
| | | (m, 2H), 1.77-1.42 (m, |
| | | 4H). |
| A201 | mass | ¹H NMR (400 MHz, DMSO-d₆) |
| | calcd. for | δ 10.85 (s, 1H), 9.23 (s, |
| | C₄₃H₄₀N₈O₆, | 1H), 9.13 (t, J = 5.8 Hz, 1H), |
| | 764.31; m/z | 8.45 (s, 1H), 8.38 (s, 2H), |
| | found, 765 | 7.96 (s, 1H), 7.60-7.56 (m, 2H), |
| | [M + H]⁺. | 7.37 (d, J = 9.8 Hz, 1H), |
| | | 7.09 (d, J = 1.8 Hz, 1H), 6.99 |
| | | (d, J = 1.9 Hz, 1H), 4.34- |
| | | 4.30 (m, 2H), 3.96-3.91 |
| | | (m, 2H), 3.62 (s, 3H), 3.55- |
| | | 3.49 (m, 3H), 3.41 (s, 3H), 3.31 |
| | | (s, 3H), 3.05-3.01 (m, |
| | | 1H), 2.92-2.87 (m, 2H), |
| | | 2.78-2.72 (m, 2H), 2.65 (s, |
| | | 3H), 1.78-1.71 (m, 4H). |
| A202 | mass | ¹H NMR (400 MHz, DMSO-d₆) |
| | calcd. for | δ 10.80 (s, 1H), 9.55- |
| | C₄₃H₃₈N₈O₄, | 9.43 (m, 1H), 9.40 (s, 1H), 9.35 |
| | 730.3; m/z | (t, J = 5.9 Hz, 1H), 8.82- |
| | found, 731.3 | 8.78 (m, 1H), 8.77 (s, 1H), 8.32 |
| | [M + H]⁺. | (d, J = 1.8 Hz, 1H), 8.23 |
| | | (d, J = 8.3 Hz, 1H), 8.10 (d, |
| | | J = 8.3 Hz, 1H), 8.06 (d, J = |
| | | 1.9 Hz, 1H), 7.91 (t, J = 7.7 Hz, |
| | | 1H), 7.68 (d, J = 6.9 Hz, |
| | | 1H), 7.47 (s, 1H), |
| | | 7.30-7.26 (m, 1H), 7.22-7.20 (m, |
| | | 1H), 4.41 (d, J = 5.9 Hz, 2H), |
| | | 4.18-4.07 (m, 1H), 3.79 (s, |
| | | 3H), 3.77-3.70 (m, 1H), 3.66 |
| | | (s, 3H), 3.40-3.38 (m, |
| | | 3H), 2.74-2.64 (m, 1H), 2.59-2.52 |
| | | (m, 1H), 2.37-2.27 |
| | | (m, 1H), 2.12-2.05 (m, 1H), 1.34 |
| | | (d, J = 6.8 Hz, 6H). |
| A203 | mass | ¹H NMR (400 MHz, DMSO-d₆) |
| | calcd. for | δ 10.89 (s, 1H), 9.30 (t, J = |
| | C₃₈H₃₁FN₈O₆, | 5.9 Hz, 1H), 9.17 (s, 1H), |
| | 714.2; m/z | 8.40 (s, 1H), 8.00 (s, 1H), |
| | found, 715.2 | 7.42 (d, J = 2.7 Hz, 1H), |
| | [M + H]⁺. | 7.30 (s, 1H), 7.28-7.23 (m, 2H), |
| | | 7.13 (s, 1H), 7.11 (s, 1H), 4.62 (t, |
| | | J = 4.4 Hz, 2H), 4.41 (d, |
| | | J = 5.8 Hz, 2H), 4.24-4.19 |
| | | (m, 1H), 3.82-3.80 (m, 2H), |
| | | 3.36 (s, 3H), 3.33 (s, 3H), |
| | | 2.74-2.67 (m, 1H), 2.65 (s, |
| | | 3H), 2.59-2.54 (m, 1H), |
| | | 2.39-2.32 (m, 1H), 2.05-1.99 |
| | | (m, 1H). |
| A204 | mass | ¹H NMR (400 MHz, DMSO-d₆) |
| | calcd. for | δ 13.07 (s, 1H), 10.88 (s, |
| | C₄₂H₃₆N₈O₄, | 1H), 9.49 (s, 1H), 9.40 (s, 1H), |
| | 716.2; m/z | 9.36-9.29 (m, 1H), 8.83- |
| | found, 717.2 | 8.74 (m, 2H), 8.23 (d, |
| | [M + H]⁺. | J = 8.2 Hz, 1H), 8.13-8.06 (m, |
| | | 1H), 7.94-7.87 (m, 2H), 7.68 |
| | | (d, J = 6.3 Hz, 1H), 7.53- |
| | | 7.47 (m, 1H), 7.39-7.33 |
| | | (m, 1H), 7.28 (d, J = 1.5 Hz, |
| | | 1H), 7.21 (d, J = 1.4 Hz, |
| | | 1H), 4.43-4.35 (m, 3H), 3.79- |
| | | 3.72 (m, 1H), 3.66 (s, 3H), 3.39 |
| | | (s, 3H), 2.72-2.56 (m, |
| | | 2H), 2.41-2.34 (m, 1H), 2.19-2.12 |
| | | (m, 1H), 1.35 (d, J = |
| | | 6.8 Hz, 6H). |
| A205 | mass | ¹H NMR (400 MHz, DMSO-d₆) |
| | calcd. for | δ 10.88 (s, 1H), 9.90 (s, |
| | C₄₂H₃₄N₈O₅, | 1H), 9.55-9.39 (m, 2H), 8.81 |
| | 730.3; m/z | (dd, J = 8.2, 2.1 Hz, 1H), |
| | found, 730.8 | 8.75 (s, 1H), 8.23 (d, |
| | [M + H]⁺. | J = 8.2 Hz, 1H), 8.12 (d, J = 7.9 Hz, |
| | | 1H), 7.90 (dt, J = 14.3, 7.2 Hz, |
| | | 2H), 7.47 (d, J = 8.3 Hz, |
| | | 1H), 7.38-7.29 (m, 3H), 7.12 |
| | | (d, J = 7.4 Hz, 1H), 4.46- |
| | | 4.40 (m, 2H), 4.22-4.18 |
| | | (m, 1H), 4.15-4.10 (m, 4H), |
| | | 3.56 (s, 3H), 3.39 (s, 3H), |
| | | 2.72-2.67 (m, 1H), 2.54-2.50 |
| | | (m, 1H), 2.37-2.28 (m, 3H), |
| | | 2.09-2.00 (m, 1H). |
| A206 | mass | ¹H NMR (400 MHz, DMSO-d₆) |
| | calcd. for | δ 10.99 (s, 1H), 9.89 (s, |
| | C₄₂H₃₅N₉O₆, | 1H), 9.52-9.46 (m, 2H), 8.81 (dd, |
| | 761.3; m/z | J = 8.2, 2.1 Hz, 1H), |
| | found, 762.3 | 8.76 (s, 1H), 8.51 (d, |
| | [M + H]⁺. | J = 6.0 Hz, 1H), 8.24 (d, J = 8.3 Hz, |
| | | 1H), 8.13 (d, J = 7.7 Hz, 1H), |
| | | 7.96-7.89 (m, 2H), 7.69 |
| | | (d, J = 5.8 Hz, 1H), 7.56 (s, 2H), |
| | | 4.52-4.46 (m, 3H), 3.84- |
| | | 3.79 (m, 4H), 3.64 |
| | | (s, 3H), 3.42 (s, 3H), 3.17-3.14 (m, |
| | | 4H), 2.68-2.56 (m, 2H), |
| | | 2.43-2.35 (m, 1H), 2.22-2.15 |
| | | (m, 1H). |
| A207 | mass | ¹H NMR (400 MHz, DMSO-d₆) |
| | calcd. for | δ 10.88 (s, 1H), 9.27 (d, J = |
| | C₄₂H₄₀N₈O₇, | 6.6 Hz, 2H), 8.51 (s, 1H), |
| | 768.30; m/z | 8.45 (s, 1H), 7.92 (s, 1H), |
| | found, 769 | 7.47 (d, J = 8.3 Hz, 1H), 7.35 |
| | [M + H]⁺. | (t, J = 8.0 Hz, 1H), 7.30 (s, |
| | | 1H), 7.12 (d, J = 7.5 Hz, 1H), |
| | | 6.93 (d, J = 6.5 Hz, 2H), |
| | | 4.41 (d, J = 5.8 Hz, 2H), 4.22 (dd, |
| | | J = 12.0, 4.8 Hz, 1H), |
| | | 4.17 (s, 3H), 3.98-3.92 |
| | | (m, 2H), 3.58 (s, 3H), 3.55-3.46 |
| | | (m, 3H), 3.29 (s, 3H), 2.75-2.66 |
| | | (m, 4H), 2.56-2.52 (m, |
| | | 1H), 2.36-2.32 (m, 1H), |
| | | 2.06-2.00 (m, 1H), 1.78-1.72 |
| | | (m, 4H). |
| A208 | mass | ¹H NMR (400 MHz, DMSO-d₆) |
| | calcd. for | δ 10.94 (s, 1H), 9.49 (s, |
| | C₄₂H₃₅N₇O₄S, | 1H), 9.46 (t, J = 5.9 Hz, 1H), |
| | 733.2; m/z | 9.40 (s, 1H), 8.80 (dd, J = |
| | found, 734.2 | 8.2, 2.0 Hz, 1H), 8.76 (s, 1H), |
| | [M + H]⁺. | 8.41 (d, J = 5.7 Hz, 1H), |
| | | 8.23 (d, J = 8.2 Hz, 1H), |
| | | 8.10 (d, J = 8.3 Hz, 1H), 8.00- |
| | | 7.97 (m, 2H), 7.92 (t, 1H), 7.69 |
| | | (d, J = 7.0 Hz, 1H), 7.29- |
| | | 7.26 (m, 1H), 7.22-7.19 |
| | | (m, 1H), 4.68 (dd, J = 9.5, 5.2 |
| | | Hz, 1H), 4.47 (d, J = 5.8 Hz, 2H), |
| | | 3.77-3.73 (m, 1H), |
| | | 3.66 (s, 3H), 3.41 (s, 3H), 2.62-2.55 |
| | | (m, 2H), 2.42-2.35 |
| | | (m, 1H), 2.21-2.15 (m, 1H), |
| | | 1.34 (d, J = 6.8 Hz, 6H). |
| A209 | mass | ¹H NMR (400 MHz, DMSO-d₆) |
| | calcd. for | δ 10.94 (s, 1H), 9.52 (t, J = |
| | C₃₉H₃₅N₉O₅, | 5.9 Hz, 1H), 9.10 (d, |
| | 709.3; m/z | J = 1.8 Hz, 1H), 8.50-8.41 (m, |
| | found, 709.7 | 2H), 8.26 (d, J = 8.2 Hz, 1H), |
| | [M + H]⁺. | 7.58 (d, J = 5.9 Hz, 1H), |
| | | 7.50 (s, 1H), 7.32 (s, 1H), 7.18 |
| | | (s, 1H), 7.09 (d, J = 1.8 |
| | | Hz, 1H), 6.86 (d, J = 1.4 |
| | | Hz, 1H), 4.50-4.40 (m, 3H), |
| | | 3.80 (d, J = 4.2 Hz, 2H), |
| | | 3.74 (d, J = 4.4 Hz, 4H), 3.60 (s, |
| | | 4H), 3.35 (s, 3H), 2.62-2.54 |
| | | (m, 5H), 2.37-2.31 (m, |
| | | 1H), 2.18-2.10 (m, 1H). |
| A210 | mass | ¹H NMR (400 MHz, DMSO-d₆) |
| | calcd. for | δ 10.88 (s, 1H), 9.89 (s, |
| | C₄₄H₃₆N₈O₆, | 1H), 9.47 (dd, J = 12.4, 6.7 Hz, |
| | 772.3; m/z | 2H), 8.86-8.71 (m, 2H), |
| | found, 772.6 | 8.23 (d, J = 8.2 Hz, 1H), |
| | [M + H]⁺. | 8.13 (d, J = 7.7 Hz, 1H), 7.97- |
| | | 7.86 (m, 2H), 7.55 (s, 1H), 7.47 (d, |
| | | J = 8.2 Hz, 1H), 7.33 |
| | | (dd, J = 15.5, 7.7 Hz, 2H), 7.12 |
| | | (d, J = 7.4 Hz, 1H), 4.61 |
| | | (d, J = 6.0 Hz, 2H), 4.46 |
| | | (d, J = 5.9 Hz, 2H), 4.21 (m, 1H), |
| | | 3.73-3.68 (m, 2H), 3.61 |
| | | (s, 3H), 3.54-3.48 (m, 2H), |
| | | 3.43 (s, 3H), 3.12-3.06 |
| | | (m, 1H), 2.74-2.66 (m, 1H), |
| | | 2.55 (s, 1H), 2.35-2.30 |
| | | (m, 2H), 2.03-1.98 (m, 1H). |
| A211 | mass | ¹H NMR (400 MHz, DMSO-d₆) |
| | calcd. for | δ 11.08 (s, 1H), 9.43 (d, J = |
| | C₃₈H₃₆N₁₀O₄, | 2.1 Hz, 1H), 9.35 (t, J = 6.0 Hz, |
| | 696.4; m/z | 1H), 8.70 (dd, J = 8.2, |
| | found, 697.4 | 2.2 Hz, 1H), 8.38 (s, 2H), 8.16 |
| | [M + H]⁺. | (d, J = 8.5 Hz, 2H), 7.86- |
| | | 7.59 (m, 2H), 7.17 (dd, J = 8.3, |
| | | 1.2 Hz, 1H), 7.06 (d, J = |
| | | 2.0 Hz, 1H), 6.98 (d, J = 2.1 Hz, |
| | | 1H), 5.89 (d, J = 11.3 Hz, |
| | | 1H), 4.38 (d, J = 5.9 |
| | | Hz, 2H), 3.74-3.60 (m, 1H), 3.59 (s, |
| | | 3H), 3.41 (s, 3H), 3.31 (s, 3H), |
| | | 2.88-2.62 (m, 2H), 2.35- |
| | | 2.20 (m, 2H), 1.27 (d, J = 6.8 Hz, 6H). |
| A212 | mass | ¹H NMR (400 MHz, DMSO-d₆) |
| | calcd. for | δ 10.85 (s, 1H), 9.55- |
| | C₄₄H₃₆N₆O₅, | 9.46 (m, 1H), 9.40 (s, 1H), 9.33 |
| | 728.3; m/z | (t, J = 5.9 Hz, 1H), 8.83- |
| | found, 729.3 | 8.73 (m, 2H), 8.22 (d, |
| | [M + H]⁺. | J = 8.2 Hz, 1H), 8.10 (d, J = 8.2 Hz, |
| | | 1H), 7.95 (s, 1H), 7.91 (t, J = 7.7 |
| | | Hz, 1H), 7.68 (d, J = 7.1 |
| | | Hz, 1H), 7.61-7.56 (m, 2H), |
| | | 7.42-7.36 (m, 1H), 7.28 (s, |
| | | 1H), 7.21 (s, 1H), 4.39 |
| | | (d, J = 5.8 Hz, 2H), 3.80-3.72 (m, |
| | | 1H), 3.66 (s, 3H), 3.40-3.38 |
| | | (m, 3H), 3.06-3.01 (m, |
| | | 1H), 2.93-2.87 (m, 2H), 2.78-2.71 |
| | | (m, 2H), 1.37-1.32 |
| | | (m, 6H). |
| A213 | mass | ¹H NMR (400 MHz, DMSO-d₆) |
| | calcd. for | δ 10.99 (s, 1H), 9.52- |
| | C₃₉H₂₉N₇O₅, | 9.49 (m, 2H), 9.40 (s, 1H), 8.80 |
| | 675.2; m/z | (dd, J = 8.2, 2.2 Hz, 1H), |
| | found, 676.3 | 8.77 (s, 1H), 8.50 (d, J = 5.9 |
| | [M + H]⁺. | Hz, 1H), 8.25-8.22 (m, 1H), |
| | | 8.10 (d, J = 8.4 Hz, 1H), |
| | | 7.94-7.90 (m, 1H), 7.70-7.67 |
| | | (m, 2H), 7.57-7.55 (m, 1H), |
| | | 7.44 (d, J = 1.3 Hz, 1H), |
| | | 7.35 (d, J = 8.0 Hz, 1H), 7.30 |
| | | (dd, J = 8.0, 1.5 Hz, 1H), |
| | | 4.52-4.47 (m, 3H), 3.43 (s, 3H), |
| | | 3.40 (s, 3H), 2.66-2.55 (m, 2H), |
| | | 2.42-2.34 (m, 1H), 2.23-2.16 (m, 1H). |
| A214 | mass | ¹H NMR (400 MHz, DMSO-d₆) |
| | calcd. for | δ 11.01 (s, 1H), 9.50 (s, |
| | C₄₃H₃₆N₈O₄, | 1H), 9.45-9.39 (m, 2H), 9.25 |
| | 728.3; m/z | (s, 1H), 8.81 (dd, J = 8.2, |
| | found, 729.3 | 2.1 Hz, 1H), 8.77 (s, 1H), 8.50 |
| | [M + H]⁺. | (s, 1H), 8.23 (d, J = 8.2 Hz, |
| | | 1H), 8.10 (d, J = 8.1 Hz, 1H), |
| | | 8.02 (s, 2H), 7.94-7.90 (m, |
| | | 1H), 7.69 (d, J = 6.9 Hz, 1H), |
| | | 7.28 (s, 1H), 7.21 (s, 1H), |
| | | 5.21 (dd, J = 10.5, 5.0 Hz, 1H), |
| | | 4.47 (d, J = 5.7 Hz, 2H), |
| | | 3.80-3.73 (m, 1H), 3.66 (s, 3H), |
| | | 3.39 (s, 3H), 2.73-2.67 |
| | | (m, 1H), 2.67-2.60 (m, 2H), |
| | | 2.23-2.18 (m, 1H), 1.37- |
| | | 1.32 (m, 6H). |
| A215 | mass | ¹H NMR (400 MHz, DMSO-d₆) |
| | calcd. for | δ 10.84 (s, 1H), 9.38 (d, J = |
| | C₃₉H₃₂N₈O₅, | 2.1 Hz, 1H), 9.26 (t, |
| | 692.2; m/z | J = 6.0 Hz, 1H), 8.70-8.62 (m, |
| | found, 693.2 | 1H), 8.13 (d, J = 8.2 Hz, |
| | [M + H]⁺. | 1H), 7.95 (s, 1H), 7.88 (s, 1H), |
| | | 7.60-7.55 (m, 2H), 7.36 (dd, |
| | | J = 8.7, 1.5 Hz, 1H), 7.24 |
| | | (d, J = 8.1 Hz, 2H), 7.08 (dd, |
| | | J = 8.2, 2.0 Hz, 1H), 4.35 (d, |
| | | J = 5.9 Hz, 2H), 3.71-3.65 |
| | | (m, 2H), 3.36 (s, 3H), 3.33 (s, |
| | | 3H), 3.09-3.01 (m, 3H), |
| | | 2.91-2.86 (m, 2H), 2.76-2.70 |
| | | (m, 2H), 2.20-2.12 (m, 2H). |
| A216 | mass | ¹H NMR (400 MHz, DMSO-d₆) |
| | calcd. for | δ 10.91 (s, 1H), 9.53- |
| | C₄₃H₃₆N₆O₅, | 9.43 (m, 2H), 9.40 (s, 1H), 8.80 |
| | 716.2; m/z | (dd, J = 8.2, 2.2 Hz, 1H), |
| | found, 717.3 | 8.77 (s, 1H), 8.22 (d, |
| | [M + H]⁺. | J = 8.3 Hz, 1H), 8.15-8.06 (m, 1H), |
| | | 7.95-7.88 (m, 1H), 7.71-7.66 |
| | | (m, 1H), 7.60-7.53 (m, |
| | | 1H), 7.30-7.27 (m, 2H), |
| | | 7.27-7.22 (m, 2H), 7.22-7.19 |
| | | (m, 1H), 4.46 (d, J = 5.9 Hz, 2H), |
| | | 4.27 (dd, J = 12.6, 5.0 |
| | | Hz, 1H), 3.80-3.71 (m, 1H), |
| | | 3.66 (s, 3H), 3.39 (s, 3H), |
| | | 2.85-2.75 (m, 1H), 2.60-2.53 |
| | | (m, 1H), 2.43-2.31 (m, |
| | | 1H), 2.09-2.01 (m, 1H), |
| | | 1.35 (d, J = 6.8 Hz, 6H). |
| A217 | mass | ¹H NMR (400 MHz, DMSO-d₆) |
| | calcd. for | δ 10.88 (s, 1H), 9.44 (t, J = |
| | C₃₈H₃₂N₈O₅, | 6.0 Hz, 1H), 9.26 (d, J = 2.1 Hz, |
| | 680.2; m/z | 1H), 8.53 (dd, J = 8.3, |
| | found, 681.2 | 2.2 Hz, 1H), 8.15 (d, |
| | [M + H]⁺. | J = 8.3 Hz, 1H), 8.00 (s, 1H), 7.46 |
| | | (d, J = 8.3 Hz, 1H), 7.34 (t, |
| | | J = 7.9 Hz, 1H), 7.29 (s, 1H), |
| | | 7.25-7.19 (m, 2H), 7.19-7.07 |
| | | (m, 2H), 4.42 (d, J = 5.9 |
| | | Hz, 2H), 4.21 (dd, |
| | | J = 12.2, 4.9 Hz, 1H), 4.05-3.70 (m, |
| | | 2H), 3.37 (s, 3H), 3.33 (s, 3H), |
| | | 3.03-2.92 (m, 2H), 2.77- |
| | | 2.62 (m, 1H), 2.58-2.52 (m, |
| | | 1H), 2.42-2.27 (m, 1H), |
| | | 2.18-1.90 (m, 3H). |
| A218 | mass | ¹H NMR (400 MHz, DMSO-d₆) |
| | calcd. for | δ 10.95 (s, 1H), 9.52 (t, J = |
| | C₃₈H₃₃N₉O₅, | 5.9 Hz, 1H), 9.10 (d, |
| | 695.3; m/z | J = 1.7 Hz, 1H), 8.48-8.44 (m, |
| | found, 695.8 | 2H), 8.27 (d, J = 8.2 Hz, 1H), |
| | [M + H]⁺. | 7.60 (d, J = 5.9 Hz, 1H), |
| | | 7.51 (s, 1H), 7.32 (s, 1H), 7.29 (d, |
| | | J = 8.3 Hz, 2H), 7.12 |
| | | (s, 1H), 7.10-7.08 (m, 1H), |
| | | 4.50-4.44 (m, 3H), 3.84- |
| | | 3.81 (m, 2H), 3.78-3.75 |
| | | (m, 2H), 3.38 (s, 3H), 3.36 (s, |
| | | 3H), 3.35 (s, 3H), 2.63-2.57 |
| | | (m, 2H), 2.39-2.33 (m, |
| | | 1H), 2.21-2.16 (m, 1H). |
| A219 | mass | ¹H NMR (400 MHz, DMSO-d₆) |
| | calcd. for | δ 10.88 (s, 1H), 9.43- |
| | C₃₉H₃₅N₉O₅, | 9.35 (m, 2H), 8.69 (dd, J = 8.2, |
| | 709.5; m/z | 2.1 Hz, 1H), 8.12 (d, J = |
| | found, 710.2 | 8.1 Hz, 1H), 7.49 (s, 1H), |
| | [M + H]⁺. | 7.46 (d, J = 8.3 Hz, 1H), 7.36- |
| | | 7.32 (m, 1H), 7.29 (s, 1H), 7.12 |
| | | (d, J = 7.3 Hz, 1H), 7.02 |
| | | (d, J = 1.9 Hz, 1H), 6.80 (d, |
| | | J = 1.4 Hz, 1H), 4.43 (d, J = |
| | | 5.9 Hz, 2H), 4.23-4.19 (m, |
| | | 1H), 3.68 (d, J = 3.4 Hz, 4H), |
| | | 3.58 (s, 3H), 3.30 (s, 3H), |
| | | 3.28 (s, 3H), 2.74-2.66 (m, |
| | | 1H), 2.59 (s, 3H), 2.56-2.52 |
| | | (m, 1H), 2.38-2.30 (m, |
| | | 1H), 2.06-2.00 (m, 1H). |
| A220 | mass | ¹H NMR (400 MHz, DMSO-d₆) |
| | calcd. for | δ 10.95 (s, 1H), 9.47 (t, J = |
| | C₃₈H₃₄N₁₀O₅, | 5.9 Hz, 1H), 9.38 (d, J = 1.5 Hz, |
| | 710.3; m/z | 1H), 8.69 (dd, J = 8.2, |
| | found, 711.5 | 2.1 Hz, 1H), 8.45 (d, |
| | [M + H]⁺. | J = 5.8 Hz, 1H), 8.18 (d, J = 8.1 Hz, |
| | | 1H), 7.60 (d, J = 5.9 Hz, |
| | | 1H), 7.51 (s, 1H), 7.39 (s, 1H), |
| | | 7.05 (d, J = 1.8 Hz, 1H), |
| | | 6.83 (s, 1H), 4.49-4.42 (m, 3H), |
| | | 3.76-3.73 (m, 4H), |
| | | 3.59 (s, 3H), 3.36 (s, 3H), 3.31 (s, |
| | | 3H), 2.68-2.62 (m, 1H), 2.59 |
| | | (s, 3H), 2.56-2.53 (m, |
| | | 1H), 2.38-2.33 (m, 1H), |
| | | 2.21-2.15 (m, 1H). |
| A221 | mass | ¹H NMR (400 MHz, DMSO-d₆) |
| | calcd. for | δ 10.92 (s, 1H), 9.50 (d, J = |
| | C₄₂H₃₇N₉O₄, | 1.8 Hz, 1H), 9.41-9.35 (m, 2H), |
| | 731.30; m/z | 8.80 (dd, J = 8.2, 2.2 |
| | found, 732.4 | Hz, 1H), 8.77 (s, 1H), 8.60 (d, |
| | [M + H]⁺. | J = 1.9 Hz, 1H), 8.40 (d, J = |
| | | 1.9 Hz, 1H), 8.23 (d, J = 8.3 |
| | | Hz, 1H), 8.10 (d, J = 8.3 |
| | | Hz, 1H), 7.96-7.89 (m, 1H), |
| | | 7.69 (d, J = 7.1 Hz, 1H), |
| | | 7.28 (d, J = 1.5 Hz, 1H), 7.21 |
| | | (d, J = 1.4 Hz, 1H), 4.45- |
| | | 4.37 (m, 3H), 4.02 (s, 3H), |
| | | 3.79-3.71 (m, 1H), 3.66 (s, |
| | | 3H), 3.39 (s, 3H), 2.70-2.64 |
| | | (m, 1H), 2.62-2.56 (m, |
| | | 1H), 2.46-2.39 (m, 1H), 2.18-2.11 |
| | | (m, 1H), 1.35 (d, J = |
| | | 6.8 Hz, 6H). |
| A222 | mass | ¹H NMR (400 MHz, DMSO-d₆) |
| | calcd. for | δ 10.88 (s, 1H), 9.38- |
| | C₄₁H₃₉N₉O₆, | 9.16 (m, 2H), 8.77 (s, 1H), |
| | 753; m/z | 8.65 (s, 2H), 8.49 (d, J = 1.4 |
| | found, 754 | Hz, 1H), 7.48 (d, J = 8.3 Hz, |
| | [M + H]⁺. | 1H), 7.34 (dd, J = 17.6, 9.3 |
| | | Hz, 2H), 7.13 (d, J = 7.3 Hz, |
| | | 1H), 6.96 (d, J = 1.9 Hz, |
| | | 1H), 6.83 (d, J = 1.9 Hz, 1H), |
| | | 4.41 (d, J = 5.9 Hz, 2H), |
| | | 4.22 (dd, J = 12.2, 4.9 Hz, 1H), |
| | | 3.96 (d, J = 11.0 Hz, 2H), |
| | | 3.59 (s, 3H), 3.54-3.47 (m, 3H), |
| | | 3.32 (s, 3H), 2.80-2.69 |
| | | (m, 1H), 2.67 (s, 3H), 2.58-2.54 |
| | | (m, 1H), 2.38-2.32 (m, 1H), 2.07-2.01 |
| | | (m, 1H), 1.80-1.71 (m, 4H). |
| A223 | mass | ¹H NMR (400 MHz, DMSO-d₆) |
| | calcd. for | δ 10.88 (s, 1H), 9.89 (s, |
| | C₄₃H₃₆N₈O₆, | 1H), 9.52-9.48 (m, 1H), 9.46 (t, |
| | 760.3; m/z | J = 5.9 Hz, 1H), 8.81 |
| | found, 761.1 | (dd, J = 8.2, 2.2 Hz, 1H), |
| | [M + H]⁺. | 8.76 (s, 1H), 8.25-8.21 (m, |
| | | 1H), 8.13 (d, J = 7.8 Hz, 1H), |
| | | 7.96-7.91 (m, 1H), 7.89 |
| | | (dd, J = 7.2, 1.3 Hz, 1H), 7.55 (s, |
| | | 1H), 7.47 (d, J = 8.4 Hz, |
| | | 1H), 7.37-7.32 (m, 1H), |
| | | 7.32-7.29 (m, 1H), 7.12 (d, J = |
| | | 7.3 Hz, 1H), 4.46 (d, J = 5.9 Hz, |
| | | 2H), 4.22 (dd, J = 12.2, |
| | | 4.9 Hz, 1H), 3.83-3.80 (m, 4H), |
| | | 3.64 (s, 3H), 3.42 (s, |
| | | 3H), 3.17-3.13 (m, 4H), |
| | | 2.74-2.66 (m, 1H), 2.56-2.52 |
| | | (m, 1H), 2.40-2.30 (m, 1H), |
| | | 2.07-2.00 (m, 1H). |
| A224 | mass | ¹H NMR (400 MHz, DMSO-d₆) |
| | calcd. for | δ 10.88 (s, 1H), 9.43 (d, J = |
| | C₄₂H₄₀N₈O₆, | 6.0 Hz, 2H), 8.69 (dd, J = 8.3, 2.0 |
| | 752.3; m/z | Hz, 1H), 8.32 (s, 2H), |
| | found, 753.5 | 8.15 (d, J = 8.2 Hz, 1H), 7.46 |
| | [M + H]⁺. | (d, J = 8.2 Hz, 1H), 7.34 (t, |
| | | J = 7.9 Hz, 1H), 7.29 (s, 1H), 7.12 |
| | | (d, J = 7.3 Hz, 1H), |
| | | 7.05 (d, J = 1.7 Hz, 1H), 6.94 |
| | | (d, J = 1.9 Hz, 1H), 4.42 (d, |
| | | J = 5.8 Hz, 2H), 4.23-4.19 |
| | | (m, 1H), 3.95-3.91 (m, 2H), |
| | | 3.88-3.81 (m, 3H), 3.63 (s, 3H), |
| | | 3.53-3.51 (m, 2H), |
| | | 3.32 (s, 3H), 2.69-2.66 (m, 1H), |
| | | 2.35-2.31 (m, 1H), |
| | | 2.08-1.98 (m, 2H), 1.78-1.72 |
| | | (m, 4H), 1.22-1.18 (m, |
| | | 3H). |
| A225 | mass | ¹H NMR (400 MHz, DMSO-d₆) |
| | calcd. for | δ 10.89 (s, 1H), 9.61- |
| | C₄₄H₄₅N₉O₆, | 9.17 (m, 2H), 8.57 (d, J = 1.9 Hz, |
| | 795.3; m/z | 1H), 8.16 (s, 1H), 7.48 |
| | found, 796.3 | (d, J = 8.3 Hz, 1H), 7.35 |
| | [M + H]⁺. | (t, J = 7.9 Hz, 1H), 7.31 (s, 1H) |
| | | 7.13 (d, J = 7.4 Hz, 1H), 6.41 |
| | | (d, J = 2.3 Hz, 1H), 6.19 (d, |
| | | J = 2.3 Hz, 1H), 4.42 (d, |
| | | J = 5.8 Hz, 2H), 4.22 (m, 1H), |
| | | 4.04-3.77 (m, 2H), |
| | | 3.53 (s, 3H), 3.52-3.44 (m, 2H), |
| | | 3.24 (s, 3H), 3.17 (s, 3H), 3.11 |
| | | (s, 6H), 2.68 (s, 3H), 2.56- |
| | | 2.54 (m, 1H), 2.38-2.32 (m, 2H), |
| | | 2.09-1.93 (m, 2H), |
| | | 1.71-1.50 (m, 4H). |
| A226 | mass | ¹H NMR (400 MHz, DMSO-d₆) |
| | calcd. for | δ 10.89 (s, 1H), 9.51- |
| | C₃₈H₃₂N₈O₅, | 9.25 (m, 2H), 8.67 (dd, J = 8.3, |
| | 680; m/z | 1.9 Hz, 1H), 8.14 (d, J = |
| | found, 680.8 | 8.2 Hz, 1H), 7.88 (s, 1H), 7.47 (d, |
| | [M + H]⁺. | J = 8.3 Hz, 1H), 7.34 (t, |
| | | J = 7.9 Hz, 1H), 7.29 (s, 1H), |
| | | 7.24 (d, J = 8.5 Hz, 2H), |
| | | 7.14-7.05 (m, 2H), 4.43 (d, J = 5.8 |
| | | Hz, 2H), 4.21 (dd, J = |
| | | 12.1, 4.8 Hz, 1H), |
| | | 3.71-3.64 (m, 2H), 3.36 (s, 3H), |
| | | 3.33 (s, 3H), 3.08-3.01 |
| | | (m, 2H), 2.74-2.66 (m, 1H), |
| | | 2.57-2.52 (m, 1H), 2.39-2.29 |
| | | (m, 1H), 2.21-2.12 (m, |
| | | 2H), 2.07-2.00 (m, 1H). |
| A227 | mass | ¹H NMR (400 MHz, DMSO-d₆) |
| | calcd. for | δ 10.88 (s, 1H), 9.24 (t, J = |
| | C₄₀H₃₆N₈O₅, | 5.9 Hz, 1H), 9.19 (s, 1H), |
| | 708.28; m/z | 8.41 (s, 1H), 7.91 (s, 1H), |
| | found, 709.28 | 7.47 (d, J = 8.3 Hz, 1H), |
| | [M + H]⁺. | 7.34 (t, J = 7.8 Hz, 1H), 7.29 (s, |
| | | 1H), 7.15-7.06 (m, 2H), 6.84 (s, |
| | | 1H), 4.40 (d, J = 5.9 Hz, |
| | | 2H), 4.22 (dd, J = 12.1, 4.9 Hz, |
| | | 1H), 3.73-3.62 (m, 2H), |
| | | 3.59 (s, 3H), 3.30 (s, 3H), |
| | | 3.03-3.00 (m, 2H), 2.73-2.63 |
| | | (m, 4H), 2.59 (s, 3H), 2.54-2.49 |
| | | (m, 1H), 2.34-2.30 (m, 1H), |
| | | 2.14-2.10 (m, 2H), 2.09-1.99 (m, 1H). |
| A228 | mass | ¹H NMR (400 MHz, DMSO-d₆) |
| | calcd. for | δ 10.92 (s, 1H), 9.50 (d, J = |
| | C₄₂H₃₅N₇O₅, | 1.6 Hz, 1H), 9.41-9.38 (m, 2H), |
| | 717.27; m/z | 8.90 (d, J = 0.8 Hz, |
| | found, 718.4 | 1H), 8.80 (dd, J = 8.4, 2.0 Hz, |
| | [M + H]⁺. | 1H), 8.77 (s, 1H), 8.23 (d, J = |
| | | 8.4 Hz, 1H), 8.19 (s, 1H), 8.10 (d, |
| | | J = 8.4 Hz, 1H), 7.94- |
| | | 7.90 (m, 1H), 7.87 (s, 1H), 7.68 (d, |
| | | J = 6.4 Hz, 1H), 7.28 |
| | | (d, J = 1.6 Hz, 1H), 7.21 (d, |
| | | J = 1.6 Hz, 1H), 4.40 (d, J = |
| | | 6.0 Hz, 2H), 4.19 (dd, J = 12.4, |
| | | 4.8 Hz, 1H), 3.78-3.72 |
| | | (m, 1H), 3.66 (s, 3H), 3.39 |
| | | (s, 3H), 2.75-2.67 (m, 1H), |
| | | 2.60-2.54 (m, 1H), 2.41-2.33 |
| | | (m, 1H), 2.13-2.06 (m, |
| | | 1H), 1.35 (d, J = 6.8 Hz, 6H). |
| A229 | mass | ¹H NMR (400 MHz, DMSO-d₆) |
| | calcd. for | δ 10.87 (s, 1H), 9.46- |
| | C₄₁H₃₈N₈O₆, | 9.40 (m, 2H), 8.74-8.68 |
| | 738.3; m/z | (m, 1H), 8.40-8.36 (m, 2H), |
| | found, 739.3 | 8.19-8.14 (m, 1H), 7 |
| | [M + H]⁺. | .49-7.44 (m, 1H), 7.36-7.31 (m, |
| | | 1H), 7.29 (s, 1H), 7.17-7.10 |
| | | (m, 1H), 7.09-7.06 (m, |
| | | 1H), 7.00-6.97 (m, 1H), |
| | | 4.45-4.40 (m, 2H), 4.23-4.18 |
| | | (m, 1H), 3.96-3.92 (m, 2H), |
| | | 3.62 (s, 3H), 3.53-3.52 (m, |
| | | 2H), 3.49-3.48 (m, 1H), 3.41 |
| | | (s, 3H), 3.31 (s, 3H), 2.73- |
| | | 2.68 (m, 1H), 2.55-2.53 |
| | | (m, 1H), 2.36-2.32 (m, 1H), |
| | | 2.06-2.01 (m, 1H), 1.78-1.71 (m, 4H). |
| A230 | mass | ¹H NMR (400 MHz, DMSO-d₆) |
| | calcd. for | δ 10.93 (s, 1H), 9.51- |
| | C₃₉H₃₀N₈O₆, | 9.48 (m, 1H), 9.46-9.45 |
| | 706.2; m/z | (m, 1H), 9.13 (s, 1H), 8.80- |
| | found, 707.2 | 8.77 (m, 2H), 8.43 (d, J = 5.8 Hz, |
| | [M + H]⁺. | 1H), 8.21 (d, J = 8.0 Hz, |
| | | 1H), 7.74 (s, 1H), 7.58 (d, |
| | | J = 5.6 Hz, 1H), 7.50 (s, 1H), |
| | | 7.39-7.38 (m, 1H), 7.32 (d, |
| | | J = 8.1 Hz, 1H), 7.27-7.25 |
| | | (m, 1H), 4.46 (d, |
| | | J = 6.0 Hz, 2H), 4.45-4.41 (m, 1H), |
| | | 3.66 (s, 3H), 3.41 (s, 3H), |
| | | 3.39 (s, 3H), 2.69-2.64 (m, |
| | | 1H), 2.58-2.56 (m, 1H), |
| | | 2.35-2.31 (m, 1H), 2.20-2.15 |
| | | (m, 1H). |
| A231 | mass | ¹H NMR (400 MHz, DMSO-d₆) |
| | calcd. for | δ 10.88 (s, 1H), 9.24 (t, J = |
| | C₃₉H₃₄N₈O₅, | 5.9 Hz, 1H), 9.20-9.18 |
| | 694.3; m/z | (m, 1H), 8.42-8.40 (m, 1H), |
| | found, 695.3 | 7.87 (s, 1H), 7.47 (d, J = 8.4 Hz, |
| | [M + H]⁺. | 1H), 7.33 (t, J = 8.1 Hz, |
| | | 1H), 7.30 (s, 1H), 7.26-7.23 (m, |
| | | 2H), 7.12 (d, J = 7.6 Hz, |
| | | 1H), 7.08 (dd, J = 8.2, 1.8 Hz, |
| | | 1H), 4.40 (d, J = 5.8 Hz, |
| | | 2H), 4.22 (dd, J = 12.3, |
| | | 4.9 Hz, 1H), 3.69-3.65 (m, 2H), |
| | | 3.36 (s, 3H), 3.33 (s, 3H), 3.04 |
| | | (t, J = 6.4 Hz, 2H), 2.75- |
| | | 2.67 (m, 1H), 2.65 (s, 3H), |
| | | 2.58-2.53 (m, 1H), 2.39- |
| | | 2.33 (m, 1H), 2.18-2.13 |
| | | (m, 2H), 2.06-2.01 (m, 1H). |
| A232 | mass | ¹H NMR (400 MHz, DMSO-d₆) |
| | calcd. For | δ 10.89 (s, 1H), 9.55- |
| | C₄₃H₃₈N₈O₄, | 9.43 (m, 2H), 9.41-9.39 |
| | 730; m/z | (m, 1H), 9.21 (s, 1H), 8.85- |
| | found, 731.5 | 8.75 (m, 2H), 8.29-8.17 |
| | [M + H]⁺. | (m, 2H), 8.11 (d, J = 8.3 Hz, |
| | | 2H), 7.95-7.89 (m, 1H), 7.69 (d, |
| | | J = 7.0 Hz, 1H), 7.33- |
| | | 7.27 (m, 1H), 7.21 (s, 1H), 4.49 |
| | | (d, J = 5.9 Hz, 2H), 4.28 |
| | | (m, 1H), 4.01 (s, 3H), |
| | | 3.76 (s, 1H), 3.66 (s, 3H), 3.39 (s, |
| | | 3H), 2.74-2.66 (m, 1H), |
| | | 2.63-2.54 (m, 1H), 2.36-2.32 |
| | | (m, 1H), 2.11-2.05 (m, 1H), |
| | | 1.35 (d, J = 6.8 Hz, 6H). |
| A233 | mass | ¹H NMR (400 MHz, DMSO-d₆) |
| | calcd. for | δ 10.98 (s, 1H), 9.55 (d, J = |
| | C₄₄H₃₅N₇O₅, | 1.7 Hz, 1H), 9.47-9.41 (m, 2H), |
| | 741.27; m/z | 8.88-8.84 (m, 1H), |
| | found, 742 | 8.82 (s, 1H), 8.28 (d, J = 8.2 |
| | [M + H]⁺. | Hz, 1H), 8.24 (s, 1H), 8.19- |
| | | 8.14 (m, 2H), 8.03-7.93 |
| | | (m, 2H), 7.77-7.73 (m, 1H), |
| | | 7.34 (d, J = 1.4 Hz, 1H), 7.27 |
| | | (d, J = 1.4 Hz, 1H), 4.47 (d, |
| | | J = 5.9 Hz, 2H), |
| | | 4.34-4.24 (m, 1H), 3.85-3.78 (m, 1H), |
| | | 3.72 (s, 3H), 3.45 (s, 3H), |
| | | 2.81-2.73 (m, 1H), 2.67-2.60 |
| | | (m, 1H), 2.46-2.37 (m, 1H), |
| | | 2.19-2.13 (m, 1H), 1.42- |
| | | 1.39 (m, 6H). |
| A234 | mass | ¹H NMR (400 MHz, DMSO-d₆) |
| | calcd. for | δ 10.88 (s, 1H), 9.55- |
| | C₄₃H₃₅FN₆O₅, | 9.46 (m, 1H), 9.44-9.34 (m, |
| | 734.2; m/z | 2H), 8.80 (dd, J = 8.2, 2.2 |
| | found, 735.3 | Hz, 1H), 8.77 (s, 1H), |
| | [M + H]⁺. | 8.28-8.17 (m, 1H), 8.13-8.07 (m, |
| | | 1H), 7.98 (s, 1H), 7.95-7.89 (m, 1H), |
| | | 7.80 (d, J = 7.1 Hz, |
| | | 1H), 7.73-7.63 (m, 2H), |
| | | 7.32-7.26 (m, 1H), 7.24-7.18 |
| | | (m, 1H), 4.42 (d, J = 5.9 Hz, |
| | | 2H), 4.15 (dd, J = 12.3, 4.8 |
| | | Hz, 1H), 3.80-3.70 (m, 1H), |
| | | 3.66 (s, 3H), 3.39 (s, 3H), |
| | | 2.73-2.66 (m, 1H), 2.59-2.53 |
| | | (m, 1H), 2.39-2.27 (m, |
| | | 1H), 2.12-2.03 (m, 1H), |
| | | 1.35 (d, J = 6.8 Hz, 6H). |
| A235 | mass | ¹H NMR (400 MHz, DMSO-d₆) |
| | calcd. for | δ 10.88 (s, 1H), 9.24- |
| | C₄₀H₃₇FN₈O₅, | 9.18 (m, 2H), 8.44 (s, 1H), |
| | 728.29; | 8.37 (d, J = 2.3 Hz, 2H), 7.98 |
| | m/z found, | (s, 1H), 7.79 (d, J = 7.1 Hz, |
| | 729.29 | 1H), 7.69-7.63 (m, 1H), 7.06 |
| | [M + H]⁺. | (s, 1H), 6.98 (s, 1H), 4.37 |
| | | (s, 2H), 4.15-4.10 (m, 1H), |
| | | 3.74-3.55 (m, 4H), 3.39-3.30 |
| | | (m, 3H), 3.31 (s, 3H), |
| | | 2.68-2.60 (m, 4H), 2.57-2.50 |
| | | (m, 1H), 2.34-2.28 (m, |
| | | 1H), 2.09-2.00 |
| | | (m, 1H), 1.30-1.25 (m, 6H). |
| A236 | mass | ¹H NMR (400 MHz, DMSO-d₆) |
| | calcd. for | δ 10.88 (s, 1H), 9.38- |
| | C₄₁H₃₈N₈O₆, | 9.16 (m, 2H), 8.77 (s, 1H), 8.65 |
| | 738; m/z | (s, 2H), 8.49 (d, J = 1.4 |
| | found, 739 | Hz, 1H), 7.48 (d, J = 8.3 |
| | [M + H]⁺. | Hz, 1H), 7.34 (dd, J = 17.6, 9.3 |
| | | Hz, 2H), 7.13 (d, J = 7.3 Hz, |
| | | 1H), 6.96 (d, J = 1.9 Hz, |
| | | 1H), 6.83 (d, J = 1.9 Hz, 1H), |
| | | 4.41 (d, J = 5.9 Hz, 2H), |
| | | 4.22 (dd, J = 12.2, 4.9 Hz, |
| | | 1H), 3.96 (d, J = 11.0 Hz, 2H), |
| | | 3.59 (s, 3H), 3.54-3.47 (m, 3H), |
| | | 3.32 (s, 3H), 2.80-2.69 |
| | | (m, 1H), 2.67 (s, 3H), 2.58-2.54 |
| | | (m, 1H), 2.38-2.32 (m, 1H), 2.07-2.01 |
| | | (m, 1H), 1.80-1.71 (m, 4H). |
| A237 | mass | ¹H NMR (400 MHz, DMSO-d₆) |
| | calcd. for | δ ¹H NMR (400 MHz, |
| | C₄₁H₃₈N₈O₅, | DMSO-d₆) δ 10.88 (s, |
| | 722.3; m/z | 1H), 9.44-9.35 (m, 2H), 8.67 (dd, |
| | found, 723.6 | J = 8.2, 2.1 Hz, 1H), |
| | [M + H]⁺. | 8.14 (d, J = 8.3 Hz, 1H), 7.88 (s, |
| | | 1H), 7.46 (d, J = 8.3 Hz, 1H), |
| | | 7.37-7.31 (m, 1H), 7.29 (s, |
| | | 1H), 7.12 (d, J = 7.4 Hz, 1H), |
| | | 7.08 (d, J = 1.9 Hz, 1H), |
| | | 7.01 (d, J = 1.9 Hz, 1H), |
| | | 4.43 (d, J = 5.9 Hz, 2H), 4.24- |
| | | 4.18 (m, 1H), 3.71-3.64 (m, 3H), |
| | | 3.59 (s, 3H), 3.31 (s, |
| | | 3H), 3.07-3.01 (m, 2H), 2.74-2.66 |
| | | (m, 1H), 2.54 (n, J = |
| | | 3.8 Hz, 1H), 2.39-2.30 |
| | | (m, 1H), 2.19-2.12 (m, 2H), |
| | | 2.06-1.99 (m, 1H), 1.30-1.26 (m, 6H). |
| A238 | mass | ¹H NMR (400 MHz, DMSO-d₆) |
| | calcd. for | δ 10.83 (s, 1H), 9.11 (t, J = |
| | C₄₂H₄₁N₉O₄, | 5.9 Hz, 1H), 9.00 (d, J = 1.8 Hz, |
| | 735; m/z | 1H), 8.61 (dd, J = 7.2, |
| | found, 736 | 0.6 Hz, 1H), 8.23 (d, |
| | [M + H]⁺. | J = 1.5 Hz, 1H), 7.95 (s, 1H), 7.81 (s, |
| | | 2H), 7.73 (s, 1H), 7.03 (d, |
| | | J = 1.9 Hz, 1H), 6.97 (d, J = 1.9 |
| | | Hz, 1H), 6.81 (dd, J = 7.2, 1.7 Hz, |
| | | 1H), 4.34 (d, J = 5.9 |
| | | Hz, 2H), 4.21 (dd, |
| | | J = 12.2, 4.8 Hz, 1H), 3.71-3.63 (m, |
| | | 3H), 3.60 (s, 3H), 3.31 (s, 3H), |
| | | 2.93-2.90 (m, 2H), 2.73- |
| | | 2.66 (m, 1H), 2.63 (s, 3H), |
| | | 2.59-2.54 (m, 1H), 2.35- |
| | | 2.28 (m, 1H), 2.07-2.01 (m, 3H), |
| | | 1.28 (d, J = 6.8 Hz, |
| | | 6H). |
| A239 | mass | ¹H NMR (400 MHz, DMSO-d₆) |
| | calcd. for | δ 10.88 (s, 1H), 9.49 (t, J = |
| | C₄₀H₃₆N₈O₅, | 5.9 Hz, 1H), 9.10 (d, J = 2.0 Hz, |
| | 708; m/z | 1H), 8.47 (dd, J = 8.2, |
| | found, 709 | 2.2 Hz, 1H), 8.26 (d, J = 8.2 Hz, |
| | [M + H]⁺. | 1H), 7.46 (d, J = 8.3 Hz, |
| | | 1H), 7.38-7.28 (m, 3H), 7.18 (s, |
| | | 1H), 7.11 (dd, J = 13.4, |
| | | 4.5 Hz, 2H), 6.85 (s, 1H), 4.45 |
| | | (d, J = 5.8 Hz, 2H), 4.21 |
| | | (dd, J = 12.2, 4.9 Hz, 1H), |
| | | 3.80 (s, 2H), 3.74 (d, J = 4.5 |
| | | Hz, 2H), 3.60 (s, 6H), 3.35 (s, 3H), |
| | | 2.73-2.66 (m, 1H), |
| | | 2.62 (s, 3H), 2.58-2.54 |
| | | (m, 1H), 2.36-2.34 (m, 1H), |
| | | 2.07-1.99 (m, 1H). |
| A240 | mass | ¹H NMR (400 MHz, DMSO-d₆) |
| | calcd. for | δ 10.92 (s, 1H), 9.51- |
| | C₄₄H₃₅N₇O₅, | 9.48 (m, 1H), 9.43-9.39 |
| | 741.3; m/z | (m, 2H), 8.82-8.78 (m, 1H), |
| | found, 742.7 | 8.76 (s, 1H), 8.30 (s, 1H), |
| | [M + H]⁺. | 8.24-8.21 (m, 2H), 8.11-8.08 |
| | | (m, 1H), 7.99 (s, 1H), 7.94-7.89 |
| | | (m, 1H), 7.70-7.67 (m, |
| | | 1H), 7.28 (s, 1H), 7.21 (d, |
| | | J = 1.1 Hz, 1H), 4.47 (d, J = 5.9 |
| | | Hz, 2H), 4.26-4.20 |
| | | (m, 1H), 3.78-3.71 (m, 1H), 3.66 (s, |
| | | 3H), 3.39 (s, 3H), 2.76-2.67 |
| | | (m, 1H), 2.62-2.55 (m, |
| | | 1H), 2.42-2.32 (m, 1H), 2.12-2.06 |
| | | (m, 1H), 1.36-1.33 |
| | | (m, 6H). |
| A241 | mass | ¹H NMR (400 MHz, DMSO-d₆) |
| | calcd. for | δ 10.88 (s, 1H), 9.27 (t, J = |
| | C₃₉H₃₄N₈O₆, | 5.9 Hz, 1H), 9.17 (d, J = 1.6 Hz, |
| | 710.26; m/z | 1H), 8.40 (d, J = 1.4 |
| | found, 711 | Hz, 1H), 8.04 (s, 1H), 7.47 |
| | [M + H]⁺. | (d, J = 8.4 Hz, 1H), 7.37-7.32 |
| | | (m, 1H), 7.30 (s, 1H), 7.14-7.11 |
| | | (m, 2H), 6.89 (d, J = 1.6 |
| | | Hz, 1H), 4.63-4.58 (m, 2H), |
| | | 4.39 (d, J = 5.9 Hz, 2H), |
| | | 4.22 (dd, J = 12.3, 4.9 Hz, |
| | | 1H), 3.81-3.77 (m, 2H), 3.59 |
| | | (s, 3H), 3.31 (s, 3H), 2.71-2.67 |
| | | (m, 1H), 2.65 (s, 3H), |
| | | 2.60 (s, 3H), 2.57-2.54 (m, 1H), |
| | | 2.36-2.31 (m, 1H), |
| | | 2.07-2.00 (m, 1H). |
| A242 | mass | ¹H NMR (400 MHz, DMSO-d₆) |
| | calcd. for | δ 10.88 (s, 1H), 9.42 (t, J = |
| | C₄₁H₃₄F₃N₇O₅, | 5.6 Hz, 1H), 8.56 (s, 1H), 8.11 |
| | 761.26; | (d, J = 8.0 Hz, 1H), 7.93 |
| | m/z found, | (d, J = 7.9 Hz, 1H), 7.47 (d, |
| | 762.26 | J = 8.2 Hz, 1H), 7.34 (t, J = |
| | [M + H]⁺. | 7.8 Hz, 1H), 7.29 (s, 1H), 7.22 (d, |
| | | J = 8.2 Hz, 1H), 7.18 (s, |
| | | 1H), 7.12 (d, J = 7.3 Hz, 1H), |
| | | 7.01 (d, J = 8.1 Hz, 1H), |
| | | 6.59 (s, 1H), 6.52 (s, 1H), |
| | | 4.43-4.41 (m, 2H), 4.25-4.18 |
| | | (m, 1H), 3.74 (s, 3H), |
| | | 3.52 (s, 3H), 2.98 (m, 3H), 2.78- |
| | | 2.64 (m, 2H), 2.55-2.51 |
| | | (m, 2H), 2.35-2.21 (m, 2H), |
| | | 2.16-1.88 (m, 2H). |
| A243 | mass | ¹H NMR (400 MHz, DMSO-d₆) |
| | calcd. for | δ 10.88 (s, 1H), 9.37- |
| | C₃₇H₃₂N₁₀O₅, | 9.34 (m, 1H), 9.33-9.29 |
| | 696.3; m/z | (m, 1H), 8.66-8.62 (m, 1H), |
| | found, 697.5 | 8.14 (d, J = 8.3 Hz, 1H), |
| | [M + H]⁺. | 8.00 (s, 1H), 7.86 (s, 1H), 7.62- |
| | | 7.59 (m, 1H), 7.40 (dd, |
| | | J = 8.7, 1.3 Hz, 1H), 7.31-7.29 |
| | | (m, 1H), 7.26-7.23 (m, 1H), |
| | | 7.14-7.10 (m, 1H), 4.65- |
| | | 4.61 (m, 2H), 4.41-4.34 |
| | | (m, 3H), 3.99 (s, 3H), 3.83- |
| | | 3.79 (m, 2H), 3.36 (s, 3H), |
| | | 3.33 (s, 3H), 2.67-2.56 (m, |
| | | 2H), 2.42-2.34 (m, 1H), |
| | | 2.17-2.10 (m, 1H). |
| A244 | mass | ¹H NMR (400 MHz, DMSO-d₆) |
| | calcd. for | δ 10.89 (s, 1H), 9.30 (t, J = |
| | C₄₄H₃₈F₄N₆O₆, | 5.6 Hz, 1H), 8.41 (d, |
| | 822.2; | J = 9.1 Hz, 2H), 7.76 (s, 1H), 7.54- |
| | m/z found, | 7.45 (m, 2H), 7.40 (d, |
| | 823.2 | J = 11.9 Hz, 1H), 7.35 (t, J = 7.8 |
| | [M + H]⁺. | Hz, 1H), 7.30 (s, 1H), 7.13 (d, |
| | | J = 7.4 Hz, 1H), 6.89 (d, J = |
| | | 3.7 Hz, 2H), 4.40 (d, J = 5.4 |
| | | Hz, 2H), 4.25-4.17 (m, |
| | | 1H), 3.95 (d, J = 10.1 Hz, 2H), |
| | | 3.59 (s, 3H), 3.54-3.48 |
| | | (m, 3H), 3.29 (s, 3H), |
| | | 2.74-2.68 (m, 1H), 2.63 (s, 3H), |
| | | 2.58-2.54 (m, 1H), 2.38-2.31 |
| | | (m, 1H), 2.08-1.99 (m, |
| | | 1H), 1.78-1.65 (m, 4H). |
| A245 | mass | ¹H NMR (400 MHz, DMSO-d₆) |
| | calcd. for | δ 11.01 (s, 1H), 9.51 (d, J = |
| | C₃₇H₃₂N₁₀O₅, | 6.1 Hz, 1H), 9.46 (s, 1H), 8.76 |
| | 696.3; m/z | (d, J = 8.2 Hz, 1H), 8.51 |
| | found, 697.3 | (d, J = 5.8 Hz, 1H), 8.23 (d, |
| | [M + H]⁺. | J = 8.2 Hz, 1H), 7.66 (d, J = |
| | | 5.8 Hz, 1H), 7.57 (s, 1H), 7.45 (s, 1H), |
| | | 7.30 (d, J = 8.4 Hz, |
| | | 2H), 7.12 (d, J = 9.6 Hz, 1H), |
| | | 4.52 (d, J = 5.9 Hz, 3H), |
| | | 3.81 (s, 3H), 3.44-3.42 |
| | | (m, 4H), 3.42-3.40 (m, 6H), |
| | | 2.69-2.64 (m, 2H), 2.45-2.41 |
| | | (m, 1H), 2.28-2.23 (m, |
| | | 1H) |
| A246 | mass | ¹H NMR (400 MHz, DMSO-d₆) |
| | calcd. for | δ 10.82 (s, 1H), 9.37 (s, |
| | C₃₆H₃₀N₁₀O₅, | 2H), 8.62 (dd, J = 15.8, 7.7 Hz, 2H), |
| | 682.24; | 8.14 (d, J = 8.3 Hz, |
| | m/z found, | 1H), 7.97 (d, J = 25.7 Hz, 2H), |
| | 683.24 | 7.81 (s, 1H), 7.32-7.22 |
| | [M + H]⁺. | (m, 2H), 7.12 (d, J = 8.3 Hz, 1H), |
| | | 6.80 (d, J = 7.2 Hz, 1H), |
| | | 4.63 (s, 2H), 4.38-4.32 |
| | | (m, 2H), 4.20-4.11 (m, 1H), |
| | | 3.81-3.75 (m, 2H), 3.36-3.30 |
| | | (m, 6H), 2.70-2.63 (m, |
| | | 1H), 2.58-2.52 (m, 1H), |
| | | 2.31-2.22 (m, 1H), 2.05-1.98 |
| | | (m, 1H). |
| A247 | mass | ¹H NMR (400 MHz, DMSO-d₆) |
| | calcd. for | δ 10.91 (s, 1H), 9.45 (t, J = |
| | C₃₇H₃₀N₈O₆, | 5.9 Hz, 1H), 9.36 (s, 1H), |
| | 682.2; m/z | 8.69-8.60 (m, 1H), 8.14 (d, J = |
| | found, 683.3 | 8.2 Hz, 1H), 8.00 (s, 1H), |
| | [M + H]⁺. | 7.59-7.51 (m, 1H), 7.31- |
| | | 7.23 (m, 5H), 7.17-7.07 |
| | | (m, 1H), 4.67-4.60 (m, 2H), |
| | | 4.43 (d, J = 5.8 Hz, 2H), 4.27 |
| | | (dd, J = 12.6, 4.9 Hz, 1H), |
| | | 3.84-3.78 (m, 2H), 3.36 |
| | | (s, 3H), 3.32 (s, 3H), 2.85-2.75 |
| | | (m, 1H), 2.59-2.53 (m, 1H), |
| | | 2.42-2.30 (m, 1H), 2.09- |
| | | 2.01 (m, 1H). |
| A248 | mass | ¹H NMR (400 MHz, DMSO-d₆) |
| | calcd. for | δ 10.89 (s, 1H), 9.49 (d, J = |
| | C₄₄H₃₈N₆O₅, | 1.6 Hz, 1H), 9.40 (s, 1H), |
| | 730.29; m/z | 9.32 (t, J = 5.9 Hz, 1H), 8.84- |
| | found, 731.29 | 8.70 (m, 2H), 8.22 (d, |
| | [M + H]⁺. | J = 8.4 Hz, 1H), 8.10 (d, J = 8.4 |
| | | Hz, 1H), 7.96-7.87 (m, 1H), |
| | | 7.68 (d, J = 7.1 Hz, 1H), |
| | | 7.48 (d, J = 8.2 Hz, 2H), |
| | | 7.33-7.25 (m, 2H), 7.21 (d, J = |
| | | 1.4 Hz, 1H), 4.37 (d, J = 5.9 |
| | | Hz, 2H), 4.13 (dd, J = 13.2, |
| | | 5.0 Hz, 1H), 3.82-3.71 (m, 1H), |
| | | 3.66 (s, 3H), 3.37 (s, |
| | | 3H), 2.86-2.65 (m, 1H), |
| | | 2.54-2.50 (m, 1H), 2.46- |
| | | 2.28 (m, 4H), 1.96-1.90 |
| | | (m, 1H), 1.35 (d, J = 6.8 Hz, |
| | | 6H). |
| A249 | mass | ¹H NMR (400 MHz, DMSO-d₆) |
| | calcd. for | δ 10.86 (s, 1H), 9.49 (d, J = |
| | C₄₂H₃₈N₈O₄, | 1.6 Hz, 1H), 9.40 (s, 1H), 9.33 |
| | 718.3; m/z | (t, J = 6.0 Hz, 1H), 8.82- |
| | found, 719.4 | 8.75 (m, 2H), 8.21 (d, J = 8.4 Hz, |
| | [M + H]⁺. | 1H), 8.10 (d, J = 8.4 |
| | | Hz, 1H), 7.94-7.89 (m, 1H), |
| | | 7.69 (dd, J = 8.4, 4.4 Hz, |
| | | 2H), 7.28 (d, J = 1.6 Hz, |
| | | 1H), 7.21 (d, J = 1.6 Hz, 1H), |
| | | 6.81 (d, J = 1.6 Hz, 1H), |
| | | 4.69 (dd, J = 13.2, 4.8 Hz, 1H), |
| | | 4.36 (d, J = 6.0 Hz, 2H), |
| | | 3.79-3.71 (m, 1H), 3.66 (s, 3H), |
| | | 3.56-3.49 (m, 1H), 3.39 |
| | | (s, 3H), 3.06-2.98 (m, 2H), |
| | | 2.75-2.67 (m, 1H), 2.65-2.60 |
| | | (m, 2H), 2.27-2.22 (m, |
| | | 1H), 1.98-1.91 (m, 1H), |
| | | 1.35 (d, J = 6.8 Hz, 6H). |
| A251 | mass | ¹H NMR (400 MHz, DMSO-d₆) |
| | calcd. for | δ 10.96 (s, 1H), 9.32 (t, J = |
| | C₃₇H₃₁N₉O₆, | 5.8 Hz, 1H), 9.18 (d, J = 1.8 Hz, |
| | 697.2; m/z | 1H), 8.40 (d, J = 1.3 |
| | found, 698.2 | Hz, 1H), 8.34 (d, J = 5.3 Hz, 1H), |
| | [M + H]⁺. | 8.00 (s, 1H), 7.63 (d, J = |
| | | 5.3 Hz, 1H), 7.38 (s, 1H), 7.30 (d, |
| | | J = 1.8 Hz, 1H), 7.25 |
| | | (d, J = 8.3 Hz, 1H), 7.12 (dd, |
| | | J = 8.2, 1.9 Hz, 1H), 4.65- |
| | | 4.60 (m, 2H), 4.51 (dd, |
| | | J = 10.8, 5.2 Hz, 1H), 4.43 (d, J = |
| | | 5.8 Hz, 2H), 3.83-3.79 (m, 2H), |
| | | 3.37 (s, 6H), 2.80-2.73 |
| | | (m, 1H), 2.65 (s, 3H), 2.61-2.57 |
| | | (m, 1H), 2.44-2.39 (m, |
| | | 1H), 2.22-2.14 (m, 1H). |
| A252 | mass | ¹H NMR (400 MHz, DMSO-d₆) |
| | calcd. for | δ 10.88 (s, 1H), 9.30- |
| | C₄₃H₄₀N₈O₇, | 9.24 (m, 1H), 9.20-9.15 |
| | 780.3; m/z | (m, 1H), 8.43-8.39 (m, 1H), |
| | found, 781.5 | 8.04 (s, 1H), 7.47 (d, |
| | [M + H]⁺. | J = 8.3 Hz, 1H), 7.37-7.32 (m, 1H), |
| | | 7.30 (s, 1H), 7.15-7.11 |
| | | (m, 2H), 7.07-7.05 (m, 1H), |
| | | 4.64-4.59 (m, 2H), 4.40 (d, |
| | | J = 5.8 Hz, 2H), 4.24-4.19 |
| | | (m, 1H), 3.97-3.92 (m, |
| | | 2H), 3.85-3.80 (m, 2H), 3.62 (s, |
| | | 3H), 3.55-3.48 (m, 3H), |
| | | 3.34-3.33 (m, 3H), 2.71-2.64 |
| | | (m, 4H), 2.57-2.53 (m, 1H), |
| | | 2.38-2.30 (m, 1H), 2.07- |
| | | 2.01 (m, 1H), 1.82-1.72 (m, 4H). |
| A253 | mass | ¹H NMR (400 MHz, DMSO-d₆) |
| | calcd. for | δ 10.88 (s, 1H), 9.87 (s, |
| | C₄₅H₃₈N₈O₆, | 1H), 9.52-9.49 (m, 1H), |
| | 786.3; m/z | 9.48-9.42 (m, 1H), 8.86-8.80 |
| | found, 787.3 | (m, 1H), 8.76 (s, 1H), |
| | [M + H]⁺. | 8.26-8.21 (m, 1H), 8.15-8.10 (m, |
| | | 1H), 7.96-7.91 (m, 1H), |
| | | 7.90-7.83 (m, 1H), 7.57 (s, |
| | | 1H), 7.49-7.45 (m, 1H), |
| | | 7.37-7.32 (m, 1H), 7.31 (s, |
| | | 1H), 7.14-7.10 (m, 1H), |
| | | 4.51-4.43 (m, 2H), 4.42-4.37 |
| | | (m, 2H), 4.24-4.19 (m, 1H), 3.71 |
| | | (s, 3H), 3.42 (s, 3H), |
| | | 3.26-3.22 (m, 2H), 3.09-3.03 |
| | | (m, 2H), 2.74-2.67 (m, |
| | | 1H), 2.60-2.53 (m, 1H), |
| | | 2.38-2.30 (m, 1H), 2.16-2.08 |
| | | (m, 2H), 2.05-1.99 (m, 1H), |
| | | 1.94-1.83 (m, 2H). |
| A254 | mass | ¹H NMR (400 MHz, DMSO-d₆) |
| | calcd. for | δ 10.90 (s, 1H), 9.49 (d, J = |
| | C₄₃H₃₅FN₆O₅, | 2.2 Hz, 1H), 9.43-9.31 |
| | 734.3; m/z | (m, 1H), 8.84-8.72 (m, 2H), |
| | found, 735.3 | 8.28-8.09 (m, 2H), 8.08 (d, |
| | [M + H]⁺. | J = 8.0 Hz, 1H), 7.92 (dd, J = |
| | | 8.2, 7.1 Hz, 2H), 7.69 (dd, |
| | | J = 7.1, 1.1 Hz, 1H), 7.59 (d, |
| | | J = 1.4 Hz, 1H), 7.37-7.24 |
| | | (m, 2H), 7.21 (d, J = 1.6 Hz, |
| | | 1H), 4.39 (d, J = 5.9 Hz, 2H), |
| | | 4.19 (dd, J = 12.4, 4.8 Hz, |
| | | 1H), 3.83-3.69 (m, 1H), 3.66 |
| | | (s, 3H), 3.39 (s, 3H), 2.75- |
| | | 2.66 (m, 1H), 2.63-2.52 |
| | | (m, 1H), 2.40-2.24 (m, 1H), |
| | | 2.19-2.01 (m, 1H), |
| | | 1.35 (d, J = 7.6 Hz, 6H). |
| A255 | mass | ¹H NMR (400 MHz, DMSO-d₆) |
| | calcd. for | δ 11.09 (s, 1H), 9.50 (d, J = |
| | C₃₉H₃₀N₈O₄, | 1.8 Hz, 1H), 9.45-9.32 |
| | 674.2; m/z | (m, 2H), 8.87-8.72 (m, 2H), |
| | found, 675.2 | 8.23 (d, J = 8.2 Hz, 1H), |
| | [M + H]⁺. | 8.15 (s, 1H), 8.10 (d, J = 8.3 Hz, |
| | | 1H), 7.92 (t, J = 7.6 Hz, 1H), 7.82 |
| | | (s, 1H), 7.78 (d, J = 8.4 |
| | | Hz, 1H), 7.68 (d, J = 7.1 Hz, 1H), |
| | | 7.45 (s, 1H), 7.35 (d, J = |
| | | 8.0 Hz, 1H), 7.30 (d, |
| | | J = 8.1 Hz, 1H), 7.19 (d, J = 8.4 |
| | | Hz, 1H), 5.98-5.77 (m, 1H), 4.41 |
| | | (d, J = 5.9 Hz, 2H), |
| | | 3.41 (d, J = 11.1 Hz, 6H), |
| | | 2.83-2.68 (m, 3H), 2.29-2.20 |
| | | (m, 1H). |
| A256 | mass | ¹H NMR (400 MHz, DMSO-d₆) |
| | calcd. for | δ 10.88 (s, 1H), 9.29- |
| | C₄₂H₄₀N₈O₆, | 9.23 (m, 2H), 8.47-8.45 |
| | 752.3; m/z | (m, 1H), 8.38 (s, 2H), 7.49- |
| | found, 753.5 | 7.46 (m, 1H), 7.37-7.32 |
| | [M + H]⁺. | (m, 1H), 7.30 (s, 1H), 7.14- |
| | | 7.11 (m, 1H), 7.09 (s, 1H), 6.99 |
| | | (s, 1H), 4.40 (d, J = 5.8 |
| | | Hz, 2H), 4.25-4.19 (m, 1H), |
| | | 3.96-3.91 (m, 3H), 3.62 (s, |
| | | 3H), 3.54-3.48 (m, 4H), 3.40 |
| | | (s, 3H), 2.77-2.69 (m, |
| | | 1H), 2.67-2.64 (m, 3H), |
| | | 2.56-2.54 (m, 1H), 2.35-2.32 |
| | | (m, 1H), 2.06-2.01 (m, 1H), |
| | | 1.77-1.73 (m, 5H). |
| A257 | mass | ¹H NMR (400 MHz, DMSO-d₆) |
| | calcd. for | δ 10.89 (s, 1H), 9.30 (t, J = |
| | C₄₄H₃₉F₃N₆O₆, | 5.9 Hz, 1H), 8.47-8.43 (m, 1H), |
| | 804.3; | 8.27 (s, 1H), 7.78 (d, J = |
| | m/z found, | 1.6 Hz, 1H), 7.48 (d, |
| | 805.3 | J = 8.4 Hz, 2H), 7.40-7.33 (m, |
| | [M + H]⁺. | 2H), 7.31-7.28 (m, 1H), 7.13 (d, |
| | | J = 7.3 Hz, 1H), 6.98- |
| | | 6.71 (m, 3H), 4.41 (d, J = 5.9 |
| | | Hz, 2H), 4.22 (dd, J = 12.2, |
| | | 4.9 Hz, 1H), 3.96-3.93 (m, 2H), |
| | | 3.59 (s, 3H), 3.55-3.48 |
| | | (m, 3H), 3.29 (s, 3H), |
| | | 2.75-2.67 (m, 1H), 2.65 (s, 3H), |
| | | 2.57-2.52 (m, 1H), 2.39-2.31 |
| | | (m, 1H), 2.08-2.02 (m, |
| | | 1H), 1.80-1.67 (m, 4H). |
| A258 | mass | ¹H NMR (400 MHz, DMSO-d₆) |
| | calcd. for | δ 10.88 (s, 1H), 9.90 (s, |
| | C₄₅H₄₀N₈O₆, | 1H), 9.47 (dd, J = 12.5, 6.2 Hz, |
| | 788.9; m/z | 2H), 8.82 (d, J = 8.0 Hz, |
| | found, 789.6 | 1H), 8.76 (s, 1H), 8.23 (d, J = 8.3 Hz, |
| | [M + H]⁺. | 1H), 8.12 (d, J = 7.6 |
| | | Hz, 1H), 7.95-7.87 (m, 2H), |
| | | 7.51-7.46 (m, 2H), 7.34 |
| | | (dd, J = 15.3, 7.4 Hz, 2H), 7.12 |
| | | (d, J = 7.4 Hz, 1H), 4.46 |
| | | (d, J = 5.8 Hz, 2H), 4.28 |
| | | (s, 1H), 4.22 (dd, J = 12.3, 4.8 |
| | | Hz, 1H), 3.63 (s, 3H), 3.41 (s, 3H), |
| | | 3.22 (d, J = 10.3 Hz, |
| | | 2H), 3.14 (s, 2H), 2.67 (s, 1H), |
| | | 2.34 (d, J = 10.6 Hz, 2H), |
| | | 2.05 (s, 1H), 1.71 (dd, |
| | | J = 25.4, 15.2 Hz, 4H), 1.20 (s, |
| | | 3H). |
| A259 | mass | ¹H NMR (400 MHz, DMSO) |
| | calcd. for | δ 10.88 (s, 1H), 9.46 (t, J = |
| | C₄₁H₃₄N₆O₆, | 5.9 Hz, 1H), 9.35 (d, J = 1.6 Hz, |
| | 706.8; m/z | 1H), 8.66 (dd, J = 8.2, |
| | found, 707.5 | 2.1 Hz, 1H), 8.30 (s, 1H), 8.15 |
| | [M + H]⁺. | (d, J = 8.3 Hz, 1H), 7.76 (s, |
| | | 1H), 7.47 (d, J = 8.3 Hz, 1H), |
| | | 7.36-7.32 (m, 1H), 7.30 (s, |
| | | 1H), 7.26-7.21 (m, 2H), |
| | | 7.15-7.10 (m, 2H), 5.91 (s, |
| | | 1H), 4.43 (d, J = 5.9 Hz, |
| | | 2H), 4.21 (dd, J = 12.2, 4.9 Hz, |
| | | 1H), 3.37 (d, J = 4.2 Hz, 6H), |
| | | 2.76-2.67 (m, 1H), 2.56- |
| | | 2.52 (m, 1H), 2.39-2.30 |
| | | (m, 1H), 2.07-2.00 (m, 1H), |
| | | 1.55 (s, 6H). |
| A260 | mass | ¹H NMR (400 MHz, DMSO) |
| | calcd. for | δ 10.89 (s, 1H), 9.92-9.87 |
| | C₄₄H₃₈N₈O₆, | (m, 1H), 9.49 (s, 1H), 9.46 (t, J = 5.9 |
| | 774.8; m/z | Hz, 1H), 8.81 (dd, J = |
| | found, 775.5 | 8.2, 1.6 Hz, 1H), 8.74 (s, |
| | [M + H]⁺. | 1H), 8.23 (d, J = 8.2 Hz, 1H), |
| | | 8.14-8.10 (m, 1H), 7.95-7.89 |
| | | (m, 2H), 7.60 (s, 1H), |
| | | 7.47 (d, J = 8.3 Hz, 1H), 7.35 (t, |
| | | J = 7.9 Hz, 1H), 7.31 (s, |
| | | 1H), 7.13 (d, J = 7.4 Hz, |
| | | 1H), 4.47 (d, J = 5.8 Hz, 2H), |
| | | 4.22 (dd, J = 12.2, 4.8 Hz, 1H), |
| | | 4.00-3.93 (m, 2H), 3.84 |
| | | -3.79 (m, 4H), 3.65 (s, 3H), |
| | | 3.17-3.12 (m, 4H), 2.75- |
| | | 2.67 (m, 1H), 2.58-2.54 |
| | | (m, 1H), 2.39-2.30 (m, 1H), |
| | | 2.08-2.01 (m, 1H), 1.26 |
| | | (t, J = 7.1 Hz, 3H). |
| A261 | mass | ¹H NMR (400 MHz, DMSO-d₆) |
| | calcd. for | δ 10.88 (s, 1H), 9.48 (d, J = |
| | C₄₃H₃₄N₆O₅, | 7.1 Hz, 2H), 9.36 (s, 1H), 8.78 |
| | 714.8; m/z | (d, J = 8.0 Hz, 1H), 8.73 |
| | found, 715.6 | (s, 1H), 8.22 (d, J = 8.1 Hz, 1H), |
| | [M + H]⁺. | 8.08 (d, J = 7.8 Hz, 1H), |
| | | 7.89 (t, J = 7.4 Hz, 1H), 7.64 (d, |
| | | J = 6.7 Hz, 1H), 7.47 (d, |
| | | J = 8.2 Hz, 1H), 7.34 (t, J = 7.6 Hz, |
| | | 1H), 7.28 (d, J = 9.7 |
| | | Hz, 2H), 7.12 (d, J = 7.2 Hz, |
| | | 1H), 6.96 (s, 1H), 4.46 (d, J = |
| | | 4.6 Hz, 2H), 4.26-4.19 (m, 1H), |
| | | 3.78 (s, 3H), 3.37 (s, |
| | | 3H), 2.70 (d, J = 12.8 Hz, 1H), 2.55 |
| | | (s, 1H), 2.47-2.43 |
| | | (m, 1H), 2.34 (d, J = 11.6 Hz, 1H), |
| | | 2.05 (s, 1H), 0.99 (d, J = |
| | | 7.7 Hz, 2H), 0.85 (s, 2H). |
| A262 | mass | ¹H NMR (400 MHz, DMSO-d₆) |
| | calcd. for | δ 10.85 (s, 1H), 9.15 (d, J = |
| | C₃₉H₃₂N₈O₆, | 10.3 Hz, 2H), 8.38 (s, 1H), 7.98 |
| | 708.24; m/z | (d, J = 16.3 Hz, 2H), |
| | found, 709 | 7.61-7.56 (m, 2H), 7.37 (d, |
| | [M + H]⁺. | J = 10.0 Hz, 1H), 7.30 (d, J = |
| | | 1.9 Hz, 1H), 7.26-7.22 (m, |
| | | 1H), 7.12 (dd, J = 8.3, 1.9 |
| | | Hz, 1H), 4.66-4.59 (m, 2H), |
| | | 4.35-4.27 (m, 2H), 3.84- |
| | | 3.78 (m, 2H), 3.37 (s, 3H), |
| | | 3.33 (s, 3H), 3.05-3.01 (m, |
| | | 1H), 2.94-2.86 (m, 2H), |
| | | 2.79-2.72 (m, 2H), 2.63 (s, |
| | | 3H). |
| A263 | mass | ¹H NMR (400 MHz, DMSO-d₆) |
| | calcd. for | δ 10.88 (s, 1H), 9.39- |
| | C₄₁H₃₄N₆O₆, | 9.24 (m, 2H), 8.66 (dd, J = 8.2, |
| | 706; m/z | 2.2 Hz, 1H), 8.30 (s, 1H), |
| | found, 707 | 8.14 (d, J = 8.2 Hz, 1H), |
| | [M + H]⁺. | 7.96 (s, 1H), 7.82-7.69 (m, 2H), |
| | | 7.57 (d, J = 8.5 Hz, 1H), 7.36 (dd, |
| | | J = 8.6, 1.6 Hz, 1H), |
| | | 7.24 (d, J = 8.2 Hz, 2H), 7.12 |
| | | (dd, J = 7.9, 1.6 Hz, 1H), |
| | | 5.90 (s, 1H), 4.36 (d, J = 5.9 Hz, |
| | | 2H), 4.15 (dd, J = 12.2, |
| | | 4.8 Hz, 1H), 3.37 (d, J = 4.3 Hz, |
| | | 6H), 2.77-2.67 (m, 1H), |
| | | 2.59-2.53 (m, 1H), 2.37-2.28 |
| | | (m, 1H), 2.12-2.04 (m, |
| | | 1H), 1.55 (s, 6H). |
| A264 | mass | ¹H NMR (400 MHz, DMSO-d₆) |
| | calcd. for | δ 10.88 (s, 1H), 9.45 (t, J = |
| | C₃₉H₃₄N₈O₆, | 5.9 Hz, 1H), 9.37 (s, 1H), 8.67 (dd, |
| | 710.3; m/z | J = 8.2, 2.0 Hz, 1H), |
| | found, 711.4 | 8.14 (d, J = 8.2 Hz, 1H), 8.10 |
| | [M + H]⁺. | (s, 1H), 7.46 (d, J = 8.3 Hz, |
| | | 1H), 7.33 (dd, J = 18.0, 9.5 Hz, |
| | | 3H), 7.24 (d, J = 8.3 Hz, |
| | | 1H), 7.14-7.10 (m, 2H), 4.42 |
| | | (d, J = 5.8 Hz, 2H), 4.21 |
| | | (dd, J = 12.2, 4.9 Hz, 1H), |
| | | 3.64 (s, 2H), 3.36 (s, 3H), 3.34 |
| | | (s, 3H), 2.73-2.66 (m, 1H), |
| | | 2.57-2.52 (m, 1H), 2.39- |
| | | 2.32 (m, 1H), 2.07-1.99 |
| | | (m, 1H), 1.48 (s, 6H). |
| A265 | mass | ¹H NMR (400 MHz, DMSO-d₆) |
| | calcd. for | δ 10.88 (s, 1H), 9.46 (t, J = |
| | C₃₇H₂₉FN₈O₆, | 6.0 Hz, 1H), 9.38 (d, J = 1.5 Hz, |
| | 700.2; m/z | 1H), 8.67 (dd, J = 8.2, |
| | found, 701.3 | 2.1 Hz, 1H), 8.16 (t, J = 4.1 Hz, |
| | [M + H]⁺. | 2H), 7.46 (d, J = 8.3 Hz, |
| | | 1H), 7.34 (t, J = 7.9 Hz, 1H), 7.29 (s, |
| | | 1H), 7.19 (d, J = 1.6 |
| | | Hz, 1H), 7.13-7.07 |
| | | (m, 2H), 4.63-4.59 (m, 2H), 4.42 |
| | | (d, J = 5.9 Hz, 2H), 4.21 (dd, |
| | | J = 12.3, 4.7 Hz, 1H), 3.84- |
| | | 3.79 (m, 2H), 3.49 (s, 3H), 3.37 |
| | | (s, 3H), 2.69-2.65 (m, |
| | | 1H), 2.54 (d, J = 2.6 Hz, 1H), |
| | | 2.33 (dd, J = 3.5, 1.8 Hz, |
| | | 1H), 2.06-2.00 (m, 1H). |
| A266 | mass | ¹H NMR (400 MHz, DMSO-d₆) |
| | calcd. for | δ 10.88 (s, 1H), 9.28 (t, J = |
| | C₃₇H₂₉FN₈O₆, | 5.9 Hz, 1H), 9.18 (s, 1H), |
| | 697.2; m/z | 8.46 (s, 1H), 8.41 (s, 1H), |
| | found, 698.2 | 8.07 (d, J = 1.9 Hz, 1H), 7.99 |
| | [M + H]⁺. | (s, 1H), 7.72 (d, J = 1.9 Hz, |
| | | 1H), 7.47 (d, J = 8.2 Hz, 1H), |
| | | 7.34 (t, J = 7.9 Hz, 1H), |
| | | 7.30 (s, 1H), 7.12 (d, J = 7.4 Hz, |
| | | 1H), 4.69-4.56 (m, 2H), |
| | | 4.40 (d, J = 5.6 Hz, 2H), |
| | | 4.22 (dd, J = 12.0, 4.8 Hz, 1H), |
| | | 3.88-3.76 (m, 2H), 3.37 (s, 3H), |
| | | 3.35 (s, 3H), 2.72-2.68 |
| | | (m, 1H), 2.65 (s, 3H), 2.57-2.55 |
| | | (m, 1H), 2.36-2.29 (m, |
| | | 1H), 2.12-1.98 (m, 1H). |
| A267 | mass | ¹H NMR (400 MHz, DMSO-d₆) |
| | calcd. for | δ 10.88 (s, 1H), 9.53- |
| | C₄₁H₃₂N₆O₅, | 9.43 (m, 2H), 9.40 (s, 1H), |
| | 688.2; m/z | 8.84-8.74 (m, 2H), 8.23 (d, J = |
| | found, 689.3 | 8.2 Hz, 1H), 8.10 (d, J = 8.3 Hz, |
| | [M + H]⁺. | 1H), 7.95-7.88 (m, |
| | | 1H), 7.69 (d, J = 7.1 Hz, |
| | | 1H), 7.52-7.44 (m, 2H), 7.38- |
| | | 7.28 (m, 4H), 7.12 (d, J = 7.3 Hz, |
| | | 1H), 4.45 (d, J = 5.8 Hz, |
| | | 2H), 4.21 (dd, J = 12.2, 4.8 Hz, |
| | | 1H), 3.96 (q, J = 7.0 Hz, |
| | | 2H), 3.43 (s, 3H), 2.74-2.66 |
| | | (m, 1H), 2.56-2.53 (m, |
| | | 1H), 2.37-2.31 (m, 1H), 2.08-2.00 |
| | | (m, 1H), 1.24 (t, J = |
| | | 7.1 Hz, 3H). |
| A268 | mass | ¹H NMR (400 MHz, DMSO-d₆) |
| | calcd. for | δ 10.88 (s, 1H), 9.45 (t, J = |
| | C₃₆H₂₉N₉O₆, | 5.9 Hz, 1H), 9.37 (d, J = 1.6 Hz, |
| | 683.2; m/z | 1H), 8.66 (dd, J = 8.2, |
| | found, 684.2 | 2.1 Hz, 1H), 8.16 (d, J = 8.0 Hz, |
| | [M + H]⁺. | 1H), 8.08 (d, J = 2.1 Hz, |
| | | 1H), 8.00 (s, 1H), 7.72 (d, J = 2.1 Hz, |
| | | 1H), 7.46 (d, J = 8.4 |
| | | Hz, 1H), 7.36-7.32 (m, 1H), 7.29 (s, |
| | | 1H), 7.12 (d, J = 7.6 |
| | | Hz, 1H), 4.67-4.61 (m, |
| | | 2H), 4.42 (d, J = 5.9 Hz, 2H), |
| | | 4.21 (dd, J = 12.2, 4.8 Hz, 1H), |
| | | 3.86-3.79 (m, 2H), 3.38 |
| | | (s, 3H), 3.35 (s, 3H), 2.67 (dd, |
| | | J = 4.8, 3.0 Hz, 1H), 2.55 |
| | | (dd, J = 5.2, 2.6 Hz, 1H), |
| | | 2.33 (dt, J = 3.9, 1.9 Hz, 1H), |
| | | 2.07-2.00 (m, 1H) |
| A269 | mass | ¹H NMR (400 MHz, DMSO-d₆) |
| | calcd. for | δ 10.89 (s, 1H), 9.38 (s, |
| | C₄₄H₄₀N₈O₄, | 1H), 9.29 (s, 1H), 9.21-9.12 |
| | 744.3; m/z | (m, 1H), 8.71 (s, 1H), 8.55 |
| | found, | (s, 1H), 8.12-8.06 (m, |
| | 745.4 | 1H), 7.93-7.86 (m, 2H), 7.69- |
| | [M + H]⁺. | 7.59 (m, 2H), 7.45-7.40 |
| | | (m, 1H), 7.27 (s, 1H), 7.20 (s, |
| | | 1H), 4.42-4.35 (m, 3H), |
| | | 4.00 (s, 3H), 3.76-3.75 (m, |
| | | 1H), 3.66 (s, 3H), 3.38 (s, 3H), |
| | | 2.72-2.62 (m, 5H), 2.42- |
| | | 2.35 (m, 1H), 2.19-2.13 |
| | | (m, 1H), 1.34 (d, J = 6.7 Hz, |
| | | 6H). |
| A270 | mass | ¹H NMR (400 MHz, DMSO-d₆) |
| | calcd. for | δ 10.76 (s, 1H), 9.23 (d, J = |
| | C₄₀H₄₁N₉O₄, | 2.0 Hz, 1H), 9.16 (d, J = 6.0 Hz, |
| | 711.3; m/z | 1H), 8.52 (dd, J = 8.3, |
| | found, | 2.1 Hz, 1H), 8.06 (d, |
| | 712.4 | J = 8.3 Hz, 1H), 7.58 (s, 1H), 7.21 (s, |
| | [M + H]⁺. | 1H), 7.05 (t, J = 7.8 Hz, |
| | | 1H), 6.97 (d, J = 1.9 Hz, 1H), |
| | | 6.91 (d, J = 1.9 Hz, 1H), |
| | | 6.71-6.66 (m, 2H), 6.62 (d, J = |
| | | 7.5 Hz, 1H), 5.99 (d, J = 7.9 Hz, |
| | | 1H), 4.40-4.29 (m, 3H), |
| | | 3.74-3.69 (m, 2H), 3.64 |
| | | (dd, J = 13.4, 6.7 Hz, 1H), 3.58 |
| | | (s, 3H), 3.54-3.51 (m, 2H), |
| | | 3.30 (s, 3H), 3.11 (s, 3H), |
| | | 2.74 (td, J = 12.2, 6.2 Hz, 1H), |
| | | 2.59-2.53 (m, 1H), 2.07 |
| | | (dd, J = 8.7, 4.5 Hz, 1H), 1.86 |
| | | (dd, J = 12.4, 4.5 Hz, 1H), |
| | | 1.27 (d, J = 6.8 Hz, 6H). |
| A271 | mass | ¹H NMR (400 MHz, DMSO-d₆) |
| | calcd. for | δ 10.77 (s, 1H), 9.49 (d, J = |
| | C₄₄H₄₂N₈O₅, | 1.9 Hz, 1H), 9.43 (s, 1H), |
| | 762.3; m/z | 9.31 (t, J = 6.0 Hz, 1H), 8.82- |
| | found, | 8.75 (m, 2H), 8.21 (d, J = 8.3 |
| | 763.4 | Hz, 1H), 8.09 (d, J = 8.3 |
| | [M + H]⁺. | Hz, 1H), 7.93-7.88 (m, 1H), |
| | | 7.69 (d, J = 7.1 Hz, 1H), |
| | | 7.19 (d, J = 1.2 Hz, 1H), 7.06 |
| | | (dd, J = 13.5, 5.5 Hz, 2H), |
| | | 6.72 (s, 1H), 6.68 (d, J = 8.2 Hz, |
| | | 1H), 6.63 (d, J = 7.6 Hz, |
| | | 1H), 6.00 (s, 1H), 4.37 (dd, |
| | | J = 16.8, 5.3 Hz, 3H), 3.69 (s, |
| | | 3H), 3.37 (s, 3H), 3.08 (t, |
| | | J = 10.6 Hz, 2H), 2.92 (d, J = |
| | | 21.0 Hz, 2H), 2.73 |
| | | (dt, J = 11.1, 5.4 Hz, 1H), 2.59-2.53 |
| | | (m, 1H), 2.12-2.04 (m, 1H), |
| | | 1.91-1.83 (m, 1H), 1.67 |
| | | (d, J = 42.5 Hz, 4H), |
| | | 1.19 (s, 3H) |

### Example B1. N-(3-(1-(2,6-Dioxopiperidin-3-yl)-3-methyl-1H-indazol-6-yl)prop-2-yn-1-yl)-5-(8-(7-ethyl-1,3-dimethyl-2-oxo-1,2-dihydroquinolin-5-yl)isoquinolin-3-yl)picolinamide

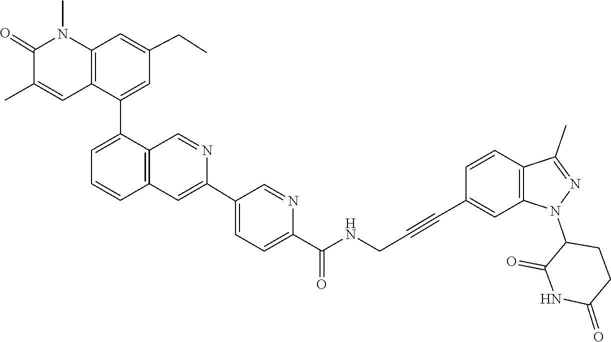 [SMILES: CCc1cc(-c2cccc3cc(-c4ccc(C(=O)NCC#Cc5ccc6c(C)nn(C7CCC(=O)NC7=O)c6c5)nc4)ncc23)c2cc(C)c(=O)n(C)c2c1]

N-(3-(1-(2,6-Dioxopiperidin-3-yl)-3-methyl-1H-indazol-6-yl)prop-2-yn-1-yl)-5-(8-(7-ethyl-1,3-dimethyl-2-oxo-1,2-dihydroquinolin-5-yl)isoquinolin-3-yl)picolinamide was prepared from 6-bromo-3-methyl-1H-indazole according to the procedures described in Example A1, Steps A D. In the final step, Lithium 5-(8-(7-ethyl-1,3-dimethyl-2-oxo-1,2-dihydroquinolin-5-yl)isoquinolin-3-yl)picolinate (Intermediate 4) was used as the coupling partner. The title compound (14.7 mg, 12% yield over 4 steps) was obtained as a white solid.

LC-MS (ESI): mass calcd. for C₄₄H₃₇N₇O₄, 727.3; m/z found, 728.1 [M+H]⁺.

¹H NMR (400 MHz, DMSO-d₆) δ 11.05 (s, 1H), 9.46 (d, J=1.6 Hz, 1H), 9.37 (t, 1H), 8.85-8.78 (m, 2H), 8.77-8.72 (m, 1H), 8.24-8.18 (m, 2H), 8.00-7.94 (m, 1H), 7.74-7.69 (m, 2H), 7.66-7.61 (m, 1H), 7.54 (s, 1H), 7.20-7.12 (m, 2H), 7.09 (s, 1H), 5.87-5.70 (m, 1H), 4.40 (d, J=5.9 Hz, 2H), 3.77 (s, 3H), 2.87-2.81 (m, 2H), 2.80-2.75 (m, 1H), 2.74-2.71 (m, 1H), 2.71-2.63 (m, 1H), 2.47 (s, 3H), 2.26-2.17 (m, 1H), 1.91 (s, 3H), 1.31 (t, J=7.6 Hz, 3H).

### Example B2. N-(3-(1-(2,6-Dioxopiperidin-3-yl)-1H-benzo[d]imidazol-6-yl)prop-2-yn-1-yl)-5-(8-(7-ethyl-1,3-dimethyl-2-oxo-1,2-dihydroquinolin-5-yl)isoquinolin-3-yl)picolinamide

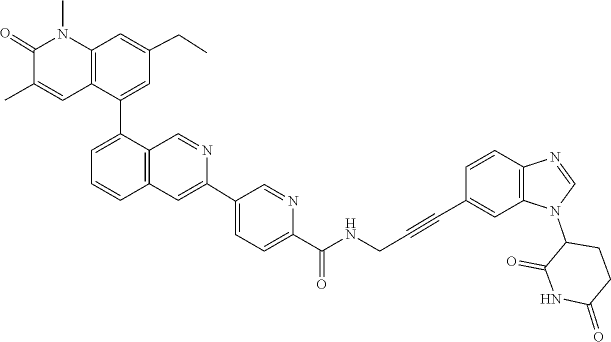 [SMILES: CCc1cc(-c2cccc3cc(-c4ccc(C(=O)NCC#Cc5ccc6ncn(C7CCC(=O)NC7=O)c6c5)nc4)ncc23)c2cc(C)c(=O)n(C)c2c1]

To a solution of 5-(8-(7-ethyl-1,3-dimethyl-2-oxo-1,2-dihydroquinolin-5-yl)isoquinolin-3-yl)picolinic acid (15 mg, 33.4 μmol, 1.0 eq.; Intermediate 4), HATU (16 mg, 43.4 μmol, 1.3 eq.), and DIPEA (0.1 mL, 100 μmol, 3.0 eq.) in DMF (4 mL) was added a mixture of 3-(6-(3-aminoprop-1-yn-1-yl)-1H-benzo[d]imidazol-1-yl)piperidine-2,6-dione and 3-(5-(3-aminoprop-1-yn-1-yl)-1H-benzo[d]imidazol-1-yl)piperidine-2,6-dione (20 mg, 53.4 μmol, 1.6 eq.; Example A15, Step C) at 0° C. The mixture was stirred at room temperature for 1 hour under N₂ atmosphere. The resulting solution was diluted with 20 mL of H₂O and extracted with EtOAc (20 mL×3). The combined organic layers were washed with brine, dried over Na₂SO₄, and concentrated under reduced pressure. The resulting solution was purified by Prep-HPLC (Triant C18 5 m column, 0-60% MeOH in H₂O with 0.1% FA) to afford N-(3-(1-(2,6-dioxopiperidin-3-yl)-1H-benzo[d]imidazol-6-yl)prop-2-yn-1-yl)-5-(8-(7-ethyl-1,3-dimethyl-2-oxo-1,2-dihydroquinolin-5-yl)isoquinolin-3-yl)picolinamide (3.3 mg, 14% yield) as a white solid.

LC-MS (ESI): mass calcd. for C₄₃H₃₅N₇O₄, 713.3; m/z found, 714.5 [M+H]⁺.

¹H NMR (400 MHz, DMSO-d₆) δ 11.24 (s, 1H), 9.46 (d, J=1.7 Hz, 1H), 9.37 (t, J=5.8 Hz, 1H), 8.83-8.80 (m, 2H), 8.78-8.70 (m, 2H), 8.24-8.19 (m, 2H), 7.98 (t, 1H), 7.86 (s, 1H), 7.73 (d, J=8.4 Hz, 1H), 7.65 (d, J=7.0 Hz, 1H), 7.55 (s, 1H), 7.39 (d, J=7.4 Hz, 1H), 7.18 (s, 1H), 7.09 (s, 1H), 5.81 (dd, J=12.2, 5.0 Hz, 1H), 4.40 (d, J=5.9 Hz, 2H), 3.77 (s, 3H), 2.88-2.79 (m, 4H), 2.75-2.68 (m, 1H), 2.32-2.25 (m, 1H), 1.92 (s, 3H), 1.31 (t, J=7.6 Hz, 3H).

### Example B3. N-(3-(1-(2,6-Dioxopiperidin-3-yl)-5-methyl-1H-indazol-6-yl)prop-2-yn-1-yl)-5-(8-(7-ethyl-1,3-dimethyl-2-oxo-1,2-dihydroquinolin-5-yl)isoquinolin-3-yl)picolinamide

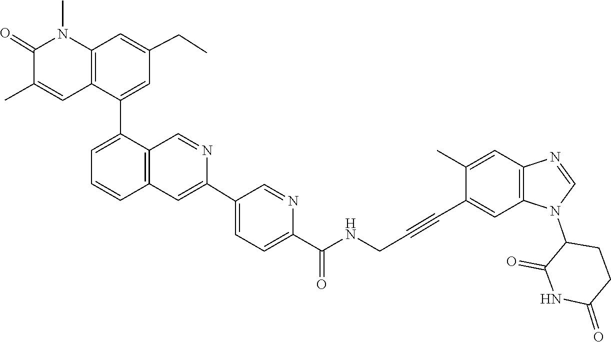 [SMILES: CCc1cc(-c2cccc3cc(-c4ccc(C(=O)NCC#Cc5cc6c(cc5C)ncn6C5CCC(=O)NC5=O)nc4)ncc23)c2cc(C)c(=O)n(C)c2c1]

N-(3-(1-(2,6-Dioxopiperidin-3-yl)-5-methyl-1H-indazol-6-yl)prop-2-yn-1-yl)-5-(8-(7-ethyl-1,3-dimethyl-2-oxo-1,2-dihydroquinolin-5-yl)isoquinolin-3-yl)picolinamide was prepared from 5-(8-(7-ethyl-1,3-dimethyl-2-oxo-1,2-dihydroquinolin-5-yl)isoquinolin-3-yl)picolinic acid (Intermediate 4) and 3-(6-(3-aminoprop-1-yn-1-yl)-5-methyl-1H-indazol-1-yl)piperidine-2,6-dione (as prepared in Example A8, Step C) according to the procedure described in Example A1, Step D. The title compound (6 mg, 12% yield) was obtained as a white solid.

LC-MS (ESI): mass calcd. for C₄₄H₃₇N₇O₄, 727.3; m/z found, 728 [M+H]⁺.

¹H NMR (400 MHz, DMSO-d₆) δ 11.06 (s, 1H), 9.47 (d, J=1.6 Hz, 1H), 9.40 (t, J=6.0 Hz, 1H), 8.85-8.80 (m, 2H), 8.78-8.74 (m, 1H), 8.24 (s, 1H), 8.21 (d, J=2.1 Hz, 1H), 8.05 (s, 1H), 8.01-7.95 (m, 1H), 7.77 (s, 1H), 7.67-7.63 (m, 2H), 7.55 (s, 1H), 7.19 (s, 1H), 7.10 (s, 1H), 5.85 (dd, J=11.6, 5.0 Hz, 1H), 4.44 (d, J=5.9 Hz, 2H), 3.77 (s, 3H), 2.87-2.83 (m, 2H), 2.78-2.67 (m, 3H), 2.46 (s, 3H), 2.27-2.21 (m, 1H), 1.92 (s, 3H), 1.32 (t, J=7.6 Hz, 3H).

### Example B4. N-(3-(3-((2,6-Dioxopiperidin-3-yl)methyl)phenyl)prop-2-yn-1-yl)-5-(8-(7-ethyl-1,3-dimethyl-2-oxo-1,2-dihydroquinolin-5-yl)isoquinolin-3-yl)picolinamide

### Step A: 3-(3-Bromobenzyl)piperidine-2,6-dione

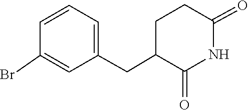 [SMILES: O=C1CCC(Cc2cccc(Br)c2)C(=O)N1]

To a solution of piperidine-2,6-dione (2 g, 17.7 mmol, 1.0 eq.) in THF (5 mL) was added LiHMDS (35.4 mL, 35.4 mmol, 2.0 eq., 1.0 M in THF) and 1-bromo-3-(bromomethyl)benzene (4.4 g, 17.7 mmol, 1.0 eq.) at −40° C. The mixture was stirred at 25° C. for 2 hours under N₂. The resulting solution was quenched with sat. aq. NH₄Cl and extracted with EtOAc (50 mL×3). The combined organic layers were washed with brine (50 mL), dried over Na₂SO₄, and concentrated under reduced pressure. The residue was purified by flash column chromatography on silica gel (100-200 mesh silica gel, 0-50% EtOAc in PE) to give 3-(3-bromobenzyl)piperidine-2,6-dione (500 mg, 10% yield) as a yellow solid.

LC-MS (ESI): mass calcd. for C₁₂H₁₂BrNO₂, 281.0/283.0; m/z found, 282/284 [M+H]⁺.

### Steps B-D: N-(3-(3-((2,6-Dioxopiperidin-3-yl)methyl)phenyl)prop-2-yn-1-yl)-5-(8-(7-ethyl-1,3-dimethyl-2-oxo-],2-dihydroquinolin-5-yl)isoquinolin-3-yl)picolinamide

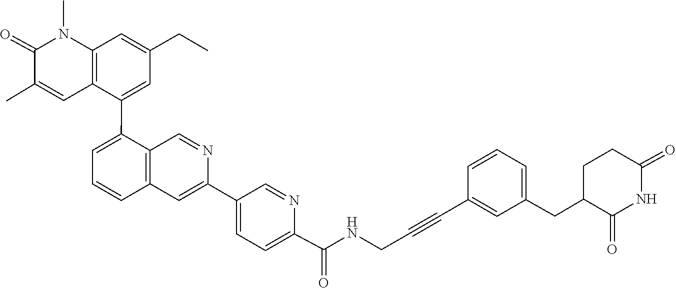 [SMILES: CCc1cc(-c2cccc3cc(-c4ccc(C(=O)NCC#Cc5cccc(CC6CCC(=O)NC6=O)c5)nc4)ncc23)c2cc(C)c(=O)n(C)c2c1]

N-(3-(3-((2,6-Dioxopiperidin-3-yl)methyl)phenyl)prop-2-yn-1-yl)-5-(8-(7-ethyl-1,3-dimethyl-2-oxo-1,2-dihydroquinolin-5-yl)isoquinolin-3-yl)picolinamide was prepared from 3-(3-bromobenzyl)piperidine-2,6-dione according to the procedures described in Example A1, Steps B D. The title compound (20 mg, 15% yield over 3 steps) was obtained as a white solid.

LC-MS (ESI): mass calcd. for C₄₃H₃₇N₅O₄, 687.3; m/z found, 688 [M+H]⁺.

¹H NMR (400 MHz, DMSO-d₆) δ 10.67 (s, 1H), 9.46 (d, J=2.1 Hz, 1H), 9.37-9.31 (m, 1H), 8.86-8.79 (m, 2H), 8.78-8.74 (m, 1H), 8.24-8.17 (m, 2H), 8.00-7.93 (m, 1H), 7.64 (d, J=7.1 Hz, 1H), 7.54 (s, 1H), 7.31-7.26 (m, 3H), 7.23 (d, J=6.5 Hz, 1H), 7.18 (s, 1H), 7.10 (s, 1H), 4.36 (d, J=6.0 Hz, 2H), 3.77 (s, 3H), 3.20-3.15 (m, 1H), 2.88-2.77 (m, 3H), 2.68-2.62 (m, 1H), 2.46-2.42 (m, 2H), 1.92 (s, 3H), 1.68-1.61 (m, 1H), 1.55-1.48 (m, 1H), 1.31 (t, J=7.6 Hz, 3H).

### Example B5. N-(3-(1-(2,6-Dioxopiperidin-3-yl)-1H-indazol-6-yl)prop-2-yn-1-yl)-5-(8-(7-ethyl-i,3-dimethyl-2-oxo-1,2-dihydroquinolin-5-yl)isoquinolin-3-yl)picolinamide

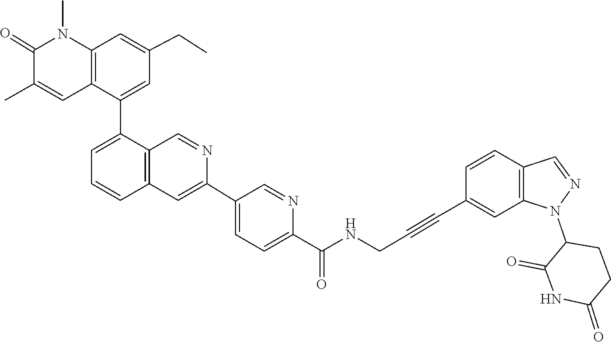 [SMILES: CCc1cc(-c2cccc3cc(-c4ccc(C(=O)NCC#Cc5ccc6cnn(C7CCC(=O)NC7=O)c6c5)nc4)ncc23)c2cc(C)c(=O)n(C)c2c1]

N-(3-(1-(2,6-Dioxopiperidin-3-yl)-1H-indazol-6-yl)prop-2-yn-1-yl)-5-(8-(7-ethyl-1,3-dimethyl-2-oxo-1,2-dihydroquinolin-5-yl)isoquinolin-3-yl)picolinamide was prepared from 5-(8-(7-ethyl-1,3-dimethyl-2-oxo-1,2-dihydroquinolin-5-yl)isoquinolin-3-yl)picolinic acid (Intermediate 4) and 3-(6-(3-aminoprop-1-yn-1-yl)-1H-indazol-1-yl)piperidine-2,6-dione (as prepared in Example A11, Step C) according to the procedure described in Example A1, Step D. The title compound (25.7 mg, 38% yield) was obtained as a white solid.

LC-MS (ESI): mass calcd. for C₄₃H₃₅N₇O₄, 713.3; m/z found, 714.4 [M+H]⁺.

¹H NMR (400 MHz, DMSO-d₆) δ 11.08 (s, 1H), 9.47 (d, J=2.0 Hz, 1H), 9.38 (t, J=5.8 Hz, 1H), 8.83-8.78 (m, 2H), 8.76 (dd, J=8.2, 2.1 Hz, 1H), 8.25-8.18 (m, 2H), 8.15 (s, 1H), 8.00-7.95 (m, 1H), 7.81 (s, 1H), 7.77 (d, J=8.4 Hz, 1H), 7.65 (d, J=7.0 Hz, 1H), 7.55 (s, 1H), 7.21-7.15 (m, 2H), 7.09 (s, 1H), 5.93-5.84 (m, 1H), 4.40 (d, J=5.7 Hz, 2H), 3.77 (s, 3H), 2.87-2.82 (m, 2H), 2.78-2.72 (m, 2H), 2.70-2.64 (m, 1H), 2.27-2.21 (m, 1H), 1.92 (s, 3H), 1.31 (t, J=7.6 Hz, 3H).

### Example B6. N-(3-(1-(2,6-Dioxopiperidin-3-yl)-4-methyl-1H-indazol-6-yl)prop-2-yn-1-yl)-5-(8-(7-ethyl-1,3-dimethyl-2-oxo-1,2-dihydroquinolin-5-yl)isoquinolin-3-yl)picolinamide

### Step A: 6-Bromo-4-methyl-4H-indazole

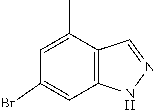

A mixture of 6-bromo-4-iodo-1H-indazole (2 g, 6.19 mmol, 1.0 eq.), 2,4,6-trimethyl-1,3,5,2,4,6-trioxatriborinane (7.8 g, 31.0 mmol, 5.0 eq., w.t. =50%), K₂CO₃ (2.6 g, 18.6 mmol, 3.0 eq.), and Pd(dppf)Cl₂ (453 mg, 619 μmol, 0.1 eq.) in 1,4-dioxane (30 mL) was stirred at 100° C. under N₂ for 16 hours. The resulting solution was concentrated in vacuo. The residue was purified by flash column chromatography on silica gel (100-200 mesh silica gel, 0-50% EtOAc in PE) to give 6-bromo-4-methyl-1H-indazole (300 mg, 23% yield) as a white solid.

LC-MS (ESI): mass calcd. for C₈H₇BrN₂, 210.0/212.0; m/z found, 210.9/212.9 [M+H]⁺.

### Step B: 3-(6-Bromo-4-methyl-4H-indazol-1-yl)piperidine-2,6-dione

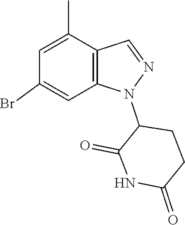 [SMILES: Cc1cc(Br)cc2c1cnn2C1CCC(=O)NC1=O]

To a mixture of 6-bromo-4-methyl-1H-indazole (300 mg, 1.42 mmol, 1 eq.) in DMF (10 mL) was added 3-bromopiperidine-2,6-dione (409 mg, 2.13 mmol, 1.5 eq.) and NaH (114 mg, 2.84 mmol, 2.0 eq., w.t. =60%) at 0° C. The mixture was stirred at 60° C. for 16 hours. The reaction mixture was partially concentrated in vacuo. The concentrate was quenched with sat. aq. NH₄Cl (20 mL) and extracted with EtOAc (30 mL×3). The combined organic layers were washed brine (20 mL), dried over Na₂SO₄, and concentrated in vacuo. The residue was purified by flash silica gel chromatography (0-70% EtOAc in PE) to afford 3-(6-bromo-4-methyl-1H-indazol-1-yl)piperidine-2,6-dione (110 mg, 24% yield) as a white solid.

LC-MS (ESI): mass calcd. for C₁₃H₁₂BrN₃O₂, 321.0/323.0; m/z found, 322/324 [M+H]⁺.

### Steps C-G: N-(3-(]-(2,6-Dioxopiperidin-3-yl)-4-methyl-1H-indazol-6-yl)prop-2-yn-1-yl)-5-(8-(7-ethyl-],3-dimethyl-2-oxo-],2-dihydroquinolin-5-yl)isoquinolin-3-yl)picolinamide

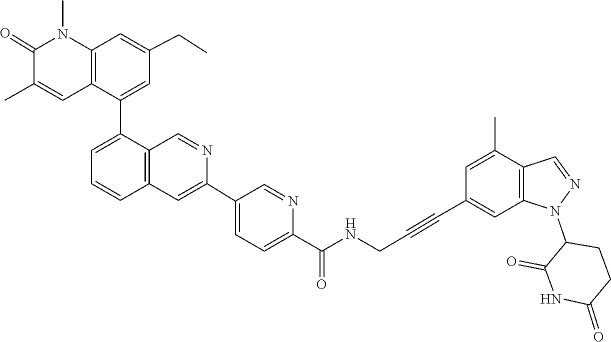 [SMILES: CCc1cc(-c2cccc3cc(-c4ccc(C(=O)NCC#Cc5cc(C)c6cnn(C7CCC(=O)NC7=O)c6c5)nc4)ncc23)c2cc(C)c(=O)n(C)c2c1]

N-(3-(1-(2,6-Dioxopiperidin-3-yl)-4-methyl-1H-indazol-6-yl)prop-2-yn-1-yl)-5-(8-(7-ethyl-1,3-dimethyl-2-oxo-1,2-dihydroquinolin-5-yl)isoquinolin-3-yl)picolinamide was prepared from 3-(6-bromo-4-methyl-1H-indazol-1-yl)piperidine-2,6-dione according to the procedures described in Example A5, Steps D-H. In the penultimate step, 5-(8-(7-ethyl-1,3-dimethyl-2-oxo-1,2-dihydroquinolin-5-yl)isoquinolin-3-yl)picolinic acid (Intermediate 4) was used as the coupling partner. The title compound (11 mg, 20% yield over 5 steps) was obtained as a white solid.

LC-MS (ESI): mass calcd. for C₄₄H₃₇N₇O₄, 727.3; m/z found, 728.5 [M+H]⁺.

¹H NMR (400 MHz, DMSO-d₆) δ 11.07 (s, 1H), 9.48-9.45 (m, 1H), 9.39-9.34 (m, 1H), 8.82 (s, 2H), 8.78-8.74 (m, 1H), 8.24-8.17 (m, 3H), 8.00-7.95 (m, 1H), 7.66-7.63 (m, 1H), 7.61 (s, 1H), 7.55 (s, 1H), 7.20-7.17 (m, 1H), 7.10 (s, 1H), 6.98 (s, 1H), 5.85 (d, J=11.5 Hz, 1H), 4.39 (d, J=5.8 Hz, 2H), 3.77 (s, 3H), 2.87-2.68 (m, 5H), 2.53 (s, 3H), 2.34-2.21 (m, 1H), 1.92 (s, 3H), 1.31 (t, J=7.6 Hz, 3H).

### Example B7. N-(3-(3-((2,6-Dioxopiperidin-3-yl)amino)-1-methyl-1H-pyrazol-5-yl)prop-2-yn-1-yl)-5-(8-(7-ethyl-1,3-dimethyl-2-oxo-1,2-dihydroquinolin-5-yl)isoquinolin-3-yl)picolinamide

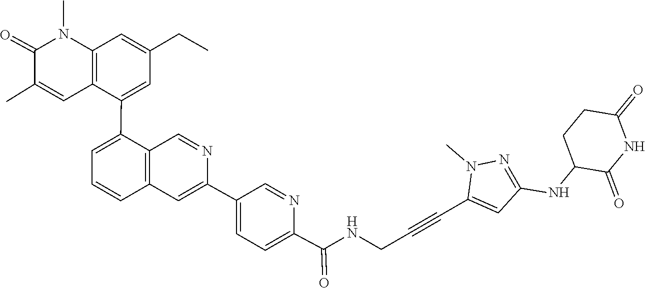 [SMILES: CCc1cc(-c2cccc3cc(-c4ccc(C(=O)NCC#Cc5cc(NC6CCC(=O)NC6=O)nn5C)nc4)ncc23)c2cc(C)c(=O)n(C)c2c1]

N-(3-(3-((2,6-Dioxopiperidin-3-yl)amino)-1-methyl-1H-pyrazol-5-yl)prop-2-yn-1-yl)-5-(8-(7-ethyl-1,3-dimethyl-2-oxo-1,2-dihydroquinolin-5-yl)isoquinolin-3-yl)picolinamide was prepared from 5-(8-(7-ethyl-1,3-dimethyl-2-oxo-1,2-dihydroquinolin-5-yl)isoquinolin-3-yl)picolinic acid (Intermediate 4) and 3-((5-(3-aminoprop-1-yn-1-yl)-1-methyl-1H-pyrazol-3-yl)amino)piperidine-2,6-dione (as prepared in Example A24, Step B) according to the procedures described in Example A1, Step D. The title compound (30.3 mg, 66% yield) was obtained as a white solid.

LC-MS (ESI): mass calcd. for C₄₀H₃₆N₈O₄, 692.3; m/z found, 693.3 [M+H]⁺.

¹H NMR (400 MHz, DMSO-d₆) δ 10.74 (s, 1H), 9.46 (s, 1H), 9.40 (t, J=5.9 Hz, 1H), 8.84-8.78 (m, 2H), 8.75 (d, J=8.2 Hz, 1H), 8.24-8.17 (m, 2H), 7.98 (t, J=7.6 Hz, 1H), 7.64 (d, J=7.0 Hz, 1H), 7.54 (s, 1H), 7.18 (s, 1H), 7.09 (s, 1H), 5.76 (s, 1H), 5.64 (d, J=6.8 Hz, 1H), 4.39 (d, J=5.6 Hz, 2H), 4.21-4.13 (m, 1H), 3.77 (s, 3H), 3.63 (s, 3H), 2.84 (q, J=7.3 Hz, 2H), 2.70-2.63 (m, 1H), 2.56-2.53 (m, 1H), 2.22-2.14 (m, 1H), 1.92 (s, 3H), 1.89-1.82 (m, 1H), 1.31 (t, J=7.6 Hz, 3H).

### Example B8. N-(3-(3-(2,6-Dioxopiperidin-3-yl)-1-methyl-2-oxo-2,3-dihydro-1H-benzo[d]imidazol-5-yl)prop-2-yn-1-yl)-5-(8-(7-ethyl-1,3-dimethyl-2-oxo-1,2-dihydroquinolin-5-yl)isoquinolin-3-yl)picolinamide

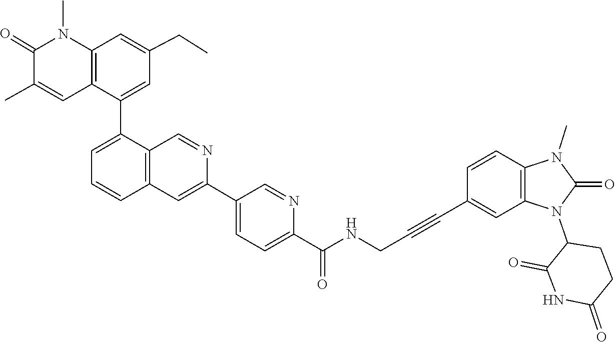 [SMILES: CCc1cc(-c2cccc3cc(-c4ccc(C(=O)NCC#Cc5ccc6c(c5)n(C5CCC(=O)NC5=O)c(=O)n6C)nc4)ncc23)c2cc(C)c(=O)n(C)c2c1]

N-(3-(3-(2,6-Dioxopiperidin-3-yl)-1-methyl-2-oxo-2,3-dihydro-1H-benzo[d]imidazol-5-yl)prop-2-yn-1-yl)-5-(8-(7-ethyl-1,3-dimethyl-2-oxo-1,2-dihydroquinolin-5-yl)isoquinolin-3-yl)picolinamide was prepared from 5-(8-(7-ethyl-1,3-dimethyl-2-oxo-1,2-dihydroquinolin-5-yl)isoquinolin-3-yl)picolinic acid (Intermediate 4) and 3-(6-(3-aminoprop-1-yn-1-yl)-3-methyl-2-oxo-2,3-dihydro-1H-benzo[d]imidazol-1-yl)piperidine-2,6-dione (as prepared in Example A18, Step C) according to the procedures described in Example A1, Step D. The title compound (60 mg, 60% yield) was obtained as a white solid.

LC-MS (ESI): mass calcd. for C₄₄H₃₇N₇O₅, 743.3; m/z found, 744.5 [M+H]⁺.

¹H NMR (400 MHz, DMSO-d₆) δ 11.07 (s, 1H), 9.48-9.44 (m, 1H), 9.34-9.28 (m, 1H), 8.83-8.80 (m, 2H), 8.76 (dd, J=8.2, 2.2 Hz, 1H), 8.23-8.19 (m, 2H), 8.00-7.95 (m, 1H), 7.66-7.62 (m, 1H), 7.55 (s, 1H), 7.26-7.09 (m, 5H), 5.42-5.35 (m, 1H), 4.36 (d, J=5.9 Hz, 2H), 3.77 (s, 3H), 3.34 (s, 3H), 2.89-2.80 (m, 3H), 2.74-2.66 (m, 1H), 2.63-2.57 (m, 1H), 2.02-1.97 (m, 1H), 1.94-1.90 (m, 3H), 1.33-1.29 (m, 3H).

### Example B9. 5-(8-(7-(1,1-Difluoroethyl)-1,3-dimethyl-2-oxo-1,2,3,4-tetrahydroquinazolin-5-yl)isoquinolin-3-yl)-N-(3-(1-(2,6-dioxopiperidin-3-yl)-1H-indazol-6-yl)prop-2-yn-1-yl)picolinamide

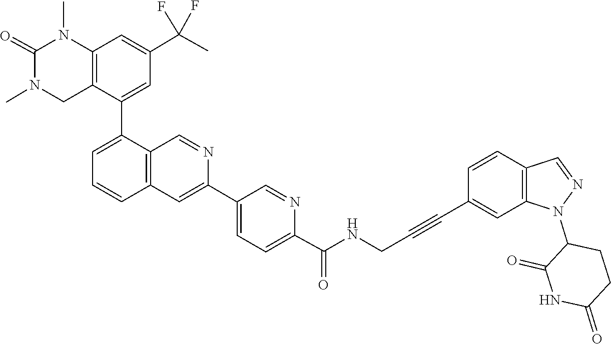 [SMILES: CN1Cc2c(-c3cccc4cc(-c5ccc(C(=O)NCC#Cc6ccc7cnn(C8CCC(=O)NC8=O)c7c6)nc5)ncc34)cc(C(C)(F)F)cc2N(C)C1=O]

5-(8-(7-(1,1-Difluoroethyl)-1,3-dimethyl-2-oxo-1,2,3,4-tetrahydroquinazolin-5-yl)isoquinolin-3-yl)-N-(3-(1-(2,6-dioxopiperidin-3-yl)-1H-indazol-6-yl)prop-2-yn-1-yl)picolinamide was prepared from 5-bromo-7-(1,1-difluoroethyl)-1,3-dimethyl-3,4-dihydroquinazolin-2(1H)-one (Intermediate 7) according to the procedures described in Intermediate 4, Steps E & F, and Example A1, Step D. In the final step, 3-(6-(3-aminoprop-1-yn-1-yl)-1H-indazol-1-yl)piperidine-2,6-dione (as prepared in Example A11, Step C) was used as the coupling partner. The title compound (11.2 mg, 16% yield) was obtained as a yellow solid.

LC-MS (ESI): mass calcd. for C₄₂H₃₄F₂N₈O₄, 752.3; m/z found, 753 [M+H]⁺.

¹H NMR (400 MHz, DMSO-d₆) δ 11.07 (s, 1H), 9.49 (d, J=1.6 Hz, 1H), 9.39 (t, J=5.9 Hz, 1H), 8.93 (s, 1H), 8.81-8.76 (m, 2H), 8.23 (d, J=8.2 Hz, 1H), 8.19 (d, J=8.4 Hz, 1H), 8.15 (s, 1H), 7.98-7.92 (m, 1H), 7.82 (s, 1H), 7.78 (d, J=8.3 Hz, 1H), 7.63 (d, J=6.8 Hz, 1H), 7.22 (s, 1H), 7.18 (d, J=9.3 Hz, 2H), 5.94-5.86 (m, 1H), 4.41 (d, J=5.9 Hz, 2H), 4.17 (d, J=14.9 Hz, 1H), 3.93 (d, J=14.9 Hz, 1H), 3.35 (s, 3H), 2.79-2.72 (m, 2H), 2.70 (s, 3H), 2.28-2.22 (m, 1H), 2.11-2.00 (m, 4H).

### Example B10. N-(3-(3-((2,6-Dioxopiperidin-3-yl)amino)phenyl)prop-2-yn-1-yl)-5-((7-ethyl-1,3-dimethyl-2-oxo-1,2-dihydroquinolin-5-yl)sulfonyl)-5′-methyl-[2,3′-bipyridine]-6′-carboxamide

### Step A: Methyl 3-((6-chloropyridin-3-yl)thio)propanoate

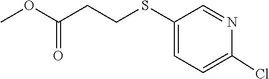

To a mixture of 2-chloro-5-iodopyridine (500 mg, 2.1 mmol, 1.0 eq.), Pd₂(dba)₃ (191 mg, 209 μmol, 0.1 eq.), methyl 3-mercaptopropanoate (753 mg, 6.26 mmol, 3.0 eq.), and Xant-Phos (242 mg, 418 μmol, 0.2 eq.) in 1,4-dioxane (10 mL) was added DIPEA (1 mL, 6.26 mmol, 3.0 eq.) at room temperature. The mixture was stirred for 85° C. for 3 hours. The resulting solution was diluted with 40 mL of H₂O and extracted with EtOAc (60 mL×3). The combined organic layers were washed with brine, dried over Na₂SO₄, and concentrated under reduced pressure. The residue was purified by flash column chromatography on silica gel (100-200 mesh silica gel, 0-60% EtOAc in PE) to give methyl 3-((6-chloropyridin-3-yl)thio)propanoate (300 mg, 62% yield) as a yellow oil.

LC-MS (ESI): mass calcd. for C₉H₁₀ClNO2S, 231.0; m/z found, 232 [M+H]⁺.

### Step B: Potassium 6-chloropyridine-3-thiolate

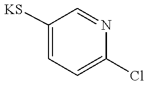

To a solution of methyl 3-((6-chloropyridin-3-yl)thio)propanoate (300 mg, 1.3 mmol, 1.0 eq.) in THF (5 mL) was added t-BuOK (290 mg, 2.5 mmol, 2.0 eq.) at −40° C. The mixture was stirred at −40° C. for 1 hour. The mixture was concentrated under reduced pressure to afford potassium 6-chloropyridine-3-thiolate (500 mg, crude) as a white solid.

LC-MS (ESI): mass calcd. for C₅H₄ClNS, 145.0; m/z found, 146 [M+H]⁺.

### Step C: 5-((6-Chloropyridin-3-yl)thio)-7-ethyl-],3-dimethylquinolin-2(H)-one

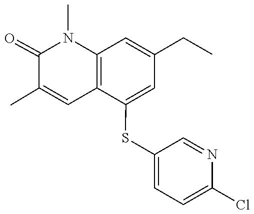 [SMILES: CCc1cc(Sc2ccc(Cl)nc2)c2cc(C)c(=O)n(C)c2c1]

To a mixture of potassium 6-chloropyridine-3-thiolate (500 mg, crude), Pd₂(dba)₃ (65 mg, 71.4 μmol, 0.1 eq.), 5-bromo-7-ethyl-1,3-dimethylquinolin-2(1H)-one (200 mg, 714 μmol, 1.0 eq.; Intermediate 8) and Xant-Phos (82 mg, 143 μmol, 0.2 eq.) in 1,4-dioxane (10 mL) was added DIPEA (373 μL, 2.1 mmol, 3.0 eq.) at room temperature. The mixture was stirred at 90° C. for 4 hours. The resulting solution was diluted with 40 mL of H₂O and extracted with EtOAc (60 mL×3). The combined organic layers were washed with brine, dried over Na₂SO₄, and concentrated under reduced pressure. The residue was purified by flash column chromatography on silica gel (100-200 mesh silica gel, 0-60% EtOAc in PE) to give 5-((6-chloropyridin-3-yl)thio)-7-ethyl-1,3-dimethylquinolin-2(1H)-one (80 mg, 33% yield) as an off-white solid.

LC-MS (ESI): mass calcd. for C₁₈H₁₇ClN₂OS, 344.1; m/z found, 345 [M+H]⁺.

### Step D: Methyl 5-((7-ethyl-1,3-dimethyl-2-oxo-1,2-dihydroquinolin-5-yl)thio)-5′-methyl-[2,3′-bipyridine]-6′-carboxylate

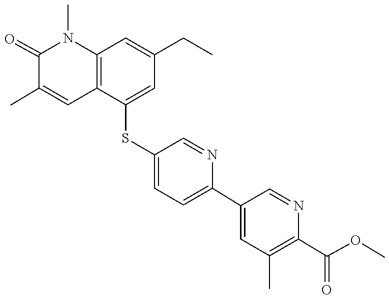 [SMILES: CCc1cc(Sc2ccc(-c3cnc(C(=O)OC)c(C)c3)nc2)c2cc(C)c(=O)n(C)c2c1]

To a mixture of 5-((6-chloropyridin-3-yl)thio)-7-ethyl-1,3-dimethylquinolin-2(1H)-one (70 mg, 203 μmol, 1.0 eq.), X-Phos Pd G2 (16 mg, 20.3 μmol, 0.1 eq.), and K₂CO₃ (56 mg, 406 μmol, 2.0 eq.) in 1,4-dioxane (2 mL) and water (0.1 mL) was added methyl 3-methyl-5-(4,4,5,5-tetramethyl-1,3,2-dioxaborolan-2-yl)picolinate (56 mg, 203 μmol, 1.0 eq.; Example A35, Step A) at room temperature. The mixture was stirred at 90° C. for 2 hours. The resulting solution was diluted with 30 mL of H₂O and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give methyl 5-((7-ethyl-1,3-dimethyl-2-oxo-1,2-dihydroquinolin-5-yl)thio)-5′-methyl-[2,3′-bipyridine]-6′-carboxylate (40 mg, 43% yield) as a solid.

LC-MS (ESI): mass calcd. for C₂₆H₂₅N₃O₃S, 459.2; m/z found, 460.1 [M+H]⁺.

### Step E: Methyl 5-((7-ethyl-],3-dimethyl-2-oxo-],2-dihydroquinolin-5-yl)sulfonyl)-5′-methyl-[2,3′-bipyridine]-6′-carboxylate

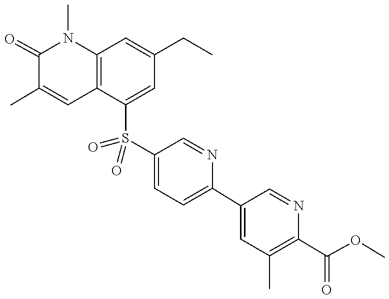 [SMILES: CCc1cc(S(=O)(=O)c2ccc(-c3cnc(C(=O)OC)c(C)c3)nc2)c2cc(C)c(=O)n(C)c2c1]

To a solution of methyl 5-((7-ethyl-1,3-dimethyl-2-oxo-1,2-dihydroquinolin-5-yl)thio)-5′- methyl-[2,3′-bipyridine]-6′-carboxylate (40 mg, 87.0 μmol, 1.0 eq.) in DCM (5 mL) was added m-CPBA (75 mg, 435 μmol, 5.0 eq.) at room temperature. The mixture was stirred for 5 hours at room temperature. The resulting solution was diluted with 30 mL of H₂O and extracted with EtOAc (50 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to give methyl 5-((7-ethyl-1,3-dimethyl-2-oxo-1,2-dihydroquinolin-5-yl)sulfonyl)-5′-methyl-[2,3′-bipyridine]-6′-carboxylate (12 mg, 28% yield) as a yellow solid.

LC-MS (ESI): mass calcd. for C₂₆H₂₅N₃₀₅S, 491.2; m/z found, 492.1 [M+H]⁺.

### Step F: 5-((7-Ethy-1,3-dimethyl-2-oxo-1,2-dihydroquinolin-5-yl)sulfonyl)-5′-methyl-[2,3′-bipyridine]-6′-carboxylic acid

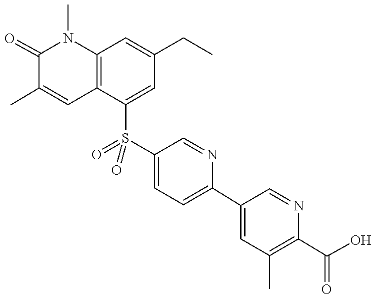 [SMILES: CCc1cc(S(=O)(=O)c2ccc(-c3cnc(C(=O)O)c(C)c3)nc2)c2cc(C)c(=O)n(C)c2c1]

To a solution of methyl 5-((7-ethyl-1,3-dimethyl-2-oxo-1,2-dihydroquinolin-5-yl)sulfonyl)-5′-methyl-[2,3′-bipyridine]-6′-carboxylate (10 mg, 21.8 μmol, 1.0 eq.) in THF (1 mL) and water (1 mL) was added LiGH (2 mg, 43.5 μmol, 2.0 eq.) at room temperature. The mixture was stirred at room temperature for 1 hour. The mixture was concentrated in vacuum to afford 5-((7-ethyl-1,3-dimethyl-2-oxo-1,2-dihydroquinolin-5-yl)sulfonyl)-5′-methyl-[2,3′-bipyridine]-6′-carboxylic acid (9 mg, 93% yield) as a yellow solid.

LC-MS (ESI): mass calcd. for C₂₅H₂₃N₃₀₅S, 477.1; m/z found, 478.1 [M+H]⁺.

### Step G: N-(3-(3-((2,6-Dioxopiperidin-3-yl)amino)phenyl)prop-2-yn-1-yl)-5-((7-ethyl-1,3-dimethyl-2-oxo-],2-dihydroquinolin-5-yl)sulfonyl)-5′-methyl-[2,3′-bipyridine]-6′-carboxamide

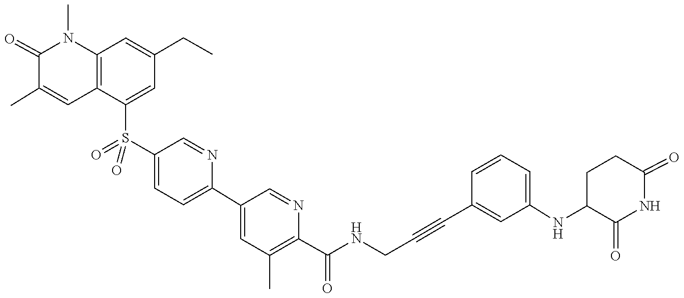 [SMILES: CCc1cc(S(=O)(=O)c2ccc(-c3cnc(C(=O)NCC#Cc4cccc(NC5CCC(=O)NC5=O)c4)c(C)c3)nc2)c2cc(C)c(=O)n(C)c2c1]

To a solution of 5-((7-ethyl-1,3-dimethyl-2-oxo-1,2-dihydroquinolin-5-yl)sulfonyl)-5′-methyl-[2,3′-bipyridine]-6′-carboxylic acid (10 mg, 201 μmol, 1.0 eq.), 3-((3-(3-aminoprop-1-yn-1-yl)phenyl)amino)piperidine-2,6-dione (8 mg, 31.4 μmol, 1.5 eq.; Intermediate 6) and HATU (16 mg, 41.9 μmol, 2.0 eq.) in DMF (5 mL) was added DIPEA (0.1 mL, 41.9 μmol, 2.0 eq.) at room temperature. The mixture was stirred at room temperature for 1 hour. The resulting solution was diluted with 30 mL of H₂O and extracted with EtOAc (30 mL×3). The combined organic layers were washed with brine, dried over Na₂SO₄, and concentrated under reduced pressure. The mixture was purified by Prep-HPLC (Triant C18 5 m column, 0-60% MeCN in H₂O with 0.1% FA) to afford N-(3-(3-((2,6-dioxopiperidin-3-yl)amino)phenyl)prop-2-yn-1-yl)-5-((7-ethyl-1,3-dimethyl-2-oxo-1,2-dihydroquinolin-5-yl)sulfonyl)-5′-methyl-[2,3′-bipyridine]-6′-carboxamide (4.1 mg, 26% yield) as a white solid.

LC-MS (ESI): mass calcd. for C₃₉H₃₆N₆₀₆S, 716.2; m/z found, 717.1 [M+H]⁺.

¹H NMR (400 MHz, DMSO-d₆) δ 10.77 (s, 1H), 9.33 (d, J=2.0 Hz, 1H), 9.18-9.11 (m, 2H), 8.59 (dd, J=8.5, 2.5 Hz, 1H), 8.48-8.44 (m, 2H), 8.35 (d, J=8.5 Hz, 1H), 8.06 (d, J=1.3 Hz, 1H), 7.77 (s, 1H), 7.11-6.99 (m, 1H), 6.71-6.60 (m, 3H), 6.00 (d, J=8.0 Hz, 1H), 4.41-4.33 (m, 1H), 4.29 (d, J=5.9 Hz, 2H), 3.67 (s, 3H), 2.90 (q, J=7.5 Hz, 2H), 2.78-2.69 (m, 1H), 2.63 (s, 3H), 2.59-2.53 (m, 1H), 2.19 (s, 3H), 2.10-2.04 (m, 1H), 1.92-1.81 (m, 1H), 1.32 (t, J=7.6 Hz, 3H).

### Example B11. 5-(2-Chloro-4-((7-ethyl-1,3-dimethyl-2-oxo-1,2-dihydroquinolin-5-yl)(methyl)amino)phenyl)-N-(3-(3-((2,6-dioxopiperidin-3-yl)amino)phenyl)prop-2-yn-1-yl)-3-methylpicolinamide

### Step A: tert-Butyl (4-bromo-3-chlorophenyl)carbamate

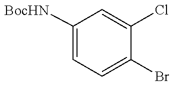 [SMILES: CC(C)(C)OC(=O)Nc1ccc(Br)c(Cl)c1]

To a solution of 4-bromo-3-chloroaniline (1 g, 4.84 mmol, 1.0 eq.) in DCM (20 mL) was added DMAP (59 mg, 484 μmol, 0.1 eq.) and Boc₂O(1.3 g, 5.81 mmol, 1.2 eq.) at 0° C. The mixture was stirred at room temperature for 3 hours. The reaction mixture was diluted with water (80 mL) and extracted with EtOAc (50 mL×3). The combined organic layers were washed brine (50 mL), dried over Na₂SO₄, and concentrated under reduced pressure. The residue was purified by flash silica gel chromatography (0-70% EtOAc in PE) to afford tert-butyl (4-bromo-3-chlorophenyl)carbamate (550 mg, 37% yield) as a yellow oil.

LC-MS (ESI): mass calcd. for C₁₁H₁₃BrClNO2, 305.0/307.0; no mass signal found.

### Step B: tert-Butyl (4-bromo-3-chlorophenyl)(methyl)carbamate

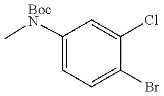 [SMILES: CN(C(=O)OC(C)(C)C)c1ccc(Br)c(Cl)c1]

To a solution of tert-butyl (4-bromo-3-chlorophenyl)carbamate (700 mg, 2.3 mmol, 1.0 eq.) in DMF (15 mL) was added NaH (110 mg, 4.6 mmol, 2.0 eq., w.t. =60%) at 0° C. After 30 mins, Mel (389 mg, 171 μL, 2.7 mmol, 1.2 eq.) was added to this mixture at 0° C. The mixture was stirred at room temperature for 16 hours. The reaction mixture was quenched with sat. aq. NH₄Cl (40 mL) and extracted with EtOAc (30 mL×3). The combined organic layers were washed brine (30 mL), dried over Na₂SO₄, and concentrated under reduced pressure. The residue was purified by flash silica gel chromatography (0-50%EtOAc in PE) to afford tert-butyl (4-bromo-3-chlorophenyl)(methyl)carbamate (650 mg, 89% yield) as a yellow oil.

LC-MS (ESI): mass calcd. for C₁₂H₁₅BrClNO2, 319.0/321.0; no mass signal found.

### Step C: Methyl 5-(4-((tert-butoxycarbonyl)(methyl)amino)-2-chlorophenyl)-3-methylpicolinate

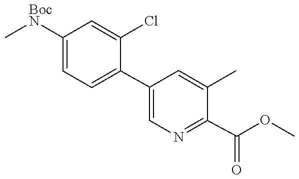

To a mixture of tert-butyl (4-bromo-3-chlorophenyl)(methyl)carbamate (500 mg, 1.6 mmol, 1.0 eq.) and methyl 3-methyl-5-(4,4,5,5-tetramethyl-1,3,2-dioxaborolan-2-yl)picolinate (562 mg, 2.0 mmol, 1.3 eq.; Example A35, Step A) in 1,4-dioxane (16 mL) and water (0.8 mL) was added Pd(dppf)Cl₂ (114.1 mg, 156.0 μmol, 0.1 eq.) and K₂CO₃ (647 mg, 4.7 mmol, 3.0 eq.) at room temperature. The mixture was stirred at 100° C. under N₂ for 3 hours. The reaction mixture was diluted with water (50 mL) and extracted with EtOAc (30 mL×3). The combined organic layers were washed brine (50 mL), dried over Na₂SO₄, and concentrated under reduced pressure.

The residue was purified by flash column chromatography on silica gel (100-200 mesh silica gel, 0-50% EtOAc in PE) to give methyl 5-(4-((tert-butoxycarbonyl)(methyl)amino)-2-chlorophenyl)-3-methylpicolinate (500 mg, 82% yield) as a yellow oil.

LC-MS (ESI): mass calcd. for C₂₀H₂₃ClN₂O₄, 390.1/392.1; m/z found, 391.1/393.1 [M+H]⁺.

### Step D: Methyl 5-(2-chloro-4-(methylamino)phenyl)-3-methylpicolinate

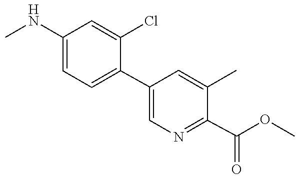

A sample of methyl 5-(4-((tert-butoxycarbonyl)(methyl)amino)-2-chlorophenyl)-3-methylpicolinate (200 mg, 512 μmol, 1.0 eq.) in 4M HCl in 1,4-dioxane (8 mL) was stirred at 0° C. The reaction mixture was stirred at room temperature for 1 hour. The reaction mixture was concentrated to afford methyl 5-(2-chloro-4-(methylamino)phenyl)-3-methylpicolinate (110 mg, 74% yield) as a yellow solid.

LC-MS (ESI): mass calcd. for C₁₅H₁₅ClN₂O₂, 290.1; m/z found, 291.1 [M+H]⁺.

### Step E: Methyl 5-(2-chloro-4-((7-ethyi-1,3-dimethyl-2-oxo-1,2-dihydroquinolin-5-yl)(methyl)amino)phenyl)-3-methylpicolinate

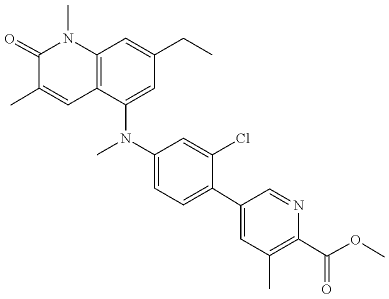 [SMILES: CCc1cc(N(C)c2ccc(-c3cnc(C(=O)OC)c(C)c3)c(Cl)c2)c2cc(C)c(=O)n(C)c2c1]

To a solution of methyl 5-(2-chloro-4-(methylamino)phenyl)-3-methylpicolinate (80 mg, 275 μmol, 1.0 eq.) and 5-bromo-7-ethyl-1,3-dimethylquinolin-2(1H)-one (93 mg, 330 μmol, 1.2 eq.; Intermediate 8) in toluene (8 mL) was added Pd₂(dba)₃ (25 mg, 27.5 μmol, 0.1 eq.), Xant-Phos (40 mg, 68.8 μmol, 0.25 eq.), and Cs₂CO₃ (179 mg, 550 μmol, 2.0 eq.) at room temperature. The mixture was stirred at 100° C. for 16 hours under N₂ atmosphere. The reaction mixture was diluted with water (40 mL) and extracted with EtOAc (30 mL×3). The combined organic layers were washed brine (40 mL), dried over Na₂SO₄, and concentrated under reduced pressure. The residue was purified by flash silica gel chromatography (0-10% MeOH in DCM) to afford methyl 5-(2-chloro-4-((7-ethyl-1,3-dimethyl-2-oxo-1,2-dihydroquinolin-5-yl)(methyl)amino)phenyl)-3-methylpicolinate (60 mg, 44% yield) as a yellow oil.

LC-MS (ESI): mass calcd. for C₂₈H₂₈ClN₃O₃, 489.2; m/z found, 490.2 [M+H]⁺.

### Step F: 5-(2-Chloro-4-((7-ethyl-1,3-dimethyl-2-oxo-1,2-dihydroquinolin-5-yl)(methyl)amino)phenyl)-3-methylpicolinic acid

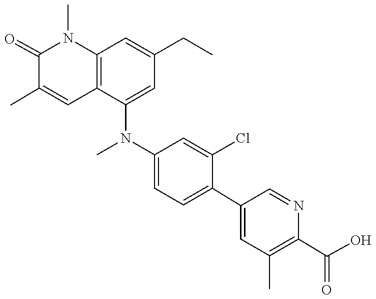 [SMILES: CCc1cc(N(C)c2ccc(-c3cnc(C(=O)O)c(C)c3)c(Cl)c2)c2cc(C)c(=O)n(C)c2c1]

To a solution of methyl 5-(2-chloro-4-((7-ethyl-1,3-dimethyl-2-oxo-1,2-dihydroquinolin-5-yl)(methyl)amino)phenyl)-3-methylpicolinate (40 mg, 81.6 μmol, 1.0 eq.) in THF (3 mL) and water (1.5 mL) was added LiOH (3 mg, 122 μmol, 1.5 eq.) at 0° C. The reaction mixture was stirred at room temperature for 1 hour. The reaction mixture was concentrated in vacuo to afford 5-(2-chloro-4-((7-ethyl-1,3-dimethyl-2-oxo-1,2-dihydroquinolin-5-yl)(methyl)amino)phenyl)-3-methylpicolinic acid (35 mg, 90% yield) as a white solid.

LC-MS (ESI): mass calcd. for C₂₇H₂₆ClN₃O₃, 475.2; m/z found, 476.3 [M+H]⁺.

### Step G: 5-(2-Chloro-4-((7-ethyl-],3-dimethyl-2-oxo-],2-dihydroquinolin-5-yl)(methyl)amino)phenyl)-N-(3-(3-((2,6-dioxopiperidin-3-yl)amino)phenyl)prop-2-yn-1-yl)-3-methylpicolinamide

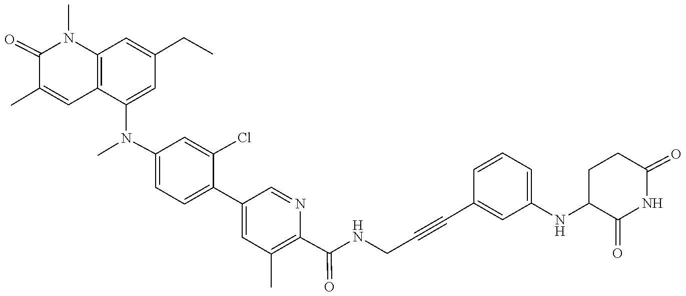 [SMILES: CCc1cc(N(C)c2ccc(-c3cnc(C(=O)NCC#Cc4cccc(NC5CCC(=O)NC5=O)c4)c(C)c3)c(Cl)c2)c2cc(C)c(=O)n(C)c2c1]

To a solution of 5-(2-chloro-4-((7-ethyl-1,3-dimethyl-2-oxo-1,2-dihydroquinolin-5-yl)(methyl)amino)phenyl)-3-methylpicolinic acid (35 mg, 73.5 μmol, 1.0 eq.) and 3-((3-(3-aminoprop-1-yn-1-yl)phenyl)amino)piperidine-2,6-dione (19 mg, 73.5 μmol, 1.0 eq.; Intermediate 6) in DMF (6 mL) was added HATU (56 mg, 147 μmol, 2.0 eq.) and DIPEA (64 μL, 368 μmol, 5.0 eq.) at room temperature. The mixture was stirred at room temperature for 1 hour. The reaction mixture was diluted with water (40 mL) and extracted with EtOAc (30 mL×3). The combined organic layers were washed brine (40 mL), and dried over Na₂SO₄, and concentrated under reduced pressure. The residue was purified by Prep-HPLC (Triant C18 5 m column, 0-60% MeCN in H₂O with 0.1% FA) to afford 5-(2-chloro-4-((7-ethyl-1,3-dimethyl-2-oxo-1,2-dihydroquinolin-5-yl)(methyl)amino)phenyl)-N-(3-(3-((2,6-dioxopiperidin-3-yl)amino)phenyl)prop-2-yn-1-yl)-3-methylpicolinamide (15.8 mg, 20% yield) as a white solid.

LC-MS (ESI): mass calcd. for C₄₁H₃₉ClN₆O₄, 714.3; m/z found, 715.3 [M+H]⁺.

¹H NMR (400 MHz, DMSO-d₆) δ 10.77 (s, 1H), 9.11-9.04 (m, 1H), 8.51-8.44 (m, 1H), 7.79-7.74 (m, 1H), 7.58 (s, 1H), 7.38 (s, 1H), 7.26 (d, J=8.6 Hz, 1H), 7.08-7.03 (m, 2H), 6.73-6.66 (m, 3H), 6.62 (d, J=7.5 Hz, 1H), 6.51-6.46 (m, 1H), 6.00 (d, J=7.9 Hz, 1H), 4.40-4.33 (m, 1H), 4.28 (d, J=5.8 Hz, 2H), 3.70 (s, 3H), 3.34 (s, 3H), 2.80-2.70 (m, 3H), 2.61-2.53 (m, 4H), 2.11-2.04 (m, 4H), 1.92-1.82 (m, 1H), 1.26 (t, J=7.6 Hz, 3H).

### Example B12. N-(3-(1-(2,6-Dioxopiperidin-3-yl)-1H-benzo[d]imidazol-5-yl)prop-2-yn-1-yl)-5-(8-(7-ethyl-1,3-dimethyl-2-oxo-1,2-dihydroquinolin-5-yl)isoquinolin-3-yl)picolinamide

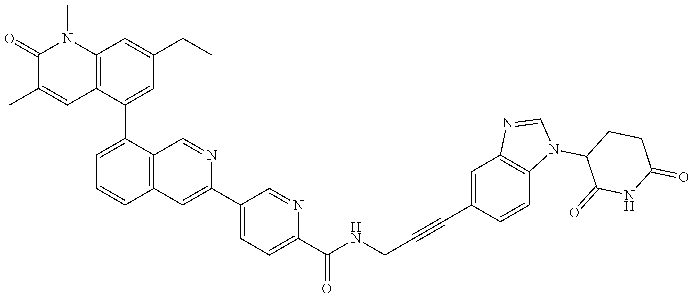 [SMILES: CCc1cc(-c2cccc3cc(-c4ccc(C(=O)NCC#Cc5ccc6c(c5)ncn6C5CCC(=O)NC5=O)nc4)ncc23)c2cc(C)c(=O)n(C)c2c1]

N-(3-(1-(2,6-Dioxopiperidin-3-yl)-1H-benzo[d]imidazol-5-yl)prop-2-yn-1-yl)-5-(8-(7-ethyl-1,3-dimethyl-2-oxo-1,2-dihydroquinolin-5-yl)isoquinolin-3-yl)picolinamide (3.2 mg, 13% yield) was obtained during the purification described in Example B2 as a white solid.

LC-MS (ESI): mass calcd. for C₄₃H₃₅N₇O₄, 713.3; m/z found, 714.5 [M+H]⁺.

¹H NMR (400 MHz, DMSO-d₆) δ 11.26 (s, 1H), 9.47 (d, J=1.6 Hz, 1H), 9.36 (t, J=6.0 Hz, 1H), 8.84-8.80 (m, 2H), 8.77 (dd, J=8.2, 2.2 Hz, 1H), 8.63 (s, 1H), 8.24-8.19 (m, 2H), 7.98 (t, 1H), 7.79 (s, 1H), 7.67-7.62 (m, 2H), 7.55 (s, 1H), 7.43-7.39 (m, 1H), 7.19 (s, 1H), 7.10 (s, 1H), 5.78 (dd, J=12.6, 4.7 Hz, 1H), 4.40 (d, J=5.8 Hz, 2H), 3.77 (s, 3H), 2.94-2.83 (m, 3H), 2.81-2.68 (m, 2H), 2.32-2.27 (m, 1H), 1.92 (s, 3H), 1.32 (t, J=7.6 Hz, 3H).

### Example B13. N-(3-(1-(2,6-Dioxopiperidin-3-yl)-1H-indazol-6-yl)prop-2-yn-1-yl)-5-(8-(7-ethyl-i,3-dimethyl-2-oxo-1,2,3,4-tetrahydropyrido[2,3-d]pyrimidin-5-yl)isoquinolin-3-yl)picolinamide

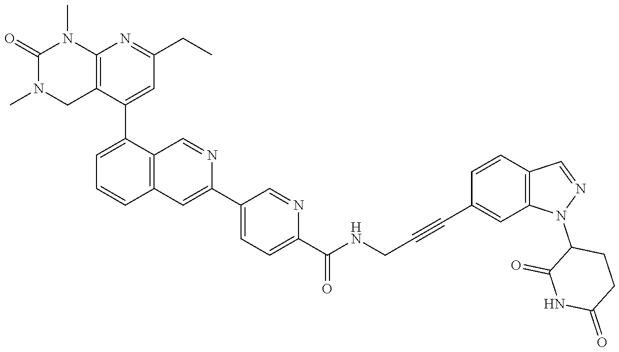 [SMILES: CCc1cc(-c2cccc3cc(-c4ccc(C(=O)NCC#Cc5ccc6cnn(C7CCC(=O)NC7=O)c6c5)nc4)ncc23)c2c(n1)N(C)C(=O)N(C)C2]

N-(3-(1-(2,6-Dioxopiperidin-3-yl)-1H-indazol-6-yl)prop-2-yn-1-yl)-5-(8-(7-ethyl-1,3-dimethyl-2-oxo-1,2,3,4-tetrahydropyrido[2,3-d]pyrimidin-5-yl)isoquinolin-3-yl)picolinamide was prepared from 5-chloro-7-ethyl-1,3-dimethyl-3,4-dihydropyrido[2,3-d]pyrimidin-2(1H)-one (Intermediate 9) according to the procedures described in Intermediate 4, Steps E & F, and Example A1, Step D. In the final step, 3-(6-(3-aminoprop-1-yn-1-yl)-1H-indazol-1-yl)piperidine-2,6-dione (as prepared in Example A11, Step C) was used as the coupling partner. The title compound (17 mg, 16% yield over 3 steps) was obtained as a white solid.

LC-MS (ESI): mass calcd. for C₄₁H₃₅N₉O₄, 717.3; m/z found, 718 [M+H]⁺.

¹H NMR (400 MHz, DMSO-d₆) δ 11.08 (s, 1H), 9.49 (d, J=1.8 Hz, 1H), 9.39 (t, J=6.0 Hz, 1H), 9.00 (s, 1H), 8.83-8.75 (m, 2H), 8.23 (d, J=8.3 Hz, 1H), 8.19 (d, J=8.3 Hz, 1H), 8.15 (s, 1H), 7.95 (t, 1H), 7.82 (s, 1H), 7.78 (d, J=8.4 Hz, 1H), 7.62 (d, J=7.1 Hz, 1H), 7.18 (d, J=8.4 Hz, 1H), 6.92 (s, 1H), 5.95-5.84 (m, 1H), 4.40 (d, J=5.9 Hz, 2H), 4.17 (d, J=14.8 Hz, 1H), 3.93 (d, J=14.8 Hz, 1H), 3.39 (s, 3H), 2.83-2.77 (m, 2H), 2.77-2.73 (m, 2H), 2.72 (s, 3H), 2.71-2.66 (m, 1H), 2.29-2.20 (m, 1H), 1.29 (t, J=7.5 Hz, 3H).

### Example B14. N-(3-(3-((2,6-Dioxopiperidin-3-yl)amino)phenyl)prop-2-yn-1-yl)-5-(4-((7-ethyl-i,3-dimethyl-2-oxo-1,2-dihydroquinolin-5-yl)(methyl)amino)-2-(trifluoromethyl)phenyl)-3-methylpicolinamide

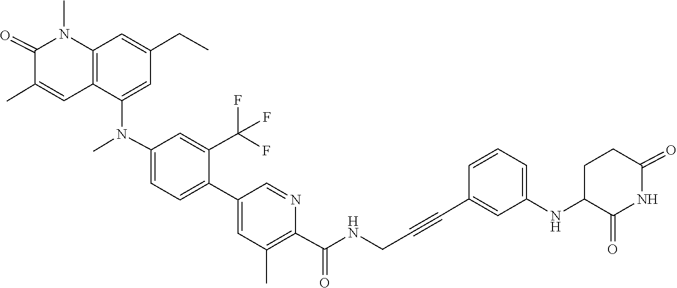 [SMILES: CCc1cc(N(C)c2ccc(-c3cnc(C(=O)NCC#Cc4cccc(NC5CCC(=O)NC5=O)c4)c(C)c3)c(C(F)(F)F)c2)c2cc(C)c(=O)n(C)c2c1]

N-(3-(3-((2,6-Dioxopiperidin-3-yl)amino)phenyl)prop-2-yn-1-yl)-5-(4-((7-ethyl-1,3-dimethyl-2-oxo-1,2-dihydroquinolin-5-yl)(methyl)amino)-2-(trifluoromethyl)phenyl)-3-methylpicolinamide was prepared from 4-bromo-3-(trifluoromethyl)aniline according to the procedures described in Example B11, Steps A-G. The title compound (13.2 mg, 2% yield over 7 steps) was obtained as a yellow solid.

LC-MS (ESI): mass calcd. for C₄₂H₃₉F₃N₆O₄, 748.3; m/z found, 749.3 [M+H]⁺.

¹H NMR (400 MHz, DMSO-d₆) δ 10.77 (s, 1H), 9.11 (t, J=5.7 Hz, 1H), 8.35 (s, 1H), 7.69-7.59 (m, 2H), 7.39 (s, 1H), 7.23 (d, J=8.6 Hz, 1H), 7.10-7.02 (m, 2H), 6.95 (s, 1H), 6.75-6.66 (m, 3H), 6.62 (d, J=7.4 Hz, 1H), 6.00 (d, J=7.9 Hz, 1H), 4.40-4.33 (m, 1H), 4.28 (d, J=5.8 Hz, 2H), 3.71 (s, 3H), 3.38 (s, 3H), 2.80-2.73 (m, 3H), 2.59 (s, 3H), 2.56-2.53 (m, 1H), 2.10 (s, 3H), 2.08-2.04 (m, 1H), 1.92-1.82 (m, 1H), 1.25 (t, J=7.5 Hz, 3H).

### Example B15. N-(3-(3-((2,6-Dioxopiperidin-3-yl)amino)phenyl)prop-2-yn-1-yl)-5-(4-((7-ethyl-i,3-dimethyl-2-oxo-1,2-dihydroquinolin-5-yl)(methyl)amino)-2-methylphenyl)-3-methylpicolinamide

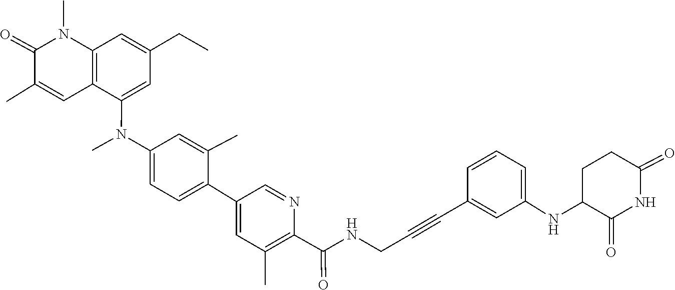 [SMILES: CCc1cc(N(C)c2ccc(-c3cnc(C(=O)NCC#Cc4cccc(NC5CCC(=O)NC5=O)c4)c(C)c3)c(C)c2)c2cc(C)c(=O)n(C)c2c1]

N-(3-(3-((2,6-Dioxopiperidin-3-yl)amino)phenyl)prop-2-yn-1-yl)-5-(4-((7-ethyl-1,3-dimethyl-2-oxo-1,2-dihydroquinolin-5-yl)(methyl)amino)-2-methylphenyl)-3-methylpicolinamide was prepared from 4-bromo-3-methylaniline according to the procedures described in Example B11, Steps A-G. The title compound (12.7 mg, 2% yield over 7 steps) was obtained as a yellow solid.

LC-MS (ESI): mass calcd. for C₄₂H₄₂N₆O₄, 694.3; m/z found, 695.5 [M+H]⁺.

¹H NMR (400 MHz, DMSO-d₆) δ 10.77 (s, 1H), 9.05 (s, 1H), 8.39 (d, J=1.6 Hz, 1H), 7.69 (d, J=1.4 Hz, 1H), 7.59 (s, 1H), 7.34 (s, 1H), 7.08-7.00 (m, 3H), 6.72-6.66 (m, 2H), 6.62 (d, J=7.5 Hz, 1H), 6.58 (d, J=2.1 Hz, 1H), 6.38 (dd, J=8.6, 2.3 Hz, 1H), 6.00 (d, J=7.9 Hz, 1H), 4.41-4.33 (m, 1H), 4.28 (d, J=5.9 Hz, 2H), 3.70 (s, 3H), 3.31 (s, 3H), 2.79-2.70 (m, 3H), 2.62-2.54 (m, 4H), 2.20 (s, 3H), 2.11-2.05 (m, 4H), 1.93-1.83 (m, 1H), 1.27-1.23 (m, 3H).

### Example B16. N-(3-(3-((2,6-Dioxopiperidin-3-yl)amino)phenyl)prop-2-yn-1-yl)-5-((7-ethyl-1,3-dimethyl-2-oxo-1,2-dihydroquinolin-5-yl)(methyl)amino)-5′-methyl-[2,3′-bipyridine]-6′-carboxamide

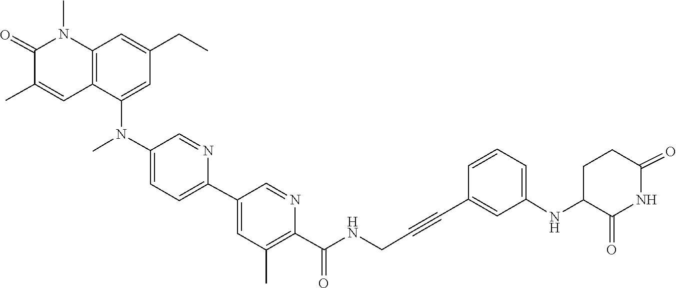 [SMILES: CCc1cc(N(C)c2ccc(-c3cnc(C(=O)NCC#Cc4cccc(NC5CCC(=O)NC5=O)c4)c(C)c3)nc2)c2cc(C)c(=O)n(C)c2c1]

N-(3-(3-((2,6-Dioxopiperidin-3-yl)amino)phenyl)prop-2-yn-1-yl)-5-((7-ethyl-1,3-dimethyl-2-oxo-1,2-dihydroquinolin-5-yl)(methyl)amino)-5′-methyl-[2,3′-bipyridine]-6′-carboxamide was prepared from methyl 5′-methyl-5-(methylamino)-[2,3′-bipyridine]-6′-carboxylate (Example A34, Step A) according to the procedures described in Example B11, Steps E-G. The title compound (16.1 mg, 26% yield over 3 steps) was obtained as a yellow solid.

LC-MS (ESI): mass calcd. for C₄₀H₃₉N₇O₄, 681.3; m/z found, 682.2 [M+H]⁺.

¹H NMR (400 MHz, DMSO-d₆) δ 10.77 (s, 1H), 9.08-9.00 (m, 2H), 8.25-8.22 (m, 1H), 8.03 (d, J=3.0 Hz, 1H), 7.94-7.89 (m, 1H), 7.59 (s, 1H), 7.39 (s, 1H), 7.10-7.03 (m, 2H), 7.01-6.97 (m, 1H), 6.72-6.66 (m, 2H), 6.64-6.60 (m, 1H), 6.00 (d, J=7.9 Hz, 1H), 4.41-4.33 (m, 1H), 4.28 (d, J=5.9 Hz, 2H), 3.71 (s, 3H), 3.39 (s, 3H), 2.79-2.71 (m, 3H), 2.63 (s, 3H), 2.59-2.53 (m, 1H), 2.10-2.05 (m, 4H), 1.92-1.81 (m, 1H), 1.28-1.24 (m, 3H).

### Example B17. N-(3-(3-((2,6-Dioxopiperidin-3-yl)amino)phenyl)prop-2-yn-1-yl)-5-((7-ethyl-1,3-dimethyl-2-oxo-1,2-dihydroquinolin-5-yl)oxy)-5′-methyl-[2,3′-bipyridine]-6′-carboxamide

### StepA: 5-((6-Chloropyridin-3-yl)oxy)-7-ethyl-],3-dimethylquinolin-2(H)-one

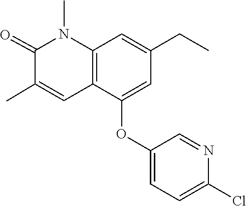 [SMILES: CCc1cc(Oc2ccc(Cl)nc2)c2cc(C)c(=O)n(C)c2c1]

A mixture of 7-ethyl-5-hydroxy-1,3-dimethylquinolin-2(1H)-one (50 mg, 230 μmol, 1.0 eq.; Intermediate 4, Step C), 2,2,6,6-tetramethylheptane-3,5-dione (51 mg, 276 μmol, 1.2 eq.), 2-chloro-5-iodopyridine (55 mg, 230 μmol, 1.0 eq.), CuI (22 mg, 115 μmol, 0.5 eq.), and Cs₂CO₃ (150 mg, 460 μmol, 2.0 eq.) in DMF (1 mL) was stirred at 100° C. for 16 hours under N₂. The resulting solution was diluted with 50 mL of water and extracted with EtOAc (50 mL×3). The combined organic layers were washed with brine (50 mL), dried over Na₂SO₄, and concentrated under reduced pressure. The residue was purified by flash column chromatography on silica gel (100-200 mesh silica gel, 0-30% EtOAc in PE) and Prep-HPLC to give 5-((6-chloropyridin-3-yl)oxy)-7-ethyl-1,3-dimethylquinolin-2(1H)-one (20 mg, 26% yield).

LC-MS (ESI): mass calcd. for C₁₈H₁₇ClN₂O₂, 328.1; m/z found, 329 [M+H]⁺.

### Step B: Methyl 5-((7-ethyl-1,3-dimethyl-2-oxo-1,2-dihydroquinolin-5-yl)oxy)-5′-methyl-[2,3′-bipyridine]-6′-carboxylate

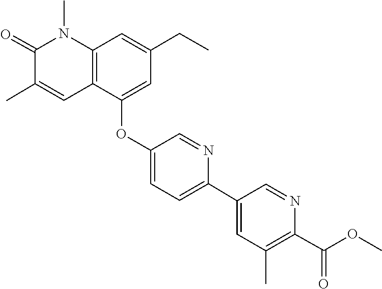 [SMILES: CCc1cc(Oc2ccc(-c3cnc(C(=O)OC)c(C)c3)nc2)c2cc(C)c(=O)n(C)c2c1]

A mixture of 5-((6-chloropyridin-3-yl)oxy)-7-ethyl-1,3-dimethylquinolin-2(1H)-one (20 mg, 60.8 μmol, 1.0 eq.), methyl 3-methyl-5-(4,4,5,5-tetramethyl-1,3,2-dioxaborolan-2-yl)picolinate (34 mg, 122 μmol, 2.0 eq.; Example A35, Step A), Pd(dppf)Cl₂ (5 mg, 6.08 μmol, 0.1 eq.), and K₃PO₄ (39 mg, 182 μmol, 3.0 eq.) in 1,4-dioxane (1 mL) and H₂O(0.1 mL) was stirred at 100° C. for 1 hour under N₂. The resulting solution was diluted with 50 mL of water and extracted with EtOAc (50 mL×3). The combined organic layers were washed with brine (50 mL), dried over Na₂SO₄, and concentrated under reduced pressure. The residue was purified by flash column chromatography on silica gel (100-200 mesh silica gel, 0-50% EtOAc in PE) to give methyl 5-((7-ethyl-1,3-dimethyl-2-oxo-1,2-dihydroquinolin-5-yl)oxy)-5′-methyl-[2,3′-bipyridine]-6′-carboxylate (20 mg, 74% yield) as a yellow solid.

LC-MS (ESI): mass calcd. for C₂₆H₂₅N₃O₄, 443.2; m/z found, 444 [M+H]⁺.

### Step C: 5-((7-Ethyl-1,3-dimethyl-2-oxo-1,2-dihydroquinolin-5-yl)oxy)-5′-methyl-[2,3′-bipyridine]-6′-carboxylic acid

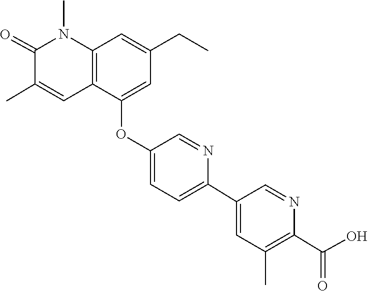 [SMILES: CCc1cc(Oc2ccc(-c3cnc(C(=O)O)c(C)c3)nc2)c2cc(C)c(=O)n(C)c2c1]

To a solution of methyl 5-((7-ethyl-1,3-dimethyl-2-oxo-1,2-dihydroquinolin-5-yl)oxy)-5′-methyl-[2,3′-bipyridine]-6′-carboxylate (20 mg, 45.1 μmol, 1.0 eq.) in THF (1 mL) and H₂O(1 mL) was added LiGH (2 mg, 90.2 μmol, 2.0 eq.). The mixture was stirred at 25° C. for 1 hour under N₂. The solvent was removed under reduced pressure to give 5-((7-ethyl-1,3-dimethyl-2-oxo-1,2-dihydroquinolin-5-yl)oxy)-5′-methyl-[2,3′-bipyridine]-6′-carboxylic acid (18.0 mg, crude, Li salt) as a yellow solid.

LC-MS (ESI): mass calcd. for C₂₅H₂₃N₃O₄, 429.2; m/z found, 430 [M+H]⁺.

### Step D: N-(3-(3-((2,6-Dioxopiperidin-3-yl)amino)phenyl)prop-2-yn-1-yl)-5-((7-ethyl-1,3-dimethyl-2-oxo-],2-dihydroquinolin-5-yl)oxy)-5′-methyl-[2,3′-bipyridine]-6′-carboxamide

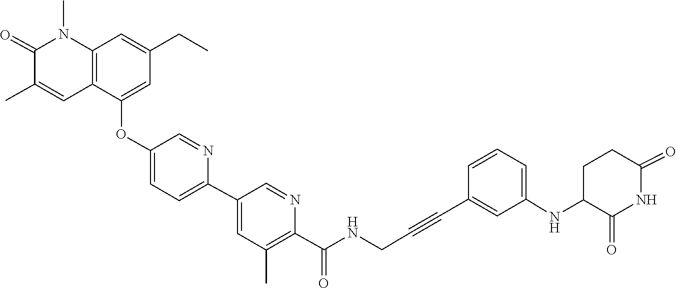 [SMILES: CCc1cc(Oc2ccc(-c3cnc(C(=O)NCC#Cc4cccc(NC5CCC(=O)NC5=O)c4)c(C)c3)nc2)c2cc(C)c(=O)n(C)c2c1]

A mixture of 5-((7-ethyl-1,3-dimethyl-2-oxo-1,2-dihydroquinolin-5-yl)oxy)-5′-methyl-[2,3′-bipyridine]-6′-carboxylic acid (10 mg, 23.3 μmol, 1 eq.), 3-((3-(3-aminoprop-1-yn-1-yl)phenyl)amino)piperidine-2,6-dione (9 mg, 34.9 μmol, 1.5 eq.; Intermediate 6), HATU (18 mg, 46.6 μmol, 2.0 eq.), and DIPEA (8.1 μL, 46.6 μmol, 2 eq.) in DMF (3 mL) was stirred at 0° C. for 30 mins under N₂. The resulting solution was diluted with 20 mL of water and extracted with EtOAc (20 mL×3). The combined organic layers were washed with brine (20 mL), dried over Na₂SO₄, and concentrated under reduced pressure. The residue was purified by Prep-HPLC to give N-(3-(3-((2,6-dioxopiperidin-3-yl)amino)phenyl)prop-2-yn-1-yl)-5-((7-ethyl-1,3-dimethyl-2-oxo-1,2-dihydroquinolin-5-yl)oxy)-5′-methyl-[2,3′-bipyridine]-6′-carboxamide (4.2 mg, 26% yield) as a yellow solid.

LC-MS (ESI): mass calcd. for C₃₉H₃₆N₆O₅, 668.3; m/z found, 669 [M+H]⁺.

¹H NMR (400 MHz, DMSO-d₆) δ 10.77 (s, 1H), 9.17-9.05 (m, 2H), 8.59 (d, J=2.9 Hz, 1H), 8.38 (d, J=1.5 Hz, 1H), 8.17 (d, J=8.7 Hz, 1H), 7.91 (s, 1H), 7.56 (dd, J=8.8, 2.9 Hz, 1H), 7.23 (s, 1H), 7.06 (t, J=7.9 Hz, 1H), 6.79 (s, 1H), 6.74-6.66 (m, 2H), 6.63 (d, J=7.5 Hz, 1H), 6.00 (s, 1H), 4.43-4.34 (m, 1H), 4.34-4.25 (m, 2H), 3.69 (s, 3H), 2.79-2.68 (m, 3H), 2.66 (s, 3H), 2.60-2.53 (m, 1H), 2.16-2.05 (m, 4H), 1.94-1.81 (m, 1H), 1.21 (t, J=7.6 Hz, 3H).

### Example B18. N-(3-(3-((2,6-Dioxopiperidin-3-yl)amino)phenyl)prop-2-yn-1-yl)-5-(4-((7-ethyl-1,3-dimethyl-2-oxo-1,2-dihydroquinolin-5-yl)(methyl)amino)phenyl)-3-methylpicolinamide

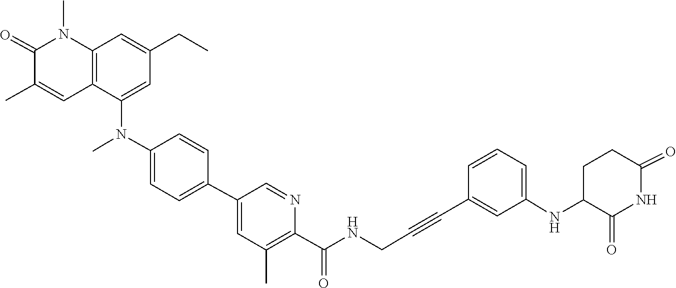 [SMILES: CCc1cc(N(C)c2ccc(-c3cnc(C(=O)NCC#Cc4cccc(NC5CCC(=O)NC5=O)c4)c(C)c3)cc2)c2cc(C)c(=O)n(C)c2c1]

N-(3-(3-((2,6-Dioxopiperidin-3-yl)amino)phenyl)prop-2-yn-1-yl)-5-(4-((7-ethyl-1,3-dimethyl-2-oxo-1,2-dihydroquinolin-5-yl)(methyl)amino)phenyl)-3-methylpicolinamide was prepared from 4-bromo-N-methylaniline according to the procedures described in Example B11, Steps C & E-G. The title compound (18.2 mg, 18% yield over 4 steps) was obtained as a yellow solid.

LC-MS (ESI): mass calcd. for C₄₁H₄N₆O₄, 680.3; m/z found, 681.2 [M+H]⁺.

¹H NMR (400 MHz, DMSO-d₆) δ 10.77 (s, 1H), 9.00 (t, J=5.9 Hz, 1H), 8.68 (d, J=1.9 Hz, 1H), 7.93 (d, J=1.7 Hz, 1H), 7.63 (d, J=8.9 Hz, 2H), 7.56 (s, 1H), 7.35 (s, 1H), 7.09-7.01 (m, 2H), 6.74-6.66 (m, 2H), 6.66-6.60 (m, 3H), 6.00 (d, J=7.9 Hz, 1H), 4.45-4.33 (m, 1H), 4.28 (d, J=5.9 Hz, 2H), 3.70 (s, 3H), 3.34 (s, 3H), 2.79-2.70 (m, 3H), 2.64-2.60 (m, 3H), 2.59-2.53 (m, 1H), 2.11-2.04 (m, 4H), 1.91-1.79 (m, 1H), 1.25 (d, J=7.7 Hz, 3H).

### Example B19. N-(3-(3-((2,6-Dioxopiperidin-3-yl)amino)phenyl)prop-2-yn-1-yl)-5-(4-((7-ethyl-1,3-dimethyl-2-oxo-1,2-dihydroquinolin-5-yl)(methyl)amino)-2-fluorophenyl)-3-methylpicolinamide

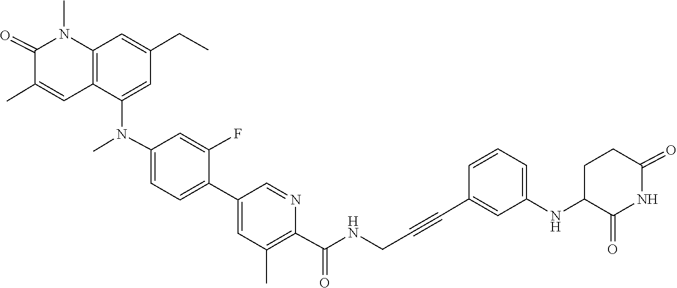 [SMILES: CCc1cc(N(C)c2ccc(-c3cnc(C(=O)NCC#Cc4cccc(NC5CCC(=O)NC5=O)c4)c(C)c3)c(F)c2)c2cc(C)c(=O)n(C)c2c1]

N-(3-(3-((2,6-Dioxopiperidin-3-yl)amino)phenyl)prop-2-yn-1-yl)-5-(4-((7-ethyl-1,3-dimethyl-2-oxo-1,2-dihydroquinolin-5-yl)(methyl)amino)-2-fluorophenyl)-3-methylpicolinamide was prepared from 4-bromo-3-fluoroaniline according to the procedures described in Example B11, Steps A-G. The title compound (11.2 mg, 2% yield over 7 steps) was obtained as a yellow solid.

LC-MS (ESI): mass calcd. for C₄₁H₃₉FN₆O₄, 698.3; m/z found, 699.4 [M+H]⁺.

¹H NMR (400 MHz, DMSO-d₆) δ 10.78 (s, 1H), 9.05 (t, J=5.8 Hz, 1H), 8.57 (s, 1H), 7.84 (s, 1H), 7.55 (s, 1H), 7.44-7.37 (m, 2H), 7.08-7.04 (m, 2H), 6.73-6.68 (m, 2H), 6.63 (d, J=7.5 Hz, 1H), 6.50 (d, J=14.5 Hz, 1H), 6.36 (d, J=8.6 Hz, 1H), 6.00 (d, J=7.8 Hz, 1H), 4.40-4.35 (m, 1H), 4.29 (d, J=5.7 Hz, 2H), 3.71 (s, 3H), 3.35 (s, 3H), 2.80-2.73 (m, 3H), 2.61 (s, 3H), 2.59-2.54 (m, 1H), 2.11-2.06 (m, 4H), 1.92-1.83 (m, 1H), 1.26 (t, J=7.5 Hz, 3H).

### Example B44. 3-(5-(3-(6-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)-1-oxoisoindolin-2-yl)prop-1-yn-1-yl)benzofuran-3-yl)piperidine-2,6-dion

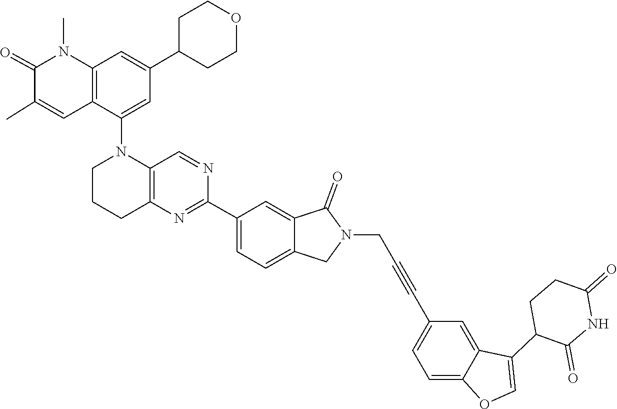 [SMILES: Cc1cc2c(N3CCCc4nc(-c5ccc6c(c5)C(=O)N(CC#Cc5ccc7occ(C8CCC(=O)NC8=O)c7c5)C6)ncc43)cc(C3CCOCC3)cc2n(C)c1=O]

### Step A: 2-(prop-2-yn-1-yl)-6-(4,4,5,5-tetramethyl-1,3,2-dioxaborolan-2-yl)isoindolin-1-one

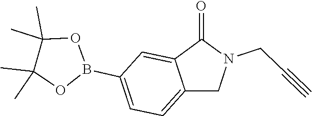 [SMILES: C#CCN1Cc2ccc(B3OC(C)(C)C(C)(C)O3)cc2C1=O]

To a solution of 6-(4,4,5,5-tetramethyl-1,3,2-dioxaborolan-2-yl)isoindolin-1-one (2 g, 7.7 mmol, 1.0 eq.) in DMF (30 mL) was added Cs₂CO₃ (7.5 g, 23.2 mmol, 3.0 eq.) and (3-bromoprop-1-yn-1-yl)trimethylsilane (2.2 g, 11.6 mmol, 1.5 eq.). Then the reaction was stirred at 80° C. for 3 hours. The reaction was monitored by LCMS. The resulting solution was diluted with 50 ml of water, then extracted with EA (3×200 mL) and washed with brine. The combined organic layer was dried over anhydrous sodium sulfate and concentrated under reduced pressure. The residue was purified on a silica gel chromatography (100-200 mesh silica gel, 0-80% EtOAc in PE) to give 2-(prop-2-yn-1-yl)-6-(4,4,5,5-tetramethyl-1,3,2-dioxaborolan-2-yl)isoindolin-1-one (450 mg, yield 20%) as a yellow solid.

LC-MS (ESI): mass calcd. for C₁₇H₂₀BNO₃, 297.2; m/z found, 298.2 [M+H]⁺.

### Step B: 3-(5-(3-(1-oxo-6-(4,4,5,5-tetramethyl-1,3,2-dioxaborolan-2-yl)isoindolin-2-yl)prop-1-yn-1-yl)benzofuran-3-yl)piperidine-2,6-dione

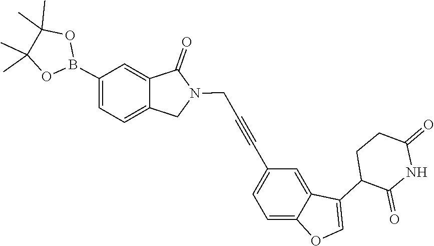 [SMILES: CC1(C)OB(c2ccc3c(c2)C(=O)N(CC#Cc2ccc4occ(C5CCC(=O)NC5=O)c4c2)C3)OC1(C)C]

To a solution of 2-(prop-2-yn-1-yl)-6-(4,4,5,5-tetramethyl-1,3,2-dioxaborolan-2-yl)isoindolin-1-one (200 mg, 673 μmol, 1.0 eq.) in DMF (5 mL) was added Pd(PPh₃)₂C₁₂ (47 mg, 67 μmol, 0.1 eq.), 3-(5-iodobenzofuran-3-yl)piperidine-2,6-dione (239 mg, 673 μmol, 1 eq.), CuI (26 mg, 135 μmol, 0.2 eq.) and TEA (2 mL). Then the reaction was stirred at 60° C. for 3 hours. The reaction was monitored by LCMS. The resulting solution was diluted with 50 ml of water, then extracted with EA (3×50 mL) and washed with brine. The combined organic layer was dried over anhydrous sodium sulfate and concentrated under reduced pressure. The residue was purified on a silica gel chromatography (100-200 mesh silica gel, 0-80% EtOAc in PE) to give 2-(prop-2-yn-1-yl)-6-(4,4,5,5-tetramethyl-1,3,2-dioxaborolan-2-yl)isoindolin-1-one (450 mg, yield 20%) as a yellow solid.

LC-MS (ESI): mass calcd. for C₃₀H₂₉BN₂O₆, 524.2; m/z found, 525.2 [M+H]⁺.

### Step C: 3-(5-(3-(6-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)-1-oxoisoindolin-2-yl)prop-1-yn-1-yl)benzofuran-3-yl)piperidine-2,6-dione

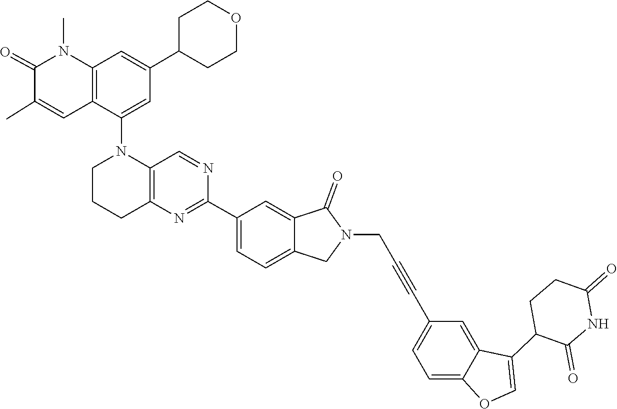 [SMILES: Cc1cc2c(N3CCCc4nc(-c5ccc6c(c5)C(=O)N(CC#Cc5ccc7occ(C8CCC(=O)NC8=O)c7c5)C6)ncc43)cc(C3CCOCC3)cc2n(C)c1=O]

To a solution of 3-(5-(3-(1-oxo-6-(4,4,5,5-tetramethyl-1,3,2-dioxaborolan-2-yl)isoindolin-2-yl)prop-1-yn-1-yl)benzofuran-3-yl)piperidine-2,6-dione (12 mg, 24 μmol, 1.0 eq.)in 1,4-Dioxane (3 mL) was added 5-(2-chloro-7,8-dihydropyrido[3,2-d]pyrimidin-5(6H)-yl)-1,3-dimethyl-7-(tetrahydro-2H-pyran-4-yl)quinolin-2(1H)-one (10 mg, 24 μmol, 1.0 eq.), Pd(dtbpf)Cl₂ (2 mg, 2 μmol, 0.1 eq.), CsF (11 mg, 71 μmol, 3.0 eq.) at room temperature. Then the reaction was stirred at 100° C. for 3 hours. The resulting solution was diluted with 20 mL of water and extracted with EtOAc (3×30 mL). The combined organic layers were washed with brine, dried over Na₂SO₄, and concentrated under reduced pressure. The residue was purified by flash prep-HPLC (Triant C18 5 m column, 0-60% MeCN in H₂O with 0.1% FA) to afford N-(3-(3-((2,6-dioxopiperidin-3-yl)amino)-2-fluorophenyl)prop-2-yn-1-yl)-5-(8-(7-ethyl-1,3-dimethyl-2-oxo-1,2-dihydroquinolin-5-yl)isoquinolin-3-yl)picolinamide (13 mg, 27% yield) as a yellow solid.

LC-MS (ESI): mass calcd. for C₄₇H₄₂N₆O₆, 786.3; m/z found, 787.4 [M+H]⁺.

¹H NMR (400 MHz, DMSO-d₆) δ 10.88 (s, 1H), 8.51 (s, 1H), 8.48-8.44 (m, 1H), 7.96 (s, 1H), 7.78-7.76 (m, 1H), 7.72 (s, 1H), 7.69 (d, J=8.1 Hz, 1H), 7.59 (d, J=8.5 Hz, 1H), 7.45-7.40 (m, 2H), 7.38 (s, 1H), 7.27 (s, 1H), 4.67 (d, J=2.7 Hz, 4H), 4.14 (dd, J=12.2, 4.8 Hz, 1H), 3.99-3.94 (m, 2H), 3.72 (s, 3H), 3.70-3.66 (m, 2H), 3.44-3.41 (m, 2H), 3.15-3.09 (m, 2H), 2.99-2.93 (m, 1H), 2.75-2.68 (m, 1H), 2.59-2.56 (m, 1H), 2.55-2.52 (m, 1H), 2.38-2.31 (m, 2H), 2.24-2.19 (m, 1H), 2.10 (s, 3H), 1.82-1.75 (m, 4H).

### Example B46. 5-(5-(6,8-dimethyl-7-oxo-2-(tetrahydro-2H-pyran-4-yl)-7,8-dihydro-1,8-naphthyridin-4-yl)-5,6,7,8-tetrahydropyrido[3,2-d]pyrimidin-2-yl)-N-(3-(4-(2,6-dioxopiperidin-3-yl)benzofuran-2-yl)prop-2-yn-1-yl)picolinamide

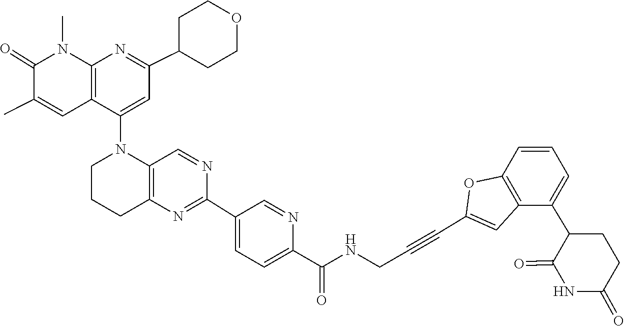 [SMILES: Cc1cc2c(N3CCCc4nc(-c5ccc(C(=O)NCC#Cc6cc7c(C8CCC(=O)NC8=O)cccc7o6)nc5)ncc43)cc(C3CCOCC3)nc2n(C)c1=O]

### Step A: tert-Butyl (4,6-dichloropyridin-2-yl)carbamate

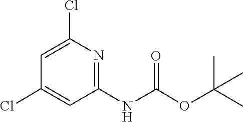 [SMILES: CC(C)(C)OC(=O)Nc1cc(Cl)cc(Cl)n1]

A mixture of 4,6-dichloropyridin-2-amine (13.1 g, 80 mmol, 1.0 eq.), Boc₂O(20.7 g, 96 mmol, 1.2 eq.), DMAP (488 mg, 4.0 mmol, 0.05 eq.), and TEA (22.2 mL, 160 mmol, 2.0 eq.) in DCM (130 mL) was stirred at 25° C. for 16 hours. The resulting solution was diluted with 300 mL of water and extracted with DCM (100 mL×3). The combined organic layers were washed with brine (100 mL), dried over Na₂SO₄, and concentrated under reduced pressure. The residue was purified by flash column chromatography on silica gel (100-200 mesh silica gel, 0-10% EtOAc in PE) to give tert-butyl (4,6-dichloropyridin-2-yl)carbamate (18 g, 77% yield) as a yellow solid. LC-MS (ESI): mass calcd. for C₁₀H₁₂Cl₂N₂O₂, 262.0; m/z found, 164.0 [M-56+H]+.

### Step B: tert-Butyl (4,6-dichloro-3-formylpyridin-2-yl)carbamate

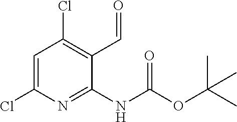 [SMILES: CC(C)(C)OC(=O)Nc1nc(Cl)cc(Cl)c1C=O]

To a solution of tert-butyl (4,6-dichloropyridin-2-yl)carbamate (14 g, 53.2 mmol, 1.0 eq.) in THF (100 mL) was added n-BuLi (53.2 mL, 133 mmol, 2.5 eq., 2.5 M in hexane) slowly at −65° C. under N₂. The mixture was stirred at this temperature for 30 mins. Then, DMF (12.3 mL, 159.6 mmol, 3.0 eq.) was added to the mixture at −65° C. The mixture was stirred at −65° C. for 2 hours under N₂. The resulting solution was quenched with 100 mL of aq. HCl (1 M in H₂O) and extracted with EtOAc (100 mL×3). The combined organic layers were washed with brine (100 mL), dried over Na₂SO₄, and concentrated under reduced pressure. The residue was purified by flash column chromatography on silica gel (100-200 mesh silica gel, 0-10% EtOAc in PE) to give tert-butyl (4,6-dichloro-3-formylpyridin-2-yl)carbamate (9.6 g, 62% yield) as a white solid. LC-MS (ESI): mass calcd. for C₁₁H₁₂Cl₂N₂O₃, 290.0; m/z found, 291.0 [M+H]⁺.

¹H NMR (400 MHz, DMSO-d₆) δ 10.64 (s, 1H), 10.11 (s, 1H), 7.63 (s, 1H), 1.46 (s, 9H).

### Step C: 2-Amino-4,6-dichloronicotinaldehyde

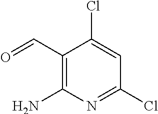 [SMILES: Nc1nc(Cl)cc(Cl)c1C=O]

To a solution of tert-butyl (4,6-dichloro-3-formylpyridin-2-yl)carbamate (2.6 g, 8.93 mmol, 1.0 eq.) in DCM (15 mL) was added HCl (10 mL, 4 M in dioxane). The mixture was stirred at 25° C. for 1 hour. The reaction mixture was then concentrated under reduced pressure.

The residue was diluted with sat. NaHCO₃ (60 mL) and extracted with EtOAc (50 mL×3). The combined organic layers were washed with brine (50 mL), dried over Na₂SO₄, and concentrated under reduced pressure to give 2-amino-4,6-dichloronicotinaldehyde (1.5 g, 88% yield) as a yellow solid. LC-MS (ESI): mass calcd. for C₆H₄C₁₂N₂O, 190.0; m/z found, 191.0 [M+H]⁺.

### Step D: 5,7-Dichloro-3-methyl-1,8-naphthyridin-2(1H)-one

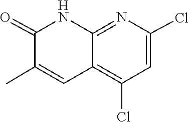

To a solution of 2-amino-4,6-dichloronicotinaldehyde (1.5 g, 7.8 mmol, 1.0 eq.), DMAP (958 mg, 7.8 mmol, 1.0 eq.), and TEA (3.2 mL, 23.4 mmol, 3.0 eq.) in DMF (20 mL) was added propionyl chloride (4.84 g, 2 eq., 52.4 mmol) slowly at 0° C. The mixture was warmed to 90° C. and stirred for 16 hours under N₂. The reaction mixture was then cooled and concentrated under reduced pressure. The residue was diluted with water (60 mL) and extracted with EtOAc (50 mL×3). The combined organic layers were washed with brine (50 mL), dried over Na₂SO₄, and concentrated under reduced pressure. The residue was purified by flash column chromatography on silica gel (100-200 mesh silica gel, 0-40% EtOAc in PE) to give 5,7-dichloro-3-methyl-1,8-naphthyridin-2(1H)-one (625 mg, 35% yield) as a yellow solid. LC-MS (ESI): mass calcd. for C₉H₆Cl₂N₂O, 228.0; m/z found, 229.0 [M+H]⁺. ¹H NMR (400 MHz, DMSO-d₆) δ 12.61 (s, 1H), 7.93 (d, J=1.4 Hz, 1H), 7.60 (s, 1H), 2.15 (d, J=1.3 Hz, 3H).

### Step E: 5,7-Dichloro-1,3-dimethyl-1,8-naphthyridin-2(1H)-one

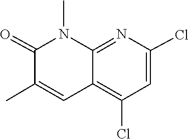

To a solution of 5,7-dichloro-3-methyl-1,8-naphthyridin-2(1H)-one (3.5 g, 15.3 mmol, 1.0 eq.) in DMF (35 mL) was added NaH (1.2 g, 30.6 mmol, 2.0 eq. wt=60%) at 0° C. The mixture was stirred at this temperature for 30 mins under N₂. Then, Mel (1.5 mL, 22.9 mmol, 1.5 eq.) was added to the mixture slowly at 0° C. The reaction mixture was stirred at 25° C. for 1 hour under N₂. The reaction was quenched with sat. NH₄Cl (100 mL) and extracted with EtOAc (100 mL×3). The combined organic layers were washed with brine (100 mL), dried over Na₂SO₄, and concentrated under reduced pressure. The residue was purified by flash column chromatography on silica gel (100-200 mesh silica gel, 0-40% EtOAc in PE) to give 5,7-dichloro-1,3-dimethyl-1,8-naphthyridin-2(1H)-one (3.3 g, 94% yield) as a yellow solid. LC-MS (ESI): mass calcd. for C₁₀H₈Cl₂N₂O, 242.0; m/z found, 243.0 [M+H]⁺. ¹H NMR (400 MHz, DMSO-d₆) δ 7.99 (d, J=1.5 Hz, 1H), 7.70 (s, 1H), 3.64 (s, 3H), 2.20 (d, J=1.2 Hz, 3H).

### Step F: 5-chloro-7-(3,6-dihydro-2H-pyran-4-yl)-1,3-dimethyl-1,8-naphthyridin-2(1H)-one

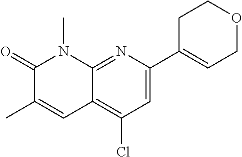 [SMILES: Cc1cc2c(Cl)cc(C3=CCOCC3)nc2n(C)c1=O]

To a solution of 5,7-dichloro-1,3-dimethyl-1,8-naphthyridin-2(1H)-one (500 mg, 2.06 mmol, 1.0 eq.) in 1,4-Dioxane (5 mL) and H₂O(1 mL) was added 2-(3,6-dihydro-2H-pyran-4-yl)-4,4,5,5-tetramethyl-1,3,2-dioxaborolane (454 mg, 2.16 mmol, 1.05 eq.), K₂CO₃ (853 mg, 6.17 mmol, 3.0 eq.) and Pd(dppf)Cl₂ (151 mg, 206 μmol, 0.1 eq.). The reaction mixture was stirred at 50° C. for 4 hours. The resulting solution was diluted with 20 mL of water and extracted with EtOAc (50 mL×3). The combined organic layers were washed with brine (50 mL), dried over Na₂SO₄ and concentrated under reduced pressure. The residue was purified by silica gel chromatography (100-200 mesh silica gel, 0-80% EtOAc in PE) to give 5-chloro-7-(3,6-dihydro-2H-pyran-4-yl)-1,3-dimethyl-1,8-naphthyridin-2(1H)-one (290 mg, 49% yield) as a yellow solid. LC-MS (ESI): mass calcd. for C₁₅H₁₅ClN₂O₂, 290.0; m/z found, 291 [M+H]⁺.

### Step G: 5-chloro-1,3-dimethyl-7-(tetrahydro-2H-pyran-4-yl)-1,8-naphthyridin-2(1H)-one

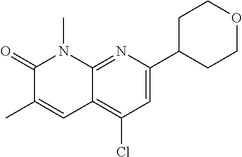 [SMILES: Cc1cc2c(Cl)cc(C3CCOCC3)nc2n(C)c1=O]

To a solution of 5-chloro-7-(3,6-dihydro-2H-pyran-4-yl)-1,3-dimethyl-1,8-naphthyridin-2(1H)-one (291 mg, 1.0 mmol, 1.0 eq.) in TFE (5 mL) was added Wilkinson's catalyst (277 mg, 0.3 mmol, 0.3 eq.) at 25° C. The mixture was stirred at 60° C. for 18 hours under H₂ atmosphere. The resulting solution was diluted with 50 ml of water and extracted with EtOAc (50 mL×3). The combined organic layers were washed with brine (20 mL), dried over anhydrous sodium sulfate and concentrated under reduced pressure. The residue was purified by flash chromatography (100-200 mesh silica gel, 0-80% EtOAc in PE) to give 5-chloro-1,3-dimethyl-7-(tetrahydro-2H-pyran-4-yl)-1,8-naphthyridin-2(1H)-one (218 mg, 75% yield) as a yellow solid. LC-MS (ESI): mass calcd. for C₁₅H₁₇ClN₂O₂, 292.0; m/z found, 293 [M+H]⁺.

### Step H: methyl 5-(5-(6,8-dimethyl-7-oxo-2-(tetrahydro-2H-pyran-4-yl)-7,8-dihydro-1,8-naphthyridin-4-yl)-5,6,7,8-tetrahydropyrido[3,2-d]pyrimidin-2-yl)picolinate

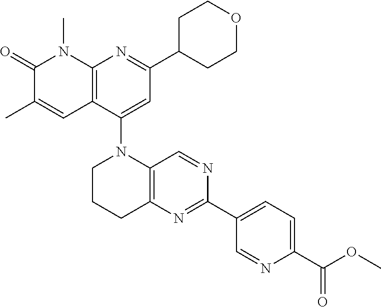

To a mixture of 5-chloro-1,3-dimethyl-7-(tetrahydro-2H-pyran-4-yl)-1,8-naphthyridin-2(1H)-one (80 mg, 273 μmol, 1.0 eq.), Ru-Phos Pd G1 (20 mg, 27.3 μmol, 1.0 eq.) and Ru-Phos ((13 mg, 27.3 μmol, 0.1 eq.)), Cs₂CO₃ (267 mg, 820 μmol, 3.0 eq.) in 1,4-Dioxane (5 mL) was added methyl 5-(5,6,7,8-tetrahydropyrido[3,2-d]pyrimidin-2-yl)picolinate (111 mg, 410 μmol, 1.5 eq.) at 25° C. The mixture was stirred at 100° C. for 2.5 hours under N₂ atmosphere. The resulting solution was diluted with 50 ml of water and extracted with EtOAc (20 mL×3). The combined organic layers were washed with brine (20 mL), dried over anhydrous sodium sulfate and concentrated under reduced pressure. The residue was purified by flash chromatography (100-200 mesh silica gel, 0-100% EtOAc in PE) to afford methyl 5-(5-(6,8-dimethyl-7-oxo-2-(tetrahydro-2H-pyran-4-yl)-7,8-dihydro-1,8-naphthyridin-4-yl)-5,6,7,8-tetrahydropyrido[3,2-d]pyrimidin-2-yl)picolinate (80 mg, 56% yield) as a yellow solid. LC-MS (ESI): mass calcd. for C₂₉H₃₀N₆O₄, 526.2; m/z found, 527 [M+H]⁺.

### Step I: lithium 5-(5-(6,8-dimethyl-7-oxo-2-(tetrahydro-2H-pyran-4-yl)-7,8-dihydro-1,8-naphthyridin-4-yl)-5,6,7,8-tetrahydropyrido[3,2-d]pyrimidin-2-yl)picolinate

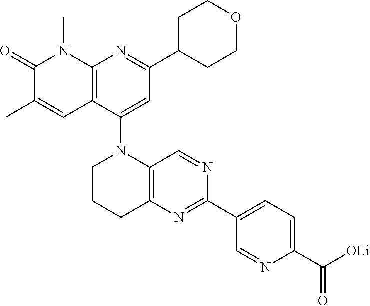

To a solution of methyl 5-(5-(6,8-dimethyl-7-oxo-2-(tetrahydro-2H-pyran-4-yl)-7,8-dihydro-1,8-naphthyridin-4-yl)-5,6,7,8-tetrahydropyrido[3,2-d]pyrimidin-2-yl)picolinate (80 mg, 152 μmol, 1.0 eq.) in THF (2 mL) and water (1 mL) was added LiGH (13 mg, 304 μmol, 2.0 eq.) at 25° C. The mixture was stirred at 25° C. for 1 hour. The reaction was concentrated under reduced pressure to afford lithium 5-(5-(6,8-dimethyl-7-oxo-2-(tetrahydro-2H-pyran-4-yl)-7,8-dihydro-1,8-naphthyridin-4-yl)-5,6,7,8-tetrahydropyrido[3,2-d]pyrimidin-2-yl)picolinate (80 mg, crude) as a yellow solid. LC-MS (ESI): mass calcd. for C₂₈H₂₇LiN₆O₄, 518.2; m/z found, 513 [M+H]⁺.

### Step J: 5-(5-(6,8-dimethyl-7-oxo-2-(tetrahydro-2H-pyran-4-yl)-7,8-dihydro-1,8-naphthyridin-4-yl)-5,6,7,8-tetrahydropyrido[3,2-d]pyrimidin-2-yl)-N-(3-(4-(2,6-dioxopiperidin-3-yl)benzofuran-2-yl)prop-2-yn-1-yl)picolinamide

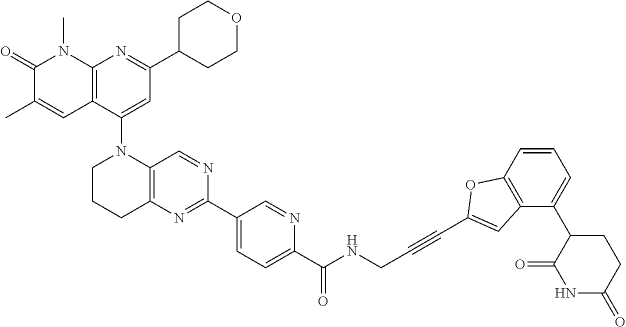 [SMILES: Cc1cc2c(N3CCCc4nc(-c5ccc(C(=O)NCC#Cc6cc7c(C8CCC(=O)NC8=O)cccc7o6)nc5)ncc43)cc(C3CCOCC3)nc2n(C)c1=O]

To a solution of lithium 5-(5-(6,8-dimethyl-7-oxo-2-(tetrahydro-2H-pyran-4-yl)-7,8-dihydro-1,8-naphthyridin-4-yl)-5,6,7,8-tetrahydropyrido[3,2-d]pyrimidin-2-yl)picolinate (40 mg, 78.0 μmol, 1.0 eq.), HATU (60 mg, 156 μmol, 2.0 eq.) and 3-(2-(3-aminoprop-1-yn-1-yl)benzofuran-4-yl)piperidine-2,6-dione (29 mg, 101 μmol, 1.3 eq.) in DMF (3 mL) was added DIEA (41 μL, 234 μmol, 3.0 eq.) at room temperature. The mixture was stirred at 25° C. for 20 mins under N₂ atmosphere. The resulting solution was diluted with 10 ml of water and extracted with EtOAc (10 mL×3). The combined organic layers were washed with brine (10 mL), dried over anhydrous sodium sulfate and concentrated under reduced pressure. The residue was purified by Prep-HPLC to afford 5-(5-(6,8-dimethyl-7-oxo-2-(tetrahydro-2H-pyran-4-yl)-7,8-dihydro-1,8-naphthyridin-4-yl)-5,6,7,8-tetrahydropyrido[3,2-d]pyrimidin-2-yl)-N-(3-(4-(2,6-dioxopiperidin-3-yl)benzofuran-2-yl)prop-2-yn-1-yl)picolinamide (20 mg, 33% yield) as a yellow solid. LC-MS (ESI): mass calcd. for C₄₄H₄₀N₈O₆, 776.2; m/z found, 777 [M+H]⁺. ¹H NMR (400 MHz, DMSO-d₆) 6 10.88 (s, 1H), 9.47-9.38 (m, 2H), 8.70 (dd, J=8.2, 2.0 Hz, 1H), 8.16 (d, J=8.3 Hz, 1H), 7.80 (s, 1H), 7.74 (s, 1H), 7.46 (d, J=8.3 Hz, 1H), 7.36-7.32 (m, 1H), 7.28 (d, J=9.2 Hz, 2H), 7.12 (d, J=7.4 Hz, 1H), 4.43 (d, J=5.8 Hz, 2H), 4.21 (dd, J=12.1, 4.9 Hz, 1H), 4.01-3.94 (m, 2H), 3.76 (s, 3H), 3.72 (s, 2H), 3.47-3.42 (m, 2H), 3.14-3.08 (m, 2H), 3.06-2.99 (m, 1H), 2.74-2.66 (m, 1H), 2.57-2.53 (m, 1H), 2.37-2.31 (m, 1H), 2.30-2.16 (m, 2H), 2.11 (s, 3H), 2.06-2.00 (m, 1H), 1.89-1.80 (m, 4H).

### Example B53. 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)-N-(3-(3-(2,6-dioxopiperidin-3-yl)benzofuran-5-yl)prop-2-yn-1-yl)picolinamide

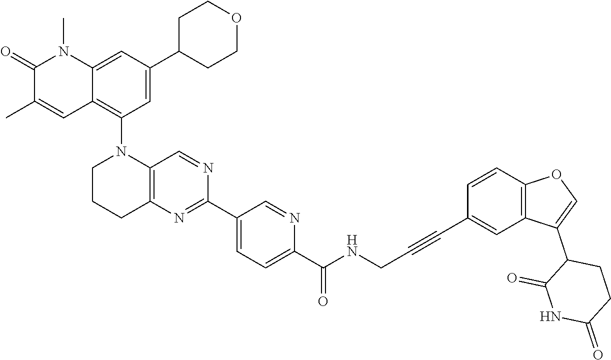 [SMILES: Cc1cc2c(N3CCCc4nc(-c5ccc(C(=O)NCC#Cc6ccc7occ(C8CCC(=O)NC8=O)c7c6)nc5)ncc43)cc(C3CCOCC3)cc2n(C)c1=O]

### Step A: 7-(3,6-dihydro-2H-pyran-4-yl)-5-methoxy-1,3-dimethylquinolin-2(1H)-one

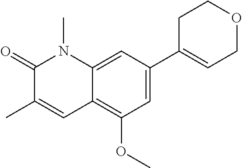 [SMILES: COc1cc(C2=CCOCC2)cc2c1cc(C)c(=O)n2C]

A mixture of 7-bromo-5-methoxy-1,3-dimethylquinolin-2(1H)-one (WO2022053967, 1.5 g, 5.3 mmol, 1.0 eq.), 2-(3,6-dihydro-2H-pyran-4-yl)-4,4,5,5-tetramethyl-1,3,2-dioxaborolane (3.4 g, 15.9 mmol, 3.0 eq.), Pd(dppf)Cl₂ (583 mg, 797 μmol, 0.15 eq.) and Na₂CO₃ (1.7 g, 15.9 mmol, 3.0 eq.) in 1,4-Dioxane (20 mL) and H₂O(2 mL) was stirred at 100° C. for 3 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-50% EtOAc and 10% DCM in PE) to give 7-(3,6-dihydro-2H-pyran-4-yl)-5-methoxy-1,3-dimethylquinolin-2(1H)-one (1.5 g, yield 95%) as a yellow solid. LC-MS (ESI): mass calcd. for C₁₇H₁₉NO₃, 285.14; m/z found, 286 [M+H]⁺.

### Step B: 5-methoxy-1,3-dimethyl-7-(tetrahydro-2H-pyran-4-yl)quinolin-2(1H)-one

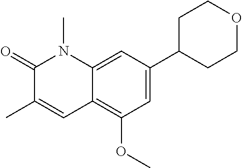 [SMILES: COc1cc(C2CCOCC2)cc2c1cc(C)c(=O)n2C]

To a solution of 7-(3,6-dihydro-2H-pyran-4-yl)-5-methoxy-1,3-dimethylquinolin-2(1H)-one (1.3 g, 4.6 mmol, 1.0 eq.) in EtOH (50 mL) and THF (25 mL) was added Pd/C (Palladium 10% on Carbon) at 25° C. The mixture was stirred at 50° C. for 5 hours under H₂. The resulting solution was filtered by celite pad and concentrated under reduced pressure.to give 5-methoxy-1,3-dimethyl-7-(tetrahydro-2H-pyran-4-yl)quinolin-2(1H)-one (1.2 g, yield 92%) as a yellow solid. LC-MS (ESI): mass calcd. for C₁₇H₂₁NO₃, 287.15; m/z found, 288 [M+H]+

### Step C: 5-hydroxy-1,3-dimethyl-7-(tetrahydro-2H-pyran-4-yl)quinolin-2(1H)-one

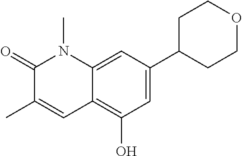 [SMILES: Cc1cc2c(O)cc(C3CCOCC3)cc2n(C)c1=O]

A mixture of 5-methoxy-1,3-dimethyl-7-(tetrahydro-2H-pyran-4-yl)quinolin-2(1H)-one (1.4 g, 4.9 mmol, 1.0 eq.) and EtSNa (410 mg, 4.9 mmol, 1.0 eq.) in DMF (30 mL) was stirred at 110° C. for 16 hour under N₂. The resulting solution was removed under reduced pressure. The residue was purified by flash column chromatography on silica gel (100-200 mesh silica gel, 0-80% EtOAc in PE) to give 5-hydroxy-1,3-dimethyl-7-(tetrahydro-2H-pyran-4-yl)quinolin-2(1H)-one (900 mg, yield 67%) as a yellow solid. LC-MS (ESI): mass calcd. for C₁₆H₁₉NO₃, 273.14; m/z found, 274 [M+H]⁺.

### Step D: 1,3-dimethyl-2-oxo-7-(tetrahydro-2H-pyran-4-yl)-1,2-dihydroquinolin-5-yl trifluoromethanesulfonate

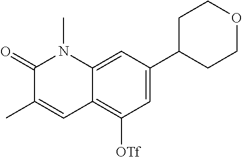 [SMILES: Cc1cc2c(OS(=O)(=O)C(F)(F)F)cc(C3CCOCC3)cc2n(C)c1=O]

To a solution of 5-hydroxy-1,3-dimethyl-7-(tetrahydro-2H-pyran-4-yl)quinolin-2(1H)-one (879 mg, 3.2 mmol, 1.0 eq.), Pyridine (1.0 mL, 12.8 mmol, 4.0 eq.) in DCM (15 mL) was added Tf₂O (1.8 g, 6.4 mmol, 2.0 eq.) at 0° C. The mixture was stirred at 0° C. for 2 hours under N₂. The resulting solution was diluted with 50 mL of water and extracted with EtOAc (50 mL×3). The combined organic layers were washed with brine (50 mL), dried over Na₂SO₄ and concentrated under reduced pressure. The residue was purified by flash column chromatography on silica gel (100-200 mesh silica gel, 0-100% EtOAc in PE) to give 1,3-dimethyl-2-oxo-7-(tetrahydro-2H-pyran-4-yl)-1,2-dihydroquinolin-5-yl trifluoromethanesulfonate (900 mg, yield 69%) as a white solid. LC-MS (ESI): mass calcd. for C₁₇H₁₈F₃NO₅S, 405.09; m/z found, 406 [M+H]⁺.

### Step E: ethyl (E)-3-(5-amino-2-chloropyrimidin-4-yl)acrylate

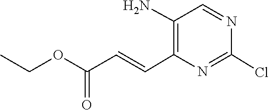

To a solution of 2,4-dichloropyrimidin-5-amine (50 g, 304 mmol, 1.0 eq.) in 1,4-Dioxane (400 mL) and H₂O(40 mL) was added ethyl (E)-3-(4,4,5,5-tetramethyl-1,3,2-dioxaborolan-2-yl)acrylate (84.5 g, 376 mmol, 1.2 eq.), K₂CO₃ (84 g, 608 mmol, 2.0 eq.) and X-Phos Pd G2 (10 g, 15.2 mmol, 0.05 eq.). The reaction mixture was stirred at 92° C. for 16 hours. The resulting solution was diluted with 300 ml of water and extracted with EtOAc (300 mL×3). The combined organic layers were washed with brine (500 mL×3), dried over Na₂SO₄ and concentrated under reduced pressure. The residue was purified by silica gel chromatography (100-200 mesh silica gel, 0-20% EtOAc in PE) to give ethyl (E)-3-(5-amino-2-chloropyrimidin-4-yl) acrylate (15 g, 65.8 mmol, 22%) as a yellow solid. LC-MS (ESI): mass calcd. for C₉H₁₀ClN₃O₂, 227.6; m/z found, 228.3 [M+H]⁺.

### Step F: 2-chloro-7,8-dihydropyrido[3,2-d]pyrimidin-6(5H)-one

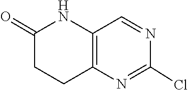 [SMILES: O=C1CCc2nc(Cl)ncc2N1]

To a solution of ethyl (E)-3-(5-amino-2-chloropyrimidin-4-yl) acrylate (15 g, 1, 65.9 mmol, 1.0 eq.) in EtOH (200 mL) was added Wilkinson's catalyst (12 g, 13.18 mmol, 0.2 eq.).

The reaction mixture was stirred at 60° C. for 10 hours. The resulting solution was diluted with 200 ml of water and extracted with EtOAc (200 mL×3). The combined organic layers were washed with brine (200 mL), dried over Na₂SO₄ and concentrated under reduced pressure. The residue was purified by silica gel chromatography (100-200 mesh silica gel, 0-50% EtOAc in PE) to give 2-chloro-7,8-dihydropyrido[3,2-d]pyrimidin-6(5H)-one (9 g, 49 mmol, 75%) as a yellow solid. LC-MS (ESI): mass calcd. for C₇H₆ClN₃₀, 183.6; m/z found, 184.5 [M+H]⁺.

### Step G: 2-chloro-5,6,7,8-tetrahydropyrido[3,2-d]pyrimidine

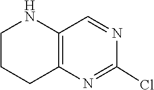 [SMILES: Clc1ncc2c(n1)CCCN2]

To a solution of 2-chloro-7,8-dihydropyrido[3,2-d]pyrimidin-6(5H)-one (9 g, 49.0 mmol, 1.0 eq.) in THF (200 mL) was added BH₃.THF (58.8 mL, 58.8 mmoL, 1.2 eq, 1.0 M in THF). The reaction mixture was stirred at 60° C. for 1 hour. The resulting solution was diluted with 20 ml of water and extracted with EtOAc (20 mL×3). The combined organic layers were washed with brine (60 mL), dried over Na₂SO₄ and concentrated under reduced pressure. The residue was purified by silica gel chromatography (100-200 mesh silica gel, 0-50% EtOAc in PE) to give the product 2-chloro-5,6,7,8-tetrahydropyrido[3,2-d]pyrimidine (3.5 g, 16.5 mmol, 43%) as a yellow solid. LC-MS (ESI): mass calcd. for C₇H₈ClN₃,169.6; m/z found, 170.4 [M+H]⁺.

### Step H: methyl 5-(5,6,7,8-tetrahydropyrido[3,2-d]pyrimidin-2-yl)picolinate

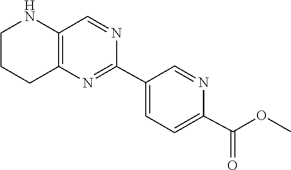 [SMILES: COC(=O)c1ccc(-c2ncc3c(n2)CCCN3)cn1]

A mixture of 2-chloro-5,6,7,8-tetrahydropyrido[3,2-d]pyrimidine (200 mg, 1.2 mmol, 1 eq.), Pd(dtbpf)Cl₂ (154 mg, 236 μmol 0.2 eq.), methyl 3-methyl-5-(4,4,5,5-tetramethyl-1,3,2-dioxaborolan-2-yl)picolinate (654 mg, 2.3 mmol, 2 eq.) and K₃PO₄ (751 mg, 3.5 mmol, 3 eq.) in 1,4-Dioxane (8 mL) and water (0.4 mL) was stirred at 100° C. for 2 hours under N₂. The resulting solution was diluted with 30 mL of water and extracted with EtOAc (30 mL×3). The combined organic layers were washed with brine (50 mL), dried over Na₂SO₄ and concentrated under reduced pressure. The residue was purified by flash column chromatography on silica gel (100-200 mesh silica gel, 0-85% EtOAc in PE) to give methyl 5-(5,6,7,8-tetrahydropyrido[3,2-d]pyrimidin-2-yl)picolinate (200 mg, yield 60%) as a yellow solid.. LC-MS (ESI): mass calcd. for C₁₄H₁₄N₄O₂, 270.11; m/z found, 271 [M+H]⁺.

### Step I: methyl 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)picolinate

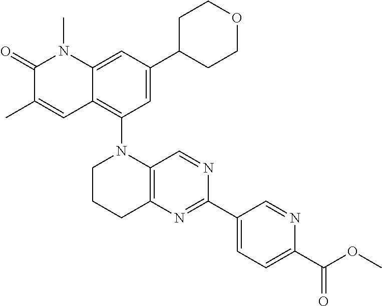

A mixture of methyl 5-(5,6,7,8-tetrahydropyrido[3,2-d]pyrimidin-2-yl)picolinate (180 mg, 666 μmol, 1 eq.), 1,3-dimethyl-2-oxo-7-(tetrahydro-2H-pyran-4-yl)-1,2-dihydroquinolin-5-yl trifluoromethanesulfonate (297 mg, 733 μmol, 1.1 eq.), X-Phos (64 mg, 133 μmol, 0.2 eq.), Pd₂(dba)₃ (122 mg 133 μmol, 0.2 eq.) and Cs₂CO₃ (649 mg, 2.0 mmol, 3 eq.) in Tol (5 mL). The mixture was stirred at 100° C. for 6 hours under N₂. The resulting solution was diluted with 20 mL of water and extracted with EtOAc (20 mL×3). The combined organic layers were washed with brine (100 mL), dried over Na₂SO₄ and concentrated under reduced pressure. The residue was purified by flash column chromatography on silica gel (100-200 mesh silica gel, 0-85% EtOAc in PE) to give methyl 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)picolinate (50 mg, yield 14%) as a yellow solid. LC-MS (ESI): mass calcd. for C₃₀H₃₁N₅O₄, 525.24; m/z found, 526 [M+H]⁺.

### Step J: lithium 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)picolinate

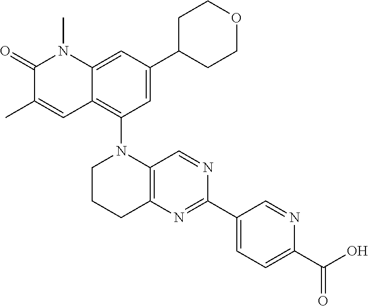 [SMILES: Cc1cc2c(N3CCCc4nc(-c5ccc(C(=O)O)nc5)ncc43)cc(C3CCOCC3)cc2n(C)c1=O]

To a solution of methyl 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)picolinate (50 mg, 95 μmol, 1.0 eq.) in THF (3 mL) and H₂O(3 mL) was added LiGH (4 mg, 190 μmol, 1.0 eq.) at room temperature. The mixture was stirred at room temperature for 1 hour. Then the mixture was concentrated under reduced pressure to give lithium 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)picolinate (50 mg, crude) as a yellow solid. LC-MS (ESI): mass calcd. for C₂₉H₂₉N₅O₄, 517.2; m/z found, 512.2 [M+H]⁺.

### Step K. 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)-N-(3-(3-(2,6-dioxopiperidin-3-yl)benzofuran-5-yl)prop-2-yn-1-yl)picolinamide

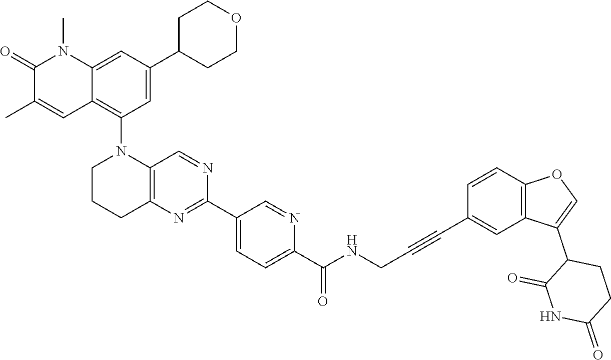 [SMILES: Cc1cc2c(N3CCCc4nc(-c5ccc(C(=O)NCC#Cc6ccc7occ(C8CCC(=O)NC8=O)c7c6)nc5)ncc43)cc(C3CCOCC3)cc2n(C)c1=O]

To a solution of 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)picolinic acid (150 mg, 1 Eq, 293 μmol),3-(5-(3-aminoprop-1-yn-1-yl)benzofuran-3-yl)piperidine-2,6-dione (149 mg, 1.8 Eq, 528 μmol) and HATU (279 mg, 2.5 Eq, 733 μmol) in DMF (10.00 mL) was added DIPEA (113 mg, 3 Eq, 880 μmol)at 0° C. The mixture was stirred at 0° C. for 30 min. LCMS showed the reaction was completed. The reaction was washed with H₂O, extracted with EA. The organic layer was dried over any. Na₂SO₄, concentrated in vacuum to give crude product which was purified by HPLC to give the desired product 5-(5-(1,3-dimethyl-2-oxo-7-(tetrahydro-2H-pyran-4-yl)-1,2-dihydroquinolin-5-yl)-5,6,7,8-tetrahydropyrido[3,2-d]pyrimidin-2-yl)-N-(3-(3-(2,6-dioxopiperidin-3-yl)benzofuran-5-yl)prop-2-yn-1-yl)picolinamide (88.0 mg, 113 μmol, 38.7%) as a yellow solid. ¹H NMR (400 MHz, DMSO) δ 10.88 (s, 1H), 9.37 (d, J=1.8 Hz, 1H), 9.27 (t, J=6.0 Hz, 1H), 8.66 (dd, J=8.2, 2.0 Hz, 1H), 8.13 (d, J=8.2 Hz, 1H), 7.95 (s, 1H), 7.70 (s, 2H), 7.57 (d, J=8.5 Hz, 1H), 7.42 (s, 1H), 7.40 (s, 1H), 7.36 (dd, J=8.5, 1.3 Hz, 1H), 7.28 (s, 1H), 4.35 (d, J=5.8 Hz, 2H), 4.15 (dd, J=12.3, 4.8 Hz, 1H), 3.97 (d, J=11.4 Hz, 2H), 3.76-3.68 (m, 4H), 3.62-3.56 (m, 1H), 3.45 (td, J=11.3, 5.5 Hz, 2H), 3.13 (dd, J=8.9, 5.2 Hz, 2H), 2.97 (dt, J=10.4, 5.8 Hz, 1H), 2.75-2.67 (m, 1H), 2.59-2.53 (m, 1H), 2.38-2.28 (m, 2H), 2.25-2.17 (m, 1H), 2.10 (s, 3H), 2.07-1.96 (m, 1H), 1.82-1.73 (m, 4H).

### Characterization of Compounds:

The compounds in Table 4 are synthesized via the route with amide formation described in Scheme 9; the precursors for amide coupling are synthesized via the routes listed in Scheme 1-8.

| No. | LC-MS: [M + H]⁺ | ¹H-NMR |
|---|---|---|
| B20 | mass calcd. for | ¹HNMR (400 MHZ, DMSO-d₆) δ 10.89 (s, 1H), 9.32-9.26 |
| | C₄₄H₄₁N₇O₇, 779.3; m/z | (m, 2H), 8.55-8.51 (m, 1H), 8.17 (s, 1H), 8.13 (s, 1H), |
| | found, 780.3 [M + H]⁺. | 7.95 (s, 1H), 7.48 (d, J = 8.3 Hz, 1H), 7.37-7.32 (m, 1H), |
| | | 7.30 (s, 1H), 7.13 (d, J = 7.4 Hz, 1H), 7.07 (s, 1H), 6.83 (s, |
| | | 1H), 4.42 (d, J = 5.8 Hz, 2H), 4.27-4.20 (m, 1H), 4.15 (s, |
| | | 3H), 3.97-3.91 (m, 2H), 3.68 (s, 3H), 3.45-3.39 (m, 2H), |
| | | 2.92-2.84 (m, 1H), 2.78-2.69 (m, 1H), 2.68 (s, 3H), 2.57-2.52 |
| | | (m, 1H), 2.35-2.32 (m, 1H), 2.13 (s, 3H), 2.08-2.02 |
| | | (m, 1H), 1.75-1.69 (m, 4H). |
| B21 | mass calcd. for | ¹H NMR (400 MHZ, DMSO-d₆) δ 10.88 (s, 1H), 9.08 (t, J = |
| | C₄₂H₃₄FN₅O₆, 723.2; m/z | 5.9 Hz, 1H), 8.56 (s, 1H), 7.96 (s, 1H), 7.85 (s, 1H), 7.82 |
| | found, 724.2 [M + H]⁺. | (s, 1H), 7.72-7.66 (m, 2H), 7.57 (t, J = 8.6 Hz, 2H), 7.39-7.34 |
| | | (m, 1H), 7.31 (d, J = 7.3 Hz, 1H), 7.16 (d, J = 7.6 |
| | | Hz, 1H), 5.87 (d, J = 12.8 Hz, 1H), 4.59-4.50 (m, 1H), |
| | | 4.45-4.37 (m, 1H), 4.31 (d, J = 5.8 Hz, 2H), 4.20-4.11 |
| | | (m, 1H), 3.86-3.78 (m, 1H), 3.71 (s, 3H), 3.68-3.63 (m, |
| | | 1H), 2.75-2.67 (m, 1H), 2.61 (s, 3H), 2.58-2.53 (m, 1H), |
| | | 2.37-2.30 (m, 1H), 2.15 (s, 3H), 2.11-2.05 (m, 1H). |
| B22 | mass calcd. for | ¹H NMR (400 MHZ, DMSO-d₆) δ 10.88 (s, 1H), 9.30 (d, J = |
| | C₄₃H₃₅F₂N₅O₅, 739.26; | 5.4 Hz, 1H), 8.57 (s, 1H), 8.13-8.07 (m, 1H), 7.98-7.90 |
| | m/z found, 740 [M + H]⁺. | (m, 2H), 7.71 (d, J = 4.5 Hz, 3H), 7.57 (d, J = 6.4 Hz, 2H), |
| | | 7.37 (s, 1H), 7.25 (d, J = 5.6 Hz, 1H), 7.18 (s, 1H), 6.78- |
| | | 6.50 (m, 1H), 6.21 (d, J = 4.6 Hz, 1H), 4.41-4.32 (m, 2H), |
| | | 4.16 (dd, 1H), 3.74-3.71 (m, 3H), 3.64-3.56 (m, 2H), |
| | | 3.04-2.98 (m, 2H), 2.76-2.68 (m, 1H), 2.61-2.55 (m, |
| | | 1H), 2.39-2.31 (m, 1H), 2.25-2.19 (m, 1H), 2.16-2.13 |
| | | (m, 3H), 2.12-2.06 (m, 2H). |
| B23 | mass calcd. for | ¹H NMR (400 MHZ, DMSO-d₆) δ 10.87 (s, 1H), 9.44-9.35 |
| | C₄₅H₃₉D₂N₇O₆, 777.32; | (m, 2H), 8.71-8.62 (m, 1H), 8.17-8.11 (m, 1H), 7.70 (s, |
| | m/z found, 778 [M + H]⁺. | 1H), 7.48-7.44 (m, 1H), 7.43-7.38 (m, 2H), 7.37-7.32 |
| | | (m, 1H), 7.30-7.26 (m, 2H), 7.12 (d, J = 7.3 Hz, 1H), 4.42 |
| | | (d, J = 6.0 Hz, 2H), 4.21 (dd, J = 12.2, 5.0 Hz, 1H), 4.06-3.85 |
| | | (m, 2H), 3.75-3.69 (m, 4H), 3.64-3.56 (m, 1H), 3.48-3.39 |
| | | (m, 2H), 3.18-3.09 (m, 2H), 2.70-2.65 (m, 1H), |
| | | 2.55-2.53 (m, 1H), 2.37-2.31 (m, 2H), 2.24-2.17 (m, |
| | | 1H), 2.10 (s, 3H), 2.05-2.00 (m, 1H), 1.81-1.72 (m, 3H). |
| B24 | mass calcd. for | ¹H NMR (400 MHZ, DMSO-d₆) δ 10.89 (s, 1H), 8.93-8.87 |
| | C₄₆H₄₃N₇O₇, 805.3; m/z | (m, 2H), 8.15-8.13 (m, 1H), 7.97 (s, 1H), 7.73-7.69 (m, |
| | found, 806.6 [M + H]⁺. | 2H), 7.59 (d, J = 8.5 Hz, 1H), 7.42-7.36 (m, 3H), 7.29- |
| | | 7.27 (m, 1H), 4.30 (d, J = 5.6 Hz, 2H), 4.19-4.13 (m, 1H), |
| | | 3.99-3.94 (m, 2H), 3.90 (s, 3H), 3.74-3.68 (m, 4H), 3.62-3.57 |
| | | (m, 1H), 3.48-3.45 (m, 2H), 3.15-3.09 (m, 2H), |
| | | 2.99-2.93 (m, 1H), 2.76-2.68 (m, 1H), 2.60-2.54 (m, |
| | | 1H), 2.36-2.29 (m, 2H), 2.24-2.18 (m, 1H), 2.12-2.07 |
| | | (m, 4H), 1.82-1.74 (m, 4H). |
| B25 | mass calcd. for | ¹H NMR (400 MHZ, DMSO-d₆) δ 10.96 (s, 1H), 9.45 (t, J = |
| | C₄₂H₄₀N₁₀O₅, 764.3; m/z | 5.9 Hz, 1H), 9.41 (d, J = 2.0 Hz, 1H), 8.70 (dd, J = 8.2, |
| | found, 765.3 [M + H]⁺. | 2.0 Hz, 1H), 8.46 (d, J = 5.8 Hz, 1H), 8.16 (d, J = 8.2 Hz, |
| | | 1H), 7.88 (s, 1H), 7.62 (d, J = 5.8 Hz, 1H), 7.55-7.54 (m, |
| | | 1H), 7.52 (s, 1H), 6.20 (s, 1H), 4.51-4.46 (m, 1H), 4.46- |
| | | 4.40 (m, 2H), 3.73-3.70 (m, 2H), 3.58-3.58 (m, 3H), 3.51-3.50 |
| | | (m, 2H), 3.11-3.08 (m, 2H), 2.65-2.56 (m, 2H), |
| | | 2.38-2.33 (m, 2H), 2.22-2.15 (m, 4H), 2.02 (s, 3H), 1.12 |
| | | (t, J = 6.9 Hz, 6H). |
| B26 | mass calcd. for | ¹H NMR (400 MHZ, DMSO-d₆) δ 10.89 (s, 1H), 9.20 (d, J = |
| | C₄₆H₄₃N₉O₆, 817.3; m/z | 1.5 Hz, 1H), 9.13 (t, J = 5.8 Hz, 1H), 8.42 (d, J = 1.3 Hz, |
| | found, 818.5 [M + H]⁺. | 1H), 7.96 (s, 1H), 7.87 (s, 1H), 7.72 (s, 1H), 7.60-7.54 (m, |
| | | 2H), 7.37 (dd, J = 8.6, 1.4 Hz, 1H), 6.38 (s, 1H), 4.46-4.40 |
| | | (m, 2H), 4.33 (d, J = 5.8 Hz, 2H), 4.16 (dd, J = 12.2, 4.8 |
| | | Hz, 1H), 3.96-3.89 (m, 2H), 3.77-3.70 (m, 2H), 3.58 (s, |
| | | 3H), 3.11-3.05 (m, 2H), 3.03-2.96 (m, 2H), 2.77-2.68 |
| | | (m, 1H), 2.64 (s, 3H), 2.60-2.54 (m, 1H), 2.38-2.29 (m, |
| | | 1H), 2.23-2.14 (m, 2H), 2.12-2.06 (m, 1H), 2.02 (s, 3H), |
| | | 1.88-1.78 (m, 2H), 1.76-1.68 (m, 2H). |
| B27 | mass calcd. for | ¹H NMR (400 MHZ, DMSO-d4) 8 10.88 (s, 1H), 9.42-9.37 |
| | C₄₄H₄₀FN₉O₅, 793.3; m/z | (m, 1H), 9.33-9.27 (m, 1H), 8.72-8.67 (m, 1H), 8.18-8.13 |
| | found, 794.3 [M + H]⁺. | (m, 1H), 7.96 (s, 1H), 7.89 (s, 1H), 7.71 (s, 1H), 7.59-7.55 |
| | | (m, 2H), 7.38-7.34 (m, 1H), 6.56 (s, 1H), 4.99-4.82 |
| | | (m, 1H), 4.39-4.33 (m, 2H), 4.18-4.13 (m, 1H), 3.83-3.77 |
| | | (m, 2H), 3.75-3.71 (m, 2H), 3.60 (s, 3H), 3.58-3.52 |
| | | (m, 2H), 3.13-3.07 (m, 2H), 2.73-2.67 (m, 1H), 2.61-2.55 |
| | | (m, 1H), 2.37-2.30 (m, 1H), 2.19 (s, 2H), 2.09 (s, |
| | | 1H), 2.03 (s, 3H), 1.98-1.88 (m, 2H), 1.71 (s, 2H). |
| B28 | mass calcd. for | ¹H NMR (400 MHZ, DMSO-d₆) δ 10.88 (s, 1H), 9.47-9.36 |
| | C₄₃H₃₉N₉O₆, 777.3; m/z | (m, 2H), 8.70 (dd, J = 8.2, 2.1 Hz, 1H), 8.16 (d, J = 8.2 Hz, |
| | found, 778.6 [M + H]⁺. | 1H), 7.87 (s, 1H), 7.58 (s, 1H), 7.46 (d, J = 8.4 Hz, 1H), |
| | | 7.37-7.31 (m, 1H), 7.29 (s, 1H), 7.12 (d, J = 7.3 Hz, 1H), |
| | | 6.51 (s, 1H), 4.43 (d, J = 5.9 Hz, 2H), 4.24-4.18 (m, 1H), |
| | | 3.76-3.67 (m, 6H), 3.60 (s, 3H), 3.56-3.50 (m, 4H), 3.13-3.06 |
| | | (m, 2H), 2.74-2.66 (m, 1H), 2.57-2.52 (m, 1H), |
| | | 2.39-2.30 (m, 1H), 2.19 (s, 2H), 2.06-2.00 (m, 4H). |
| B29 | mass calcd. for | ¹H NMR (400 MHZ, DMSO-d₆) δ 10.88 (s, 1H), 9.47-9.32 |
| | C₄₃H₃₉N₉O₆, 777; m/z | (m, 2H), 8.70 (dd, J = 8.3, 1.8 Hz, 1H), 8.16 (d, J = 8.3 Hz, |
| | found, 778 [M + H]⁺. | 1H), 7.87 (s, 1H), 7.58 (s, 1H), 7.46 (d, J = 8.3 Hz, 1H), |
| | | 7.33 (dd, J = 17.7, 9.6 Hz, 2H), 7.12 (d, J = 7.4 Hz, 1H), |
| | | 6.51 (s, 1H), 4.43 (d, J = 5.8 Hz, 2H), 4.21 (dd, J = 12.1, |
| | | 4.9 Hz, 1H), 3.71 (dd, J = 17.7, 4.8 Hz, 6H), 3.60 (s, 3H), |
| | | 3.54 (d, J = 4.6 Hz, 4H), 3.10 (t, J = 6.3 Hz, 2H), 2.74- |
| | | 2.66 (m, 1H), 2.57-2.54 (m, 1H), 2.38-2.31 (m, 1H), 2.19 |
| | | (m, 2H), 2.03 (m, 4H). |
| B30 | mass calcd. for | ¹H NMR (400 MHz, DMSO-d₆) δ 10.88 (s, 1H), 9.45-9.38 |
| | C₄₃H₃₉N₉O₆, 777; m/z | (m, 2H), 8.70 (dd, J = 8.2, 1.9 Hz, 1H), 8.16 (d, J = 8.3 Hz, |
| | found, 778 [M + H]⁺. | 1H), 7.87 (s, 1H), 7.58 (s, 1H), 7.46 (d, J = 8.3 Hz, 1H), |
| | | 7.34 (t, J = 7.9 Hz, 1H), 7.29 (s, 1H), 7.12 (d, J = 7.4 Hz, |
| | | 1H), 6.52 (s, 1H), 4.43 (d, J = 5.9 Hz, 2H), 4.21 (dd, J = |
| | | 12.1, 4.8 Hz, 1H), 3.76-3.67 (m, 6H), 3.60 (s, 3H), 3.56- |
| | | 3.51 (m, 4H), 3.10 (t, J = 6.3 Hz, 2H), 2.73-2.64 (m, 1H), |
| | | 2.58-2.52 (m, 1H), 2.38-2.30 (m, 1H), 2.24-2.15 (m, |
| | | 2H), 2.06-2.01 (m, 4H). |
| B31 | mass calcd. for | ¹H NMR (400 MHZ, DMSO-d₆) δ 10.88 (s, 1H), 9.32 (t, J = |
| | C₄₆H₄₃N₇O₆, 789.3; m/z | 5.8 Hz, 1H), 9.13-9.10 (m, 1H), 8.46 (dd, J = 8.3, 2.0 |
| | found, 790.5 [M + H]⁺. | Hz, 1H), 8.19 (d, J = 8.2 Hz, 1H), 7.96 (s, 1H), 7.76-7.74 |
| | | (m, 1H), 7.71-7.69 (m, 1H), 7.57 (d, J = 8.6 Hz, 1H), 7.42 |
| | | (s, 1H), 7.35 (dd, J = 8.6, 1.5 Hz, 1H), 7.32 (s, 1H), 7.28 (s, |
| | | 1H), 6.76 (s, 1H), 4.37 (d, J = 5.8 Hz, 2H), 4.15 (dd, J = |
| | | 12.2, 4.8 Hz, 1H), 3.98 (d, J = 9.8 Hz, 3H), 3.92-3.86 (m, |
| | | 1H), 3.72 (s, 3H), 3.70-3.62 (m, 2H), 3.49-3.43 (m, 2H), |
| | | 3.33 (s, 3H), 3.01-2.94 (m, 1H), 2.75-2.68 (m, 1H), 2.59-2.53 |
| | | (m, 1H), 2.37-2.30 (m, 1H), 2.12-2.07 (m, 4H), |
| | | 1.87-1.76 (m, 4H). |
| B32 | mass calcd. for | ¹H NMR (400 MHZ, DMSO-d₆) δ 10.95 (s, 1H), 9.45 (t, J = |
| | C₃₉H₃₃N₉O₅, 707.2; m/z | 5.9 Hz, 1H), 9.36 (d, J = 1.6 Hz, 1H), 8.71-8.64 (m, 1H), |
| | found, 708.2 [M + H]⁺. | 8.46 (d, J = 5.8 Hz, 1H), 8.17 (d, J = 8.3 Hz, 1H), 7.83 (s, |
| | | 1H), 7.71 (t, J = 8.2 Hz, 1H), 7.61 (d, J = 5.9 Hz, 1H), 7.55 |
| | | (d, J = 8.6 Hz, 1H), 7.51 (s, 1H), 7.33 (d, J = 7.6 Hz, 1H), |
| | | 6.81 (s, 1H), 4.49-4.43 (m, 3H), 4.06-4.02 (m, 2H), 3.71 |
| | | (s, 3H), 3.64-3.59 (m, 2H), 3.40 (s, 3H), 2.63-2.56 (m, |
| | | 2H), 2.39-2.33 (m, 1H), 2.20-2.14 (m, 1H), 2.11 (s, 3H). |
| B33 | mass calcd. for | ¹H NMR (400 MHZ, DMSO-d₆) δ 10.88 (s, 1H), 9.37-9.29 |
| | C₄₀H₃₄N₈O₅, 706.7; m/z | (m, 2H), 8.67 (d, J = 8.2 Hz, 1H), 8.17 (d, J = 8.2 Hz, 1H), |
| | found, 707.3 [M + H]⁺. | 7.96 (s, 1H), 7.83 (s, 1H), 7.74-7.69 (m, 2H), 7.56 (t, J = |
| | | 7.7 Hz, 2H), 7.35 (t, J = 8.1 Hz, 2H), 6.79 (s, 1H), 4.37 (d, |
| | | J = 5.7 Hz, 2H), 4.17-4.14 (m, 2H), 3.79-3.72 (m, 2H), |
| | | 3.71 (s, 3H), 3.62 (d, J = 10.9 Hz, 1H), 3.41 (s, 3H), 2.76- |
| | | 2.69 (m, 1H), 2.59-2.54 (m, 1H), 2.38-2.30 (m, 1H), 2.11 |
| | | (s, 3H), 2.10-2.03 (m, 1H). |
| B34 | mass calcd. for | ¹H NMR (400 MHZ, DMSO-d₆) δ 10.88 (s, 1H), 9.44 (s, |
| | C₄₀H₃₃N₇O₅, 691.8; m/z | 1H), 9.25 (s, 1H), 8.51 (d, J = 8.4 Hz, 1H), 8.13 (d, J = 8.4 |
| | found, 692.8 [M + H]⁺. | Hz, 1H), 8.02 (s, 1H), 7.66 (d, J = 3.9 Hz, 2H), 7.54 (dd, J = |
| | | 7.9, 3.1 Hz, 1H), 7.46 (d, J = 8.2 Hz, 1H), 7.34 (t, J = 7.9 |
| | | Hz, 1H), 7.27 (dd, J = 9.8, 4.9 Hz, 2H), 7.11 (d, J = 7.4 Hz, |
| | | 1H), 4.41 (d, J = 5.7 Hz, 2H), 4.21 (dd, J = 12.0 Hz, 1H), |
| | | 3.97-3.78 (m, 1H), 3.78-3.63 (m, 4H), 3.12-2.91 (m, |
| | | 2H), 2.77-2.65 (m, 1H), 2.38-2.32 (m, 2H), 2.28-2.20 |
| | | (m, 1H), 2.09 (s, 3H), 2.06-1.99 (m, 2H). |
| B35 | mass calcd. for | ¹H NMR (400 MHZ, DMSO-d₆) δ 10.96 (s, 1H), 9.50-9.36 |
| | C₄₁H₃₆N₁₀O₅, 748.3; m/z | (m, 2H), 8.70 (dd, J = 8.2, 2.1 Hz, 1H), 8.46 (d, J = 5.9 Hz, |
| | found, 748.8 [M + H]⁺. | 1H), 8.16 (d, J = 8.2 Hz, 1H), 7.84 (s, 1H), 7.62 (d, J = 5.8 |
| | | Hz, 1H), 7.54 (d, J = 17.6 Hz, 2H), 6.05 (s, 1H), 4.48-4.44 |
| | | (m, 3H), 4.00 (m, 4H), 3.73-3.68 (m, 2H), 3.56 (s, 3H), |
| | | 3.10-3.04 (m, 2H), 2.60-2.52 (m, 2H), 2.38-2.31 (m, |
| | | 3H), 2.18-2.13 (m, 3H), 2.02 (s, 3H). |
| B36 | mass calcd. for | ¹H NMR (400 MHZ, DMSO-d₆) δ 10.89 (s, 1H), 9.33 (t, J = |
| | C₄₀H₃₂ClN₇O₅, 725.22; | 5.7 Hz, 1H), 9.24 (d, J = 1.7 Hz, 1H), 8.54 (d, J = 1.7 Hz, |
| | m/z found, 726.22 | 1H), 7.75 (s, 1H), 7.71 (t, J = 8.1 Hz, 1H), 7.58 (d, J = 8.6 |
| | [M + H]⁺. | Hz, 1H), 7.48 (d, J = 8.3 Hz, 1H), 7.40 (s, 1H), 7.37-7.33 |
| | | (m, 1H), 7.31 (d, J = 6.6 Hz, 2H), 7.14 (s, 1H), 4.42-4.31 |
| | | (m, 2H), 4.25-4.20 (m, 1H), 3.72-3.65 (m, 4H), 3.62-3.55 |
| | | (m, 1H), 3.12-3.01 (m, 2H), 2.72-2.67 (m, 1H), 2.55-2.41 |
| | | (m, 1H), 2.38-2.32 (m, 2H), 2.21-2.13 (m, 1H), |
| | | 2.12 (s, 3H), 2.07-2.02 (m, 1H). |
| B37 | mass calcd. for | ¹H NMR (400 MHZ, DMSO-d₆) δ 10.88 (s, 1H), 9.43-9.35 |
| | C₄₅H₄₁N₇O₆, 775.3; m/z | (m, 2H), 8.69-8.63 (m, 1H), 8.14 (d, J = 8.2 Hz, 1H), 7.70 |
| | found, 776.3 [M + H]⁺. | (s, 1H), 7.48-7.44 (m, 1H), 7.43-7.39 (m, 2H), 7.36-7.32 |
| | | (m, 1H), 7.30-7.27 (m, 2H), 7.13-7.10 (m, 1H), 4.45-4.39 |
| | | (m, 2H), 4.23-4.18 (m, 1H), 3.99-3.94 (m, 2H), |
| | | 3.74-3.69 (m, 4H), 3.63-3.57 (m, 1H), 3.48-3.42 (m, |
| | | 2H), 3.16-3.10 (m, 2H), 2.99-2.93 (m, 1H), 2.73-2.66 |
| | | (m, 1H), 2.56-2.52 (m, 1H), 2.37-2.29 (m, 2H), 2.24-2.18 |
| | | (m, 1H), 2.10 (s, 3H), 2.05-2.00 (m, 1H), 1.82-1.75 |
| | | (m, 4H). |
| B38 | mass calcd. for | ¹H NMR (400 MHZ, DMSO-d₆) δ 10.88 (s, 1H), 9.51-9.25 |
| | C₄₅H₄₁N₇O₆, 775.3; m/z | (m, 2H), 8.66 (dt, J = 8.3, 2.1 Hz, 1H), 8.14 (d, J = 8.2 Hz, |
| | found, 776.3 [M + H]⁺. | 1H), 7.70 (s, 1H), 7.46 (d, J = 8.4 Hz, 1H), 7.43-7.38 (m, |
| | | 2H), 7.34 (t, J = 7.9 Hz, 1H), 7.28 (d, J = 2.8 Hz, 2H), 7.12 |
| | | (d, J = 7.4 Hz, 1H), 4.42 (d, J = 6.0 Hz, 2H), 4.21 (dd, J = |
| | | 12.2, 4.9 Hz, 1H), 4.04-3.84 (m, 2H), 3.72 (s, 3H), 3.63-3.53 |
| | | (m, 1H), 3.53-3.42 (m, 2H), 3.19-3.07 (m, 2H), 3.03-2.93 |
| | | (m, 1H), 2.82-2.65 (m, 1H), 2.59-2.53 (m, 2H), |
| | | 2.43-2.28 (m, 2H), 2.26-2.15 (m, 1H), 2.10 (s, 3H), 2.04-2.00 |
| | | (m, 1H), 1.96-1.63 (m, 4H). |
| B39 | mass calcd. for | ¹H NMR (400 MHz, DMSO-d₆) δ 10.88 (s, 1H), 9.44-9.34 |
| | C₄₅H₄₁N₇O₆, 775.3; m/z | (m, 2H), 8.70-8.62 (m, 1H), 8.14 (d, J = 8.3 Hz, 1H), 7.70 |
| | found, 776.5 [M + H]⁺. | (s, 1H), 7.48-7.39 (m, 3H), 7.36-7.31 (m, 1H), 7.30- |
| | | 7.25 (m, 2H), 7.12 (d, J = 7.4 Hz, 1H), 4.46-4.38 (m, 2H), |
| | | 4.24-4.18 (m, 1H), 4.00-3.92 (m, 2H), 3.75-3.68 (m, |
| | | 4H), 3.63-3.56 (m, 1H), 3.48-3.40 (m, 2H), 3.16-3.06 |
| | | (m, 2H), 3.01-2.92 (m, 1H), 2.74-2.66 (m, 1H), 2.58- |
| | | 2.53 (m, 1H), 2.38-2.29 (m, 2H), 2.25-2.17 (m, 1H), 2.10 |
| | | (s, 3H), 2.06-2.00 (m, 1H), 1.86-1.74 (m, 4H). |
| B40 | mass calcd. for | ¹H NMR (400 MHZ, DMSO-d₆) δ 10.88 (s, 1H), 9.48 (t, J = |
| | C₄₂H₃₇N₇O₅, 719.3; m/z | 5.9 Hz, 1H), 9.09 (d, J = 1.9 Hz, 1H), 8.45-8.41 (m, 1H), |
| | found, 720.2 [M + H]⁺. | 8.24 (d, J = 8.3 Hz, 1H), 7.76 (s, 1H), 7.48-7.42 (m, 2H), |
| | | 7.37-7.32 (m, 2H), 7.28 (s, 1H), 7.20 (s, 1H), 7.12 (d, J = |
| | | 7.3 Hz, 1H), 6.68 (s, 1H), 4.45 (d, J = 5.9 Hz, 2H), 4.21- |
| | | 4.17 (m, 1H), 4.07-4.00 (m, 1H), 3.86-3.81 (m, 1H), 3.75 |
| | | (d, J = 12.5 Hz, 1H), 3.70 (s, 3H), 3.66-3.60 (m, 1H), 3.38 |
| | | (s, 3H), 2.73-2.66-2.61 (m, 1H), 2.54-2.49 (m, 1H), |
| | | 2.49 (s, 3H), 2.34-2.29 (m, 1H), 2.10 (s, 3H), 2.06-2.00 |
| | | (m, 1H). |
| B41 | mass calcd. for | ¹H NMR (400 MHz, DMSO-d₆) δ 10.88 (s, 1H), 9.18 (t, J = |
| | C₄₆H₄₁N₅O₇, 775.86; m/z | 6.0 Hz, 1H), 8.87 (d, J = 1.8 Hz, 1H), 8.16 (dd, J = 8.2, |
| | found, 776.4 [M + H]⁺. | 2.3 Hz, 1H), 8.04 (d, J = 8.2 Hz, 1H), 7.95 (s, 1H), 7.77 (s, |
| | | 1H), 7.70 (s, 1H), 7.57 (d, J = 8.5 Hz, 1H), 7.36 (dd, J = |
| | | 5.6, 2.8 Hz, 3H), 7.21 (s, 1H), 7.11 (dd, J = 8.5, 2.1 Hz, |
| | | 1H), 6.15 (d, J = 8.5 Hz, 1H), 4.59-4.51 (m, 1H), 4.46-4.40 |
| | | (m, 1H), 4.34 (d, J = 5.8 Hz, 2H), 4.15 (dd, J = 12.2, |
| | | 4.7 Hz, 1H), 4.00-3.93 (m, 2H), 3.90-3.83 (m, 1H), 3.72 |
| | | (s, 3H), 3.68-3.60 (m, 1H), 3.49-3.41 (m, 2H), 3.00-2.90 |
| | | (m, 1H), 2.74-2.66 (m, 1H), 2.60-2.52 (m, 1H), 2.36-2.30 |
| | | (m, 1H), 2.12-2.05 (m, 4H), 1.81-1.72 (m, 4H). |
| B42 | mass calcd. for | ¹H NMR (400 MHZ, DMSO-d₆) δ 10.89 (s, 1H), 9.28-9.05 |
| | C₄₆H₄₃N₇O₆, 789.3; m/z | (m, 2H), 8.40 (s, 1H), 7.96 (s, 1H), 7.71 (s, 2H), 7.60-7.55 |
| | found, 790.3 [M + H]⁺. | (m, 1H), 7.44-7.32 (m, 3H), 7.28 (s, 1H), 4.32 (d, J = 5.2 |
| | | Hz, 2H), 4.18-4.13 (m, 1H), 4.00-3.94 (m, 2H), 3.74-3.71 |
| | | (m, 3H), 3.63-3.55 (m, 2H), 3.49-3.41 (m, 4H), 3.16-3.04 |
| | | (m, 3H), 2.99-2.94 (m, 1H), 2.73-2.68 (m, 1H), |
| | | 2.63 (s, 3H), 2.60-2.57 (m, 1H), 2.38-2.30 (m, 2H), 2.23-2.18 |
| | | (m, 1H), 2.04-1.95 (m, 1H), 1.82-1.67 (m, 4H). |
| B43 | mass calcd. for | ¹H NMR (400 MHZ, DMSO-d₆) δ 10.88 (s, 1H), 9.45-9.34 |
| | C₄₂H₃₇N₇O₅, 719.3; m/z | (m, 1H), 9.32-9.25 (m, 1H), 8.69-8.61 (m, 1H), 8.19-8.10 |
| | found, 720.3 [M + H]⁺. | (m, 1H), 7.96 (d, J = 4.8 Hz, 1H), 7.73-7.68 (m, 2H), |
| | | 7.60-7.55 (m, 1H), 7.42-7.34 (m, 3H), 7.21 (d, J = 3.7 |
| | | Hz, 1H), 4.40-4.33 (m, 2H), 4.20-4.13 (m, 1H), 3.71 (d, |
| | | J = 4.8 Hz, 3H), 3.37 (s, 3H), 3.13 (s, 2H), 2.83-2.76 (m, |
| | | 2H), 2.74-2.68 (m, 1H), 2.61-2.55 (m, 1H), 2.35-2.31 |
| | | (m, 1H), 2.27-2.19 (m, 1H), 2.15-2.08 (m, 4H), 1.28- |
| | | 1.24 (m, 3H). |
| B44 | mass calcd. for | ¹H NMR (400 MHZ, DMSO-d₆) δ 10.88 (s, 1H), 8.51 (s, |
| | C₄₇H₄₂N₆O₆, 786.3; m/z | 1H), 8.48-8.44 (m, 1H), 7.96 (s, 1H), 7.78-7.76 (m, 1H), |
| | found, 787.4 [M + H]⁺. | 7.72 (s, 1H), 7.69 (d, J = 8.1 Hz, 1H), 7.59 (d, J = 8.5 Hz, |
| | | 1H), 7.45-7.40 (m, 2H), 7.38 (s, 1H), 7.27 (s, 1H), 4.67 |
| | | (d, J = 2.7 Hz, 4H), 4.14 (dd, J = 12.2, 4.8 Hz, 1H), 3.99- |
| | | 3.94 (m, 2H), 3.72 (s, 3H), 3.70-3.66 (m, 2H), 3.44-3.41 |
| | | (m, 2H), 3.15-3.09 (m, 2H), 2.99-2.93 (m, 1H), 2.75- |
| | | 2.68 (m, 1H), 2.59-2.56 (m, 1H), 2.55-2.52 (m, 1H), 2.38- |
| | | 2.31 (m, 2H), 2.24-2.19 (m, 1H), 2.10 (s, 3H), 1.82- |
| | | 1.75 (m, 4H). |
| B45 | mass calcd. for | ¹H NMR (400 MHz, DMSO-d₆) δ 10.88 (s, 1H), 9.14-9.09 |
| | C₄₁H₃₅N₇O₅, 705.3; m/z | (m, 1H), 8.41-8.37 (m, 2H), 7.96 (s, 1H), 7.76 (s, 1H), |
| | found, 706.3 [M + H]⁺. | 7.72-7.68 (m, 2H), 7.59-7.55 (m, 2H), 7.41 (s, 1H), 7.38- |
| | | 7.35 (m, 1H), 7.32-7.29 (m, 1H), 4.34-4.28 (m, 2H), |
| | | 4.18-4.13 (m, 1H), 3.71 (s, 3H), 3.64-3.54 (m, 2H), 3.16- |
| | | 3.08 (m, 2H), 2.73-2.66 (m, 1H), 2.63 (s, 3H), 2.61- |
| | | 2.56 (m, 1H), 2.55-2.52 (m, 1H), 2.37-2.30 (m, 2H), 2.25- |
| | | 2.18 (m, 1H), 2.12 (s, 3H). |
| B46 | mass calcd. for | ¹H NMR (400 MHZ, DMSO-d₆) δ 10.88 (s, 1H), 9.40 (d, J = |
| | C₄₄H₄₂N₁₀O₇S, 854.3; | 1.5 Hz, 1H), 9.30 (t, J = 5.9 Hz, 1H), 8.70 (dd, J = 8.2, |
| | m/z found, 855.9 | 2.0 Hz, 1H), 8.16 (d, J = 8.2 Hz, 1H), 7.96 (s, 1H), 7.88 (s, |
| | [M + H]⁺. | 1H), 7.71 (s, 1H), 7.57 (d, J = 8.4 Hz, 2H), 7.36 (dd, J = |
| | | 8.5, 1.4 Hz, 1H), 6.59 (s, 1H), 4.36 (d, J = 5.9 Hz, 2H), 4.16- |
| | | 4.15 (m, 1H), 3.76-3.70 (m, 6H), 3.61 (s, 3H), 3.22- |
| | | 3.17 (m, 4H), 3.13-3.08 (m, 2H), 2.90 (s, 3H), 2.76-2.68 |
| | | (m, 1H), 2.59-2.53 (m, 1H), 2.37-2.30 (m, 1H), 2.24- |
| | | 2.16 (m, 2H), 2.09 (dd, J = 8.9, 4.0 Hz, 1H), 2.03 (s, 3H). |
| B47 | mass calcd. for | ¹H NMR (400 MHZ, DMSO-d₆) δ 10.87 (s, 1H), 9.46-9.37 |
| | C₄₄H₄₀N₈O₆, 776.3; m/z | (m, 2H), 8.71 (dd, J = 8.2, 2.1 Hz, 1H), 8.17 (d, J = 8.1 Hz, |
| | found, 777.8 [M + H]⁺. | 1H), 7.80 (s, 1H), 7.74 (d, J = 1.1 Hz, 1H), 7.46 (d, J = 8.4 |
| | | Hz, 1H), 7.37-7.31 (m, 1H), 7.28 (d, J = 8.1 Hz, 2H), 7.12 |
| | | (d, J = 7.4 Hz, 1H), 4.43 (d, J = 5.9 Hz, 2H), 4.25-4.16 |
| | | (m, 1H), 4.00-3.93 (m, 2H), 3.77 (s, 3H), 3.75-3.69 (m, |
| | | 2H), 3.50-3.41 (m, 2H), 3.15-3.09 (m, 2H), 3.07-3.00 |
| | | (m, 1H), 2.74-2.66 (m, 1H), 2.56-2.52 (m, 1H), 2.36- |
| | | 2.31 (m, 1H), 2.30-2.17 (m, 2H), 2.12 (s, 3H), 2.05-2.00 |
| | | (m, 1H), 1.89-1.82 (m, 4H). |
| B48 | mass calcd. for | ¹H NMR (400 MHZ, DMSO-d₆) δ 10.88 (s, 1H), 9.45-9.39 |
| | C₄₄H₄₀N₈O₆, 776.3; m/z | (m, 2H), 8.73-8.69 (m, 1H), 8.18-8.14 (m, 1H), 7.80 (s, |
| | found, 777.3 [M + H]⁺. | 1H), 7.74 (s, 1H), 7.48-7.45 (m, 1H), 7.36-7.32 (m, 1H), |
| | | 7.29-7.26 (m, 2H), 7.13-7.10 (m, 1H), 4.45-4.39 (m, |
| | | 2H), 4.21 (dd, J = 12.1, 4.8 Hz, 1H), 3.99-3.94 (m, 2H), |
| | | 3.77 (s, 3H), 3.72 (s, 2H), 3.48-3.44 (m, 2H), 3.14-3.10 |
| | | (m, 2H), 3.06-3.01 (m, 1H), 2.75-2.68 (m, 1H), 2.56- |
| | | 2.53 (m, 1H), 2.36-2.32 (m, 1H), 2.19 (s, 1H), 2.12 (s, |
| | | 3H), 2.08-1.94 (m, 2H), 1.88-1.82 (m, 4H). |
| B49 | mass calcd. for | ¹H NMR (400 MHZ, DMSO-d₆) δ 10.88 (s, 1H), 9.46-9.39 |
| | C₄₄H₄₀N₈O₆, 776.3; m/z | (m, 2H), 8.74-8.67 (m, 1H), 8.16 (d, J = 8.2 Hz, 1H), 7.80 |
| | found, 777.5 [M + H]⁺. | (s, 1H), 7.75-7.72 (m, 1H), 7.46 (d, J = 8.3 Hz, 1H), 7.36- |
| | | 7.32 (m, 1H), 7.29 (s, 1H), 7.27 (s, 1H), 7.12 (d, J = 7.3 |
| | | Hz, 1H), 4.43 (d, J = 5.9 Hz, 2H), 4.21 (dd, J = 12.2, 4.8 |
| | | Hz, 1H), 3.99-3.94 (m, 2H), 3.77 (s, 3H), 3.75-3.70 (m, |
| | | 2H), 3.47 (dd, J = 6.9, 4.4 Hz, 2H), 3.14-3.09 (m, 2H), |
| | | 3.07-3.01 (m, 1H), 2.73-2.67 (m, 1H), 2.55-2.53 (m, |
| | | 1H), 2.36-2.31 (m, 1H), 2.26-2.19 (m, 1H), 2.12 (s, 3H), |
| | | 2.06-1.96 (m, 2H), 1.89-1.82 (m, 4H). |
| B50 | mass calcd. for | ¹H NMR (400 MHZ, DMSO-d₆) δ 10.89 (s, 1H), 8.90 (dd, |
| | C₄₁H₃₅N₇O₆, 721.8; m/z | J = 11.4, 3.6 Hz, 2H), 8.14 (d, J = 1.4 Hz, 1H), 7.97 (s, |
| | found, 722.6 [M+H]″. | 1H), 7.75 (s, 1H), 7.72 (d, J = 1.5 Hz, 1H), 7.69 (d, J = 8.0 |
| | | Hz, 1H), 7.58 (t, J = 7.6 Hz, 2H), 7.41 (s, 1H), 7.37 (dd, J = |
| | | 8.5, 1.6 Hz, 1H), 7.30 (d, J = 7.4 Hz, 1H), 4.30 (d, J = |
| | | 5.6 Hz, 2H), 4.16 (dd, J = 12.2, 4.9 Hz, 1H), 3.89 (s, 3H), |
| | | 3.71 (s, 3H), 3.71-3.67 (m, 1H), 3.64-3.58 (m, 1H), |
| | | 3.18-3.10 (m, 2H), 2.75-2.68 (m, 1H), 2.61-2.54 (m, |
| | | 1H), 2.38-2.30 (m, 2H), 2.24-2.17 (m, 1H), 2.12 (s, |
| | | 3H), 2.11-2.05 (m, 1H). |
| B51 | mass calcd. for | ¹H NMR (400 MHZ, DMSO-d₆) δ 10.88 (s, 1H), 9.45 (d, J = |
| | C₄₂H₃₈N₁₀O₆, 778.8; m/z | 2.0 Hz, 1H), 9.33 (t, J = 5.9 Hz, 1H), 8.75 (dd, J = 8.2, |
| | found, 779.8 [M + H]⁺. | 2.1 Hz, 1H), 8.43 (s, 1H), 8.18 (d, J = 8.2 Hz, 1H), 7.96 (s, |
| | | 1H), 7.71 (d, J = 1.6 Hz, 1H), 7.57 (d, J = 8.5 Hz, 1H), 7.51 |
| | | (d, J = 1.4 Hz, 1H), 7.36 (dd, J = 8.6, 1.7 Hz, 1H), 4.37 (d, |
| | | J = 6.0 Hz, 2H), 4.15 (dd, J = 12.2, 4.9 Hz, 1H), 3.99-3.84 |
| | | (m, 2H), 3.79-3.70 (m, 4H), 3.69-3.63 (m, 4H), 3.59 (s, |
| | | 3H), 3.12-3.02 (m, 2H), 2.74-2.65 (m, 1H), 2.61-2.54 |
| | | (m, 1H), 2.39-2.27 (m, 2H), 2.18-2.02 (m, 2H), 2.00 (s, |
| | | 3H). |
| B52 | mass calcd. for | ¹H NMR (400 MHZ, DMSO-d₆) δ 10.88 (s, 1H), 9.40- |
| | C₄₈H₄₆N₈O₆, 830.9; m/z | 9.34 (m, 1H), 9.28 (t, J = 5.9 Hz, 1H), 8.66 (dd, J = 8.2, |
| | found, 831.8 [M + H]⁺. | 2.1 Hz, 1H), 8.14 (d, J = 8.2 Hz, 1H), 7.96 (s, 1H), 7.71 (s, |
| | | 2H), 7.57 (d, J = 8.5 Hz, 1H), 7.43-7.39 (m, 2H), 7.38- |
| | | 7.34 (m, 1H), 7.28 (s, 1H), 4.59 (d, J = 12.1 Hz, 1H), 4.36 |
| | | (d, J = 5.9 Hz, 2H), 4.15 (dd, J = 12.2, 4.9 Hz, 1H), 3.99 |
| | | (d, J = 12.4 Hz, 1H), 3.72 (s, 3H), 3.62-3.57 (m, 1H), |
| | | 3.16-3.09 (m, 3H), 2.99-2.93 (m, 1H), 2.74-2.68 (m, |
| | | 1H), 2.64-2.55 (m, 2H), 2.38-2.30 (m, 4H), 2.25-2.18 |
| | | (m, 1H), 2.10 (s, 3H), 1.90-1.82 |
| | | (m, 2H), 1.73-1.54 (m, 2H), 1.24 (s, 2H), 1.00 (dd, J = |
| | | 7.3, 5.3 Hz, 3H). |
| B53 | mass calcd. for | ¹H NMR (400 MHZ, DMSO-d₆) δ 10.87 (s, 1H), 9.37 (s, |
| | C₄₅H₄₁N₇O₆, 775.3; m/z | 1H), 9.27 (s, 1H), 8.66 (d, J = 8.2 Hz, 1H), 8.13 (d, J = 8.2 |
| | found, 776.3 [M + H]⁺. | Hz, 1H), 7.95 (s, 1H), 7.70 (s, 2H), 7.57 (d, J = 8.6 Hz, 1H), |
| | | 7.41 (d, J = 7.9 Hz, 2H), 7.36 (d, J = 8.1 Hz, 1H), 7.28 (s, |
| | | 1H), 4.35 (d, J = 5.7 Hz, 2H), 4.15 (dd, J = 12.1, 5.0 Hz, |
| | | 1H), 3.99-3.94 (m, 2H), 3.75-3.69 (m, 4H), 3.63-3.57 |
| | | (m, 1H), 3.49-3.41 (m, 2H), 3.16-3.09 (m, 2H), 3.00- |
| | | 2.92 (m, 1H), 2.74-2.67 (m, 1H), 2.59-2.53 (m, 1H), 2.35- |
| | | 2.29 (m, 2H), 2.26-2.19 (m, 1H), 2.10 (s, 3H), 2.08- |
| | | 2.02 (m, 1H), 1.81-1.73 (m, 4H). |
| B54 | mass calcd. for | ¹H NMR (400 MHZ, DMSO-d₆) δ 10.88 (s, 1H), 9.37 (d, J = |
| | C₄₅H₄₁N₇O₆, 775.3; m/z | 1.5 Hz, 1H), 9.27 (t, J = 5.9 Hz, 1H), 8.66 (dd, J = 8.2, |
| | found, 776.3 [M + H]⁺. | 2.1 Hz, 1H), 8.13 (d, J = 8.3 Hz, 1H), 7.95 (s, 1H), 7.70 (s, |
| | | 2H), 7.57 (d, J = 8.6 Hz, 1H), 7.41 (d, J = 6.8 Hz, 2H), 7.36 |
| | | (dd, J = 8.6, 1.5 Hz, 1H), 7.28 (s, 1H), 4.35 (d, J = 5.9 Hz, |
| | | 2H), 4.15 (dd, J = 12.2, 4.8 Hz, 1H), 4.00-3.93 (m, 2H), |
| | | 3.76-3.68 (m, 4H), 3.63-3.56 (m, 1H), 3.49-3.41 (m, |
| | | 2H), 3.16-3.06 (m, 2H), 3.03-2.91 (m, 1H), 2.75-2.66 |
| | | (m, 1H), 2.61-2.53 (m, 1H), 2.39-2.28 (m, 2H), 2.25- |
| | | 2.17 (m, 1H), 2.13-2.05 (m, 4H), 1.83-1.75 (m, 4H). |
| B55 | mass calcd. for | ¹H NMR (400 MHZ, DMSO-d₆) δ 10.88 (s, 1H), 9.37 (s, |
| | C₄₅H₄₁N₇O₆, 775.3; m/z | 1H), 9.28 (t, J = 5.8 Hz, 1H), 8.66 (d, J = 8.3 Hz, 1H), 8.14 |
| | found, 776.3 [M + H]⁺. | (d, J = 8.2 Hz, 1H), 7.96 (s, 1H), 7.71 (s, 2H), 7.57 (d, J = |
| | | 8.5 Hz, 1H), 7.41 (d, J = 6.2 Hz, 2H), 7.36 (d, J = 8.5 Hz, |
| | | 1H), 7.28 (s, 1H), 4.36 (d, J = 5.5 Hz, 2H), 4.19-4.13 (m, |
| | | 1H), 3.97 (d, J = 11.0 Hz, 2H), 3.77-3.69 (m, 4H), 3.62- |
| | | 3.57 (m, 1H), 3.48-3.44 (m, 2H), 3.17-3.09 (m, 2H), 2.99- |
| | | 2.92 (m, 1H), 2.74-2.67 (m, 1H), 2.60-2.55 (m, 1H), |
| | | 2.38-2.29 (m, 2H), 2.24-2.18 (m, 1H), 2.13-2.05 (m, |
| | | 4H), 1.83-1.74 (m, 4H). |
| B56 | mass calcd. for | ¹H NMR (400 MHZ, DMSO-d₆) δ 10.88 (s, 1H), 9.42 (d, J = |
| | C₄₂H₃₈N₈O₅, 734.30; m/z | 10.9 Hz, 2H), 8.70 (d, J = 8.0 Hz, 1H), 8.16 (d, J = 8.1 |
| | found, 735.30 [M + H]⁺. | Hz, 1H), 7.74 (d, J = 15.2 Hz, 2H), 7.46 (d, J = 8.1 Hz, 1H), |
| | | 7.34 (t, J = 7.8 Hz, 1H), 7.26 (d, J = 17.3 Hz, 2H), 7.12 (d, |
| | | J = 7.4 Hz, 1H), 4.43-4.33 (m, 2H), 4.21-4.11 (m, 1H), |
| | | 3.75-3.66 (m, 6H), 3.43-3.32(m, 3H), 3.12-3.01 (m, |
| | | 3H), 2.82-2.62 (m, 1H), 2.55-2.41 (m, 1H), 2.11 (s, 3H), |
| | | 1.30 (d, J = 7.6 Hz, 6H). |
| B57 | mass calcd. for | ¹H NMR (400 MHZ, DMSO-d₆) δ 10.77 (s, 1H), 9.35 (d, J = |
| | C₄₇H₄₈N₈O₅, 804.4; m/z | 1.4 Hz, 1H), 9.23 (t, J = 5.9 Hz, 1H), 8.65 (dd, J = 8.2, |
| | found, 804.8 [M + H]⁺. | 2.1 Hz, 1H), 8.12 (d, J = 8.2 Hz, 1H), 7.70 (s, 1H), 7.40 (d, |
| | | J = 7.3 Hz, 2H), 7.28 (s, 1H), 7.08-6.96 (m, 2H), 6.86 (dd, |
| | | J = 7.7, 1.3 Hz, 1H), 4.49-4.45 (m, 1H), 4.31-4.27 (m, |
| | | 2H), 3.97-3.92 (m, 2H), 3.71-3.67 (m, 4H), 3.65-3.55 |
| | | (m, 1H), 3.44-3.40 (m, 2H), 3.11-3.05 (m, 3H), 3.00- |
| | | 2.92 (m, 1H), 2.83-2.53 (m, 5H), 2.26-2.21 (m, 3H), 2.09- |
| | | 2.03 (m, 3H), 1.96-1.89 (m, 1H), 1.75-1.71 (m, 6H), |
| | | 1.47-1.42 (m, 1H), 1.25-1.20 (m, 1H). |
| B58 | mass calcd. for | ¹H NMR (400 MHZ, DMSO-d₆) δ 10.88 (s, 1H), 9.23 (t, J = |
| | C₄₆H₃₉FN₆O₇, 806.3; m/z | 5.9 Hz, 1H), 8.75 (d, J = 1.3 Hz, 1H), 8.08 (s, 2H), 7.96 |
| | found, 807.5 [M + H]⁺. | (s, 1H), 7.70 (s, 1H), 7.57 (d, J = 8.6 Hz, 2H), 7.36 (dd, J = |
| | | 8.5, 1.5 Hz, 1H), 7.17 (d, J = 7.6 Hz, 1H), 6.45 (d, J = 1.8 |
| | | Hz, 1H), 6.29 (s, 1H), 5.87 (d, J = 12.9 Hz, 1H), 4.74 (s, |
| | | 4H), 4.51 (t, J = 8.0 Hz, 1H), 4.41 (d, J = 11.0 Hz, 1H), 4.35 |
| | | (d, J = 6.0 Hz, 2H), 4.17-4.11 (m, 5H), 3.85-3.78 (m, |
| | | 1H), 3.60 (d, J = 20.4 Hz, 4H), 2.74-2.67 (m, 1H), 2.60- |
| | | 2.53 (m, 1H), 2.35-2.30 (m, 1H), 2.10-2.03 (m, 4H). |
| B59 | mass calcd. for | ¹H NMR (400 MHZ, DMSO-d₆) δ 10.88 (s, 1H), 9.33-9.25 |
| | C₄₄H₄₁N₇O₆, 763.3; m/z | (m, 1H), 9.21 (s, 1H), 8.43 (s, 1H), 8.19 (s, 2H), 7.66 (s, |
| | found, 764.3 [M + H]⁺. | 1H), 7.47 (d, J = 8.2 Hz, 1H), 7.41 (s, 1H), 7.34 (t, J = 7.8 |
| | | Hz, 1H), 7.29 (s, 1H), 7.22 (s, 1H), 7.12 (d, J = 7.3 Hz, 1H), |
| | | 4.40 (d, J = 5.3 Hz, 2H), 4.25-4.19 (m, 1H), 3.96 (d, J = |
| | | 10.6 Hz, 2H), 3.72 (s, 3H), 3.48-3.45 (m, 2H), 3.42 (s, |
| | | 3H), 2.99-2.91 (m, 1H), 2.74-2.68 (m, 1H), 2.65 (s, 3H), |
| | | 2.57-2.54 (m, 1H), 2.38-2.29 (m, 1H), 2.08 (s, 3H), 2.06- |
| | | 1.99 (m, 1H), 1.82-1.72 (m, 4H). |
| B60 | mass calcd. for | ¹H NMR (400 MHZ, DMSO-d₆) δ 10.94 (s, 1H), 9.42 (t, J = |
| | C₄₅H₄₀FN₇O₆, 793.3; m/z | 5.6 Hz, 1H), 9.37 (s, 1H), 8.66 (d, J = 8.0 Hz, 1H), 8.14 |
| | found, 794.3 [M H]+. | (d, J = 8.2 Hz, 1H), 7.70 (s, 1H), 7.51 (d, J = 8.1 Hz, 1H), |
| | | 7.45 (d, J = 7.5 Hz, 1H), 7.40 (d, J = 5.9 Hz, 2H), 7.28 (s, |
| | | 1H), 7.24 (d, J = 7.3 Hz, 1H), 4.47 (d, J = 5.2 Hz, 2H), 4.26- |
| | | 4.19 (m, 1H), 3.97 (d, J = 10.9 Hz, 2H), 3.72 (s, 4H), 3.62- |
| | | 3.57 (m, 1H), 3.44 (s, 2H), 3.11 (s, 2H), 2.99-2.92 (m, |
| | | 1H), 2.83-2.75 (m, 1H), 2.32-2.29 (m, 1H), 2.39-2.29 |
| | | (m, 1H), 2.20-2.24 (m, 2H), 2.10-2.14(m, 1H), 2.10 (s, |
| | | 3H), 1.70-1.80 (m, 4H). |
| B61 | mass calcd. for | ¹H NMR (400 MHZ, DMSO-d₆) δ 10.88 (s, 1H), 9.39 (d, J = |
| | C₄₃H₃₉N₉O₆, 777.3; m/z | 1.4 Hz, 1H), 9.29 (t, J = 5.9 Hz, 1H), 8.68 (dd, J = 8.2, |
| | found, 778.2 [M + H]⁺. | 2.1 Hz, 1H), 8.15 (d, J = 8.3 Hz, 1H), 7.96 (s, 1H), 7.72 (d, |
| | | J = 11.9 Hz, 2H), 7.57 (d, J = 8.5 Hz, 1H), 7.52 (d, J = 0.9 |
| | | Hz, 1H), 7.36 (dd, J = 8.5, 1.5 Hz, 1H), 6.84 (s, 1H), 4.36 |
| | | (d, J = 5.9 Hz, 2H), 4.15 (dd, J = 12.2, 4.8 Hz, 1H), 3.75- |
| | | 3.69 (m, 5H), 3.66-3.57 (m, 8H), 3.10 (t, J = 6.3 Hz, 2H), |
| | | 2.76-2.67 (m, 1H), 2.59-2.53 (m, 1H), 2.38-2.17 (m, |
| | | 3H), 2.09-2.00 (m, 4H). |
| B62 | mass calcd. for | ¹H NMR (400 MHZ, DMSO-d₆) δ 10.88 (s, 1H), 9.45-9.38 |
| | C₄₃H₃₉N₉O₆, 777.3; m/z | (m, 1H), 9.30 (t, J = 6.0 Hz, 1H), 8.70 (dd, J = 8.2, 2.0 Hz, |
| | found, 778.3 [M + H]⁺. | 1H), 8.44 (s, 1H), 8.15 (d, J = 8.4 Hz, 1H), 7.96 (s, 1H), |
| | | 7.87 (s, 1H), 7.71 (s, 1H), 7.58-7.55 (m, 1H), 7.38-7.34 |
| | | (m, 1H), 6.52 (s, 1H), 4.36 (d, J = 5.9 Hz, 2H), 4.18-4.13 |
| | | (m, 1H), 3.77-3.72 (m, 2H), 3.71-3.68 (m, 4H), 3.60 (s, |
| | | 3H), 3.55-3.52 (m, 4H), 3.10 (t, J = 6.3 Hz, 2H), 2.73- |
| | | 2.67 (m, 1H), 2.59-2.53 (m, 1H), 2.35-2.31 (m, 1H), 2.23- |
| | | 2.17 (m, 2H), 2.11-2.06 (m, 1H), 2.03 (s, 3H). |
| B63 | mass calcd. for | ¹H NMR (400 MHZ, DMSO-d₆) δ 10.87 (s, 1H), 9.31-9.16 |
| | C₄₅H₃₉FN₆O₇, 794.3; m/z | (m, 1H), 8.75 (s, 1H), 8.08 (s, 2H), 7.95 (s, 1H), 7.70 (s, |
| | found, 795.3 [M + H]⁺. | 1H), 7.59-7.55 (m, 2H), 7.37-7.32 (m, 1H), 7.19-7.15 |
| | | (m, 1H), 6.45 (s, 1H), 6.28 (s, 1H), 5.91-5.85 (m, 1H), |
| | | 5.63-5.59 (m, 1H), 4.56-4.43 (m, 2H), 4.37-4.32 (m, |
| | | 3H), 4.17-4.13 (m, 2H), 3.90-3.86 (m, 2H), 3.81-3.77 |
| | | (m, 2H), 3.62 (s, 3H), 2.73-2.69 (m, 1H), 2.61-2.58 (m, |
| | | 1H), 2.35-2.31 (m, 1H), 2.06 (s, 3H), 1.99-1.94 (m, 1H), |
| | | 1.47-1.44 (m, 3H). |
| B64 | mass calcd. for | ¹H NMR (400 MHZ, DMSO-d₆) δ 10.87 (s, 1H), 9.36-9.30 |
| | C₄₅H₄₁N₇O₆, 775.3; m/z | (m, 1H), 9.25-9.21 (m, 1H), 8.54-8.49 (m, 1H), 8.22- |
| | found, 776.3 [M + H]⁺. | 8.12 (m, 2H), 7.96 (s, 1H), 7.71-7.69 (m, 1H), 7.65 (s, |
| | | 1H), 7.59-7.55 (m, 1H), 7.44 (s, 1H), 7.37-7.33 (m, 1H), |
| | | 7.29 (s, 1H), 4.35 (d, J = 5.9 Hz, 2H), 4.15 (dd, J = 12.2, |
| | | 4.8 Hz, 1H), 4.01-3.96 (m, 2H), 3.92-3.86 (m, 1H), 3.75- |
| | | 3.70 (m, 4H), 3.50-3.43 (m, 2H), 3.16-3.06 (m, 2H), |
| | | 3.01-2.93 (m, 1H), 2.74-2.67 (m, 1H), 2.59-2.54 (m, |
| | | 1H), 2.36-2.15 (m, 3H), 2.12-2.06 (m, 4H), 1.85-1.73 |
| | | (m, 4H). |
| B65 | mass calcd. for | ¹H NMR (400 MHZ, DMSO-d₆) δ 10.87 (s, 1H), 9.38 (s, |
| | C₄₀H₃₄N₈O₅, 706.3; m/z | 2H), 8.67 (dd, J = 8.2, 2.1 Hz, 1H), 8.11 (d, J = 8.2 Hz, 1H), |
| | found, 707.3 [M + H]⁺. | 7.81 (s, 1H), 7.69 (t, J = 8.2 Hz, 1H), 7.52 (d, J = 8.4 Hz, |
| | | 1H), 7.46 (d, J = 8.3 Hz, 1H), 7.31 (dd, J = 14.6, 6.8 Hz, |
| | | 3H), 7.12 (d, J = 7.3 Hz, 1H), 6.87 (s, 1H), 4.42 (d, J = 6.0 |
| | | Hz, 2H), 4.23-4.19 (m, 1H), 4.00-3.91 (m, 2H), 3.71 (s, |
| | | 3H), 3.66-3.61 (m, 2H), 3.37 (s, 3H), 2.73-2.67 (m, 2H), |
| | | 2.34-2.31 (m, 1H), 2.11 (s, 3H), 2.06-2.00 (m, 1H). |
| B66 | mass calcd. for | ¹H NMR (400 MHZ, DMSO-d₆) δ 10.88 (s, 1H), 9.50-9.44 |
| | C₄₄H₃₅N₇O₆, 757.2; m/z | (m, 1H), 9.38-9.31 (m, 1H), 9.03 (s, 1H), 8.81 (s, 1H), |
| | found, 758.3 [M + H]⁺. | 8.77 (dd, J = 8.2, 2.2 Hz, 1H), 8.26-8.19 (m, 2H), 8.00- |
| | | 7.95 (m, 2H), 7.74-7.71 (m, 1H), 7.70-7.67 (m, 1H), 7.60- |
| | | 7.56 (m, 1H), 7.40-7.35 (m, 1H), 7.10-7.06 (m, 1H), |
| | | 6.30 (s, 1H), 4.65-4.58 (m, 1H), 4.38 (d, J = 5.9 Hz, 2H), |
| | | 4.32-4.26 (m, 2H), 4.15 (dd, J = 7.4 Hz, 1H), 3.83-3.82 |
| | | (m, 2H), 3.63 (s, 3H), 2.76-2.67 (m, 1H), 2.62-2.52 (m, |
| | | 1H), 2.37-2.30 (m, 1H), 2.11-2.05 (m, 1H), 1.90-1.86 |
| | | (m, 3H). |
| B67 | mass calcd. for | ¹H NMR (400 MHZ, DMSO-d₆) δ 10.88 (s, 1H), 9.41 (t, J = |
| | C₄₀H₃₃N₇O₅, 691.2; m/z | 6.0 Hz, 1H), 9.37 (d, J = 1.5 Hz, 1H), 8.66 (dd, J = 8.3, |
| | found, 692.2 [M + H]⁺. | 2.0 Hz, 1H), 8.14 (d, J = 8.2 Hz, 1H), 7.75 (s, 1H), 7.70 (t, |
| | | J = 8.1 Hz, 1H), 7.57 (d, J = 8.6 Hz, 1H), 7.46 (d, J = 8.3 |
| | | Hz, 1H), 7.41 (s, 1H), 7.36-7.29 (m, 3H), 7.12 (d, J = 7.4 |
| | | Hz, 1H), 4.42 (d, J = 5.8 Hz, 2H), 4.21 (dd, J = 12.2, 4.9 |
| | | Hz, 1H), 3.74-3.68 (m, 4H), 3.63-3.58 (m, 1H), 3.17- |
| | | 3.10 (m, 2H), 2.74-2.66 (m, 1H), 2.57-2.53 (m, 1H), 2.39- |
| | | 2.31 (m, 2H), 2.25-2.18 (m, 1H), 2.12 (s, 3H), 2.06- |
| | | 2.01 (m, 1H). |
| B68 | mass calcd. for | ¹H NMR (400 MHZ, DMSO-d₆) δ 10.88 (s, 1H), 9.42-9.39 |
| | C₄₂H₃₆FN₉O₅, 765.3; m/z | (m, 1H), 9.32-9.28 (m, 1H), 8.72-8.68 (m, 1H), 8.15 (d, |
| | found, 766.5 [M + H]⁺. | J = 8.8 Hz, 1H), 7.96 (s, 1H), 7.86 (s, 1H), 7.72-7.70 (m, |
| | | 1H), 7.60-7.56 (m, 2H), 7.38-7.35 (m, 1H), 6.21 (s, 1H), |
| | | 5.51 (d, J = 57.2 Hz, 1H), 4.38-4.29 (m, 4H), 4.15 (dd, J = |
| | | 12.2, 5.0 Hz, 1H), 4.11-4.02 (m, 2H), 3.73-3.70 (m, |
| | | 2H), 3.58 (s, 3H), 3.12-3.08 (m, 2H), 2.72-2.67 (m, 1H), |
| | | 2.59-2.56 (m, 1H), 2.35-2.31 (m, 1H), 2.23-2.16 (m, |
| | | 2H), 2.11-2.06 (m, 1H), 2.04-2.02 (m, 3H). |
| B69 | mass calcd. for | ¹H NMR (400 MHZ, DMSO-d₆) δ 10.88 (s, 1H), 9.38 (d, J = |
| | C₃₉H₃₂N₈O₅, 692.2; m/z | 1.6 Hz, 1H), 9.28 (t, J = 5.9 Hz, 1H), 8.96 (s, 1H), 8.73- |
| | found, 693.2 [M + H]⁺. | 8.60 (m, 1H), 8.51 (s, 1H), 8.14 (d, J = 8.3 Hz, 1H), 7.95 (s, |
| | | 1H), 7.80 (s, 1H), 7.71 (s, 1H), 7.61-7.49 (m, 2H), 7.40- |
| | | 7.31 (m, 1H), 4.35 (d, J = 5.9 Hz, 2H), 4.17-4.12 (m, 1H), |
| | | 3.69-3.62 (m, 2H), 3.23 (s, 3H), 3.17-3.09 (m, 2H), 2.74- |
| | | 2.67 (m, 1H), 2.58-2.53 (m, 1H), 2.38-2.29 (m, 2H), |
| | | 2.27-2.20 (m, 1H), 2.17 (s, 3H), 2.11-2.04 (m, 1H). |
| B70 | mass calcd. for | ¹H NMR (400 MHZ, DMSO-d₆) δ 10.88 (s, 1H), 9.40 (d, J = |
| | C₄₄H₄₀N₈O₆, 776.3; m/z | 1.2 Hz, 1H), 9.30 (t, J = 5.8 Hz, 1H), 8.75-8.64 (m, 1H), |
| | found, 777.3 [M + H]⁺. | 8.15 (d, J = 8.2 Hz, 1H), 7.96 (s, 1H), 7.86 (s, 1H), 7.74 (d, |
| | | J = 21.0 Hz, 2H), 7.59-7.55 (m, 1H), 7.37 (s, 1H), 7.29 (s, |
| | | 1H), 4.38 (s, 2H), 4.15 (dd, J = 12.2, 4.8 Hz, 1H), 3.94 (d, |
| | | J = 10.9 Hz, 2H), 3.76 (s, 2H), 3.67 (s, 3H), 3.48-3.40 (m, |
| | | 3H), 3.15-3.09 (m, 2H), 3.02-2.95 (m, 1H), 2.76-2.67 |
| | | (m, 1H), 2.60-2.54 (m, 1H), 2.38-2.29 (m, 1H), 2.25- |
| | | 2.16 (m, 2H), 2.12 (s, 3H), 1.84-1.75 (m, 4H). |
| B71 | mass calcd. for | ¹H NMR (400 MHZ, DMSO-d₆) δ 10.88 (s, 1H), 9.21 (d, J = |
| | C₄₄H₄₁N₉O₆, 792.3; m/z | 1.5 Hz, 1H), 9.13-9.10 (m, 1H), 8.43-8.41 (m, 1H), |
| | found, 793.3 [M + H]⁺. | 7.96 (s, 1H), 7.87 (s, 1H), 7.72-7.69 (m, 1H), 7.60-7.56 |
| | | (m, 2H), 7.40-7.34 (m, 1H), 6.51 (s, 1H), 4.33 (d, J = 5.7 |
| | | Hz, 2H), 4.19-4.12 (m, 1H), 3.75-3.71 (m, 2H), 3.69 (d, |
| | | J = 4.9 Hz, 4H), 3.60 (s, 3H), 3.55-3.51 (m, 4H), 3.09 (t, |
| | | J = 6.6 Hz, 2H), 2.75-2.67 (m, 1H), 2.64 (s, 3H), 2.58- |
| | | 2.53 (m, 1H), 2.36-2.30 (m, 1H), 2.24-2.16 (m, 2H), 2.11- |
| | | 2.06 (m, 1H), 2.03 (s, 3H). |
| B72 | mass calcd. for | ¹H NMR (400 MHZ, DMSO-d₆) δ 10.88 (s, 1H), 9.40 (s, |
| | C₄₃H₃₉N₉O₆, 777.3; m/z | 1H), 9.34-9.26 (m, 1H), 8.70 (d, J = 8.2 Hz, 1H), 8.16 (d, |
| | found, 778.3 [M + H]⁺. | J = 8.2 Hz, 1H), 7.96 (s, 1H), 7.88 (s, 1H), 7.72 (s, 1H), |
| | | 7.60-7.56 (m, 2H), 7.37 (d, J = 8.5 Hz, 1H), 6.52 (s, 1H), |
| | | 4.36 (d, J = 5.6 Hz, 2H), 4.18-4.12 (m, 1H), 3.77-3.73 |
| | | (m, 2H), 3.71-3.67 (m, 4H), 3.60 (s, 3H), 3.57-3.52 (m, |
| | | 4H), 3.13-3.08 (m, 2H), 2.73-2.67 (m, 1H), 2.60-2.56 |
| | | (m, 1H), 2.37-2.30 (m, 1H), 2.24-2.17 (m, 2H), 2.12- |
| | | 2.07 (m, 1H), 2.03 (s, 3H). |
| B73 | mass calcd. for | ¹H NMR (400 MHZ, DMSO-d₆) δ 10.88 (s, 1H), 9.40 (d, J = |
| | C₄₃H₃₉N₉O₆, 777.3; m/z | 1.5 Hz, 1H), 9.30 (t, J = 6.1 Hz, 1H), 8.70 (dd, J = 8.2, |
| | found, 778.3 [M + H]⁺. | 2.1 Hz, 1H), 8.15 (d, J = 8.1 Hz, 1H), 7.96 (s, 1H), 7.87 (s, |
| | | 1H), 7.71 (s, 1H), 7.57 (d, J = 8.1 Hz, 2H), 7.36 (dd, J = |
| | | 8.6, 1.6 Hz, 1H), 6.52 (s, 1H), 4.36 (d, J = 5.9 Hz, 2H), 4.15 |
| | | (dd, J = 12.3, 4.8 Hz, 1H), 3.75-3.67 (m, 6H), 3.60 (s, 3H), |
| | | 3.56-3.51 (m, 4H), 3.35-3.34 (m, 1H), 3.11-3.09 (m, |
| | | 1H), 2.75-2.67 (m, 1H), 2.58-2.54 (m, 1H), 2.39-2.27 |
| | | (m, 1H), 2.26-2.11 (m, 2H), 2.11-2.05 (m, 1H), 2.03 (s, |
| | | 3H). |
| B74 | mass calcd. for | ¹H NMR (400 MHZ, DMSO-d₆) δ 10.88 (s, 1H), 9.49-9.43 |
| | C₄₅H₃₇N₇O₆, 771.3; m/z | (m, 2H), 9.05 (s, 1H), 8.82-8.75 (m, 2H), 8.22 (d, J = 8.3 |
| | found, 772.8 [M + H]⁺. | Hz, 2H), 7.99-7.94 (m, 1H), 7.69 (dd, J = 7.1, 0.9 Hz, 1H), |
| | | 7.47 (d, J = 8.4 Hz, 1H), 7.37-7.32 (m, 1H), 7.30 (d, J = |
| | | 0.7 Hz, 1H), 7.15-7.11 (m, 2H), 6.71 (s, 1H), 4.45 (d, J = |
| | | 5.9 Hz, 2H), 4.25-4.19 (m, 1H), 3.74-3.69 (m, 4H), 3.67 |
| | | (s, 3H), 3.64-3.55 (m, 4H), 2.74-2.67 (m, 1H), 2.57- |
| | | 2.53 (m, 1H), 2.38-2.31 (m, 1H), 2.07-2.00 (m, 1H), 1.92- |
| | | 1.88 (m, 3H). |
| B76 | mass calcd. for | ¹H NMR (400 MHZ, DMSO-d₆) δ 10.88 (s, 1H), 9.40 (s, |
| | C₄₄H₄₀N₈O₆, 776.3; m/z | 1H), 9.30 (t, J = 5.7 Hz, 1H), 8.70 (dd, J = 8.2, 2.0 Hz, 1H), |
| | found, 777.8 [M + H]⁺. | 8.16 (d, J = 8.2 Hz, 1H), 7.96 (s, 1H), 7.80 (s, 1H), 7.72 (d, |
| | | J = 13.8 Hz, 2H), 7.57 (d, J = 8.5 Hz, 1H), 7.36 (dd, J = 8.5, |
| | | 1.5 Hz, 1H), 7.27 (s, 1H), 4.36 (d, J = 5.9 Hz, 2H), 4.15 (dd, |
| | | J = 12.3, 4.8 Hz, 1H), 3.99-3.95 (m, 2H), 3.77 (s, 3H), |
| | | 3.74-3.71 (m, 2H), 3.48-3.44 (m, 2H), 3.14-3.09 (m, |
| | | 2H), 3.06-3.00 (m, 1H), 2.75-2.68 (m, 1H), 2.58-2.54 |
| | | (m, 1H), 2.39-2.27 (m, 2H), 2.22-2.16 (m, 1H), 2.13- |
| | | 2.07 (m, 4H), 1.89-1.82 (m, 4H). |
| B77 | mass calcd. for | ¹H NMR (400 MHZ, DMSO-d₆) δ 10.88 (s, 1H), 9.24 (t, J = |
| | C₄₀H₃₂N₆O₆, 692.2; m/z | 6.0 Hz, 1H), 9.21 (d, J = 1.8 Hz, 1H), 8.49 (dd, J = 8.3, |
| | found, 693.4 [M + H]⁺. | 2.2 Hz, 1H), 8.07 (d, J = 8.3 Hz, 1H), 7.95 (s, 1H), 7.90 (s, |
| | | 1H), 7.71 (s, 2H), 7.70-7.65 (m, 1H), 7.57 (d, J = 8.5 Hz, |
| | | 1H), 7.53 (d, J = 8.6 Hz, 1H), 7.44 (s, 1H), 7.36 (dd, J = |
| | | 8.5, 1.5 Hz, 1H), 7.30 (d, J = 7.6 Hz, 1H), 4.69-4.64 (m, |
| | | 1H), 4.53-4.48 (m, 1H), 4.35 (d, J = 5.9 Hz, 2H), 4.15 (dd, |
| | | J = 12.2, 4.8 Hz, 1H), 3.89-3.83 (m, 1H), 3.73-3.69 (m, |
| | | 4H), 2.75-2.67 (m, 1H), 2.58-2.53 (m, 1H), 2.37-2.29 |
| | | (m, 1H), 2.14 (s, 3H), 2.10-2.06 (m, 1H). |
| B78 | mass calcd. for | ¹H NMR (400 MHZ, DMSO-d₆) δ 10.87 (s, 1H), 9.37 (s, |
| | C₄₄H₄₀N₈O₆, 776.3; m/z | 1H), 9.27 (t, J = 6.0 Hz, 1H), 8.66 (dd, J = 8.1, 2.0 Hz, 1H), |
| | found, 777.3 [M + H]⁺. | 8.13 (d, J = 8.2 Hz, 1H), 7.96 (d, J = 2.1 Hz, 1H), 7.71 (s, |
| | | 1H), 7.63 (t, J = 1.4 Hz, 1H), 7.57 (d, J = 8.5 Hz, 1H), 7.38 |
| | | (d, J = 9.5 Hz, 3H), 4.35 (d, J = 5.9 Hz, 2H), 4.15 (dd, J = |
| | | 12.3, 4.9 Hz, 1H), 4.01-3.92 (m, 2H), 3.83-3.70 (m, 2H), |
| | | 3.67 (s, 3H), 3.46-3.39 (m, 3H), 3.17-3.03 (m, 2H), 3.01- |
| | | 2.90 (m, 1H), 2.81-2.63 (m, 2H), 2.63-2.55 (m, 2H), |
| | | 2.33 (s, 3H), 2.26-2.16 (m, 2H), 2.16-2.05 (m, 1H), 1.86- |
| | | 1.70 (m, 2H). |
| B79 | mass calcd. for | ¹H NMR (400 MHZ, DMSO-d₆) δ 10.88 (s, 1H), 9.38 (d, J = |
| | C₄₅H₄₁N₇O₅S, 791.29; | 5.6 Hz, 2H), 8.66 (dd, J = 8.2, 2.0 Hz, 1H), 8.14 (d, J = |
| | m/z found, 792.29 | 8.2 Hz, 1H), 7.86 (d, J = 8.1 Hz, 1H), 7.72 (d, J = 14.9 Hz, |
| | [M + H]⁺. | 2H), 7.40-7.32 (m, 3H), 7.28 (s, 1H), 7.24 (d, J = 7.3 Hz, |
| | | 1H), 4.47-4.32 (m, 3H), 3.97 (d, J = 11.7 Hz, 2H), 3.78- |
| | | 3.67 (m, 4H), 3.63-3.54 (m, 1H), 3.50-3.38 (m, 2H), 3.17- |
| | | 3.04 (m, 2H), 3.00-2.87 (m, 1H), 2.80-2.66 (m, 1H), |
| | | 2.59-2.53 (m, 1H), 2.41-2.27 (m, 2H), 2.24-2.16 (m, |
| | | 1H), 2.08 (s, 3H), 2.07-1.97 (m, 1H), 1.85-1.68 (m, 4H). |
| B80 | mass calcd. for | ¹H NMR (400 MHZ, DMSO-d₆) δ 10.88 (s, 1H), 9.38 (s, |
| | C₄₅H₄₁N₇O₆, 775.8; m/z | 1H), 9.32-9.24 (m, 1H), 8.70-8.61 (m, 1H), 8.19-8.10 |
| | found, 776.2 [M + H]⁺. | (m, 1H), 7.99-7.94 (m, 1H), 7.71 (s, 1H), 7.60-7.54 (m, |
| | | 1H), 7.47 (s, 1H), 7.42-7.39 (m, 1H), 7.39-7.34 (m, 1H), |
| | | 7.30 (s, 1H), 6.49 (s, 1H), 4.39-4.33 (m, 2H), 4.18-4.13 |
| | | (m, 1H), 3.99-3.94 (m, 2H), 3.69 (s, 3H), 3.58-3.54 (m, |
| | | 2H), 3.21-3.14 (m, 2H), 3.09-2.99 (m, 2H), 2.97-2.92 |
| | | (m, 1H), 2.71-2.67 (m, 1H), 2.61-2.58 (m, 1H), 2.42 (s, |
| | | 3H), 2.36-2.31 (m, 1H), 2.24-2.18 (m, 2H), 2.11-2.05 |
| | | (m, 1H), 1.80-1.71 (m, 4H). |
| B81 | mass calcd. for | ¹H NMR (400 MHZ, DMSO-d₆) δ 10.91 (s, 1H), 9.43-9.34 |
| | C₄₄H₄₀N₈O₅S, 792.3; m/z | (m, 2H), 8.70-8.63 (m, 1H), 8.39 (d, J = 5.5 Hz, 1H), 8.14 |
| | found, 793.8 [M + H]⁺. | (d, J = 8.3 Hz, 1H), 7.96 (d, J = 5.8 Hz, 2H), 7.70 (s, 1H), |
| | | 7.41 (d, J = 7.2 Hz, 2H), 7.28 (s, 1H), 4.70-4.59 (m, 1H), |
| | | 4.42 (d, J = 5.8 Hz, 2H), 3.97 (d, J = 11.1 Hz, 2H), 3.75- |
| | | 3.68 (m, 4H), 3.64-3.56 (m, 1H), 3.50-3.39 (m, 2H), 3.16- |
| | | 3.09 (m, 2H), 3.00-2.92 (m, 1H), 2.62-2.53 (m, 2H), |
| | | 2.38-2.31 (m, 2H), 2.23-2.15 (m, 2H), 2.10 (s, 3H), 1.82- |
| | | 1.75 (m, 4H). |
| B82 | mass calcd. for | ¹H NMR (400 MHZ, DMSO-d₆) δ 10.87 (s, 1H), 9.44-9.31 |
| | C₄₅H₄₀FN₇O₆, 793.30; | (m, 2H), 8.67 (dd, J = 8.3, 2.0 Hz, 1H), 8.14 (d, J = 8.2 Hz, |
| | m/z found, 794 [M + H]⁺. | 1H), 7.75 (s, 1H), 7.53 (s, 1H), 7.48-7.41 (m, 3H), 7.37- |
| | | 7.32 (m, 1H), 7.29 (s, 1H), 7.12 (d, J = 7.4 Hz, 1H), 4.42 |
| | | (d, J = 5.8 Hz, 2H), 4.21 (dd, J = 12.3, 4.9 Hz, 1H), 3.89 |
| | | (dd, J = 11.3, 4.6 Hz, 2H), 3.75 (s, 3H), 3.72-3.68 (m, 2H), |
| | | 3.64-3.58 (m, 2H), 3.15-3.11 (m, 2H), 2.70-2.67 (m, |
| | | 1H), 2.55-2.53 (m, 1H), 2.37-2.22 (m, 5H), 2.12 (s, 3H), |
| | | 2.03-1.91 (m, 3H). |
| B83 | mass calcd. for | ¹H NMR (400 MHZ, DMSO-d₆) δ 10.88 (s, 1H), 9.09 (t, J = |
| | C₄₁H₃₄N₆O₆, 706; m/z | 5.9 Hz, 1H), 9.00 (d, J = 1.7 Hz, 1H), 8.25 (s, 1H), 7.96 |
| | found, 707 [M + H]⁺. | (s, 1H), 7.91 (s, 1H), 7.71 (s, 2H), 7.67 (d, J = 8.1 Hz, |
| | | 1H), 7.58 (d, J = 8.5 Hz, 1H), 7.53 (d, J = 8.6 Hz, 1H), |
| | | 7.42 (s, 1H), 7.36 (dd, J = 8.6, 1.6 Hz, 1H), 7.31 (d, J = |
| | | 7.7 Hz, 1H), 4.68 (t, J = 8.4 Hz, 1H), 4.52 (d, J = 11.1 Hz, |
| | | 1H), 4.32 (d, J = 5.8 Hz, 2H), 4.15 (dd, J = 12.3, 4.9 Hz, |
| | | 1H), 3.90-3.84 (m, 1H), 3.71 (s, 3H), 3.71-3.68 (m, |
| | | 1H), 2.77-2.67 (m, 1H), 2.62 (s, 3H), 2.58-2.56 (m, |
| | | 1H), 2.38-2.31 (m, 1H), 2.14 (s, 3H), 2.12-2.08 (m, |
| | | 1H). |
| B84 | mass calcd. for | ¹H NMR (400 MHZ, DMSO-d₆) δ 10.96 (s, 1H), 9.49 (t, J = |
| | C₄₄H₄₀N₈O₆, 776.2; m/z | 6.0 Hz, 1H), 9.24 (d, J = 2.1 Hz, 1H), 8.53 (dd, J = 8.3, |
| | found, 777.2 [M + H]⁺. | 2.2 Hz, 1H), 8.47 (d, J = 5.9 Hz, 1H), 8.23-7.98 (m, 2H), |
| | | 7.76-7.56 (m, 2H), 7.51 (s, 1H), 7.45 (s, 1H), 7.29 (s, 1H), |
| | | 4.45 (d, J = 6.4 Hz, 2H), 3.98-3.96 (m, 2H), 3.93-3.89 |
| | | (m, 1H), 3.74 (s, 3H), 3.54-3.38 (m, 2H), 3.12-3.03 (m, |
| | | 2H), 3.04-2.89 (m, 1H), 2.69-2.53 (m, 2H), 2.43-2.32 |
| | | (m, 2H), 2.26-2.14 (m, 4H), 2.08 (s, 3H), 1.88-1.71 (m, |
| | | 4H). |
| B85 | mass calcd. for | ¹H NMR (400 MHZ, DMSO-d₆) δ 10.92 (s, 1H), 9.37 (s, |
| | C₄₆H₄₃N₇O₆, 789.3; m/z | 1H), 9.31-9.25 (m, 1H), 8.68-8.61 (m, 1H), 8.14 (d, J = |
| | found, 790.3 [M + H]⁺. | 8.2 Hz, 1H), 8.01 (s, 1H), 7.71 (d, J = 5.7 Hz, 2H), 7.61- |
| | | 7.56 (m, 1H), 7.43-7.36 (m, 3H), 7.28 (s, 1H), 4.37 (d, J = |
| | | 5.6 Hz, 2H), 3.97 (d, J = 11.0 Hz, 2H), 3.75-3.71 (m, |
| | | 3H), 3.62-3.57 (m, 1H), 3.47-3.41 (m, 2H), 3.14-3.10 |
| | | (m, 2H), 3.01-2.90 (m, 2H), 2.72-2.66 (m, 1H), 2.59- |
| | | 2.53 (m, 1H), 2.39-2.25 (m, 2H), 2.23-2.17 (m, 1H), 2.10 |
| | | (s, 3H), 2.00-1.96 (m, 1H), 1.81-1.73 (m, 4H), 1.60 (s, |
| | | 3H). |
| B86 | mass calcd. for | ¹H NMR (400 MHz, DMSO-d₆) δ 10.88 (s, 1H), 9.39 (d, J = |
| | C₄₂H₃₇N₉O₅, 747.3; m/z | 1.5 Hz, 1H), 9.29 (t, J = 5.9 Hz, 1H), 8.68 (dd, J = 8.2, |
| | found, 748.5 [M + H]⁺. | 2.1 Hz, 1H), 8.15 (d, J = 8.2 Hz, 1H), 7.96 (s, 1H), 7.84 (s, |
| | | 1H), 7.71 (d, J = 1.2 Hz, 1H), 7.59-7.54 (m, 2H), 7.36 (dd, |
| | | J = 8.5, 1.6 Hz, 1H), 6.04 (s, 1H), 4.36 (d, J = 5.9 Hz, 2H), |
| | | 4.15 (dd, J = 12.2, 4.8 Hz, 1H), 3.99 (t, J = 7.4 Hz, 4H), |
| | | 3.73-3.67 (m, 2H), 3.55 (s, 3H), 3.09 (t, J = 6.4 Hz, 2H), |
| | | 2.75-2.66 (m, 1H), 2.59-2.52 (m, 1H), 2.37-2.29 (m, |
| | | 3H), 2.23-2.15 (m, 2H), 2.11-2.05 (m, 1H), 2.02 (s, 3H). |
| B87 | mass calcd. for | ¹H NMR (400 MHZ, DMSO-d₆) 10.88 (s, 1H), 9.54 (t, J = |
| | C₄₄H₄₀N₈O₆, 776.9; m/z | 5.9 Hz, 1H), 9.40 (d, J = 1.4 Hz, 1H), 9.24 (d, J = 1.4 Hz, |
| | found, 777.9 [M+H] . | 1H), 7.70 (d, J = 1.5 Hz, 1H), 7.50-7.45 (m, 1H), 7.43 (d, |
| | | J = 2.0 Hz, 2H), 7.34 (dd, J = 8.3, 7.4 Hz, 1H), 7.30 (dd, J = |
| | | 4.4, 1.2 Hz, 2H), 7.12 (d, J = 7.4 Hz, 1H), 4.44 (d, J = 5.8 |
| | | Hz, 2H), 4.21 (dd, J = 12.2, 4.9 Hz, 1H), 4.05-3.92 (m, |
| | | 4H), 3.73 (s, 3H), 3.67-3.60 (m, 1H), 3.52-3.32 (m, 2H), |
| | | 3.26-3.06 (m, 2H), 3.03-2.89 (m, 1H), 2.78-2.62 (m, |
| | | 1H), 2.58-2.50 (m, 2H), 2.39-2.29 (m, 2H), 2.10 (s, 3H), |
| | | 1.91-1.70 (m, 4H). |
| B88 | mass calcd. for | ¹H NMR (400 MHZ, DMSO-d₆) δ 10.89 (s, 1H), 9.47 (d, J = |
| | C₄₄H₃₆N₆O₆,744.27; m/z | 1.7 Hz, 1H), 9.34 (t, J = 5.9 Hz, 1H), 9.05 (s, 1H), 8.81 |
| | found, 745.27 [M + H]⁺. | (s, 1H), 8.77 (dd, J = 8.2, 2.1 Hz, 1H), 8.23 (t, J = 9.3 Hz, |
| | | 2H), 7.98 (dd, J = 13.4, 5.2 Hz, 2H), 7.74-7.68 (m, 2H), |
| | | 7.58 (d, J = 8.6 Hz, 1H), 7.37 (dd, J = 8.5, 1.5 Hz, 1H), 7.23 |
| | | (s, 1H), 6.87 (s, 1H), 5.30-5.17 (m, 1H), 4.39 (d, J = 5.9 |
| | | Hz, 2H), 4.14-4.08 (m, 1H), 3.64 (s, 3H), 2.75-2.63 (m, |
| | | 1H), 2.60-2.53 (m, 1H), 2.33-2.28 (m, 1H), 2.13-2.05 |
| | | (m, 1H), 1.93 (s, 3H), 1.30-1.20 (m, 6H). |
| B89 | mass calcd. for | ¹H NMR (400 MHZ, DMSO-d₆) δ 10.88 (s, 1H), 9.26 (t, J = |
| | C₄₄H₄₁N₉O₆, 791.8; m/z | 5.9 Hz, 1H), 9.21 (d, J = 2.0 Hz, 1H), 8.47-8.37 (m, 1H), |
| | found, 792.8 [M + H]⁺. | 7.87 (s, 1H), 7.58 (d, J = 1.4 Hz, 1H), 7.47 (d, J = 8.3 Hz, |
| | | 1H), 7.34 (dd, J = 8.4, 7.4 Hz, 1H), 7.30 (d, J = 1.0 Hz, 1H), |
| | | 7.12 (d, J = 7.4 Hz, 1H), 6.51 (s, 1H), 4.40 (d, J = 5.9 Hz, |
| | | 2H), 4.22 (dd, J = 12.2, 4.9 Hz, 1H), 3.80-3.72 (m, 2H), |
| | | 3.74-3.62 (m, 4H), 3.60 (s, 3H), 3.58-3.47 (m, 4H), 3.18- |
| | | 3.00 (m, 2H), 2.80-2.69 (m, 1H), 2.65 (s, 3H), 2.59- |
| | | 2.53 (m, 2H), 2.41-2.29 (m, 1H), 2.27-2.10 (m, 2H), 2.03 |
| | | (s, 3H). |
| B90 | mass calcd. for | ¹H NMR (400 MHZ, DMSO-d₆) δ 10.94 (s, 1H), 9.41 (t, J = |
| | C₄₅H₄₀FN₇O₆, 793.9; m/z | 6.0 Hz, 1H), 9.37 (d, J = 2.0 Hz, 1H), 8.66 (dd, J = 8.2, |
| | found, 794.9[M + H]⁺. | 2.1 Hz, 1H), 8.14 (d, J = 8.2 Hz, 1H), 7.70 (s, 1H), 7.57- |
| | | 7.51 (m, 1H), 7.41 (d, J = 6.9 Hz, 2H), 7.35 (d, J = 0.9 Hz, |
| | | 1H), 7.30-7.14 (m, 2H), 4.42 (d, J = 5.9 Hz, 2H), 4.36 (dd, |
| | | J = 7.9 Hz, 1H), 4.15-3.92 (m, 2H), 3.72 (s, 3H), 3.64- |
| | | 3.51 (m, 1H), 3.50-3.37 (m, 2H), 3.21-3.04 (m, 2H), 2.99- |
| | | 2.89 (m, 1H), 2.86-2.74 (m, 1H), 2.61-2.55 (m, 2H), |
| | | 2.36-2.27 (m, 2H), 2.25-2.15 (m, 1H), 2.10 (s, 3H), 2.04- |
| | | 1.92 (m, 1H), 1.90-1.59 (m, 4H). |
| B91 | mass calcd. for | ¹H NMR (400 MHZ, DMSO-d₆) δ 10.88 (s, 1H), 9.24 (t, J = |
| | C₄₄H₃₈FN₅O₆, 751.2; m/z | 5.8 Hz, 1H), 8.76 (s, 1H), 8.09 (s, 2H), 7.96 (s, 1H), 7.72 |
| | found, 752.2 [M+H] . | (d, J = 11.0 Hz, 2H), 7.57 (d, J = 8.5 Hz, 1H), 7.40-7.34 |
| | | (m, 2H), 7.25 (s, 1H), 7.18 (d, J = 7.6 Hz, 1H), 5.84 (d, J = |
| | | 12.9 Hz, 1H), 4.57-4.50 (m, 1H), 4.46-4.40 (m, 1H), |
| | | 4.36 (d, J = 5.8 Hz, 2H), 4.19-4.12 (m, 1H), 3.90-3.83 |
| | | (m, 1H), 3.72 (s, 3H), 3.67-3.63 (m, 1H), 3.11-3.04 (m, |
| | | 1H), 2.76-2.68 (m, 1H), 2.59-2.53 (m, 1H), 2.38-2.30 |
| | | (m, 1H), 2.12 (s, 3H), 2.10-2.03 (m, 1H), 1.32-1.27 (m, |
| | | 6H). |
| B92 | mass calcd. for | ¹H NMR (400 MHz, DMSO-d₆) δ 10.88 (s, 1H), 9.47 (s, |
| | C₄₅H₃₇N₇O₆, 771.3; m/z | 1H), 9.36-9.32 (m, 1H), 9.05 (s, 1H), 8.80 (s, 1H), 8.78- |
| | found, 772.3 [M + H]⁺. | 8.75 (m, 1H), 8.24-8.21 (m, 2H), 7.98-7.95 (m, 2H), 7.72 |
| | | (s, 1H), 7.71-7.68 (m, 1H), 7.60-7.57 (m, 1H), 7.39- |
| | | 7.36 (m, 1H), 7.14 (s, 1H), 6.71 (s, 1H), 4.40-4.37 (m, |
| | | 2H), 4.18-4.14 (m, 1H), 3.71 (s, 4H), 3.67 (s, 3H), 3.61 |
| | | (s, 4H), 2.74-2.69 (m, 1H), 2.58 (s, 1H), 2.35-2.31 (m, |
| | | 1H), 2.01-1.99 (m, 1H), 1.91 (s, 3H). |
| B93 | mass calcd. for | ¹H NMR (400 MHZ, DMSO-d₆) δ 10.88 (s, 1H), 9.47 (d, J = |
| | C₄₃H₃₄N₆O₅, 714.3; m/z | 1.9 Hz, 1H), 9.34 (t, J = 5.9 Hz, 1H), 8.98 (s, 1H), 8.82 |
| | found, 715.2 [M + H]⁺. | (s, 1H), 8.77 (dd, J = 8.2, 2.1 Hz, 1H), 8.26-8.20 (m, 2H), |
| | | 8.01-7.97 (m, 1H), 7.96 (s, 1H), 7.73-7.72 (m, 1H), 7.70 |
| | | (d, J = 7.1 Hz, 1H), 7.58 (d, J = 8.6 Hz, 1H), 7.51 (s, 1H), |
| | | 7.37 (dd, J = 8.6, 1.5 Hz, 1H), 7.33 (s, 1H), 4.38 (d, J = 5.9 |
| | | Hz, 2H), 4.15 (dd, J = 12.2, 4.8 Hz, 1H), 3.73 (s, 3H), 2.96- |
| | | 2.90 (m, 2H), 2.76-2.67 (m, 1H), 2.59-2.53 (m, 1H), |
| | | 2.38-2.30 (m, 1H), 2.12-2.06 (m, 1H), 1.98 (s, 3H), 1.33 |
| | | (t, J = 7.5 Hz, 3H). |
| B94 | mass calcd. for | ¹H NMR (400 MHZ, DMSO-d₆) δ 10.96 (s, 1H), 9.43 (t, J = |
| | C₄₄H₄₀N₈O₆, 776.3; m/z | 5.9 Hz, 1H), 9.37 (d, J = 1.6 Hz, 1H), 8.77-8.60 (m, 1H), |
| | found, 777.3 [M + H]⁺. | 8.47 (d, J = 5.9 Hz, 1H), 8.14 (d, J = 8.2 Hz, 1H), 7.70 (s, |
| | | 1H), 7.63 (d, J = 5.8 Hz, 1H), 7.52 (s, 1H), 7.42 (s, 1H), |
| | | 7.40 (s, 1H), 7.28 (s, 1H), 4.49-4.42 (m, 3H), 3.99-3.95 |
| | | (m, 2H), 3.72 (s, 3H), 3.48-3.38 (m, 4H), 3.15-3.09 (m, |
| | | 2H), 3.00-2.94 (m, 1H), 2.64-2.56 (m, 2H), 2.38-2.28 |
| | | (m, 2H), 2.24-2.16 (m, 2H), 2.10 (s, 3H), 1.82-1.74 (m, |
| | | 4H). |
| B95 | mass calcd. for | ¹H NMR (400 MHZ, DMSO-d₆) δ 10.88 (s, 1H), 9.53-9.41 |
| | C₄₄H₄₁N₇O₅, 747.3; m/z | (m, 1H), 9.13-9.06 (m, 1H), 8.51-8.42 (m, 1H), 8.23 (d, |
| | found, 748.2 [M + H]⁺. | J = 8.1 Hz, 1H), 7.75 (s, 1H), 7.46 (d, J = 8.3 Hz, 1H), 7.42 |
| | | (s, 1H), 7.36-7.32 (m, 2H), 7.28 (s, 2H), 7.12 (d, J = 7.6 |
| | | Hz, 1H), 6.68-6.65 (m, 1H), 4.44 (d, J = 5.9 Hz, 2H), 4.23- |
| | | 4.19 (m, 1H), 3.93-3.87 (m, 1H), 3.77-3.71 (m, 4H), |
| | | 3.68-3.64 (m, 1H), 3.38-3.38 (m, 6H), 3.12-3.06 (m, |
| | | 1H), 2.72-2.67 (m, 1H), 2.56-2.54 (m, 1H), 2.36-2.32 |
| | | (m, 1H), 2.11 (s, 3H), 2.05 (m, 1H), 1.30 (d, J = 6.9 Hz, |
| | | 6H). |
| B96 | mass calcd. for | ¹H NMR (400 MHZ, DMSO-d₆) δ 10.88 (s, 1H), 9.44 (t, J = |
| | C₄₄H₃₉N₇O₇, 777; m/z | 5.9 Hz, 1H), 9.35 (d, J = 1.7 Hz, 1H), 8.64 (dd, J = 8.2, |
| | found, 778 [M + H]⁺. | 2.1 Hz, 1H), 8.14 (d, J = 8.2 Hz, 1H), 7.87 (s, 1H), 7.46 (d, |
| | | J = 6.9 Hz, 2H), 7.40 (s, 1H), 7.34 (t, J = 7.9 Hz, 2H), 7.29 |
| | | (s, 1H), 7.12 (d, J = 7.4 Hz, 1H), 4.87-4.80 (m, 1H), 4.68 |
| | | (d, J = 11.2 Hz, 1H), 4.42 (d, J = 5.9 Hz, 2H), 4.21 (dd, J = |
| | | 12.2, 4.9 Hz, 1H), 4.01-3.93 (m, 3H), 3.75-3.69 (m, 4H), |
| | | 3.47-3.42 (m, 2H), 2.99-2.91 (m, 1H), 2.73-2.66 (m, |
| | | 1H), 2.56-2.53 (m, 1H), 2.38-2.29 (m, 1H), 2.11 (s, 3H), |
| | | 2.06-2.00 (m, 1H), 1.84-1.75 (m, 4H). |
| B97 | mass calcd. for | ¹H NMR (400 MHZ, DMSO-d₆) δ 10.83 (s, 1H), 9.50-9.32 |
| | C₄₆H₄₁N₇O₆, 787.3; m/z | (m, 2H), 8.66 (dd, J = 8.2, 1.7 Hz, 1H), 8.14 (d, J = 8.2 Hz, |
| | found, 788.3 [M + H]⁺. | 1H), 7.70 (s, 1H), 7.48 (d, J = 8.4 Hz, 1H), 7.41 (d, J = 7.4 |
| | | Hz, 2H), 7.36 (t, J = 7.9 Hz, 1H), 7.28 (s, 1H), 7.19 (s, 1H), |
| | | 6.97 (d, J = 7.4 Hz, 1H), 4.42 (d, J = 5.8 Hz, 2H), 3.97 (d, |
| | | J = 11.5 Hz, 2H), 3.72 (s, 3H), 3.62-3.58 (m, 1H), 3.48- |
| | | 3.42 (m, 4H), 3.15-3.10 (m, 1H), 3.02-2.99 (m, 2H), 2.77- |
| | | 2.71 (m, 2H), 2.36-2.30 (m, 3H), 2.25-2.18 (m, 1H), |
| | | 2.10 (s, 3H), 1.83-1.73 (m, 4H). |
| B98 | mass calcd. for | ¹H NMR (400 MHZ, DMSO-d₆) δ 10.88 (s, 1H), 9.40 (t, J = |
| | C₄₄H₃₉N₇O₇, 777.29; m/z | 5.9 Hz, 1H), 9.26 (d, J = 1.5 Hz, 1H), 8.55 (dd, J = 8.3, |
| | found, 778.29 [M + H]⁺. | 2.1 Hz, 1H), 8.11 (d, J = 8.2 Hz, 1H), 7.87 (s, 2H), 7.79 (s, |
| | | 1H), 7.46 (d, J = 8.3 Hz, 1H), 7.36-7.32 (m, 1H), 7.29 (s, |
| | | 1H), 7.18 (s, 1H), 7.12 (d, J = 7.4 Hz, 1H), 4.42-4.33 (m, |
| | | 2H), 4.21-4.11 (m, 1H), 3.96-3.89 (m, 2H), 3.88-3.80 |
| | | (m, 2H), 3.76 (s, 3H), 3.49-3.42 (m, 4H), 3.01-2.92 (m, |
| | | 1H), 2.69-2.61 (m, 1H), 2.54-2.47 (m, 1H), 2.34-2.29 |
| | | (m, 1H), 2.14 (s, 3H), 2.06-2.00 (m, 1H), 1.84-1.76 (m, |
| | | 4H). |
| B99 | mass calcd. for | ¹H NMR(400 MHZ, DMSO-d₆) δ 10.91 (s, 1H), 9.50 (d, J = |
| | C₄₂H₃₅N₇O₅, 717.3; m/z | 1.7 Hz, 1H), 9.41 (d, J = 7.2 Hz, 2H), 8.78 (d, J = 7.0 Hz, |
| | found, 718.3 [M + H]⁺. | 2H), 8.30 (s, 1H), 8.22 (d, J = 8.2 Hz, 1H), 8.09 (s, 1H), |
| | | 8.04 (d, J = 8.6 Hz, 1H), 7.92 (s, 1H), 7.69 (d, J = 6.9 Hz, |
| | | 1H), 7.49 (d, J = 8.6 Hz, 1H), 7.28 (s, 1H), 7.21 (s, 1H), |
| | | 4.41 (d, J = 5.9 Hz, 2H), 4.17 (dd, J = 12.4, 5.0 Hz, 1H), |
| | | 3.79-3.72 (m, 1H), 3.66 (s, 3H), 3.39 (s, 3H), 2.80-2.72 |
| | | (m, 1H), 2.61-2.55 (m, 1H), 2.45-2.35 (m, 1H), 2.17- |
| | | 2.08 (m, 1H), 1.35 (d, J = 6.8 Hz, 6H). |
| B100 | mass calcd. for | ¹H NMR (400 MHZ, DMSO-d₆) δ 10.88 (s, 1H), 9.24 (t, J = |
| | C₄₁H₃₁F₂N₅O₆, 727.2; | 6.0 Hz, 1H), 8.76 (d, J = 1.3 Hz, 1H), 8.09 (s, 2H), 7.96 |
| | m/z found, 728.2 | (s, 1H), 7.79 (s, 1H), 7.71 (d, J = 1.2 Hz, 1H), 7.57 (d, J = |
| | [M + H]⁺. | 8.5 Hz, 1H), 7.45 (dd, J = 11.3, 2.2 Hz, 1H), 7.33 (ddd, J = |
| | | 11.9, 9.0, 1.9 Hz, 2H), 7.20 (d, J = 7.6 Hz, 1H), 6.01 (d, J = |
| | | 12.7 Hz, 1H), 4.58-4.50 (m, 1H), 4.45-4.38 (m, 1H), |
| | | 4.38-4.31 (m, 2H), 4.18-4.12 (m, 1H), 3.88-3.81 (m, |
| | | 1H), 3.67 (s, 3H), 3.67-3.60 (m, 1H), 2.76-2.67 (m, 1H), |
| | | 2.60-2.53 (m, 1H), 2.39-2.28 (m, 1H), 2.12 (s, 3H), 2.11- |
| | | 2.04 (m, 1H). |
| B101 | mass calcd. for | ¹H NMR (400 MHZ, DMSO-d₆) δ 10.88 (s, 1H), 9.47 (d, J = |
| | C₄₅H₃₇N₇O₆, 771.3; m/z | 2.1 Hz, 1H), 9.34 (t, J = 6.0 Hz, 1H), 9.02 (s, 1H), 8.80 |
| | found, 772.3 [M + H]⁺. | (s, 1H), 8.77 (dd, J = 8.3, 2.2 Hz, 1H), 8.22 (d, J = 8.1 Hz, |
| | | 2H), 7.96 (d, J = 4.2 Hz, 2H), 7.72 (m, 1H), 7.70-7.46 (m, |
| | | 2H), 7.37 (dd, J = 8.5, 1.6 Hz, 1H), 7.09 (s, 1H), 6.28 (s, |
| | | 1H), 5.63 (s, 1H), 4.38 (d, J = 6.0 Hz, 2H), 4.15 (dd, J = |
| | | 12.2, 4.8 Hz, 1H), 4.00-3.86 (m, 4H), 3.62 (s, 3H), 2.79- |
| | | 2.64 (m, 2H), 2.15-2.03 (m, 2H), 1.88 (s, 3H), 1.45 (s, |
| | | 3H). |
| B102 | mass calcd. for | ¹H NMR (400 MHZ, DMSO-d₆) δ 10.88 (s, 1H), 9.47 (s, |
| | C₄₄H₃₆N₆O₅, 728.3; m/z | 1H), 9.33 (t, J = 5.9 Hz, 1H), 9.00 (s, 1H), 8.82 (s, 1H), 8.77 |
| | found, 729.3 [M + H]⁺. | (dd, J = 8.3, 2.0 Hz, 1H), 8.23 (dd, J = 13.4, 8.3 Hz, 2H), |
| | | 7.98 (dd, J = 14.2, 6.2 Hz, 2H), 7.71 (d, J = 7.0 Hz, 2H), |
| | | 7.58 (d, J = 8.5 Hz, 1H), 7.47 (s, 1H), 7.39-7.35 (m, 2H), |
| | | 4.38 (d, J = 5.9 Hz, 2H), 4.15 (dd, J = 12.2, 4.8 Hz, 1H), |
| | | 3.74 (s, 3H), 3.25-3.20 (m, 1H), 2.76-2.67 (m, 1H), 2.59- |
| | | 2.52 (m, 1H), 2.38-2.30 (m, 1H), 2.12-2.04 (m, 1H), |
| | | 1.99 (s, 3H), 1.36-1.31 (m, 6H). |
| B103 | mass calcd. for | ¹H NMR (400 MHZ, DMSO-d₆) δ 10.88 (s, 1H), 9.26 (t, J = |
| | C₄₅H₄₂N₈O₆, 790.32; m/z | 5.8 Hz, 1H), 9.22 (s, 1H), 8.45 (s, 1H), 7.79 (d, J = 4.9 |
| | found, 791.32 [M + H]⁺. | Hz, 1H), 7.74 (s, 1H), 7.47 (d, J = 8.3 Hz, 1H), 7.34 (t, J = |
| | | 7.9 Hz, 1H), 7.28 (d, J = 12.3 Hz, 2H), 7.12 (d, J = 7.4 Hz, |
| | | 1H), 4.40 (d, J = 5.7 Hz, 2H), 4.22 (dd,J = 12.1, 4.7 Hz, |
| | | 1H), 3.97-3.87 (m, 2H), 3.74-3.56 (m, 5H), 3.44 (s, 3H), |
| | | 3.11-3.08 (m, 2H), 3.06-2.98 (m, 1H), 2.76-2.62 (m, |
| | | 5H), 2.55-2.50 (m, 1H), 2.38-2.30 (m, 1H), 2.12 (s, 3H), |
| | | 2.04-1.97 (m, 1H), 1.85-1.80 (m, 4H). |
| B104 | mass calcd. for | ¹H NMR (400 MHZ, DMSO-d₆) δ 10.88 (s, 1H), 9.41 (t, J = |
| | C₄₁H₃₆N₈O₅, 720.2; m/z | 6.0 Hz, 1H), 9.36 (s, 1H), 8.71-8.63 (m, 1H), 8.15 (d, J = |
| | found, 721.2 [M + H]⁺. | 8.2 Hz, 1H), 7.75 (s, 1H), 7.46 (d, J = 8.4 Hz, 1H), 7.38 |
| | | (s, 1H), 7.36-7.32 (m, 1H), 7.29 (s, 1H), 7.17 (s, 1H), 7.12 |
| | | (d, J = 7.4 Hz, 1H), 6.83 (s, 1H), 4.43 (d, J = 5.9 Hz, 2H), |
| | | 4.23-4.19 (m, 1H), 4.04-3.94 (m, 2H), 3.92-3.84 (m, |
| | | 2H), 3.69-3.68 (m, 3H), 3.37 (s, 3H), 2.68 (d, J = 7.3 Hz, |
| | | 1H), 2.55-2.54 (m, 1H), 2.49-2.48 (m, 3H), 2.35-2.32 |
| | | (m, 1H), 2.08 (s, 3H), 2.05-2.01 (m, 1H). |
| B105 | mass calcd. for | ¹H NMR (400 MHZ, DMSO-d₆) δ 10.87 (s, 1H), 9.37 (d, J = |
| | C₄₄H₃₉N₇O₆, 761.30; m/z | 1.4 Hz, 1H), 9.28 (t, J = 5.9 Hz, 1H), 8.68-8.64 (m, 1H), |
| | found, 762 [M + H]⁺. | 8.13 (d, J = 8.5 Hz, 1H), 7.95 (s, 1H), 7.81 (d, J = 9.7 Hz, |
| | | 1H), 7.71 (d, J = 1.3 Hz, 1H), 7.57 (d, J = 8.6 Hz, 1H), 7.46- |
| | | 7.43 (m, 2H), 7.36 (dd, J = 8.5, 1.6 Hz, 1H), 7.31 (s, 1H), |
| | | 6.60-6.56 (m, 1H), 4.39-4.32 (m, 2H), 4.15 (dd, J = 12.2, |
| | | 4.9 Hz, 1H), 3.97 (d, J = 11.6 Hz, 2H), 3.78-3.71 (m, 1H), |
| | | 3.63-3.57 (m, 1H), 3.49-3.43 (m, 2H), 3.28 (s, 3H), 3.16- |
| | | 3.08 (m, 2H), 3.02-2.93 (m, 1H), 2.74-2.67 (m, 1H), |
| | | 2.61-2.53 (m, 1H), 2.37-2.27 (m, 2H), 2.25-2.17 (m, |
| | | 1H), 2.10-2.04 (m, 1H), 1.84-1.76 (m, 4H). |
| B106 | mass calcd. for | ¹H NMR (400 MHZ, DMSO-d₆) δ 10.95 (s, 1H), 9.43 (t, J = |
| | C₃₉H₃₂N₈O₅, 692.3; m/z | 5.9 Hz, 1H), 9.37 (d, J = 1.5 Hz, 1H), 8.66 (dd, J = 8.2, |
| | found, 693.2 [M + H]⁺. | 2.1 Hz, 1H), 8.45 (d, J = 5.8 Hz, 1H), 8.14 (d, J = 8.4 Hz, |
| | | 1H), 7.75 (s, 1H), 7.73-7.68 (m, 1H), 7.62-7.56 (m, 2H), |
| | | 7.52-7.50 (m, 1H), 7.41 (s, 1H), 7.31 (d, J = 7.2 Hz, 1H), |
| | | 4.48-4.43 (m, 3H), 3.73-3.70 (m, 4H), 3.63-3.60 (m, |
| | | 1H), 3.17-3.11 (m, 2H), 2.64-2.53 (m, 2H), 2.39-2.31 |
| | | (m, 2H), 2.24-2.15 (m, 2H), 2.13-2.11 (m, 3H). |
| B107 | mass calcd. for | ¹H NMR (400 MHZ, DMSO-d₆) δ 10.84 (d, J = 1.5 Hz, 1H), |
| | C₄₆H₄₁N₇O₆, 787.3; m/z | 9.36 (d, J = 1.4 Hz, 1H), 9.26 (t, J = 5.9 Hz, 1H), 8.69- |
| | found, 788.3 [M + H]⁺. | 8.62 (m, 1H), 8.13 (d, J = 8.1 Hz, 1H), 7.95 (s, 1H), 7.70 (s, |
| | | 1H), 7.57 (d, J = 8.0 Hz, 2H), 7.42 (s, 1H), 7.40 (s, 1H), |
| | | 7.38-7.34 (m, 1H), 7.28 (s, 1H), 4.35 (d, J = 5.8 Hz, 2H), |
| | | 3.99-3.95 (m, 2H), 3.72 (s, 3H), 3.72-3.67 (m, 1H), 3.62- |
| | | 3.57 (m, 1H), 3.46-3.42 (m, 2H), 3.16-3.11 (m, 2H), |
| | | 3.04-3.00 (m, 1H), 2.98-2.95 (m, 1H), 2.91-2.88 (m, |
| | | 2H), 2.78-2.72 (m, 2H), 2.36-2.31 (m, 1H), 2.25-2.21 |
| | | (m, 1H), 2.10 (s, 3H), 1.81-1.76 (m, 4H). |
| B108 | mass calcd. for | ¹H NMR(400 MHZ, DMSO-d₆) δ 11.07 (s, 1H), 9.37 (d, J = |
| | C₄₄H₄₀N₈O₆, 776.31; m/z | 1.4 Hz, 1H), 9.30 (t, J = 5.8 Hz, 1H), 8.66 (dd, J = 8.2, |
| | found, 777.31 [M + H]⁺. | 2.1 Hz, 1H), 8.13 (d, J = 8.1 Hz, 1H), 8.02 (s, 1H), 7.76 |
| | | (d, J = 8.8 Hz, 1H), 7.72-7.62 (m, 2H), 7.41 (d, J = 7.8 |
| | | Hz, 2H), 7.28 (s, 1H), 4.60 (dd, J = 12.0, 4.9 Hz, 1H), 4.36 |
| | | (d, J = 5.8 Hz, 2H), 3.97-3.87 (m, 2H), 3.72-3.60 (m, |
| | | 4H), 3.60-3.50 (m, 1H), 3.45-3.32 (m, 2H), 3.12-3.01 |
| | | (m, 2H), 2.96-2.86 (m, 1H), 2.79-2.65 (m, 1H), 2.62- |
| | | 2.52 (m, 2H), 2.33-2.22 (m, 1H), 2.18-2.11 (m, 2H), |
| | | 2.10 (s, 3H), 1.78-1.67 (m, 4H). |
| B109 | LC-MS (ESI): mass | ¹H NMR (400 MHZ, DMSO-d₆) δ 10.88 (s, 1H), 9.26-9.19 |
| | calcd. for C₄₅H₄₀N₆O₇, | (m, 2H), 8.50 (dd, J = 8.3, 2.2 Hz, 1H), 8.08 (d, J = 8.3 Hz, |
| | 766.3; m/z found, 767.3 | 1H), 7.96 (s, 1H), 7.84 (s, 1H), 7.71 (s, 2H), 7.57 (d, J = 8.6 |
| | [M+H] . | Hz, 1H), 7.43 (s, 1H), 7.39-7.34 (m, 2H), 7.29-7.26 (m, |
| | | 1H), 4.69-4.63 (m, 1H), 4.55-4.49 (m, 1H), 4.35 (d, J = |
| | | 6.0 Hz, 2H), 4.15 (dd, J = 12.3, 4.9 Hz, 1H), 3.99-3.94 |
| | | (m, 2H), 3.92-3.86 (m, 1H), 3.74-3.68 (m, 4H), 3.48- |
| | | 3.42 (m, 2H), 2.99-2.93 (m, 1H), 2.75-2.68 (m, 1H), 2.59- |
| | | 2.54 (m, 1H), 2.38-2.29 (m, 1H), 2.13-2.11 (m, 3H), |
| | | 2.05-1.97 (m, 1H), 1.85-1.76 (m, 4H). |
| B110 | mass calcd. for | ¹H NMR (400 MHZ, DMSO-d₆) δ 10.88 (s, 1H), 9.36 (d, J = |
| | C₄₀H₃₃N₇O₅, 691.3; m/z | 1.7 Hz, 1H), 9.28 (t, J = 6.0 Hz, 1H), 8.65 (dd, J = 8.2, |
| | found, 692.4 [M + H]⁺. | 2.0 Hz, 1H), 8.13 (d, J = 8.2 Hz, 1H), 7.95 (s, 1H), 7.75 (s, |
| | | 1H), 7.70 (t, J = 8.1 Hz, 2H), 7.57 (d, J = 8.6 Hz, 2H), 7.41 |
| | | (s, 1H), 7.36 (dd, J = 8.5, 1.5 Hz, 1H), 7.30 (d, J = 7.6 Hz, |
| | | 1H), 4.35 (d, J = 5.9 Hz, 2H), 4.15 (dd, J = 12.2, 4.8 Hz, |
| | | 1H), 3.68 (d, J = 3.3 Hz, 4H), 3.61 (dd, J = 10.7, 5.6 Hz, |
| | | 1H), 3.16-3.09 (m, 2H), 2.75-2.67 (m, 1H), 2.59-2.53 |
| | | (m, 1H), 2.38-2.28 (m, 2H), 2.24-2.18 (m, 1H), 2.14- |
| | | 2.07 (m, 4H). |
| B111 | mass calcd. for | ¹H NMR (400 MHZ, DMSO-d₆) δ 11.07 (s, 1H), 9.49 (d, J = |
| | C₄₂H₃₄F2N₈O₄, 752.3; | 1.6 Hz, 1H), 9.39 (t, J = 5.9 Hz, 1H), 8.93 (s, 1H), 8.81- |
| | m/z found, 753.4 | 8.76 (m, 2H), 8.23 (d, J = 8.2 Hz, 1H), 8.19 (d, J = 8.4 |
| | [M + H]⁺. | Hz, 1H), 8.15 (s, 1H), 7.98-7.92 (m, 1H), 7.82 (s, 1H), |
| | | 7.78 (d, J = 8.3 Hz, 1H), 7.63 (d, J = 6.8 Hz, 1H), 7.22 (s, |
| | | 1H), 7.18 (d, J = 9.3 Hz, 2H), 5.94-5.86 (m, 1H), 4.41 |
| | | (d, J = 5.9 Hz, 2H), 4.17 (d, J = 14.9 Hz, 1H), 3.93 (d, J = |
| | | 14.9 Hz, 1H), 3.35 (s, 3H), 2.76 (dd, J = 12.1, 9.0 Hz, 2H), |
| | | 2.70 (s, 3H), 2.28-2.22 (m, 1H), 2.06 (dd, J = 23.6, 14.4 |
| | | Hz, 4H). |
| B112 | mass calcd. for | ¹H NMR (400 MHZ, DMSO-d₆) δ 10.87 (s, 1H), 9.27-9.19 |
| | C₄₁H₃₄N₆O₇S, 722.2; m/z | (m, 2H), 8.52-8.46 (m, 1H), 8.07 (d, J = 8.2 Hz, 1H), 7.95 |
| | found, 723.3 [M+H]+ | (s, 1H), 7.78 (s, 1H), 7.73-7.68 (m, 2H), 7.57 (d, J = 8.5 |
| | | Hz, 1H), 7.46 (s, 1H), 7.37-7.33 (m, 1H), 6.97 (s, 2H), |
| | | 4.72-4.45 (m, 2H), 4.34 (d, J = 5.9 Hz, 2H), 4.15 (dd, J = |
| | | 12.2, 4.9 Hz, 1H), 3.92-3.86 (m, 4H), 3.72-3.65 (m, 4H), |
| | | 2.72-2.66 (m, 1H), 2.58-2.54 (m, 1H), 2.35-2.30 (m, |
| | | 1H), 2.11-2.05 (m, 4H). |
| B113 | mass calcd. for | ¹H NMR (400 MHZ, DMSO-d₆) δ 10.87 (s, 1H), 9.23 (t, J = |
| | C₄₅H₃₉FN₆O₇, 794.3; m/z | 6.0 Hz, 1H), 8.87-8.64 (m, 1H), 8.08 (d, J = 1.5 Hz, 2H), |
| | found, 795.3 [M + H]⁺. | 7.95 (s, 1H), 7.70 (d, J = 1.6 Hz, 1H), 7.66-7.53 (m, 2H), |
| | | 7.36 (dd, J = 8.6, 1.7 Hz, 1H), 7.16 (d, J = 7.6 Hz, 1H), 6.46 |
| | | (d, J = 2.0 Hz, 1H), 6.30 (d, J = 2.0 Hz, 1H), 5.90 (d, J = |
| | | 12.9 Hz, 1H), 4.53-4.28 (m, 6H), 4.24-4.09 (m, 3H), |
| | | 3.97-3.75 (m, 3H), 3.63 (s, 3H), 3.26 (s, 3H), 2.78-2.55 |
| | | (m, 2H), 2.40-2.28 (m, 2H), 2.08-1.98 (m, 3H). |
| B114 | mass calcd. for | ¹H NMR (400 MHZ, DMSO-d4) 8 10.88 (s, 1H), 9.41-9.31 |
| | C₄₁H₃₅N₇O₅, 705.3; m/z | (m, 1H), 9.18 (s, 1H), 8.53-8.45 (m, 1H), 8.14-8.09 (m, |
| | found, 706.3 [M + H]⁺. | 1H), 7.80 (s, 1H), 7.71-7.66 (m, 1H), 7.55-7.50 (m, 1H), |
| | | 7.47-7.43 (m, 1H), 7.36-7.32 (m, 1H), 7.29-7.23 (m, |
| | | 3H), 7.13-7.09 (m, 1H), 6.96 (s, 1H), 4.46-4.39 (m, 2H), |
| | | 4.23-4.18 (m, 1H), 3.88-3.81 (m, 2H), 3.71 (s, 3H), 3.65- |
| | | 3.58 (m, 2H), 3.20 (s, 3H), 2.72-2.67 (m, 1H), 2.56- |
| | | 2.54 (m, 1H), 2.36-2.31 (m, 1H), 2.11 (s, 3H), 2.05-2.00 |
| | | (m, 1H). |
| B115 | mass calcd. for | ¹H NMR (400 MHZ, DMSO-d₆) δ 10.88 (s, 1H), 9.40 (d, J = |
| | C₄₅H₄₁N₉O₆, 803.3; m/z | 1.4 Hz, 1H), 9.30 (t, J = 6.0 Hz, 1H), 8.69 (dd, J = 8.2, |
| | found, 804.8 [M + H]⁺. | 2.1 Hz, 1H), 8.15 (d, J = 8.2 Hz, 1H), 7.96 (s, 1H), 7.87 (s, |
| | | 1H), 7.71 (d, J = 1.3 Hz, 1H), 7.57 (d, J = 8.9 Hz, 2H), 7.36 |
| | | (dd, J = 8.6, 1.6 Hz, 1H), 6.40 (s, 1H), 4.44-4.41 (m, 2H), |
| | | 4.36 (d, J = 5.8 Hz, 2H), 4.18-4.12 (m, 1H), 3.95-3.90 |
| | | (m, 2H), 3.76-3.71 (m, 2H), 3.59 (s, 3H), 3.11-3.08 (m, |
| | | 2H), 3.02 (s, 1H), 2.99 (s, 1H), 2.73-2.67 (m, 1H), 2.59- |
| | | 2.54 (m, 1H), 2.36-2.31 (m, 1H), 2.19 (s, 2H), 2.11-2.06 |
| | | (m, 1H), 2.02 (s, 3H), 1.85-1.79 (m, 2H), 1.75-1.69 (m, |
| | | 2H). |
| B116 | mass calcd. for | ¹HNMR(400 MHZ, DMSO-d₆) δ 10.91-10.87 (m, 1H), |
| | C₄₆H₄₃N₇O₆, 789.3; m/z | 9.35-9.28 (m, 1H), 9.14-9.05 (m, 1H), 8.61 (d, J = 8.2 |
| | found, 790.1 [M + H]⁺. | Hz, 1H), 8.10-8.05 (m, 1H), 7.87 (s, 1H), 7.70 (s, 1H), |
| | | 7.52-7.48 (m, 2H), 7.41 (s, 1H), 7.38 (s, 1H), 7.32-7.27 |
| | | (m, 2H), 6.50-6.44 (m, 1H), 5.81-5.75 (m, 1H), 4.13- |
| | | 4.03 (m, 3H), 3.97 (d, J = 11.2 Hz, 2H), 3.72 (s, 3H), 3.61- |
| | | 3.57 (m, 2H), 3.13-3.09 (m, 2H), 3.00-2.91 (m, 2H), |
| | | 2.74-2.66 (m, 2H), 2.60-2.58 (m, 1H), 2.36-2.27 (m, |
| | | 3H), 2.23-2.19 (m, 1H), 2.10 (s, 3H), 1.80-1.75 (m, 4H). |
| B117 | mass calcd. for | ¹HNMR (400 MHZ, DMSO-d₆) δ 10.87 (s, 1H), 9.40-9.33 |
| | C₄₅H₃₉D₂N₇O₆, 777.3; | (m, 1H), 9.26 (s, 1H), 8.68-8.63 (m, 1H), 8.13 (d, J = 8.4 |
| | m/z found, 778.3 | Hz, 1H), 7.95 (s, 1H), 7.70 (s, 2H), 7.58-7.55 (m, 1H), |
| | [M+H] . | 7.43-7.39 (m, 2H), 7.37-7.34 (m, 1H), 7.28 (s, 1H), 4.16- |
| | | 4.14 (m, 1H), 3.97 (d, J = 11.5 Hz, 2H), 3.74-3.69 (m, |
| | | 4H), 3.62-3.57 (m, 1H), 3.48-3.41 (m, 2H), 3.16-3.09 |
| | | (m, 2H), 2.99-2.92 (m, 1H), 2.74-2.67 (m, 1H), 2.59- |
| | | 2.53 (m, 1H), 2.37-2.29 (m, 2H), 2.22 (d, J = 4.0 Hz, 1H), |
| | | 2.11-2.06 (m, 4H), 1.82-1.75 (m, 4H). |
| B118 | mass calcd. for | ¹H NMR (400 MHZ, DMSO-d₆) δ 10.88 (s, 1H), 9.48-9.38 |
| | C₄₀H₃₄N₈O₅ Exact Mass: | (m, 2H), 8.74-8.70 (m, 1H), 8.16 (d, J = 8.2 Hz, 1H), 7.81 |
| | 706.2; m/z found, 707.2 | (s, 1H), 7.73 (d, J = 1.0 Hz, 1H), 7.46 (d, J = 8.3 Hz, 1H), |
| | [M + H]⁺. | 7.37-7.32 (m, 1H), 7.29 (s, 1H), 7.21 (s, 1H), 7.12 (d, J = |
| | | 7.3 Hz, 1H), 4.43 (d, J = 5.9 Hz, 2H), 4.23-4.19 (m, 1H), |
| | | 3.74 (s, 3H), 3.72 (s, 2H), 3.12 (t, J = 6.2 Hz, 2H), 2.75- |
| | | 2.66 (m, 2H), 2.57 (s, 3H), 2.55-2.53 (m, 1H), 2.37-2.28 |
| | | (m, 2H), 2.11 (s, 3H), 2.06-2.00 (m, 1H). |
| B119 | mass calcd. for | ¹H NMR (400 MHZ, DMSO-d₆) δ 10.88 (s, 1H), 9.38 (d, J = |
| | C₄₀H₃₂ClN₇O₅, 725.3; | 1.5 Hz, 1H), 9.28 (t, J = 5.9 Hz, 1H), 8.67 (dd, J = 8.2, |
| | m/z found, 726.1 | 2.0 Hz, 1H), 8.14 (d, J = 8.2 Hz, 1H), 7.95 (s, 1H), 7.74 (s, |
| | [M + H]⁺. | 1H), 7.71 (s, 1H), 7.65 (s, 1H), 7.57 (d, J = 8.5 Hz, 1H), |
| | | 7.51 (s, 1H), 7.45 (d, J = 1.7 Hz, 1H), 7.36 (dd, J = 8.5, 1.4 |
| | | Hz, 1H), 4.35 (d, J = 5.8 Hz, 2H), 4.18-4.12 (m, 1H), 3.77- |
| | | 3.71 (m, 1H), 3.69 (s, 3H), 3.62-3.57 (m, 1H), 3.14- |
| | | 3.07 (m, 2H), 2.74-2.67 (m, 1H), 2.58-2.53 (m, 1H), 2.36- |
| | | 2.28 (m, 2H), 2.24-2.18 (m, 1H), 2.10 (s, 3H), 2.08- |
| | | 2.00 (m, 1H). |
| B120 | mass calcd. for | ¹H NMR (400 MHZ, DMSO-d₆) δ 10.96 (s, 1H), 9.46-9.39 |
| | C₄₀H₃₄N₈O₅, 706.2; m/z | (m, 1H), 9.39-9.35 (m, 1H), 8.66 (dd, J = 8.2, 2.1 Hz, 1H), |
| | found, 707.1 [M + H]⁺. | 8.46 (d, J = 5.9 Hz, 1H), 8.14 (d, J = 8.4 Hz, 1H), 7.69 (s, |
| | | 1H), 7.62 (d, J = 5.9 Hz, 1H), 7.51 (s, 1H), 7.43-7.40 (m, |
| | | 2H), 7.15 (s, 1H), 4.49-4.42 (m, 3H), 3.70-3.68 (m, 5H), |
| | | 3.63-3.57 (m, 2H), 3.15-3.09 (m, 2H), 2.66-2.53 (m, |
| | | 2H), 2.48 (s, 3H), 2.34 (dd, J = 5.3, 3.5 Hz, 1H), 2.21-2.18 |
| | | (m, 1H), 2.09 (s, 3H). |
| B121 | mass calcd. for | ¹H NMR (400 MHZ, DMSO-d₆) δ 10.88 (s, 1H), 9.40 (d, J = |
| | C₄₁H₃₆N₈O₅, 720.1; m/z | 1.9 Hz, 1H), 9.30 (t, J = 5.9 Hz, 1H), 8.70 (dd, J = 8.2, |
| | found, 721.3 [M + H]⁺. | 2.0 Hz, 1H), 8.15 (d, J = 8.2 Hz, 1H), 7.96 (s, 1H), 7.83 (s, |
| | | 1H), 7.76 (s, 1H), 7.72 (s, 1H), 7.57 (d, J = 8.5 Hz, 1H), |
| | | 7.39-7.35 (m, 1H), 7.31 (s, 1H), 4.36 (d, J = 5.9 Hz, 2H), |
| | | 4.18-4.12 (m, 1H), 3.78-3.72 (m, 2H), 3.66 (s, 3H), 3.14- |
| | | 3.09 (m, 2H), 2.82-2.76 (m, 2H), 2.73-2.66 (m, 1H), |
| | | 2.60-2.54 (m, 1H), 2.38-2.29 (m, 1H), 2.25-2.17 (m, |
| | | 2H), 2.12-2.10 (m, 3H), 2.10-2.05 (m, 1H), 1.27-1.22 |
| | | (m, 3H). |
| B122 | mass calcd. for | ¹H NMR (400 MHZ, DMSO-d₆) δ 10.88 (s, 1H), 9.37 (d, J = |
| | C₄₆H₄₃N₇O₆, 789.3; m/z | 2.0 Hz, 1H), 9.27 (t, J = 6.0 Hz, 1H), 8.66 (dd, J = 8.2, |
| | found, 790.3 [M+H]. | 2.1 Hz, 1H), 8.13 (d, J = 8.3 Hz, 1H), 7.95 (s, 1H), 7.71 (d, |
| | | J = 1.6 Hz, 1H), 7.62-7.53 (m, 2H), 7.45 (s, 1H), 7.41 (s, |
| | | 1H), 7.39-7.35 (m, 1H), 7.29 (d, J = 1.5 Hz, 1H), 4.35 (d, |
| | | J = 5.9 Hz, 2H), 4.15 (dd, J = 12.2, 4.9 Hz, 1H), 4.07-3.91 |
| | | (m, 2H), 3.72 (s, 3H), 3.67-3.58 (m, 2H), 3.50-3.40 (m, |
| | | 2H), 3.17-3.10 (m, 2H), 3.02-2.90 (m, 1H), 2.79-2.69 |
| | | (m, 2H), 2.61-2.55 (m, 2H), 2.37-2.26 (m, 2H), 2.25- |
| | | 2.19 (m, 1H), 2.10-2.00 (m, 1H), 1.95-1.69 (m, 4H), 1.07 |
| | | (t, J = 7.4 Hz, 3H). |
| B123 | mass calcd. for | ¹H NMR (400 MHZ, DMSO-d₆) δ 10.88 (s, 1H), 9.24 (s, |
| | C₄₀H₃₁FN₆O₆, 710.2; m/z | 1H), 8.76 (s, 1H), 8.03 (d, J = 50.7 Hz, 3H), 7.64 (d, J = |
| | found, 711.2 [M + H]⁺. | 54.6 Hz, 3H), 7.36 (s, 1H), 7.19 (d, J = 7.2 Hz, 1H), 7.03- |
| | | 6.95 (m, 2H), 5.91 (s, 1H), 4.39 (d, J = 35.1 Hz, 4H), 4.16 |
| | | (dd, J = 11.2, 5.9 Hz, 1H), 3.95-3.84 (m, 2H), 3.72-3.62 |
| | | (m, 3H), 2.13-2.05 (m, 4H), 1.33-1.18 (m, 3H). |
| B124 | mass calcd. for | ¹H NMR (400 MHZ, DMSO-d₆) δ 10.88 (s, 1H), 9.31 (dd, |
| | C₄₅H₄₂FN₈O₆, 790.2; m/z | J = 13.9, 8.1 Hz, 2H), 8.67 (dd, J = 8.2, 2.1 Hz, 1H), 8.17 |
| | found, 791.2 [M + H]⁺. | (d, J = 8.1 Hz, 1H), 7.96 (s, 1H), 7.77 (s, 1H), 7.70 (s, 1H), |
| | | 7.57 (d, J = 8.6 Hz, 1H), 7.40 (s, 1H), 7.36 (dd, J = 8.5, 1.5 |
| | | Hz, 1H), 7.29 (s, 1H), 6.79 (s, 1H), 4.36 (d, J = 5.9 Hz, 2H), |
| | | 4.15 (dd, J = 12.2, 4.8 Hz, 1H), 3.96 (s, 2H), 3.73 (d, J = |
| | | 12.8 Hz, 5H), 3.64-3.57 (m, 2H), 3.46 (d, J = 2.9 Hz, 2H), |
| | | 3.41 (s, 3H), 3.00-2.94 (m, 1H), 2.74-2.66 (m, 1H), 2.61- |
| | | 2.53 (m, 1H), 2.36-2.29 (m, 1H), 2.09 (s, 3H), 2.08- |
| | | 2.04 (m, 1H), 1.83-1.76 (m, 4H). |
| B125 | mass calcd. for | ¹HNMR(400 MHZ, DMSO-d₆) δ 10.87 (s, 1H), 9.37 (d, J = |
| | C₄₃H₃₉N₇O₅, 733.83; m/z | 1.9 Hz, 1H), 9.27 (t, J = 5.9 Hz, 1H), 8.66 (dd, J = 8.2, 2.0 |
| | found, 734.4 [M + H]⁺. | Hz, 1H), 8.13 (d, J = 8.2 Hz, 1H), 7.95 (s, 1H), 7.70 (d, J = |
| | | 7.1 Hz, 2H), 7.57 (d, J = 8.6 Hz, 1H), 7.40-7.34 (m, 3H), |
| | | 7.25 (s, 1H), 4.35 (d, J = 6.0 Hz, 2H), 4.15 (dd, J = 12.3, |
| | | 4.8 Hz, 1H), 3.74-3.69 (m, 4H), 3.63-3.56 (m, 1H), 3.15- |
| | | 3.11 (m, 2H), 3.10-3.05 (m, 1H), 2.73-2.67 (m, 1H), |
| | | 2.59-2.54 (m, 1H), 2.37-2.30 (m, 2H), 2.24-2.17 (m, |
| | | 1H), 2.10 (s, 3H), 2.09-2.03 (m, 1H), 1.29 (dd, J = 6.9, |
| | | 2.1 Hz, 6H). |
| B126 | mass calcd. for | ¹H NMR (400 MHZ, DMSO-d₆) δ 10.88 (s, 1H), 9.41 (t, J = |
| | C₄₀H₃₂ClN₇O₅, 725.22; | 6.0 Hz, 1H), 9.39-9.35 (m, 1H), 8.67 (dd, J = 8.2, 2.1 |
| | m/z found, 726.22 | Hz, 1H), 8.14 (d, J = 8.3 Hz, 1H), 7.73 (s, 1H), 7.64 (d, J = |
| | [M + H]⁺. | 1.6 Hz, 1H), 7.51 (s, 1H), 7.46 (t, J = 5.1 Hz, 2H), 7.36- |
| | | 7.32 (m, 1H), 7.29 (s, 1H), 7.12 (d, J = 7.4 Hz, 1H), 4.43- |
| | | 4.33 (m, 2H), 4.21-4.12 (m, 1H), 3.73-3.67 (m, 4H), 3.62- |
| | | 3.57 (m, 1H), 3.14-3.07 (m, 2H), 2.72-2.66 (m, 1H), |
| | | 2.54-2.51 (m, 1H), 2.34-2.28 (m, 2H), 2.23-2.18 (m, |
| | | 1H), 2.10 (s, 3H), 2.06-2.00 (m, 1H). |
| B127 | mass calcd. for | ¹H NMR (400 MHZ, DMSO-d₆) δ 10.88 (s, 1H), 9.34-9.03 |
| | C₄₆H₄₂N₆O₆, 774.3; m/z | (m, 2H), 8.45 (dd, J = 8.3, 2.2 Hz, 1H), 8.08 (d, J = 8.3 Hz, |
| | found, 775.3 [M + H]⁺. | 1H), 7.96 (s, 1H), 7.71 (d, J = 1.6 Hz, 1H), 7.66-7.53 (m, |
| | | 3H), 7.41-7.34 (m, 2H), 7.21 (d, J = 1.3 Hz, 1H), 6.25 (d, |
| | | J = 8.6 Hz, 1H), 4.35 (d, J = 6.0 Hz, 2H), 4.15 (dd, J = 12.2, |
| | | 4.9 Hz, 1H), 4.04-3.91 (m, 2H), 3.72 (s, 3H), 3.66-3.55 |
| | | (m, 2H), 3.52-3.39 (m, 2H), 3.21-3.07 (m, 2H), 3.04- |
| | | 2.89 (m, 1H), 2.77-2.56 (m, 2H), 2.39-2.21 (m, 4H), 2.12- |
| | | 1.99 (s, 3H), 1.84-1.70 (m, 4H). |
| B128 | mass calcd. for | ¹H NMR (400 MHZ, DMSO-d₆) δ 10.88 (s, 1H), 9.40 (d, J = |
| | C₄₁H₃₇N₉O₅, 735.3; m/z | 1.4 Hz, 1H), 9.29 (t, J = 6.0 Hz, 1H), 8.69 (dd, J = 8.2, |
| | found, 736.4 [M + H]⁺. | 2.1 Hz, 1H), 8.17-8.10 (m, 1H), 7.96 (s, 1H), 7.87 (s, 1H), |
| | | 7.71 (d, J = 1.1 Hz, 1H), 7.59-7.55 (m, 2H), 7.36 (dd, J = |
| | | 8.5, 1.5 Hz, 1H), 6.25 (s, 1H), 4.36 (d, J = 5.9 Hz, 2H), 4.15 |
| | | (dd, J = 12.3, 4.9 Hz, 1H), 3.76-3.71 (m, 2H), 3.59 (s, 3H), |
| | | 3.13-3.03 (m, 9H), 2.76-2.67 (m, 1H), 2.58-2.53 (m, |
| | | 1H), 2.37-2.30 (m, 1H), 2.22-2.16 (m, 2H), 2.10-2.06 |
| | | (m, 1H), 2.02-2.01 (m, 2H). |
| B129 | mass calcd. for | ¹H NMR (400 MHz, DMSO-d₆) δ 10.88 (s, 1H), 9.21 (t, J = |
| | C₄₂H₃₄FN₅O₅, 707.3; m/z | 6.0 Hz, 1H), 8.77-8.71 (m, 1H), 8.10-8.04 (m, 2H), |
| | found, 708.3 [M + H]⁺. | 7.96 (s, 1H), 7.73-7.66 (m, 3H), 7.59-7.54 (m, 2H), 7.41- |
| | | 7.34 (m, 2H), 7.25 (d, J = 7.6 Hz, 1H), 5.64 (d, J = 14.0 |
| | | Hz, 1H), 4.35 (d, J = 5.9 Hz, 2H), 4.15 (dd, J = 12.2, 4.8 |
| | | Hz, 1H), 3.71 (s, 3H), 3.66-3.52 (m, 2H), 2.97 (t, J = 6.0 |
| | | Hz, 2H), 2.75-2.67 (m, 1H), 2.59-2.53 (m, 1H), 2.39- |
| | | 2.27 (m, 1H), 2.24-2.16 (m, 1H), 2.13 (s, 3H), 2.12-2.01 |
| | | (m, 2H). |
| B130 | mass calcd. for | ¹H NMR (400 MHZ, DMSO-d₆) δ 10.88 (s, 1H), 9.40 (d, J = |
| | C₄₂H₃₈N₈O₅, 734.3; m/z | 1.8 Hz, 1H), 9.30 (t, J = 6.0 Hz, 1H), 8.70 (dd, J = 8.2, |
| | found, 735.3 [M + H]⁺. | 2.1 Hz, 1H), 8.19-8.12 (m, 1H), 7.96 (s, 1H), 7.85 (s, 1H), |
| | | 7.76 (s, 1H), 7.74-7.69 (m, 1H), 7.60-7.55 (m, 1H), 7.39- |
| | | 7.33 (m, 1H), 7.27 (s, 1H), 4.36 (d, J = 5.9 Hz, 2H), 4.15 |
| | | (dd, J = 12.2, 4.8 Hz, 1H), 3.80-3.73 (m, 2H), 3.67 (s, 3H), |
| | | 3.15-3.05 (m, 3H), 2.76-2.67 (m, 1H), 2.60-2.52 (m, |
| | | 1H), 2.39-2.29 (m, 1H), 2.25-2.16 (m, 2H), 2.13-2.06 |
| | | (m, 4H), 1.26 (d, J = 6.9 Hz, 6H). |
| B131 | mass calcd. for | ¹H NMR (400 MHZ, DMSO-d₆) δ 10.88 (s, 1H), 9.46 (t, J = |
| | C₄₅H₃₈N₈O₇, 802.3; m/z | 6.1 Hz, 1H), 9.42 (d, J = 1.8 Hz, 1H), 8.86 (s, 1H), 8.80 |
| | found, 803.3 [M + H]⁺. | (s, 1H), 8.76 (m, 1H), 8.21 (d, J = 8.2 Hz, 1H), 7.81 (s, 1H), |
| | | 7.63 (s, 1H), 7.47 (d, J = 8.4 Hz, 1H), 7.36-7.32 (m, 1H), |
| | | 7.30 (s, 1H), 7.12 (d, J = 7.5 Hz, 1H), 6.67 (s, 1H), 4.40- |
| | | 4.44 (m, 2H), 4.19-2.21 (m, 1H), 3.71-3.74 (m, 4H), 3.65- |
| | | 3.67 (m, 6H), 3.57-3.62 (m, 2H), 3.31-3.39 (m, 2H), |
| | | 2.76-2.69 (m, 1H), 2.38-2.34 (m, 1H), 2.08-2.00 (m, |
| | | 2H), 1.98-2.05 (m, 3H). |
| B132 | mass calcd. for | ¹H NMR (400 MHZ, DMSO-d₆) δ 10.88 (s, 1H), 9.47 (d, J = |
| | C₄₆H₃₈N₆O₆, 770.85; m/z | 1.7 Hz, 1H), 9.34 (t, J = 5.9 Hz, 1H), 9.01 (s, 1H), 8.83 |
| | found, 771.3 [M + H]⁺. | (s, 1H), 8.78 (dd, J = 8.3, 2.2 Hz, 1H), 8.27-8.20 (m, 2H), |
| | | 8.02-7.95 (m, 2H), 7.74-7.70 (m, 2H), 7.58 (d, J = 8.6 |
| | | Hz, 1H), 7.51 (s, 1H), 7.39-7.35 (m, 2H), 4.38 (d, J = 6.1 |
| | | Hz, 2H), 4.15 (dd, J = 12.1, 4.7 Hz, 1H), 4.01-3.93 (m, |
| | | 2H), 3.75 (s, 3H), 3.49-3.43 (m, 2H), 3.16-3.11 (m, 1H), |
| | | 2.70-2.64 (m, 1H), 2.61-2.56 (m, 1H), 2.34-2.31 (m, |
| | | 1H), 2.11-2.06 (m, 1H), 2.00 (s, 3H), 1.92-1.85 (m, 4H). |
| B133 | mass calcd. for | ¹H NMR (400 MHZ, DMSO-d₆) δ 10.87 (s, 1H), 9.21 (s, |
| | C₄₄H₃₈FN₅O₅, 735.3; m/z | 1H), 8.74 (s, 1H), 8.07 (s, 2H), 7.95 (s, 1H), 7.70 (s, 1H), |
| | found, 736.3 [M + H]⁺. | 7.63-7.55 (m, 2H), 7.41-7.33 (m, 3H), 7.13 (s, 1H), 5.62 |
| | | (d, J = 14.2 Hz, 1H), 4.34 (d, J = 5.1 Hz, 2H), 4.19-4.10 |
| | | (m, 1H), 3.70 (s, 3H), 3.63 (d, J = 8.5 Hz, 1H), 3.54 (s, 1H), |
| | | 2.96 (s, 2H), 2.79-2.76 (m, 2H), 2.69 (d, J = 17.3 Hz, 1H), |
| | | 2.58 (s, 1H), 2.33 (d, J = 8.1 Hz, 1H), 2.18 (s, 1H), 2.10 (s, |
| | | 5H), 1.29-1.26 (m, 3H). |
| B134 | mass calcd. for | ¹H NMR (400 MHz, DMSO-d₆) δ 10.88 (s, 1H), 9.22 (t, J = |
| | C₄₅H₄₀FN₅O₅, 749.3; m/z | 5.9 Hz, 1H), 8.74 (s, 1H), 8.12-8.01 (m, 2H), 7.96 (s, |
| | found, 750.3 [M + H]⁺. | 1H), 7.71 (s, 1H), 7.62-7.54 (m, 2H), 7.42-7.33 (m, 3H), |
| | | 7.18 (s, 1H), 5.61 (d, J = 14.1 Hz, 1H), 4.35 (d, J = 5.8 Hz, |
| | | 2H), 4.19-4.12 (m, 1H), 3.72 (s, 3H), 3.67-3.63 (m, 1H), |
| | | 3.55-3.52 (m, 1H), 3.11-3.05 (m, 1H), 3.00-2.94 (m, |
| | | 2H), 2.75-2.68 (m, 1H), 2.59-2.54 (m, 1H), 2.37-2.28 |
| | | (m, 1H), 2.23-2.16 (m, 2H), 2.11 (s, 3H), 2.05-1.88 (m, |
| | | 1H), 1.32-1.27 (m, 6H). |
| B135 | mass calcd. for | ¹H NMR (400 MHZ, DMSO-d₆) δ 10.90 (s, 1H), 10.88 (s, |
| | C₄₃H₄₀N₈O₆, 764.3; m/z | 1H), 9.45-9.41 (m, 1H), 9.26 (t, J = 5.8 Hz, 1H), 9.13 (t, |
| | found, 765.3 [M + H]⁺. | J = 5.9 Hz, 1H), 8.99-8.95 (m, 1H), 8.74-8.72 (m, 1H), |
| | | 8.72-8.69 (m, 1H), 8.58 (s, 1H), 8.16 (s, 1H), 7.98 (s, 1H), |
| | | 7.96 (s, 1H), 7.74 (s, 1H), 7.69 (s, 1H), 7.61-7.55 (m, 3H), |
| | | 7.45-7.40 (m, 2H), 7.38-7.33 (m, 1H), 7.31 (s, 1H), 7.24 |
| | | (s, 1H), 4.37 (d, J = 5.7 Hz, 2H), 4.28 (d, J = 6.0 Hz, 1H), |
| | | 4.20-4.16 (m, 1H), 4.16-4.14 (m, 1H), 4.01-3.99 (m, |
| | | 1H), 3.98-3.94 (m, 2H), 3.75-3.74 (m, 2H), 3.73-3.72 |
| | | (m, 3H), 3.67-3.67 (m, 1H), 3.65-3.65 (m, 3H), 3.48- |
| | | 3.46 (m, 2H), 3.45-3.43 (m, 1H), 3.02-2.97 (m, 1H), 2.95- |
| | | 2.92 (m, 1H), 2.80-2.76 (m, 1H), 2.75-2.71 (m, 1H), |
| | | 2.70 (s, 3H), 2.61-2.59 (m, 1H), 2.56-2.55 (m, 1H), 2.50- |
| | | 2.49 (m, 2H), 2.39-2.34 (m, 1H), 2.34-2.31 (m, 1H), |
| | | 2.14-2.12 (m, 1H), 2.11-2.09 (m, 1H), 2.08 (s, 3H), 2.07- |
| | | 2.02 (m, 2H), 1.87-1.81 (m, 2H), 1.80-1.76 (m, 4H). |
| B136 | mass calcd. for | ¹H NMR (400 MHZ, DMSO-d₆) δ 11.23 (s, 1H), 9.46 (t, J = |
| | C₄₅H₄₃N₉O₅, 789.3; m/z | 5.8 Hz, 1H), 9.38 (d, J = 1.6 Hz, 1H), 8.67 (dd, J = 8.2, |
| | found, 790.3 [M + H]⁺. | 2.1 Hz, 1H), 8.54-8.46 (m, 1H), 8.15 (d, J = 8.3 Hz, 1H), |
| | | 8.06-7.93 (m, 1H), 7.70-7.69 (m, 1H), 7.45-7.41 (m, |
| | | 2H), 7.40 (s, 1H), 7.28-7.27 (m, 1H), 4.93-4.70 (m, 1H), |
| | | 4.49 (d, J = 5.8 Hz, 2H), 3.99-3.93 (m, 5H), 3.75-3.69 |
| | | (m, 5H), 3.62-3.59 (m, 1H), 3.47-3.46 (m, 1H), 3.15- |
| | | 3.11 (m, 2H), 2.99-2.95 (m, 1H), 2.78-2.73 (m, 1H), 2.68- |
| | | 2.66 (m, 1H), 2.36-2.29 (m, 2H), 2.23-2.17 (m, 2H), |
| | | 2.10 (s, 3H), 1.81-1.76 (m, 4H). |
| B137 | mass calcd. for | ¹H NMR(400 MHZ, DMSO-d₆) δ 10.88 (s, 1H), 9.39 (d, J = |
| | C₄₄H₃₉N₉O₆, 789.30; m/z | 4.0 Hz, 1H), 9.29 (d, J = 5.7 Hz, 1H), 8.69 (s, 1H), 8.19- |
| | found, 790.30 [M + H]⁺. | 8.11 (m, 1H), 7.95 (d, J = 5.4 Hz, 1H), 7.82 (d, J = 5.4 |
| | | Hz, 1H), 7.70 (d, J = 3.9 Hz, 1H), 7.62-7.48 (m, 2H), |
| | | 7.37 (d, J = 3.9 Hz, 1H), 6.10 (d, J = 5.9 Hz, 1H), 4.72- |
| | | 4.58 (m, 4H), 4.35-4.25 (m, 2H), 4.16-4.00 (m, 5H), |
| | | 3.70-3.65 (m, 2H), 3.55 (s, 3H), 3.09-2.98 (m, 2H), |
| | | 2.78-2.65 (m, 1H), 2.57-2.50 (m, 1H), 2.34-2.28 (m, |
| | | 1H), 2.19-2.15 (m, 2H), 2.09-2.05 (m, 1H), 2.01-1.98 |
| | | (m, 3H). |
| B138 | mass calcd. for | ¹H NMR (400 MHZ, DMSO-d₆) δ 10.88 (s, 1H), 9.44 (t, J = |
| | C₄₅H₄₁N₇O₆, 775.3; m/z | 5.9 Hz, 1H), 9.25-9.21 (m, 1H), 8.52 (d, J = 8.2 Hz, 1H), |
| | found, 776.4 [M + H]⁺. | 8.14 (d, J = 8.2 Hz, 1H), 8.08 (s, 1H), 7.61 (s, 1H), 7.46 (d, |
| | | J = 8.3 Hz, 1H), 7.41 (s, 1H), 7.34 (t, J = 7.9 Hz, 1H), 7.28 |
| | | (s, 1H), 7.25 (s, 1H), 7.12 (d, J = 7.4 Hz, 1H), 4.42 (d, J = |
| | | 5.7 Hz, 2H), 4.21 (dd, J = 12.1, 4.8 Hz, 1H), 3.98 (d, J = |
| | | 11.4 Hz, 2H), 3.91-3.86 (m, 1H), 3.73 (s, 3H), 3.71-3.68 |
| | | (m, 1H), 3.49-3.45 (m, 2H), 3.06 (d, J = 7.2 Hz, 2H), 2.99- |
| | | 2.94 (m, 1H), 2.72-2.66 (m, 1H), 2.36-2.31 (m, 2H), |
| | | 2.23-2.13 (m, 2H), 2.07 (s, 3H), 2.04-2.00 (m, 1H), 1.83- |
| | | 1.75 (m, 4H). |
| B139 | mass calcd. for | ¹H NMR (400 MHZ, DMSO-d₆) δ 10.79 (s, 1H), 9.36-9.28 |
| | C₄₅H₄₅N₉O₅Si, 819.3; | (m, 1H), 9.21 (t, J = 6.0 Hz, 1H), 8.61 (dd, J = 8.2, 1.9 Hz, |
| | m/z found, 820.1 | 1H), 8.07 (d, J = 8.2 Hz, 1H), 7.87 (s, 1H), 7.82 (s, 1H), |
| | [M+H]. | 7.64-7.60 (m, 1H), 7.50-7.45 (m, 2H), 7.30-7.25 (m, |
| | | 1H), 6.31 (s, 1H), 4.27 (d, J = 5.8 Hz, 2H), 4.06 (dd, J = |
| | | 12.3, 4.8 Hz, 1H), 3.78-3.72 (m, 4H), 3.65-3.62 (m, 2H), |
| | | 3.50 (s, 3H), 3.01 (t, J = 6.4 Hz, 2H), 2.66-2.59 (m, 1H), |
| | | 2.50-2.45 (m, 1H), 2.28-2.21 (m, 1H), 2.14-2.06 (m, |
| | | 2H), 2.02-1.98 (m, 1H), 1.93 (s, 3H), 0.71-0.63 (m, 4H), |
| | | -0.00 (s, 6H). |
| B140 | mass calcd. for | ¹H NMR (400 MHZ, DMSO-d₆) δ 10.87 (s, 1H), 9.15 (t, J = |
| | C₄₇H₄₃N₅O₆, 773.3; m/z | 5.9 Hz, 1H), 8.90-8.77 (m, 1H), 8.16-8.10 (m, 1H), |
| | found, 774.5 [M + H]⁺. | 8.07-8.00 (m, 1H), 7.95 (s, 1H), 7.70 (s, 1H), 7.61 (s, 1H), |
| | | 7.58-7.53 (m, 2H), 7.39-7.33 (m, 2H), 7.31-7.25 (m, |
| | | 1H), 7.16 (s, 1H), 6.03-5.88 (m, 1H), 4.34 (d, J = 5.9 Hz, |
| | | 2H), 4.17-4.12 (m, 1H), 3.99-3.94 (m, 2H), 3.72 (s, 3H), |
| | | 3.56-3.50 (m, 2H), 3.47-3.42 (m, 3H), 3.06-2.88 (m, |
| | | 4H), 2.72-2.66 (m, 1H), 2.60-2.55 (m, 1H), 2.35-2.30 |
| | | (m, 1H), 2.22-2.16 (m, 1H), 2.09-2.07 (m, 3H), 1.82- |
| | | 1.72 (m, 4H). |
| B141 | mass calcd. for | ¹H NMR (400 MHZ, DMSO-d₆) δ 10.88 (s, 1H), 9.24 (t, J = |
| | C₄₆H₄₃N₇O₆, 789.3; m/z | 6.1 Hz, 1H), 9.18 (s, 1H), 8.40 (s, 1H), 7.71 (s, 1H), 7.47 |
| | found, 790.3 [M + H]⁺. | (d, J = 8.3 Hz, 1H), 7.40 (d, J = 7.2 Hz, 2H), 7.36-7.32 |
| | | (m, 1H), 7.29 (d, J = 4.4 Hz, 2H), 7.12 (d, J = 7.4 Hz, 1H), |
| | | 4.39 (d, J = 5.8 Hz, 2H), 4.22 (dd, J = 12.2, 4.8 Hz, 1H), |
| | | 4.01-3.91 (m, 2H), 3.77-3.68 (m, 4H), 3.62-3.54 (m, |
| | | 1H), 3.49-3.39 (m, 2H), 3.16-3.08 (m, 2H), 3.01-2.91 |
| | | (m, 1H), 2.64 (s, 3H), 2.57-2.52 (m, 1H), 2.42-2.14 (m, |
| | | 4H), 2.10 (s, 3H), 2.07-2.01 (m, 1H), 1.83-1.70 (m, 4H). |
| B142 | mass calcd. for | ¹H NMR (400 MHZ, DMSO-d₆) δ 10.96 (s, 1H), 9.45 (t, J = |
| | C₄₂H₃₈N₁₀O₆, 778.3; m/z | 5.9 Hz, 1H), 9.42-9.38 (m, 1H), 8.70 (dd, J = 8.2, 1.9 |
| | found, 779.0 [M + H]⁺. | Hz, 1H), 8.47 (d, J = 5.9 Hz, 1H), 8.16 (d, J = 8.2 Hz, 1H), |
| | | 7.87 (s, 1H), 7.63 (d, J = 5.7 Hz, 1H), 7.57 (s, 1H), 7.52 (s, |
| | | 1H), 6.51 (s, 1H), 4.49-4.42 (m, 3H), 3.76-3.72 (m, 2H), |
| | | 3.71-3.67 (m, 4H), 3.60 (s, 3H), 3.56-3.51 (m, 4H), 3.10 |
| | | (t, J = 6.2 Hz, 2H), 2.67-2.53 (m, 2H), 2.40-2.32 (m, |
| | | 1H), 2.24-2.15 (m, 3H), 2.03 (s, 3H). |
| B143 | mass calcd. for | ¹H NMR (400 MHZ, DMSO-d₆) δ 10.88 (s, 1H), 9.24-9.19 |
| | C₄₇H₄₂FN₅O₆, 791.3; m/z | (m, 1H), 8.74 (s, 1H), 8.09-8.05 (m, 2H), 7.96 (s, 1H), |
| | found, 792.4 [M + H]⁺. | 7.70 (s, 1H), 7.61-7.56 (m, 2H), 7.41-7.34 (m, 3H), 7.20 |
| | | (s, 1H), 5.62 (d, J = 14.1 Hz, 1H), 4.35 (d, J = 5.9 Hz, 2H), |
| | | 4.17-4.12 (m, 1H), 3.97 (d, J = 11.3 Hz, 2H), 3.72 (s, 3H), |
| | | 3.69-3.63 (m, 1H), 3.56-3.50 (m, 1H), 3.49-3.41 (m, |
| | | 2H), 3.00-2.93 (m, 3H), 2.73-2.67 (m, 1H), 2.61-2.53 |
| | | (m, 1H), 2.37-2.30 (m, 1H), 2.23-2.14 (m, 1H), 2.12- |
| | | 2.09 (m, 4H), 2.03-1.97 (m, 1H), 1.83-1.75 (m, 4H). |
| B144 | mass calcd. for | ¹H NMR (400 MHZ, DMSO-d₆) δ 10.89 (s, 1H), 9.18 (s, |
| | C₄₇H₄₅N₇O₆, 803.3; m/z | 1H), 9.14-9.06 (m, 1H), 8.41 (s, 1H), 7.97 (s, 1H), 7.70 |
| | found, 804.3 [M + H]⁺. | (s, 2H), 7.58 (d, J = 8.4 Hz, 1H), 7.46-7.35 (m, 3H), 7.28 |
| | | (s, 1H), 4.39-4.29 (m, 2H), 4.21-4.14 (m, 1H), 4.00- |
| | | 3.92 (m, 2H), 3.72 (s, 3H), 3.61-3.56 (m, 1H), 3.51-3.39 |
| | | (m, 4H), 3.15-3.09 (m, 2H), 3.07-3.01 (m, 2H), 2.75- |
| | | 2.67 (m, 1H), 2.61-2.56 (m, 1H), 2.36-2.27 (m, 2H), 2.24- |
| | | 2.18 (m, 1H), 2.10 (s, 3H), 2.08-2.03 (m, 1H), 1.83- |
| | | 1.74 (m, 4H), 1.22-1.18 (m, 3H). |
| B145 | mass calcd. for | ¹H NMR (400 MHZ, DMSO-d₆) δ 10.88 (s, 1H), 9.24 (t, J = |
| | C₄₁H₃₂FN₅O₆, 709.2; m/z | 6.0 Hz, 1H), 8.75 (d, J = 1.3 Hz, 1H), 8.08 (s, 2H), 7.95 |
| | found, 710.2 [M + H]⁺. | (s, 1H), 7.81 (s, 1H), 7.70 (dd, J = 9.9, 6.4 Hz, 2H), 7.56 |
| | | (dd, J = 8.5, 4.4 Hz, 2H), 7.38-7.30 (m, 2H), 7.19 (d, J = |
| | | 7.6 Hz, 1H), 5.88 (d, J = 12.8 Hz, 1H), 4.55-4.50 (m, 1H), |
| | | 4.42-4.39 (m, 1H), 4.35-4.30 (m, 2H), 4.15-4.10 (m, |
| | | 1H), 3.82-3.78 (m, 1H), 3.71 (s, 3H), 3.66 (d, J = 12.6 Hz, |
| | | 1H), 2.71-2.68 (m, 1H), 2.58-2.53(m, 1H), 2.37-2.29 |
| | | (m, 1H), 2.15 (s, 3H), 2.11-2.04 (m, 1H). |
| B146 | mass calcd. for | ¹H NMR (400 MHZ, DMSO-d₆) & 10.88 (s, 1H), 9.47 (s, |
| | C₄₅H₃₇N₇O₆, 771.2; m/z | 1H), 9.34 (t, J = 6.0 Hz, 1H), 9.02 (s, 1H), 8.80 (s, 1H), 8.79- |
| | found, 772.2 [M + H]⁺. | 8.75 (m, 1H), 8.25-8.20 (m, 2H), 8.00-7.94 (m, 2H), |
| | | 7.73-7.66 (m, 2H), 7.58 (d, J = 8.6 Hz, 1H), 7.37 (d, J = |
| | | 8.5 Hz, 1H), 7.10 (s, 1H), 6.32 (s, 1H), 4.38 (d, J = 5.8 Hz, |
| | | 3H), 4.25 (d, J = 6.2 Hz, 2H), 4.18-4.13 (m, 1H), 3.90- |
| | | 3.86 (m, 2H), 3.63 (s, 3H), 3.25 (s, 3H), 2.73-2.68 (m, |
| | | 1H), 2.58-2.55 (m, 1H), 2.35-2.30 (m, 1H), 2.11-2.06 |
| | | (m, 1H), 1.89 (s, 3H). |
| B147 | mass calcd. for | ¹H NMR (400 MHZ, DMSO-d₆) δ 10.88 (s, 1H), 9.24 (s, |
| | C₄₂H₃₄FN₅O₇, 739.2; m/z | 1H), 8.76 (s, 1H), 8.03 (d, J = 50.7 Hz, 3H), 7.64 (d, J = |
| | found, 740.2 [M + H]⁺. | 54.6 Hz, 3H), 7.36 (s, 1H), 7.19 (d, J = 7.2 Hz, 1H), 7.03- |
| | | 6.95 (m, 2H), 5.91 (s, 1H), 4.39 (d, J = 35.1 Hz, 4H), 4.16 |
| | | (dd, J = 11.2, 5.9 Hz, 1H), 3.95-3.84 (m, 4H), 3.72-3.62 |
| | | (m, 4H), 2.13-2.05 (m, 4H), 1.33-1.18 (m, 3H). |
| B148 | mass calcd. for | ¹H NMR (400 MHZ, DMSO-d₆) δ 10.91 (s, 1H), 9.41 (t, J = |
| | C₄₅H₄₁N₇O₆, 775.1; m/z | 5.9 Hz, 1H), 9.38-9.33 (m, 1H), 8.66 (dd, J = 8.2, 2.0 |
| | found, 776.2 [M + H]⁺. | Hz, 1H), 8.13 (d, J = 8.2 Hz, 1H), 7.70 (s, 1H), 7.59-7.54 |
| | | (m, 1H), 7.43-7.37 (m, 2H), 7.30-7.23 (m, 4H), 4.43 (d, |
| | | J = 5.9 Hz, 2H), 4.26 (dd, J = 12.5, 5.0 Hz, 1H), 4.01-3.92 |
| | | (m, 2H), 3.74-3.56 (m, 5H), 3.48-3.41 (m, 2H), 3.17- |
| | | 3.08 (m, 2H), 3.01-2.91 (m, 1H), 2.85-2.74 (m, 1H), 2.58- |
| | | 2.53 (m, 1H), 2.40-2.34 (m, 1H), 2.33-2.16 (m, 2H), |
| | | 2.10 (s, 3H), 2.07-2.01 (m, 1H), 1.84-1.74 (m, 4H). |
| B149 | mass calcd. for | ¹H NMR (400 MHz, DMSO-d₆) δ 10.88 (s, 1H), 9.23 (t, J = |
| | C₄₃H₃₆FN₅O₆, 737.3; m/z | 5.9 Hz, 1H), 8.81-8.69 (m, 1H), 8.08 (s, 2H), 7.95 (s, |
| | found, 738.3 [M+H]+ | 1H), 7.74 (s, 1H), 7.72-7.68 (m, 1H), 7.57 (d, J = 8.5 Hz, |
| | | 1H), 7.39 (s, 1H), 7.38-7.34 (m, 1H), 7.21-7.20 (m, 1H), |
| | | 7.18 (d, J = 7.6 Hz, 1H), 5.85 (d, J = 12.9 Hz, 1H), 4.59- |
| | | 4.51 (m, 1H), 4.46-4.40 (m, 1H), 4.38-4.31 (m, 2H), 4.18- |
| | | 4.11 (m, 1H), 3.87-3.80 (m, 1H), 3.71 (s, 3H), 3.67- |
| | | 3.62 (m, 1H), 2.81-2.75 (m, 2H), 2.73-2.65 (m, 1H), 2.59- |
| | | 2.53 (m, 1H), 2.38-2.29 (m, 1H), 2.12 (s, 3H), 2.07- |
| | | 1.99 (m, 1H), 1.29-1.26 (m, 3H). |
| B150 | mass calcd. for | ¹H NMR (400 MHZ, DMSO-d₆) δ 10.88 (s, 1H), 9.36 (d, J = |
| | C₅₁H₅₀N₈O₇, 886.4; m/z | 1.6 Hz, 1H), 9.27 (t, J = 6.0 Hz, 1H), 8.65-8.61 (m, 1H), |
| | found, 887.6 [M+H] . | 8.13 (d, J = 8.3 Hz, 1H), 7.95 (s, 1H), 7.70 (s, 2H), 7.57 (d, |
| | | J = 8.6 Hz, 1H), 7.44-7.33(m, 3H), 7.28 (s, 1H), 4.59- |
| | | 4.56 (m, 1H), 4.35-4.30 (m, 2H), 4.15-4.10 (m, 2H), 3.83- |
| | | 3.80 (m, 2H), 3.72 (s, 4H), 3.59-3.56 (m, 1H), 3.38 (s, |
| | | 2H), 3.12 (s, 3H), 2.95-2.90 (m, 2H), 2.75-2.67 (m, 1H), |
| | | 2.57-2.53 (m, 2H), 2.33-2.29 (m, 2H), 2.21 (s, 1H), 2.10 |
| | | (s, 4H), 1.89 (s, 2H), 1.54 (s, 6H). |
| B151 | mass calcd. for | ¹H NMR (400 MHZ, DMSO-d₆) δ 10.87 (s, 1H), 9.39- |
| | C₄₁H₃₅N₇O₅, 705; m/z | 9.32 (m, 1H), 9.27 (t, J = 6.0 Hz, 1H), 8.65 (dd, J = 8.2, |
| | found, 706 [M + H]⁺. | 2.1 Hz, 1H), 8.13 (d, J = 8.2 Hz, 1H), 7.95 (s, 1H), 7.71- |
| | | 7.68 (m, 2H), 7.57 (d, J = 8.5 Hz, 1H), 7.43-7.40 (m, |
| | | 2H), 7.36 (dd, J = 8.5, 1.6 Hz, 1H), 7.16 (s, 1H), 4.35 (d, J = |
| | | 5.9 Hz, 2H), 4.15 (dd, J = 12.3, 4.8 Hz, 1H), 3.72-3.66 |
| | | (m, 4H), 3.64-3.57 (m, 1H), 3.16-3.09 (m, 2H), 2.73- |
| | | 2.65 (m, 1H), 2.59-2.53 (m, 1H), 2.48 (s, 3H), 2.39- |
| | | 2.27 (m, 2H), 2.26-2.14 (m, 1H), 2.11-2.05 (m, 4H). |
| B152 | mass calcd. for | ¹H NMR (400 MHz, DMSO-d₆) δ 10.78 (s, 1H), 9.10 (s, |
| | C₄₀H₃₉N₇O₅, 697.3; m/z | 1H), 8.50 (s, 1H), 7.83 (s, 1H), 7.55 (s, 1H), 7.51-7.15 (m, |
| | found, 698 [M + H]⁺. | 3H), 7.11-7.04 (m, 2H), 6.76-6.63 (m, 3H), 6.02 (s, 1H), |
| | | 5.39-5.10 (m, 1H), 4.40-4.35 (m, 1H), 4.32-4.28 (m, |
| | | 2H), 3.71 (s, 3H), 3.58 (s, 3H), 2.92-2.74 (m, 2H), 2.68 |
| | | (s, 3H), 2.65-2.60 (m, 3H), 2.14 (s, 3H), 2.07-1.85 (m, |
| | | 1H), 1.36-1.19 (m, 3H). |
| B153 | mass calcd. for | ¹HNMR(400 MHZ, DMSO-d₆) δ 10.88 (s, 1H), 9.24 (t, J = |
| | C₄₁H₃₅N₇O₅, 705.27; m/z | 5.8 Hz, 1H), 9.18 (d, J = 1.8 Hz, 1H), 8.40 (d, J = 1.3 Hz, |
| | found, 706.27 [M + H]⁺. | 1H), 7.76 (s, 1H), 7.70 (t, J = 8.1 Hz, 1H), 7.57 (d, J = 8.6 |
| | | Hz, 1H), 7.47 (d, J = 8.4 Hz, 1H), 7.41 (s, 1H), 7.38-7.28 |
| | | (m, 3H), 7.12 (d, J = 7.4 Hz, 1H), 4.39 (d, J = 5.9 Hz, 2H), |
| | | 4.22 (dd, J = 12.2, 5.0 Hz, 1H), 3.70-3.67 (m, 4H), 3.64- |
| | | 3.57 (m, 1H), 3.14-3.10 (m, 2H), 2.75-2.66 (m, 1H), 2.64 |
| | | (s, 3H), 2.54-2.49 (m, 1H), 2.40-2.29 (m, 2H), 2.21- |
| | | 2.17 (m, 1H), 2.12 (s, 3H), 2.07-2.00 (m, 1H). |
| B154 | mass calcd. for | ¹H NMR (400 MHz, DMSO-d₆) δ 10.88 (s, 1H), 9.39-9.34 |
| | C₄₅H₄₃N₉O₅, 789.3; m/z | (m, 1H), 9.29-9.24 (m, 1H), 8.68-8.63 (m, 1H), 8.15- |
| | found, 790.6 [M + H]⁺. | 8.11 (m, 1H), 7.86 (s, 1H), 7.70 (s, 1H), 7.62-7.58 (m, |
| | | 1H), 7.42-7.38 (m, 3H), 7.29-7.26 (m, 1H), 4.41-4.34 |
| | | (m, 3H), 4.00-3.94 (m, 5H), 3.74-3.68 (m, 4H), 3.62- |
| | | 3.57 (m, 1H), 3.48-3.41 (m, 2H), 3.16-3.08 (m, 2H), 3.00- |
| | | 2.92 (m, 1H), 2.70-2.57 (m, 2H), 2.43-2.30 (m, 2H), |
| | | 2.24-2.12 (m, 2H), 2.11-2.08 (m, 3H), 1.83-1.75 (m, |
| | | 4H). |
| B155 | mass calcd. for | ¹H NMR (400 MHZ, DMSO-d₆) δ 10.89 (s, 1H), 9.25-9.20 |
| | C₄₀H₃₂ClN₇O₅, 725.3; | (m, 2H), 8.53 (d, J = 1.7 Hz, 1H), 7.97 (s, 1H), 7.75 (s, 1H), |
| | m/z found, 726.6 | 7.73-7.68 (m, 2H), 7.60-7.56 (m, 2H), 7.40-7.36 (m, |
| | [M + H]⁺. | 2H), 7.31 (d, J = 7.5 Hz, 1H), 4.34 (d, J = 5.6 Hz, 2H), 4.16 |
| | | (dd, J = 12.2, 4.8 Hz, 1H), 3.73-3.68 (m, 4H), 3.64-3.58 |
| | | (m, 1H), 3.16-3.10 (m, 2H), 2.76-2.68 (m, 1H), 2.60- |
| | | 2.55 (m, 1H), 2.38-2.29 (m, 2H), 2.23-2.17 (m, 1H), 2.12 |
| | | (s, 3H), 2.11-2.05 (m, 1H). |
| B156 | mass calcd. for | ¹H NMR (400 MHZ, DMSO-d₆) δ 11.03 (s, 1H), 9.42 (s, |
| | C₄₃H₄₀N₁₀O₅, 776.3; m/z | 1H), 9.37 (dd, J = 2.1, 0.7 Hz, 1H), 8.68-8.64 (m, 1H), |
| | found, 777.4 [M + H]⁺. | 8.14 (dd, J = 8.3, 0.6 Hz, 1H), 7.80 (dd, J = 9.0, 1.2 Hz, |
| | | 1H), 7.75-7.70 (m, 2H), 7.42 (s, 1H), 7.40 (s, 1H), 7.28 |
| | | (d, J = 1.1 Hz, 1H), 7.26 (dd, J = 7.1, 1.2 Hz, 1H), 4.69 |
| | | (dd, J = 12.3, 4.9 Hz, 1H), 4.40 (d, J = 6.0 Hz, 2H), 3.97 |
| | | (d, J = 11.7 Hz, 2H), 3.75-3.69 (m, 5H), 3.47-3.43 (m, |
| | | 2H), 3.16-3.10 (m, 2H), 2.99-2.92 (m, 1H), 2.84-2.75 |
| | | (m, 1H), 2.69-2.58 (m, 2H), 2.35-2.30 (m, 1H), 2.24- |
| | | 2.18 (m, 1H), 2.11-2.09 (m, 3H), 2.07-2.01 (m, 1H), |
| | | 1.82-1.75 (m, 4H). |
| B157 | mass calcd. for | ¹H NMR (400 MHZ, DMSO-d₆) δ 10.95 (s, 1H), 9.43 (t, J = |
| | C₄₂H₃₈N₈O₅, 734.3; m/z | 5.9 Hz, 1H), 9.37 (d, J = 1.5 Hz, 1H), 8.66 (dd, J = 8.2, |
| | found, 735.6 [M + H]⁺. | 2.0 Hz, 1H), 8.45 (d, J = 5.8 Hz, 1H), 8.14 (d, J = 8.3 Hz, |
| | | 1H), 7.69 (s, 1H), 7.61 (d, J = 5.6 Hz, 1H), 7.51 (s, 1H), |
| | | 7.40 (s, 1H), 7.39 (s, 1H), 7.25 (s, 1H), 4.49-4.45 (m, 1H), |
| | | 4.44 (d, J = 5.7 Hz, 2H), 3.72 (s, 3H), 3.16-3.00 (m, 4H), |
| | | 2.63-2.54 (m, 3H), 2.39-2.33 (m, 2H), 2.19 (d, J = 5.4 |
| | | Hz, 2H), 2.10 (s, 3H), 1.30-1.27 (m, 6H). |
| B158 | mass calcd. for | ¹H NMR (400 MHZ, DMSO-d₆) δ 10.91-10.86 (m, 1H), |
| | C₄₅H₄₀F₂N₈O₅, 810; m/z | 9.47-9.39 (m, 2H), 8.71 (dd, J = 8.2, 2.1 Hz, 1H), 8.17 (d, |
| | found, 811 [M + H]⁺. | J = 8.2 Hz, 1H), 7.82 (s, 1H), 7.75 (s, 1H), 7.46 (d, J = 8.3 |
| | | Hz, 1H), 7.37-7.31 (m, 1H), 7.28 (d, J = 4.9 Hz, 2H), 7.12 |
| | | (d, J = 7.4 Hz, 1H), 4.43 (d, J = 5.8 Hz, 2H), 4.21 (dd, J = |
| | | 12.2, 4.8 Hz, 1H), 3.75 (s, 3H), 3.74-3.70 (m, 2H), 3.14- |
| | | 3.09 (m, 2H), 2.30-2.95 (m, 1H), 2.73-2.66 (m, 1H), 2.58- |
| | | 2.52 (m, 1H), 2.38-2.31 (m, 1H), 2.18-2.09 (m, 6H), |
| | | 2.07-2.00 (m, 4H), 1.99-1.80 (m, 4H). |
| B159 | mass calcd. for | ¹H NMR (400 MHZ, DMSO) 8 10.88 (s, 1H), 9.46-9.38 |
| | C₄₆H₄₂N₈O₆, 802.9; m/z | (m, 2H), 8.72-8.67 (m, 1H), 8.16 (d, J = 8.3 Hz, 1H), |
| | found, 803.5 [M + H]⁺. | 7.76-7.70 (m, 2H), 7.48-7.42 (m, 2H), 7.34 (t, 1H), |
| | | 7.29 (s, 1H), 7.12 (d, J = 7.4 Hz, 1H), 4.43 (d, J = 5.9 Hz, |
| | | 2H), 4.36-4.27 (m, 2H), 4.21 (dd, J = 12.2, 4.9 Hz, 1H), |
| | | 3.81-3.68 (m, 5H), 3.26-3.20 (m, 1H), 3.15-3.09 (m, |
| | | 2H), 2.75-2.66 (m, 1H), 2.56-2.52 (m, 1H), 2.35 (dd, J = |
| | | 12.3, 8.4 Hz, 3H), 2.31-2.17 (m, 4H), 2.12 (s, 3H), |
| | | 2.05-2.00 (m, 1H), 1.70-1.61 (m, 2H), 1.57-1.48 (m, |
| | | 2H). |
| B160 | mass calcd. for | ¹H NMR (400 MHZ, DMSO-d₆) δ 10.88 (s, 1H), 9.84- |
| | C₄₆H₃₉N₇O₇, 801.9; m/z | 9.22 (m, 1H), 9.09-8.68 (m, 1H), 8.62 (s, 1H), 8.20 (d, J = |
| | found, 802.2 [M + H]⁺. | 8.3 Hz, 1H), 7.82 (s, 1H), 7.64 (d, J = 1.5 Hz, 1H), 7.52- |
| | | 7.35 (m, 1H), 7.35-7.16 (m, 1H), 7.12 (d, J = 7.4 Hz, |
| | | 1H), 4.44 (d, J = 5.9 Hz, 1H), 4.21 (dd, J = 12.1, 5.0 Hz, |
| | | 1H), 4.09-3.92 (m, 1H), 3.82 (s, 2H), 3.65 (s, 2H), 3.25- |
| | | 3.00 (m, 1H), 2.77-2.54 (m, 1H), 2.42-2.26 (m, 1H), |
| | | 2.03 (d, J = 1.3 Hz, 2H), 1.92 (dd, J = 10.9, 3.9 Hz, 2H). |
| B161 | mass calcd. for | ¹H NMR (400 MHZ, DMSO-d₆) δ 10.88 (s, 1H), 9.40 (d, J = |
| | C₄₄H₃₉N₇O₅, 745.8; m/z | 6.0 Hz, 1H), 9.37 (d, J = 1.5 Hz, 1H), 8.66 (dd, J = 8.2, |
| | found, 746.2 [M + H]⁺. | 2.1 Hz, 1H), 8.14 (d, J = 8.2 Hz, 1H), 7.69 (s, 1H), 7.46 |
| | | (d, J = 8.3 Hz, 1H), 7.39 (s, 1H), 7.36-7.31 (m, 2H), 7.29 |
| | | (s, 1H), 7.24 (s, 1H), 7.12 (d, J = 7.4 Hz, 1H), 4.42 (d, J = |
| | | 5.9 Hz, 2H), 4.21 (dd, J = 12.2, 4.9 Hz, 1H), 3.72 (s, 3H), |
| | | 3.68 (d, J = 8.9 Hz, 1H), 3.62-3.57 (m, 1H), 3.32 (s, 1H), |
| | | 3.16-3.10 (m, 2H), 2.72-2.66 (m, 1H), 2.54 (s, 1H), |
| | | 2.38-2.30 (m, 4H), 2.25-2.17 (m, 3H), 2.10 (s, 3H), |
| | | 2.05-1.97 (m, 2H), 1.85 (t, J = 9.5 Hz, 1H). |
| B162 | mass calcd. for | ¹H NMR (400 MHz, DMSO-d₆) δ 10.89-10.85 (m, 1H), |
| | C₄₅H₄₃N₉O₆, 805.1; m/z | 9.42 (dd, J = 11.4, 3.8 Hz, 2H), 8.70 (dd, J = 8.2, 2.0 Hz, |
| | found, 805.9 [M + H]⁺. | 1H), 8.16 (d, J = 8.2 Hz, 1H), 7.87 (s, 1H), 7.54 (s, 1H), |
| | | 7.46 (d, J = 8.3 Hz, 1H), 7.36-7.32 (m, 1H), 7.29 (s, 1H), |
| | | 7.12 (d, J = 7.4 Hz, 1H), 6.48 (s, 1H), 4.43 (d, J = 5.8 Hz, |
| | | 2H), 4.37 (s, 1H), 4.21 (dd, J = 12.2, 4.9 Hz, 1H), 3.95- |
| | | 3.88 (m, 2H), 3.75-3.70 (m, 2H), 3.58 (s, 3H), 3.41- |
| | | 3.37 (m, 2H), 3.10 (t, J = 6.5 Hz, 2H), 2.70-2.65 (m, |
| | | 1H), 2.55-2.53 (m, 1H), 2.36-2.32 (m, 1H), 2.21-2.16 |
| | | (m, 2H), 2.02 (s, 4H), 1.53-1.46 (m, 4H), 1.14 (s, 3H). |
| B163 | mass calcd. for | ¹H NMR (400 MHZ, DMSO-d₆) δ 10.88 (s, 1H), 9.46- |
| | C₄₂H₃₆N₈O₆, 748.3; m/z | 9.38 (m, 2H), 8.74-8.68 (m, 1H), 8.17 (d, J = 8.2 Hz, |
| | found, 749.3 [M + H]⁺. | 1H), 7.85 (s, 1H), 7.77 (s, 1H), 7.46 (d, J = 8.4 Hz, 1H), |
| | | 7.34 (t, J = 7.9 Hz, 1H), 7.28 (d, J = 3.0 Hz, 2H), 7.12 (d, |
| | | J = 7.3 Hz, 1H), 4.94-4.87 (m, 4H), 4.52-4.46 (m, 1H), |
| | | 4.43 (d, J = 6.0 Hz, 2H), 4.21 (dd, J = 12.1, 4.7 Hz, 1H), |
| | | 3.81 (s, 3H), 3.76-3.71 (m, 2H), 3.14-3.09 (m, 2H), |
| | | 2.75-2.62 (m, 2H), 2.40-2.28 (m, 2H), 2.24-2.18 (m, |
| | | 1H), 2.13 (s, 3H), 2.06-2.00 (m, 1H). |
| B164 | mass calcd. for | ¹H NMR (400 MHZ, DMSO-d₆) δ 10.88 (s, 1H), 9.48- |
| | C₄₃H₃₈N₈O₅, 746.8; m/z | 9.38 (m, 2H), 8.70 (dd, J = 8.2, 2.0 Hz, 1H), 8.16 (d, J = |
| | found, 747.2 [M + H]⁺. | 8.3 Hz, 1H), 7.78 (s, 1H), 7.73 (s, 1H), 7.49-7.44 (m, |
| | | 1H), 7.38-7.32 (m, 1H), 7.30 (d, J = 6.5 Hz, 1H), 7.19 (s, |
| | | 1H), 7.12 (d, J = 7.4 Hz, 1H), 4.43 (d, J = 5.8 Hz, 2H), |
| | | 4.22 (dd, J = 12.2, 4.9 Hz, 1H), 3.80 (s, 3H), 3.78-3.74 |
| | | (m, 1H), 3.73-3.67 (m, 2H), 3.14-3.08 (m, 2H), 2.74- |
| | | 2.66 (m, 1H), 2.55-2.52 (m, 1H), 2.38-2.23 (m, 6H), |
| | | 2.21-2.16 (m, 1H), 2.11 (s, 3H), 2.05-1.98 (m, 2H), |
| | | 1.93-1.88 (m, 1H). |
| B165 | mass calcd. for | ¹H NMR(400 MHZ, DMSO-d₆) δ 10.89 (s, 1H), 9.35- |
| | C₄₆H₄₃N₇O₅S, 805.9; m/z | 9.04 (m, 1H), 8.40 (d, J = 1.5 Hz, 1H), 7.87 (d, J = 8.1 Hz, |
| | found, 806.2 [M + H]⁺. | 1H), 7.72 (d, J = 14.0 Hz, 1H), 7.42-7.36 (m, 1H), 7.30- |
| | | 7.21 (m, 1H), 4.45-4.33 (m, 1H), 3.97 (d, J = 11.3 Hz, |
| | | 1H), 3.76-3.67 (m, 2H), 3.63-3.54 (m, 1H), 3.50-3.40 |
| | | (m, 2H), 3.12 (s, 1H), 2.94 (d, J = 15.6 Hz, 1H), 2.79- |
| | | 2.67 (m, 1H), 2.64 (s, 2H), 2.55 (d, J = 3.7 Hz, 1H), 2.35 |
| | | (dd, J = 13.0, 4.4 Hz, 1H), 2.22 (s, 1H), 2.08 (d, J = 15.3 |
| | | Hz, 2H), 2.05 (dd, J = 9.2, 4.3 Hz, 1H), 1.77 (d, J = 8.1 |
| | | Hz, 4H) |
| B166 | mass calcd. for | ¹H NMR (400 MHz, DMSO-d₆) δ 10.90-10.85 (m, 1H), |
| | C₄₃H₃₈N₈O₇, 778.8; m/z | 9.47 (t, J = 6.0 Hz, 1H), 9.39 (d, J = 1.8 Hz, 1H), 8.68 (dd, |
| | found, 777.8 [M − H]⁻ . | J = 8.2, 2.1 Hz, 1H), 8.17 (d, J = 8.3 Hz, 1H), 7.91-7.86 |
| | | (m, 2H), 7.46 (d, J = 8.3 Hz, 1H), 7.37-7.31 (m, 1H), |
| | | 7.30-7.27 (m, 2H), 7.12 (d, J = 7.4 Hz, 1H), 4.81-4.58 |
| | | (m, 2H), 4.43 (d, J = 5.9 Hz, 2H), 4.21 (dd, J = 12.2, 4.8 |
| | | Hz, 1H), 4.01-3.86 (m, 4H), 3.76 (s, 3H), 3.50-3.48 (m, |
| | | 1H), 3.05-2.99 (m, 1H), 2.74-2.67 (m, 1H), 2.56-2.52 |
| | | (m, 1H), 2.36-2.30 (m, 1H), 2.12 (s, 3H), 2.05-1.97 (m, |
| | | 2H), 1.89-1.82 (m, 4H). |
| B167 | mass calcd. for | ¹H NMR (400 MHZ, DMSO-d₆) δ 10.88 (s, 1H), 9.45 (t, J = |
| | C₄₆H₃₉N₇O₆, 785.9; m/z | 5.9 Hz, 1H), 9.36 (d, J = 1.6 Hz, 1H), 8.65 (dd, J = 8.2, |
| | found, 786.5 [M + H]⁺. | 2.1 Hz, 1H), 8.15 (d, J = 8.2 Hz, 1H), 8.05 (s, 1H), 7.46 |
| | | (d, J = 8.3 Hz, 1H), 7.38-7.31 (m, 1H), 7.29 (s, 1H), 7.15- |
| | | 7.08 (m, 2H), 6.89 (s, 1H), 4.61 (d, J = 3.9 Hz, 2H), 4.42 |
| | | (d, J = 5.9 Hz, 2H), 4.21 (dd, J = 12.2, 4.9 Hz, 1H), 3.79 |
| | | (s, 2H), 3.59 (s, 3H), 3.31 (s, 3H), 2.74-2.66 (m, 1H), |
| | | 2.60 (s, 3H), 2.56-2.53 (m, 1H), 2.38-2.30 (m, 1H), |
| | | 2.06-2.00 (m, 1H). |
| B168 | mass calcd. for | ¹H NMR (400 MHZ, DMSO-d₆) & 10.88 (s, 1H), 9.48- |
| | C₄₃H₃₉N₉O₆, 777.3; m/z | 9.32 (m, 2H), 8.73-8.64 (m, 1H), 8.16 (d, J = 8.2 Hz, |
| | found, 778.2 [M + H]⁺. | 1H), 7.83 (s, 1H), 7.57 (s, 1H), 7.46 (d, J = 8.3 Hz, 1H), |
| | | 7.35 (t, J = 7.6 Hz, 1H), 7.29 (s, 1H), 7.12 (d, J = 7.4 Hz, |
| | | 1H), 6.10 (s, 1H), 4.43 (d, J = 5.9 Hz, 2H), 4.21 (dd, J = |
| | | 12.1, 4.9 Hz, 1H), 3.90-3.80 (m, 4H), 3.73-3.66 (m, |
| | | 2H), 3.56 (s, 3H), 3.10 (t, J = 6.4 Hz, 2H), 2.73-2.66 (m, |
| | | 1H), 2.54 (d, J = 3.9 Hz, 1H), 2.36-2.30 (m, 1H), 2.22- |
| | | 2.15 (m, 2H), 2.08-2.03 (m, 1H), 2.02 (s, 3H), 1.43 (s, |
| | | 3H). |
| B169 | mass calcd. for | ¹H NMR (400 MHZ, DMSO-d₆) & 10.88 (s, 1H), 9.38 (s, |
| | C₄₅H₄₁N₇O₆, 775.3; m/z | 1H), 9.22 (s, 1H), 8.54-8.47 (m, 1H), 8.16-8.07 (m, |
| | found, 776.4 [M + H]⁺. | 1H), 7.97 (s, 1H), 7.75-7.63 (m, 1H), 7.49-7.44 (m, |
| | | 1H), 7.38-7.28 (m, 1H), 7.22-7.18 (m, 1H), 7.15-7.10 |
| | | (m, 1H), 4.43 (s, 1H), 4.28-4.15 (m, 1H), 4.05-3.89 (m, |
| | | 1H), 3.81-3.65 (m, 1H), 3.02 (s, 1H), 2.79-2.63 (m, |
| | | 1H), 2.58-2.54 (m, 1H), 2.41-2.27 (m, 1H), 2.19-1.98 |
| | | (m, 7H), 1.94-1.79 (m,4H). |
| B170 | mass calcd. for | ¹H NMR (400 MHZ, DMSO-d₆) δ 10.92 (s, 1H), 9.80- |
| | C₄₄H₄₁N₉O₅, 775.3; m/z | 9.04 (m, 2H), 8.66 (dd, J = 8.2, 2.1 Hz, 1H), 8.55 (d, J = |
| | found, 776.2 [M + H]⁺. | 7.0 Hz, 1H), 8.14 (d, J = 8.2 Hz, 1H), 7.70 (s, 1H), 7.41 |
| | | (d, J = 6.2 Hz, 2H), 7.28 (d, J = 1.4 Hz, 1H), 7.12 (d, J = |
| | | 7.0 Hz, 1H), 6.94 (t, J = 7.0 Hz, 1H), 6.81 (s, 1H), 4.37 (d, |
| | | J = 6.0 Hz, 2H), 4.22 (dd, J = 12.3, 4.9 Hz, 1H), 3.97 (d, J = |
| | | 11.5 Hz, 2H), 3.72 (s, 3H), 3.65-3.52 (m, 1H), 3.45 |
| | | (td, J = 11.3, 4.9 Hz, 2H), 3.14 (d, J = 9.4 Hz, 2H), 2.95 (t, |
| | | J = 7.9 Hz, 1H), 2.80-2.62 (m, 1H), 2.55 (d, J = 4.0 Hz, |
| | | 1H), 2.41 (dd, J = 12.7, 4.3 Hz, 1H), 2.37-2.15 (m, 1H), |
| | | 2.10 (d, J = 1.2 Hz, 3H), 2.04-1.96 (m, 2H), 1.78 (dt, J = |
| | | 8.7, 4.6 Hz, 4H). |
| B171 | mass calcd. for | ¹H NMR(400 MHZ, DMSO-d₆) δ 10.87 (s, 1H), 9.40 (t, J = |
| | C₄₃H₃₇N₇O₅, 731.3; m/z | 5.9 Hz, 1H), 9.37 (d, J = 1.4 Hz, 1H), 8.66 (dd, J = 8.2, |
| | found, 732.2 [M + H]⁺ | 2.1 Hz, 1H), 8.14 (d, J = 8.3 Hz, 1H), 7.66 (s, 1H), 7.46 |
| | | (d, J = 8.3 Hz, 1H), 7.39 (s, 1H), 7.36-7.32 (m, 1H), 7.29 |
| | | (s, 1H), 7.25 (s, 1H), 7.12 (d, J = 7.4 Hz, 1H), 7.01 (d, J = |
| | | 1.1 Hz, 1H), 4.42 (d, J = 5.9 Hz, 2H), 4.21 (dd, J = 12.0, |
| | | 4.9 Hz, 1H), 3.73-3.67 (m, 4H), 3.58 (dd, J = 10.5, 6.1 |
| | | Hz, 1H), 3.11 (d, J = 6.0 Hz, 2H), 2.66 (d, J = 6.2 Hz, |
| | | 1H), 2.54 (dd, J = 4.4, 3.1 Hz, 1H), 2.35-2.31 (m, 2H), |
| | | 2.21 (ddd, J = 8.0, 5.6, 1.3 Hz, 1H), 2.11 (dd, J = 6.6, 3.3 |
| | | Hz, 1H), 2.08 (s, 3H), 2.04 (dd, J = 6.6, 3.0 Hz, 1H), 1.06 |
| | | (d, J = 2.5 Hz, 1H), 1.04 (d, J = 2.7 Hz, 1H), 0.95-0.88 |
| | | (m, 1H), 0.83 (dd, J = 7.6, 4.5 Hz, 1H) |
| B172 | mass calcd. for | ¹H NMR (400 MHZ, DMSO-d₆) δ 10.88 (s, 1H), 9.45- |
| | C₄₄H₃₉N₉O₅, 773.3; m/z | 9.38 (m, 2H), 8.73-8.67 (m, 1H), 8.16 (d, J = 8.4 Hz, |
| | found, 774.2 [M + H]⁺. | 1H), 7.87 (s, 1H), 7.54 (s, 1H), 7.46 (d, J = 8.3 Hz, 1H), |
| | | 7.34 (t, J = 7.9 Hz, 1H), 7.29 (s, 1H), 7.12 (d, J = 7.4 Hz, |
| | | 1H), 6.11 (s, 1H), 4.43 (d, J = 5.8 Hz, 2H), 4.21 (dd, J = |
| | | 12.1, 4.8 Hz, 1H), 3.76-3.71 (m, 2H), 3.70-3.66 (m, |
| | | 2H), 3.57 (s, 3H), 3.44-3.38 (m, 2H), 3.10 (t, J = 6.4 Hz, |
| | | 2H), 2.74-2.68 (m, 1H), 2.56-2.53 (m, 1H), 2.38-2.34 |
| | | (m, 1H), 2.21-2.16 (m, 2H), 2.07-2.02 (m, 1H), 2.01 (s, |
| | | 3H), 1.70-1.66 (m, 2H), 0.74 (d, J = 4.5 Hz, 1H), 0.19- |
| | | 0.15 (m, 1H). |
| C1 | mass calcd. for | ¹H NMR (400 MHZ, DMSO-d₆) δ 11.74 (s, 1H), 10.88 (s, |
| | C₄₁H₃₀N₆O₅, 686.2; m/z | 1H), 9.51 (s, 1H), 9.39 (s, 1H), 9.38-9.34 (m, 1H), 8.84- |
| | found, 687.2 [M + H]⁺. | 8.79 (m, 1H), 8.77 (s, 1H), 8.36 (s, 1H), 8.23-8.20 (m, |
| | | 1H), 8.12-8.09 (m, 1H), 8.08-8.04 (m, 1H), 7.96 (s, 1H), |
| | | 7.95-7.90 (m, 2H), 7.72 (s, 1H), 7.70-7.67 (m, 1H), 7.65 |
| | | (s, 1H), 7.59-7.56 (m, 1H), 7.41-7.36 (m, 2H), 4.41- |
| | | 4.35 (m, 2H), 4.18-4.13 (m, 1H), 2.78-2.75 (m, 3H), 2.72 |
| | | (s, 1H), 2.58-2.56 (m, 1H), 2.35-2.31 (m, 1H), 2.12- |
| | | 2.07 (m, 1H). |

### IL. Biological Assessment

The binding potency of the compounds to P300 and CBP were determined using HTRF assay technology. This assay uses full-length p300 expressed in baculovirus expression system with an N-terminal FLAG tag (Active Motif cat #81858). A 4× protein solution was prepared in an assay buffer consisting of 50 mM HEPES pH 7.5, 50 mM NaCl, 0.1% BSA, 1 mM TCEP, and 0.01% Brij-35. A 4× detection solution (MAb Anti FLAG M2-Tb cryptate, Cisbio, Cat #61FG2TLA) was also prepared in the same assay buffer. A fluorescent probe solution consisting of 5-(8-(7-acetyl-3-(tetrahydro-2H-pyran-4-yl)-5,6,7,8-tetrahydroimidazo[1,5-a]pyrazin-1-yl)isoquinolin-3-yl)-N-(2-(2-(2-(3-(3′,6′-dihydroxy-3-oxo-3H-spiro[isobenzofuran-1,9′-xanthen]-5-yl)thioureido)ethoxy)ethoxy)ethyl)picolinamide was made at 2× in the same assay buffer. The final concentrations in the assay were 2 nM of p300, 0.4 nM of Anti-FLAG Tb and 30 nM of the fluorescein probe.

Recombinant CREBBP (CBP), residues 1081-1197, was purchased from Active Motif (Cat #31373). This construct consists of an N-terminal His-tag and a C-terminal FLAG-tag, and is expressed in E. coli. Detection solution in this case consisted of Anti-6His Tb cryptate gold (Cisbio, Cat #61HI2TLB). The final concentrations in the assay were 0.2 nM of CBP (1081-1197), 0.4 nM of Anti-6His Tb and 15 nM of 5-(8-(7-acetyl-3-(tetrahydro-2H-pyran-4-yl)-5,6,7,8-tetrahydroimidazo[1,5-a]pyrazin-1-yl)isoquinolin-3-yl)-N-(2-(2-(2-(3-(3′,6′-dihydroxy-3-oxo-3H-spiro[isobenzofuran-1,9′-xanthen]-5-yl)thioureido)ethoxy)ethoxy)ethyl)picolinamide as a fluorescent probe.

To a 384-well assay plate (Perkin Elmer, Catalog #6008289) was added 5 μL of 4× p300 using a Dragonfly Discovery liquid dispenser (SPT Labtech). The plate was centrifuged at 211×g for 1 min. The plate was sealed and incubated for 15 min at room temperature. Next, 5 μL of 4× Anti-FLAG antibody was added into the assay plate using the Dragonfly Discovery liquid dispenser. Finally, 10 μL of 2× fluorescent probe was added to the assay plate using the same dispenser. The plate was centrifuged at 211×g for 1 min. The plate was resealed and incubated for 60 min at room temperature. Fluorescence values of the solutions were measured at an excitation wavelength of 495 nm and an emission wavelength of 520 nm using an Envision Multilabel Plate Reader (Perkin Elmer).

The CREBBP/CBP assay was performed in similar conditions to the p300 experiments described above.

p300 and CBP degradation potency and Dₘₐₓ were determined using in cell western (ICW) assay technology in H₁₂₉₉ cells or HiBit technology in A549 HiBit cells.

H₁₂₉₉ cell line was plated in Poly-L-Lysine coated and tissue culture treated 384-well flat, clear bottom microplate. Cells were plated at a cell density of 1.0×10⁵ cells per well in assay media comprised of RPMI 1640, 10% FBS, and 1% penicillin/streptomycin. Cells were placed in a 37° C., 5% CO₂, tissue culture incubator and left to equilibrate overnight. The next day cells were treated with serial dilutions of test compound made up of 10 mM stock solutions in DMSO. 384-well plates containing treated cells were then placed back into a 37° C., 5% CO₂, tissue culture incubator and incubated for 16 hrs. After treatment, cell supernatant was removed from each plate and cells were fixed with PBS containing 30% Glyoxal for 1 hour at room temperature. Cell supernatant was then discarded, and cells were permeabilized with PBS containing 0.1% Triton X for 10 minutes at room temperature. Cell supernatant was removed and the cell permeabilization step was repeated two additional times with supernatant removed after each incubation. PBS containing 0.5% SDS was added to each well and incubated for 30 minutes at room temperature. Supernatant was removed and cells were blocked by adding LI-COR Blocking Buffer (LI-COR, catalog number: 927-60001) for 1 hour at room temperature. Cells supernatant was discarded, and LI-COR blocking buffer was added containing 1:1000 dilution of anti-p300 or anti-CBP primary antibody (Cell Signaling: anti-human p300 clone D8Z4E, catalog number 86377S; anti-human CBP clone D6C5, catalog number 7389S). Plates were covered with aluminum foil and incubated overnight at 4° C. Cell supernatant was discarded, and each plate washed 3 times with PBS containing 0.5% Tween with supernatant discarded after each wash. LI—COR blocking buffer was added containing IRDye 800CW goat anti-rabbit IgG secondary antibody (LI-COR; catalog number: 926-32211; 1:1000 dilution) and CellTag 700 stain (LI-COR; catalog number: 926-41090; 1:500 dilution) then incubated for 1 hour at room temperature. Cell supernatant was discarded, and each plate washed 3 times with PBS containing 0.5% Tween with supernatant discarded after each wash. Plates were left to air dry before being scanned with the LI-COR Odyssey CLx instrument.

A549 P300 HiBit and A549 CBP HiBit cells were plated in separate 384-well, TC treated, white-opaque, flat-bottomed microplate. Cells were plated at a cell density of 0.2×10⁴ cells per well in assay media comprised of F-12K, 10% heat inactivated FBS, and 1% penicillin/streptomycin. Assay microplates containing HiBit cells were placed in a 37° C., 5% CO₂, tissue culture incubator and left to equilibrate overnight. The next day cells were treated with serial dilutions of test compound made up of 10 mM stock solutions in DMSO. 384-Well plates containing treated cells were then placed back into a 37° C., 5% CO₂, tissue culture incubator. Assay microplates containing A549 P300 HiBit cells were incubated for 2 hrs post treatment before P300 destruction was assessed, while microplates containing A549 CBP HiBit cells were incubated for 24 hrs post treatment before CBP destruction was determined. On the day of each assay time point, microplates were removed from the tissue culture incubator and prepared HiBit Lytic detection buffer (Promega, cat #N₃O₄₀) was added to each plate. Plates were covered with aluminum foil and incubated for ten minutes at room temperature on an orbital shaker. After incubation, luminescent signal of each plate was measured using the Envision instrument (Perkin Elmer).

Antiproliferative potencies were determined using Cell-Titer Glo (CTG) assay technology in H₁₂₉₉ cells. H₁₂₉₉ wild type, H₁₂₉₉ p300 knock out, and H₁₂₉₉ CBP knock out lines were seeded in 384-well black, tissue culture treated plates at 100 cells per well in assay media comprised of RPMI 1640, 10% FBS, and 1% penicillin/streptomycin. Seeded assay plates were placed in a 37° C., 5% CO₂ incubator and left to equilibrate overnight. The next day, cells were treated with serial dilutions of test compound made up of 10 mM stock solutions in DMSO, with Staurosporine used as a positive control. The 384-well plates containing treated cells were then placed back into a 37° C., 5% CO₂, tissue culture incubator and incubated for 6 days. In addition, an assay plate containing untreated cells was used as the Day 0 control plate and assessed by addition of Cell Titer-Glo Luminescent Cell Viability reagent (Promega, catalog number: G7573) and signal captured using the Perkin Elmer Envision instrument. On day 6, assay plates were removed from the TC incubator and left to equilibrate at room temperature for 10-15 minutes. Cell Titer-Glo Luminescent Cell Viability reagent (Promega, catalog number: G7573) was added to each well and signal captured using the Perkin Elmer Envision instrument.

TABLE E1

| | P300 A549 2 h | P300 A549 2 h |
|---|---|---|
| Compound No. | HiBit DC₅₀ (nM) | HiBit DMax (%) |
| A1 | B | A |
| A2 | C | A |
| A3 | A | A |
| A4 | C | A |
| A5 | B | A |
| A6 | B | B |
| A7 | C | A |
| A8 | C | B |
| A9 | C | B |
| A10 | C | B |
| A11 | B | A |
| A12 | C | D |
| A13 | B | A |
| A14 | B | A |
| A15 | A | A |
| A16 | B | D |
| A17 | E | E |
| A18 | B | A |
| A19 | D | B |
| A20 | B | B |
| A21 | D | B |
| A22 | C | D |
| A23 | B | A |
| A24 | A | A |
| A25 | A | A |
| A26 | C | B |
| A27 | B | B |
| A28 | B | B |
| A29 | B | A |
| A30 | B | A |
| A31 | B | D |
| A32 | B | A |
| A33 | E | E |
| A34 | C | A |
| A37 | E | E |
| A38 | E | E |
| A39 | B | A |
| A40 | B | A |
| A41 | C | A |
| A42 | B | A |
| A43 | D | B |
| A44 | D | B |
| A45 | B | A |
| A46 | C | A |
| A47 | C | A |
| A48 | C | B |
| A49 | C | A |
| A50 | C | A |
| A51 | B | A |
| A52 | D | B |
| A53 | A | A |
| A54 | B | A |
| A55 | B | A |
| A56 | E | E |
| A57 | C | A |
| A58 | B | A |
| A59 | B | A |
| A60 | E | E |
| A61 | B | A |
| A62 | B | A |
| A63 | A | A |
| A64 | B | A |
| A65 | B | A |
| A66 | B | A |
| A67 | B | B |
| A68 | B | A |
| A69 | B | A |
| A70 | B | A |
| A71 | B | A |
| A72 | B | A |
| A73 | B | A |
| A74 | E | E |
| A75 | B | A |
| A76 | E | E |
| A77 | B | B |
| A78 | E | E |
| A79 | B | A |
| A80 | E | E |
| A81 | E | E |
| A82 | B | A |
| A83 | B | A |
| A84 | C | A |
| A85 | B | A |
| A86 | B | A |
| A87 | B | A |
| A88 | E | A |
| A89 | E | A |
| A90 | B | E |
| A91 | C | E |
| A92 | B | A |
| A93 | C | D |
| A94 | B | A |
| A95 | B | A |
| A96 | B | A |
| A97 | B | A |
| A98 | B | A |
| A99 | A | A |
| A100 | B | A |
| A101 | E | E |
| A102 | A | A |
| A103 | B | B |
| A104 | E | E |
| A105 | E | E |
| A106 | B | A |
| A107 | A | A |
| A108 | B | A |
| A110 | B | A |
| A111 | C | A |
| A112 | B | A |
| A113 | C | A |
| A114 | E | E |
| A115 | A | A |
| A116 | B | A |
| A117 | B | A |
| A118 | C | B |
| A119 | B | A |
| A120 | E | E |
| A121 | C | B |
| A122 | A | A |
| A123 | B | B |
| A124 | A | A |
| A125 | C | C |
| A126 | B | D |
| A127 | A | A |
| A128 | B | A |
| A129 | B | A |
| A130 | C | D |
| A132 | B | A |
| A133 | B | A |
| A134 | B | A |
| A135 | B | A |
| A136 | B | A |
| A137 | B | A |
| A138 | A | A |
| A139 | A | A |
| A140 | B | C |
| A141 | B | A |
| A142 | C | A |
| A143 | B | D |
| A144 | E | E |
| A145 | A | A |
| A146 | B | A |
| A147 | E | E |
| A148 | B | A |
| A149 | E | E |
| A150 | B | A |
| A151 | B | A |
| A152 | C | D |
| A153 | B | A |
| A154 | C | B |
| A155 | B | A |
| A156 | B | A |
| A157 | B | A |
| A158 | B | A |
| A159 | C | C |
| A160 | A | A |
| A161 | A | A |
| A162 | A | A |
| A163 | A | A |
| A164 | C | A |
| A165 | B | A |
| A166 | B | D |
| A167 | B | B |
| A168 | B | A |
| A169 | A | A |
| A170 | A | A |
| A171 | B | A |
| A172 | A | A |
| A173 | B | A |
| A174 | B | A |
| A175 | C | A |
| A176 | C | A |
| A177 | C | A |
| A178 | C | D |
| A179 | B | B |
| A180 | B | A |
| A181 | B | A |
| A182 | B | A |
| A183 | A | A |
| A184 | C | A |
| A185 | C | B |
| A186 | E | E |
| A187 | B | A |
| A188 | A | A |
| A189 | B | A |
| A190 | B | B |
| A191 | C | A |
| A192 | B | A |
| A193 | E | E |
| A194 | B | A |
| A195 | B | A |
| A196 | B | A |
| A197 | B | A |
| A198 | E | E |
| A199 | B | A |
| A200 | B | A |
| A201 | B | A |
| A202 | C | B |
| A203 | C | B |
| A204 | B | A |
| A205 | B | A |
| A206 | A | A |
| A207 | A | A |
| A208 | B | A |
| A209 | A | A |
| A210 | A | A |
| A211 | B | A |
| A212 | B | A |
| A213 | A | A |
| A214 | C | B |
| A215 | C | B |
| A216 | B | A |
| A217 | B | A |
| A218 | A | A |
| A219 | A | A |
| A220 | B | A |
| A221 | B | A |
| A222 | B | A |
| A223 | A | A |
| A224 | B | A |
| A225 | E | E |
| A226 | A | A |
| A227 | B | A |
| A228 | B | B |
| A229 | A | A |
| A230 | B | A |
| A231 | B | A |
| A232 | C | C |
| A233 | C | C |
| A234 | B | A |
| A235 | B | A |
| A236 | C | C |
| A237 | A | A |
| A238 | A | A |
| A239 | A | A |
| A240 | C | C |
| A241 | B | A |
| A242 | B | A |
| A243 | B | A |
| A244 | C | C |
| A245 | A | A |
| A246 | B | C |
| A247 | C | D |
| A248 | C | A |
| A249 | C | D |
| A251 | C | B |
| A252 | B | A |
| A253 | A | A |
| A254 | B | A |
| A255 | B | A |
| A256 | B | A |
| A257 | B | B |
| A258 | A | A |
| A259 | B | A |
| A260 | A | A |
| A261 | B | A |
| A262 | C | A |
| A263 | B | A |
| A264 | B | A |
| A265 | C | D |
| A266 | C | B |
| A267 | B | A |
| A268 | C | D |
| A269 | B | A |
| A270 | A | A |
| A271 | A | A |
| B1 | C | B |
| B2 | B | B |
| B3 | C | D |
| B4 | C | B |
| B5 | B | A |
| B6 | C | B |
| B7 | B | D |
| B8 | B | A |
| B9 | B | A |
| B10 | E | E |
| B11 | C | A |
| B12 | E | E |
| B13 | B | A |
| B14 | C | B |
| B15 | C | A |
| B16 | B | A |
| B17 | E | E |
| B18 | C | A |
| B19 | C | A |
| B20 | B | A |
| B21 | B | A |
| B22 | B | A |
| B23 | B | A |
| B24 | B | A |
| B25 | B | A |
| B26 | B | A |
| B27 | B | B |
| B28 | B | A |
| B29 | A | A |
| B30 | C | B |
| B31 | A | A |
| B32 | A | A |
| B33 | B | A |
| B34 | A | A |
| B35 | B | A |
| B36 | B | A |
| B37 | A | A |
| B38 | A | A |
| B39 | B | A |
| B40 | A | A |
| B41 | B | A |
| B42 | B | B |
| B43 | B | A |
| B44 | B | D |
| B45 | B | A |
| B46 | B | B |
| B47 | A | A |
| B48 | A | A |
| B49 | B | A |
| B50 | B | A |
| B51 | B | C |
| B52 | A | A |
| B53 | A | A |
| B54 | A | A |
| B55 | B | A |
| B56 | B | A |
| B57 | E | E |
| B58 | B | A |
| B59 | B | A |
| B60 | B | A |
| B61 | B | B |
| B62 | B | A |
| B63 | B | A |
| B64 | B | A |
| B65 | B | A |
| B66 | A | A |
| B67 | A | A |
| B68 | B | B |
| B69 | B | C |
| B70 | B | B |
| B71 | B | A |
| B72 | B | B |
| B73 | B | A |
| B74 | B | A |
| B76 | B | A |
| B77 | B | A |
| B78 | E | E |
| B79 | B | A |
| B80 | E | E |
| B81 | B | A |
| B82 | B | A |
| B83 | B | A |
| B84 | A | A |
| B85 | E | E |
| B86 | B | A |
| B87 | B | B |
| B88 | B | A |
| B89 | B | A |
| B90 | A | A |
| B91 | C | A |
| B92 | B | A |
| B93 | B | B |
| B94 | A | A |
| B95 | A | A |
| B96 | B | A |
| B97 | D | E |
| B98 | A | A |
| B99 | B | A |
| B100 | B | A |
| B101 | B | A |
| B102 | B | A |
| B103 | B | A |
| B104 | B | A |
| B105 | E | E |
| B106 | A | A |
| B107 | B | B |
| B108 | A | A |
| B109 | A | A |
| B110 | B | A |
| B111 | B | A |
| B112 | A | A |
| B113 | B | A |
| B114 | A | A |
| B115 | B | B |
| B116 | C | C |
| B117 | B | A |
| B118 | B | A |
| B119 | B | B |
| B120 | B | A |
| B121 | B | C |
| B122 | E | E |
| B123 | B | A |
| B124 | A | A |
| B125 | B | A |
| B126 | B | A |
| B127 | B | A |
| B128 | B | A |
| B129 | B | A |
| B130 | B | B |
| B131 | B | A |
| B132 | B | A |
| B133 | B | A |
| B134 | C | A |
| B135 | E | E |
| B136 | B | B |
| B137 | B | B |
| B138 | A | A |
| B139 | D | B |
| B140 | B | B |
| B141 | A | A |
| B142 | A | A |
| B143 | B | A |
| B144 | B | A |
| B145 | B | A |
| B146 | B | A |
| B147 | B | A |
| B148 | B | B |
| B149 | B | A |
| B150 | B | B |
| B151 | B | A |
| B152 | B | A |
| B153 | A | A |
| B154 | B | A |
| B155 | B | A |
| B156 | C | D |
| B157 | B | A |
| B158 | C | A |
| B159 | B | A |
| B160 | B | A |
| B161 | B | A |
| B162 | B | A |
| B163 | A | A |
| B164 | C | A |
| B165 | B | A |
| B166 | B | B |
| B167 | B | A |
| B168 | A | A |
| B169 | A | A |
| B170 | B | D |
| B171 | B | A |
| B172 | C | A |
| C1 | B | A |

Definitions:
DC₅₀: “A”: < 1 nM; “B”: 1-9.9 nM; “C”: 10-99 nM; “D”: 100-999 nM; “E”: >1000 nM
D_{Max}: “A”: > 80%; “B”: 60-79%; “C”: 40-59%; “D”: 20-39%; “E”: < 20%.
“n.d.” not determined

TABLE E2
Selectivity Data for Selected Compounds

| | CBP A549 24 h | CBP A549 24 h |
|---|---|---|
| Compound No. | HiBit DC₅₀ (nM) | HiBit DMax (%) |
| A1 | C | C |
| A2 | E | E |
| A3 | E | E |
| A5 | C | A |
| A10 | E | E |
| A11 | C | D |
| A14 | C | B |
| A15 | E | E |
| A16 | E | E |
| A18 | C | B |
| A23 | E | E |
| A24 | E | E |
| A25 | D | E |
| A29 | E | E |
| A34 | E | E |
| A35 | C | D |
| A36 | E | E |
| A39 | D | D |
| A45 | E | E |
| A51 | E | E |
| A53 | E | E |
| A54 | E | E |
| A55 | E | E |
| A57 | E | E |
| A58 | E | E |
| A61 | E | E |
| A63 | D | E |
| A64 | E | E |
| A66 | E | E |
| A68 | E | E |
| A69 | E | E |
| A70 | E | E |
| A72 | E | E |
| A75 | E | E |
| A79 | E | E |
| A83 | E | E |
| A84 | E | E |
| A86 | E | E |
| A87 | E | E |
| A88 | E | E |
| A89 | E | E |
| A92 | E | E |
| A93 | E | E |
| A95 | E | E |
| A96 | E | E |
| A97 | C | C |
| A99 | E | E |
| A100 | C | D |
| A102 | E | E |
| A108 | E | E |
| A111 | E | E |
| A115 | E | E |
| A119 | E | E |
| A121 | E | E |
| A122 | E | E |
| A124 | E | E |
| A127 | E | E |
| A132 | E | E |
| A133 | D | E |
| A136 | E | E |
| A138 | E | E |
| A139 | E | E |
| A145 | E | E |
| A150 | E | E |
| A155 | E | E |
| A158 | C | C |
| A160 | E | E |
| A161 | D | E |
| A162 | D | E |
| A163 | E | E |
| A164 | E | E |
| A165 | E | E |
| A169 | E | E |
| A170 | E | E |
| A171 | E | E |
| A172 | D | E |
| A173 | E | E |
| A174 | E | E |
| A175 | E | E |
| A177 | E | E |
| A178 | E | E |
| A180 | E | E |
| A181 | D | E |
| A182 | E | E |
| A185 | E | E |
| A189 | E | E |
| A191 | E | E |
| A194 | E | E |
| A195 | E | E |
| A197 | E | E |
| A199 | D | E |
| A202 | E | E |
| A203 | E | E |
| A204 | B | B |
| A207 | D | E |
| A208 | C | B |
| A210 | E | E |
| A211 | E | E |
| A212 | E | E |
| A213 | E | E |
| A216 | E | E |
| A219 | D | E |
| A221 | E | E |
| A223 | D | E |
| A226 | D | E |
| A234 | E | E |
| A237 | D | E |
| A238 | B | C |
| A243 | E | E |
| A247 | E | E |
| A248 | E | E |
| A251 | E | E |
| A254 | E | E |
| A255 | E | E |
| A258 | E | E |
| A259 | E | E |
| A260 | E | E |
| A261 | E | E |
| A263 | E | E |
| A269 | C | A |
| A270 | B | B |
| A271 | B | B |
| B2 | E | E |
| B4 | D | C |
| B5 | E | E |
| B7 | E | E |
| B8 | C | C |
| B9 | C | C |
| B16 | D | D |
| B20 | E | E |
| B23 | D | E |
| B24 | E | E |
| B25 | C | D |
| B26 | E | E |
| B28 | D | E |
| B29 | D | E |
| B30 | E | E |
| B32 | B | B |
| B35 | C | D |
| B37 | E | E |
| B38 | E | E |
| B39 | E | E |
| B42 | E | E |
| B47 | E | E |
| B48 | D | E |
| B49 | E | E |
| B51 | E | E |
| B53 | E | E |
| B54 | E | E |
| B55 | E | E |
| B56 | E | E |
| B58 | E | E |
| B59 | E | E |
| B60 | E | E |
| B61 | E | E |
| B62 | E | E |
| B63 | C | D |
| B64 | E | E |
| B66 | E | E |
| B67 | D | E |
| B68 | E | E |
| B69 | E | E |
| B71 | E | E |
| B72 | E | E |
| B73 | E | E |
| B74 | E | E |
| B76 | D | E |
| B77 | E | E |
| B79 | E | E |
| B81 | C | C |
| B82 | E | E |
| B83 | D | C |
| B84 | D | E |
| B87 | E | E |
| B88 | E | E |
| B90 | E | E |
| B92 | C | D |
| B94 | B | D |
| B96 | D | E |
| B98 | E | E |
| B102 | E | E |
| B103 | D | E |
| B106 | D | E |
| B107 | E | E |
| B108 | D | E |
| B110 | E | E |
| B111 | C | C |
| B112 | D | E |
| B115 | E | E |
| B117 | D | E |
| B119 | E | E |
| B123 | E | E |
| B124 | B | C |
| B127 | E | E |
| B131 | E | E |
| B132 | E | E |
| B136 | E | E |
| B137 | E | E |
| B138 | D | E |
| B141 | C | D |
| B142 | C | D |
| B148 | E | E |
| B153 | D | E |
| B154 | B | B |
| B156 | E | E |
| B157 | C | D |
| B159 | D | D |
| B160 | E | E |
| B161 | E | E |
| B162 | E | E |
| B163 | E | E |
| B164 | E | E |
| B165 | C | D |
| B166 | E | E |
| B167 | B | B |
| B168 | E | E |
| B169 | B | C |
| B166 | E | E |
| B167 | B | B |
| B168 | E | E |
| B169 | B | C |
| C1 | E | E |

Definitions:
DC₅₀: “A”: < 1 nM; “B”: 1-9.9 nM; “C”: 10-99 nM; “D”: 100-999 nM; “E”: >1000 nM
D_{Max}: “A”: > 80%; “B”: 60-79%; “C”: 40-59%; “D”: 20-39%; “E”: < 20%.
“n.d.” not determined

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of Formula I:
T-L-C (I),
or a pharmaceutically acceptable salt, solvate, or stereoisomer thereof, wherein:
C is of Formula I-1 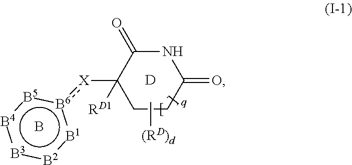
wherein: [symbol]
denotes a single bond or a double bond;
B¹ is CR^{B1}, N, NR^{B1′}, O, or S;
B² is CR^{B2}, N, NR^{B2′}, O, or S;
B³ is absent, CR^{B3}, or N;
B⁴ is CR^{B4}, N, NR^{B4′}, O, or S;
B⁵ is CR^{B5}, N, NR^{B5′}, O, or S;
B⁶ is C or N;
R^{B1}, R^{B2}, R^{B3}, R^{B4}, and R^{B5} are independently 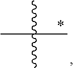
hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}, and one of R^{B1}, R^{B2}, R^{B3}, R^{B4}, and R^{B5} is 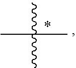
wherein 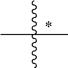
denotes attachment to L; or
R^{B4} and R^{B5}, together with the carbon atoms to which they are bonded, form 3- to 12-membered heterocyclyl or 5- or 6-membered heteroaryl, wherein the heterocyclyl or heteroaryl is optionally substituted with one or more R^{u}, or optionally attached to L;
R^{B1′}, R^{B2′}, R^{B4′}, and R^{B5′} are independently hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
X is absent, CR^{X1}, C(R^{X1})₂, NR^{X2} or O, as valency permits;
each R^{X1} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
two R^{X1} together form an oxo; or
two R^{X1}, together with the carbon atom to which they are bonded, form C₃₋₄ carbocyclyl or 3- to 4-membered heterocyclyl, wherein the carbocyclyl or heterocyclyl is optionally substituted with one or more R^{u};
R^{X2} is hydrogen, C₁₋₃ alkyl, C₃₋₄ carbocyclyl, or 3- to 4-membered heterocyclyl, wherein the alkyl, carbocyclyl, or heterocyclyl is optionally substituted with one or more R^{u}; or
R^{X1} and R^{B5}, R^{X1} and R^{B5′}, R^{X2} and R^{B5}, or R^{X2} and R^{B5′}, together with the atoms to which they are bonded, form Ring C, wherein Ring C is optionally substituted C₆ aryl, optionally substituted 5- to 6-membered heteroaryl, optionally substituted 5- to 8-membered heterocycle, or optionally substituted C₅₋₈ carbocycle;
R^{D1} is hydrogen, deuterium, halogen, C₁₋₆ alkyl, or C₁₋₆ haloalkyl; or
R^{D1} and one R^{D}, together with the carbon atoms to which they are bonded, form C₃₋₄ carbocycle or 3- to 4-membered heterocycle, wherein the carbocycle or heterocycle is optionally substituted with one or more R^{u};
each R^{D} is independently oxo, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; or
two R^{D}, together with the carbon atom(s) to which they are bonded, form C₃₋₅ carbocyclyl or 3- or 5-membered heterocyclyl, wherein the carbocyclyl or heterocyclyl is optionally substituted with one or more R^{u};
d is an integer from 0 to 6, as valency permits; and
q is an integer from 0 to 2,
wherein when X is NR^{X2} or O, and L″ is absent, then i) R^{X2} and R^{B5}, together with the atoms to which they are bonded, form Ring C; ii) B³ is absent; or iii) R^{D1} and one R^{D}, together with the carbon atoms to which they are bonded, form C₃₋₄ carbocycle or 3- to 4-membered heterocycle, wherein the carbocycle or heterocycle is optionally substituted with one or more R^{u};
T is
i) of Formula I-3-i 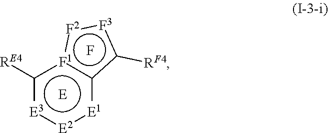
wherein:
E¹ is 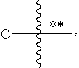
CR^{E1}, or N;
E² is 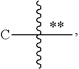
CR^{E2}, or N;
E³ is 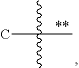
CR^{E3}, or N; 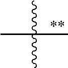
denotes attachment to L;
R^{E1}, R^{E2}, R^{E3}, and R^{E4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}, —N^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}—C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
F¹ is C or N;
F² is NR^{F2′} or CR^{F2};
R^{F2′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}
R^{F2} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
F³ is CR^{F3}, N, or NR^{F3′};
R^{F3} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R^{F3′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R^{F4} is —C(═O)NR^{3a}R^{3b} or 5- to 6-membered heteroaryl optionally substituted with one or more R^{u}; and
R^{3a} and R^{3b} are independently hydrogen, C₁₋₆ alkyl, C₂₋₆ alkenyl, C₂₋₆ alkynyl, C₃₋₁₂ carbocyclyl, 3to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R; or
R^{3a} and R^{3b}, together with the nitrogen atom to which they are attached, form 3- to 12-membered heterocycle or 5- to 10-membered heteroaryl, wherein the heterocycle or heteroaryl is optionally substituted with one or more R^{u};
ii) of Formula I-3-ii′ 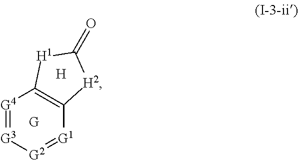
wherein:
G1 is 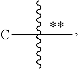
N, or CR^{G1};
G2 is 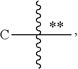
N, or CR^{G2};
G3 is 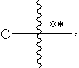
or CR^{G3};
G4 is 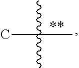
N, or CR^{G4}; 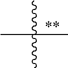
denotes attachment to L;
R^{G1}, R^{G2}, R^{G3}, and R^{G4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—N^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}—C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
H¹ is C(R^{H1})₂, NRH^{1′}, or O;
H² is C(R^{H2})₂, NRH^{2′}, or O;
each occurrence of R^{H11} and R^{H2} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl wherein the alkyl, alkoxy, alkylamino, alkenyl, alkynyl, carbocyclyl, heterocyclyl, aryl, or heteroaryl is optionally substituted with one or more R^{u}; or
two R^{H1} or two R^{H2}, together with the carbon atom to which they are attached, form C₃₋₆ carbocycle or 3- to 6-membered heterocycle, wherein the carbocycle or heterocycle is optionally substituted with one or more R^{u}; and
R^{H1′} and R^{H2′} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
vi) of Formula I-3-vi 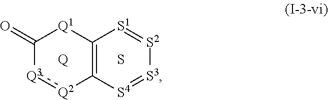
wherein:
Q¹ is NR^{Q1′}, C(R^{Q1})₂, or O;
R^{Q1′} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
each R^{Q1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{Q1}, together with the carbon atom to which they are attached, form C₃₋₆ carbocycle or 3- to 6-membered heterocycle, wherein the carbocycle or heterocycle is optionally substituted with one or more R^{u}
when [symbol] is a double bond, then Q² is N or CR^{Q2}; and Q³ is N or CR^{Q3};
when [symbol] is a single bond, then Q² is NR^{Q2′}, C(R^{Q2})₂, or O; Q³ is NR^{Q3′}, C(R³)₂, or O;
R^{Q2′} and R^{Q3′} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
each occurrence of R^{Q2} and R^{Q3} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two R^{Q2} or two RQ, together with the carbon atom to which they are attached, form C₃₋₆ carbocycle or 3- to 6-membered heterocycle, wherein the carbocycle or heterocycle is optionally substituted with one or more R^{u};
S¹ is CR^{S1} or N;
S² is 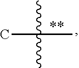
CR^{S2}, or N;
S³ is 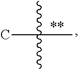
CR^{S3}, or N;
S⁴ is 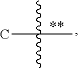
CR^{S4}, or N; 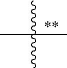
denotes attachment to L; and
R^{S1}, R^{S2}, R^{S3}, and R^{S4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}—NR^{b}C(═O)NR^{c}R^{d}—N^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
L is of Formula I-2 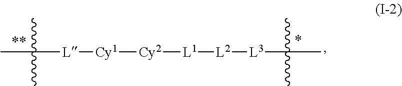
wherein: 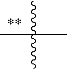
denotes attachment to T and 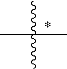
denotes attachment to C;
L″ is absent; or
L″ is C₁₋₃ alkylene, —C(═O)—, —C(═O)N(R^{L})—, —C(═O)O—, —N(R^{L})—, —O—, —S—, or —S(═O)₂—, wherein the alkylene is optionally substituted with one or more R^{u};
R^{L} is hydrogen, C₁₋₆ alkyl, C₁₋₆ haloalkyl, C₃₋₆ carbocyclyl, or 3- to 6-membered heterocyclyl, wherein the alkyl, haloalkyl, carbocyclyl, or heterocyclyl is optionally substituted with one or more R^{u};
wherein:
i) when L″ is C₁₋₃ alkylene, —C(═O)—, —C(═O)N(R^{L})—, —C(═O)O—, —N(R^{L})—, —O—, —S—, or —S(═O)₂, wherein the alkylene is optionally substituted with one or more R^{u}, then Cy¹ is optionally substituted C₆ arylene or 5- to 6-membered heteroarylene;
ii) when L″ is absent, then
Cy¹ is 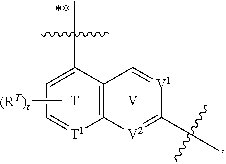
wherein: 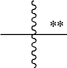
denotes attachment to T;
T¹ is CR^{T1} or N;
V¹ is CR^{V1} or N;
V² is CR^{V2} or N;
R^{T1}, R^{V1}, and R^{V2} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
each R^{T} is independently halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
t is an integer selected from 0 to 6, as valency permits,
wherein R^{T} may be present on either Ring T or Ring V;
or
Cy¹ is 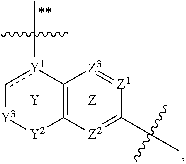
wherein:
when [symbol] is single bond, Y¹ is N or CR^{Y1}; when [symbol] is double bond, Y¹ is C; 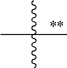
denotes attachment to T;
R^{Y1} is hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
Y² is C(R^{Y2})₂, O, or N(R^{Y2′} each R^{Y2} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; or
two R^{Y2} together form an oxo;
R^{Y2′} is hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
Y³ is C(R^{Y3})₂ or N(R);
each R^{Y3} is independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
R^{Y3′} is hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
Z¹ is CR^{Z1} or N;
Z² is CR^{Z2} or N;
Z³ is CR^{Z3} or N;
R^{Z1}, R^{Z2}, R^{Z3} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u};
Cy² is 5- to 6-membered heteroarylene optionally substituted with one or more R^{u};
L¹ is —C(═O)NH— or 5- to 6-membered heteroaryl optionally substituted with one or more R^{u};
L² is —C(R^{L2})₂—, wherein
each R^{L2} is independently hydrogen, -deuterium, C₁₋₃ alkyl, or C₁₋₃ haloalkyl; or
two geminal R^{L2}, together with the carbon atom to which they are bonded, form C₃₋₅ carbocyclyl or 3- to 5-membered heterocyclyl, wherein the carbocyclyl or heterocyclyl is optionally substituted with one or more R^{u};
and
L³ is E-ethenylene, ethynylene, C₆ arylene, or 5- to 6-membered heteroarylene, wherein the E-ethenylene, arylene, or heteroarylene is optionally substituted with one or more R^{u};
wherein:
each R^{u}; is independently oxo, deuterium, halogen, —CN, —NO₂, —OH, —NH₂, C₁₋₆ alkyl, C₁₋₆ alkoxy, C₁₋₆ alkylamino, C₃₋₄ carbocyclyl, 3- to 4-membered heterocyclyl, —C(═O)R^{a}, or —S(═O)₂R^{a};
each R^{a} is independently C₁₋₆ alkyl, C₃₋₆ carbocyclyl, or 3- to 6-membered heterocyclyl;
each R^{b} is independently hydrogen, C₁₋₆ alkyl, C₃₋₆ carbocyclyl, or 3- to 6-membered heterocyclyl; and
each R^{c} and R^{d} is independently hydrogen, C₁₋₆ alkyl, C₃₋₆ carbocyclyl, or 3- to 6-membered heterocyclyl; or
R^{c} and R^{d}, together with the nitrogen atom to which they are attached, form 3- to 6-membered heterocyclyl,
wherein each occurrence of R^{a}, R^{b}, R^{c}, and R^{d} is independently and optionally substituted with one or more R^{Z}; and
each R^{Z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl.

2. (canceled)

3. The compound of claim 1, wherein C is of Formula I-1-i, I-1-ii, or I-1-iii 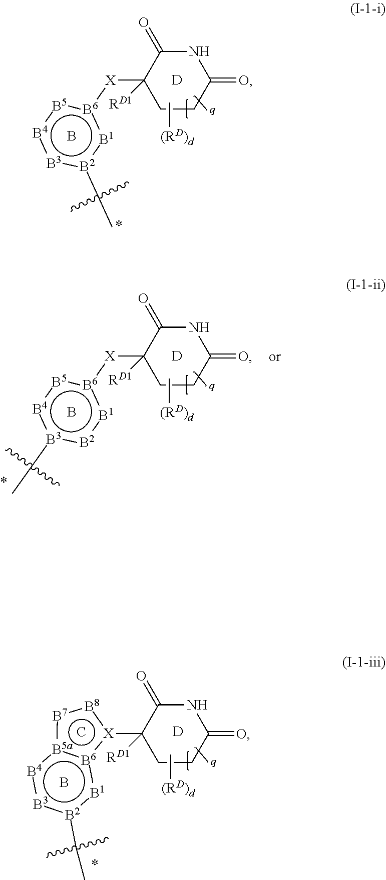
wherein when C is of Formula I-1-i or I-1-ii:
R^{X1} and R^{B5}, R^{X1} and R^{B5′}, R^{X2} and R^{B5}, or R^{X2} and R^{B5′}, together with the atoms to which they are bonded, do not form Ring C, and R^{B2} or R^{B3} is 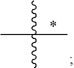
and
when C is of Formula I-1-iii:
B^{5a} is C or N:
B⁷ is CR^{B7}, N, NR^{B7′} O, or S;
B⁸ is CR^{B8}, N, NR^{B8′}, O, or S;
R^{B7} and R^{B8} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}; and
R^{B7′} and R^{B8′} are independently hydrogen, C₁₋₆ alkyl, C₃₋₄ carbocyclyl, 3- to 4-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u},
wherein at least one of B^{5a}, B⁶, B⁷, B⁸, and X is N, NR^{B7′} NR^{B8′}, O, or S.

4.-5. (canceled)

6. The compound of claim 1, wherein C is of Formula I-1-i″ 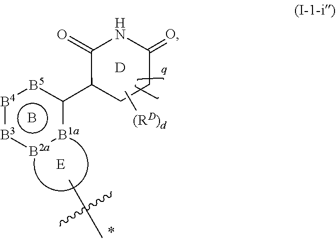
wherein:
Ring E is optionally substituted 5- or 6-membered heteroaryl or optionally substituted 5- or 6-membered heterocycle, and
B^{1a} and B^{2a} are independently C or N.

7. The compound of claim 1, wherein C is of Formula I-1-i″-1

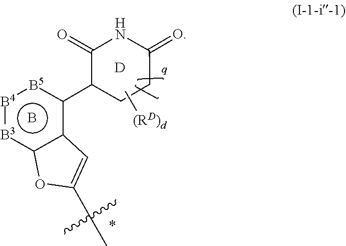

8.-9. (canceled)

10. The compound of claim 1, wherein C is of Formula I-1-iii-1, I-1-iii-2, I-1-iii-3, I-1-iii-4, or I-1-iv-1 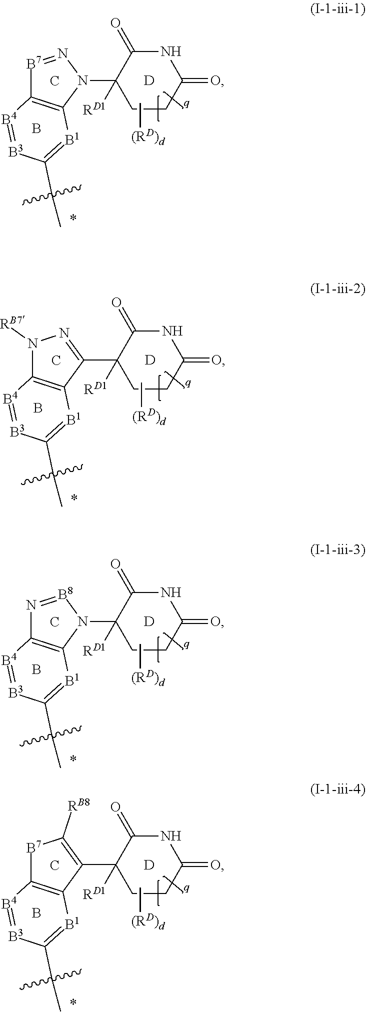 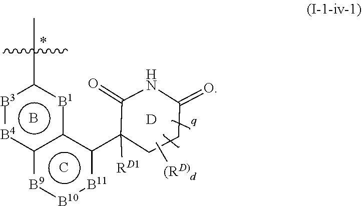
wherein:
when C is of Formula I-1-iii-4, B⁷ is O or S; and
when C is of Formula I-1-iv-1,
B⁹ is CR^{B9} or N;
B¹⁰ is CR^{B10} or N;
B¹¹ is CR^{B11} or N; and
R^{B9}, R^{B10}, and R^{B11} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl, wherein the alkyl, alkoxy, alkylamino, carbocyclyl, or heterocyclyl is optionally substituted with one or more R^{u}.

11.-16. (canceled)

17. The compound of any one of claim 1, wherein R^{D1} is hydrogen, d is 0, and q is 1.

18.-37. (canceled)

38. The compound of claim 1, wherein T is of Formula I-3-ii-a

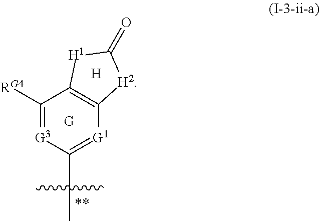

39. The compound of claim 38, wherein:
G1 is CR^{G1}, and G3 is CR^{G3}, wherein R^{G1} and R^{G3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
G4 is CR^{G4}, and R^{G4} is hydrogen, halogen, C₁₋₆ alkyl, C₃₋₁₂ carbocyclyl, 3- to 12-membered heterocyclyl, or 5- to 6-membered heteroaryl, wherein the alkyl, carbocyclyl, or heterocyclyl, or heteroaryl is optionally substituted with one or more R^{u}; and
H¹ is NR^{H1′}, and H² is NR^{H2′}, wherein R^{H11′} and R^{H2′} are independently C₁₋₆ alkyl.

40.-44. (canceled)

45. The compound of claim 1, wherein T is of Formula I-3-vi-a or I-3-vi-b

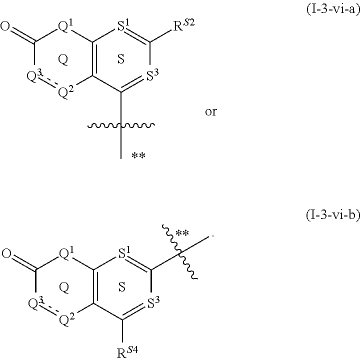

46. (canceled)

47. The compound of claim 45, wherein:
R^{S2} is hydrogen, C₁₋₆ alkyl, C₁₋₆ alkoxy, or 3-12-membered heterocyclyl optionally substituted with one or more R^{u};
R^{S4} is hydrogen or C₁₋₆ alkyl;
Q¹ is NR^{Q1′}, wherein R^{Q1′} is hydrogen or C₁₋₆ alkyl; and
when [symbol] is a double bond, Q² is N or CR^{Q2} and Q³ is CR^{Q3}, wherein R^{Q2} and R^{Q3} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl;
when [symbol] is a single bond, Q² is C(R^{Q2})₂ and Q³ is NR^{Q3′} or C(R^{Q3})₂, wherein each R^{Q2} and R^{Q3} are independently hydrogen, halogen, —CN, —NO₂, —OH, —NH₂, C₁₋₆ alkyl, C₁₋₆ alkoxy, C₁₋₆ alkylamino, C₃₋₄ carbocyclyl, or 3- to 4-membered heterocyclyl; and R^{Q3}'S hydrogen, C₁₋₆ alkyl, C₃₋₄ carbocyclyl, or 3- to 4-membered heterocyclyl;
S¹ is CR^{S1}, and S³ is CR^{S3}, wherein R^{S1} and R^{S3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroarvl, wherein the alkyl, alkoxy, alkylamino, alkenyl, alkynyl, carbocyclyl, heterocyclyl, arvl, or heteroaryl is optionally substituted with one or more R^{u}.

48.-56. (canceled)

57. The compound of claim 1, wherein L″ is absent: L² is CH₂; and L³ is 1,4-phenylene, ethynylene or E-ethylene.

58.-60. (canceled)

61. The compound of claim 1, wherein Cy¹ is

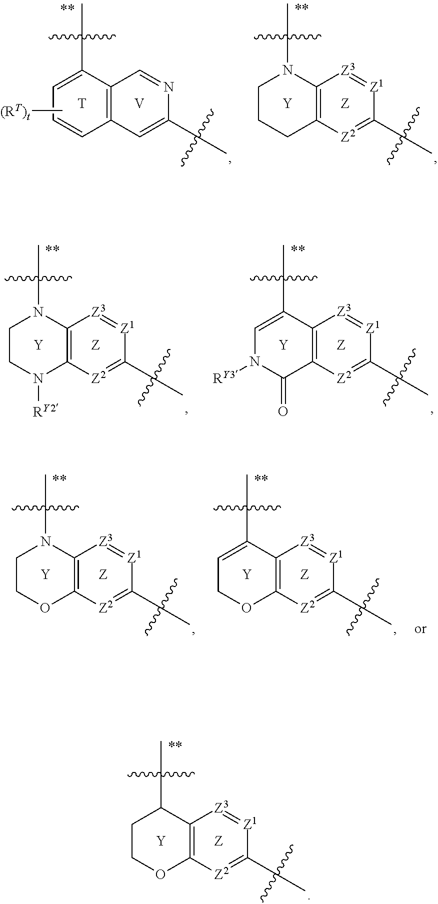

62.-74. (canceled)

75. The compound of claim 1, wherein Cy² is 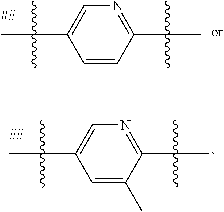
wherein 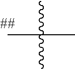
denotes attachment to Cy¹.

76.-77. (canceled)

78. A compound selected from the compounds in Tables 1-4 or a pharmaceutically acceptable salt thereof.

79. (canceled)

80. A pharmaceutical composition comprising the compound of claim 1, and a pharmaceutically acceptable excipient.

81. A method of degrading a protein in a subject or biological sample comprising administering the compound of claim 1 to the subject or contacting the biological sample with the compound of claim 1.

82.-84. (canceled)

85. A method of treating a p300-mediated disorder comprising administering to a patient in need thereof a compound of claim 1.

86.-87. (canceled)

88. The method of claim 85, wherein the p300-mediated disorder is cancer, an inflammatory disorder, or an autoimmune disease.

89. The compound of claim 1, wherein the compound of Formula (I-A) 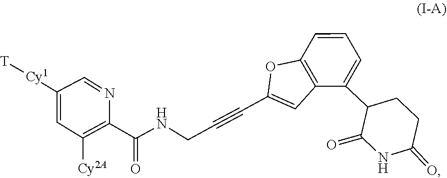
wherein:
Cy^{2A} is hydrogen or C₁₋₆ alkyl; and
T is of Formula I-3-ii′ or I-3-vi.

90. The compound of claim 78, wherein the compound is selected from: Compound Nos. A169, A170, A173, A174, A181, A189, A259, B28, B37, B38, B47, B48, B74, and B163 and pharmaceutically acceptable salts thereof.
